(19) United States
(12) Patent Application Publication
(10) Pub. No.: US 2023/0173078 A1
(43) Pub. Date: Jun. 8, 2023

(54) SMARCA DEGRADERS AND USES THEREOF

(71) Applicant: Kymera Therapeutics, Inc., Watertown, MA (US)

(72) Inventors: Yi ZHANG, Belmont, MA (US); Paul R. FLEMING, Lexington, MA (US); Xiao ZHU, Winchester, MA (US); Matthew M. WEISS, Boston, MA (US)

(21) Appl. No.: 17/851,528

(22) Filed: Jun. 28, 2022

Related U.S. Application Data
(60) Provisional application No. 63/313,685, filed on Feb. 24, 2022.
(60) Provisional application No. 63/215,789, filed on Jun. 28, 2021.

(51) Int. Cl. A61K 47/55; A61K 47/54; A61P 35/00; A61P 11/00

(52) U.S. Cl. CPC A61K 47/55; A61K 47/545; A61P 35/00; A61P 11/00

(57) ABSTRACT

The present invention provides compounds, compositions thereof, and methods of using the same.

### CROSS-REFERENCE TO RELATED APPLICATIONS

This application claims the benefit of priority to U.S. Provisional Appl. Nos. 63/215,789, filed Jun. 28, 2021, and 63/313,685, filed Feb. 24, 2022, the entirety of each of which is herein incorporated by reference.

### TECHNICAL FIELD OF THE INVENTION

The present invention relates to compounds and methods useful for the modulation of one or more SWI/SNF-related matrix-associated actin-dependent regulator of chromatin subfamily A (“SMARCA”) and/or polybromo-1 (“PB1”) protein via ubiquitination and/or degradation by compounds according to the description provided herein. The disclosure also provides pharmaceutically acceptable compositions comprising compounds of the present description and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See e.g., Li et al. “Genome-wide and functional annotation of human E3 ubiquitin ligases identifies MULAN, a mitochondrial E3 that regulates the organelle's dynamics and signaling.” PLOS One 2008, (3)1487; Berndsen et al. “New insights into ubiquitin E3 ligase mechanism” Nat. Struct. Mol. Biol. 2014, 21:301; Deshaies et al. “RING domain E3 ubiquitin ligases” Ann. Rev. Biochem. 2009, 78:399; Spratt et al. “RBR E3 ubiquitin ligases: new structures, new insights, new questions” Biochem. 2014, 458:421; and Wang et al. “Roles of F-box proteins in cancer” Nat. Rev. Cancer 2014, 14:233.

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins. See e.g., Crews, Chem. & Biol. 2010, 17(6):551; Schneekloth and Crews, ChemBioChem 2005, 6(1):40.

An ongoing need exists in the art for effective treatments for disease, especially hyperplasia and cancers. However, non-specific effects, and the inability to target and modulate certain classes of proteins altogether, such as transcription factors, remain as obstacles to the development of effective anti-cancer agents. As such, small molecule therapeutic agents that leverage UPP mediated protein degradation to target cancer-associated proteins such as one or more SWI/SNF-related matrix-associated actin-dependent regulator of chromatin subfamily A (“SMARCA”) and/or polybromo-1 (“PB1”) protein hold promise as therapeutic agents. Accordingly, there remains a need to find compounds that are SMARCA degraders useful as therapeutic agents.

### SUMMARY OF THE INVENTION

The present disclosure relates to novel compounds, which function to recruit one or more SMARCA2, SMARCA4, or PB1 protein to E3 ubiquitin ligases for degradation or directly facilitate ubiquitination for degradation, and methods of preparation and uses thereof. In particular, the present disclosure provides bifunctional compounds, which find utility as modulators of targeted ubiquitination of SMARCA and/or PB1 proteins, which are then degraded and/or otherwise inhibited by the bifunctional compounds as described herein. An advantage of the compounds provided herein is that a broad range of pharmacological activities is possible, consistent with the degradation/inhibition of SMARCA and/or PB1 proteins. In addition, the description provides methods of using an amount of the compounds as described herein for the treatment or amelioration of a disease condition, such as cancer, e.g., lung cancer.

The present application further relates to targeted degradation of SMARCA and/or PB1 proteins through the use of bifunctional molecules, including bifunctional molecules that link a VHL or cereblon-binding moiety to a ligand that binds SMARCA and/or PB1 proteins.

It has now been found that compounds of this disclosure, and pharmaceutically acceptable compositions thereof, are effective for the modulation of targeted ubiquitination. Such compounds have the general formula I:

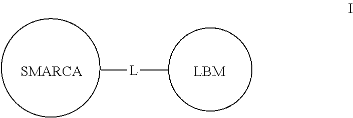

or a pharmaceutically acceptable salt thereof, wherein each variable is as defined and described herein.

It has now been found that compounds of this invention, and pharmaceutically acceptable compositions thereof, are effective as degraders of SMARCA and/or PB1 proteins. Such compounds have the formula I-a:

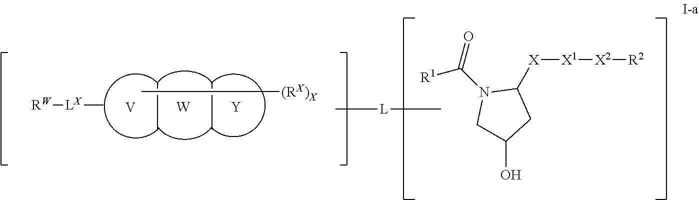

or a pharmaceutically acceptable salt thereof, wherein each variable is as defined and described herein.

Compounds of the present disclosure, and pharmaceutically acceptable compositions thereof, are useful for treating a variety of diseases, disorders or conditions, associated with regulation of signaling pathways implicating SMARCA and/or PB1 proteins. Such diseases, disorders, or conditions include those described herein.

Compounds provided by this disclosure are also useful for the study of SMARCA and/or PB1 proteins in biological and pathological phenomena; the study of intracellular signal transduction pathways occurring in bodily tissues; and the comparative evaluation of new SMARCA and/or PB1 inhibitors or SMARCA and/or PB1 degraders or other regulators of cell cycling, metastasis, angiogenesis, and immune cell evasion, in vitro or in vivo.

### DETAILED DESCRIPTION OF CERTAIN EMBODIMENTS

### 1. General Description of Certain Embodiments of the Invention

Compounds of the present disclosure, and compositions thereof, are useful as degraders and/or inhibitors of SMARCA and/or PB1 proteins. In some embodiments, a provided compound degrades and/or inhibits one or more of SMARCA2, SMARCA4, and PB1 protein.

In certain embodiments, the present invention provides a compound of formula I:

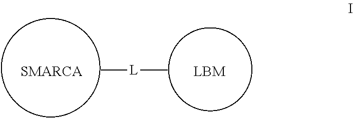

or a pharmaceutically acceptable salt thereof, wherein:
SMARCA is a protein binding moiety capable of binding to one or more of SMARCA2, SMARCA4, and PB1;
L is a bivalent moiety that connects SMARCA to LBM; and
LBM is an E3 ubiquitin ligase binding moiety.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. A carbocyclic group may be monocyclic, bicyclic, bridged bicyclic, or spirocyclic. Without limitation, a carbocyclic group may contain 1-2 oxo groups. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

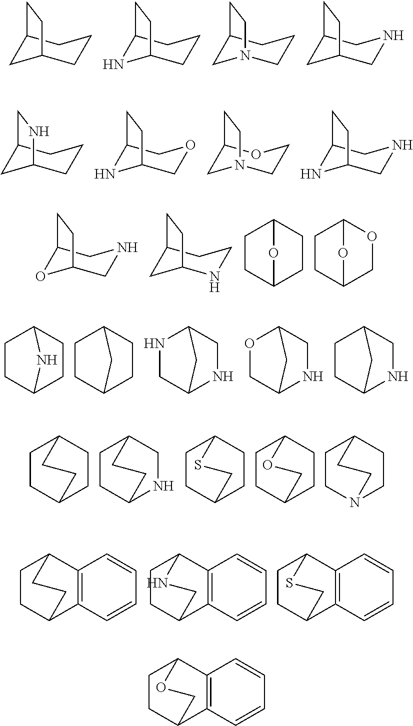

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈(or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

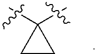

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or *NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclic group may be monocyclic, bicyclic, bridged bicyclic, or spirocyclic. Without limitation, a heterocyclic group may contain 1-2 oxo groups. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)_{0- 4}OP(O)R^{∘}₂; —(CH₂)₀₋₄OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{•} include=O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

As used herein, the term “and/or” is used in this disclosure to mean either “and” or “or” unless indicated otherwise.

As used herein, the term “inhibitor” is defined as a compound that binds to and/or inhibits a SMARCA and/or PB1protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a monovalent or bifunctional compound that binds to and/or inhibits a SMARCA and/or PB1 protein and optionally an E3 ligase with measurable affinity resulting in the ubiquitination and subsequent degradation of the SMARCA and/or PB1 protein. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a compound without an appended E3 ligase.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a SMARCA and/or PB1 protein activity between a sample comprising a compound of the present invention, or composition thereof, and a SMARCA and/or PB1 protein, and an equivalent sample comprising a SMARCA and/or PB1 protein, in the absence of said compound, or composition thereof.

### 3. Description of Exemplary Embodiments

As described above, in certain embodiments, the present disclosure provides a compound of formula I:

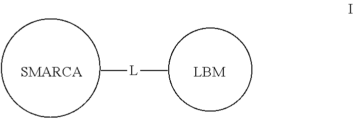

or a pharmaceutically acceptable salt thereof, wherein:
SMARCA is a protein binding moiety capable of binding to one or more of SMARCA2, SMARCA4, and PB1;
L is a bivalent moiety that connects SMARCA to LBM; and
LBM is an E3 ubiquitin ligase binding moiety, such as von Hippel-Lindau (VHL) or cereblon (CRBN).

As defined above and described herein, SMARCA is a SMARCA binding moiety capable of binding to one or more of SMARCA2, SMARCA4, and PB1. In some embodiments, SMARCA is a SMARCA binding moiety capable of degrading one or more of SMARCA2, SMARCA4, and PB1.

In some embodiments SMARCA is a binding moiety capable of selectively binding and degrading SMARCA2 over SMARCA4 and/or PB1. In some embodiments, SMARCA is a binding moiety capable of selectively binding and degrading SMARCA4 over SMARCA2 and/or PB1. In some embodiments, SMARCA is a binding moiety capable of selectively binding and degrading PB1 over SMARCA2 and/or SMARCA4. In some embodiments, SMARCA is a binding moiety capable of selectively binding and degrading SMARCA2 and SMARCA4 over PB1. In some embodiments, SMARCA is a binding moiety capable of selectively binding and degrading SMARCA2 and PB1 over SMARCA4. In some embodiments, SMARCA is a binding moiety capable of selectively binding and degrading SMARCA4 and PB1 over SMARCA2. In some embodiments, SMARCA is a binding moiety capable of binding and degrading SMARCA2, SMARCA4, and PB1.

In certain embodiments, the present invention provides a compound of formula I-a:

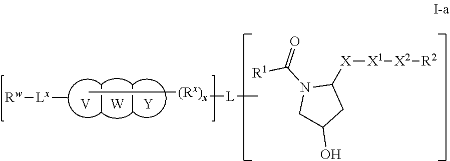

or a pharmaceutically acceptable salt thereof, wherein:
each of Ring V, Ring W, and Ring Y is independently a fused ring selected from 6-membered aryl, 5-6 membered heteroaryl containing 1-4 heteroatoms independently selected from nitrogen, oxygen, and sulfur, and a 4-9 membered saturated or partially unsaturated monocyclic, bicyclic, or bridged bicyclic carbocyclyl or heterocyclyl with 1-4 heteroatoms independently selected from nitrogen, oxygen, and sulfur;
R^{w} is selected from

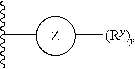

or hydrogen;
Ring Z is phenyl, a 5-7 membered saturated or partially unsaturated carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur;
each of R^{x} and R^{y} is independently hydrogen, R^{z}, halogen, —CN, —NO₂, —OR, —SR, —N(R)₂, —Si(R)₃, —S(O)₂R, —S(O)₂N(R)₂, —S(O)R, —CF(R)₂, —CF₂R, —CF₃, —C(O)R, —C(O)OR, —C(O)N(R)₂, —C(O)NROR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)2, —NRC(O)OR, —NRC(O)R, —NRC(O)N(R)₂, —NRS(O)₂R, —NP(O)R₂, —NRP(O)(OR)₂, —NRP(O)(OR)N(R)₂, —NRP(O)(N(R)₂)2, or —NRS(O)₂R; or
   two R^{x} groups or two R^{y} groups are optionally taken together to form an optionally substituted 5-7 membered partially unsaturated or aryl fused ring having 0-2 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form an optionally substituted 3-7 membered saturated or partially unsaturated carbocyclic ring or an optionally substituted 3-7 membered saturated, partially unsaturated, or heteroaryl ring having 0-3 heteroatoms, in addition to the atom to which they are attached, independently selected from nitrogen, oxygen, and sulfur;
each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CFR—, —CF₂—, —NR—, —S—, —S(O)₂— or —CR═CR—; and
x is 0, 1, 2, 3, 4, 5, 6, 7, 8, 9, 10, 11, 12, 13, 14, 15, or 16;
y is 0, 1, 2, 4, or 5;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

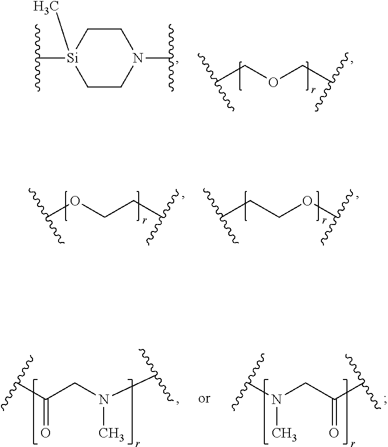

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a covalent bond or bivalent group selected from —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, and a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{z}, —C(R)₂R^{z}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R² is hydrogen, halogen, —CN,

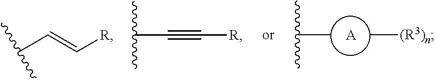

Ring A is a ring selected from phenyl, a 5-6 membered heteroaryl containing 1-4 heteroatoms independently selected from nitrogen, oxygen, and sulfur, and a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, R^{z}, halogen, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)2, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)2, or —N(R)SO₂R; or
   two R³ groups are optionally taken together to form an optionally substituted 5-7 membered partially unsaturated or aryl fused ring having 0-2 heteroatoms independently selected from nitrogen, oxygen, and sulfur;
n is 0, 1, 2, 4, or 5.

As described herein, a structure depicted as

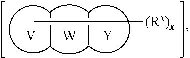

includes for example, structures

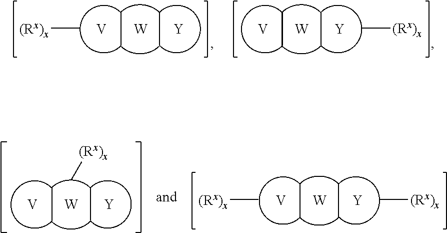

As described herein, wherein a formula is depicted using square brackets, e.g.,

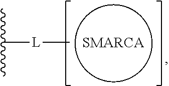

L is attached to a modifiable carbon, oxygen, or nitrogen atom within SMARCA including substitution or replacement of a defined group in SMARCA.

As described herein, wherein a formula is depicted using square brackets, e.g.,

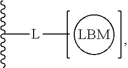

L is attached to a modifiable carbon, oxygen, or nitrogen atom within LBM including substitution or replacement of a defined group in LBM.

As described herein, formula I is formula I-a where SMARCA is

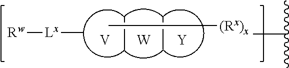

### and LBM is

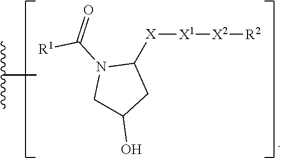

As defined above and described herein, in some embodiments, each of Ring V, Ring W, and Ring Y is independently a fused ring selected from 6-membered aryl, 5-6 membered heteroaryl containing 1-4 heteroatoms independently selected from nitrogen, oxygen, and sulfur, and a 4-9 membered saturated or partially unsaturated monocyclic, bicyclic, or bridged bicyclic carbocyclyl or heterocyclyl with 1-4 heteroatoms independently selected from nitrogen, oxygen, and sulfur.

In some embodiments, one or more of Ring V, Ring W, and Ring Y is a fused 6-membered aryl. In some embodiments, one or more of Ring V, Ring W, and Ring Y is a fused 5-6 membered heteroaryl containing 1-4 heteroatoms independently selected from nitrogen, oxygen, and sulfur. In some embodiments, one or more of Ring V, Ring W, and Ring Y is a fused 4-9 membered saturated or partially unsaturated monocyclic, bicyclic, or bridged bicyclic carbocyclyl. In some embodiments, one or more of Ring V, Ring W, and Ring Y is a fused 4-9 membered saturated or partially unsaturated monocyclic, bicyclic, or bridged bicyclic heterocyclyl with 1-3 heteroatoms independently selected from nitrogen, oxygen, and sulfur.

In some embodiments, Ring V is

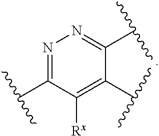

In some embodiments, Ring W is

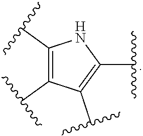 [SMILES: *c1[nH]c(*)c(*)c1*]

In some embodiments, Ring W is

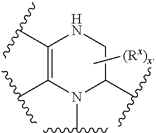

In some embodiments, Ring Y is

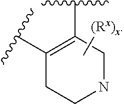

In some embodiments, Ring Y is

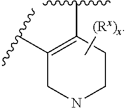

In some embodiments, Ring Y is

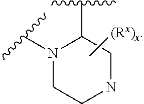

In some embodiments, Ring Y is

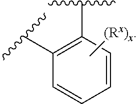

In some embodiments, Ring V, Ring W, and Ring Y are selected from those depicted in Table 1, below.

As defined above and described herein, in some embodiments, R^{w} is selected from

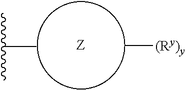

or hydrogen.

In some embodiments, R^{w} is

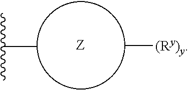

In some embodiments, R^{w} is hydrogen.

In some embodiments, R^{w} is selected from those depicted in Table 1, below.

As defined above and described herein, in some embodiments, Ring Z is phenyl, a 5-7 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur.

In some embodiments, Ring Z is phenyl. In some embodiments, Ring Z is a 5-7 membered saturated or partially unsaturated carbocyclic or heterocyclic ring having 1-3 heteroatoms independently selected from nitrogen, oxygen, and sulfur. In some embodiments, Ring Z is a 5-6 membered heteroaryl ring having 1-4 heteroatoms independently selected from nitrogen, oxygen, and sulfur.

In some embodiments, Ring Z is selected from those depicted in Table 1, below.

As defined above and described herein, in some embodiments, each of R^{x} and R^{y} is independently hydrogen, R^{z}, halogen, —CN, —NO₂, —OR, —SR, —N(R)₂, —Si(R)₃, —S(O)₂R, —S(O)₂N(R)₂, —S(O)R, —CF(R)₂, —CF₂R, —CF₃, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)(R)₂, —OP(O)(OR)₂, —OP(O)(OR)N(R)₂, —OP(O)(N(R)₂)₂, —N(R)C(O)OR, —N(R)C(O)R, —N(R)C(O)N(R)₂, —N(R)S(O)₂R, —NP(O)(R)₂, —N(R)P(O)(OR)₂, —N(R)P(O)(OR)N(R)₂, —N(R)P(O)(N(R)₂)₂, or —N(R)S(O)₂R, or two R^{x} groups or two R^{y} groups are optionally taken together to form an optionally substituted 5-7 membered partially unsaturated or aryl fused ring having 0-2 heteroatoms independently selected from nitrogen, oxygen, and sulfur.

In some embodiments, R^{x} and/or R is hydrogen. In some embodiments, R^{x} and/or R^{y} is R^{z}. In some embodiments, R^{x} and/or R^{y} is halogen. In some embodiments, R^{x} and/or R^{y} is —CN. In some embodiments, R^{x} and/or R^{y} is —NO₂. In some embodiments, R^{x} and/or R^{y} is —OR. In some embodiments, R^{x} and/or R^{y} is —SR. In some embodiments, R^{x} and/or R^{y} is —N(R)₂. In some embodiments, R^{x} and/or R^{y} is —Si(R)₃. In some embodiments, R^{x} and/or R^{y} is —S(O)₂R. In some embodiments, R^{x} and/or R^{y} is —S(O)₂NR₂. In some embodiments, R^{x} and/or R^{y} is —S(O)R. In some embodiments, R^{x} and/or R^{y} is —CF(R)₂. In some embodiments, R^{x} and/or R^{y} is —CF₂R. In some embodiments, R^{x} and/or R^{y} is —CF₃. In some embodiments, R^{x} and/or R^{y} is —C(O)R. In some embodiments, R^{x} and/or R^{y} is —C(O)OR. In some embodiments, R^{x} and/or R^{y} is —C(O)NR₂. In some embodiments, R^{x} and/or R^{y} is —C(O)N(R)OR. In some embodiments, R^{x} and/or R^{y} is —C(R)₂N(R)C(O)R. In some embodiments, R^{x} and/or R^{y} is —C(R)₂N(R)C(O)N(R)₂. In some embodiments, R^{x} and/or R^{y} is —OC(O)R. In some embodiments, R^{x} and/or R^{y} is —OC(O)N(R)₂. In some embodiments, R^{x} and/or R^{y} is —OP(O)(R)₂. In some embodiments, R^{x} and/or R^{y} is —OP(O)(OR)₂. In some embodiments, R^{x} and/or R^{y} is —OP(O)(OR)N(R)₂. In some embodiments, R^{x} and/or R is —OP(O)(N(R)₂)₂. In some embodiments, R^{x} and/or R is —N(R)C(O)OR. In some embodiments, R^{x} and/or R^{y} is —N(R)C(O)R. In some embodiments, R^{x} and/or R^{y} is —N(R)C(O)N(R)₂. In some embodiments, R^{x} and/or R^{y} is —N(R)S(O)₂R. In some embodiments, R^{x} and/or R^{y} is —NP(O)(R)₂. In some embodiments, R and/or R^{y} is —N(R)P(O)(OR)₂. In some embodiments, R^{x} and/or R^{y} is —N(R)P(O)(OR)N(R)₂. In some embodiments, R^{x} and/or R^{y} is —N(R)P(O)(N(R)₂)₂. In some embodiments, R^{x} and/or R^{y} is —N(R)S(O)₂R. In some embodiments, two R^{x} groups or two R^{y} groups are optionally taken together to form an optionally substituted 5-7 membered partially unsaturated or aryl fused ring having 0-2 heteroatoms independently selected from nitrogen, oxygen, and sulfur.

In some embodiments, R^{y} is —OH.

In some embodiments, R is C₁₋₆ alkyl (e.g., methyl, ethyl, isopropyl). In some embodiments, R^{x} is methyl. In some embodiments, R is —CHF₂. In some embodiments, R^{x} is —CH₂OH.

In some embodiments, R^{x} and R^{y} are selected from those depicted in Table 1, below.

As defined above and described herein, in some embodiments, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are taken together with their intervening atoms to form a 4-7 membered saturated, partially unsaturated, or heteroaryl ring having 0-3 heteroatoms, in addition to the atom to which they are attached, independently selected from nitrogen, oxygen, and sulfur.

In some embodiments, R is hydrogen. In some embodiments, R is an optionally substituted C₁₋₆ aliphatic. In some embodiments, R is C₁₋₆ alkyl (e.g., methyl, ethyl, isopropyl). In some embodiments, R is C₁₋₆ haloalkyl (e.g., —CF₃, —CHF₂). In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are taken together with their intervening atoms to form an optionally substituted 4-7 membered saturated, partially unsaturated, or heteroaryl ring having 0-3 heteroatoms, in addition to the atom to which they are attached, independently selected from nitrogen, oxygen, and sulfur.

In some embodiments, R is selected from those depicted in Table 1, below.

As defined above and described herein, in some embodiments, each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{z} is an optionally substituted C₁₋₆ aliphatic. In some embodiments, R^{z} is C₁₋₆ alkyl (e.g., methyl, ethyl, isopropyl). In some embodiments, R^{z} is C₁₋₆ haloalkyl (e.g., —CF₃, —CHF₂). In some embodiments, R^{z} is an optionally substituted phenyl. In some embodiments, R^{z} is an optionally substituted 4-7 membered saturated or partially unsaturated heterocyclic ring having 1-2 heteroatoms independently selected from nitrogen, oxygen, and sulfur. In some embodiments, R^{z} is an optionally substituted 5-6 membered heteroaryl ring having 1-4 heteroatoms independently selected from nitrogen, oxygen, and sulfur.

In some embodiments, R^{z} is selected from those depicted in Table 1, below.

As defined above and described herein, in some embodiments, LU is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CF(R)—, —C(F)₂—, —N(R)—, —S—, —S(O)₂— or —CR═CR—.

In some embodiments, LU is a covalent bond. In some embodiments, L^{x} is a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CF(R)—, —C(F)₂—, —N(R)—, —S—, —S(O)₂- or —CR═CR—.

In some embodiments, L^{x} is selected from those depicted in Table 1, below.

As defined above and described herein, in some embodiments, x is 0, 1, 2, 3, 4, 5, 6, 7, 8, 9, 10, 11, 12, 13, 14, 15, or 16.

In some embodiments, x is 0. In some embodiments, x is 1. In some embodiments, x is 2. In some embodiments, x is 3. In some embodiments, x is 4. In some embodiments, x is 5. In some embodiments, x is 6. In some embodiments, x is 7. In some embodiments, x is 8. In some embodiments, x is 9. In some embodiments, x is 10. In some embodiments, x is 11. In some embodiments, x is 12. In some embodiments, x is 13. In some embodiments, x is 14. In some embodiments, x is 15. In some embodiments, x is 16.

In some embodiments, x is selected from those depicted in Table 1, below.

As defined above and described herein, in some embodiments, y is 0, 1, 2, 4, or 5.

In some embodiments, y is 0. In some embodiments, y is 1. In some embodiments, y is 2. In some embodiments, y is 3. In some embodiments, y is 4. In some embodiments, y is 5.

In some embodiments, y is selected from those depicted in Table 1, below.

As defined above and described herein, in some embodiments, L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

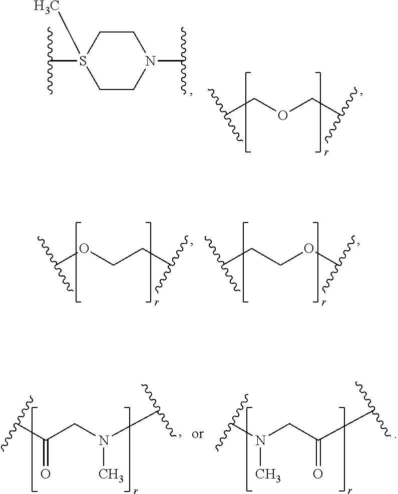

In some embodiments, L is a covalent bond. In some embodiments, L is bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

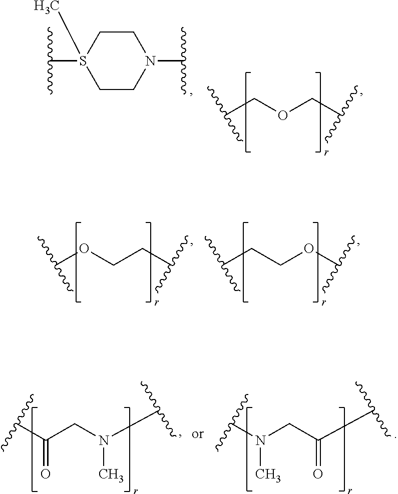

As defined above and described herein, in some embodiments, each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur.

In some embodiments, -Cy- is an optionally substituted phenylenyl. In some embodiments, -Cy- is an optionally substituted 8-10 membered bicyclic arylenyl. In some embodiments, -Cy- is an optionally substituted 4-7 membered saturated or partially unsaturated carbocyclylenyl. In some embodiments, -Cy- is an optionally substituted 4-11 membered saturated or partially unsaturated spiro carbocyclylenyl. In some embodiments, -Cy- is an optionally substituted 8-10 membered bicyclic saturated or partially unsaturated carbocyclylenyl. In some embodiments, -Cy- is an optionally substituted 4-7 membered saturated or partially unsaturated heterocyclylenyl having 1-2 heteroatoms independently selected from nitrogen, oxygen, and sulfur. In some embodiments, -Cy- is an optionally substituted 4-11 membered saturated or partially unsaturated spiro heterocyclylenyl having 1-2 heteroatoms independently selected from nitrogen, oxygen, and sulfur. In some embodiments, -Cy- is an optionally substituted 8-10 membered bicyclic saturated or partially unsaturated heterocyclylenyl having 1-2 heteroatoms independently selected from nitrogen, oxygen, and sulfur. In some embodiments, -Cy- is an optionally substituted 5-6 membered heteroarylenyl having 1-4 heteroatoms independently selected from nitrogen, oxygen, and sulfur. In some embodiments, -Cy- is an optionally substituted 8-10 membered bicyclic heteroarylenyl having 1-5 heteroatoms independently selected from nitrogen, oxygen, and sulfur.

In some embodiments, -Cy- is

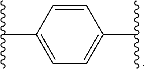 [SMILES: *c1ccc(*)cc1]

In some embodiments, -Cy- is

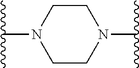 [SMILES: *N1CCN(*)CC1]

In some embodiments, -Cy- is

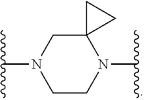 [SMILES: *N1CCN(*)C2(CC2)C1]

In some embodiments, -Cy- is

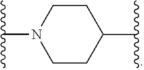 [SMILES: *C1CCN(*)CC1]

In some embodiments, -Cy- is

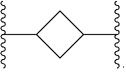 [SMILES: *C1CC(*)C1]

In some embodiments, -Cy- is

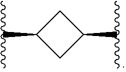 [SMILES: *[C@H]1C[C@@H](*)C1]

In some embodiments, -Cy- is

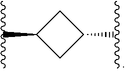 [SMILES: *[C@H]1C[C@H](*)C1]

In some embodiments, -Cy- is

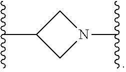 [SMILES: *C1CN(*)C1]

In some embodiments, -Cy- is

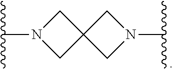 [SMILES: *N1CC2(C1)CN(*)C2]

In some embodiments, -Cy- is

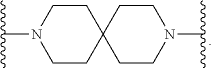 [SMILES: *N1CCC2(CC1)CCN(*)CC2]

In some embodiments, -Cy- is

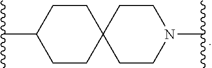 [SMILES: *C1CCC2(CC1)CCN(*)CC2]

In some embodiments, -Cy- is

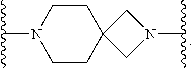 [SMILES: *N1CCC2(CC1)CN(*)C2]

In some embodiments, -Cy- is

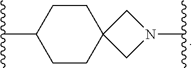 [SMILES: *C1CCC2(CC1)CN(*)C2]

In some embodiments, -Cy- is

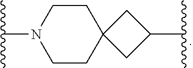 [SMILES: *C1CC2(CCN(*)CC2)C1]

In some embodiments, -Cy- is

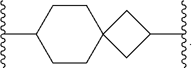 [SMILES: *C1CCC2(CC1)CC(*)C2]

In some embodiments, -Cy- is

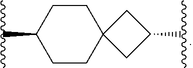 [SMILES: *[C@H]1CCC2(CC1)C[C@H](*)C2]

In some embodiments, -Cy- is

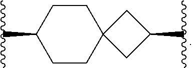 [SMILES: *[C@H]1CCC2(CC1)C[C@H](*)C2]

In some embodiments, -Cy- is

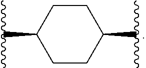 [SMILES: *[C@H]1CC[C@@H](*)CC1]

In some embodiments, -Cy- is

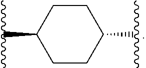 [SMILES: *[C@H]1CC[C@H](*)CC1]

In some embodiments, -Cy- is

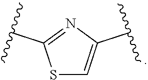 [SMILES: *c1csc(*)n1]

In some embodiments, -Cy- is

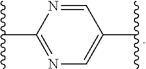 [SMILES: *c1cnc(*)nc1]

In some embodiments, Cy is

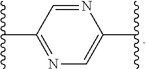 [SMILES: *c1cnc(*)cn1]

In some embodiments, -Cy- is

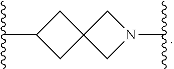 [SMILES: *C1CC2(C1)CN(*)C2]

In some embodiments, -Cy- is

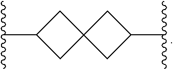 [SMILES: *C1CC2(C1)CC(*)C2]

In some embodiments, -Cy- is

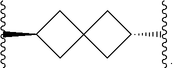 [SMILES: *[C@H]1CC2(C[C@H](*)C2)C1]

In some embodiments, -Cy- is

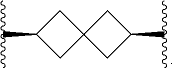 [SMILES: *[C@H]1CC2(C[C@H](*)C2)C1]

In some embodiments, -Cy- is optionally substituted with one or more fluoro atoms.

In some embodiments, -Cy- is selected from those depicted in Table 1, below.

As defined above and described herein, in some embodiments, r is 0, 1, 2, 3, 4, 5, 6, 7, 8, 9, 10, 11, 12, 13, 14, 15, or 16.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1, below.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1, below.

In some embodiments, L is —NR—(C₁₋₁₀ aliphatic)-. In some embodiments, L is —(C₁₋₁₀ aliphatic)-NR—(C₁₋₁₀aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is -Cy-(C₁₋₁₀ aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is -Cy-(C₁₋₁₀ aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀—. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—O—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀-Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is -Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—.

In some embodiments, L is -Cy-Cy-. In some embodiments, L is —(CH₂)₁₋₁₀-Cy-Cy-. In some embodiments, L is -Cy-(CH₂)₁₋₁₀-Cy-.

In some embodiments, L is -Cy-Cy-O—. In some embodiments, L is —(CH₂)₁₋₁₀-Cy-Cy-O—. In some embodiments, L is -Cy-(CH₂)₁₋₁₀-Cy-O—. In some embodiments, L is -Cy-Cy-(CH₂)₁₋₁₀—O—.

In some embodiments, L is -Cy-Cy-CO—. In some embodiments, L is —(CH₂)₁₋₁₀-Cy-Cy-CO—. In some embodiments, L is -Cy-(CH₂)₁₋₁₀-Cy-CO—. In some embodiments, L is -Cy-Cy-(CH₂)₁₋₁₀—CO—.

In some embodiments, L is -Cy-Cy-Cy-O—. In some embodiments, L is -Cy-(CH₂)₁₋₁₀-Cy-Cy-O—. In some embodiments, L is -Cy-Cy-(CH₂)₁₋₁₀-Cy-O—. In some embodiments, L is -Cy-Cy-Cy-(CH₂)₁₋₁₀—O—.

In some embodiments, L is -Cy-Cy-Cy-CO—. In some embodiments, L is -Cy-(CH₂)₁₋₁₀-Cy-Cy-CO—. In some embodiments, L is -Cy-Cy-(CH₂)₁₋₁₀-Cy-CO—. In some embodiments, L is -Cy-Cy-Cy-(CH₂)₁₋₁₀—CO—.

In some embodiments, L is

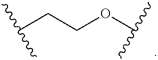 [SMILES: *CCO*]

In some embodiments, L is

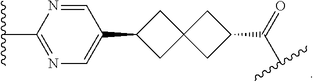 [SMILES: *c1ncc([C@H]2CC3(C[C@H](C(*)=O)C3)C2)cn1]

In some embodiments, L is

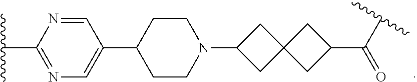 [SMILES: *C(=O)C1CC2(C1)CC(N1CCC(c3cnc(*)nc3)CC1)C2]

In some embodiments, L is N

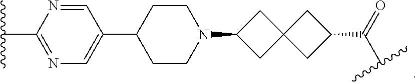 [SMILES: *c1ncc(C2CCN([C@H]3CC4(C[C@H](C(*)=O)C4)C3)CC2)cn1]

In some embodiments, L is

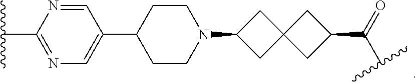 [SMILES: *c1ncc(C2CCN([C@H]3CC4(C[C@H](C(*)=O)C4)C3)CC2)cn1]

In some embodiments, L is

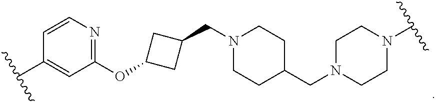 [SMILES: *c1ccnc(O[C@H]2C[C@H](CN3CCC(CN4CCN(*)CC4)CC3)C2)c1]

In some embodiments, L is

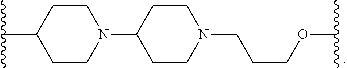 [SMILES: *OCCCN1CCC(N2CCC(*)CC2)CC1]

In some embodiments, L is

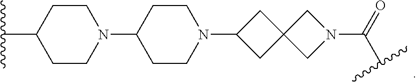 [SMILES: *C(=O)N1CC2(CC(N3CCC(N4CCC(*)CC4)CC3)C2)C1]

In some embodiments, L is

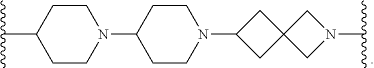 [SMILES: *C1CCN(C2CCN(C3CC4(C3)CN(*)C4)CC2)CC1]

In some embodiments, L is

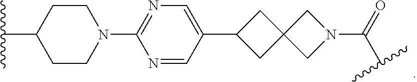 [SMILES: *C(=O)N1CC2(CC(c3cnc(N4CCC(*)CC4)nc3)C2)C1]

In some embodiments, L is

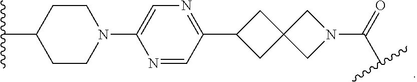 [SMILES: *C(=O)N1CC2(CC(c3cnc(N4CCC(*)CC4)cn3)C2)C1]

In some embodiments, L is

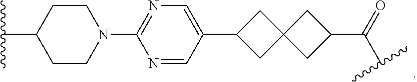 [SMILES: *C(=O)C1CC2(C1)CC(c1cnc(N3CCC(*)CC3)nc1)C2]

In some embodiments, L is

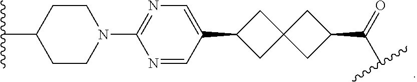 [SMILES: *C(=O)[C@H]1CC2(C1)C[C@H](c1cnc(N3CCC(*)CC3)nc1)C2]

In some embodiments, L is

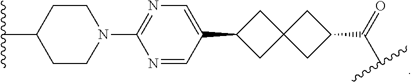 [SMILES: *C(=O)[C@H]1CC2(C1)C[C@H](c1cnc(N3CCC(*)CC3)nc1)C2]

In some embodiments, L is

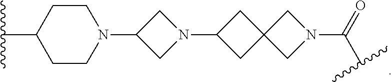 [SMILES: *C(=O)N1CC2(CC(N3CC(N4CCC(*)CC4)C3)C2)C1]

In some embodiments, L is

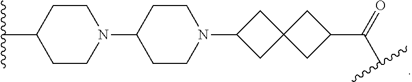 [SMILES: *C(=O)C1CC2(C1)CC(N1CCC(N3CCC(*)CC3)CC1)C2]

In some embodiments, L is

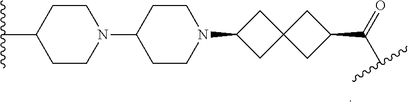 [SMILES: *C(=O)[C@H]1CC2(C1)C[C@H](N1CCC(N3CCC(*)CC3)CC1)C2]

In some embodiments, L is

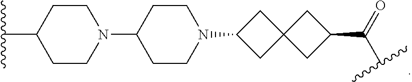 [SMILES: *C(=O)[C@H]1CC2(C1)C[C@H](N1CCC(N3CCC(*)CC3)CC1)C2]

In some embodiments, L is

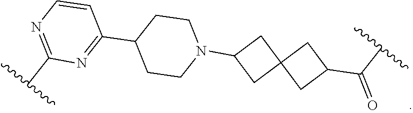 [SMILES: *C(=O)C1CC2(C1)CC(N1CCC(c3ccnc(*)n3)CC1)C2]

In some embodiments, L is

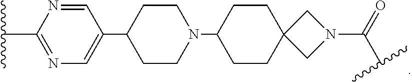 [SMILES: *C(=O)N1CC2(CCC(N3CCC(c4cnc(*)nc4)CC3)CC2)C1]

In some embodiments, L is

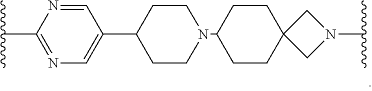 [SMILES: *c1ncc(C2CCN(C3CCC4(CC3)CN(*)C4)CC2)cn1]

In some embodiments, L is

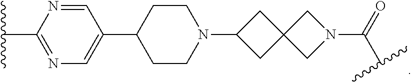 [SMILES: *C(=O)N1CC2(CC(N3CCC(c4cnc(*)nc4)CC3)C2)C1]

In some embodiments, L is

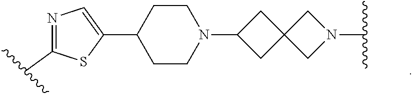 [SMILES: *c1ncc(C2CCN(C3CC4(C3)CN(*)C4)CC2)s1]

In some embodiments, L is

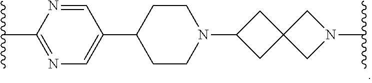 [SMILES: *c1ncc(C2CCN(C3CC4(C3)CN(*)C4)CC2)cn1]

In some embodiments, L is

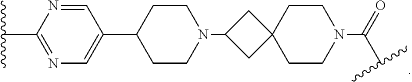 [SMILES: *C(=O)N1CCC2(CC1)CC(N1CCC(c3cnc(*)nc3)CC1)C2]

In some embodiments, L is

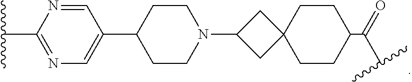 [SMILES: *C(=O)C1CCC2(CC1)CC(N1CCC(c3cnc(*)nc3)CC1)C2]

In some embodiments, L is

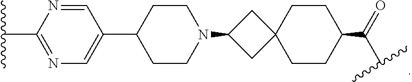 [SMILES: *c1ncc(C2CCN([C@H]3CC4(CC[C@H](C(*)=O)CC4)C3)CC2)cn1]

N In some embodiments, L is

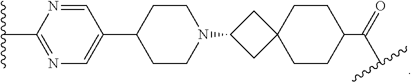

In some embodiments, L is

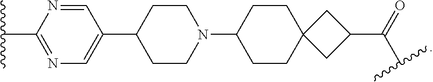 [SMILES: *C(=O)C1CC2(CCC(N3CCC(c4cnc(*)nc4)CC3)CC2)C1]

In some embodiments, L is

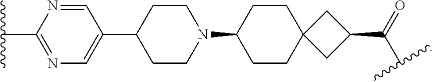 [SMILES: *c1ncc(C2CCN([C@H]3CCC4(CC3)C[C@H](C(*)=O)C4)CC2)cn1]

In some embodiments, L is

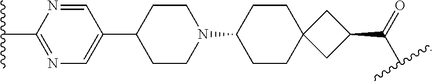 [SMILES: *c1ncc(C2CCN([C@H]3CCC4(CC3)C[C@H](C(*)=O)C4)CC2)cn1]

In some embodiments, L is

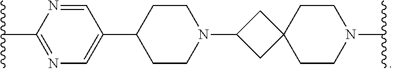 [SMILES: *c1ncc(C2CCN(C3CC4(CCN(*)CC4)C3)CC2)cn1]

In some embodiments, L is

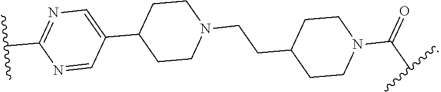 [SMILES: *C(=O)N1CCC(CCN2CCC(c3cnc(*)nc3)CC2)CC1]

In some embodiments, L is

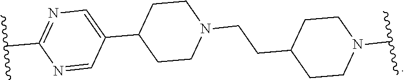 [SMILES: *c1ncc(C2CCN(CCC3CCN(*)CC3)CC2)cn1]

In some embodiments, L is

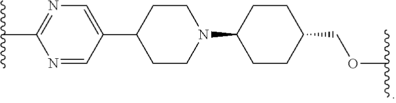 [SMILES: *OC[C@H]1CC[C@H](N2CCC(c3cnc(*)nc3)CC2)CC1]

In some embodiments, L is

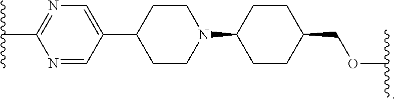 [SMILES: *OC[C@H]1CC[C@@H](N2CCC(c3cnc(*)nc3)CC2)CC1]

In some embodiments, L is

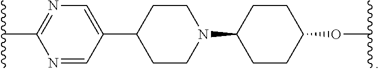 [SMILES: *O[C@H]1CC[C@H](N2CCC(c3cnc(*)nc3)CC2)CC1]

In some embodiments, L is

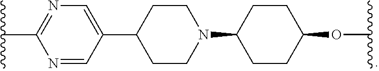 [SMILES: *O[C@H]1CC[C@@H](N2CCC(c3cnc(*)nc3)CC2)CC1]

In some embodiments, L is

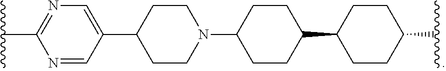 [SMILES: *c1ncc(C2CCN(C3CCC([C@H]4CC[C@H](*)CC4)CC3)CC2)cn1]

In some embodiments, L is

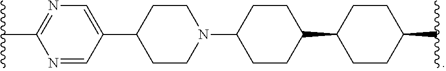 [SMILES: *c1ncc(C2CCN(C3CCC([C@H]4CC[C@@H](*)CC4)CC3)CC2)cn1]

In some embodiments, L is

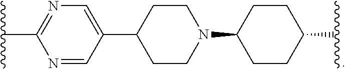 [SMILES: *c1ncc(C2CCN([C@H]3CC[C@H](*)CC3)CC2)cn1]

In some embodiments, L is

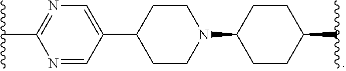 [SMILES: *c1ncc(C2CCN([C@H]3CC[C@@H](*)CC3)CC2)cn1]

In some embodiments, L is

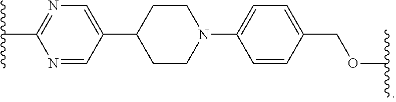 [SMILES: *OCc1ccc(N2CCC(c3cnc(*)nc3)CC2)cc1]

In some embodiments, L is

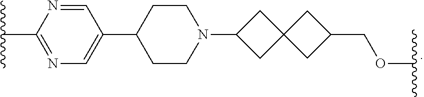 [SMILES: *OCC1CC2(C1)CC(N1CCC(c3cnc(*)nc3)CC1)C2]

In some embodiments, L is

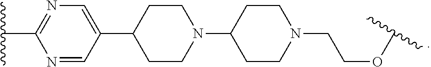 [SMILES: *OCCN1CCC(N2CCC(c3cnc(*)nc3)CC2)CC1]

In some embodiments, L is

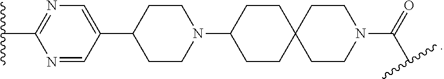 [SMILES: *C(=O)N1CCC2(CCC(N3CCC(c4cnc(*)nc4)CC3)CC2)CC1]

In some embodiments, L is

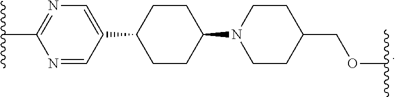 [SMILES: *OCC1CCN([C@H]2CC[C@H](c3cnc(*)nc3)CC2)CC1]

In some embodiments, L is

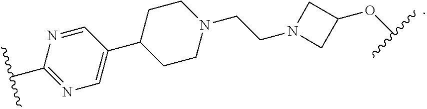 [SMILES: *OC1CN(CCN2CCC(c3cnc(*)nc3)CC2)C1]

In some embodiments, L is

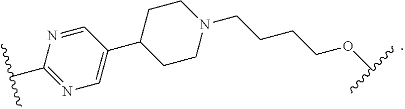 [SMILES: *OCCCCN1CCC(c2cnc(*)nc2)CC1]

In some embodiments, L is

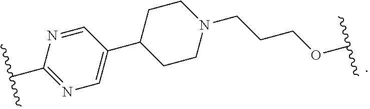 [SMILES: *OCCCN1CCC(c2cnc(*)nc2)CC1]

In some embodiments, L is

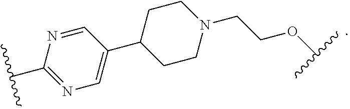 [SMILES: *OCCN1CCC(c2cnc(*)nc2)CC1]

In some embodiments, L is

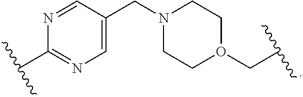

In some embodiments, L is

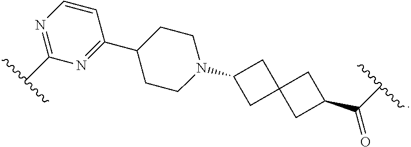 [SMILES: *c1nccc(C2CCN([C@H]3CC4(C[C@H](C(*)=O)C4)C3)CC2)n1]

In some embodiments L is

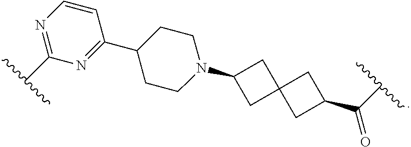 [SMILES: *c1nccc(C2CCN([C@H]3CC4(C[C@H](C(*)=O)C4)C3)CC2)n1]

In some embodiments, L is

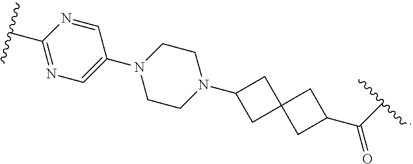 [SMILES: *C(=O)C1CC2(C1)CC(N1CCN(c3cnc(*)nc3)CC1)C2]

In some embodiments, L is

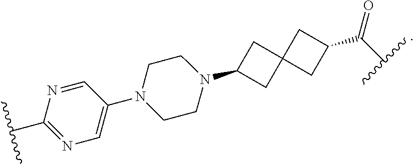 [SMILES: *c1ncc(N2CCN([C@H]3CC4(C[C@H](C(*)=O)C4)C3)CC2)cn1]

In some embodiments, L is

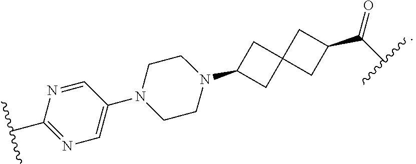 [SMILES: *c1ncc(N2CCN([C@H]3CC4(C[C@H](C(*)=O)C4)C3)CC2)cn1]

In some embodiments, L is

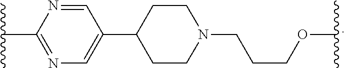 [SMILES: *OCCCN1CCC(c2cnc(*)nc2)CC1]

In some embodiments, L is

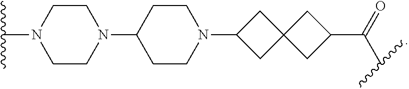 [SMILES: *C(=O)C1CC2(C1)CC(N1CCC(N3CCN(*)CC3)CC1)C2]

In some embodiments, L is

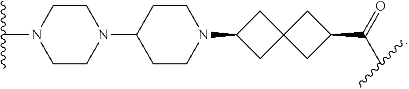 [SMILES: *C(=O)[C@H]1CC2(C1)C[C@H](N1CCC(N3CCN(*)CC3)CC1)C2]

In some embodiments, L is

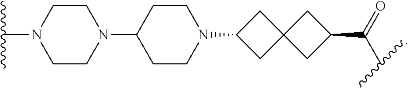 [SMILES: *C(=O)[C@H]1CC2(C1)C[C@H](N1CCC(N3CCN(*)CC3)CC1)C2]

In some embodiments, L is

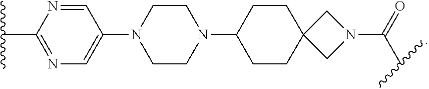 [SMILES: *C(=O)N1CC2(CCC(N3CCN(c4cnc(*)nc4)CC3)CC2)C1]

In some embodiments, L is

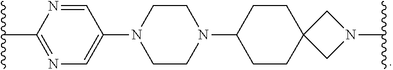 [SMILES: *c1ncc(N2CCN(C3CCC4(CC3)CN(*)C4)CC2)cn1]

In some embodiments, L is

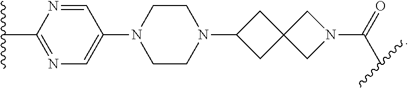 [SMILES: *C(=O)N1CC2(CC(N3CCN(c4cnc(*)nc4)CC3)C2)C1]

In some embodiments, L is

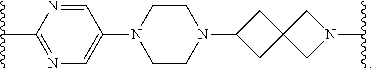 [SMILES: *c1ncc(N2CCN(C3CC4(C3)CN(*)C4)CC2)cn1]

In some embodiments, L is

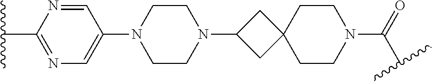 [SMILES: *C(=O)N1CCC2(CC1)CC(N1CCN(c3cnc(*)nc3)CC1)C2]

In some embodiments, L is

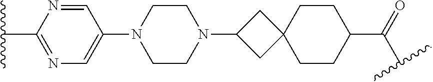 [SMILES: *C(=O)C1CCC2(CC1)CC(N1CCN(c3cnc(*)nc3)CC1)C2]

In some embodiments, L is

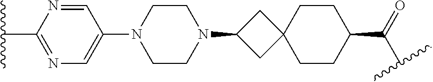 [SMILES: *c1ncc(N2CCN([C@H]3CC4(CC[C@H](C(*)=O)CC4)C3)CC2)cn1]

In some embodiments, L is

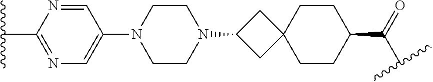 [SMILES: *c1ncc(N2CCN([C@H]3CC4(CC[C@H](C(*)=O)CC4)C3)CC2)cn1]

In some embodiments, L is

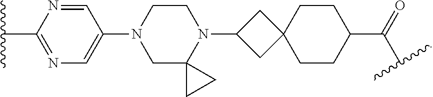 [SMILES: *C(=O)C1CCC2(CC1)CC(N1CCN(c3cnc(*)nc3)CC13CC3)C2]

In some embodiments, L is

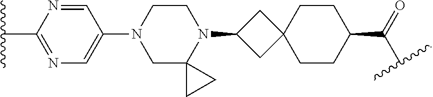 [SMILES: *c1ncc(N2CCN([C@H]3CC4(CC[C@H](C(*)=O)CC4)C3)C3(CC3)C2)cn1]

In some embodiments, L is

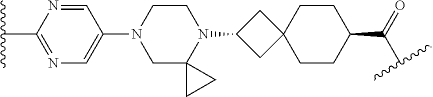 [SMILES: *c1ncc(N2CCN([C@H]3CC4(CC[C@H](C(*)=O)CC4)C3)C3(CC3)C2)cn1]

In some embodiments, L is

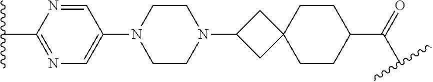 [SMILES: *C(=O)C1CCC2(CC1)CC(N1CCN(c3cnc(*)nc3)CC1)C2]

In some embodiments, L is

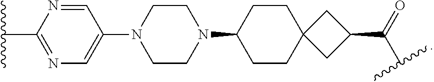 [SMILES: *c1ncc(N2CCN([C@H]3CCC4(CC3)C[C@H](C(*)=O)C4)CC2)cn1]

In some embodiments, L is

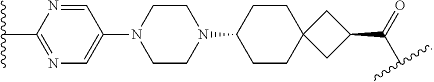 [SMILES: *c1ncc(N2CCN([C@H]3CCC4(CC3)C[C@H](C(*)=O)C4)CC2)cn1]

In some embodiments, L is

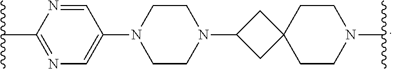 [SMILES: *c1ncc(N2CCN(C3CC4(CCN(*)CC4)C3)CC2)cn1]

In some embodiments, L is

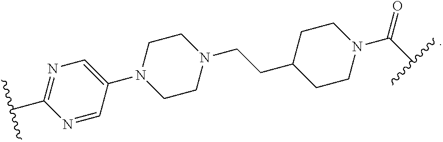 [SMILES: *C(=O)N1CCC(CCN2CCN(c3cnc(*)nc3)CC2)CC1]

In some embodiments, L is

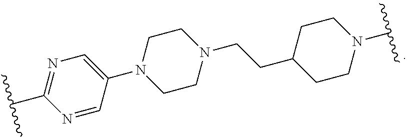 [SMILES: *c1ncc(N2CCN(CCC3CCN(*)CC3)CC2)cn1]

In some embodiments, L is

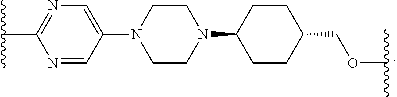 [SMILES: *OC[C@H]1CC[C@H](N2CCN(c3cnc(*)nc3)CC2)CC1]

In some embodiments, L is

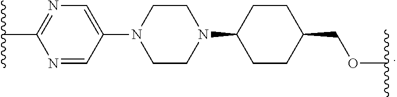 [SMILES: *OC[C@H]1CC[C@@H](N2CCN(c3cnc(*)nc3)CC2)CC1]

In some embodiments, L is

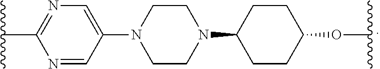 [SMILES: *O[C@H]1CC[C@H](N2CCN(c3cnc(*)nc3)CC2)CC1]

In some embodiments, L is

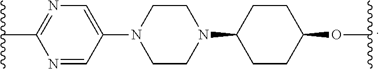 [SMILES: *O[C@H]1CC[C@@H](N2CCN(c3cnc(*)nc3)CC2)CC1]

In some embodiments, L is

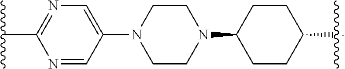 [SMILES: *c1ncc(N2CCN([C@H]3CC[C@H](*)CC3)CC2)cn1]

In some embodiments, L is

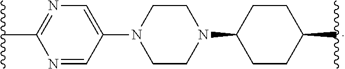 [SMILES: *c1ncc(N2CCN([C@H]3CC[C@@H](*)CC3)CC2)cn1]

In some embodiments, L is

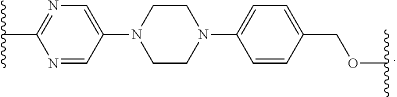 [SMILES: *OCc1ccc(N2CCN(c3cnc(*)nc3)CC2)cc1]

In some embodiments, L is

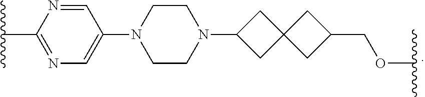 [SMILES: *OCC1CC2(C1)CC(N1CCN(c3cnc(*)nc3)CC1)C2]

In some embodiments, L is

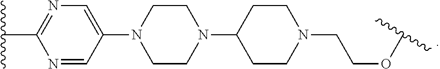 [SMILES: *OCCN1CCC(N2CCN(c3cnc(*)nc3)CC2)CC1]

In some embodiments, L is

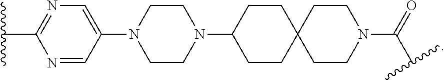 [SMILES: *C(=O)N1CCC2(CCC(N3CCN(c4cnc(*)nc4)CC3)CC2)CC1]

In some embodiments, L is

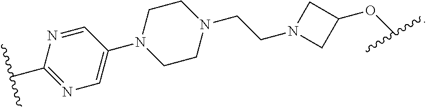 [SMILES: *OC1CN(CCN2CCN(c3cnc(*)nc3)CC2)C1]

In some embodiments, L is

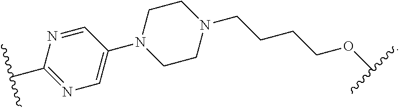 [SMILES: *OCCCCN1CCN(c2cnc(*)nc2)CC1]

In some embodiments, L is

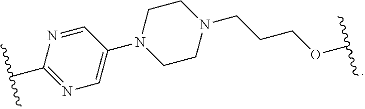 [SMILES: *OCCCN1CCN(c2cnc(*)nc2)CC1]

In some embodiments, L is

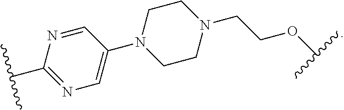 [SMILES: *OCCN1CCN(c2cnc(*)nc2)CC1]

In some embodiments, L is

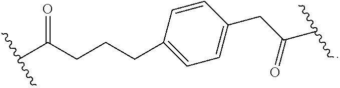 [SMILES: *C(=O)CCCc1ccc(CC(*)=O)cc1]

In some embodiments, L is

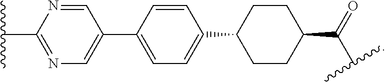 [SMILES: *c1ncc(-c2ccc([C@H]3CC[C@H](C(*)=O)CC3)cc2)cn1]

In some embodiments, L is

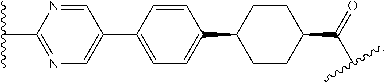 [SMILES: *c1ncc(-c2ccc([C@H]3CC[C@@H](C(*)=O)CC3)cc2)cn1]

In some embodiments, L is

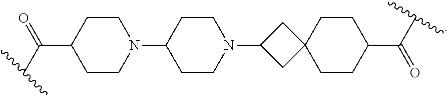 [SMILES: *C(=O)C1CCN(C2CCN(C3CC4(CCC(C(*)=O)CC4)C3)CC2)CC1]

In some embodiments, L is

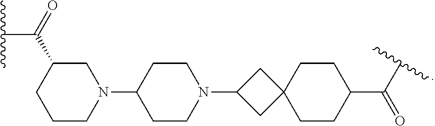 [SMILES: *C(=O)C1CCC2(CC1)CC(N1CCC(N3CCC[C@H](C(*)=O)C3)CC1)C2]

In some embodiments, L is

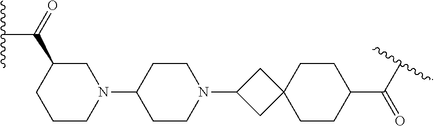 [SMILES: *C(=O)C1CCC2(CC1)CC(N1CCC(N3CCC[C@@H](C(*)=O)C3)CC1)C2]

In some embodiments, L is

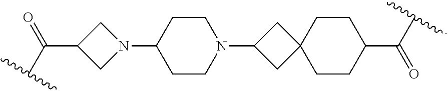 [SMILES: *C(=O)C1CCC2(CC1)CC(N1CCC(N3CC(C(*)=O)C3)CC1)C2]

In some embodiments, L is

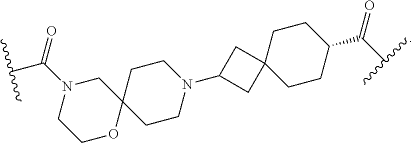

In some embodiments, L is

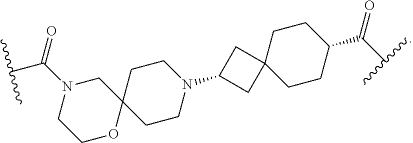 [SMILES: *C(=O)N1CCOC2(CCN([C@H]3CC4(CC[C@H](C(*)=O)CC4)C3)CC2)C1]

In some embodiments, L is

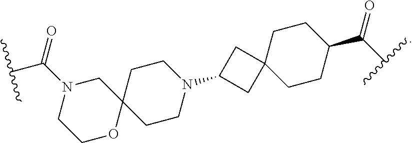 [SMILES: *C(=O)N1CCOC2(CCN([C@H]3CC4(CC[C@H](C(*)=O)CC4)C3)CC2)C1]

In some embodiments, L is

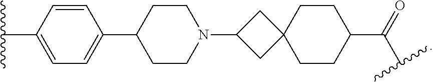 [SMILES: *C(=O)C1CCC2(CC1)CC(N1CCC(c3ccc(*)cc3)CC1)C2]

In some embodiments, L is

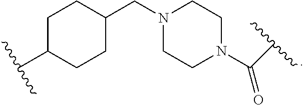 [SMILES: *C(=O)N1CCN(CC2CCC(*)CC2)CC1]

In some embodiments, L is

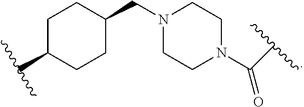 [SMILES: *C(=O)N1CCN(C[C@H]2CC[C@@H](*)CC2)CC1]

In some embodiments, L is

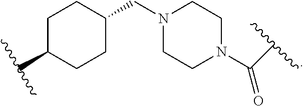 [SMILES: *C(=O)N1CCN(C[C@H]2CC[C@H](*)CC2)CC1]

In some embodiments, L is

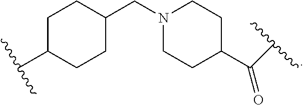 [SMILES: *C(=O)C1CCN(CC2CCC(*)CC2)CC1]

In some embodiments, L is

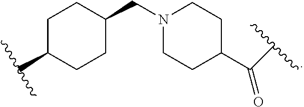 [SMILES: *C(=O)C1CCN(C[C@H]2CC[C@@H](*)CC2)CC1]

In some embodiments, L is

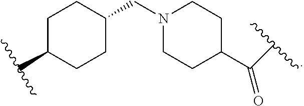 [SMILES: *C(=O)C1CCN(C[C@H]2CC[C@H](*)CC2)CC1]

In some embodiments, L is

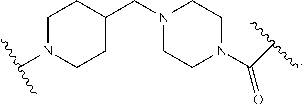 [SMILES: *C(=O)N1CCN(CC2CCN(*)CC2)CC1]

In some embodiments, L is

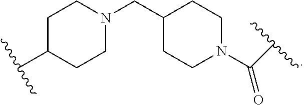 [SMILES: *C(=O)N1CCC(CN2CCC(*)CC2)CC1]

In some embodiments, L is

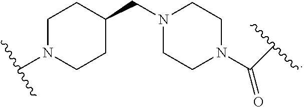

In some embodiments, L is

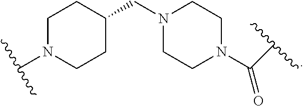

In some embodiments, L is

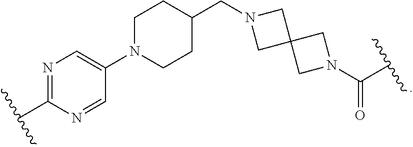 [SMILES: *C(=O)N1CC2(CN(CC3CCN(c4cnc(*)nc4)CC3)C2)C1]

In some embodiments, L is

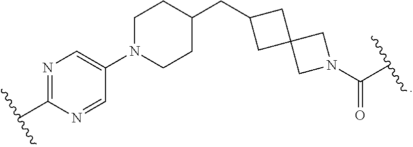 [SMILES: *C(=O)N1CC2(CC(CC3CCN(c4cnc(*)nc4)CC3)C2)C1]

In some embodiments, L is

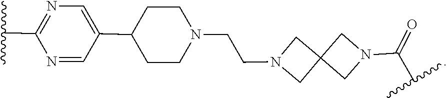 [SMILES: *C(=O)N1CC2(CN(CCN3CCC(c4cnc(*)nc4)CC3)C2)C1]

In some embodiments, L is

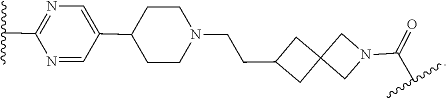 [SMILES: *C(=O)N1CC2(CC(CCN3CCC(c4cnc(*)nc4)CC3)C2)C1]

In some embodiments, L is

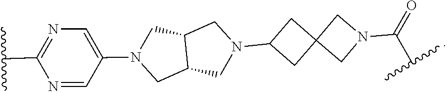 [SMILES: *C(=O)N1CC2(CC(N3C[C@H]4CN(c5cnc(*)nc5)C[C@H]4C3)C2)C1]

In some embodiments, L is

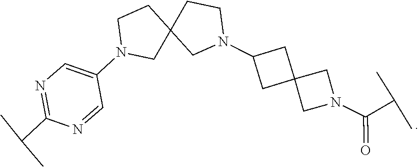 [SMILES: CC(C)C(=O)N1CC2(CC(N3CCC4(CCN(c5cnc(C(C)C)nc5)C4)C3)C2)C1]

In some embodiments, L is

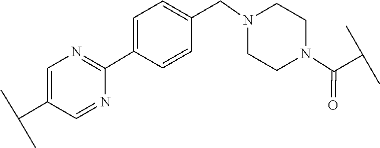 [SMILES: CC(C)C(=O)N1CCN(Cc2ccc(-c3ncc(C(C)C)cn3)cc2)CC1]

In some embodiments, L is

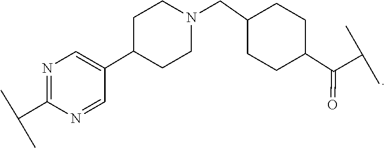 [SMILES: CC(C)C(=O)C1CCC(CN2CCC(c3cnc(C(C)C)nc3)CC2)CC1]

In some embodiments, L is

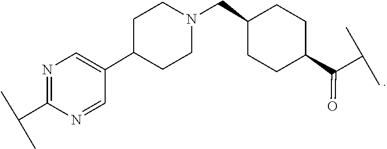 [SMILES: CC(C)c1ncc(C2CCN(C[C@H]3CC[C@@H](C(=O)C(C)C)CC3)CC2)cn1]

In some embodiments, L is

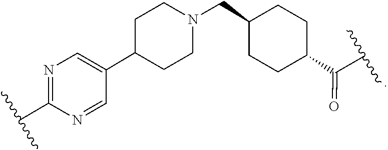 [SMILES: *c1ncc(C2CCN(C[C@H]3CC[C@H](C(*)=O)CC3)CC2)cn1]

In some embodiments, L is

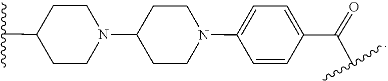 [SMILES: *C(=O)c1ccc(N2CCC(N3CCC(*)CC3)CC2)cc1]

In some embodiments, L is

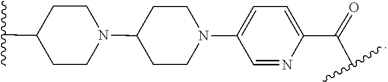 [SMILES: *C(=O)c1ccc(N2CCC(N3CCC(*)CC3)CC2)cn1]

In some embodiments, L is

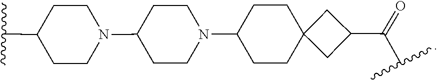 [SMILES: *C(=O)C1CC2(CCC(N3CCC(N4CCC(*)CC4)CC3)CC2)C1]

In some embodiments, L is

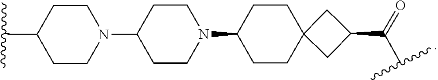 [SMILES: *C(=O)[C@H]1CC2(CC[C@H](N3CCC(N4CCC(*)CC4)CC3)CC2)C1]

In some embodiments, L is

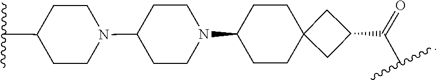 [SMILES: *C(=O)[C@H]1CC2(CC[C@H](N3CCC(N4CCC(*)CC4)CC3)CC2)C1]

In some embodiments, L is

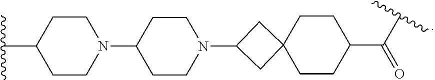 [SMILES: *C(=O)C1CCC2(CC1)CC(N1CCC(N3CCC(*)CC3)CC1)C2]

In some embodiments, L is

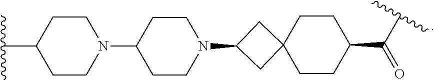 [SMILES: *C(=O)[C@H]1CCC2(CC1)C[C@H](N1CCC(N3CCC(*)CC3)CC1)C2]

In some embodiments, L is

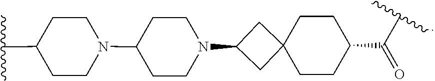 [SMILES: *C(=O)[C@H]1CCC2(CC1)C[C@H](N1CCC(N3CCC(*)CC3)CC1)C2]

In some embodiments, L is

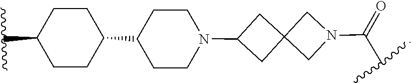 [SMILES: *C(=O)N1CC2(CC(N3CCC([C@H]4CC[C@H](*)CC4)CC3)C2)C1]

In some embodiments, L is

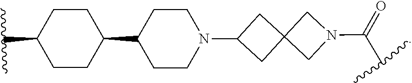 [SMILES: *C(=O)N1CC2(CC(N3CCC([C@H]4CC[C@@H](*)CC4)CC3)C2)C1]

In some embodiments, L is

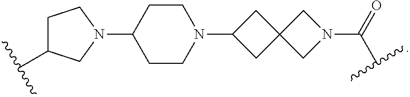 [SMILES: *C(=O)N1CC2(CC(N3CCC(N4CCC(*)C4)CC3)C2)C1]

In some embodiments, L is

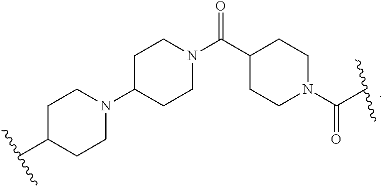 [SMILES: *C(=O)N1CCC(C(=O)N2CCC(N3CCC(*)CC3)CC2)CC1]

In some embodiments, L is

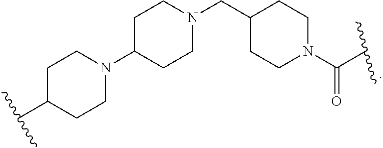 [SMILES: *C(=O)N1CCC(CN2CCC(N3CCC(*)CC3)CC2)CC1]

In some embodiments, L is

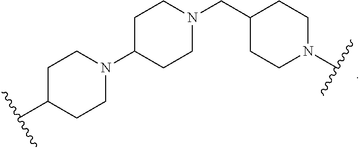 [SMILES: *C1CCN(C2CCN(CC3CCN(*)CC3)CC2)CC1]

In some embodiments, L is

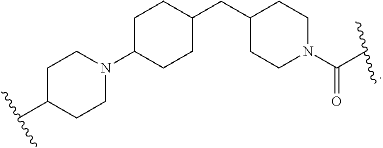 [SMILES: *C(=O)N1CCC(CC2CCC(N3CCC(*)CC3)CC2)CC1]

In some embodiments, L is

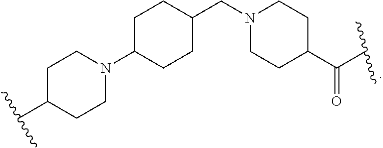 [SMILES: *C(=O)C1CCN(CC2CCC(N3CCC(*)CC3)CC2)CC1]

In some embodiments, L is

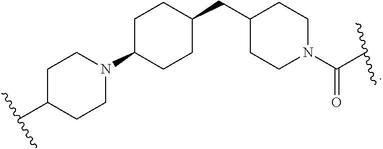 [SMILES: *C(=O)N1CCC(C[C@H]2CC[C@@H](N3CCC(*)CC3)CC2)CC1]

In In some embodiments, L is

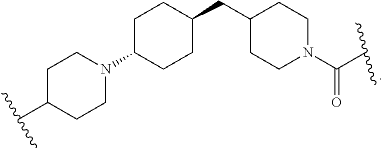 [SMILES: *C(=O)N1CCC(C[C@H]2CC[C@H](N3CCC(*)CC3)CC2)CC1]

In some embodiments, L is

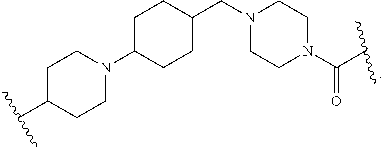 [SMILES: *C(=O)N1CCN(CC2CCC(N3CCC(*)CC3)CC2)CC1]

In some embodiments, L is

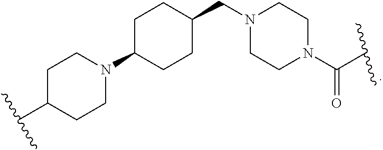 [SMILES: *C(=O)N1CCN(C[C@H]2CC[C@@H](N3CCC(*)CC3)CC2)CC1]

In In some embodiments, L is

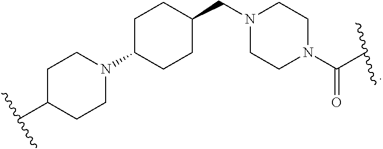 [SMILES: *C(=O)N1CCN(C[C@H]2CC[C@H](N3CCC(*)CC3)CC2)CC1]

In some embodiments, L is

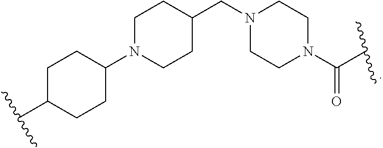 [SMILES: *C(=O)N1CCN(CC2CCN(C3CCC(*)CC3)CC2)CC1]

In some embodiments, L is

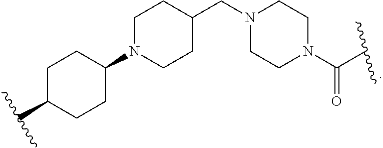 [SMILES: *C(=O)N1CCN(CC2CCN([C@H]3CC[C@@H](*)CC3)CC2)CC1]

In In some embodiments, L is

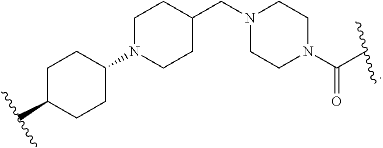 [SMILES: *C(=O)N1CCN(CC2CCN([C@H]3CC[C@H](*)CC3)CC2)CC1]

In some embodiments, L is

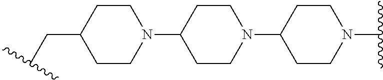 [SMILES: *CC1CCN(C2CCN(C3CCN(*)CC3)CC2)CC1]

In some embodiments, L is

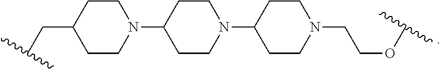 [SMILES: *CC1CCN(C2CCN(C3CCN(CCO*)CC3)CC2)CC1]

In some embodiments, L is

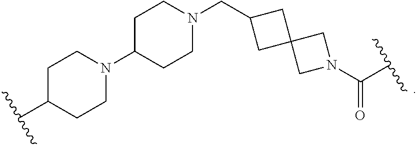 [SMILES: *C(=O)N1CC2(CC(CN3CCC(N4CCC(*)CC4)CC3)C2)C1]

In some embodiments, L is

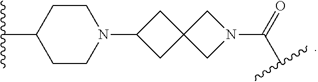 [SMILES: *C(=O)N1CC2(CC(N3CCC(*)CC3)C2)C1]

In some embodiments, L is

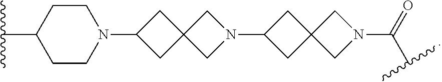 [SMILES: *C(=O)N1CC2(CC(N3CC4(CC(N5CCC(*)CC5)C4)C3)C2)C1]

In some embodiments, L is

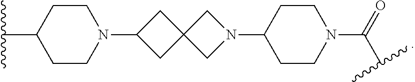 [SMILES: *C(=O)N1CCC(N2CC3(CC(N4CCC(*)CC4)C3)C2)CC1]

In some embodiments, L is

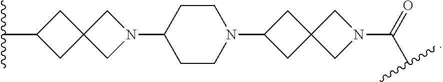 [SMILES: *C(=O)N1CC2(CC(N3CCC(N4CC5(CC(*)C5)C4)CC3)C2)C1]

In some embodiments, L is

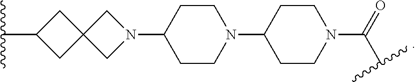 [SMILES: *C(=O)N1CCC(N2CCC(N3CC4(CC(*)C4)C3)CC2)CC1]

In some embodiments, L is

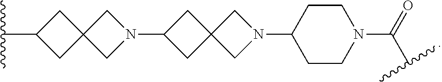 [SMILES: *C(=O)N1CCC(N2CC3(CC(N4CC5(CC(*)C5)C4)C3)C2)CC1]

In some embodiments, L is

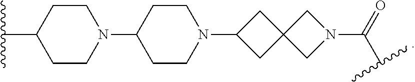 [SMILES: *C(=O)N1CC2(CC(N3CCC(N4CCC(*)CC4)CC3)C2)C1]

In some embodiments, L is

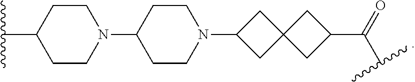 [SMILES: *C(=O)C1CC2(C1)CC(N1CCC(N3CCC(*)CC3)CC1)C2]

In some embodiments, L is

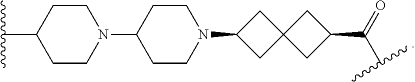 [SMILES: *C(=O)[C@H]1CC2(C1)C[C@H](N1CCC(N3CCC(*)CC3)CC1)C2]

In some embodiments, L is

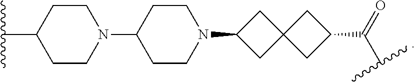 [SMILES: *C(=O)[C@H]1CC2(C1)C[C@H](N1CCC(N3CCC(*)CC3)CC1)C2]

In some embodiments, L is

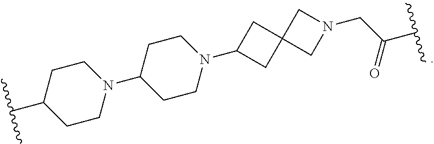 [SMILES: *C(=O)CN1CC2(CC(N3CCC(N4CCC(*)CC4)CC3)C2)C1]

In some embodiments, L is

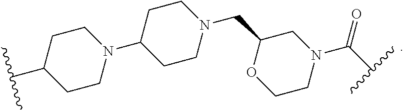 [SMILES: *C(=O)N1CCO[C@@H](CN2CCC(N3CCC(*)CC3)CC2)C1]

In some embodiments, L is

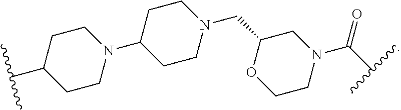 [SMILES: *C(=O)N1CCO[C@H](CN2CCC(N3CCC(*)CC3)CC2)C1]

In some embodiments, L is

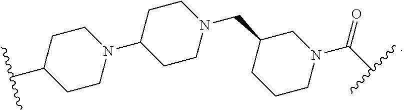 [SMILES: *C(=O)N1CCC[C@@H](CN2CCC(N3CCC(*)CC3)CC2)C1]

In some embodiments, L is

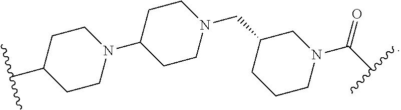 [SMILES: *C(=O)N1CCC[C@H](CN2CCC(N3CCC(*)CC3)CC2)C1]

In some embodiments, L is

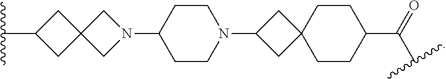 [SMILES: *C(=O)C1CCC2(CC1)CC(N1CCC(N3CC4(CC(*)C4)C3)CC1)C2]

In some embodiments, L is

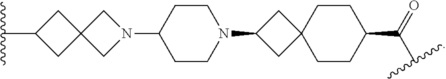 [SMILES: *C(=O)[C@H]1CCC2(CC1)C[C@H](N1CCC(N3CC4(CC(*)C4)C3)CC1)C2]

In some embodiments, L is

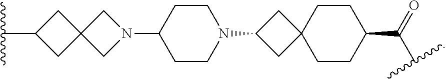 [SMILES: *C(=O)[C@H]1CCC2(CC1)C[C@H](N1CCC(N3CC4(CC(*)C4)C3)CC1)C2]

In some embodiments, L is

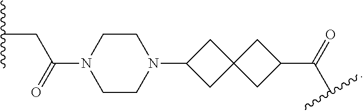 [SMILES: *CC(=O)N1CCN(C2CC3(CC(C(*)=O)C3)C2)CC1]

In some embodiments, L is

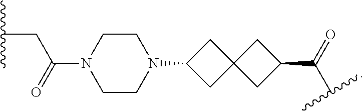 [SMILES: *CC(=O)N1CCN([C@H]2CC3(C[C@H](C(*)=O)C3)C2)CC1]

In some embodiments, L is

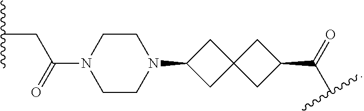 [SMILES: *CC(=O)N1CCN([C@H]2CC3(C[C@H](C(*)=O)C3)C2)CC1]

In some embodiments, L is

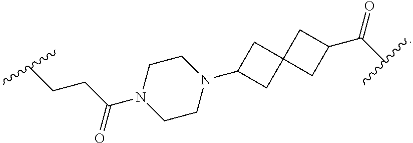 [SMILES: *CCC(=O)N1CCN(C2CC3(CC(C(*)=O)C3)C2)CC1]

In some embodiments, L is

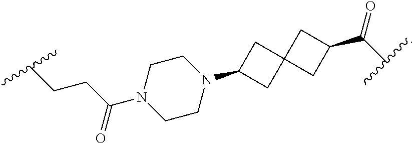 [SMILES: *CCC(=O)N1CCN([C@H]2CC3(C[C@H](C(*)=O)C3)C2)CC1]

In some embodiments, L is

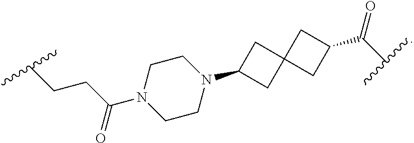 [SMILES: *CCC(=O)N1CCN([C@H]2CC3(C[C@H](C(*)=O)C3)C2)CC1]

In some embodiments, L is

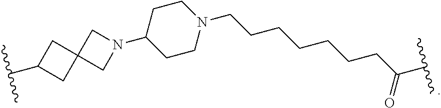 [SMILES: *C(=O)CCCCCCCN1CCC(N2CC3(CC(*)C3)C2)CC1]

In some embodiments, L is

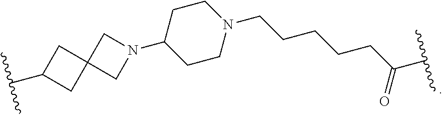 [SMILES: *C(=O)CCCCCN1CCC(N2CC3(CC(*)C3)C2)CC1]

In some embodiments, L is

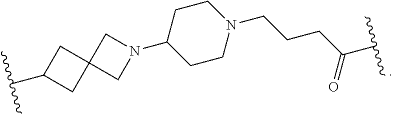 [SMILES: *C(=O)CCCN1CCC(N2CC3(CC(*)C3)C2)CC1]

In some embodiments, L is

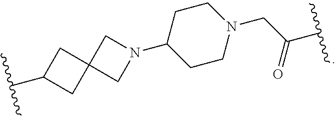 [SMILES: *C(=O)CN1CCC(N2CC3(CC(*)C3)C2)CC1]

In some embodiments, L is

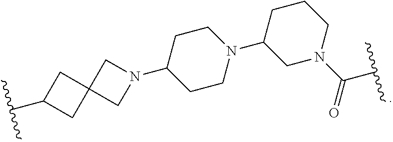 [SMILES: *C(=O)N1CCCC(N2CCC(N3CC4(CC(*)C4)C3)CC2)C1]

In some embodiments, L is

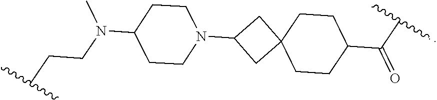 [SMILES: *CCN(C)C1CCN(C2CC3(CCC(C(*)=O)CC3)C2)CC1]

In some embodiments, L is

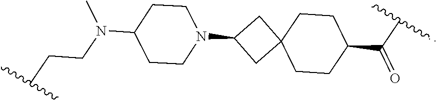 [SMILES: *CCN(C)C1CCN([C@H]2CC3(CC[C@H](C(*)=O)CC3)C2)CC1]

In some embodiments, L is

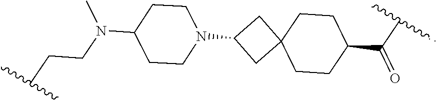 [SMILES: *CCN(C)C1CCN([C@H]2CC3(CC[C@H](C(*)=O)CC3)C2)CC1]

In some embodiments, L is selected from those depicted in Table 1, below.

Without limitation, the point of attachment of L to SMARCA and LBM can be, for example when L is

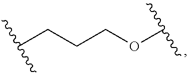 [SMILES: *CCCO*]

either

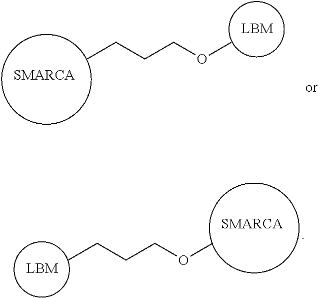

As defined above and described herein, in some embodiments, X is —C(O)—, —C(O)NR—, —SO₂—, —SO₂NR—, or an optionally substituted 5-membered heterocyclic ring.

In some embodiments, X is —C(O)—. In some embodiments, X is —C(O)NR—. In some embodiments, X is —SO₂—. In some embodiments, X is —SO₂NR—. In some embodiments, X is an optionally substituted 5-membered heterocyclic ring.

In some embodiments, X is —C(O)NH—. In some embodiments, X is

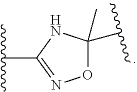 [SMILES: *C1=NOC(*)(C)N1]

In some embodiments, X is selected from those depicted in Table 1, below.

As defined above and described herein, in some embodiments, X¹ is a covalent bond or bivalent group selected from —O—, —C(O)—, —C(S)—, —C(R)₂—, —NR—, —S(O)—, or —SO₂—.

In some embodiments, X¹ is a covalent bond. In some embodiments, X¹ is —O—. In some embodiments, X¹ is —C(O)—. In some embodiments, X¹ is —C(S)—. In some embodiments, X¹ is —C(R)₂—. In some embodiments, X¹ is —NR—. In some embodiments, X¹ is —S(O)—. In some embodiments, X¹ is —SO₂—.

In some embodiments, X¹ is —CH₂—. In some embodiments, X¹ is

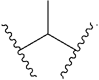 [SMILES: *C(*)C]

In some embodiments, X¹ is

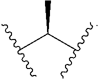

In some embodiments, X¹ is

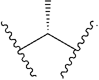

In some embodiments, X¹ is

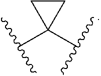 [SMILES: *C1(*)CC1]

In some embodiments, X¹ is

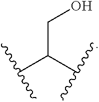 [SMILES: *C(*)CO]

In some embodiments, X¹ is

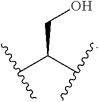

In some embodiments, X¹ is

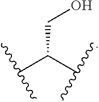

In some embodiments, X¹ is

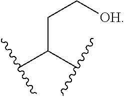 [SMILES: *C(*)CCO]

In some embodiments, X¹ is

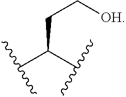

In some embodiments, X¹ is

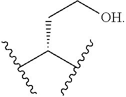

In some embodiments, X¹ is selected from those depicted in Table 1, below.

As defined above and described herein, in some embodiments, X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur.

In some embodiments, X² is an optionally substituted C₁₋₆ saturated or unsaturated alkylene. In some embodiments, X² is an optionally substituted phenylenyl. In some embodiments, X² is an optionally substituted 5-6 membered heteroarylenyl containing 1-4 heteroatoms independently selected from nitrogen, oxygen, and sulfur. In some embodiments, X² is an optionally substituted 4-11 membered saturated or partially unsaturated monocyclic, bicyclic, bridged bicyclic, or spirocyclic carbocyclylenyl. In some embodiments, X² is an optionally substituted 4-11 membered saturated or partially unsaturated monocyclic, bicyclic, bridged bicyclic, or spirocyclic heterocyclylenyl with 1-3 heteroatoms independently selected from nitrogen, oxygen, and sulfur.

In some embodiments, X² is

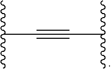 [SMILES: *C#C*]

In some embodiments, X² is

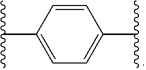 [SMILES: *c1ccc(*)cc1]

In some embodiments, X² is

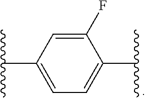 [SMILES: *c1ccc(*)c(F)c1]

In some embodiments, X² is

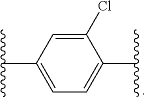 [SMILES: *c1ccc(*)c(Cl)c1]

In some embodiments, X² is

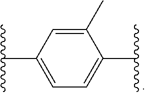 [SMILES: *c1ccc(*)c(C)c1]

In some embodiments, X² is

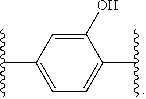 [SMILES: *c1ccc(*)c(O)c1]

In some embodiments, X² is

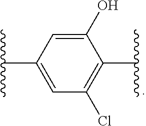 [SMILES: *c1cc(O)c(*)c(Cl)c1]

In some embodiments, X² is

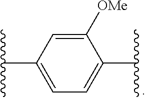 [SMILES: *c1ccc(*)c(OC)c1]

In some embodiments, X² is

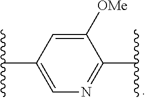 [SMILES: *c1cnc(*)c(OC)c1]

In some embodiments, X² is

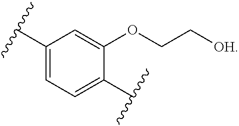 [SMILES: *c1ccc(*)c(OCCO)c1]

In some embodiments, X² is

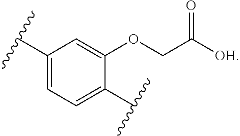 [SMILES: *c1ccc(*)c(OCC(=O)O)c1]

In some embodiments, X² is

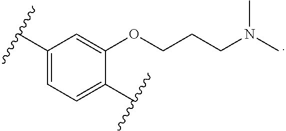 [SMILES: *c1ccc(*)c(OCCCN(C)C)c1]

In some embodiments, X² is

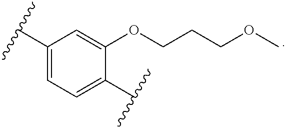 [SMILES: *c1ccc(*)c(OCCCOC)c1]

In some embodiments, X² is

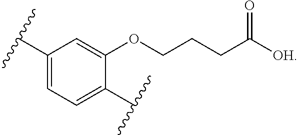 [SMILES: *c1ccc(*)c(OCCCC(=O)O)c1]

In some embodiments X² is

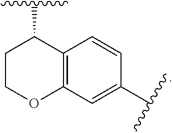 [SMILES: *c1ccc2c(c1)OCC[C@@H]2*]

In some embodiments, X² is

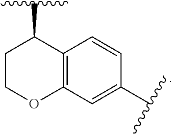 [SMILES: *c1ccc2c(c1)OCC[C@H]2*]

In some embodiments, X² is selected from those depicted in Table 1, below.

As defined above and described herein, in some embodiments, R¹ is R^{z}, —C(R)₂R^{z}, —OR, —SR, —N(R)₂, —C(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R.

In some embodiments, R¹ is R^{z}. In some embodiments, R¹ is —C(R)₂R^{z}. In some embodiments, R¹ is —OR. In some embodiments, R¹ is —SR. In some embodiments, R¹ is —N(R)₂. In some embodiments, R¹ is —C(R)₂OR. In some embodiments, R¹ is —C(R)₂N(R)₂. In some embodiments, R¹ is —C(R)₂NRC(O)R. In some embodiments, R¹ is —C(R)₂NRC(O)N(R)₂. In some embodiments, R¹ is —NRC(O)OR. In some embodiments, R¹ is —NRC(O)R. In some embodiments, R¹ is —NRC(O)N(R)₂. In some embodiments, R¹ is —NRSO₂R.

In some embodiments, R¹ is

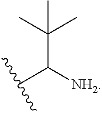 [SMILES: *C(N)C(C)(C)C]

In some embodiments, R¹ is

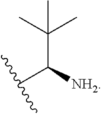 [SMILES: *[C@H](N)C(C)(C)C]

In some embodiments, R¹ is

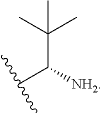 [SMILES: *[C@@H](N)C(C)(C)C]

In some embodiments, R¹ is

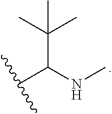 [SMILES: *C(NC)C(C)(C)C]

In some embodiments, R¹ is

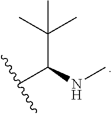 [SMILES: *[C@H](NC)C(C)(C)C]

In some embodiments, R¹ is

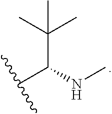 [SMILES: *[C@@H](NC)C(C)(C)C]

In some embodiments, R¹ is

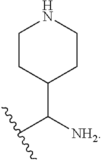 [SMILES: *C(N)C1CCNCC1]

In some embodiments, R¹ is

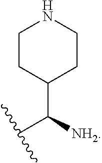 [SMILES: *[C@H](N)C1CCNCC1]

In some embodiments, R¹ is

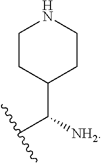 [SMILES: *[C@@H](N)C1CCNCC1]

In some embodiments, R¹ is

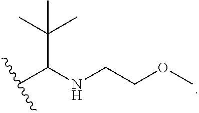 [SMILES: *C(NCCOC)C(C)(C)C]

In some embodiments, R¹ is

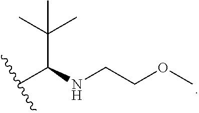 [SMILES: *[C@H](NCCOC)C(C)(C)C]

In some embodiments, R¹ is

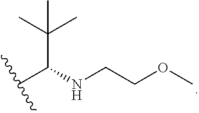 [SMILES: *[C@@H](NCCOC)C(C)(C)C]

In some embodiments, R¹ is

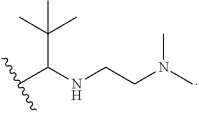 [SMILES: *C(NCCN(C)C)C(C)(C)C]

In some embodiments, R¹ is

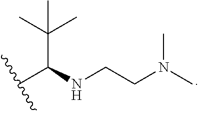 [SMILES: *[C@H](NCCN(C)C)C(C)(C)C]

In some embodiments, R¹ is

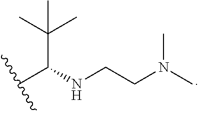 [SMILES: *[C@@H](NCCN(C)C)C(C)(C)C]

In some embodiments, R¹ is

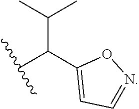 [SMILES: *C(c1ccno1)C(C)C]

In some embodiments, R¹ is

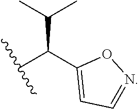 [SMILES: *[C@H](c1ccno1)C(C)C]

In some embodiments, R¹ is

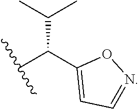 [SMILES: *[C@@H](c1ccno1)C(C)C]

In some embodiments, R¹ is

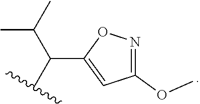 [SMILES: *C(c1cc(OC)no1)C(C)C]

In some embodiments, R¹ is

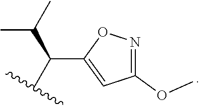 [SMILES: *[C@H](c1cc(OC)no1)C(C)C]

In some embodiments, R¹ is

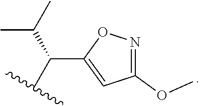 [SMILES: *[C@@H](c1cc(OC)no1)C(C)C]

In some embodiments, R¹ is

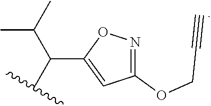 [SMILES: *C(c1cc(OCC#C)no1)C(C)C]

In some embodiments, R¹ is

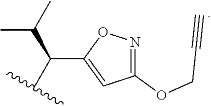 [SMILES: *[C@H](c1cc(OCC#C)no1)C(C)C]

In some embodiments, R¹ is

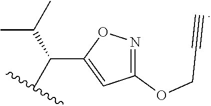 [SMILES: *[C@@H](c1cc(OCC#C)no1)C(C)C]

In some embodiments, R¹ is

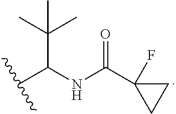 [SMILES: *C(NC(=O)C1(F)CC1)C(C)(C)C]

In some embodiments, R¹ is

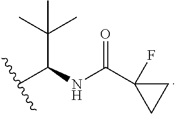 [SMILES: *[C@H](NC(=O)C1(F)CC1)C(C)(C)C]

In some embodiments, R¹ is

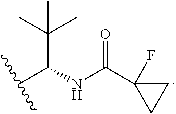 [SMILES: *[C@@H](NC(=O)C1(F)CC1)C(C)(C)C]

In some embodiments, R¹ is

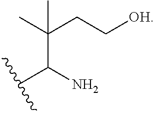 [SMILES: *C(N)C(C)(C)CCO]

In some embodiments, R¹ is

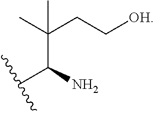 [SMILES: *[C@H](N)C(C)(C)CCO]

In some embodiments, R¹ is

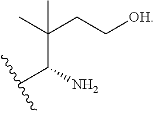 [SMILES: *[C@@H](N)C(C)(C)CCO]

In some embodiments, R¹ is

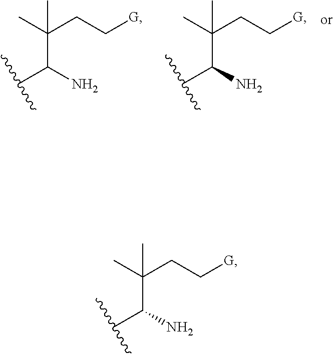

wherein G is —OH, —O(CH₂)₁₋₅CO₂R (e.g., —OCH₂CO₂H, etc.)), —OP(O)(OR)₂ (e.g., —OP(O)(OH)₂, etc.)), —O(CH₂)₁₋₅P(O)(OR)₂ (e.g., —O(CH₂)₂P(O)(OH)₂, etc.))

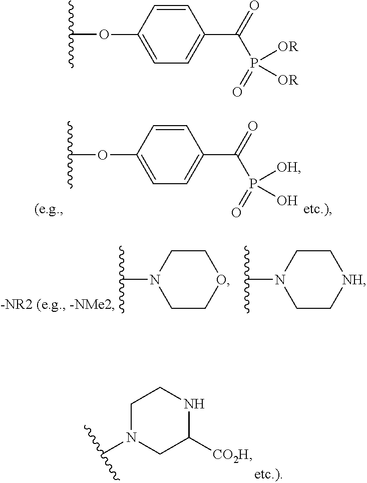

In some embodiments, G is —OH. In some embodiments, G is —OCH₂CO₂H. In some embodiments, G is

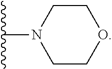 [SMILES: *N1CCOCC1]

In some embodiments, G is

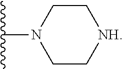 [SMILES: *N1CCNCC1]

In some embodiments, G is

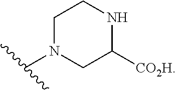 [SMILES: *N1CCNC(C(=O)O)C1]

In some embodiments, G is

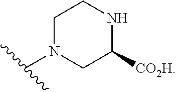 [SMILES: *N1CCN[C@@H](C(=O)O)C1]

In some embodiments, G is

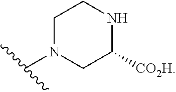 [SMILES: *N1CCN[C@H](C(=O)O)C1]

In some embodiments, R¹ is selected from those depicted in Table 1, below.

As defined above and described herein, in some embodiments, R² is hydrogen, halogen, —CN,

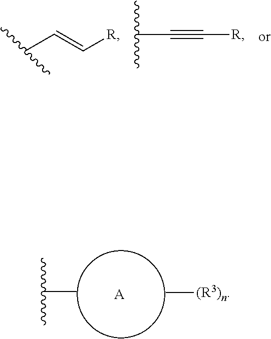

In some embodiments, R² is hydrogen. In some embodiments, R² is halogen. In some embodiments, R² is —CN. In some embodiments, R² is

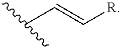

In some embodiments, R² is

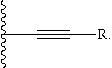

In some embodiments, R² is

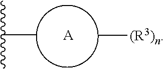

In some embodiments, R² is chloro. In some embodiments, R² is

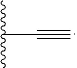 [SMILES: *C#C]

In some embodiments, R² is selected from those depicted in Table 1, below.

As defined above and described herein, in some embodiments, Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur.

In some embodiments, Ring A is phenyl. In some embodiments, Ring A is a 5-6 membered heteroaryl containing 1-4 heteroatoms independently selected from nitrogen, oxygen, and sulfur. In some embodiments, Ring A is 4 to 9-membered saturated or partially unsaturated monocyclic, bicyclic, bridged bicyclic, or spirocyclic carbocyclyl. In some embodiments, Ring A is a 4 to 9-membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring A is

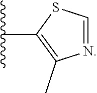 [SMILES: *c1scnc1C]

In some embodiments, Ring A is

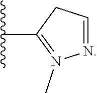

In some embodiments, Ring A is selected from those depicted in Table 1, below.

As defined above and described herein, in some embodiments, each of R³ is independently hydrogen, R^{z}, halogen, —CN, —NO₂, —OR, —SR, —N(R)₂, —Si(R)₃, —SO₂R, —SO₂NR₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R, or two R³ groups are optionally taken together to form an optionally substituted 5-7 membered partially unsaturated or aryl fused ring having 0-2 heteroatoms independently selected from nitrogen, oxygen, and sulfur.

In some embodiments, R³ is hydrogen. In some embodiments, R³ is R^{z}. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —N(R)₂. In some embodiments, R³ is —Si(R)₃. In some embodiments, R³ is —SO₂R. In some embodiments, R³ is —SO₂NR₂. In some embodiments, R³ is —S(O)R. In some embodiments, R³ is —C(O)R. In some embodiments, R³ is —C(O)OR. In some embodiments, R³ is —C(O)N(R)₂. In some embodiments, R³ is —C(O)N(R)OR. In some embodiments, R³ is —C(R)₂NRC(O)R. In some embodiments, R³ is —C(R)₂NRC(O)N(R)₂. In some embodiments, R³ is —OC(O)R. In some embodiments, R³ is —OC(O)N(R)₂. In some embodiments, R³ is —OP(O)(R)₂. In some embodiments, R³ is —OP(O)(OR)₂. In some embodiments, R³ is —OP(O)(OR)N(R)₂. In some embodiments, R³ is —OP(O)(N(R)₂)₂. In some embodiments, R³ is —N(R)C(O)OR. In some embodiments, R³ is —N(R)C(O)R. In some embodiments, R³ is —NRC(O)N(R)₂. In some embodiments, R³ is —N(R)SO₂R. In some embodiments, R³ is —NP(O)(R)₂. In some embodiments, R³ is —N(R)P(O)(OR)₂. In some embodiments, R³ is —N(R)P(O)(OR)N(R)₂. In some embodiments, R³ is —N(R)P(O)(N(R)₂)₂. In some embodiments, R³ is —N(R)SO₂R. In some embodiments, two R³ groups are optionally taken together to form an optionally substituted 5-7 membered partially unsaturated or aryl fused ring having 0-2 heteroatoms independently selected from nitrogen, oxygen, and sulfur.

In some embodiments, R³ is fluoro. In some embodiments, R³ is chloro. In some embodiments, R³ is methyl. In some embodiments, R³ is

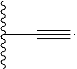 [SMILES: *C#C]

In some embodiments, R³ is

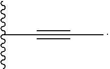 [SMILES: *C#CC]

In some embodiments, R³ is —OMe.

In some embodiments, R³ is selected from those depicted in Table 1, below.

As defined above and described herein, in some embodiments, n is 0, 1, 2, 4, or 5.

In some embodiments, n is 0. In some embodiments, n is 1. In some embodiments, n is 2. In some embodiments, n is 3. In some embodiments, n is 4. In some embodiments, n is 5.

In some embodiments, n is selected from those depicted in Table 1, below.

In some embodiments, SMARCA is

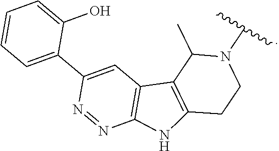 [SMILES: *N1CCc2[nH]c3nnc(-c4ccccc4O)cc3c2C1C]

In some embodiments, SMARCA is

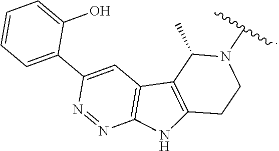 [SMILES: *N1CCc2[nH]c3nnc(-c4ccccc4O)cc3c2[C@@H]1C]

In some embodiments, SMARCA is

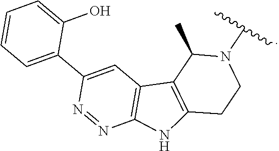 [SMILES: *N1CCc2[nH]c3nnc(-c4ccccc4O)cc3c2[C@H]1C]

In some embodiments, SMARCA is

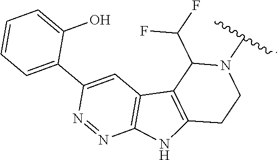 [SMILES: *N1CCc2[nH]c3nnc(-c4ccccc4O)cc3c2C1C(F)F]

In some embodiments, SMARCA is

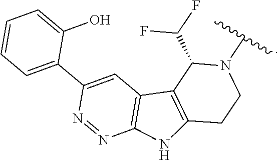 [SMILES: *N1CCc2[nH]c3nnc(-c4ccccc4O)cc3c2[C@@H]1C(F)F]

In some embodiments, SMARCA is

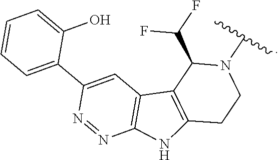 [SMILES: *N1CCc2[nH]c3nnc(-c4ccccc4O)cc3c2[C@H]1C(F)F]

In some embodiments, SMARCA is

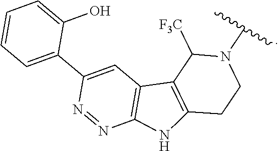 [SMILES: *N1CCc2[nH]c3nnc(-c4ccccc4O)cc3c2C1C(F)(F)F]

In some embodiments, SMARCA is

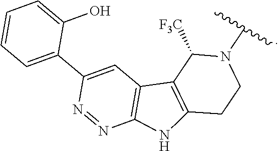 [SMILES: *N1CCc2[nH]c3nnc(-c4ccccc4O)cc3c2[C@@H]1C(F)(F)F]

In some embodiments, SMARCA is

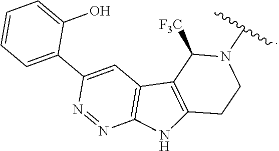 [SMILES: *N1CCc2[nH]c3nnc(-c4ccccc4O)cc3c2[C@H]1C(F)(F)F]

In some embodiments, SMARCA is

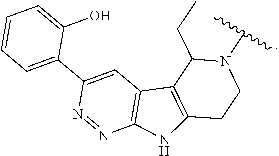 [SMILES: *N1CCc2[nH]c3nnc(-c4ccccc4O)cc3c2C1CC]

In some embodiments, SMARCA is

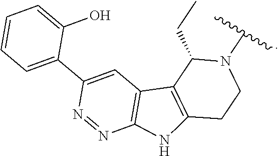 [SMILES: *N1CCc2[nH]c3nnc(-c4ccccc4O)cc3c2[C@@H]1CC]

In some embodiments, SMARCA is

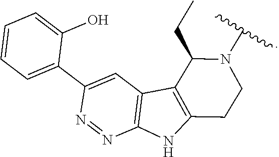 [SMILES: *N1CCc2[nH]c3nnc(-c4ccccc4O)cc3c2[C@H]1CC]

In some embodiments, SMARCA is

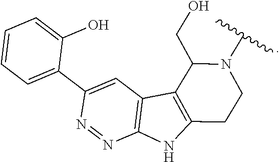 [SMILES: *N1CCc2[nH]c3nnc(-c4ccccc4O)cc3c2C1CO]

In some embodiments, SMARCA is

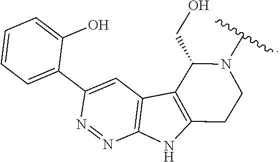 [SMILES: *N1CCc2[nH]c3nnc(-c4ccccc4O)cc3c2[C@@H]1CO]

In some embodiments, SMARCA is

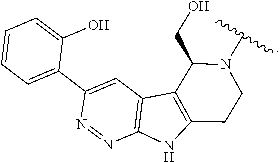 [SMILES: *N1CCc2[nH]c3nnc(-c4ccccc4O)cc3c2[C@H]1CO]

In some embodiments, SMARCA is

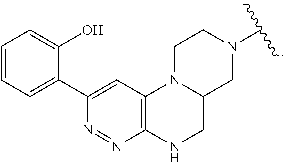 [SMILES: *N1CCN2c3cc(-c4ccccc4O)nnc3NCC2C1]

In some embodiments, SMARCA is

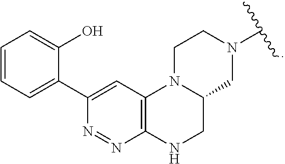 [SMILES: *N1CCN2c3cc(-c4ccccc4O)nnc3NC[C@H]2C1]

In some embodiments, SMARCA is

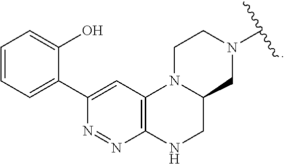 [SMILES: *N1CCN2c3cc(-c4ccccc4O)nnc3NC[C@@H]2C1]

In some embodiments, SMARCA is

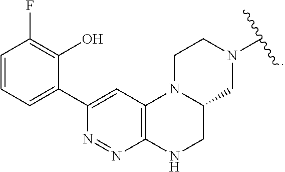 [SMILES: *N1CCN2c3cc(-c4cccc(F)c4O)nnc3NC[C@H]2C1]

In some embodiments, SMARCA is

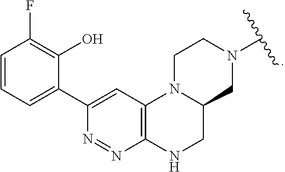 [SMILES: *N1CCN2c3cc(-c4cccc(F)c4O)nnc3NC[C@@H]2C1]

In some embodiments, SMARCA is

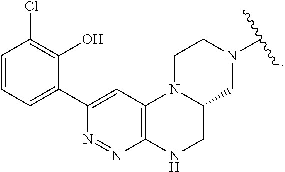 [SMILES: *N1CCN2c3cc(-c4cccc(Cl)c4O)nnc3NC[C@H]2C1]

In some embodiments, SMARCA is

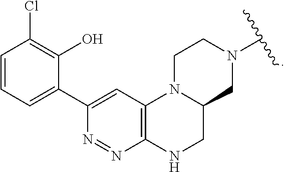 [SMILES: *N1CCN2c3cc(-c4cccc(Cl)c4O)nnc3NC[C@@H]2C1]

In some embodiments, SMARCA is

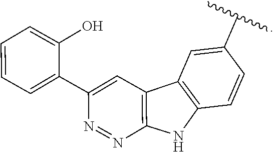 [SMILES: *c1ccc2[nH]c3nnc(-c4ccccc4O)cc3c2c1]

In some embodiments, SMARCA is selected from those depicted in Table 1, below.

In some embodiments, LBM is

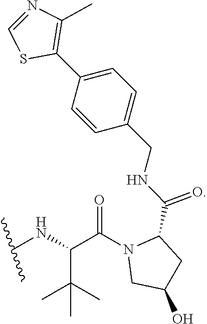 [SMILES: *N[C@H](C(=O)N1C[C@H](O)C[C@H]1C(=O)NCc1ccc(-c2scnc2C)cc1)C(C)(C)C]

In some embodiments, LBM is

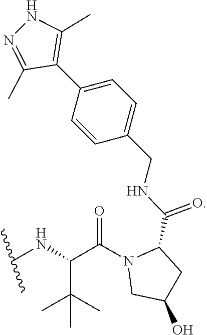 [SMILES: *N[C@H](C(=O)N1C[C@H](O)C[C@H]1C(=O)NCc1ccc(-c2c(C)n[nH]c2C)cc1)C(C)(C)C]

In some embodiments, LBM is

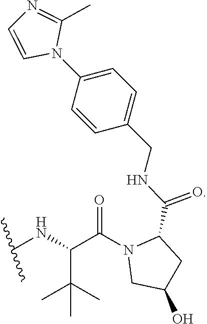 [SMILES: *N[C@H](C(=O)N1C[C@H](O)C[C@H]1C(=O)NCc1ccc(-n2ccnc2C)cc1)C(C)(C)C]

In some embodiments, LBM is

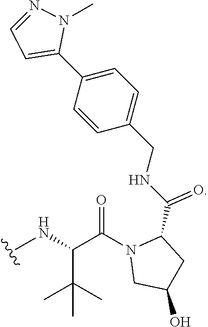

In some embodiments, LBM is

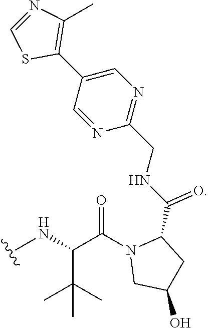

In some embodiments, LBM is

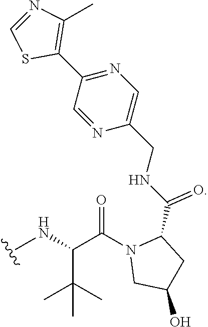

In some embodiments, LBM is

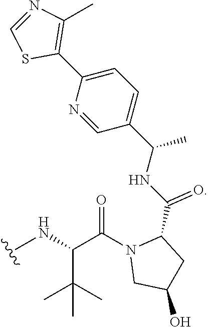

In some embodiments, LBM is

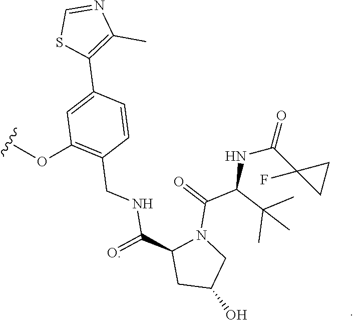

In some embodiments, LBM is

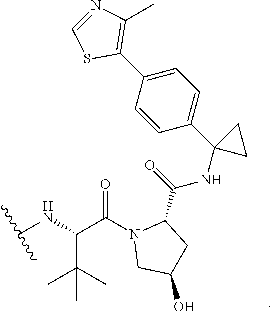 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

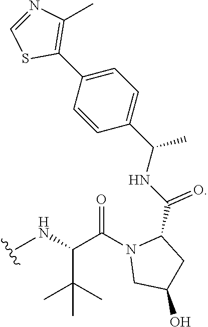

In some embodiments, LBM is

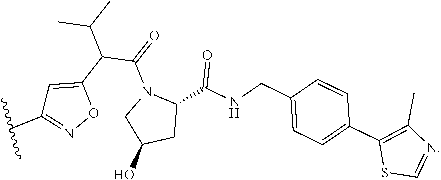 [SMILES: *c1cc(C(C(=O)N2C[C@H](O)C[C@H]2C(=O)NCc2ccc(-c3scnc3C)cc2)C(C)C)on1]

In some embodiments, LBM is

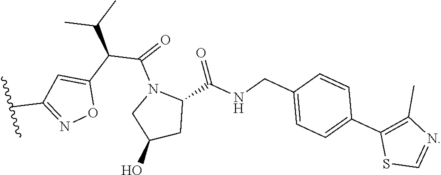 [SMILES: *c1cc([C@H](C(=O)N2C[C@H](O)C[C@H]2C(=O)NCc2ccc(-c3scnc3C)cc2)C(C)C)on1]

In some embodiments, LBM is

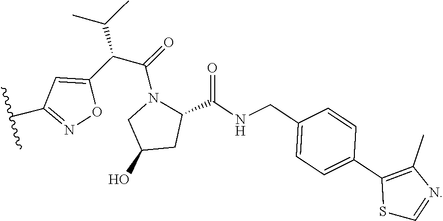 [SMILES: *c1cc([C@@H](C(=O)N2C[C@H](O)C[C@H]2C(=O)NCc2ccc(-c3scnc3C)cc2)C(C)C)on1]

In some embodiments, LBM is

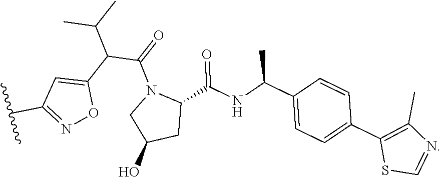 [SMILES: *c1cc(C(C(=O)N2C[C@H](O)C[C@H]2C(=O)N[C@@H](C)c2ccc(-c3scnc3C)cc2)C(C)C)on1]

In some embodiments, LBM is

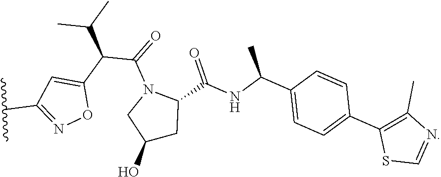 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

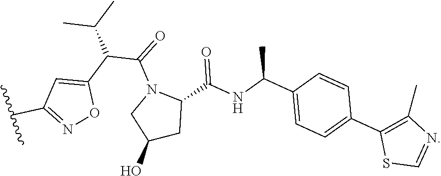

In some embodiments, LBM is

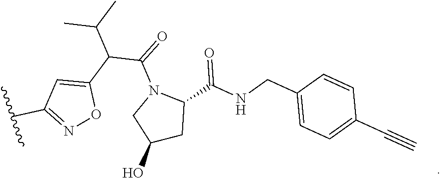 [SMILES: *c1cc(C(C(=O)N2C[C@H](O)C[C@H]2C(=O)NCc2ccc(C#C)cc2)C(C)C)on1]

In some embodiments, LBM is

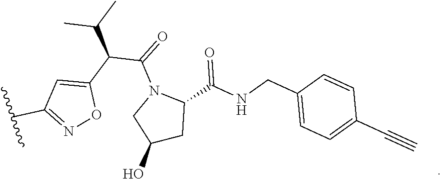 [SMILES: *c1cc([C@H](C(=O)N2C[C@H](O)C[C@H]2C(=O)NCc2ccc(C#C)cc2)C(C)C)on1]

In some embodiments, LBM is

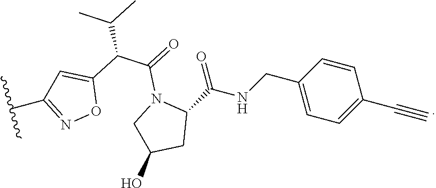 [SMILES: *c1cc([C@@H](C(=O)N2C[C@H](O)C[C@H]2C(=O)NCc2ccc(C#C)cc2)C(C)C)on1]

In some embodiments, LBM is

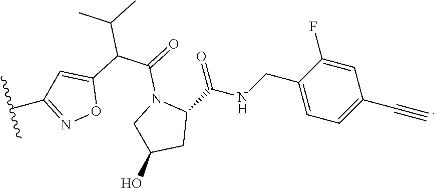 [SMILES: *c1cc(C(C(=O)N2C[C@H](O)C[C@H]2C(=O)NCc2ccc(C#C)cc2F)C(C)C)on1]

In some embodiments, LBM is

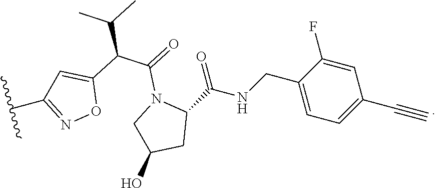 [SMILES: *c1cc([C@H](C(=O)N2C[C@H](O)C[C@H]2C(=O)NCc2ccc(C#C)cc2F)C(C)C)on1]

In some embodiments, LBM is

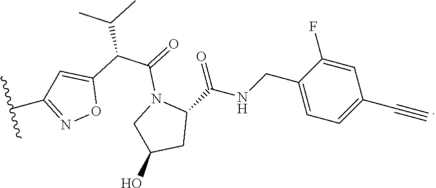 [SMILES: *c1cc([C@@H](C(=O)N2C[C@H](O)C[C@H]2C(=O)NCc2ccc(C#C)cc2F)C(C)C)on1]

In some embodiments, LBM is

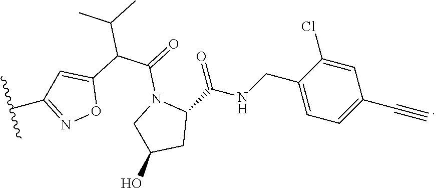 [SMILES: *c1cc(C(C(=O)N2C[C@H](O)C[C@H]2C(=O)NCc2ccc(C#C)cc2Cl)C(C)C)on1]

In some embodiments, LBM is

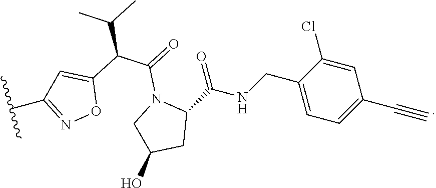 [SMILES: *c1cc([C@H](C(=O)N2C[C@H](O)C[C@H]2C(=O)NCc2ccc(C#C)cc2Cl)C(C)C)on1]

In some embodiments, LBM is

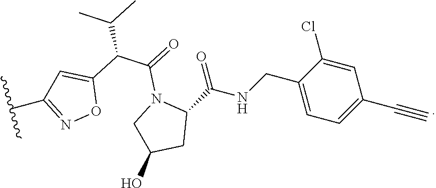 [SMILES: *c1cc([C@@H](C(=O)N2C[C@H](O)C[C@H]2C(=O)NCc2ccc(C#C)cc2Cl)C(C)C)on1]

In some embodiments, LBM is

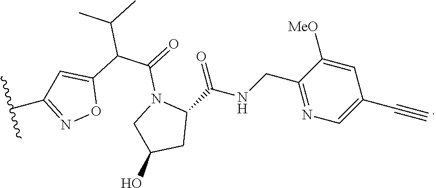 [SMILES: *c1cc(C(C(=O)N2C[C@H](O)C[C@H]2C(=O)NCc2ncc(C#C)cc2OC)C(C)C)on1]

In some embodiments, LBM is

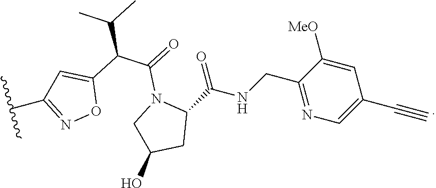 [SMILES: *c1cc([C@H](C(=O)N2C[C@H](O)C[C@H]2C(=O)NCc2ncc(C#C)cc2OC)C(C)C)on1]

In some embodiments, LBM is

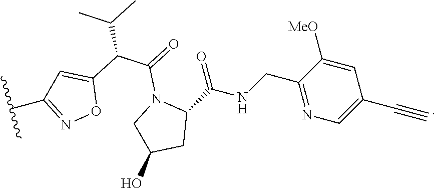 [SMILES: *c1cc([C@@H](C(=O)N2C[C@H](O)C[C@H]2C(=O)NCc2ncc(C#C)cc2OC)C(C)C)on1]

In some embodiments, LBM is

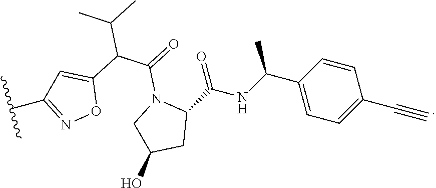 [SMILES: *c1cc(C(C(=O)N2C[C@H](O)C[C@H]2C(=O)N[C@@H](C)c2ccc(C#C)cc2)C(C)C)on1]

In some embodiments, LBM is

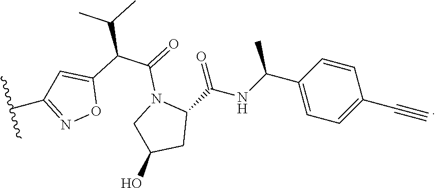 [SMILES: *c1cc([C@H](C(=O)N2C[C@H](O)C[C@H]2C(=O)N[C@@H](C)c2ccc(C#C)cc2)C(C)C)on1]

In some embodiments, LBM is

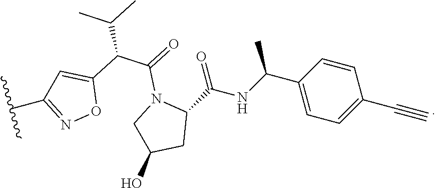 [SMILES: *c1cc([C@@H](C(=O)N2C[C@H](O)C[C@H]2C(=O)N[C@@H](C)c2ccc(C#C)cc2)C(C)C)on1]

In some embodiments, LBM is

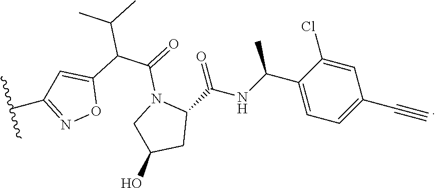 [SMILES: *c1cc(C(C(=O)N2C[C@H](O)C[C@H]2C(=O)N[C@@H](C)c2ccc(C#C)cc2Cl)C(C)C)on1]

In some embodiments, LBM is

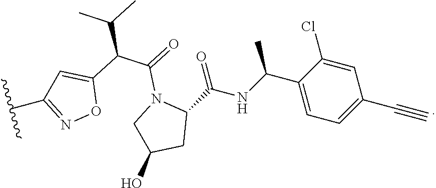 [SMILES: *c1cc([C@H](C(=O)N2C[C@H](O)C[C@H]2C(=O)N[C@@H](C)c2ccc(C#C)cc2Cl)C(C)C)on1]

In some embodiments, LBM is

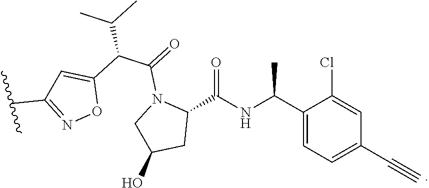 [SMILES: *c1cc([C@@H](C(=O)N2C[C@H](O)C[C@H]2C(=O)N[C@@H](C)c2ccc(C#C)cc2Cl)C(C)C)on1]

In some embodiments, LBM is

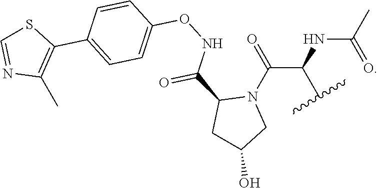 [SMILES: *[C@H](NC(C)=O)C(=O)N1C[C@H](O)C[C@H]1C(=O)NOc1ccc(-c2scnc2C)cc1]

In some embodiments, LBM is

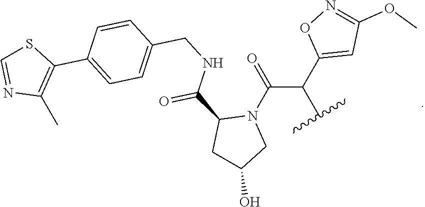 [SMILES: *C(C(=O)N1C[C@H](O)C[C@H]1C(=O)NCc1ccc(-c2scnc2C)cc1)c1cc(OC)no1]

In some embodiments, LBM is

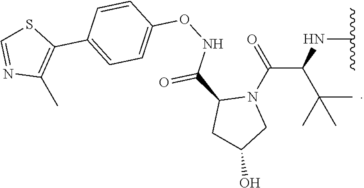 [SMILES: *N[C@H](C(=O)N1C[C@H](O)C[C@H]1C(=O)NOc1ccc(-c2scnc2C)cc1)C(C)(C)C]

In some embodiments, LBM is

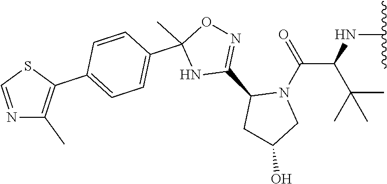 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

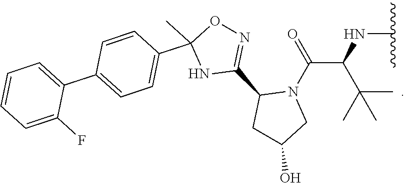 [SMILES: *N[C@H](C(=O)N1C[C@H](O)C[C@H]1C1=NOC(C)(c2ccc(-c3ccccc3F)cc2)N1)C(C)(C)C]

In some embodiments, LBM is

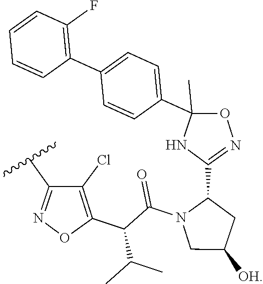 [SMILES: *c1noc([C@H](C(=O)N2C[C@H](O)C[C@H]2C2=NOC(C)(c3ccc(-c4ccccc4F)cc3)N2)C(C)C)c1Cl]

In some embodiments, LBM is

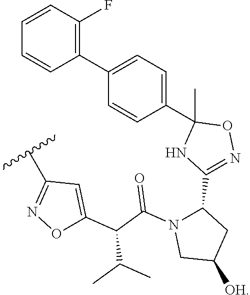 [SMILES: *c1cc([C@H](C(=O)N2C[C@H](O)C[C@H]2C2=NOC(C)(c3ccc(-c4ccccc4F)cc3)N2)C(C)C)on1]

In some embodiments, LBM is

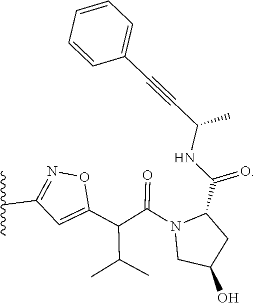 [SMILES: *c1cc(C(C(=O)N2C[C@H](O)C[C@H]2C(=O)N[C@@H](C)C#Cc2ccccc2)C(C)C)on1]

In some embodiments, LBM is

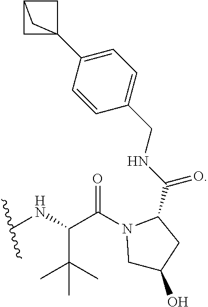 [SMILES: *N[C@H](C(=O)N1C[C@H](O)C[C@H]1C(=O)NCc1ccc(C23CC(C2)C3)cc1)C(C)(C)C]

In some embodiments, LBM is

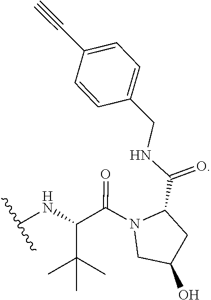 [SMILES: *N[C@H](C(=O)N1C[C@H](O)C[C@H]1C(=O)NCc1ccc(C#C)cc1)C(C)(C)C]

In some embodiments, LBM is

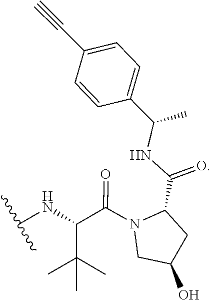 [SMILES: *N[C@H](C(=O)N1C[C@H](O)C[C@H]1C(=O)N[C@@H](C)c1ccc(C#C)cc1)C(C)(C)C]

In some embodiments, LBM is

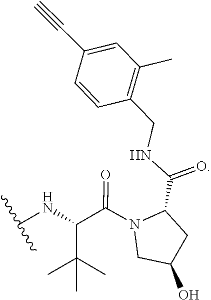 [SMILES: *N[C@H](C(=O)N1C[C@H](O)C[C@H]1C(=O)NCc1ccc(C#C)cc1C)C(C)(C)C]

In some embodiments, LBM is

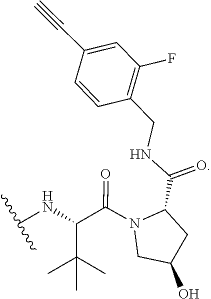 [SMILES: *N[C@H](C(=O)N1C[C@H](O)C[C@H]1C(=O)NCc1ccc(C#C)cc1F)C(C)(C)C]

In some embodiments, LBM is

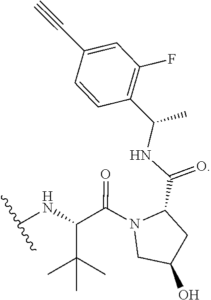 [SMILES: *N[C@H](C(=O)N1C[C@H](O)C[C@H]1C(=O)N[C@@H](C)c1ccc(C#C)cc1F)C(C)(C)C]

In some embodiments, LBM is

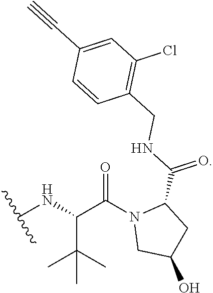 [SMILES: *N[C@H](C(=O)N1C[C@H](O)C[C@H]1C(=O)NCc1ccc(C#C)cc1Cl)C(C)(C)C]

In some embodiments, LBM is

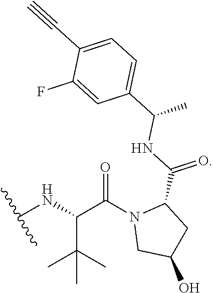 [SMILES: *N[C@H](C(=O)N1C[C@H](O)C[C@H]1C(=O)N[C@@H](C)c1ccc(C#C)c(F)c1)C(C)(C)C]

In some embodiments, LBM is

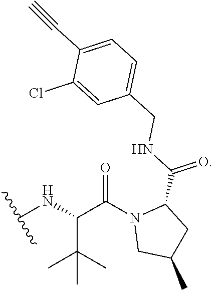 [SMILES: *N[C@H](C(=O)N1C[C@H](C)C[C@H]1C(=O)NCc1ccc(C#C)c(Cl)c1)C(C)(C)C]

In some embodiments, LBM is

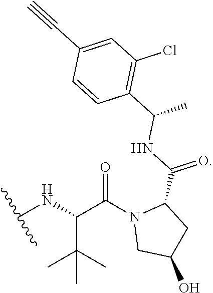 [SMILES: *N[C@H](C(=O)N1C[C@H](O)C[C@H]1C(=O)N[C@@H](C)c1ccc(C#C)cc1Cl)C(C)(C)C]

In some embodiments, LBM is

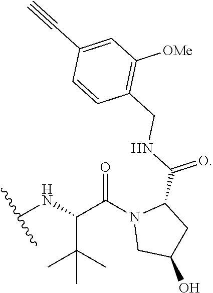 [SMILES: *N[C@H](C(=O)N1C[C@H](O)C[C@H]1C(=O)NCc1ccc(C#C)cc1OC)C(C)(C)C]

In some embodiments, LBM is

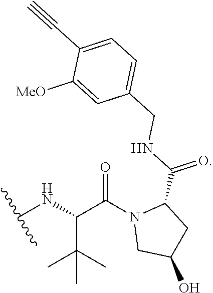 [SMILES: *N[C@H](C(=O)N1C[C@H](O)C[C@H]1C(=O)NCc1ccc(C#C)c(OC)c1)C(C)(C)C]

In some embodiments, LBM is

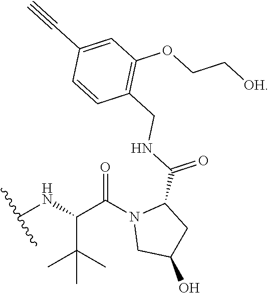 [SMILES: *N[C@H](C(=O)N1C[C@H](O)C[C@H]1C(=O)NCc1ccc(C#C)cc1OCCO)C(C)(C)C]

In some embodiments, LBM is

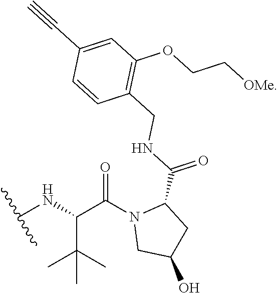 [SMILES: *N[C@H](C(=O)N1C[C@H](O)C[C@H]1C(=O)NCc1ccc(C#C)cc1OCCOC)C(C)(C)C]

In some embodiments, LBM is

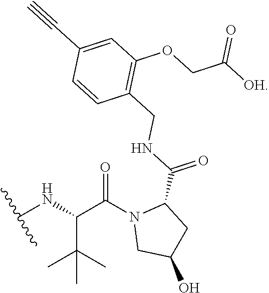 [SMILES: *N[C@H](C(=O)N1C[C@H](O)C[C@H]1C(=O)NCc1ccc(C#C)cc1OCC(=O)O)C(C)(C)C]

In some embodiments, LBM is

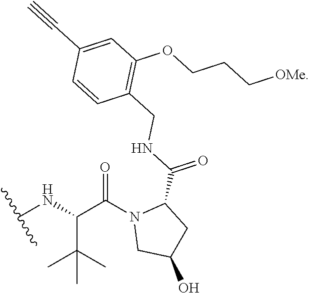 [SMILES: *N[C@H](C(=O)N1C[C@H](O)C[C@H]1C(=O)NCc1ccc(C#C)cc1OCCCOC)C(C)(C)C]

In some embodiments, LBM is

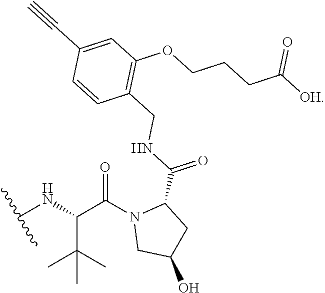 [SMILES: *N[C@H](C(=O)N1C[C@H](O)C[C@H]1C(=O)NCc1ccc(C#C)cc1OCCCC(=O)O)C(C)(C)C]

In some embodiments, LBM is

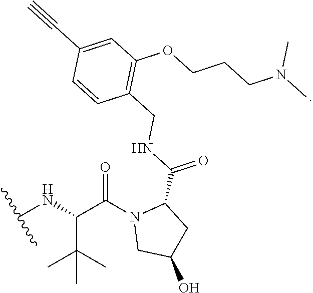 [SMILES: *N[C@H](C(=O)N1C[C@H](O)C[C@H]1C(=O)NCc1ccc(C#C)cc1OCCCN(C)C)C(C)(C)C]

In some embodiments, LBM is

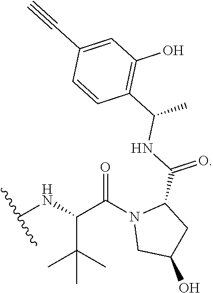 [SMILES: *N[C@H](C(=O)N1C[C@H](O)C[C@H]1C(=O)N[C@@H](C)c1ccc(C#C)cc1O)C(C)(C)C]

In some embodiments, LBM is

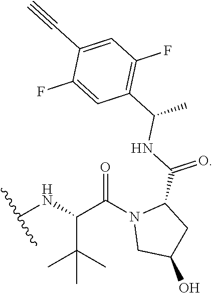 [SMILES: *N[C@H](C(=O)N1C[C@H](O)C[C@H]1C(=O)N[C@@H](C)c1cc(F)c(C#C)cc1F)C(C)(C)C]

In some embodiments, LBM is

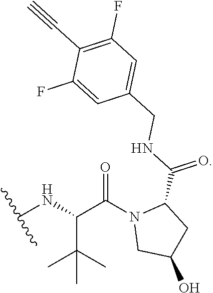 [SMILES: *N[C@H](C(=O)N1C[C@H](O)C[C@H]1C(=O)NCc1cc(F)c(C#C)c(F)c1)C(C)(C)C]

In some embodiments, LBM is

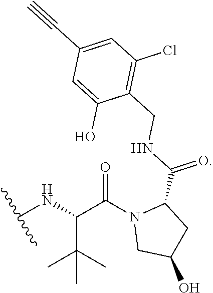 [SMILES: *N[C@H](C(=O)N1C[C@H](O)C[C@H]1C(=O)NCc1c(O)cc(C#C)cc1Cl)C(C)(C)C]

In some embodiments, LBM is

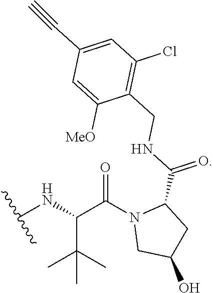 [SMILES: *N[C@H](C(=O)N1C[C@H](O)C[C@H]1C(=O)NCc1c(Cl)cc(C#C)cc1OC)C(C)(C)C]

In some embodiments, LBM is

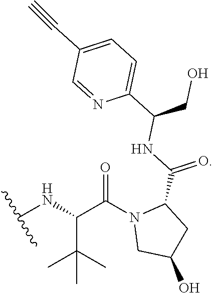 [SMILES: *N[C@H](C(=O)N1C[C@H](O)C[C@H]1C(=O)N[C@H](CO)c1ccc(C#C)cn1)C(C)(C)C]

In some embodiments, LBM is

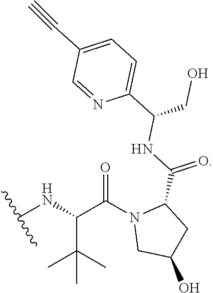 [SMILES: *N[C@H](C(=O)N1C[C@H](O)C[C@H]1C(=O)N[C@@H](CO)c1ccc(C#C)cn1)C(C)(C)C]

In some embodiments, LBM is

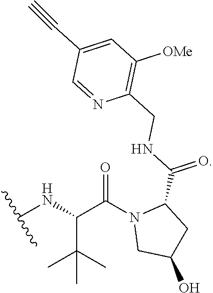 [SMILES: *N[C@H](C(=O)N1C[C@H](O)C[C@H]1C(=O)NCc1ncc(C#C)cc1OC)C(C)(C)C]

In some embodiments, LBM is

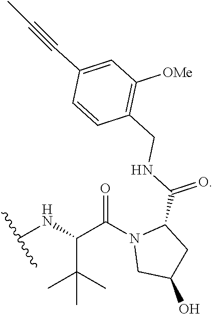 [SMILES: *N[C@H](C(=O)N1C[C@H](O)C[C@H]1C(=O)NCc1ccc(C#CC)cc1OC)C(C)(C)C]

In some embodiments, LBM is

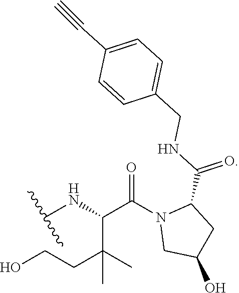 [SMILES: *N[C@H](C(=O)N1C[C@H](O)C[C@H]1C(=O)NCc1ccc(C#C)cc1)C(C)(C)CCO]

In some embodiments, LBM is

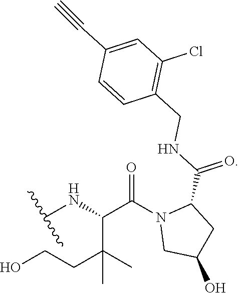 [SMILES: *N[C@H](C(=O)N1C[C@H](O)C[C@H]1C(=O)NCc1ccc(C#C)cc1Cl)C(C)(C)CCO]

In some embodiments, LBM is

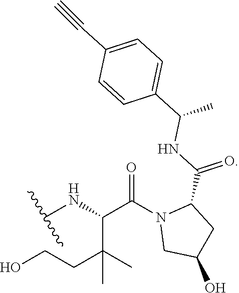 [SMILES: *N[C@H](C(=O)N1C[C@H](O)C[C@H]1C(=O)N[C@@H](C)c1ccc(C#C)cc1)C(C)(C)CCO]

In some embodiments, LBM is

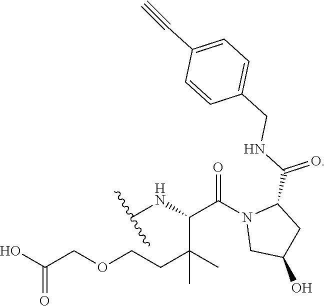 [SMILES: *N[C@H](C(=O)N1C[C@H](O)C[C@H]1C(=O)NCc1ccc(C#C)cc1)C(C)(C)CCOCC(=O)O]

In some embodiments, LBM is

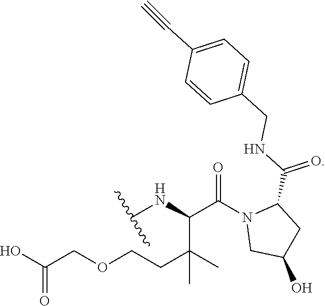 [SMILES: *N[C@@H](C(=O)N1C[C@H](O)C[C@H]1C(=O)NCc1ccc(C#C)cc1)C(C)(C)CCOCC(=O)O]

In some embodiments, LBM is

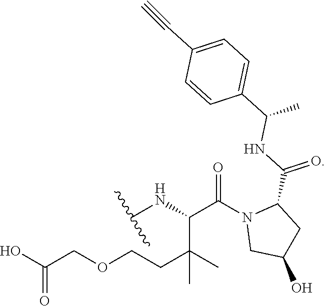 [SMILES: *N[C@H](C(=O)N1C[C@H](O)C[C@H]1C(=O)N[C@@H](C)c1ccc(C#C)cc1)C(C)(C)CCOCC(=O)O]

In some embodiments, LBM is

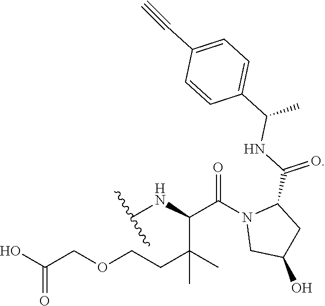 [SMILES: *N[C@@H](C(=O)N1C[C@H](O)C[C@H]1C(=O)N[C@@H](C)c1ccc(C#C)cc1)C(C)(C)CCOCC(=O)O]

In some embodiments, LBM is

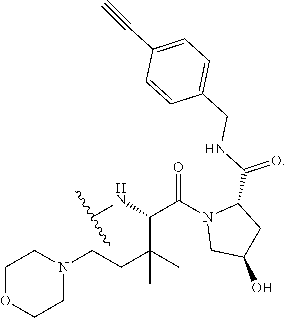 [SMILES: *N[C@H](C(=O)N1C[C@H](O)C[C@H]1C(=O)NCc1ccc(C#C)cc1)C(C)(C)CCN1CCOCC1]

In some embodiments, LBM is

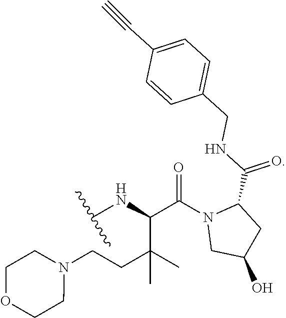 [SMILES: *N[C@@H](C(=O)N1C[C@H](O)C[C@H]1C(=O)NCc1ccc(C#C)cc1)C(C)(C)CCN1CCOCC1]

In some embodiments, LBM is

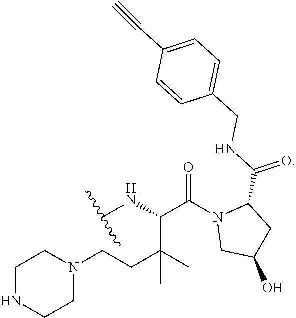 [SMILES: *N[C@H](C(=O)N1C[C@H](O)C[C@H]1C(=O)NCc1ccc(C#C)cc1)C(C)(C)CCN1CCNCC1]

In some embodiments, LBM is

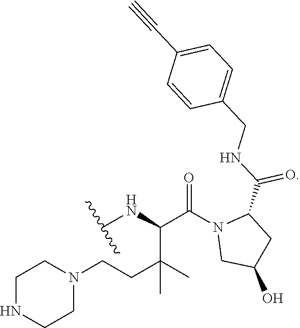 [SMILES: *N[C@@H](C(=O)N1C[C@H](O)C[C@H]1C(=O)NCc1ccc(C#C)cc1)C(C)(C)CCN1CCNCC1]

In some embodiments, LBM is

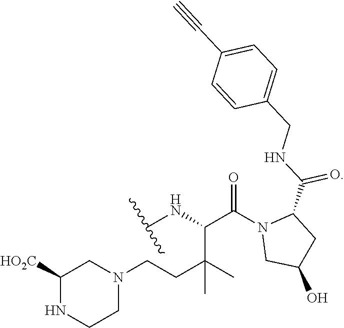 [SMILES: *N[C@H](C(=O)N1C[C@H](O)C[C@H]1C(=O)NCc1ccc(C#C)cc1)C(C)(C)CCN1CCN[C@@H](C(=O)O)C1]

In some embodiments, LBM is

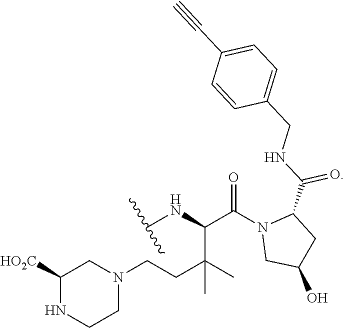 [SMILES: *N[C@@H](C(=O)N1C[C@H](O)C[C@H]1C(=O)NCc1ccc(C#C)cc1)C(C)(C)CCN1CCN[C@@H](C(=O)O)C1]

In some embodiments, LBM is

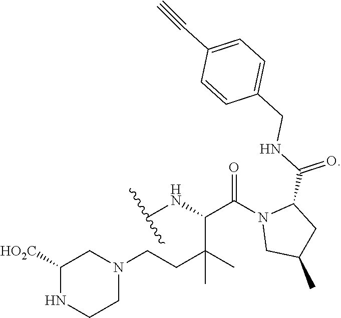 [SMILES: *N[C@H](C(=O)N1C[C@H](C)C[C@H]1C(=O)NCc1ccc(C#C)cc1)C(C)(C)CCN1CCN[C@H](C(=O)O)C1]

In some embodiments, LBM is

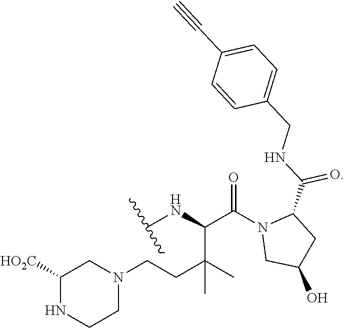 [SMILES: *N[C@@H](C(=O)N1C[C@H](O)C[C@H]1C(=O)NCc1ccc(C#C)cc1)C(C)(C)CCN1CCN[C@H](C(=O)O)C1]

In some embodiments, LBM is selected from those depicted in Table 1, below.

In some embodiments, the present invention provides a compound having the structures of SMARCA, LBM, and L presented above.

In certain embodiments, the present invention provides a compound of formula I-a, wherein R^{w} is

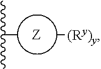

Ring Z is phenyl, one R^{y} is —OH, L^{x} is a covalent bond, X is —C(O)NH—, and X² is phenylenyl as shown, to provide a compound of formula I-a-1:

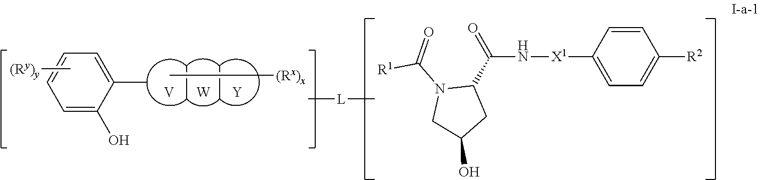

or a pharmaceutically acceptable salt thereof, wherein each of L, R^{x}, R^{y}, Ring V, Ring W, Ring Y, x, y, R¹, R², and X¹ is as defined above and described in embodiments herein, both singly and in combination.

In certain embodiments, the present invention provides a compound of formula I-a, wherein R^{w} is

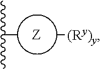

Ring Z is phenyl, one R^{y} is —OH, L is a covalent bond, Ring V is

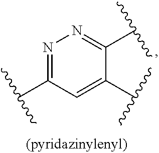 [SMILES: *c1cc(*)c(*)nn1]

X is —C(O)NH—, X² is phenylenyl, R² is

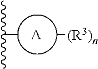

as shown, to provide a compound of formula I-a-2:

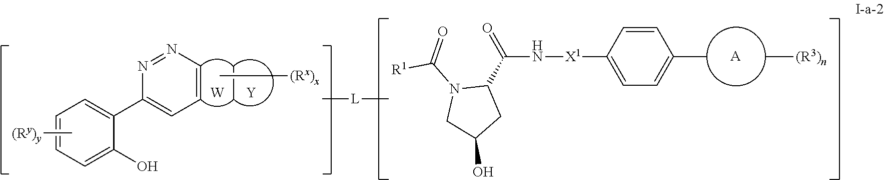

or a pharmaceutically acceptable salt thereof, wherein each of L, R^{x}, R^{y}, Ring W, Ring Y, x, y, Ring A, R¹, R³, n, and X¹ is as defined above and described in embodiments herein, both singly and in combination.

In certain embodiments, the present invention provides a compound of formula I-a, wherein R^{w} is

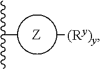

Ring Z is phenyl, one R^{y} is —OH, L^{x} is a covalent bond, Ring V is

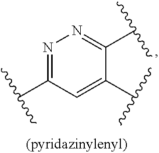 [SMILES: *c1cc(*)c(*)nn1]

### Ring W is

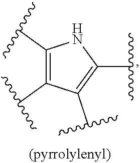 [SMILES: *c1[nH]c(*)c(*)c1*]

X is —C(O)NH—, and X² is phenylenyl as shown, to provide a compound of formula I-a-3:

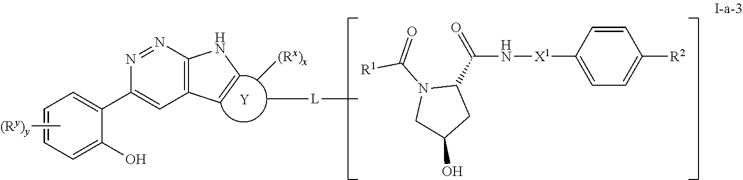

or a pharmaceutically acceptable salt thereof, wherein each of L, R^{x}, R^{y}, Ring V, Ring W, Ring Y, x, y, R¹, R², and X¹ is as defined above and described in embodiments herein, both singly and in combination.

In certain embodiments, the present invention provides a compound of formula I-a, wherein R^{w} is

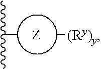

Ring Z is phenyl, one R^{y} is —OH, L^{x} is a covalent bond, Ring V is

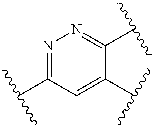 [SMILES: *c1cc(*)c(*)nn1]

### Ring W is

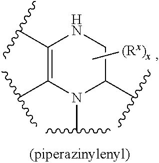

X is —C(O)NH—, X² is phenylenyl as shown, to provide a compound of formula I-a-4:

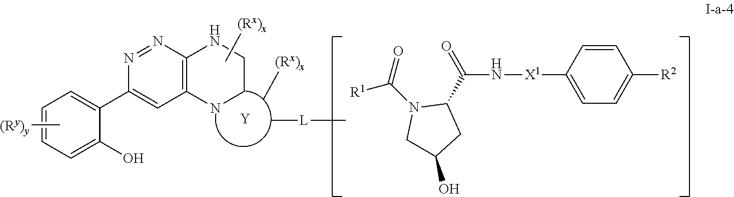

or a pharmaceutically acceptable salt thereof, wherein each of L, R^{x}, R^{y}, Ring V, Ring W, Ring Y, x, y, R¹, R², and X¹ is as defined above and described in embodiments herein, both singly and in combination.

In certain embodiments, the present invention provides a compound of formula I-a, wherein R^{w} is

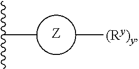

Ring Z is phenyl, one R^{y} is —OH, L^{x} is a covalent bond, Ring V is

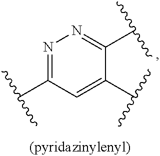 [SMILES: *c1cc(*)c(*)nn1]

### R is

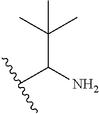 [SMILES: *C(N)C(C)(C)C]

(where one of the H groups of the NH₂ group is replaced with -L-), X is —C(O)NH—, X² is phenylenyl, R² is

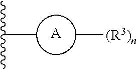

as shown, to provide a compound of formula I-a-5:

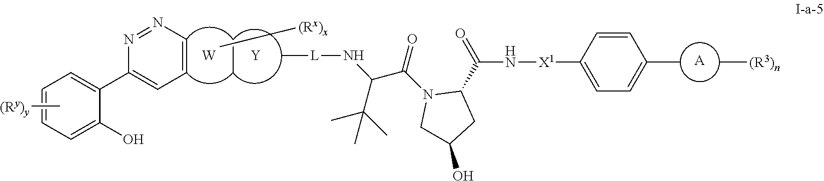

or a pharmaceutically acceptable salt thereof, wherein each of L, R^{x}, R^{y}, Ring W, Ring Y, x, y, Ring A, R³, n, and X is as defined above and described in embodiments herein, both singly and in combination.

In certain embodiments, the present invention provides a compound of formula I-a, wherein R^{w} is

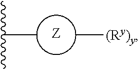

Ring Z is phenyl, one R^{y} is —OH, L^{x} is a covalent bond, Ring V is pyridazinylenyl, R¹ is

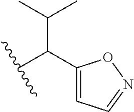 [SMILES: *C(c1ccno1)C(C)C]

(where one of the H groups of the isoxazolyl group is replaced with -L-), X is —C(O)NH—, X² is phenylenyl, R² is

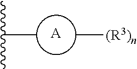

as shown, to provide a compound of formula I-a-6:

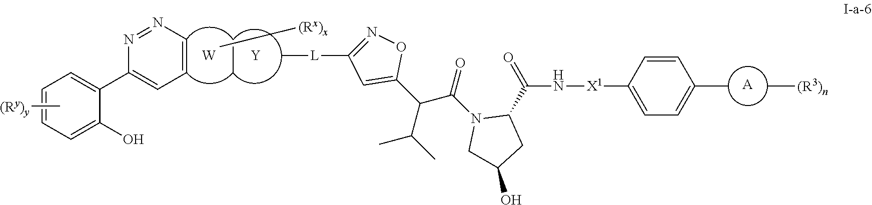

or a pharmaceutically acceptable salt thereof, wherein each of L, R^{x}, R^{y}, Ring W, Ring Y, x, y, Ring A, R³, n, and X¹ is as defined above and described in embodiments herein, both singly and in combination.

In certain embodiments, the present invention provides a compound of formula I-a, wherein R^{w} is

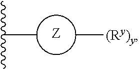

Ring Z is phenyl, one R^{y} is —OH, L^{x} is a covalent bond, Ring V is

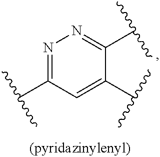 [SMILES: *c1cc(*)c(*)nn1]

### R is

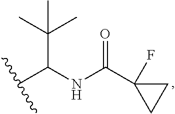 [SMILES: *C(NC(=O)C1(F)CC1)C(C)(C)C]

X is —C(O)NH—, X² is phenylenyl, R² is

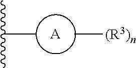

as shown, to provide a compound of formula I-a-7:

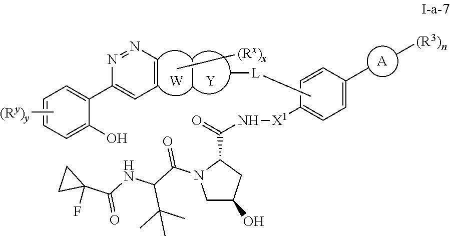

or a pharmaceutically acceptable salt thereof, wherein each of L, R^{x}, R^{y}, Ring W, Ring Y, x, y, Ring A, R³, n, and X¹ is as defined above and described in embodiments herein, both singly and in combination.

In certain embodiments, the present invention provides a compound of formula I-a, wherein R^{w} is

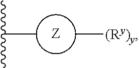

Ring Z is phenyl, one R^{y} is —OH, L^{x} is a covalent bond, X is —C(O)NH—, and R² is

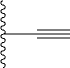 [SMILES: *C#C]

as shown, to provide a compound of formula I-a-8:

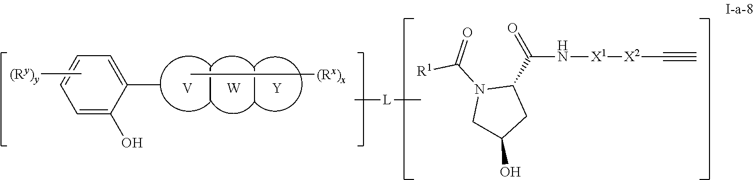

or a pharmaceutically acceptable salt thereof, wherein each of L, R^{x}, R^{y}, Ring V, Ring W, Ring Y, x, y, R¹, X¹, and X² is as defined above and described in embodiments herein, both singly and in combination.

In certain embodiments, the present invention provides a compound of formula I-a, wherein R^{w} is

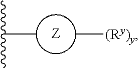

Ring Z is phenyl, one R^{y} is —OH, L^{x} is a covalent bond, Ring V is

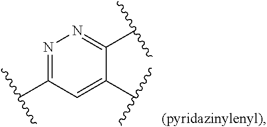 [SMILES: *c1cc(*)c(*)nn1]

X is —C(O)NH—, X² is phenylenyl, and R² is

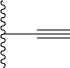 [SMILES: *C#C]

as shown, to provide a compound of formula I-a-9:

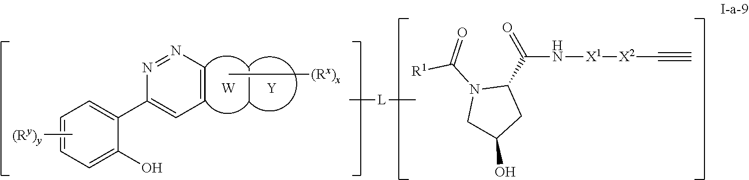

or a pharmaceutically acceptable salt thereof, wherein each of L, R^{x}, R^{y}, Ring W, Ring Y, x, y, R¹, R³, X¹, and X² is as defined above and described in embodiments herein, both singly and in combination.

In certain embodiments, the present invention provides a compound of formula I-a, wherein R^{w} is

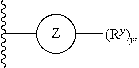

Ring Z is phenyl, one R^{y} is —OH, L^{x} is a covalent bond, Ring V is

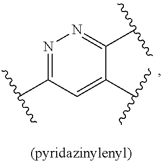 [SMILES: *c1cc(*)c(*)nn1]

### Ring W is

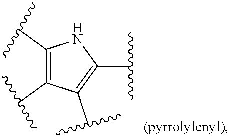 [SMILES: *c1[nH]c(*)c(*)c1*]

X is —C(O)NH—, X² is phenylenyl, and R² is

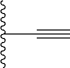 [SMILES: *C#C]

as shown, to provide a compound of formula I-a-10:

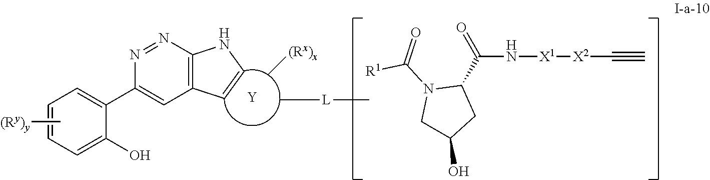

or a pharmaceutically acceptable salt thereof, wherein each of L, R^{x}, R^{y}, Ring Y, x, y, R¹, X¹, and X² is as defined above and described in embodiments herein, both singly and in combination.

In certain embodiments, the present invention provides a compound of formula I-a, wherein R^{w} is

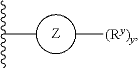

Ring Z is phenyl, one R^{y} is —OH, L^{x} is a covalent bond, Ring V is

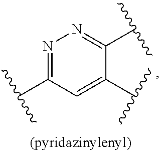 [SMILES: *c1cc(*)c(*)nn1]

### R¹ is

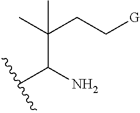

(where one of the H groups of the NH₂ group is replaced with -L-), X is —C(O)NH—, and R² is

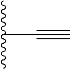 [SMILES: *C#C]

as shown, to provide a compound of formula I-a-11:

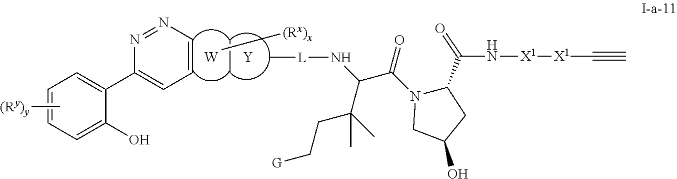

or a pharmaceutically acceptable salt thereof, wherein each of L, R^{x}, R^{y}, Ring W, Ring Y, x, y, R², G, X¹, and X² is as defined above and described in embodiments herein, both singly and in combination.

In certain embodiments, the present invention provides a compound of formula I-a, wherein R^{w} is

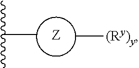

Ring Z is phenyl, one R^{y} is —OH, L^{x} is a covalent bond, Ring V is pyridazinylenyl, R¹ is

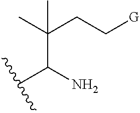

(where one of the H groups of the NH₂ group is replaced with -L-), X is —C(O)NH—, X² is phenylenyl, and R² is

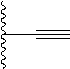 [SMILES: *C#C]

as shown, to provide a compound of formula I-a-12:

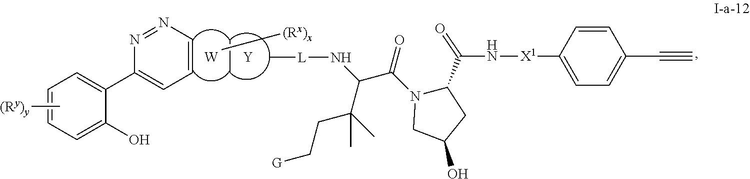

or a pharmaceutically acceptable salt thereof, wherein each of L, R^{x}, R^{y}, Ring W, Ring Y, x, y, G, and X¹ is as defined above and described in embodiments herein, both singly and in combination.

In certain embodiments, the present invention provides a compound of formula I-a, wherein R^{w} is

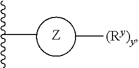

Ring Z is phenyl, one R^{y} is —OH, L^{x} is a covalent bond, Ring V is

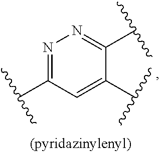 [SMILES: *c1cc(*)c(*)nn1]

### Ring W is

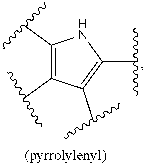 [SMILES: *c1[nH]c(*)c(*)c1*]

X is —C(O)NH—, X² is phenylenyl, and R² is

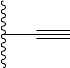 [SMILES: *C#C]

as shown, to provide a compound of formula I-a-13:

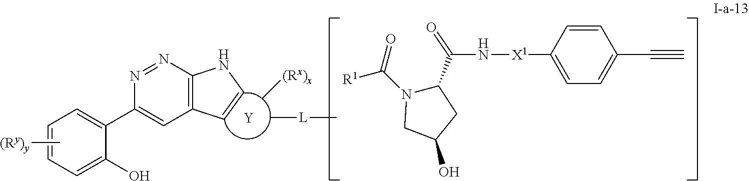

or a pharmaceutically acceptable salt thereof, wherein each of L, R^{x}, R^{y}, Ring Y, x, y, R¹, and X¹ is as defined above and described in embodiments herein, both singly and in combination.

In certain embodiments, the present invention provides a compound of formula I-a, wherein R^{w} is

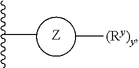

Ring Z is phenyl, one R^{y} is —OH, L^{x} is a covalent bond, Ring V is

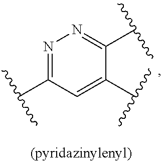 [SMILES: *c1cc(*)c(*)nn1]

### Ring W is

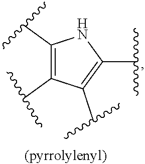 [SMILES: *c1[nH]c(*)c(*)c1*]

### R¹ is

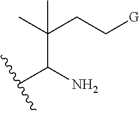

(where one of the H groups of the NH₂ group is replaced with -L-), X is —C(O)NH—, X² is phenylenyl, and R² is

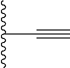 [SMILES: *C#C]

as shown, to provide a compound of formula I-a-14:

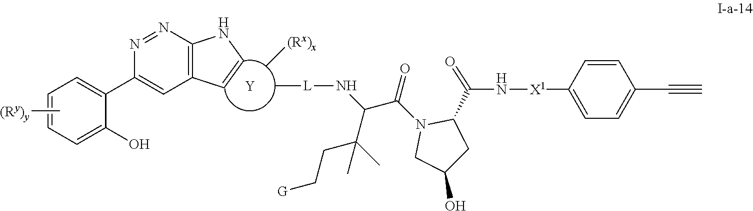

or a pharmaceutically acceptable salt thereof, wherein each of L, R^{x}, R^{y}, Ring Y, x, y, G, and X is as defined above and described in embodiments herein, both singly and in combination.

In certain embodiments, the present invention provides a compound of formula I-a, wherein R^{w} is

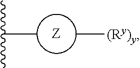

Ring Z is phenyl, one R^{y} is —OH, L^{x} is a covalent bond, Ring V is

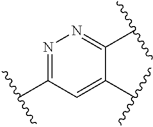 [SMILES: *c1cc(*)c(*)nn1]

### R¹ is

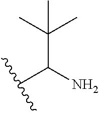 [SMILES: *C(N)C(C)(C)C]

(where one of the H groups of the NH₂ group is replaced with -L-), X is —C(O)NH—, R² is

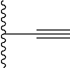 [SMILES: *C#C]

as shown, to provide a compound of formula I-a-15:

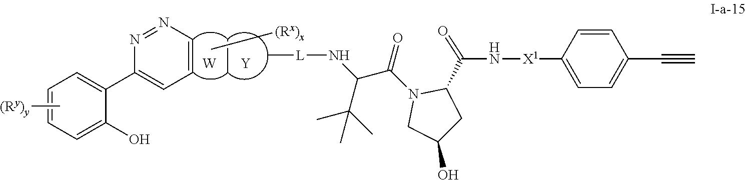

or a pharmaceutically acceptable salt thereof, wherein each of L, R^{x}, R^{y}, Ring W, Ring Y, x, y, X¹, and X² is as defined above and described in embodiments herein, both singly and in combination.

In certain embodiments, the present invention provides a compound of formula I-a, wherein R^{w} is

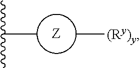

Ring Z is phenyl, one R^{y} is —OH, L^{x} is a covalent bond, Ring V is

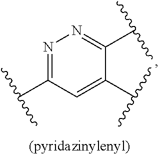 [SMILES: *c1cc(*)c(*)nn1]

### Ring W is

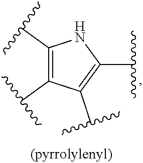 [SMILES: *c1[nH]c(*)c(*)c1*]

### R¹ is

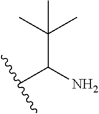 [SMILES: *C(N)C(C)(C)C]

(where one of the H groups of the NH₂ group is replaced with -L-), X is —C(O)NH—, and R² is

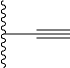 [SMILES: *C#C]

as shown, to provide a compound of formula I-a-16:

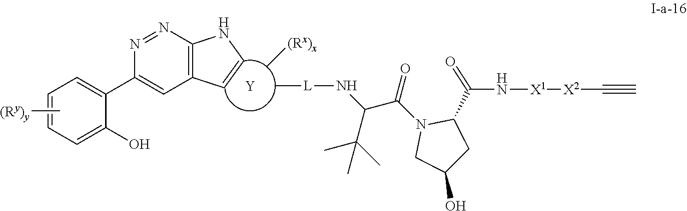

or a pharmaceutically acceptable salt thereof, wherein each of L, R^{x}, R^{y}, Ring Y, x, y, X¹, and X² is as defined above and described in embodiments herein, both singly and in combination.

In certain embodiments, the present invention provides a compound of formula I-a, wherein R^{w} is

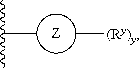

Ring Z is phenyl, one R^{y} is —OH, L^{x} is a covalent bond, Ring V is

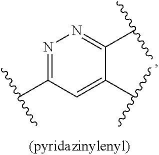 [SMILES: *c1cc(*)c(*)nn1]

### R¹ is

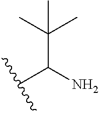 [SMILES: *C(N)C(C)(C)C]

(where one of the H groups of the NH₂ group is replaced with -L-), L is —Cy-Cy-Cy-CO—, X is —C(O)NH—, R² is

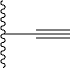 [SMILES: *C#C]

as shown, to provide a compound of formula I-a-17:

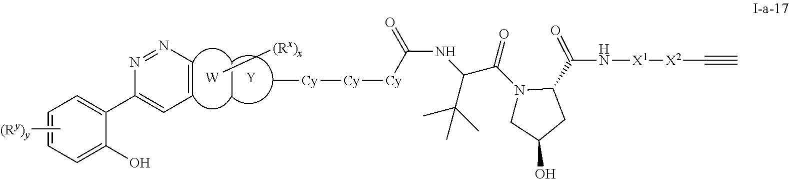

or a pharmaceutically acceptable salt thereof, wherein each of R^{x}, R^{y}, Ring W, Ring Y, x, y, X¹, X² and each -Cy- is as defined above and described in embodiments herein, both singly and in combination.

In certain embodiments, the present invention provides a compound of formula I-a, wherein R^{w} is

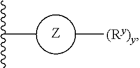

Ring Z is phenyl, one R^{y} is —OH, L^{x} is a covalent bond, Ring V is

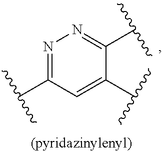 [SMILES: *c1cc(*)c(*)nn1]

### Ring W is

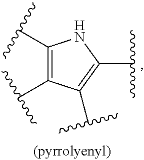 [SMILES: *c1[nH]c(*)c(*)c1*]

### R¹ is

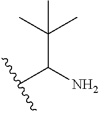 [SMILES: *C(N)C(C)(C)C]

(where one of the H groups of the NH₂ group is replaced with -L-), L is -Cy-Cy-Cy-CO—, X is —C(O)NH— and R² is

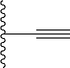 [SMILES: *C#C]

as shown, to provide a compound of formula I-a-18:

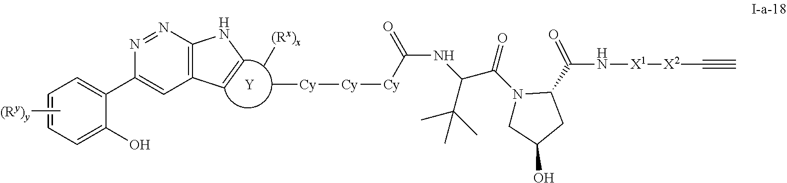

or a pharmaceutically acceptable salt thereof, wherein each of R^{x}, R^{y}, Ring Y, x, y, X¹, X² and each -Cy- is as defined above and described in embodiments herein, both singly and in combination.

In certain embodiments, the present invention provides a compound of formula I-a, wherein R^{w} is

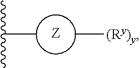

Ring Z is phenyl, one R^{y} is —OH, L^{x} is a covalent bond, Ring V is

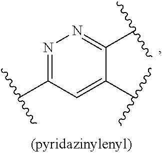 [SMILES: *c1cc(*)c(*)nn1]

### R¹ is

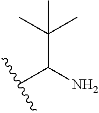 [SMILES: *C(N)C(C)(C)C]

(where one of the H groups of the NH₂ group is replaced with -L-), L is

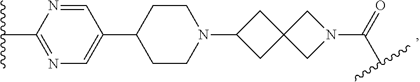 [SMILES: *C(=O)N1CC2(CC(N3CCC(c4cnc(*)nc4)CC3)C2)C1]

### X is —C(O)NH—, R² is

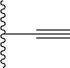 [SMILES: *C#C]

as shown, to provide a compound of formula I-a-19:

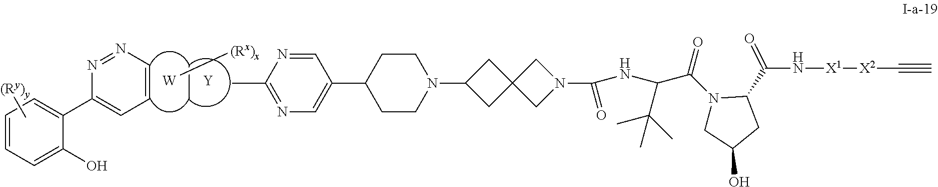

or a pharmaceutically acceptable salt thereof, wherein each of R^{x}, R^{y}, Ring W, Ring Y, x, y, X¹, X² and each -Cy- is as defined above and described in embodiments herein, both singly and in combination.

In certain embodiments, the present invention provides a compound of formula I-a, wherein R^{w} is

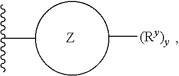

Ring Z is phenyl, one R^{y} is —OH, L^{x} is a covalent bond, Ring V is

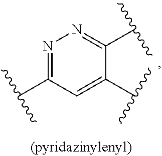 [SMILES: *c1cc(*)c(*)nn1]

### Ring W is

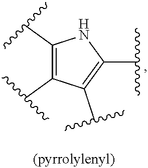 [SMILES: *c1[nH]c(*)c(*)c1*]

### R¹ is

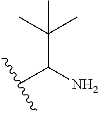 [SMILES: *C(N)C(C)(C)C]

(where one of the H groups of the NH₂ group is replaced with -L-), L is

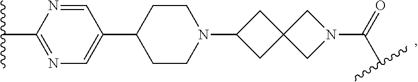 [SMILES: *C(=O)N1CC2(CC(N3CCC(c4cnc(*)nc4)CC3)C2)C1]

### X is —C(O)NH—, and R² is

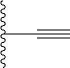 [SMILES: *C#C]

as shown, to provide a compound of formula I-a-20.

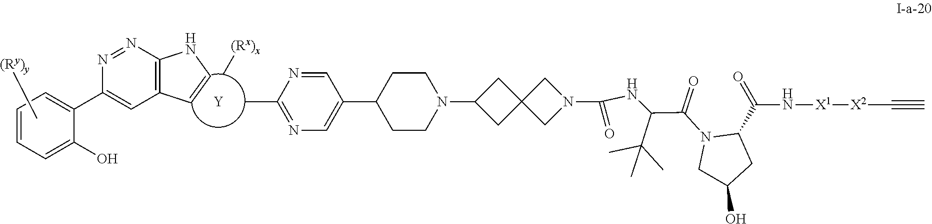

or a pharmaceutically acceptable salt thereof, wherein each of R^{x}, R^{y}, Ring Y, x, y, X¹, and X² is as defined above and described in embodiments herein, both singly and in combination.

Exemplary compounds of the invention are set forth in Table 1, below.

TABLE 1
Exemplary Compounds

| I-# | Structure |
|---|---|
| I-1 | 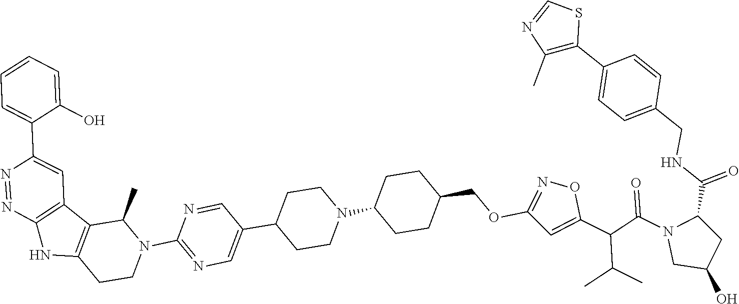 [SMILES: Cc1ncsc1-c1ccc(CNC(=O)[C@@H]2C[C@@H](O)CN2C(=O)C(c2cc(OC[C@H]3CC[C@H](N4CCC(c5cnc(N6CCc7[nH]c8nnc(-c9ccccc9O)cc8c7[C@H]6C)nc5)CC4)CC3)no2)C(C)C)cc1] |
| I-2 | 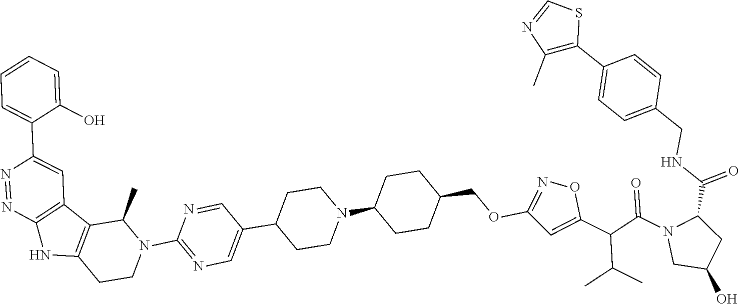 [SMILES: Cc1ncsc1-c1ccc(CNC(=O)[C@@H]2C[C@@H](O)CN2C(=O)C(c2cc(OC[C@H]3CC[C@@H](N4CCC(c5cnc(N6CCc7[nH]c8nnc(-c9ccccc9O)cc8c7[C@H]6C)nc5)CC4)CC3)no2)C(C)C)cc1] |
| I-3 | 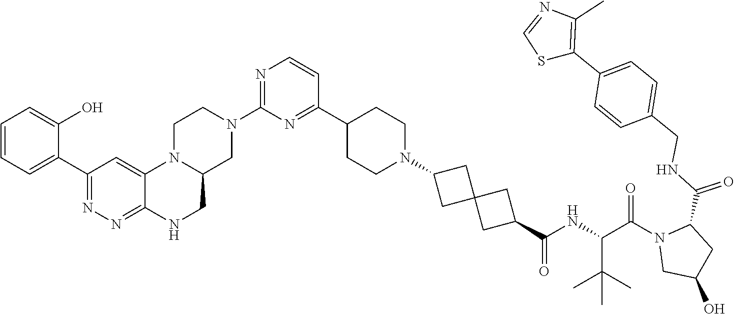 [SMILES: Cc1ncsc1-c1ccc(CNC(=O)[C@@H]2C[C@@H](O)CN2C(=O)[C@@H](NC(=O)[C@H]2CC3(C2)C[C@H](N2CCC(c4ccnc(N5CCN6c7cc(-c8ccccc8O)nnc7NC[C@@H]6C5)n4)CC2)C3)C(C)(C)C)cc1] |
| I-4 | 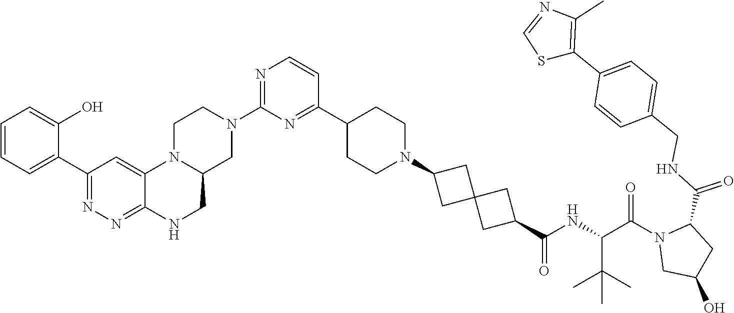 [SMILES: Cc1ncsc1-c1ccc(CNC(=O)[C@@H]2C[C@@H](O)CN2C(=O)[C@@H](NC(=O)[C@H]2CC3(C2)C[C@H](N2CCC(c4ccnc(N5CCN6c7cc(-c8ccccc8O)nnc7NC[C@@H]6C5)n4)CC2)C3)C(C)(C)C)cc1] |
| I-5 | 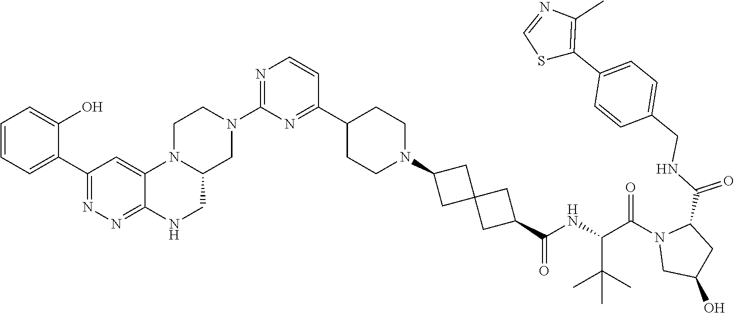 [SMILES: Cc1ncsc1-c1ccc(CNC(=O)[C@@H]2C[C@@H](O)CN2C(=O)[C@@H](NC(=O)[C@H]2CC3(C2)C[C@H](N2CCC(c4ccnc(N5CCN6c7cc(-c8ccccc8O)nnc7NC[C@H]6C5)n4)CC2)C3)C(C)(C)C)cc1] |
| I-7 | 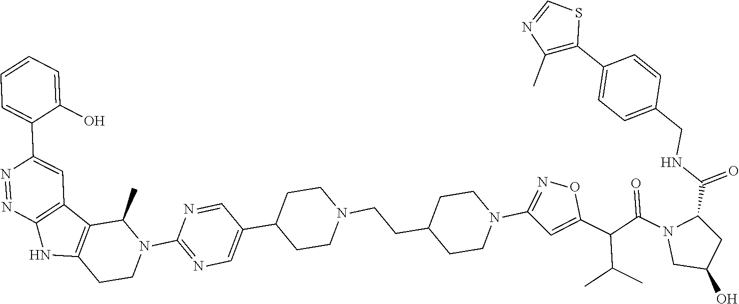 [SMILES: Cc1ncsc1-c1ccc(CNC(=O)[C@@H]2C[C@@H](O)CN2C(=O)C(c2cc(N3CCC(CCN4CCC(c5cnc(N6CCc7[nH]c8nnc(-c9ccccc9O)cc8c7[C@H]6C)nc5)CC4)CC3)no2)C(C)C)cc1] |
| I-8 | 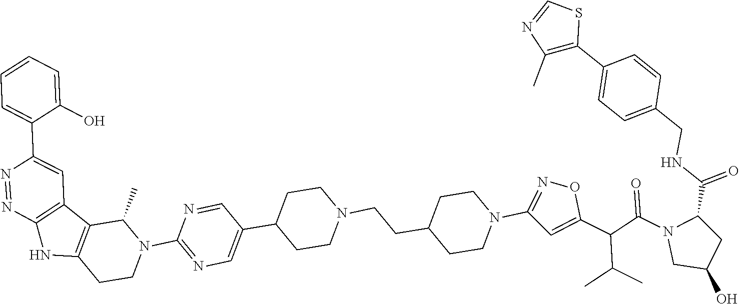 [SMILES: Cc1ncsc1-c1ccc(CNC(=O)[C@@H]2C[C@@H](O)CN2C(=O)C(c2cc(N3CCC(CCN4CCC(c5cnc(N6CCc7[nH]c8nnc(-c9ccccc9O)cc8c7[C@@H]6C)nc5)CC4)CC3)no2)C(C)C)cc1] |
| I-9 | 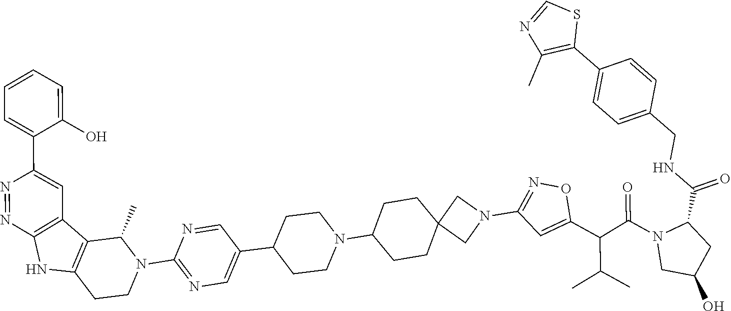 [SMILES: Cc1ncsc1-c1ccc(CNC(=O)[C@@H]2C[C@@H](O)CN2C(=O)C(c2cc(N3CC4(CCC(N5CCC(c6cnc(N7CCc8[nH]c9nnc(-c%10ccccc%10O)cc9c8[C@@H]7C)nc6)CC5)CC4)C3)no2)C(C)C)cc1] |
| I-10 | 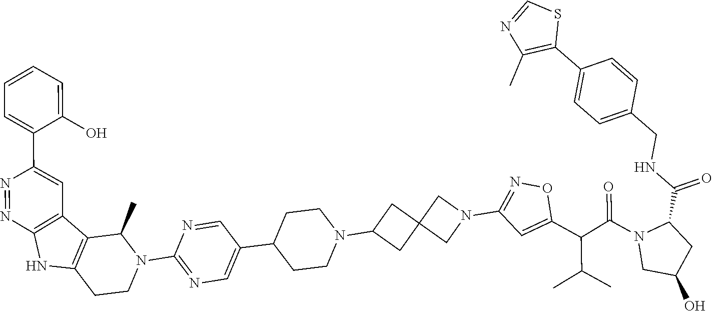 [SMILES: Cc1ncsc1-c1ccc(CNC(=O)[C@@H]2C[C@@H](O)CN2C(=O)C(c2cc(N3CC4(CC(N5CCC(c6cnc(N7CCc8[nH]c9nnc(-c%10ccccc%10O)cc9c8[C@H]7C)nc6)CC5)C4)C3)no2)C(C)C)cc1] |
| I-11 | 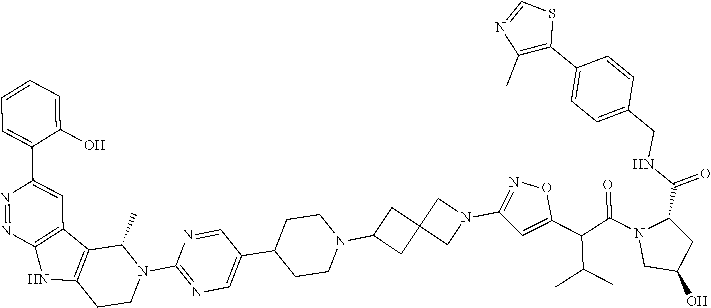 [SMILES: Cc1ncsc1-c1ccc(CNC(=O)[C@@H]2C[C@@H](O)CN2C(=O)C(c2cc(N3CC4(CC(N5CCC(c6cnc(N7CCc8[nH]c9nnc(-c%10ccccc%10O)cc9c8[C@@H]7C)nc6)CC5)C4)C3)no2)C(C)C)cc1] |
| I-12 | 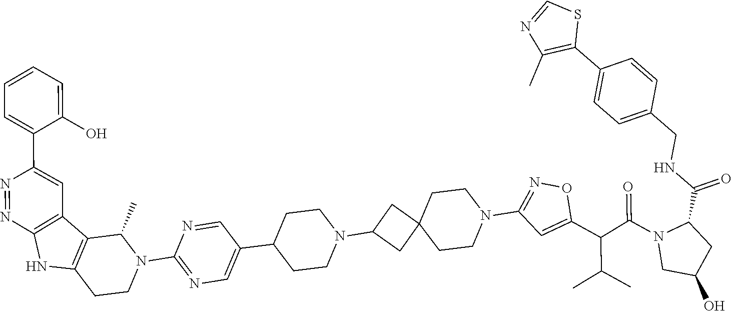 [SMILES: Cc1ncsc1-c1ccc(CNC(=O)[C@@H]2C[C@@H](O)CN2C(=O)C(c2cc(N3CCC4(CC3)CC(N3CCC(c5cnc(N6CCc7[nH]c8nnc(-c9ccccc9O)cc8c7[C@@H]6C)nc5)CC3)C4)no2)C(C)C)cc1] |
| I-13 | 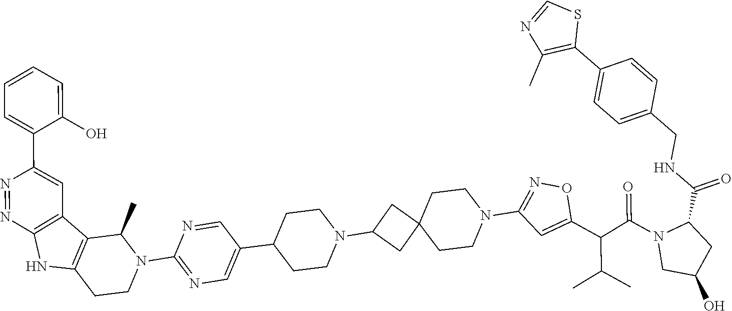 [SMILES: Cc1ncsc1-c1ccc(CNC(=O)[C@@H]2C[C@@H](O)CN2C(=O)C(c2cc(N3CCC4(CC3)CC(N3CCC(c5cnc(N6CCc7[nH]c8nnc(-c9ccccc9O)cc8c7[C@H]6C)nc5)CC3)C4)no2)C(C)C)cc1] |
| I-14 | 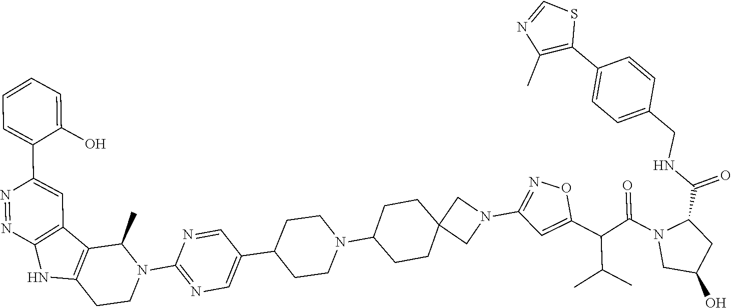 [SMILES: Cc1ncsc1-c1ccc(CNC(=O)[C@@H]2C[C@@H](O)CN2C(=O)C(c2cc(N3CC4(CCC(N5CCC(c6cnc(N7CCc8[nH]c9nnc(-c%10ccccc%10O)cc9c8[C@H]7C)nc6)CC5)CC4)C3)no2)C(C)C)cc1] |
| I-15 | 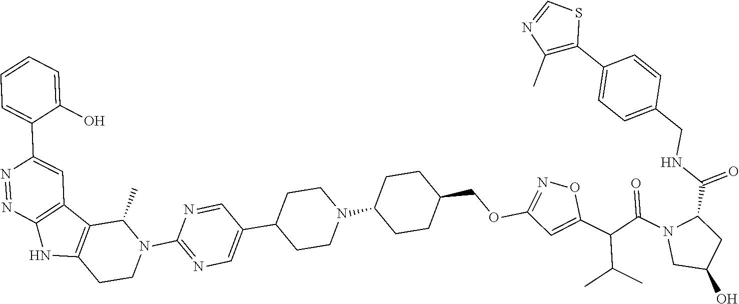 [SMILES: Cc1ncsc1-c1ccc(CNC(=O)[C@@H]2C[C@@H](O)CN2C(=O)C(c2cc(OC[C@H]3CC[C@H](N4CCC(c5cnc(N6CCc7[nH]c8nnc(-c9ccccc9O)cc8c7[C@@H]6C)nc5)CC4)CC3)no2)C(C)C)cc1] |
| I-16 | 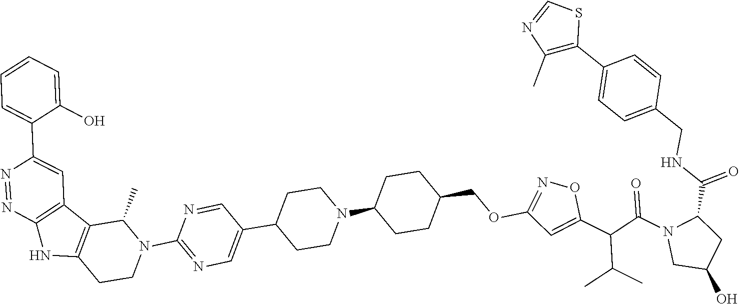 [SMILES: Cc1ncsc1-c1ccc(CNC(=O)[C@@H]2C[C@@H](O)CN2C(=O)C(c2cc(OC[C@H]3CC[C@@H](N4CCC(c5cnc(N6CCc7[nH]c8nnc(-c9ccccc9O)cc8c7[C@@H]6C)nc5)CC4)CC3)no2)C(C)C)cc1] |
| I-17 | 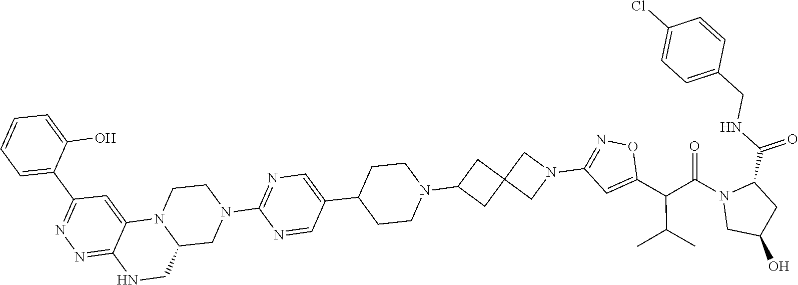 [SMILES: CC(C)C(C(=O)N1C[C@H](O)C[C@H]1C(=O)NCc1ccc(Cl)cc1)c1cc(N2CC3(CC(N4CCC(c5cnc(N6CCN7c8cc(-c9ccccc9O)nnc8NC[C@H]7C6)nc5)CC4)C3)C2)no1] |
| I-18 | 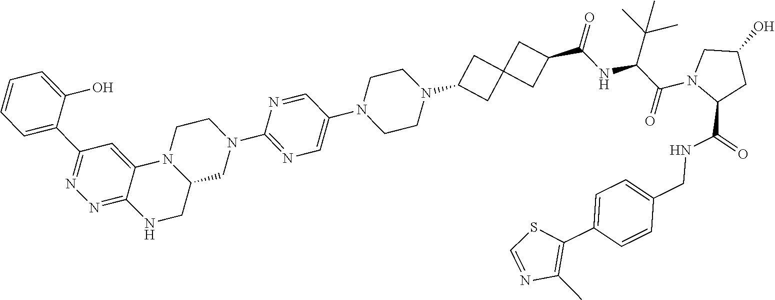 [SMILES: Cc1ncsc1-c1ccc(CNC(=O)[C@@H]2C[C@@H](O)CN2C(=O)[C@@H](NC(=O)[C@H]2CC3(C2)C[C@H](N2CCN(c4cnc(N5CCN6c7cc(-c8ccccc8O)nnc7NC[C@H]6C5)nc4)CC2)C3)C(C)(C)C)cc1] |
| I-19 | 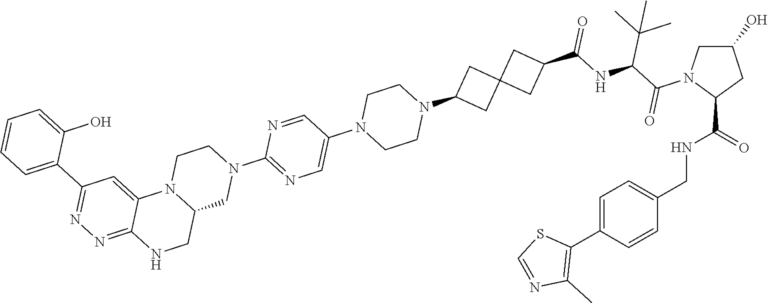 [SMILES: Cc1ncsc1-c1ccc(CNC(=O)[C@@H]2C[C@@H](O)CN2C(=O)[C@@H](NC(=O)[C@H]2CC3(C2)C[C@H](N2CCN(c4cnc(N5CCN6c7cc(-c8ccccc8O)nnc7NC[C@H]6C5)nc4)CC2)C3)C(C)(C)C)cc1] |
| I-20 | 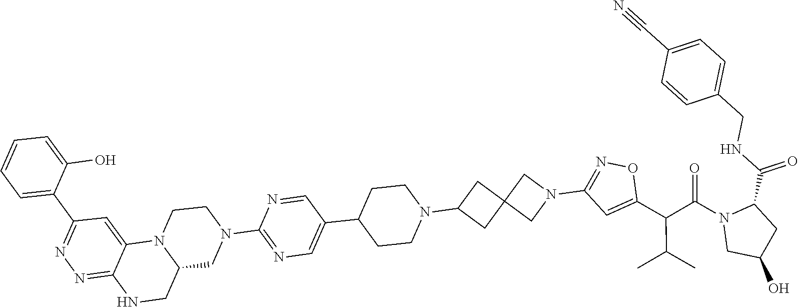 [SMILES: CC(C)C(C(=O)N1C[C@H](O)C[C@H]1C(=O)NCc1ccc(C#N)cc1)c1cc(N2CC3(CC(N4CCC(c5cnc(N6CCN7c8cc(-c9ccccc9O)nnc8NC[C@H]7C6)nc5)CC4)C3)C2)no1] |
| I-21 | 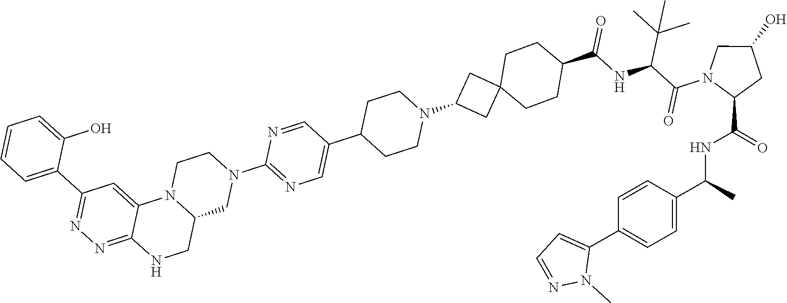 [SMILES: C[C@H](NC(=O)[C@@H]1C[C@@H](O)CN1C(=O)[C@@H](NC(=O)[C@H]1CCC2(CC1)C[C@H](N1CCC(c3cnc(N4CCN5c6cc(-c7ccccc7O)nnc6NC[C@H]5C4)nc3)CC1)C2)C(C)(C)C)c1ccc(-c2ccnn2C)cc1] |
| I-22 | 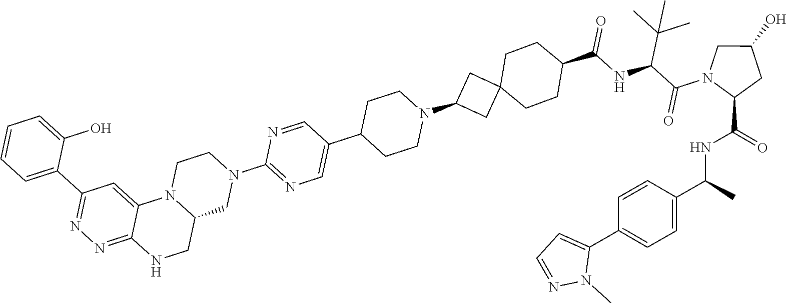 [SMILES: C[C@H](NC(=O)[C@@H]1C[C@@H](O)CN1C(=O)[C@@H](NC(=O)[C@H]1CCC2(CC1)C[C@H](N1CCC(c3cnc(N4CCN5c6cc(-c7ccccc7O)nnc6NC[C@H]5C4)nc3)CC1)C2)C(C)(C)C)c1ccc(-c2ccnn2C)cc1] |
| I-23 | 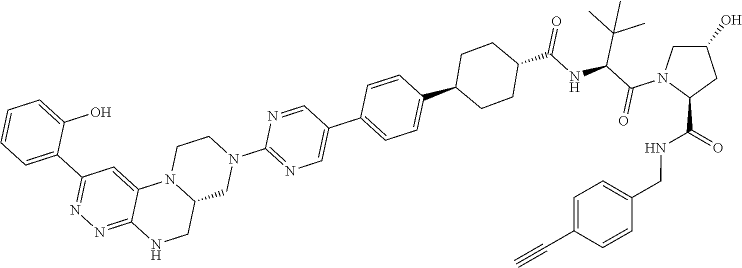 [SMILES: C#Cc1ccc(CNC(=O)[C@@H]2C[C@@H](O)CN2C(=O)[C@@H](NC(=O)[C@H]2CC[C@H](c3ccc(-c4cnc(N5CCN6c7cc(-c8ccccc8O)nnc7NC[C@H]6C5)nc4)cc3)CC2)C(C)(C)C)cc1] |
| I-24 | 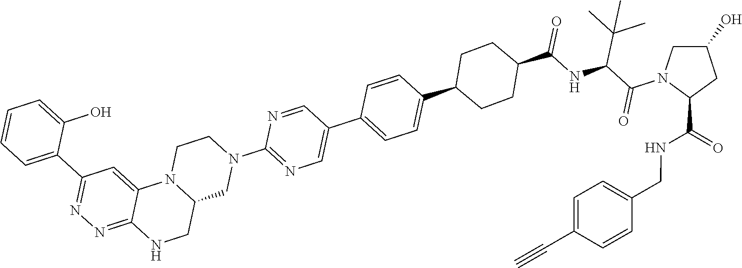 [SMILES: C#Cc1ccc(CNC(=O)[C@@H]2C[C@@H](O)CN2C(=O)[C@@H](NC(=O)[C@H]2CC[C@@H](c3ccc(-c4cnc(N5CCN6c7cc(-c8ccccc8O)nnc7NC[C@H]6C5)nc4)cc3)CC2)C(C)(C)C)cc1] |
| I-25 | 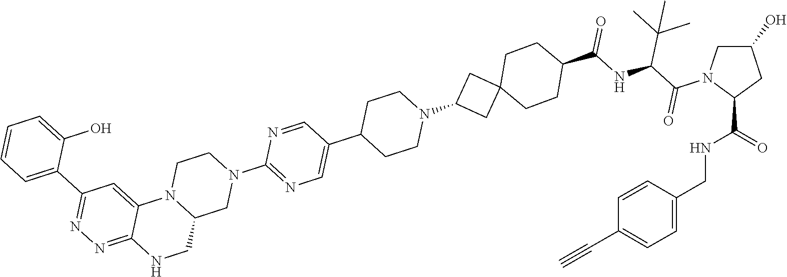 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)cc1] |
| I-26 | 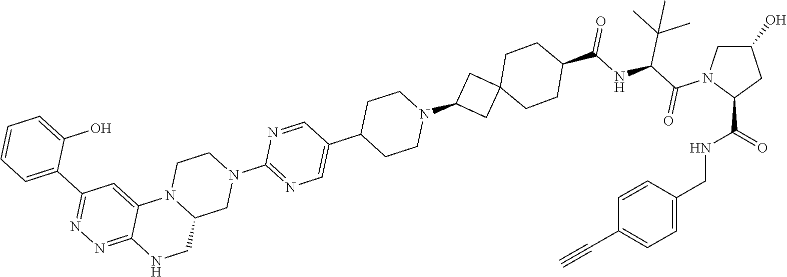 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)cc1] |
| I-27 | 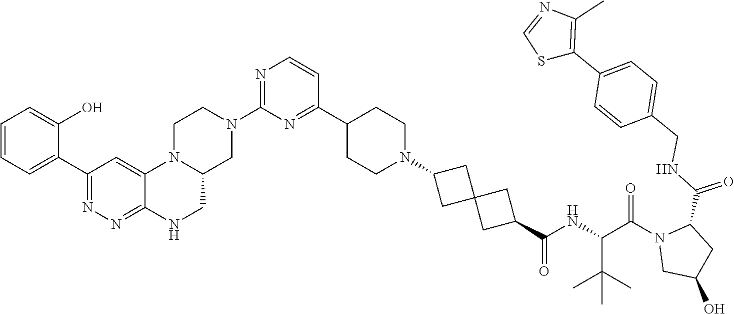 [SMILES: Cc1ncsc1-c1ccc(CNC(=O)[C@@H]2C[C@@H](O)CN2C(=O)[C@@H](NC(=O)[C@H]2CC3(C2)C[C@H](N2CCC(c4ccnc(N5CCN6c7cc(-c8ccccc8O)nnc7NC[C@H]6C5)n4)CC2)C3)C(C)(C)C)cc1] |
| I-28 | 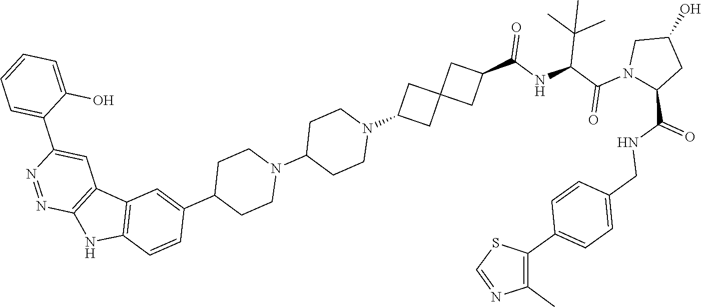 [SMILES: Cc1ncsc1-c1ccc(CNC(=O)[C@@H]2C[C@@H](O)CN2C(=O)[C@@H](NC(=O)[C@H]2CC3(C2)C[C@H](N2CCC(N4CCC(c5ccc6[nH]c7nnc(-c8ccccc8O)cc7c6c5)CC4)CC2)C3)C(C)(C)C)cc1] |
| I-29 | 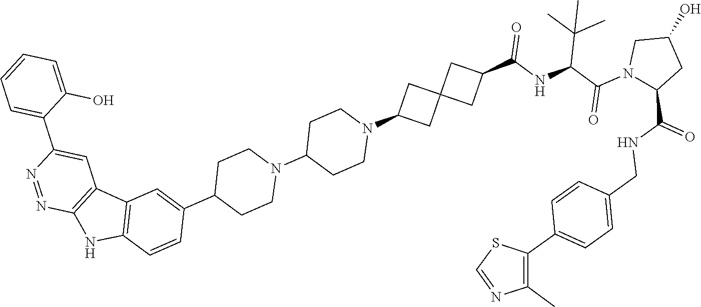 [SMILES: Cc1ncsc1-c1ccc(CNC(=O)[C@@H]2C[C@@H](O)CN2C(=O)[C@@H](NC(=O)[C@H]2CC3(C2)C[C@H](N2CCC(N4CCC(c5ccc6[nH]c7nnc(-c8ccccc8O)cc7c6c5)CC4)CC2)C3)C(C)(C)C)cc1] |
| I-30 | 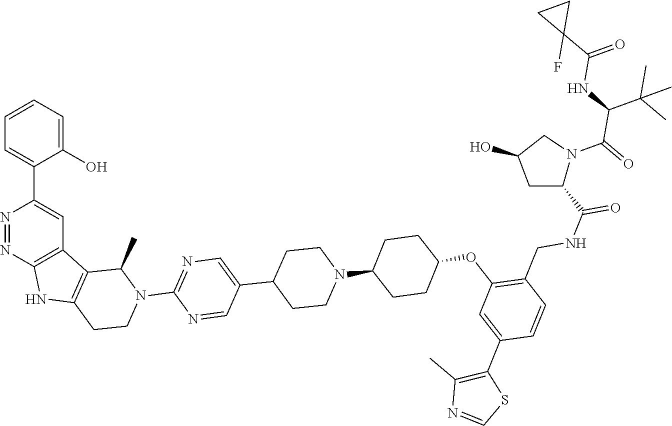 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@H](N3CCC(c4cnc(N5CCc6[nH]c7nnc(-c8ccccc8O)cc7c6[C@H]5C)nc4)CC3)CC2)c1] |
| I-31 | 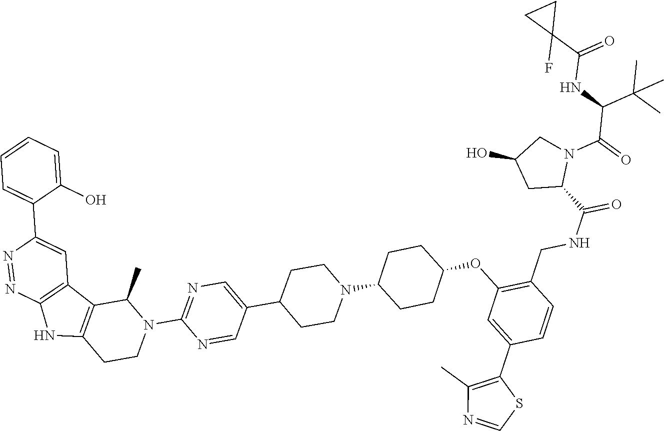 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@@H](N3CCC(c4cnc(N5CCc6[nH]c7nnc(-c8ccccc8O)cc7c6[C@H]5C)nc4)CC3)CC2)c1] |
| I-32 | 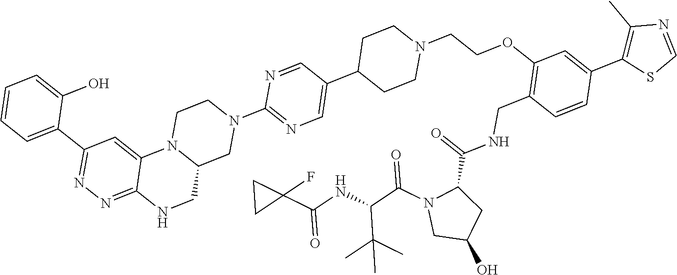 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCC(c3cnc(N4CCN5c6cc(-c7ccccc7O)nnc6NC[C@H]5C4)nc3)CC2)c1] |
| I-33 | 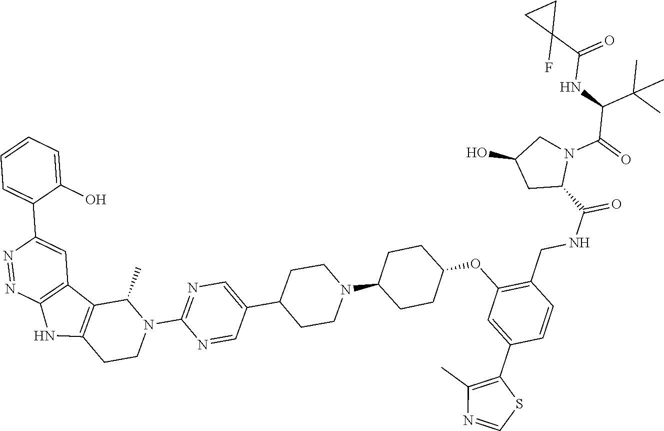 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@H](N3CCC(c4cnc(N5CCc6[nH]c7nnc(-c8ccccc8O)cc7c6[C@@H]5C)nc4)CC3)CC2)c1] |
| I-34 | 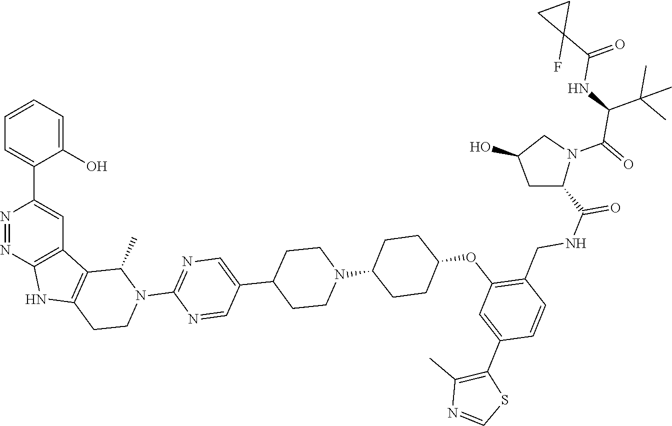 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@@H](N3CCC(c4cnc(N5CCc6[nH]c7nnc(-c8ccccc8O)cc7c6[C@@H]5C)nc4)CC3)CC2)c1] |
| I-35 | 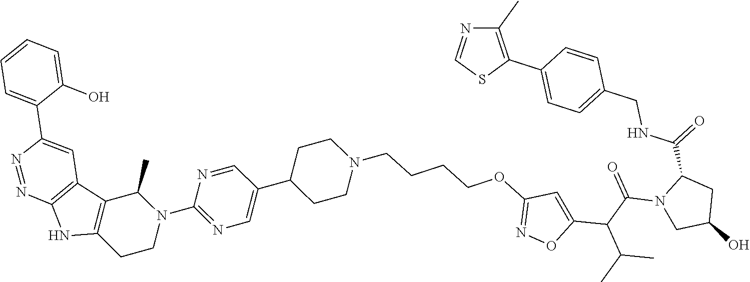 [SMILES: Cc1ncsc1-c1ccc(CNC(=O)[C@@H]2C[C@@H](O)CN2C(=O)C(c2cc(OCCCCN3CCC(c4cnc(N5CCc6[nH]c7nnc(-c8ccccc8O)cc7c6[C@H]5C)nc4)CC3)no2)C(C)C)cc1] |
| I-36 | 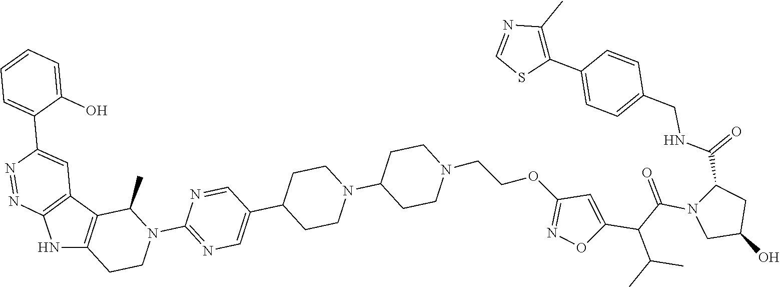 [SMILES: Cc1ncsc1-c1ccc(CNC(=O)[C@@H]2C[C@@H](O)CN2C(=O)C(c2cc(OCCN3CCC(N4CCC(c5cnc(N6CCc7[nH]c8nnc(-c9ccccc9O)cc8c7[C@H]6C)nc5)CC4)CC3)no2)C(C)C)cc1] |
| I-37 | 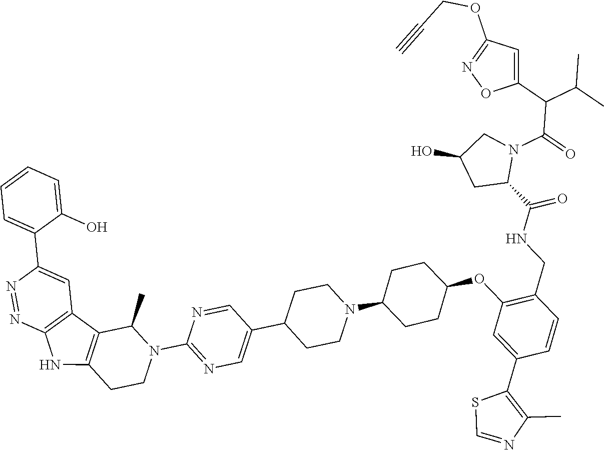 [SMILES: C#CCOc1cc(C(C(=O)N2C[C@H](O)C[C@H]2C(=O)NCc2ccc(-c3scnc3C)cc2O[C@H]2CC[C@@H](N3CCC(c4cnc(N5CCc6[nH]c7nnc(-c8ccccc8O)cc7c6[C@H]5C)nc4)CC3)CC2)C(C)C)on1] |
| I-38 | 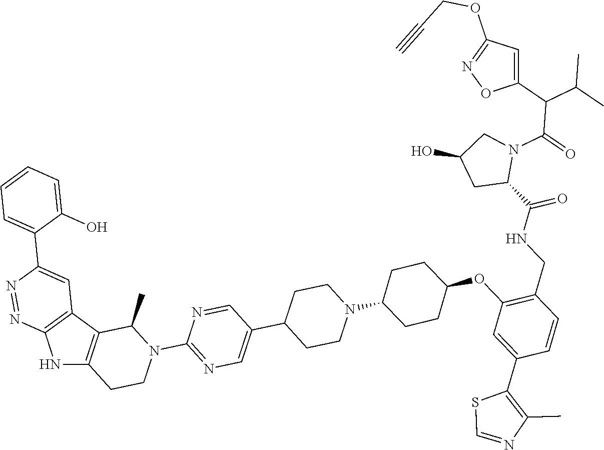 [SMILES: C#CCOc1cc(C(C(=O)N2C[C@H](O)C[C@H]2C(=O)NCc2ccc(-c3scnc3C)cc2O[C@H]2CC[C@H](N3CCC(c4cnc(N5CCc6[nH]c7nnc(-c8ccccc8O)cc7c6[C@H]5C)nc4)CC3)CC2)C(C)C)on1] |
| I-39 | 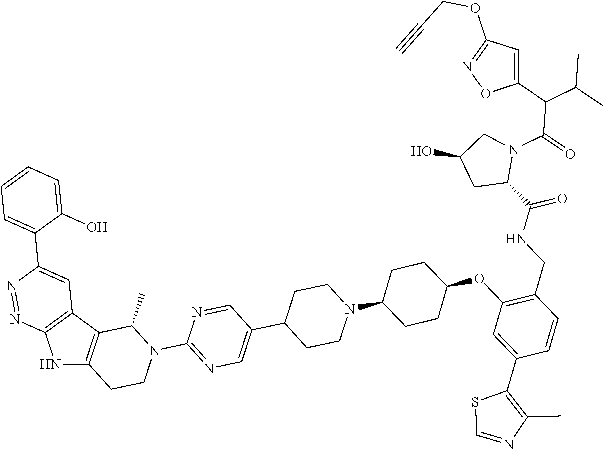 [SMILES: C#CCOc1cc(C(C(=O)N2C[C@H](O)C[C@H]2C(=O)NCc2ccc(-c3scnc3C)cc2O[C@H]2CC[C@@H](N3CCC(c4cnc(N5CCc6[nH]c7nnc(-c8ccccc8O)cc7c6[C@@H]5C)nc4)CC3)CC2)C(C)C)on1] |
| I-40 | 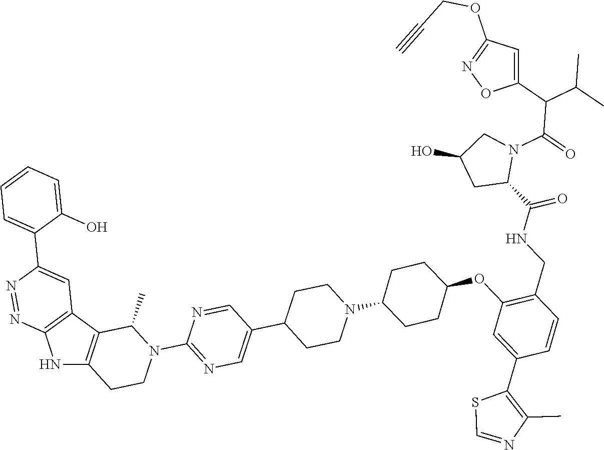 [SMILES: C#CCOc1cc(C(C(=O)N2C[C@H](O)C[C@H]2C(=O)NCc2ccc(-c3scnc3C)cc2O[C@H]2CC[C@H](N3CCC(c4cnc(N5CCc6[nH]c7nnc(-c8ccccc8O)cc7c6[C@@H]5C)nc4)CC3)CC2)C(C)C)on1] |
| I-41 | 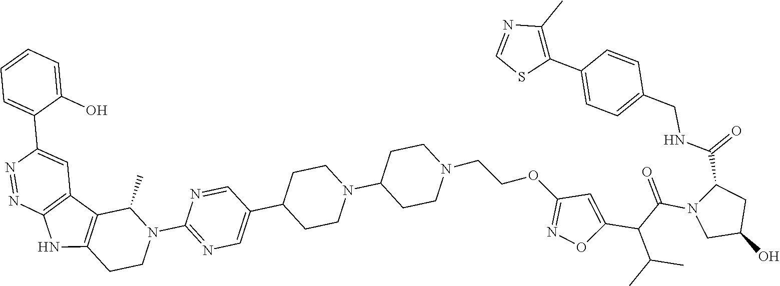 [SMILES: Cc1ncsc1-c1ccc(CNC(=O)[C@@H]2C[C@@H](O)CN2C(=O)C(c2cc(OCCN3CCC(N4CCC(c5cnc(N6CCc7[nH]c8nnc(-c9ccccc9O)cc8c7[C@@H]6C)nc5)CC4)CC3)no2)C(C)C)cc1] |
| I-42 | 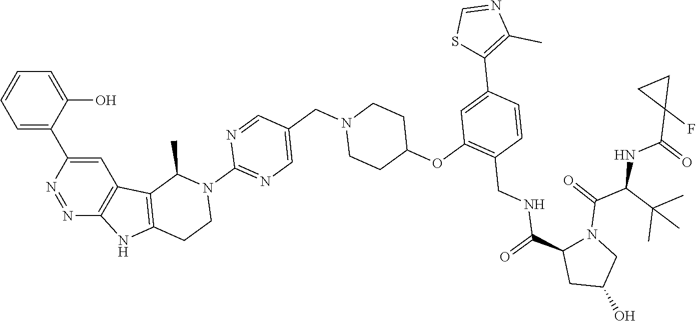 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CCN(Cc3cnc(N4CCc5[nH]c6nnc(-c7ccccc7O)cc6c5[C@H]4C)nc3)CC2)c1] |
| I-43 | 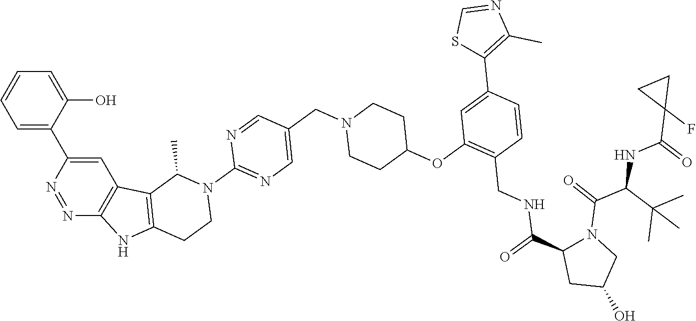 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CCN(Cc3cnc(N4CCc5[nH]c6nnc(-c7ccccc7O)cc6c5[C@@H]4C)nc3)CC2)c1] |
| I-44 | 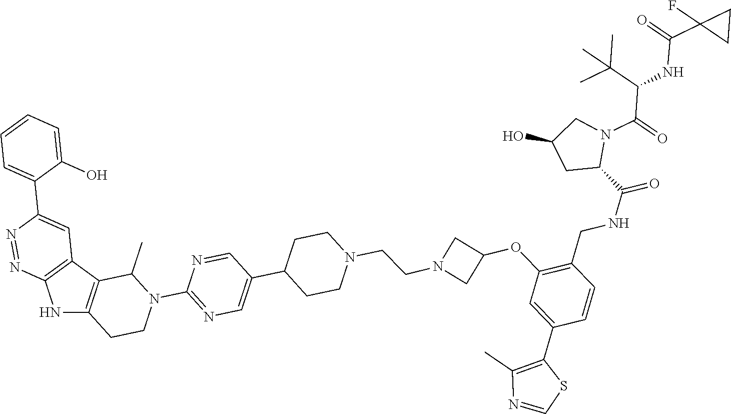 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CN(CCN3CCC(c4cnc(N5CCc6[nH]c7nnc(-c8ccccc8O)cc7c6C5C)nc4)CC3)C2)c1] |
| I-45 | 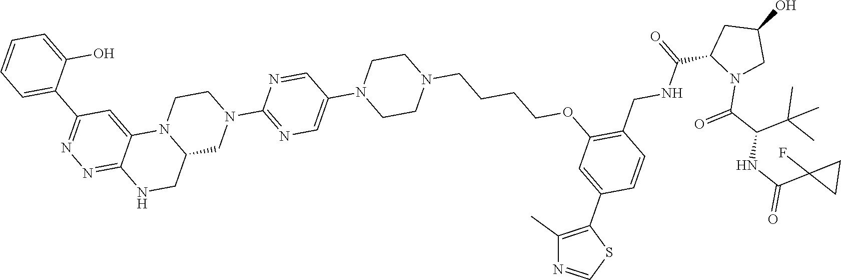 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCN2CCN(c3cnc(N4CCN5c6cc(-c7ccccc7O)nnc6NC[C@H]5C4)nc3)CC2)c1] |
| I-46 | 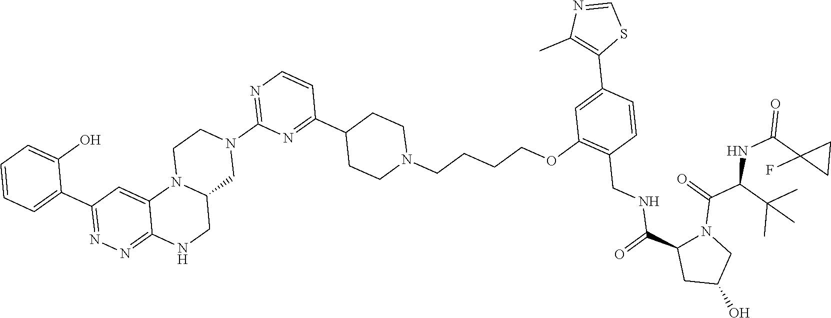 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCN2CCC(c3ccnc(N4CCN5c6cc(-c7ccccc7O)nnc6NC[C@H]5C4)n3)CC2)c1] |
| I-47 | 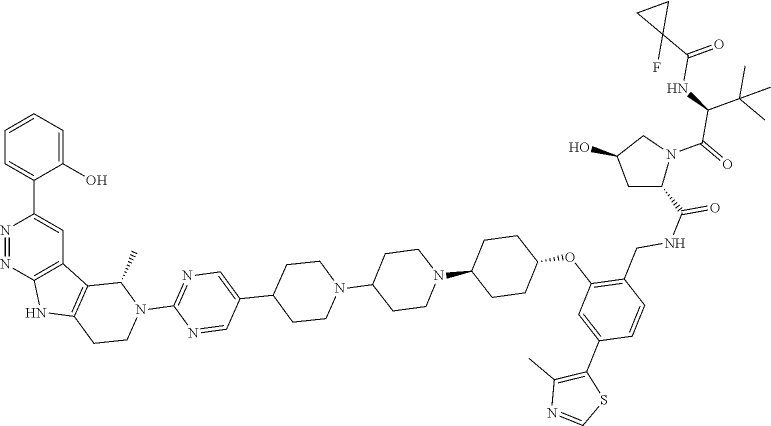 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@H](N3CCC(N4CCC(c5cnc(N6CCc7[nH]c8nnc(-c9ccccc9O)cc8c7[C@@H]6C)nc5)CC4)CC3)CC2)c1] |
| I-48 | 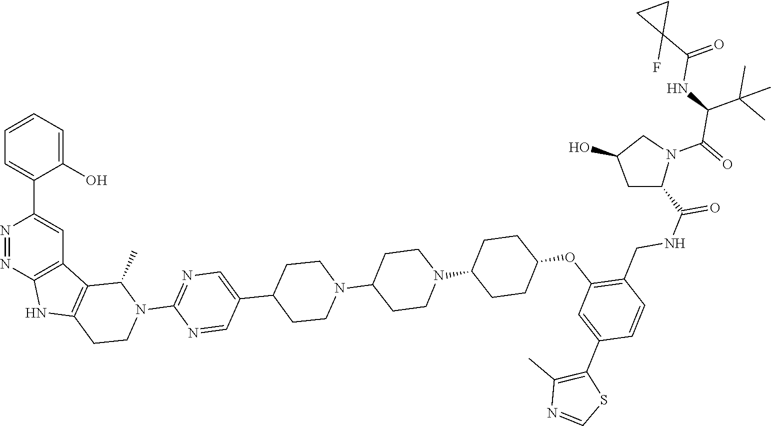 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@@H](N3CCC(N4CCC(c5cnc(N6CCc7[nH]c8nnc(-c9ccccc9O)cc8c7[C@@H]6C)nc5)CC4)CC3)CC2)c1] |
| I-49 | 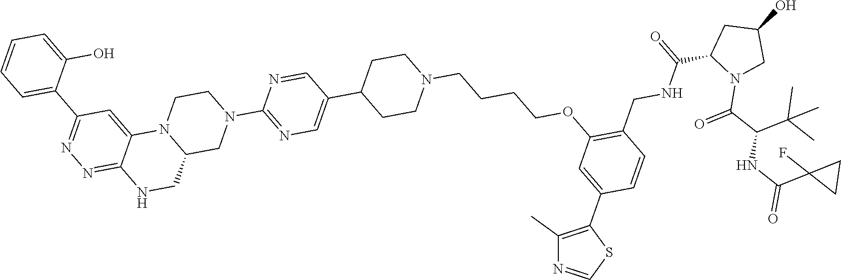 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCN2CCC(c3cnc(N4CCN5c6cc(-c7ccccc7O)nnc6NC[C@H]5C4)nc3)CC2)c1] |
| I-50 | 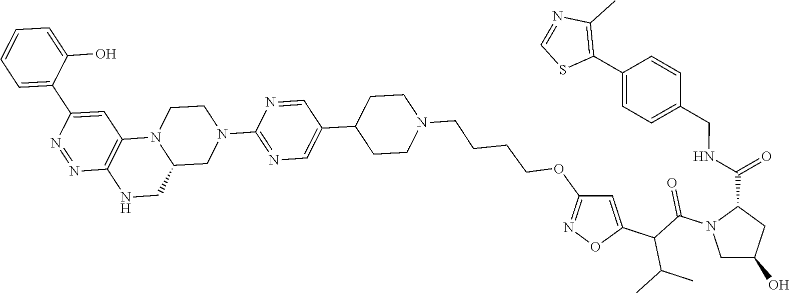 [SMILES: Cc1ncsc1-c1ccc(CNC(=O)[C@@H]2C[C@@H](O)CN2C(=O)C(c2cc(OCCCCN3CCC(c4cnc(N5CCN6c7cc(-c8ccccc8O)nnc7NC[C@H]6C5)nc4)CC3)no2)C(C)C)cc1] |
| I-51 | 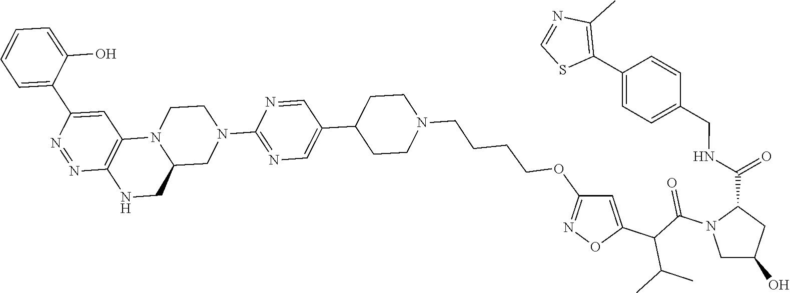 [SMILES: Cc1ncsc1-c1ccc(CNC(=O)[C@@H]2C[C@@H](O)CN2C(=O)C(c2cc(OCCCCN3CCC(c4cnc(N5CCN6c7cc(-c8ccccc8O)nnc7NC[C@@H]6C5)nc4)CC3)no2)C(C)C)cc1] |
| I-52 | 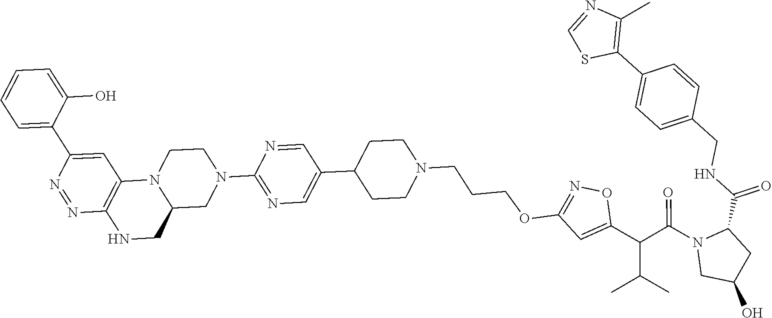 [SMILES: Cc1ncsc1-c1ccc(CNC(=O)[C@@H]2C[C@@H](O)CN2C(=O)C(c2cc(OCCCN3CCC(c4cnc(N5CCN6c7cc(-c8ccccc8O)nnc7NC[C@@H]6C5)nc4)CC3)no2)C(C)C)cc1] |
| I-53 | 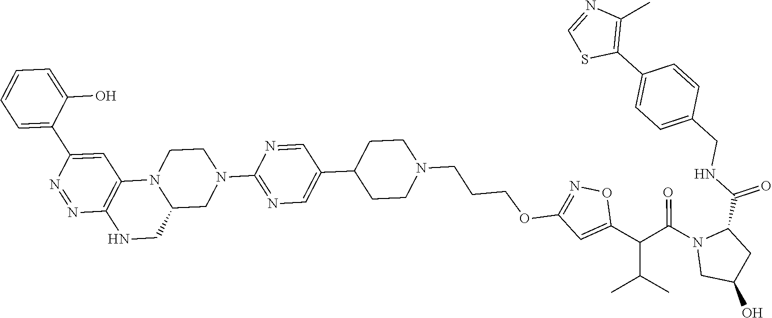 [SMILES: Cc1ncsc1-c1ccc(CNC(=O)[C@@H]2C[C@@H](O)CN2C(=O)C(c2cc(OCCCN3CCC(c4cnc(N5CCN6c7cc(-c8ccccc8O)nnc7NC[C@H]6C5)nc4)CC3)no2)C(C)C)cc1] |
| I-54 | 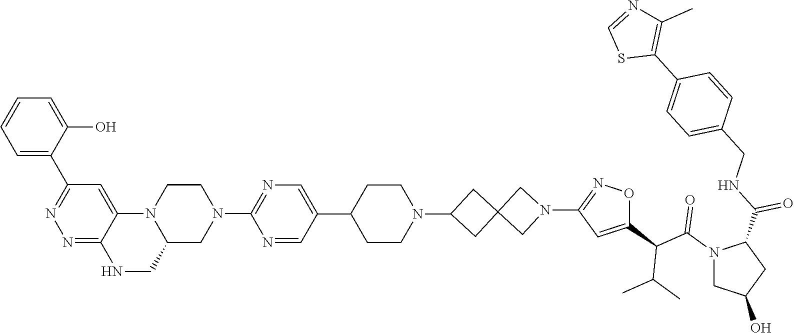 [SMILES: Cc1ncsc1-c1ccc(CNC(=O)[C@@H]2C[C@@H](O)CN2C(=O)[C@H](c2cc(N3CC4(CC(N5CCC(c6cnc(N7CCN8c9cc(-c%10ccccc%10O)nnc9NC[C@H]8C7)nc6)CC5)C4)C3)no2)C(C)C)cc1] |
| I-55 | 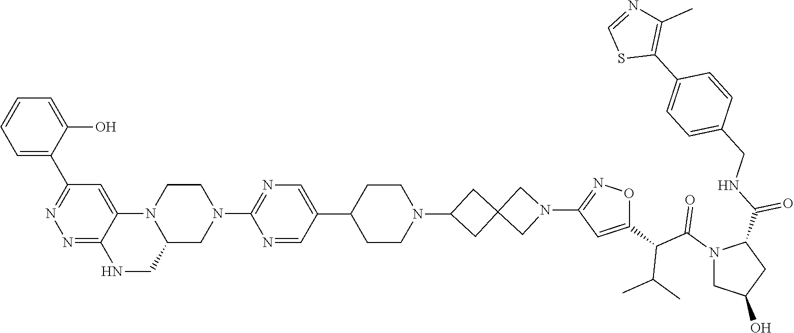 [SMILES: Cc1ncsc1-c1ccc(CNC(=O)[C@@H]2C[C@@H](O)CN2C(=O)[C@@H](c2cc(N3CC4(CC(N5CCC(c6cnc(N7CCN8c9cc(-c%10ccccc%10O)nnc9NC[C@H]8C7)nc6)CC5)C4)C3)no2)C(C)C)cc1] |
| I-56 | 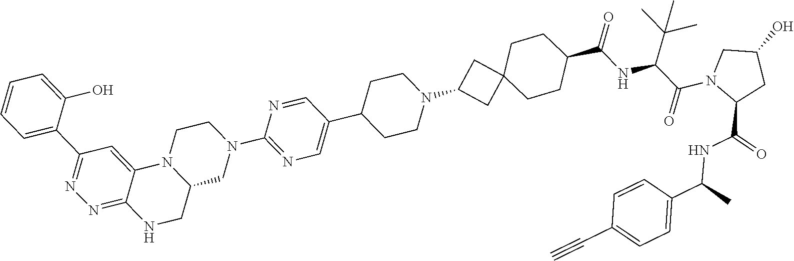 [SMILES: C#Cc1ccc([C@H](C)NC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)cc1] |
| I-57 | 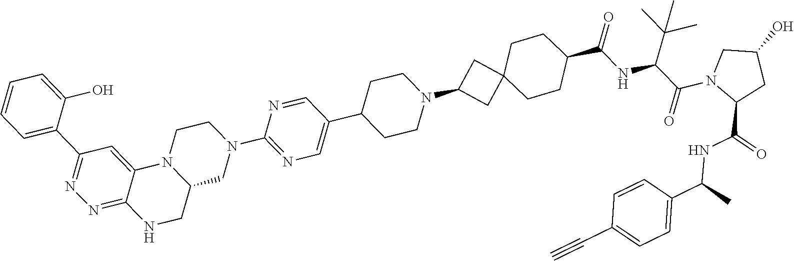 [SMILES: C#Cc1ccc([C@H](C)NC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)cc1] |
| I-58 | 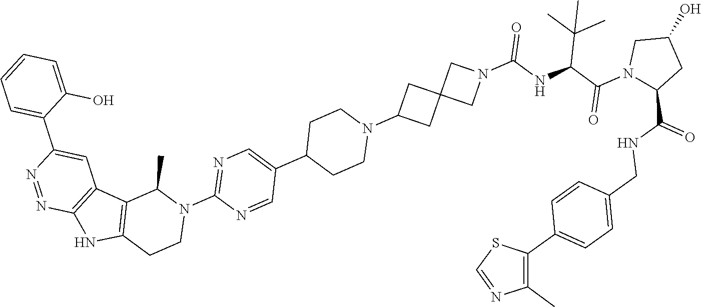 [SMILES: Cc1ncsc1-c1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-59 | 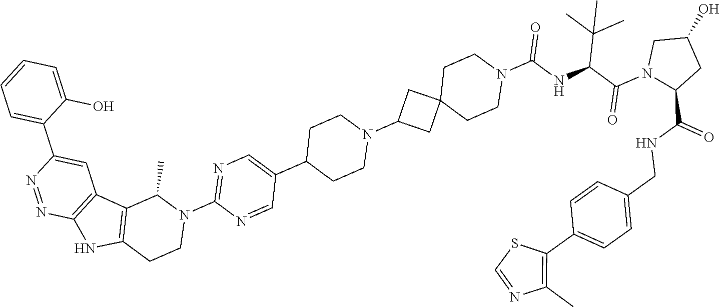 [SMILES: Cc1ncsc1-c1ccc(CNC(=O)[C@@H]2C[C@@H](O)CN2C(=O)[C@@H](NC(=O)N2CCC3(CC2)CC(N2CCC(c4cnc(N5CCc6[nH]c7nnc(-c8ccccc8O)cc7c6[C@@H]5C)nc4)CC2)C3)C(C)(C)C)cc1] |
| I-62 | 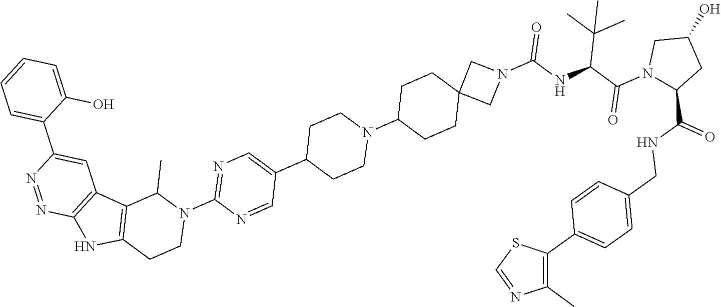 [SMILES: Cc1ncsc1-c1ccc(CNC(=O)[C@@H]2C[C@@H](O)CN2C(=O)[C@@H](NC(=O)N2CC3(CCC(N4CCC(c5cnc(N6CCc7[nH]c8nnc(-c9ccccc9O)cc8c7C6C)nc5)CC4)CC3)C2)C(C)(C)C)cc1] |
| I-63 | 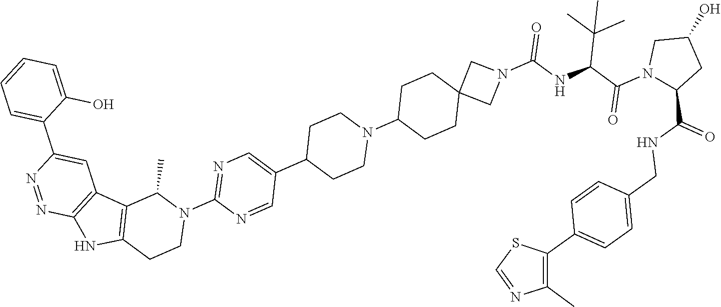 [SMILES: Cc1ncsc1-c1ccc(CNC(=O)[C@@H]2C[C@@H](O)CN2C(=O)[C@@H](NC(=O)N2CC3(CCC(N4CCC(c5cnc(N6CCc7[nH]c8nnc(-c9ccccc9O)cc8c7[C@@H]6C)nc5)CC4)CC3)C2)C(C)(C)C)cc1] |
| I-64 | 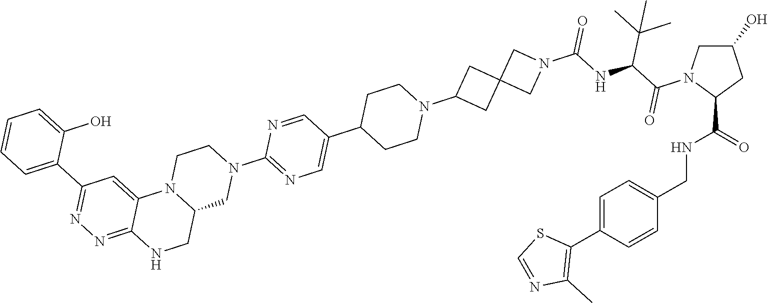 [SMILES: Cc1ncsc1-c1ccc(CNC(=O)[C@@H]2C[C@@H](O)CN2C(=O)[C@@H](NC(=O)N2CC3(CC(N4CCC(c5cnc(N6CCN7c8cc(-c9ccccc9O)nnc8NC[C@H]7C6)nc5)CC4)C3)C2)C(C)(C)C)cc1] |
| I-65 | 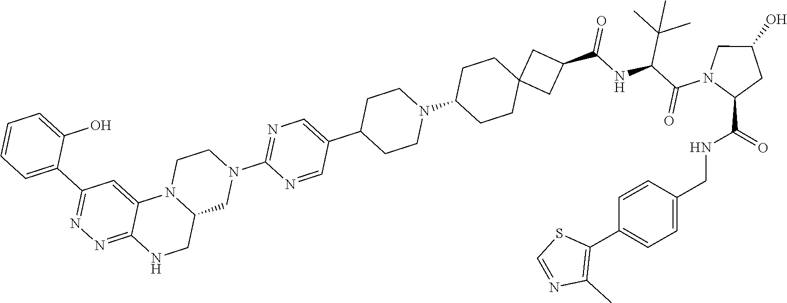 [SMILES: Cc1ncsc1-c1ccc(CNC(=O)[C@@H]2C[C@@H](O)CN2C(=O)[C@@H](NC(=O)[C@H]2CC3(CC[C@H](N4CCC(c5cnc(N6CCN7c8cc(-c9ccccc9O)nnc8NC[C@H]7C6)nc5)CC4)CC3)C2)C(C)(C)C)cc1] |
| I-66 | 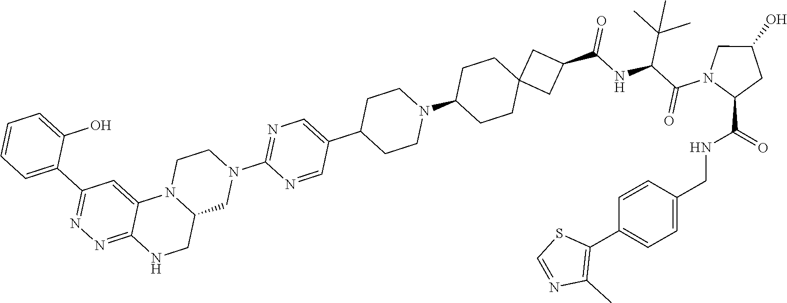 [SMILES: Cc1ncsc1-c1ccc(CNC(=O)[C@@H]2C[C@@H](O)CN2C(=O)[C@@H](NC(=O)[C@H]2CC3(CC[C@H](N4CCC(c5cnc(N6CCN7c8cc(-c9ccccc9O)nnc8NC[C@H]7C6)nc5)CC4)CC3)C2)C(C)(C)C)cc1] |
| I-67 | 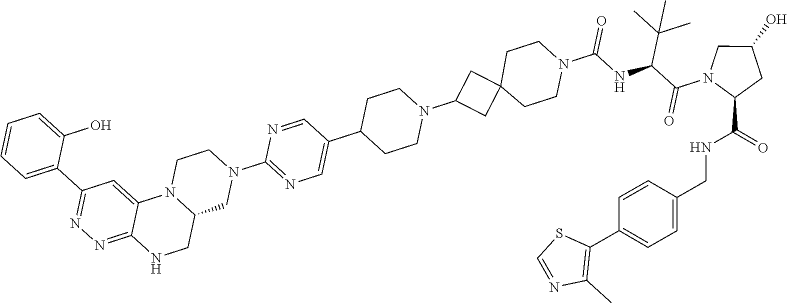 [SMILES: Cc1ncsc1-c1ccc(CNC(=O)[C@@H]2C[C@@H](O)CN2C(=O)[C@@H](NC(=O)N2CCC3(CC2)CC(N2CCC(c4cnc(N5CCN6c7cc(-c8ccccc8O)nnc7NC[C@H]6C5)nc4)CC2)C3)C(C)(C)C)cc1] |
| I-68 | 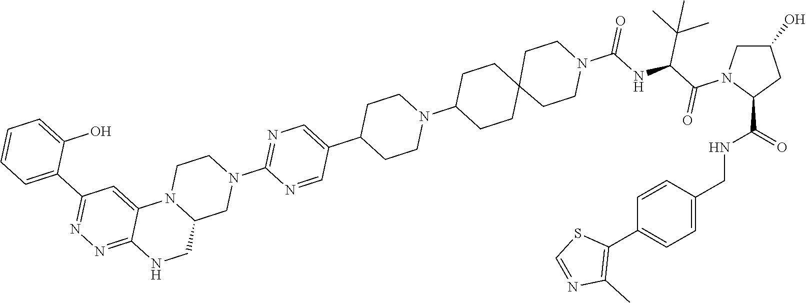 [SMILES: Cc1ncsc1-c1ccc(CNC(=O)[C@@H]2C[C@@H](O)CN2C(=O)[C@@H](NC(=O)N2CCC3(CCC(N4CCC(c5cnc(N6CCN7c8cc(-c9ccccc9O)nnc8NC[C@H]7C6)nc5)CC4)CC3)CC2)C(C)(C)C)cc1] |
| I-69 | 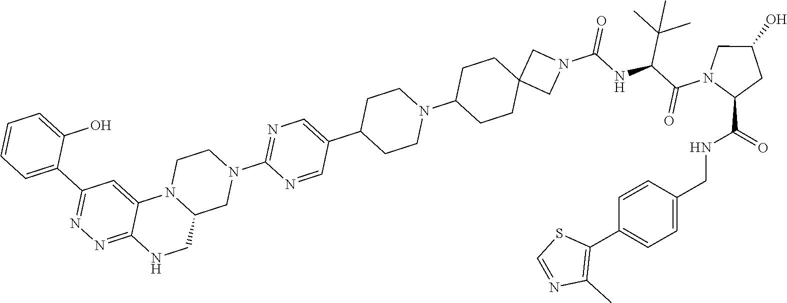 [SMILES: Cc1ncsc1-c1ccc(CNC(=O)[C@@H]2C[C@@H](O)CN2C(=O)[C@@H](NC(=O)N2CC3(CCC(N4CCC(c5cnc(N6CCN7c8cc(-c9ccccc9O)nnc8NC[C@H]7C6)nc5)CC4)CC3)C2)C(C)(C)C)cc1] |
| I-70 | 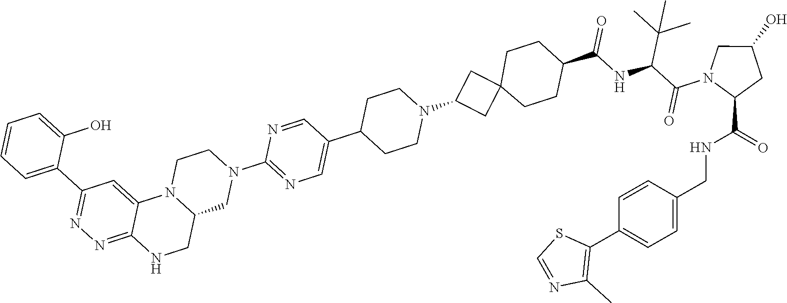 [SMILES: Cc1ncsc1-c1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)cc1] |
| I-71 | 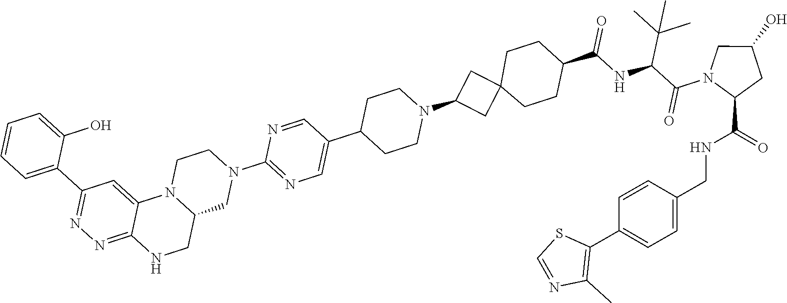 [SMILES: Cc1ncsc1-c1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)cc1] |
| I-72 | 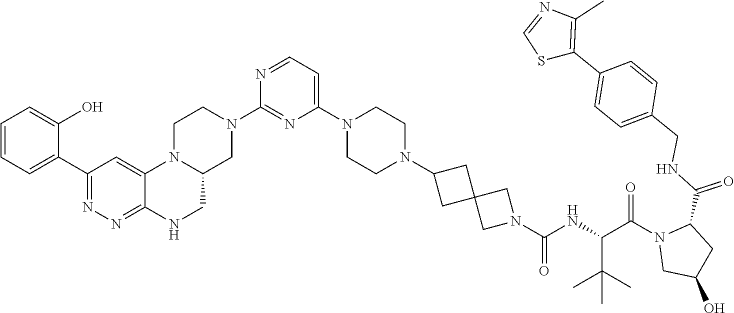 [SMILES: Cc1ncsc1-c1ccc(CNC(=O)[C@@H]2C[C@@H](O)CN2C(=O)[C@@H](NC(=O)N2CC3(CC(N4CCN(c5ccnc(N6CCN7c8cc(-c9ccccc9O)nnc8NC[C@H]7C6)n5)CC4)C3)C2)C(C)(C)C)cc1] |
| I-73 | 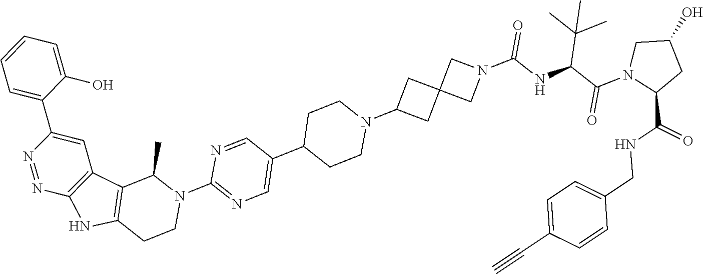 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-74 | 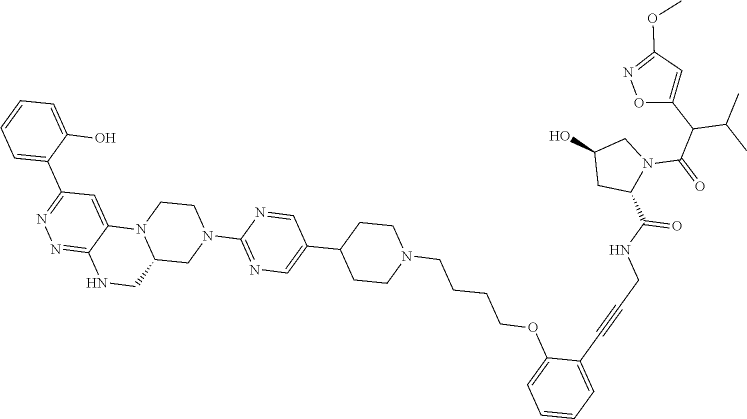 [SMILES: COc1cc(C(C(=O)N2C[C@H](O)C[C@H]2C(=O)NCC#Cc2ccccc2OCCCCN2CCC(c3cnc(N4CCN5c6cc(-c7ccccc7O)nnc6NC[C@H]5C4)nc3)CC2)C(C)C)on1] |
| I-75 | 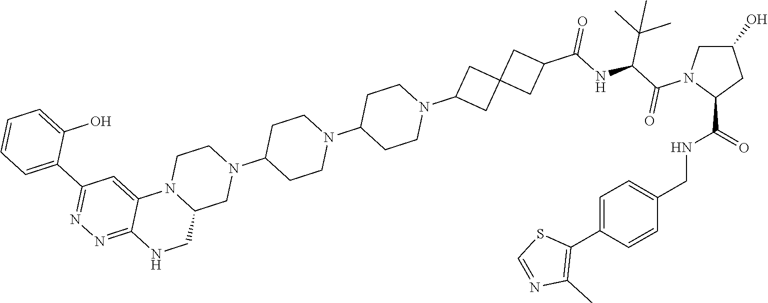 [SMILES: Cc1ncsc1-c1ccc(CNC(=O)[C@@H]2C[C@@H](O)CN2C(=O)[C@@H](NC(=O)C2CC3(C2)CC(N2CCC(N4CCC(N5CCN6c7cc(-c8ccccc8O)nnc7NC[C@H]6C5)CC4)CC2)C3)C(C)(C)C)cc1] |
| I-76 | 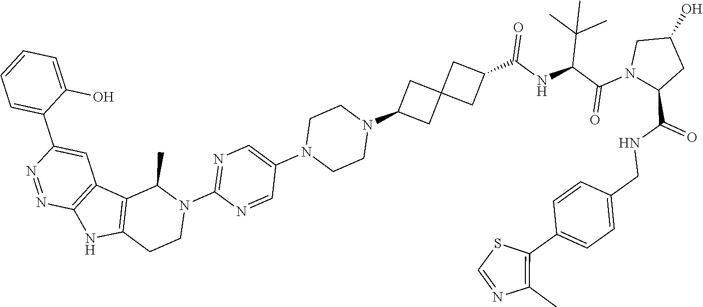 [SMILES: Cc1ncsc1-c1ccc(CNC(=O)[C@@H]2C[C@@H](O)CN2C(=O)[C@@H](NC(=O)[C@H]2CC3(C2)C[C@H](N2CCN(c4cnc(N5CCc6[nH]c7nnc(-c8ccccc8O)cc7c6[C@H]5C)nc4)CC2)C3)C(C)(C)C)cc1] |
| I-77 | 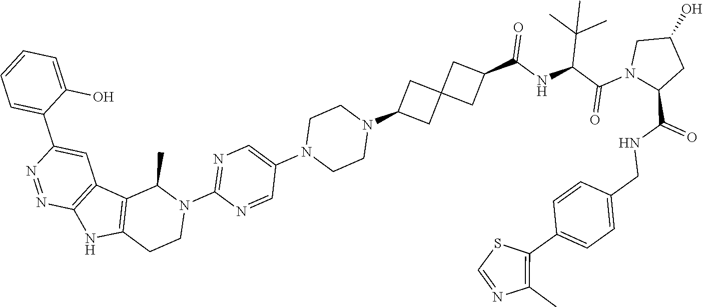 [SMILES: Cc1ncsc1-c1ccc(CNC(=O)[C@@H]2C[C@@H](O)CN2C(=O)[C@@H](NC(=O)[C@H]2CC3(C2)C[C@H](N2CCN(c4cnc(N5CCc6[nH]c7nnc(-c8ccccc8O)cc7c6[C@H]5C)nc4)CC2)C3)C(C)(C)C)cc1] |
| I-78 | 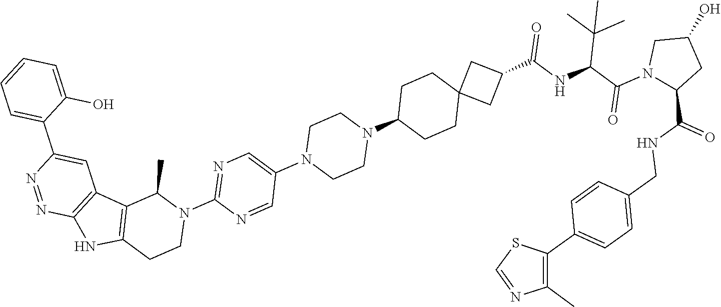 [SMILES: Cc1ncsc1-c1ccc(CNC(=O)[C@@H]2C[C@@H](O)CN2C(=O)[C@@H](NC(=O)[C@H]2CC3(CC[C@H](N4CCN(c5cnc(N6CCc7[nH]c8nnc(-c9ccccc9O)cc8c7[C@H]6C)nc5)CC4)CC3)C2)C(C)(C)C)cc1] |
| I-79 | 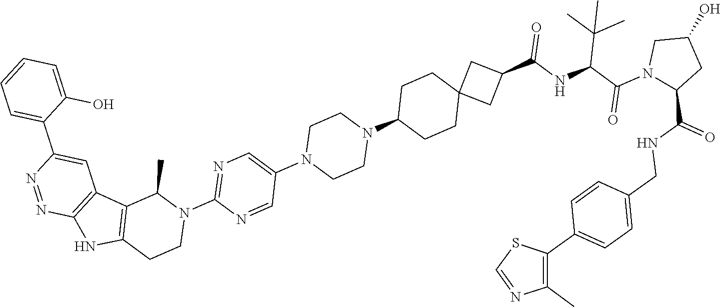 [SMILES: Cc1ncsc1-c1ccc(CNC(=O)[C@@H]2C[C@@H](O)CN2C(=O)[C@@H](NC(=O)[C@H]2CC3(CC[C@H](N4CCN(c5cnc(N6CCc7[nH]c8nnc(-c9ccccc9O)cc8c7[C@H]6C)nc5)CC4)CC3)C2)C(C)(C)C)cc1] |
| I-80 | 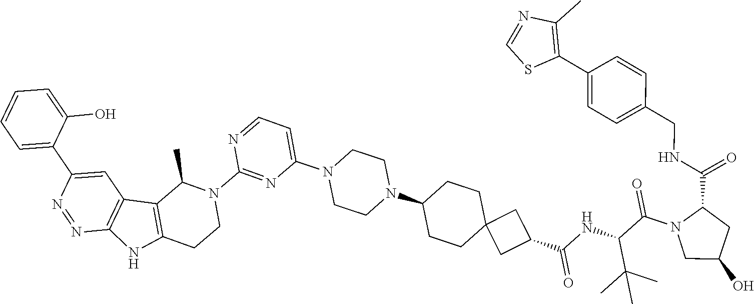 [SMILES: Cc1ncsc1-c1ccc(CNC(=O)[C@@H]2C[C@@H](O)CN2C(=O)[C@@H](NC(=O)[C@H]2CC3(CC[C@H](N4CCN(c5ccnc(N6CCc7[nH]c8nnc(-c9ccccc9O)cc8c7[C@H]6C)n5)CC4)CC3)C2)C(C)(C)C)cc1] |
| I-81 | 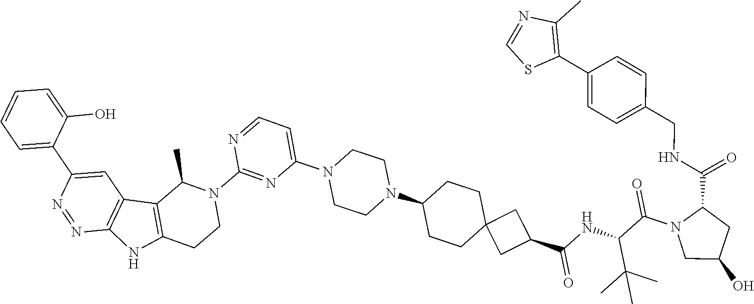 [SMILES: Cc1ncsc1-c1ccc(CNC(=O)[C@@H]2C[C@@H](O)CN2C(=O)[C@@H](NC(=O)[C@H]2CC3(CC[C@H](N4CCN(c5ccnc(N6CCc7[nH]c8nnc(-c9ccccc9O)cc8c7[C@H]6C)n5)CC4)CC3)C2)C(C)(C)C)cc1] |
| I-82 | 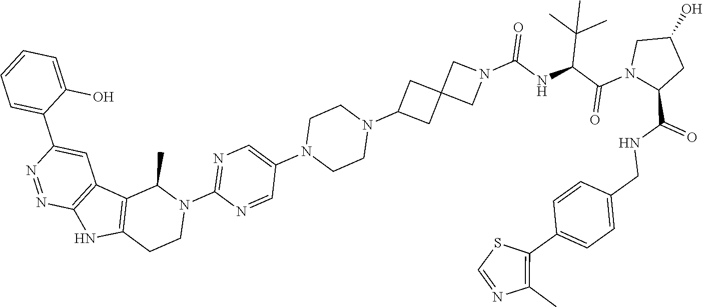 [SMILES: Cc1ncsc1-c1ccc(CNC(=O)[C@@H]2C[C@@H](O)CN2C(=O)[C@@H](NC(=O)N2CC3(CC(N4CCN(c5cnc(N6CCc7[nH]c8nnc(-c9ccccc9O)cc8c7[C@H]6C)nc5)CC4)C3)C2)C(C)(C)C)cc1] |
| I-83 | 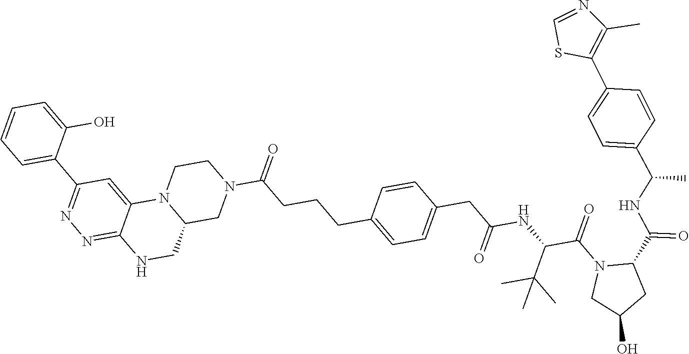 [SMILES: Cc1ncsc1-c1ccc([C@H](C)NC(=O)[C@@H]2C[C@@H](O)CN2C(=O)[C@@H](NC(=O)Cc2ccc(CCCC(=O)N3CCN4c5cc(-c6ccccc6O)nnc5NC[C@H]4C3)cc2)C(C)(C)C)cc1] |
| I-85 | 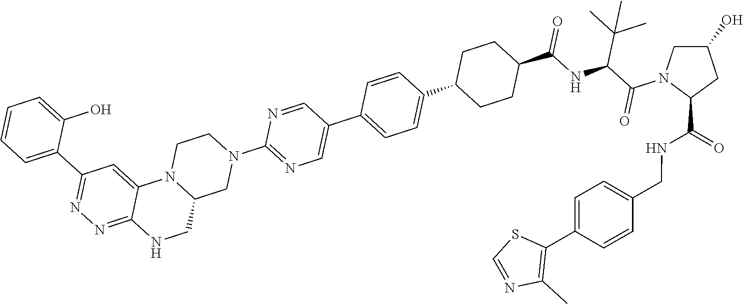 [SMILES: Cc1ncsc1-c1ccc(CNC(=O)[C@@H]2C[C@@H](O)CN2C(=O)[C@@H](NC(=O)[C@H]2CC[C@H](c3ccc(-c4cnc(N5CCN6c7cc(-c8ccccc8O)nnc7NC[C@H]6C5)nc4)cc3)CC2)C(C)(C)C)cc1] |
| I-86 | 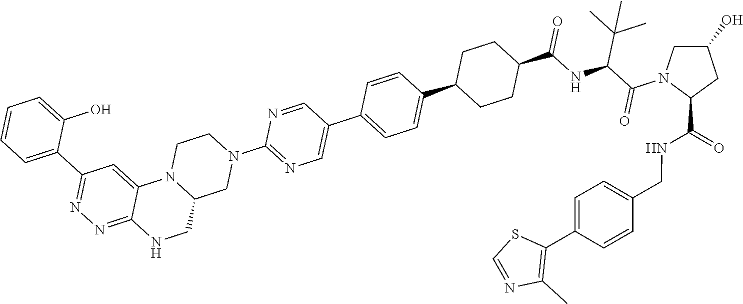 [SMILES: Cc1ncsc1-c1ccc(CNC(=O)[C@@H]2C[C@@H](O)CN2C(=O)[C@@H](NC(=O)[C@H]2CC[C@@H](c3ccc(-c4cnc(N5CCN6c7cc(-c8ccccc8O)nnc7NC[C@H]6C5)nc4)cc3)CC2)C(C)(C)C)cc1] |
| I-87 | 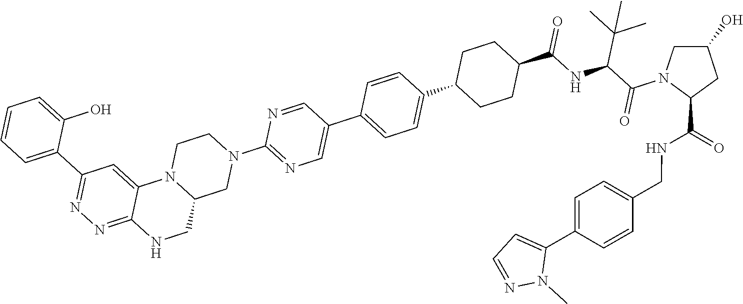 [SMILES: Cn1nccc1-c1ccc(CNC(=O)[C@@H]2C[C@@H](O)CN2C(=O)[C@@H](NC(=O)[C@H]2CC[C@H](c3ccc(-c4cnc(N5CCN6c7cc(-c8ccccc8O)nnc7NC[C@H]6C5)nc4)cc3)CC2)C(C)(C)C)cc1] |
| I-88 | 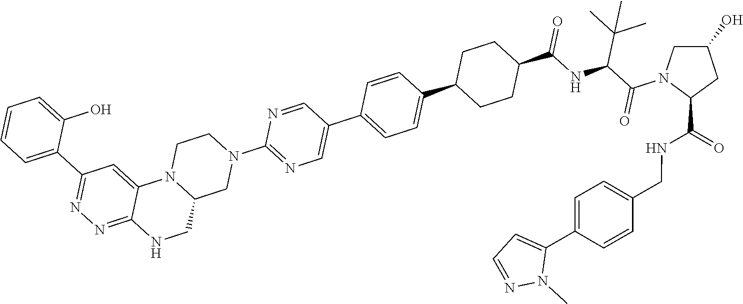 [SMILES: Cn1nccc1-c1ccc(CNC(=O)[C@@H]2C[C@@H](O)CN2C(=O)[C@@H](NC(=O)[C@H]2CC[C@@H](c3ccc(-c4cnc(N5CCN6c7cc(-c8ccccc8O)nnc7NC[C@H]6C5)nc4)cc3)CC2)C(C)(C)C)cc1] |
| I-89 | 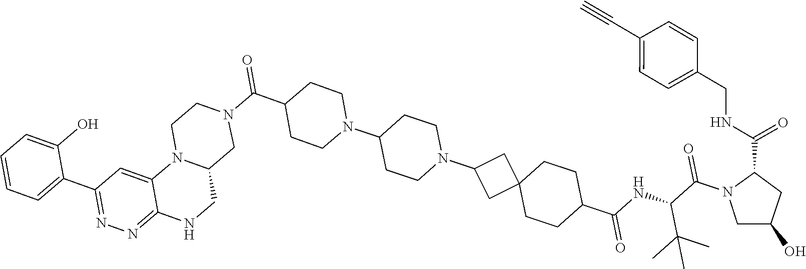 [SMILES: C#Cc1ccc(CNC(=O)[C@@H]2C[C@@H](O)CN2C(=O)[C@@H](NC(=O)C2CCC3(CC2)CC(N2CCC(N4CCC(C(=O)N5CCN6c7cc(-c8ccccc8O)nnc7NC[C@H]6C5)CC4)CC2)C3)C(C)(C)C)cc1] |
| I-90 | 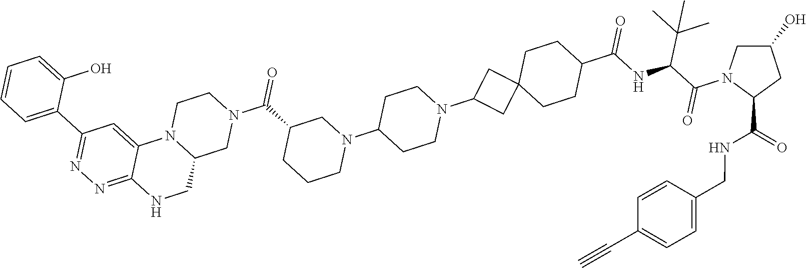 [SMILES: C#Cc1ccc(CNC(=O)[C@@H]2C[C@@H](O)CN2C(=O)[C@@H](NC(=O)C2CCC3(CC2)CC(N2CCC(N4CCC[C@H](C(=O)N5CCN6c7cc(-c8ccccc8O)nnc7NC[C@H]6C5)C4)CC2)C3)C(C)(C)C)cc1] |
| I-91 | 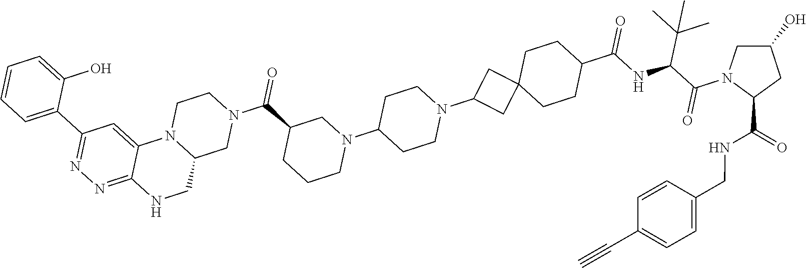 [SMILES: C#Cc1ccc(CNC(=O)[C@@H]2C[C@@H](O)CN2C(=O)[C@@H](NC(=O)C2CCC3(CC2)CC(N2CCC(N4CCC[C@@H](C(=O)N5CCN6c7cc(-c8ccccc8O)nnc7NC[C@H]6C5)C4)CC2)C3)C(C)(C)C)cc1] |
| I-92 | 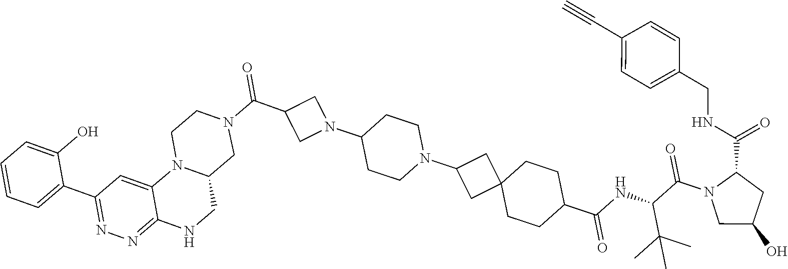 [SMILES: C#Cc1ccc(CNC(=O)[C@@H]2C[C@@H](O)CN2C(=O)[C@@H](NC(=O)C2CCC3(CC2)CC(N2CCC(N4CC(C(=O)N5CCN6c7cc(-c8ccccc8O)nnc7NC[C@H]6C5)C4)CC2)C3)C(C)(C)C)cc1] |
| I-93 | 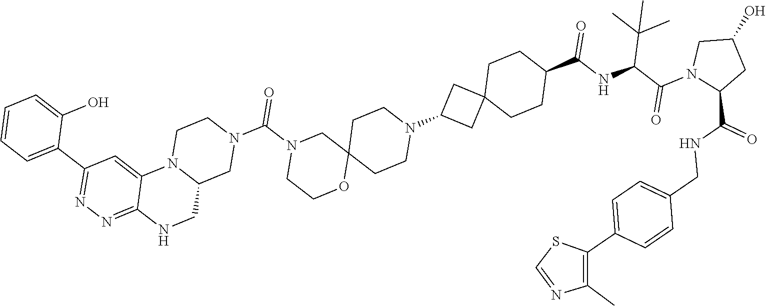 [SMILES: Cc1ncsc1-c1ccc(CNC(=O)[C@@H]2C[C@@H](O)CN2C(=O)[C@@H](NC(=O)[C@H]2CCC3(CC2)C[C@H](N2CCC4(CC2)CN(C(=O)N2CCN5c6cc(-c7ccccc7O)nnc6NC[C@H]5C2)CCO4)C3)C(C)(C)C)cc1] |
| I-94 | 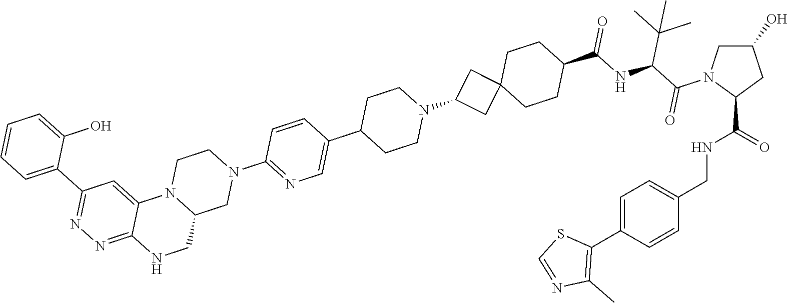 [SMILES: Cc1ncsc1-c1ccc(CNC(=O)[C@@H]2C[C@@H](O)CN2C(=O)[C@@H](NC(=O)[C@H]2CCC3(CC2)C[C@H](N2CCC(c4ccc(N5CCN6c7cc(-c8ccccc8O)nnc7NC[C@H]6C5)nc4)CC2)C3)C(C)(C)C)cc1] |
| I-95 | 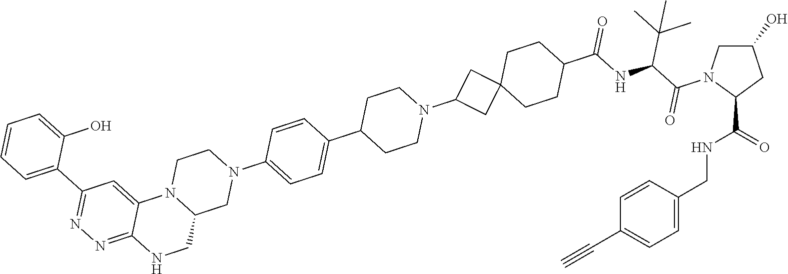 [SMILES: C#Cc1ccc(CNC(=O)[C@@H]2C[C@@H](O)CN2C(=O)[C@@H](NC(=O)C2CCC3(CC2)CC(N2CCC(c4ccc(N5CCN6c7cc(-c8ccccc8O)nnc7NC[C@H]6C5)cc4)CC2)C3)C(C)(C)C)cc1] |
| I-96 | 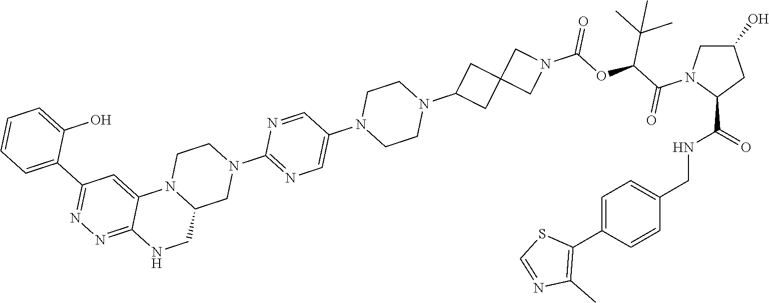 [SMILES: Cc1ncsc1-c1ccc(CNC(=O)[C@@H]2C[C@@H](O)CN2C(=O)[C@@H](OC(=O)N2CC3(CC(N4CCN(c5cnc(N6CCN7c8cc(-c9ccccc9O)nnc8NC[C@H]7C6)nc5)CC4)C3)C2)C(C)(C)C)cc1] |
| I-97 | 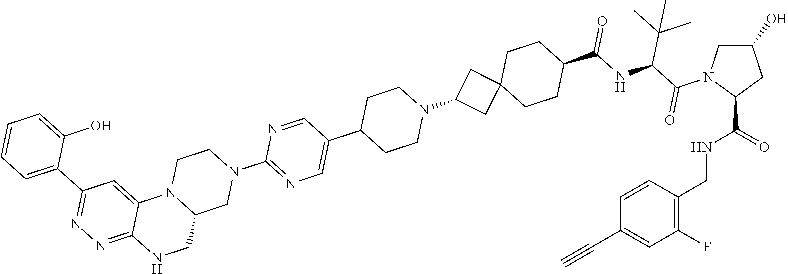 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(F)c1] |
| I-98 | 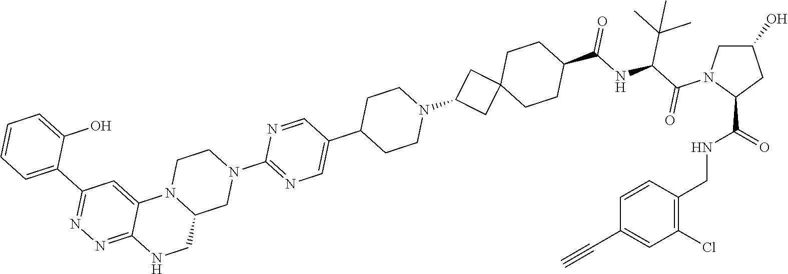 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(Cl)c1] |
| I-99 | 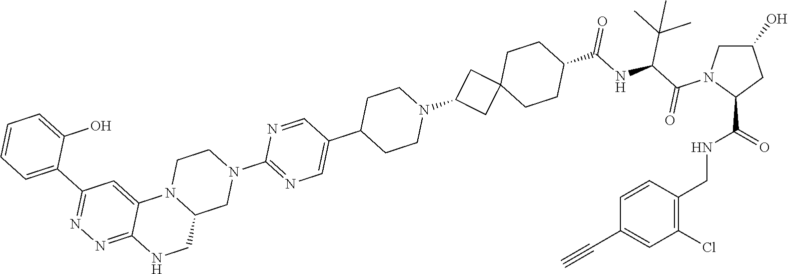 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(Cl)c1] |
| I-100 | 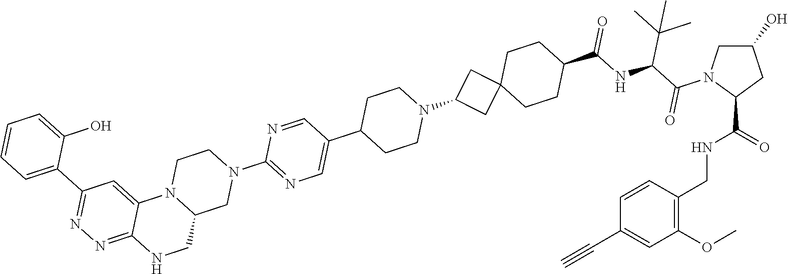 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(OC)c1] |
| I-101 | 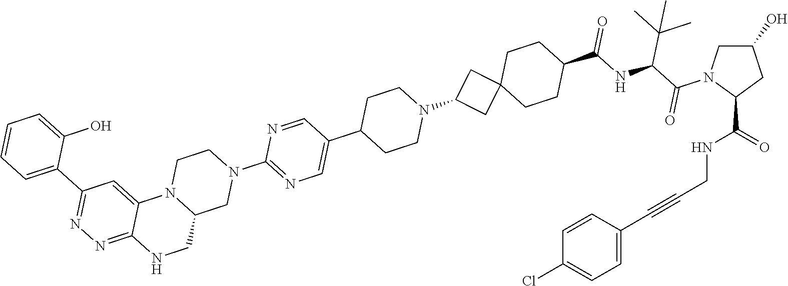 [SMILES: CC(C)(C)[C@H](NC(=O)[C@H]1CCC2(CC1)C[C@H](N1CCC(c3cnc(N4CCN5c6cc(-c7ccccc7O)nnc6NC[C@H]5C4)nc3)CC1)C2)C(=O)N1C[C@H](O)C[C@H]1C(=O)NCC#Cc1ccc(Cl)cc1] |
| I-102 | 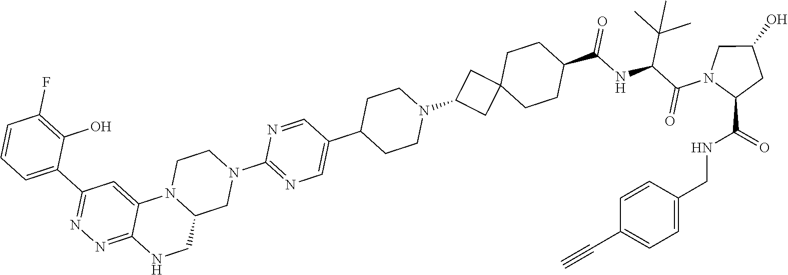 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8cccc(F)c8O)nnc7NC[C@H]6C5)nc4)CC2)C3)C(C)(C)C)cc1] |
| I-103 | 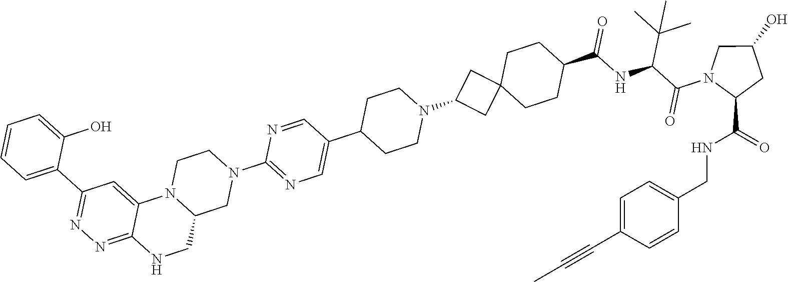 [SMILES: CC#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)cc1] |
| I-104 | 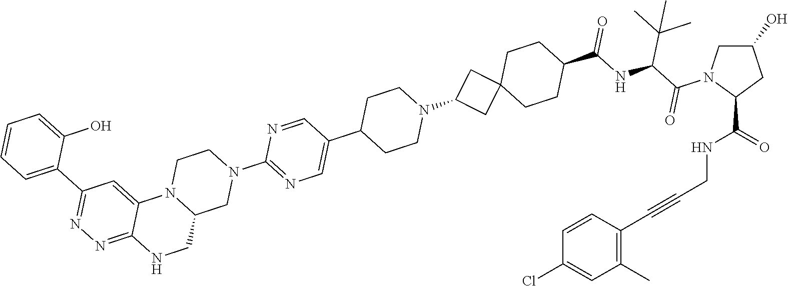 [SMILES: Cc1cc(Cl)ccc1C#CCNC(=O)[C@@H]1C[C@@H](O)CN1C(=O)[C@@H](NC(=O)[C@H]1CCC2(CC1)C[C@H](N1CCC(c3cnc(N4CCN5c6cc(-c7ccccc7O)nnc6NC[C@H]5C4)nc3)CC1)C2)C(C)(C)C] |
| I-105 | 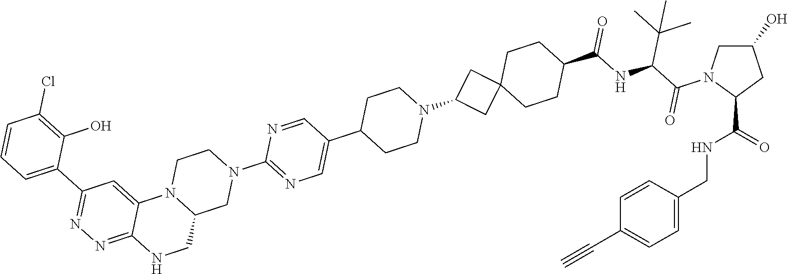 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8cccc(Cl)c8O)nnc7NC[C@H]6C5)nc4)CC2)C3)C(C)(C)C)cc1] |
| I-106 | 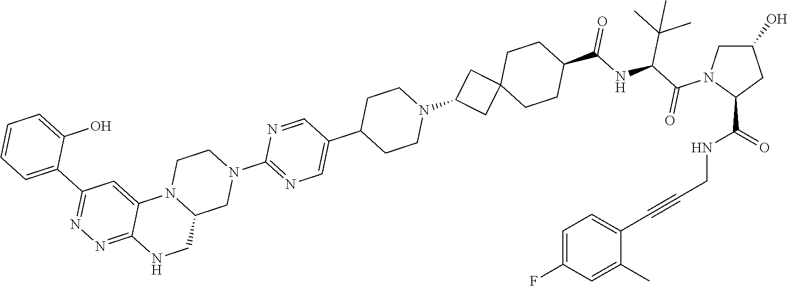 [SMILES: Cc1cc(F)ccc1C#CCNC(=O)[C@@H]1C[C@@H](O)CN1C(=O)[C@@H](NC(=O)[C@H]1CCC2(CC1)C[C@H](N1CCC(c3cnc(N4CCN5c6cc(-c7ccccc7O)nnc6NC[C@H]5C4)nc3)CC1)C2)C(C)(C)C] |
| I-107 | 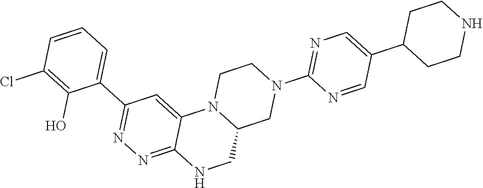 [SMILES: Oc1c(Cl)cccc1-c1cc2c(nn1)NC[C@H]1CN(c3ncc(C4CCNCC4)cn3)CCN21] |
| I-108 | 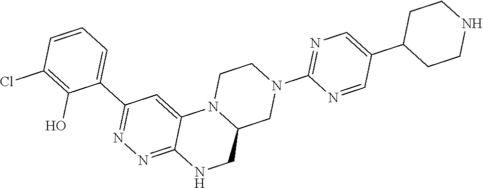 [SMILES: Oc1c(Cl)cccc1-c1cc2c(nn1)NC[C@@H]1CN(c3ncc(C4CCNCC4)cn3)CCN21] |
| I-111 | 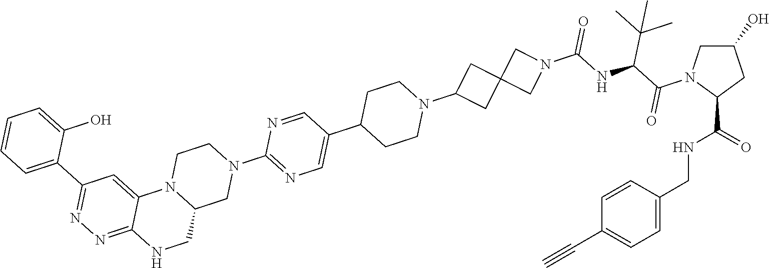 [SMILES: C#Cc1ccc(CNC(=O)[C@@H]2C[C@@H](O)CN2C(=O)[C@@H](NC(=O)N2CC3(CC(N4CCC(c5cnc(N6CCN7c8cc(-c9ccccc9O)nnc8NC[C@H]7C6)nc5)CC4)C3)C2)C(C)(C)C)cc1] |
| I-112 | 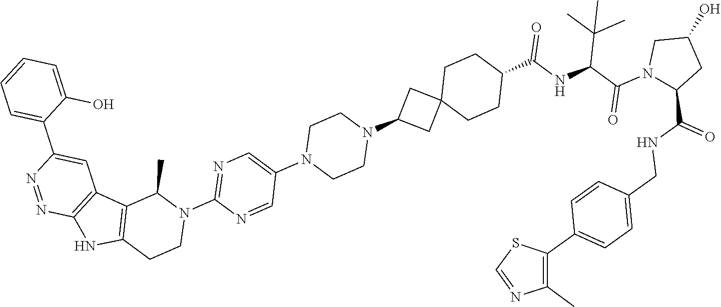 [SMILES: Cc1ncsc1-c1ccc(CNC(=O)[C@@H]2C[C@@H](O)CN2C(=O)[C@@H](NC(=O)[C@H]2CCC3(CC2)C[C@H](N2CCN(c4cnc(N5CCc6[nH]c7nnc(-c8ccccc8O)cc7c6[C@H]5C)nc4)CC2)C3)C(C)(C)C)cc1] |
| I-113 | 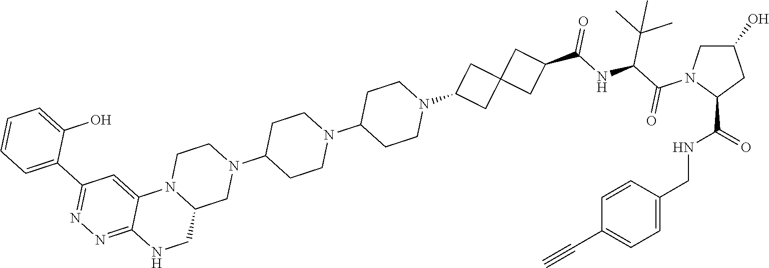 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)cc1] |
| I-114 | 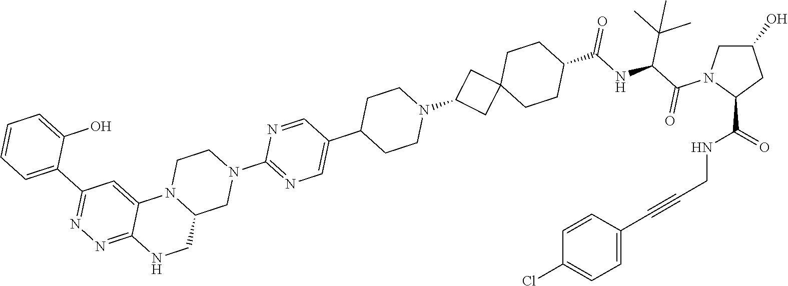 [SMILES: CC(C)(C)[C@H](NC(=O)[C@H]1CCC2(CC1)C[C@H](N1CCC(c3cnc(N4CCN5c6cc(-c7ccccc7O)nnc6NC[C@H]5C4)nc3)CC1)C2)C(=O)N1C[C@H](O)C[C@H]1C(=O)NCC#Cc1ccc(Cl)cc1] |
| I-115 | 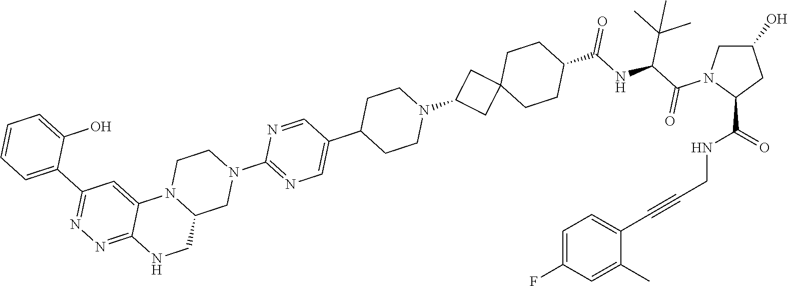 [SMILES: Cc1cc(F)ccc1C#CCNC(=O)[C@@H]1C[C@@H](O)CN1C(=O)[C@@H](NC(=O)[C@H]1CCC2(CC1)C[C@H](N1CCC(c3cnc(N4CCN5c6cc(-c7ccccc7O)nnc6NC[C@H]5C4)nc3)CC1)C2)C(C)(C)C] |
| I-116 | 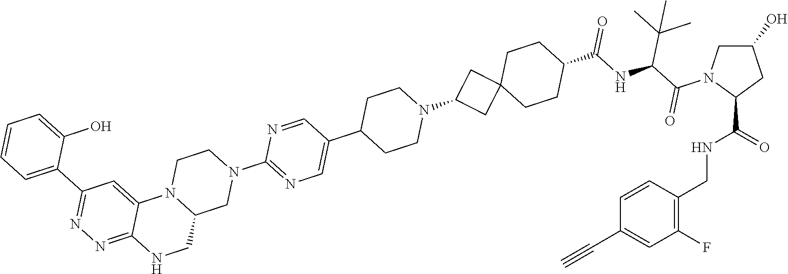 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(F)c1] |
| I-117 | 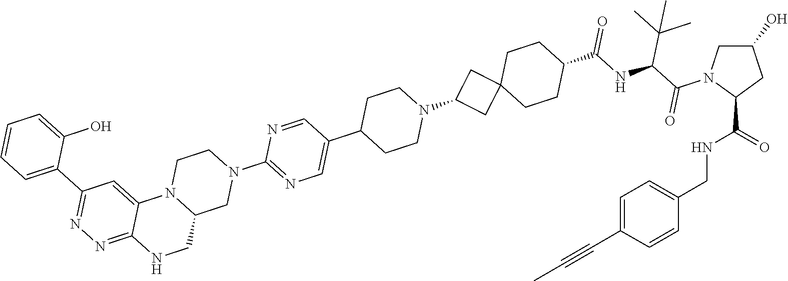 [SMILES: CC#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)cc1] |
| I-118 | 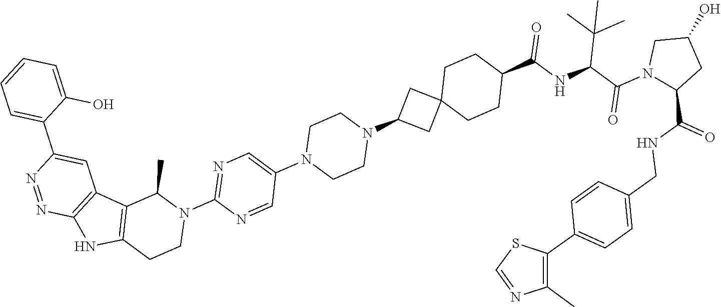 [SMILES: Cc1ncsc1-c1ccc(CNC(=O)[C@@H]2C[C@@H](O)CN2C(=O)[C@@H](NC(=O)[C@H]2CCC3(CC2)C[C@H](N2CCN(c4cnc(N5CCc6[nH]c7nnc(-c8ccccc8O)cc7c6[C@H]5C)nc4)CC2)C3)C(C)(C)C)cc1] |
| I-119 | 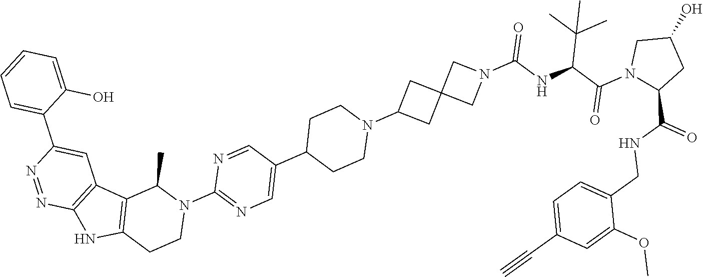 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(OC)c1] |
| I-120 | 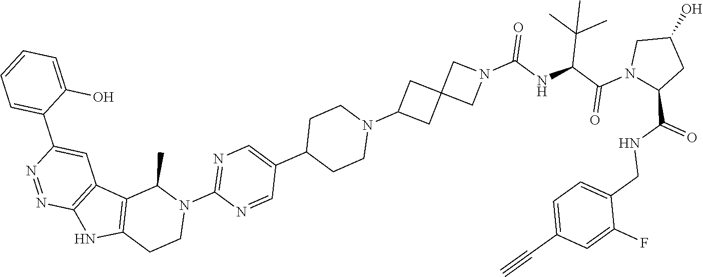 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(F)c1] |
| I-121 | 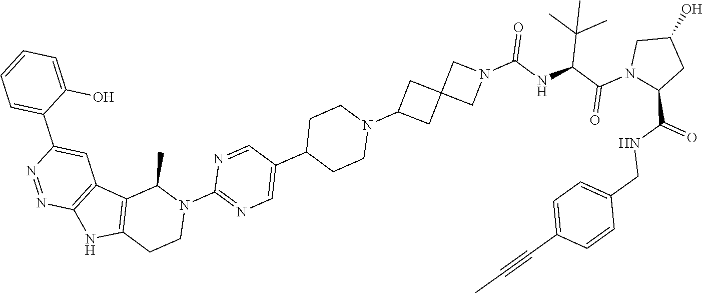 [SMILES: CC#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-122 | 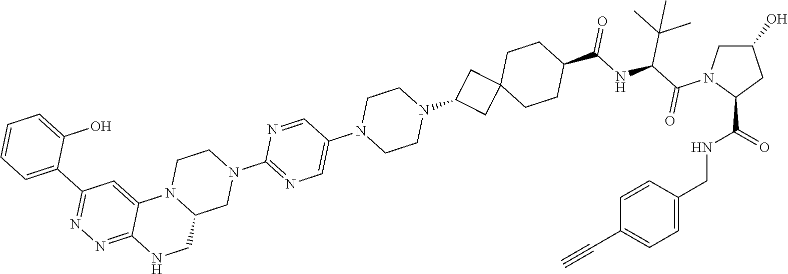 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)C)cc1] |
| I-125 | 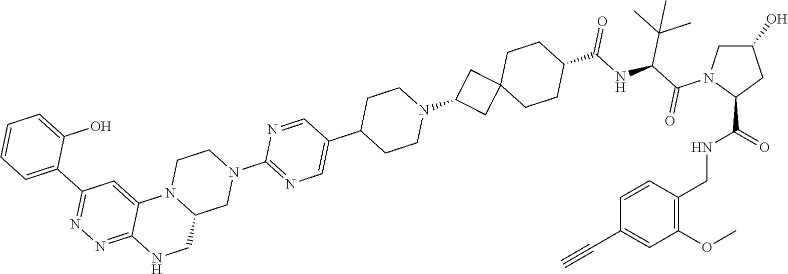 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(OC)c1] |
| I-126 | 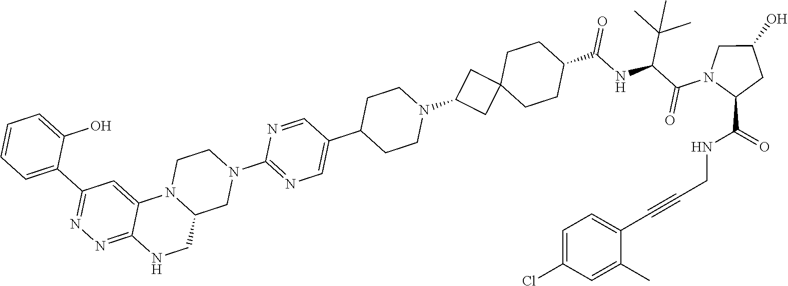 [SMILES: Cc1cc(Cl)ccc1C#CCNC(=O)[C@@H]1C[C@@H](O)CN1C(=O)[C@@H](NC(=O)[C@H]1CCC2(CC1)C[C@H](N1CCC(c3cnc(N4CCN5c6cc(-c7ccccc7O)nnc6NC[C@H]5C4)nc3)CC1)C2)C(C)(C)C] |
| I-127 | 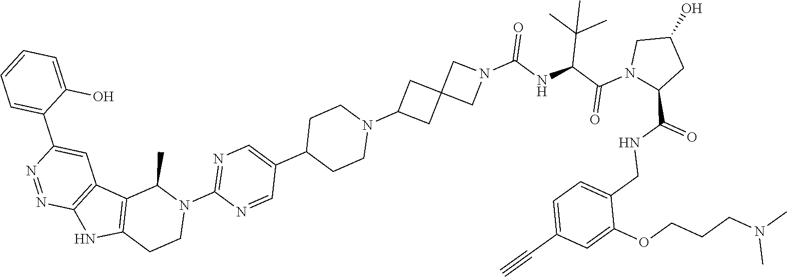 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(OCCCN(C)C)c1] |
| I-128 | 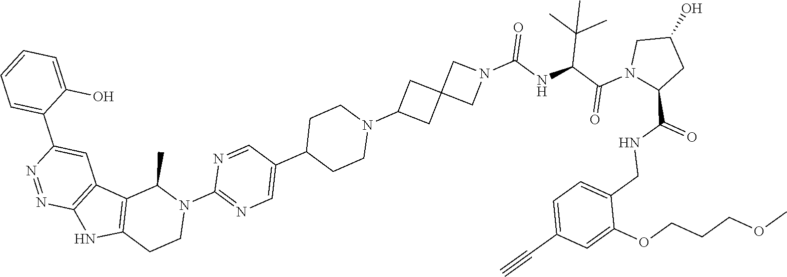 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(OCCCOC)c1] |
| I-129 | 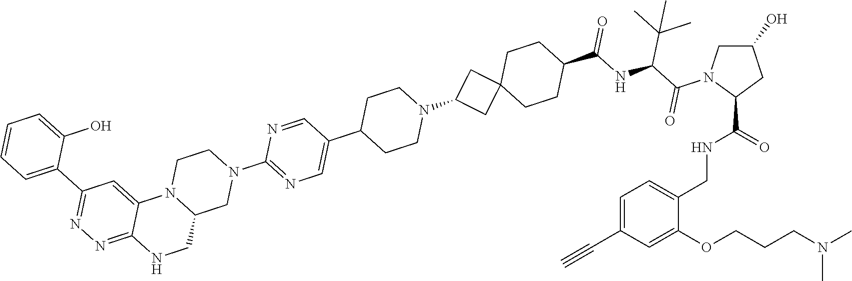 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(OCCCN(C)C)c1] |
| I-130 | 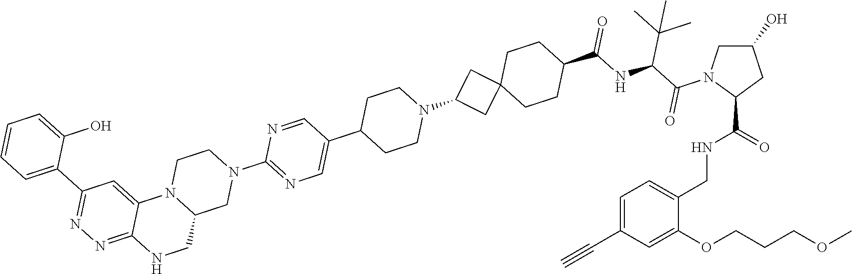 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(OCCCOC)c1] |
| I-131 | 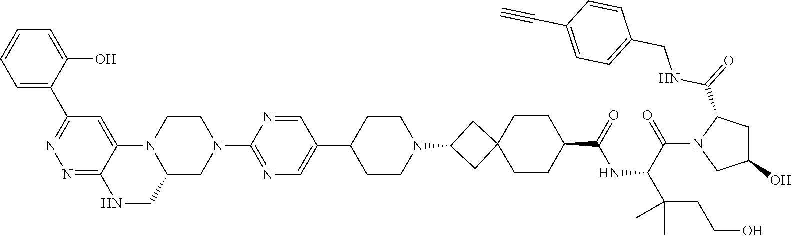 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)CCO)cc1] |
| I-132 | 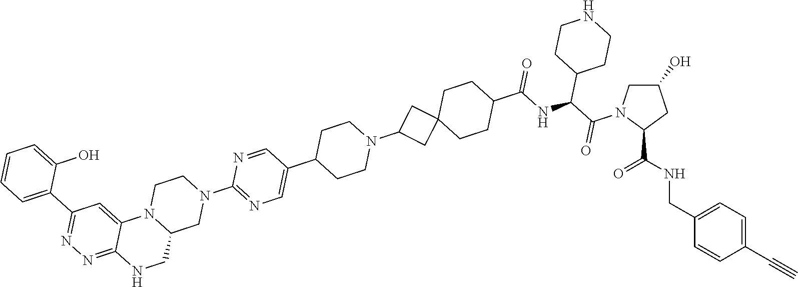 [SMILES: C#Cc1ccc(CNC(=O)[C@@H]2C[C@@H](O)CN2C(=O)[C@@H](NC(=O)C2CCC3(CC2)CC(N2CCC(c4cnc(N5CCN6c7cc(-c8ccccc8O)nnc7NC[C@H]6C5)nc4)CC2)C3)C2CCNCC2)cc1] |
| I-133 | 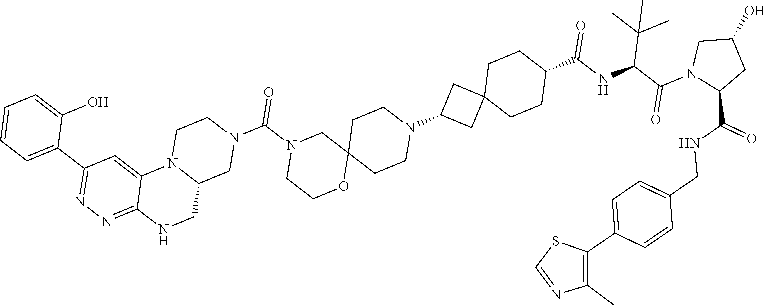 [SMILES: Cc1ncsc1-c1ccc(CNC(=O)[C@@H]2C[C@@H](O)CN2C(=O)[C@@H](NC(=O)[C@H]2CCC3(CC2)C[C@H](N2CCC4(CC2)CN(C(=O)N2CCN5c6cc(-c7ccccc7O)nnc6NC[C@H]5C2)CCO4)C3)C(C)(C)C)cc1] |
| I-134 | 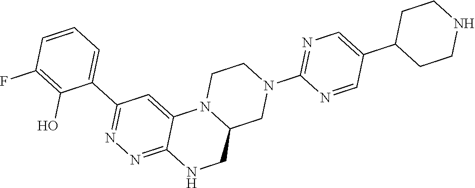 [SMILES: Oc1c(F)cccc1-c1cc2c(nn1)NC[C@@H]1CN(c3ncc(C4CCNCC4)cn3)CCN21] |
| I-135 | 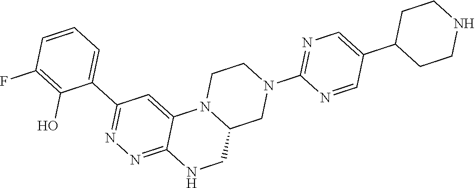 [SMILES: Oc1c(F)cccc1-c1cc2c(nn1)NC[C@H]1CN(c3ncc(C4CCNCC4)cn3)CCN21] |
| I-136 | 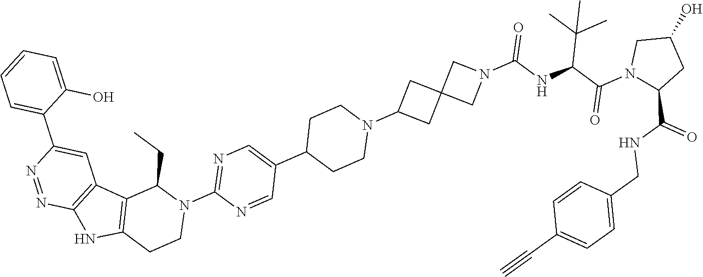 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6CC)nc5)CC4)C3)C2)C(C)(C)C)cc1] |
| I-137 | 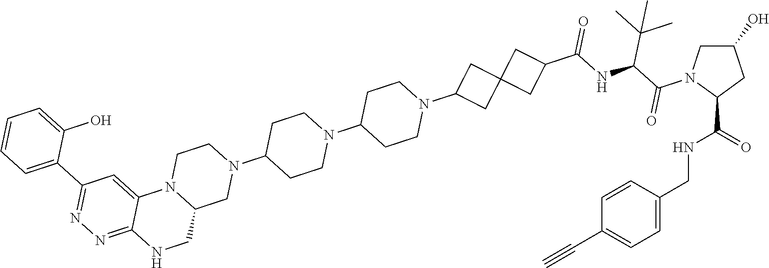 [SMILES: C#Cc1ccc(CNC(=O)[C@@H]2C[C@@H](O)CN2C(=O)[C@@H](NC(=O)C2CC3(C2)CC(N2CCC(N4CCC(N5CCN6c7cc(-c8ccccc8O)nnc7NC[C@H]6C5)CC4)CC2)C3)C(C)(C)C)cc1] |
| I-138 | 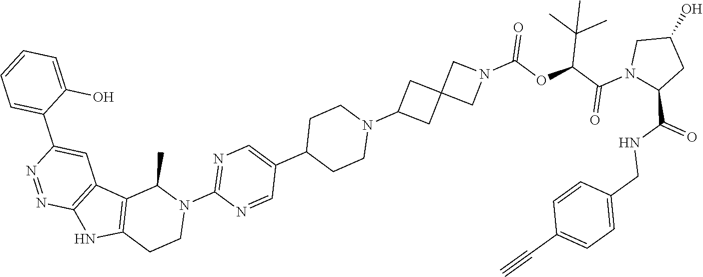 [SMILES: C#Cc1ccc(CNC(=O)[C@@H]2C[C@@H](O)CN2C(=O)[C@@H](OC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-139 | 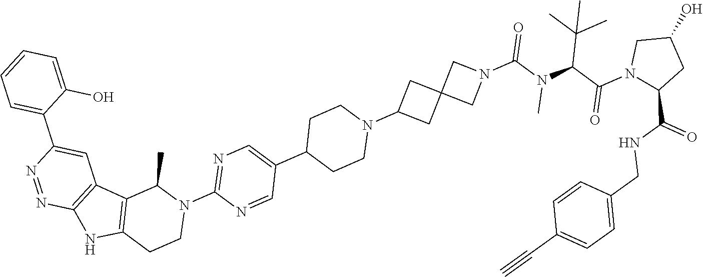 [SMILES: C#Cc1ccc(CNC(=O)[C@@H]2C[C@@H](O)CN2C(=O)[C@@H](N(C)C(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-140 | 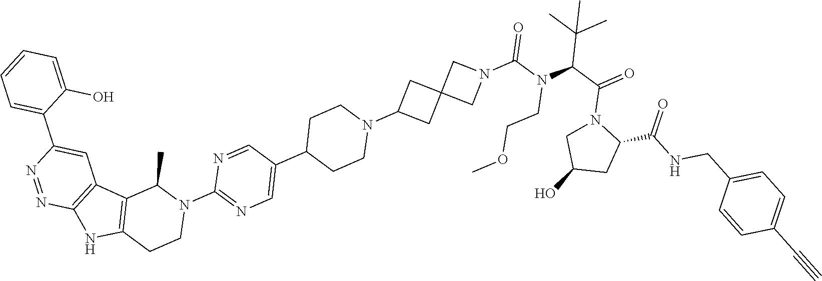 [SMILES: C#Cc1ccc(CNC(=O)[C@@H]2C[C@@H](O)CN2C(=O)[C@@H](N(CCOC)C(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-141 | 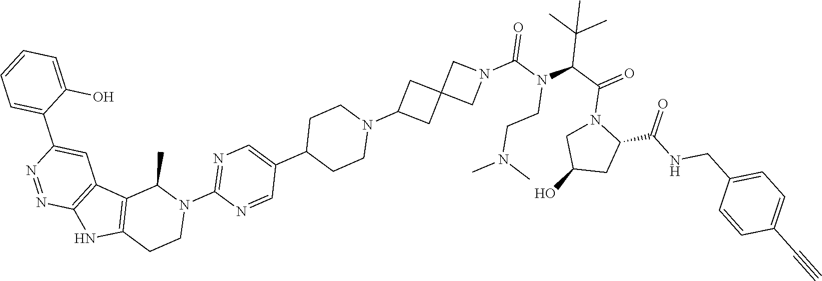 [SMILES: C#Cc1ccc(CNC(=O)[C@@H]2C[C@@H](O)CN2C(=O)[C@@H](N(CCN(C)C)C(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-142 | 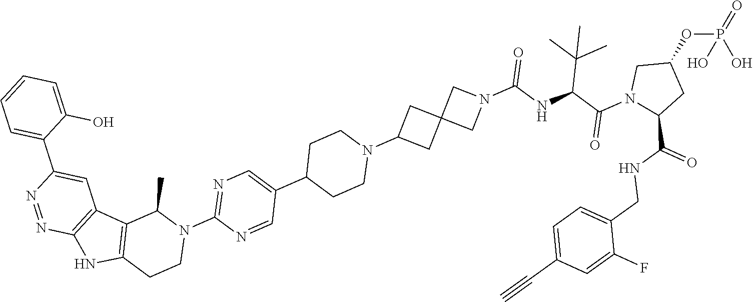 [SMILES: C#Cc1ccc(CNC(=O)[C@@H]2C[C@@H](OP(=O)(O)O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(F)c1] |
| I-143 | 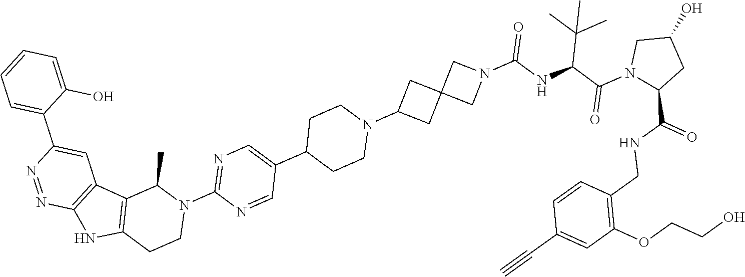 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(OCCO)c1] |
| I-144 | 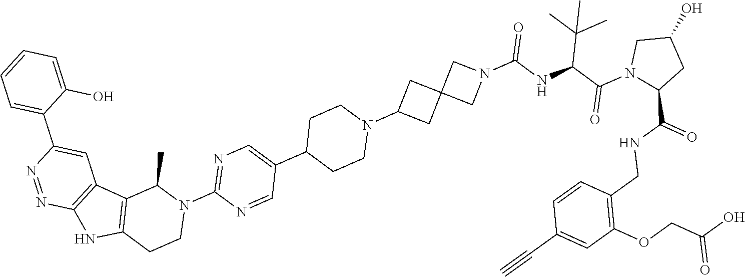 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(OCC(=O)O)c1] |
| I-145 | 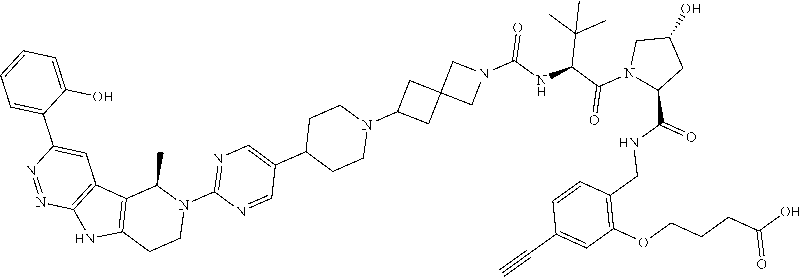 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(OCCCC(=O)O)c1] |
| I-146 | 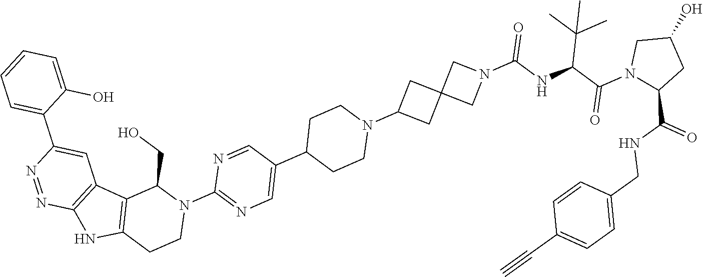 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6CO)nc5)CC4)C3)C2)C(C)(C)C)cc1] |
| I-147 | 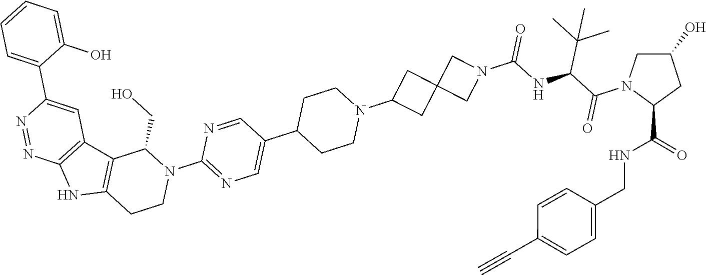 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@@H]6CO)nc5)CC4)C3)C2)C(C)(C)C)cc1] |
| I-148 | 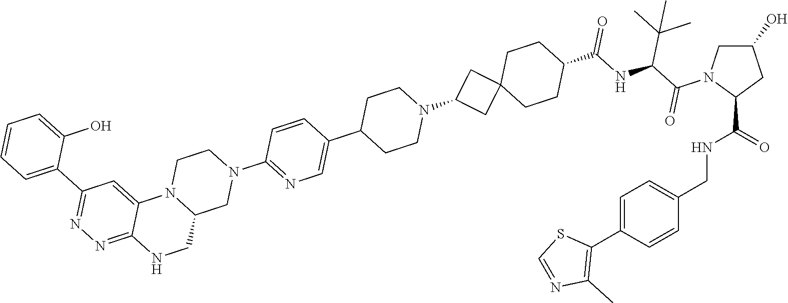 [SMILES: Cc1ncsc1-c1ccc(CNC(=O)[C@@H]2C[C@@H](O)CN2C(=O)[C@@H](NC(=O)[C@H]2CCC3(CC2)C[C@H](N2CCC(c4ccc(N5CCN6c7cc(-c8ccccc8O)nnc7NC[C@H]6C5)nc4)CC2)C3)C(C)(C)C)cc1] |
| I-149 | 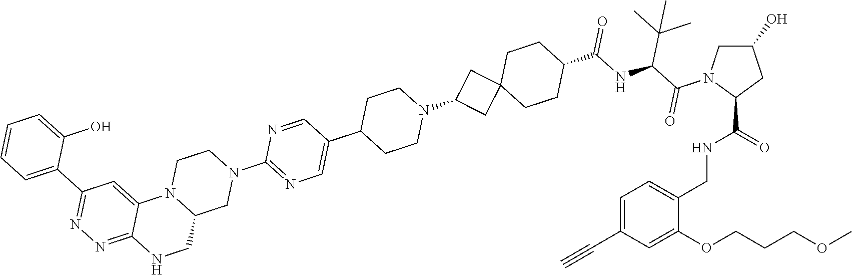 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(OCCCOC)c1] |
| I-150 | 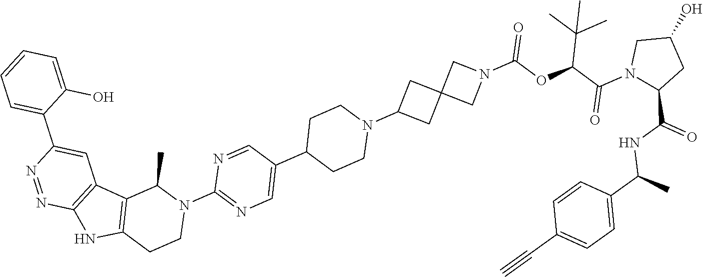 [SMILES: C#Cc1ccc([C@H](C)NC(=O)[C@@H]2C[C@@H](O)CN2C(=O)[C@@H](OC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-151 | 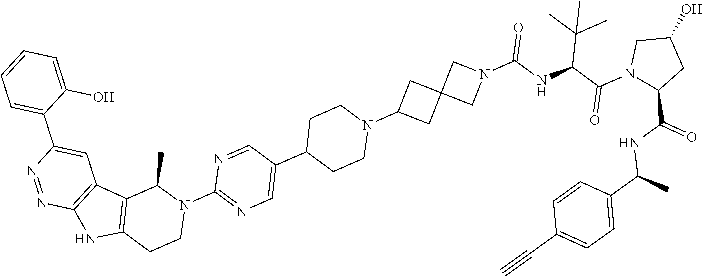 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-152 | 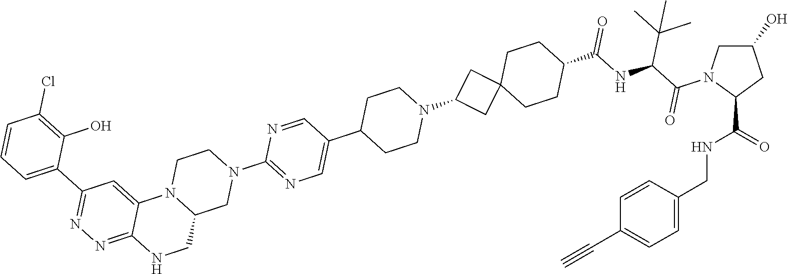 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8cccc(Cl)c8O)nnc7NC[C@H]6C5)nc4)CC2)C3)C(C)(C)C)cc1] |
| I-153 | 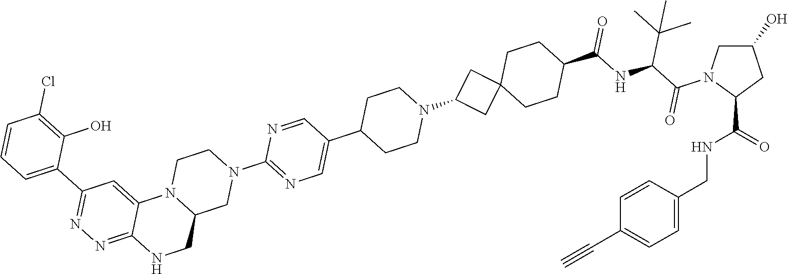 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8cccc(Cl)c8O)nnc7NC[C@@H]6C5)nc4)CC2)C3)C(C)(C)C)cc1] |
| I-154 | 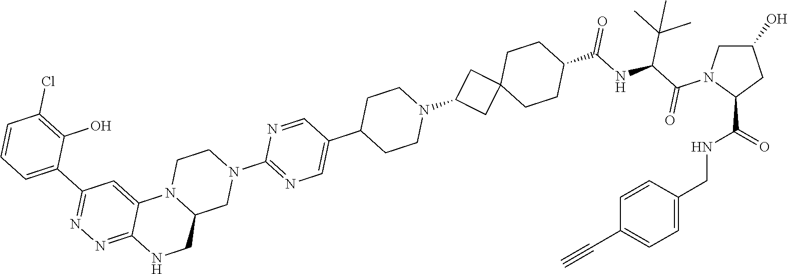 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8cccc(Cl)c8O)nnc7NC[C@@H]6C5)nc4)CC2)C3)C(C)(C)C)cc1] |
| I-155 | 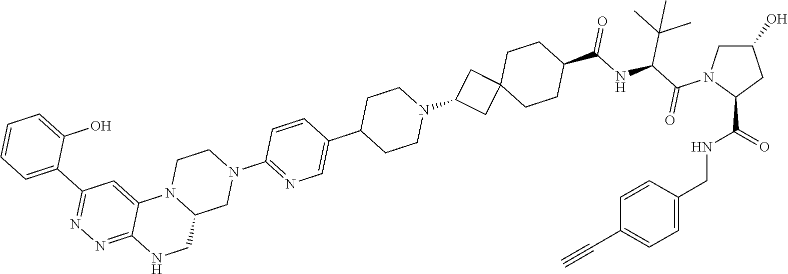 [SMILES: C#Cc1ccc(CNC(=O)[C@@H]2C[C@@H](O)CN2C(=O)[C@@H](NC(=O)[C@H]2CCC3(CC2)C[C@H](N2CCC(c4ccc(N5CCN6c7cc(-c8ccccc8O)nnc7NC[C@H]6C5)nc4)CC2)C3)C(C)(C)C)cc1] |
| I-156 | 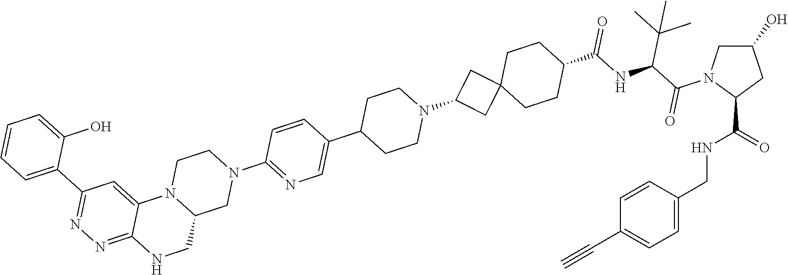 [SMILES: C#Cc1ccc(CNC(=O)[C@@H]2C[C@@H](O)CN2C(=O)[C@@H](NC(=O)[C@H]2CCC3(CC2)C[C@H](N2CCC(c4ccc(N5CCN6c7cc(-c8ccccc8O)nnc7NC[C@H]6C5)nc4)CC2)C3)C(C)(C)C)cc1] |
| I-157 | 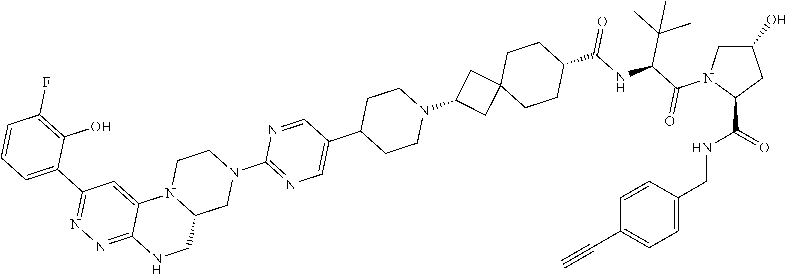 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8cccc(F)c8O)nnc7NC[C@H]6C5)nc4)CC2)C3)C(C)(C)C)cc1] |
| I-158 | 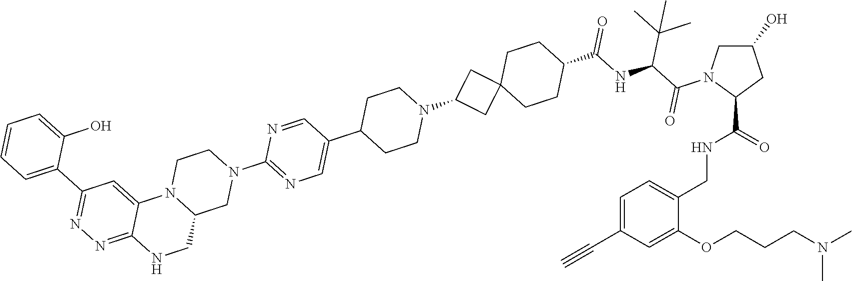 [SMILES: C#Cc1ccc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(OCCCN(C)C)c1] |
| I-159 | 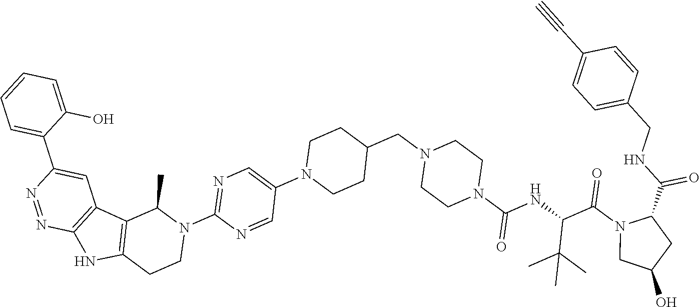 [SMILES: C#Cc1ccc(CNC(=O)[C@@H]2C[C@@H](O)CN2C(=O)[C@@H](NC(=O)N2CCN(CC3CCN(c4cnc(N5CCc6[nH]c7nnc(-c8ccccc8O)cc7c6[C@H]5C)nc4)CC3)CC2)C(C)(C)C)cc1] |
| I-160 | 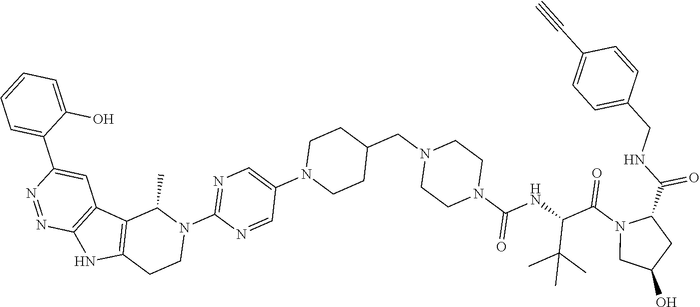 [SMILES: C#Cc1ccc(CNC(=O)[C@@H]2C[C@@H](O)CN2C(=O)[C@@H](NC(=O)N2CCN(CC3CCN(c4cnc(N5CCc6[nH]c7nnc(-c8ccccc8O)cc7c6[C@@H]5C)nc4)CC3)CC2)C(C)(C)C)cc1] |
| I-161 | 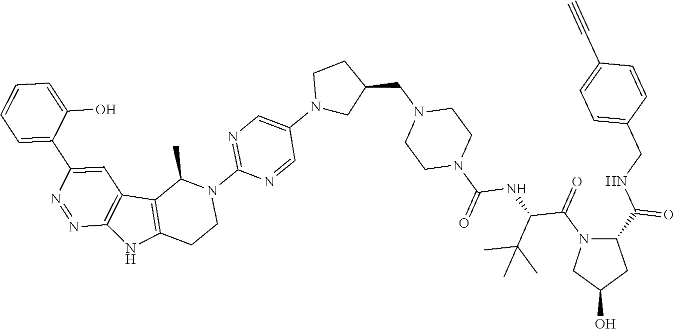 [SMILES: C#Cc1ccc(CNC(=O)[C@@H]2C[C@@H](O)CN2C(=O)[C@@H](NC(=O)N2CCN(C[C@@H]3CCN(c4cnc(N5CCc6[nH]c7nnc(-c8ccccc8O)cc7c6[C@H]5C)nc4)C3)CC2)C(C)(C)C)cc1] |
| I-162 | 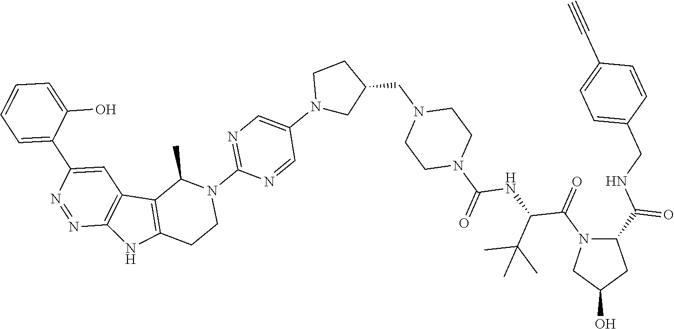 [SMILES: C#Cc1ccc(CNC(=O)[C@@H]2C[C@@H](O)CN2C(=O)[C@@H](NC(=O)N2CCN(C[C@H]3CCN(c4cnc(N5CCc6[nH]c7nnc(-c8ccccc8O)cc7c6[C@H]5C)nc4)C3)CC2)C(C)(C)C)cc1] |
| I-163 | 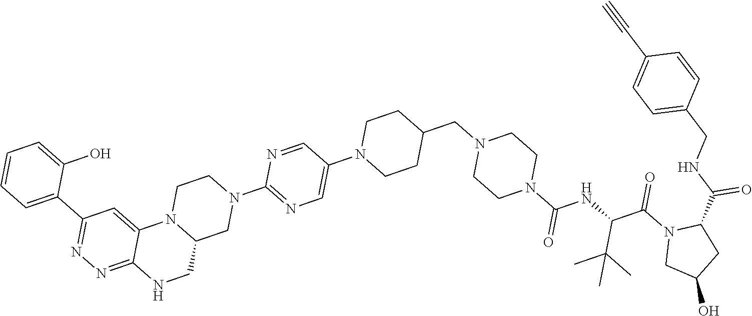 [SMILES: C#Cc1ccc(CNC(=O)[C@@H]2C[C@@H](O)CN2C(=O)[C@@H](NC(=O)N2CCN(CC3CCN(c4cnc(N5CCN6c7cc(-c8ccccc8O)nnc7NC[C@H]6C5)nc4)CC3)CC2)C(C)(C)C)cc1] |
| I-164 | 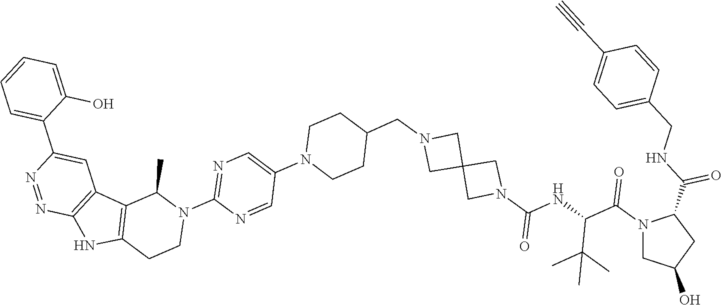 [SMILES: C#Cc1ccc(CNC(=O)[C@@H]2C[C@@H](O)CN2C(=O)[C@@H](NC(=O)N2CC3(CN(CC4CCN(c5cnc(N6CCc7[nH]c8nnc(-c9ccccc9O)cc8c7[C@H]6C)nc5)CC4)C3)C2)C(C)(C)C)cc1] |
| I-165 | 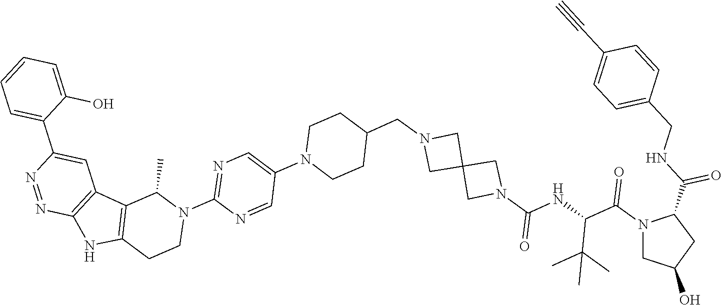 [SMILES: C#Cc1ccc(CNC(=O)[C@@H]2C[C@@H](O)CN2C(=O)[C@@H](NC(=O)N2CC3(CN(CC4CCN(c5cnc(N6CCc7[nH]c8nnc(-c9ccccc9O)cc8c7[C@@H]6C)nc5)CC4)C3)C2)C(C)(C)C)cc1] |
| I-166 | 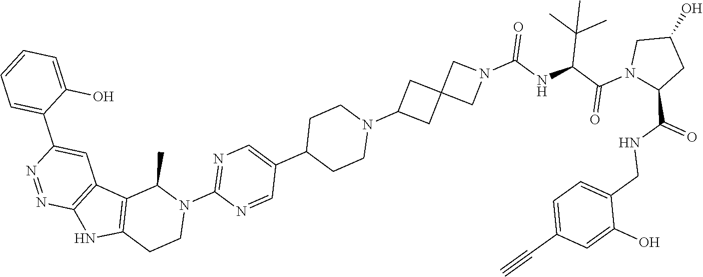 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(O)c1] |
| I-167 | 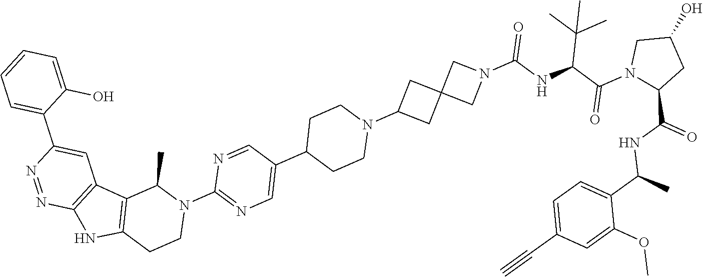 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(OC)c1] |
| I-168 | 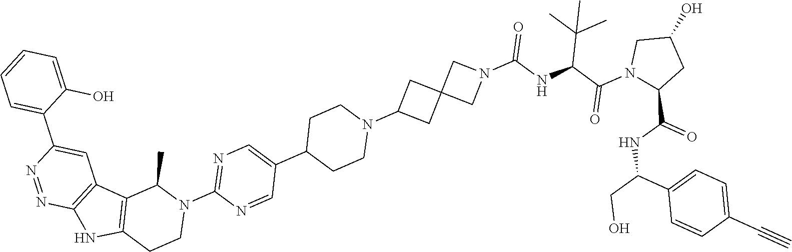 [SMILES: C#Cc1ccc([C@H](CO)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-169 | 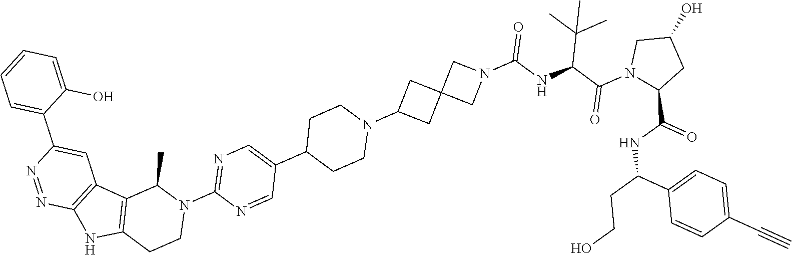 [SMILES: C#Cc1ccc([C@H](CCO)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1] |
| I-170 | 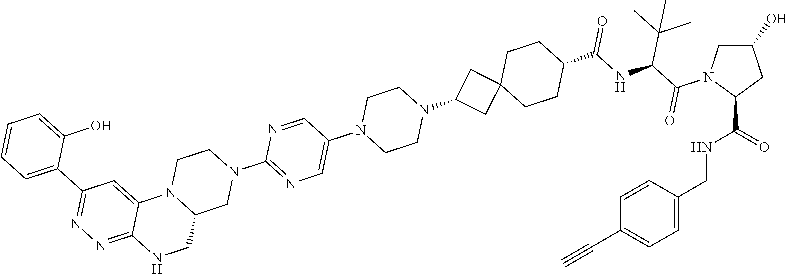 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)C)cc1] |
| I-171 | 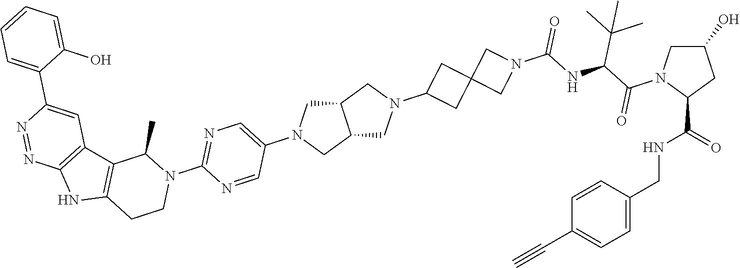 [SMILES: C#Cc1ccc(CNC(=O)[C@@H]2C[C@@H](O)CN2C(=O)[C@@H](NC(=O)N2CC3(CC(N4C[C@H]5CN(c6cnc(N7CCc8[nH]c9nnc(-c%10ccccc%10O)cc9c8[C@H]7C)nc6)C[C@H]5C4)C3)C2)C(C)(C)C)cc1] |
| I-172 | 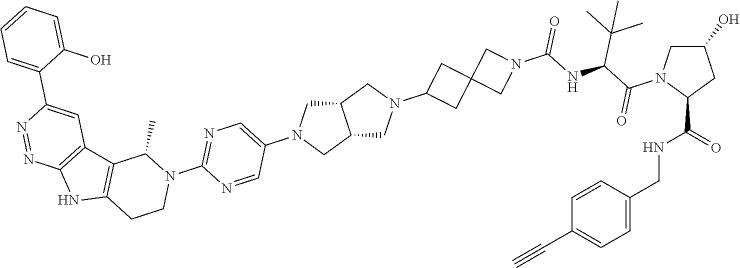 [SMILES: C#Cc1ccc(CNC(=O)[C@@H]2C[C@@H](O)CN2C(=O)[C@@H](NC(=O)N2CC3(CC(N4C[C@H]5CN(c6cnc(N7CCc8[nH]c9nnc(-c%10ccccc%10O)cc9c8[C@@H]7C)nc6)C[C@H]5C4)C3)C2)C(C)(C)C)cc1] |
| I-173 | 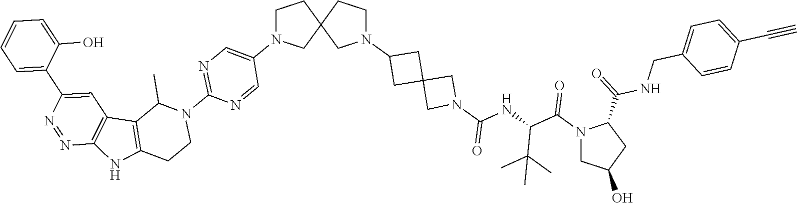 [SMILES: C#Cc1ccc(CNC(=O)[C@@H]2C[C@@H](O)CN2C(=O)[C@@H](NC(=O)N2CC3(CC(N4CCC5(CCN(c6cnc(N7CCc8[nH]c9nnc(-c%10ccccc%10O)cc9c8C7C)nc6)C5)C4)C3)C2)C(C)(C)C)cc1] |
| I-174 | 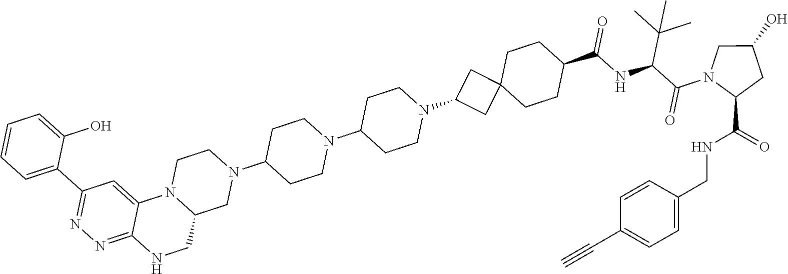 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CCC(N5CCN6c7cc(-c8ccccc8O)nnc7NC[C@H]6C5)CC4)CC2)C3)C(C)(C)C)cc1] |
| I-175 | 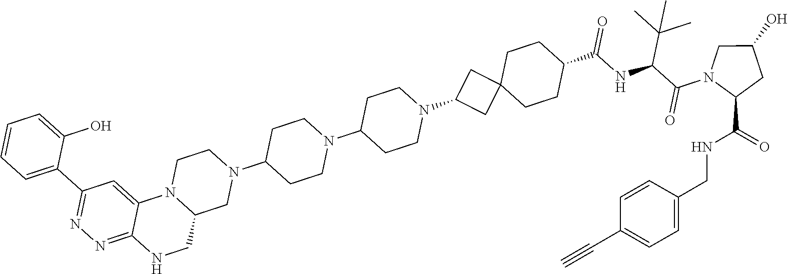 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CCC(N5CCN6c7cc(-c8ccccc8O)nnc7NC[C@H]6C5)CC4)CC2)C3)C(C)(C)C)cc1] |
| I-176 | 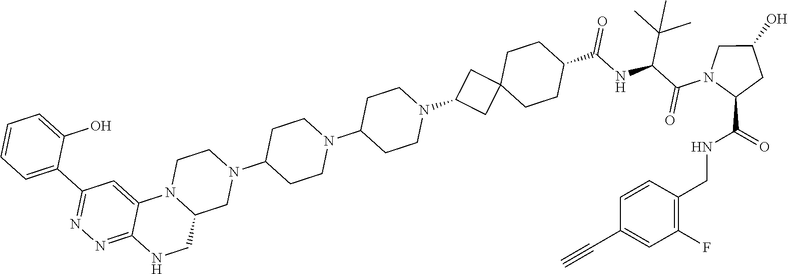 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CCC(N5CCN6c7cc(-c8ccccc8O)nnc7NC[C@H]6C5)CC4)CC2)C3)C(C)(C)C)c(F)c1] |
| I-177 | 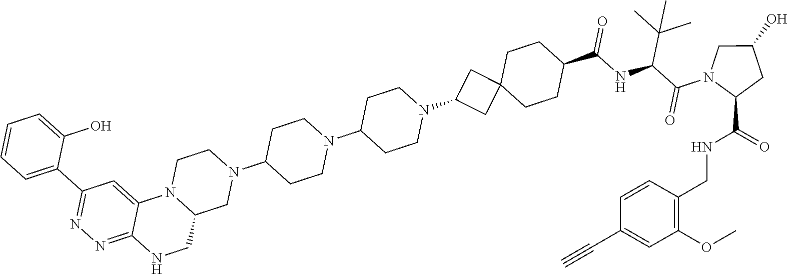 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CCC(N5CCN6c7cc(-c8ccccc8O)nnc7NC[C@H]6C5)CC4)CC2)C3)C(C)(C)C)c(OC)c1] |
| I-178 | 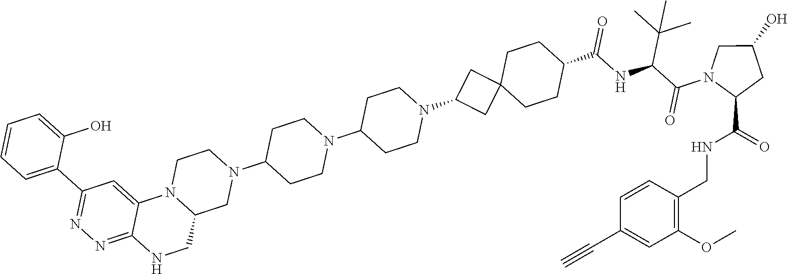 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CCC(N5CCN6c7cc(-c8ccccc8O)nnc7NC[C@H]6C5)CC4)CC2)C3)C(C)(C)C)c(OC)c1] |
| I-179 | 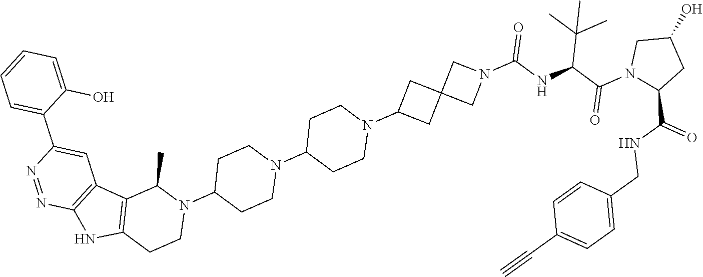 [SMILES: C#Cc1ccc(CNC(=O)[C@@H]2C[C@@H](O)CN2C(=O)[C@@H](NC(=O)N2CC3(CC(N4CCC(N5CCC(N6CCc7[nH]c8nnc(-c9ccccc9O)cc8c7[C@H]6C)CC5)CC4)C3)C2)C(C)(C)C)cc1] |
| I-180 | 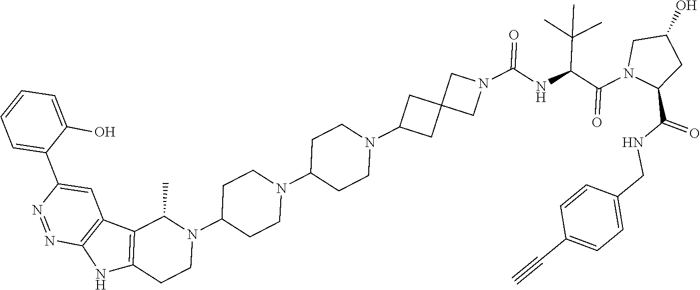 [SMILES: C#Cc1ccc(CNC(=O)[C@@H]2C[C@@H](O)CN2C(=O)[C@@H](NC(=O)N2CC3(CC(N4CCC(N5CCC(N6CCc7[nH]c8nnc(-c9ccccc9O)cc8c7[C@@H]6C)CC5)CC4)C3)C2)C(C)(C)C)cc1] |
| I-181 | 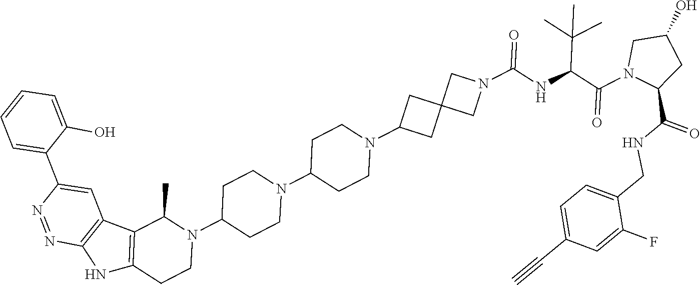 [SMILES: C#Cc1ccc(CNC(=O)[C@@H]2C[C@@H](O)CN2C(=O)[C@@H](NC(=O)N2CC3(CC(N4CCC(N5CCC(N6CCc7[nH]c8nnc(-c9ccccc9O)cc8c7[C@H]6C)CC5)CC4)C3)C2)C(C)(C)C)c(F)c1] |
| I-182 | 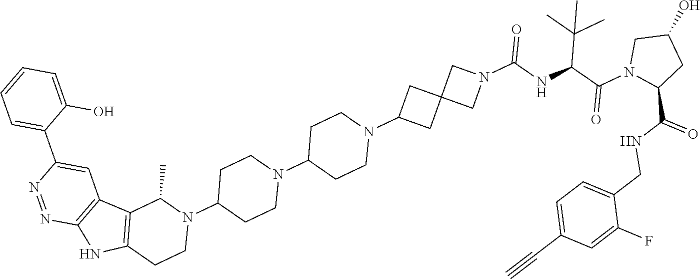 [SMILES: C#Cc1ccc(CNC(=O)[C@@H]2C[C@@H](O)CN2C(=O)[C@@H](NC(=O)N2CC3(CC(N4CCC(N5CCC(N6CCc7[nH]c8nnc(-c9ccccc9O)cc8c7[C@@H]6C)CC5)CC4)C3)C2)C(C)(C)C)c(F)c1] |
| I-183 | 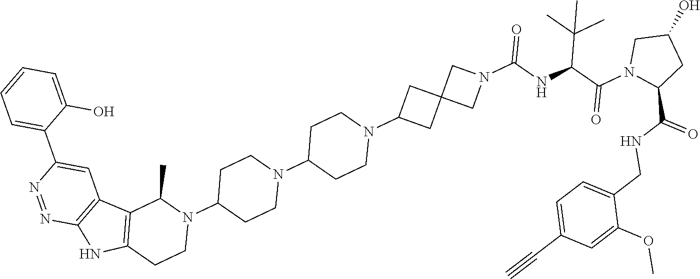 [SMILES: C#Cc1ccc(CNC(=O)[C@@H]2C[C@@H](O)CN2C(=O)[C@@H](NC(=O)N2CC3(CC(N4CCC(N5CCC(N6CCc7[nH]c8nnc(-c9ccccc9O)cc8c7[C@H]6C)CC5)CC4)C3)C2)C(C)(C)C)c(OC)c1] |
| I-184 | 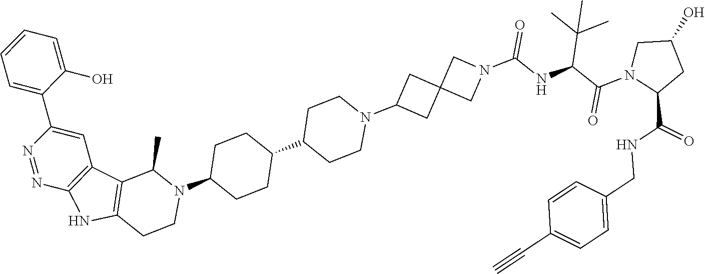 [SMILES: C#Cc1ccc(CNC(=O)[C@@H]2C[C@@H](O)CN2C(=O)[C@@H](NC(=O)N2CC3(CC(N4CCC([C@H]5CC[C@H](N6CCc7[nH]c8nnc(-c9ccccc9O)cc8c7[C@H]6C)CC5)CC4)C3)C2)C(C)(C)C)cc1] |
| I-185 | 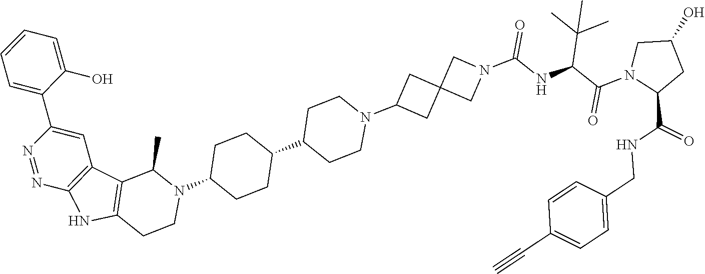 [SMILES: C#Cc1ccc(CNC(=O)[C@@H]2C[C@@H](O)CN2C(=O)[C@@H](NC(=O)N2CC3(CC(N4CCC([C@H]5CC[C@@H](N6CCc7[nH]c8nnc(-c9ccccc9O)cc8c7[C@H]6C)CC5)CC4)C3)C2)C(C)(C)C)cc1] |
| I-186 | 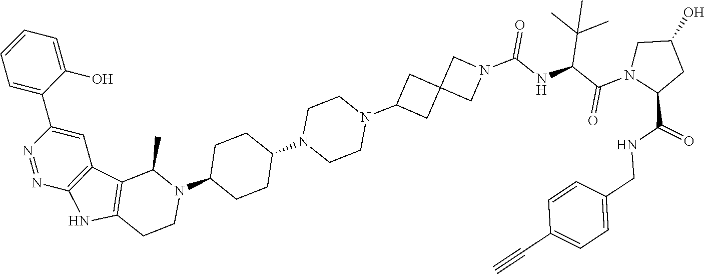 [SMILES: C#Cc1ccc(CNC(=O)[C@@H]2C[C@@H](O)CN2C(=O)[C@@H](NC(=O)N2CC3(CC(N4CCN([C@H]5CC[C@H](N6CCc7[nH]c8nnc(-c9ccccc9O)cc8c7[C@H]6C)CC5)CC4)C3)C2)C(C)(C)C)cc1] |
| I-187 | 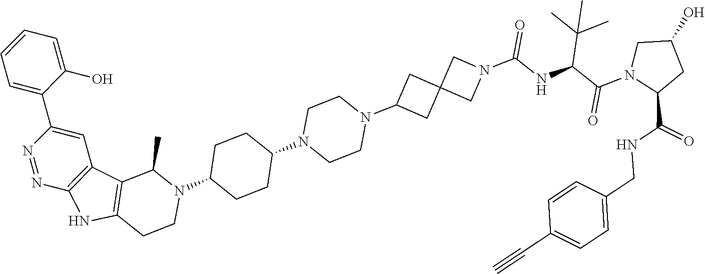 [SMILES: C#Cc1ccc(CNC(=O)[C@@H]2C[C@@H](O)CN2C(=O)[C@@H](NC(=O)N2CC3(CC(N4CCN([C@H]5CC[C@@H](N6CCc7[nH]c8nnc(-c9ccccc9O)cc8c7[C@H]6C)CC5)CC4)C3)C2)C(C)(C)C)cc1] |
| I-188 | 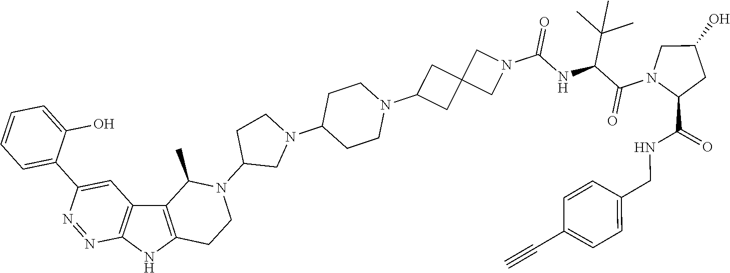 [SMILES: C#Cc1ccc(CNC(=O)[C@@H]2C[C@@H](O)CN2C(=O)[C@@H](NC(=O)N2CC3(CC(N4CCC(N5CCC(N6CCc7[nH]c8nnc(-c9ccccc9O)cc8c7[C@H]6C)C5)CC4)C3)C2)C(C)(C)C)cc1] |
| I-189 | 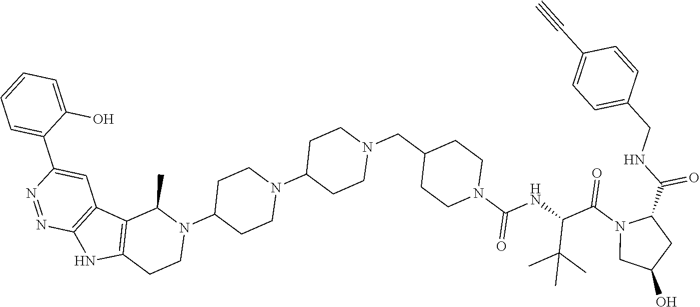 [SMILES: C#Cc1ccc(CNC(=O)[C@@H]2C[C@@H](O)CN2C(=O)[C@@H](NC(=O)N2CCC(CN3CCC(N4CCC(N5CCc6[nH]c7nnc(-c8ccccc8O)cc7c6[C@H]5C)CC4)CC3)CC2)C(C)(C)C)cc1] |
| I-190 | 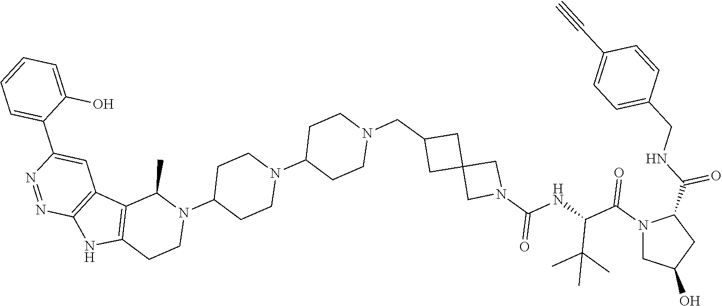 [SMILES: C#Cc1ccc(CNC(=O)[C@@H]2C[C@@H](O)CN2C(=O)[C@@H](NC(=O)N2CC3(CC(CN4CCC(N5CCC(N6CCc7[nH]c8nnc(-c9ccccc9O)cc8c7[C@H]6C)CC5)CC4)C3)C2)C(C)(C)C)cc1] |
| I-191 | 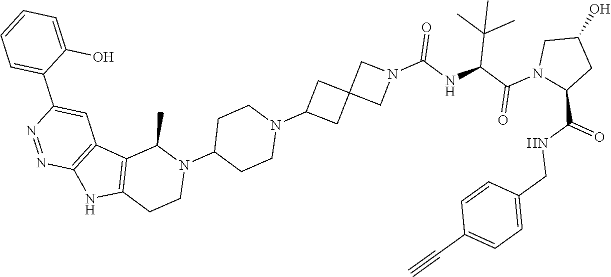 [SMILES: C#Cc1ccc(CNC(=O)[C@@H]2C[C@@H](O)CN2C(=O)[C@@H](NC(=O)N2CC3(CC(N4CCC(N5CCc6[nH]c7nnc(-c8ccccc8O)cc7c6[C@H]5C)CC4)C3)C2)C(C)(C)C)cc1] |
| I-192 | 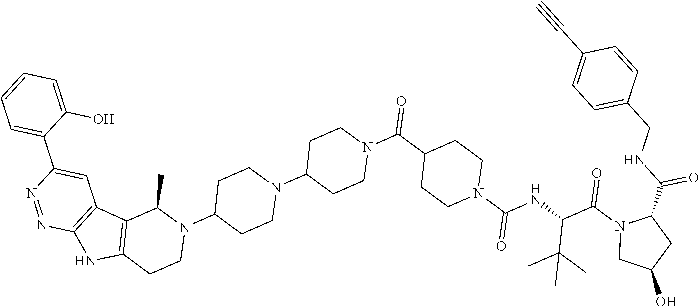 [SMILES: C#Cc1ccc(CNC(=O)[C@@H]2C[C@@H](O)CN2C(=O)[C@@H](NC(=O)N2CCC(C(=O)N3CCC(N4CCC(N5CCc6[nH]c7nnc(-c8ccccc8O)cc7c6[C@H]5C)CC4)CC3)CC2)C(C)(C)C)cc1] |
| I-193 | 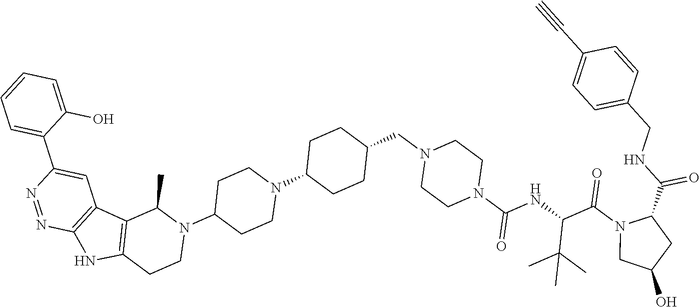 [SMILES: C#Cc1ccc(CNC(=O)[C@@H]2C[C@@H](O)CN2C(=O)[C@@H](NC(=O)N2CCN(C[C@H]3CC[C@@H](N4CCC(N5CCc6[nH]c7nnc(-c8ccccc8O)cc7c6[C@H]5C)CC4)CC3)CC2)C(C)(C)C)cc1] |
| I-194 | 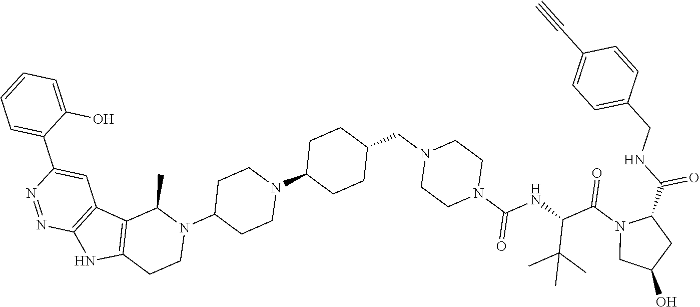 [SMILES: C#Cc1ccc(CNC(=O)[C@@H]2C[C@@H](O)CN2C(=O)[C@@H](NC(=O)N2CCN(C[C@H]3CC[C@H](N4CCC(N5CCc6[nH]c7nnc(-c8ccccc8O)cc7c6[C@H]5C)CC4)CC3)CC2)C(C)(C)C)cc1] |
| I-195 | 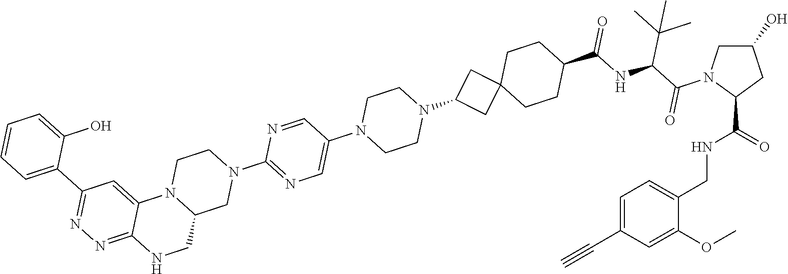 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)C)c(OC)c1] |
| I-196 | 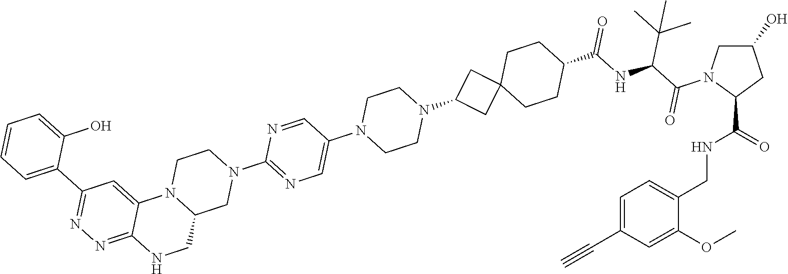 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)C)c(OC)c1] |
| I-197 | 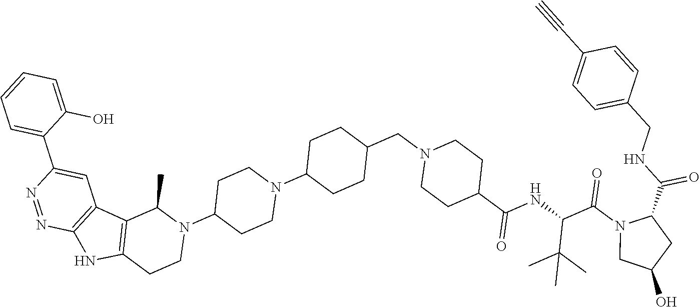 [SMILES: C#Cc1ccc(CNC(=O)[C@@H]2C[C@@H](O)CN2C(=O)[C@@H](NC(=O)C2CCN(CC3CCC(N4CCC(N5CCc6[nH]c7nnc(-c8ccccc8O)cc7c6[C@H]5C)CC4)CC3)CC2)C(C)(C)C)cc1] |
| I-198 | 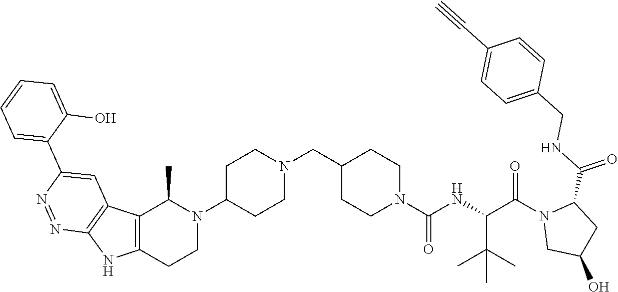 [SMILES: C#Cc1ccc(CNC(=O)[C@@H]2C[C@@H](O)CN2C(=O)[C@@H](NC(=O)N2CCC(CN3CCC(N4CCc5[nH]c6nnc(-c7ccccc7O)cc6c5[C@H]4C)CC3)CC2)C(C)(C)C)cc1] |
| I-199 | 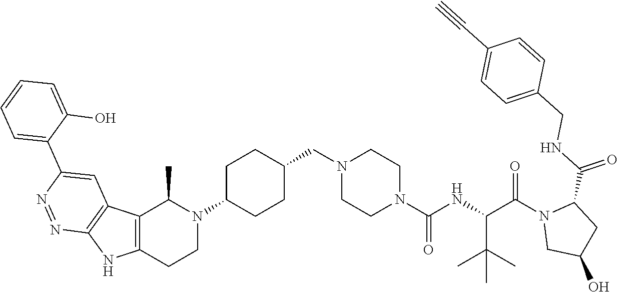 [SMILES: C#Cc1ccc(CNC(=O)[C@@H]2C[C@@H](O)CN2C(=O)[C@@H](NC(=O)N2CCN(C[C@H]3CC[C@@H](N4CCc5[nH]c6nnc(-c7ccccc7O)cc6c5[C@H]4C)CC3)CC2)C(C)(C)C)cc1] |
| I-200 | 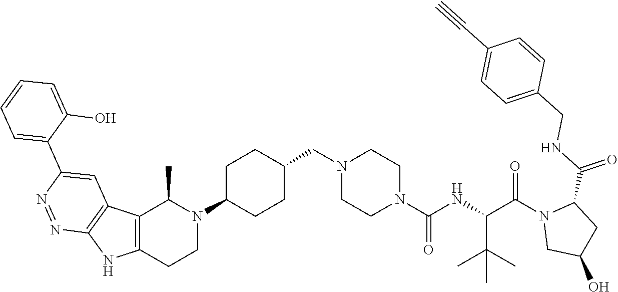 [SMILES: C#Cc1ccc(CNC(=O)[C@@H]2C[C@@H](O)CN2C(=O)[C@@H](NC(=O)N2CCN(C[C@H]3CC[C@H](N4CCc5[nH]c6nnc(-c7ccccc7O)cc6c5[C@H]4C)CC3)CC2)C(C)(C)C)cc1] |
| I-201 | 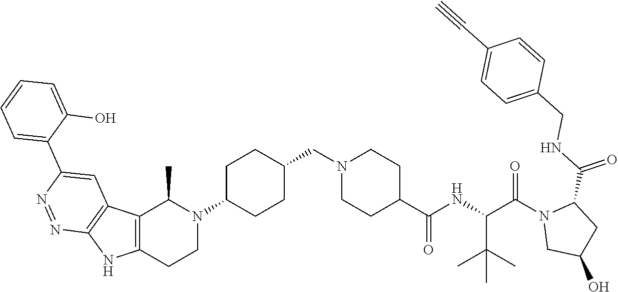 [SMILES: C#Cc1ccc(CNC(=O)[C@@H]2C[C@@H](O)CN2C(=O)[C@@H](NC(=O)C2CCN(C[C@H]3CC[C@@H](N4CCc5[nH]c6nnc(-c7ccccc7O)cc6c5[C@H]4C)CC3)CC2)C(C)(C)C)cc1] |
| I-202 | 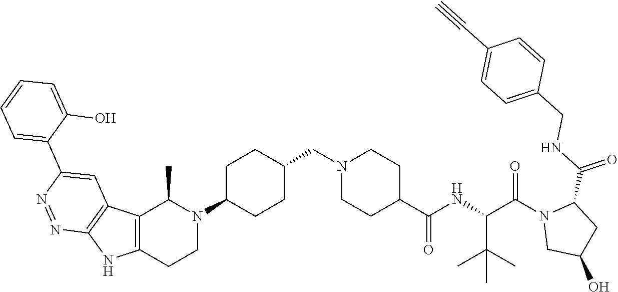 [SMILES: C#Cc1ccc(CNC(=O)[C@@H]2C[C@@H](O)CN2C(=O)[C@@H](NC(=O)C2CCN(C[C@H]3CC[C@H](N4CCc5[nH]c6nnc(-c7ccccc7O)cc6c5[C@H]4C)CC3)CC2)C(C)(C)C)cc1] |
| I-203 | 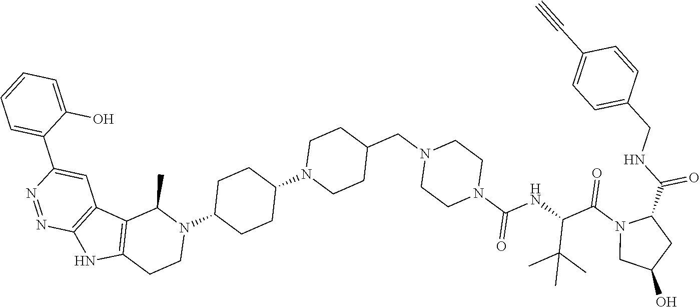 [SMILES: C#Cc1ccc(CNC(=O)[C@@H]2C[C@@H](O)CN2C(=O)[C@@H](NC(=O)N2CCN(CC3CCN([C@H]4CC[C@@H](N5CCc6[nH]c7nnc(-c8ccccc8O)cc7c6[C@H]5C)CC4)CC3)CC2)C(C)(C)C)cc1] |
| I-204 | 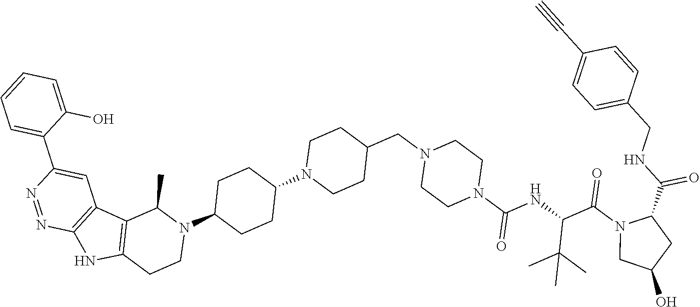 [SMILES: C#Cc1ccc(CNC(=O)[C@@H]2C[C@@H](O)CN2C(=O)[C@@H](NC(=O)N2CCN(CC3CCN([C@H]4CC[C@H](N5CCc6[nH]c7nnc(-c8ccccc8O)cc7c6[C@H]5C)CC4)CC3)CC2)C(C)(C)C)cc1] |
| I-205 | 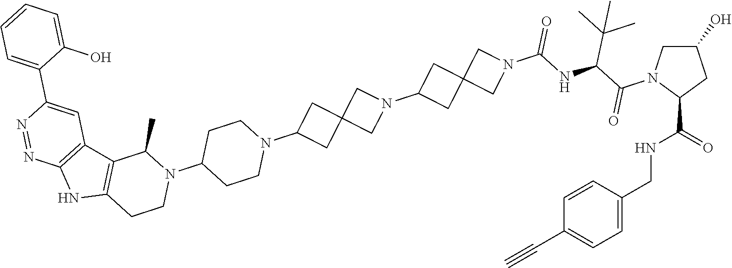 [SMILES: C#Cc1ccc(CNC(=O)[C@@H]2C[C@@H](O)CN2C(=O)[C@@H](NC(=O)N2CC3(CC(N4CC5(CC(N6CCC(N7CCc8[nH]c9nnc(-c%10ccccc%10O)cc9c8[C@H]7C)CC6)C5)C4)C3)C2)C(C)(C)C)cc1] |
| I-206 | 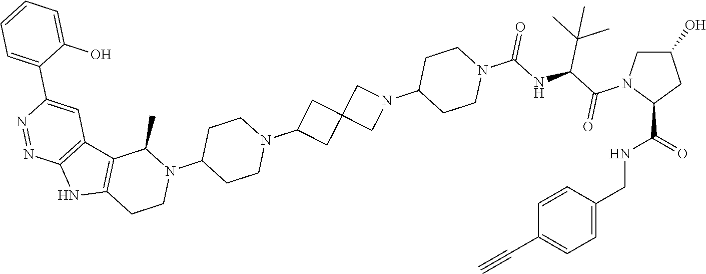 [SMILES: C#Cc1ccc(CNC(=O)[C@@H]2C[C@@H](O)CN2C(=O)[C@@H](NC(=O)N2CCC(N3CC4(CC(N5CCC(N6CCc7[nH]c8nnc(-c9ccccc9O)cc8c7[C@H]6C)CC5)C4)C3)CC2)C(C)(C)C)cc1] |
| I-207 | 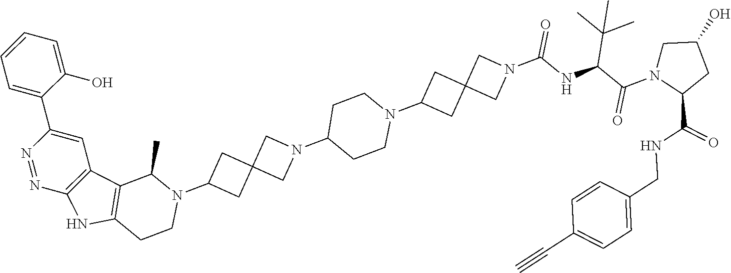 [SMILES: C#Cc1ccc(CNC(=O)[C@@H]2C[C@@H](O)CN2C(=O)[C@@H](NC(=O)N2CC3(CC(N4CCC(N5CC6(CC(N7CCc8[nH]c9nnc(-c%10ccccc%10O)cc9c8[C@H]7C)C6)C5)CC4)C3)C2)C(C)(C)C)cc1] |
| I-208 | 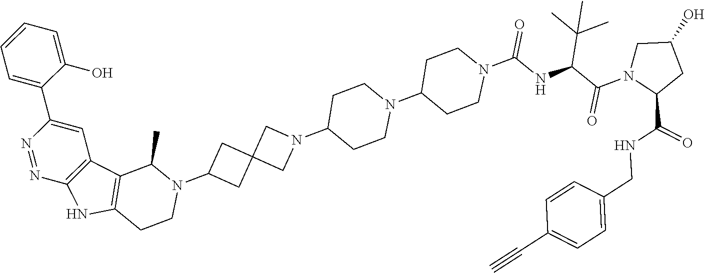 [SMILES: C#Cc1ccc(CNC(=O)[C@@H]2C[C@@H](O)CN2C(=O)[C@@H](NC(=O)N2CCC(N3CCC(N4CC5(CC(N6CCc7[nH]c8nnc(-c9ccccc9O)cc8c7[C@H]6C)C5)C4)CC3)CC2)C(C)(C)C)cc1] |
| I-209 | 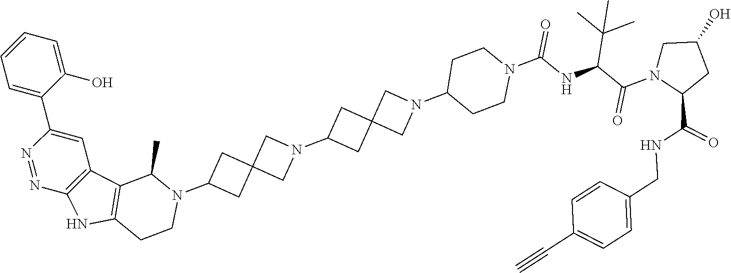 [SMILES: C#Cc1ccc(CNC(=O)[C@@H]2C[C@@H](O)CN2C(=O)[C@@H](NC(=O)N2CCC(N3CC4(CC(N5CC6(CC(N7CCc8[nH]c9nnc(-c%10ccccc%10O)cc9c8[C@H]7C)C6)C5)C4)C3)CC2)C(C)(C)C)cc1] |
| I-210 | 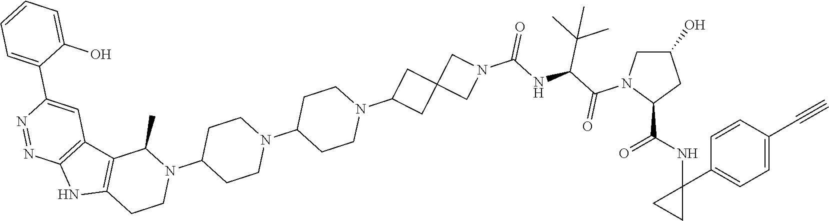 [SMILES: C#Cc1ccc(C2(NC(=O)[C@@H]3C[C@@H](O)CN3C(=O)[C@@H](NC(=O)N3CC4(CC(N5CCC(N6CCC(N7CCc8[nH]c9nnc(-c%10ccccc%10O)cc9c8[C@H]7C)CC6)CC5)C4)C3)C(C)(C)C)CC2)cc1] |
| I-211 | 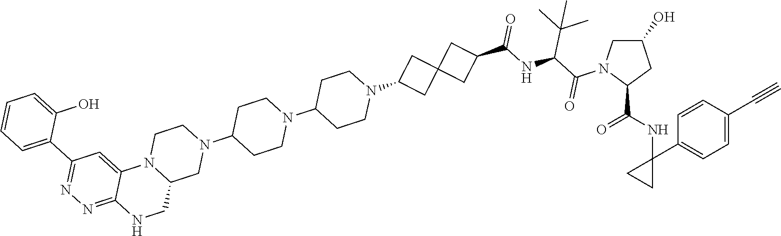 [SMILES: C#Cc1ccc(C2(NC(=O)[C@@H]3C[C@@H](O)CN3C(=O)[C@@H](NC(=O)[C@H]3CC4(C3)C[C@H](N3CCC(N5CCC(N6CCN7c8cc(-c9ccccc9O)nnc8NC[C@H]7C6)CC5)CC3)C4)C(C)(C)C)CC2)cc1] |
| I-212 | 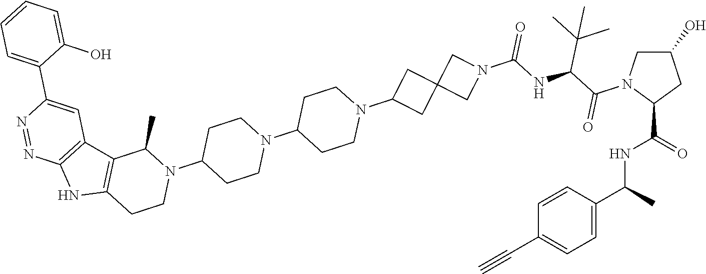 [SMILES: C#Cc1ccc([C@H](C)NC(=O)[C@@H]2C[C@@H](O)CN2C(=O)[C@@H](NC(=O)N2CC3(CC(N4CCC(N5CCC(N6CCc7[nH]c8nnc(-c9ccccc9O)cc8c7[C@H]6C)CC5)CC4)C3)C2)C(C)(C)C)cc1] |
| I-213 | 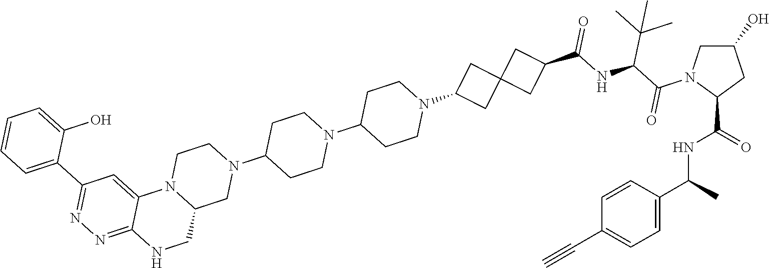 [SMILES: C#Cc1ccc([C@H](C)NC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)cc1] |
| I-214 | 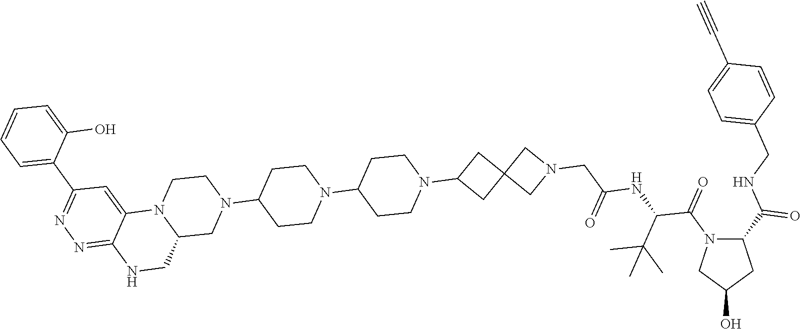 [SMILES: C#Cc1ccc(CNC(=O)[C@@H]2C[C@@H](O)CN2C(=O)[C@@H](NC(=O)CN2CC3(CC(N4CCC(N5CCC(N6CCN7c8cc(-c9ccccc9O)nnc8NC[C@H]7C6)CC5)CC4)C3)C2)C(C)(C)C)cc1] |
| I-215 | 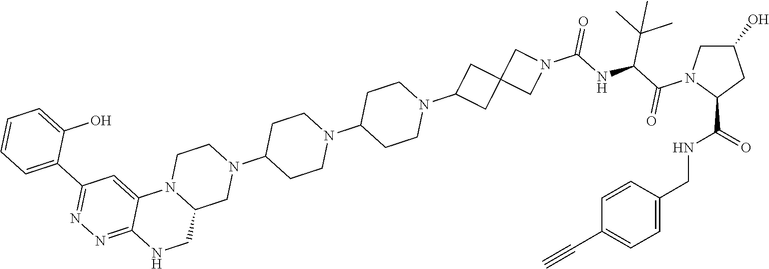 [SMILES: C#Cc1ccc(CNC(=O)[C@@H]2C[C@@H](O)CN2C(=O)[C@@H](NC(=O)N2CC3(CC(N4CCC(N5CCC(N6CCN7c8cc(-c9ccccc9O)nnc8NC[C@H]7C6)CC5)CC4)C3)C2)C(C)(C)C)cc1] |
| I-216 | 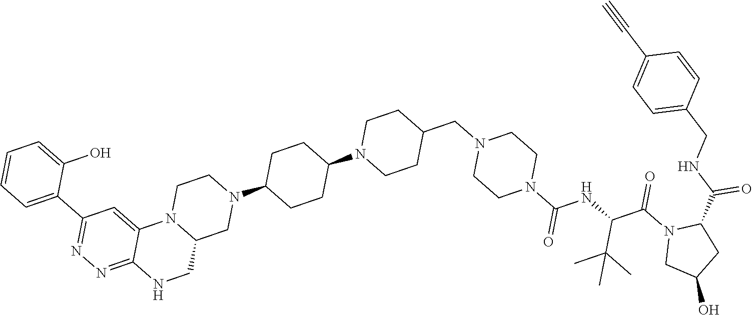 [SMILES: C#Cc1ccc(CNC(=O)[C@@H]2C[C@@H](O)CN2C(=O)[C@@H](NC(=O)N2CCN(CC3CCN([C@H]4CC[C@@H](N5CCN6c7cc(-c8ccccc8O)nnc7NC[C@H]6C5)CC4)CC3)CC2)C(C)(C)C)cc1] |
| I-217 | 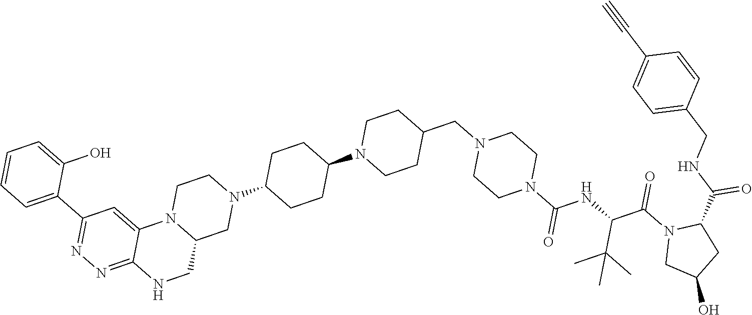 [SMILES: C#Cc1ccc(CNC(=O)[C@@H]2C[C@@H](O)CN2C(=O)[C@@H](NC(=O)N2CCN(CC3CCN([C@H]4CC[C@H](N5CCN6c7cc(-c8ccccc8O)nnc7NC[C@H]6C5)CC4)CC3)CC2)C(C)(C)C)cc1] |
| I-218 | 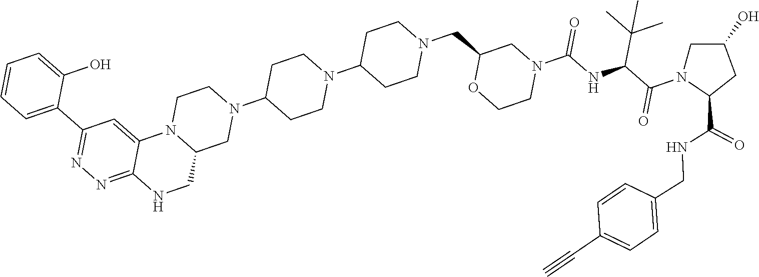 [SMILES: C#Cc1ccc(CNC(=O)[C@@H]2C[C@@H](O)CN2C(=O)[C@@H](NC(=O)N2CCO[C@@H](CN3CCC(N4CCC(N5CCN6c7cc(-c8ccccc8O)nnc7NC[C@H]6C5)CC4)CC3)C2)C(C)(C)C)cc1] |
| I-219 | 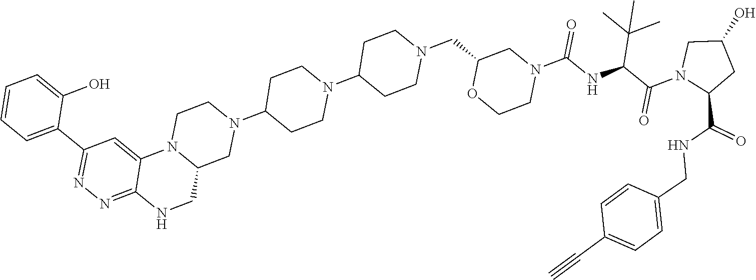 [SMILES: C#Cc1ccc(CNC(=O)[C@@H]2C[C@@H](O)CN2C(=O)[C@@H](NC(=O)N2CCO[C@H](CN3CCC(N4CCC(N5CCN6c7cc(-c8ccccc8O)nnc7NC[C@H]6C5)CC4)CC3)C2)C(C)(C)C)cc1] |
| I-220 | 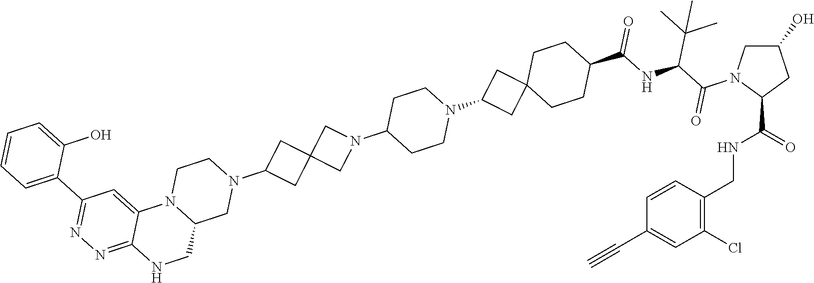 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CC5(CC(N6CCN7c8cc(-c9ccccc9O)nnc8NC[C@H]7C6)C5)C4)CC2)C3)C(C)(C)C)c(Cl)c1] |
| I-221 | 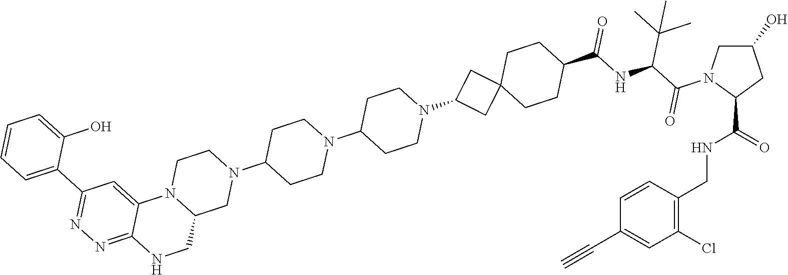 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CCC(N5CCN6c7cc(-c8ccccc8O)nnc7NC[C@H]6C5)CC4)CC2)C3)C(C)(C)C)c(Cl)c1] |
| I-222 | 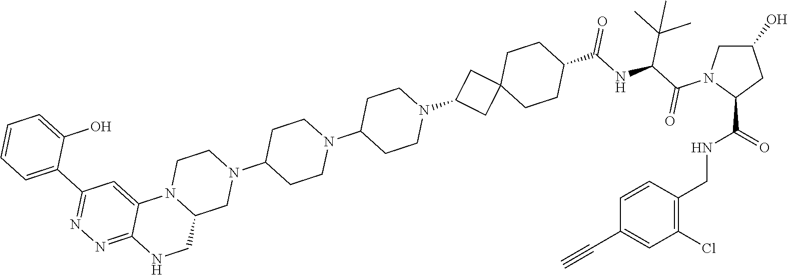 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CCC(N5CCN6c7cc(-c8ccccc8O)nnc7NC[C@H]6C5)CC4)CC2)C3)C(C)(C)C)c(Cl)c1] |
| I-223 | 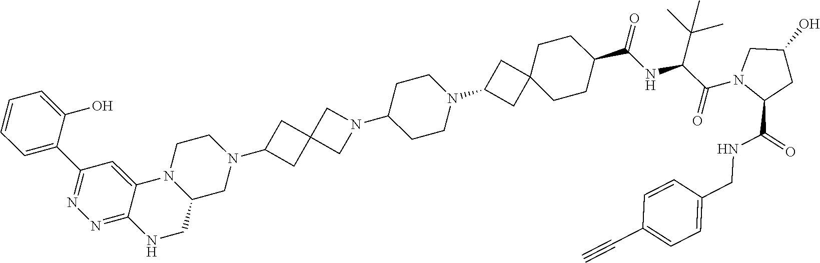 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CC5(CC(N6CCN7c8cc(-c9ccccc9O)nnc8NC[C@H]7C6)C5)C4)CC2)C3)C(C)(C)C)cc1] |
| I-224 | 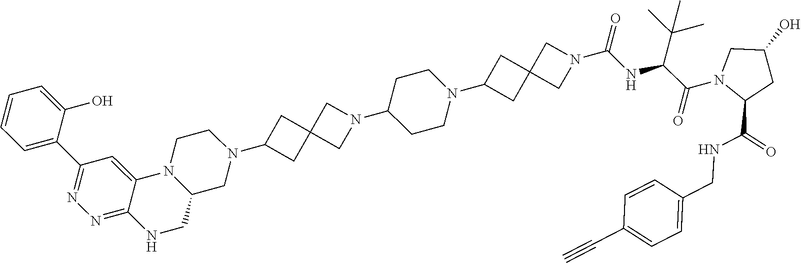 [SMILES: C#Cc1ccc(CNC(=O)[C@@H]2C[C@@H](O)CN2C(=O)[C@@H](NC(=O)N2CC3(CC(N4CCC(N5CC6(CC(N7CCN8c9cc(-c%10ccccc%10O)nnc9NC[C@H]8C7)C6)C5)CC4)C3)C2)C(C)(C)C)cc1] |
| I-225 | 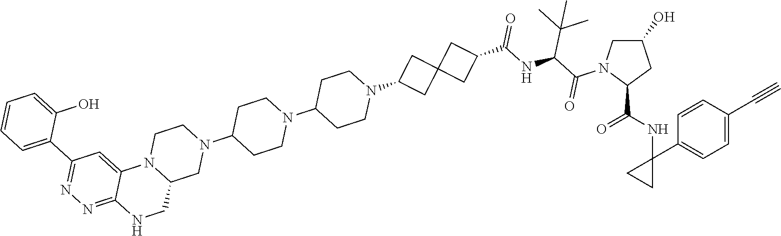 [SMILES: C#Cc1ccc(C2(NC(=O)[C@@H]3C[C@@H](O)CN3C(=O)[C@@H](NC(=O)[C@H]3CC4(C3)C[C@H](N3CCC(N5CCC(N6CCN7c8cc(-c9ccccc9O)nnc8NC[C@H]7C6)CC5)CC3)C4)C(C)(C)C)CC2)cc1] |
| I-226 | 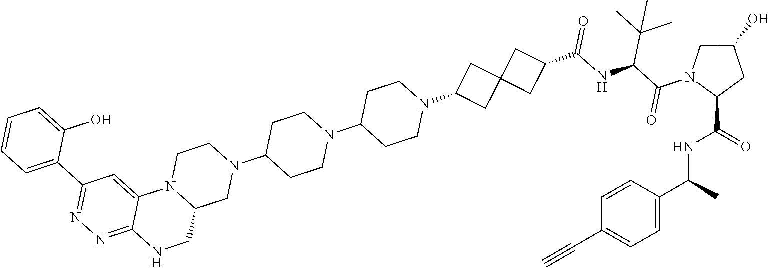 [SMILES: C#Cc1ccc([C@H](C)NC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)cc1] |
| I-227 | 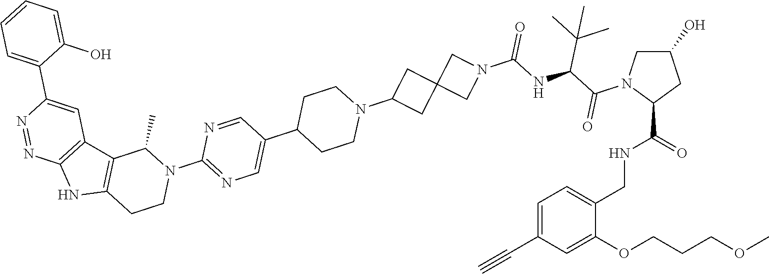 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@@H]6C)nc5)CC4)C3)C2)C(C)(C)C)c(OCCCOC)c1] |
| I-228 | 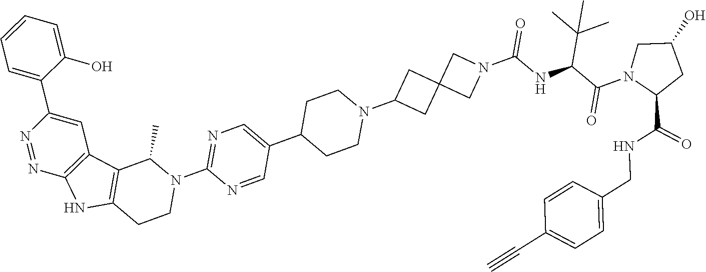 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@@H]6C)nc5)CC4)C3)C2)C(C)(C)C)cc1] |
| I-229 | 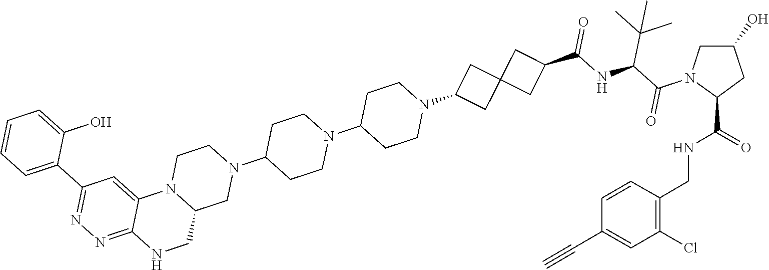 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(Cl)c1] |
| I-230 | 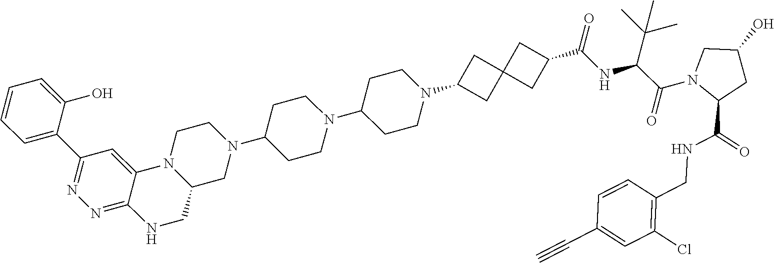 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(Cl)c1] |
| I-231 | 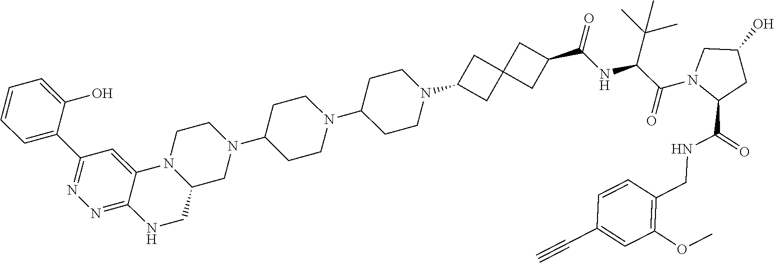 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(OC)c1] |
| I-232 | 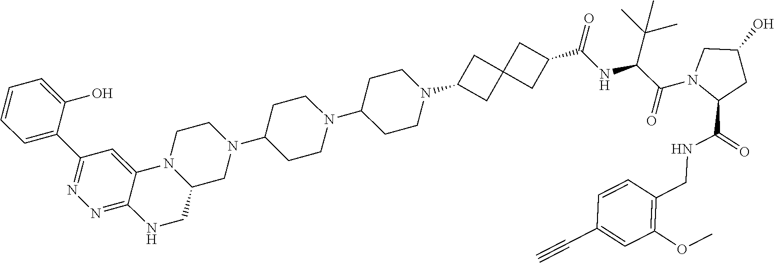 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(OC)c1] |
| I-233 | 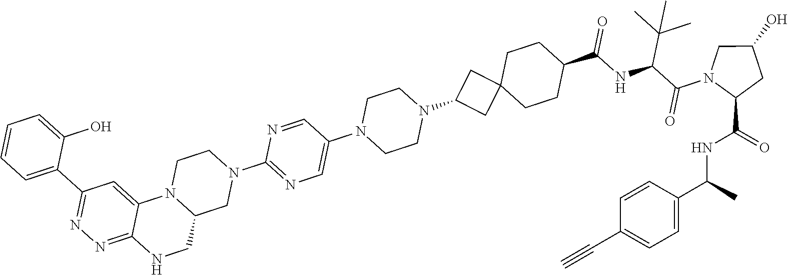 [SMILES: C#Cc1ccc([C@H](C)NC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)C)cc1] |
| I-234 | 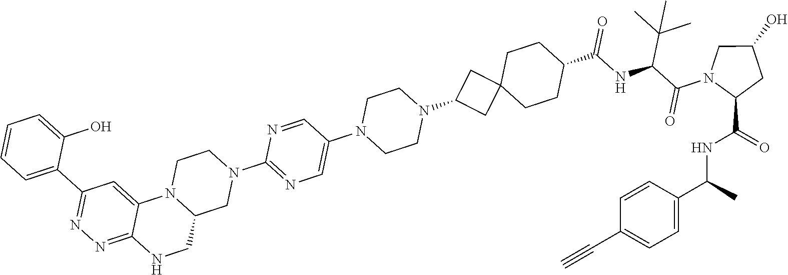 [SMILES: C#Cc1ccc([C@H](C)NC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)C)cc1] |
| I-235 | 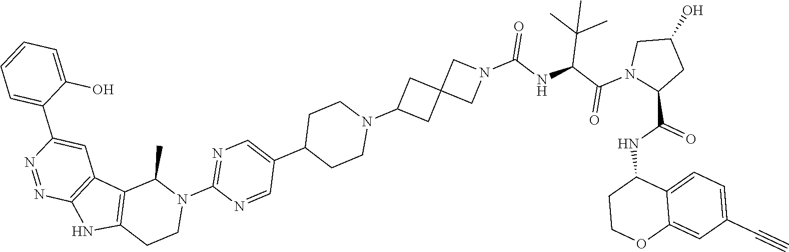 [SMILES: C#Cc1ccc2c(c1)OCC[C@@H]2NC(=O)[C@@H]1C[C@@H](O)CN1C(=O)[C@@H](NC(=O)N1CC2(CC(N3CCC(c4cnc(N5CCc6[nH]c7nnc(-c8ccccc8O)cc7c6[C@H]5C)nc4)CC3)C2)C1)C(C)(C)C] |
| I-236 | 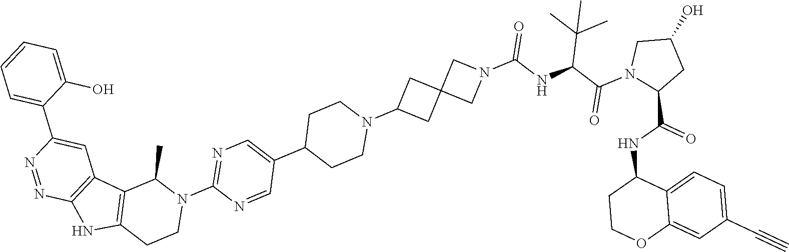 [SMILES: C#Cc1ccc2c(c1)OCC[C@H]2NC(=O)[C@@H]1C[C@@H](O)CN1C(=O)[C@@H](NC(=O)N1CC2(CC(N3CCC(c4cnc(N5CCc6[nH]c7nnc(-c8ccccc8O)cc7c6[C@H]5C)nc4)CC3)C2)C1)C(C)(C)C] |
| I-237 | 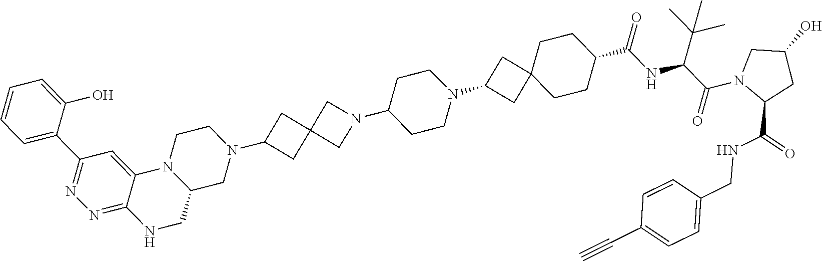 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CC5(CC(N6CCN7c8cc(-c9ccccc9O)nnc8NC[C@H]7C6)C5)C4)CC2)C3)C(C)(C)C)cc1] |
| I-238 | 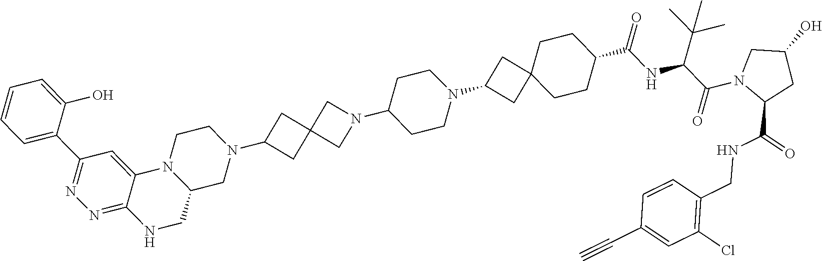 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CC5(CC(N6CCN7c8cc(-c9ccccc9O)nnc8NC[C@H]7C6)C5)C4)CC2)C3)C(C)(C)C)c(Cl)c1] |
| I-239 | 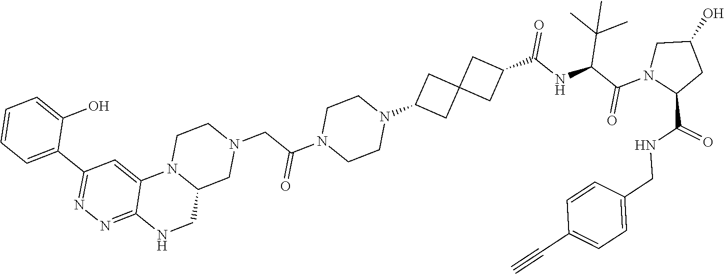 [SMILES: C#Cc1ccc(CNC(=O)[C@@H]2C[C@@H](O)CN2C(=O)[C@@H](NC(=O)[C@H]2CC3(C2)C[C@H](N2CCN(C(=O)CN4CCN5c6cc(-c7ccccc7O)nnc6NC[C@H]5C4)CC2)C3)C(C)(C)C)cc1] |
| I-240 | 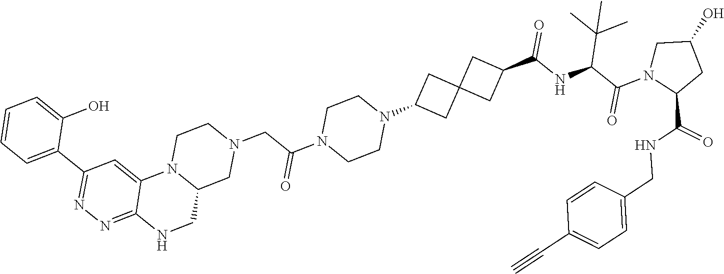 [SMILES: C#Cc1ccc(CNC(=O)[C@@H]2C[C@@H](O)CN2C(=O)[C@@H](NC(=O)[C@H]2CC3(C2)C[C@H](N2CCN(C(=O)CN4CCN5c6cc(-c7ccccc7O)nnc6NC[C@H]5C4)CC2)C3)C(C)(C)C)cc1] |
| I-241 | 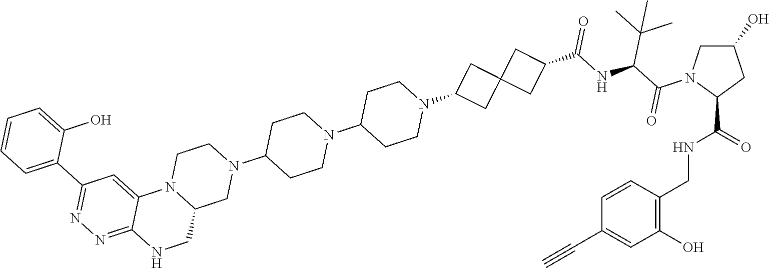 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(O)c1] |
| I-242 | 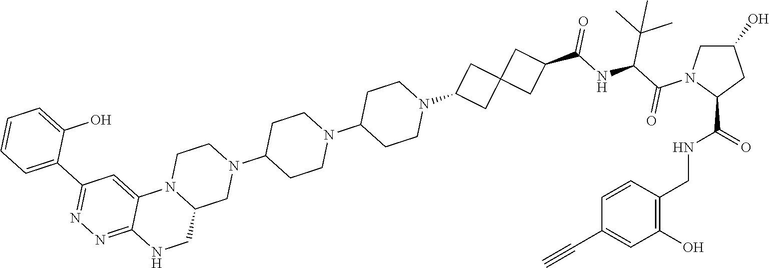 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(O)c1] |
| I-243 | 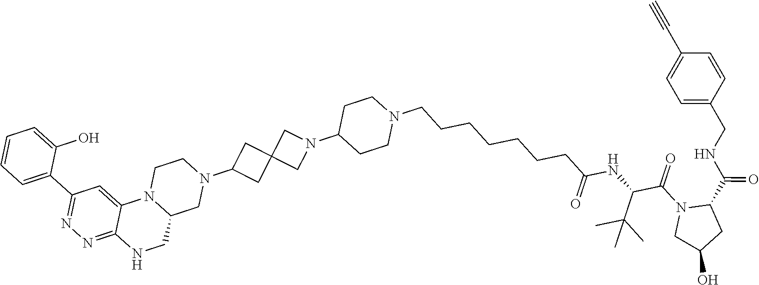 [SMILES: C#Cc1ccc(CNC(=O)[C@@H]2C[C@@H](O)CN2C(=O)[C@@H](NC(=O)CCCCCCCN2CCC(N3CC4(CC(N5CCN6c7cc(-c8ccccc8O)nnc7NC[C@H]6C5)C4)C3)CC2)C(C)(C)C)cc1] |
| I-244 | 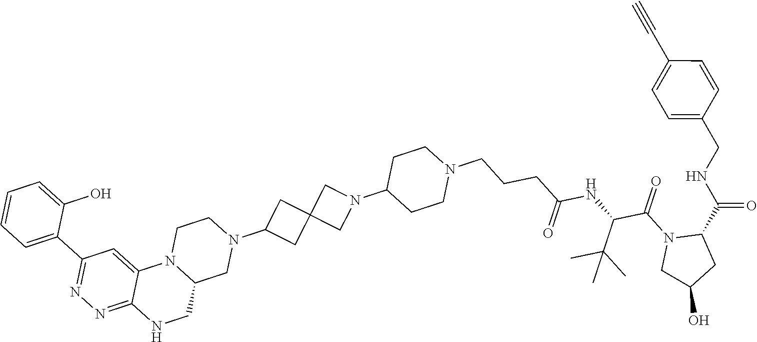 [SMILES: C#Cc1ccc(CNC(=O)[C@@H]2C[C@@H](O)CN2C(=O)[C@@H](NC(=O)CCCN2CCC(N3CC4(CC(N5CCN6c7cc(-c8ccccc8O)nnc7NC[C@H]6C5)C4)C3)CC2)C(C)(C)C)cc1] |
| I-245 | 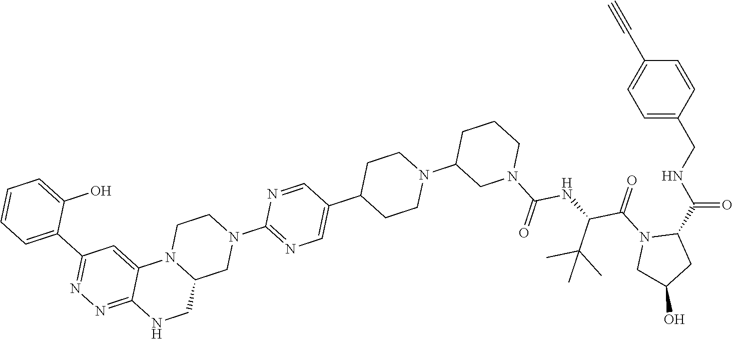 [SMILES: C#Cc1ccc(CNC(=O)[C@@H]2C[C@@H](O)CN2C(=O)[C@@H](NC(=O)N2CCCC(N3CCC(c4cnc(N5CCN6c7cc(-c8ccccc8O)nnc7NC[C@H]6C5)nc4)CC3)C2)C(C)(C)C)cc1] |
| I-246 | 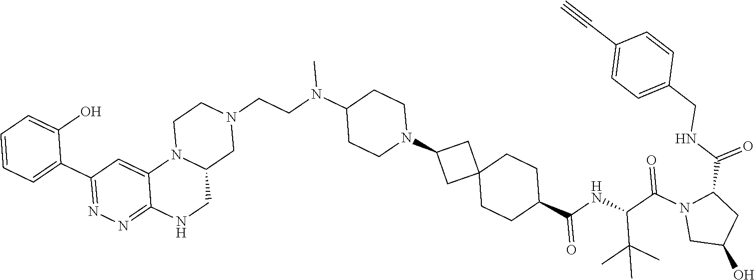 [SMILES: C#Cc1ccc(CNC(=O)[C@@H]2C[C@@H](O)CN2C(=O)[C@@H](NC(=O)[C@H]2CCC3(CC2)C[C@H](N2CCC(N(C)CCN4CCN5c6cc(-c7ccccc7O)nnc6NC[C@H]5C4)CC2)C3)C(C)(C)C)cc1] |
| I-247 | 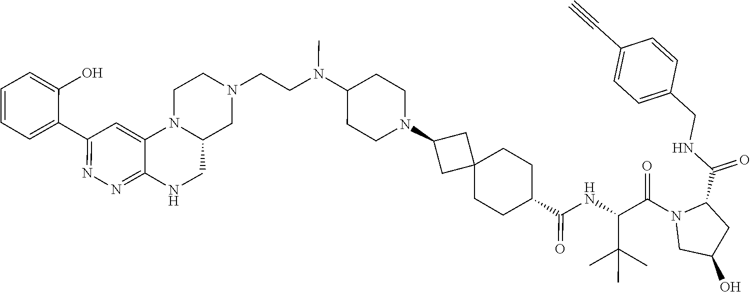 [SMILES: C#Cc1ccc(CNC(=O)[C@@H]2C[C@@H](O)CN2C(=O)[C@@H](NC(=O)[C@H]2CCC3(CC2)C[C@H](N2CCC(N(C)CCN4CCN5c6cc(-c7ccccc7O)nnc6NC[C@H]5C4)CC2)C3)C(C)(C)C)cc1] |
| I-248 | 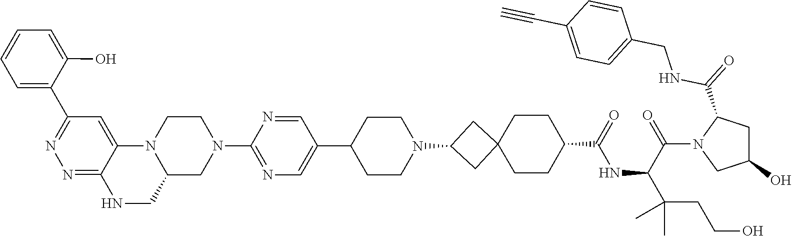 [SMILES: C#Cc1ccc(CNC(=O)[C@@H]2C[C@@H](O)CN2C(=O)[C@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)CCO)cc1] |
| I-249 | 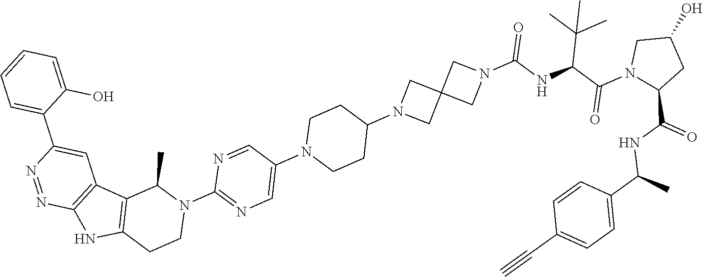 [SMILES: C#Cc1ccc([C@H](C)NC(=O)[C@@H]2C[C@@H](O)CN2C(=O)[C@@H](NC(=O)N2CC3(C2)CN(C2CCN(c4cnc(N5CCc6[nH]c7nnc(-c8ccccc8O)cc7c6[C@H]5C)nc4)CC2)C3)C(C)(C)C)cc1] |
| I-250 | 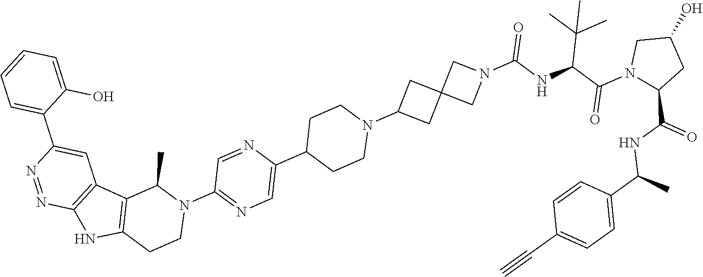 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)cn5)CC4)C3)C2)C(C)(C)C)cc1] |
| I-251 | 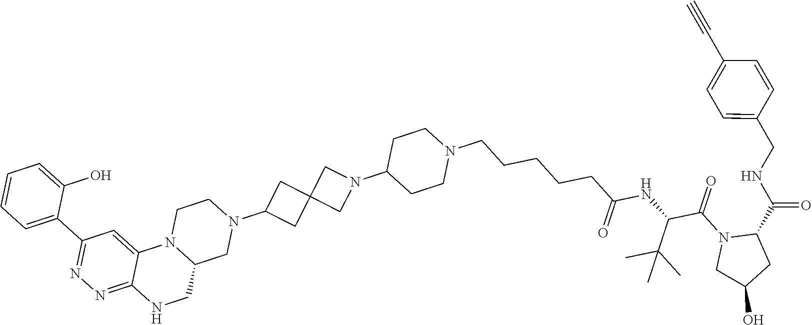 [SMILES: C#Cc1ccc(CNC(=O)[C@@H]2C[C@@H](O)CN2C(=O)[C@@H](NC(=O)CCCCCN2CCC(N3CC4(CC(N5CCN6c7cc(-c8ccccc8O)nnc7NC[C@H]6C5)C4)C3)CC2)C(C)(C)C)cc1] |
| I-252 | 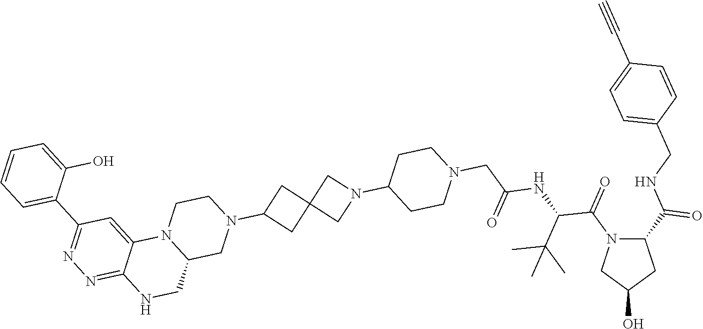 [SMILES: C#Cc1ccc(CNC(=O)[C@@H]2C[C@@H](O)CN2C(=O)[C@@H](NC(=O)CN2CCC(N3CC4(CC(N5CCN6c7cc(-c8ccccc8O)nnc7NC[C@H]6C5)C4)C3)CC2)C(C)(C)C)cc1] |
| I-253 | 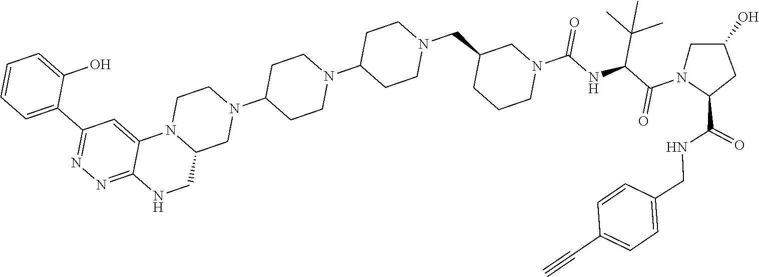 [SMILES: C#Cc1ccc(CNC(=O)[C@@H]2C[C@@H](O)CN2C(=O)[C@@H](NC(=O)N2CCC[C@@H](CN3CCC(N4CCC(N5CCN6c7cc(-c8ccccc8O)nnc7NC[C@H]6C5)CC4)CC3)C2)C(C)(C)C)cc1] |
| I-254 | 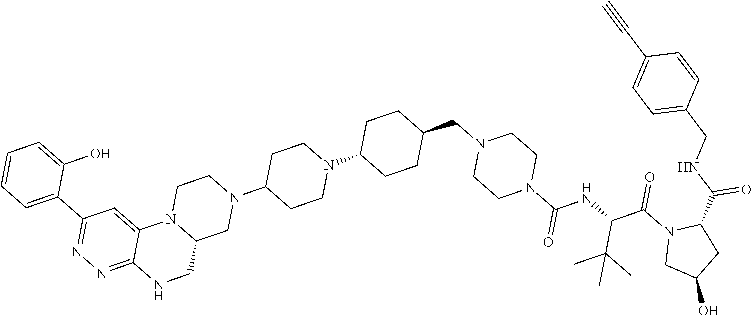 [SMILES: C#Cc1ccc(CNC(=O)[C@@H]2C[C@@H](O)CN2C(=O)[C@@H](NC(=O)N2CCN(C[C@H]3CC[C@H](N4CCC(N5CCN6c7cc(-c8ccccc8O)nnc7NC[C@H]6C5)CC4)CC3)CC2)C(C)(C)C)cc1] |
| I-255 | 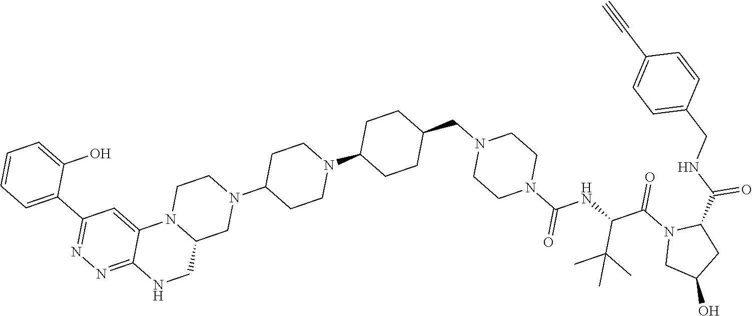 [SMILES: C#Cc1ccc(CNC(=O)[C@@H]2C[C@@H](O)CN2C(=O)[C@@H](NC(=O)N2CCN(C[C@H]3CC[C@@H](N4CCC(N5CCN6c7cc(-c8ccccc8O)nnc7NC[C@H]6C5)CC4)CC3)CC2)C(C)(C)C)cc1] |
| I-256 | 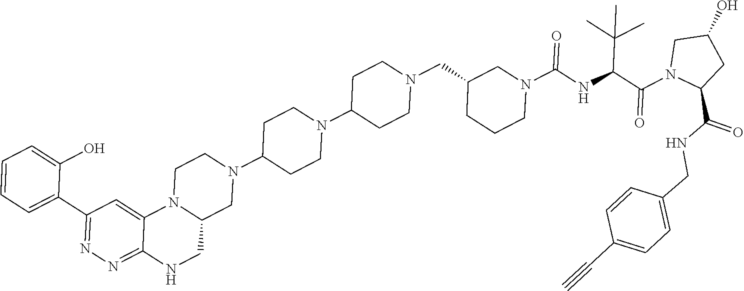 [SMILES: C#Cc1ccc(CNC(=O)[C@@H]2C[C@@H](O)CN2C(=O)[C@@H](NC(=O)N2CCC[C@H](CN3CCC(N4CCC(N5CCN6c7cc(-c8ccccc8O)nnc7NC[C@H]6C5)CC4)CC3)C2)C(C)(C)C)cc1] |
| I-257 | 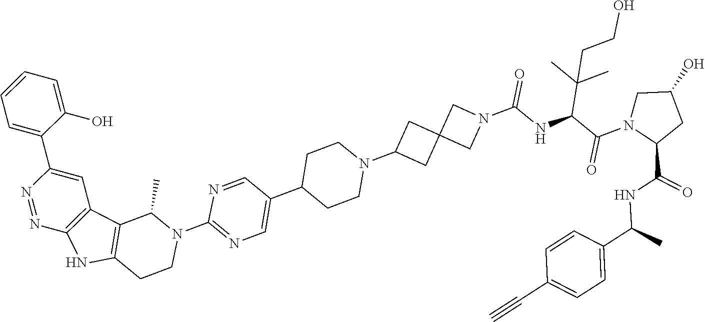 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@@H]6C)nc5)CC4)C3)C2)C(C)(C)CCO)cc1] |
| I-258 | 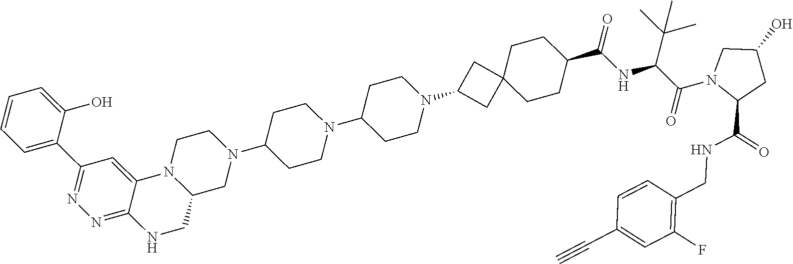 [SMILES: C#Cc1ccc(CNC(=O)[C@@H]2C[C@@H](O)CN2C(=O)[C@@H](NC(=O)[C@H]2CCC3(CC2)C[C@H](N2CCC(N4CCC(N5CCN6c7cc(-c8ccccc8O)nnc7NC[C@H]6C5)CC4)CC2)C3)C(C)(C)C)c(F)c1] |
| I-259 | 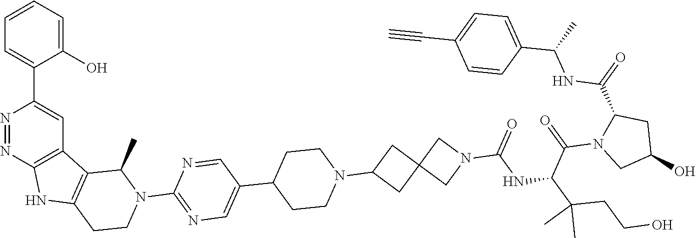 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCO)cc1] |
| I-262 | 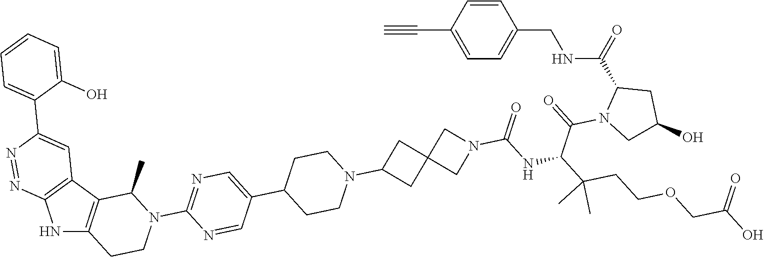 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCOCC(=O)O)cc1] |
| I-263 | 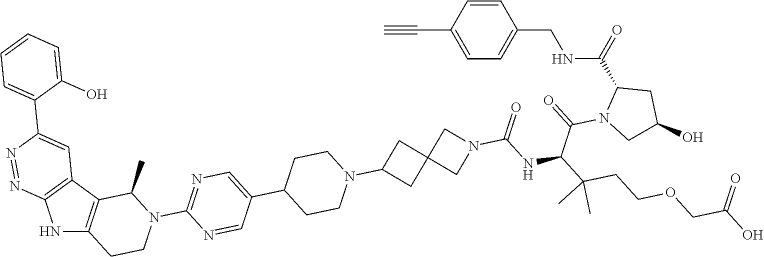 [SMILES: C#Cc1ccc(CNC(=O)[C@@H]2C[C@@H](O)CN2C(=O)[C@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCOCC(=O)O)cc1] |
| I-266 | 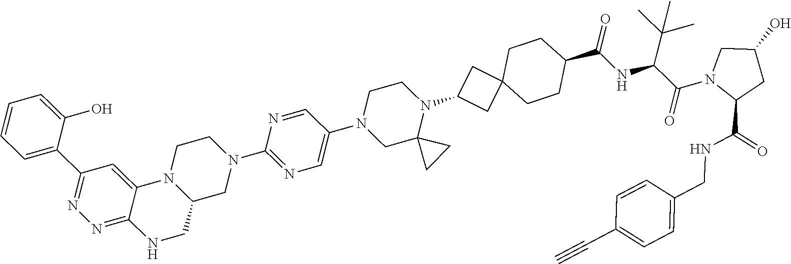 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC24CC4)C3)C(C)(C)C)cc1] |
| I-267 | 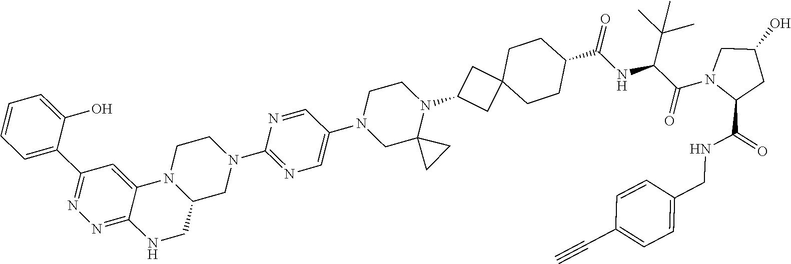 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC24CC4)C3)C(C)(C)C)cc1] |
| I-268 | 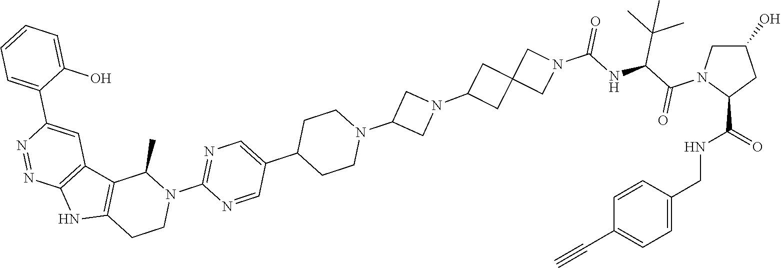 [SMILES: C#Cc1ccc(CNC(=O)[C@@H]2C[C@@H](O)CN2C(=O)[C@@H](NC(=O)N2CC3(CC(N4CC(N5CCC(c6cnc(N7CCc8[nH]c9nnc(-c%10ccccc%10O)cc9c8[C@H]7C)nc6)CC5)C4)C3)C2)C(C)(C)C)cc1] |
| I-269 | 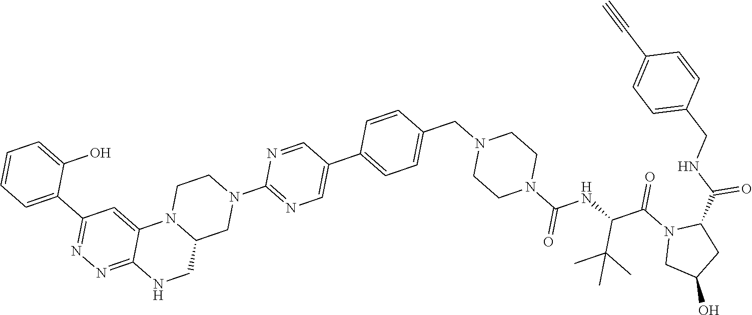 [SMILES: C#Cc1ccc(CNC(=O)[C@@H]2C[C@@H](O)CN2C(=O)[C@@H](NC(=O)N2CCN(Cc3ccc(-c4cnc(N5CCN6c7cc(-c8ccccc8O)nnc7NC[C@H]6C5)nc4)cc3)CC2)C(C)(C)C)cc1] |
| I-270 | 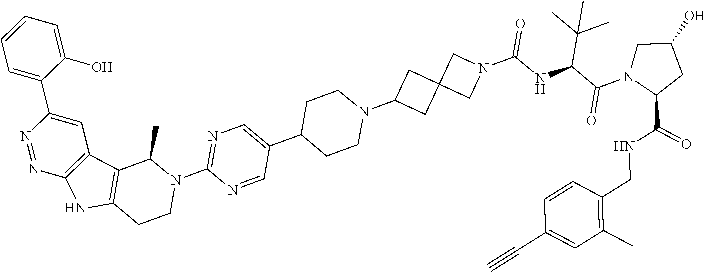 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(C)c1] |
| I-271 | 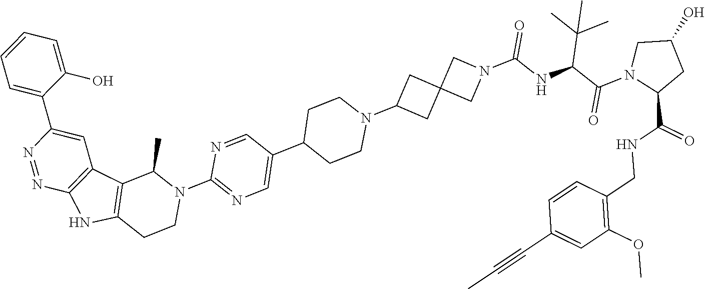 [SMILES: CC#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(OC)c1] |
| I-272 | 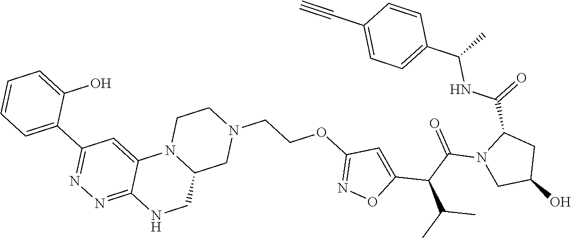 [SMILES: C#Cc1ccc([C@H](C)NC(=O)[C@@H]2C[C@@H](O)CN2C(=O)[C@H](c2cc(OCCN3CCN4c5cc(-c6ccccc6O)nnc5NC[C@H]4C3)no2)C(C)C)cc1] |
| I-273 | 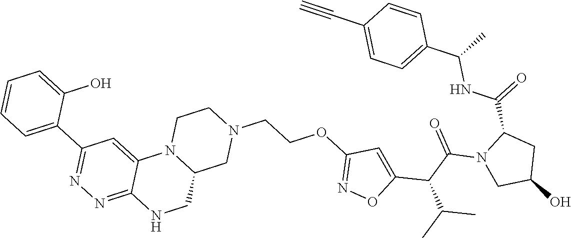 [SMILES: C#Cc1ccc([C@H](C)NC(=O)[C@@H]2C[C@@H](O)CN2C(=O)[C@@H](c2cc(OCCN3CCN4c5cc(-c6ccccc6O)nnc5NC[C@H]4C3)no2)C(C)C)cc1] |
| I-274 | 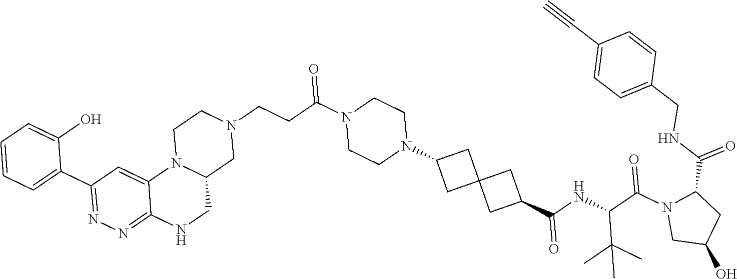 [SMILES: C#Cc1ccc(CNC(=O)[C@@H]2C[C@@H](O)CN2C(=O)[C@@H](NC(=O)[C@H]2CC3(C2)C[C@H](N2CCN(C(=O)CCN4CCN5c6cc(-c7ccccc7O)nnc6NC[C@H]5C4)CC2)C3)C(C)(C)C)cc1] |
| I-275 | 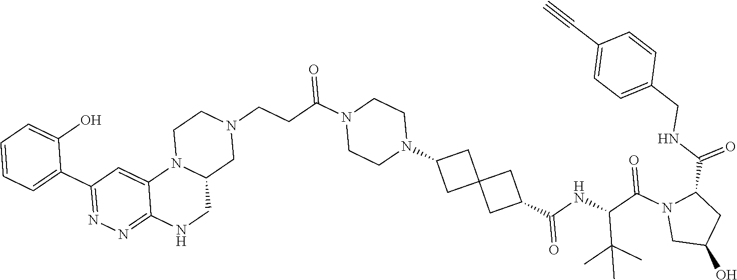 [SMILES: C#Cc1ccc(CNC(=O)[C@@H]2C[C@@H](O)CN2C(=O)[C@@H](NC(=O)[C@H]2CC3(C2)C[C@H](N2CCN(C(=O)CCN4CCN5c6cc(-c7ccccc7O)nnc6NC[C@H]5C4)CC2)C3)C(C)(C)C)cc1] |
| I-276 | 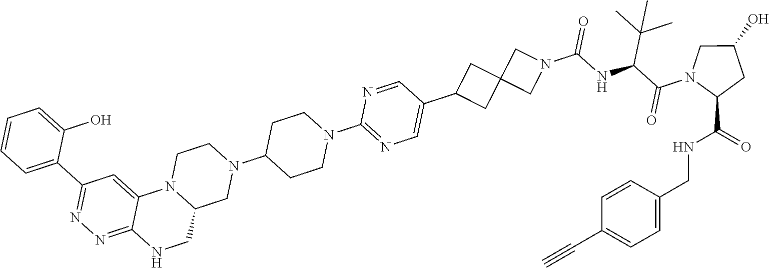 [SMILES: C#Cc1ccc(CNC(=O)[C@@H]2C[C@@H](O)CN2C(=O)[C@@H](NC(=O)N2CC3(CC(c4cnc(N5CCC(N6CCN7c8cc(-c9ccccc9O)nnc8NC[C@H]7C6)CC5)nc4)C3)C2)C(C)(C)C)cc1] |
| I-277 | 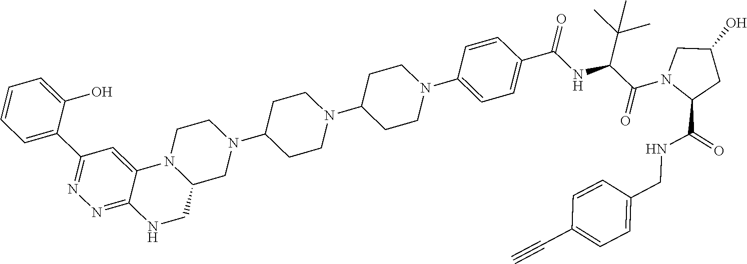 [SMILES: C#Cc1ccc(CNC(=O)[C@@H]2C[C@@H](O)CN2C(=O)[C@@H](NC(=O)c2ccc(N3CCC(N4CCC(N5CCN6c7cc(-c8ccccc8O)nnc7NC[C@H]6C5)CC4)CC3)cc2)C(C)(C)C)cc1] |
| I-281 | 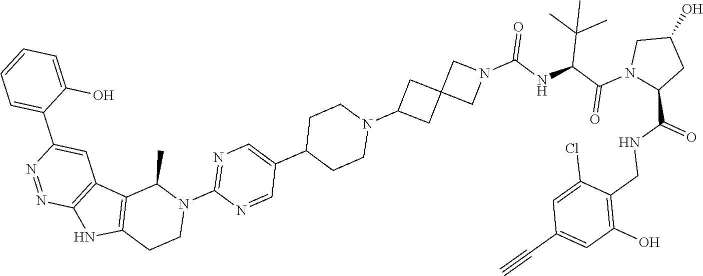 [SMILES: C#Cc1cc(O)c(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(Cl)c1] |
| I-282 | 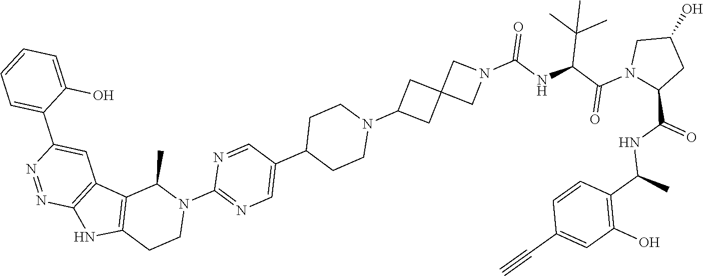 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(O)c1] |
| I-283 | 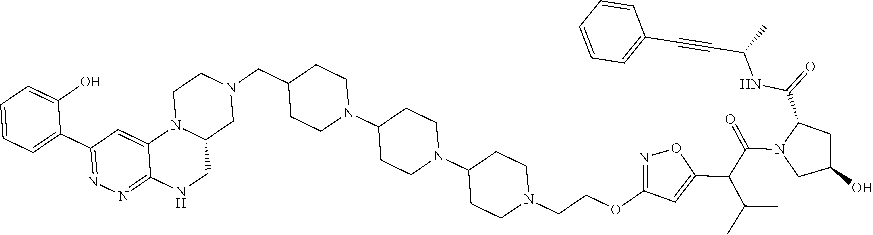 [SMILES: CC(C)C(C(=O)N1C[C@H](O)C[C@H]1C(=O)N[C@@H](C)C#Cc1ccccc1)c1cc(OCCN2CCC(N3CCC(N4CCC(CN5CCN6c7cc(-c8ccccc8O)nnc7NC[C@H]6C5)CC4)CC3)CC2)no1] |
| I-284 | 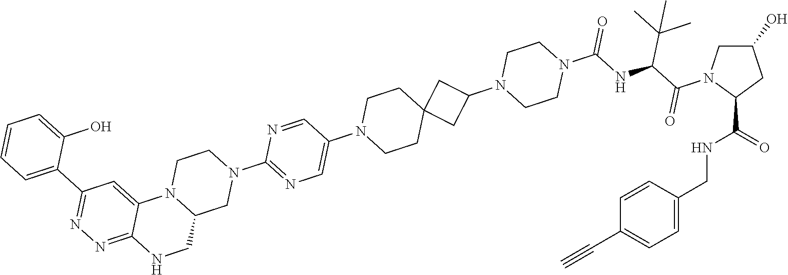 [SMILES: C#Cc1ccc(CNC(=O)[C@@H]2C[C@@H](O)CN2C(=O)[C@@H](NC(=O)N2CCN(C3CC4(CCN(c5cnc(N6CCN7c8cc(-c9ccccc9O)nnc8NC[C@H]7C6)nc5)CC4)C3)CC2)C(C)(C)C)cc1] |
| I-285 | 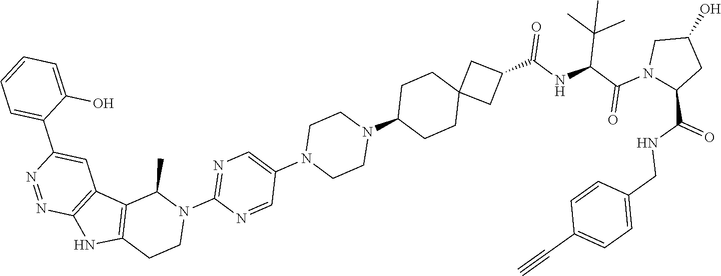 [SMILES: C#Cc1ccc(CNC(=O)[C@@H]2C[C@@H](O)CN2C(=O)[C@@H](NC(=O)[C@H]2CC3(CC[C@H](N4CCN(c5cnc(N6CCc7[nH]c8nnc(-c9ccccc9O)cc8c7[C@H]6C)nc5)CC4)CC3)C2)C(C)(C)C)cc1] |
| I-286 | 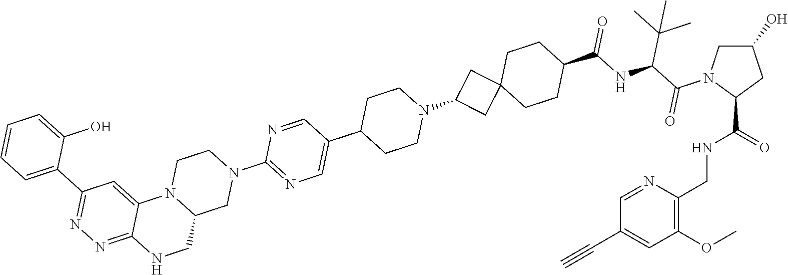 [SMILES: C#Cc1cnc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(OC)c1] |
| I-287 | 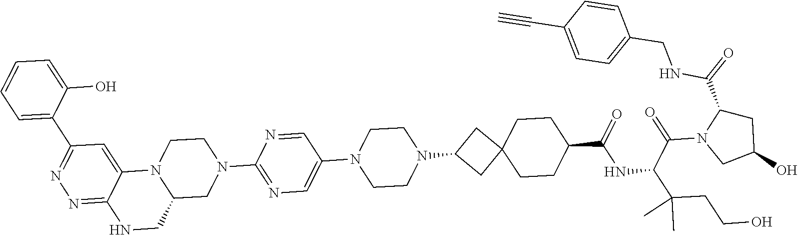 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)CCO)cc1] |
| I-288 | 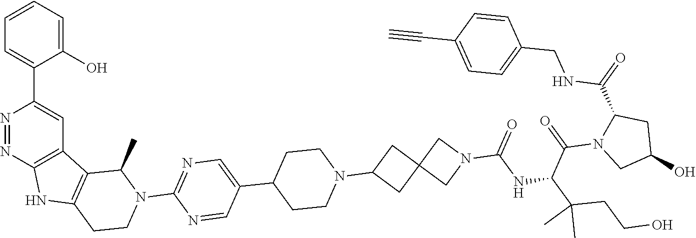 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCO)cc1] |
| I-291 | 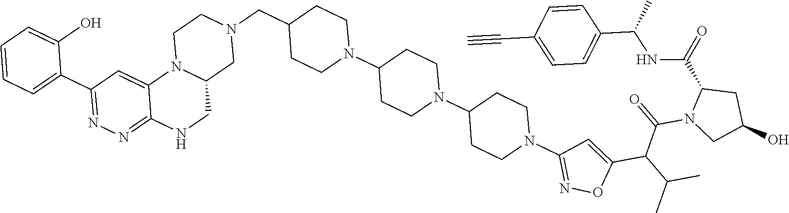 [SMILES: C#Cc1ccc([C@H](C)NC(=O)[C@@H]2C[C@@H](O)CN2C(=O)C(c2cc(N3CCC(N4CCC(N5CCC(CN6CCN7c8cc(-c9ccccc9O)nnc8NC[C@H]7C6)CC5)CC4)CC3)no2)C(C)C)cc1] |
| I-292 | 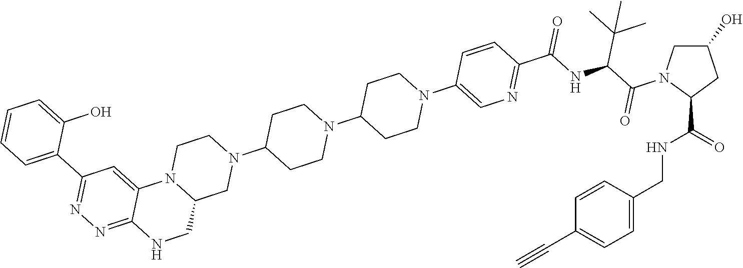 [SMILES: C#Cc1ccc(CNC(=O)[C@@H]2C[C@@H](O)CN2C(=O)[C@@H](NC(=O)c2ccc(N3CCC(N4CCC(N5CCN6c7cc(-c8ccccc8O)nnc7NC[C@H]6C5)CC4)CC3)cn2)C(C)(C)C)cc1] |
| I-293 | 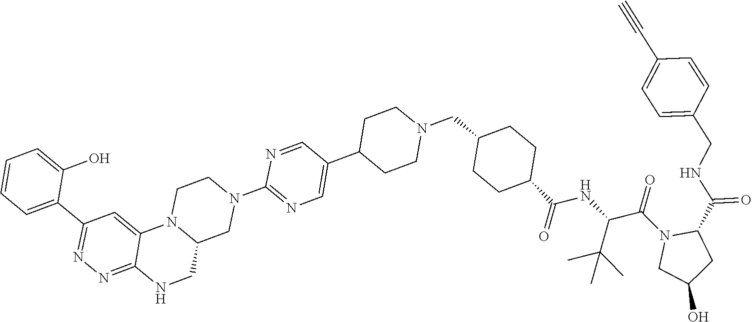 [SMILES: C#Cc1ccc(CNC(=O)[C@@H]2C[C@@H](O)CN2C(=O)[C@@H](NC(=O)[C@H]2CC[C@@H](CN3CCC(c4cnc(N5CCN6c7cc(-c8ccccc8O)nnc7NC[C@H]6C5)nc4)CC3)CC2)C(C)(C)C)cc1] |
| I-294 | 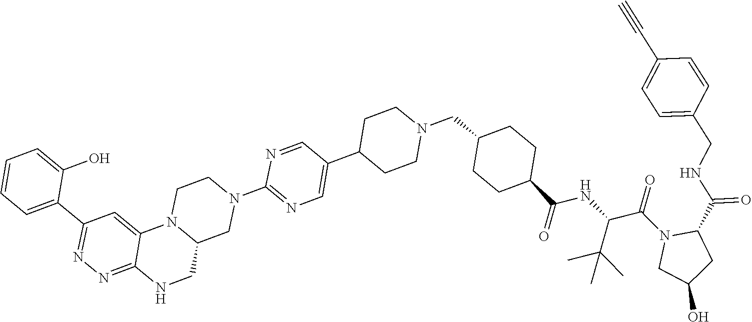 [SMILES: C#Cc1ccc(CNC(=O)[C@@H]2C[C@@H](O)CN2C(=O)[C@@H](NC(=O)[C@H]2CC[C@H](CN3CCC(c4cnc(N5CCN6c7cc(-c8ccccc8O)nnc7NC[C@H]6C5)nc4)CC3)CC2)C(C)(C)C)cc1] |
| I-296 | 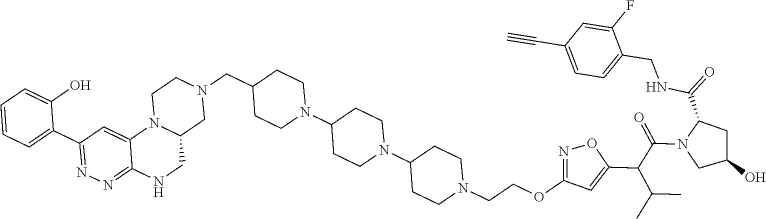 [SMILES: C#Cc1ccc(CNC(=O)[C@@H]2C[C@@H](O)CN2C(=O)C(c2cc(OCCN3CCC(N4CCC(N5CCC(CN6CCN7c8cc(-c9ccccc9O)nnc8NC[C@H]7C6)CC5)CC4)CC3)no2)C(C)C)c(F)c1] |
| I-298 | 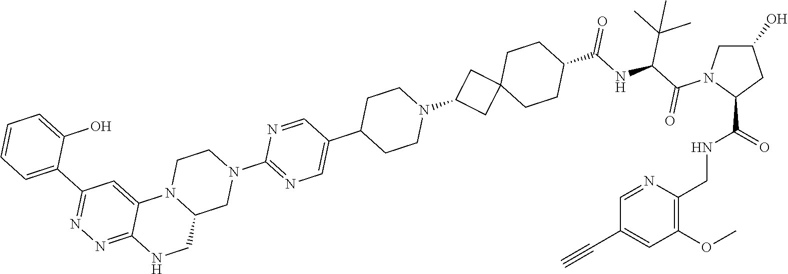 [SMILES: C#Cc1cnc(CNC(=O)[C@@H]2C[C@@H](O)CN2C(=O)[C@@H](NC(=O)[C@H]2CCC3(CC2)C[C@H](N2CCC(c4cnc(N5CCN6c7cc(-c8ccccc8O)nnc7NC[C@H]6C5)nc4)CC2)C3)C(C)(C)C)c(OC)c1] |
| I-299 | 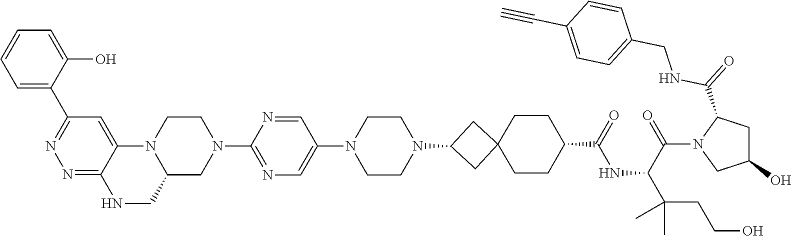 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)CCO)cc1] |
| I-300 | 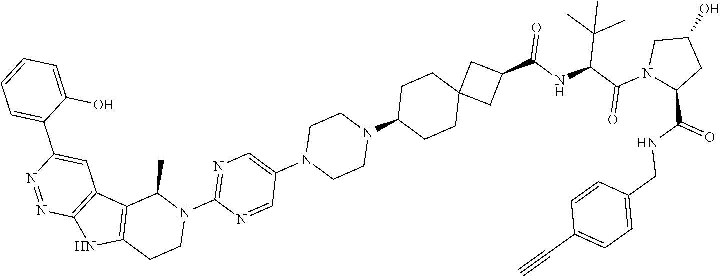 [SMILES: C#Cc1ccc(CNC(=O)[C@@H]2C[C@@H](O)CN2C(=O)[C@@H](NC(=O)[C@H]2CC3(CC[C@H](N4CCN(c5cnc(N6CCc7[nH]c8nnc(-c9ccccc9O)cc8c7[C@H]6C)nc5)CC4)CC3)C2)C(C)(C)C)cc1] |
| I-301 | 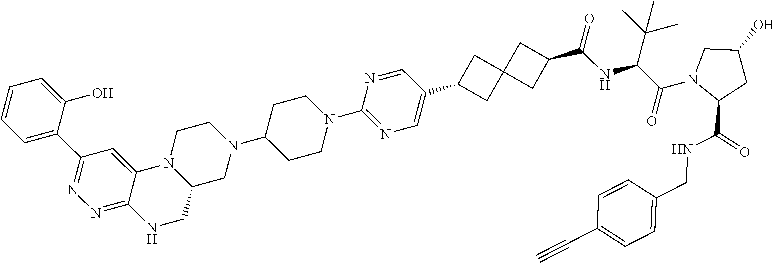 [SMILES: C#Cc1ccc(CNC(=O)[C@@H]2C[C@@H](O)CN2C(=O)[C@@H](NC(=O)[C@H]2CC3(C2)C[C@H](c2cnc(N4CCC(N5CCN6c7cc(-c8ccccc8O)nnc7NC[C@H]6C5)CC4)nc2)C3)C(C)(C)C)cc1] |
| I-302 | 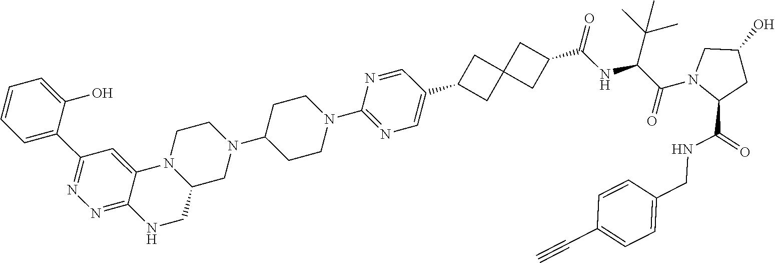 [SMILES: C#Cc1ccc(CNC(=O)[C@@H]2C[C@@H](O)CN2C(=O)[C@@H](NC(=O)[C@H]2CC3(C2)C[C@H](c2cnc(N4CCC(N5CCN6c7cc(-c8ccccc8O)nnc7NC[C@H]6C5)CC4)nc2)C3)C(C)(C)C)cc1] |
| I-303 | 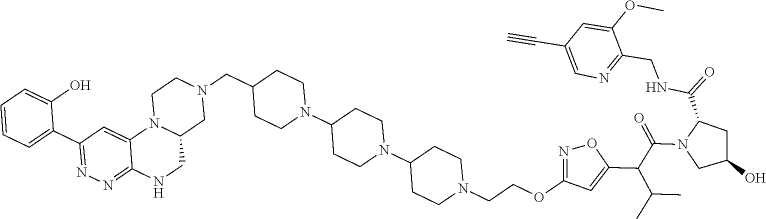 [SMILES: C#Cc1cnc(CNC(=O)[C@@H]2C[C@@H](O)CN2C(=O)C(c2cc(OCCN3CCC(N4CCC(N5CCC(CN6CCN7c8cc(-c9ccccc9O)nnc8NC[C@H]7C6)CC5)CC4)CC3)no2)C(C)C)c(OC)c1] |
| I-304 | 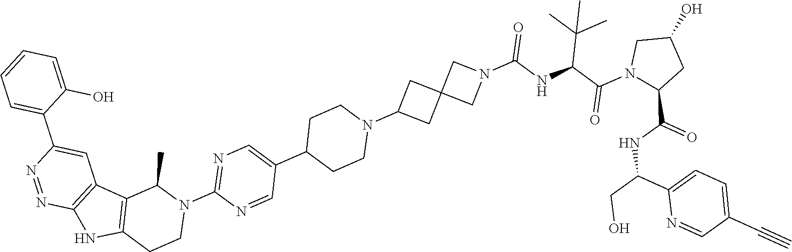 [SMILES: C#Cc1ccc([C@H](CO)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)nc1] |
| I-305 | 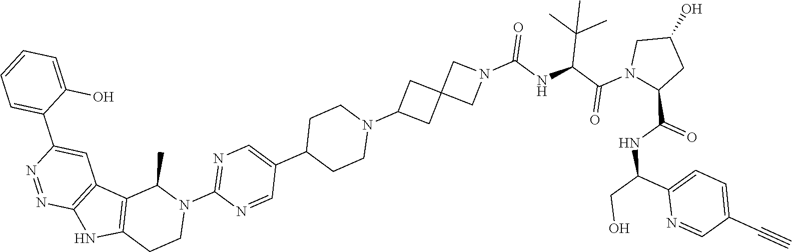 [SMILES: C#Cc1ccc([C@@H](CO)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)nc1] |
| I-306 | 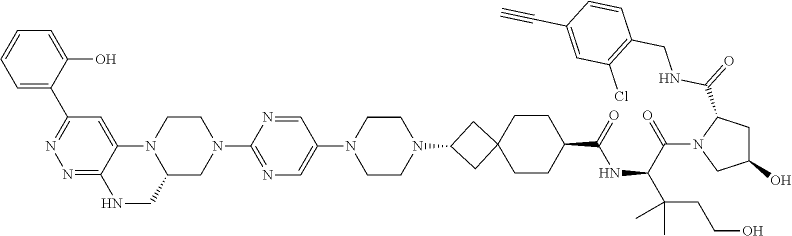 [SMILES: C#Cc1ccc(CNC(=O)[C@@H]2C[C@@H](O)CN2C(=O)[C@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)CCO)c(Cl)c1] |
| I-307 | 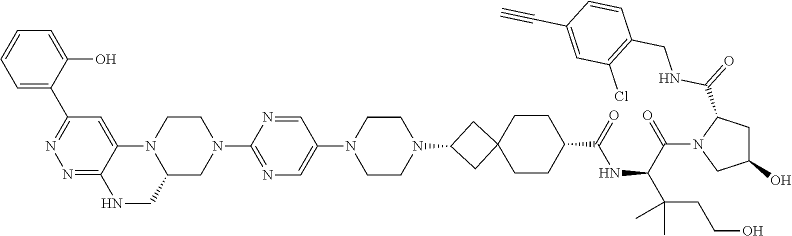 [SMILES: C#Cc1ccc(CNC(=O)[C@@H]2C[C@@H](O)CN2C(=O)[C@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)CCO)c(Cl)c1] |
| I-309 | 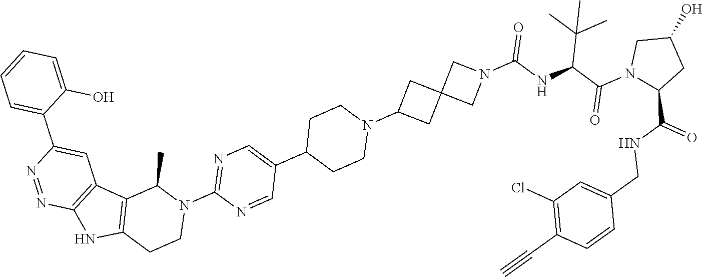 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1Cl] |
| I-310 | 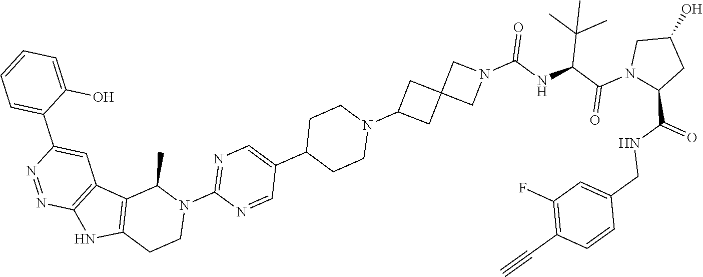 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1F] |
| I-311 | 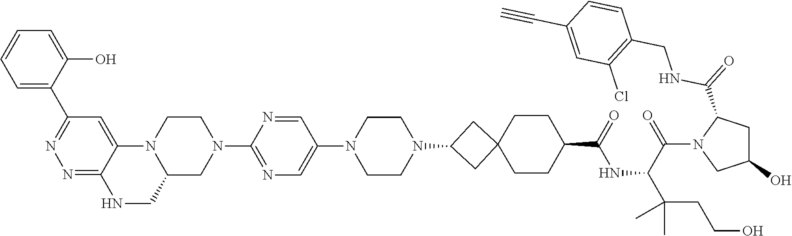 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)CCO)c(Cl)c1] |
| I-312 | 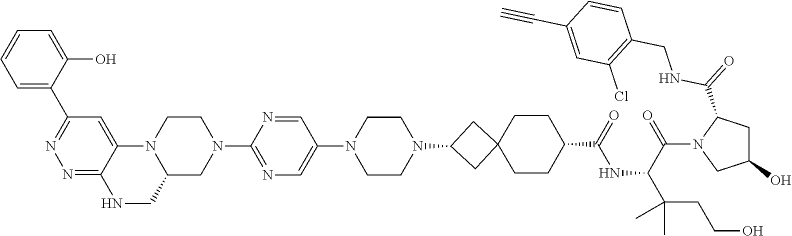 [SMILES: C#Cc1ccc(CNC(=O)[C@@H]2C[C@@H](O)CN2C(=O)[C@@H](NC(=O)[C@H]2CCC3(CC2)C[C@H](N2CCN(c4cnc(N5CCN6c7cc(-c8ccccc8O)nnc7NC[C@H]6C5)nc4)CC2)C3)C(C)(C)CCO)c(Cl)c1] |
| I-316 | 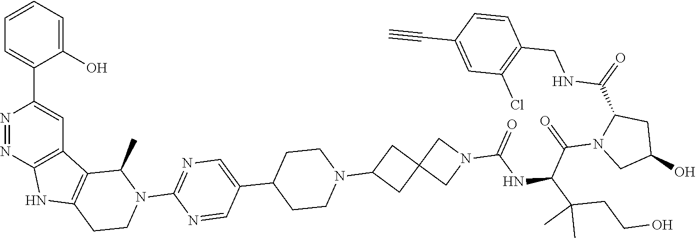 [SMILES: C#Cc1ccc(CNC(=O)[C@@H]2C[C@@H](O)CN2C(=O)[C@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCO)c(Cl)c1] |
| I-317 | 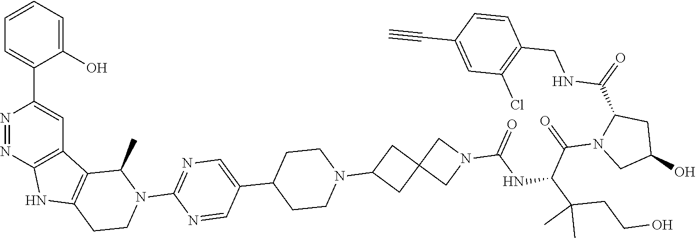 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCO)c(Cl)c1] |
| I-318 | 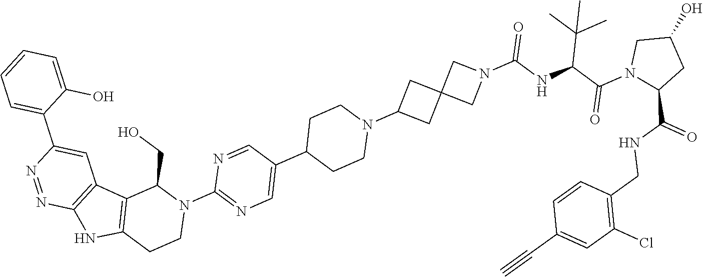 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6CO)nc5)CC4)C3)C2)C(C)(C)C)c(Cl)c1] |
| I-319 | 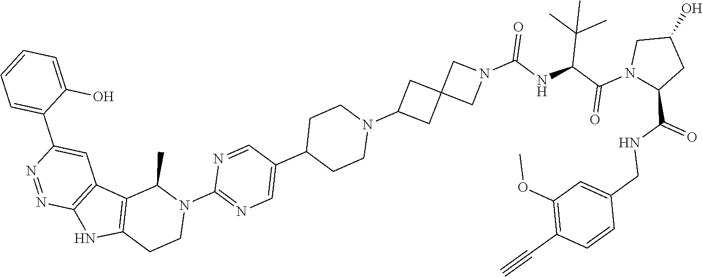 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1OC] |
| I-320 | 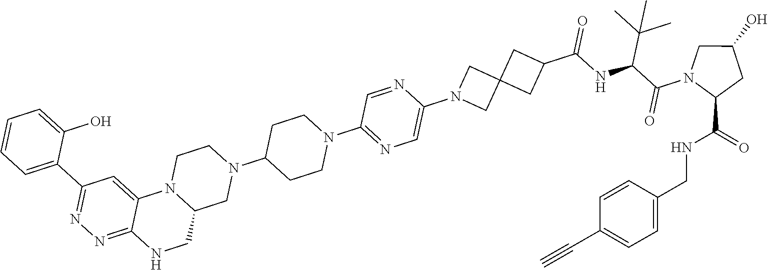 [SMILES: C#Cc1ccc(CNC(=O)[C@@H]2C[C@@H](O)CN2C(=O)[C@@H](NC(=O)C2CC3(C2)CN(c2cnc(N4CCC(N5CCN6c7cc(-c8ccccc8O)nnc7NC[C@H]6C5)CC4)cn2)C3)C(C)(C)C)cc1] |
| I-321 | 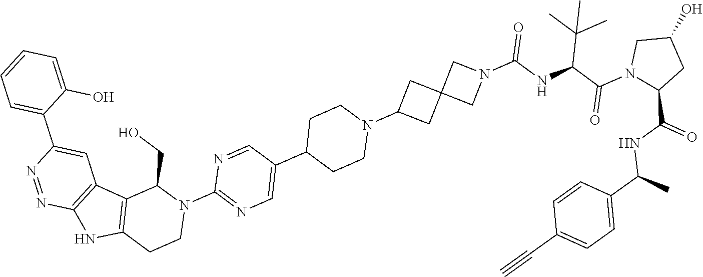 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6CO)nc5)CC4)C3)C2)C(C)(C)C)cc1] |
| I-323 | 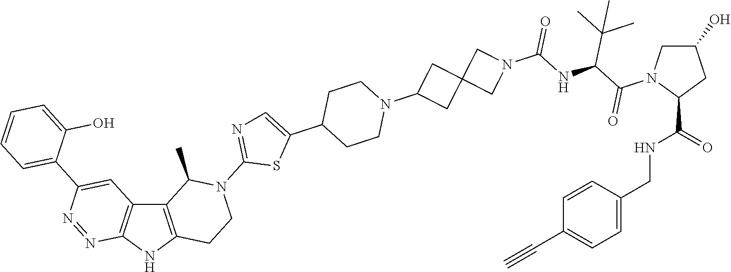 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)s5)CC4)C3)C2)C(C)(C)C)cc1] |
| I-324 | 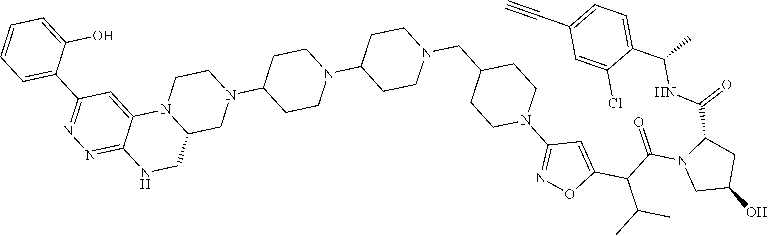 [SMILES: C#Cc1ccc([C@H](C)NC(=O)[C@@H]2C[C@@H](O)CN2C(=O)C(c2cc(N3CCC(CN4CCC(N5CCC(N6CCN7c8cc(-c9ccccc9O)nnc8NC[C@H]7C6)CC5)CC4)CC3)no2)C(C)C)c(Cl)c1] |
| I-325 | 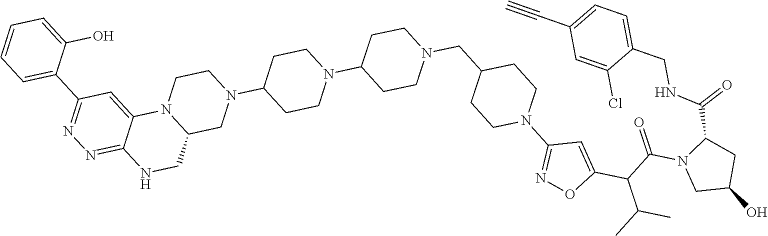 [SMILES: C#Cc1ccc(CNC(=O)[C@@H]2C[C@@H](O)CN2C(=O)C(c2cc(N3CCC(CN4CCC(N5CCC(N6CCN7c8cc(-c9ccccc9O)nnc8NC[C@H]7C6)CC5)CC4)CC3)no2)C(C)C)c(Cl)c1] |
| I-327 | 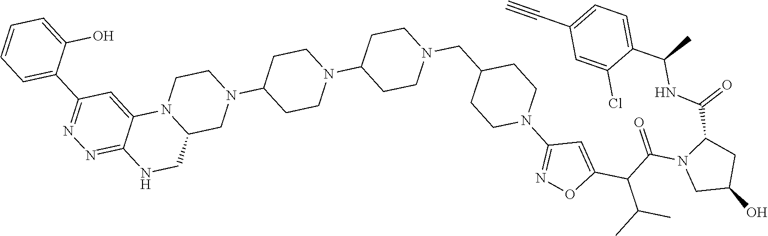 [SMILES: C#Cc1ccc([C@@H](C)NC(=O)[C@@H]2C[C@@H](O)CN2C(=O)C(c2cc(N3CCC(CN4CCC(N5CCC(N6CCN7c8cc(-c9ccccc9O)nnc8NC[C@H]7C6)CC5)CC4)CC3)no2)C(C)C)c(Cl)c1] |
| I-328 | 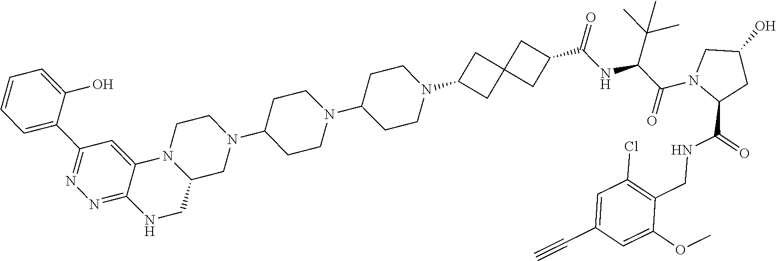 [SMILES: C#Cc1cc(Cl)c(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(OC)c1] |
| I-329 | 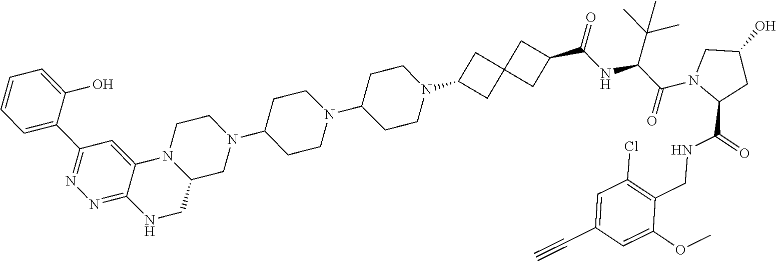 [SMILES: C#Cc1cc(Cl)c(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(OC)c1] |
| I-330 | 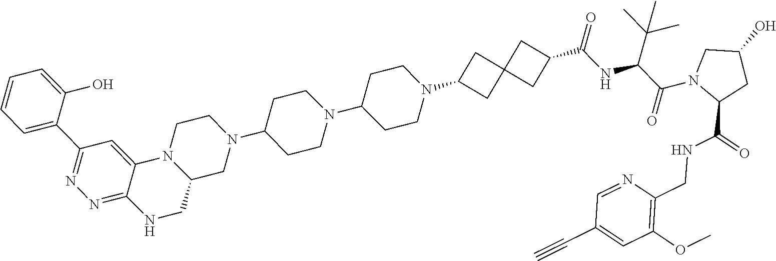 [SMILES: C#Cc1cnc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(OC)c1] |
| I-331 | 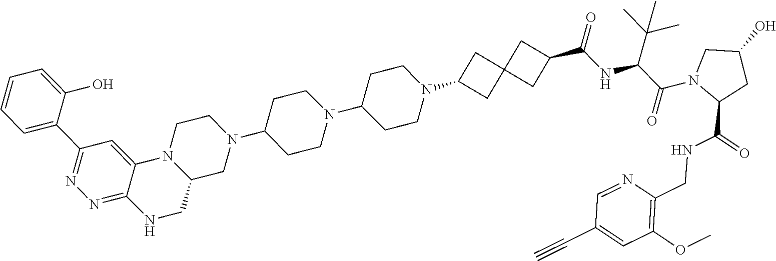 [SMILES: C#Cc1cnc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)C)c(OC)c1] |
| I-332 | 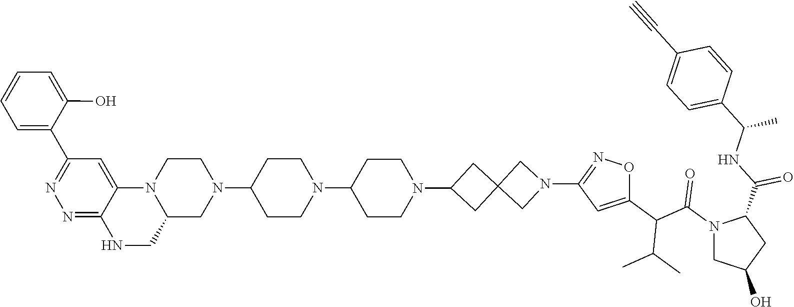 [SMILES: C#Cc1ccc([C@H](C)NC(=O)[C@@H]2C[C@@H](O)CN2C(=O)C(c2cc(N3CC4(CC(N5CCC(N6CCC(N7CCN8c9cc(-c%10ccccc%10O)nnc9NC[C@H]8C7)CC6)CC5)C4)C3)no2)C(C)C)cc1] |
| I-333 | 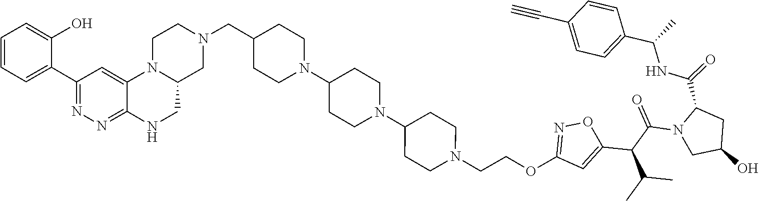 [SMILES: C#Cc1ccc([C@H](C)NC(=O)[C@@H]2C[C@@H](O)CN2C(=O)[C@H](c2cc(OCCN3CCC(N4CCC(N5CCC(CN6CCN7c8cc(-c9ccccc9O)nnc8NC[C@H]7C6)CC5)CC4)CC3)no2)C(C)C)cc1] |
| I-334 | 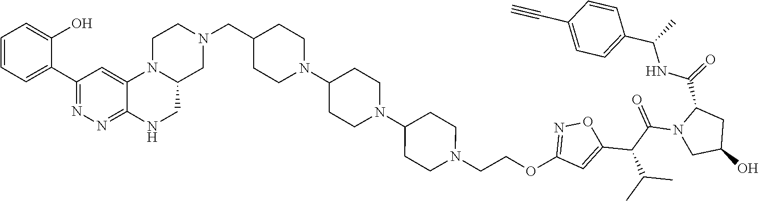 [SMILES: C#Cc1ccc([C@H](C)NC(=O)[C@@H]2C[C@@H](O)CN2C(=O)[C@@H](c2cc(OCCN3CCC(N4CCC(N5CCC(CN6CCN7c8cc(-c9ccccc9O)nnc8NC[C@H]7C6)CC5)CC4)CC3)no2)C(C)C)cc1] |
| I-335 | 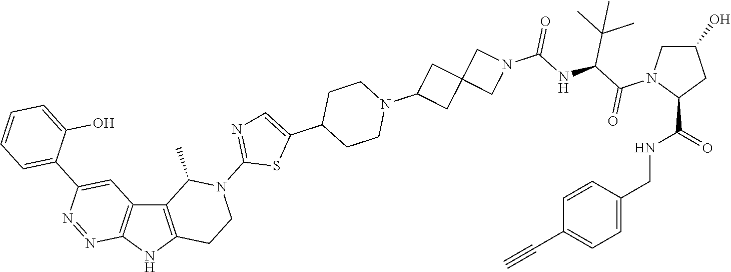 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@@H]6C)s5)CC4)C3)C2)C(C)(C)C)cc1] |
| I-337 | 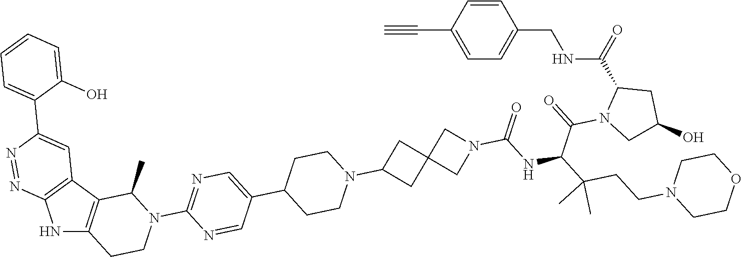 [SMILES: C#Cc1ccc(CNC(=O)[C@@H]2C[C@@H](O)CN2C(=O)[C@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCN2CCOCC2)cc1] |
| I-338 | 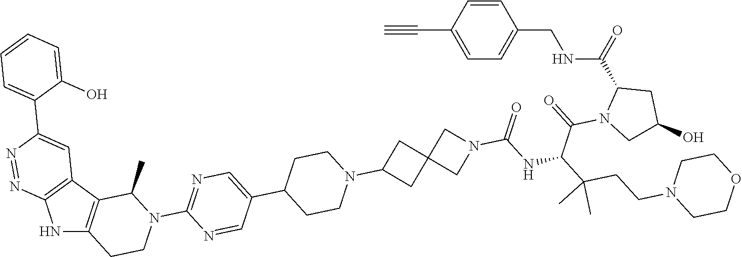 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCN2CCOCC2)cc1] |
| I-339 | 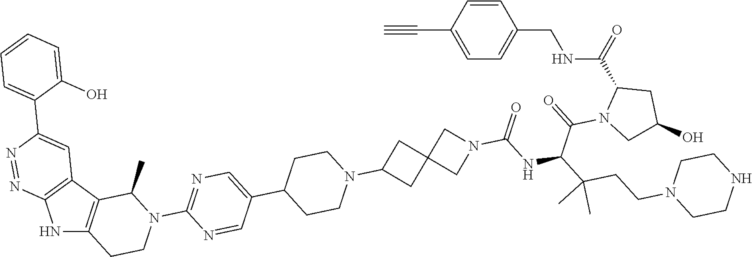 [SMILES: C#Cc1ccc(CNC(=O)[C@@H]2C[C@@H](O)CN2C(=O)[C@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCN2CCNCC2)cc1] |
| I-340 | 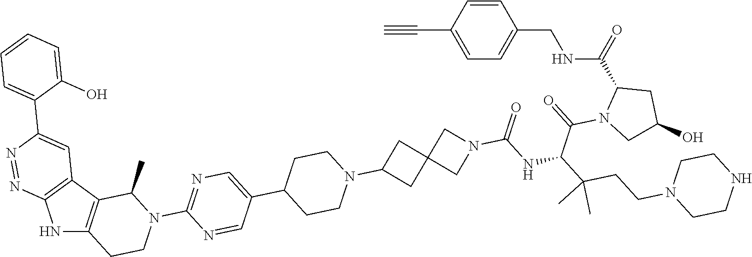 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCN2CCNCC2)cc1] |
| I-341 | 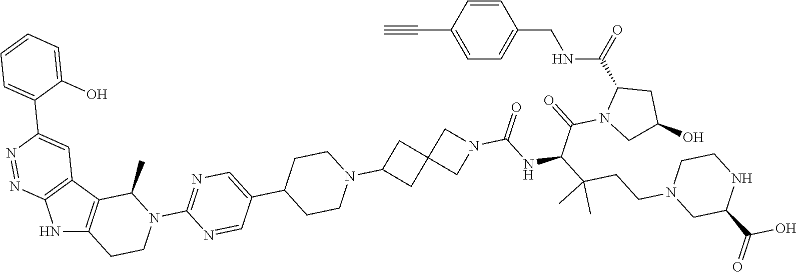 [SMILES: C#Cc1ccc(CNC(=O)[C@@H]2C[C@@H](O)CN2C(=O)[C@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCN2CCN[C@@H](C(=O)O)C2)cc1] |
| I-342 | 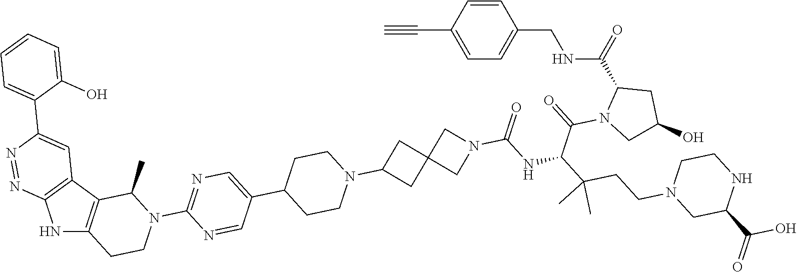 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCN2CCN[C@@H](C(=O)O)C2)cc1] |
| I-343 | 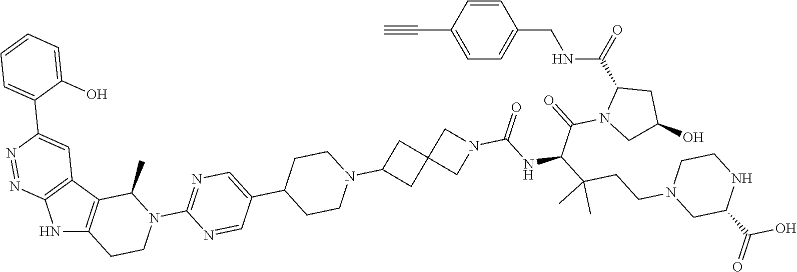 [SMILES: C#Cc1ccc(CNC(=O)[C@@H]2C[C@@H](O)CN2C(=O)[C@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCN2CCN[C@H](C(=O)O)C2)cc1] |
| I-344 | 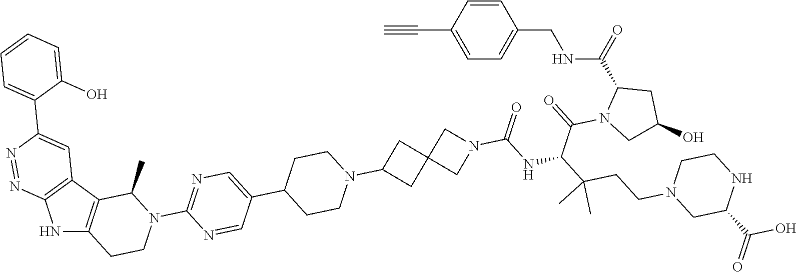 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)CCN2CCN[C@H](C(=O)O)C2)cc1] |
| I-362 | 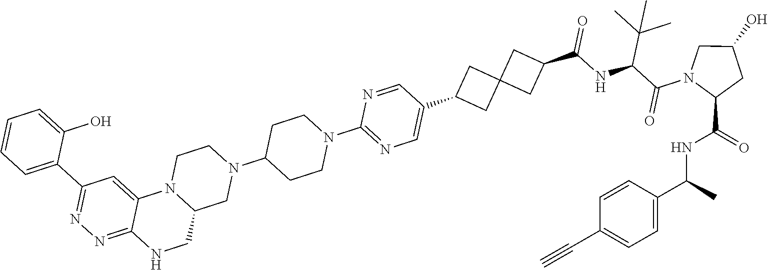 [SMILES: C#Cc1ccc([C@H](C)NC(=O)[C@@H]2C[C@@H](O)CN2C(=O)[C@@H](NC(=O)[C@H]2CC3(C2)C[C@H](c2cnc(N4CCC(N5CCN6c7cc(-c8ccccc8O)nnc7NC[C@H]6C5)CC4)nc2)C3)C(C)(C)C)cc1] |
| I-363 | 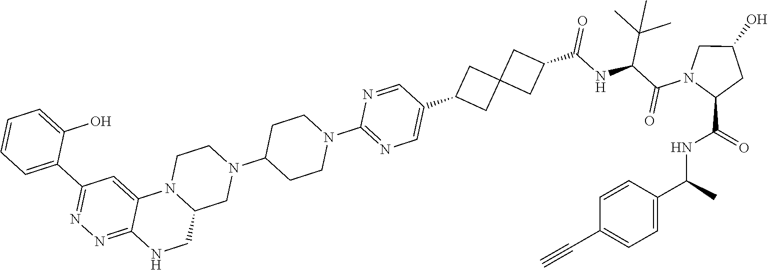 [SMILES: C#Cc1ccc([C@H](C)NC(=O)[C@@H]2C[C@@H](O)CN2C(=O)[C@@H](NC(=O)[C@H]2CC3(C2)C[C@H](c2cnc(N4CCC(N5CCN6c7cc(-c8ccccc8O)nnc7NC[C@H]6C5)CC4)nc2)C3)C(C)(C)C)cc1] |
| I-364 | 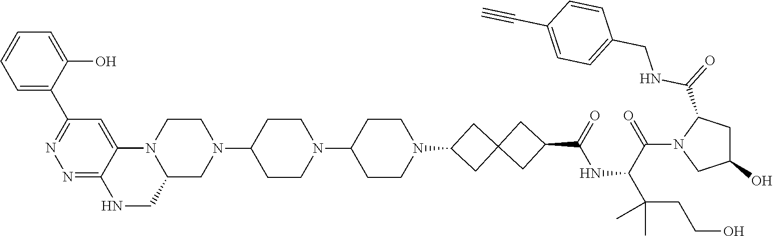 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)CCO)cc1] |
| I-365 | 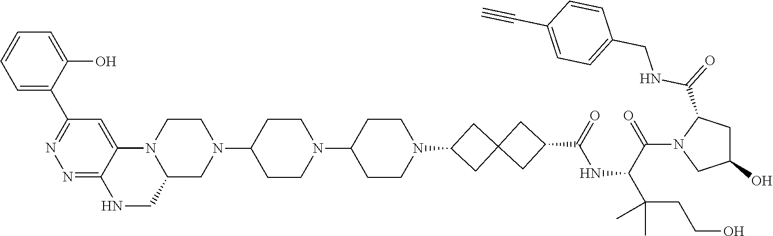 [SMILES: C#Cc1ccc(CNC(=O)[C@@H]2C[C@@H](O)CN2C(=O)[C@@H](NC(=O)[C@H]2CC3(C2)C[C@H](N2CCC(N4CCC(N5CCN6c7cc(-c8ccccc8O)nnc7NC[C@H]6C5)CC4)CC2)C3)C(C)(C)CCO)cc1] |
| I-369 | 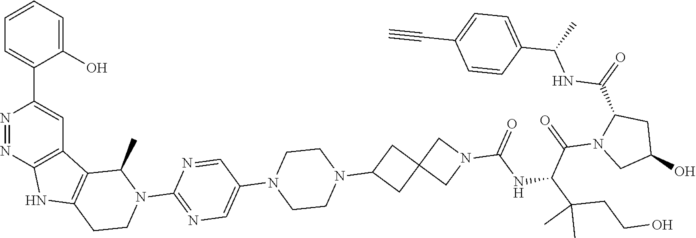 [SMILES: C#Cc1ccc([C@H](C)NC(=O)[C@@H]2C[C@@H](O)CN2C(=O)[C@@H](NC(=O)N2CC3(CC(N4CCN(c5cnc(N6CCc7[nH]c8nnc(-c9ccccc9O)cc8c7[C@H]6C)nc5)CC4)C3)C2)C(C)(C)CCO)cc1] |
| I-402 | 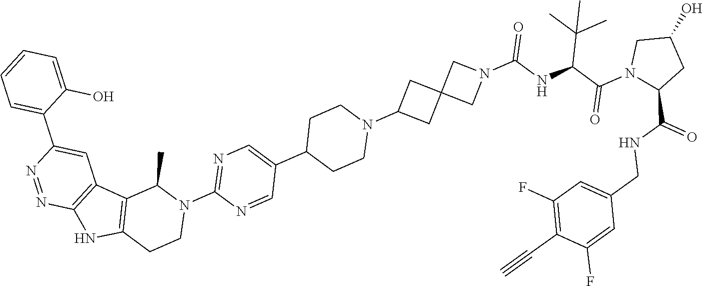 [SMILES: C#Cc1c(F)cc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1F] |
| I-403 | 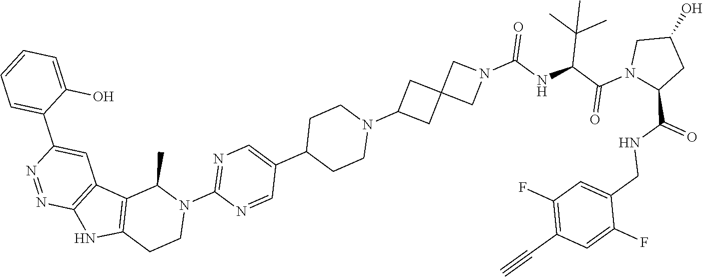 [SMILES: C#Cc1cc(F)c(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1F] |
| I-404 | 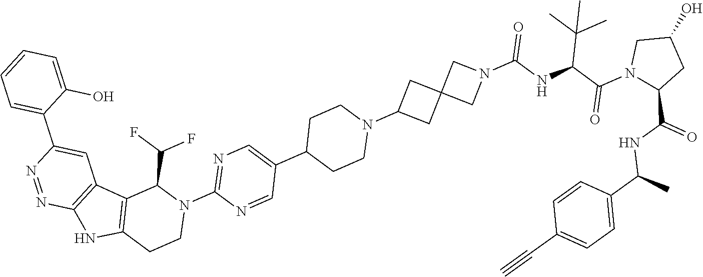 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C(F)F)nc5)CC4)C3)C2)C(C)(C)C)cc1] |
| I-405 | 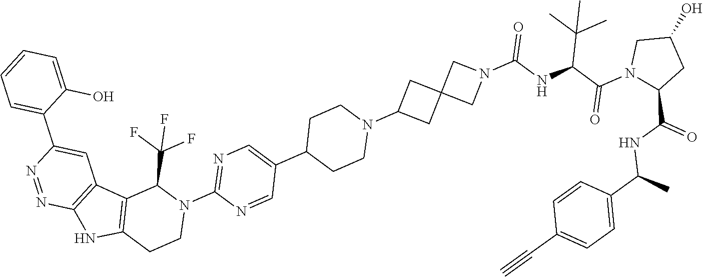 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C(F)(F)F)nc5)CC4)C3)C2)C(C)(C)C)cc1] |
| I-410 | 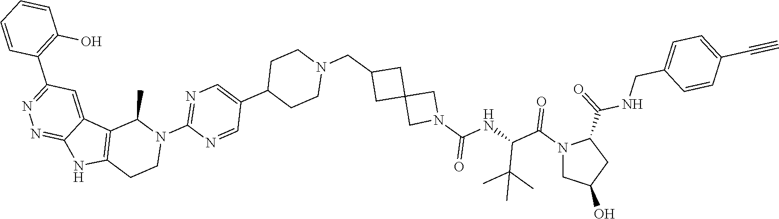 [SMILES: C#Cc1ccc(CNC(=O)[C@@H]2C[C@@H](O)CN2C(=O)[C@@H](NC(=O)N2CC3(CC(CN4CCC(c5cnc(N6CCc7[nH]c8nnc(-c9ccccc9O)cc8c7[C@H]6C)nc5)CC4)C3)C2)C(C)(C)C)cc1] |
| I-411 | 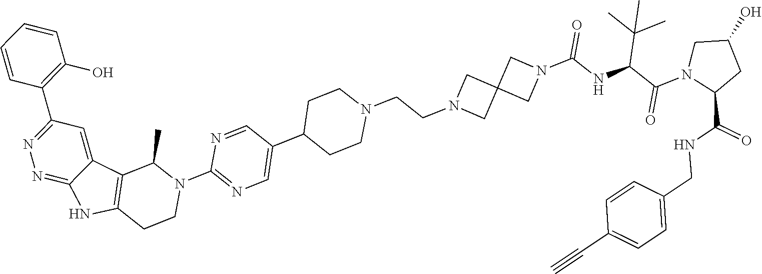 [SMILES: C#Cc1ccc(CNC(=O)[C@@H]2C[C@@H](O)CN2C(=O)[C@@H](NC(=O)N2CC3(CN(CCN4CCC(c5cnc(N6CCc7[nH]c8nnc(-c9ccccc9O)cc8c7[C@H]6C)nc5)CC4)C3)C2)C(C)(C)C)cc1] |
| I-413 | 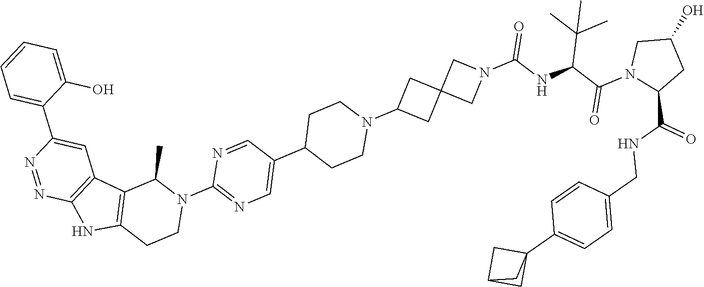 [SMILES: C[C@@H]1c2c([nH]c3nnc(-c4ccccc4O)cc23)CCN1c1ncc(C2CCN(C3CC4(C3)CN(C(=O)N[C@H](C(=O)N3C[C@H](O)C[C@H]3C(=O)NCc3ccc(C56CC(C5)C6)cc3)C(C)(C)C)C4)CC2)cn1] |
| I-416 | 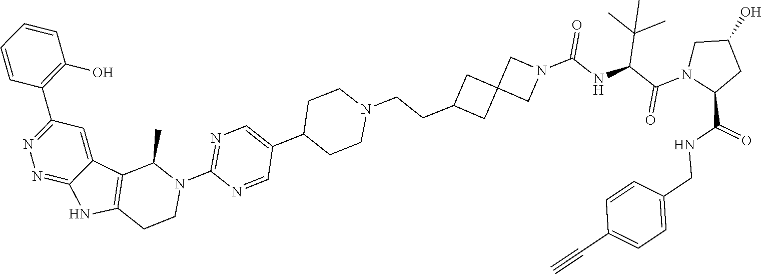 [SMILES: C#Cc1ccc(CNC(=O)[C@@H]2C[C@@H](O)CN2C(=O)[C@@H](NC(=O)N2CC3(CC(CCN4CCC(c5cnc(N6CCc7[nH]c8nnc(-c9ccccc9O)cc8c7[C@H]6C)nc5)CC4)C3)C2)C(C)(C)C)cc1] |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3′^{d} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.
Amino protecting groups are well known in the art and include those described in detail in Protecting Groups in Organic Synthesis, T. W. Greene and P. G. M. Wuts, 3rd edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a final degrader is formed having a reactive LBM moiety (e.g., amine, alcohol, etc.), it is not shown but it is generally appreciated and well known by those having ordinary skill in the art that the reactivity of said reactive LBM moiety may be masked by employing a suitable protecting group that can thereafter be removed in situ or during a separate synthetic step to form the final degrader product. In the below schemes, DIM=LBM.

In certain embodiments, compounds of the present invention are generally prepared according to Scheme 1 set forth below.

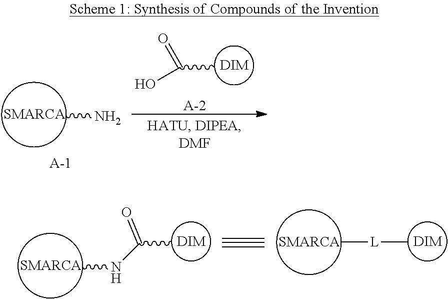

As depicted in Scheme 1, above, amine A-1 is coupled to acid A-2 using the coupling agent HATU in the presence of the base DIPEA in DMF to form a compound of the invention with a linker comprising an amide bond. The squiggly bond, [symbol] , represents the portion of the linker between SMARCA and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 2 set forth below:

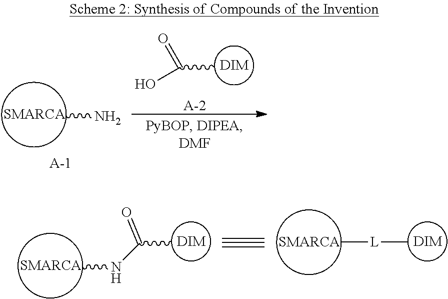

As depicted in Scheme 2, above, amine A-1 is coupled to acid A-2 using the coupling agent PyBOP in the presence of the base DIPEA in DMF to form a compound of the invention with a linker comprising an amide bond. The squiggly bond, [symbol] , represents the portion of the linker between SMARCA and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 3 set forth below:

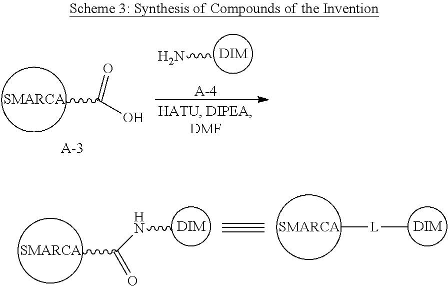

As depicted in Scheme 3, above, acid A-3 is coupled to amine A-4 using the coupling agent HATU in the presence of the base DIPEA in DMF to form a compound of the invention with a linker comprising an amide bond. The squiggly bond, [symbol] , represents the portion of the linker between SMARCA and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 4 set forth below:

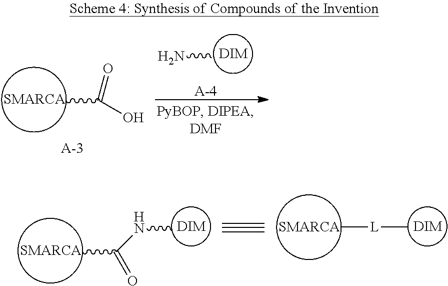

As depicted in Scheme 4, above, acid A-3 is coupled to amine A-4 using the coupling agent PyBOP in the presence of the base DIPEA in DMF to form a compound of the invention with a linker comprising an amide bond. The squiggly bond, [symbol] , represents the portion of the linker between SMARCA and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 5 set forth below:

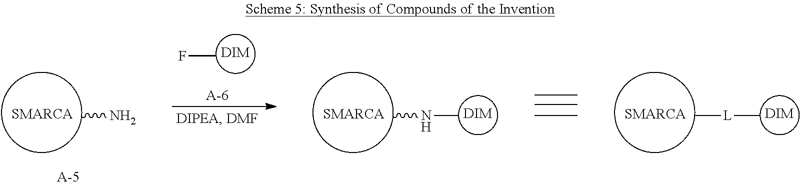

As depicted in Scheme 5, above, an S_{N}Ar displacement of fluoride A-6 by amine A-5 is effected in the presence of the base DIPEA in DMF to form a compound of the invention with a linker comprising a secondary amine. The squiggly bond, [symbol] , represents the portion of the linker between SMARCA and the terminal amino group of A-5.

In certain embodiments, compounds of the present invention are generally prepared according to Scheme 6 set forth below:

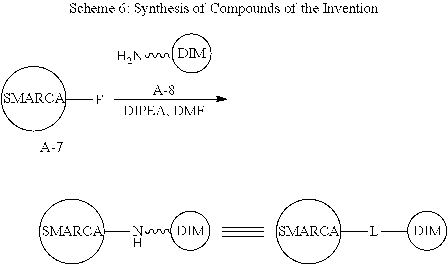

As depicted in Scheme 6, above, an S_{N}Ar displacement of fluoride A-7 by amine A-8 is effected in the presence of the base DIPEA in DMF to form a compound of the invention with a linker comprising a secondary amine. The squiggly bond, [symbol] , represents the portion of the linker between DIM and the terminal amino group of A-8.

In certain embodiments, compounds of the present invention are generally prepared according to Scheme 7 set forth below:

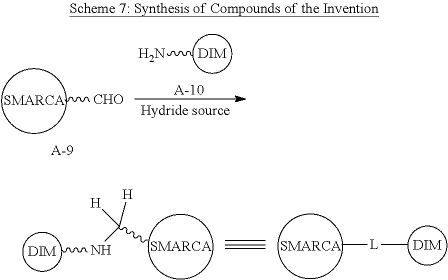

As depicted in Scheme 7, above, reductive alkylation of aldehyde A-9 by amine A-10 is effected in the presence of a mild hydride source (e.g., sodium cyanoborohydride or sodium triacetoxyborohydride) to form a provided compound with a linker comprising a secondary amine. The squiggly bond, [symbol] , represents the portion of the linker between DIM and the terminal amino group of A-10.

In certain embodiments, compounds of the present invention are generally prepared according to Scheme 8 set forth below:

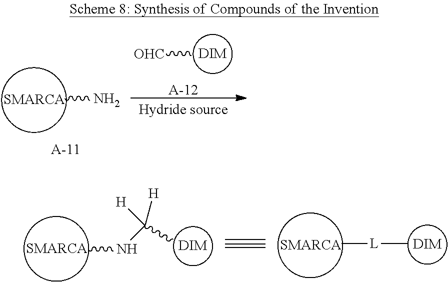

As depicted in Scheme 8, above, reductive alkylation of aldehyde A-12 by amine A-11 is effected in the presence of a mild hydride source (e.g., sodium cyanoborohydride or sodium triacetoxyborohydride) to form a provided compound with a linker comprising a secondary amine. The squiggly bond, [symbol] , represents the portion of the linker between SMARCA and the terminal amino group of A-11.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, “March's Advanced Organic Chemistry”, 5^{th} Ed., Ed.: Smith, M. B. and March, J., John Wiley & Sons, New York: 2001, the entirety of each of which is herein incorporated by reference. Such interconversions may require one or more of the aforementioned techniques, and certain methods for synthesizing compounds of the invention are described below in the Exemplification.

### 5. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit a SMARCA and/or PB1 protein, or a mutant thereof, in a biological sample or in a patient. In certain embodiments, the amount of compound in compositions of this invention is such that is effective to measurably degrade and/or inhibit a SMARCA and/or PB1 protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of a SMARCA and/or PB1 protein, or a mutant thereof.

As used herein, the term “degratorily active metabolite or residue thereof” means that a metabolite or residue thereof is also a degrader of an SMARCA and/or PB1 protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition. In some embodiments, a provided compound is administered (e.g., intravenously) to a patient intermittently (e.g., weekly).

Uses of Compounds and Pharmaceutically Acceptable Compositions

Compounds and compositions described herein are generally useful for the degradation and/or inhibition of a SMARCA or PB1 protein activity.

Examples of SMARCA proteins that are degraded and/or inhibited by the compounds and compositions described herein and against which the methods described herein are useful include those of the SWI/SNF-related matrix-associated actin-dependent regulators of chromatin subfamily A (“SMARCA”) family of proteins, the members of which include SMARCA1, SMARCA2, SMARCA4, or SMARCA5, or a mutant thereof. See e.g., Shain and Pollack “The Spectrum of SWI/SNF Mutations, Ubiquitous in Human Cancers. PLoS One 2013, 8:e55119; Kadoch and Crabtree “Mammalian SWI/SNF Chromatin Remodeling Complexes and Cancer: Mechanistic Insights Gained from Human Genomics” Sci. Adv. 2015, 1:e1500447; Wilson and Roberts “SWI/SNF Nucleosome Remodelers and Cancer” Nat. Rev. Cancer 2011, 11:481; and Son and Crabtree “The Role of BAF (mSWI/SNF) Complexes in Mammalian Neural Development” Am. J. Med. Genet., Part C 2014, 166:333, the entirety of each of which is herein incorporated by reference.

The activity of a compound utilized in this invention as a degrader and/or inhibitor of one or more SMARCA or PB1, or a mutant thereof, may be assayed in vitro, in vivo or in a cell line. In vitro assays include assays that determine inhibition of either the activity and/or the subsequent functional consequences of activated SMARCA or PB1 protein, or a mutant thereof. Alternate in vitro assays quantitate the ability of the inhibitor to bind to a SMARCA or PB1 protein. Inhibitor binding may be measured by radiolabeling the inhibitor prior to binding, isolating the inhibitor/SMARCA or PB1 complex and determining the amount of radiolabel bound. Alternatively, inhibitor binding may be determined by running a competition experiment where new inhibitors are incubated with a SMARCA or PB1 protein bound to known radioligands. Representative in vitro and in vivo assays useful in assaying a SMARCA or PB1 inhibitor include those described and disclosed in, e.g., Tanaka et al. “Design and Characterization of Bivalent BET Inhibitors” Nat. Chem. Biol. 2016, 12(12):1089; Schiaffino-Ortega et al. “SWI/SNF as targets in cancer therapy” J. Hematol. Oncol. 2014, 7:81; Filippakopoulos et al. “Histone Recognition and Large-Scale Structural Analysis of the Human Bromodomain Family” Cell 2012, 149:214. Detailed conditions for assaying a compound utilized in this invention as a degrader and/or inhibitor of a SMARCA or PB1 protein, or a mutant thereof, are set forth in the Examples below.

Chromatin is a complex combination of DNA and protein that makes up chromosomes. Chromatin functions to package, strengthen, and control expression and DNA replication. The chromatin structure is controlled by a series of post-translational modifications, most commonly within the “histone tails” which extend beyond the core nucleosome structure. These epigenetic modifications including acetylation, methylation, phosphorylation, ubiquitinylation, and SUMOylation, is then interpreted by the cell to allow gene specific regulation of chromatin structure and thereby transcription. Histone modifications are dynamic, as they can be added or removed in response to specific stimuli, and these modifications direct both structural changes to chromatin and alterations in gene transcription. Distinct classes of enzymes, namely histone acetyltransferases (HATs) and histone deacetylases (HDACs), acetylate or de-acetylate specific histone lysine residues (Struhl, Genes Dev. 1989, 12(5):599).

Bromodomains, which are approximately 110 amino acids long, are found in a large number of chromatin-associated proteins and have been identified in approximately 70 human proteins, often adjacent to other protein motifs (Jeanmougin et al., Trends Biochem. Sci. 1997, 22(5):151; Tamkun et al., Cell 1992, 7(3):561). Interactions between bromodomains and modified histones may be an important mechanism underlying chromatin structural changes and gene regulation. Bromodomain-containing proteins have been implicated in disease processes including cancer, inflammation and viral replication. See, e.g., Prinjha et al, Trends Pharm. Sci. 2012, 33(3):146; Muller et al. Expert Rev. 2011, 13(29):1.

Cell-type specificity and proper tissue functionality requires the tight control of distinct transcriptional programs that are intimately influenced by their environment. Alterations to this transcriptional homeostasis are directly associated with numerous disease states, most notably cancer, immuno-inflammation, neurological disorders, and metabolic diseases. Bromodomains reside within key chromatin modifying complexes that serve to control distinctive disease-associated transcriptional pathways. An example of such a complex is the switch/sucrose nonfermenting (“SWI/SNF”) chromatin-remodeling complex, which has been reported to be involved in gene regulation, cell lineage specification and development, and comprises a number of bromodomain containing subunits, including SWI/SNF-related matrix-associated actin-dependent regulator of chromatin subfamily A member 2 and 4 (SMARCA2 and SMARCA4) and polybromo-1 (PB1; also known as PBRM1). SMARCA2 and SMARCA4, also known as transcription activators Brahma homologue (BRM) and Brahma-related gene 1 (BRG1) respectively, are mutually exclusive helicase/ATPase proteins of the large ATP-dependent SWI/SNF chromatin-remodeling complexes involved in transcriptional regulation of gene expression. In some embodiments, a provided compound binds to one or more SMARCA2, SMARCA4, or PB1 bromodomains. In some embodiments, a provided compound binds to one or more SMARCA2, SMARCA4, or PB1 ATPase domains.

Representative SMARCA2, SMARCA4, and/or PB1 inhibitors include those described and disclosed in e.g., Gerstenberger et al. J. Med. Chem. 2016, 59(10):4800; Theodoulou et al. Curr Opin. Chem. Bio. 2016, 33:58; Vangamudi et al. Cancer Res. 2015, 75(18):3865; the entirety of each of which is herein incorporated by reference.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are degraders and/or inhibitors of one of more SMARCA2, SMARCA4, or PB1 protein and are therefore useful for treating one or more disorders associated with activity of one or more of SMARCA2, SMARCA4, or PB1 protein. Thus, in certain embodiments, the present invention provides a method for treating a SMARCA2-mediated, SMARCA4-mediated, or PB1-mediated disorder comprising the step of administering to a patient in need thereof a compound of the present invention, or pharmaceutically acceptable composition thereof.

As used herein, the terms “SMARCA2-mediated”, “SMARCA4-mediated”, or “PB1-mediated” disorders, diseases, and/or conditions as used herein means any disease or other deleterious condition in which one or more SMARCA2, SMARCA4, or PB1, or a mutant thereof, are known to play a role. Accordingly, another embodiment of the present invention relates to treating or lessening the severity of one or more diseases in which one or more SMARCA2, SMARCA4, or PB1, or a mutant thereof, are known to play a role.

In some embodiments, the present invention provides a method for treating one or more disorders, diseases, and/or conditions wherein the disorder, disease, or condition is a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

Diseases and conditions treatable according to the methods of this invention include, but are not limited to, cancer (see, e.g., Schiaffino-Ortega et al. J. Hematol. Oncol. 2014, 7:81; Medina et al. Gene Chromosome Canc. 2014, 41:170), diabetes, cardiovascular disease (see, e.g., Bevilacqua et al., Cardiovasc. Pathol. 2013, 23(2):85), viral disease, autoimmune diseases such as lupus, and rheumatoid arthritis, autoinflammatory syndromes, atherosclerosis (see, e.g., Ortiz-Mao et al., J. Proteom Genom Res. 2017, 2(1):1), psoriasis, allergic disorders, inflammatory bowel disease, inflammation, acute and chronic gout and gouty arthritis, neurological disorders (see, e.g., Pandey et al., J. Hum. Genet. 2004, 49:596), metabolic syndrome, immunodeficiency disorders such as AIDS and HIV (see, e.g., Boehm et al., Viruses 2013, 5:1571), genetic disorders (see, e.g., Kosho et al., Am. J. Med. Genet. 2014, 166(3):262; Tang et al., Am. J. Med. Genet. 2015, 173(1):195), destructive bone disorders, osteoarthritis (see, e.g., Tian, J Orthop. Surg. Res. 2018, 13:49), proliferative disorders (see, e.g., Cruickshank et al., PLoS One 2015, 10(11):e0142806), Waldenstrom's Macroglobulinemia. infectious diseases, conditions associated with cell death, pathologic immune conditions involving T cell activation, and CNS disorders (see, e.g., Koga et al., Human Mol. Gen. 2009, 18(13):2483) in a patient. In one embodiment, a human patient is treated with a compound of the current invention and a pharmaceutically acceptable carrier, adjuvant, or vehicle, wherein said compound is present in an amount to measurably degrade and/or inhibit one or more SMARCA2, SMARCA4, or PB1, or a mutant thereof

Compounds of the current invention are useful in the treatment of a proliferative disease selected from a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkin's and Non-Hodgkin's, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In certain embodiments, the cancer treated by a provided compound is lung cancer, non-small cell lung cancer (NSCLC), small-cell lung cancer, glioma, breast cancer, pancreatic cancer, colorectal cancer, bladder cancer, endometrial cancer, penile cancer, esophagogastric cancer, hepatobiliary cancer soft tissue sarcoma, ovarian cancer, head and neck cancer, renal cell carcinoma, bone cancer, non-Hodgkin lymphoma, prostate cancer, embryonal tumors, germ cell tumors, cervical cancer, thyroid cancer, salivary gland cancer, gastrointestinal neuroendocrine tumor, uterine sarcoma, gastrointestinal stromal tumor, CNS cancer, thymic tumor, adrenocortical carcinoma, appendiceal cancer, small bowel cancer, non-melanoma skin cancer, and/or melanoma. In some embodiments, the cancer is lung cancer. In some embodiments, the lung cancer is NSCLC. In some embodiments, the cancer is breast cancer. In some embodiments, the cancer is melanoma.

In some embodiments, the present invention provides a method of treating lung cancer in a patient in need thereof, comprising administering a compound of the present invention, or a pharmaceutically acceptable salt thereof.

In some embodiments, the present invention provides a method of treating non-small cell lung cancer (NSCLC) in a patient in need thereof, comprising administering a compound of the present invention, or a pharmaceutically acceptable salt thereof.

In some embodiments, the present invention provides a method of treating glioma in a patient in need thereof, comprising administering a compound of the present invention, or a pharmaceutically acceptable salt thereof.

In some embodiments, the present invention provides a method of treating breast cancer in a patient in need thereof, comprising administering a compound of the present invention, or a pharmaceutically acceptable salt thereof.

In some embodiments, the present invention provides a method of treating pancreatic cancer in a patient in need thereof, comprising administering a compound of the present invention, or a pharmaceutically acceptable salt thereof.

In some embodiments, the present invention provides a method of treating colorectal cancer in a patient in need thereof, comprising administering a compound of the present invention, or a pharmaceutically acceptable salt thereof.

In some embodiments, the present invention provides a method of treating bladder cancer in a patient in need thereof, comprising administering a compound of the present invention, or a pharmaceutically acceptable salt thereof.

In some embodiments, the present invention provides a method of treating endometrial cancer in a patient in need thereof, comprising administering a compound of the present invention, or a pharmaceutically acceptable salt thereof.

In some embodiments, the present invention provides a method of treating penile cancer in a patient in need thereof, comprising administering a compound of the present invention, or a pharmaceutically acceptable salt thereof.

In some embodiments, the present invention provides a method of treating non-melanoma skin cancer in a patient in need thereof, comprising administering a compound of the present invention, or a pharmaceutically acceptable salt thereof.

In some embodiments, the present invention provides a method of treating melanoma in a patient in need thereof, comprising administering a compound of the present invention, or a pharmaceutically acceptable salt thereof.

SMARCA2 has recently been reported as a synthetic lethal target in SMARCA4-deficient cancers (e.g., cancers comprising SMARCA4 loss of function mutations and/or cancers having reduced or absent expression, e.g., due to epigenetic alterations). SMARCA2 depletion has been shown to selectively inhibit the growth of SMARCA4-mutant cancer cells (Hoffman et al., PNAS 2014, 111(8):3128; Oike et al., Cancer Res. 2013, 73(17):5508). In some embodiments, the cancer treated by aprovided compound is a SMARCA4-deficient cancer (e.g., a cancer harboring a loss of function mutation and/or having reduced or absent SMARCA4 expression).

It has also been shown that certain cancers are dependent on SMARCA4 for disease progression and are vulnerable to SMARCA4 inhibition, including certain acute leukemia and small cell lung cancers (Hohmann et al., Trends in Genetics, 2014, 30(8):356). In some embodiments, the cancer treated by a provided compound is leukemia (e.g., acute leukemia, e.g., acute myeloid leukemia), breast cancer, small cell lung cancer, or malignant rhabdoid tumors (MRT) (e.g., a SNF5-deficient malignant rhabdoid tumor).

In some embodiments, the present invention provides a method of treating leukemia in a patient in need thereof, comprising administering a compound of the present invention, or a pharmaceutically acceptable salt thereof.

In some embodiments, the present invention provides a method of treating malignant rhabdoid tumors (MRT) in a patient in need thereof, comprising administering a compound of the present invention, or a pharmaceutically acceptable salt thereof.

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds according to the invention are useful in the treatment of heteroimmune diseases. Examples of such heteroimmune diseases include, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example anti-inflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterized by asthma attack, e.g. between the hours of about 4 to 6 am, i.e. at a time normally substantially distant form any previously administered symptomatic asthma therapy.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods of this invention is a disease of the skin. In some embodiments, the inflammatory disease of the skin is selected from contact dermatitis, atopic dermatitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, and other inflammatory or allergic conditions of the skin.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In some embodiments the inflammatory disease which can be treated according to the methods of this invention is a TH17 mediated disease. In some embodiments the TH17 mediated disease is selected from Systemic lupus erythematosus, Multiple sclerosis, and inflammatory bowel disease (including Crohn's disease or ulcerative colitis).

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

Cardiovascular diseases which can be treated according to the methods of this invention include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the methods of this invention include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In some embodiments the invention provides a method of treating, preventing or lessening the severity of Alzheimer's disease comprising administering to a patient in need thereof a provided compound or a pharmaceutically acceptable salt or composition thereof.

In some embodiments the invention provides a method of treating a disease or condition commonly occurring in connection with transplantation. In some embodiments, the disease or condition commonly occurring in connection with transplantation is selected from organ transplantation, organ transplant rejection, and graft versus host disease.

In some embodiments the invention provides a method of treating a metabolic disease. In some embodiments the metabolic disease is selected from Type 1 diabetes, Type 2 diabetes, metabolic syndrome, and obesity.

In some embodiments the invention provides a method of treating a viral disease. In some embodiments, the viral infection is HIV infection.

Furthermore, the invention provides the use of a compound according to the definitions herein, or a pharmaceutically acceptable salt, or a hydrate or solvate thereof for the preparation of a medicament for the treatment of a proliferative disease, an inflammatory disease, an obstructive respiratory disease, a cardiovascular disease, a metabolic disease, a neurological disease, a neurodegenerative disease, a viral disease, or a disorder commonly occurring in connection with transplantation.

Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketoconazole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenstrom's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine ophthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting SWI/SNF chromatin-remodeling complex activity or degrading a SWI/SNF chromatin-remodeling complex in a biological sample comprising the step of contacting said biological sample with a compound of this invention, or a composition comprising said compound.

According to another embodiment, the invention relates to a method of inhibiting or degrading SMARCA2, SMARCA4, or PB1, or a mutant thereof, activity in a biological sample comprising the step of contacting said biological sample with a compound of this invention, or a composition comprising said compound.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition and/or degradation of a SMARCA or PB1 protein, or a protein selected from SMARCA2, SMARCA4, or PB1, or a mutant thereof, activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of degrading a protein kinase and/or inhibiting protein kinase activity in a patient comprising the step of administering to said patient a compound of the present invention, or a composition comprising said compound.

According to another embodiment, the invention relates to a method of degrading and/or inhibiting one or more SMARCA2, SMARCA4, or PB1, or a mutant thereof, activity in a patient comprising the step of administering to said patient a compound of the present invention, or a composition comprising said compound. In other embodiments, the present invention provides a method for treating a disorder mediated by one or more SMARCA2, SMARCA4, or PB1, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™) The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporne derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ- tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zamestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™), ); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-O-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]- amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden),V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID(TM) CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MED14736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS.F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-lh68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8 ⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those described in Jerry L. Adams et al., “Big opportunities for small molecules in immuno-oncology,” Cancer Therapy 2015, Vol. 14, pages 603-622, the content of which is incorporated herein by reference in its entirety. In some embodiment, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (a) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron -4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
″BuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAI: tetrabutylammonium iodide
TEA: triethylamine
Tf: trifluoromethanesulfonate
TfAA, TFMSA or Tf₂O: trifluoromethanesulfonic anhydride
TFA: trifluoracetic acid
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
wt: weight
Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

TABLE 2
Analytical instruments

| | |
|---|---|
| LCMS | Shimadzu UFLC MS: LCMS-2020 |
| | Agilent Technologies 1200 series MS: Agilent |
| | Technologies 6110 |
| | Agilent Technologies 1200 series MS: LC/MSD VL |
| NMR | BRUKER AVANCE III/400; Frequency (MHz) |
| | 400.13; Nucleus: 1H; Number of Transients: 8 |
| Prep-HPLC | Gilson GX-281 systems: instruments GX-A, GX-B, |
| | GX-C, GX-D, GX-E, GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH+] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH+] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19)mm, 5. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Intermediates:

### Ethyl 3-methyl-2-(3-((4-oxocyclohexyl)methoxy)isoxazol-5-yl)butanoate (Intermediate A)

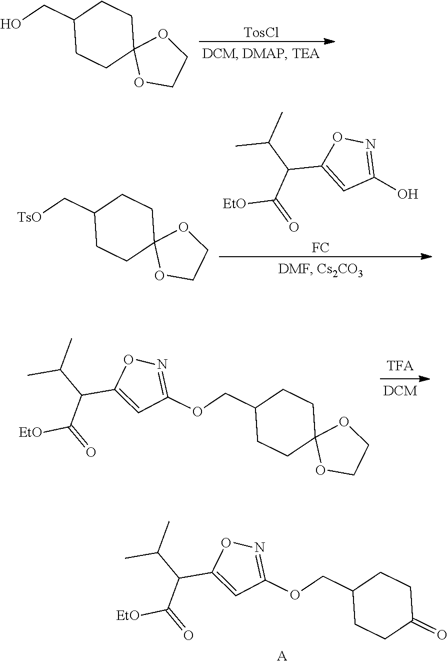

Step 1-1,4-dioxaspiro[4.5]decan-8-ylmethyl 4-methylbenzenesulfonate. To a solution of 1,4-dioxaspiro[4.5]decan-8-ylmethanol (4.30 g, 24.9 mmol) in DCM (80 mL) was added TosCl (5.71 g, 29.9 mmol), TEA (5.05 g, 49.9 mmol) and DMAP (183 mg, 1.50 mmol). The mixture was stirred at 0° C. for 2 hours. On completion, the reaction mixture was diluted with water 150 mL and extracted with DCM (100 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=50/1 to 10/1) to give the title compound (4.2 g, 52% yield) as yellow solid. ¹H NMR (400 MHz, CHLOROFORM-d) δ 7.71 (d, J=8.0 Hz, 2H) 7.27 (d, J=8.0 Hz, 2H) 3.79-3.89 (m, 4H) 3.77 (d, J=8.0 Hz, 2H) 2.38 (s, 3H) 1.59-1.70 (m, 5H) 1.42 (m, 2H) 1.15 (m, 2H).

Step 2-Ethyl 2-(3-(1,4-dioxaspiro[4.5]decan-8-ylmethoxy)isoxazol-5-yl)-3-methylbutanoate. To a solution of 1,4-dioxaspiro[4.5]decan-8-ylmethyl 4-methylbenzenesulfonate (4.20 g, 12.8 mmol) in DMF (50 mL) was added Cs₂CO₃ (8.38 g, 25.7 mmol) and ethyl 2-(3-hydroxyisoxazol-5-yl)-3-methyl-butanoate (3.29 g, 15.4 mmol, Intermediate FC). The mixture was stirred at 70° C. for 3 hours. On completion, the reaction mixture was diluted with H₂O (10 mL) and extracted with EA (70 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=20/1 to 10/1) to give the title compound (2.9 g, 61% yield) as a yellow solid. LC-MS (ESI⁺) m/z 368.6 (M+H)⁺.

Step 3-Ethyl 3-methyl-2-(3-((4-oxocyclohexyl)methoxy)isoxazol-5-yl)butanoate. To a solution of ethyl 2-[3-(1,4-dioxaspiro[4.5]decan-8-ylmethoxy)isoxazol-5-yl]-3-methyl-butanoate (2.90 g, 7.89 mmol) in DCM (40 mL) was added TFA (10 mL). The mixture was stirred at 25° C. for 12 hours. On completion, the reaction mixture was concentrated under reduced pressure to give a residue. The residue was purified by prep-HPLC (0.1% TFA condition) to give the title compound (1.63 g, 62% yield, TFA) as a white solid. LC-MS (ESI⁺) m/z 324.1 (M+H)⁺.

### (R)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (Intermediate B) and ((S)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (Intermediate O)

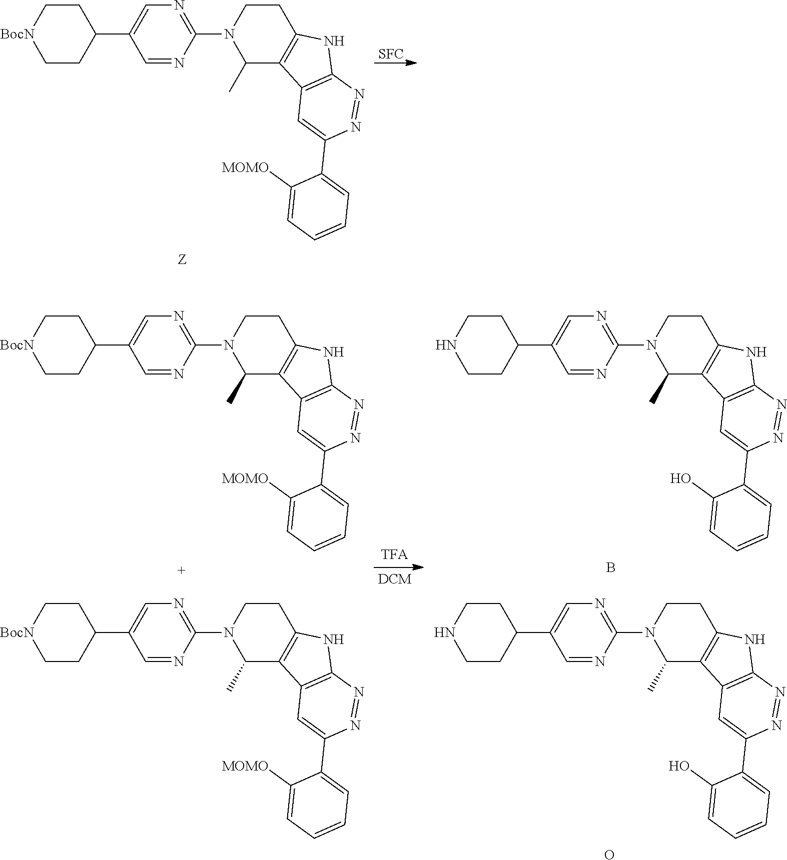

Step 1-(R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidine-1-carboxylate acid and (S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidine-1-carboxylate. Tert-butyl 4-[2-[12-[2-(methoxymethoxy)phenyl]-3-methyl-4,8,10,11-tetrazatricyclo[7.4.0.02,7]trideca-1(9),2(7),10,12-tetraen-4-yl]pyrimidin-5-yl]piperidine-1-carboxylate (560 mg, 956 umol, Intermediate Z) was separated by SFC (column: DAICEL CHIRALPAK AS (250 mm*30 mm, 10 um); mobile phase: [0.1% NH₃H₂O, MeOH]; B %: 60%-60%, 6.5; 80 min) to give (R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidine-1-carboxylate acid (260 mg, 444 umol, 46% yield) as a white solid. LC/MS (ESI, m/z): [M+1]+=586.6 and (S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidine-1-carboxylate (260 mg, 444 umol, 46% yield) as a white solid. LC/MS (ESI, m/z): [M+1]+=586.6.

Step 2-(R)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol and (S)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol. To a solution of (R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidine-1-carboxylate (80.0 mg, 137 umol) in DCM (2 mL) was added HCl/dioxane (4 M, 0.5 mL). The mixture was stirred at 25° C. for 0.5 hour. On completion, the reaction mixture was concentrated under reduced pressure to give a residue. The residue was purified by prep-HPLC (column: Welch Xtimate C18 150*25 mm*5 um; mobile phase: [water (0.05% HCl)-ACN];B %: 2%-32%,10 min) to give the title compound (23 mg, 35.1% yield, HCl). ¹H NMR (400 MHz, DMSO-d₆) δ=13.77 (s, 1H), 9.33-8.92 (m, 2H), 8.73 (s, 1H), 8.35 (s, 2H), 7.72-7.60 (m, 1H), 7.51-7.39 (m, 1H), 7.19 (d, J=8.0 Hz, 1H), 7.06 (dt, J=0.8, 7.6 Hz, 1H), 6.12 (q, J=6.4 Hz, 1H), 5.15-5.01 (m, 1H), 3.50-3.42 (m, 1H), 3.33 (d, J=12.4 Hz, 2H), 3.14-3.07 (m, 2H), 2.95 (q, J=11.6 Hz, 2H), 2.81-2.72 (m, 1H), 1.95-1.80 (m, 4H), 1.54 (d, J=6.4 Hz, 3H). LC/MS (ESI, m/z): [M+1]⁺=442.4.

Step 3-(S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidine-1-carboxylate was deprotected according to the same method to give the title compound (29.2 mg, 44% yield, HCl salt) as a yellow solid. ¹H NMR (400 MHz, DMSO-d₆) δ=13.78-13.52 (m, 1H), 9.09-8.91 (m, 1H), 8.90-8.77 (m, 1H), 8.73 (s, 1H), 8.34 (s, 2H), 7.70 (d, J=7.6 Hz, 1H), 7.52-7.42 (m, 1H), 7.15 (d, J=8.0 Hz, 1H), 7.06 (dt, J=0.8, 7.6 Hz, 1H), 6.11 (q, J=6.4 Hz, 1H), 5.15-5.03 (m, 1H), 3.52-3.41 (m, 1H), 3.34 (d, J=12.0 Hz, 2H), 3.14-3.05 (m, 2H), 3.02-2.90 (m, 2H), 2.76 (tt, J=3.6, 12.0 Hz, 1H), 1.96-1.75 (m, 4H), 1.54 (d, J=6.4 Hz, 3H). LC/MS (ESI, m/z): [M+1]=442.3. Absolute stereochemistry was assigned arbitrarily.

### 2-(3-((4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)-methoxy)isoxazol-5-yl)-3-methylbutanoic acid (Intermediate C)

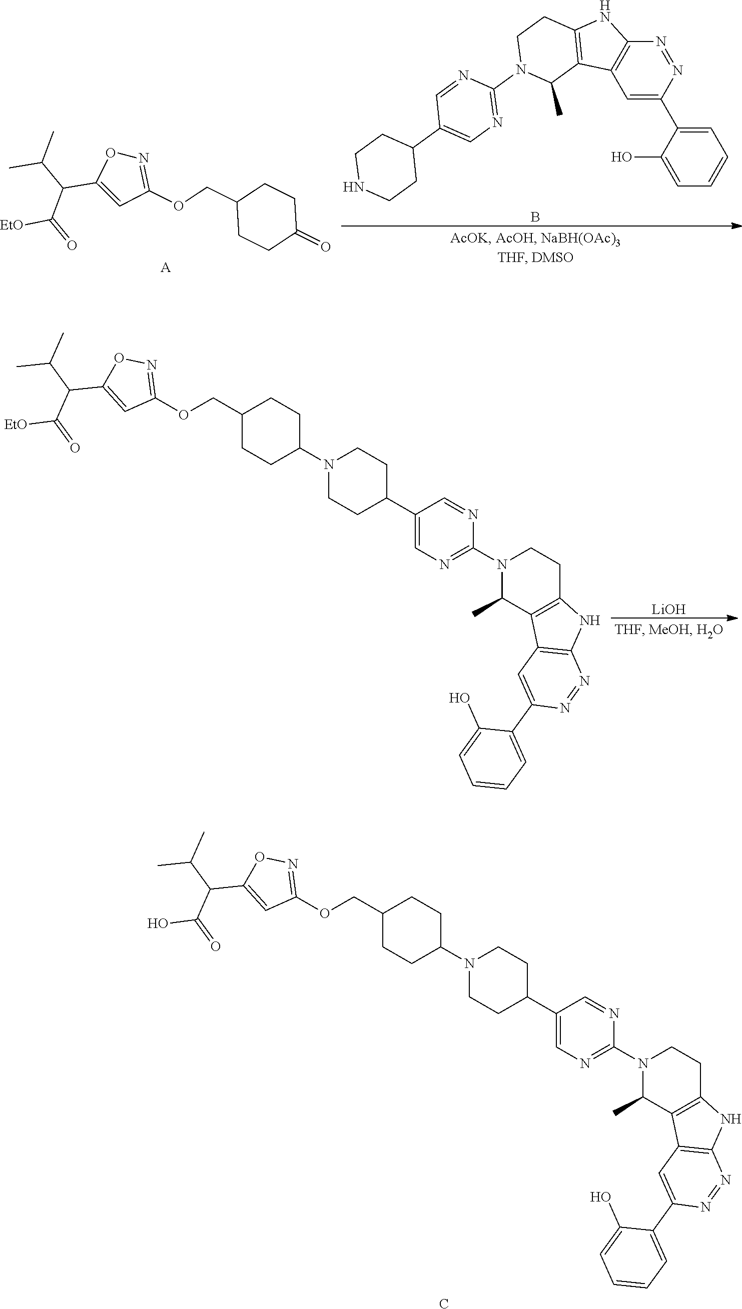

Step 1-Ethyl 2-(3-((4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)-methoxy)isoxazol-5-yl)-3-methylbutanoate. To a solution of 2-[(3R)-3-methyl-4-[5-(4-piperidyl)pyrimidin-2-yl]-4,8,10,11-tetrazatricyclo[7.4.0.0^{2,7}]trideca-1(9),2(7),10,12-tetraen-12-yl]phenol (295 mg, 618 umol, HCl, Intermediate B) in DMSO (0.5 mL) and THF (2 mL) was added AcOK (91.0 mg, 927 umol) and the mixture was stirred for 30 minutes. Then ethyl 3-methyl-2-[3-[(4-oxocyclohexyl)methoxy]isoxazol-5-yl]butanoate (200 mg, 618 umol, Intermediate A) and AcOH (55.7 mg, 927 umol) was added and the mixture was stirred 1 hour. Next, NaBH(OAc)₃ (393 mg, 1.86 mmol) was added at 0° C. and the mixture was stirred at 50° C. for 1.5 hours. On completion, the reaction mixture was filtered and concentrated under reduced pressure to give a residue. The residue was purified by prep-HPLC (neutral condition column: Waters xbridge 150*25 mm 10 um; mobile phase: [water(10 mM NH₄HCO₃)-ACN]; B %: 67%-87%, 11 min) to give the title compound (80 mg, 30% yield) as white solid. LC-MS (ESI⁺) m/z 749.4 (M+H)⁺.

Step 2-2-(3-((4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)-methoxy)isoxazol-5-yl)-3-methylbutanoic acid. To a solution of ethyl 2-[3-[[4-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]cyclohexyl]methoxy]isoxazol-5-yl]-3-methyl-butanoate (80 mg, 106 umol) in THF (0.5 mL), MeOH (0.5 mL) and H₂O (0.5 mL) was added LiOH·H₂O (6.72 mg, 160 umol). The reaction mixture was stirred at 25° C. for 2 hours. On completion, the reaction mixture was adjusted the pH to 7 and concentrated under reduced pressure to give the title compound (80 mg) as a yellow solid. LC-MS (ESI⁺) m/z 721.6 (M+H)⁺.

### (2S,4R)-4-hydroxy-N-[[4-(4-methylthiazol-5-yl)phenyl]methyl]pyrrolidine-2-carboxamide (Intermediate D) (CAS #1448189-90-9)

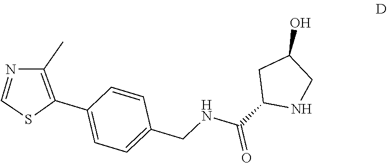 [SMILES: Cc1ncsc1-c1ccc(CNC(=O)[C@@H]2C[C@@H](O)CN2)cc1]

### Tert-butyl 4-[3-(2-hydroxyphenyl)-9H-pyridazino[3,4-b]indol-6-yl]-3,6-dihydro-2H-pyridine-1-carboxylate (Intermediate E)

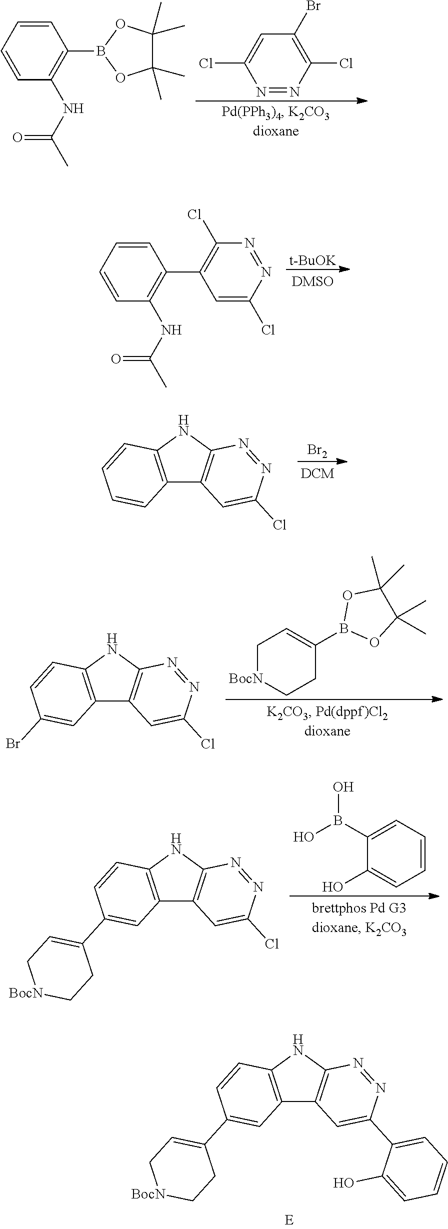

Step 1-N-(2-(3,6-dichloropyridazin-4-yl)phenyl)acetamide. To a solution of N-[2-(4,4,5,5-tetramethyl-1,3,2-dioxaborolan-2-yl)phenyl]acetamide (1.8 g, 6.89 mmol, CAS #380430-61-5) in dioxane (5 mL) was added 4-bromo-3,6-dichloro-pyridazine (2.04 g, 8.96 mmol, CAS #10344-42-0), Pd(PPh₃)₄(796 mg, 689 umol) and K₂CO₃ (2 M, 10.3 mL), then the mixture was stirred at 85° C. for 12 hours. On completion, the residue was diluted with ethyl acetate (150 mL) and extracted with water (150 mL). The combined organic layers were washed with brine (100 mL), dried over Na₂SO₄, filtered and concentrated under reduced pressure to give a residue. The residue was purified by column chromatography (SiO₂, Petroleum ether/Ethyl acetate=2/1) to give title compound (1.2 g, 56% yield) as a pink solid LC-MS (ESI⁺) m/z 282.0 (M+H)⁺.

Step 2-3-Chloro-9H-pyridazino[3,4-b]indole. To a solution of N-[2-(3,6-dichloropyridazin-4-yl)phenyl]acetamide (1.2, 4.25 mmol) in DMSO (10 mL) was added t-BuOK (1.19 g, 10.6 mmol), then the mixture was stirred at 45° C. for 12 hours. On completion, the mixture was diluted with NH₄Cl. The residue was diluted with ethyl acetate (200 mL) and extracted with water (150 mL). The combined organic layers were washed with brine (150 mL), dried over Na₂SO₄, filtered and concentrated under reduced pressure to give the title compound (750 mg) as a purple solid. LC-MS (ESI⁺) m/z 204.0 (M+H)⁺.

Step 3-6-Bromo-3-chloro-9H-pyridazino[3,4-b]indole. To a solution of 3-chloro-9H-pyridazino[3,4-b]indole (750 mg, 3.68 mmol) in DCM (1 mL) was added Br₂ (647 mg, 4.05 mmol), then the mixture was stirred at 0-25° C. for 2 hours. On completion, the reaction mixture was filtered and concentrated under reduced pressure to give the title compound (650 mg) as a yellow solid. LC-MS (ESI*) m/z 283.7 (M+H)⁺.

Step 4-Tert-butyl 4-(3-chloro-9H-pyridazino[3,4-b]indol-6-yl)-5,6-dihydropyridine-1(2H)-carboxylate. To a solution of 6-bromo-3-chloro-9H-pyridazino[3,4-b]indole (650 mg, 2.30 mmol) in dioxane (4 mL) and H₂O (1 mL) was added tert-butyl 4-(4,4,5,5-tetramethyl-1,3,2-dioxaborolan-2-yl)-3,6-dihydro-2H-pyridine-1-carboxylate (853 mg, 2.76 mmol) and Pd(dppf)Cl₂ (168 mg, 230 umol), K₂CO₃ (953 mg, 6.90 mmol), then the mixture was stirred at 85° C. for 12 hours. On completion, the residue was diluted with DCM (40 mL) and extracted with water (50 mL). The combined organic layers were washed with brine (40 mL), dried over Na₂SO₄, filtered and concentrated under reduced pressure to give a residue. The residue was purified by column chromatography (SiO₂, Petroleum ether/Ethyl acetate=3/1) to give title compound (280 mg, 25% yield) as a yellow solid. LC-MS (ESI⁺) m/z 385.1 (M+H)⁺.

Step 5-Tert-butyl 4-(3-(2-hydroxyphenyl)-9H-pyridazino[3,4-b]indol-6-yl)-5,6-dihydropyridine-1(2H)-carboxylate. To a solution of tert-butyl 4-(3-chloro-9H-pyridazino[3,4-b]indol-6-yl)-3,6-dihydro-2H-pyridine-1-carboxylate (150 mg, 389 umol) in dioxane (4 mL) and H₂O (1 mL) was added (2-hydroxyphenyl)boronic acid (80.6 mg, 584 umol) and K₂CO₃ (161 mg, 1.17 mmol), BrettPhos Pd G3 (35.3 mg, 38.9 umol), then the mixture was stirred at 100° C. for 12 hours. On completion, the residue was diluted with water (50 mL) and extracted with DCM (60 mL). The combined organic layers were washed with brine (40 mL), dried over Na₂SO₄, filtered and concentrated under reduced pressure to give a residue. The residue was purified by column chromatography (SiO₂, Petroleum ether/Ethyl acetate=3/1) to give (60 mg, 28% yield) as a yellow solid. LC-MS (ESI⁺) m/z 443.1 (M+H)⁺.

### 2-(6-([1,4′-bipiperidin]-4-yl)-9H-pyridazino[3,4-b]indol-3-yl)phenol (Intermediate F)

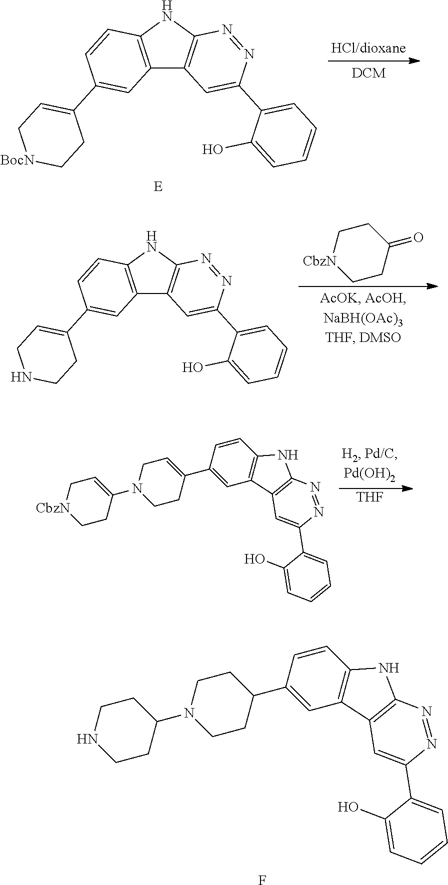

Step 1-2-(6-(1,2,3,6-Tetrahydropyridin-4-yl)-9H-pyridazino[3,4-b]indol-3-yl)phenol. To a solution of tert-butyl 4-[3-(2-hydroxyphenyl)-9H-pyridazino[3,4-b]indol-6-yl]-3,6-dihydro-2H-pyridine-1-carboxylate (1.00 g, 2.26 mmol, Intermediate E) in DCM (10 mL) was added HCl/dioxane (4 M, 10 mL). The mixture was stirred at 25° C. for 2 hours. On completion, the reaction mixture was filtered with DCM and concentrated under reduced pressure to give the title compound (1.00 g, HCl) as yellow solid. LC-MS (ESI⁺) m/z 343.3 (M)⁺.

Step 2-Benzyl 4-(3-(2-hydroxyphenyl)-9H-pyridazino[3,4-b]indol-6-yl)-3,5′,6,6′-tetrahydro-2H-[1,4′-bipyridine]-1′(2′H)-carboxylate. To a solution of 2-[6-(1,2,3,6-tetrahydropyridin-4-yl)-9H-pyridazino[3,4-b]indol-3-yl]phenol (700 mg, 1.85 mmol, HCl) in THF (15 mL) and DMSO (2 mL) was added AcOK (544 mg, 5.54 mmol) and the mixture was stirred at 50° C. for 30 minutes. Then benzyl 4-oxopiperidine-1-carboxylate (646 mg, 2.77 mmol) and AcOH (333 mg, 5.54 mmol) was added and the mixture was stirred for 2 hours. Next, NaBH(OAc)₃ (979 mg, 4.62 mmol) was added at 0° C. Then the mixture was stirred at 50° C. for 3 hours. On completion, the reaction mixture was filtered and concentrated under reduced pressure to give a residue. The residue was purified by prep-HPLC (0.1% NH₃·H₂O condition) to give the title compound (400 mg, 38% yield) as a yellow solid. LC-MS (ESI⁺) m/z 560.3 (M+H)⁺.

Step 3-2-(6-([1,4′-Bipiperidin]-4-yl)-9H-pyridazino[3,4-b]indol-3-yl)phenol. To a solution of benzyl 4-[4-[3-(2-hydroxyphenyl)-9H-pyridazino[3,4-b]indol-6-yl]-3,6-dihydro-2H-pyridin-1-yl]piperidine-1-carboxylate (400 mg, 714 umol) in THF (50 mL) was added Pd/C (200 mg, 714 umol, 10 wt %) and Pd(OH)₂ (200 mg, 1.43 mmol) under N₂ atmosphere. The mixture was stirred at 25° C. for 48 hours under H₂ (15 psi) atmosphere. On completion, the reaction mixture was filtered with THF and DCM and concentrated under reduced pressure to give the title compound (300 mg) as yellow solid. LC-MS (ESI⁺) m/z 428.1 (M+H)⁺.

### 6-(4-(3-(2-hydroxyphenyl)-9H-pyridazino[3,4-b]indol-6-yl)-[1,4′-bipiperidin]-1′-yl)spiro[3.3]heptane-2-carboxylic acid (Intermediate G)

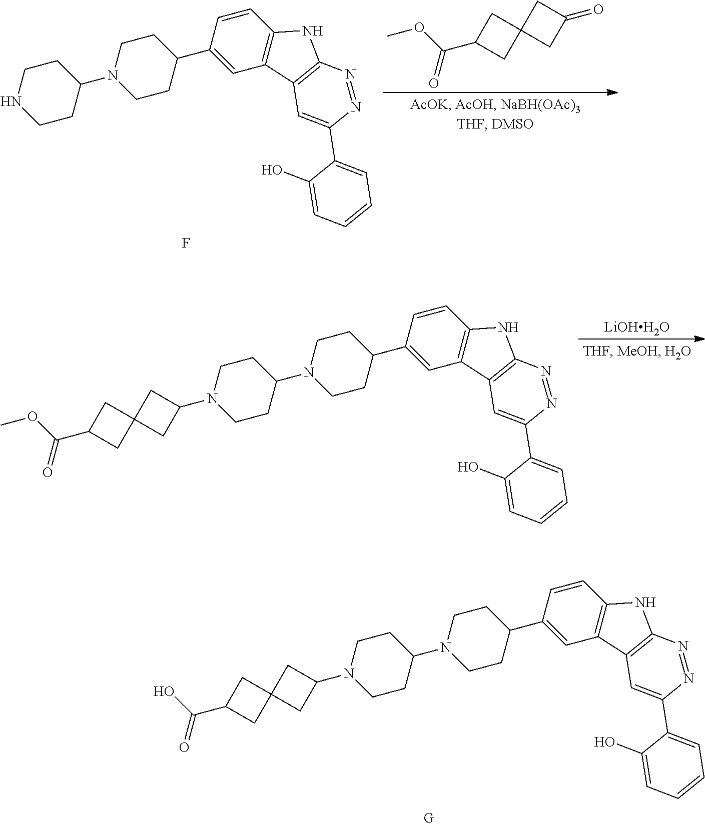

Step 1-Methyl 6-(4-(3-(2-hydroxyphenyl)-9H-pyridazino[3,4-b]indol-6-yl)-[1,4′-bipiperidin]-1′-yl)spiro[3.3]heptane-2-carboxylate. To a solution of2-[6-[1-(4-piperidyl)-4-piperidyl]-9H-pyridazino[3,4-b]indol-3-yl]phenol (300 mg, 701 umol), methyl 2-oxospiro[3.3]heptane-6-carboxylate (129 mg, 771 umol, CAS #1138480-98-4) in THF (3 mL) and DMSO (0.3 mL) was added AcOH (105 mg, 1.75 mmol) and the mixture was stirred at 50° C. for 2 hours. Next, NaBH(OAc)₃ (446 mg, 2.11 mmol) was added at 0° C., and the mixture was then stirred at 50° C. for 3 hours. On completion, the reaction mixture was filtered and concentrated under reduced pressure to give a residue. The residue was purified by prep-HPLC (0.1% FA condition) to give the title compound (150 mg, 36% yield) as a yellow solid. LC-MS (ESI⁺) m/z 580.2.

Step 2-6-(4-(3-(2-Hydroxyphenyl)-9H-pyridazino[3,4-b]indol-6-yl)-[1,4′-bipiperidin]-1′-yl)spiro[3.3]heptane-2-carboxylic acid. To a solution of methyl 2-[4-[4-[3-(2-hydroxyphenyl)-9H-pyridazino[3,4-b]indol-6-yl]-1-piperidyl]-1-piperidyl]spiro[3.3]heptane-6-carboxylate (150 mg, 258 umol) in THF (2 mL), H₂O (2 mL) and MeOH (2 mL) was added LiOH—H₂O (27.1 mg, 646 umol). The mixture was stirred at 25° C. for 2 hours. On completion, the reaction mixture was filtered and concentrated under reduced pressure to give the title compound (150 mg) as a yellow solid. LC-MS (ESI⁺) m/z 566.3.

### (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (Intermediate H) (CAS #1448189-80-7)

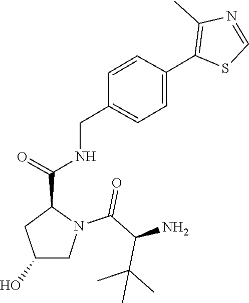 [SMILES: Cc1ncsc1-c1ccc(CNC(=O)[C@@H]2C[C@@H](O)CN2C(=O)[C@@H](N)C(C)(C)C)cc1]

### Ethyl 3-methyl-2-(3-(4-(2-oxoethyl)piperidin-1-yl)isoxazol-5-yl)butanoate (Intermediate I)

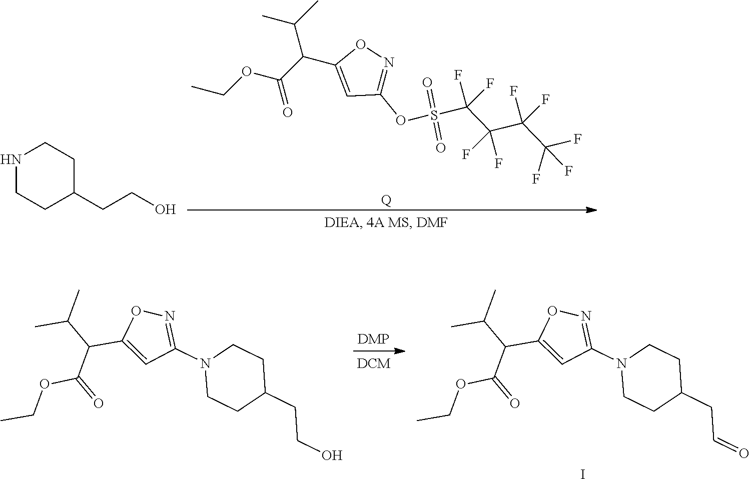

Step 1-Ethyl 2-(3-(4-(2-hydroxyethyl)piperidin-1-yl)isoxazol-5-yl)-3-methylbutanoate. To a solution of 2-(4-piperidyl)ethanol (782 mg, 6.06 mmol, CAS #622-26-4) and ethyl 3-methyl-2-[3-(1,1,2,2,3,3,4,4,4-nonafluorobutylsulfonyloxy)isoxazol-5-yl]butanoate (3.00 g, 6.06 mmol, Intermediate Q) in DMF (20 mL) was added DIEA (2.35 g, 18.2 mmol) and 4 Å molecular sieves (3.00 g). The mixture was stirred at 130° C. for 2 hours. On completion, the reaction was filtered and the filtration was extracted with ethyl acetate (30 mL×3) and H₂O (10 mL). The combined organic layers were washed with brine (30 mL×3), dried over anhydrous sodium sulfate, filtered and concentrated under reduced pressure to give a residue. The residue was purified by column chromatography (SiO₂, Petroleum ether/Ethyl acetate=10/1 to 2/1) to give the title compound (520 mg, 26% yield) as a yellow oil. LC-MS (ESI⁺) m/z 325.6 (M+H)⁺.

Step 2-Ethyl 3-methyl-2-(3-(4-(2-oxoethyl)piperidin-1-yl)isoxazol-5-yl)butanoate. To a solution of ethyl 2-[3-[4-(2-hydroxyethyl)-1-piperidyl]isoxazol-5-yl]-3-methyl-butanoate (200 mg, 616 umol) in DCM (10 mL) was added DMP (314 mg, 740 umol) at 0° C. The mixture was stirred at 25° C. for 3 hours. The reaction mixture was quenched with saturated NaS₂O₃ aqueous solution (20 mL) and saturated NaHCO₃ aqueous solution (20 mL), and then extracted with DCM (20 mL×3). The combined organic layers were washed by NaHCO₃ aqueous solution (20 mL×3), dried over with anhydrous sodium sulfate, filtered and concentrated under reduced pressure to give the title compound (230 mg) as a yellow oil. LC-MS (ESI⁺) m/z 323.2 (M+H)⁺.

### 2-(3-(4-(2-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)ethyl)piperidin-1-yl)isoxazol-5-yl)-3-methylbutanoic acid (Intermediate J)

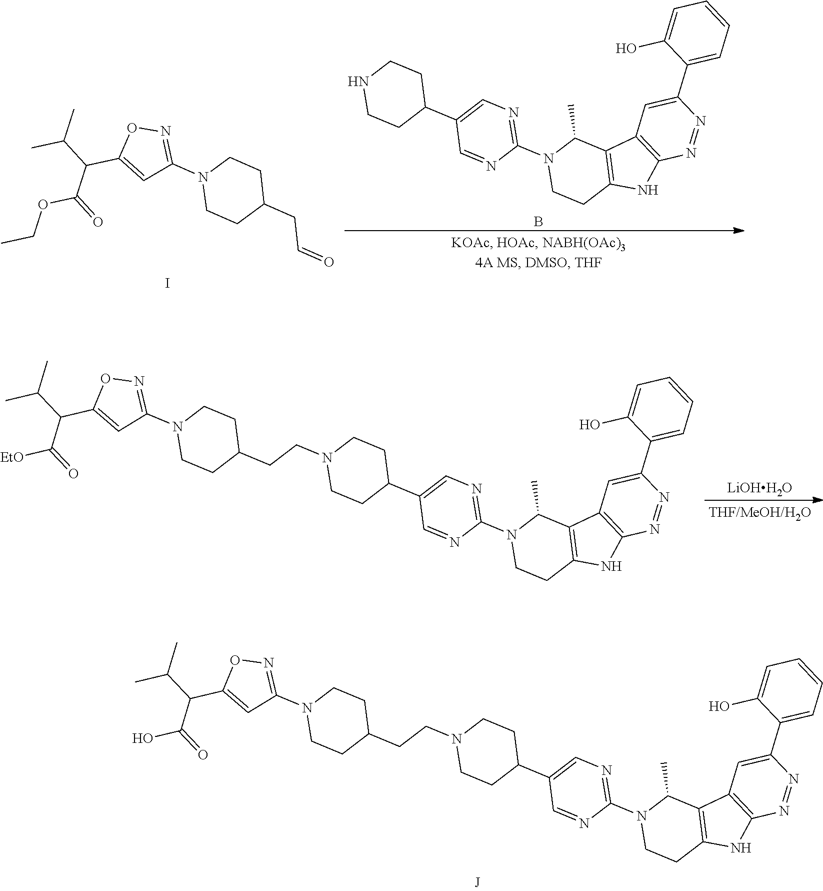

Step 1-Ethyl 2-(3-(4-(2-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)ethyl)piperidin-1-yl)isoxazol-5-yl)-3-methylbutanoate. To a solution of ethyl 3-methyl-2-[3-[4-(2-oxoethyl)-1-piperidyl]isoxazol-5-yl]butanoate (230 mg, 713 umol, Intermediate I) in THF (5 mL) and DMSO (1 mL) was added AcOK (210 mg, 2.14 mmol), and the reaction was stirred for 30 minutes at 0° C. Then 2-[(3R)-3-methyl-4-[5-(4-piperidyl)pyrimidin-2-yl]-4,8,10,11-tetrazatricyclo[7.4.0.02,7]trideca-1(9),2(7),10,12-tetraen-12-yl]phenol (252 mg, 571 umol, Intermediate B), AcOH (128 mg, 2.14 mmol) and 4 Å molecular sieves (300 mg) were added, and the reaction was stirred at 0° C. for 30 minutes. Next, NaBH(OAc)₃ (378 mg, 1.80 mmol) was added, and the resulting solution was stirred at 0° C. for 2 hours. On completion, the reaction mixture was quenched with MeOH (5 mL), and then extracted with EA (10 mL×3). The combined organic layers were washed with brine (10 mL×3), dried over with Na₂SO₄, filtered and concentrated under reduced pressure to give a residue. The residue was purified by column chromatography (SiO₂, DCM:MeOH=10/1 to 2/1) to give the title compound (210 mg, 38% yield) as a yellow oil. LC-MS (ESI⁺) m/z 748.7 (M+H)⁺.

Step 2-2-(3-(4-(2-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)ethyl)piperidin-1-yl)isoxazol-5-yl)-3-methylbutanoic acid. To a solution of ethyl 2-[3-[4-[2-[4-[2-[(3R)-12-(2-hydroxyphenyl)-3-methyl-4,8,10,11-tetrazatricyclo[7.4.0.02,7]trideca-1(9),2(7),10,12-tetraen-4-yl]pyrimidin-5-yl]-1-piperidyl]ethyl]-1-piperidyl]isoxazol-5-yl]-3-methyl-butanoate (200 mg, 267 umol) in a mixture solution of H₂O (3 mL), MeOH (3 mL) and THF (3 mL) was added LiOH·H₂O (56.1 mg, 1.34 mmol). After the reaction was stirred at 25° C. for 2 hours, the reaction mixture was concentrated under reduced pressure to remove most of the organic solvents. Then the mixture was dried by lyophilization to give the title compound (230 mg) as a yellow solid. LC-MS (ESI⁺) m/z 720.6 (M+H)⁺.

### (2S,4R)-4-hydroxy-1-[2-(3-hydroxyisoxazol-5-yl)-3-methyl-butanoyl]-N-[[4-(4-methylthiazol-5-yl)phenyl]methyl]pyrrolidine-2-carboxamide (Intermediate K)

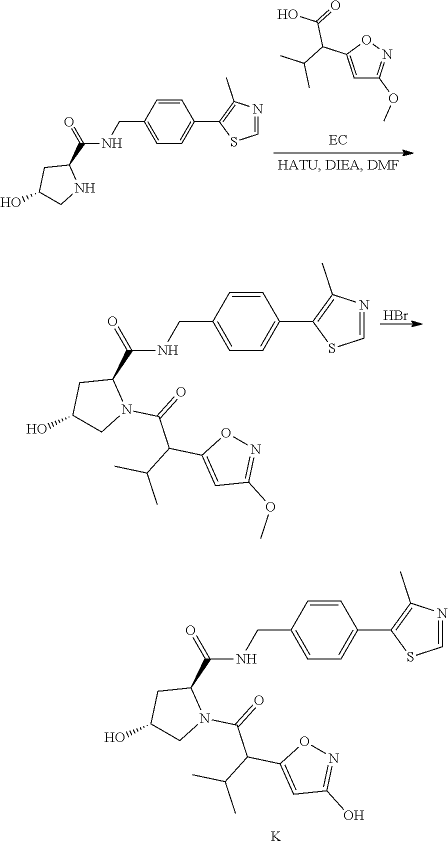

Step 1-(2S,4R)-4-hydroxy-1-(2-(3-methoxyisoxazol-5-yl)-3-methylbutanoyl)-N-(4-(4-methylthiazol-5-yl)benzyl)pyrrolidine-2-carboxamide. To a solution of 2-(3-methoxyisoxazol-5-yl)-3-methyl-butanoic acid (1 g, 5.02 mmol, Intermediate EC) in DMF (10 mL) was added HATU (2.10 g, 5.52 mmol) and DIEA (3.89 g, 30.12 mmol, 5.25 mL). Then (2S,4R)-4-hydroxy-N-[[4-(4-methylthiazol-5-yl)phenyl]methyl]pyrrolidine-2-carboxamide (1.78 g, 5.02 mmol, CAS #: 1448189-90-9) was added and the mixture was stirred at 25° C. for 12 hours. On completion, the reaction mixture was quenched with water (50 mL) and extracted with ethyl acetate (3×50 mL). The extracts were washed by brine (3×30 mL) and dried over anhydrous sodium sulfate, filtered and concentrated in vacuo to give a residue. The residue was purified by silica gel chromatography (petroleum ether:ethyl acetate=5:1 to 1:1) to give the title compound (6 g, 96% yield) as a yellow solid. LC/MS (ESI, m/z): [M+1]⁺=499.2.

Step 2-(2S,4R)-4-hydroxy-1-(2-(3-hydroxyisoxazol-5-yl)-3-methylbutanoyl)-N-(4-(4-methylthiazol-5-yl)benzyl)pyrrolidine-2-carboxamide. To a mixture of (2S,4R)-4-hydroxy-1-[2-(3-methoxyisoxazol-5-yl)-3-methyl-butanoyl]-N-[[4-(4-methylthiazol-5-yl)phenyl]methyl]pyrrolidine-2-carboxamide (1 g, 2.01 mmol) was added HBr (7 mL, 40% solution) in one portion at 25° C. Then the mixture was stirred at 60° C. for 12 hours. On completion, the reaction mixture was concentrated to give the title compound (950 mg) as an orange solid. LC/MS (ESI, m/z): [M+1]⁺=485.1.

### (2S,4R)-1-(2-(3-(4-bromobutoxy)isoxazol-5-yl)-3-methylbutanoyl)-4-hydroxy-N-(4-(4-methylthiazol-5-yl)benzyl)pyrrolidine-2-carboxamide (Intermediate L)

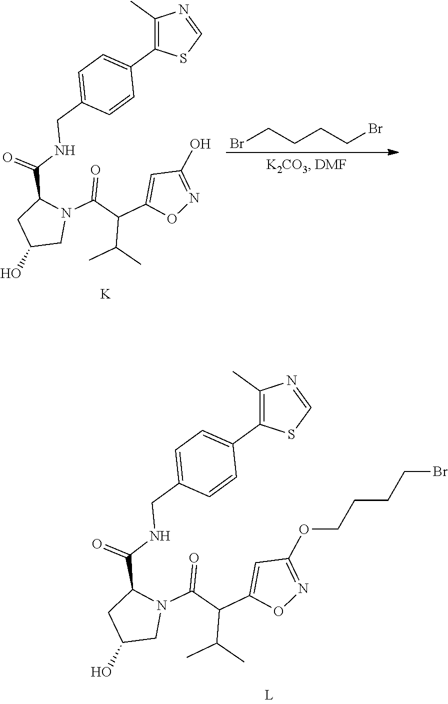

To a solution of (2S,4R)-4-hydroxy-1-[2-(3-hydroxyisoxazol-5-yl)-3-methyl-butanoyl]-N-[[4-(4-methylthiazol-5-yl)phenyl]methyl]pyrrolidine-2-carboxamide (200 mg, 412 umol, Intermediate K) in DMF (2 mL) was added K₂CO₃ (171 mg, 1.24 mmol) and 1,4-dibromobutane (267 mg, 1.24 mmol, CAS #110-52-1). The mixture was stirred at 80° C. for 2 hours. On completion, the reaction mixture was filtered to give a filtrate. The filtrate was purification by prep-HPLC(FA condition) to give the title compound (150 mg, 58% yield) as a white solid. LC-MS (ESI⁺) m/z 619.3 (M+H)⁺.

### 2-[(10S)-12-[5-(4-piperidyl)pyrimidin-2-yl]-1,5,6,8,12-pentazatricyclo[8.4.0.0^{2,7}]tetradeca-2(7),3,5-trien-4-yl]phenol (Intermediate M)

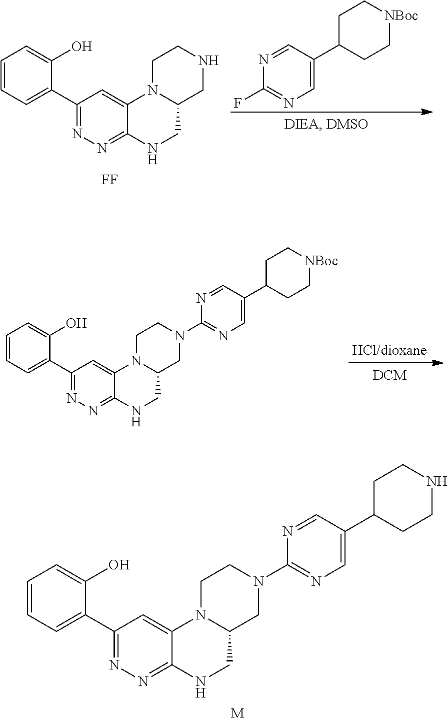

Step 1-Tert-butyl 2-chloro-6-oxo-6a,7,9,10-tetrahydro-5H-pyrazino[1′,2′:4,5]pyrazino[2,3-c]pyridazine-8(6H)-carboxylate. To a solution of 2-(1,5,6,8,12-pentazatricyclo[8.4.0.02,7]tetradeca-2,4,6-trien-4-yl)phenol (4 g, 12.5 mmol, Intermediate FF) in DMSO (50 mL) was added tert-butyl 4-(2-chloropyrimidin-5-yl)piperidine-1-carboxylate (5.59 g, 18.7 mmol, Intermediate FG) and DIEA (8.08 g, 62.5 mmol), then the reaction was stirred at 60° C. for 12 hours. On completion, aqueous NaCl was added into the solution and the solution was filtered. Then the filtrate was collected dried over anhydrous sodium sulfate, and the solvent was removed under reduced pressure to give a residue. The residue was purified silica gel chromatography (DCM/MeOH=100/0 to 15/1) to give the title compound (2 g, 30% yield) as brown solid. LC-MS (ESI⁺) m/z 545.3 (M+H)⁺.

Step 2 (S)-2-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl 4-[2-[(10R)-4-(2-hydroxyphenyl)-1,5,6,8,12-pentazatricyclo[8.4.0.0^{2,7}]tetradeca-2(7),3,5-trien-12-yl]pyrimidin-5-yl]piperidine-1-carboxylate (600 mg, 1.10 mmol) in DCM (15 mL) was added HCl/dioxane (4 M, 5 mL). The mixture was stirred at 25° C. for 3 hours. On completion, the reaction mixture was concentrated under reduced pressure to give title compound (400 mg) as a yellow solid. LC-MS (ESI⁺) m/z 445.1 (M+H)⁺.

### (2S,4R)-1-((S)-2-(1-fluorocyclopropanecarboxamido)-3,3-dimethylbutanoyl)-4-hydroxy-N-(4-(4-methylthiazol-5-yl)-2-((4-oxocyclohexyl)oxy)benzyl)pyrrolidine-2-carboxamide (Intermediate N)

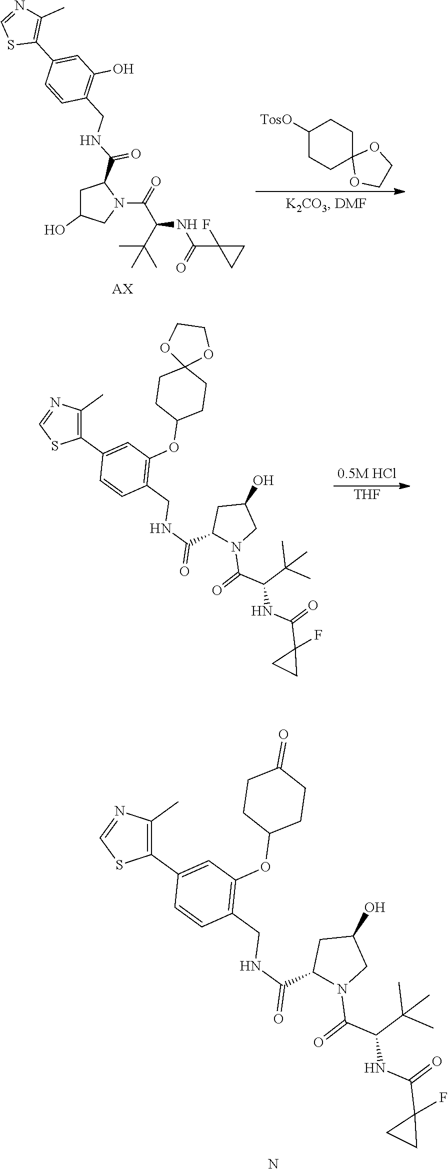

Step 1-(2S,4R)—N-(2-(1,4-dioxaspiro[4.5]decan-8-yloxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide. To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (300 mg, 563 umol, Intermediate AX) in DMF (4 mL) was added K₂CO₃ (233 mg, 1.69 mmol) and 1,4-dioxaspiro[4.5]decan-8-yl 4-methylbenzenesulfonate (264 mg, 845 umol, CAS #23511-05-9). The mixture was stirred at 70° C. for 12 hours. On completion, the reaction mixture was filtered and the filtration was concentrated under reduced pressure to give a residue. The residue was purified by prep-HPLC (FA condition) to give the title compound (150 mg, 32% yield) as a white solid. LC/MS (ESI, m/z): [M+1]⁺=673.3.

Step 2-(2S,4R)-1-((S)-2-(1-fluorocyclopropanecarboxamido)-3,3-dimethylbutanoyl)-4-hydroxy-N-(4-(4-methylthiazol-5-yl)-2-((4-oxocyclohexyl)oxy)benzyl)pyrrolidine-2-carboxamide. To a solution of (2S,4R)—N-(2-(1,4-dioxaspiro[4.5]decan-8-yloxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (100 mg, 149 umol) in THF (1 mL) was added HCl (0.5 M, 1 mL). The mixture was then stirred at 50° C. for 12 hours. On completion, the reaction mixture was concentrated under reduced pressure to give a residue. The residue was purified by reversed phase flash (FA condition) to give the title compound (80 mg, 85% yield) as a white solid. LC/MS (ESI, m/z): [M+1]+=629.5.

### 2-(3-(4-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)ethyl)piperidin-1-yl)isoxazol-5-yl)-3-methylbutanoic acid (Intermediate P)

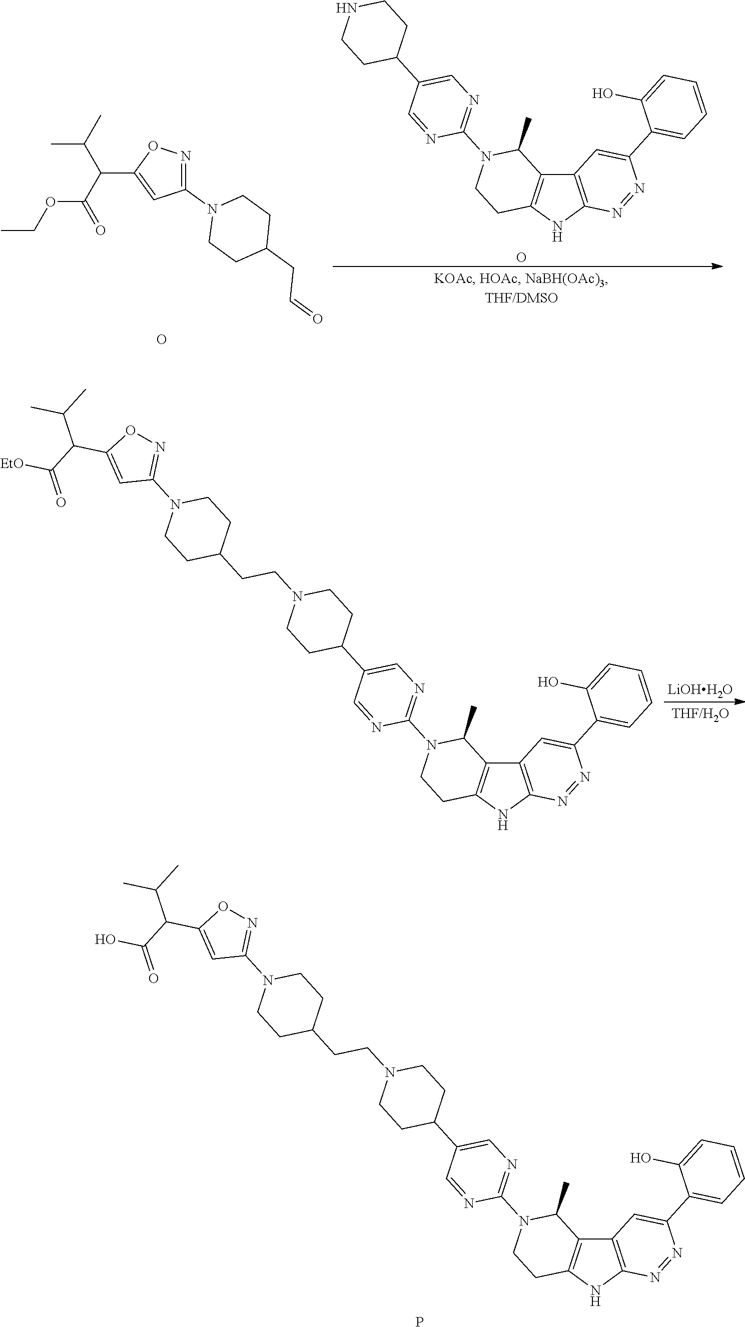

Step 1-Ethyl 2-(3-(4-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)ethyl)piperidin-1-yl)isoxazol-5-yl)-3-methylbutanoate. To a solution of ethyl 3-methyl-2-[3-[4-(2-oxoethyl)-1-piperidyl]isoxazol-5-yl]butanoate (230 mg, 713 umol, Intermediate I) in THF (5 mL) and DMSO (1 mL) was added AcOK (210 mg, 2.14 mmol), and the reaction was stirred for 30 minutes at 0° C. Then 2-[(3S)-3-methyl-4-[5-(4-piperidyl)pyrimidin-2-yl]-4,8,10,11-tetrazatricyclo[7.4.0.02,7]trideca-1(9),2(7),10,12-tetraen-12-yl]phenol (252 mg, 571 umol, Intermediate O), AcOH (128 mg, 2.14 mmol) and 4 Å molecular sieves (300 mg) was added, and the reaction was stirred at 0° C. for 30 minutes. Next, NaBH(OAc)₃ (378 mg, 1.80 mmol) was added, and the resulting solution was stirred at 0° C. for 2 hours. On completion, the reaction mixture was quenched with MeOH (5 mL), and then extracted with EA (10 mL×3). The combined organic layers were washed with brine (10 mL×3), dried over with Na₂SO₄, filtered and concentrated under reduced pressure to give a residue. The residue was purified by column chromatography (SiO₂, DCM:MeOH=10/1 to 2/1) to give the title compound (210 mg, 38% yield) as a yellow oil. LC-MS (ESI⁺) m/z 748.7 (M+H)⁺.

Step 2-2-(3-(4-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)ethyl)piperidin-1-yl)isoxazol-5-yl)-3-methylbutanoic acid. To a solution of ethyl 2-[3-[4-[2-[4-[2-[(3R)-12-(2-hydroxyphenyl)-3-methyl-4,8,10,11-tetrazatricyclo[7.4.0.02,7]trideca-1(9),2(7),10,12-tetraen-4-yl]pyrimidin-5-yl]-1-piperidyl]ethyl]-1-piperidyl]isoxazol-5-yl]-3-methyl-butanoate (200 mg, 267 umol) in a mixture solution of H₂O (3 mL), MeOH (3 mL) and THF (3 mL) was added LiOH·H₂O (56.1 mg, 1.34 mmol). After the mixture was stirred at 25° C. for 2 hours, the reaction mixture was concentrated under reduced pressure to remove most of the organic solvents. The residue was dried by lyophilization to give the title compound (230 mg) as a yellow solid. LC-MS (ESI⁺) m/z 720.6 (M+H)⁺.

### Ethyl 3-methyl-2-(3-(((perfluorobutyl)sulfonyl)oxy)isoxazol-5-yl)butanoate (Intermediate Q)

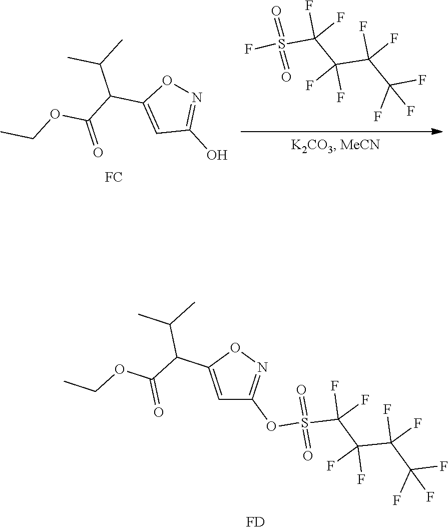

To a solution of ethyl 2-(3-hydroxyisoxazol-5-yl)-3-methyl-butanoate (3 g, 14.1 mmol, Intermediate FC) in MeCN (20 mL) was added 1,1,2,2,3,3,4,4,4-nonafluorobutane-1-sulfonyl fluoride (4.68 g, 15.5 mmol, 2.72 mL) and K₂CO₃ (3.89 g, 28.1 mmol). The mixture was stirred at 40° C. for 8 hours. On completion, the mixture was concentrated under reduced pressure to give a residue. The residue was purified by column chromatography (SiO₂, Petroleum ether/Ethyl acetate=1/0 to 10/1) to give the title compound (5.2 g, 71% yield) as a colorless oil. LC-MS (ESI⁺) m/z 496.2 (M+H)⁺.

### Ethyl 3-methyl-2-(3-(7-oxo-2-azaspiro[3.5]nonan-2-yl)isoxazol-5-yl)butanoate (Intermediate R)

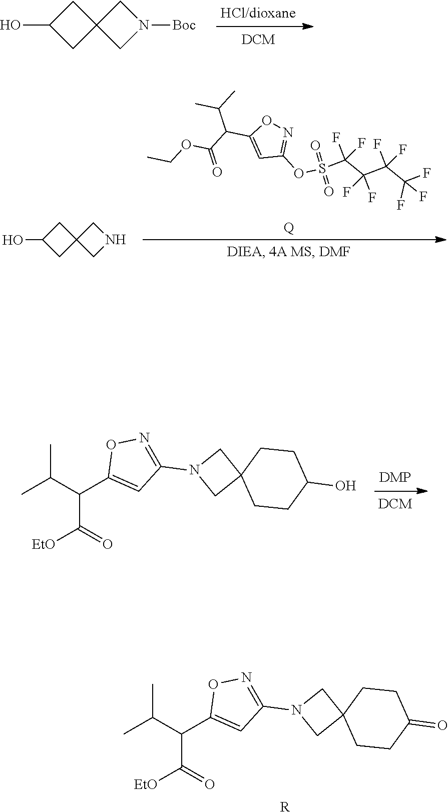

Step 1-2-Azaspiro[3.5]nonan-7-ol. To a solution of tert-butyl 7-hydroxy-2-azaspiro[3.5]nonane-2-carboxylate (1 g, 4.14 mmol, CAS #1363383-18-9) in DCM (100 mL) was added HCl/dioxane (4 M, 3.11 mL). The mixture was stirred at 25° C. for 12 hours. On completion, the reaction mixture was concentrated in vacuo to give the title compound (780 mg). ¹H NMR (400 MHz, DMSO-d₆) δ=9.46-9.20 (m, 2H), 3.69-3.54 (m, 4H), 3.43-3.32 (m, 1H), 1.98-1.87 (m, 2H), 1.66-1.55 (m, 2H), 1.51-1.39 (m, 2H), 1.23-1.11 (m, 2H).

Step 2-Ethyl 2-(3-(7-hydroxy-2-azaspiro[3.5]nonan-2-yl)isoxazol-5-yl)-3-methylbutanoate. To a solution of ethyl 3-methyl-2-(3-(((perfluorobutyl)sulfonyl)oxy)isoxazol-5-yl)butanoate (1.8 g, 3.36 mmol, Intermediate Q) in DMF (4 mL) was added 2-azaspiro[3.5]nonan-7-ol (770 mg, 5.45 mmol), DIEA (1.88 g, 14.5 mmol, 2.53 mL) and 4 Å molecular sieves (2 g, 3.63 mmol). Then the mixture was stirred at 130° C. for 2 hours. On completion, the reaction mixture was filtered and concentrated under reduced pressure to give a residue. The crude product was purified by reversed-phase HPLC (0.1% FA condition) to give the title compound (820 mg, 58% yield) as yellow oil. LC-MS (ESI⁺) m/z 337.1 (M+H)⁺.

Step 3-Ethyl 3-methyl-2-(3-(7-oxo-2-azaspiro[3.5]nonan-2-yl)isoxazol-5-yl)butanoate. To a solution of DMP (1.31 g, 3.09 mmol) in DCM (8 mL) was added ethyl 2-(3-(7-hydroxy-2-azaspiro[3.5]nonan-2-yl)isoxazol-5-yl)-3-methylbutanoate (800 mg, 2.38 mmol) at 0° C. Then the mixture was stirred at 20° C. for 2 hours. On completion, the reaction mixture was quenched with NaS₂O₃ aqueous solution (10 mL) and saturated NaHCO₃ aqueous solution (10 mL) at 25° C. Then the mixture was extracted with DCM (3×30 mL), dried over Na₂SO₄, filtered and concentrated under reduced pressure to give a residue. The crude product was purified by reversed-phase HPLC (0.1% FA condition) to give the title compound (370 mg, 46% yield) as yellow oil. 1H NMR (400 MHz, DMSO-d6) δ=5.97-5.95 (m, 1H), 4.23-4.04 (m, 2H), 3.58 (d, J=8.8 Hz, 1H), 3.35-3.29 (m, 1H), 2.51 (s, 3H), 2.32-2.21 (m, 5H), 2.09-2.00 (m, 4H), 1.22-1.17 (m, 3H), 0.94 (d, J=6.6 Hz, 3H), 0.85 (d, J=6.8 Hz, 3H) LC-MS (ESI⁺) m/z 335.8 (M+H)⁺.

### 2-(3-(7-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.5]nonan-2-yl)isoxazol-5-yl)-3-methylbutanoic acid (Intermediate S)

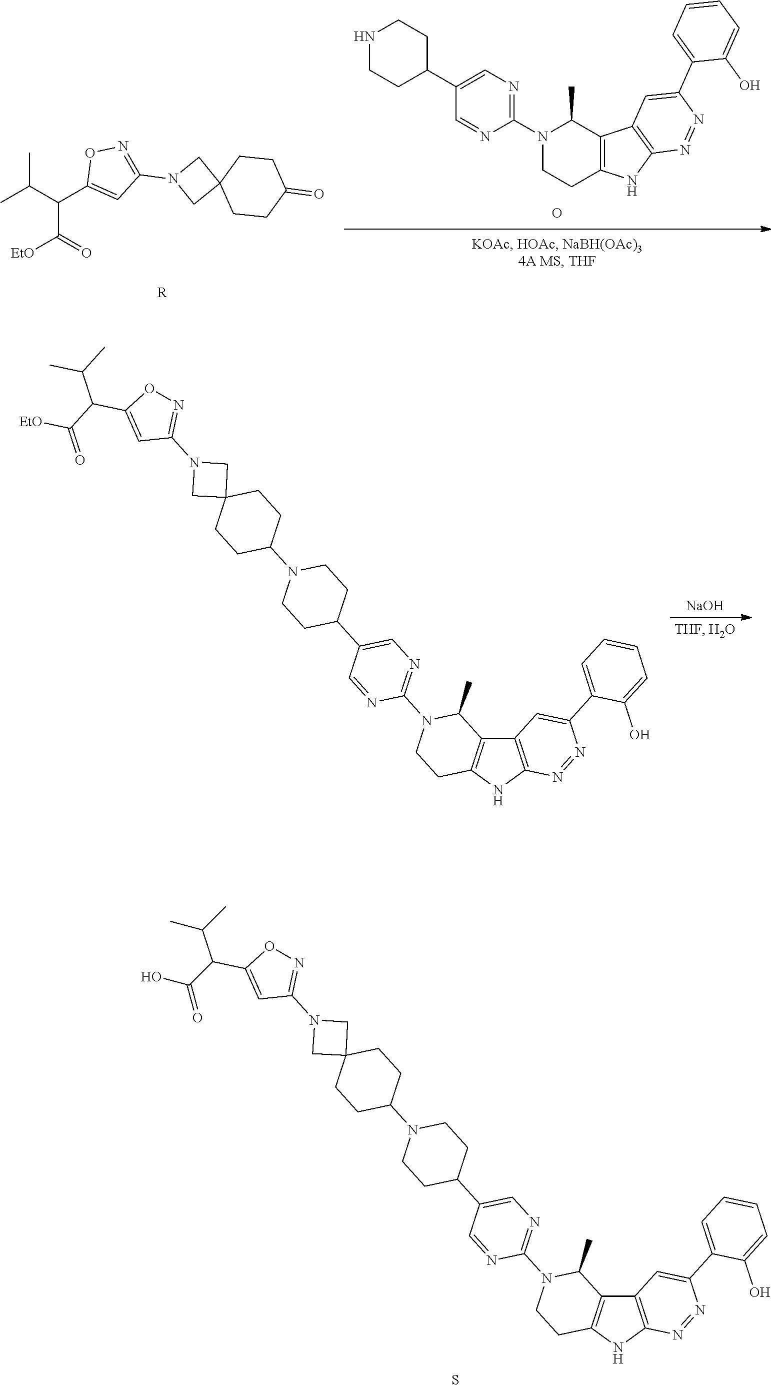

Step 1-Ethyl 2-(3-(7-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.5]nonan-2-yl)isoxazol-5-yl)-3-methylbutanoate. To a solution of (S)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (184 mg, 411 umol, HCl, Intermediate O) in THF (4 mL) was added KOAc (40.4 mg, 411 mmol), AcOH (75.4 mg, 1.26 mmol, 71.8 uL) and 4 Å molecular sieves (200 mg) stirred at 25° C. for 10 min. Then ethyl 3-methyl-2-(3-(7-oxo-2-azaspiro[3.5]nonan-2-yl)isoxazol-5-yl)butanoate (165 mg, 493 umol, Intermediate R) was added to the mixture and the reaction was stirred at 25° C. for 1.5 h. Next, NaBH(OAc)₃ (218 mg, 1.03 mmol) was added at 0° C., then the mixture was stirred at 25° C. for 2 hours. On completion, the reaction mixture was filtered and concentrated under reduced pressure to give a residue. The crude product was purified by reversed-phase HPLC (0.1% FA condition) to give the title compound (40 mg, 13% yield) as yellow solid. LC-MS (ESI⁺) m/z 760.5 (M+H)⁺.

Step 2-2-(3-(7-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.5]nonan-2-yl)isoxazol-5-yl)-3-methylbutanoic acid. To a solution of ethyl 2-(3-(7-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.5]nonan-2-yl)isoxazol-5-yl)-3-methylbutanoate (35 mg, 46.1 umol) in THF (1 mL) and H₂O (0.5 mL) was added NaOH (9.21 mg, 230 umol). Then the mixture was stirred at 25° C. for 2 hours. On completion, the reaction mixture was concentrated in vacuo to give the title compound (250 mg). LC-MS (ESI⁺) m/z 732.4 (M+H)⁺.

### 2-(3-(7-(4-(2-((R)-3-(2-hydroxyphenyl)-5-methyl-5,7,8,9-tetrahydro-6H-pyrido[3′,4′:4,5]pyrrolo[2,3-c]pyridazin-6-yl)pyrimidin-5-yl)piperidin-1-yl)-2-azaspiro[3.5]nonan-2-yl)isoxazol-5-yl)-3-methylbutanoic acid (Intermediate T)

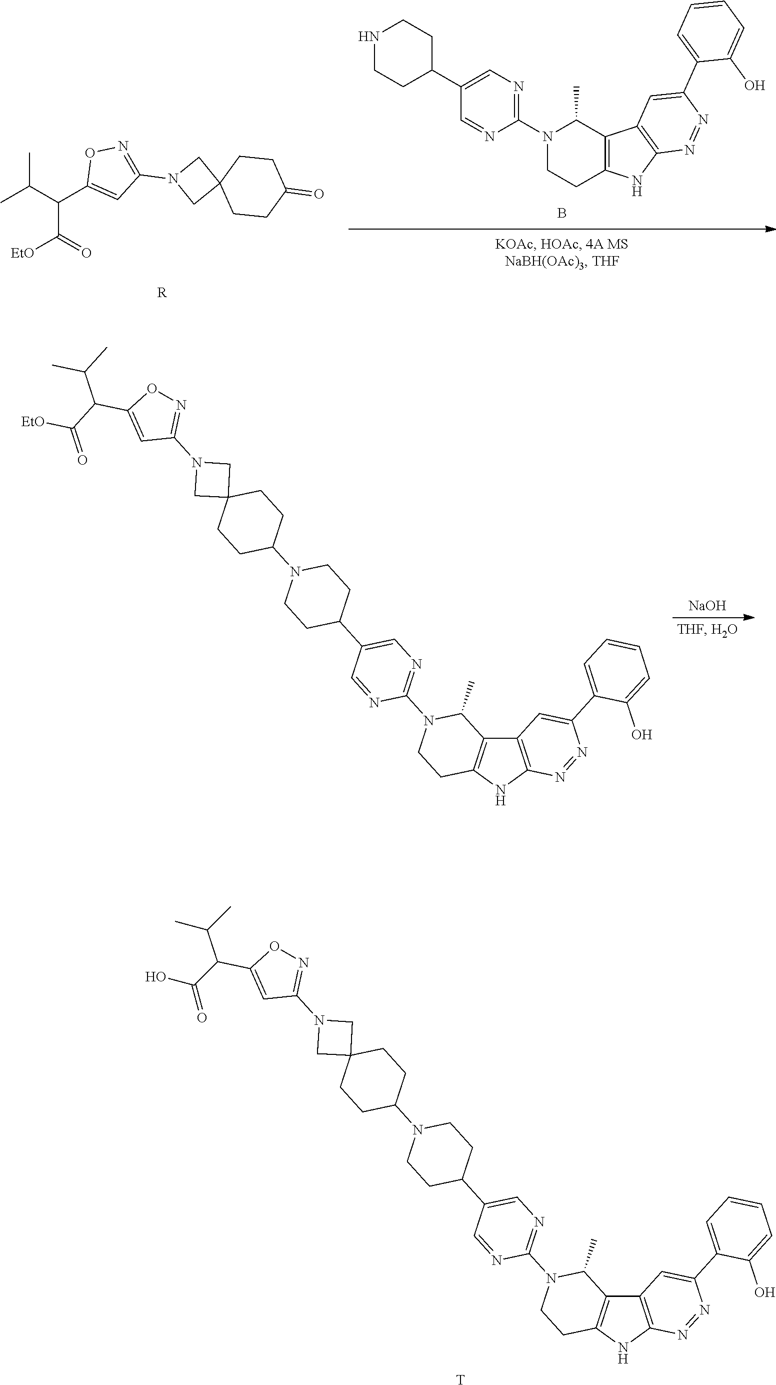

Step 1-Ethyl 2-(3-(7-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.5]nonan-2-yl)isoxazol-5-yl)-3-methylbutanoate. To a solution of (R)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (172 mg, 359 umol, HCl, Intermediate B) in THF (6 mL) and DMSO (0.5 mL) was added KOAc (114 mg, 1.17 mmol), AcOH (70.0 mg, 1.17 mmol, 66.7 uL) and 4 Å molecular sieves (300 mg) and the mixture was stirred at 25° C. for 10 min. Then ethyl 3-methyl-2-(3-(7-oxo-2-azaspiro[3.5]nonan-2-yl)isoxazol-5-yl)butanoate (130 mg, 389 umol, Intermediate R) was added and the mixture was stirred at 25° C. for 1.5 h. Next, NaBH(OAc)₃ (206 mg, 912 umol) was added at 0° C., then the mixture was stirred at 25° C. for 2 hours. On completion, the reaction mixture was filtered and concentrated under reduced pressure to give a residue. The crude product was purified by reversed-phase HPLC (0.1% FA condition) to give the title compound (45 mg, 15% yield) as a yellow solid. LC-MS (ESI⁺) m/z 760.4 (M+H)⁺.

Step 2-2-(3-(7-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.5]nonan-2-yl)isoxazol-5-yl)-3-methylbutanoic acid. To a solution of ethyl 2-(3-(7-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.5]nonan-2-yl)isoxazol-5-yl)-3-methylbutanoate (45 mg, 59.2 umol) in THF (2 mL) and H₂O (0.5 mL) was added NaOH (11.8 mg, 296 umol). The mixture was stirred at 25° C. for 2 hours. On completion, the reaction mixture was concentrated in vacuo to give the title compound (110 mg). LC-MS (ESI⁺) m/z 732.3 (M+H)⁺.

### Ethyl 3-methyl-2-(3-(2-oxo-7-azaspiro[3.5]nonan-7-yl)isoxazol-5-yl)butanoate (Intermediate U)

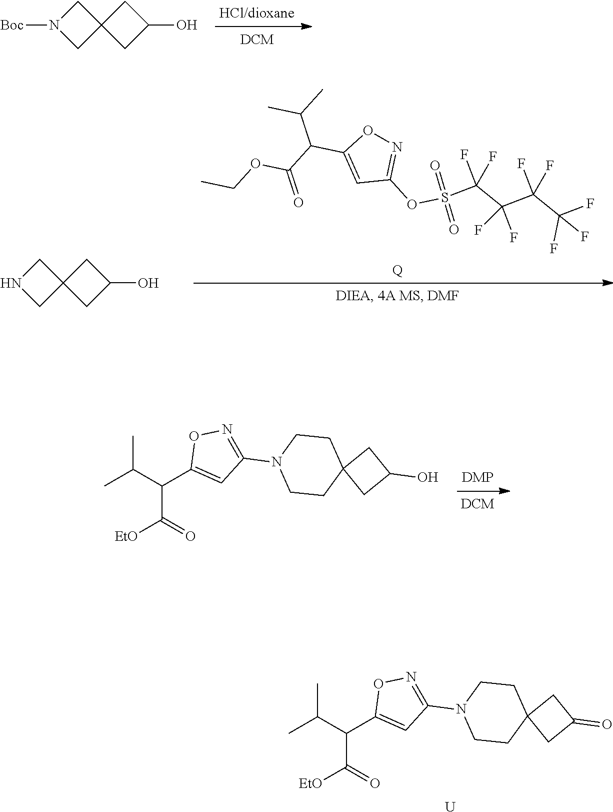

Step 1-7-Azaspiro[3.5]nonan-2-ol. To a solution of tert-butyl 2-hydroxy-7-azaspiro[3.5]nonane-7-carboxylate (2 g, 8.29 mmol, CAS #240401-28-9) in DCM (20 mL) was added HCl/dioxane (4 M, 6.22 mL). The mixture was stirred at 25° C. for 12 hours. On completion, the reaction mixture was concentrated in vacuo to give the title compound (1.8 g). ¹H NMR (400 MHz, DMSO-d₆) δ=9.12-8.71 (m, 2H), 4.07 (quin, J=7.2 Hz, 1H), 2.97-2.84 (m, 4H), 2.23-2.10 (m, 2H), 1.71-1.48 (m, 6H).

Step 2-Ethyl 2-(3-(2-hydroxy-7-azaspiro[3.5]nonan-7-yl)isoxazol-5-yl)-3-methylbutanoate. To a solution of ethyl 3-methyl-2-(3-(((perfluorobutyl)sulfonyl)oxy)isoxazol-5-yl)butanoate (1.17 g, 2.36 mmol, Intermediate Q) in DMF (10 mL) was added 7-azaspiro[3.5]nonan-2-ol (500 mg, 3.54 mmol), DIEA (1.22 g, 9.44 mmol, 1.64 mL) and 4 Å molecular sieves (2.5 g, 2.36 mmol). The mixture was stirred at 130° C. for 1.5 hours. On completion, the reaction mixture was diluted with H₂O (10 mL) and extracted with EA (3×10 mL). The combined organic layers were washed with brine, dried over Na₂SO₄, filtered and concentrated under reduced pressure to give a residue. The residue was purified by column chromatography (SiO₂, Petroleum ether/Ethyl acetate=1/1) to give the title compound (500 mg, 59% yield) as brown solid. LC-MS (ESI⁺) m/z 337.8 (M+H)⁺.

Step 3-Ethyl 3-methyl-2-(3-(2-oxo-7-azaspiro[3.5]nonan-7-yl)isoxazol-5-yl)butanoate. To a solution of DMP (738 mg, 1.74 mmol) in DCM (4 mL) was added ethyl 2-(3-(2-hydroxy-7-azaspiro[3.5]nonan-7-yl)isoxazol-5-yl)-3-methylbutanoate (450 mg, 1.34 mmol) at 0° C. The mixture was stirred at 20° C. for 4 hours. On completion, the reaction mixture was with saturated NaS₂O₃ aqueous solution (10 mL) and saturated NaHCO₃ aqueous solution (10 mL) at 25° C. Then the mixture was extracted with DCM (3×30 mL), dried over Na₂SO₄, filtered and concentrated under reduced pressure to give a residue. The crude product was purified by reversed-phase HPLC (0.1% FA condition) to give the title compound (30 mg, 16% yield) as yellow oil. ¹H NMR (400 MHz, DMSO-d₆) δ=6.31-6.23 (m, 1H), 4.21-4.05 (m, 2H), 3.58-3.50 (m, 1H), 3.24-3.16 (m, 4H), 2.90-2.78 (m, 4H), 2.37-2.19 (m, 1H), 1.79-1.67 (m, 4H), 1.24-1.15 (m, 3H), 1.00-0.90 (m, 3H), 0.89-0.80 (m, 3H). LC-MS (ESI⁺) m/z 335.7 (M+H)⁺.

### 2-(3-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonan-7-yl)isoxazol-5-yl)-3-methylbutanoic acid (Intermediate V)

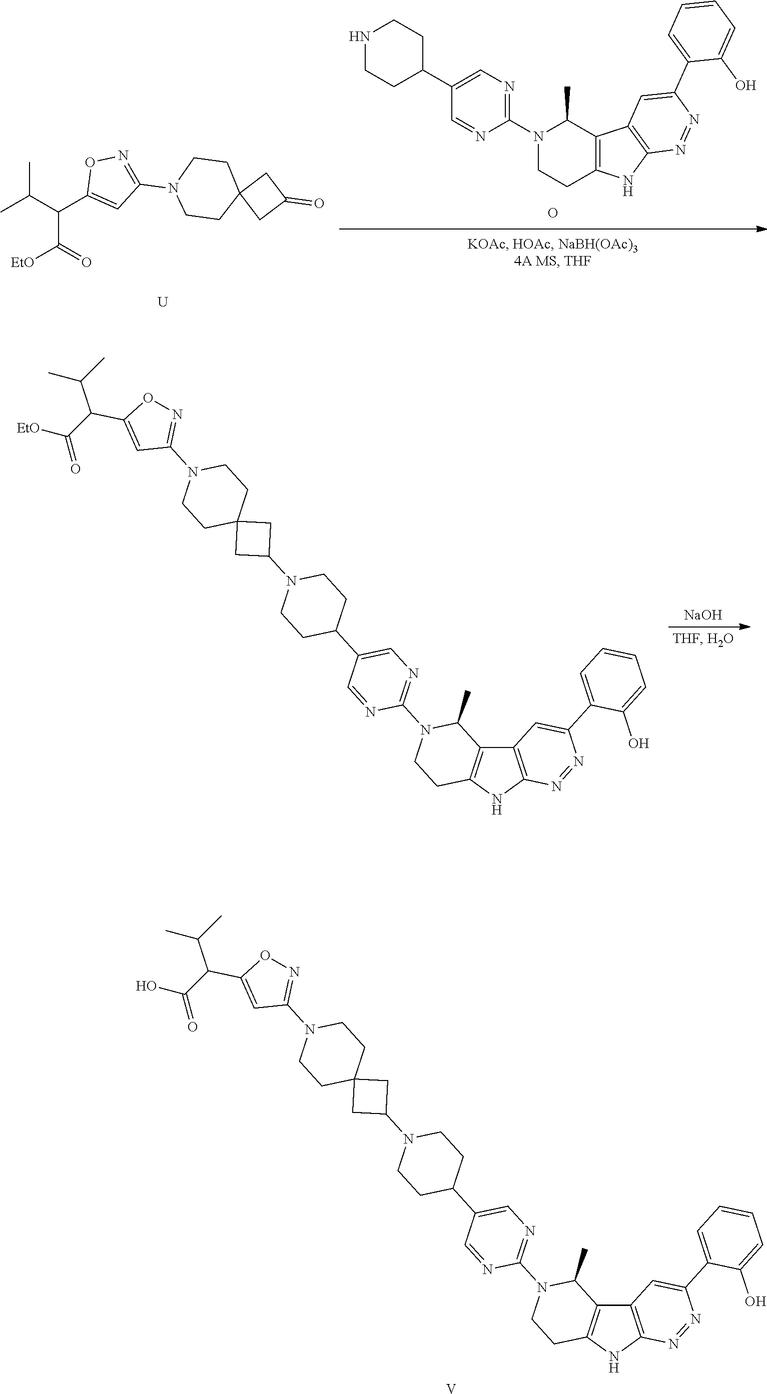

Step 1-Ethyl 2-(3-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonan-7-yl)isoxazol-5-yl)-3-methylbutanoate. To a solution of (S)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (185 mg, 418 umol, HCl, Intermediate O) in THF (2 mL) was added KOAc (123 mg, 1.26 mmol), AcOH (75.4 mg, 1.26 mmol, 71.8 uL) and 4 Å molecular sieves (200 mg) and the mixture was stirred at 25° C. for 10 min. Then ethyl 3-methyl-2-[3-(2-oxo-7-azaspiro[3.5]nonan-7-yl)isoxazol-5-yl]butanoate (140 mg, 418 umol, Intermediate U) was added and the mixture was stirred at 25° C. for 1.5 h. Next, NaBH(OAc)₃ (222 mg, 1.05 mmol) was added at 0° C. and the mixture was stirred at 25° C. for 2 hours. On completion, the reaction mixture was filtered and concentrated under reduced pressure to give a residue. The crude product was purified by reversed-phase HPLC (0.1% FA condition) to give the title compound (100 mg, 29% yield) as yellow solid. LC-MS (ESI⁺) m/z 760.4 (M+H)⁺.

Step 2-2-(3-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonan-7-yl)isoxazol-5-yl)-3-methylbutanoic acid. To a solution of ethyl 2-(3-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonan-7-yl)isoxazol-5-yl)-3-methylbutanoate (80.0 mg, 105 umol) in THF (2 mL) and H₂O (0.5 mL) was added NaOH (4.21 mg, 105 umol). Then the mixture was stirred at 25° C. for 2 hours. On completion, the reaction mixture was concentrated in vacuo to give the title compound (350 mg). LC-MS (ESI⁺) m/z 732.6 (M+H)⁺.

### 2-(3-(2-(4-(2-((R)-3-(2-hydroxyphenyl)-5-methyl-5,7,8,9-tetrahydro-6H-pyrido[3′,4′:4,5]pyrrolo[2,3-c]pyridazin-6-yl)pyrimidin-5-yl)piperidin-1-yl)-7-azaspiro[3.5]nonan-7-yl)isoxazol-5-yl)-3-methylbutanoic acid (Intermediate W)

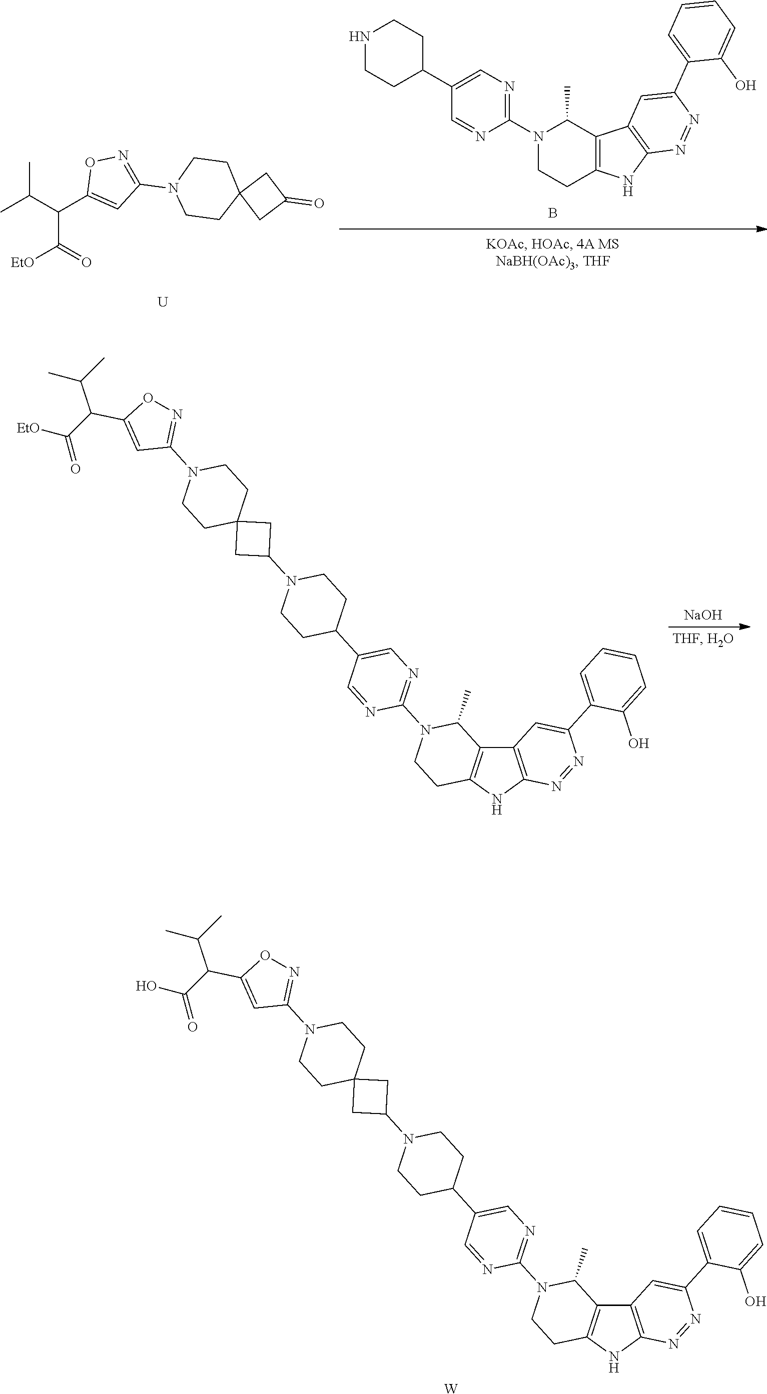

Step 1-Ethyl 2-(3-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonan-7-yl)isoxazol-5-yl)-3-methylbutanoate. To a solution of ((R)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (186 mg, 389 umol, HCl, Intermediate B) in THF (3 mL) and DMSO (1 mL) was added KOAc (114 mg, 1.17 mmol). Then the reaction was stirred at 25° C. for 10 min. Next, 4 Å molecular sieves (200 mg), AcOH (70.0 mg, 1.17 mmol) and ethyl 3-methyl-2-(3-(2-oxo-7-azaspiro[3.5]nonan-7-yl)isoxazol-5-yl)butanoate (130 mg, 389 umol, Intermediate U) was added to the mixture and the mixture was stirred at 25° C. for 12 hours. Finally, NaBH(OAc)₃ (206 mg, 972 umol) was added to the mixture at 0° C., then the mixture was stirred at 25° C. for 3 hr. On completion, the reaction mixture was filtered and concentrated under reduced pressure to give a residue. The crude product was purified by reversed-phase HPLC (0.1% FA condition) to give the title compound (50 mg, 14% yield) as a yellow solid. LC-MS (ESI⁺) m/z 760.4 (M+H)⁺.

Step 2-2-(3-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonan-7-yl)isoxazol-5-yl)-3-methylbutanoic acid. To a solution of ethyl 2-(3-(2-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonan-7-yl)isoxazol-5-yl)-3-methylbutanoate (50.0 mg, 65.8 umol) in THF (2 mL) and H₂O (0.5 mL) was added NaOH (13.2 mg, 329 umol). The mixture was then stirred at 25° C. for 2 hours. On completion, the reaction mixture was concentrated in vacuo to give the title compound (50 mg). LC-MS (ESI⁺) m/z 732.3 (M+H)⁺.

### (R)-2-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate X)

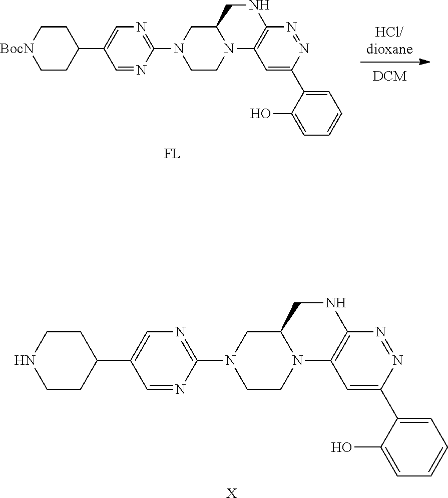

To a solution of tert-butyl 4-[2-[(1OR)-4-(2-hydroxyphenyl)-1,5,6,8,12-pentazatricyclo[8.4.0.0^{2,7}]tetradeca-2(7),3,5-trien-12-yl]pyrimidin-5-yl]piperidine-1-carboxylate (600 mg, 1.10 mmol, Intermediate FL) in DCM (15 mL) was added HCl/dioxane (4 M, 5 mL). The mixture was stirred at 25° C. for 3 hours. On completion, the reaction mixture was concentrated under reduced pressure to give title compound (400 mg) as a yellow solid. LC-MS (ESI⁺) m/z 445.1 (M+H)⁺.

### 3-(2-(methoxymethoxy)phenyl)-5-methyl-6,7,8,9-tetrahydro-5H-pyrido[3′,4′:4,5]pyrrolo[2,3-c]pyridazine (Intermediate Y)

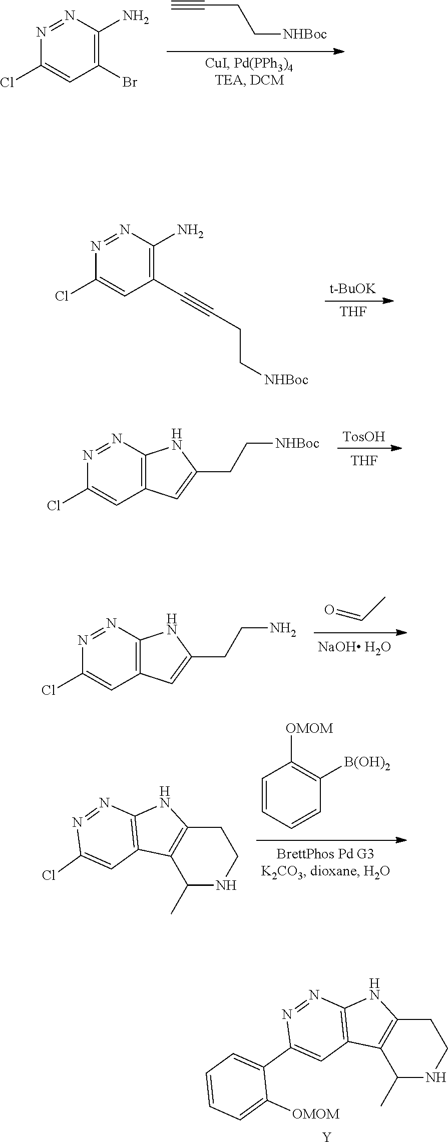

Step 1-tert-butyl (4-(3-amino-6-chloropyridazin-4-yl)but-3-yn-1-yl)carbamate. A mixture of 4-bromo-6-chloro-pyridazin-3-amine (80 g, 380 mmol, CAS #446273-59-2), tert-butyl N-but-3-ynylcarbamate (94.00 g, 555.49 mmol, CAS #149990-27-2), TEA (388.36 g, 3.84 mol), Pd(PPh₃)₄(22.18 g, 19.19 mmol) and CuI (7.31 g, 38.38 mmol) in DMF (1200 mL) was degassed and purged with N₂ three times. Then the mixture was stirred at 35° C. for 2 hours under N₂ atmosphere. On completion, the reaction mixture was partitioned between ethyl acetate (600 mL) and water (500 mL). The organic phase was separated, washed with brine (250 mL×2), dried over Na₂SO₄, filtered and concentrated under reduced pressure to give a residue. The residue was purified by flash silica gel chromatography (SiO₂, Petroleum ether/Ethyl acetate=10/1 to 1/1) to give the title compound (102 g, 81% yield) as a brown solid. LC/MS (ESI, m/z): [M+1]⁺=297.3.

Step 2-tert-butyl (2-(3-chloro-7H-pyrrolo[2,3-c]pyridazin-6-yl)ethyl)carbamate. To a solution of tert-butyl N-[4-(3-amino-6-chloro-pyridazin-4-yl)but-3-ynyl]carbamate (102 g, 344 mmol) in THF (1000 mL) was added t-BuOK (46.3 g, 412 mmol). The mixture was stirred at 0-25° C. for 2 hours. On completion, the reaction mixture was quenched with saturated NH₄Cl aqueous solution (800 mL) at 0° C., then diluted with ethyl acetate (2000 mL) and extracted with water (800 mL×2). The combined organic layers were washed with brine (500 mL×2), filtered and concentrated under reduced pressure to give a residue. The crude product was triturated with petroleum ether/ethyl acetate=1/1 at 25° C. for 20 min to give the title compound (84 g, 81% yield) as a brown solid. ¹H NMR (400 MHz, DMSO-d₆) δ=7.83 (s, 1H), 7.00 (t, J=5.6 Hz, 1H), 6.30 (s, 1H), 3.34 (q, J=6.4 Hz, 3H), 2.94 (t, J=6.8 Hz, 2H), 1.33 (s, 9H). LC/MS (ESI, m/z): [M+1]⁺=296.9.

Step 3-2-(3-chloro-7H-pyrrolo[2,3-c]pyridazin-6-yl)ethanamine. To a solution of tert-butyl N-[2-(3-chloro-7H-pyrrolo[2,3-c]pyridazin-6-yl)ethyl]carbamate (79 g, 266 mmol) in THF (800 mL) was added TosOH (91.7 g, 532 mmol). The mixture was stirred at 70° C. for 12 hours. The reaction mixture was filtered and concentrated under reduced pressure to give the crude compound (54 g) as a brown solid. LC/MS (ESI, m/z): [M+1]+=197.1.

Step 4-3-chloro-5-methyl-6,7,8,9-tetrahydro-5H-pyrido[3′,4′:4,5]pyrrolo[2,3-c]pyridazine. To a solution of 2-(3-chloro-7H-pyrrolo[2,3-c]pyridazin-6-yl)ethanamine (54 g, 275 mmol) and acetaldehyde (36.3 g, 824 mmol) was added in H₂O (400 mL). The mixture was stirred at 70° C. for 12 hours. On completion, the reaction mixture was filtered and concentrated under reduced pressure to give a residue. The crude product was purified by reversed-phase HPLC (0.1% NH₃—H₂O condition) to give the title compound (55 g, 84% yield) as a brown solid. LC/MS (ESI, m/z): [M+1]⁺=223.1.

Step 5-3-(2-(methoxymethoxy)phenyl)-5-methyl-6,7,8,9-tetrahydro-5H-pyrido[3′,4′:4,5]pyrrolo[2,3-c]pyridazine. A mixture of [2-(methoxymethoxy)phenyl]boronic acid (8.17 g, 44.9 mmol, CAS #115377-93-0), 12-chloro-3-methyl-4,8,10,11-tetrazatricyclo[7.4.0.02,7]trideca-1(9),2(7),10,12-tetraene (5 g, 22.4 mmol), BrettPhos Pd G3 (2.04 g, 2.25 mmol), K₂CO₃ (9.31 g, 67.4 mmol) in dioxane (50 mL) and H₂O (10 mL) was degassed and purged with nitrogen three times. Then the mixture was stirred at 80° C. for 12 hours under N₂ atmosphere. On completion, the reaction mixture was partitioned between ethyl acetate (300 mL) and water (200 mL). The organic phase was separated, washed with brine (100 mL×2) and dried over Na2SO₄, filtered and concentrated under reduced pressure to give a residue. The residue was purified by column chromatography (SiO₂, DCM: MeOH=100:1 to 10:1) to give the title compound (4 g, 49% yield) as a yellow solid. LC/MS (ESI, m/z): [M+1]⁺=325.1.

### 2-(5-Methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (Intermediate Z)

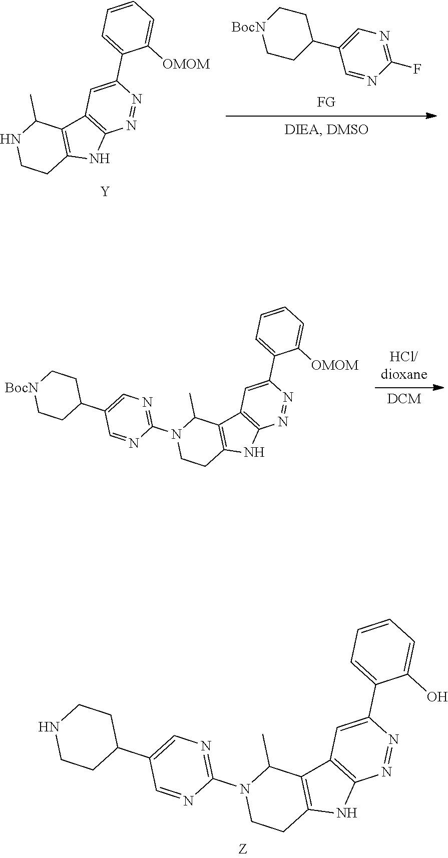

Step 1-Tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidine-1-carboxylate. To a solution of 12-[2-(methoxymethoxy)phenyl]-3-methyl-4,8,10,11-tetrazatricyclo[7.4.0.0^{2,7}]trideca-1(9),2(7),10,12-tetraene (5 g, 15.4 mmol, Intermediate Y) and tert-butyl 4-(2-fluoropyrimidin-5-yl)piperidine-1-carboxylate (4.34 g, 15.4 mmol, Intermediate FG) in DMSO (50 mL) was added DIEA (5.98 g, 46.2 mmol). The mixture was stirred at 80° C. for 12 hours. On completion, the reaction mixture was partitioned between ethyl acetate (300 mL) and water (200 mL). The organic phase was separated, washed with brine 200 mL (100 mL×2), dried over Na₂SO₄, filtered and concentrated under reduced pressure to give a residue. The residue was purified by column chromatography (SiO₂, DCM/MeOH=200/1 to 20/1) to give the title compound (5.4 g, 60% yield) as a yellow solid. LC/MS (ESI, m/z): [M+1]⁺=586.1.

Step 2-2-(5-Methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol. To a mixture of Tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidine-1-carboxylate (50.0 mg, 77.4 umol) in DCM (0.5 mL) was added HCl/dioxane (4 M, 0.1 mL) in one portion at 25° C. under N₂. The mixture was stirred at 25° C. for 30 minutes. On completion, the reaction mixture was concentrated in vacuo to give the title compound (50.0 mg) as a white solid. LC/MS (ESI, m/z): [M+1]⁺=442.0.

### 2-(6-(5-(1-(2-azaspiro[3.3]heptan-6-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate AA)

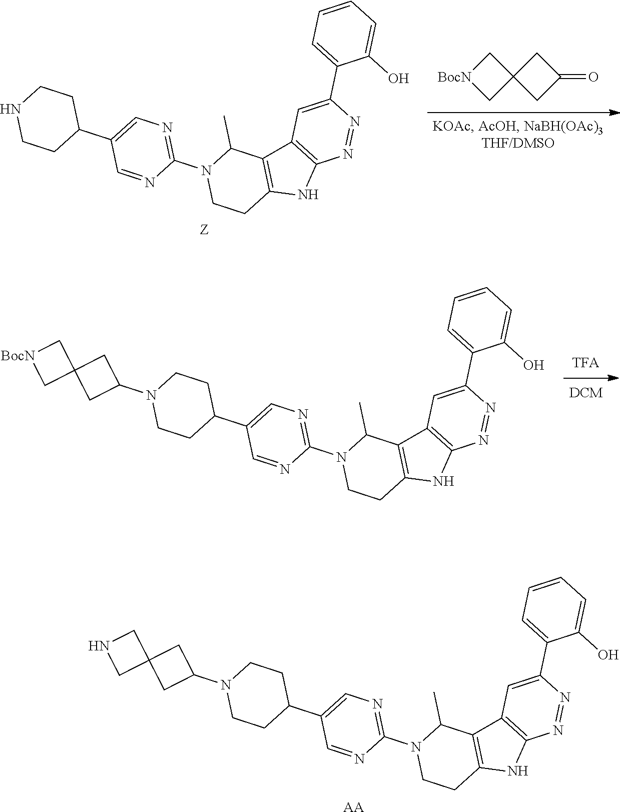 [SMILES: CC1c2c([nH]c3nnc(-c4ccccc4O)cc23)CCN1c1ncc(C2CCNCC2)cn1 ; CC(C)(C)OC(=O)N1CC2(CC(=O)C2)C1 ; CC1c2c([nH]c3nnc(-c4ccccc4O)cc23)CCN1c1ncc(C2CCN(C3CC4(C3)CN(C(=O)OC(C)(C)C)C4)CC2)cn1 ; CC1c2c([nH]c3nnc(-c4ccccc4O)cc23)CCN1c1ncc(C2CCN(C3CC4(CNC4)C3)CC2)cn1 ; "AA"]

Step 1-Tert-butyl 6-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate. To a solution of 2-[3-methyl-4-[5-(4-piperidyl)pyrimidin-2-yl]-4,8,10,11-tetrazatricyclo[7.4.0.0^{2,7}]trideca-1(9),2(7),10,12-tetraen-12-yl]phenol (6.8 g, 14.2 mmol, Intermediate Z) in THF (60 mL) and DMSO (30 mL) was added KOAc (4.19 g, 42.7 mmol) and the mixture was stirred at 25° C. for 30 minutes. Then tert-butyl 6-oxo-2-azaspiro[3.3]heptane-2-carboxylate (3.61 g, 17.1 mmol, CAS #1181816-12-5) and HOAc (3.42 g, 56.9 mmol) was added into the reaction mixture and the mixture was stirred at 0° C. for 30 minutes. Next, NaBH(OAc)₃ (9.05 g, 42.7 mmol) was added and the mixture was stirred at 0-25° C. for 3 hours. On completion, the reaction mixture was partitioned between ethyl acetate (500 mL) and water (400 mL). The organic phase was separated, dried over Na₂SO₄, filtered and concentrated under reduced pressure to give a residue. The crude product was purified by reversed-phase HPLC (0.1% FA condition) to give the title compound (8.2 g, 87% yield) as a yellow solid. LC-MS (ESI⁺) m/z 637.2 (M+H)⁺.

Step 2-2-(6-(5-(1-(2-azaspiro[3.3]heptan-6-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. A solution of tert-butyl 6-[4-[2-[12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-2-azaspiro[3.3]heptane-2-carboxylate (2 g, 3.14 mmol) in DCM (20 mL) was added TFA (4 mL). The mixture was stirred at 25° C. for 12 hours. On completion, the reaction mixture was filtered and concentrated under reduced pressure to give the title compound (2.5 g) as a yellow solid. LC-MS (ESI⁺) m/z 537.3 (M+H)⁺.

### Phenyl N-[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]carbamate (Intermediate AB)

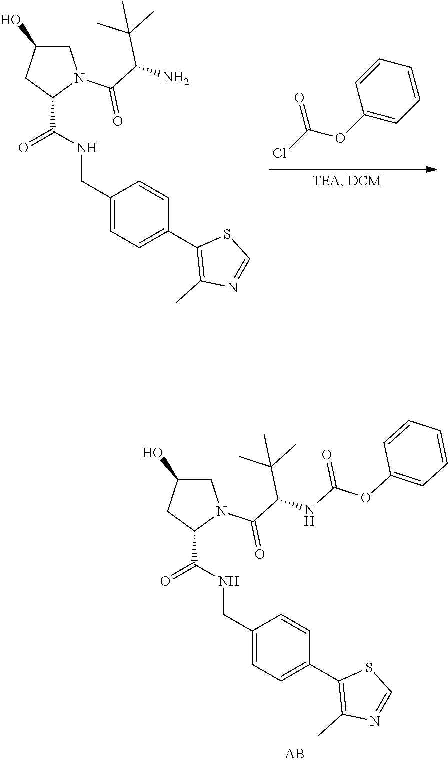 [SMILES: Cc1ncsc1-c1ccc(CNC(=O)[C@@H]2C[C@@H](O)CN2C(=O)[C@@H](N)C(C)(C)C)cc1 ; O=C(Cl)Oc1ccccc1 ; Cc1ncsc1-c1ccc(CNC(=O)[C@@H]2C[C@@H](O)CN2C(=O)[C@@H](NC(=O)Oc2ccccc2)C(C)(C)C)cc1]

To a solution of (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (5 g, 10.7 mmol, CAS #1448189-80-7) in DCM (60 mL) was added TEA (3.25 g, 32.1 mmol) and the reaction was stirred from 0-25° C. Then phenyl carbonochloridate (1.84 g, 11.8 mmol, CAS #1885-14-9) was added at 0° C., and the mixture was stirred at 25° C. for 1 hour. On completion, the reaction mixture was partitioned between DCM (500 mL) and water (400 mL). The organic phase was separated, dried over Na₂SO₄, filtered and concentrated under reduced pressure to give a residue. The residue was purified by column chromatography (SiO₂, Petroleum ether/Ethyl acetate=5/1 to 0/1) to give the title compound (3.5 g, 58% yield) as a white solid. LC-MS (ESI⁺) m/z 551.1 (M+H)⁺.

### 2-(3-(6-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptan-2-yl)isoxazol-5-yl)-3-methylbutanoic acid (Intermediate AC)

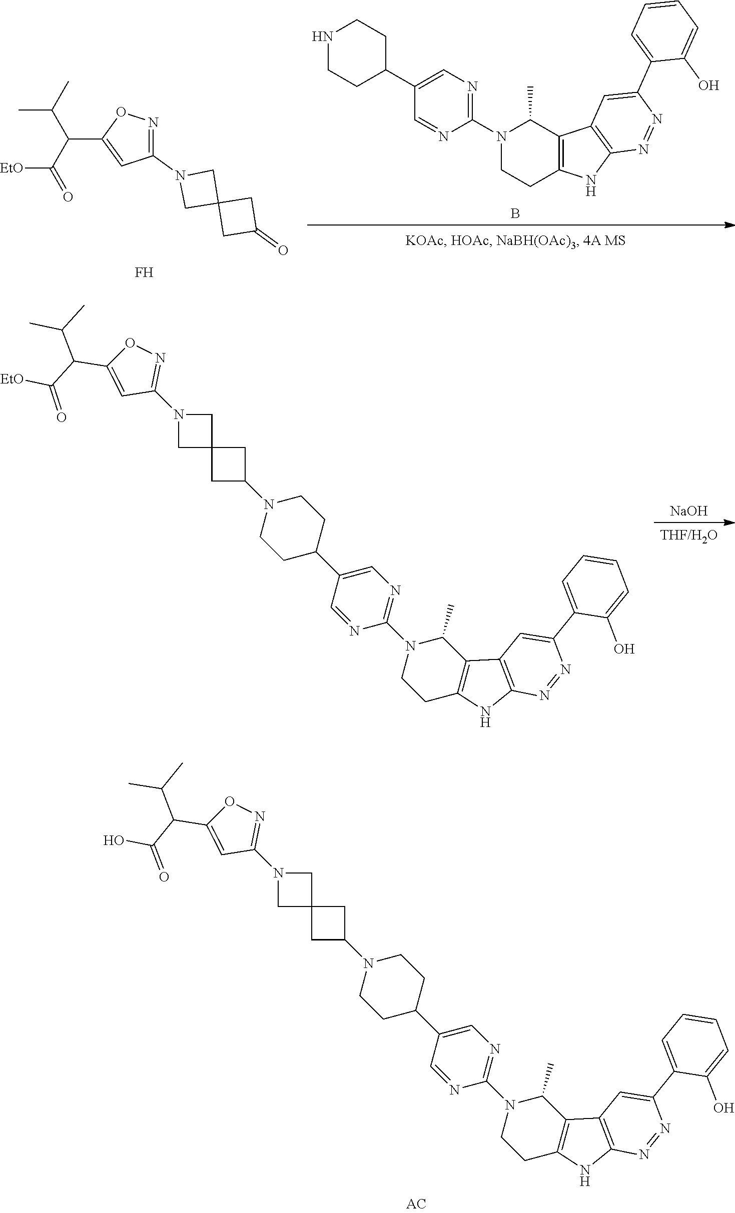

Step 1-Ethyl 2-(3-(6-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptan-2-yl)isoxazol-5-yl)-3-methylbutanoate. To a solution of ethyl 3-methyl-2-(3-(6-oxo-2-azaspiro[3.3]heptan-2-yl)isoxazol-5-yl)butanoate (230 mg, 420 umol, 56% purity, Intermediate FH) in THF (10 mL) was added (R)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (150 mg, 323 umol, Intermediate B), 4 Å molecular sieves (0.5 g), KOAc (63.4 mg, 645 umol) and AcOH (38.8 mg, 645 umol). The mixture was stirred at 0° C. for 1 hour, then NaBH(OAc)₃ (171 mg, 807 umol) was added and the mixture was stirred at 20° C. for 1 hour. On completion, the mixture was quenched with MeOH (0.5 mL), then filtered and the filtrate was concentrated under reduced pressure to give a crude product. The crude product was purified by reversed-phase HPLC (0.1% FA condition) to give the title compound (82 mg, 31% yield) as a light yellow solid. LC-MS (ESI⁺) m/z 732.3 (M+H)⁺.

Step 2-2-(3-(6-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptan-2-yl)isoxazol-5-yl)-3-methylbutanoic acid. To a solution of ethyl 2-[3-[6-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-2-azaspiro[3.3]heptan-2-yl]isoxazol-5-yl]-3-methyl-butanoate (82 mg, 112 umol) in THF (8 mL) and H₂O (8 mL) was added NaOH (22.4 mg, 560 umol). The mixture was stirred at 40° C. for 24 hours. On completion, the mixture was concentrated under reduced pressure, then dried by lyophilization to give the title compound (80 mg) as a yellow solid. LC-MS (ESI⁺) m/z 704.6 (M+H)⁺.

### (2S,4R)-4-hydroxy-1-(3-methyl-2-(3-(4-oxobutoxy)isoxazol-5-yl)butanoyl)-N-(4-(4-methylthiazol-5-yl)benzyl)pyrrolidine-2-carboxamide (Intermediate AD)

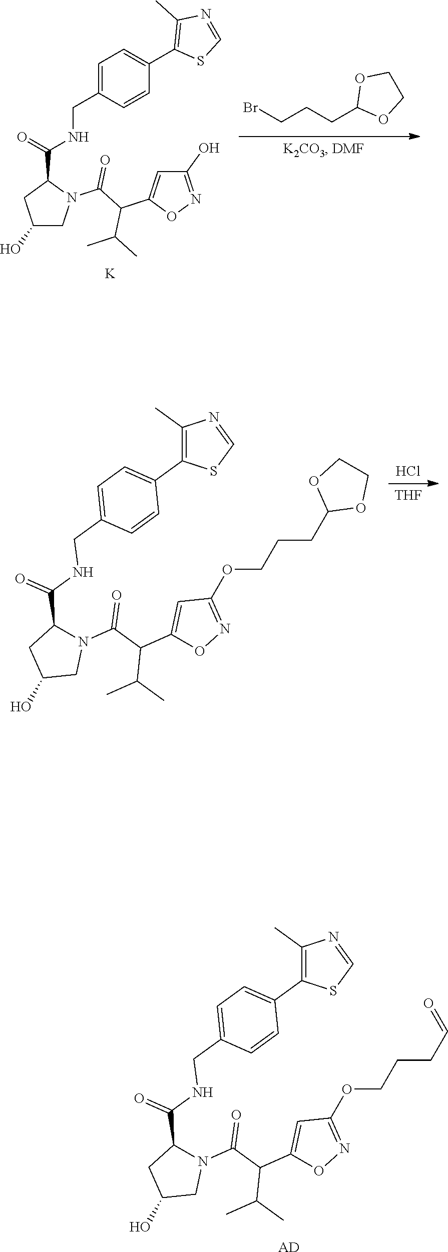

Step 1-(2S,4R)-1-(2-(3-(3-(1,3-dioxolan-2-yl)propoxy)isoxazol-5-yl)-3-methylbutanoyl)-4-hydroxy-N-(4-(4-methylthiazol-5-yl)benzyl)pyrrolidine-2-carboxamide. To a solution of (2S,4R)-4-hydroxy-1-[2-(3-hydroxyisoxazol-5-yl)-3-methyl-butanoyl]-N-[[4-(4-methylthiazol-5-yl)phenyl]methyl]pyrrolidine-2-carboxamide (100 mg, 206 umol, Intermediate K) in DMF (2 mL) was added K₂CO₃ (85.5 mg, 619 umol) and 2-(3-bromopropyl)-1,3-dioxolane (40.5 mg, 206 umol, CAS #62563-07-9). The mixture was stirred at 80° C. for 12 hours. On completion, the reaction mixture was filtered to give a filtrate and concentrated under reduced pressure to give the title compound (120 mg) as a yellow oil. LC-MS (ESI⁺) m/z 599.5 (M+H)⁺.

Step 2-(2S,4R)-4-hydroxy-1-(3-methyl-2-(3-(4-oxobutoxy)isoxazol-5-yl)butanoyl)-N-(4-(4-methylthiazol-5-yl)benzyl)pyrrolidine-2-carboxamide. To a solution of (2S,4R)-1-[2-[3-[3-(1,3-dioxolan-2-yl)propoxy]isoxazol-5-yl]-3-methyl-butanoyl]-4-hydroxy-N-[[4-(4-methylthiazol-5-yl)phenyl]methyl]pyrrolidine-2-carboxamide (120 mg, 200 umol) in THF (2 mL) was added HCl (1 M, 200 uL). The mixture was stirred at 50° C. for 12 hours. On completion, the reaction mixture was concentrated under reduced pressure to give a residue. The residue was purified by prep-HPLC (FA condition) to give the title compound (80 mg, 71% yield) as a white solid. LC-MS (ESI⁺) m/z 555.0 (M+H)⁺.

### 2-(3-(6-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptan-2-yl)isoxazol-5-yl)-3-methylbutanoic acid (Intermediate AE)

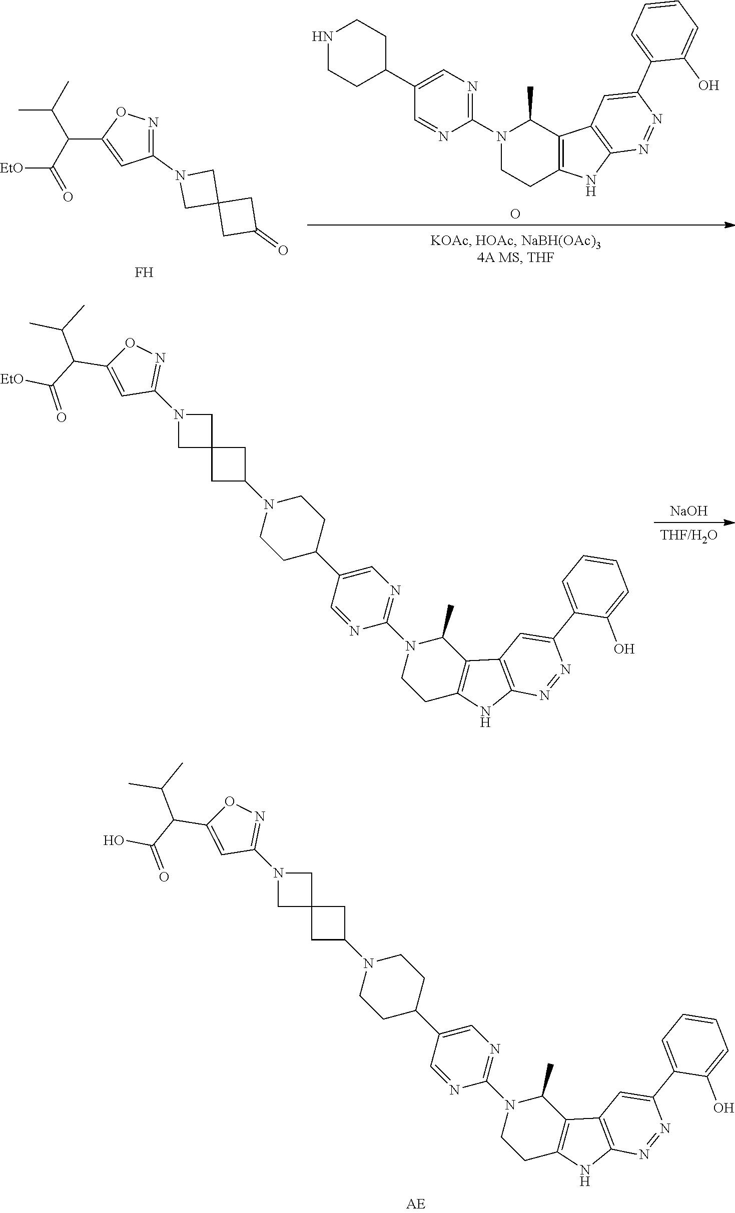

Step 1-Ethyl 2-(3-(6-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptan-2-yl)isoxazol-5-yl)-3-methylbutanoate. To a solution of ethyl 3-methyl-2-(3-(6-oxo-2-azaspiro[3.3]heptan-2-yl)isoxazol-5-yl)butanoate (230 mg, 420 umol, 56% purity, Intermediate FH) in THF (10 mL) was added (S)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (150 mg, 323 umol, Intermediate O), 4 Å molecular sieves (0.5 g), KOAc (63.4 mg, 645 umol) and AcOH (38.8 mg, 645 umol). The mixture was stirred at 0° C. for 1 hour, then NaBH(OAc)₃ (171 mg, 807 umol) was added and the mixture was stirred at 20° C. for 1 hour. On completion, the mixture was quenched with MeOH (0.5 mL), then filtered and the filtrate was concentrated under reduced pressure to give a crude product. The crude product was purified by reversed-phase HPLC (0.1% FA condition) to give the title compound (110 mg, 41% yield) as a light yellow solid. LC-MS (ESI⁺) m/z 732.4 (M+H)⁺.

Step 2-2-(3-(6-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptan-2-yl)isoxazol-5-yl)-3-methylbutanoic acid. To a solution of ethyl 2-[3-[6-[4-[2-[(3S)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-2-azaspiro[3.3]heptan-2-yl]isoxazol-5-yl]-3-methyl-butanoate (110 mg, 150 umol) in THF (8 mL) and H₂O (8 mL) was added NaOH (22.4 mg, 560 umol). The mixture was stirred at 40° C. for 24 hours. On completion, the mixture was concentrated under reduced pressure, then dried by lyophilization to give the title compound (130 mg) as a yellow solid. LC-MS (ESI⁺) m/z 704.2 (M+H)⁺.

### Ethyl 2-(3-(3-bromopropoxy)isoxazol-5-yl)-3-methylbutanoate (Intermediate AF)

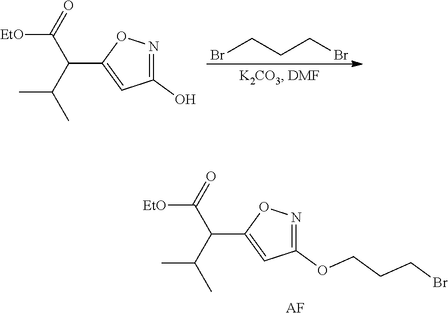 [SMILES: CCOC(=O)C(c1cc(O)no1)C(C)C ; BrCCCBr ; CCOC(=O)C(c1cc(OCCCBr)no1)C(C)C ; "AF"]

To a solution of ethyl 2-(3-hydroxyisoxazol-5-yl)-3-methyl-butanoate (3 g, 14.1 mmol, Intermediate FC) in DMF (20 mL) was added K₂CO₃ (5.83 g, 42.2 mmol) and 1,3-dibromopropane (8.52 g, 42.2 mmol, CAS #109-64-8) at 25° C. The mixture was then stirred at 60° C. for 3 hours. On completion, the reaction mixture was quenched with water (30 mL) and extracted by ethyl acetate (3×50 mL). The extracts were washed by brine (20 mL) and dried over anhydrous sodium sulfate, filtered and concentrated in vacuo to get the crude residue. The residue was purified by column chromatography (SiO₂, Petroleum ether/Ethyl acetate=0/1 to 10/1) to give the title compound (2.5 g, 42% yield) as yellow oil. LC-MS (ESI⁺) m/z 355.4 (M+H)⁺.

### 2-(3-(3-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoic acid (Intermediate AG)

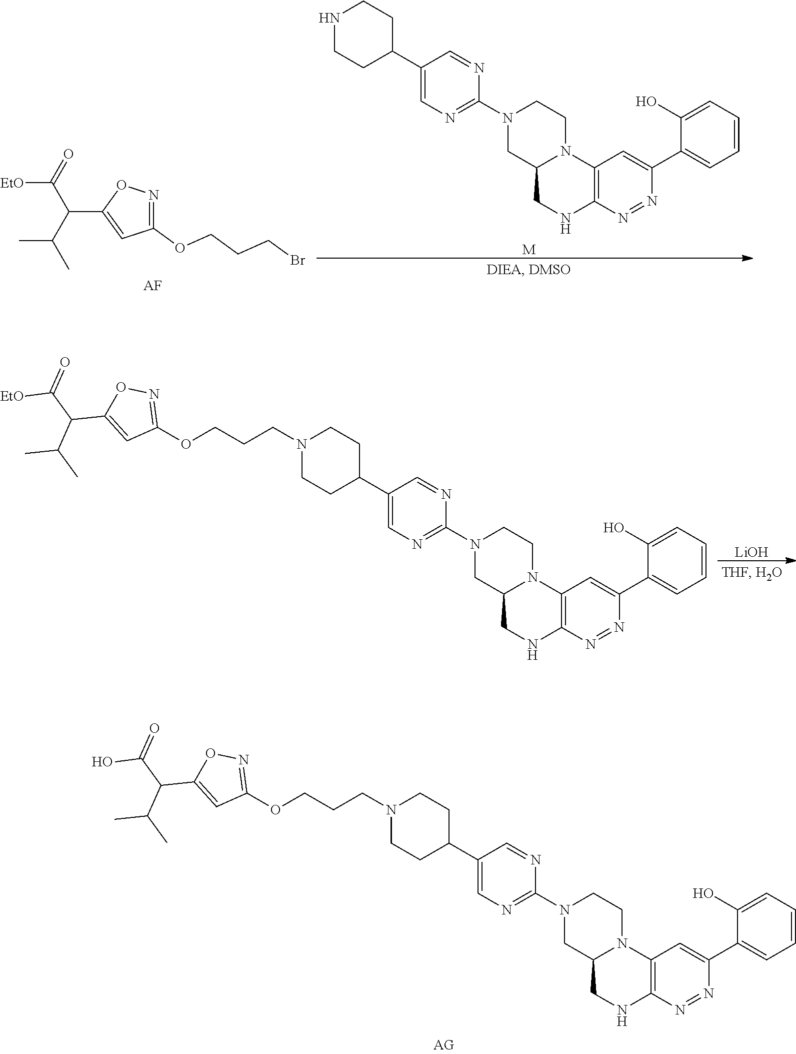

Step 1-Ethyl 2-(3-(3-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoate. To a solution of ethyl 2-(3-(3-bromopropoxy)isoxazol-5-yl)-3-methylbutanoate (100 mg, 299 umol, Intermediate AF) in DMSO (1 mL) was added DIEA (116 mg, 898 umol), KI (74.5 mg, 449 umol) and 2-[(10S)-12-[5-(4-piperidyl)pyrimidin-2-yl]-1,5,6,8,12-pentazatricyclo[8.4.0.02,7]tetradeca-2(7),3,5-trien-4-yl]phenol (133 mg, 299 umol, Intermediate M) at 25° C. The mixture was then stirred at 50° C. for 5 hours. On completion, the reaction mixture was quenched with water (5 mL) and extracted by ethyl acetate (3×10 mL). The extracts were washed with brine (10 mL) and dried over anhydrous sodium sulfate, filtered and concentrated in vacuo to get the crude residue. The crude product was purified by reversed-phase HPLC (0.1% FA condition) to give the title compound (110 mg, 53% yield) as white solid. LC-MS (ESI⁺) m/z 698.4. (M+H)⁺.

Step 2-2-(3-(3-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoic acid. To a solution of ethyl 2-(3-(3-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoate (110 mg, 157 umol) in THF (2.5 mL) was added LiOH H₂O (33.1 mg, 788 umol) at 25° C. Then the mixture was stirred at 25° C. for 2 hours. On completion, to the reaction mixture was added HCl (1N) until pH=4. The mixture was then concentrated in vacuo to give the title compound (140 mg) as yellow solid.

### Ethyl 2-[3-(3-bromopropoxy)isoxazol-5-yl]-3-methyl-butanoate (Intermediate AH)

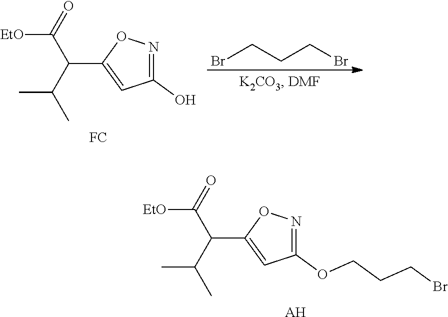

To a solution of ethyl 2-(3-hydroxyisoxazol-5-yl)-3-methyl-butanoate (3 g, 14.1 mmol, Intermediate FC) in DMF (20 mL) was added K₂CO₃ (5.83 g, 42.2 mmol) and 1,3-dibromopropane (8.52 g, 42.2 mmol, CAS #109-64-8) at 25° C. The mixture was stirred at 60° C. for 3 hours. On completion, the reaction mixture was quenched with water (30 mL) and extracted by ethyl acetate (3×50 mL). The extracts were washed with brine (20 mL) and dried over anhydrous sodium sulfate, filtered and concentrated in vacuo to get the crude residue. The residue was purified by column chromatography (SiO₂, Petroleum ether/Ethyl acetate=0/1 to 10/1) to give the title compound (2.5 g, 42% yield) as yellow oil. LC-MS (ESI⁺) m z 355.4. (M+H)⁺.

### 2-(3-(3-(4-(2-((R)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoic acid (Intermediate AI)

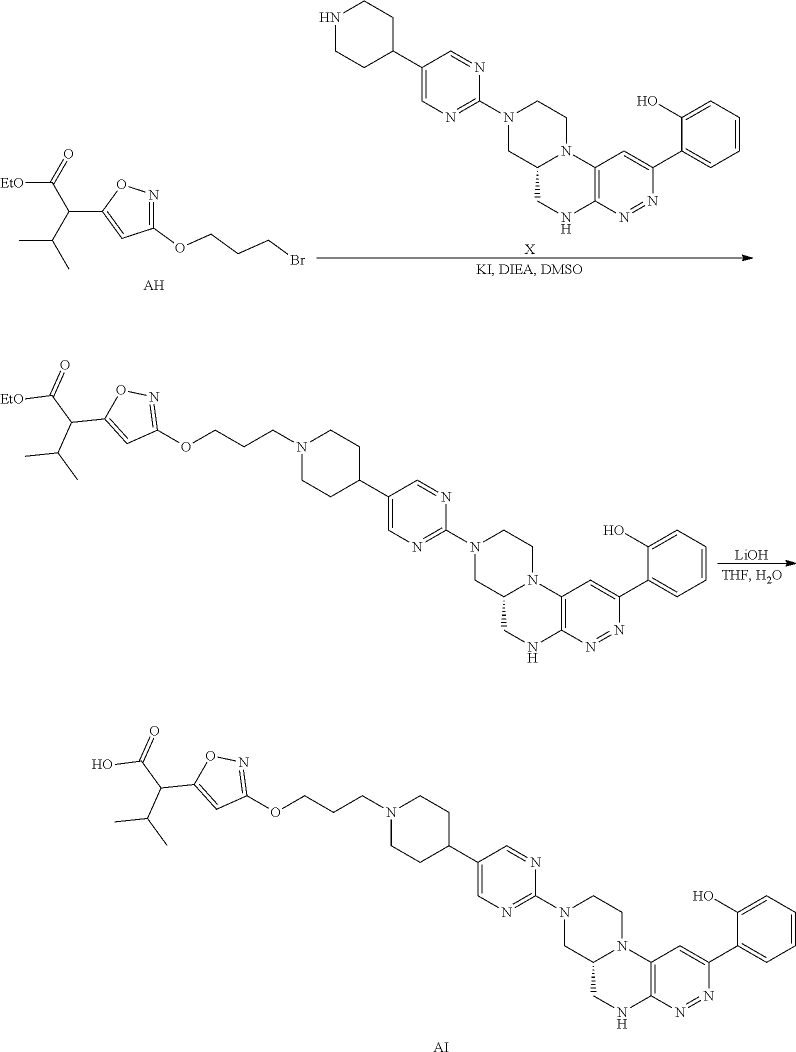

Step 1-Ethyl 2-(3-(3-(4-(2-((R)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoate. To a solution of ethyl 2-[3-(3-bromopropoxy)isoxazol-5-yl]-3-methyl-butanoate (50.46 mg, 151 umol, Intermediate AH) in DMSO (1 mL) was added DIEA (58.5 mg, 453 umol), KI (37.6 mg, 226 umol) and 2-[(1OR)-12-[5-(4-piperidyl)pyrimidin-2-yl]-1,5,6,8,12-pentazatricyclo[8.4.0.02,7]tetradeca-2(7),3,5-trien-4-yl]phenol (67.1 mg, 151 umol, Intermediate X) at 25° C. The mixture was stirred at 50° C. for 5 hours. On completion, the reaction mixture was quenched with water (5 mL) and extracted with ethyl acetate (3×10 mL). The extracts were washed with brine (10 mL) and dried over anhydrous sodium sulfate, filtered and concentrated in vacuo to get the crude residue. The crude product was purified by reversed-phase HPLC (0.1% FA condition) to give the title compound (110 mg, 53% yield) as white solid. LC-MS (ESI⁺) m/z 698.5 (M+H)⁺.

Step 2 2-(3-(3-(4-(2-((R)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoic acid. To a solution of ethyl 2-[3-[3-[4-[2-[(10R)-4-(2-hydroxyphenyl)-1,5,6,8,12-pentazatricyclo[8.4.0.02,7]tetradeca-2(7),3,5-trien-12-yl]pyrimidin-5-yl]-1-piperidyl]propoxy]isoxazol-5-yl]-3-methyl-butanoate (55 mg, 78.8 umol) in THF (1 mL) and H₂O (1 mL) was added LiOH H₂O (16.5 mg, 394 umol) at 25° C. Then the mixture was stirred at 25° C. for 2 hours. On completion, to the reaction mixture was added HCl (1N) until the pH=4, Then the mixture was concentrated in vacuo to give the title compound (80 mg) as white solid.

### (S)-2-(6-(5-(1-(7-Azaspiro[3.5]nonan-2-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate AJ)

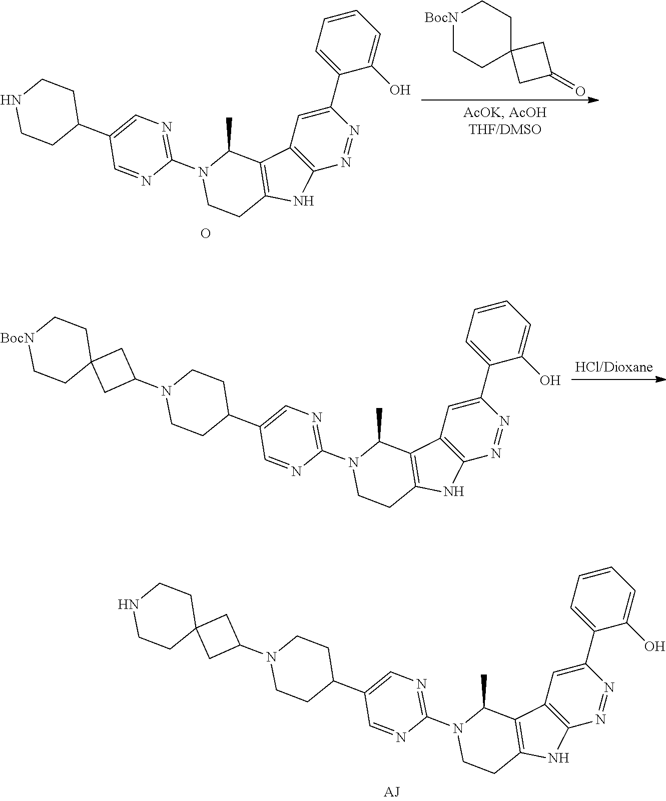

Step 1-(S)-tert-butyl 2-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonane-7-carboxylate. To a solution of 2-[(3S)-3-methyl-4-[5-(4-piperidyl)pyrimidin-2-yl]-4,8,10,11-tetrazatricyclo[7.4.0.0^{2,7}]trideca-1(9),2(7),10,12-tetraen-12-yl]phenol (150 mg, 339 umol, Intermediate O) in DMSO (0.8 mL), THF (4 mL) was added AcOK (100 mg, 1.02 mmol) and stirred at 50° C. for 10 minutes. Then tert-butyl 2-oxo-7-azaspiro[3.5]nonane-7-carboxylate (114 mg, 476 umol, CAS #203661-69-2) and AcOH (61.2 mg, 1.02 mmol, 58.29 uL) was added and the mixture was stirred for another 3 hours. At last, NaBH(OAc)₃ (180 mg, 849 umol) was added at 0° C. and the mixture was stirred at 25° C. for 12 hours. On completion, the reaction mixture was diluted with water 5 mL and extracted with EA 15 mL (5 mL×3). The combined organic layers were washed with aqueous NaCl 15 mL (5 mL×3), dried over Na₂SO₄, filtered and concentrated under reduced pressure to give a residue. The residue was purified by prep-HPLC (FA condition) to give the title compound (142 mg, 58% yield) as a yellow solid. LC-MS (ESI⁺) m/z 665.6 (M+H)⁺.

Step 2-(S)-2-(6-(5-(1-(7-azaspiro[3.5]nonan-2-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 2-[4-[2-[(3S)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-7-azaspiro[3.5]nonane-7-carboxylate (90 mg, 127 umol, FA) in DCM (5 mL) was added HCl/dioxane (4 M, 253 uL). The mixture was stirred at 25° C. for 1 hour. On completion, the mixture was concentrated under reduced pressure to give the residue (130 mg) as a yellow solid. LC-MS (ESI⁺) m/z 565.5 (M+H)⁺.

### Tert-butyl 4-(2-chloropyrimidin-4-yl)piperidine-1-carboxylate (Intermediate AK)

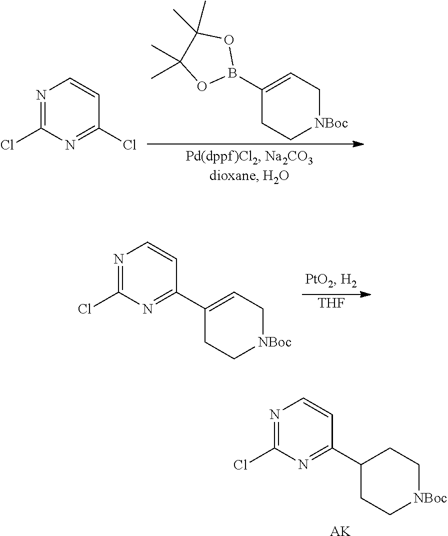 [SMILES: Clc1ccnc(Cl)n1 ; CC(C)(C)OC(=O)N1CC=C(B2OC(C)(C)C(C)(C)O2)CC1 ; CC(C)(C)OC(=O)N1CC=C(c2ccnc(Cl)n2)CC1 ; CC(C)(C)OC(=O)N1CCC(c2ccnc(Cl)n2)CC1 ; "AK" ; "THF"]

Step 1-Tert-butyl 4-(2-chloropyrimidin-4-yl)-5,6-dihydropyridine-1(2H)-carboxylate. To a solution of 2,4-dichloropyrimidine (36 g, 241 mmol) in dioxane (500 mL) and H₂O (50 mL) was added tertbutyl-4-(4,4,5,5-tetramethyl-1,3,2-dioxaborolan-2-yl)-3,6-dihydro-2H-pyridine-1-carboxylate (89.6 g, 289 mmol), Pd(dppf)Cl₂ (17.7 g, 24.2 mmol), and Na₂CO₃ (76.8 g, 725 mmol). Then the mixture was stirred at 80° C. for 12 hours. On completion, the reaction mixture was diluted with H₂O (600 mL) and extracted with EA (300 mL×3). The combined organic layers were washed with brine (200 mL×2), dried over Na₂SO₄, filtered and concentrated under reduced pressure to give a residue. The residue was purified by column chromatography (SiO₂, Petroleum ether/Ethyl acetate=5/1 to 2/1) to give the title compound (62 g, 87% yield) as white solid. LC-MS (ESI⁺) m/z 296.1 (M+H)⁺.

Step 2-tert-butyl 4-(2-chloropyrimidin-4-yl)piperidine-1-carboxylate. To a solution of tert-butyl 4-(2-chloropyrimidin-4-yl)-3,6-dihydro-2H-pyridine-1-carboxylate (62 g, 209) in THF (700 mL) was added PtO₂ (23 g, 102 mmol). Then the mixture was stirred at 30° C. for 24 hours under H₂ atmosphere. On completion, the reaction mixture was filtered and concentrated under reduced pressure to give a residue. The residue was purified by column chromatography (SiO₂, Petroleum ether/Ethyl acetate=3/1 to 1/1) to give the title compound (42 g, 66% yield) as a white solid. LC-MS (ESI⁺) m/z 298.1 (M+H)⁺.

### (2S,4R)-4-Hydroxy-1-(3-methyl-2-(3-(2-oxoethoxy)isoxazol-5-yl)butanoyl)-N-(4-(4-methylthiazol-5-yl)benzyl)pyrrolidine-2-carboxamide (Intermediate AL)

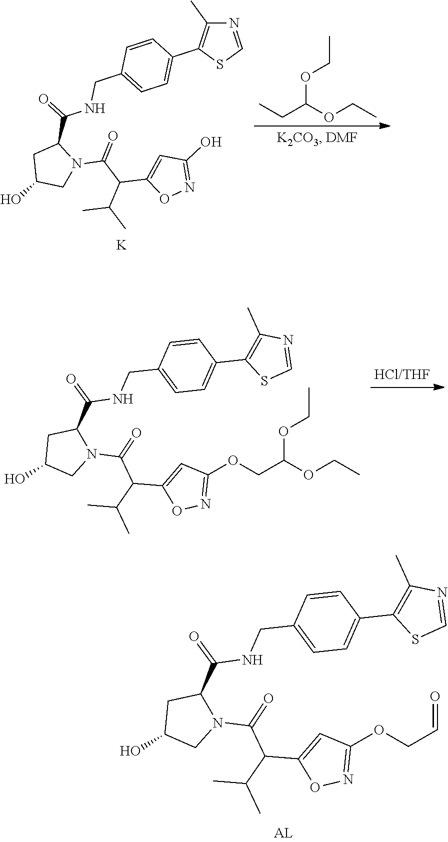

Step 1-(2S,4R)-1-(2-(3-(2,2-diethoxyethoxy)isoxazol-5-yl)-3-methylbutanoyl)-4-hydroxy-N-(4-(4-methylthiazol-5-yl)benzyl)pyrrolidine-2-carboxamide. To a solution of (2S,4R)-4-hydroxy-1-[2-(3-hydroxyisoxazol-5-yl)-3-methyl-butanoyl]-N-[[4-(4-methylthiazol-5-yl)phenyl]methyl]pyrrolidine-2-carboxamide (150 mg, 309 umol, Intermediate K) in DMF (2 mL) was added K₂CO₃ (128 mg, 928 umol) and 2-bromo-1,1-diethoxy-ethane (61.0 mg, 309 umol). The mixture was stirred at 80° C. for 12 hours. On completion, the reaction mixture was filtered to give a filtrate. The filtrate was concentrated under reduced pressure to give the title compound (160 mg) as a yellow solid. LC-MS (ESI⁺) m/z 601.3 (M+H)⁺.

Step 2-(2S,4R)-4-hydroxy-1-(3-methyl-2-(3-(2-oxoethoxy)isoxazol-5-yl)butanoyl)-N-(4-(4-methylthiazol-5-yl)benzyl)pyrrolidine-2-carboxamide. To a solution of (2S,4R)-1-[2-[3-(2,2-diethoxyethoxy)isoxazol-5-yl]-3-methyl-butanoyl]-4-hydroxy-N-[[4-(4-methylthiazol-5-yl)phenyl]methyl]pyrrolidine-2-carboxamide (160 mg, 266 umol) in THF (2 mL) was added HCl (1 M, 500 uL). The mixture was stirred at 50° C. for 12 hours. On completion, the reaction mixture was filtered to give the title compound (160 mg) was a yellow solid. LC-MS (ESI⁺) m/z 527.1 (M+H)⁺.

### (2S,4R)-4-Hydroxy-1-(3-methyl-2-(3-(prop-2-yn-1-yloxy)isoxazol-5-yl)butanoyl)-N-(4-(4-methylthiazol-5-yl)-2-((4-oxocyclohexyl)oxy)benzyl)pyrrolidine-2-carboxamide (Intermediate AM)

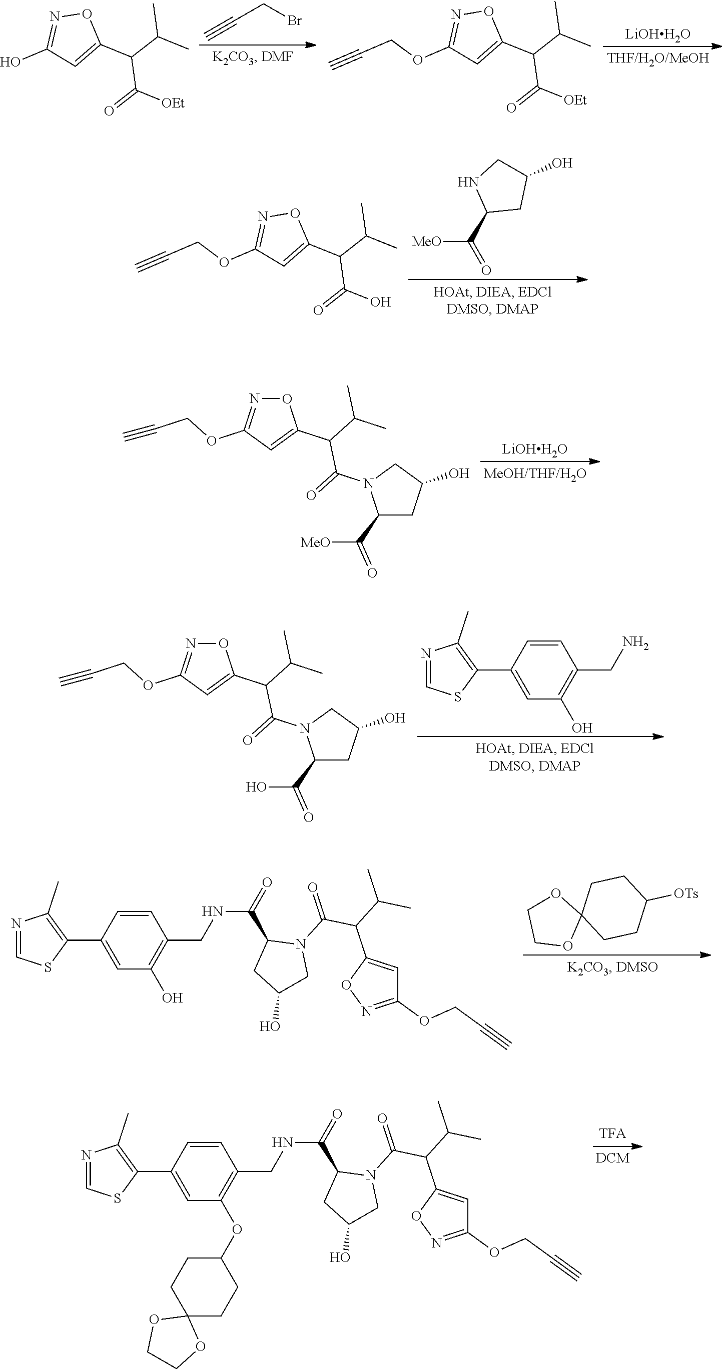

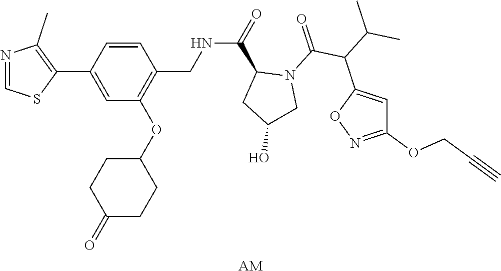

Step 1-Ethyl 3-methyl-2-(3-(prop-2-yn-1-yloxy)isoxazol-5-yl)butanoate. To a solution of ethyl 2-(3-hydroxyisoxazol-5-yl)-3-methyl-butanoate (2.5 g, 9.38 mmol, 80% purity, Intermediate FC) in DMF (20 mL) was added K₂CO₃ (2.59 g, 18.8 mmol) and 3-bromoprop-1-yne (1.53 g, 10.3 mmol, 80% solution). The mixture was stirred at 25° C. for 4 hours. On completion, the reaction mixture was diluted with water 20 mL and extracted with CH₂Cl₂ 60 mL (20 mL×3). The combined organic layers were washed with aqueous NaCl 60 mL (20 mL×3), dried over Na₂SO₄, filtered and concentrated under reduced pressure to give a residue. The residue was purified by column chromatography (SiO₂, Petroleum ether/Ethyl acetate=15/1 to 10/1) to give the title compound (1.28 g, 48% yield) as a white oil. LC-MS (ESI⁺) m/z 252.1(M+H)⁺.

Step 2-3-Methyl-2-(3-(prop-2-yn-1-yloxy)isoxazol-5-yl)butanoic acid. To a mixture of tert-butyl tert-butyl 4-[[chloro-(3-chloro-2-fluoro-phenyl)-oxo-dispiro[BLAH]carbonyl]amino]piperidine-1-carboxylate (50.0 mg, 77.4 umol) in DCM (0.5 mL) was added HCl/dioxane (4 M, 0.1 mL) in one portion at 25° C. under N₂. The mixture was stirred at 25° C. for 30 minutes. On completion, the reaction mixture was concentrated in vacuo to give the title compound (50.0 mg) as a white solid. LC-MS (ESI⁺) m/z 224.0 (M+H)⁺.

Step 3-(2S,4R)-methyl 4-hydroxy-1-(3-methyl-2-(3-(prop-2-yn-1-yloxy)isoxazol-5-yl)butanoyl)pyrrolidine-2-carboxylate. To a solution of 3-methyl-2-(3-prop-2-ynoxyisoxazol-5-yl)butanoic acid (740 mg, 2.85 mmol) in DMSO (6 mL) was added HOAt (582 mg, 4.27 mmol, 598 uL), DMAP (69.6 mg, 570 umol), EDCI (819 mg, 4.27 mmol) DIEA (1.47 g, 11.4 mmol, 1.99 mL) and methyl (2S,4R)-4-hydroxypyrrolidine-2-carboxylate (932 mg, 5.13 mmol, HCl salt). The mixture was stirred at 40° C. for 2 hours. On completion, the reaction mixture was diluted with water 10 mL and extracted with EA 15 mL (5 mL×3). The combined organic layers were washed with aqueous NaCl 15 mL (5 mL×3), dried over Na₂SO₄, filtered and concentrated under reduced pressure to give a residue. The residue was purified by prep-HPLC (HCl condition) to give the title compound (820 mg, 74% yield) as a yellow solid. LC-MS (ESI⁺) m/z 351.1 (M+H)⁺.

Step 4-(2S,4R)-4-hydroxy-1-(3-methyl-2-(3-(prop-2-yn-1-yloxy)isoxazol-5-yl)butanoyl)pyrrolidine-2-carboxylic acid. To a solution of methyl (2S,4R)-4-hydroxy-1-[3-methyl-2-(3-prop-2-ynoxyisoxazol-5-yl)butanoyl]pyrrolidine-2-carboxylate (800 mg, 2.28 mmol) in THF (4 mL), MeOH (4 mL) and H₂O (4 mL) was added LiOH·H₂O (383 mg, 9.13 mmol). The mixture was stirred at 25° C. for 2 hours. On completion, the pH value of the mixture was adjusted to 5 by adding 1M citric acid. Then the solution was concentrated under reduced pressure to give the residue (0.9 g) as a white solid. LC-MS (ESI⁺) m/z 337.1 (M+H)⁺.

Step 5-(2S,4R)-4-hydroxy-N-(2-hydroxy-4-(4-methylthiazol-5-yl)benzyl)-1-(3-methyl-2-(3-(prop-2-yn-1-yloxy)isoxazol-5-yl)butanoyl)pyrrolidine-2-carboxamide. To a solution of 2-(aminomethyl)-5-(4-methylthiazol-5-yl)phenol (149 mg, 676 umol, CAS #1448190-11-1) in DMSO (3 mL) was added HOAt (91.1 mg, 669 umol, 93.6 uL), DIEA (403 mg, 3.12 mmol, 544 uL), EDCI (128 mg, 669 umol), DMAP (10.9 mg, 89.2 umol) and (2S,4R)-4-hydroxy-1-[3-methyl-2-(3-prop-2-ynoxyisoxazol-5-yl)butanoyl]pyrrolidine-2-carboxylic acid (150 mg, 446 umol). The mixture was stirred at 40° C. for 2 hours. On completion, the reaction mixture was diluted with water 3 mL and extracted with EA (3 mL×3). The combined organic layers were washed with aqueous NaCl 15 mL (5 mL×3), dried over Na₂SO₄, filtered and concentrated under reduced pressure to give a residue. The residue was purified by prep-HPLC (FA condition) to give the title compound (85 mg, 32% yield) as a white solid. LC-MS (ESI⁺) m/z 539.3 (M+H)⁺.

Step 6-(2S,4R)—N-(2-(1,4-dioxaspiro[4.5]decan-8-yloxy)-4-(4-methylthiazol-5-yl)benzyl)-4-hydroxy-1-(3-methyl-2-(3-(prop-2-yn-1-yloxy)isoxazol-5-yl)butanoyl)pyrrolidine-2-carboxamide. To a solution of (2S,4R)-4-hydroxy-N-[[2-hydroxy-4-(4-methylthiazol-5-yl)phenyl]methyl]-1-[3-methyl-2-(3-prop-2-ynoxyisoxazol-5-yl)butanoyl]pyrrolidine-2-carboxamide (75 mg, 139 umol) in DMSO (3 mL) was added K₂CO₃ (38.5 mg, 278 umol) and 1,4-dioxaspiro[4.5]decan-8-yl 4-methylbenzenesulfonate (109 mg, 348 umol, CAS #23511-05-9). The mixture was stirred at 80° C. for 12 hours. On completion, the reaction mixture was diluted with water (5 mL) and extracted with EA (3 mL×3). The combined organic layers were washed with aqueous NaCl (3 mL×3), dried over Na₂SO₄, filtered and concentrated under reduced pressure to give a residue. The residue was purified by prep-HPLC (FA condition) to give the title compound (82 mg, 80% yield) as a white solid. LC-MS (ESI⁺) m/z 679.4 (M+H)⁺.

Step 7-(2S,4R)-4-hydroxy-1-(3-methyl-2-(3-(prop-2-yn-1-yloxy)isoxazol-5-yl)butanoyl)-N-(4-(4-methylthiazol-5-yl)-2-((4-oxocyclohexyl)oxy)benzyl)pyrrolidine-2-carboxamide. To a solution of (2S,4R)—N-[[2-(1,4-dioxaspiro[4.5]decan-8-yloxy)-4-(4-methylthiazol-5-yl)phenyl]methyl]-4-hydroxy-1-[3-methyl-2-(3-prop-2-ynoxyisoxazol-5-yl)butanoyl]pyrrolidine-2-carboxamide (70 mg, 103 umol) in CH₂Cl₂ (12 mL) was added TFA (5.39 g, 47.3 mmol, 3.50 mL). The mixture was stirred at 25° C. for 2 hours. On completion, the mixture was concentrated under reduced pressure to give the title compound (150 mg, TFA salt) as a white solid. LC-MS (ESI⁺) m/z 635.4 (M+H)⁺.

### 2-(3-((4-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)-methoxy)isoxazol-5-yl)-3-methylbutanoic acid (Intermediate AN)

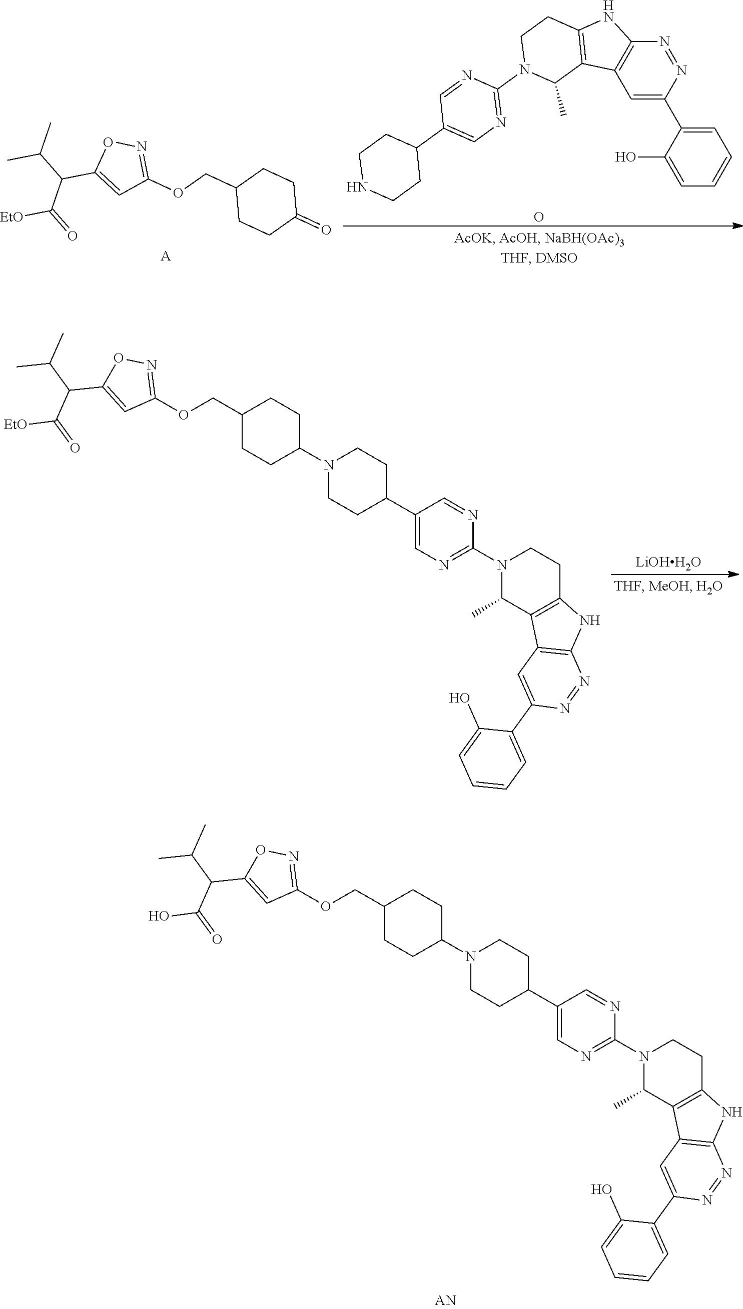

Step 1-Ethyl 2-(3-((4-(4-(2-((S)-3-(2-hydroxyphenyl)-5-methyl-7,8-dihydro-5H-pyrido[3,4′:4,5]pyrrolo[2,3-c]pyridazin-6(9H)-yl)pyrimidin-5-yl)piperidin-1-yl)cyclohexyl)-methoxy)isoxazol-5-yl)-3-methylbutanoate. To a solution of 2-[(3S)-3-methyl-4-[5-(4-piperidyl)pyrimidin-2-yl]-4,8,10,11-tetrazatricyclo[7.4.0.0^{2,7}]trideca-1(9),2(7),10,12-tetraen-12-yl]phenol (300 mg, 679 umol, Intermediate O) in DMSO (0.5 mL) and THF (5 mL) was added AcOK (100 mg, 1.02 mmol) and the mixture was stirred for 30 minutes. Then ethyl 3-methyl-2-[3-[(4-oxocyclohexyl)methoxy]isoxazol-5-yl]butanoate (329 mg, 1.02 mmol, Intermediate A) and AcOH (61.2 mg, 1.02 mmol) was added and the mixture was stirred 12 hours. Then NaBH(OAc)₃ (432 mg, 2.04 mmol) was added at 0° C. and the mixture was stirred at 50° C. for 2.5 hours. On completion, the reaction mixture filtered and concentrated under reduced pressure to give a residue. The residue was purified by prep-HPLC (neutral condition column: Waters Xbridge 150*25 mm*5 um; mobile phase: [water(10 mM NH₄HCO₃)-ACN]; B %: 67%-87%, 10 min) to give the title compound (190 mg, 74% yield) as a yellow solid. LC-MS (ESI⁺) m/z 749.4 (M+H)⁺.

Step 2-2-(3-((4-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)-methoxy)isoxazol-5-yl)-3-methylbutanoic acid. To a solution of ethyl 2-(3-((4-(4-(2-((S)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)-methoxy)isoxazol-5-yl)-3-methylbutanoate (190 mg, 251 umol) in THF (0.5 mL), MeOH (0.5 mL) and H₂O (0.5 mL) was added LiOH·H₂O (6.72 mg, 160 umol). The mixture was stirred at 25° C. for 2 hours. On completion, the reaction mixture was adjusted the pH to 7 and concentrated under reduced pressure to give the title compound (190 mg) as a yellow solid. LC-MS (ESI⁺) m/z 721.6 (M+H)⁺.

### (R)-2-(6-(5-(1-(2-azaspiro[3.5]nonan-7-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate AO)

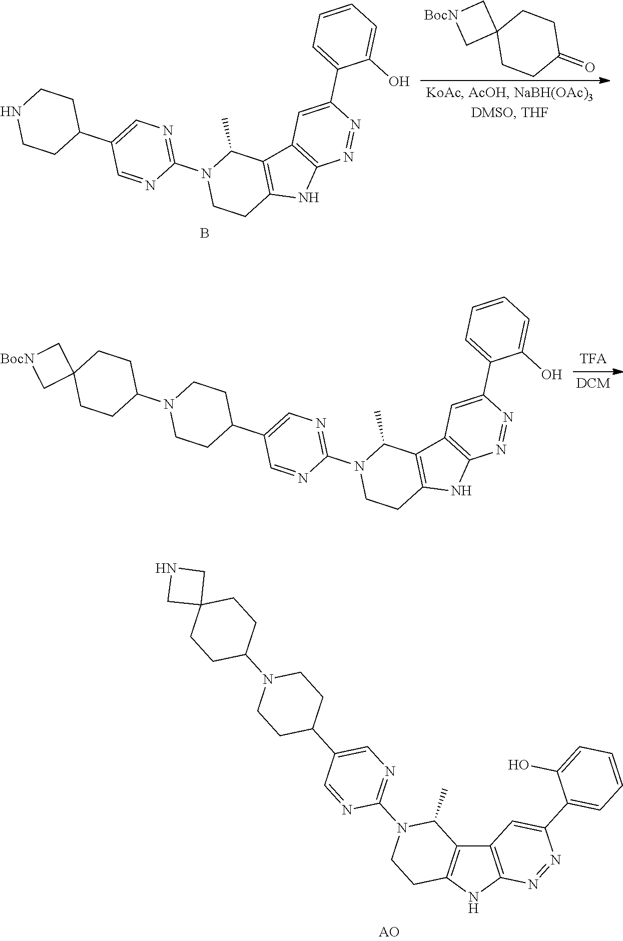

Step 1-(R)-tert-butyl 7-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.5]nonane-2-carboxylate. To a solution of 2-[(3R)-3-methyl-4-[5-(4-piperidyl)pyrimidin-2-yl]-4,8,10,11-tetrazatricyclo[7.4.0.0^{2,7}]trideca-1(9),2(7),10,12-tetraen-12-yl]phenol (0.2 g, 453 umol, Intermediate B) in THF (2 mL) and DMSO (1 mL) was added KOAc (133 mg, 1.36 mmol) and the mixture was stirred 30 minutes. Then add AcOH (109 mg, 1.81 mmol, 103.63 uL) and tert-butyl 7-oxo-2-azaspiro[3.5]nonane-2-carboxylate (217 mg, 906 umol, CAS #1363381-22-9) were added and the mixture was stirred for 30 minutes. Next, NaBH(OAc)₃ (288 mg, 1.36 mmol) was added and the mixture was stirred at 25° C. for 13 hours. On completion, the mixture was concentrated under reduce pressure to remove the THF. The crude product was purification by reversed-phase Flash(0.1% FA condition) to give the title compound (300 mg, 95% yield) as a white solid. LC-MS (ESI⁺) m/z 665.3 (M+H)⁺.

Step 2-(R)-2-(6-(5-(1-(2-azaspiro[3.5]nonan-7-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 7-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-2-azaspiro[3.5]nonane-2-carboxylate (0.25 g, 376 umol) in DCM (3 mL) was added TFA (385 mg, 3.38 mmol, 250 uL). The mixture was stirred at 25° C. for 3 hours. On completion, the mixture was concentrated under reduce pressure to give the title compound: (250 mg) as a white solid. LC-MS (ESI⁺) m/z 565.3 (M+H)⁺.

### (R)-2-(6-(5-(1-(7-azaspiro[3.5]nonan-2-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate AP)

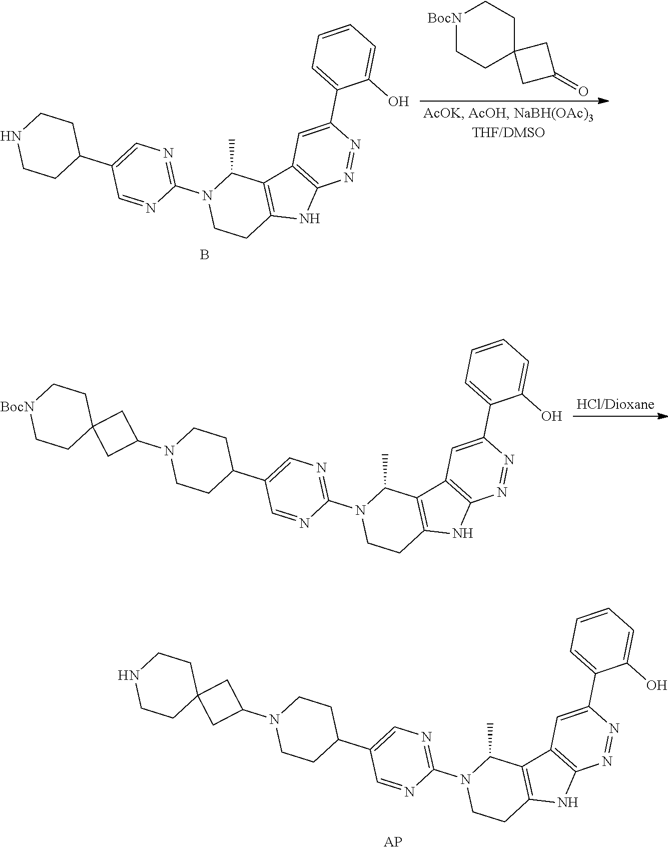

Step 1-(R)-tert-butyl 2-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-7-azaspiro[3.5]nonane-7-carboxylate. To a solution of 2-[(3R)-3-methyl-4-[5-(4-piperidyl)pyrimidin-2-yl]-4,8,10,11-tetrazatricyclo[7.4.0.0^{2,7}]trideca-1(9),2(7),10,12-tetraen-12-yl]phenol (230 mg, 521 umol, Intermediate B) in THF (4 mL) and DMSO (0.8 mL) was added AcOK (153 mg, 1.56 mmol) and the mixture was stirred at 40° C. for 10 minutes. Then tert-butyl 2-oxo-7-azaspiro[3.5]nonane-7-carboxylate (175 mg, 729 umol, CAS #203661-69-2) and AcOH (93.9 mg, 1.56 mmol, 89.38 uL) was added slowly and the reaction mixture was stirred for another 12 hours. At last, NaBH(OAc)₃ (276 mg, 1.30 mmol) was added at 0° C. and the reaction mixture was stirred at 25° C. for 3 hours. On completion, the mixture was concentrated under reduced pressure to give the residue. The residue was purified by prep-HPLC (FA condition) to give the title compound (120 mg, 27% yield) as a yellow solid. LC-MS (ESI⁺) m/z 665.6 (M+H)⁺.

Step 2-(R)-2-(6-(5-(1-(7-azaspiro[3.5]nonan-2-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 2-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-7-azaspiro[3.5]nonane-7-carboxylate (120 mg, 169 umol, FA) in DCM (10 mL) was added HCl/dioxane (4 M, 338 uL). The mixture was stirred at 25° C. for 2 hours. On completion, the mixture was concentrated under reduced pressure to give the residue (160 mg) as a white solid. LC-MS (ESI⁺) m/z 565.6 (M+H)⁺.

### 2-[(3S)-3-Methyl-4-[5-[1-(4-piperidyl)-4-piperidyl]pyrimidin-2-yl]-4,8,10,11-tetrazatricyclo[7.4.0.0^{2,7}]trideca-1(9),2(7),10,12-tetraen-12-yl]phenol (Intermediate AQ)

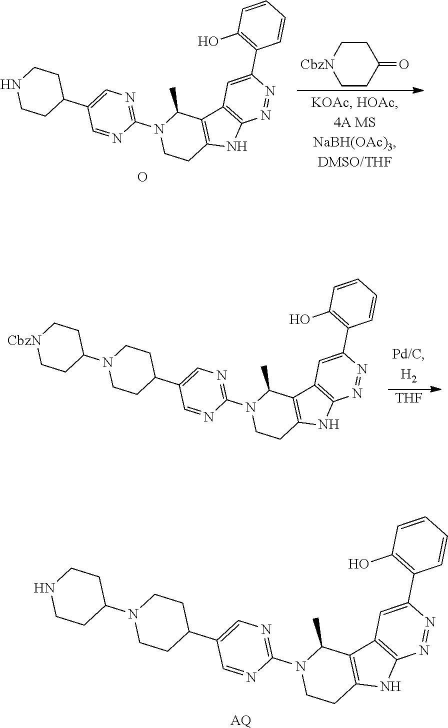

Step 1-(S)-benzyl 4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)-[1,4′-bipiperidine]-1′-carboxylate. To a solution of 2-[(3S)-3-methyl-4-[5-(4-piperidyl)pyrimidin-2-yl]-4,8,10,11-tetrazatricyclo[7.4.0.02,7]trideca-1(9),2(7),10,12-tetraen-12-yl]phenol (600 mg, 1.36 mmol, Intermediate O) in DMSO (1 mL) and THF (2 mL) was added KOAc (400 mg, 4.08 mmol), HOAc (244 mg, 4.08 mmol), 4 Å molecular sieves (200 mg) and benzyl 4-oxopiperidine-1-carboxylate (475 mg, 2.04 mmol, CAS #19099-93-5). The mixture was stirred at 0° C. for 1 hr, and then NaBH(OAc)₃ (864 mg 4.08 mmol) was added. The mixture was then stirred at 25° C. for 2 hours. On completion, the reaction mixture was quenched with H₂O (2 mL) at 0° C., then filtered and concentrated under reduced pressure to give a residue. The residue was purified by prep-HPLC (FA condition) to give the title compound (700 mg, 72% yield) as a yellow solid. LC-MS (ESI⁺) m/z 659.3 (M+H)⁺.

Step 2-(S)-2-(6-(5-([1,4′-bipiperidin]-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of benzyl 4-[4-[2-[(3S)-12-(2-hydroxyphenyl)-3-methyl-4,8,10,11-tetrazatricyclo[7.4.0.02,7]tridecal(9),2(7),10,12-tetraen-4-yl]pyrimidin-5-yl]-1-piperidyl]piperidine-1-carboxylate (600 mg, 910 umol) in THF (5 mL) was added Pd/C (10 wt %, 600 mg) under N₂. The suspension was degassed under vacuum and purged with H₂ several times. Then the mixture was stirred under H₂ (15 psi) at 20° C. for 12 hours. On completion, the reaction mixture was filtered and concentrated under reduced pressure to give the title compound (500 mg) as a yellow solid. LC-MS (ESI⁺) m/z 525.2 (M+H)⁺.

### (R)-2-(8-(4-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate AR)

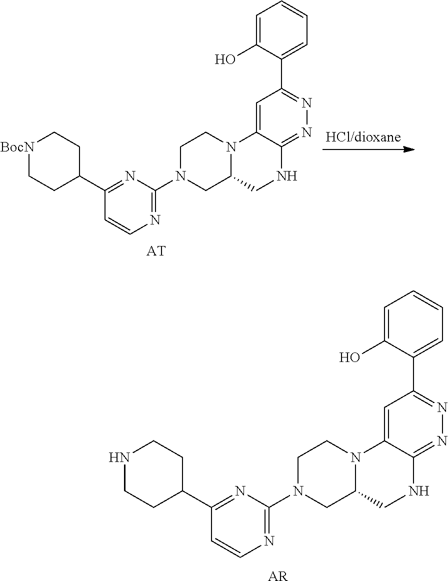

To a solution of tert-butyl 4-[2-[(1OR)-4-(2-hydroxyphenyl)-1,5,6,8,12-pentazatricyclo[8.4.0.0^{2,7}]tetradeca-2,4,6-trien-12-yl]pyrimidin-4-yl]piperidine-1-carboxylate (0.1 g, 184 umol, Intermediate AT) in DCM (1 mL) was added HCl/dioxane (4 M, 0.4 mL). The mixture was stirred at 25° C. for 12 hours. On completion, the mixture was concentrated under reduce pressure to give the title compound (100 mg) as a white solid. LC-MS (ESI⁺) m/z 445.5 (M+H)⁺.

### (R)-6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidin-1-yl)spiro[3.3]heptane-2-carboxylic acid (Intermediate AS)

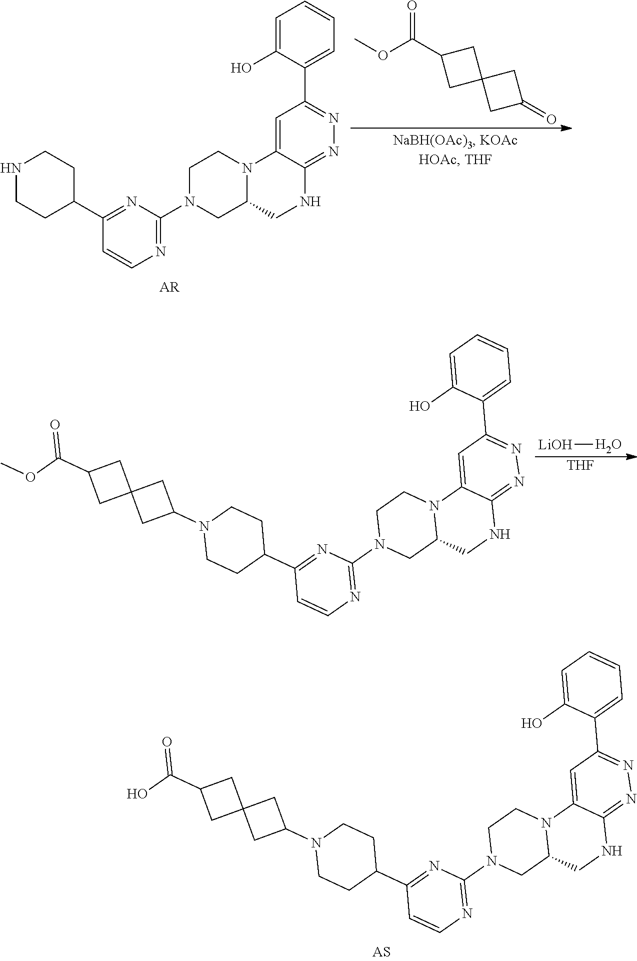

Step 1-(R)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidin-1-yl)spiro[3.3]heptane-2-carboxylate. To a solution of 2-[(1OR)-12-[4-(4-piperidyl)pyrimidin-2-yl]-1,5,6,8,12-pentazatricyclo[8.4.0.0^{2,7}]tetradeca-2,4,6-trien-4-yl]phenol (100 mg, 225 umol, Intermediate AR) in THF (4 mL) was added KOAc (22.1 mg, 225 umol) and the mixture was stirred for 30 minutes. Then methyl 2-oxospiro[3.3]heptane-6-carboxylate (56.8 mg, 337 umol, CAS #113840-98-4) and HOAc (27 mg, 450 umol, 25.7 uL) was added and the mixture was stirred for 30 minutes. Next, NaBH(OAc)₃ (119 mg, 562 umol) was added and the mixture was stirred at 25° C. for 13 hours. The mixture was concentrated under reduce pressure to give a residue. The crude product was purification by reversed-phase flash (0.1% FA condition) to give the title compound: (95 mg, 70% yield) as a white solid. ¹H NMR (400 MHz, DMSO-d6) δ ppm: 8.31 (d, J=5.2 Hz, 1H), 8.15 (s, 2H), 7.95-7.91 (m, 1H), 7.40 (d, J=3.3 Hz, 1H), 7.30 (s, 1H), 7.24-7.18 (m, 1H), 6.89-6.84 (m, 1H), 6.62 (d, J=5.2 Hz, 1H), 4.79-4.73 (m, 1H), 4.18 (d, J=12.4 Hz, 1H), 3.62-3.59 (m, 5H), 3.58 (s, 3H), 3.15 (t, J=4.0 Hz, 2H), 3.05-3.02 (m, 1H), 2.41 (d, J=8.4 Hz, 2H), 2.27-2.17 (m, 2H), 2.13-2.00 (m, 3H), 1.86-1.74 (m, 9H), 1.73-1.63 (m, 2H); LC-MS (ESI⁺) m/z 597.2 (M+H)⁺.

Step 2-(R)-6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidin-1-yl)spiro[3.3]heptane-2-carboxylic acid. To a solution of methyl 2-[4-[2-[(1OR)-4-(2-hydroxyphenyl)-1,5,6,8,12-pentazatricyclo[8.4.0.0^{2,7}]tetradeca-2,4,6-trien-12-yl]pyrimidin-4-yl]-1-piperidyl]spiro[3.3]heptane-6-carboxylate (0.095 g, 159 umol) in THF (1 mL) was added LiOH—H₂O (2 M, 478 uL). The mixture was stirred at 25° C. for 12 hours. On completion, the mixture was concentrated under reduce pressure to give the title compound (150 mg) as a white solid. LC-MS (ESI*) m/z 583.3 (M+H)⁺.

### (R)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidine-1-carboxylate (Intermediate AT) and (S)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidine-1-carboxylate (Intermediate AU)

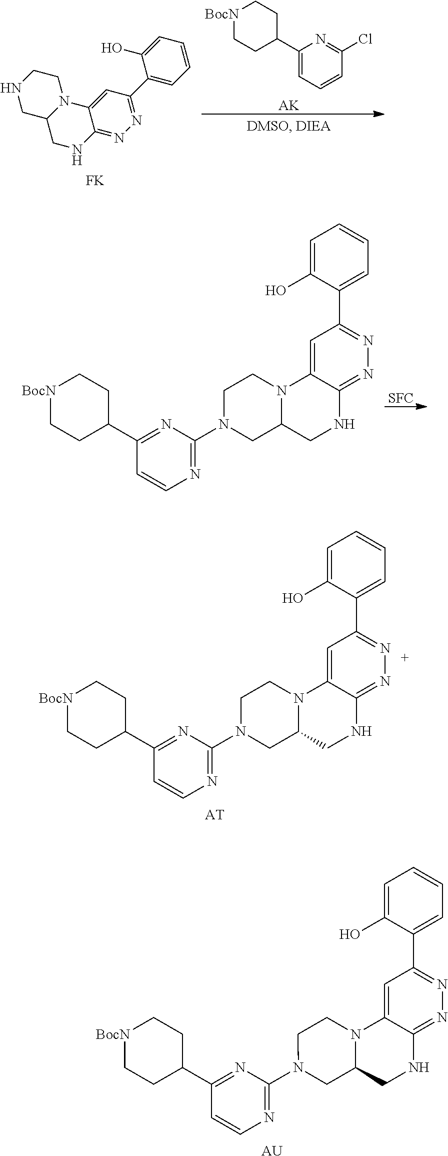

Step 1-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidine-1-carboxylate. To a solution of 2-(1,5,6,8,12-pentazatricyclo[8.4.0.0^{2,7}]tetradeca-2,4,6-trien-4-yl)phenol (720 mg, 2.25 mmol, HCl, Intermediate FK) and tert-butyl 4-(2-chloropyrimidin-4-yl)piperidine-1-carboxylate (0.67 g, 2.25 mmol, Intermediate AK) in DMSO (15 mL) was added DIEA (1.45 g, 11.3 mmol, 1.96 mL). The mixture was stirred at 60° C. for 12 hours. On completion, the mixture was filtered under reduce pressure to give a residue. The crude product was purified by reversed-phase Flash (0.1% FA condition) to give the title compound (270 mg, 21% yield) as a white solid. ¹H NMR (400 MHz, DMSO-d6) δ ppm: 8.32 (d, J=5.2 Hz, 1H), 7.98-7.89 (m, 1H), 7.40 (d, J=3.6 Hz, 1H), 7.30 (s, 1H), 7.25-7.16 (m, 1H), 6.89-6.84 (m, 1H), 6.64 (d, J=5.2 Hz, 1H), 4.76 (d, J=13.2 Hz, 1H), 4.18 (d, J=12.4 Hz, 1H), 4.10-3.97 (m, 2H), 3.66-3.56 (m, 1H), 3.38 (s, 1H), 3.29 (s, 1H), 3.27-3.22 (m, 1H), 3.20-3.10 (m, 1H), 2.97 (t, J=12.4 Hz, 1H), 2.90-2.62 (m, 4H), 1.89-1.76 (m, 2H), 1.63-1.49 (m, 2H), 1.43-1.39 (m, 9H); LC-MS (ESI⁺) m/z 545.5 (M+H)⁺.

Step 2-(R)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidine-1-carboxylate and (S)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidine-1-carboxylate. Tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidine-1-carboxylate was separated by SFC (column: DAICEL CHIRALCEL OD(250 mm×50 mm,10 um); mobile phase: [0.1% NH₃H₂O MEOH]; B %: 50%-50%, 3; 100 min) to give (S)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidine-1-carboxylate (130 mg, 90% purity, SFC retention time=1.603 min) as a white solid (¹H NMR (400 MHz, DMSO-d6) δ ppm: 8.32 (d, J=5.2 Hz, 1H), 7.97-7.90 (m, 1H), 7.40 (d, J=3.2 Hz, 1H), 7.30 (s, 1H), 7.25-7.17 (m, 1H), 6.89-6.84 (m, 1H), 6.64 (d, J=5.2 Hz, 1H), 4.76 (d, J=13.2 Hz, 1H), 4.18 (d, J=12.4 Hz, 1H), 4.09-4.00 (m, 2H), 3.66-3.58 (m, 1H), 3.30-3.22 (m, 1H), 3.19-3.10 (m, 1H), 3.01-2.92 (m, 1H), 2.91-2.75 (m, 3H), 2.75-2.64 (m, 1H), 2.35-2.29 (m, 1H), 1.90-1.77 (m, 3H), 1.62-1.47 (m, 3H), 1.41 (d, J=3.1 Hz, 9H); LC-MS (ESI⁺) m/z 545.2 (M+H)⁺) and (R)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidine-1-carboxylate (100 mg, 99% purity, SFC retention time=1.169 min) as a white solid. ¹H NMR (400 MHz, DMSO-d6) δ 8.33 (d, J=5.2 Hz, 1H), 7.98-7.89 (m, 1H), 7.41 (d, J=3.2 Hz, 1H), 7.31 (s, 1H), 7.25-7.18 (m, 1H), 6.91-6.83 (m, 2H), 6.65 (d, J=5.2 Hz, 1H), 4.77 (d, J=12.8 Hz, 2H), 4.19 (d, J=12.4 Hz, 1H), 4.12-3.98 (m, 2H), 3.63 (d, J=11.2 Hz, 1H), 3.30-3.24 (m, 1H), 3.19-3.08 (m, 1H), 2.97 (t, J=3.6 Hz, 1H), 2.90-2.64 (m, 5H), 1.83 (d, J=11.6 Hz, 2H), 1.62-1.49 (m, 2H), 1.42 (s, 9H); LC-MS (ESI⁺) m/z 545.2 (M+H)⁺. Absolute stereochemistry was arbitrarily assigned.

### (S)-2-(8-(4-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (IntermediateAV)

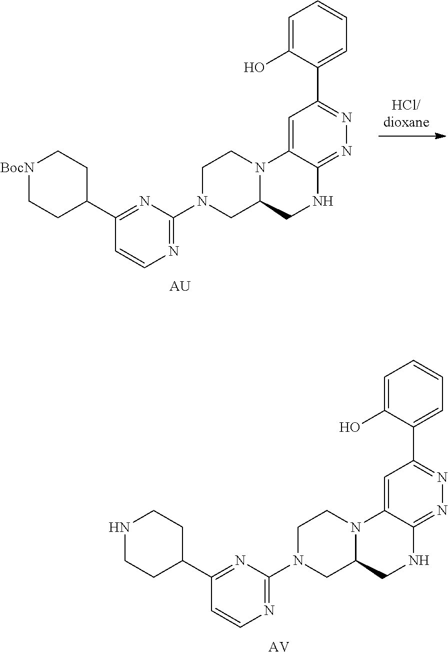

To a solution of tert-butyl 4-[2-[(10S)-4-(2-hydroxyphenyl)-1,5,6,8,12-pentazatricyclo[8.4.0.0^{2,7}]tetradeca-2,4,6-trien-12-yl]pyrimidin-4-yl]piperidine-1-carboxylate (0.13 g, 239 umol, Intermediate AU) in DCM (2 mL) was added HCl/dioxane (4 M, 0.8 mL). The mixture was stirred at 25° C. for 12 hours. On completion, the mixture was concentrated under reduce pressure to give the title compound (130 mg, HCl) as a white solid. LC-MS (ESI⁺) m/z 445.2 (M+H)⁺.

### (S)-6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidin-1-yl)spiro[3.3]heptane-2-carboxylic acid (Intermediate AW)

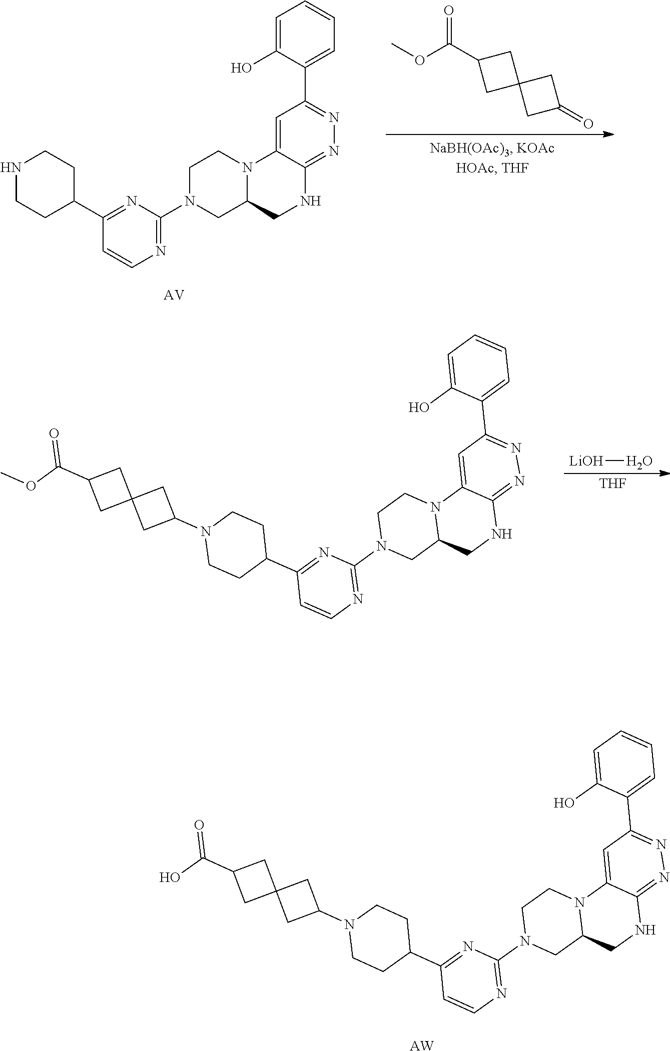

Step 1-(S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidin-1-yl)spiro[3.3]heptane-2-carboxylate. To a solution of 2-[(10S)-12-[4-(4-piperidyl)pyrimidin-2-yl]-1,5,6,8,12-pentazatricyclo[8.4.0.0^{2,7}]tetradeca-2,4,6-trien-4-yl]phenol (0.11 g, 248 umol, Intermediate AV) in THF (4 mL) was added KOAc (24.3 mg, 248 umol) was stirred for 30 minutes. Then methyl 2-oxospiro[3.3]heptane-6-carboxylate (62.4 mg, 371 umol, CAS #1138480-98-4) and HOAc (29.7 mg, 495 umol, 28.30 uL) was added and the mixture was stirred for 30 minutes. Finally, NaBH(OAc)₃ (131 mg, 619 umol) and the mixture was stirred at 25° C. for 13 hours. On completion, the mixture was concentrated under reduce press to give a residue. The crude product was purification by reversed-phase Flash (0.1% FA condition) to give the title compound (75 mg, 50% yield) as a white solid. ¹H NMR (400 MHz, DMSO-d₆): δ=8.31 (d, J=5.2 Hz, 1H), 8.17 (s, 1H), 7.94 (d, J=8.0 Hz, 1H), 7.41 (d, J=3.2 Hz, 1H), 7.31 (s, 1H), 7.25-7.19 (m, 1H), 6.90-6.84 (m, 2H), 6.62 (d, J=5.2 Hz, 1H), 4.77 (d, J=12.4 Hz, 2H), 4.19 (d, J=12.4 Hz, 1H), 3.64 (d, J=3.2 Hz, 1H), 3.61 (s, 2H), 3.59 (s, 3H), 3.17 (s, 1H), 3.15 (t, J=4.0 Hz, 2H), 3.12 (s, 1H), 3.08-3.02 (m, 2H), 3.01-2.94 (m, 1H), 2.91-2.81 (m, 2H), 2.41 (d, J=8.4 Hz, 2H), 2.26-2.19 (m, 2H), 2.14-2.08 (m, 2H), 2.07-1.99 (m, 1H), 1.85-1.76 (m, 4H), 1.74-1.64 (m, 2H); LC-MS (ESI⁺) m/z 597.3 (M+H)⁺.

Step 2-(S)-6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperidin-1-yl)spiro[3.3]heptane-2-carboxylic acid. To a solution of methyl 2-[4-[2-[(10S)-4-(2-hydroxyphenyl)-1,5,6,8,12-pentazatricyclo[8.4.0.0^{2,7}]tetradeca-2,4,6-trien-12-yl]pyrimidin-4-yl]-1-piperidyl]spiro[3.3]heptane-6-carboxylate (0.07 g, 117 umol) in THF (1 mL) was added LiOH—H₂O (2 M, 352 uL). Then the mixture was stirred at 25° C. for 12 hours. On completion, the mixture was concentrated under reduce pressure to give the title compound (110 mg) as a white solid. LC-MS (ESI⁺) m/z 583.2 (M+H)⁺.

### (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (Intermediate AX)

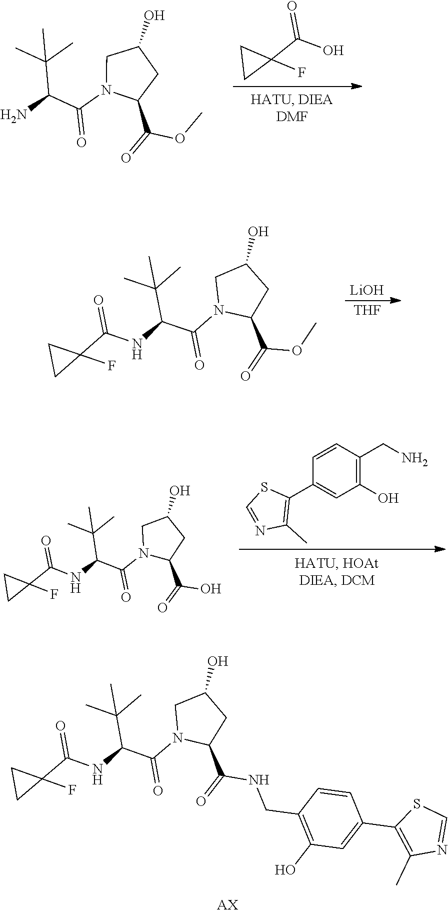

Step 1-(2S,4R)-methyl 1-((S)-2-(1-fluorocyclopropanecarboxamido)-3,3-dimethylbutanoyl)-4-hydroxypyrrolidine-2-carboxylate. To a solution of methyl (2S,4R)-1-[(2S)-2-amino-3,3-dimethyl-butanoyl]-4-hydroxy-pyrrolidine-2-carboxylate (129 g, 437.62 mmol, CAS #1024616-23-6) in DMF (1500 mL) was added DIEA (169.68 g, 1.31 mol, 228.67 mL), HATU (216.32 g, 568.91 mmol),1-fluorocyclopropanecarboxylic acid (50.10 g, 481.39 mmol, CAS #137081-41-5). The mixture was then stirred at 25° C. for 2 hours. On completion, the reaction mixture was with H₂O (500 mL) at 25° C., and then diluted with ethyl acetate (500 mL) and extracted with ethyl acetate (500 mL×3). The combined organic layers were washed with sat. NaCl (500 mL), dried over Na₂SO₄, filtered and concentrated under reduced pressure to give a residue. The residue was purified by column chromatography (SiO₂, Petroleum ether/Ethyl acetate=2/1) to give title compound (152 g, 99% yield) as a yellow oil. LC-MS (ESI⁺) m/z 345.0 (M+H)⁺.

Step 2-(2S,4R)-1-((S)-2-(1-fluorocyclopropanecarboxamido)-3,3-dimethylbutanoyl)-4-hydroxypyrrolidine-2-carboxylic acid. To a solution of methyl (2S,4R)-1-[(2S)-2-[(1-fluorocyclopropanecarbonyl)amino]-3,3-dimethyl-butanoyl]-4-hydroxy-pyrrolidine-2-carboxylate (30 g, 87.11 mmol) in THF (190 mL) and H₂O (44 mL) was added LiOH·H₂O (10.97 g, 261.34 mmol). The mixture was stirred at 25° C. for 2 hours. On completion, the reaction mixture was add 2M HCl until pH=1, then the solution was extracted with ethyl acetate (300 mL×3). The combined organic layers were washed with sat. NaCl (500 mL), dried over Na₂SO₄, filtered and concentrated under reduced pressure to give the title compound (31 g) was a white solid. LC-MS (ESI⁺) m/z 331.4 (M+H)⁺.

Step 3-(2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide. To a solution of 2-(aminomethyl)-5-(4-methylthiazol-5-yl)phenol (5 g, 19.47 mmol, HCl, CAS #1448190-11-1) and (2S,4R)-1-[(2S)-2-[(1-fluorocyclopropanecarbonyl)amino]-3,3-dimethyl-butanoyl]-4-hydroxy-pyrrolidine-2-carboxylic acid (8.36 g, 25.32 mmol) in DCM (200 mL) was added HATU (8.15 g, 21.42 mmol), HOAt (3.45 g, 25.32 mmol, 3.54 mL) and DIEA (12.58 g, 97.37 mmol, 16.96 mL) at −10° C. The mixture was then stirred at −10° C. for 1 hour. On completion, the reaction mixture was concentrated under reduced pressure to give a residue. The residue was then purified by column chromatography (SiO₂, Petroleum ether/Ethyl acetate=2/1) to give title compound (8.1 g, 80% yield) as a yellow solid. LC-MS (ESI⁺) m/z 533.2 (M+H)⁺.

### (2S,4R)-1-((S)-2-(1-fluorocyclopropanecarboxamido)-3,3-dimethylbutanoyl)-4-hydroxy-N-(4-(4-methylthiazol-5-yl)-2-(4-oxobutoxy)benzyl)pyrrolidine-2-carboxamide (Intermediate AY)

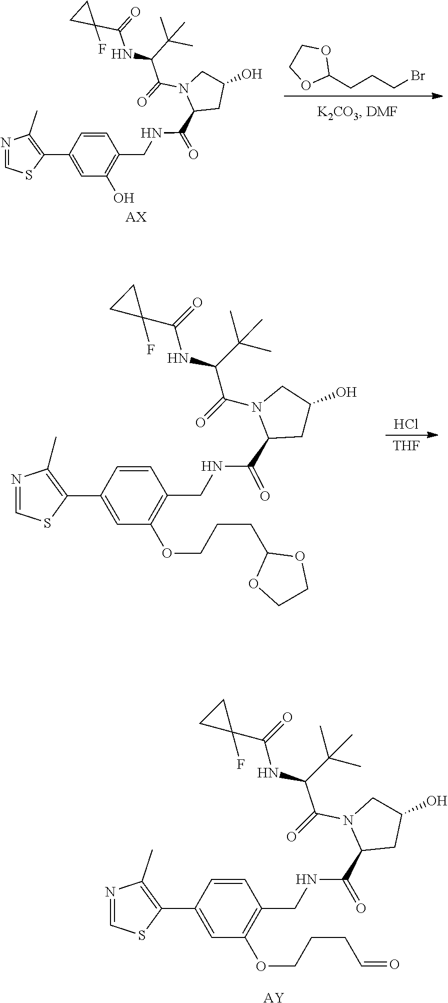

Step 1-(2S,4R)—N-(2-(3-(1,3-dioxolan-2-yl)propoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide. To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (400 mg, 751 mmol, Intermediate AX) in DMF (2 mL) was added K₂CO₃ (311 mg, 2.25 mmol) and 2-(3-bromopropyl)-1,3-dioxolane (146 mg, 751 umol, CAS #62563-07-9). Then the mixture was stirred at 80° C. for 12 hours. On completion, the residue was diluted with water (2 mL) and extracted with ethyl acetate (3×2 mL). The combined organic layers were washed with brine (30 mL), dried over Na₂SO₄, filtered and concentrated under reduced pressure to give the title compound (550 mg). LC-MS (ESI+) m/z 647.2 (M+H)⁺.

Step 2-(2S,4R)-1-((S)-2-(1-fluorocyclopropanecarboxamido)-3,3-dimethylbutanoyl)-4-hydroxy-N-(4-(4-methylthiazol-5-yl)-2-(4-oxobutoxy)benzyl)pyrrolidine-2-carboxamide. To a solution of (2S,4R)—N-(2-(3-(1,3-dioxolan-2-yl)propoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (550 mg, umol) in THF (2 mL) was added HCl (1 M, 2.13 mL). The mixture was then stirred at 50° C. for 12 hours. On completion, the reaction mixture was filtered and concentrated under reduced pressure to give a residue. The crude product was purified by reversed-phase HPLC (0.1% FA condition) to give the title compound (190 mg, 34% yield) as white solid. LC-MS (ESI+) m/z 603.5 (M+H)⁺.

### (2S,4R)-1-((S)-2-(1-fluorocyclopropane-1-carboxamido)-3,3-dimethylbutanoyl)-4-hydroxy-N-(4-(4-methylthiazol-5-yl)-2-(2-oxoethoxy)benzyl)pyrrolidine-2-carboxamide (Intermediate AZ)

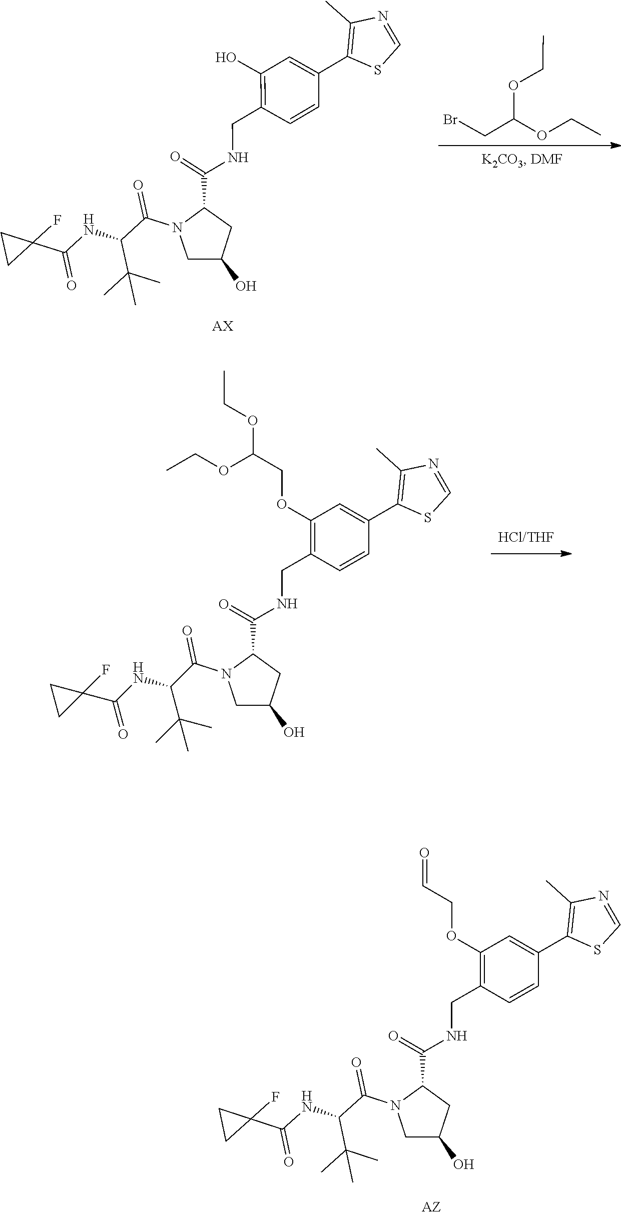

Step 1-(2S,4R)—N-[[2-(2,2-diethoxyethoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide. To a solution of (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (300 mg, 563 umol, Intermediate AX) in DMF (2 mL) was added K₂CO₃ (389 mg, 2.82 mmol) and 2-bromo-1,1-diethoxy-ethane (444 mg, 2.25 mmol, 338 uL). The mixture was then stirred at 80° C. for 16 hours. The reaction mixture was diluted with H₂O (15 mL) and extracted with ethyl acetate (30 mL×3). The combined organic layers were washed with brine (15 mL×3), dried over Na₂SO₄, filtered and concentrated under reduced pressure to give the title compound (300 mg) as a yellow solid. LC-MS (ESI⁺) m/z 649.2 (M+H)⁺.

Step 2-2S,4R)-1-((S)-2-(1-fluorocyclopropane-1-carboxamido)-3,3-dimethylbutanoyl)-4-hydroxy-N-(4-(4-methylthiazol-5-yl)-2-(2-oxoethoxy)benzyl)pyrrolidine-2-carboxamide. To a solution of (2S,4R)—N-[[2-(2,2-diethoxyethoxy)-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide (300 mg, 462 umol) in THF (2 mL) was added HCl (1 M, 462.40 uL). The mixture was then stirred at 50° C. for 12 hours. On completion, the reaction mixture was concentrated under reduced pressure to give a yellow oil. The oil was purified by prep-HPLC (FA condition) to give the title compound (100 mg, 37% yield) as a white solid. LC-MS (ESI⁺) m/z 575.2 (M+H)⁺.

### (S)-2-(8-(5-(piperazin-1-yl)pyrimidin-2-yl)-6,6a,7,8,9,10-hexahydro-5H-pyrazino[1′,2′:4,5]pyrazino[2,3-c]pyridazin-2-yl)phenol (Intermediate EA)

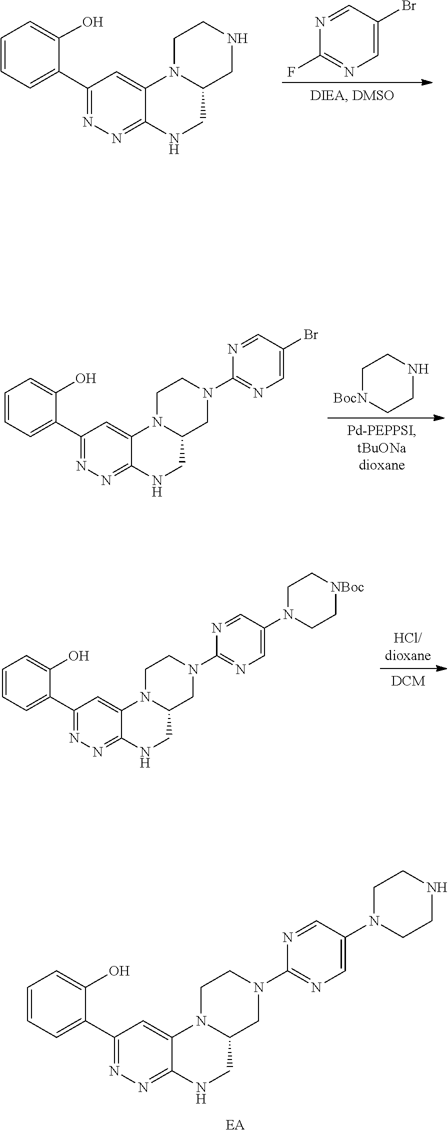 [SMILES: Oc1ccccc1-c1cc2c(nn1)NC[C@H]1CNCCN21 ; Fc1ncc(Br)cn1 ; Oc1ccccc1-c1cc2c(nn1)NC[C@H]1CN(c3ncc(Br)cn3)CCN21 ; CC(C)(C)OC(=O)N1CCNCC1 ; CC(C)(C)OC(=O)N1CCN(c2cnc(N3CCN4c5cc(-c6ccccc6O)nnc5NC[C@H]4C3)nc2)CC1 ; Oc1ccccc1-c1cc2c(nn1)NC[C@H]1CN(c3ncc(N4CCNCC4)cn3)CCN21]

Step 1 (S)-2-(8-(5-bromopyrimidin-2-yl)-6,6a,7,8,9,10-hexahydro-5H-pyrazino[1′,2′:4,5]pyrazino[2,3-c]pyridazin-2-yl)phenol. To a solution of 2-[(10R)-1,5,6,8,12-pentazatricyclo[8.4.0.02,7]tetradeca-2,4,6-trien-4-yl]phenol (750 mg, 2.35 mmol, Intermediate FF) in DMSO (10 mL) was added DIEA (1.52 g, 11.7 mmol) and 5-bromo-2-fluoro-pyrimidine (622.59 mg, 3.52 mmol) at 25° C. Then the reaction was stirred at 60° C. for 12 hours. On completion, the reaction mixture was quenched with water (50 mL) and extracted by ethyl acetate (3×50 mL). The extracts were washed by brine (50 mL) and dried over anhydrous sodium sulfate, filtered and concentrated in vacuo to get the crude residue. The residue was purified by column chromatography (SiO₂, Petroleum ether/Ethyl acetate=1/1 to DCM: MeOH=20/1) to give the title compound (480 mg, 46% yield) as pink solid. LC-MS (ESI⁺) m z 440.3. (M+H)⁺.

Step 2 (S)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazine-1-carboxylate. To a solution of 2-[(10S)-12-(5-bromopyrimidin-2-yl)-1,5,6,8,12-pentazatricyclo[8.4.0.02,7]tetradeca-2,4,6-trien-4-yl]phenol (480 mg, 1.09 mmol) in dioxane (8 mL) was added tert-butyl piperazine-1-carboxylate (406 mg, 2.18 mmol), Pd-PEPPSI(TM)-IPENT catalyst (130 mg, 164 umol) and tBuONa (2 M, 1.64 mL) at 25° C. The suspension was degassed and purged with N₂ three times. The reaction was stirred at 100° C. for 8 hours. On completion, the reaction mixture was quenched with water (20 mL) and extracted by DCM (3×20 mL). The extracts were washed by brine (30 mL) and dried over anhydrous sodium sulfate, filtered and concentrated in vacuo to get the crude residue. The residue was purified by column chromatography (SiO₂, Petroleum ether/Ethyl acetate=1/1 to DCM: MeOH=15:1) to give the title compound (490 mg, 68% yield) as red solid. LC-MS (ESI⁺) m/z 470.2. (M+H)⁺.

Step 3 (S)-2-(8-(5-(piperazin-1-yl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl 4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]piperazine-1-carboxylate (490 mg, 898 umol) in DCM (6 mL) was added HCl/dioxane (5 M, 1.5 mL) at 25° C., then the reaction was stirred at 25° C. for 1 hour. On completion, the reaction mixture was filtered and concentrated in vacuo to give the title compound (600 mg) as brown solid. LC-MS (ESI⁺) m/z 446.4. (M+H)⁺.

### (S)-6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.3]heptane-2-carboxylic acid (Intermediate EB)

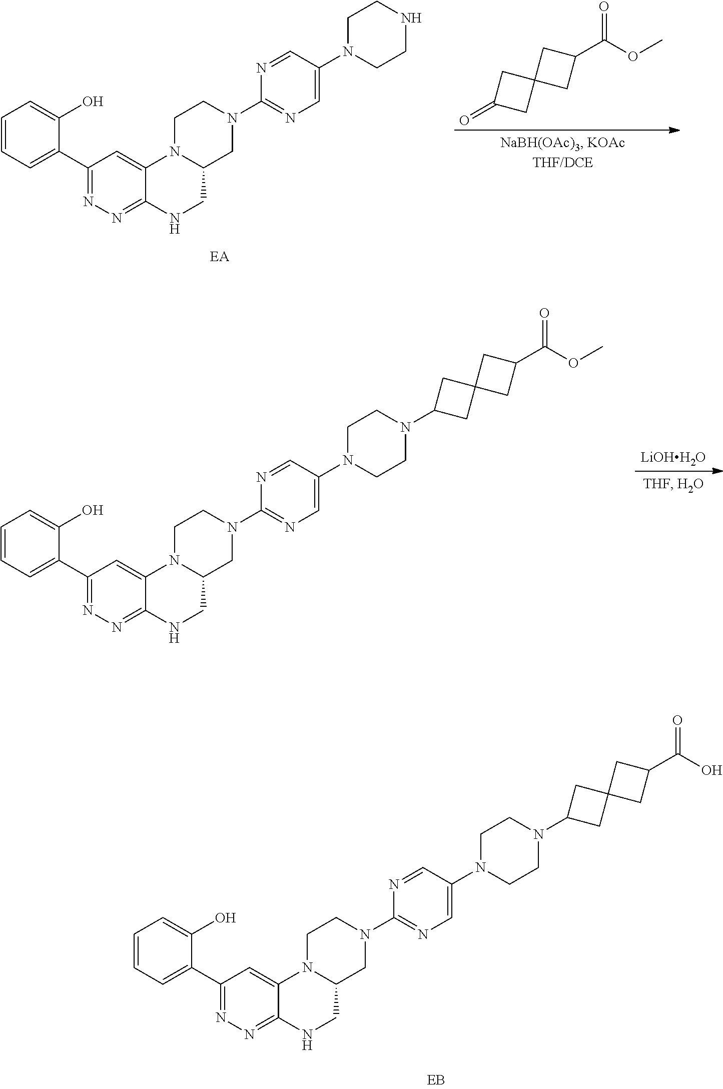 [SMILES: Oc1ccccc1-c1cc2c(nn1)NC[C@H]1CN(c3ncc(N4CCNCC4)cn3)CCN21 ; COC(=O)C1CC2(CC(=O)C2)C1 ; COC(=O)C1CC2(C1)CC(N1CCN(c3cnc(N4CCN5c6cc(-c7ccccc7O)nnc6NC[C@H]5C4)nc3)CC1)C2 ; "LiOH·H2O" ; O=C(O)C1CC2(C1)CC(N1CCN(c3cnc(N4CCN5c6cc(-c7ccccc7O)nnc6NC[C@H]5C4)nc3)CC1)C2]

Step 1-(S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.3]heptane-2-carboxylate. To a solution 2-[(10S)-12-(5-piperazin-1-ylpyrimidin-2-yl)-1,5,6,8,12-pentazatricyclo[8.4.0.0^{2,7}]tetradeca-2,4,6-trien-4-yl]phenol (600 mg, 1.24 mmol, HCl), Intermediate EA in THF (10 mL) and DCE (2 mL) was added AcOK (367 mg, 3.73 mmol) and the reaction mixture was stirred at 25° C. for 0.5 hour. Then AcOH (299 mg, 4.98 mmol) and methyl 2-oxospiro[3.3]heptane-6-carboxylate (419 mg, 2.49 mmol, CAS #1138480-984) was added at 25° C., then the mixture was stirred at 25° C. for 1 hour. Next, NaBH(OAc)₃ (528 mg, 2.49 mmol) was added at 0° C., then the reaction was stirred at 25° C. for 1 hour. On completion, the reaction mixture was quenched with MeOH (10 mL), filtered and concentrated in vacuo to get the crude residue. The residue was purified by column chromatography (SiO₂, DCM: MeOH=100/1 to 10:1) to give the title compound (320 mg, 33% yield) as yellow solid. LC-MS (ESI⁺) m/z 598.5. (M+H)⁺.

Step 2-(S)-6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.3]heptane-2-carboxylic acid. To a solution of methyl 2-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]piperazin-1-yl]spiro[3.3]heptane-6-carboxylate (320 mg, 535 umol) in THF (3 mL) and H₂O (3 mL) was added LiOH·H₂O (112 mg, 2.68 mmol) at 25° C., then the reaction was stirred at 25° C. for 2 hours. On completion, the reaction mixture was added HCl (1N) until pH=3, filtered and lyophilize to give the title compound (540 mg) as brown solid.

### 2-(3-methoxyisoxazol-5-yl)-3-methylbutanoic acid (Intermediate EC)

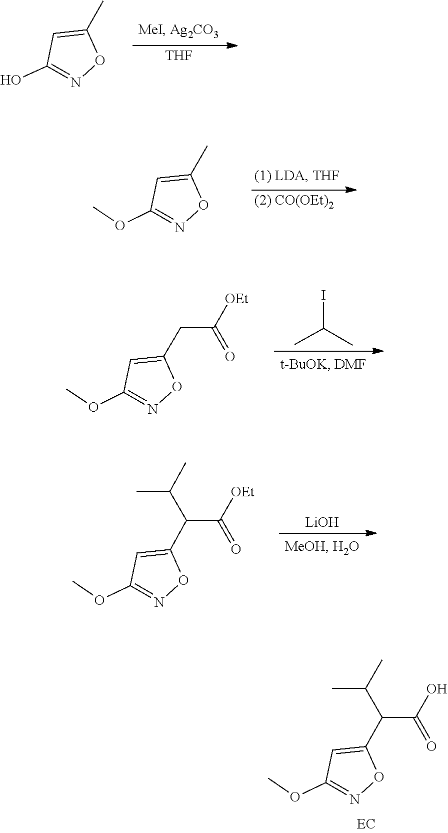

Step 1-3-Methoxy-5-methylisoxazole. To a solution of 5-methylisoxazol-3-ol (70 g, 706.44 mmol, CAS #10004-44-1) in THF (500 mL) was added CH₃I (989 mmol, 61.57 mL), followed by addition of Ag₂CO₃ (777 mmol) in portions at 25° C. The mixture was then stirred at 60° C. for 12 h. On completion, the reaction mixture was filtered and the filtered liquor was concentrated under reduced pressure to give the title compound (78 g) as yellow oil. LC-MS (ESI, m/z): [M+1]⁺=114.2.

Step 2-Ethyl 2-(3-methoxyisoxazol-5-yl)acetate. To a solution of 3-methoxy-5-methyl-isoxazole (78 g, 690 mmol) in THF (700 mL) was added LDA (2.0 M, 413.74 mL) dropwise at −70° C. for 1 hr. Then diethyl carbonate (896.44 mmol, 108.61 mL) in THF (100 mL) was added slowly into the solution above at −70° C., and the reaction solution was stirred for another 3 hr. The reaction mixture was quenched with saturated ammonium chloride aqueous solution (200 mL) dropwise at −70° C. The suspension mixture was filtered to get the filtrate and extracted with ethyl acetate (300 mL×3). The combined organic layers were washed by saturated brine (300 mL) and dried over anhydrous sodium sulfate, filtered and concentrated in vacuo to get the crude residue. The crude product was purified by reversed-phase HPLC (column: 120 g Flash Coulmn; Welch Ultimate XB_C18 20-40 μm; 120 A; mobile phase: [water(0.1% FA)-ACN];B %: 5-70% 0 min;% min) to give the title compound (32 g, 22% yield) as a yellow oil. LC-MS (ESI, m/z): [M+1]⁺=186.1.

Step 3-Ethyl 2-(3-methoxyisoxazol-5-yl)-3-methylbutanoate. To a solution of ethyl 2-(3-methoxyisoxazol-5-yl)acetate (28 g, 151.21 mmol) in DMF (300 mL) was added t-BuOK (25.45 g, 226.81 mmol), followed by slow addition of 2-iodopropane (25.70 g, 151.21 mmol, 15.12 mL). The mixture was then stirred at 0° C. for 2 hr. On completion, the reaction mixture was quenched with saturated aqueous ammonium chloride solution (100 mL) and ethyl acetate (50 mL). The suspension mixture was filtered to get the filtrate and extracted with ethyl acetate (300 mL×3). The combined organic layers were washed by saturated brine (100 mL×3) and dried over anhydrous sodium sulfate, filtered and concentrated in vacuo to give the title compound (30 g, 87% yield) as a yellow oil. LC-MS (ESI, m/z): [M+1]⁺=228.2.

Step 4-2-(3-Methoxyisoxazol-5-yl)-3-methylbutanoic acid. Ethyl 2-(3-methoxyisoxazol-5-yl)-3-methyl-butanoate (1 g, 4.4 mmol) was dissolved in MeOH (4 mL), THF (4 mL) and H₂O (4 mL). Then LiOH—H₂O (369.3 mg, 8.8 mmol) was added in portions at 0° C., and the mixture was stirred at 25° C. for 3 hr. On completion, the reaction mixture was quenched by slow addition of 1 N HCl to at 0° C. with stirring until the pH=2.0. The mixture was then diluted with water (20 ml) and extracted with ethyl acetate (30 mL×3). The combined organic layers were washed by saturated brine (10 mL×2) and dried over anhydrous sodium sulfate, filtered and concentrated under reduced pressure to give the title compound (765 mg, 88% yield) as a yellow oil. ¹H NMR (400 MHz, MeOD-d₄) δ=6.00 (s, 1H), 6.00 (s, 3H), 3.47 (d, 1H, J=8.8), 1.03 (m, 1H), 0.91 (d, 3H, J=6.8), 0.93 (d, 3H, J=6.4). LC-MS (ESI, m/z): [M+1]+=200.1.

### (S)-2-(8-(5-(1-(7-azaspiro[3.5]nonan-2-yl)piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate ED)

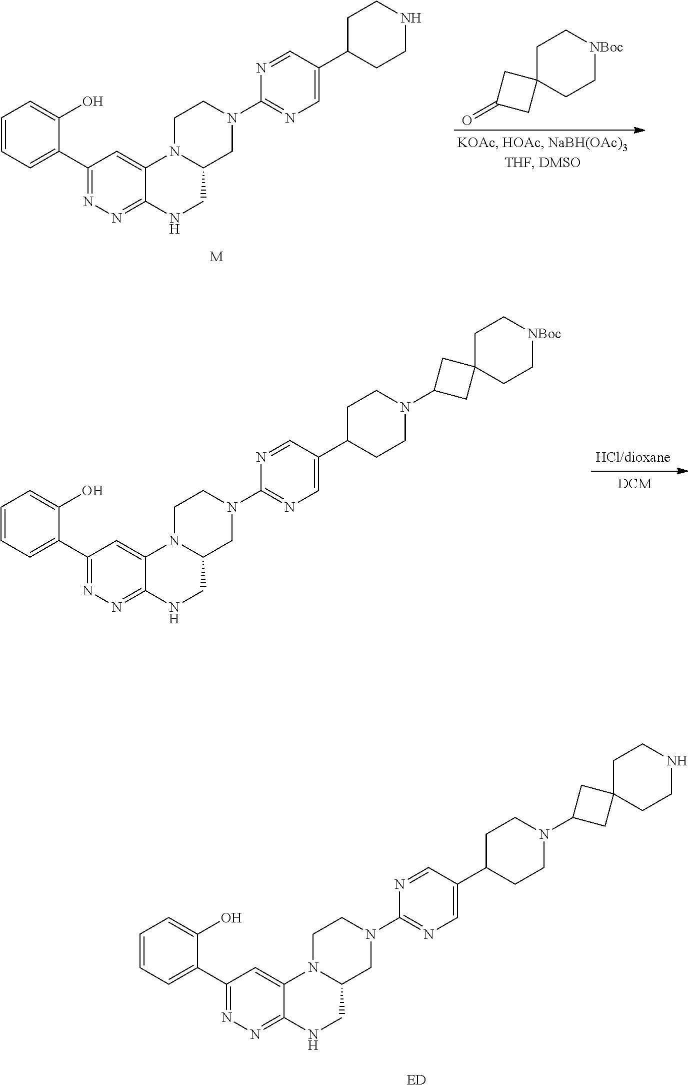

Step 1-(S)-tert-butyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-7-azaspiro[3.5]nonane-7-carboxylate. To a solution of 2-[(10S)-12-[5-(4-piperidyl)pyrimidin-2-yl]-1,5,6,8,12-pentazatricyclo[8.4.0.0^{2,7}]tetradeca-2,4,6-trien-4-yl]phenol (550 mg, 1.24 mmol, Intermediate M) in THF (8 mL) and DMSO (2 mL) was added KOAc (364 mg, 3.71 mmol). After 30 minutes of stirring at 25° C., tert-butyl 2-oxo-7-azaspiro[3.5]nonane-7-carboxylate (592 mg, 2.47 mmol) and HOAc (297 mg, 4.95 mmol, 283 uL) was added. After an additional 30 minutes of stirring, NaBH(OAc)₃ (787 mg, 3.71 mmol) was added and the mixture was stirred at 25° C. for 11 hours. On completion, the mixture was filtered to give a solution. The crude product was purified by reversed-phase Flash (0.1% FA condition) to give the title compound (210 mg, 24% yield) as a white solid. ¹H NMR (400 MHz, DMSO-d₆) δ 8.35 (s, 2H), 7.94 (d, J=8.0 Hz, 1H), 7.40 (d, J=3.6 Hz, 1H), 7.31 (s, 1H), 7.24-7.18 (m, 1H), 6.89-6.84 (m, 2H), 4.76-4.66 (m, 2H), 4.20 (d, J=12.8 Hz, 1H), 3.65-3.58 (m, 1H), 3.28-3.23 (m, 3H), 3.21-3.15 (m, 3H), 3.14-3.08 (m, 1H), 2.95 (d, J=4.0 Hz, 1H), 2.88 (d, J=10.4 Hz, 2H), 2.75 (dd, J=10.8 Hz, J=13.2 Hz, 1H), 2.66-2.60 (m, 1H), 2.43-2.34 (m, 3H), 2.00-1.92 (m, 2H), 1.76-1.67 (m, 4H), 1.62 (t, J=12.0 Hz, 2H), 1.54-1.50 (m, 2H), 1.47 (dd, J=4.4 Hz, J=6.4 Hz, 3H), 1.38 (s, 9H); LC-MS (ESI⁺) m/z 668.2 (M+H)⁺.

Step 2-(S)-2-(8-(5-(1-(7-azaspiro[3.5]nonan-2-yl)piperidin-4-yl)pyrimidin-2-yl)-6,6a,7,8,9,10-hexahydro-5H-pyrazino[1′,2′:4,5]pyrazino[2,3-c]pyridazin-2-yl). To a solution of tert-butyl 2-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]-7-azaspiro[3.5]nonane-7-carboxylate (190 mg, 285 umol) in DCM (5 mL) was added HCl/dioxane (4 M, 2 mL). The mixture was then stirred at 25° C. for 2 hours. On completion, the mixture was concentrated under reduce pressure to give the title compound (220 mg) as a white solid. LC-MS (ESI⁺) m/z 568.6 (M+H)⁺.

### (S)-2-(8-(5-(1-(3-azaspiro[5.5]undecan-9-yl)piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate EE)

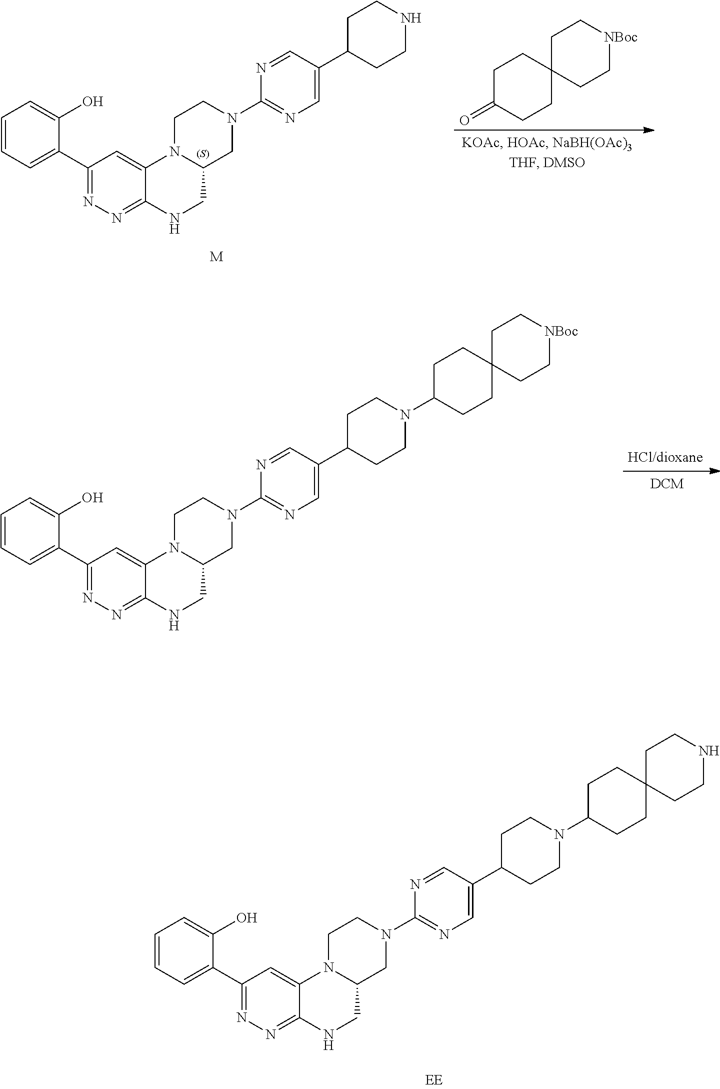

Step 1-tert-butyl 9-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]-3-azaspiro[5.5]undecane-3-carboxylate. To a solution of 2-[(10S)-12-[5-(4-piperidyl)pyrimidin-2-yl]-1,5,6,8,12-pentazatricyclo[8.4.0.0^{2,7}]tetradeca-2,4,6-trien-4-yl]phenol (600 mg, 1.25 mmol, HCl, Intermediate M) in THF (1.5 mL) and DMSO (0.5 mL) was added KOAc (367 mg, 3.74 mmol) and the mixture was stirred 30 minutes. Then tert-butyl 9-oxo-3-azaspiro[5.5]undecane-3-carboxylate (667 mg, 2.49 mmol, CAS #873924-08-4), 4 Å molecular sieves (0.6 g) and HOAc (300 mg, 4.99 mmol, 285 uL) was added and the mixture was stirred for 30 minutes. Next, NaBH(OAc)₃ (793 mg, 3.74 mmol) was added and the mixture was stirred at 25° C. for 12 hours. On completion, the mixture was filtered to give a solution. The crude product was purified by reversed-phase Flash (0.1% HCl condition) to give the title compound (300 mg, 32% yield) as a white solid. ¹H NMR (400 MHz, DMSO-d₆) δ 8.35 (s, 1H), 8.17 (s, 3H), 7.98-7.91 (m, 1H), 7.41 (s, 1H), 7.32 (s, 1H), 7.26-7.18 (m, 1H), 6.86 (d, J=8.0 Hz, 1H), 4.77-4.68 (m, 1H), 4.21 (d, J=11.6 Hz, 1H), 3.29 (s, 9H), 3.19-3.07 (m, 6H), 3.01-2.91 (m, 2H), 2.82-2.73 (m, 1H), 2.68 (s, 1H), 2.34 (s, 1H), 1.84-1.61 (m, 6H), 1.39 (s, 9H), 1.26-1.18 (m, 2H), 1.17-1.03 (m, 2H); LC-MS (ESI⁺) m/z 696.6 (M+H)⁺.

Step 2-(S)-2-(8-(5-(1-(3-azaspiro[5.5]undecan-9-yl)piperidin-4-yl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl 9-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]-3-azaspiro[5.5]undecane-3-carboxylate (250 mg, 359 umol) in DCM (1 mL) was added HCl/dioxane (4 M, 2.00 mL). The mixture was then stirred at 25° C. for 12 hours. On completion, the mixture was concentrated under reduce pressure to give the title compound: (250 mg) as a white solid. LC-MS (ESI⁺) m/z 596.4 (M+H)⁺.

### 2-[(10S)-12-[5-[1-(2-Azaspiro[3.5]nonan-7-yl)-4-piperidyl]pyrimidin-2-yl]-1,5,6,8,12-pentazatricyclo[8.4.0.0^{2,7}]tetradeca-2,4,6-trien-4-yl]phenol (Intermediate EF)

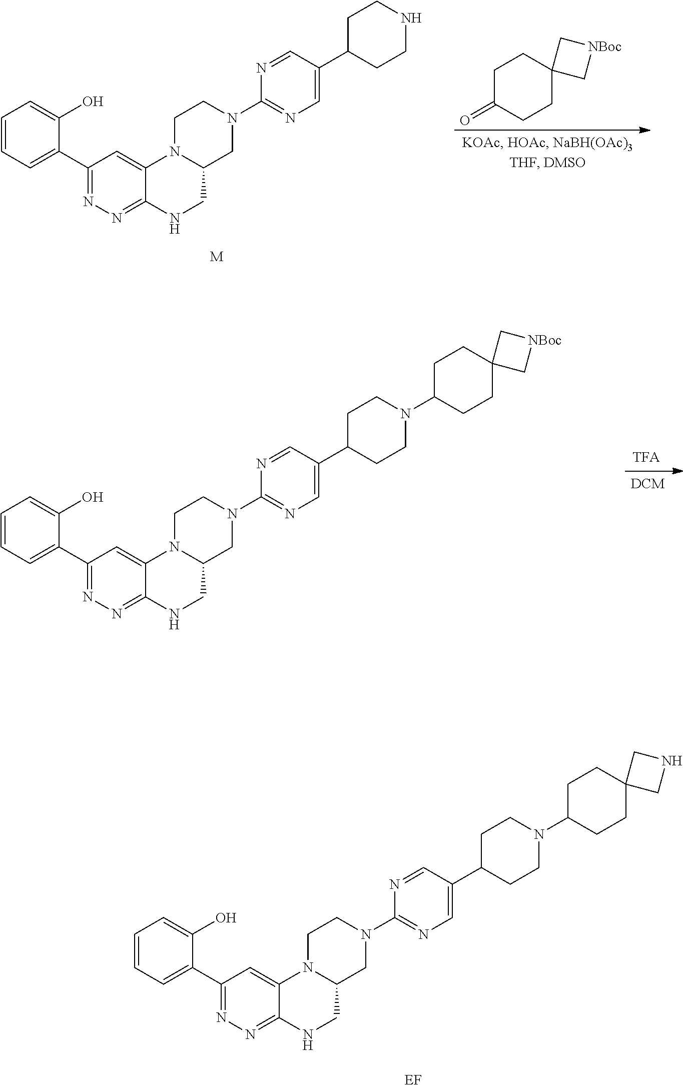

Step 1-(S)-tert-butyl 7-(4-(2-(2-(2-hydroxyphenyl)-6a,7,9,10-tetrahydro-5H-pyrazino[1′,2′ 4,5]pyrazino[2,3-c]pyridazin-8(6H)-yl)pyrimidin-5-yl)piperidin-1-yl)-2-azaspiro[3.5]nonane-2-carboxylate. To a solution of 2-[(10S)-12-[5-(4-piperidyl)pyrimidin-2-yl]-1,5,6,8,12-pentazatricyclo[8.4.0.0^{2,7}]tetradeca-2,4,6-trien-4-yl]phenol (50 mg, 112 umol, Intermediate M) in THF (1 mL) and DMSO (0.5 mL), was added KOAc (33.1 mg, 337 umol) and the mixture was stirred at 25° C. for 0.5 hour. Then tert-butyl 7-oxo-2-azaspiro[3.5]nonane-2-carboxylate (53.8 mg, 225 umol, CAS #1363381-22-9) and AcOH (27 mg, 445 umol) were added in the mixture and the mixture was stirred at 0° C. for 0.5 hour. Finally, NaBH(OAc)₃ (727 mg, 3.43 mmol) was added and the mixture was stirred at 25° C. for 12 hours. On completion, the reaction mixture was purified directly by reversed-phase HPLC (0.1% FA condition) to give the compound (490 mg, 63% yield) as a yellow solid. LC-MS (ESI⁺) m/z 668.3 (M+H)⁺.

Step 2-2-[(10S)-12-[5-[1-(2-azaspiro[3.5]nonan-7-yl)-4-piperidyl]pyrimidin-2-yl]-1,5,6,8,12-pentazatricyclo[8.4.0.0^{2,7}]tetradeca-2,4,6-trien-4-yl]phenol. To a solution of tert-butyl 7-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]-2-azaspiro[3.5]nonane-2-carboxylate (490 mg, 734 umol,) in DCM (5 mL) was added TFA (0.5 mL). The mixture was then stirred at 25° C. for 1 hour. On completion, the reaction mixture was purified directly by prep-HPLC (TFA condition column) to give the title compound (256 mg, 510% yield,) as a yellow solid. LC-MS (ESI⁺) m/z 568.3 (M+H)⁺.

### 2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidine-5-carbaldehyde (Intermediate EG)

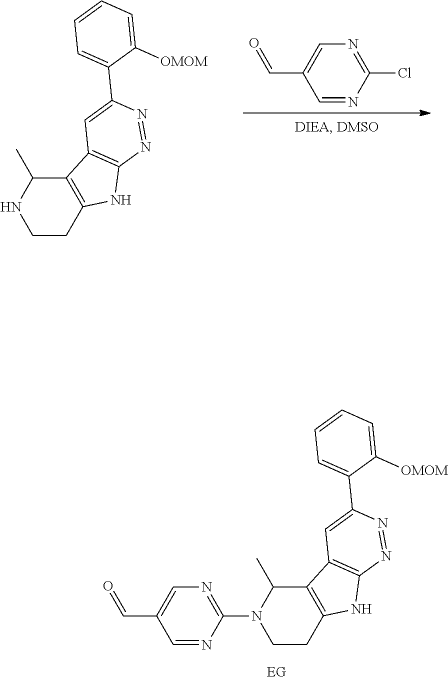

To a solution of 12-[2-(methoxymethoxy)phenyl]-3-methyl-4,8,10,11-tetrazatricyclo[7.4.0.02,7]trideca-1(9),2(7),10,12-tetraene (400 mg, 1.05 mmol, 85% purity, Intermediate Y) in DMSO (6 mL) was added DIEA (406 mg, 3.14 mmol) and 2-chloropyrimidine-5-carbaldehyde (194 mg, 1.36 mmol, CAS #933702-55-7). Then the mixture was stirred at 25° C. for 2 hours. On completion, the reaction mixture was concentrated in vacuo to give a residue. The residue was purified by prep-HPLC (FA condition) to give the title compound (350 mg, 69% yield, FA) as a yellow solid. LC-MS (ESI⁺) m/z 431.3 (M+H)⁺. (2S,4R)-1-((S)-2-(1-fluorocyclopropanecarboxamido)-3,3-dimethylbutanoyl)-4-hydroxy-N-(4-(4-methylthiazol-5-yl)-2-(piperidin-4-yloxy)benzyl)pyrrolidine-2-carboxamide (Intermediate EH)

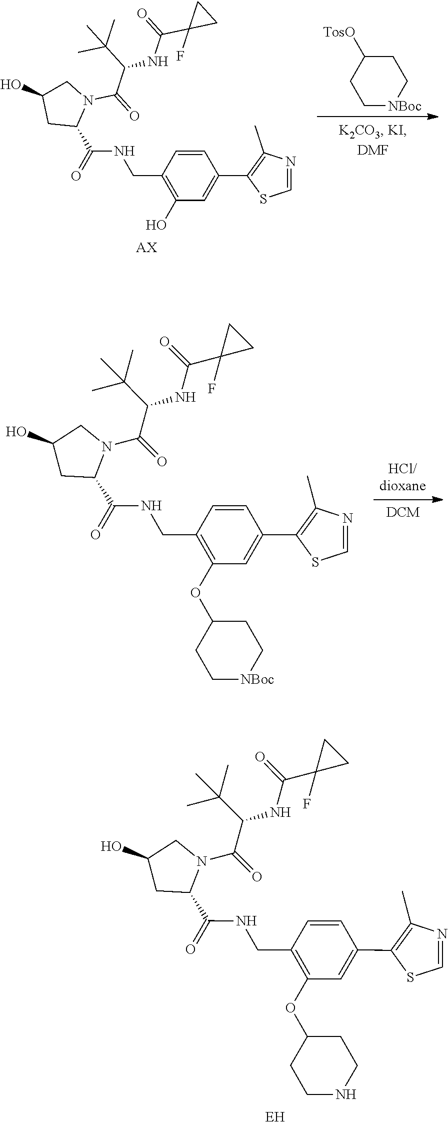

Step 1-tert-butyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)piperidine-1-carboxylate. To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (400 mg, 751 umol, Intermediate AX) in DMF (2 mL) was added K₂CO₃ (726 mg, 5.26 mmol) and KI (124.67 mg, 751 umol). Then tert-butyl 4-(tosyloxy)piperidine-1-carboxylate (1.87 g, 5.26 mmol) was added to the mixture at 25° C. Next, the reaction solution was stirred at 100° C. for 12 hours. On completion, the reaction mixture was diluted with H₂O 10 mL and extracted with EA (20 mL×3). The organic layer was washed with brine 10 mL and dried over Na₂SO₄, concentrated in vacuo to give a residue. The residue was purified by column chromatography (SiO₂, Petroleum ether/Ethyl acetate=1/1 to 1/10) to give the title compound (200 mg, 34% yield) as a white solid. LC-MS (ESI⁺) m/z 716.3 (M+H)⁺.

Step 2-(2S,4R)-1-((S)-2-(1-fluorocyclopropanecarboxamido)-3,3-dimethylbutanoyl)-4-hydroxy-N-(4-(4-methylthiazol-5-yl)-2-(piperidin-4-yloxy)benzyl)pyrrolidine-2-carboxamide. To a mixture tert-butyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)piperidine-1-carboxylate (200 mg, 279 umol) in DCM (3 mL) was added HCl/dioxane (4 M, 0.6 mL) at 25° C. The mixture was stirred at 25° C. for 1 hour. On completion, the reaction mixture was concentrated in vacuo to give the title compound (120 mg) as a yellow oil. LC-MS (ESI⁺) m/z 616.3 (M+H)⁺.

### 2-(4-(2-(3-(2-Hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)acetaldehyde (Intermediate EI)

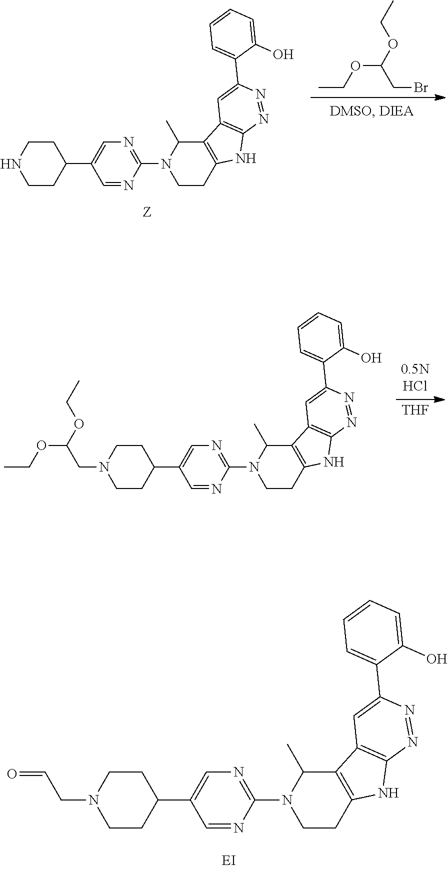

Step 1-2-(6-(5-(1-(2,2-Diethoxyethyl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of 2-[3-methyl-4-[5-(4-piperidyl)pyrimidin-2-yl]-4,8,10,11-tetrazatricyclo[7.4.0.0^{2,7}]trideca-1(9),2(7),10,12-tetraen-12-yl]phenol (460 mg, 1.04 mmol, Intermediate Z) in DMSO (10 mL) was added DIEA (403 mg, 3.13 mmol, 544), 2-bromo-1,1-diethoxy-ethane (821 mg, 4.17 mmol) and KI (86.4 mg, 520 umol). Then the mixture was stirred at 80° C. for 12 hours. On completion, the mixture was diluted with DMF, then the residue was purified by prep-HPLC (TFA) to give title compound (400 mg, 50% yield, TFA) as a yellow oil. LC-MS (ESI⁺) m/z 558.2 (M+H)⁺.

Step 2-2-(4-(2-(3-(2-Hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)acetaldehyde. To a solution of 2-[4-[5-[1-(2,2-diethoxyethyl)-4-piperidyl]pyrimidin-2-yl]-3-methyl-4,8,10,11-tetrazatricyclo[7.4.0.0^{2,7}]trideca-1(9),2(7),10,12-tetraen-12-yl]phenol (300 mg, 537 umol) in H₂O (0.2 mL) was added HCl (2 M, 15.0 mL), then the mixture was stirred at 50° C. for 6 hours. On completion, the reaction mixture was stirred with basic resin to bring the pH up to 9-10. Then the mixture was filtered and concentrated under reduced pressure to give the title compound (300 mg) as a yellow oil. LC-MS (ESI⁺) m/z 502.4 (M+H)⁺.

### (2S,4R)—N-(2-(azetidin-3-yloxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (Intermediate EJ)

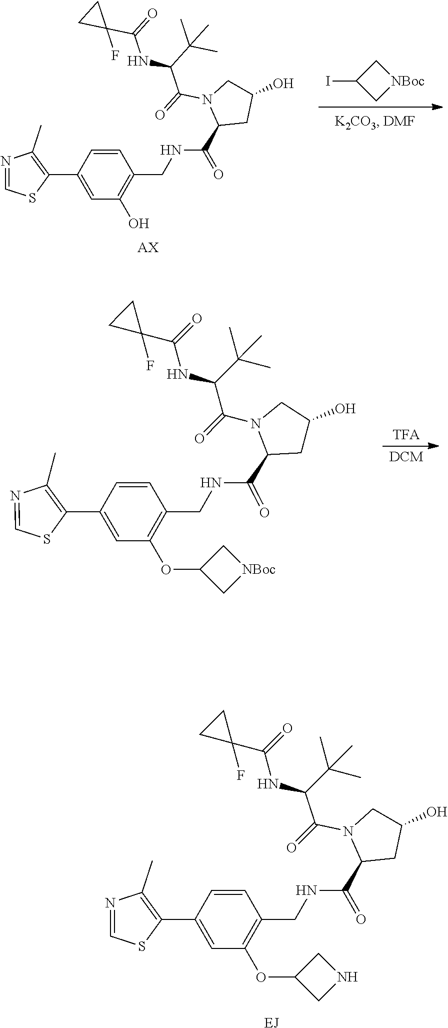

Step 1-Tert-butyl 3-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)azetidine-1-carboxylate. To a solution of (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (160 mg, 300 umol, Intermediate AX) and tert-butyl 3-iodoazetidine-1-carboxylate (119 mg, 420 umol) in DMF (1 mL) was added K₂CO₃ (83.0 mg, 600 umol). Then the mixture was stirred at 100° C. for 2 hours. On completion, the reaction mixture was diluted with H₂O (10 mL) and extracted with EA (30 mL×3). The combined organic layers were washed with brine (30 mL), dried over Na₂SO₄, filtered and concentrated under reduced pressure to give a residue. The residue was purified by column chromatography (SiO₂, Petroleum ether/Ethyl acetate=1/1 to 1/5) to give the title compound (100 mg, 46% yield) as a white solid. LC-MS (ESI⁺) m/z 688.2 (M+H)⁺.

Step 2-(2S,4R)—N-(2-(azetidin-3-yloxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide. To a solution of tert-butyl 3-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]azetidine-1-carboxylate (100 mg, 145 umol) in DCM (3 mL) was added TFA (0.5 mL), then the mixture was stirred at 25° C. for 1 hour. On completion, the mixture was stirred with basic resin until the pH=9-10. Then the mixture was filtered and concentrated under reduced pressure to give the title compound (60 mg) as a yellow oil. LC-MS (ESI⁺) m/z 588.2 (M+H)⁺.

### (S)-2-(8-(5-(1-(2-azaspiro[3.3]heptan-6-yl)piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate EK)

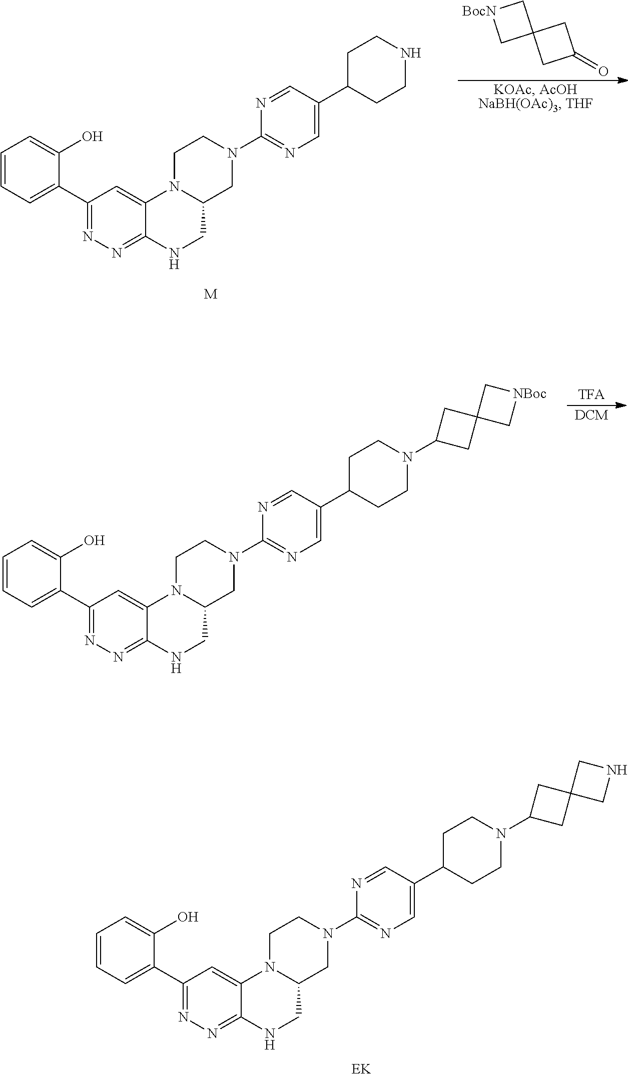

Step 1-(S)-tert-butyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptane-2-carboxylate. To a solution of (S)-2-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (300 mg, 675 umol, Intermediate M) in DMSO (2 mL) was added KOAc (198 mg, 2.02 mmol), THF (3 mL), NaBH(OAc)₃ (358 mg, 1.69 mmol), 4 Å molecular sieves (200 mg), tert-butyl 6-oxo-2-azaspiro[3.3]heptane-2-carboxylate (214 mg, 1.01 mmol) and HOAc (122 mg, 2.02 mmol, 116 uL). The mixture was then stirred at 0-25° C. for 3 hours. On the completion, the mixture was filtered by disposable needle filter. Then the crude product was purified by reversed-phase flash chromatography (0.1% HCl condition) to give the title compound (330 mg, 62% yield) as a yellow solid. ¹H NMR (400 MHz, DMSO-d6) δ=14.48-13.91 (m, 1H), 11.63-10.98 (m, 1H), 10.87-10.30 (m, 1H), 8.34 (s, 2H), 8.26-8.13 (m, 1H), 7.47 (d, J=7.6 Hz, 1H), 7.41 (t, J=7.2 Hz, 1H), 7.18 (s, 1H), 7.11 (br d, J=8.0 Hz, 1H), 6.99 (t, J=7.2 Hz, 1H), 4.72 (br d, J=12.0 Hz, 1H), 4.62 (s, 1H), 4.32-4.18 (m, 1H), 3.89 (s, 2H), 3.79 (s, 2H), 3.72 (d, J=8.8 Hz, 3H), 3.39-3.33 (m, 3H), 3.29 (d, J=10.2 Hz, 3H), 2.99-2.87 (m, 2H), 2.83-2.73 (m, 3H), 2.63-2.56 (m, 2H), 2.05-1.95 (m, 4H), 1.42-1.31 (m, 9H).

Step 2-(S)-2-(8-(5-(1-(2-azaspiro[3.3]heptan-6-yl)piperidin-4-yl)pyrimidin-2-yl)-6,6a,7,8,9,10-hexahydro-5H-pyrazino[1′,2′:4,5]pyrazino[2,3-c]pyridazin-2-yl)phenol. To a solution of (S)-tert-butyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptane-2-carboxylate (160 mg, 250 umol) in DCM (8 mL) was added TFA (924 mg, 8.10 mmol, 0.6 mL). The mixture was then stirred at 25° C. for 0.5 hour. On completion, the reaction mixture was concentrated to remove the solvent to give the title compound (160 mg) as a yellow oil. LC-MS (ESI⁺) m/z 540.1 (M+H)⁺.

### (S)-7-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-2-carboxylic acid (Intermediate EL)

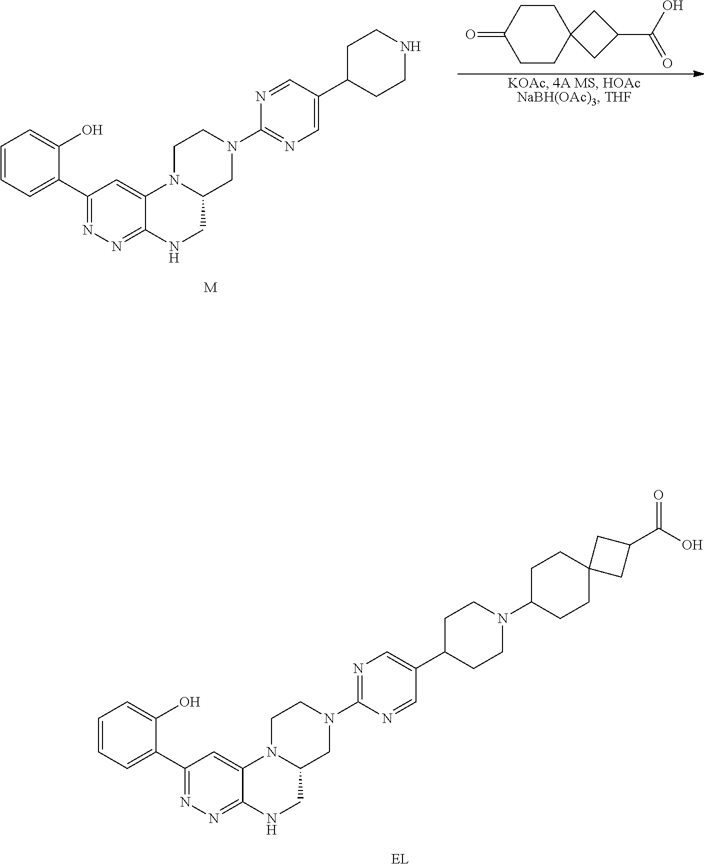

To a solution of (S)-2-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (240 mg, 499 umol, HCl, Intermediate M) in THF (2 mL) was added KOAc (147 mg, 1.50 mmol), NaBH(OAc)₃ (264 mg, 1.25 mmol), DMSO (1.2 mL), 7-oxospiro[3.5]nonane-2-carboxylic acid (136 mg, 748 umol), 4 Å molecular sieves (100 mg) and HOAc (90 mg, 1.50 mmol). The mixture was stirred at 25° C. for 3 hours. On the completion, the mixture was filtered by disposable needle filter. Then the crude product was purified by reversed-phase flash chromatography (0.1% HCl condition) to give the title compound (304 mg, 85% yield) as a white solid. LC-MS (ESI+) m/z 611.6 (M+H)⁺.

### (S)-2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid (Intermediate EM)

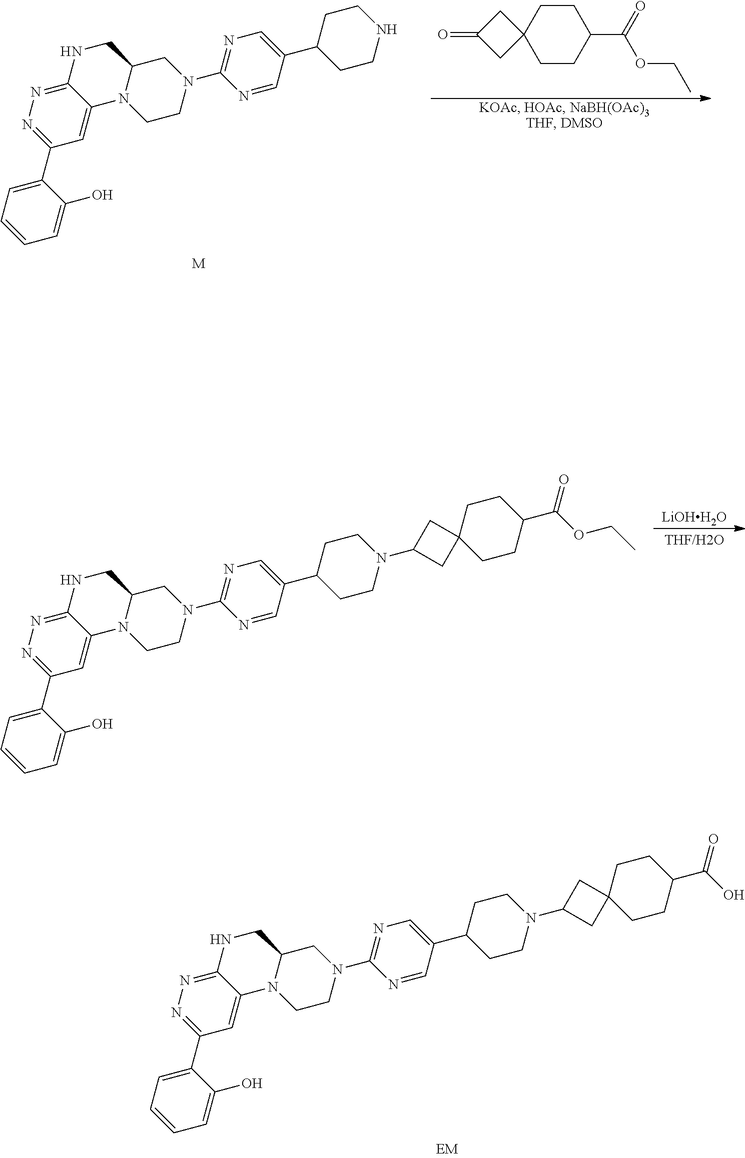

Step 1-(S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate. To a solution of 2-[(10S)-12-[5-(4-piperidyl)pyrimidin-2-yl]-1,5,6,8,12-pentazatricyclo[8.4.0.0^{2,7}]tetradeca-2,4,6-trien-4-yl]phenol (1.1 g, 2.29 mmol, Intermediate M) in THF (8 mL) and DMSO (4 mL) was added KOAc (673 mg, 6.86 mmol) and the reaction was stirred at 25° C. for 0.5 hour. Then ethyl 2-oxospiro[3.5]nonane-7-carboxylate (962 mg, 4.57 mmol) and HOAc (549 mg, 9.15 mmol) was added to the mixture and the mixture was stirred at 0° C. for 0.5 hour. Finally, NaBH(OAc)₃ (1.45 g, 6.86 mmol) was stirred at 0-25° C. for 3 hours. On completion, the reaction mixture was filtered and concentrated under reduced pressure to give a residue. The crude product was purified by reversed-phase HPLC (0.1% FA condition) to give the title compound (520 mg, 34% yield) as a yellow solid. ¹H NMR (400 MHz, DMSO-d₆) δ=8.34 (s, 2H), 8.21 (s, 1H), 7.94 (d, J=7.6 Hz, 1H), 7.40 (s, 1H), 7.31 (s, 1H), 7.21 (t, J=7.6 Hz, 1H), 6.91-6.77 (m, 2H), 4.72 (dd, J=4.4, 8.4 Hz, 2H), 4.19 (d, J=12.4 Hz, 1H), 4.03 (q, J=7.2 Hz, 2H), 3.61 (d, J=11.2 Hz, 1H), 3.31-3.23 (m, 1H), 3.18-3.08 (m, 1H), 3.03-2.89 (m, 3H), 2.85-2.68 (m, 2H), 2.45-2.38 (m, 1H), 2.26-2.17 (m, 1H), 1.99-1.79 (m, 4H), 1.75-1.56 (m, 9H), 1.55-1.31 (m, 6H), 1.16 (t, J=7.2 Hz, 3H). LC-MS (ESI⁺) m/z 639.4 (M+H)⁺.

Step 2-(S)-2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid. To a solution of ethyl 2-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]spiro[3.5]nonane-7-carboxylate (500 mg, 783 umol) in THF (2 mL) and MeOH (2 mL) was added LiOH.H₂O (2 M, 2 mL) and NaOH (2 M, 2 mL). The mixture was stirred at 25° C. for 12 hours. On completion, the reaction mixture was filtered and concentrated under reduced pressure to give the title compound (500 mg) as a white solid. LC-MS (ESI⁺) m/z 611.2 (M+H)⁺.

### 2-(3-(6-(4-(2-((R)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptan-2-yl)isoxazol-5-yl)-3-methylbutanoic acid (Intermediate EN)

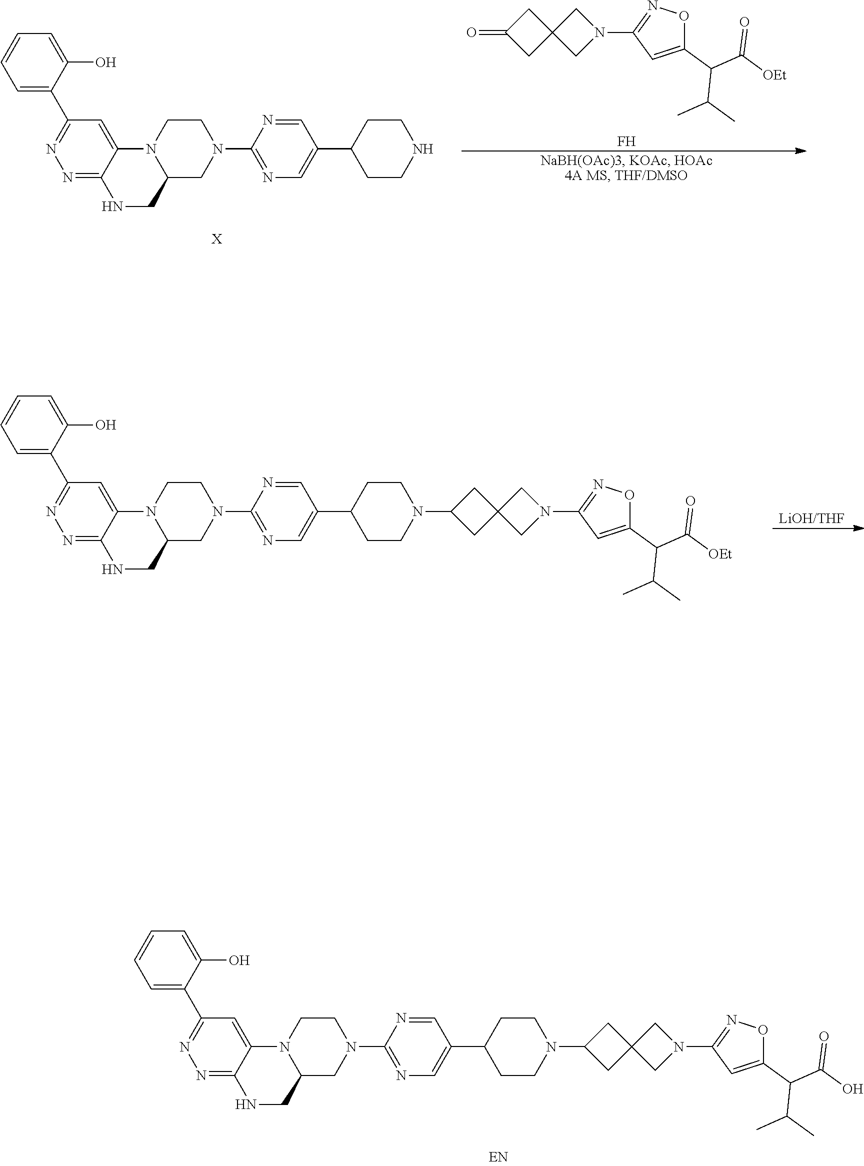

Step 1-Ethyl 2-(3-(6-(4-(2-((R)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptan-2-yl)isoxazol-5-yl)-3-methylbutanoate. To a solution of 2-[(10R)-12-[5-(4-piperidyl)pyrimidin-2-yl]-1,5,6,8,12-pentazatricyclo[8.4.0.0^{2,7}]tetradeca-2,4,6-trien-4-yl]phenol (500 mg, 1.12 mmol, Intermediate X) in THF (4 mL) and DMSO (1 mL) was added KOAC (329 mg, 3.36 mmol), HOAc (201 mg, 3.36 mmol), 4 Å molecular sieves (500 mg) and ethyl 3-methyl-2-[3-(6-oxo-2-azaspiro[3.3]heptan-2-yl)isoxazol-5-yl]butanoate (343 mg, 1.12 mmol, Intermediate FH). The mixture was then stirred at 0° C. for 1 hour. Then NaBH(OAc)₃ (712 mg, 3.36 mmol) was added into the mixture and the mixture was stirred at 40° C. for 4 hours. On completion, the reaction mixture was filtrated to give a filtrate. The filtrate was purified by prep-HPLC (FA condition) to give the title compound (240 mg, 28% yield) as a white solid. LC-MS (ESI⁺) m/z 735.7 (M+H)⁺.

Step 2-2-(3-(6-(4-(2-((R)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptan-2-yl)isoxazol-5-yl)-3-methylbutanoic acid. To a solution of ethyl 2-[3-[6-[4-[2-[(1OR)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]-2-azaspiro[3.3]heptan-2-yl]isoxazol-5-yl]-3-methyl-butanoate (240 mg, 326 umol) in THF (2 mL) and H₂O (0.5 mL) was added LiOH—H₂O (41.1 mg, 979 umol). The mixture was then stirred at 30° C. for 3 hours. On completion, the reaction mixture was filtrated to give a filtrate. The filtrate was concentrate under reduced pressure to give the title compound (240 mg) as a brown solid. LC-MS (ESI⁺) m z 707.7 (M+H)⁺.

### (2S,4R)—N-(4-cyanobenzyl)-4-hydroxypyrrolidine-2-carboxamide (Intermediate EO)

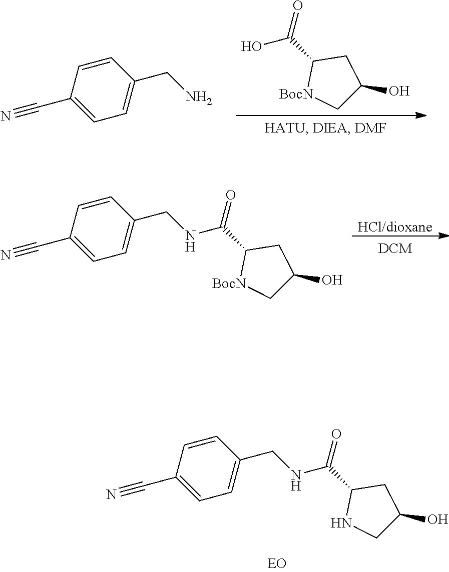

Step 1-(2S,4R)-tert-butyl 2-((4-cyanobenzyl)carbamoyl)-4-hydroxypyrrolidine-1-carboxylate. HATU (51.81 g, 136.25 mmol) was added to a solution of (2S,4R)-1-tert-butoxycarbonyl-4-hydroxy-pyrrolidine-2-carboxylic acid (30 g, 129.76 mmol, CAS #13726-69-7) in DMF (180 mL), then the reaction solution was cooled to 0° C. Next, DIPEA (41.93 g, 324 mmol) was added, followed by 4-(aminomethyl)benzonitrile (17.15 g, 130, CAS #10406-25-4) in DMF (40 mL) slowly into the solution. Then the reaction solution was warmed to 25° C. within 30 mins, and stirred for another 18 h at 25° C. On completion, the reaction solution was quenched with aqueous NH₄Cl solution (150 mL), then was extracted by EA (3×300 ml). The combined organic layers were washed by saturated aqueous NH₄Cl solution (150 mL) and brine (50 mL), and then dried over Na₂SO₄. The concentrated residue was purified by column chromatography (SiO₂, Ethyl acetate: Methanol=I/O to 20/1) to give the title compound (39.5 g, 88% yield) as a yellow solid. LC-MS (ESI⁺) m/z 246.3 (M-99)⁺.

Step 2-(2S,4R)—N-(4-cyanobenzyl)-4-hydroxypyrrolidine-2-carboxamide. To a solution of tert-butyl (2S,4R)-2-[(4-cyanophenyl)methylcarbamoyl]-4-hydroxy-pyrrolidine-1-carboxylate (55 g, 159 mmol) in DCM (50 mL) was added HCl/dioxane (4 M, 188.57 mL). The mixture was stirred at 0-25° C. for 2 hours. On completion, the suspension reaction mixture was filtered and the filter cake was washed by DCM to give the title compound (27 g, 56% yield) as a white solid. LC-MS (ESI⁺) m/z 246.0 (M+H)⁺. 2-(3-(6-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptan-2-yl)isoxazol-5-yl)-3-methylbutanoic acid (Intermediate EP)

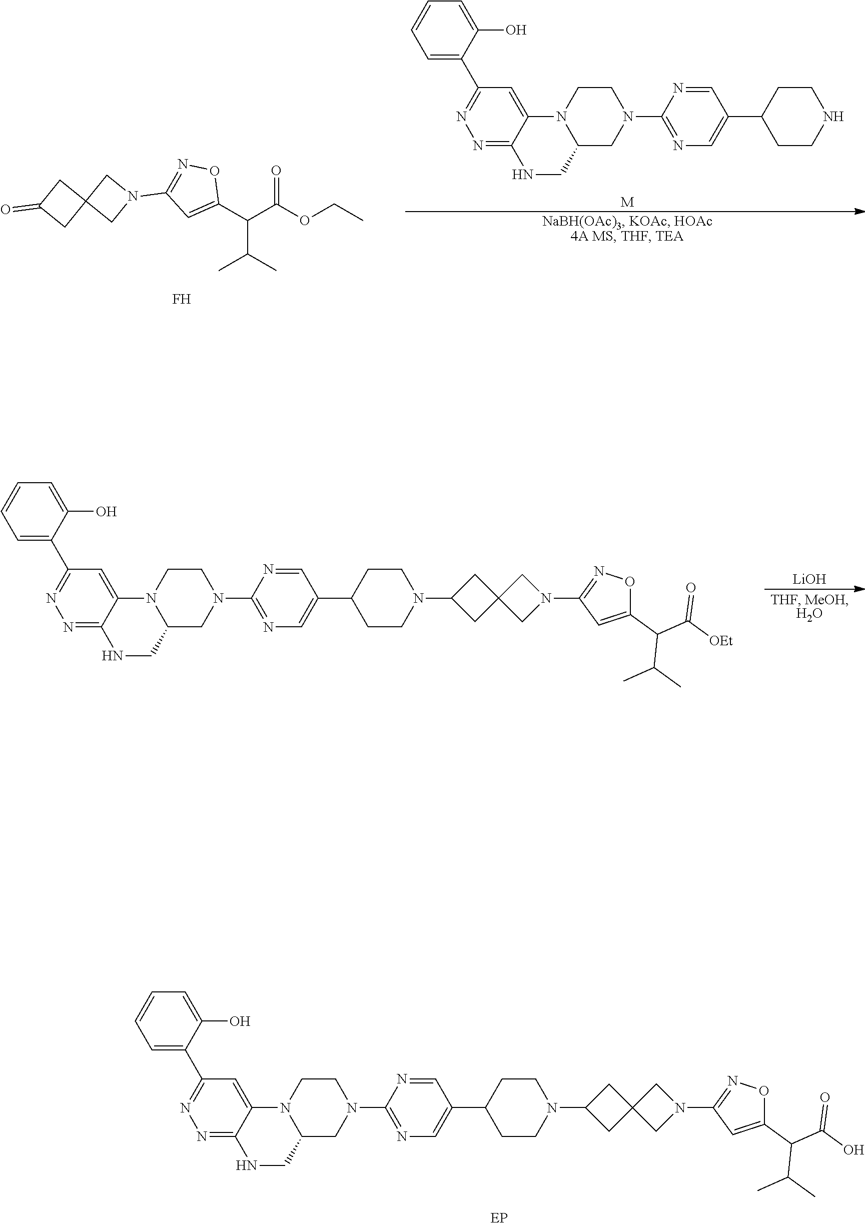

Step 1-Ethyl 2-(3-(6-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptan-2-yl)isoxazol-5-yl)-3-methylbutanoate. To a solution of (S)-2-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (140 mg, 291 umol, HCl, Intermediate M) in THF (4 mL) was added TEA (29.5 mg, 291 umol, 40.5 uL), ethyl 3-methyl-2-(3-(6-oxo-2-azaspiro[3.3]heptan-2-yl)isoxazol-5-yl)butanoate (89.2 mg, 291 umol, Intermediate FH), 4 Å molecular sieves (50 mg), AcOH (1.75 mg, 29.1 umol, 1.66 uL), and KOAc (57.1 mg, 582 umol,) at 0° C. The mixture was stirred for 2 hours, then NaBH(OAc)₃ (154 mg, 728 umol) was added and the mixture was stirred at 40° C. for 2 hours. On completion, the reaction mixture was filtered and concentrated under reduced pressure to give a residue. The crude product was purified by reversed-phase HPLC (0.1% FA condition) to give the title compound (80 mg, 37% yield) as white solid. LC-MS (ESI⁺) m/z 735.3 (M+H)⁺.

Step 2-2-(3-(6-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptan-2-yl)isoxazol-5-yl)-3-methylbutanoic acid. To a solution of ethyl 2-(3-(6-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)-2-azaspiro[3.3]heptan-2-yl)isoxazol-5-yl)-3-methylbutanoate (80 mg, 109 umol) in THF (1 mL), MeOH (1 mL) and H₂O (0.5 mL) was added LiOH (10.4 mg, 435 umol). Then the mixture was stirred at 30° C. for 1 hour. On completion, the reaction mixture was filtered and concentrated under reduced pressure to give a residue. The crude product was purified by reversed-phase HPLC (0.1% FA condition) to give the title compound (40 mg, 47% yield) as yellow solid. LC-MS (ESI+) m/z 707.4 (M+H)⁺.

### (2S,4R)—N-[(4-chlorophenyl)methyl]-4-hydroxy-pyrrolidine-2-carboxamide (Intermediate EQ)

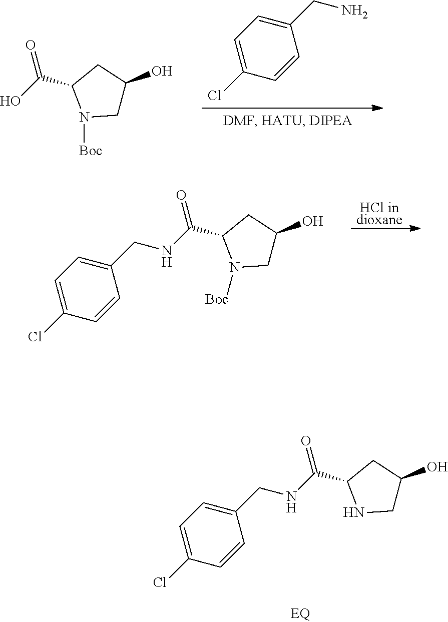

Step 1-(2S,4R)-tert-butyl 2-((4-chlorobenzyl)carbamoyl)-4-hydroxypyrrolidine-1-carboxylate. To a solution of (2S,4R)-1-tert-butoxycarbonyl-4-hydroxy-pyrrolidine-2-carboxylic acid (10 g, 43 mmol, CAS #13726-69-7) and (4-chlorophenyl)methanamine (6.7 g, 48 mmol, 5.8 mL, CAS #104-86-9) in DMF (200 mL) was added HATU (20 g, 52 mmol) and DIPEA (6.7 g, 52 mmol, 9.0 mL. Then the mixture stirred at 20° C. for 2 hrs. On completion, the reaction mixture was quenched with water (100 mL), and then diluted with EA (100 mL) and extracted with EA (150 mL×3). The combined organic layers were washed with sat. brine (200 mL), dried over Na₂SO₄, filtered and concentrated under reduced pressure to give the title compound (15 g, 42 mmol, 98% yield) as a yellow oil. LC-MS (ESI, m/z): [M −99]⁺=255.0.

Step 2-(2S,4R)—N-(4-chlorobenzyl)-4-hydroxypyrrolidine-2-carboxamide. A solution of tert-butyl (2S,4R)-2-[(4-chlorophenyl)methylcarbamoyl]-4-hydroxy-pyrrolidine-1-carboxylate (15 g, 42 mmol) in HCl/dioxane (100 mL) was stirred at 25° C. for 0.5 hrs. On completion, the mixture was filtered and the result residue was concentrated under reduced pressure to give a residue. The crude product was triturated with DCM (150 mL) at 25° C. for 3 hrs. The mixture was then filtered and the resulting residue was concentrated under reduced pressure to give the title compound (12 g, 91% yield, HCl salt). LC-MS (ESI, m/z): [M+1]⁺=254.9; ¹H NMR (400 MHz, D2O) δ (ppm)=7.35 (br d, J=8.3 Hz, 2H), 7.24 (br d, J=8.1 Hz, 2H), 4.70-4.55 (m, 2H), 4.46-4.28 (m, 2H), 3.51-3.34 (m, 2H), 2.48 (br dd, J=7.6, 13.8 Hz, 1H), 2.19-2.07 (m, 1H).

### (S)-2-(8-(4-(piperazin-1-yl)pyrimidin-2-yl)-6,6a,7,8,9,10-hexahydro-5H-pyrazino[1′,2′:4,5]pyrazino[2,3-c]pyridazin-2-yl)phenol (Intermediate ER)

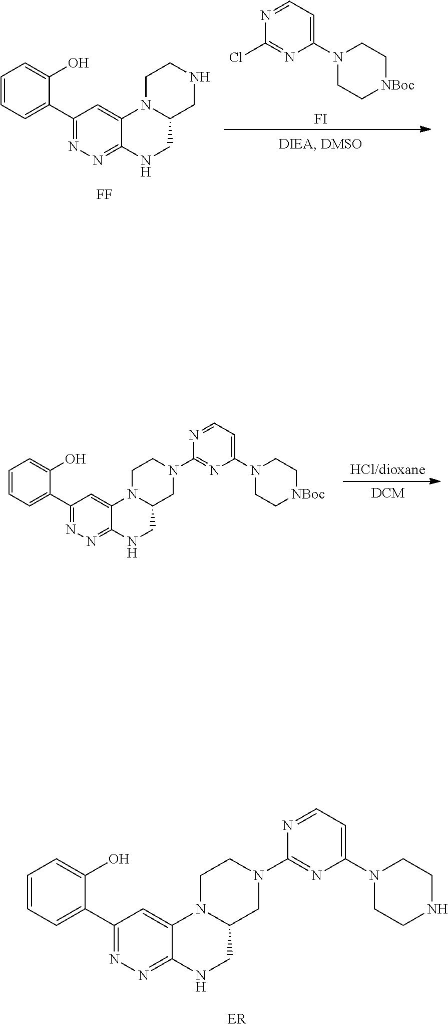

Step 1 (S)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperazine-1-carboxylate. To a solution of 2-[(1OR)-1,5,6,8,12-pentazatricyclo[8.4.0.02,7]tetradeca-2,4,6-trien-4-yl]phenol (600 mg, 2.12 mmol, Intermediate FF) in DMSO (15 mL) was added DIEA (1.51 g, 11.7 mmol) and tert-butyl 4-(2-chloropyrimidin-4-yl)piperazine-1-carboxylate (823 mg, 2.75 mmol, Intermediate FI) at 25° C. Then the reaction was stirred at 90° C. for 12 hours. On completion, the reaction mixture was triturated with H₂O at 25° C. for 10 minutes, filtered and concentrated in vacuo to get the crude residue. The residue was purified by column chromatography (SiO₂, Petroleum ether/Ethyl acetate=1/1 to DCM: MeOH=10/1) to give the title compound (580 mg, 47% yield) as brown solid. LC-MS (ESI*) m/z 552.5. (M+H)⁺.

Step 2-(S)-2-(8-(4-(piperazin-1-yl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl 4-[2-[(10S)-4-(2-hydroxyphenyl)-1,5,6,8,12-pentazatricyclo[8.4.0.0^{2,7}]tetradeca-2,4,6-trien-12-yl]pyrimidin-4-yl]piperazine-1-carboxylate (280 mg, 513 umol) was added in DCM (3 mL) and HCl/dioxane (1 mL). The mixture was then stirred at 25° C. for 1 hour. On completion, the reaction mixture was filtered and concentrated under reduced pressure to give the title compound (230 mg) as a yellow solid. LC-MS (ESI, m/z): [M+1]⁺=446.2.

### (S)-2-(8-(4-(4-(2-azaspiro[3.3]heptan-6-yl)piperazin-1-yl)pyrimidin-2-yl)-6,6a,7,8,9,10-hexahydro-5H-pyrazino[1′,2′:4,5]pyrazino[2,3-c]pyridazin-2-yl)phenol (Intermediate ES)

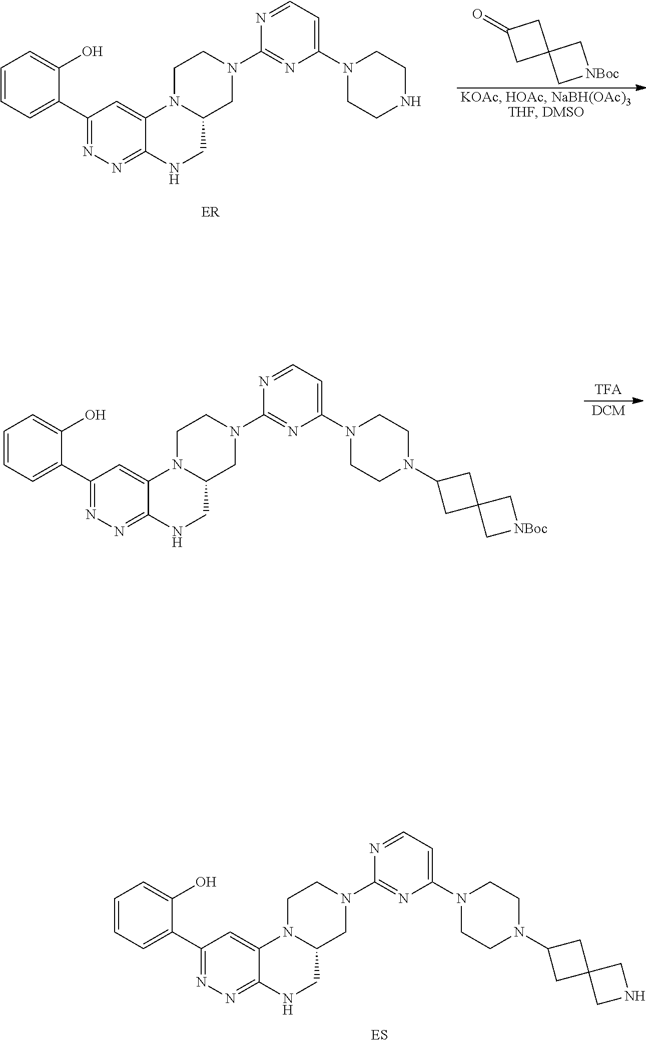

Step 1-(S)-tert-butyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-4-yl)piperazin-1-yl)-2-azaspiro[3.3]heptane-2-carboxylate. To a solution of 2-[(10S)-12-(4-piperazin-1-ylpyrimidin-2-yl)-1,5,6,8,12-pentazatricyclo[8.4.0.0^{2,7}]tetradeca-2,4,6-trien-4-yl]phenol (200 mg, 449 umol, Intermediate ER) and KOAc (132 mg, 1.35 mmol) in THF (2 mL) and DMSO (1 mL) was stirred at 25° C. for 30 minutes. Then tert-butyl 6-oxo-2-azaspiro[3.3]heptane-2-carboxylate (114 mg, 539 umol) and HOAc (108 mg, 1.80 mmol) was added to the mixture and the mixture was stirred at 25° C. for 30 minutes. Finally, NaBH(OAc)₃ (285 mg, 1.35 mmol) was added to the mixture which was then stirred at 0-25° C. for 12 hours. On completion, the reaction mixture was filtered and concentrated under reduced pressure to give a residue. The crude product was purified by reversed-phase HPLC (0.1% FA condition) to give the title compound (120 mg, 39% yield) as a yellow solid. LC-MS (ESI⁺) m/z 641.4 (M+H)⁺.

Step 2-(S)-2-(8-(4-(4-(2-azaspiro[3.3]heptan-6-yl)piperazin-1-yl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl 6-[4-[2-[(10S)-4-(2-hydroxyphenyl)-1,5,6,8,12-pentazatricyclo[8.4.0.0^{2,7}]tetradeca-2,4,6-trien-12-yl]pyrimidin-4-yl]piperazin-1-yl]-2-azaspiro[3.3]heptane-2-carboxylate (130 mg, 203 umol) was added in DCM (2 mL) and TFA (0.2 mL). Then the mixture was stirred at 25° C. for 1 hour. On completion, the reaction mixture was filtered and concentrated under reduced pressure to give the title compound (60 mg) as a yellow solid. LC-MS (ESI⁺) m/z 541.3 (M+H)⁺.

### (2S,4R)-1-((S)-2-amino-3,3-dimethylbutanoyl)-4-hydroxy-N—((S)-1-(4-(1-methyl-1H-pyrazol-5-yl)phenyl)ethyl)pyrrolidine-2-carboxamide (Intermediate ET)

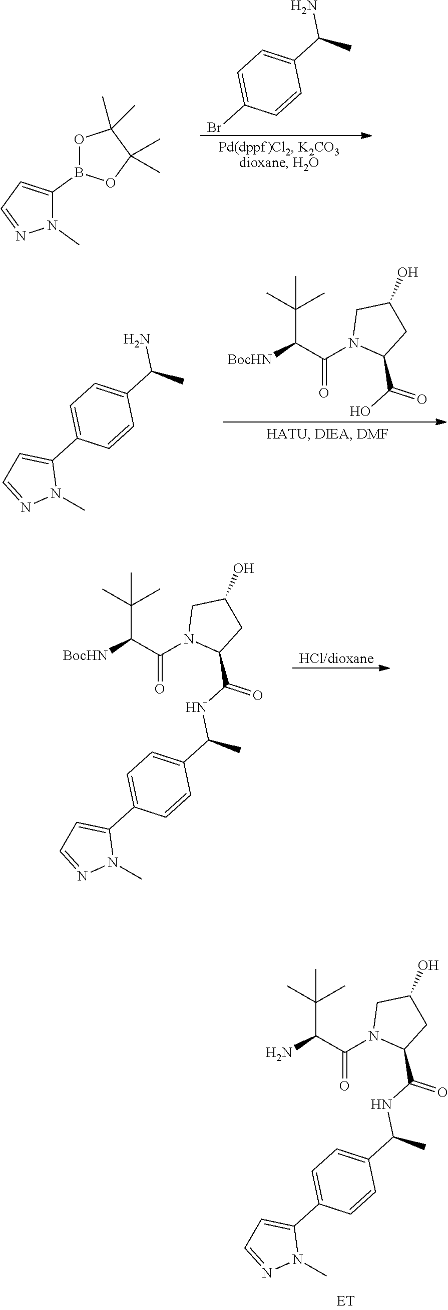

Step 1-(S)-1-(4-(1-methyl-1H-pyrazol-5-yl)phenyl)ethanamine. To a solution of 1-methyl-5-(4,4,5,5-tetramethyl-1,3,2-dioxaborolan-2-yl)pyrazole (1.56 g, 7.50 mmol, CAS 847818-74-0), and (1S)-1-(4-bromophenyl)ethanamine (1 g, 5.0 mmol, CAS 27298-97-1) in dioxane (30 mL) and H₂O (6 mL) was added Pd(dppf)Cl₂ (365 mg, 499 umol) and K₂CO₃ (1.73 g, 12.5 mmol). The mixture was degassed and purged with N₂ three times, and then the mixture was stirred at 100° C. for 4 hours. On completion, the reaction mixture was diluted with H₂O (30 mL) and extracted with EA (300 mL×3). The combined organic layers were washed with brine (50 mL×2), dried over Na₂SO₄, filtered and concentrated under reduced pressure to give a residue. The residue was purified by prep-HPLC (FA condition) to give the title compound (1.1 g, 89% yield, FA) as white solid. LC-MS (ESI⁺) m/z 202.0 (M+H)⁺.

Step 2-Tert-butyl ((S)-1-((2S,4R)-4-hydroxy-2-(((S)-1-(4-(1-methyl-1H-pyrazol-5-yl)phenyl)ethyl)carbamoyl)pyrrolidin-1-yl)-3,3-dimethyl-1-oxobutan-2-yl)carbamate. To a solution of (1S)-1-[4-(2-methylpyrazol-3-yl)phenyl]ethanamine (1.1 g, 4.45 mmol, FA), (2S,4R)-1-[(2S)-2-(tert-butoxycarbonylamino)-3,3-dimethyl-butanoyl]-4-hydroxy-pyrrolidine-2-carboxylic acid (1.99 g, 5.78 mmol, CAS #630421-46-4) in DMF (30 mL) was added HATU (2.54 g, 6.67 mmol) and DIEA (2.30 g, 17.8 mmol). The mixture was stirred at 25° C. for 12 hours. On completion, the reaction mixture was diluted with H₂O (30 mL) and extracted with EA (200 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=1/1 to 1/10). The residue was then repurified by prep-HPLC (FA condition) to give the title compound (1.1 g, 43% yield, FA) as a yellow solid. LC-MS (ESI⁺) m/z 528.3 (M+H)⁺.

Step 3-(2S,4R)-1-((S)-2-amino-3,3-dimethylbutanoyl)-4-hydroxy-N—((S)-1-(4-(1-methyl-1H-pyrazol-5-yl)phenyl)ethyl)pyrrolidine-2-carboxamide. To a solution of tert-butyl N-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2,2-dimethyl-propyl]carbamate (1.1 g, 2.08 mmol) in DCM (20 mL) was added HCl/dioxane (4 M, 4.35 mL). The mixture was then stirred at 25° C. for 2 hours. On completion, the reaction mixture was filtered and concentrated under reduced pressure to give the title compound (1 g, HCl) as a brown solid. LC-MS (ESI⁺) m/z 428.0 (M+H)⁺.

(4-Ethynylphenyl)methanamine (Intermediate EU)

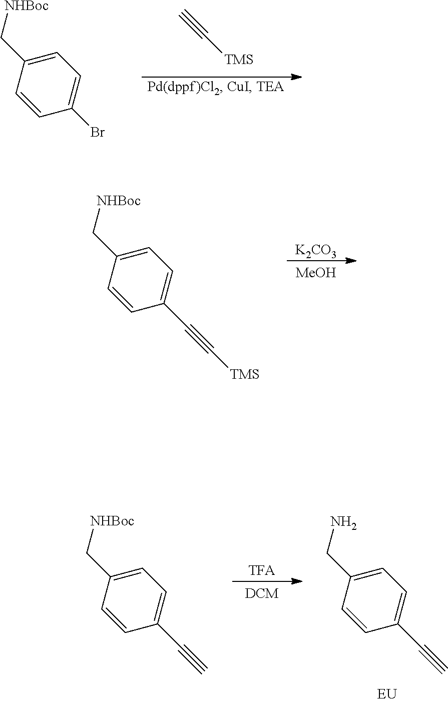

Step 1-Tert-butyl 4-((trimethylsilyl)ethynyl)benzylcarbamate. A mixture of tert-butyl 4-bromobenzylcarbamate (10 g, 34.9 mmol), ethynyltrimethylsilane (10.3 g, 105 mmol), CuI (666 mg, 3.49 mmol), Pd(dppf)Cl₂ (1.28 g, 1.75 mmol) in TEA (100 mL) was degassed and purged with N₂ three times. Then the mixture was stirred at 80° C. for 12 hours under N₂ atmosphere. On completion, the reaction mixture was filtered and concentrated under reduced pressure to give a residue. The residue was purified by prep-HPLC (0.1% FA) to give the title compound (8.4 g, 67% yield) as a white solid. LC-MS (ESI+) m/z 248.1 (M-55)⁺.

Step 2-Tert-butyl 4-ethynylbenzylcarbamate. To a solution of tert-butyl 4-((trimethylsilyl)ethynyl)benzylcarbamate (4 g, 13.2 mmol) in MeOH (50 mL) was added K₂CO₃ (3.64 g, 26.4 mmol). Then the mixture was stirred at 25° C. for 2 hours. On completion, the reaction mixture was diluted with H₂O (50 mL) and extracted with EA (100 mL×3). The combined organic layers were dried over Na₂SO₄, filtered and concentrated under reduced pressure to give the title compound (3 g) as a yellow solid. LC-MS (ESI⁺) m/z 176.3 (M-55)⁺.

Step 3-(4-Ethynylphenyl)methanamine. To a solution of tert-butyl 4-ethynylbenzylcarbamate (3 g, 13.0 mmol) in DCM (20 mL) was added TFA (5.92 g, 51.9 mmol). The mixture was then stirred at 25° C. for 0.5 hour. On completion, the reaction mixture was concentrated under reduced pressure to give the title compound as a yellow solid (1.24 g). ¹H NMR (400 MHz, DMSO-d₆) δ=8.36 (s, 3H), 7.56-7.51 (m, 2H), 7.50-7.45 (m, 2H), 4.07 (s, 2H), 3.95-3.54 (m, 1H).

### (2S,4R)-1-((S)-2-((tert-butoxycarbonyl)amino)-3,3-dimethylbutanoyl)-4-hydroxypyrrolidine-2-carboxylic acid (Intermediate EV)

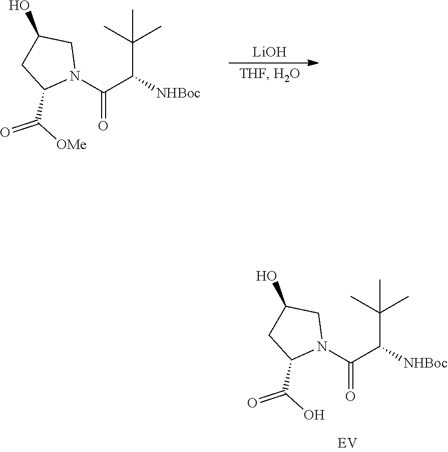

To a solution of (2S,4R)-methyl 1-((S)-2-((tert-butoxycarbonyl)amino)-3,3-dimethylbutanoyl)-4-hydroxypyrrolidine-2-carboxylate (30 g, 83.7 mmol, CAS #630421-45-3) in THF (150 mL) and H₂O (150 mL) was added LiOH (6.01 g, 251 mmol). The mixture was then stirred at 25° C. for 2 hours. On completion, the reaction mixture was quenched with HCl(1 M) until the pH was 3, and then the mixture was extracted with EA (200 mL×3). The combined organic layers were dried over Na₂SO₄, filtered and concentrated under reduced pressure to give the title compound (23.4 g) as a yellow oil. LC-MS (ESI⁺) m/z 289.1 (M-55)⁺.

### (2S,4R)-1-((S)-2-amino-3,3-dimethylbutanoyl)-N-(4-ethynylbenzyl)-4-hydroxypyrrolidine-2-carboxamide (Intermediate EW)

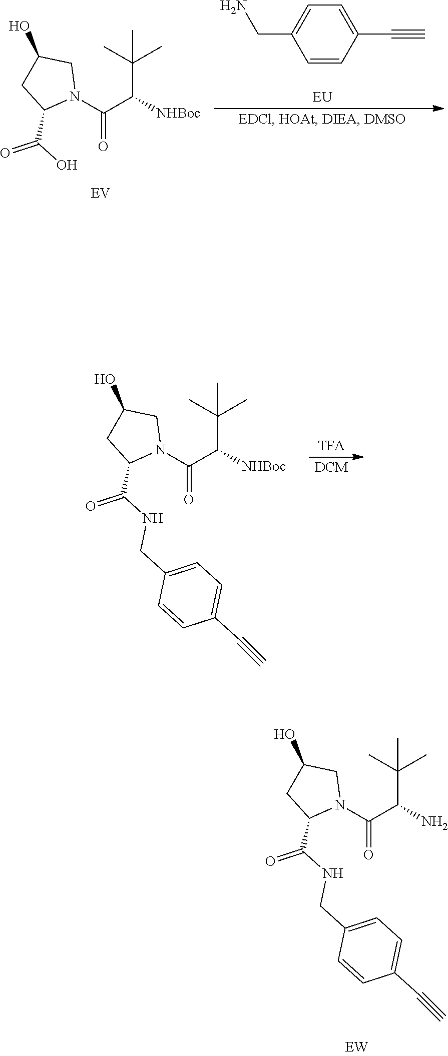

Step 1-Tert-butyl ((S)-1-((2S,4R)-2-((4-ethynylbenzyl)carbamoyl)-4-hydroxypyrrolidin-1-yl)-3,3-dimethyl-1-oxobutan-2-yl)carbamate. To a solution of (2S,4R)-1-((S)-2-((tert-butoxycarbonyl)amino)-3,3-dimethylbutanoyl)-4-hydroxypyrrolidine-2-carboxylic acid (3 g, 8.71 mmol, Intermediate EV) in DMSO (20 mL) was added EDCI (2.50 g, 13.1 mmol), HOAt (1.78 g, 13.1 mmol), DIEA (5.63 g, 43.6 mmol) and (4-ethynylphenyl)methanamine (1.14 g, 4.66 mmol, Intermediate EU). The mixture was then stirred at 25° C. for 2 hours. On completion, the reaction mixture was filtered and concentrated under reduced pressure to give a residue. The residue was purified by prep-HPLC (0.1% FA) to give the title compound (1.89 g, 43% yield) as a yellow solid. LC-MS (ESI+) m/z 458.1 (M+H)⁺.

Step 2-(2S,4R)-1-((S)-2-amino-3,3-dimethylbutanoyl)-N-(4-ethynylbenzyl)-4-hydroxypyrrolidine-2-carboxamide. To a solution of tert-butyl ((S)-1-((2S,4R)-2-((4-ethynylbenzyl)carbamoyl)-4-hydroxypyrrolidin-1-yl)-3,3-dimethyl-1-oxobutan-2-yl)carbamate (1.89 g, 4.13 mmol) in DCM (20 mL) was added TFA (2.83 g, 24.8 mmol). The mixture was then stirred at 25° C. for 2 hours. On completion, the reaction mixture was filtered and concentrated under reduced pressure to give a residue. The residue was purified by prep-HPLC (column: Phenomenex Luna C18 150*25 mm*10 um; mobile phase: [water(FA)-ACN];B %: 12%-42%,11 min) to give a mixture. Then the mixture was isolated by prep-HPLC(column: Phenomenex C18 150*25 mm*10 um; mobile phase: [water(10 Mm NH₃H₂O)-ACN]; B %: 20%-50%, 15 min) to give the title compound (450 mg, 27% yield) as a white solid. LC-MS (ESI+) m/z 358.2 (M+H)⁺.

### Phenyl ((S)-1-((2S,4R)-2-((4-ethynylbenzyl)carbamoyl)-4-hydroxypyrrolidin-1-yl)-3,3-dimethyl-1-oxobutan-2-yl)carbamate (Intermediate EX)

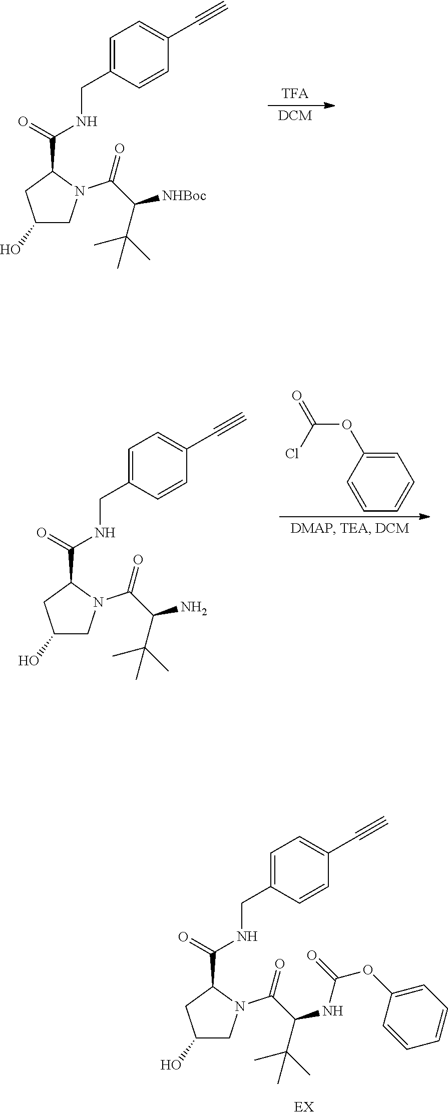 [SMILES: C#Cc1ccc(CNC(=O)[C@@H]2C[C@@H](O)CN2C(=O)[C@@H](NC(=O)OC(C)(C)C)C(C)(C)C)cc1 ; C#Cc1ccc(CNC(=O)[C@@H]2C[C@@H](O)CN2C(=O)[C@@H](N)C(C)(C)C)cc1 ; O=C(Cl)Oc1ccccc1 ; C#Cc1ccc(CNC(=O)[C@@H]2C[C@@H](O)CN2C(=O)[C@@H](NC(=O)Oc2ccccc2)C(C)(C)C)cc1]

Step 1-(2S,4R)-1-((S)-2-amino-3,3-dimethylbutanoyl)-N-(4-ethynylbenzyl)-4-hydroxypyrrolidine-2-carboxamide. To a mixture of tert-butyl ((S)-1-((2S,4R)-2-((4-ethynylbenzyl)carbamoyl)-4-hydroxypyrrolidin-1-yl)-3,3-dimethyl-1-oxobutan-2-yl)carbamate (3 g, 6.56 mmol, synthesized via Step 1 of Intermediate EW) in DCM (30 mL) was added TFA (4 M, 6 mL). The mixture was then stirred at 25° C. for 3 hours. On completion, the reaction mixture was concentrated in vacuo to give a residue. The crude product was purified by prep-HPLC (0.1% FA condition) to give the title compound (800 mg, 27% yield, FA) as a yellow solid. LC-MS (ESI+) m/z 358.1 (M+H)⁺.

Step 2-phenyl ((S)-1-((2S,4R)-2-((4-ethynylbenzyl)carbamoyl)-4-hydroxypyrrolidin-1-yl)-3,3-dimethyl-1-oxobutan-2-yl)carbamate. To a solution of (2S,4R)-1-((S)-2-amino-3,3-dimethylbutanoyl)-N-(4-ethynylbenzyl)-4-hydroxypyrrolidine-2-carboxamide (400 mg, 895.24 umol) in DCM (8 mL) was added TEA (271.77 mg, 2.69 mmol). Next, phenyl carbonochloridate (168.20 mg, 1.07 mmol) was added at 0° C., then the mixture was stirred at 25° C. for 1 hour. On completion, the reaction mixture was concentrated in vacuo to give a residue. The residue was purified by column chromatography (SiO₂, Petroleum ether/Ethyl acetate=6/1) to give the title compound (200 mg, 31% yield) as a white solid. LC-MS (ESI+) m/z 478.3 (M+H)⁺.

### (S)-1-(4-ethynylphenyl)ethanamine (Intermediate EY)

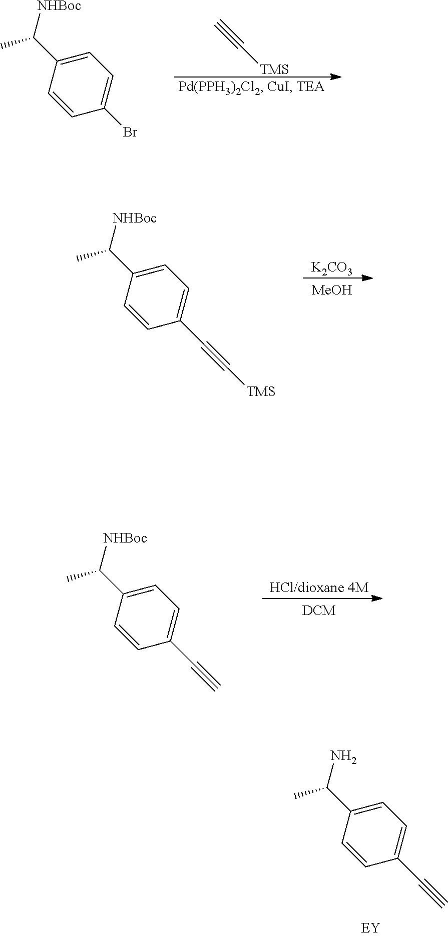

Step 1-(S)-tert-butyl (1-(4-((trimethylsilyl)ethynyl)phenyl)ethyl)carbamate. A mixture of tert-butyl N-[(1S)-1-(4-bromophenyl)ethyl]carbamate (2 g, 6.66 mmol, CAS #847728-89-6), ethynyl(trimethyl)silane (5.23 g, 53.3 mmol, 7.38 mL), Pd(PPh₃)₂Cl₂ (468 mg, 666 umol), CuI (254 mg, 1.33 mmol) in TEA (20 mL) was degassed and purged with N₂ three times. Then the mixture was stirred at 80° C. for 24 hours under N₂ atmosphere. On completion, the reaction mixture was diluted with H₂O (50 mL) and extracted with EA (60 mL×3). The combined organic layers were dried over Na₂SO₄, filtered and concentrated under reduced pressure to give a residue. The residue was purified by column chromatography (SiO₂, Petroleum ether/Ethyl acetate=I/O to 10/1) to give the title compound (1.80 g, 72% yield) as a yellow oil. LC-MS (ESI⁺) m/z 340.1 (M+H)⁺.

Step 2-(S)-tert-butyl (1-(4-ethynylphenyl)ethyl)carbamate. To a solution of tert-butyl N-[(1S)-1-[4-(2-trimethylsilylethynyl)phenyl]ethyl]carbamate (800 mg, 2.52 mmol) in MeOH (8 mL) was added K₂CO₃ (696 mg, 5.04 mmol). Then the mixture was stirred at 25° C. for 3.5 hrs. On completion, the reaction mixture was diluted with H₂O (20 mL) and extracted with EA (25 mL×4). The combined organic layers were dried over Na₂SO₄, filtered and concentrated under reduced pressure to give the title compound (600 mg) as a yellow solid. LC-MS (ESI⁺) m/z 268.2 (M+Na)⁺.

Step 3-(S)-1-(4-ethynylphenyl)ethanamine. To a solution of tert-butyl N-[(1S)-1-(4-ethynylphenyl)ethyl]carbamate (600 mg, 2.45 mmol) in DCM (12 mL) was added HCl/dioxane (4 M, 3 mL). The mixture was then stirred at 25° C. for 1 hr. On completion, the reaction mixture was concentrated under reduced pressure to give the title compound as a yellow solid (492 mg). LC-MS (ESI⁺) m/z 340.1 (M-16)⁺.

### (2S,4R)-1-((S)-2-amino-3,3-dimethylbutanoyl)-N—((S)-1-(4-ethynylphenyl)ethyl)-4-hydroxypyrrolidine-2-carboxamide (Intermediate EZ)

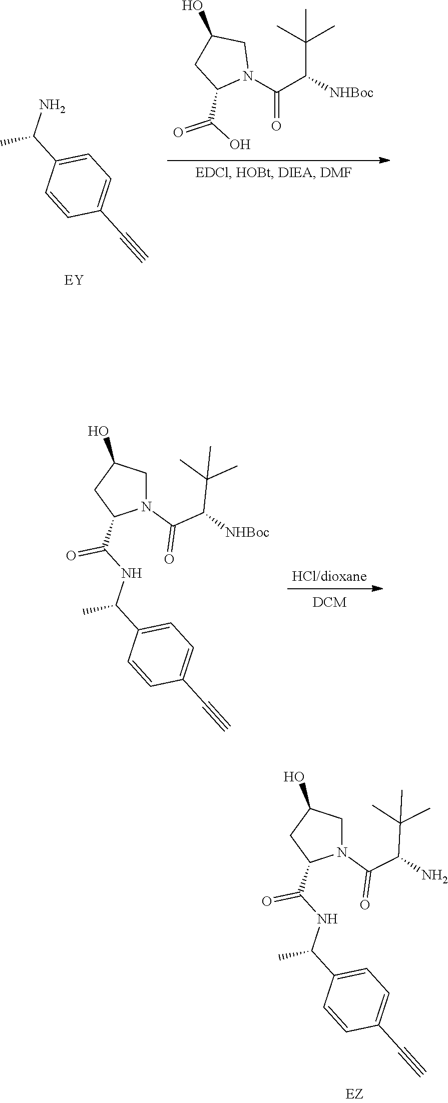

Step 1-Tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate. To a solution of (2S,4R)-1-[(2S)-2-(tert-butoxycarbonylamino)-3,3-dimethyl-butanoyl]-4-hydroxy-pyrrolidine-2-carboxylic acid (938 mg, 2.72 mmol, CAS #630421-46-4) in DMF (10 mL) was added EDCI (712 mg, 3.72 mmol) and HOBt (502 mg, 3.72 mmol) and the mixture was stirred at 0° C. for 0.5 hr. Then (1S)-1-(4-ethynylphenyl)ethanamine (450 mg, 2.48 mmol, HCl, Intermediate EY) and DIEA (3.20 g, 24.8 mmol) was added into the reaction mixture and the resulting mixture was stirred at 25° C. for 1 hour. On completion, the reaction mixture was diluted with H₂O (40 mL) and extracted with EA (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=I/O to 1/1) to give the title compound (1.00 g, 80% yield) as a yellow solid. ¹H NMR (400 MHz, DMSO-d₆) δ=8.38 (d, J=7.6 Hz, 1H), 7.41 (d, J=8.3 Hz, 2H), 7.27 (d, J=8.3 Hz, 2H), 6.40 (d, J=9.1 Hz, 1H), 5.11 (d, J=3.3 Hz, 1H), 4.85 (quin, J=7.1 Hz, 1H), 4.42 (t, J=7.9 Hz, 1H), 4.27 (s, 1H), 4.17-4.07 (m, 2H), 3.62-3.51 (m, 2H), 2.00-1.96 (m, 1H), 1.73 (ddd, J=4.6, 8.2, 12.7 Hz, 1H), 1.38 (s, 9H), 1.32 (d, J=7.0 Hz, 3H), 0.92 (s, 9H). LC-MS (ESI⁺) m/z 472.2 (M+H)⁺.

Step 2-(2S,4R)-1-((S)-2-amino-3,3-dimethylbutanoyl)-N—((S)-1-(4-ethynylphenyl)ethyl)-4-hydroxypyrrolidine-2-carboxamide. To a solution of tert-butyl N-[(1S)-1-[(2S,4R)-2-[[(1S)-1-(4-ethynylphenyl)ethyl]carbamoyl]-4-hydroxy-pyrrolidine-1-carbonyl]-2,2-dimethyl-propyl]carbamate (500 mg, 1.06 mmol) in DCM (6 mL) was added HCl/dioxane (4 M, 1.5 mL). The mixture was then stirred at 25° C. for 1 hour. On completion, the reaction mixture was concentrated under reduced pressure to give the title compound as a yellow solid (414 mg, HCl). LC-MS (ESI⁺) m/z 372.2 (M+H)⁺.

### (S)-4-(4-(2-(2-(2-hydroxyphenyl)-6a,7,9,10-tetrahydro-5H-pyrazino[1′,2′:4,5] pyrazino[2,3-c]pyridazin-8(6H)-yl)pyrimidin-5-yl)phenyl)cyclohexanecarboxylic acid (Intermediate FA)

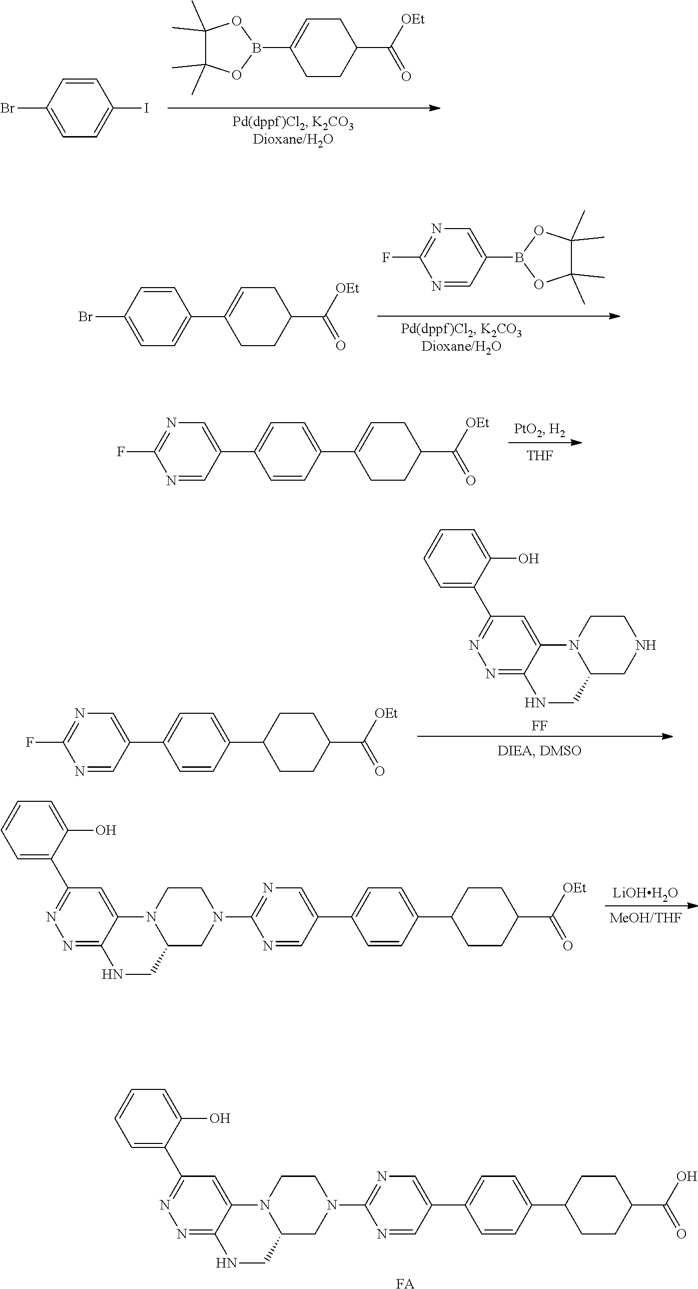

Step 1-Ethyl 4′-bromo-2,3,4,5-tetrahydro-[1,1′-biphenyl]-4-carboxylate. To a solution of 1-bromo-4-iodo-benzene (1.1 g, 3.9 mmol, CAS #589-87-7) and ethyl 4-(4,4,5,5-tetramethyl-1,3,2-dioxaborolan-2-yl)cyclohex-3-ene-1-carboxylate (1.1 g, 3.9 mmol, CAS #1049004-32-1) in dioxane (10 mL) and H₂O (4 mL) was added Pd(PPh₃)₄(224 mg, 194 umol) and K₂CO₃ (2 M, 4 mL). Then the mixture was stirred at 80° C. for 5 hours under N₂ atmosphere. On completion, the reaction mixture was quenched with H₂O (10 mL) at 25° C., and then extracted with EA (10 mL×3). The combined organic layers were washed with brine (10 mL×3), dried over with Na₂SO₄, then filtered and the filtration was concentrated under reduced pressure to give a residue. The residue was purified by column chromatography (SiO₂, Petroleum ether/Ethyl acetate=30/1 to 10/1) to give the title compound (870 mg, 72% yield) as a yellow oil. LC-MS (ESI, m/z): [M+1]⁺=309.1.

Step 2-Ethyl 4′-(2-fluoropyrimidin-5-yl)-2,3,4,5-tetrahydro-[1,1′-biphenyl]-4-carboxylate. To a solution of ethyl 4-(4-bromophenyl)cyclohex-3-ene-1-carboxylate (5 g, 16.1 mmol) in dioxane (50 mL) and H₂O (10 mL) was added 2-fluoro-5-(4,4,5,5-tetramethyl-1,3,2-dioxaborolan-2-yl)pyrimidine (7.25 g, 32.3 mmol, CAS #1352796-65-6), K₂CO₃ (6.70 g, 48.5 mmol) and Pd(dppf)Cl₂ (1.18 g, 1.62 mmol). Then the mixture was stirred at 85° C. for 12 hours. On completion, the residue was diluted with water (400 mL) and extracted with ethyl acetate (700 mL). The combined organic layers were washed with brine (500 mL), dried over Na₂SO₄, filtered and concentrated under reduced pressure to give a residue. The residue was purified by column chromatography (SiO₂, Petroleum ether/Ethyl acetate=10/1) to give title compound (4.6 g, 86% yield) as a yellow oil. LC-MS (ESI⁺) m/z 327.1(M+H)⁺.

Step 3-Ethyl 4-(4-(2-fluoropyrimidin-5-yl)phenyl)cyclohexanecarboxylate. To a solution of ethyl 4-[4-(2-fluoropyrimidin-5-yl)phenyl]cyclohex-3-ene-1-carboxylate (4.5 g, 13.7 mmol,) in THF (10 mL) was added PtO₂ (3.13 g, 13.7 mmol). Then the mixture was stirred under H₂ (45 Psi) at 30° C. for 12 hours. On completion, the reaction mixture was filtered and concentrated under reduced pressure to give a residue. The residue was purified by column chromatography (SiO₂, Petroleum ether/Ethyl acetate=8/1) to give (4 g, 84% yield) as a white solid. LC-MS (ESI⁺) m/z 329.1(M+H)⁺.

Step 4 (S)-ethyl 4-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)phenyl)cyclohexanecarboxylate. To a solution of 2-[(10R)-1,5,6,8,12-pentazatricyclo[8.4.0.0^{2,7}]tetradeca-2,4,6-trien-4-yl]phenol (670 mg, 2.36 mmol), and ethyl 4-[4-(2-fluoropyrimidin-5-yl)phenyl]cyclohexanecarboxylate (1.01 g, 3.07 mmol, Intermediate FF) in DMSO (8 mL) was added DIEA (1.53 g, 11.8 mmol). Then the mixture was stirred at 100° C. for 12 hours. On completion, the residue was diluted with water (50 mL) and extracted with DCM (50 mL). The combined organic layers were washed with brine (40 mL), dried over Na₂SO₄, filtered and concentrated under reduced pressure to give a residue. The residue was purified by column chromatography (SiO₂, DCM: MeOH=50:1) to give (850 mg, 53% yield) as a yellow solid. LC-MS (ESI⁺) m/z 592.2 (M+H)⁺.

Step 5-(S)-4-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)phenyl)cyclohexanecarboxylic acid. To a solution of ethyl 4-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]phenyl]cyclohexanecarboxylate (800 mg, 1.35 mmol) in THF (8 mL) and MeOH (3 mL) was added LiOH—H₂O (2 M, 2.70 mL), then the mixture was stirred at 25° C. for 12 hours, On completion, the mixture was added 1 N HCl until the pH=7. The residue was diluted with water (40 mL) and extracted with DCM (40 mL×2). The combined organic layers were washed with brine (40 mL), dried over Na₂SO₄ filtered and concentrated under reduced pressure to give the title compound (550 mg) as a white solid. LC-MS (ESI⁺) m/z 564.5 (M+H)⁺.

### 3-(2-(Methoxymethoxy)phenyl)-5,5-dimethyl-6,7,8,9-tetrahydro-5H-pyrido[3′,4′:4,5]pyrrolo[2,3-c]pyridazine (Intermediate FB)

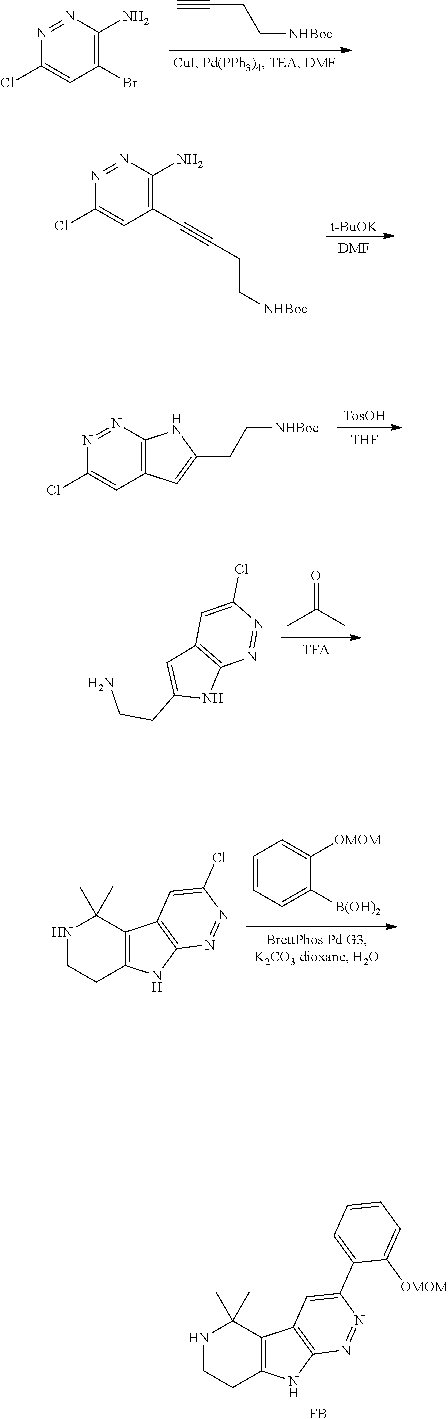

Step 1-Tert-butyl (4-(3-amino-6-chloropyridazin-4-yl)but-3-yn-1-yl)carbamate. A mixture of 4-bromo-6-chloro-pyridazin-3-amine (18 g, 86.3 mmol), tert-butyl N-but-3-ynylcarbamate (21.9 g, 130 mmol), TEA (87.4 g, 864 mmol, 120 mL), Pd(PPh₃)₄(4.99 g, 4.32 mmol) and CuI (1.64 g, 8.64 mmol) in DMF (300 mL) was degassed and purged with N₂ three times. Then the mixture was stirred at 35° C. for 2 hours under N₂ atmosphere. On completion, the reaction mixture was partitioned between ethyl acetate (600 mL) and water (500 mL). The organic phase was separated, washed with brine 500 mL (250 mL×2), dried over Na₂SO₄, filtered and concentrated under reduced pressure to give a residue. The residue was purified by flash silica gel chromatography (SiO₂, Petroleum ether/Ethyl acetate=10/1 to 1/1) to give the title compound (22.8 g, 83% yield) as a brown solid. ¹H NMR (400 MHz, DMSO-d₆) δ=7.45 (s, 1H), 7.12 (t, J=5.6 Hz, 1H), 6.79 (s, 2H), 3.19 (q, J=6.4 Hz, 2H), 2.61 (t, J=6.4 Hz, 2H), 1.37 (s, 9H). LC-MS (ESI⁺) m z 296.9 (M+H)⁺.

Step 2-Tert-butyl (2-(3-chloro-7H-pyrrolo[2,3-c]pyridazin-6-yl)ethyl)carbamate. To a solution of tert-butyl N-[4-(3-amino-6-chloro-pyridazin-4-yl)but-3-ynyl]carbamate (9.8 g, 33.0 mmol) in THF (100 mL) was added t-BuOK (4.45 g, 39.6 mmol). The mixture was then stirred from 0-25° C. for 2 hours. On completion, the reaction mixture was quenched with sat. NH₄Cl 300 mL at 0° C., and then diluted with ethyl acetate (400 mL) and extracted with water (150 mL×2). The combined organic layers were washed with brine (150 mL×2), filtered and concentrated under reduced pressure to give a residue. The crude product was triturated with Petroleum ether/Ethyl acetate=1/1 at 25° C. for 20 minutes, then filtered to give the title compound (7.5 g, 76% yield) as a brown solid. ¹H NMR (400 MHz, DMSO-d₆) δ=12.46 (s, 1H), 7.83 (s, 1H), 6.98 (t, J=5.2 Hz, 1H), 6.30 (s, 1H), 3.38-3.34 (m, 1H), 3.32 (s, 1H), 2.94 (t, J=6.8 Hz, 2H), 1.34 (s, 9H). LC-MS (ESI⁺) m/z 297.0 (M+H)⁺.

Step 3-2-(3-Chloro-7H-pyrrolo[2,3-c]pyridazin-6-yl)ethanamine. To a solution of tert-butyl N-[2-(3-chloro-7H-pyrrolo[2,3-c]pyridazin-6-yl)ethyl]carbamate (1 g, 3.37 mmol) in THF (10 mL) was added TosOH (1.16 g, 6.74 mmol). The mixture was then stirred at 70° C. for 12 hours. On completion, the reaction mixture was filtered and concentrated under reduced pressure to give the title compound (1 g) as a brown solid. LC-MS (ESI⁺) m/z 197.1 (M+H)⁺.

Step 4-3-Chloro-5,5-dimethyl-6,7,8,9-tetrahydro-5H-pyrido[3′,4′:4,5]pyrrolo[2,3-c]pyridazine. To a solution of 2-(3-chloro-7H-pyrrolo[2,3-c]pyridazin-6-yl)ethanamine (2.5 g, 12.7 mmol) and acetone (19.8 g, 340 mmol, 25.0 mL) was added TFA (25 mL). Then the mixture was stirred at 80° C. for 12 hours. On completion, the reaction mixture was filtered and concentrated under reduced pressure to give a residue. The crude product was purified by reversed-phase HPLC (0.1% NH₃•H₂O condition) to give the title compound (3 g, 94% yield) as a brown solid. ¹H NMR (400 MHz, DMSO-d₆) δ=12.75 (s, 1H), 9.46 (s, 1H), 8.18 (s, 1H), 3.57 (d, J=4.8 Hz, 2H), 3.10 (t, J=6.0 Hz, 2H), 1.72 (s, 6H). LC-MS (ESI⁺) m/z 237.1 (M+H)⁺.

Step 5-3-(2-(Methoxymethoxy)phenyl)-5,5-dimethyl-6,7,8,9-tetrahydro-5H-pyrido[3′,4′:4,5]pyrrolo[2,3-c]pyridazine. A mixture of 12-chloro-3,3-dimethyl-4,8,10,11-tetrazatricyclo[7.4.0.0^{2,7}]trideca-1(9),2(7),10,12-tetraene (1 g, 4.22 mmol), [2-(methoxymethoxy)phenyl]boronic acid (1.15 g, 6.34 mmol), BrettPhos Pd G3 (383 mg, 422 umol), and K₂CO₃ (2.92 g, 21.1 mmol) in dioxane (50 mL) and H₂O (10 mL) was degassed and purged with N₂ three times. Then the mixture was stirred at 80° C. for 12 hours under N₂ atmosphere. On completion, the reaction mixture was partitioned between ethyl acetate (300 mL) and water (200 mL). The organic phase was separated, washed with brine (100 mL×2), dried over Na₂SO₄, filtered and concentrated under reduced pressure to give a residue. The residue was purified by column chromatography (SiO₂, DCM: MeOH=50:1 to 10:1) to give the title compound (570 mg, 37% yield) as a yellow solid. LC-MS (ESI⁺) m/z 339.1 (M+H)⁺.

### Ethyl 2-(3-hydroxyisoxazol-5-yl)-3-methylbutanoate (Intermediate FC)

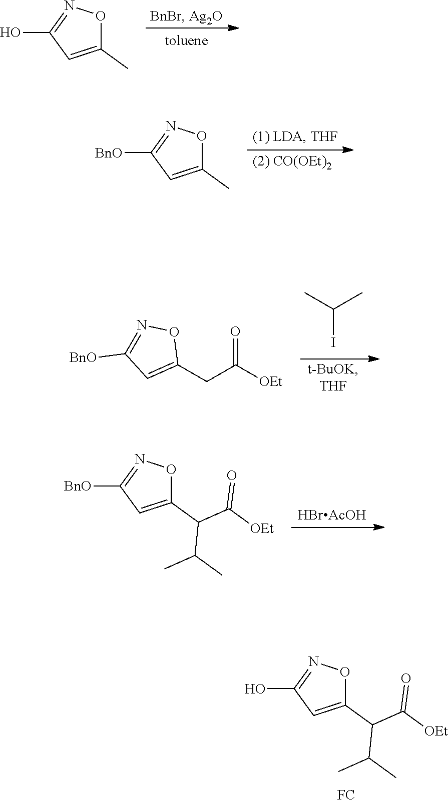

Step 1-3-(Benzyloxy)-5-methylisoxazole. To a solution of 5-methylisoxazol-3-ol (70 g, 0.71 mol, CAS #: 10004-44-1) in toluene (500 mL) was added BnBr (126 mL, 1.1 mol) and Ag₂CO₃ (273 g, 0.99 mol), then the mixture was stirred at 60° C. for 12 hours under N₂. On completion, the mixture was filtered and concentrated under reduced pressure to give a residue. The residue was purified by column chromatography (SiO₂, petroleum ether/ethyl acetate=50/1 to 10/1) to give the title compound (85 g, 64% yield) as a colorless oil. LC-MS (ESI, m/z): [M+1]⁺=190.0. ¹H NMR (400 MHz, CDCl₃) δ (ppm)=7.50-7.32 (m, 5H), 5.66 (d, J=0.6 Hz, 1H), 5.26 (s, 2H), 2.34 (s, 3H).

Step 2-Ethyl 2-(3-(benzyloxy)isoxazol-5-yl)acetate. To a solution of 3-benzyloxy-5-methyl-isoxazole (45 g, 0.24 mol) in THF (800 mL) was added LDA (2 M, 143 mL) at −78° C., then the mixture was stirred at −78° C. for 0.5 hours. Next, diethyl carbonate (42 g, 0.36 mmol, 43 mL.) was added to the mixture, and the mixture was stirred at −78° C. for 2.5 hr. On completion, the reaction mixture was quenched with sat. NH4Cl 200 mL at −70° C., and then diluted with EA (200 mL) and extracted with EA (500 mL×3). The combined organic layers were washed with sat. brine (500 mL), dried over Na₂SO₄, filtered and concentrated under reduced pressure to give a residue. The residue was purified by column chromatography (SiO₂, Petroleum ether/Ethyl acetate=I/O to 50/1) to give the title compound (23 g, 37% yield) as a colorless oil.) as a white solid. LC-MS (ESI, m/z): [M+1]⁺=262.1. ¹H NMR (400 MHz, CDCl₃) δ (ppm)=7.52-7.32 (m, 5H), 5.95 (s, 1H), 5.27 (s, 2H), 4.22 (q, J=7.1 Hz, 2H), 3.72 (s, 2H), 1.29 (t, J=7.1 Hz, 3H).

Step 3-Ethyl 2-(3-(benzyloxy)isoxazol-5-yl)-3-methylbutanoate. To a solution of ethyl 2-(3-benzyloxyisoxazol-5-yl)acetate (23 g, 88 mmol) in DMF (150 mL) was added t-BuOK (15 g, 0.13 mol) at 0° C., then 2-iodopropane (16 g, 92 mmol) was added to the mixture, and the mixture was stirred at 0° C. for 2 hrs. On completion, the reaction mixture was quenched with sat. NH₄Cl 100 mL at 0° C., and then diluted with EA (200 mL) and extracted with EA (200 mL×3). The combined organic layers were washed with sat. brine (200 mL), dried over Na₂SO₄, filtered and concentrated under reduced pressure to give the title compound (26 g) as a yellow oil. LC-MS (ESI, m/z): [M+1]⁺=304.0.

Step 4-Ethyl 2-(3-hydroxyisoxazol-5-yl)-3-methylbutanoate. A solution of ethyl 2-(3-benzyloxyisoxazol-5-yl)-3-methyl-butanoate (23 g, 76 mmol) in HBr (89 g, 0.33 mol, 30% solution) was stirred at 25° C. for 16 hrs. On completion, the reaction mixture was quenched with sat. NH₄Cl 80 mL, and then diluted with EA (50 mL) and extracted with EA (100 mL×3). The combined organic layers were washed with sat. brine (100 mL), dried over Na₂SO₄, filtered and concentrated under reduced pressure to give a residue. The residue was purified by column chromatography (SiO₂, Petroleum ether/Ethyl acetate=20/1 to 5/1) to give the title compound (14 g, 87% yield) as a yellow oil. LC-MS (ESI, m/z): [M+1]⁺=214.0; ¹H NMR (400 MHz, CDCl₃) δ (ppm)=5.95 (s, 1H), 4.28-4.12 (m, 2H), 3.45 (d, J=8.8 Hz, 1H), 1.28 (t, J=7.1 Hz, 3H), 1.02 (d, J=6.6 Hz, 3H), 0.93 (d, J=6.8 Hz, 3H).

Tert-butyl 2-(2-(methoxymethoxy)phenyl)-6a,7,9,10-tetrahydro-5H-pyrazino[1′,2′:4,5]pyrazino[2,3-c]pyridazine-8(6H)-carboxylate (Intermediate FD)

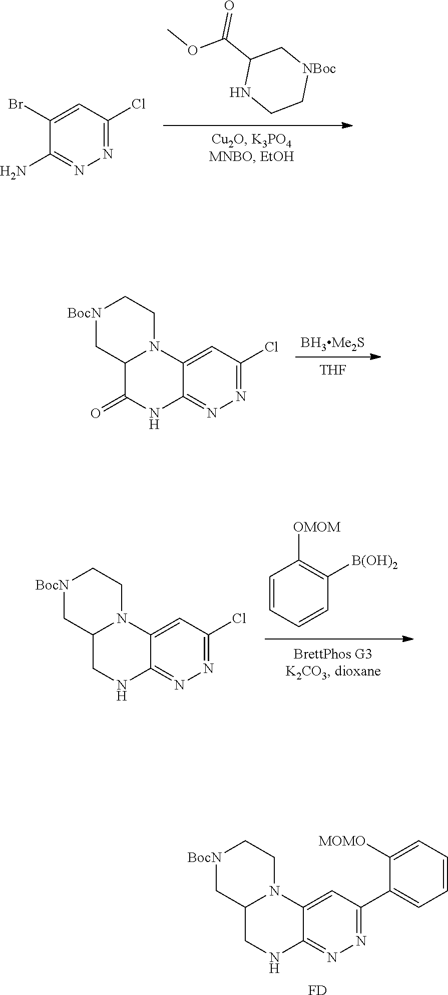

Step 1-Tert-butyl 2-chloro-6-oxo-6a,7,9,10-tetrahydro-5H-pyrazino[1′,2′:4,5]pyrazino[2,3-c]pyridazine-8(6H)-carboxylate. To a solution of 1-tert-butyl 3-methyl piperazine-1,3-dicarboxylate (105 g, 431 mmol, CAS #129779-08-2) in EtOH (1.6 L) saturated with 4-bromo-6-chloro-pyridazin-3-amine (60 g, 288 mmol, CAS #446273-59-2), N-benzyl-N′-(8-methyl-1-naphthyl)oxamide (9.16 g, 28.8 mmol), Cu₂O (8.24 g, 57.6 mmol) and K₃PO₄ (79.4 g, 374 mmol) was stirred at 120° C. for 16 hours in a 2 L of autoclave. On completion, the reaction solution was cooled to rt and the solution was poured out and filtered, then the filtration was concentrated to give a residue. The residue was purified by prep HPLC (0.1% FA condition) to give the title compound (60 g, 21% yield) as yellow solid. LC-MS (ESI⁺) m/z 340.2. (M+H)⁺.

Step 2 tert-butyl 2-chloro-6a,7,9,10-tetrahydro-5H-pyrazino[1′,2′:4,5]pyrazino[2,3-c]pyridazine-8(6H)-carboxylate. To a solution of tert-butyl 4-chloro-9-oxo-1,5,6,8,12-pentazatricyclo[8.4.0.02,7]tetradeca-2,4,6-triene-12-carboxylate (55 g, 162 mmol) in THF (1.1 L) was added BH₃-Me₂S (10 M, 80.9 mL) at 0° C. dropwise, then the mixture was stirred at 60° C. for 12 hours. On completion, the temperature was cooled down and quenched with MeOH (200 mL) dropwise, then the resulting solution was stirred at 60° C. for 12 hours. After this, the reaction temperature was cooled down then the solution was concentrated in vacuo to give a residue. The residue was purified by column chromatography (SiO₂, Petroleum ether/Ethyl acetate=5/1 to 1/1) to give the title compound (19 g, 36% yield) as white solid. LC-MS (ESI⁺) m/z 326.0. (M+H)⁺.

Step 3 tert-butyl 2-(2-(methoxymethoxy)phenyl)-6a,7,9,10-tetrahydro-5H-pyrazino[1′,2′:4,5]pyrazino[2,3-c]pyridazine-8(6H)-carboxylate. A solution of tert-butyl 4-chloro-1,5,6,8,12-pentazatricyclo[8.4.0.02,7]tetradeca-2,4,6-triene-12-carboxylate (15.1 g, 82.9 mmol), [2-(methoxymethoxy)phenyl]boronic acid (18 g, 55.3 mmol), BrettPhos Pd G3 (5.01 g, 5.52 mmol) and K₂CO₃ (22.9 g, 166 mmol) in dioxane (360 mL) and H₂O (72 mL) was degassed and then heated to 100° C. for 12 hours under N₂. On completion, the reaction mixture was extracted with ethyl acetate (3×100 mL). The combined organic layers were washed with brine (2×50 mL), and dried over anhydrous sodium sulfate, filtered and concentrated in vacuo to get the crude residue. The residue was purified by column chromatography (SiO₂, DCM/MeOH=20/1 to 10:1) to give the title compound (18 g, 76% yield) as yellow solid. LC-MS (ESI⁺) m/z 428.1. (M+H)⁺.

### (S)-tert-butyl 2-(2-(methoxymethoxy)phenyl)-6a,7,9,10-tetrahydro-5H-pyrazino[1′,2′:4,5]pyrazino[2,3-c]pyridazine-8(6H)-carboxylate (Intermediate FE)

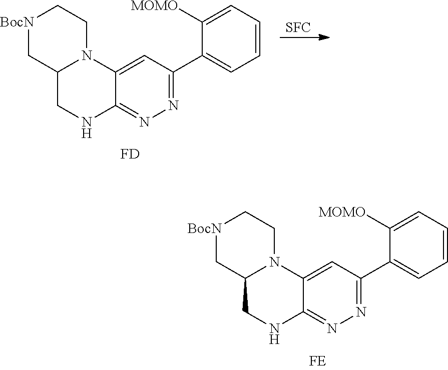

Tert-butyl 2-(2-(methoxymethoxy)phenyl)-6a,7,9,10-tetrahydro-5H-pyrazino[1′,2′:4,5]pyrazino[2,3-c]pyridazine-8(6H)-carboxylate (Intermediate FE) was purified by SFC (column: DAICEL CHIRALPAK AD (250 mm×50 mm, 10 um); mobile phase: [0.1% NH₃H2O EtOH]; B %: 35%-35%, 2.4; 500 min) to give the title compound 1 (7.2 g, 55% yield, structure confirmed) as yellow solid. ¹H NMR (400 MHz, CHLOROFORM-d) δ=7.72 (dd, J=1.6, 8.0 Hz, 1H), 7.37-7.31 (m, 1H), 7.21-7.16 (m, 1H), 7.12 (dt, J=1.0, 7.6 Hz, 1H), 6.92 (s, 1H), 5.17 (d, J=0.6 Hz, 2H), 4.27-4.08 (m, 3H), 3.69-3.58 (m, 2H), 3.43 (s, 3H), 3.42-3.36 (m, 1H), 3.33-3.25 (m, 1H), 3.10-2.98 (m, 1H), 2.89 (dt, J=3.6, 12.0 Hz, 1H), 2.76-2.50 (m, 2H), 2.05 (s, 1H), 1.49 (s, 9H).

### (R)-2-(6,6a,7,8,9,10-hexahydro-5H-pyrazino[1′,2′:4,5]pyrazino[2,3-c]pyridazin-2-yl)phenol (Intermediate FF)

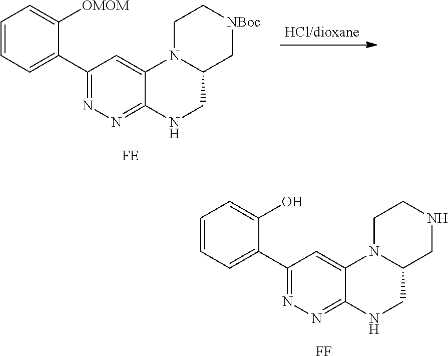

To a solution of tert-butyl (10S)-4-[2-(methoxymethoxy)phenyl]-1,5,6,8,12-pentazatricyclo[8.4.0.02,7]tetradeca-2,4,6-triene-12-carboxylate (1.5 g, 3.51 mmol, Intermediate FE) was added HCl/dioxane (4 M, 80 mL) at 25° C., then the reaction was stirred at 25° C. for 2 hours. On completion, the reaction mixture was filtered and concentrated in vacuo to give the title compound (1.4 g) as brown solid. LC-MS (ESI⁺) m/z 284.1 (M+H)⁺.

### Tert-butyl 4-(2-fluoropyrimidin-5-yl)piperidine-1-carboxylate (Intermediate FG)

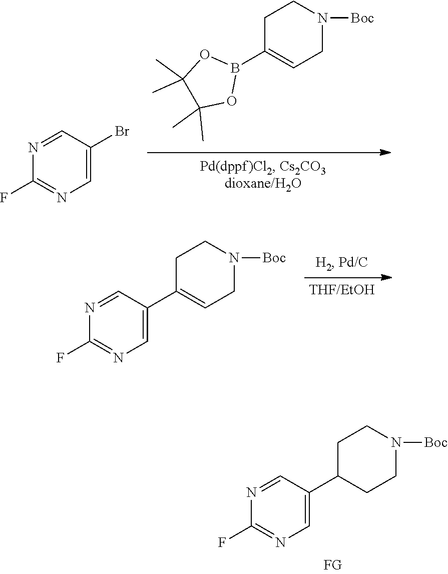 [SMILES: Fc1ncc(Br)cn1 ; CC(C)(C)OC(=O)N1CC=C(B2OC(C)(C)C(C)(C)O2)CC1 ; CC(C)(C)OC(=O)N1CC=C(c2cnc(F)nc2)CC1 ; CC(C)(C)OC(=O)N1CCC(c2cnc(F)nc2)CC1]

Step 1-Tert-butyl 4-(2-fluoropyrimidin-5-yl)-5,6-dihydropyridine-1(2H)-carboxylate. A mixture of 5-bromo-2-fluoro-pyrimidine (50 g, 282 mmol), tert-butyl 4-(4,4,5,5-tetramethyl-1,3,2-dioxaborolan-2-yl)-3,6-dihydro-2H-pyridine-1-carboxylate (104 g, 339 mmol), Pd(dppf)C1₂ (10.3 g, 14.1 mmol), Cs₂CO₃ (184. g, 565 mmol) in 1.4-dioxane (1000 mL) and H₂O (250 mL) was degassed and purged with N₂ three times. Then the mixture was stirred at 60° C. for 2.5 hours under N₂ atmosphere. On completion, the reaction mixture was diluted with H₂O (500 mL) and extracted with EA (1000 mL×3). The combined organic layers were dried over Na₂SO₄ filtered and concentrated under reduced pressure to give an oil. The residue was purified by MPLC(SiO₂, Petroleum ether/Ethyl acetate=5/1 to 3/1) to give the title compound (76 g, 86% yield) as a white solid. LC-MS (ESI⁺) m/z 280.1 (M+H)⁺.

Step 2-tert-butyl 4-(2-fluoropyrimidin-5-yl)piperidine-1-carboxylate. To a solution of tert-butyl 4-(2-fluoropyrimidin-5-yl)-3,6-dihydro-2H-pyridine-1-carboxylate (76 g, 272 mmol) in THF (200 mL) and EtOH (200 mL) was added Pd/C (10 wt %, 20 g) under N₂ atmosphere. The suspension was then degassed and purged with H₂ three times. The mixture was stirred under H₂ (30 Psi) at 25° C. for 12 hours. On completion, the reaction was filtered through celite very carefully, then the filtrate was concentrated under reduced pressure to give the title compound (72.6 g, 86% yield) as a white solid. ¹H NMR (400 MHz, DMSO-d₆) δ=8.48 (s, 2H), 4.29-4.28 (m, 2H), 2.85-2.71 (m, 3H), 1.88-1.85 (m, 2H), 1.68-1.61 (m, 2H), 1.47 (s, 9H).

### Ethyl 3-methyl-2-(3-(6-oxo-2-azaspiro[3.3]heptan-2-yl)isoxazol-5-yl)butanoate (Intermediate FH)

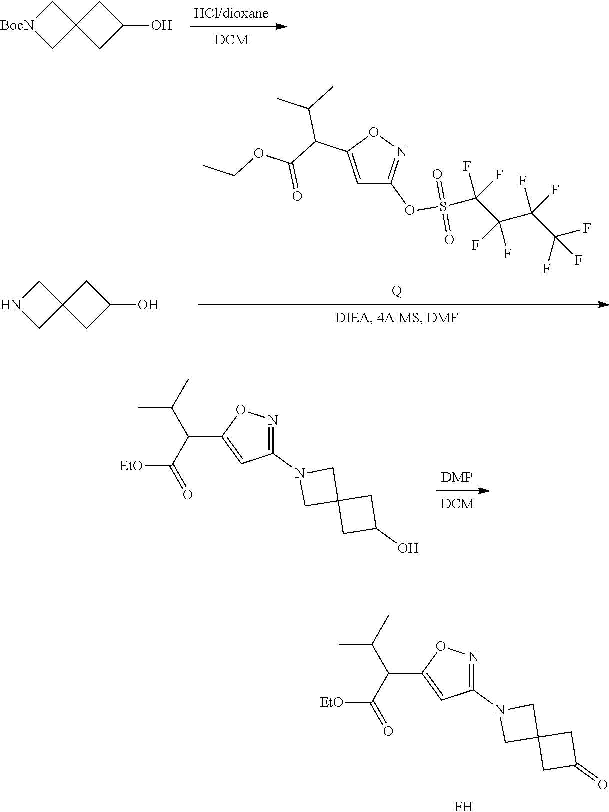

Step 1-2-Azaspiro[3.3]heptan-6-ol. To a solution of tert-butyl 6-hydroxy-2-azaspiro[3.3]heptane-2-carboxylate (995 mg, 4.67 mmol, CAS #1147557-97-8) in DCM (20 mL) was added HCl/dioxane (4 M, 10 mL). The mixture was stirred at 20° C. for 2 hours. On completion, the mixture was concentrated under reduced pressure to give the title compound (430 mg, HCl) as a light yellow gum. LC-MS (ESI⁺) m/z 114.1 (M+H)⁺.

Step 2-Ethyl 2-(3-(6-hydroxy-2-azaspiro[3.3]heptan-2-yl)isoxazol-5-yl)-3-methylbutanoate. To a solution of ethyl 3-methyl-2-(3-(((perfluorobutyl)sulfonyl)oxy)isoxazol-5-yl)butanoate (2.00 g, 4.04 mmol, Intermediate Q) in DMF (10 mL) was added DIEA (1.57 g, 12.1 mmol, 2.11 mL), 2-azaspiro[3.3]heptan-6-ol (906 mg, 6.06 mmol, HCl) and 4 Å molecular sieves (4 g). The mixture was stirred at 130° C. for 1 hour. On completion, the reaction mixture was filtered and quenched with water (40 mL) at 20° C., then extracted with EtOAc (20 mL×3). The combined organic layers were washed with brine (20 mL×3), dried over anhydrous sodium sulfate, filtered and concentrated under reduced pressure to give a residue, The residue was purified by column chromatography (SiO₂, Petroleum ether/Ethyl acetate=5/1 to 2/1) to give the title compound (1.8 g, 72% yield) as a yellow oil. LC-MS (ESI⁺) m/z 309.0 (M+H)⁺.

Step 3-Ethyl 3-methyl-2-(3-(6-oxo-2-azaspiro[3.3]heptan-2-yl)isoxazol-5-yl)butanoate. To a solution of DMP (2.81 g, 6.62 mmol, 2.05 mL) in DCM (40 mL) was added ethyl 2-[3-(6-hydroxy-2-azaspiro[3.3]heptan-2-yl)isoxazol-5-yl]-3-methyl-butanoate (1.7 g, 5.51 mmol) at 0° C. The mixture was then stirred at 20° C. for 3 hours. On completion, the reaction mixture was quenched with NaS₂O₃ aqueous solution (20 mL) and saturated NaHCO₃ aqueous solution (20 mL) at 20° C., and then extracted with DCM (30 mL×3). The combined organic layer was washed with saturated NaHCO₃ aqueous solution (50 mL) four times, dried over anhydrous sodium sulfate, filtered and concentrated under reduced pressure to give a residue. The residue was purified by column chromatography (SiO₂, Petroleum ether/Ethyl acetate=5/1 to 2/1) to give the title compound (1.3 g, 2.72 mmol, 49% yield) as a white solid. LC-MS (ESI⁺) m/z 307.2 (M+H)⁺.

### Tert-butyl 4-(2-chloropyrimidin-4-yl)piperazine-1-carboxylate (Intermediate FI) (CAS #221050-88-0

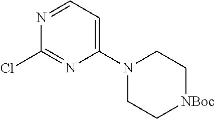 [SMILES: CC(C)(C)OC(=O)N1CCN(c2ccnc(Cl)n2)CC1]

### (R)-2-(6-(5-(1-(2-azaspiro[3.3]heptan-6-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate FJ)

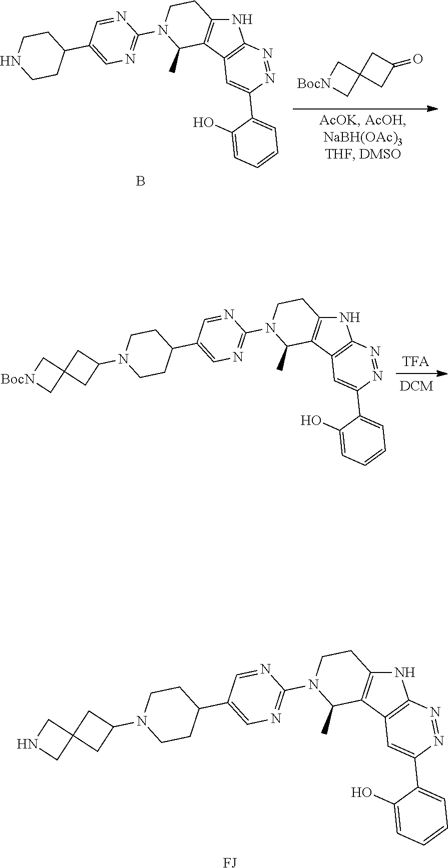

Step 1-(R)-tert-butyl 6-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate. To a solution of 2-[(3R)-3-methyl-4-[5-(4-piperidyl)pyrimidin-2-yl]-4,8,10,11-tetrazatricyclo[7.4.0.02,7]trideca-1(9),2(7),10,12-tetraen-12-yl]phenol (400 mg, 906 umol, Intermediate B) in THF (4 mL) and DMSO (2 mL) was added AcOK (267 mg, 2.72 mmol). The mixture was stirred at 25° C. for 0.5 hr. Then tert-butyl 6-oxo-2-azaspiro[3.3]heptane-2-carboxylate (230 mg, 1.09 mmol, CAS #1181816-12-5) and AcOH (218 mg, 3.62 mmol) was added in the mixture and stirred at 25° C. for 0.5 hour. Next, NaBH(OAc)₃ (576 mg, 2.72 mmol) was added to the reaction mixture at 0° C. and then the mixture was stirred at 25° C. for 3 hours. On completion, the reaction mixture was quenched with water (1 mL) and concentrated in vacuo to give a residue. The crude product was purified by prep-HPLC (0.10% FA condition) to give the title compound (350 mg, 59% yield, FA salt) as a white solid. LC-MS (ESI+) m/z 637.4 (M+H)⁺.

Step 2-(R)-2-(6-(5-(1-(2-azaspiro[3.3]heptan-6-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 6-[4-[2-[(3R)-12-(2-hydroxyphenyl)-3-methyl-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carboxylate (350 mg, 550 umol) in DCM (5 mL) was added TFA (2 mL). Then the mixture was stirred at 25° C. for 1 hour. On completion, the reaction mixture was concentrated in vacuo to give a residue. The crude product was purified by prep-HPLC (0.1% FA condition) to give the title compound (300 mg, 94% yield) as a white solid. LC-MS (ESI+) m/z 537-5 (M+H)⁺.

### 2-(6,6a,7,8,9,10-Hexahydro-5H-pyrazino[1′,2′:4,5]pyrazino[2,3-c]pyridazin-2-yl)phenol (Intermediate FK)

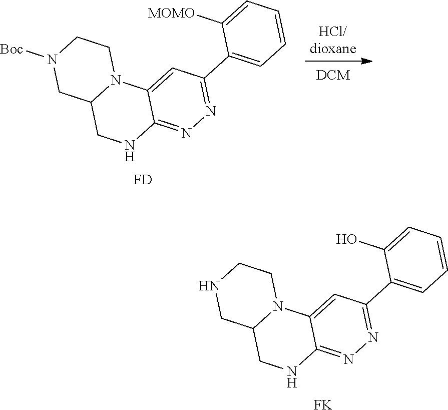

To a solution of tert-butyl 4-[2-(methoxymethoxy)phenyl]-1,5,6,8,12-pentazatricyclo[8.4.0.02,7]tetradeca-2,4,6-triene-12-carboxylate (5.2 g, 12.16 mmol, Intermediate FD) in DCM (60 mL) was added HCl/dioxane (4 M, 20 mL). Then the reaction mixture was stirred at 20° C. for 0.5 hour. On completion, the mixture was concentrated in vacuo to give the title compound (5 g) as a brown solid. LC-MS (ESI+) m/z 284.1 (M+H)⁺.

(R)-Tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (Intermediate FL) and (S)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (Intermediate FM)

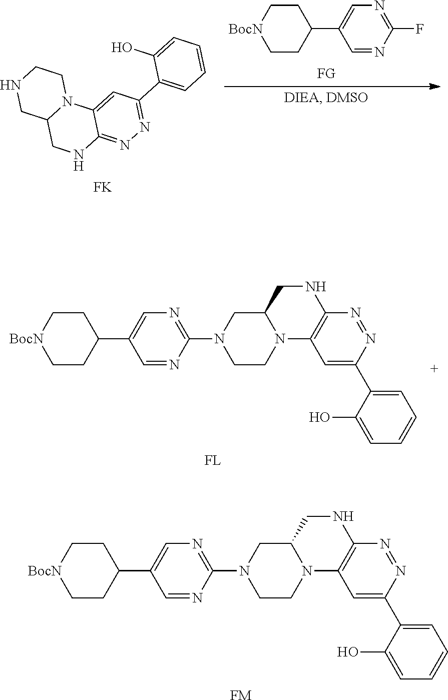

To a solution of 2-(1,5,6,8,12-pentazatricyclo[8.4.0.02,7]tetradeca-2,4,6-trien-4-yl)phenol (4 g, 12.5 mmol, Intermediate FK) in DMSO (50 mL) was added tert-butyl 4-(2-fluoropyrimidin-5-yl)piperidine-1-carboxylate (5.59 g, 18.7 mmol, Intermediate FG) and DIEA (8.08 g, 62.5 mmol). The reaction was stirred at 60° C. for 12 hours. On completion, the reaction mixture was poured into ice water (200 mL) and filtered to get the solid. Then the crude solid was purified by silica gel chromatography ((DCM:MeOH=100:0 to 15:1). Then the racemic purified product was separated by SFC (column: DAICEL CHIRALPAK AD (250 mm*50 mm, 10 um); mobile phase: [IPAACN];B %: 50%-50%, 5.5; 160 min) to give (R)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (1.3 g, 19% yield, SFC retention time: 1.420 min) as a brown solid, LC-MS (ESI+) m/z 284.1 (M+H); and (S)-tert-butyl 4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (2.0 g, 29% yield, SFC retention time: 2.204 min) as a brown solid, LC-MS (ESI+) m/z 284.1 (M+H)⁺.

### 2-[(3R)-3-Methyl-4-[5-[1-(4-piperidyl)-4-piperidyl]pyrimidin-2-yl]-4,8,10,11-tetrazatricyclo[7.4.0.0^{2,7}]trideca-1(9),2(7),10,12-tetraen-12-yl]phenol (Intermediate FN)

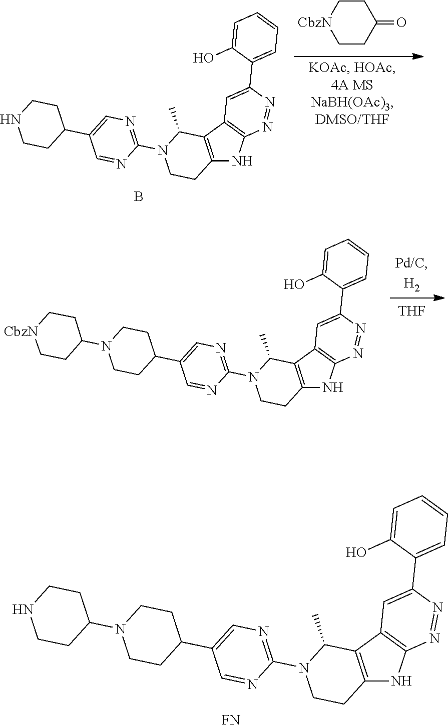

Step 1-(R)-benzyl 4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)-[1,4′-bipiperidine]-1′-carboxylate. To a solution of 2-[(3R)-3-methyl-4-[5-(4-piperidyl)pyrimidin-2-yl]-4,8,10,11-tetrazatricyclo[7.4.0.02,7]trideca-1(9),2(7),10,12-tetraen-12-yl]phenol (600 mg, 1.36 mmol, Intermediate O) in DMSO (1 mL) and THF (2 mL) was added KOAc (400 mg, 4.08 mmol), HOAc (244 mg, 4.08 mmol), 4 Å molecular sieves (200 mg) and benzyl 4-oxopiperidine-1-carboxylate (475 mg, 2.04 mmol, CAS #19099-93-5). The mixture was stirred at 0° C. for 1 hr, and then NaBH(OAc)₃ (864 mg 4.08 mmol) was added. The mixture was then stirred at 25° C. for 2 hours. On completion, the reaction mixture was quenched with H₂O (2 mL) at 0° C., then filtered and concentrated under reduced pressure to give a residue. The residue was purified by prep-HPLC (FA condition) to give the title compound (700 mg, 72% yield) as a yellow solid. LC-MS (ESI⁺) m/z 659.3 (M+H)⁺.

Step 2-(R)-2-(6-(5-([1,4′-bipiperidin]-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of benzyl 4-[4-[2-[(RS)-12-(2-hydroxyphenyl)-3-methyl-4,8,10,11-tetrazatricyclo[7.4.0.02,7]tridecal(9),2(7),10,12-tetraen-4-yl]pyrimidin-5-yl]-1-piperidyl]piperidine-1-carboxylate (600 mg, 910 umol) in THF (5 mL) was added Pd/C (10 wt %, 600 mg) under N₂. The suspension was degassed under vacuum and purged with H₂ several times. Then the mixture was stirred under H₂ (15 psi) at 20° C. for 12 hours. On completion, the reaction mixture was filtered and concentrated under reduced pressure to give the title compound (500 mg) as a yellow solid. LC-MS (ESI⁺) m/z 525.2 (M+H)⁺.

### (2S,4R)-1-((S)-2-amino-3,3-dimethylbutanoyl)-4-hydroxy-N-(4-(1-methyl-1H-pyrazol-5-yl)benzyl)pyrrolidine-2-carboxamide (Intermediate GL)

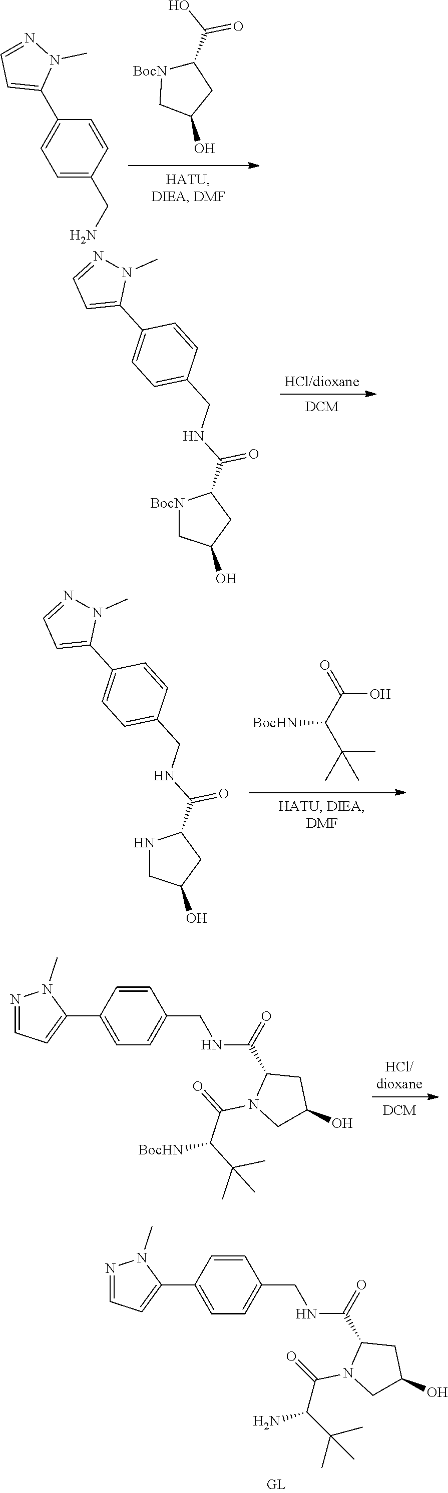

Step 1-(2S,4R)-tert-butyl 4-hydroxy-2-((4-(1-methyl-1H-pyrazol-5-yl)benzyl)carbamoyl)pyrrolidine-1-carboxylate. To a solution of (2S,4R)-1-tert-butoxycarbonyl-4-hydroxy-pyrrolidine-2-carboxylic acid (407.56 mg, 1.76 mmol, CAS #13726-69-7) in DMF (5 mL) was added HATU (792 mg, 2.08 mmol), DIEA (828 mg, 6.41 mmol) and [4-(2-methylpyrazol-3-yl)phenyl]methanamine (300 mg, 1.60 mmol, CAS #1340067-67-5). Then the mixture was stirred at 25° C. for 3 hours. On completion, The reaction mixture was diluted with water (15 mL) and extracted with EA (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=I/O to 0/1) to give compound (342 mg, 50% yield) as a yellow solid. LC-MS (ESI⁺) m/z 401.3 (M+H)⁺.

Step 2-(2S,4R)-4-hydroxy-N-(4-(1-methyl-1H-pyrazol-5-yl)benzyl)pyrrolidine-2-carboxamide. To a solution of tert-butyl (2S,4R)-4-hydroxy-2-[[4-(2-methylpyrazol-3-yl)phenyl]methylcarbamoyl]pyrrolidine-1-carboxylate (342 mg, 854.00 umol) in DCM (15 mL) was added HCl/dioxane (4 M, 5 mL). The mixture was then stirred at 25° C. for 2 hours. On completion, the reaction mixture was concentrated under reduced pressure to give the title compound (356 mg, HCl) as a yellow solid. LC-MS (ESI⁺) m/z 301.3 (M+H)⁺.

Step 3-Tert-butyl ((S)-1-((2S,4R)-4-hydroxy-2-((4-(1-methyl-1H-pyrazol-5-yl)benzyl)carbamoyl)pyrrolidin-1-yl)-3,3-dimethyl-1-oxobutan-2-yl)carbamate. To a solution of (2S)-2-(tert-butoxycarbonylamino)-3,3-dimethyl-butanoic acid (301 mg, 1.30 mmol) in DMF (8 mL) was added HATU (585 mg, 1.54 mmol), DIEA (612 mg, 4.74 mmol) and (2S,4R)-4-hydroxy-N-[[4-(2-methylpyrazol-3-yl)phenyl]methyl]pyrrolidine-2-carboxamide (356 mg, 1.19 mmol). The mixture was the stirred at 25° C. for 5 hours. On completion, the reaction mixture was diluted with H₂O (20 mL) and extracted with EA (30 mL×2). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=10/1 to 0/1) to give the title compound (288 mg, 42% yield) as a pink solid. LC-MS (ESI⁺) m/z 514.2 (M+H)⁺.

Step 4-(2S,4R)-1-((S)-2-amino-3,3-dimethylbutanoyl)-4-hydroxy-N-(4-(1-methyl-1H-pyrazol-5-yl)benzyl)pyrrolidine-2-carboxamide. To a solution of tert-butyl N-[(1S)-1-[(2S,4R)-4-hydroxy-2-[[4-(2-methylpyrazol-3-yl)phenyl]methylcarbamoyl]pyrrolidine-1-carbonyl]-2,2-dimethyl-propyl]carbamate (188 mg, 366 umol) in DCM (15 mL) was added HCl/dioxane (4 M, 91.51 uL). The mixture was then stirred at 25° C. for 2 hours. On completion, the reaction mixture was concentrated under reduced pressure to give the title compound (189 mg, HCl) as a yellow solid. LC-MS (ESI⁺) m/z 414.2 (M+H)⁺.

### (R)-7-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-4-yl)piperazin-1-yl)spiro[3.5]nonane-2-carboxylic acid (Intermediate GM)

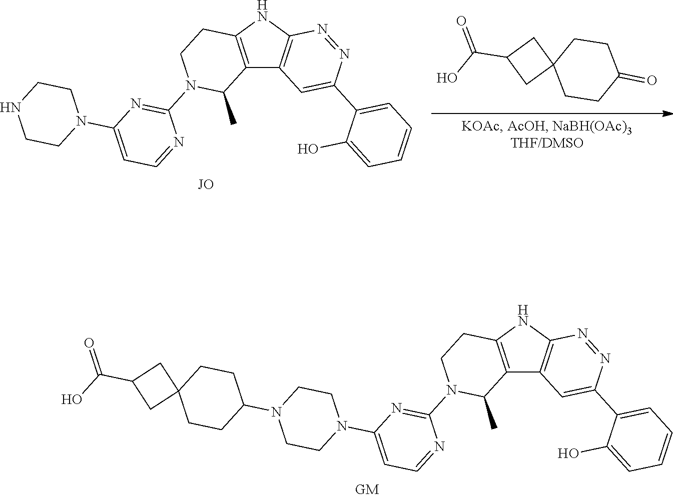 [SMILES: C[C@@H]1c2c([nH]c3nnc(-c4ccccc4O)cc23)CCN1c1nccc(N2CCNCC2)n1 ; O=C1CCC2(CC1)CC(C(=O)O)C2 ; C[C@@H]1c2c([nH]c3nnc(-c4ccccc4O)cc23)CCN1c1nccc(N2CCN(C3CCC4(CC3)CC(C(=O)O)C4)CC2)n1]

To a solution of 2-[(3R)-3-methyl-4-(4-piperazin-1-ylpyrimidin-2-yl)-4,8,10,11-tetrazatricyclo[7.4.0.02,7]trideca-1(9),2(7),10,12-tetraen-12-yl]phenol (350 mg, 790 umol, Intermediate JO) in THF (3.5 mL) and DMSO (1.75 mL) was added KOAc (233 mg, 2.37 mmol) then the reaction was stirred at 25° C. for 0.5 hr. Then 7-oxospiro [3.5] nonane-2-carboxylic acid (216 mg, 1.19 mmol, CAS #1440962-16-2) and HOAc (190 mg, 3.16 mmol) was added to the mixture and the mixture was stirred at 0° C. for 0.5 hr. Next, NaBH(OAc)₃ (335 mg, 1.58 mmol) was added and the reaction mixture was stirred at 25° C. for 1 hr. On completion, the reaction mixture was filtered and concentrated under reduced pressure to give a residue. The residue was purified by reversed phase flash chromatography (FA condition) to give the title compound (400 mg, 76% yield) as a yellow solid. ¹H NMR (400 MHz, DMSO-d₆) δ 12.45-12.38 (m, 1H), 8.66 (s, 1H), 8.21 (d, J=7.2 Hz, 1H), 8.13 (s, 1H), 7.92 (d, J=5.6 Hz, 1H), 7.35-7.25 (m, 1H), 7.00 (s, 2H), 6.09 (d, J=5.6 Hz, 1H), 6.05-5.91 (m, 1H), 5.12-4.99 (m, 1H), 3.63-3.49 (m, 4H), 3.11-3.04 (m, 1H), 3.00-2.96 (m, 1H), 2.95-2.92 (m, 1H), 2.89 (s, 1H), 2.26-2.21 (m, 2H), 2.20-2.16 (m, 2H), 2.14-2.08 (m, 2H), 2.04-1.98 (m, 2H), 1.87-1.83 (m, 4H), 1.77-1.73 (m, 2H), 1.63 (d, J=9.6 Hz, 2H), 1.51 (d, J=6.6 Hz, 3H), 1.26 (d, J=8.4 Hz, 2H). LC-MS (ESI⁺) m/z 609.2 (M+H)⁺.

### (2S,4R)-4-hydroxy-1-(2-(3-methoxyisoxazol-5-yl)-3-methylbutanoyl)pyrrolidine-2-carboxylic acid (Intermediate GN)

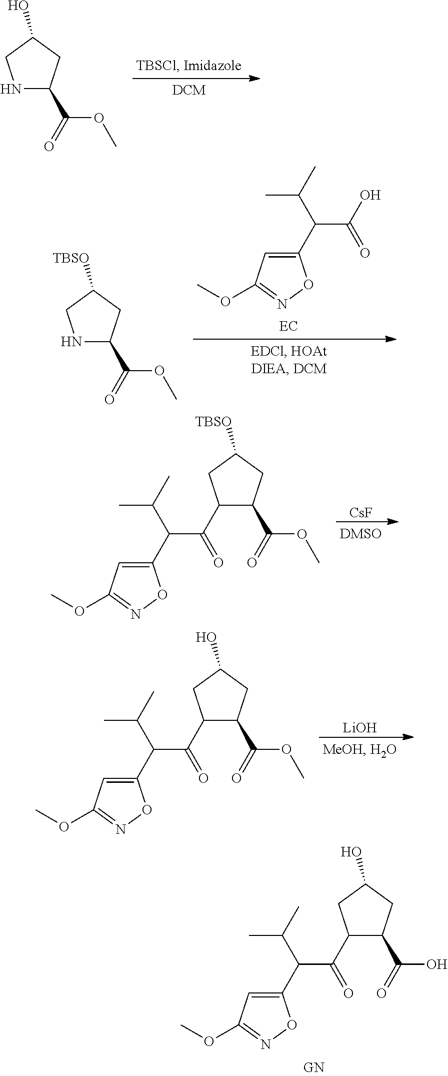

Step 1-(2S,4R)-methyl 4-((tert-butyldimethylsilyl)oxy)pyrrolidine-2-carboxylate. To a solution of methyl (2S,4R)-4-hydroxypyrrolidine-2-carboxylate (5 g, 34.5 mmol, CAS #1499-56-5) in DCM (50 mL) was added TBSCl (5.71 g, 37.9 mmol) and imidazole (4.69 g, 68.9 mmol). The mixture was stirred at 25° C. for 12 hours. The reaction mixture was extracted with DCM 150 mL (50 mL×3). The combined organic layers were washed with sat. NaCl 100 mL (100 mL×1), dried over Na₂SO₄, filtered and concentrated under reduced pressure to give the title compound (6 g) as a white solid. ¹H NMR (400 MHz, DMSO-d₆) δ=4.39-4.30 (m, 1H), 3.81 (t, J=7.6 Hz, 1H), 3.61 (s, 3H), 3.05 (dd, J=4.9 Hz, J=11.1 Hz, 1H), 2.62 (dd, J=2.5 Hz, J=11.1 Hz, 1H), 1.94-1.80 (m, 2H), 0.86-0.83 (m, 13H), 0.04 (d, J=2.6 Hz, 6H).

Step 2-(2S,4R)-methyl 4-((tert-butyldimethylsilyl)oxy)-1-(2-(3-methoxyisoxazol-5-yl)-3-methylbutanoyl)pyrrolidine-2-carboxylate. To a solution of 2-(aminomethyl)-5-ethynyl-phenol (0.8 g, 3.05 mmol, HCl) in DCM (8 mL) was added DIEA (1.23 g, 9.53 mmol), EDCI (548 mg, 2.86 mmol), HOAt (389 mg, 2.86 mmol) and (2S,4R)-4-hydroxy-1-[2-(3-methoxyisoxazol-5-yl)-3-methyl-butanoyl]pyrrolidine-2-carboxylic acid (595 mg, 1.91 mmol, Intermediate EC). The mixture was stirred at 25° C. for 12 hours. The mixture was concentrated under reduce pressure to give a residue. The crude product was purification by MPLC (SiO₂,PE:EA=1:1 to 1:2) to give the title compound (0.7 g, 78% yield) as a white solid. ¹H NMR (400 MHz, DMSO-d₆) δ=9.96-9.76 (m, 1H), 8.44-8.26 (m, 1H), 7.22-7.01 (m, 1H), 6.92-6.76 (m, 2H), 6.12-5.99 (m, 1H), 5.11 (dd, J=3.6 Hz, J=5.6 Hz, 1H), 4.50-4.29 (m, 2H), 4.25-4.10 (m, 2H), 4.09-4.04 (m, 1H), 3.85 (d, J=6.8 Hz, 3H), 3.75 (d, J=8.4 Hz, 1H), 3.66 (d, J=9.6 Hz, 1H), 3.60-3.48 (m, 1H), 3.43 (d, J=10.0 Hz, 1H), 2.29-2.18 (m, 1H), 2.01 (d, J=3.6 Hz, 1H), 1.89 (d, J=4.0 Hz, 1H), 1.00-0.90 (m, 3H), 0.81 (dd, J=6.8 Hz, J=13.2 Hz, 3H); LC-MS (ESI⁺) m z 442.3 (M+H)⁺.

Step 3-(2S,4R)-methyl 4-hydroxy-1-(2-(3-methoxyisoxazol-5-yl)-3-methylbutanoyl)pyrrolidine-2-carboxylate. To a solution of methyl (2S,4R)-4-[tert-butyl(dimethyl)silyl]oxy-1-[2-(3-methoxyisoxazol-5-yl)-3-methyl-butanoyl]pyrrolidine-2-carboxylate (22 g, 50 mmol) in DMSO (200 mL) was added CsF (37.9 g, 250 mmol). The mixture was stirred at 25° C. for 2 hours. On completion, the reaction mixture was quenched with H₂O (200 mL) at 25° C., and then extracted with EA 450 mL (150 mL×3). The combined organic layers were washed with brine 300 mL (100 mL×3), dried over Na₂SO₄, filtered and concentrated under reduced pressure to give the title compound (26 g) as a white solid. LC-MS (ESI⁺) m/z 327.1 (M+H)⁺.

Step 4-(2S,4R)-4-hydroxy-1-(2-(3-methoxyisoxazol-5-yl)-3-methylbutanoyl)pyrrolidine-2-carboxylic acid. To a solution of methyl (2S,4R)-4-hydroxy-1-[2-(3-methoxyisoxazol-5-yl)-3-methyl-butanoyl]pyrrolidine-2-carboxylate (21 g, 64 mmol) in H₂O (70 mL), MeOH (70 mL) and THF (70 mL) was added LiOH—H₂O (13.5 g, 322 mmol). The reaction mixture was diluted with H₂O (100 mL) and extracted with EA (100 mL×3). Then, 2M HCl was added into the combined water layers until the pH=1 and extracted with EA (100 mL×3). The organic layer was dried over Na₂SO₄, filtered and concentrated under reduced pressure to give a residue. The reaction mixture was concentrated under reduced pressure to give a residue. The crude product was purification by reversed-phase flash (0.1% HCl condition) to give the title compound (14 g, 66% yield, HCl) as white solid. ¹H NMR (400 MHz, DMSO-d₆) δ=12.37-12.05 (m, 1H), 6.11-6.00 (m, 1H), 5.20-5.12 (m, 1H), 4.30 (d, J=14.3 Hz, 1H), 4.25-4.14 (m, 1H), 3.89-3.81 (m, 3H), 3.78-3.72 (m, 1H), 3.50-3.42 (m, 1H), 2.30-2.19 (m, 1H), 2.15-2.04 (m, 1H), 1.91-1.90 (m, 2H), 0.95 (t, J=6.4 Hz, 3H), 0.81 (dd, J=4.0 Hz, J=6.8 Hz, 3H); LC-MS (ESI⁺) m/z 313.2 (M+H)⁺.

### Tert-butyl (3-(2-(4-bromobutoxy)phenyl)prop-2-yn-1-yl)carbamate (Intermediate GO)

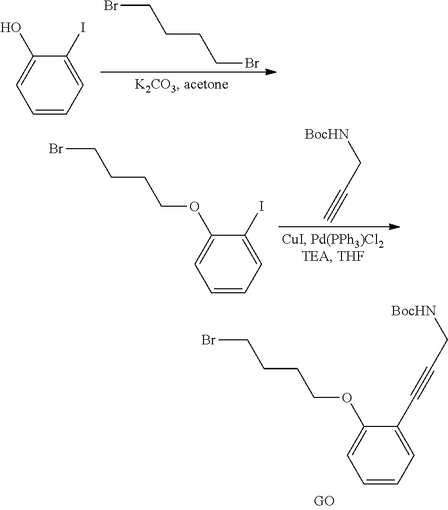

Step 1-1-(4-Bromobutoxy)-2-iodobenzene. To a solution of 2-iodophenol (1 g, 4.55 mmol) in acetone (10 mL) was added K₂CO₃ (1.26 g, 9.09 mmol) and 1,4-dibromobutane (4.91 g, 22.7 mmol, CAS #110-52-1). The mixture was stirred at 70° C. for 24 hours. On completion, the reaction mixture was filtered and concentrated under reduced pressure to give a residue. The residue was purified by column chromatography (SiO₂, Petroleum ether/Ethyl acetate=I/O to 80/1) to give the title compound (1.3 g, 80% yield) as a yellow oil. ¹H NMR (400 MHz, CHLOROFORM-d) δ=7.79 (dd, J=7.75, 1.50 Hz, 1H) 7.35-7.24 (m, 1H) 6.82 (dd, J=8.0, 4.0 Hz, 1H) 6.74 (td, J=8.0, 4.0 Hz, 1H) 4.08 (t, J=8.0 Hz, 2H) 3.57 (t, J=8.0 Hz, 2H) 2.26-2.11 (m, 2H) 2.09-1.99 (m, 2H).

Step 2-Tert-butyl (3-(2-(4-bromobutoxy)phenyl)prop-2-yn-1-yl)carbamate. To a solution of 1-(4-bromobutoxy)-2-iodo-benzene (1.3 g, 3.66 mmol) and tert-butyl N-prop-2-ynylcarbamate (1.14 g, 7.32 mmol, CAS #92136-39-5) in THF (10 mL) was added dichloropalladium;triphenylphosphane (257 mg, 366 umol), CuI (139 mg, 732 umol) and TEA (1.11 g, 10.9 mmol). The mixture was degassed and purged with N₂ for 3 times, and then the mixture was stirred at 25° C. for 2 hours under N₂ atmosphere. On completion, the reaction mixture was filtered and concentrated under reduced pressure to give a residue. The residue was purified by column chromatography (SiO₂, Petroleum ether/Ethyl acetate=100/1 to 80/1) to give the title compound (1.3 g, 93% yield) as a yellow solid. LC-MS (ESI⁺) m/z 326.1 (M−55+H)⁺.

### (S)-2-(8-(5-(1-(4-(2-(3-aminoprop-1-yn-1-yl)phenoxy)butyl)piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate GP)

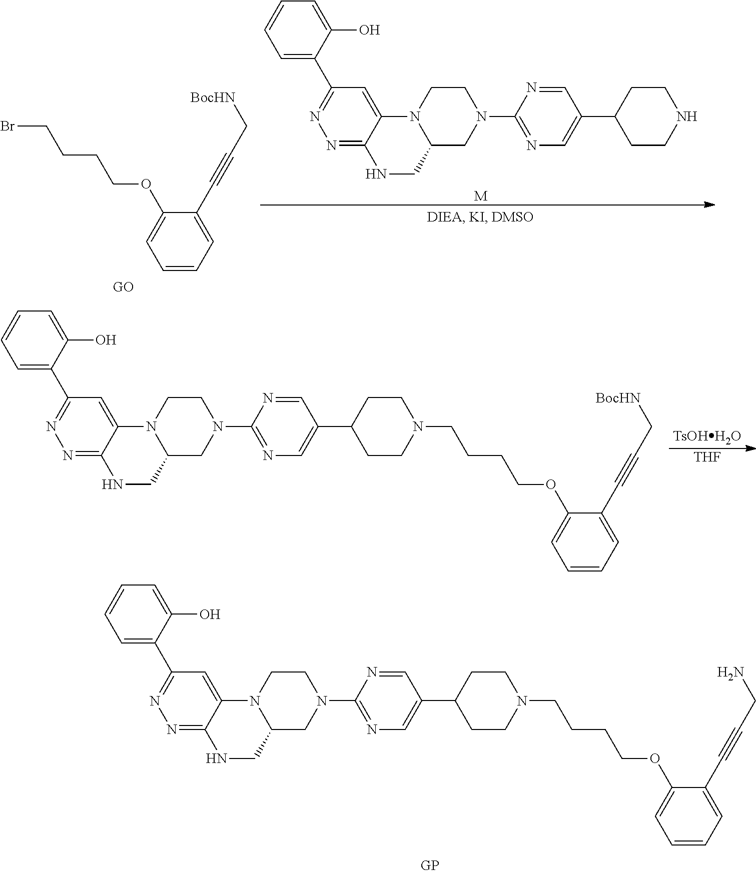

Step 1-(S)-tert-butyl (3-(2-(4-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)butoxy)phenyl)prop-2-yn-1-yl)carbamate. To a solution of tert-butyl N-[3-[2-(4-bromobutoxy)phenyl]prop-2-ynyl]carbamate (100 mg, 262 umol, Intermediate GO), 2-[(10S)-12-[5-(4-piperidyl)pyrimidin-2-yl]-1,5,6,8,12-pentazatricyclo[8.4.0.0^{2,7}]tetradeca-2,4,6-trien-4-yl]phenol (70 mg, 157 umol, Intermediate M) in DMSO (1 mL) was added DIEA (50.9 mg, 394 umol) and KI (43.6 mg, 262 umol). The mixture was stirred at 80° C. for 2 hours. On completion, the reaction mixture was filtered and concentrated under reduced pressure to give a residue. The residue was purified by prep-HPLC (FA condition) to give the title compound (100 mg, 87% yield, FA) as a yellow solid. LC-MS (ESI⁺) m/z 746.4 (M+H)⁺.

Step 2-(S)-2-(8-(5-(1-(4-(2-(3-aminoprop-1-yn-1-yl)phenoxy)butyl)piperidin-4-yl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl N-[3-[2-[4-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]butoxy]phenyl]prop-2-ynyl]carbamate (50 mg, 67.0 umol) in THF (1 mL) was added TsOH·H₂O (51 mg, 268 umol). The mixture was stirred at 70° C. for 12 hours. On completion, the reaction mixture was filtered and concentrated under reduced pressure to give a residue. The residue was purified by prep-HPLC (FA condition) to give the title compound (45 mg, 78% yield) as a white solid, LC-MS (ESI⁺) m/z 646.3 (M+H)⁺.

### (S)-2-(5-methyl-6-(5-(piperazin-1-yl)pyrimidin-2-yl)-6,7,8,9-tetrahydro-5H-pyrido[3′,4′:4,5]pyrrolo[2,3-c]pyridazin-3-yl)phenol (Intermediate IE) and (R)-2-(5-methyl-6-(5-(piperazin-1-yl)pyrimidin-2-yl)-6,7,8,9-tetrahydro-5H-pyrido[3′,4′:4,5]pyrrolo[2,3-c]pyridazin-3-yl)phenol (Intermediate GQ)

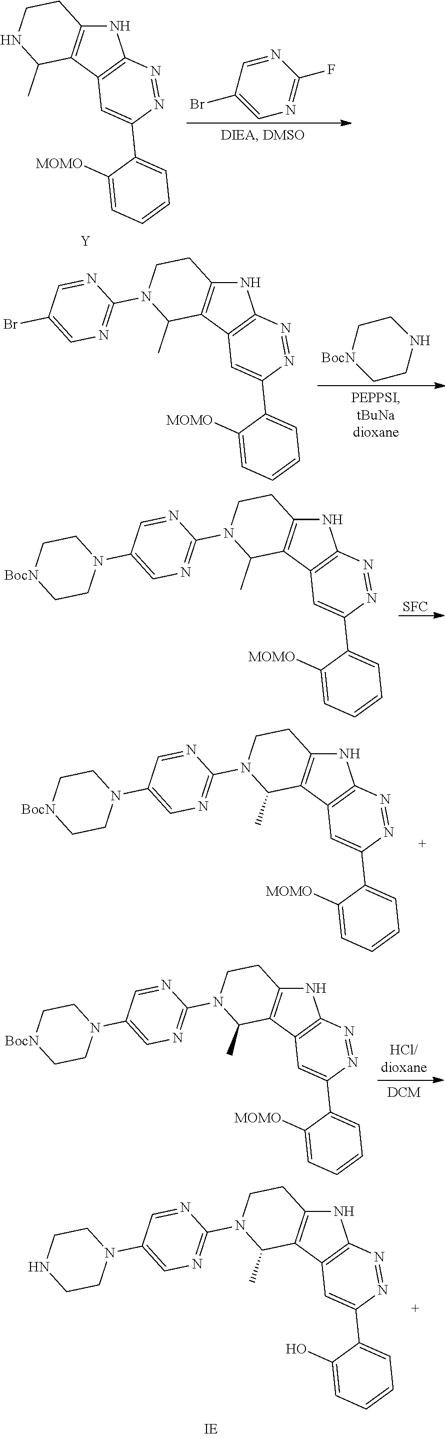

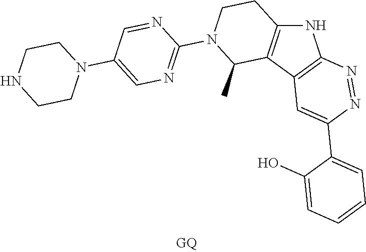 [SMILES: C[C@@H]1c2c([nH]c3nnc(-c4ccccc4O)cc23)CCN1c1ncc(N2CCNCC2)cn1]

Step 1-6-(5-Bromopyrimidin-2-yl)-3-(2-(methoxymethoxy)phenyl)-5-methyl-6,7,8,9-tetrahydro-5H-pyrido[3′,4′:4,5]pyrrolo[2,3-c]pyridazine. To a solution of 12-[2-(methoxymethoxy)phenyl]-3-methyl-4,8,10,11-tetrazatricyclo[7.4.0.0^{2,7}]trideca-1(9),2(7),10,12-tetraene (5 g, 15.4 mmol, Intermediate Y) in DMSO (100 mL) was added DIEA (5.98 g, 46.2 mmol) and 5-bromo-2-fluoro-pyrimidine (3.27 g, 18.5 mmol, CAS #62802-38-4). The mixture was stirred at 60° C. for 12 hours. On completion, the reaction mixture was diluted with H₂O (10 mL) and extracted with EA (50 mL×3). The organic layer was washed with brine (40 mL×2) and dried over Na₂SO₄. The organic layer concentrated in vacuo to give a residue. The residue was purified by column chromatography (SiO₂, Petroleum ether/Ethyl acetate=5/1 to 1/3) to give the title compound (5.6 g, 74% yield) as a yellow solid. LC-MS (ESI⁺) m/z 481.1 (M+H)⁺.

Step 2-Tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperazine-1-carboxylate. To a solution of 4-(5-bromopyrimidin-2-yl)-12-[2-(methoxymethoxy)phenyl]-3-methyl-4,8,10,11-tetrazatricyclo[7.4.0.0^{2,7}]trideca-1(9),2(7),10,12-tetraene (5.6 g, 11.6 mmol) in dioxane (60 mL) was added tert-butyl piperazine-1-carboxylate (4.33 g, 23.3 mmol, CAS #143238-38-4), tBuONa (2 M, 17.5 mL) and 1,3-bis[2,6-bis(1-ethylpropyl)phenyl]-2H-imidazole;3-chloropyridine;dichloropalladium (1.39 g, 1.75 mmol, CAS #1158652-41-5). The mixture was degassed and purged with N₂ three times, and then the mixture was stirred at 100° C. for 15 hours under N₂ atmosphere. On completion, the reaction mixture was diluted with H₂O (5 mL) and extracted with EA (20 mL×3). The combined organic layers were washed with brine (30 mL×2), dried over Na₂SO₄, filtered and concentrated under reduced pressure to give a residue. The residue was purified by column chromatography (SiO₂, Petroleum ether/Ethyl acetate=3/1 to 1/3) to give the title compound (6.7 g, 82% yield) as a white solid. LC-MS (ESI⁺) m/z 587.3 (M+H)⁺.

Step 3-(R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperazine-1-carboxylate (5-P1), (S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperazine-1-carboxylate. A solution of tert-butyl 4-[2-[12-[2-(methoxymethoxy)phenyl]-3-methyl-4,8,10,11-tetrazatricyclo[7.4.0.02,7]trideca-1(9),2(7),10,12-tetraen-4-yl]pyrimidin-5-yl]piperazine-1-carboxylate (6.5 g, 11.1 mmol) in MeOH (20 mL) was purified by SFC-separation (column: DAICEL CHIRALPAK AD(250 mm*30 mm, 10 um); mobile phase:[0.1% NH₃H2O IPA]; B %: 45%-45%, 5.5; 250 min) to give (R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperazine-1-carboxylate (2.5 g, 39% yield) as a yellow solid and (S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperazine-1-carboxylate (2.5 g, 38.5% yield) as a white solid (LC-MS (ESI⁺) m/z 587.3 (M+H)⁺). Absolute stereochemistry of the enantiomers was assigned arbitrarily.

Step 4-(S)-2-(5-methyl-6-(5-(piperazin-1-yl)pyrimidin-2-yl)-6,7,8,9-tetrahydro-5H-pyrido[3′,4′:4,5]pyrrolo[2,3-c]pyridazin-3-yl)phenol. To a solution of tert-butyl 4-[2-[(3S)-12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-5-yl]piperazine-1-carboxylate (2.2 g, 3.75 mmol) in DCM (20 mL) was added HCl/dioxane (4 M, 4 mL). The mixture was stirred at 25° C. for 12 hours. On completion, the reaction mixture was filtered and concentrated under reduced pressure to give a residue. The residue was purified by prep-HPLC (neutral condition; column: Waters xbridge 150*25 mm 10 um; mobile phase: [water(10 mM NH₄HCO₃)-ACN]; B %: 22%-52%, 11 min) to give the title compound (19.4 mg, 1% yield,) as a yellow solid, LC-MS (ESI⁺) m/z 443.2 (M+H)⁺.

Step 5-(R)-2-(5-methyl-6-(5-(piperazin-1-yl)pyrimidin-2-yl)-6,7,8,9-tetrahydro-5H-pyrido[3′,4′:4,5]pyrrolo[2,3-c]pyridazin-3-yl)phenol. To a solution of tert-butyl 4-[2-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-5-yl]piperazine-1-carboxylate (2.2 g, 3.75 mmol) in DCM (20 mL) was added HCl/dioxane (4 M, 6.00 mL). The mixture was stirred at 25° C. for 12 hours. On completion, the reaction mixture was filtered and concentrated under reduced pressure to give a residue. The residue was purified by prep-HPLC (neutral condition; column: Waters xbridge 150*25 mm 10 um; mobile phase: [water(10 mM NH₄HCO₃)-ACN]; B %: 22%-52%, 11 min) to give the title compound (25.5 mg, 2% yield,) as a yellow solid, LC-MS (ESI⁺) m/z 443.2 (M+H)⁺.

### (R)-6-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)spiro[3.3]heptane-2-carboxylic acid (Intermediate GR)

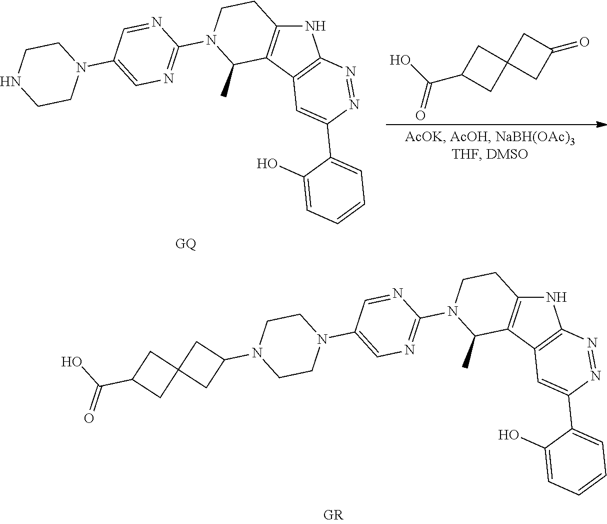 [SMILES: C[C@@H]1c2c([nH]c3nnc(-c4ccccc4O)cc23)CCN1c1ncc(N2CCNCC2)cn1 ; O=C1CC2(C1)CC(C(=O)O)C2 ; C[C@@H]1c2c([nH]c3nnc(-c4ccccc4O)cc23)CCN1c1ncc(N2CCN(C3CC4(CC(C(=O)O)C4)C3)CC2)cn1]

To a solution of (R)-2-(5-methyl-6-(5-(piperazin-1-yl)pyrimidin-2-yl)-6,7,8,9-tetrahydro-5H-pyrido[3′,4′:4,5]pyrrolo[2,3-c]pyridazin-3-yl)phenol (800 mg, 1.67 mmol, HCl, Intermediate GQ) in THF (3 mL) and DMSO (1 mL) was added AcOK (409 mg, 4.18 mmol) and the mixture was stirred for 30 minutes at 25° C. Then 2-oxospiro[3.3]heptane-6-carboxylic acid (515 mg, 3.34 mmol, CAS #889944-57-4) and AcOH (250 mg, 4.18 mmol) was added to the mixture which was then stirred for 1 hour. Next, NaBH(OAc)₃ (1.06 g, 5.01 mmol) was added at 0° C. The mixture was stirred at 25° C. for 1 hour. On completion, the reaction mixture was quenched with H₂O (10 mL) and then filtered and the filter was concentrated under reduced pressure to give the title compound (600 mg) as a yellow solid. LC-MS (ESI⁺) m z 581.2 (M+H)⁺.

### (R)-7-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)spiro[3.5]nonane-2-carboxylic acid (Intermediate GS)

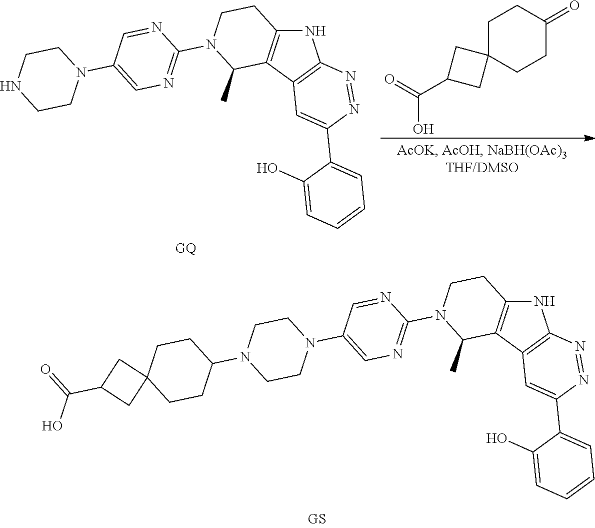

To a solution of 2-[(3S)-3-methyl-4-(5-piperazin-1-ylpyrimidin-2-yl)-4,8,10,11-tetrazatricyclo[7.4.0.0^{2,7}]trideca-1(9),2(7),10,12-tetraen-12-yl]phenol (800 mg, 1.08 mmol, Intermediate GQ) in THF (5 mL) and DMSO (1 mL) was added AcOK (319 mg, 3.25 mmol) and the mixture was stirred at 40° C. for 0.5 hour. Then 7-oxospiro[3.5]nonane-2-carboxylic acid (593 mg, 3.25 mmol) and AcOH (195 mg, 3.25 mmol, 186 uL) was added and stirred for another 1.5 hours. At last, NaBH(OAc)₃ (690 mg, 3.25 mmol) was added at 0° C. and the mixture was stirred at 25° C. for 12 hours. On completion, the reaction mixture was quenched with water (40 mL), then the suspension was filtered to give the title product (400 mg) as a yellow solid. LC-MS (ESI⁺) m/z 609.2 (M+H)⁺.

### (S)-2-(6-(5-(4-(2-azaspiro[3.3]heptan-6-yl)piperazin-1-yl)pyrimidin-2-yl)-5-methyl-6,7,8,9-tetrahydro-5H-pyrido[3′,4′:4,5]pyrrolo[2,3-c]pyridazin-3-yl)phenol (Intermediate GT)

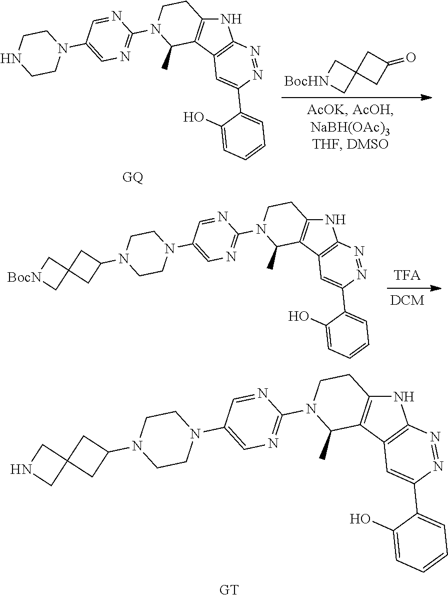

Step 1-(S)-tert-butyl 6-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)-2-azaspiro[3.3]heptane-2-carboxylate. To a solution of(S)-2-(5-methyl-6-(5-(piperazin-1-yl)pyrimidin-2-yl)-6,7,8,9-tetrahydro-5H-pyrido[3′,4′:4,5]pyrrolo[2,3-c]pyridazin-3-yl)phenol (400 mg, 904 umol, Intermediate GQ) in THF (4 mL) and DMSO (1 mL) was added KoAC (266 mg, 2.71 mmol). The mixture was stirred at 25° C. for 1 hour. Then tert-butyl 6-oxo-2-azaspiro[3.3]heptane-2-carboxylate (381 mg, 1.81 mmol, CAS #1181816-12-5) and AcOH (162 mg, 2.71 mmol) was added. The mixture was stirred at 25° C. for 2 hours. Then NaBH(OAc)₃ (479 mg, 2.26 mmol) was added at 0° C. Then the mixture was stirred at 25° C. for 1 hour. On completion, the reaction mixture was diluted with H₂O 10 mL and extracted with DCM (15×3 mL). The organic layer was dried over Na₂SO₄, concentrated in vacuo. The residue was purified by prep-HPLC (FA condition) to give the title compound (501 mg, 75% yield, FA) as a white solid. LC-MS (ESI⁺) m z 638.4 (M+H)⁺.

Step 2-(S)-2-(6-(5-(4-(2-azaspiro[3.3]heptan-6-yl)piperazin-1-yl)pyrimidin-2-yl)-5-methyl-6,7,8,9-tetrahydro-5H-pyrido[3′,4′:4,5]pyrrolo[2,3-c]pyridazin-3-yl)phenol. To a solution of (S)-tert-butyl 6-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)-2-azaspiro[3.3]heptane-2-carboxylate (501 mg, 785 umol) in DCM (5 mL) was added TFA (1 mL). The mixture was stirred at 25° C. for 2 hours. On completion, the reaction mixture was concentrated in vacuo to give the residue. The residue was purified by prep-HPLC (FA condition) to give the title compound (117 mg, 78% yield, FA) as an orange solid. LC-MS (ESI⁺) m z 538.3 (M+H)⁺.

### (2S,4R)-1-(2-amino-3,3-dimethylbutanoyl)-N-(3-(4-chlorophenyl)prop-2-yn-1-yl)-4-hydroxypyrrolidine-2-carboxamide (Intermediate GU)

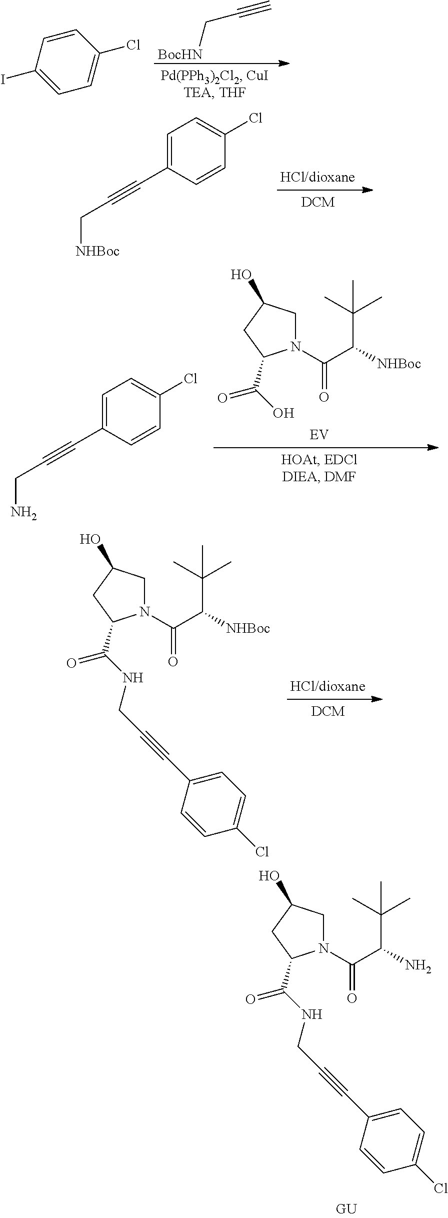

Step 1-Tert-butyl (3-(4-chlorophenyl)prop-2-yn-1-yl)carbamate. To a solution of 1-chloro-4-iodobenzene (2 g, 8.39 mmol) in THF was added tert-butyl prop-2-yn-1-ylcarbamate (2.60 g, 16.8 mmol), CuI (319 mg, 1.68 mmol), TEA (2.55 g, 25.2 mmol, 3.50 mL) and dichloropalladium;triphenylphosphane (589 mg, 839 umol). The mixture was then purged with N₂ for 3 times, and then the mixture was stirred at 25° C. for 2 hours under N₂ atmosphere. On completion, the reaction mixture was partitioned between H₂O (15 mL) and ethyl acetate (60 mL). The organic phase was separated, washed with brine (15 mL), dried over anhydrous sodium sulfate filtered and concentrated under reduced pressure to give a residue. The residue was purified by column chromatography (SiO₂, Petroleum ether/Ethyl acetate=10/1) to give the title compound (2.2 g, 98% yield) as a yellow solid. LC-MS (ESI⁺) m/z 210.1 (M−55)⁺.

Step 2-3-(4-Chlorophenyl)prop-2-yn-1-amine. To a solution of tert-butyl (3-(4-chlorophenyl)prop-2-yn-1-yl)carbamate (2.2 g, 8.28 mmol) in DCM (50 mL) was added HCl/dioxane (4 M, 5 mL). The mixture was then stirred at 25° C. for 2 hours. On completion, the reaction mixture was concentrated in vacuo to give the title compound (1.6 g) as a brown solid. LC-MS (ESI⁺) m/z 166.1 (M+H)⁺.

Step 3-Tert-butyl (1-((2S,4R)-2-((3-(4-chlorophenyl)prop-2-yn-1-yl)carbamoyl)-4-hydroxypyrrolidin-1-yl)-3,3-dimethyl-1-oxobutan-2-yl)carbamate. To a solution of (2S,4R)-1-((S)-2-((tert-butoxycarbonyl)amino)-3,3-dimethylbutanoyl)-4-hydroxypyrrolidine-2-carboxylic acid (1.70 g, 4.95 mmol, Intermediate EV) in DMF (15 mL) was added EDCI (1.42 g, 7.42 mmol) and HOBt (1.00 g, 7.42 mmol), then the mixture was stirred at 0° C. for 0.5 hour. Then 3-(4-chlorophenyl)prop-2-yn-1-amine (1 g, 4.95 mmol, HCl) and DIEA (3.20 g, 24.7 mmol, 4.31 mL) were added and the mixture was stirred at 25° C. for 1.5 hours. On completion, the reaction mixture was quenched by addition H₂O (50 mL) at 25° C., and then extracted with ethyl acetate (30 mL×3). The combined organic layers were washed with brine (30 mL×3), dried over dry Na₂SO₄, filtered and concentrated under reduced pressure to give a residue. The residue was purified by column chromatography (SiO₂, Petroleum ether/Ethyl acetate=1/2) to give the title compound (2.4 g, 90% yield) as a white solid. LC-MS (ESI⁺) m/z 492.4 (M+H)⁺.

Step 4-(2S,4R)-1-(2-amino-3,3-dimethylbutanoyl)-N-(3-(4-chlorophenyl)prop-2-yn-1-yl)-4-hydroxypyrrolidine-2-carboxamide. To a solution of tert-butyl (1-((2S,4R)-2-((3-(4-chlorophenyl)prop-2-yn-1-yl)carbamoyl)-4-hydroxypyrrolidin-1-yl)-3,3-dimethyl-1-oxobutan-2-yl)carbamate (500 mg, 1.02 mmol) in DCM (15 mL) was added HCl/dioxane (4 M, 5 mL). The mixture was stirred at 20° C. for 2 hours. On completion, the mixture was concentrated under reduced pressure to afford the title compound (430 mg) as yellow solid. LC-MS (ESI⁺) m/z 392.1 (M+H)⁺.

### (S)-2-(8-(5-(4-(2-azaspiro[3.3]heptan-6-yl)piperazin-1-yl)pyrimidin-2-yl)-6,6a,7,8,9,10-hexahydro-5H-pyrazino[1′,2′:4,5]pyrazino[2,3-c]pyridazin-2-yl)phenol (Intermediate GV)

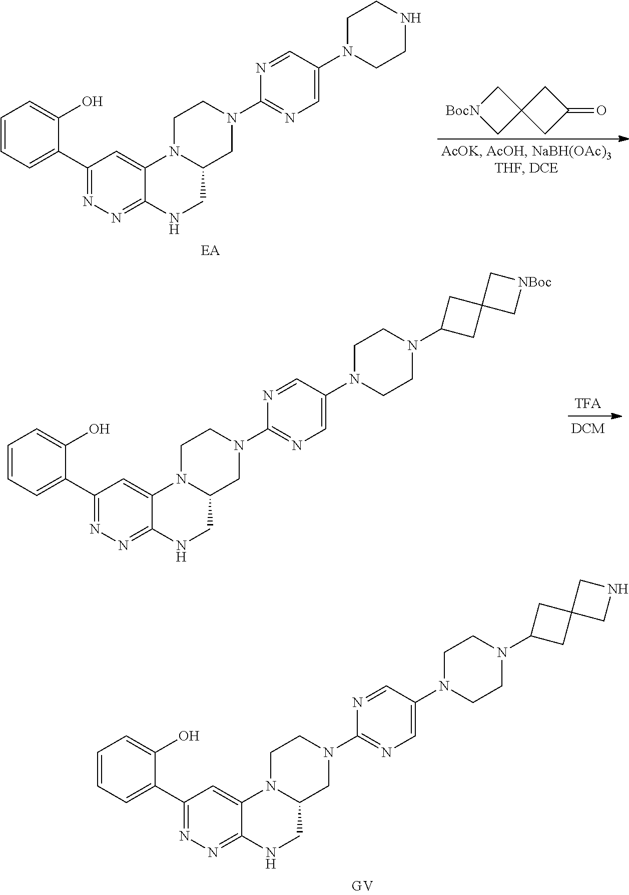 [SMILES: Oc1ccccc1-c1cc2c(nn1)NC[C@H]1CN(c3ncc(N4CCNCC4)cn3)CCN21 ; CC(C)(C)OC(=O)N1CC2(CC(=O)C2)C1 ; CC(C)(C)OC(=O)N1CC2(CC(N3CCN(c4cnc(N5CCN6c7cc(-c8ccccc8O)nnc7NC[C@H]6C5)nc4)CC3)C2)C1 ; Oc1ccccc1-c1cc2c(nn1)NC[C@H]1CN(c3ncc(N4CCN(C5CC6(CNC6)C5)CC4)cn3)CCN21]

Step 1-(S)-tert-butyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)-2-azaspiro[3.3]heptane-2-carboxylate. To a solution of (S)-2-(8-(5-(piperazin-1-yl)pyrimidin-2-yl)-6,6a,7,8,9,10-hexahydro-5H-pyrazino[1′,2′:4,5]pyrazino[2,3-c]pyridazin-2-yl)phenol (240 mg, 500 umol, HCl, Intermediate EA) in THF (4 mL) and DCE (1 mL) was added AcOK (146.61 mg, 1.49 mmol). The reaction was stirred at 25° C. for 30 minutes. Then AcOH (119.61 mg, 1.99 mmol) and tert-butyl 6-oxo-2-azaspiro[3.3]heptane-2-carboxylate (136.75 mg, 647.33 umol) was added at 25° C. and stirred for 1 hour. Last, NaBH(OAc)₃ (316.61 mg, 1.49 mmol) was added to the mixture at 0° C. The mixture was stirred at 25° C. for 1 hour. On completion, the reaction mixture was concentrated in vacuo to give a residue. The crude product was purified by column chromatography (SiO₂, Dichloromethane: Methanol=20/1 to 10/1) to give the title compound (140 mg, 29% yield) as a white solid. LC-MS (ESI⁺) m/z 641.3 (M+H)⁺.

Step 2-(S)-2-(8-(5-(4-(2-azaspiro[3.3]heptan-6-yl)piperazin-1-yl)pyrimidin-2-yl)-6,6a,7,8,9,10-hexahydro-5H-pyrazino[1′,2′:4,5]pyrazino[2,3-c]pyridazin-2-yl)phenol. To a solution of (S)-tert-butyl 6-(4-(2-(2-(2-hydroxyphenyl)-6a,7,9,10-tetrahydro-5H-pyrazino [1′,2′:4,5]pyrazino[2,3-c]pyridazin-8(6H)-yl)pyrimidin-5-yl)piperazin-1-yl)-2-azaspiro[3.3]heptane-2-carboxylate (140 mg, 220 umol) in DCM (2 mL) was added TFA (0.5 mL). The reaction mixture was stirred at 25° C. for 1 hour. On completion, the reaction mixture was concentrated in vacuo to give a residue. The crude product was purified by reversed-phase HPLC (0.1% FA condition) to give the title compound (50 mg, 38% yield, FA) as a pink solid. LC-MS (ESI⁺) m/z 541.4 (M+H)⁺.

### (S)-1-((2S,4R)-4-((tert-butyldimethylsilyl)oxy)-2-((4-(4-methylthiazol-5-yl)benzyl)carbamoyl)pyrrolidin-1-yl)-3,3-dimethyl-1-oxobutan-2-yl 1H-imidazole-1-carboxylate (Intermediate GW)

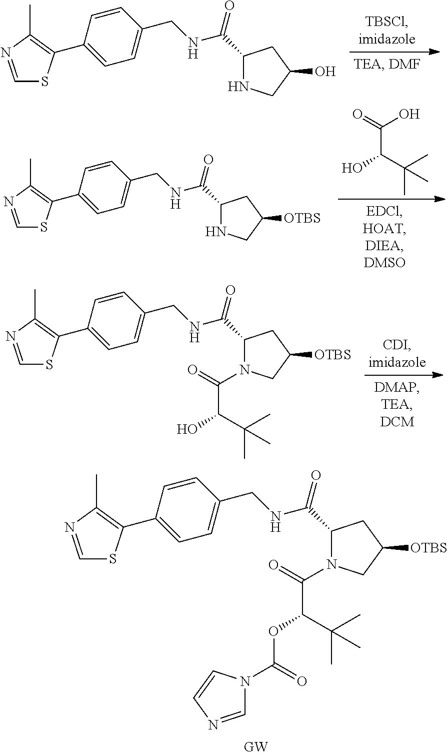

Step 1-(2S,4R)-4-((tert-butyldimethylsilyl)oxy)-N-(4-(4-methylthiazol-5-yl)benzyl)pyrrolidine-2-carboxamide. To a solution of (2S,4R)-4-hydroxy-N-(4-(4-methylthiazol-5-yl)benzyl)pyrrolidine-2-carboxamide (2.5 g, 7.9 mmol, CAS #1448189-90-9) in DCM (25 mL) was added TEA (2.39 g, 23.6 mmol) and stirred at 25° C. for 5 min. Then imidazole (1.07 g, 15.8 mmol) was added at 25° C. and TBSCl (2.37 g, 15.8 mmol) was added at 0° C. The reaction mixture was stirred at 25° C. for 14 hours. On completion, the reaction mixture was concentrated in vacuo to give a residue. The crude product was purified by column chromatography (SiO₂, Petroleum ether/Ethyl acetate=6/1 to 1/1) to give the title compound (1.05 g, 30% yield) as a yellow solid. LC-MS (ESI⁺) m/z 432.3 (M+H)⁺.

Step 2-(2S,4R)-4-((tert-butyldimethylsilyl)oxy)-1-((S)-2-hydroxy-3,3-dimethylbutanoyl)-N-(4-(4-methylthiazol-5-yl)benzyl)pyrrolidine-2-carboxamide. To a solution of (2S,4R)-4-((tert-butyldimethylsilyl)oxy)-N-(4-(4-methylthiazol-5-yl)benzyl)pyrrolidine-2-carboxamide (1 g, 2 mmol) in DMSO (10 mL) was added DIEA (898.22 mg, 6.95 mmol), EDCI (666.15 mg, 3.47 mmol), HOAT (472.97 mg, 3.47 mmol) and (2S)-2-hydroxy-3,3-dimethyl-butanoic acid (612.31 mg, 4.63 mmol). The reaction mixture was stirred at 25° C. for 1 hour. On completion, the reaction mixture was concentrated in vacuo to give a residue. The crude product was purified by column chromatography (SiO₂, Petroleum ether/Ethyl acetate=8/1 to 1/1) to give the title compound (973 mg, 70% yield) as a yellow solid. LC-MS (ESI⁺) m/z 546.5 (M+H). ¹H NMR (400 MHz, DMSO-d6) δ=8.92-8.91 (m, 1H), 8.50 (t, J=6.0 Hz, 1H), 7.39-7.27 (m, 4H), 6.64-6.64 (m, 1H), 4.50 (d, J=8.0 Hz, 1H), 4.46-4.39 (m, 2H), 4.34-4.26 (m, 1H), 4.22-4.14 (m, 1H), 3.83 (d, J=8.0 Hz, 1H), 3.61-3.50 (m, 2H), 2.37 (s, 3H), 1.92 (s, 1H), 1.88-1.80 (m, 1H), 0.85-0.80 (m, 9H), 0.80-0.75 (m, 9H), 0.03-0.04 (m, 6H).

Step 3-(S)-1-((2S,4R)-4-((tert-butyldimethylsilyl)oxy)-2-((4-(4-methylthiazol-5-yl)benzyl)carbamoyl)pyrrolidin-1-yl)-3,3-dimethyl-1-oxobutan-2-yl 1H-imidazole-1-carboxylate. To a solution of (2S,4R)-4-((tert-butyldimethylsilyl)oxy)-1-((S)-2-hydroxy-3,3-dimethylbutanoyl)-N-(4-(4-methylthiazol-5-yl)benzyl)pyrrolidine-2-carboxamide (50 mg, 92 umol) in DCM (0.5 mL) was added imidazole (18.71 mg, 274.8 umol) and DMAP (10.07 mg, 82.45 umol) at 25° C. and the CDI (29.71 mg, 183.21 umol) was added at 0° C. The reaction mixture was stirred at 25° C. for 3 hr. On completion, the reaction mixture was diluted with H₂O (10 mL) and extracted with EA (10 mL×3). The combined organic layers were washed with brine (15 mL×2), dried over Na₂SO₄, filtered and concentrated under reduced pressure to give the title compound (50 mg) as a yellow oil. LC-MS (ESI⁺) m/z 640.4 (M+H)⁺.

### (2S,4R)-1-((S)-2-amino-3,3-dimethylbutanoyl)-N-(4-ethynyl-2-fluorobenzyl)-4-hydroxypyrrolidine-2-carboxamide (Intermediate GX)

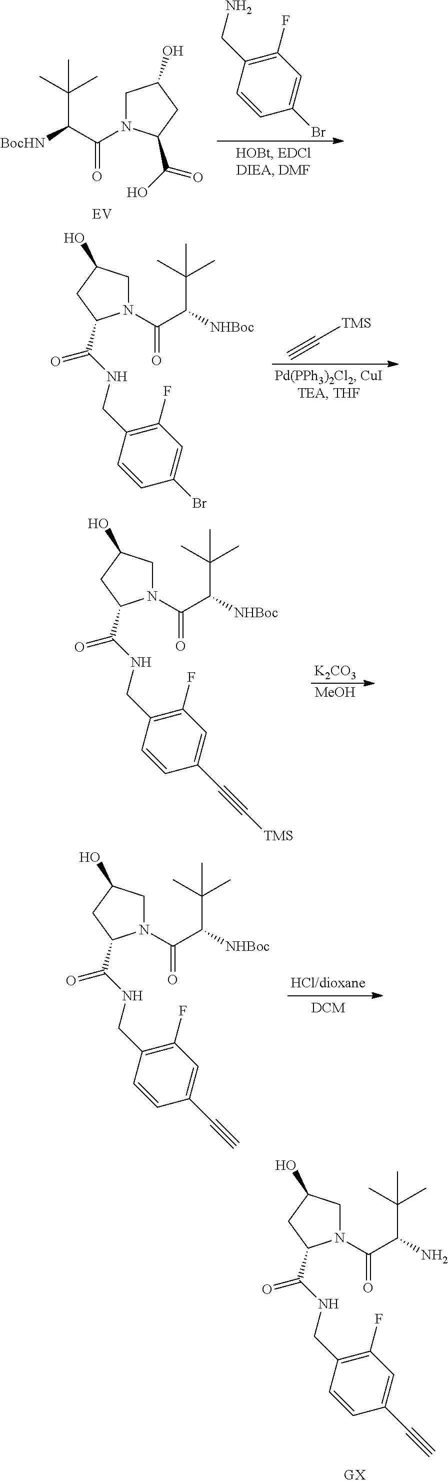

Step 1-Tert-butyl ((S)-1-((2S,4R)-2-((4-bromo-2-fluorobenzyl)carbamoyl)-4-hydroxypyrrolidin-1-yl)-3,3-dimethyl-1-oxobutan-2-yl)carbamate. To a solution of (4-bromo-2-fluorophenyl)methanamine (1 g, 4.9 mmol) in DMF (15 mL) was added HOBt (993 mg, 7.35 mmol) and EDCI (1.41 g, 7.35 mmol). The mixture was stirred at 0° C. for 0.5 hour. Then (2S,4R)-1-((S)-2-((tert-butoxycarbonyl)amino)-3,3-dimethylbutanoyl)-4-hydroxypyrrolidine-2-carboxylic acid (2.53 g, 7.35 mmol, Intermediate EV) and DIEA (3.17 g, 24.5 mmol, 4.27 mL) was added, then the mixture was stirred at 25° C. for 1.5 hours. On completion, the mixture was filtered to give a crude product. The crude product was purified by reversed-phase HPLC (0.1% FA condition) to give the title compound (1 g, 38% yield) as a white solid. LC-MS (ESI⁺) m/z 532.1 (M+H)⁺.

Step 2-Tert-butyl ((S)-1-((2S,4R)-2-((2-fluoro-4-((trimethylsilyl)ethynyl)benzyl)carbamoyl)-4-hydroxypyrrolidin-1-yl)-3,3-dimethyl-1-oxobutan-2-yl)carbamate. To a solution of tert-butyl ((S)-1-((2S,4R)-2-((4-bromo-2-fluorobenzyl)carbamoyl)-4-hydroxypyrrolidin-1-yl)-3,3-dimethyl-1-oxobutan-2-yl)carbamate (700 mg, 1.32 mmol) in TEA (5 mL) was added ethynyltrimethylsilane (1.30 g, 13.2 mmol, 1.83 mL), Pd(PPh₃)₂Cl₂ (139 mg, 198 umol), and CuI (75.4 mg, 396 umol) and purged with N₂ three times. Then the mixture was stirred at 60° C. for 12 hours under N₂ atmosphere. On completion, the mixture was filtered to give a crude product. The crude product was purified by reversed-phase HPLC (0.1% FA condition) to give the title compound (600 mg, 74% yield, FA) as a light white solid. LC-MS (ESI⁺) m/z 570.2 (M+Na)⁺.

Step 3-Tert-butyl ((S)-1-((2S,4R)-2-((4-ethynyl-2-fluorobenzyl)carbamoyl)-4-hydroxypyrrolidin-1-yl)-3,3-dimethyl-1-oxobutan-2-yl)carbamate. To a solution of tert-butyl ((S)-1-((2S,4R)-2-((2-fluoro-4-((trimethylsilyl)ethynyl)benzyl)carbamoyl)-4-hydroxypyrrolidin-1-yl)-3,3-dimethyl-1-oxobutan-2-yl)carbamate (600 mg, 1.10 mmol) in MeOH (20 mL) was added K₂CO₃ (303 mg, 2.19 mmol). The mixture was stirred at 20° C. for 3 hours. On completion, the mixture was quenched by adding H₂O (20 mL), extracted with EtOAc (10 mL×3) and washed with brine (10 mL×3). The mixture was concentrated under reduced pressure to give the title compound (550 mg) as a brown solid. LC-MS (ESI⁺) m/z 476.2 (M+H)⁺.

Step 4-(2S,4R)-1-((S)-2-amino-3,3-dimethylbutanoyl)-N-(4-ethynyl-2-fluorobenzyl)-4-hydroxypyrrolidine-2-carboxamide. To a solution of tert-butyl ((S)-1-((2S,4R)-2-((4-ethynyl-2-fluorobenzyl)carbamoyl)-4-hydroxypyrrolidin-1-yl)-3,3-dimethyl-1-oxobutan-2-yl)carbamate (250 mg, 526 umol) in DCM (15 mL) was added HCl/dioxane (4 M, 4 mL). The mixture was stirred at 20° C. for 2 hours. On completion, the mixture was concentrated under reduced pressure to give the title compound (200 mg) as a brown solid. LC-MS (ESI⁺) m/z 376.1 (M+H)⁺.

### (2S,4R)-1-((S)-2-amino-3,3-dimethylbutanoyl)-N-(3-(4-fluoro-2-methylphenyl)prop-2-yn-1-yl)-4-hydroxypyrrolidine-2-carboxamide (Intermediate GY)

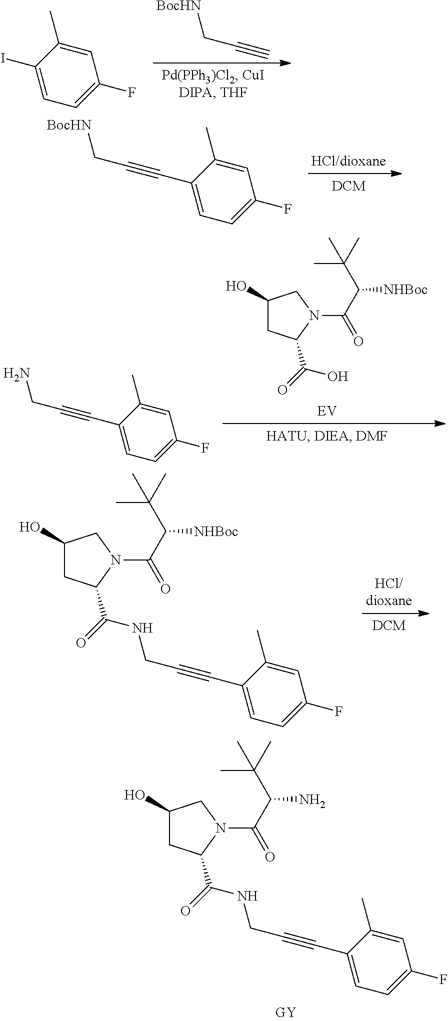

Step 1-Tert-butyl (3-(4-fluoro-2-methylphenyl)prop-2-yn-1-yl)carbamate. A mixture of 4-fluoro-1-iodo-2-methyl-benzene (4 g, 17 mmol), tert-butyl N-prop-2-ynylcarbamate (3.95 g, 25.4 mmol), Pd(PPh₃)₂Cl₂ (1.19 g, 1.69 mmol), CuI (645 mg, 3.39 mmol) and N-isopropylpropan-2-amine (5.14 g, 50.84 mmol, 7.19 mL) in THF (40 mL) was degassed and purged with N₂ three times, and then the mixture was stirred at 25° C. for 3 hours under N₂ atmosphere. On completion, the reaction mixture was diluted with H₂O (40 mL) and extracted with EA (50 mL×3). The combined organic layers were dried over Na₂SO₄, filtered and concentrated under reduced pressure to give a residue. The residue was purified by column chromatography (SiO₂, Petroleum ether/Ethyl acetate=I/O to 19/1) to give the title compound (4.14 g, 92% yield) as a white solid. LC-MS (ESI⁺) m/z 208.1 (M-56)⁺. ¹H NMR (400 MHz, DMSO-d₆) δ=7.38 (dd, J=6.0, 8.5 Hz, 2H), 7.14 (dd, J=2.6, 9.9 Hz, 1H), 7.01 (dt, J=2.8, 8.6 Hz, 1H), 3.97 (br d, J=5.6 Hz, 2H), 2.35 (s, 3H), 1.40 (s, 9H).

Step 2-(S)-3-(4-fluoro-2-methylphenyl)prop-2-yn-1-amine. To a solution of tert-butyl N-[3-(4-fluoro-2-methyl-phenyl)prop-2-ynyl]carbamate (1.5 g, 5.70 mmol) in DCM (15 mL) was added HCl/dioxane (4 M, 7.5 mL). The mixture was stirred at 25° C. for 2 hours. On completion, the reaction mixture was filtered to give the title compound (1.14 g, crude) as a white solid. LC-MS (ESI⁺) m/z 147.2 (M−17). ¹H NMR (400 MHz, DMSO-d₆) δ=8.77 (s, 2H), 7.45 (dd, J=5.9, 8.6 Hz, 1H), 7.18 (dd, J=2.6, 9.9 Hz, 1H), 7.06 (dt, J=2.7, 8.6 Hz, 1H), 3.97 (s, 2H), 3.55 (s, 1H), 3.43 (s, 1H), 2.41 (s, 3H).

Step 3-Tert-butyl ((S)-1-((2S,4R)-2-((3-(4-fluoro-2-methylphenyl)prop-2-yn-1-yl)carbamoyl)-4-hydroxypyrrolidin-1-yl)-3,3-dimethyl-1-oxobutan-2-yl)carbamate. To a solution of (2S,4R)-1-[(2S)-2-(tert-butoxycarbonylamino)-3,3-dimethyl-butanoyl]-4-hydroxy-pyrrolidine-2-carboxylic acid (863 mg, 2.50 mmol, Intermediate EV) in DMF (10 mL) was added HATU (1.24 g, 3.26 mmol) and stirred at 0° C. for 0.5 hour, and then was added DIEA (1.62 g, 12.5 mmol, 2.18 mL) and 3-(4-fluoro-2-methyl-phenyl)prop-2-yn-1-amine (500 mg, 2.50 mmol, HCl). The mixture was stirred at 25° C. for 1.5 hour. On completion, the reaction mixture was diluted with H₂O (40 mL) and extracted with EA (50 mL×3). The combined organic layers were dried over Na₂SO₄, filtered and concentrated under reduced pressure to give a residue. The residue was purified by column chromatography (SiO₂, Petroleum ether/Ethyl acetate=3/1 to 1/1) to give the title compound (1.08 g, 83% yield) as a yellow solid. LC-MS (ESI⁺) m/z 490.3 (M+H)⁺.

Step 4-(2S,4R)-1-((S)-2-amino-3,3-dimethylbutanoyl)-N-(3-(4-fluoro-2-methylphenyl)prop-2-yn-1-yl)-4-hydroxypyrrolidine-2-carboxamide. To a solution of tert-butyl N-[(1S)-1-[(2S,4R)-2-[3-(4-fluoro-2-methyl-phenyl)prop-2-ynylcarbamoyl]-4-hydroxy-pyrrolidine-1-carbonyl]-2,2-dimethyl-propyl]carbamate (1.08 g, 2.20 mmol) in DCM (8 mL) was added HCl/dioxane (4 M, 2 mL). The mixture was stirred at 25° C. for 2 hr. On completion, the reaction mixture was concentrated under reduced pressure to give the title compound (1.14 g, HCl) as a yellow solid. LC-MS (ESI⁺) m/z 390.2 (M+H)⁺.

### (R)-2-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)spiro[3.5]nonane-7-carboxylic acid (Intermediate GZ)

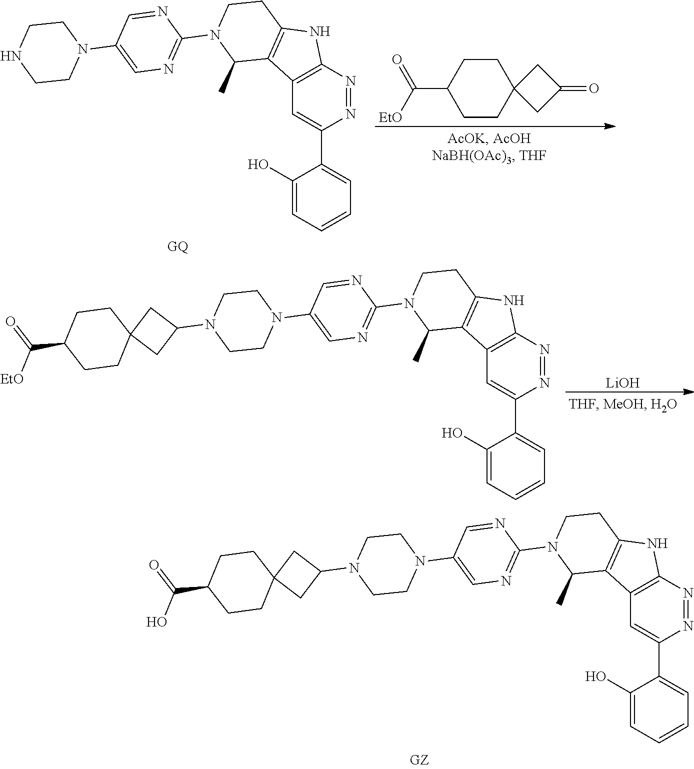

Step 1-(R)-ethyl 2-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)spiro[3.5]nonane-7-carboxylate. To a solution of (R)-2-(5-methyl-6-(5-(piperazin-1-yl)pyrimidin-2-yl)-6,7,8,9-tetrahydro-5H-pyrido[3′,4′:4,5]pyrrolo[2,3-c]pyridazin-3-yl)phenol (500 mg, 1.13 mmol, Intermediate GQ) in DMSO (1 mL) was added AcOK (332.67 mg, 3.39 mmol) and the reaction mixture was stirred at 40° C. for 0.5h. Then to the mixture was added AcOH (203.56 mg, 3.39 mmol) and ethyl 2-oxospiro[3.5]nonane-7-carboxylate (712.75 mg, 3.39 mmol) and stirred at 40° C. for 0.5 h. At last NaBH(OAc)₃ (718.42 mg, 3.39 mmol) was added at 0° C. Then the mixture was stirred at 40° C. for 12 hr. On completion, the reaction mixture was concentrated in vacuo to give a residue. The crude product was purified by column chromatography (SiO₂, Dichloromethane: Methanol=30/1 to 20/1) to give the title compound (525 mg, 50% yield) as a white solid. LC-MS (ESI⁺) m/z 637.4 (M+H)⁺.

Step 2-(R)-2-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)spiro[3.5]nonane-7-carboxylic acid. To a mixture of (R)-ethyl 2-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)spiro[3.5]nonane-7-carboxylate (525 mg, 824.45 umol) in H₂O (2 mL), THF (2 mL) and MeOH (2 mL) was added LiOH (98.72 mg, 4.12 mmol). The mixture was stirred at 25° C. for 3 hours. On completion, the reaction mixture was concentrated in vacuo to give the title compound (500 mg) as a yellow solid. LC-MS (ESI⁺) m/z 609.2 (M+H)⁺.

### 2-(4-(4-Methoxy-4-oxobutyl)phenyl)acetic acid (Intermediate HA)

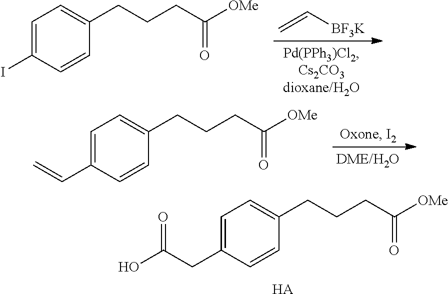

Step 1-Methyl 4-(4-vinylphenyl)butanoate. To a solution of methyl 4-(4-iodophenyl)butanoate (3 g, 11.7 mmol, CAS #756890-85-4) in H₂O (7 mL) and dioxane (30 mL) was added potassium;trifluoro(vinyl)boranuide (2.34 g, 17.5 mmol), Pd(PPh₃)₂Cl₂ (1.23 g, 1.75 mmol), Cs₂CO₃ (7.60 g, 23.3 mmol) and purged with N₂ three times. Then the mixture was stirred at 60° C. for 12 hours under N₂ atmosphere. On completion, the reaction mixture was partitioned between H₂O (40 mL) and ethyl acetate (90 mL). The organic phase was separated, washed with brine (20 mL×3), dried over anhydrous sodium sulfate, filtered and concentrated under reduced pressure to give a residue. The residue was purified by column chromatography (SiO₂, Petroleum ether/Ethyl acetate=15/1) to give the title compound (1.6 g, 60% yield) as a yellow oil. LC-MS (ESI⁺) m/z 205.2 (M+H)⁺.

Step 2-2-(4-(4-methoxy-4-oxobutyl)phenyl)acetic acid. To a solution of methyl 4-(4-vinylphenyl)butanoate (1.6 g, 7.8 mmol) in DME (20 mL) and H₂O (5 mL) was added oxone (2.89 g, 4.70 mmol) and I₂ (199 mg, 783 umol, 158 uL). The mixture was stirred at 25° C. for 8 hours. On completion, the reaction mixture was filtered and concentrated under reduced pressure to give a residue. The crude product was purified by reversed-phase HPLC (0.1% FA condition) to give the title compound (600 mg, 32% yield) as a white solid. ¹H NMR (400 MHz, DMSO-d₆) δ=7.21 (d, J=8.0 Hz, 2H), 7.15 (d, J=8.0 Hz, 2H), 3.67 (s, 3H), 3.63 (s, 2H), 2.64 (t, J=8.0 Hz, 2H), 2.34 (t, J=7.6 Hz, 2H), 2.00-1.90 (m, 2H).

### 4-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)phenyl)butanoic acid (Intermediate HB)

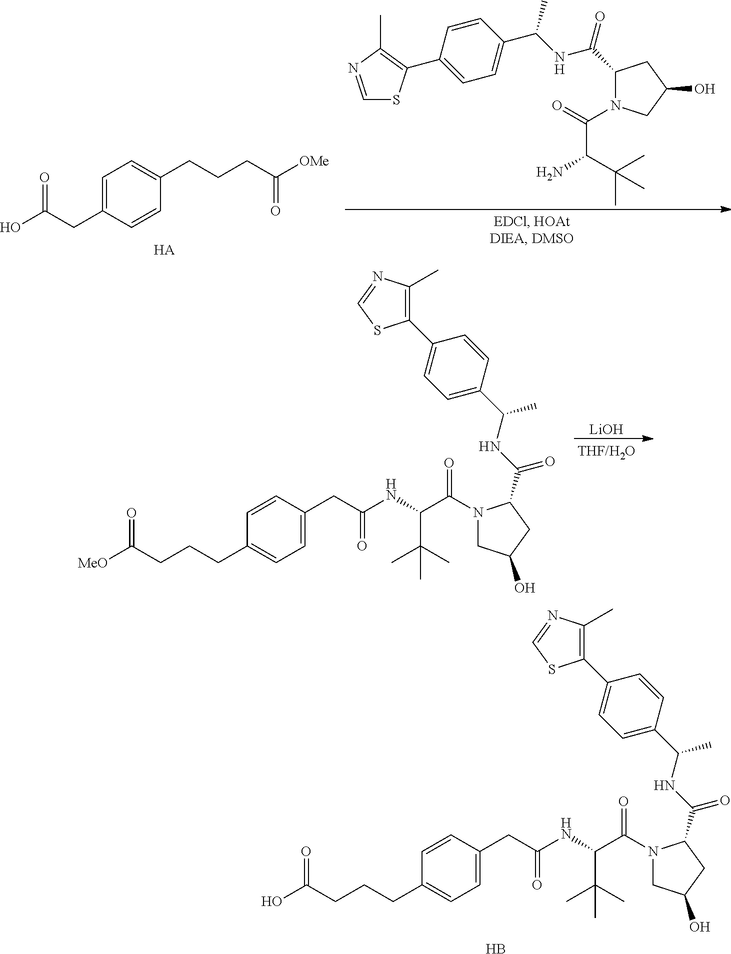

Step 1-Methyl 4-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)phenyl)butanoate. To a solution of 2-(4-(4-methoxy-4-oxobutyl)phenyl)acetic acid (140 mg, 590 umol, Intermediate HA) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (371 mg, 834 umol, HCl salt, CAS #1948273-03-7) in DMSO (4 mL) was added EDCI (227 mg, 1.19 mmol), HOAt (161 mg, 1.19 mmol, 166 uL), and DIEA (383 mg, 2.96 mmol, 516 uL). The mixture was stirred at 25° C. for 2 hours. On completion, the reaction mixture was concentrated in vacuo to give a crude product. The crude product was purified by reversed-phase HPLC (0.1% FA condition) to give the title compound (220 mg, 54% yield) as a white solid. LC-MS (ESI⁺) m/z 663.4(M+H)⁺.

Step 2-4-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)phenyl)butanoic acid. To a solution of methyl 4-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)phenyl)butanoate (220 mg, 332 umol) in THF (3 mL) and H₂O (1 mL) was added LiOH·H₂O (34.8 mg, 830 umol). The mixture was stirred at 25° C. for 6 hr. On completion, the reaction mixture was adjusted pH<5 by adding 1M HCl solution. The solution was then dried by lyophilization to give the title compound (330 mg) as a green solid. LC-MS (ESI⁺) m/z 649.3 (M+H)⁺.

### (S)-(2-(2-hydroxyphenyl)-6a,7,9,10-tetrahydro-5H-pyrazino[1′,2′:4,5]pyrazino[2,3-c]pyridazin-8(6H)-yl)(1-oxa-4,9-diazaspiro[5.5]undecan-4-yl)methanone (Intermediate HC)

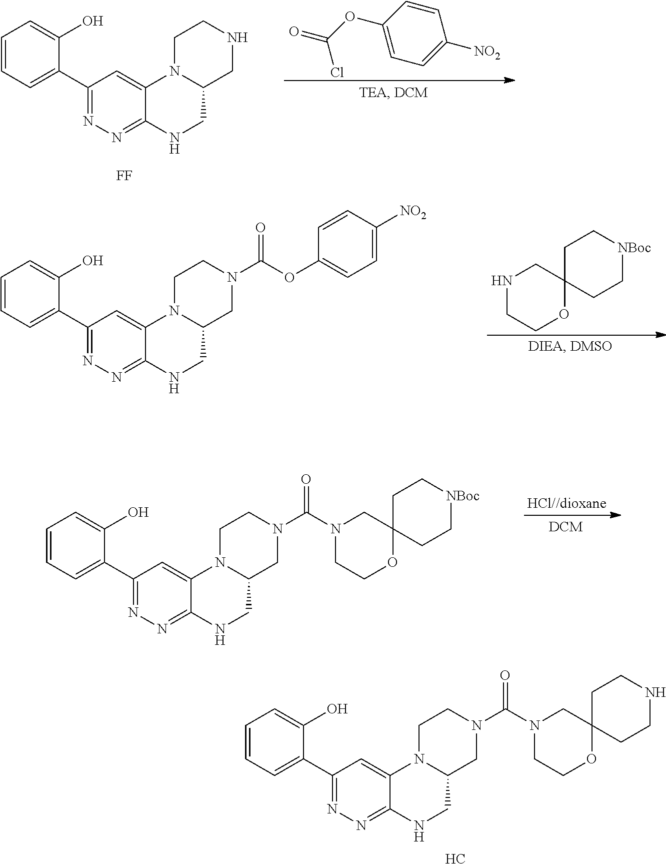

Step 1-(S)-4-nitrophenyl 2-(2-hydroxyphenyl)-6a,7,9,10-tetrahydro-5H-pyrazino[1′,2′:4,5]pyrazino[2,3-c]pyridazine-8(6H)-carboxylate. To a solution of 2-[(10R)-1,5,6,8,12-pentazatricyclo[8.4.0.02,7]tetradeca-2,4,6-trien-4-yl]phenol (412 mg, 1.29 mmol, Intermediate FF) in DCM (6 mL) was added 4-nitrophenyl carbonochloridate (207 mg, 1.03 mmol) and TEA (391 mg, 3.87 mmol) at 0° C. The reaction solution was stirred at 0° C. for 1 hr. On completion, the reaction mixture was concentrated in vacuo to give the title compound (628 mg) as a deep yellow solid. LC-MS (ESI⁺) m z 449.0 (M+H)⁺.

Step 2-(S)-tert-butyl 4-(2-(2-hydroxyphenyl)-6,6a,7,8,9,10-hexahydro-5H-pyrazino[1′,2′:4,5]pyrazino[2,3-c]pyridazine-8-carbonyl)-1-oxa-4,9-diazaspiro[5.5]undecane-9-carboxylate. To a mixture (S)-4-nitrophenyl 2-(2-hydroxyphenyl)-6a,7,9,10-tetrahydro-5H-pyrazino[1′,2′:4,5]pyrazino[2,3-c]pyridazine-8(6H)-carboxylate (578 mg, 1.29 mmol) in dioxane (10 mL) and DMSO (1 mL) was added DIEA (666 mg, 5.16 mmol) and tert-butyl 1-oxa-4,9-diazaspiro[5.5]undecane-9-carboxylate (1.16 g, 4.51 mmol, CAS #930785-40-3) at 25° C. The mixture was stirred at 100° C. for 12 hours. On completion, the reaction mixture was concentrated in vacuo to give a residue. The crude product was purified by reversed-phase HPLC (0.1% TFA condition) to give the title compound (142 mg, TFA) as yellow solid. LC-MS (ESI⁺) m/z 566.3 (M+H)⁺.

Step 3-(S)-(2-(2-hydroxyphenyl)-6a,7,9,10-tetrahydro-5H-pyrazino[1′,2′:4,5]pyrazino[2,3-c]pyridazin-8(6H)-yl)(1-oxa-4,9-diazaspiro[5.5]undecan-4-yl)methanone. To a solution of (S)-tert-butyl 4-(2-(2-hydroxyphenyl)-6,6a,7,8,9,10-hexahydro-5H-pyrazino[1′,2′:4,5]pyrazino[2,3-c]pyridazine-8-carbonyl)-1-oxa-4,9-diazaspiro[5.5]undecane-9-carboxylate (142 mg, 208 umol) in DCM (2 mL) was added HCl/dioxane (4 M, 0.1 mL). The mixture was stirred at 25° C. for 1 hour. On completion, the reaction mixture was concentrated in vacuo to give the title compound (164 mg) as a white solid. LC-MS (ESI⁺) m/z 466.2 (M+H)⁺.

### (2S,4R)-1-((S)-3,3-dimethyl-2-(2-oxospiro[3.5]nonane-7-carboxamido)butanoyl)-4-hydroxy-N-(4-(4-methylthiazol-5-yl)benzyl)pyrrolidine-2-carboxamide (Intermediate HD)

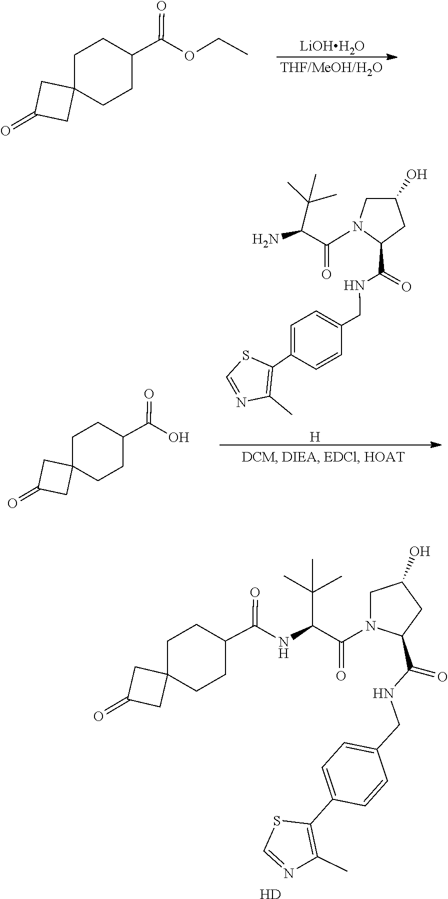

Step 1-2-Oxospiro[3.5]nonane-7-carboxylic acid. To a solution of ethyl 2-oxospiro[3.5]nonane-7-carboxylate (300 mg, 1.43 mmol) in THF (2 mL), MeOH (2 mL) and H₂O (2 mL) was added LiOH·H₂O (239 mg, 5.71 mmol). The mixture was stirred at 25° C. for 2 hours. On completion, the pH of the mixture was adjusted to 3-4 with 1 M HCl. The residue extracted with DCM (30 mL×5). The combined organic layers were washed with brine (20 mL×3), dried over Na₂SO₄ filtered and concentrated under reduced pressure to give the title compound (233 mg) as white solid.

Step 2-(2S,4R)-1-((S)-3,3-dimethyl-2-(2-oxospiro[3.5]nonane-7-carboxamido)butanoyl)-4-hydroxy-N-(4-(4-methylthiazol-5-yl)benzyl)pyrrolidine-2-carboxamide. To a solution of 2-oxospiro[3.5]nonane-7-carboxylic acid (233 mg, 1.28 mmol) and (2S,4R)-1-((S)-2-amino-3,3-dimethylbutanoyl)-4-hydroxy-N-(4-(4-methylthiazol-5-yl)benzyl)pyrrolidine-2-carboxamide (605 mg, 1.41 mmol, Intermediate H) in DCM (8 mL) was added HOAt (261 mg, 1.92 mmol), EDCI (367 mg, 1.92 mmol) and DIEA (661 mg, 5.11 mmol). The mixture was stirred at 25° C. for 1 hour. On completion, the residue was diluted with H₂O (30 mL) and extracted with DCM (30 mL×4). The combined organic layers were washed with brine (20 mL×2), dried over Na₂SO₄, and concentrated in vacuo to give a residue. The residue was purified by column chromatography (SiO₂, Petroleum ether/Ethyl acetate=1/1 to DCM: MeOH=25:1) to give the title compound (627 mg, 78% yield) as a white solid. LC-MS (ESI⁺) m/z 595.1 (M+H)⁺.

### (2S,4R)-1-((S)-2-amino-3,3-dimethylbutanoyl)-4-hydroxy-N-(4-(prop-1-yn-1-yl)benzyl)pyrrolidine-2-carboxamide (Intermediate HE)

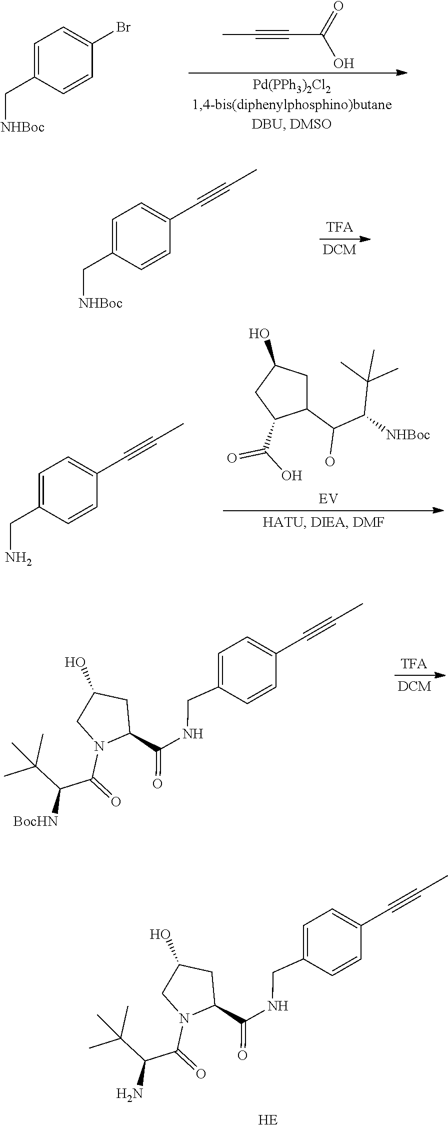

Step 1-Tert-butyl 4-(prop-1-yn-1-yl)benzylcarbamate. A mixture of but-2-ynoic acid (2.64 g, 31.4 mmol), tert-butyl 4-bromobenzylcarbamate (3 g, 10.5 mmol), DBU (4.79 g, 31.5 mmol, 4.74 mL), Pd(PPh₃)₂Cl₂ (736 mg, 1.05 mmol) and 1,4-bis(diphenylphosphino)butane (894 mg, 2.10 mmol) in DMSO (50 mL) was degassed and purged with N₂ three times. Then the mixture was stirred at 110° C. for 12 hours under N₂ atmosphere. On completion, the reaction mixture was diluted with H₂O (100 mL) and extracted with EA (3×100 mL). The combined organic layers were washed with brine, dried over Na₂SO₄, filtered and concentrated under reduced pressure to give a residue. The residue was purified by column chromatography (SiO₂, Petroleum ether/Ethyl acetate=I/O to 10/1) to give the title compound (1.4 g, 33% yield) as yellow oil. ¹H NMR (400 MHz, DMSO-d₆) δ=7.33 (d, J=8.0 Hz, 2H), 7.20 (d, J=8.0 Hz, 2H), 4.12 (d, J=6.0 Hz, 2H), 2.03 (s, 3H), 1.40 (s, 9H). LC-MS (ESI⁺) m/z 190.0 (M-55)⁺.

Step 2-Tert-butyl ((S)-1-((2S,4R)-4-hydroxy-2-((4-(prop-1-yn-1-yl)benzyl)carbamoyl)pyrrolidin-1-yl)-3,3-dimethyl-1-oxobutan-2-yl)carbamate. To a solution of tert-butyl 4-(prop-1-yn-1-yl)benzylcarbamate (700 mg, 2.85 mmol) in DCM (10 mL) was added TFA (1.63 g, 14.2 mmol, 1.06 mL). The mixture was stirred at 25° C. for 2 hours. On completion, the reaction mixture was filtered and concentrated under reduced pressure to give the title compound (353 mg) as a yellow solid. ¹H NMR (400 MHz, DMSO-d₆) δ=7.43 (d, J=1.2 Hz, 4H), 4.04 (s, 2H), 2.05 (s, 3H).

Step 3-(4-Ethynylphenyl)methanamine. To a solution of (4-(prop-1-yn-1-yl)phenyl)methanamine (350 mg, 2.41 mmol) in DMF (4 mL) was added HATU (1.10 g, 2.89 mmol), DIEA (1.56 g, 12.1 mmol, 2.10 mL) and (2S,4R)-1-((S)-2-((tert-butoxycarbonyl)amino)-3,3-dimethylbutanoyl)-4-hydroxypyrrolidine-2-carboxylic acid (996 mg, 2.89 mmol, Intermediate EV). The mixture was stirred at 25° C. for 2 hours. On completion, the reaction mixture was filtered and concentrated under reduced pressure to give a residue. The residue was purified by prep-HPLC (0.1% FA) to give the title compound (725 mg, 57% yield) as a white solid. LC-MS (ESI⁺) m/z 472.2 (M+H)⁺.

Step 4-(2S,4R)-1-((S)-2-amino-3,3-dimethylbutanoyl)-4-hydroxy-N-(4-(prop-1-yn-1-yl)benzyl)pyrrolidine-2-carboxamide. A mixture of tert-butyl ((S)-1-((2S,4R)-4-hydroxy-2-((4-(prop-1-yn-1-yl)benzyl)carbamoyl)pyrrolidin-1-yl)-3,3-dimethyl-1-oxobutan-2-yl)carbamate (724 mg, 1.54 mmol), and TFA (770 mg, 6.75 mmol, 0.5 mL) in DCM (5 mL) was degassed and purged with N₂ three times. Then the mixture was stirred at 25° C. for 1 hour under N₂ atmosphere. On completion, the reaction mixture was filtered and concentrated under reduced pressure to give the title compound (1 g) as a yellow solid. LC-MS (ESI⁺) m/z 372.1 (M+H)⁺.

### (2S,4R)-1-((S)-2-amino-3,3-dimethylbutanoyl)-N-(3-(4-chloro-2-methylphenyl)prop-2-yn-1-yl)-4-hydroxypyrrolidine-2-carboxamide (Intermediate HF)

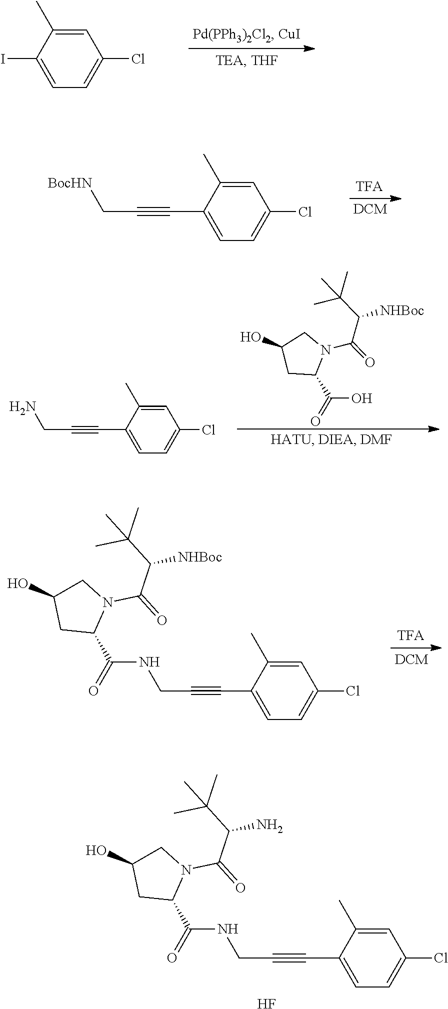

Step 1-Tert-butyl (3-(4-chloro-2-methylphenyl)prop-2-yn-1-yl)carbamate. A mixture of 4-chloro-1-iodo-2-methylbenzene (5 g, 19.8 mmol, CAS #23399-70-4), tert-butyl N-prop-2-ynylcarbamate (6.15 g, 39.6 mmol), CuI (754 mg, 3.96 mmol), TEA (6.01 g, 59.4 mmol) and dichloropalladium;triphenylphosphane (1.39 g, 1.98 mmol), in THF (125 mL) was degassed and purged with N₂ three times. Then the mixture was stirred at 25° C. for 2 hours under N₂ atmospher. The reaction mixture was partitioned between brine (60 mL) and ethyl acetate (60 mL). The organic phase was separated, washed with brine (60 mL), dried over Na₂SO₄, filtered and concentrated under reduced pressure to give a residue. Then the residue was purified by column chromatography (SiO₂, Petroleum ether/Ethyl acetate=I/O to 5/1) to give the title compound (4 g, 69% yield) as a white solid. LC-MS (ESI⁺) m/z 224.0 (M−C₄H₉)⁺.

Step 2-3-(4-Chloro-2-methylphenyl)prop-2-yn-1-amine. To a solution of tert-butyl (3-(4-chloro-2-methylphenyl)prop-2-yn-1-yl)carbamate (200 mg, 715 umol) in DCM (4 mL) was added TFA (616 mg, 5.40 mmol). The mixture was stirred at 25° C. for 1 hr. On the completion, the reaction mixture was concentrated under reduced pressure to give the title compound (128 mg) as a white solid.

Step 3-Tert-butyl ((S)-1-((2S,4R)-2-((3-(4-chloro-2-methylphenyl)prop-2-yn-1-yl)carbamoyl)-4-hydroxypyrrolidin-1-yl)-3,3-dimethyl-1-oxobutan-2-yl)carbamate. To a solution of (2S,4R)-1-((S)-2-((tert-butoxycarbonyl)amino)-3,3-dimethylbutanoyl)-4-hydroxypyrrolidine-2-carboxylic acid (271 mg, 788 umol, CAS #630421-46-4) in DMF (3.6 mL) was added HATU (300 mg, 788 umol), 3-(4-chloro-2-methylphenyl)prop-2-yn-1-amine (118 mg, 657 umol) and DIEA (1.02 g, 7.88 mmol, 1.37 mL). The mixture was stirred at 25° C. for 2 hours. On the completion, the reaction mixture was partitioned between brine (20 mL) and ethyl acetate (30 mL). The organic phase was separated, washed with brine (30 mL), dried over Na₂SO₄, filtered and concentrated under reduced pressure to give a residue. Then the residue was purified by column chromatography (SiO₂, Petroleum ether/Ethyl acetate=I/O to 1/1) to give the title compound (400 mg) as a white solid. LC-MS (ESI⁺) m/z 506.2 (M+H)⁺.

Step 4-(2S,4R)-1-((S)-2-amino-3,3-dimethylbutanoyl)-N-(3-(4-chloro-2-methylphenyl)prop-2-yn-1-yl)-4-hydroxypyrrolidine-2-carboxamide. To a solution of tert-butyl N-[(1S)-1-[(2S,4R)-2-[3-(4-chloro-2-methyl-phenyl)prop-2-ynylcarbamoyl]-4-hydroxy-pyrrolidine-1-carbonyl]-2,2-dimethyl-propyl]carbamate (300 mg, 593 umol) was added in TFA (1 mL) and DCM (5 mL). The mixture was stirred at 25° C. for 0.5 hour. The reaction mixture was filtered and concentrated under reduced pressure to give a residue. The crude product was purified by reversed-phase HPLC(0.1% FA condition) to give the title compound (160 mg, 56% yield) as a yellow solid. LC-MS (ESI⁺) m/z 406.2 (M+H)⁺.

### (4-Ethynylphenyl)methanamine (Intermediate HG)

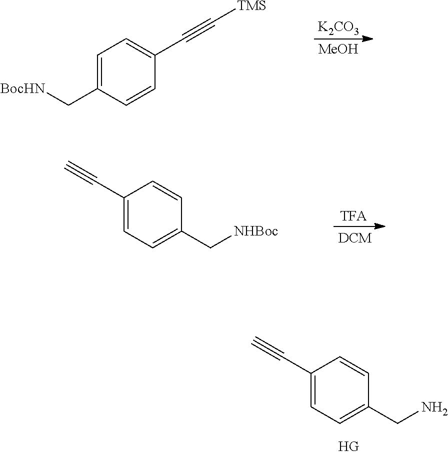

Step 1-Tert-butyl N-[(4-ethynylphenyl)methyl]carbamate. To a solution of tert-butyl N-[[4-(2-trimethylsilylethynyl)phenyl]methyl]carbamate (4.4 g, 14.5 mmol, CAS #680190-95-8) in MeOH (55 mL) was added K₂CO₃ (4.01 g, 29.0 mmol). The mixture was stirred at 25° C. for 2 hours. The reaction mixture was partitioned between EA (300 mL) and water (100 mL). The organic phase was separated, washed with brine (50 mL×2), dried over Na₂SO₄, filtered and concentrated under reduced pressure to give the title compound (3.3 g) as a yellow solid. LC-MS (ESI⁺) m/z 176.2 (M-55)⁺.

Step 2-(4-Ethynylphenyl)methanamine. To a solution of tert-butyl N-[(4-ethynylphenyl)methyl]carbamate (1.5 g, 6.49 mmol) in DCM (10 mL) was added TFA (2.96 g, 25.9 mmol). The mixture was stirred at 25° C. for 0.5 hour. On completion, the reaction mixture was filtered and concentrated under reduced pressure to give the title compound (837 mg) as obtained as a yellow oil. LC-MS (ESI⁺) m/z 115.2 (M-NH₂)⁺.

### (2S,4R)-1-[(2S)-2-amino-3,3-dimethyl-butanoyl]-N-[(4-ethynylphenyl)methyl]-4-hydroxy-pyrrolidine-2-carboxamide (Intermediate HH)

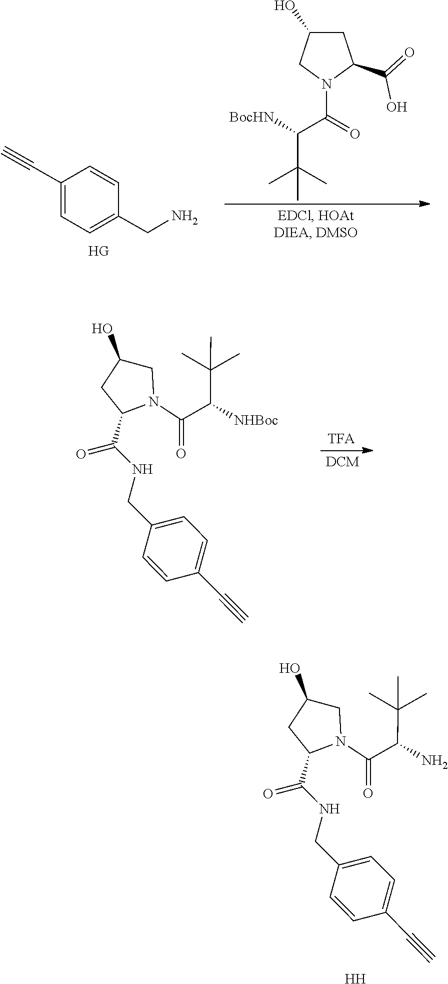

Step 1-Tert-butyl N-[(1S)-1-[(2S,4R)-2-[(4-ethynylphenyl)methylcarbamoyl]-4-hydroxy-pyrrolidine-1-carbonyl]-2,2-dimethyl-propyl]carbamate. To a solution of (4-ethynylphenyl)methanamine (800 mg, 6.1 mmol, Intermediate HG) and (2S,4R)-1-[(2S)-2-(tert-butoxycarbonylamino)-3,3-dimethyl-butanoyl]-4-hydroxy-pyrrolidine-2-carboxylic acid (2.1 g, 6.1 mmol, CAS #630421-46-4) in DMSO (10 mL) was added EDCI (1.75 g, 9.15 mmol) and HOAt (1.24 g, 9.15 mmol) and DIEA (3.94 g, 30.5 mmol). The mixture was stirred at 25° C. for 12 hours. On completion, the reaction mixture was purified directly. The crude product was purified by reversed-phase HPLC (0.1% FA condition) to give the title compound (2.3 g, 77% yield) as a white solid. LC-MS (ESI⁺) m/z 458.2 (M+H)⁺.

Step 2-(2S,4R)-1-[(2S)-2-amino-3,3-dimethyl-butanoyl]-N-[(4-ethynylphenyl)methyl]-4-hydroxy-pyrrolidine-2-carboxamide. To a solution of tert-butyl N-[(1S)-1-[(2S,4R)-2-[(4-ethynylphenyl)methylcarbamoyl]-4-hydroxy-pyrrolidine-1-carbonyl]-2,2-dimethyl-propyl]carbamate (1 g, 2 mmol) was added in DCM (10 mL) and TFA (2 mL). The mixture was stirred at 25° C. for 0.5 hour. On completion, the reaction mixture was filtered and concentrated under reduced pressure to give a residue. The crude product was purified by reversed-phase HPLC (0.1% FA condition) to give the title compound (620 mg, 64% yield) as a yellow solid. LC-MS (ESI⁺) m/z 358.2 (M+H)⁺.

### Phenyl ((S)-1-((2S,4R)-2-((4-ethynylbenzyl)carbamoyl)-4-hydroxypyrrolidin-1-yl)-3,3-dimethyl-1-oxobutan-2-yl)carbamate (Intermediate HI)

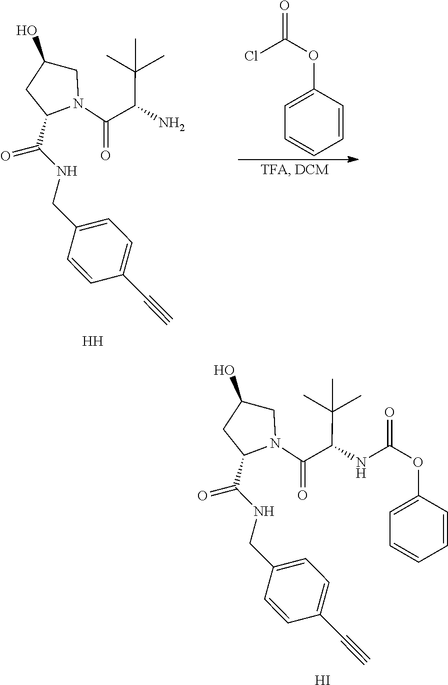

Step 1-Phenyl ((S)-1-((2S,4R)-2-((4-ethynylbenzyl)carbamoyl)-4-hydroxypyrrolidin-1-yl)-3,3-dimethyl-1-oxobutan-2-yl)carbamate. To a solution of (2S,4R)-1-[(2S)-2-amino-3,3-dimethyl-butanoyl]-N-[(4-ethynylphenyl)methyl]-4-hydroxy-pyrrolidine-2-carboxamide (500 mg, 1.40 mmol, Intermediate HH) and phenyl carbonochloridate (438 mg, 2.80 mmol, CAS #1885-14-9) in DCM (5 mL) was added TEA (849 mg, 8.39 mmol). The mixture was stirred at −10° C. for 1 hour. The reaction mixture was partitioned between DCM (50 mL) and water (40 mL). The organic phase was separated, dried over Na₂SO₄, filtered and concentrated under reduced pressure to give a residue. The residue was purified by column chromatography (SiO₂, Petroleum ether/Ethyl acetate=10/1 to DCM/MeOH=10/1) to give the title compound (220 mg, 20% yield) as a white solid. LC-MS (ESI⁺) m/z 478.2 (M+H)⁺.

### (2S,4R)-1-((S)-2-benzamido-3,3-dimethylbutanoyl)-N-(4-ethynylbenzyl)-4-hydroxypyrrolidine-2-carboxamide (Intermediate HJ)

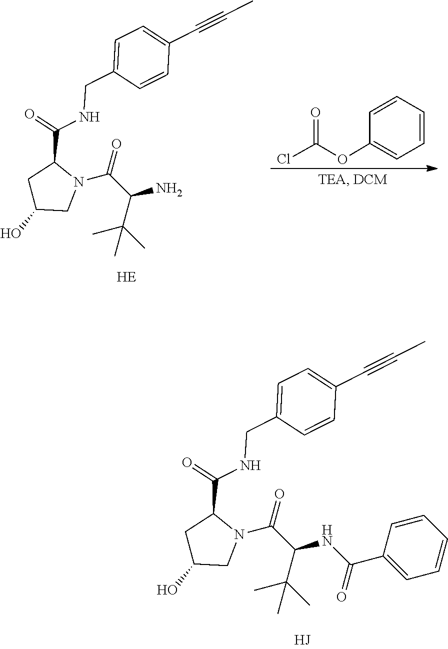 [SMILES: CC#Cc1ccc(CNC(=O)[C@@H]2C[C@@H](O)CN2C(=O)[C@@H](N)C(C)(C)C)cc1 ; O=C(Cl)Oc1ccccc1 ; CC#Cc1ccc(CNC(=O)[C@@H]2C[C@@H](O)CN2C(=O)[C@@H](NC(=O)c2ccccc2)C(C)(C)C)cc1]

To a solution of (2S,4R)-1-((S)-2-amino-3,3-dimethylbutanoyl)-N-(4-ethynylbenzyl)-4-hydroxypyrrolidine-2-carboxamide (300 mg, 800 umol, Intermediate HE) in DCM (3 mL) was added TEA (245.16 mg, 2.42 mmol, 337.22 uL) at 25° C. Then phenyl carbonochloridate (252.88 mg, 1.62 mmol) was added at 0° C. The reaction solution was stirred at 25° C. for 2 hours. On completion, the reaction mixture was concentrated in vacuo to give a residue. The crude product was purified by column chromatography (SiO₂, Petroleum ether/Ethyl acetate=6/1 to 1/1) to give the title compound (263 mg, 44% yield) as a white solid. LC-MS (ESI⁺) m/z 492.2 (M+H)⁺.

### Methyl 6-(4-oxopiperidin-1-yl)spiro[3.3]heptane-2-carboxylate (Intermediate HK)

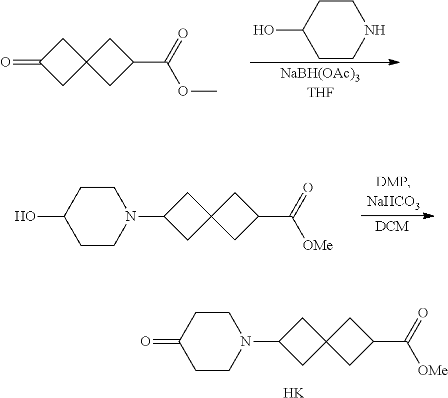

Step 1-Methyl 6-(4-hydroxypiperidin-1-yl)spiro[3.3]heptane-2-carboxylate. To a solution of piperidin-4-ol (1.5 g, 14.8 mmol) and methyl 2-oxospiro[3.3]heptane-6-carboxylate (2.49 g, 14.8 mmol) in THF (20 mL) was added NaBH(OAc)₃ (6.29 g, 29.6 mmol). Then the mixture was stirred at 25° C. for 12 hours. On completion, the reaction mixture was quenched with MeOH (10 mL) and concentrated to give a residue. The residue was purified by column chromatography (SiO₂, Petroleum ether/Ethyl acetate=1/1 to 0/1) to afford the title compound (3.7 g, 98% yield) as a colorless oil. LC-MS (ESI⁺) m z 254.2 (M+H)⁺.

Step 2-Methyl 6-(4-oxopiperidin-1-yl)spiro[3.3]heptane-2-carboxylate. To a solution of methyl 2-(4-hydroxy-1-piperidyl)spiro[3.3]heptane-6-carboxylate (1 g, 3.95 mmol) in DCM (25 mL) was added DMP (2.51 g, 5.92 mmol) and NaHCO₃ (2.34 g, 27.8 mmol) and 4 Å molecular sieves (2 g) at 0° C. Then the mixture was stirred at 0-25° C. for 1 hour. The reaction mixture was quenched with Na₂SO₃ (20 mL) and extracted with DCM (20 mL). The organic layers were dried over anhydrous Na₂SO₄, filtered and concentrated. The residue was purified by column chromatography (SiO₂, Petroleum ether/Ethyl acetate=1/1 to 0/1) to afford the title compound (200 mg, 20% yield) as a yellow oil. ¹H NMR (400 MHz, CDCl₃-d) δ ppm: 3.60 (s, 3H), 3.00-2.98 (s, 1H), 2.66 (m, 1H) 2.53 (m, 4H) 2.50 (m, 4H) 2.39-2.35 (m, 4H), 2.23-2.18 (m, 2H), 1.89-1.86 (m, 2H).

### (S)-6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylic acid (Intermediate HL)

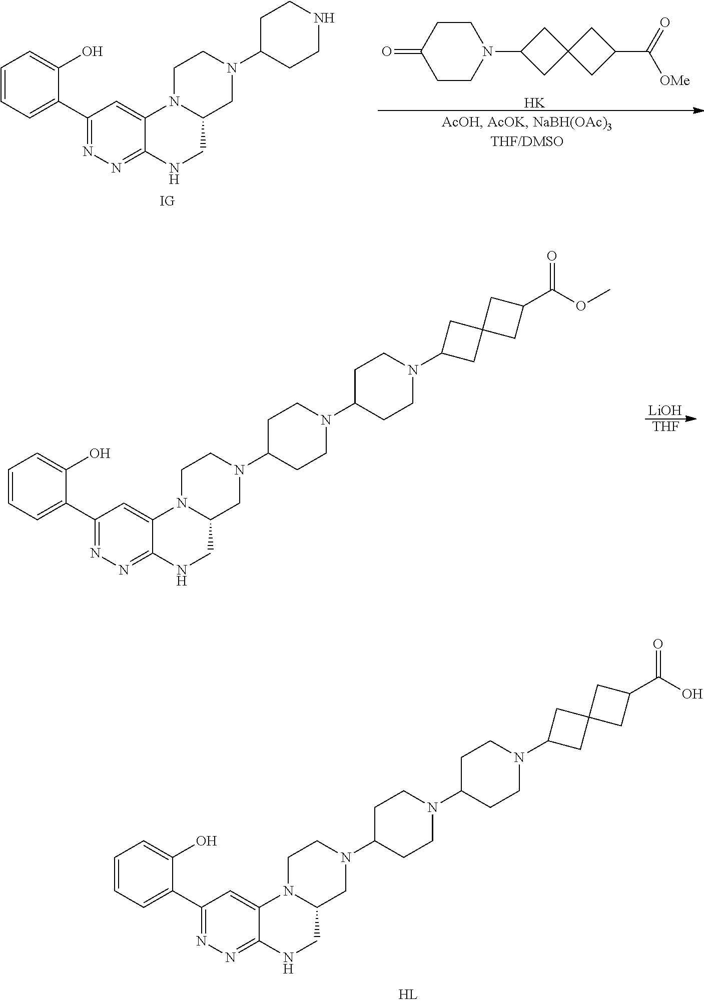

Step 1-(S)-methyl 6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylate. To a solution of 2-[(10S)-12-(4-piperidyl)-1,5,6,8,12-pentazatricyclo[8.4.0.02,7]tetradeca-2,4,6-trien-4-yl]phenol (420 mg, 1.15 mmol, Intermediate IG) and methyl 2-(4-oxo-1-piperidyl)spiro[3.3]heptane-6-carboxylate (432 mg, 1.72 mmol, Intermediate HK) in THF (4 mL) and DMSO (4 mL) was added KOAc (224 mg, 2.29 mmol) and HOAc (206 mg, 3.44 mmol). The mixture was stirred for 0.5 hour. Then NaBH(OAc)₃ (485 mg, 2.29 mmol) was added and the mixture was stirred at 25° C. for 12 hr. The reaction mixture was quenched with MeOH (1 mL) and purified directly. The solution was purified by reversed phase flash (FA) to give the title compound (250 mg, 36% yield) as a yellow solid. LC-MS (ESI+) m/z 602.4 (M+H)⁺.

Step 2-(S)-6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylic acid. To a solution of methyl 2-[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]spiro[3.3]heptane-6-carboxylate (170 mg, 282 umol) in THF (1 mL) was added LiOH·H₂O (85.6 mg, 2 M). The mixture was stirred at 25° C. for 1 hour. On completion, the reaction mixture was filtered and concentrated under reduced pressure to give a residue. The crude product was purified by reversed-phase HPLC(0.1% FA condition) to give the title compound (126 mg, 70% yield) as a white solid. LC-MS (ESI+) m/z 588.5 (M+H)⁺.

### (2-Chloro-4-ethynylphenyl)methanamine (Intermediate HM)

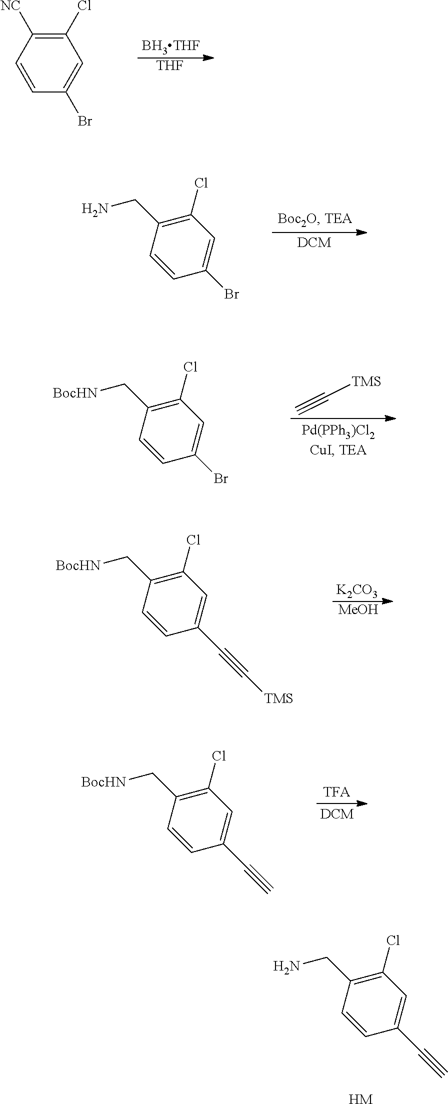

Step 1-(4-Bromo-2-chlorophenyl)methanamine. To a solution of 4-bromo-2-chloro-benzonitrile (2.7 g, 13 mmol, CAS #154607-01-9) in THF (20 mL) was added BH₃.THF (1 M, 43.6 mL) at 0° C. The mixture was stirred at 80° C. for 2 hours. On completion, the reaction mixture was quenched with 1M HCl until the pH=4, and then NaOH aqueous solution was added until the pH=10 and extracted with DCM (100 mL×3). The combined organic layers were washed with brine (50 mL×2), dried over Na₂SO₄, filtered and concentrated under reduced pressure to give the title compound (3 g) as a white solid. LC-MS (ESI⁺) m/z 204.9 (M−16)⁺.

Step 2-Tert-butyl 4-bromo-2-chlorobenzylcarbamate. To a solution of (4-bromo-2-chloro-phenyl)methanamine (3 g, 13.6 mmol) in DCM (80 mL) was added Boc₂O (3.27 g, 14.9 mmol) and TEA (4.13 g, 40.8 mmol). The mixture was stirred at 25° C. for 1 hour. On completion, the reaction mixture was quenched with H₂O (30 mL) and extracted with DCM (70 mL×3). The combined organic layers were washed with brine (60 mL), dried over Na₂SO₄, filtered and concentrated under reduced pressure to give the title compound (4.0 g) as a white solid. LC-MS (ESI⁺) m/z 266.0 (M−55)⁺.

Step 3-Tert-butyl 2-chloro-4-((trimethylsilyl)ethynyl)benzylcarbamate. To a solution of tert-butyl N-[(4-bromo-2-chloro-phenyl)methyl]carbamate (3.8 g, 12 mmol), ethynyl(trimethyl)silane (11.6 g, 118 mmol) in TEA (50 mL) was added Pd(PPh₃)₂Cl₂ (831 mg, 1.19 mmol) and CuI (451 mg, 2.37 mmol). The mixture was stirred at 80° C. for 12 hours under N₂ atmosphere. On completion, the reaction mixture was filtered and concentrated under reduced pressure to give a residue. The residue was purified by column chromatography (SiO₂, Petroleum ether/Ethyl acetate=100/1 to 50/1) to give the title compound (2.48 g, 50% yield) as a brown oil. LC-MS (ESI⁺) m/z 282.0 (M−55)⁺.

Step 4-Tert-butyl 2-chloro-4-ethynylbenzylcarbamate. To a solution of tert-butyl N-[[2-chloro-4-(2-trimethylsilylethynyl)phenyl]methyl]carbamate (2.48 g, 7.34 mmol) in MeOH (30 mL) was added K₂CO₃ (2.03 g, 14.7 mmol). The mixture was stirred at 25° C. for 2 hours. On completion, the reaction mixture was quenched by addition of H₂O (30 mL) and extracted with EA (30 mL×3). The combined organic layers were dried over NaSO₄, filtered and concentrated under reduced pressure to give the title compound (1.47 g) as a brown oil. LC-MS (ESI⁺) m/z 210.0 (M−55)⁺.

Step 5-(2-chloro-4-ethynylphenyl)methanamine. To a solution of tert-butyl N-[(2-chloro-4-ethynyl-phenyl)methyl]carbamate (1.2 g, 4.5 mmol) in DCM (30 mL) was added TFA (10 mL). The mixture was stirred at 25° C. for 0.5 hours. On completion, the reaction mixture was filtered and concentrated under reduced pressure to give the title compound (2.4 g, TFA) as a brown oil. LC-MS (ESI⁺) m/z 166.2 (M+H)⁺.

### (2S,4R)-1-((S)-2-Amino-3,3-dimethylbutanoyl)-N-(2-chloro-4-ethynylbenzyl)-4-hydroxypyrrolidine-2-carboxamide (Intermediate HN)

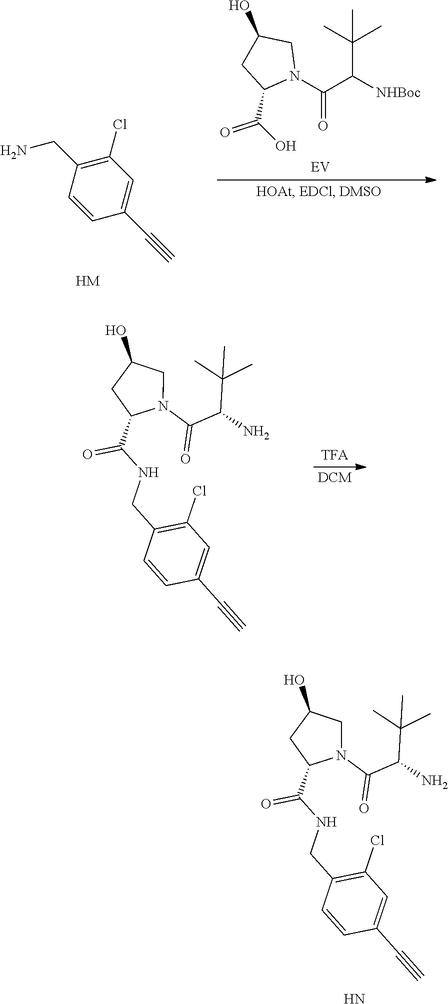

Step 1-Tert-butyl ((S)-1-((2S,4R)-2-((2-chloro-4-ethynylbenzyl)carbamoyl)-4-hydroxypyrrolidin-1-yl)-3,3-dimethyl-1-oxobutan-2-yl)carbamate. To a solution of (2-chloro-4-ethynyl-phenyl)methanamine (2 g, 7.15 mmol, TFA, Intermediate HM), (2S,4R)-1-[2-(tert-butoxycarbonylamino)-3,3-dimethyl-butanoyl]-4-hydroxy-pyrrolidine-2-carboxylic acid (1.97 g, 5.72 mmol, Intermediate EV) in DMSO (20 mL) was added DIEA (2.77 g, 21.4 mmol, 3.74 mL), EDCI (2.06 g, 10.7 mmol) and HOAt (1.46 g, 10.7 mmol). The mixture was stirred at 25° C. for 2 hrs. On completion, the reaction mixture was diluted with H₂O (10 mL) and extracted with EA (70 mL×3). The combined organic layers were washed with brine (30 mL×2), dried over Na₂SO₄, filtered and concentrated under reduced pressure to give a residue. The residue was purified by column chromatography (SiO₂, Petroleum ether/Ethyl acetate=5/1 to 1/1) to give the title compound (2.5 g, 57% yield) as a yellow oil. LC-MS (ESI⁺) m/z 492.0 (M+H)⁺.

Step 2-(2S,4R)-1-((S)-2-amino-3,3-dimethylbutanoyl)-N-(2-chloro-4-ethynylbenzyl)-4-hydroxypyrrolidine-2-carboxamide. To a solution of tert-butyl N-[(1S)-1-[(2S,4R)-2-[(2-chloro-4-ethynyl-phenyl)methylcarbamoyl]-4-hydroxy-pyrrolidine-1-carbonyl]-2,2-dimethyl-propyl]carbamate (1 g, 2 mmol) in DCM (10 mL) was added TFA (2 mL). The mixture was stirred at 25° C. for 1 hour. On completion, the reaction mixture was filtered and concentrated under reduced pressure to give a residue. The residue was purified by prep-HPLC (FA condition) to give the title compound (550 mg, 59% yield, FA) as a yellow oil. LC-MS (ESI⁺) m/z 392.0 (M+H)⁺.

### (4-Ethynyl-2-methoxyphenyl)methanamine (Intermediate HO)

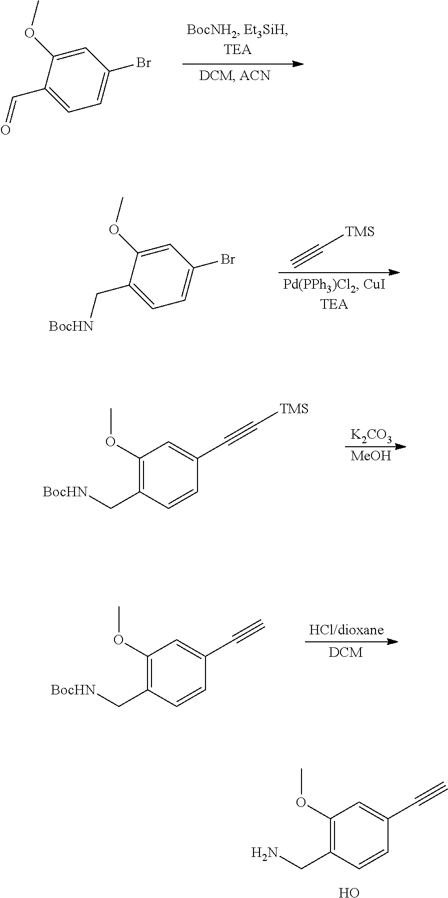

Step 1-Tert-butyl 4-bromo-2-methoxybenzylcarbamate. To a solution of 4-bromo-2-methoxy-benzaldehyde (5 g, 23.2 mmol, CAS #43192-33-2) and tert-butyl carbamate (8.17 g, 69.8 mmol) in CH₂Cl₂ (50 mL) and MeCN (150 mL) was added Et₃SiH (8.11 g, 69.8 mmol, 11.1 mL) and TFA (5.30 g, 46.5 mmol, 3.44 mL). The mixture was stirred at 20° C. for 18 hours. On completion, the reaction mixture was partitioned between NaHCO₃ (30 mL) and CH₂Cl₂ (50 mL). The organic phase was separated, dried over anhydrous sodium sulfate, filtered and concentrated under reduced pressure to give a residue. The residue was purified by column chromatography (SiO₂, Petroleum ether/Ethyl acetate=10/1) to give the title compound (6.6 g, 90% yield) as a white solid. LC-MS (ESI⁺) m/z 340.0 (M+Na)⁺.

Step 2-Tert-butyl 2-methoxy-4-((trimethylsilyl)ethynyl)benzylcarbamate. To a solution of tert-butyl N-[(4-bromo-2-methoxy-phenyl)methyl]carbamate (4 g, 13 mmol) in TEA (50 mL) was added ethynyl(trimethyl)silane (12.4 g, 127 mmol, 17.5 mL), CuI (482 mg, 2.53 mmol), Pd(PPh₃)₂Cl₂ (888 mg, 1.27 mmol) and purged with N₂ three times. Then the mixture was stirred at 80° C. for 12 hours under N₂ atmosphere. On completion, the reaction mixture was concentrated in vacuo to give a residue. The residue was purified by column chromatography (SiO₂, Petroleum ether/Ethyl acetate=15/1) to give the title compound (2.9 g 59% yield) as a brown solid. LC-MS (ESI⁺) m/z 356.3 (M+Na)⁺.

Step 3-Tert-butyl 4-ethynyl-2-methoxybenzylcarbamate. To a solution of tert-butyl N-[[2-methoxy-4-(2-trimethylsilylethynyl)phenyl]methyl]carbamate (2.9 g, 8.7 mmol) in MeOH (50 mL) was added K₂CO₃ (3.61 g, 26.1 mmol). The mixture was stirred at 20° C. for 2 hours. On completion, the reaction mixture was concentrated in vacuo to give the title compound (2.2 g) as a brown solid. LC-MS (ESI⁺) m/z 284.1 (M+Na)⁺.

Step 4-(4-ethynyl-2-methoxyphenyl)methanamine. To a solution of tert-butyl N-[(4-ethynyl-2-methoxy-phenyl)methyl]carbamate (2.2 g, 8.42 mmol) in DCM (50 mL) was added HCl/dioxane (4 M, 40 mL).The mixture was stirred at 25° C. for 1 hour. On completion, the mixture was concentrated under reduced pressure to give the title compound (1.3 g) as brown solid. LC-MS (ESI⁺) m/z 162.2 (M+H)⁺.

### (2S,4R)-1-((S)-2-Amino-3,3-dimethylbutanoyl)-N-(4-ethynyl-2-methoxybenzyl)-4-hydroxypyrrolidine-2-carboxamide (Intermediate HP)

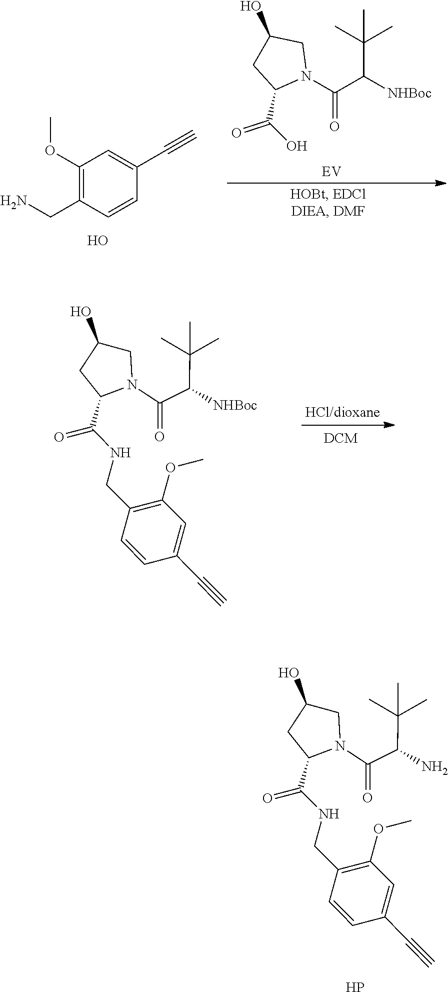

Step 1-Tert-butyl ((S)-1-((2S,4R)-2-((4-ethynyl-2-methoxybenzyl)carbamoyl)-4-hydroxypyrrolidin-1-yl)-3,3-dimethyl-1-oxobutan-2-yl)carbamate. To a solution of (4-ethynyl-2-methoxy-phenyl)methanamine (714 mg, 2.53 mmol, HCl, Intermediate HO) in DMF (10 mL) was added EDCI (727 mg, 3.80 mmol) and HOBt (513 mg, 3.80 mmol). The mixture was stirred at 0° C. for 0.5 hour, then added (2S,4R)-1-[(2S)-2-(tert-butoxycarbonylamino)-3,3-dimethyl-butanoyl]-4-hydroxy-pyrrolidine-2-carboxylic acid (871 mg, 2.53 mmol, Intermediate EV) and DIEA (1.63 g, 12.7 mmol, 2.20 mL) was added and the mixture was stirred at 25° C. for 1.5 hours. On completion, the reaction mixture was partitioned between H₂O (30 mL) and ethyl acetate (50 mL). The organic phase was separated, washed with brine (20 mL×3), dried over dry Na₂SO₄, filtered and concentrated under reduced pressure to give a residue. The residue was purified by column chromatography (SiO₂, Petroleum ether/Ethyl acetate=2/1 to 0/1) to give the title compound (9.20 mg, 14% yield) as a white solid. LC-MS (ESI⁺) m/z 488.2 (M+H)⁺.

Step 2-(2S,4R)-1-((S)-2-amino-3,3-dimethylbutanoyl)-N-(4-ethynyl-2-methoxybenzyl)-4-hydroxypyrrolidine-2-carboxamide. To a solution of tert-butyl N-[(1S)-1-[(2S,4R)-2-[(4-ethynyl-2-methoxy-phenyl)methylcarbamoyl]-4-hydroxy-pyrrolidine-1-carbonyl]-2,2-dimethyl-propyl]carbamate (250 mg, 513 umol) in DCM (10 mL) was added HCl/dioxane (4 M, 2.50 mL). The mixture was stirred at 25° C. for 1 hour. On completion, the mixture was concentrated under reduced pressure to give the title compound (264 mg) as gray solid. LC-MS (ESI⁺) m/z 388.2 (M+H)⁺.

### (2S,4R)—N-(4-ethynylbenzyl)-4-hydroxypyrrolidine-2-carboxamide (Intermediate HQ)

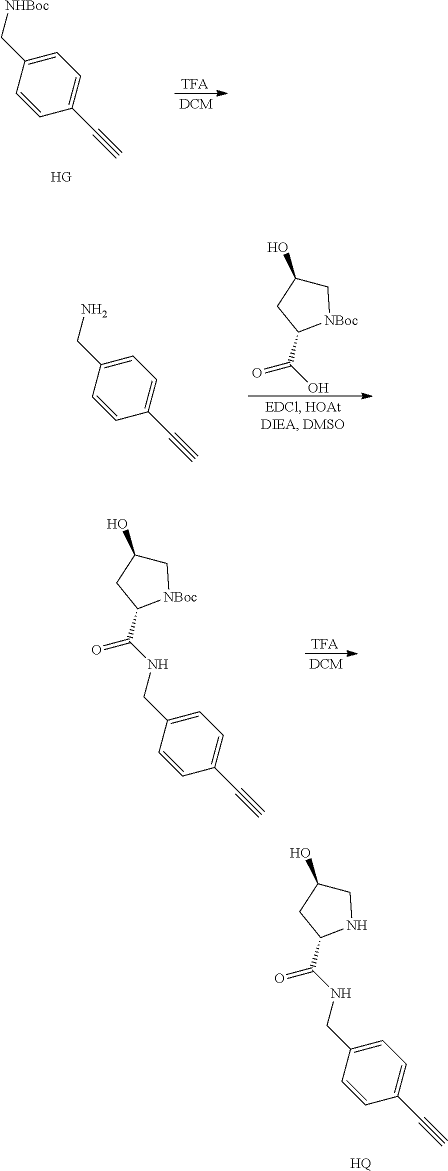

Step 1-(2S,4R)-tert-butyl 2-((4-ethynylbenzyl)carbamoyl)-4-hydroxypyrrolidine-1-carboxylate. To a solution of (4-ethynylphenyl)methanamine (1.5 g, 6.12 mmol, TFA, Intermediate HG) in DMSO (15 mL) was added EDCI (1.17 g, 6.12 mmol) and HOAt (833 mg, 6.12 mmol, 856 uL) DIEA (3.95 g, 30.6 mmol, 5.33 mL) and (2S,4R)-1-tert-butoxycarbonyl-4-hydroxy-pyrrolidine-2-carboxylic acid (1.41 g, 6.12 mmol, CAS #13726-69-7). The mixture was stirred at 25° C. for 12 hours. The reaction mixture was diluted with EA 20 mL and extracted with EA 90 mL (30 mL×3). The combined organic layers were washed with sat. NaCl 150 mL (50 mL×3), dried over Na₂SO₄, filtered and concentrated under reduced pressure to give a residue. The crude product was purified by MPLC(SiO₂, PE:EA=1:1 to 0:1) to give the title compound (1.8 g, 74% yield) as a white solid. LC-MS (ESI⁺) m/z 245.3 (M−100+H)⁺.

Step 2-(2S,4R)—N-(4-ethynylbenzyl)-4-hydroxypyrrolidine-2-carboxamide. To a solution of tert-butyl (2S,4R)-2-[(4-ethynylphenyl)methylcarbamoyl]-4-hydroxy-pyrrolidine-1-carboxylate (0.5 g, 2 mmol) in DCM (5 mL) was added TFA (1.54 g, 13.5 mmol, 1 mL). The mixture was stirred at 25° C. for 30 mins. The mixture was concentrated under reduce pressure to give the title compound (100 mg, TFA) as a white solid. LC-MS (ESI⁺) m/z 245.3 (M+H)⁺.

### Tert-butyl 4-(2-chloropyrimidin-5-yl)-5,6-dihydropyridine-1(2H)-carboxylate (Intermediate CX)

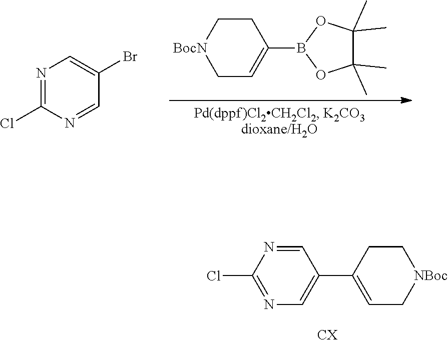 [SMILES: Clc1ncc(Br)cn1 ; CC(C)(C)OC(=O)N1CC=C(B2OC(C)(C)C(C)(C)O2)CC1 ; "Pd(dppf)Cl2·CH2Cl2, K2CO3dioxane/H2O" ; CC(C)(C)OC(=O)N1CC=C(c2cnc(Cl)nc2)CC1]

A mixture of 5-bromo-2-chloro-pyrimidine (60 g, 310 mmol, CAS #32779-36-5), tert-butyl 4-(4,4,5,5-tetramethyl-1,3,2-dioxaborolan-2-yl)-5,6-dihydropyridine-1(2H)-carboxylate (106 g, 341 mmol, CAS #286961-14-6), K₂CO₃ (129 g, 931 mmol), Pd(dppf)Cl₂·CH₂Cl₂ (12.7 g, 15.5 mmol) in dioxane (400 mL) and H₂O (100 mL) was degassed and purged with N₂ three times. Then the mixture was stirred at 100° C. for 12 hours under N₂ atmosphere. On completion, the reaction mixture was extracted with EA (3×100 mL). The combined organic layers were washed with brine, dried over Na₂SO₄, filtered and concentrated under reduced pressure to give a residue. The residue was purified by column chromatography (SiO₂, Petroleum ether/Ethyl acetate=3/1) to give the title compound (80.3 g, 51% yield) as a pink oil. LC-MS (ESI⁺) m/z 296.2 (M+H)⁺.

### (S)-2-(6-(5-(1-(2-azaspiro[3.3]heptan-6-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate HT)

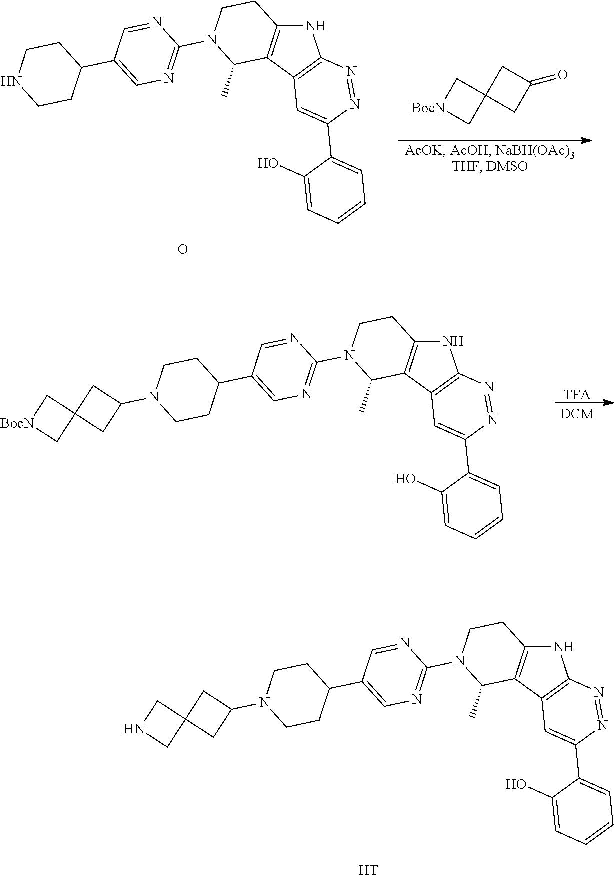

Step 1-(S)-tert-butyl 6-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate. To a solution of 2-[(3S)-3-methyl-4-[5-(4-piperidyl)pyrimidin-2-yl]-4,8,10,11-tetrazatricyclo[7.4.0.02,7]trideca-1(9),2(7),10,12-tetraen-12-yl]phenol (20 g, 41.8 mmol, HCl, Intermediate O) in DMSO (200 mL) and THF (80 mL) was added KOAc (12.3 g, 125 mmol) at 25° C. and the mixture was stirred for 0.5 hr. Then tert-butyl 6-oxo-2-azaspiro[3.3]heptane-2-carboxylate (10.6 g, 50.2 mmol, 1.2) and HOAc (7.54 g, 125 mmol, 7.18 mL) was added to stirred at 25° C. and the mixture was stirred for 0.5 hr. Finally, NaBH(OAc)₃ (22.1 g, 104 mmol) was added to stirred at 0-25° C. for 3 hrs. On completion, the residue was diluted with Na₂CO₃ solution (1000 mL) and filtered and concentrated under reduced pressure to give a residue. The residue was purified by column chromatography (SiO2, DCM: MeOH=30:1) to give (20 g, 63% yield, FA) as a yellow solid. LC-MS (ESI⁺) m/z=637.5 (M+H)⁺.

Step 2-(S)-2-(6-(5-(1-(2-azaspiro[3.3]heptan-6-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 6-[4-[2-[(3R)-12-(2-hydroxyphenyl)-3-methyl-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carboxylate (12 g, 18.8 mmol) in DCM (150 mL) was added TFA (30 mL), then the mixture was stirred at 25° C. for 0.5 hour. On completion, the reaction mixture was concentrated under reduced pressure to remove DCM. The residue was purified by prep-HPLC (0.1% FA) to give (10 g, 91% yield, FA) as a yellow solid. LC-MS (ESI⁺) m/z=537.5 (M+H)⁺.

### Phenyl-((S)-1-((2S,4R)-2-((4-ethynyl-2-methoxybenzyl)carbamoyl)-4-hydroxypyrrolidin-1-yl)-3,3-dimethyl-1-oxobutan-2-yl)carbamate (Intermediate HU)

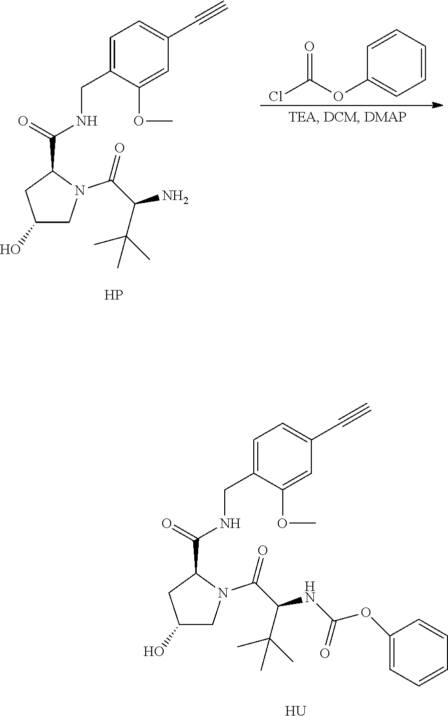

To a solution of phenyl carbonochloridate (26.26 mg, 167.8 umol) in DCM (1 mL) was added TEA (52.23 mg, 516.17 umol) and DMAP (3.15 mg, 25.81 umol) at 0° C. for 0.5 h. Then, (2S,4R)-1-((S)-2-amino-3,3-dimethylbutanoyl)-N-(4-ethynyl-2-methoxybenzyl)-4-hydroxypyrrolidine-2-carboxamide (50 mg, 129.04 umol, Intermediate HP) was added and the mixture was stirred at 0° C. for 0.5 hr. On completion, the reaction mixture was concentrated in vacuo to give a residue. The residue was purified by column chromatography (SiO₂, Petroleum ether/Ethyl acetate=1/2) to give the title compound (18 mg, 27% yield) as a white solid. LC-MS (ESI⁺) m/z 508.2 (M+H)⁺.

### Phenyl ((S)-1-((2S,4R)-2-((4-ethynyl-2-fluorobenzyl)carbamoyl)-4-hydroxypyrrolidin-1-yl)-3,3-dimethyl-1-oxobutan-2-yl)carbamate (Intermediate HV)

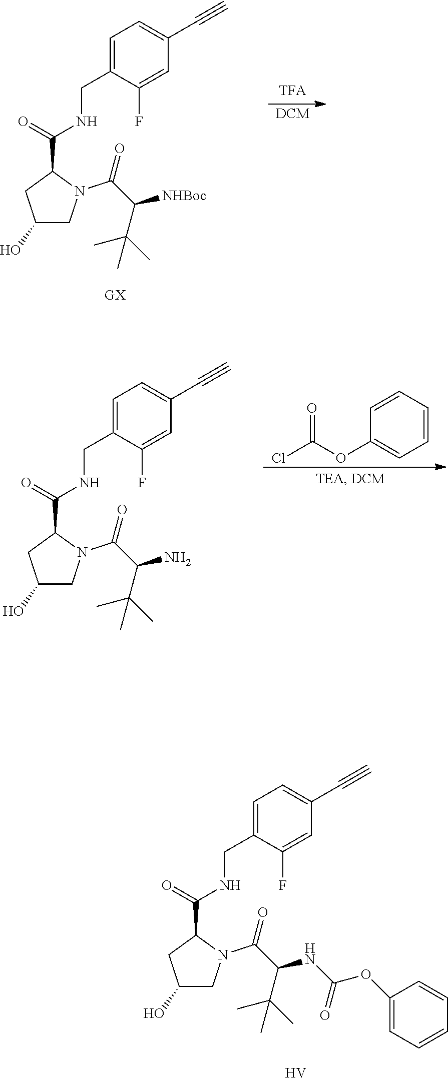

Step 1-(2S,4R)-1-((S)-2-amino-3,3-dimethylbutanoyl)-N-(4-ethynyl-2-fluorobenzyl)-4-hydroxypyrrolidine-2-carboxamide. To a solution of tert-butyl ((S)-1-((2S,4R)-2-((4-ethynyl-2-fluorobenzyl)carbamoyl)-4-hydroxypyrrolidin-1-yl)-3,3-dimethyl-1-oxobutan-2-yl)carbamate (600.00 mg, 1.26 mmol, Intermediate GX) in DCM (5 mL) was added TFA(1.54 g, 13.51 mmol). The mixture was stirred at 25° C. for 0.5 hour. On completion, the reaction mixture was concentrated in vacuo to give a residue. The residue was purified by prep-HPLC (neutral condition) to give the title compound (300 mg, 62% yield) as a white solid. LC-MS (ESI⁺) m/z 376.2 (M+H)⁺.

Step 2-Phenyl ((S)-1-((2S,4R)-2-((4-ethynyl-2-fluorobenzyl)carbamoyl)-4-hydroxypyrrolidin-1-yl)-3,3-dimethyl-1-oxobutan-2-yl)carbamate. To a mixture of (2S,4R)-1-((S)-2-amino-3,3-dimethylbutanoyl)-N-(4-ethynyl-2-fluorobenzyl)-4-hydroxypyrrolidine-2-carboxamide (250 mg, 666 umol) in DCM (2 mL) was added DIEA (344 mg, 2.66 mmol) and phenyl carbonochloridate (125.11 mg, 799.07 umol). The mixture was stirred at 25° C. for 0.5 hour. On completion, the reaction mixture was concentrated in vacuo to give a residue. The residue was purified by column chromatography (SiO₂, Petroleum ether/Ethyl acetate=1/1) to give the title compound (340 mg, 99% yield) as a white solid. LC-MS (ESI⁺) m/z 496.1 (M+H)⁺.

### (S)-2-(8-(5-Bromopyridin-2-yl)-6,6a,7,8,9,10-hexahydro-5H-pyrazino[1′,2′:4,5]pyrazino[2,3-c]pyridazin-2-yl)phenol (Intermediate HW)

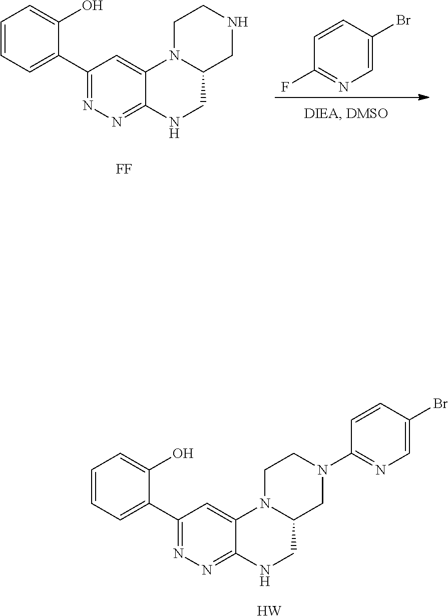

To a solution of 2-[(1OR)-1,5,6,8,12-pentazatricyclo[8.4.0.0^{2,7}]tetradeca-2,4,6-trien-4-yl]phenol (1.5 g, 4.69 mmol, HCl, Intermediate FF) and 5-bromo-2-fluoro-pyridine (990 mg, 5.63 mmol, CAS #766-11-0) in DMSO (20 mL) was added DIEA (1.82 g, 14.0 mmol). The mixture was stirred at 120° C. for 12 hours. On completion, the reaction mixture was quenched with H₂O (20 mL) and extracted with EA (100 mL×3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=3/1 to 1/3) to give the title compound (640 mg, 29% yield) as a brown solid. LC-MS (ESI⁺) m/z 439.0 (M+H)⁺.

### (S)-2-(8-(5-(piperidin-4-yl)pyridin-2-yl)-6,6a,7,8,9,10-hexahydro-5H-pyrazino[1′,2′:4,5]pyrazino[2,3-c]pyridazin-2-yl)phenol (Intermediate HX)

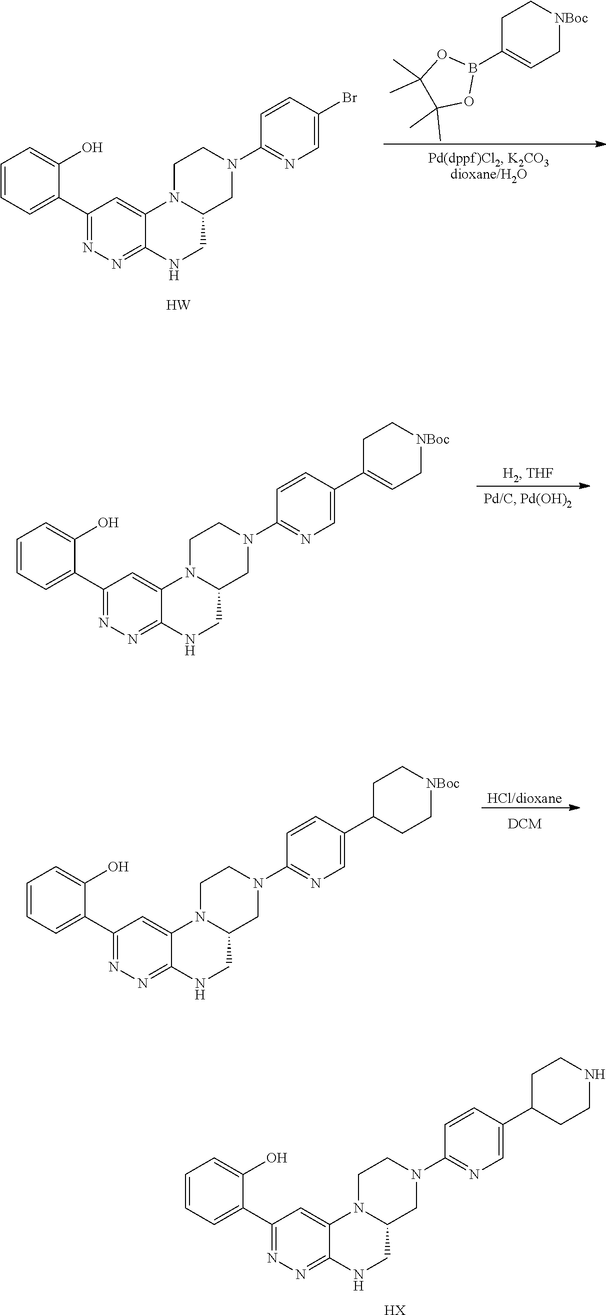

Step 1-(S)-tert-butyl 6-(2-(2-hydroxyphenyl)-6a,7,9,10-tetrahydro-5H-pyrazino[1′,2′:4,5]pyrazino[2,3-c]pyridazin-8(6H)-yl)-5′,6′-dihydro-[3,4′-bipyridine]-1′(2′H)-carboxylate. To a solution of 2-[(10S)-12-(5-bromo-2-pyridyl)-1,5,6,8,12-pentazatricyclo[8.4.0.0^{2,7}]tetradeca-2,4,6-trien-4-yl]phenol (500 mg, 1 mmol, Intermediate HW), tert-butyl 4-(4,4,5,5-tetramethyl-1,3,2-dioxaborolan-2-yl)-3,6-dihydro-2H-pyridine-1-carboxylate (527 mg, 1.71 mmol, CAS #286961-14-6) in dioxane (5 mL) and H₂O (1 mL) was added K₂CO₃ (471 mg, 3.41 mmol) and Pd(dppf)Cl₂ (83.3 mg, 113 umol). The mixture was degassed and purged with N₂ three times. Then the mixture was stirred at 80° C. for 12 hours under N₂ atmosphere. On completion, the reaction mixture was diluted with H₂O (10 mL) and extracted with EA (50 mL×3). The combined organic layers were washed with brine (30 mL×2), dried over Na₂SO₄, filtered and concentrated under reduced pressure to give a residue. The residue was purified by column chromatography (SiO₂, Petroleum ether/Ethyl acetate=1/1 to SiO₂, DCM: MeOH=10:1) to give the title compound (420 mg, 51% yield) as a brown solid. LC-MS (ESI⁺) m/z 542.6 (M+H)⁺.

Step 2-(S)-tert-butyl 4-(6-(2-(2-hydroxyphenyl)-6a,7,9,10-tetrahydro-5H-pyrazino[1′,2′:4,5]pyrazino[2,3-c]pyridazin-8(6H)-yl)pyridin-3-yl)piperidine-1-carboxylate. To a solution of tert-butyl 4-[6-[(10S)-4-(2-hydroxyphenyl)-1,5,6,8,12-pentazatricyclo[8.4.0.0^{2,7}]tetradeca-2,4,6-trien-12-yl]-3-pyridyl]-3,6-dihydro-2H-pyridine-1-carboxylate (300 mg, 553 umol) in THF (30 mL) was added Pd(OH)₂ (150 mg, 106 umol, 10 wt %) and Pd/C (150 mg, 10 wt %). The mixture was stirred at 25° C. for 12 hours under H₂ atmosphere. On completion, the reaction mixture was filtered and concentrated under reduced pressure to give the title compound (270 mg) as a yellow solid. LC-MS (ESI⁺) m z 544.5 (M+H)⁺.

Step 3-(S)-2-(8-(5-(piperidin-4-yl)pyridin-2-yl)-6,6a,7,8,9,10-hexahydro-5H-pyrazino[1′,2′:4,5]pyrazino[2,3-c]pyridazin-2-yl)phenol. To a solution of tert-butyl 4-[6-[(10S)-4-(2-hydroxyphenyl)-1,5,6,8,12-pentazatricyclo[8.4.0.0^{2,7}]tetradeca-2,4,6-trien-12-yl]-3-pyridyl]piperidine-1-carboxylate (270 mg, 496 umol) in DCM (10 mL) was added HCl/dioxane (5 M, 1 mL). The mixture was stirred at 25° C. for 1 hour. On completion, the reaction mixture was filtered and concentrated under reduced pressure to give a residue. The residue was purified by prep-HPLC (FA condition) to give the title compound (250 mg, 94% yield, FA) as a yellow solid. LC-MS (ESI⁺) m/z 444.1 (M+H)⁺.

### (4-Ethynyl-2-(3-methoxypropoxy)phenyl)methanamine (Intermediate HY)

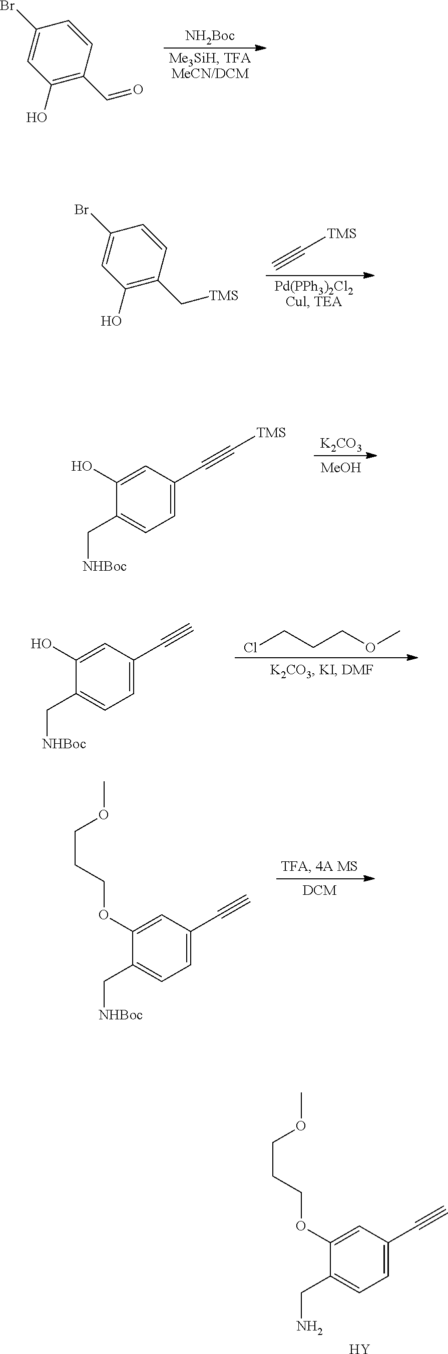

Step 1-Tert-butyl 4-bromo-2-hydroxybenzylcarbamate. To a solution of 4-bromo-2-hydroxy-benzaldehyde (30 g, 150 mmol) in DCM (100 mL) ACN (300 mL) was added Et₃SiH (52.06 g, 447.72 mmol, 71.51 mL) and TFA (34.03 g, 298.5 mmol, 22.10 mL) and tert-butyl carbamate (52.45 g, 447.7 mmol). The mixture was stirred at 25° C. for 12 hr. On completion, the reaction mixture was concentrated under reduced pressure to give a residue. The crude product was purified by MPLC (SiO₂,PE:EA=50:1 to 10:1) to give the title compound (58 g, 97% yield) as a white solid. LC-MS (ESI⁺) m/z 326.1 (M+Na)⁺.

Step 2-Tert-butyl 2-hydroxy-4-((trimethylsilyl)ethynyl)benzylcarbamate. A mixture of tert-butyl N-[(4-bromo-2-hydroxy-phenyl)methyl]carbamate (35 g, 120 mmol), ethynyl(trimethyl)silane (91.0 g, 927 mmol, 128 mL), Pd(PPh₃)₂Cl₂ (4.07 g, 5.79 mmol), CuI (2.21 g, 11.6 mmol) in TEA (300 mL) was degassed and purged with N₂ three times. Then the mixture was stirred at 80° C. for 12 hours. The reaction mixture was quenched with sat. NH₄Cl (100 mL) at 25° C., and then diluted with ethyl acetate (100 mL) and extracted with ethyl acetate (150 mL×3). The combined organic layers were washed with sat. NaCl (300 mL×1), dried over Na₂SO₄, filtered and concentrated under reduced pressure to give a residue. The crude product was purified by MPLC (SiO₂,PE:EA=10/1 to 1/1) to give the title compound (12 g, 31% yield) as white solid; LC-MS (ESI⁺) m/z 342.2 (M+Na)⁺.

Step 3-2-(Aminomethyl)-5-ethynylphenol. To a solution of tert-butyl N-[[2-hydroxy-4-(2-trimethylsilylethynyl)phenyl]methyl]carbamate (0.6 g, 2 mmol) in MeOH (7 mL) was added K₂CO₃ (519.13 mg, 3.76 mmol). The mixture was then stirred at 20° C. for 12 hr. On completion, the mixture was concentrated under reduce pressure to give the title compound (480 mg) as white solid. ¹H NMR (400 MHz, DMSO-d₆) δ 9.76 (s, 1H), 7.24-7.15 (m, 1H), 7.08-6.99 (m, 1H), 6.98-6.91 (m, 1H), 6.90-6.83 (m, 1H), 4.11-3.99 (m, 3H), 1.39 (s, 9H).

Step 4-Tert-butyl 4-ethynyl-2-(3-methoxypropoxy)benzylcarbamate. To a solution of tert-butyl N-[(4-ethynyl-2-hydroxy-phenyl)methyl]carbamate (1.5 g, 6.1 mmol) in DMF (30 mL) was added K₂CO₃ (2.51 g, 18.2 mmol) and KI (201 mg, 1.21 mmol) and 1-chloro-3-methoxy-propane (1.65 g, 15.2 mmol). The mixture was stirred at 80° C. for 12 hours. On completion, the reaction mixture was quenched with sat. NH₄Cl (5 m)L at 25° C., and then diluted with EA (10 mL) and extracted with EA (10 mL×3). The combined organic layers were washed with sat. NaCl (10 mL×3), dried over Na₂SO₄, filtered and concentrated under reduced pressure to give a residue. The crude product was purified by MPLC (SiO₂, PE:EA=5:1 to 1:1) to give the title compound (1.3 g, 55% yield) as a white solid. ¹H NMR (400 MHz, DMSO-d₆) δ 7.02-6.94 (m, 1H), 6.90 (d, J=7.6 Hz, 1H), 6.87-6.70 (m, 2H), 3.93-3.86 (m, 2H), 3.83 (t, J=6.0 Hz, 2H), 3.28 (t, J=6.4 Hz, 2H), 3.11 (s, 1H), 3.04 (s, 3H), 1.74 (q, J=6.4 Hz, 2H), 1.23-1.07 (m, 9H); LC-MS (ESI⁺) m/z 342.2 (M+Na)⁺.

Step 5-(4-Ethynyl-2-(3-methoxypropoxy)phenyl)methanamine. To a solution of tert-butyl N-[[4-ethynyl-2-(3-methoxypropoxy)phenyl]methyl]carbamate (1.3 g, 4.07 mmol) in DCM (3 mL) was added TFA (2.61 g, 22.9 mmol, 1.70 mL) and 4 Å molecular sieves (0.3 g). The mixture was stirred at 25° C. for 30 mins. On completion, the mixture was concentrated under reduce pressure to give the title compound (2 g, TFA) as a white solid. LC-MS (ESI⁺) m/z 220.2 (M+H)⁺.

### Phenyl ((S)-1-((2S,4R)-2-((4-ethynyl-2-(3-methoxypropoxy)benzyl)carbamoyl)-4-hydroxypyrrolidin-1-yl)-3,3-dimethyl-1-oxobutan-2-yl)carbamate (Intermediate HZ)

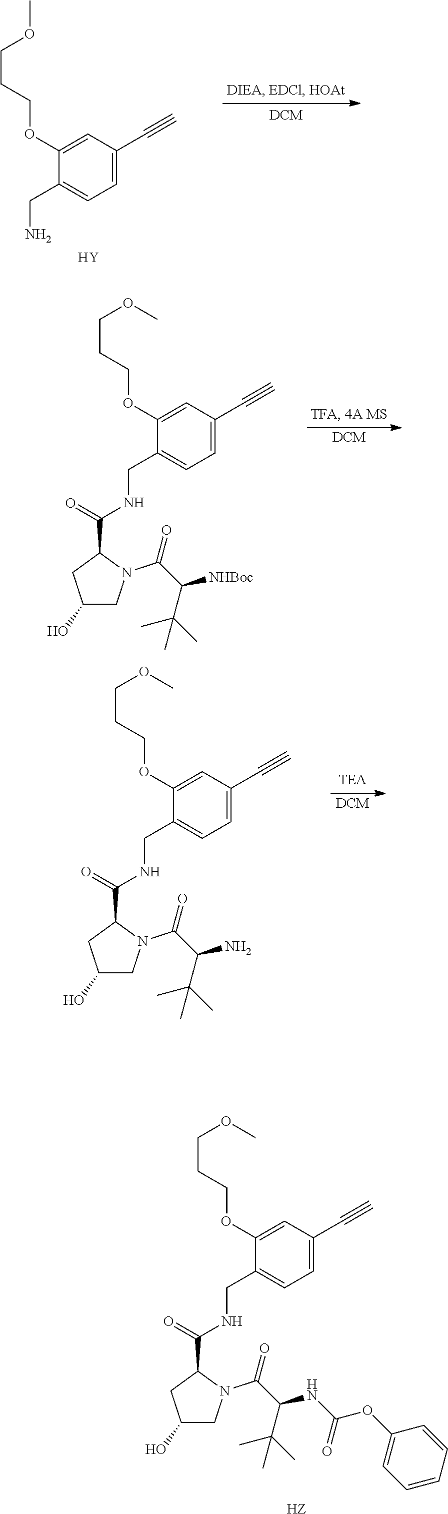

Step 1-Tert-butyl ((S)-1-((2S,4R)-2-((4-ethynyl-2-(3-methoxypropoxy)benzyl)carbamoyl)-4-hydroxypyrrolidin-1-yl)-3,3-dimethyl-1-oxobutan-2-yl)carbamate. To a solution of (2S,4R)-1-[2-(tert-butoxycarbonylamino)-3,3-dimethyl-butanoyl]-4-hydroxy-pyrrolidine-2-carboxylic acid (2.07 g, 6.00 mmol, Intermediate EV) and [4-ethynyl-2-(3-methoxypropoxy)phenyl]methanamine (2 g, 6.00 mmol, TFA, Intermediate HY) in DCM (35 mL) was added DIEA (3.88 g, 30.0 mmol, 5.23 mL), EDCI (1.38 g, 7.20 mmol) and HOAt (980 mg, 7.20 mmol, 1.01 mL). The mixture was stirred at 25° C. for 12 hours. On completion, the mixture was concentrated under reduce pressure to give a residue. The crude reaction mixture was purified by MPLC (SiO₂, PE:EA=1:1 to 1:2) to give the title compound (1.7 g, 42% yield) as a white solid. ¹H NMR (400 MHz, DMSO-d₆) δ 8.54-8.32 (m, 1H), 7.34 (d, J=7.6 Hz, 1H), 7.00 (s, 1H), 6.93 (d, J=8.0 Hz, 1H), 6.46 (d, J=9.2 Hz, 1H), 5.14 (d, J=2.8 Hz, 1H), 4.46 (t, J=8.0 Hz, 1H), 4.35 (s, 1H), 4.30-4.20 (m, 1H), 4.18-4.09 (m, 2H), 4.05 (t, J=6.0 Hz, 2H), 3.68-3.56 (m, 2H), 3.49 (t, J=6.4 Hz, 2H), 3.25 (s, 3H), 2.69 (s, 1H), 1.96 (d, J=12.4 Hz, 4H), 1.39 (s, 9H), 0.99-0.81 (m, 9H); LC-MS (ESI⁺) m/z 546.5 (M+H)⁺.

Step 2-(2S,4R)-1-((S)-2-amino-3,3-dimethylbutanoyl)-N-(4-ethynyl-2-(3-methoxypropoxy)benzyl)-4-hydroxypyrrolidine-2-carboxamide (Intermediate JP). To a solution of tert-butyl N-[1-[(2S,4R)-2-[[4-ethynyl-2-(3-methoxypropoxy)phenyl]methylcarbamoyl]-4-hydroxy-pyrrolidine-1-carbonyl]-2,2-dimethyl-propyl]carbamate (1.4 g, 2.6 mmol) in DCM (15 mL) was added TFA (4.62 g, 40.5 mmol, 3 mL) and 4 Å molecular sieves (2.57 mmol). The mixture was stirred at 25° C. for 30 mins. On completion, the mixture was concentrated under reduce pressure to give a residue. The crude product was purification by reversed-phase flash chromatography(0.1% NH₃—H₂O condition) to give the title compound (750 mg, 60% yield) as a white solid. LC-MS (ESI⁺) m/z 446.3 (M+H)⁺.

Step 3-phenyl ((S)-1-((2S,4R)-2-((4-ethynyl-2-(3-methoxypropoxy)benzyl)carbamoyl)-4-hydroxypyrrolidin-1-yl)-3,3-dimethyl-1-oxobutan-2-yl)carbamate. To a solution of (2S,4R)-1-[(2S)-2-amino-3,3-dimethyl-butanoyl]-N-[[4-ethynyl-2-(3-methoxypropoxy)phenyl]methyl]-4-hydroxy-pyrrolidine-2-carboxamide (0.3 g, 700 umol) in DCM (5 mL) was added TEA (204 mg, 2.02 mmol, 281 uL) and phenyl carbonochloridate (127 mg, 808 umol, 101 uL) at 0° C. The mixture was stirred at 0° C. for 30 mins. On completion, the reaction mixture was quenched with sat. NH₄Cl 5 mL at 25° C., and then diluted with DCM (5 mL) and extracted with DCM (10 mL×3). The combined organic layers were washed with sat. NaCl (30 mL×1), dried over Na₂SO₄, filtered and concentrated under reduced pressure to give a residue. The crude reaction mixture was purified by MPLC (SiO₂, PE:EA=1:1 to 1:2). to give the title compound (170 mg, 41% yield) as a white solid. LC-MS (ESI⁺) m/z 566.4 (M+H)⁺.

### 2-Fluoro-6-(6,6a,7,8,9,10-hexahydro-5H-pyrazino[1′,2′:4,5]pyrazino[2,3-c]pyridazin-2-yl)phenol (Intermediate IA)

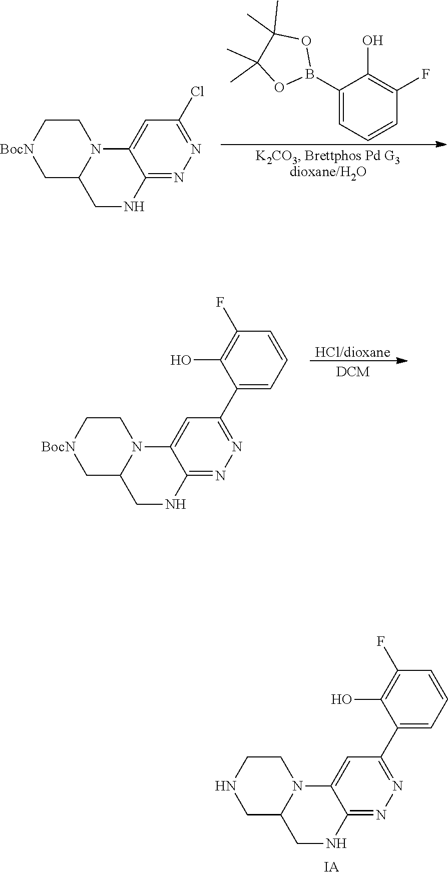

Step 1-Tert-butyl 2-(3-fluoro-2-hydroxyphenyl)-6a,7,9,10-tetrahydro-5H-pyrazino[1′,2′:4,5]pyrazino[2,3-c]pyridazine-8(6H)-carboxylate. To a solution of tert-butyl 4-chloro-1,5,6,8,12-pentazatricyclo[8.4.0.0^{2,7}]tetradeca-2,4,6-triene-12-carboxylate (3 g, 9 mmol, synthesized via Steps 1-2 of Intermediate FD) in dioxane (15 mL), H₂O (3 mL) was added K₂CO₃ (3.82 g, 27.6 mmol), Brettphos (1.79 g, 1.84 mmol), and (3-fluoro-2-hydroxy-phenyl) boronic acid (2.87 g, 18.4 mmol CAS #259209-24-0). The mixture was stirred at 110° C. for 12 hours under N₂ atmosphere. On completion, the reaction mixture was diluted with water (20 mL) and extracted with DCM (20 mL×3). The combined organic layers were washed with aqueous NaCl (20 mL×3), dried over Na₂SO₄, filtered and concentrated under reduced pressure to give a residue. The residue was purified by prep-HPLC (FA condition) to give the title compound (1.5 g, 31% yield) as a yellow solid. LC-MS (ESI⁺) m/z 402.2 (M+H)⁺.

Step 2-2-Fluoro-6-(6,6a,7,8,9,10-hexahydro-5H-pyrazino[1′,2′:4,5]pyrazino[2,3-c]pyridazin-2-yl)phenol. To a solution of tert-butyl 4-(3-fluoro-2-hydroxy-phenyl)-1,5,6,8,12-pentazatricyclo[8.4.0.0^{2,7}]tetradeca-2,4,6-triene-12-carboxylate (1.5 g, 3.7 mmol) in DCM (10 mL) was added HCl/dioxane (5 M, 12.00 mL). The mixture was stirred at 25° C. for 2 hours. On completion, the reaction mixture was concentrated under reduced pressure to give the residue (1.6 g) as a yellow solid. LC-MS (ESI⁺) m/z 302.0 (M+H)⁺.

### (R)-2-Fluoro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate IB) and (S)-2-fluoro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate IC)

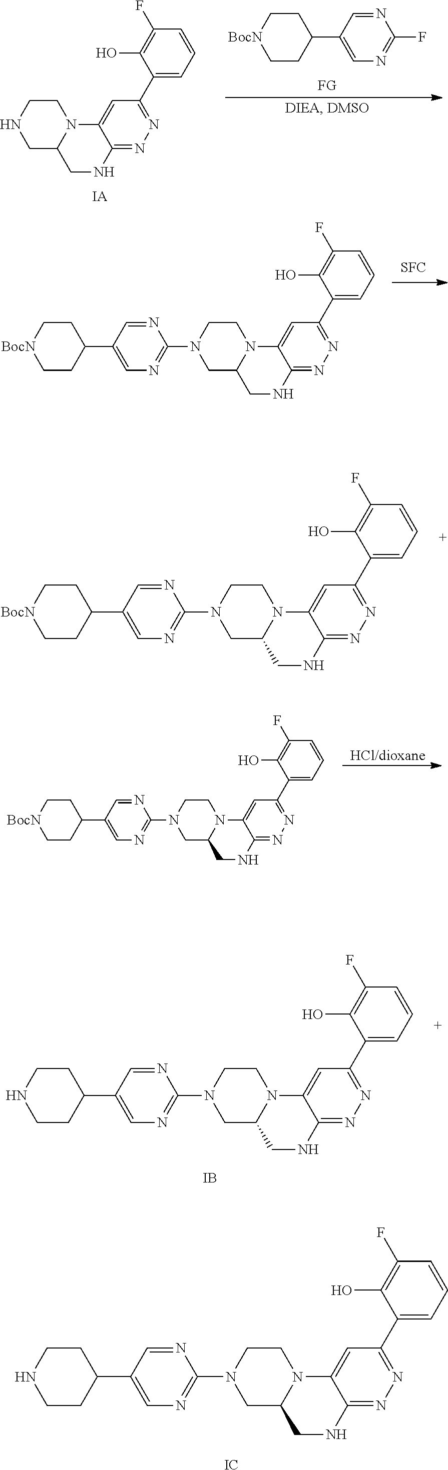

Step 1-Tert-butyl 4-(2-(2-(3-fluoro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate. To a solution of 2-fluoro-6-(1,5,6,8,12-pentazatricyclo[8.4.0.0^{2,7}]tetradeca-2,4,6-trien-4-yl)phenol (1.6 g, 5.3 mmol, Intermediate IA) in DMSO (15 mL) was added DIEA (3.43 g, 26.5 mmol, 4.62 mL) and tert-butyl 4-(2-fluoropyrimidin-5-yl)piperidine-1-carboxylate (2.02 g, 7.17 mmol, Intermediate FG). The mixture was stirred at 80° C. for 3 hrs. On completion, the reaction mixture was diluted with water (20 mL) and extracted with DCM (20 mL×3). The combined organic layers were washed with aqueous NaCl (20 mL×3), dried over Na₂SO₄, filtered and concentrated under reduced pressure to give a residue. The residue was purified by column chromatography (SiO₂, DCM/MeOH=60/1 to 20/1) to give the title compound (2 g, 66% yield) as a white solid. LC-MS (ESI⁺) m/z 563.4 (M+H)⁺.

Step 2-Tert-butyl (R)-4-(2-(2-(3-fluoro-2-hydroxyphenyl)-5,6,6a,7,9,10-hexahydro-8H-pyrazino[1′,2′:4,5]pyrazino[2,3-c]pyridazin-8-yl)pyrimidin-5-yl)piperidine-1-carboxylate and tert-butyl (S)-4-(2-(2-(3-fluoro-2-hydroxyphenyl)-5,6,6a,7,9,10-hexahydro-8H-pyrazino[1′,2′:4,5]pyrazino[2,3-c]pyridazin-8-yl)pyrimidin-5-yl)piperidine-1-carboxylate. Tert-butyl 4-(2-(2-(3-fluoro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate was purified by SFC (column: DAICEL CHIRALPAK AD(250 mm*30 mm, 10 um); mobile phase: [0.1% NH₃H2O IPA]; B %: 45%-45%, 3.31; 200 min) to give tert-butyl (R)-4-(2-(2-(3-fluoro-2-hydroxyphenyl)-5,6,6a,7,9,10-hexahydro-8H-pyrazino[1′,2′:4,5]pyrazino[2,3-c]pyridazin-8-yl)pyrimidin-5-yl)piperidine-1-carboxylate (rt: 1.128 min, 0.56 g, 16% yield) (LC-MS (ESI⁺) m/z 563.3 (M+H)⁺) and tert-butyl (S)-4-(2-(2-(3-fluoro-2-hydroxyphenyl)-5,6,6a,7,9,10-hexahydro-8H-pyrazino[1′,2′:4,5]pyrazino[2,3-c]pyridazin-8-yl)pyrimidin-5-yl)piperidine-1-carboxylate (rt: 1.854 min, 2.0 g, 65% yield) (LC-MS (ESI⁺) m/z 563.4 (M+H)⁺). Absolute stereochemistry was assigned arbitrarily.

Step 3-(R)-2-fluoro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol and (S)-2-fluoro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol. To solutions of tert-butyl (R)-4-(2-(2-(3-fluoro-2-hydroxyphenyl)-5,6,6a,7,9,10-hexahydro-8H-pyrazino[1′,2′:4,5]pyrazino[2,3-c]pyridazin-8-yl)pyrimidin-5-yl)piperidine-1-carboxylate (50 mg, 88.8 umol) and (S)-2-fluoro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (50 mg, 88.8 umol) was added HCl/dioxane (4 M, 444.3 uL). The mixtures were stirred at 25° C. for 2 hours. On completion, the reaction mixture was concentrated under reduced pressure to give the residue. The products were purified by pre-HPLC (column: Phenomenex luna C18 150*25 mm*10 um; mobile phase: [water (FA) -ACN]; B %: 1% −28%, 10.5 min) to give (R)-2-fluoro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (17.4 mg, 38% yield) as a white solid (LC-MS (ESI⁺) m/z 463.2 (M+H)⁺; ¹H NMR (400 MHz, DMSO-d₆) δ ppm 8.39 (s, 1H), 8.34 (s, 2H), 7.79 (d, J=8.25 Hz, 1H), 7.53 (d, J=2.80 Hz, 1H), 7.34 (s, 1H), 7.12-7.21 (m, 1H), 6.77-6.88 (m, 1H), 4.72 (t, J=8.80 Hz, 2H), 4.21 d, J=12.0 Hz, 2H), 3.60-3.66 (m, 1H), 3.34-3.41 (m, 1H), 3.19-3.30 (m, 3H), 3.10-3.17 (m, 1H), 2.99 (td, J=12.0, 3.20 Hz, 1H), 2.74-2.85 (m, 3H), 2.62-2.70 (m, 1H), 1.78-1.88 (m, 2H), 1.64-1.78 (m, 2H)) and (S)-2-fluoro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (13.3 mg, 32% yield) as a white solid (LC-MS (ESI⁺) m/z 463.2 (M+H)⁺, ¹H NMR (400 MHz, DMSO-d₆) δ ppm 8.31-8.41 (m, 3H), 7.79 (d, J=7.60 Hz, 1H), 7.53 (s, 1H), 7.34 (s, 1H), 7.17 (t, J=9.60 Hz, 1H), 6.74-6.89 (m, 1H), 4.73 (d, J=8.20 Hz, 2H), 4.21 (d, J=12.0 Hz, 2H), 3.63 (d, J=11.20 Hz, 1H), 3.35 (d, J=9.60 Hz, 1H), 3.11-3.30 (m, 4H), 2.93-3.03 (m, 1H), 2.65-2.87 (m, 4H), 1.63-1.98 (m, 4H)).

### (S)-2-(4-(2-(2-(3-fluoro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid (Intermediate ID)

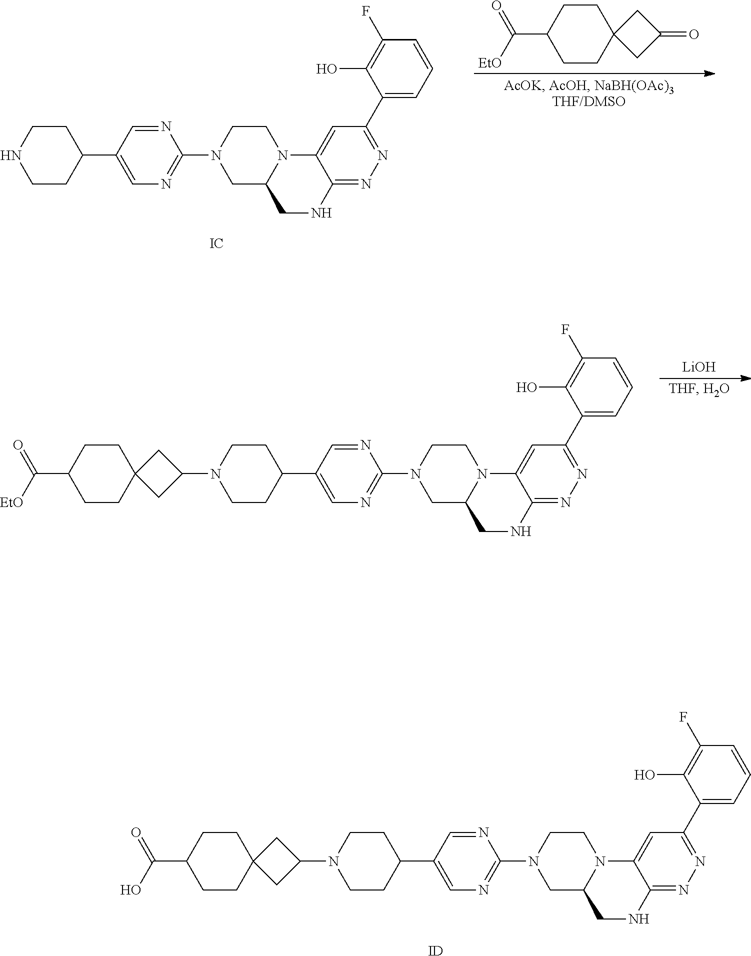

Step 1-(S)-ethyl 2-(4-(2-(2-(3-fluoro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate. To a solution of 2-fluoro-6-[(10S)-12-[5-(4-piperidyl)pyrimidin-2-yl]-1,5,6,8,12-pentazatricyclo[8.4.0.0^{2,7}]tetradeca-2,4,6-trien-4-yl]phenol (440 mg, 951 umol, Intermediate IC) in THF (5 mL), DMSO (1 mL) was added AcOK (280 mg, 2.85 mmol) and the mixture was stirred for 0.5 hour at 50° C. Next, AcOH (171.38 mg, 2.85 mmol, 163.22 uL) and ethyl 2-oxospiro[3.5]nonane-7-carboxylate (500 mg, 2.38 mmol) was added and stirred for another 1.5 hours. Finally, NaBH(OAc)₃ (605 mg, 2.85 mmol) was added at 0° C. and stirred for 12 hrs at 25° C. On completion, the reaction mixture was diluted with water (10 mL) and extracted with DCM (10 mL×3). The combined organic layers were washed with aqueous NaCl 30 mL (10 mL×3), dried over Na₂SO₄, filtered and concentrated under reduced pressure to give a residue. The residue was purified by column chromatography (SiO₂, DCM/MeOH=50/1 to 20/1) to give the title compound (270 mg, 36% yield) as a yellow solid. LC-MS (ESI⁺) m/z 657.6 (M+H)⁺.

Step 2-(S)-2-(4-(2-(2-(3-fluoro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid. To a solution of ethyl 2-[4-[2-[(10S)-4-(3-fluoro-2-hydroxy-phenyl)-1,5,6,8,12-pentazatricyclo[8.4.0.0^{2,7}]tetradeca-2,4,6-trien-12-yl]pyrimidin-5-yl]-1-piperidyl]spiro[3.5]nonane-7-carboxylate (270 mg, 411 umol) in H₂O (2 mL), THF (2 mL), and MeOH (2 mL) was added LiOH·H₂O (103 mg, 2.47 mmol). The mixture was stirred at 25° C. for 2 hrs. On completion, the pH value of the mixture was adjusted to 7 by adding 1 M HCl, and then the mixture was concentrated under reduced pressure to give the title compound (500 mg) as a yellow oil. LC-MS (ESI⁺) m/z 629.4 (M+H)⁺.

### 2-(Trimethylsilyl)ethyl 4-oxopiperidine-1-carboxylate (Intermediate IF)

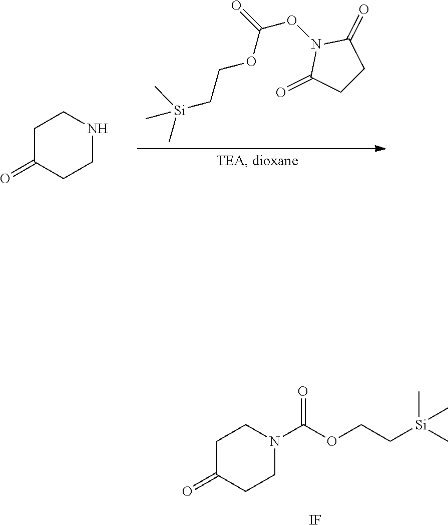 [SMILES: O=C1CCNCC1 ; C[Si](C)(C)CCOC(=O)ON1C(=O)CCC1=O ; C[Si](C)(C)CCOC(=O)N1CCC(=O)CC1 ; "IF"]

To a solution of piperidin-4-one (10 g, 73.7 mmol, CAS #41661-17-6) in dioxane (300 mL) was added TEA (22.4 g, 221 mmol) and (2,5-dioxopyrrolidin-1-yl) 2-trimethylsilylethyl carbonate (19.1 g, 73.7 mmol, CAS #78269-85-9). The mixture was stirred at 25° C. for 12 hours. On completion, the reaction mixture was quenched with water (50 mL) and extracted by DCM (3×20 mL). The extracts were dried over anhydrous sodium sulfate, filtered and concentrated in vacuo to give the crude residue. The residue was purified by column chromatography (SiO2, Petroleum ether/Ethyl acetate=20/1 to 1/1) to give the title compound (12 g, 66% yield) as a white oil. 1H NMR (400 MHz, CHLOROFORM-d) δ ppm 0.06 (s, 9H) 1.01-1.09 (m, 2H) 1.59 (s, 1H) 2.46 (t, J=6.13 Hz, 4H) 3.77 (t, J=6.07 Hz, 4H) 4.20-4.30 (m, 2H).

### (S)-2-(8-(piperidin-4-yl)-6,6a,7,8,9,10-hexahydro-5H-pyrazino[1′,2′:4,5]pyrazino[2,3-c]pyridazin-2-yl)phenol (Intermediate IG)

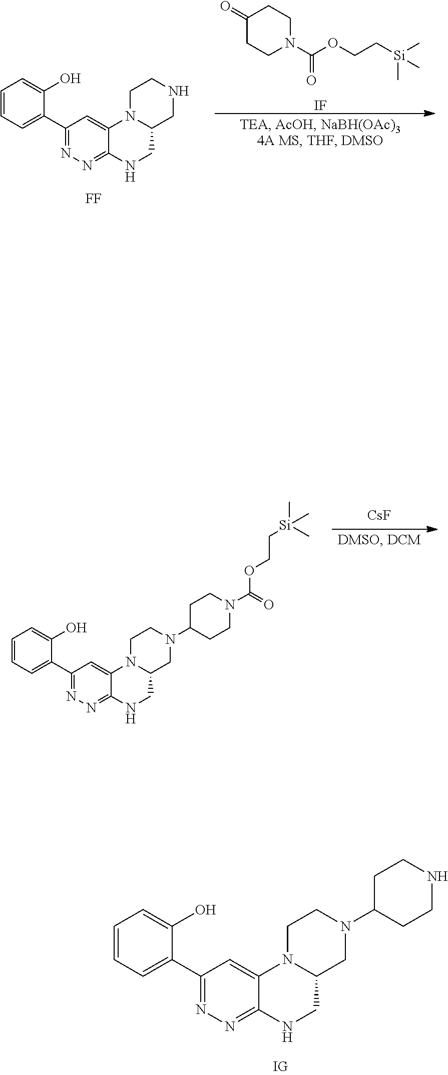

Step 1-(S)-2-(trimethylsilyl)ethyl 4-(2-(2-hydroxyphenyl)-6a,7,9,10-tetrahydro-5H-pyrazino[1′,2′:4,5]pyrazino[2,3-c]pyridazin-8(6H)-yl)piperidine-1-carboxylate. To a solution of 2-[(10R)-1,5,6,8,12-pentazatricyclo[8.4.0.02,7]tetradeca-2,4,6-trien-4-yl]phenol (1.67 g, 4.69 mmol, Intermediate FF) in THF (40 mL) and DMSO (20 mL) was added TEA (949 mg, 9.38 mmol) to adjust the pH to around 7. Then 2-trimethylsilylethyl 4-oxopiperidine-1-carboxylate (2.28 g, 9.38 mmol, Intermediate IF), AcOH (563 mg, 9.38 mmol) and 4 Å molecular sieves (4 g) were added to the mixture which was then stirred at 25° C. for 2 hours. Next, NaBH(OAc)₃ (2.98 g, 14.0 mmol) was added to the mixture at 0° C. Then the mixture was stirred at 25° C. for 12 hours. On completion, the reaction mixture was filtered and the filtrate was quenched with NaHCO₃ (50 mL) at 25° C., and then diluted with DCM (50 mL) and extracted with DCM (50 mL×3). The combined organic layers were washed with NaCl (50 mL×3), dried over Na₂SO₄, filtered and concentrated under reduced pressure to give a residue. The residue was purified by column chromatography (SiO₂, DCM: MeOH=50:1 to 20:1) to give the title compound (1.5 g, 59% yield) as a yellow syrup. LC-MS (ESI⁺) m/z 511.4 (M+H)⁺.

Step 2-(S)-2-(8-(piperidin-4-yl)-6,6a,7,8,9,10-hexahydro-5H-pyrazino[1′,2′:4,5]pyrazino[2,3-c]pyridazin-2-yl)phenol. To a solution of 2-trimethylsilylethyl 4-[(10S)-4-(2-hydroxyphenyl)-1,5,6,8,12-pentazatricyclo[8.4.0.02,7]tetradeca-2,4,6-trien-12-yl]piperidine-1-carboxylate (1.3 g, 2.55 mmol) in DMSO (14 mL) and DCM (3 mL) was added CsF (1.82 g, 11.9 mmol). The mixture was then stirred at 50° C. for 12 hrs. On completion, the mixture was filtered and concentrated under reduced pressure to give a residue. The crude product was purified by reversed-phase HPLC (0.1% FA). After pre-HPLC purification, the eluent was concentrated to remove organic solvents. The residual aqueous solution was lyophilized to give the title compound (0.5 g, 47% yield) as a pink solid. LC-MS (ESI⁺) m/z 367.3 (M+H)⁺.

### Ethyl 2-(3-hydroxyisoxazol-5-yl)-3-methylbutanoate (Intermediate IH)

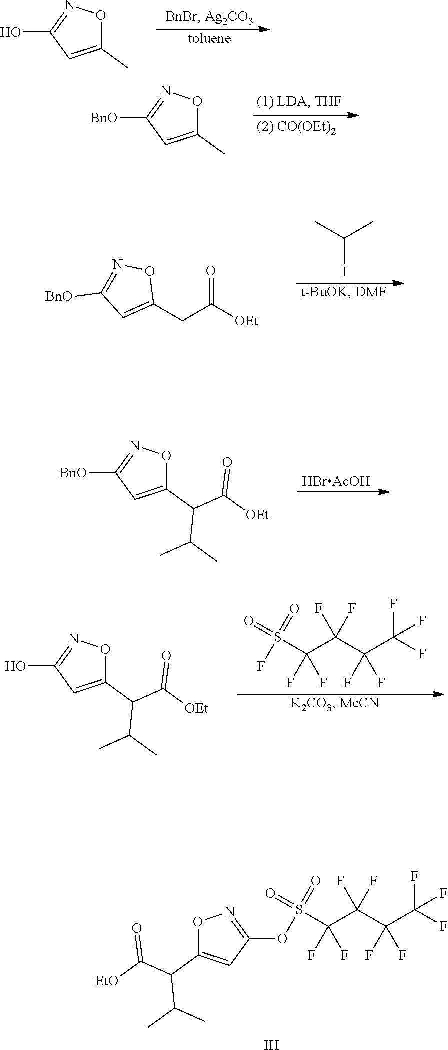

Step 1-3-(Benzyloxy)-5-methylisoxazole. To a solution of 5-methylisoxazol-3-ol (70 g, 0.71 mol, CAS #10004-44-1) in toluene (500 mL) was added BnBr (126 mL, 1.1 mol) and Ag₂CO₃ (273 g, 0.99 mol), then the mixture was stirred at 60° C. for 12 hrs under N₂. On completion, the mixture was filtered and concentrated under reduced pressure to give a residue. The residue was purified by column chromatography (SiO₂, petroleum ether/ethyl acetate=50/1 to 10/1) to give the title compound (85 g, 64% yield) as a colorless oil. LC-MS (ESI, m/z): [M+H]⁺=190.0. ¹H NMR (400 MHz, CDCl₃) δ 7.50-7.32 (m, 5H), 5.66 (d, J=0.6 Hz, 1H), 5.26 (s, 2H), 2.34 (s, 3H).

Step 2-Ethyl 2-(3-(benzyloxy)isoxazol-5-yl)acetate. To a solution of 3-benzyloxy-5-methyl-isoxazole (45 g, 0.24 mol) in THF (800 mL) was added LDA (2 M, 143 mL) at −78° C., then the mixture was stirred at −78° C. for 0.5 hrs. Next, diethyl carbonate (42 g, 0.36 mmol) was added to the mixture, and the mixture was stirred at −78° C. for 2.5 hr. On completion, the reaction mixture was quenched with sat. NH₄Cl (200 mL) at −70° C., and then the mixture was diluted with EA (200 mL) and extracted with EA (500 mL×3). The combined organic layers were washed with sat. brine (500 mL), dried over Na₂SO₄, filtered and concentrated under reduced pressure to give a residue. The residue was purified by column chromatography (SiO₂, petroleum ether/ethyl acetate=I/O to 50/1) to give the title compound (23 g, 88 mmol, 37% yield) as a colorless oil.) as a white solid. ¹H NMR (400 MHz, CDCl₃) δ 7.52-7.32 (m, 5H), 5.95 (s, 1H), 5.27 (s, 2H), 4.22 (q, J=7.2 Hz, 2H), 3.72 (s, 2H), 1.29 (t, J=7.2 Hz, 3H).

Step 3-Ethyl 2-(3-(benzyloxy)isoxazol-5-yl)-3-methylbutanoate. To a solution of ethyl 2-(3-benzyloxyisoxazol-5-yl)acetate (23 g, 88 mmol) in DMF (150 mL) was added t-BuOK (15 g, 0.13 mol) at 0° C. Then 2-iodopropane (16 g, 92 mmol, CAS #75-30-9) was added to the mixture, and the mixture was stirred at 0° C. for 2 hrs. On completion, the reaction mixture was quenched with sat. NH₄Cl (100 mL) at 0° C., and then diluted with EA (200 mL) and extracted with EA (200 mL×3). The combined organic layers were washed with sat. brine (200 mL), dried over Na₂SO₄, filtered and concentrated under reduced pressure to give the title compound (26 g) as a yellow oil.

Step 4-Ethyl 2-(3-hydroxyisoxazol-5-yl)-3-methylbutanoate. A solution of ethyl 2-(3-benzyloxyisoxazol-5-yl)-3-methyl-butanoate (23 g, 76 mmol) in HBrHOAc (89 g, 0.33 mol, 30% solution) was stirred at 25° C. for 16 hrs. On completion, the reaction mixture was quenched with sat. NH₄Cl (80 mL), and then diluted with EA (50 mL) and extracted with EA mL (100 mL×3). The combined organic layers were washed with sat. brine (100 mL), dried over Na₂SO₄, filtered and concentrated under reduced pressure to give a residue. The residue was purified by column chromatography (SiO₂, petroleum ether/ethyl acetate=20/1 to 5/1) to give the title compound (14 g, 87% yield) as a yellow oil. LC-MS (ESI, m/z): [M+H]⁺=214.2. H NMR (400 MHz, CDCl₃) δ 5.95 (s, 1H), 4.28-4.12 (m, 2H), 3.45 (d, J=8.8 Hz, 1H), 1.28 (t, J=7.2 Hz, 3H), 1.02 (d, J=6.8 Hz, 3H), 0.93 (d, J=6.8 Hz, 3H).

Step 5-Ethyl 3-methyl-2-(3-(((perfluorobutyl)sulfonyl)oxy)isoxazol-5-yl)butanoate. To a solution of ethyl 2-(3-hydroxyisoxazol-5-yl)-3-methyl-butanoate (3 g, 14.1 mmol) in MeCN (20 mL) was added 1,1,2,2,3,3,4,4,4-nonafluorobutane-1-sulfonyl fluoride (4.68 g, 15.5 mmol, CAS #375-72-4) and K₂CO₃ (3.89 g, 28.1 mmol). The mixture was stirred at 40° C. for 12 hours. On completion, the mixture was concentrated under reduced pressure to give a residue. The residue was purified by column chromatography (SiO₂, PE/EA=1/0 to 10/1) to give the title compound (5.2 g, 71% yield) as a colorless oil. LC-MS (ESI, m/z): [M+H]⁺=496.0.

### (S)-2-chloro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate II) and (R)-2-chloro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (Intermediate IJ)

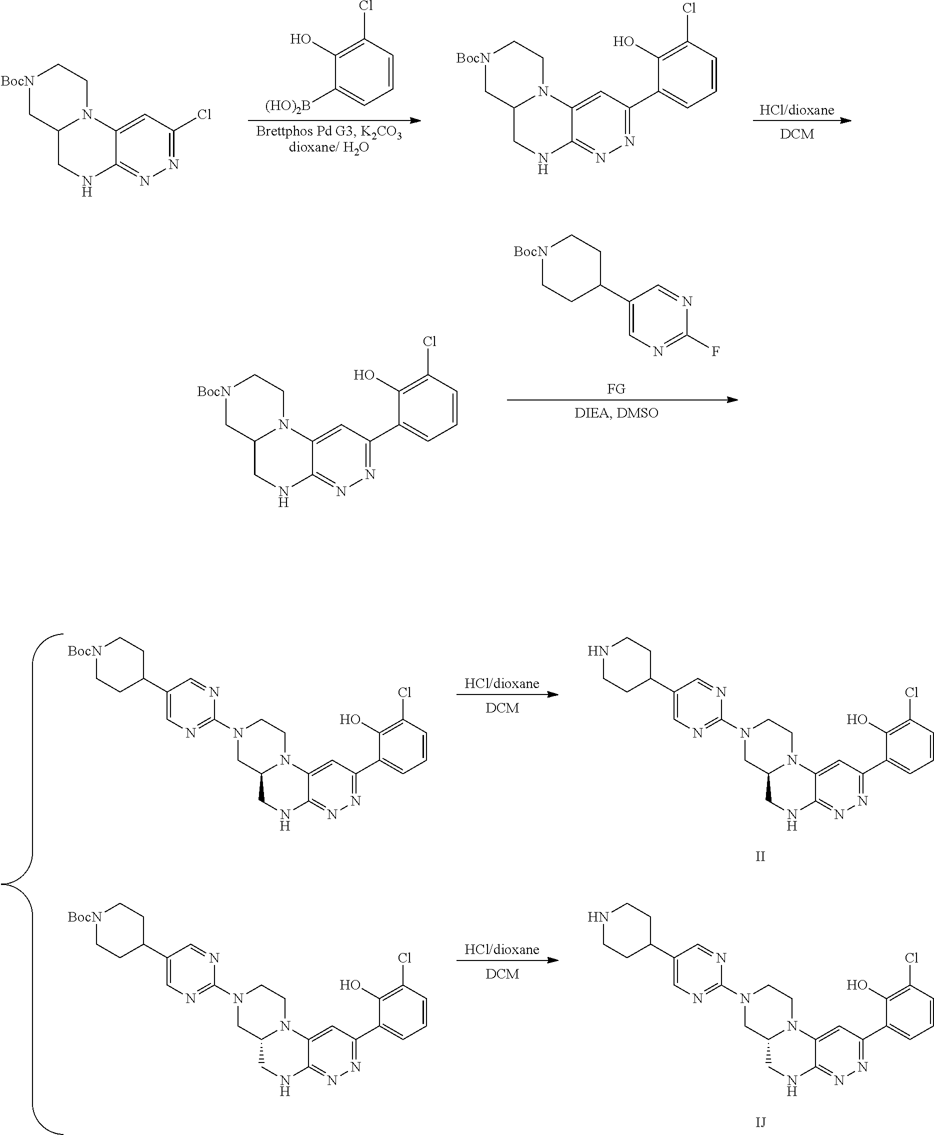

Step 1-Tert-butyl 2-(3-chloro-2-hydroxyphenyl)-6a,7,9,10-tetrahydro-5H-pyrazino[1′,2′:4,5]pyrazino[2,3-c]pyridazine-8(6H)-carboxylate. A mixture of tert-butyl 2-chloro-6a,7,9,10-tetrahydro-5H-pyrazino[1′,2′:4,5]pyrazino[2,3-c]pyridazine-8(6H)-carboxylate (1 g, 3 mmol, synthesized via Steps 1-2 of Intermediate FD), (3-chloro-2-hydroxyphenyl)boronic acid (793 mg, 4.6 mmol, CAS #951655-50-8), [2-(2-aminophenyl)phenyl]-methylsulfonyloxypalladium;dicyclohexyl-[3,6-dimethoxy-2-(2,4,6-triisopropylphenyl)phenyl]phosphane (278 mg, 306 umol), and K₂CO₃ (1.27 g, 9.21 mmol) in dioxane (30 mL) and H₂O (6 mL) was degassed and purged with N₂ three times. Then the mixture was stirred at 100° C. for 12 hours under N₂ atmosphere. On completion, the reaction mixture was concentrated under reduced pressure to remove solvent. The residue was purified by column chromatography (SiO₂, Petroleum ether/Ethyl acetate=5/1 to 0/1) to give the title compound (600 mg, 47% yield) as a yellow solid. LC-MS (ESI⁺) m/z 418.3 (M+H) *.

Step 2-2-chloro-6-(6,6a,7,8,9,10-hexahydro-5H-pyrazino[1′,2′:4,5]pyrazino[2,3-c]pyridazin-2-yl)phenol. A mixture of tert-butyl 2-(3-chloro-2-hydroxyphenyl)-6a,7,9,10-tetrahydro-5H-pyrazino[1′,2′:4,5]pyrazino[2,3-c]pyridazine-8(6H)-carboxylate (600 mg, 1.44 mmol), HCl/dioxane (8 M, 1 ml), in DCM (10 mL) was stirred at 25° C. for 2 hours under N₂ atmosphere. On completion, the reaction mixture was concentrated under reduced pressure to give the title compound (550 mg). LC-MS (ESI⁺) m/z 318.1 (M+H)⁺.

Step 3-(S)-tert-butyl 4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate and (R)-tert-butyl 4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate. A mixture of 2-chloro-6-(6,6a,7,8,9,10-hexahydro-5H-pyrazino[1′,2′:4,5]pyrazino[2,3-c]pyridazin-2-yl)phenol (500 mg, 1.41 mmol), tert-butyl 4-(2-fluoropyrimidin-5-yl)piperidine-1-carboxylate (330 mg, 1.18 mmol, Intermediate FG), and DIEA (760 mg, 5.88 mmol, 1.02 mL) in DMSO (10 mL) was degassed and purged with N₂ three times. Then the mixture was stirred at 80° C. for 2 hours under N₂ atmosphere. On completion, the reaction mixture was quenched with H₂O (10 mL) at 25° C. The mixture was stirred, then filter cake was obtained by vacuum pumping. The residue was purified by prep-HPLC (column: Phenomenex Luna C18 150*25 mm*10 um;mobile phase: [water(0.225% FA)-ACN];B %: 33%-63%,10 min) to give the mixed compound. The mixed compound was further separated by SFC (column: DAICEL CHIRALPAK AD(250 mm*50 mm,10 um);mobile phase:[0.1% NH₃H₂O IPA];B %: 70%-70%,3.5; 60 min) to give (S)-tert-butyl 4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (330 mg, 48% yield) as yellow solid (LC-MS (ESI+) m/z 579.2 (M+H)⁺) and (R)-tert-butyl 4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (230 mg, 33% yield) as yellow solid (LC-MS (ESI+) m/z 579.2 (M+H)⁺). The absolute stereochemistry of the enantiomers was assigned arbitrarily.

Step 4-(S)-2-chloro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol. To a solution of (S)-tert-butyl 4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (50 mg, 86.3 umol) in DCM (4 mL) was added HCl/dioxane (8 M, 32.4 uL). The mixture was stirred at 25° C. for 2 hours. On completion, the reaction mixture was concentrated under reduced pressure to remove the solvent. The residue was purified by prep-HPLC (Phenomenex Luna C18 150*25 mm*10 um;mobile phase: [water(FA)-ACN];B %: 1%-29%,10 min) to give the title compound (22 mg, 53% yield) as a yellow solid. ¹H NMR (400 MHz, DMSO-d₆) δ=8.38 (s, 1H), 8.34 (s, 2H), 7.97 (dd, J=1.2, 8.0 Hz, 1H), 7.57 (d, J=3.2 Hz, 1H), 7.41-7.34 (m, 2H), 6.85 (t, J=8.0 Hz, 1H), 4.72 (t, J=9.2 Hz, 2H), 4.23 (d, J=12.4 Hz, 1H), 3.66 (d, J=3.6 Hz, 1H), 3.62 (s, 1H), 3.38-3.36 (m, 1H), 3.30-3.27 (m, 1H), 3.19 (dd, J=3.2, 12.4 Hz, 3H), 3.00 (dt, J=3.2, 12.4 Hz, 1H), 2.82-2.72 (m, 3H), 2.69-2.60 (m, 1H), 1.84-1.75 (m, 2H), 1.67 (dq, J=3.8, 12.8 Hz, 2H). LC-MS (ESI+) m/z 479.2 (M+H)⁺.

Step 5-(R)-2-chloro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol. To a solution of (R)-tert-butyl 4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (50 mg, 86.3 umol) in DCM (4 mL) was added HCl/dioxane (8 M, 32.4 uL). The mixture was stirred at 25° C. for 2 hours. On completion, the reaction mixture was concentrated under reduced pressure to remove the solvent. The residue was purified by prep-HPLC (Phenomenex Luna C18 150*25 mm*10 um;mobile phase: [water(FA)-ACN];B %: 1%-29%,10 min) to give the title compound (22 mg, 53% yield) as a yellow solid. ¹H NMR (400 MHz, DMSO-d₆) δ=8.38 (s, 1H), 8.34 (s, 2H), 7.97 (dd, J=1.6, 8.4 Hz, 1H), 7.57 (d, J=3.2 Hz, 1H), 7.40-7.34 (m, 2H), 6.85 (t, J=8.0 Hz, 1H), 4.79-4.65 (m, 2H), 4.23 (d, J=12.8 Hz, 1H), 3.66-3.64 (m, 1H), 3.63 (d, J=3.6 Hz, 1H), 3.35 (s, 1H), 3.27 (d, J=2.8 Hz, 1H), 3.17 (d, J=12.4 Hz, 3H), 3.00 (dt, J=3.2, 12.4 Hz, 1H), 2.82-2.70 (m, 3H), 2.62 (tt, J=3.6, 12.0 Hz, 1H), 1.82-1.73 (m, 2H), 1.65 (dq, J=3.2, 12.8 Hz, 2H). LC-MS (ESI+) m/z 479.1 (M+H)⁺.

### (S)-2-(4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid (Intermediate IK)

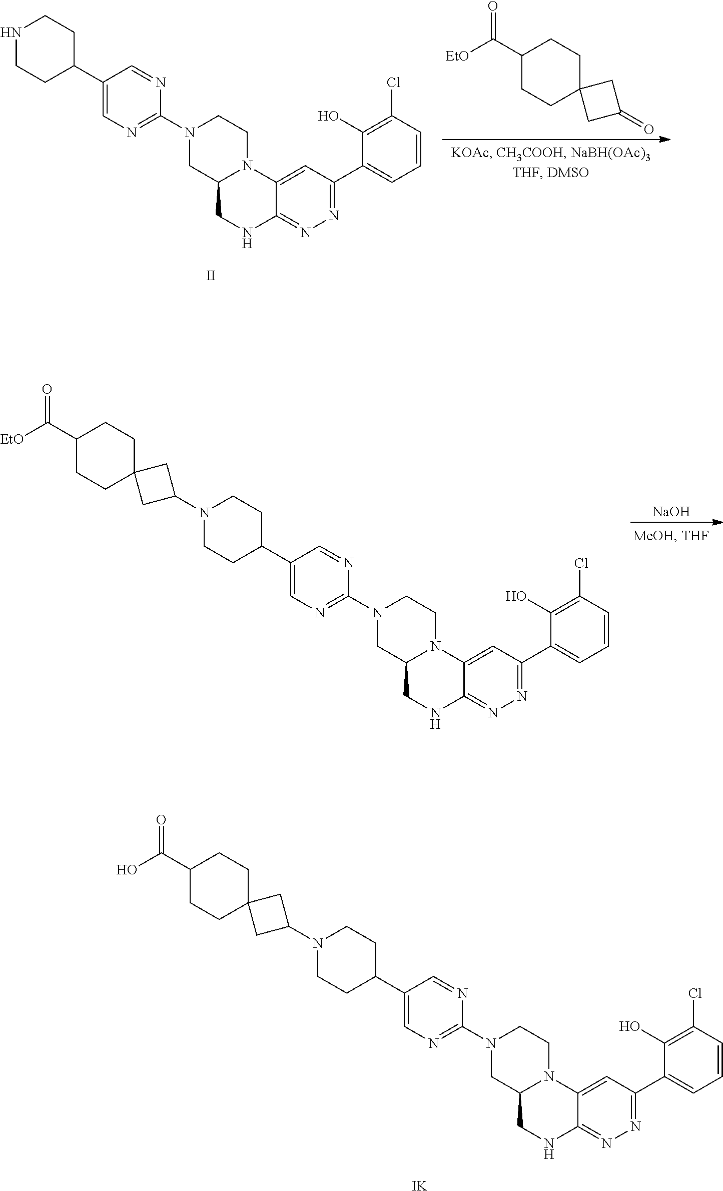

Step 1-(S)-ethyl 2-(4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate. To a solution of (S)-2-chloro-6-(8-(5-(piperidin-4-yl)pyrimidin-2-yl)-6,6a,7,8,9,10-hexahydro-5H-pyrazino[1′,2′:4,5]pyrazino[2,3-c]pyridazin-2-yl)phenol (180 mg, 349 umol, Intermediate II), ethyl 2-oxospiro[3.5]nonane-7-carboxylate (73.4 mg, 349 umol), and KOAc (172 mg, 1.75 mmol) in THF (3 mL) and DMSO (3 mL) was added AcOH (62.9 mg, 1.05 mmol) and NaBH(OAc)₃ (222 mg, 1.05 mmol) at 0° C. The mixture was then stirred at 25° C. for 12 hours. On completion, the reaction mixture was quenched with H₂O (2 mL) at 25° C. The reaction mixture was purified by prep-HPLC (FA condition) to give the title compound (120 mg, 51% yield) as a white solid. LC-MS (ESI+) m/z 673.3 (M+H)⁺.

Step 2-(S)-2-(4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid. To a solution of (S)-ethyl 2-(4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate (120 mg, 178 umol) in MeOH (2 mL) and THF (2 mL) was added NaOH (1 M, 2 mL). The mixture was stirred at 25° C. for 2 hours. On completion, the reaction mixture was made neutral by addition of HCl at 25° C., then concentrated under reduced pressure to give the title compound (110 mg) as a white solid. LC-MS (ESI+) m/z 645.5 (M+H)⁺.

### (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (CAS #1948273-03-7) (Intermediate CF)

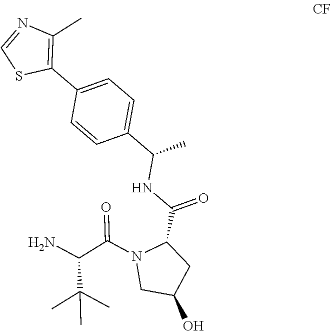 [SMILES: Cc1ncsc1-c1ccc([C@H](C)NC(=O)[C@@H]2C[C@@H](O)CN2C(=O)[C@@H](N)C(C)(C)C)cc1]

### (2S)-2-(1-tert-butoxycarbonyl-4-piperidyl)-2-(9H-fluoren-9-ylmethoxycarbonylamino)acetic acid (Intermediate IO)

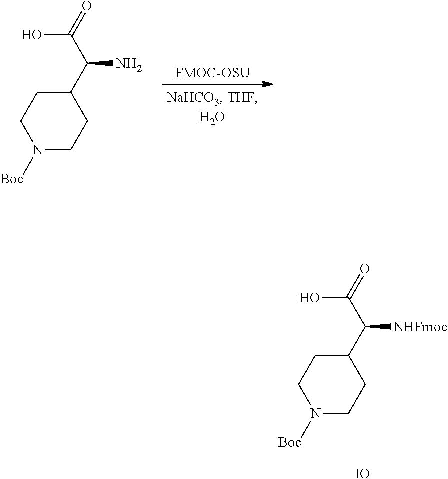

To a solution of (2S)-2-amino-2-(1-tert-butoxycarbonyl-4-piperidyl)acetic acid (200 mg, 774 umol, CAS #368866-11-9) in H₂O (3 mL) was added to NaHCO₃ (130 mg, 1.55 mmol), then FMOC-OSU (287 mg, 851 umol, CAS #82911-69-1) and THF (4.8 mL) was dropwise added the mixture and stirred at 25° C. for 5 hours. On completion, the reaction mixture was quenched with NH₄Cl (10 mL), and then extracted with EtOAc (10 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=5/1 to 0/1) to give the title compound (600 mg, 79% yield) as a white solid. ¹H NMR (400 MHz, DMSO-d6) δ=13.12-12.30 (m, 1H), 7.89 (d, J=7.6 Hz, 2H), 7.74 (d, J=7.6 Hz, 2H), 7.67 (d, J=8.6 Hz, 1H), 7.41 (t, J=7.6 Hz, 2H), 7.36-7.28 (m, 2H), 4.33-4.25 (m, 2H), 4.22 (d, J=7.2 Hz, 1H), 4.06-3.99 (m, 2H), 3.98-3.92 (m, 2H), 3.82-3.62 (m, 1H), 2.59 (s, 1H), 1.53 (dd, J=13.6, 18.4 Hz, 2H), 1.39 (s, 9H), 1.20-1.15 (m, 2H). LC-MS (ESI⁺) m/z 526.3 (M+H)⁺.

### Tert-butyl 4-[(1S)-1-amino-2-[(2S,4R)-2-[(4-ethynylphenyl)methylcarbamoyl]-4-hydroxy-pyrrolidin-1-yl]-2-oxo-ethyl]piperidine-1-carboxylate (Intermediate IP)

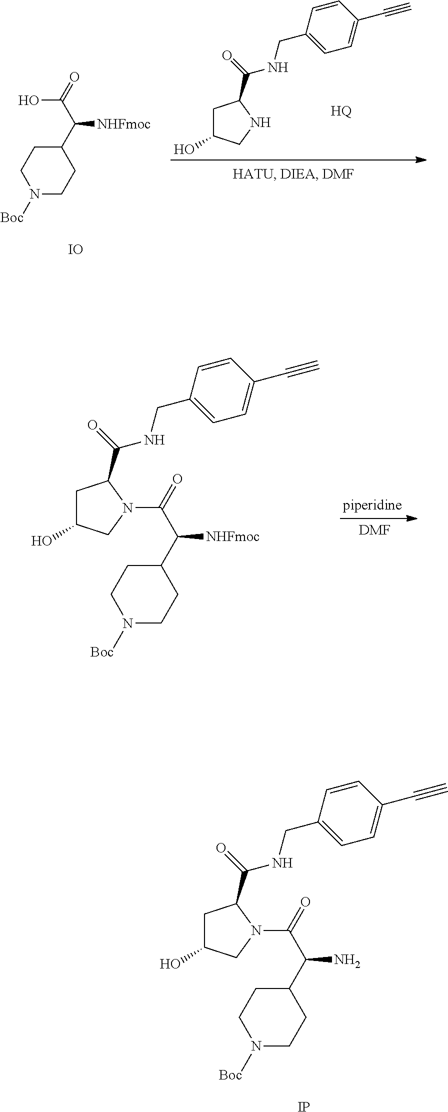

Step 1-Tert-butyl 4-[(1S)-2-[(2S,4R)-2-[(4-ethynylphenyl)methylcarbamoyl]-4-hydroxy-pyrrolidin-1-yl]-1-(9H-fluoren-9-ylmethoxycarbonylamino)-2-oxo-ethyl]piperidine-1-carboxylate. To a solution of (2S,4R)—N-[(4-ethynylphenyl)methyl]-4-hydroxy-pyrrolidine-2-carboxamide (90.0 mg, 368 umol, Intermediate HQ) and HATU (168 mg, 442 umol) in DMF (1 mL) was stirred at 25° C. for 30 minutes. Then (2S)-2-(1-tert-butoxycarbonyl-4-piperidyl)-2-(9H-fluoren-9-ylmethoxycarbonylamino)acetic acid (265 mg, 552 umol, Intermediate IO) and DIEA (142 mg, 1.11 mmol) was added, and the mixture was stirred at 25° C. for 2 hours. On completion, the reaction mixture was quenched with sat. NH₄Cl (1 mL), and then extracted with EtOAc (5 mL×3). The combined organic layers were washed with brine (5 mL×3), dried over Na₂SO₄, filtered and concentrated under reduced pressure to give a residue. The residue was purified by column chromatography (SiO₂, Petroleum ether/Ethyl acetate=0/1) to give the title compound (200 mg, 72% yield) as a yellow oil. ¹H NMR (400 MHz, DMSO-d6) δ=8.49 (t, J=5.2 Hz, 1H), 7.95 (s, 1H), 7.89 (d, J=7.6 Hz, 2H), 7.74 (t, J=6.8 Hz, 2H), 7.62 (d, J=8.0 Hz, 1H), 7.44-7.38 (m, 4H), 7.35-7.30 (m, 2H), 7.28 (d, J=8.0 Hz, 2H), 5.12 (d, J=3.6 Hz, 1H), 4.40 (t, J=7.6 Hz, 1H), 4.37-4.31 (m, 2H), 4.26 (s, 3H), 4.21 (d, J=5.6 Hz, 2H), 4.17-4.11 (m, 2H), 3.61 (d, J=10.0 Hz, 2H), 3.17 (d, J=5.2 Hz, 2H), 2.89 (s, 2H), 2.73 (s, 3H), 1.73-1.59 (m, 2H), 1.45 (s, 2H), 1.38 (s, 9H). LC-MS (ESI⁺) m/z 707.4 (M+H)⁺.

Step 2-Tert-butyl 4-[(1S)-1-amino-2-[(2S,4R)-2-[(4-ethynylphenyl)methylcarbamoyl]-4-hydroxy-pyrrolidin-1-yl]-2-oxo-ethyl]piperidine-1-carboxylate. To a solution of tert-butyl 4-[(1S)-2-[(2S,4R)-2-[(4-ethynylphenyl)methylcarbamoyl]-4-hydroxy-pyrrolidin-1-yl]-1-(9H-fluoren-9-ylmethoxycarbonylamino)-2-oxo-ethyl]piperidine-1-carboxylate (200 mg, 282 umol) and piperidine (862 mg, 10.1 mmol) in DMF (4 mL) was stirred at 25° C. for 30 minutes. On completion, the reaction mixture was concentrated in vacuo to give a residue. The crude product was purified by reversed-phase HPLC(0.1% NH₃•H₂O) to give the title compound (100 mg, 65% yield) as a brown oil. LC-MS (ESI⁺) m z 485.1 (M+H)⁺.

### (R)-2-(4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid (Intermediate IQ)

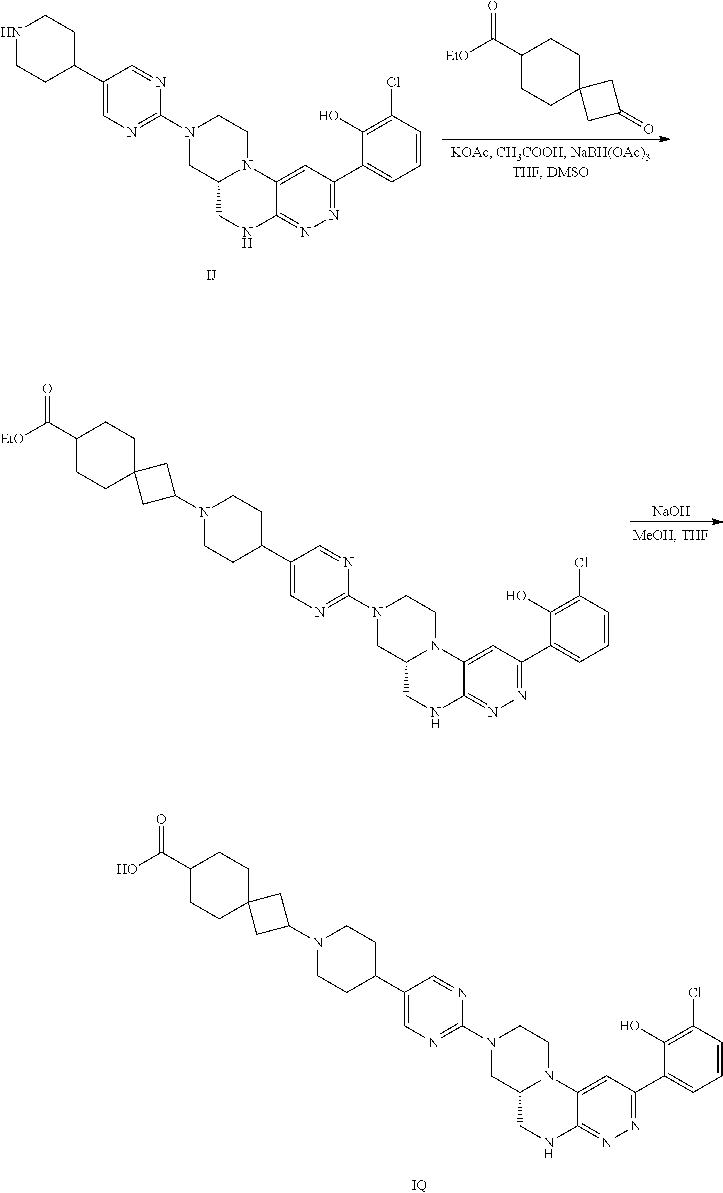

Step 1-(R)-ethyl 2-(4-(2-(2-(3-chloro-2-hydroxyphenyl)-6a,7,9,10-tetrahydro-5H-pyrazino[1′,2′: 4,5]pyrazino[2,3-c]pyridazin-8(6H)-yl)pyrimidin-5-yl)piperidin-1-yl)spiro[3.5]nonane-7-carboxylate. To a solution of (R)-tert-butyl 4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidine-1-carboxylate (100 mg, 194 umol, HCl, Intermediate IJ) and ethyl 2-oxospiro[3.5]nonane-7-carboxylate (61.2 mg, 291 umol, CAS #1615656-09-1) in THF (2 mL) and DMSO (2 mL) was added AcOK (95.2 mg, 970 umol) and AcOH (34.9 mg, 582 umol). The mixture was stirred at 25° C. for 1 hr. Then NaBH(OAc)₃ (123 mg, 582 umol) was added to the reaction mixture at 0° C. Then the mixture was stirred at 25° C. for 15 hrs. On completion, the reaction mixture was quenched with H₂O (2 mL) at 0° C. The residue was purified by prep-HPLC (FA condition) to give the title compound (90 mg, 66% yield) as a white solid. LC-MS (ESI⁺) m/z 673.3 (M+H)⁺.

Step 2-(R)-2-(4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid. To a solution of ethyl (R)-ethyl 2-(4-(2-(2-(3-chloro-2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate (90.0 mg, 133 umol) in THF (1 mL) and MeOH (1 mL) was added NaOH (1 M, 1.80 mL). The mixture was stirred at 25° C. for 1 hour. On completion, the reaction mixture was concentrated under reduced pressure to remove solvent to give the title compound (200 mg) as a white solid. LC-MS (ESI⁺) m/z 645.3 (M+H)⁺.

### Tert-butyl 6-(4-(2-fluoropyrimidin-5-yl)piperidin-1-yl)-2-azaspiro[3.3]heptane-2-carboxylate (Intermediate IR)

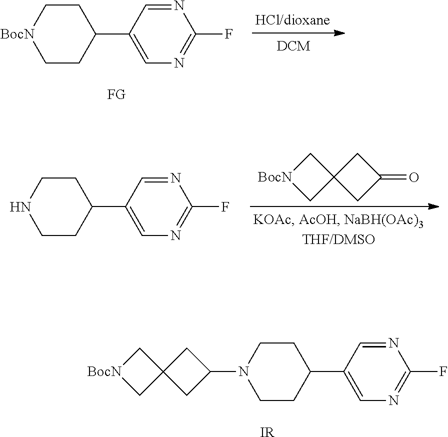

Step 1-2-Fluoro-5-(piperidin-4-yl)pyrimidine. To a solution of tert-butyl 4-(2-fluoropyrimidin-5-yl)piperidine-1-carboxylate (24 g, 85 mmol, Intermediate FG) in DCM (240 mL) was added HCl/dioxane (4 M, 96 mL). The mixture was stirred at 25° C. for 2 hr. On completion, the residue was concentrated in vacuo to give the title compound (1.48 g, 57% yield, FA) as a white solid. LC-MS (ESI⁺) m/z 182.2(M+H)⁺.

Step 2-Tert-butyl 6-(4-(2-fluoropyrimidin-5-yl)piperidin-1-yl)-2-azaspiro[3.3]heptane-2-carboxylate. To a solution of 2-fluoro-5-(4-piperidyl)pyrimidine (8.2 g, 37.7 mmol, HCl) in THF (120 mL) and DCE (30 mL) was added TEA (3.81 g, 37.7 mmol) and tert-butyl 6-oxo-2-azaspiro[3.3]heptane-2-carboxylate (7.96 g, 37.7 mmol). After stirred at 25° C. for 1 hr, NaBH(OAc)₃ (23.95 g, 113.01 mmol) was added at 0° C. Then the mixture was stirred at 25° C. for 12 hrs. On completion, the reaction was diluted with DCM (200 mL) and saturated NaHCO₃ (50 mL, aq.) and extracted with DCM (50 mL×3). The combined organic layers were dried over Na₂SO₄, filtered and concentrated under reduced pressure to give a residue. The residue was purified by column chromatography (SiO2, Dichloromethane Methanol=I/O to 10/1) to give the title compound (19 g, 63% yield) as a white solid. LC-MS (ESI⁺) m z 377.1(M+H)⁺.

### 3-(2-(Aminomethyl)-5-ethynylphenoxy)-N,N-dimethylpropan-1-amine (Intermediate IS)

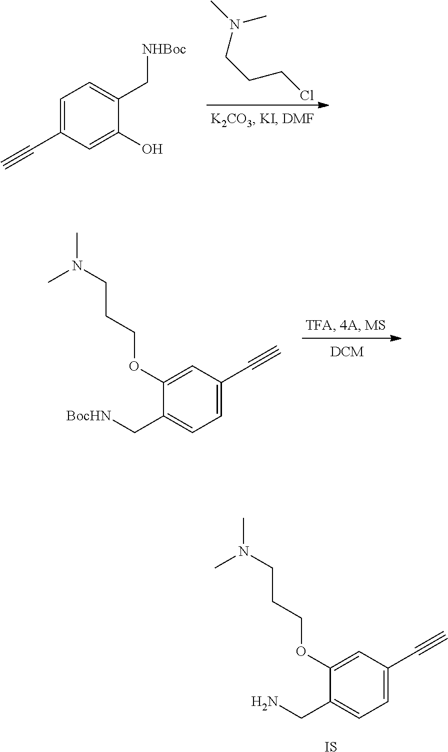

Step 1-Tert-butyl 2-(3-(dimethylamino)propoxy)-4-ethynylbenzylcarbamate. To a solution of tert-butyl N-[(4-ethynyl-2-hydroxy-phenyl)methyl]carbamate (1.4 g, 5.7 mmol, synthesized via Steps 1-3 of Intermediate HY) in DMF (25 mL) was added K₂CO₃ (2.35 g, 17.0 mmol), KI (188 mg, 1.13 mmol) and 3-chloro-N,N-dimethyl-propan-1-amine (1.03 g, 8.49 mmol, CAS #109-54-6). The mixture was stirred at 70° C. for 2 hours. On completion, the reaction mixture was quenched with saturated NH₄Cl aqueous solution 30 mL at 25° C., and then diluted with EA (20 mL) and extracted with EA (20 mL×3). The combined organic layers were washed with sat NaCl (10 mL×3), dried over Na₂SO₄ filtered and concentrated under reduced pressure to give a residue. The crude product was purified by column chromatography (SiO₂, DCM/MEOH=100/1 to 50/1) to give title compound (1.14 g, 53% yield) as a brown solid. ¹H NMR (400 MHz, DMSO-d₆) δ 7.22 (t, J=5.6 Hz, 1H), 7.14-7.09 (m, 1H), 7.05-7.00 (m, 1H), 4.15-3.95 (m, 5H), 3.37 (s, 1H), 2.37 (t, J=6.8 Hz, 2H), 2.14 (s, 6H), 1.85 (t, J=6.4 Hz, 2H), 1.41-1.36 (m, 9H); LC-MS (ESI⁺) m/z 333.5 (M+H)⁺

Step 2-3-(2-(Aminomethyl)-5-ethynylphenoxy)-N,N-dimethylpropan-1-amine. To a solution of tert-butyl N-[[2-[3-(dimethylamino)propoxy]-4-ethynyl-phenyl]methyl]carbamate (900 mg, 3 mmol) in DCM (9 mL) was added TFA (5.63 g, 49.4 mmol, 3.66 mL) and 4 Å molecular sieves (0.9 g, 3 mmol). The mixture was stirred at 25° C. for 1 hour. On completion, the resulting product was dissolved in DCM (9 mL) and filtered to remove the insoluble. The filtrate was concentrated under reduced pressure to the title compound (2.36 g, TFA salt) as a brown oily substance. LC-MS (ESI⁺) m/z 216.1(M−NH₂+H)⁺.

### (2S,4R)-1-((S)-2-amino-3,3-dimethylbutanoyl)-N-(4-ethynyl-2-(3-(methylamino)propoxy)benzyl)-4-hydroxypyrrolidine-2-carboxamide (Intermediate IT)

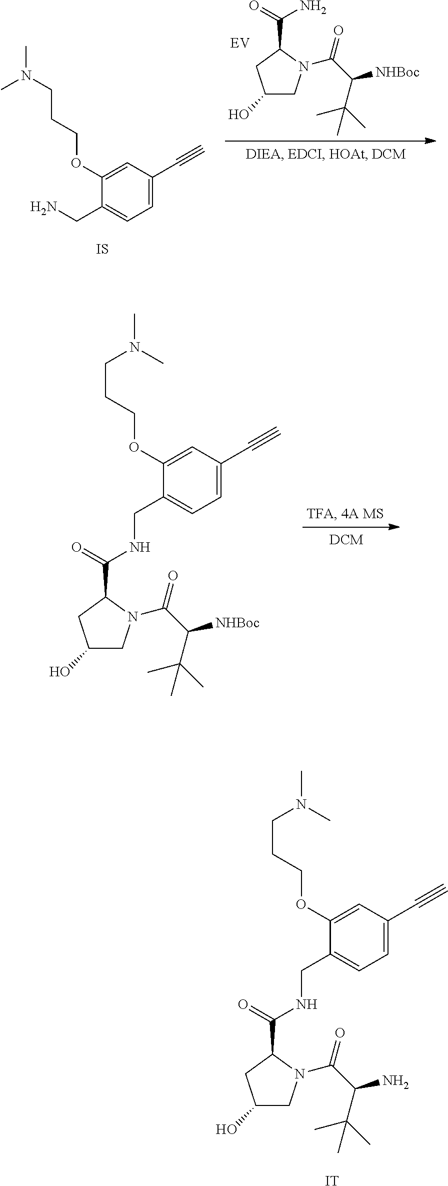

Step 1-Tert-butyl ((S)-1-((2S,4R)-2-((2-(3-(dimethylamino)propoxy)-4-ethynylbenzyl)carbamoyl)-4-hydroxypyrrolidin-1-yl)-3,3-dimethyl-1-oxobutan-2-yl)carbamate. To a solution of (2S,4R)-1-[2-(tert-butoxycarbonylamino)-3,3-dimethyl-butanoyl]-4-hydroxy-pyrrolidine-2-carboxylic acid (2.35 g, 6.82 mmol. Intermediate EV) in DCM (45 mL) was added DIEA (4.41 g, 34.1 mmol, 5.94 mL), EDCI (1.57 g, 8.19 mmol) and HOAt (1.11 g, 8.19 mmol, 1.15 mL). After 10 minutes, 3-[2-(aminomethyl)-5-ethynyl-phenoxy]-N,N-dimethyl-propan-1-amine (2.36 g, 6.82 mmol, TFA, Intermediate IS) was added and the mixture was stirred at 25° C. for 12 hours. On completion, the reaction mixture was concentrated under reduced pressure to remove solvent. The crude product was purified by reversed-phase HPLC (0.1% FA condition) to give the title compound (950 mg, 24% yield) as a brown oily substance. ¹H NMR (400 MHz, DMSO-d₆) δ=8.46-8.40 (m, 1H), 8.19 (s, 1H), 7.33 (d, J=7.6 Hz, 1H), 6.99 (s, 1H), 6.93 (d, J=7.6 Hz, 1H), 6.46 (d, J=9.2 Hz, 1H), 5.75 (s, 1H), 4.45 (t, J=8.4 Hz, 1H), 4.34 (s, 1H), 4.29-4.21 (m, 1H), 4.16-4.10 (m, 2H), 4.03 (t, J=6.4 Hz, 2H), 3.67-3.55 (m, 5H), 2.23 (s, 5H), 2.09-1.97 (m, 1H), 1.88 (td, J=6.5, 13.6 Hz, 3H), 1.38 (s, 9H), 0.91 (s, 9H); LC-MS (ESI⁺) m/z559.5 (M+H)⁺.

Step 2-(2S,4R)-1-((S)-2-amino-3,3-dimethylbutanoyl)-N-(4-ethynyl-2-(3-(methylamino)propoxy)benzyl)-4-hydroxypyrrolidine-2-carboxamide. To a solution of tert-butyl N-[1-[(2S,4R)-2-[[2-[3-(dimethylamino)propoxy]-4-ethynyl-phenyl]methylcarbamoyl]-4-hydroxy-pyrrolidine-1-carbonyl]-2,2-dimethyl-propyl]carbamate (750 mg, 1.34 mmol) in DCM (8 mL) was added TFA (2.46 g, 21.61 mmol, 1.60 mL) and 4 Å molecular sieves (750 mg, 1.34 mmol). The mixture was stirred at 25° C. for 1 hour. On completion, the resulting product was dissolved in DCM (8 mL) and filtered to remove the insoluble and concentrated under reduced pressure to give a residue. The crude product was purified by reversed-phase HPLC (0.1% NH₃•H₂O) to give title compound (350 mg, 44% yield) as a brown oily substance. ¹H NMR (400 MHz, DMSO-d6) δ=8.38 (t, J=6.0 Hz, 1H), 7.33 (d, J=7.6 Hz, 1H), 7.14 (d, J=7.6 Hz, 1H), 7.05-6.96 (m, 1H), 6.93 (d, J=7.6 Hz, 1H), 5.75 (s, 1H), 5.16-4.92 (m, 1H), 4.46 (t, J=8.0 Hz, 1H), 4.35 (s, 1H), 4.28-4.20 (m, 1H), 4.18-4.09 (m, 2H), 4.02 (t, J=6.0 Hz, 2H), 3.62-3.51 (m, 2H), 3.23 (s, 1H), 2.38 (t, J=7.2 Hz, 2H), 2.15 (s, 6H), 2.06-1.97 (m, 1H), 1.95-1.81 (m, 3H), 0.89 (s, 9H) LC-MS (ESI⁺) m/z459.4 (M+H)⁺

### Phenyl ((S)-1-((2S,4R)-2-((2-(3-(dimethylamino)propoxy)-4-ethynylbenzyl)carbamoyl)-4-hydroxypyrrolidin-1-yl)-3,3-dimethyl-1-oxobutan-2-yl)carbamate (Intermediate IU)

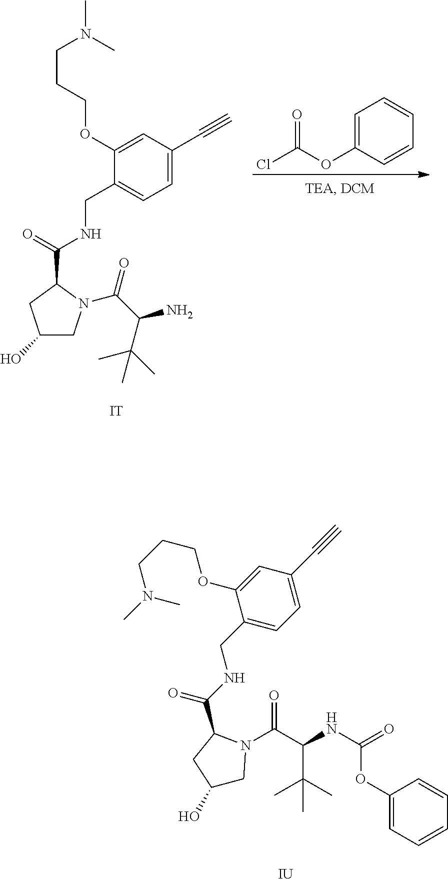

To a solution of (2S,4R)-1-[(2S)-2-amino-3,3-dimethyl-butanoyl]-N-[[2-[3-(dimethylamino)propoxy]-4-ethynyl-phenyl]methyl]-4-hydroxy-pyrrolidine-2-carboxamide (120 mg, 210 umol, TFA) in DCM (1 mL) was added TEA (63.62 mg, 628.7 umol) phenyl carbonochloridate (39.37 mg, 251.48 umol, CAS #1885-14-9). The mixture was stirred at 0° C. for 0.5 hour. On completion, the reaction mixture was quenched with sat. NH₄Cl (5 mL) at 25° C., and then filtered and concentrated under reduced pressure to give the title compound (200 mg) as a white solid. LC-MS (ESI⁺) m/z 579.5 (M+H)⁺.

### 5-Ethyl-3-(2-(methoxymethoxy)phenyl)-6,7,8,9-tetrahydro-5H-pyrido[3′,4′0.4,5]pyrrolo[2,3-c]pyridazine (Intermediate IV)

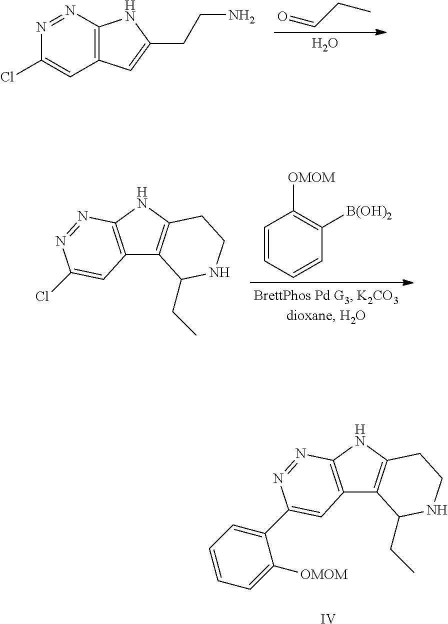

Step 1-3-chloro-5-ethyl-6,7,8,9-tetrahydro-5H-pyrido[3′,4′:4,5]pyrrolo[2,3-c]pyridazine. To a solution of 2-(3-chloro-7H-pyrrolo[2,3-c]pyridazin-6-yl)ethanamine (2 g, 10 mmol, synthesized via Steps 1-3 of Intermediate Y) in H₂O (20 mL) was added propanal (1.77 g, 30.5 mmol). The mixture was stirred at 70° C. for 12 hr. On completion, the reaction mixture was filtered and concentrated under reduced pressure to give a residue. The crude product was purified by reversed-phase HPLC (0.1% NH₃.H₂O condition) to give the title compound (2 g, 83% yield) as a yellow solid. LC/MS (ESI, m/z): [M+1]+=237.2.

Step 2-5-Ethyl-3-(2-(methoxymethoxy)phenyl)-6,7,8,9-tetrahydro-5H-pyrido[3′,4′:4,5]pyrrolo[2,3-c]pyridazine. A mixture of [2-(methoxymethoxy)phenyl]boronic acid (3.08 g, 16.9 mmol, CAS #115377-93-0), 12-chloro-3-ethyl-4,8,10,11-tetrazatricyclo[7.4.0.02,7]trideca-1(9),2(7),10,12-tetraene (2 g, 8.45 mmol), BrettPhos Pd G3 (766 mg, 845 umol), and K₂CO₃ (5.84 g, 42.3 mmol) in dioxane (40 mL) and H₂O (20 mL) was degassed and purged with N₂ three times. Then the mixture was stirred at 80° C. for 12 hours under N₂ atmosphere. On completion, the reaction mixture was concentrated under reduced pressure to remove water. The residue was diluted with DCM (10 mL) and extracted with DCM (20 mL×3). The combined organic layers were dried over Na₂SO₄, filtered and concentrated under reduced pressure to give a residue. The residue was purified by column chromatography (SiO₂, Dichloromethane: Methanol=1:0 to 10:1) to give the title compound (1.2 g, 41% yield) as a yellow solid. LC-MS (ESI⁺) m/z 339.1(M+H)⁺.

(S)-tert-butyl 6-(4-(2-(5-ethyl-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate (Intermediate IW) and (R)-tert-butyl 6-(4-(2-(5-ethyl-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate (Intermediate IX)

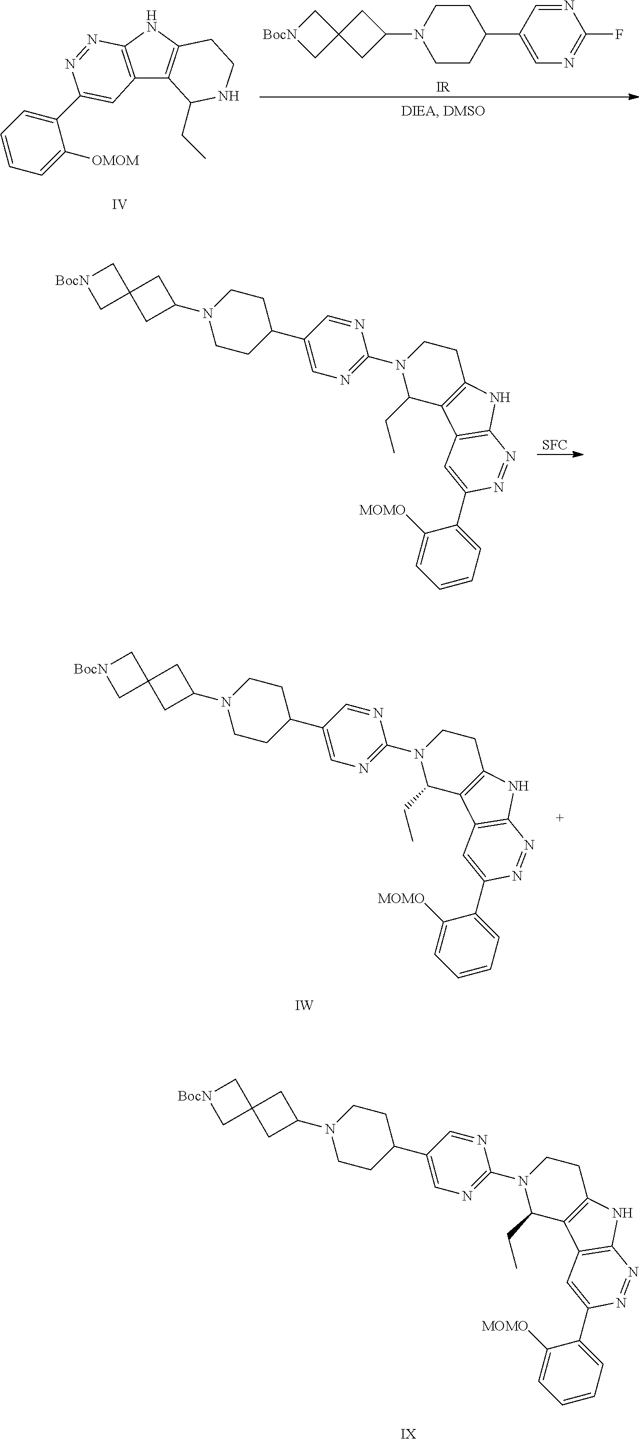

Step 1-Tert-butyl 6-(4-(2-(5-ethyl-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate. A mixture of 3-ethyl-12-[2-(methoxymethoxy)phenyl]-4,8,10,11-tetrazatricyclo[7.4.0.02,7]trideca-1(9),2(7),10,12-tetraene (1.2 g, 3.55 mmol, Intermediate IV), tert-butyl 6-[4-(2-fluoropyrimidin-5-yl)-1-piperidyl]-2-azaspiro[3.3]heptane-2-carboxylate (2.00 g, 5.32 mmol, Intermediate IR), DIEA (2.29 g, 17.7 mmol) in DMSO (5 mL) was degassed and purged with N₂ three times. Then the mixture was stirred at 80° C. for 12 hours under N₂ atmosphere. The reaction mixture was purified directly. The residue was purified by reversed phase flash chromatography (FA condition) to give the title compound (1.48 g, 57% yield, FA) as a white solid. LC-MS (ESI⁺) m/z 695.3(M+H)⁺.

Step 2-(S)-tert-butyl 6-(4-(2-(5-ethyl-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate and (R)-tert-butyl 6-(4-(2-(5-ethyl-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate. Tert-butyl 6-(4-(2-(5-ethyl-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate (1.48 g) was purified by SFC (column: DAICEL CHIRALCEL OD (250 mm×30 mm, 10 um); mobile phase: [0.1% NH₃H₂O MEOH]; B %: 45%-45%, 2.7; 90 min) to give tert-butyl 6-[4-[2-[(3S)-3-ethyl-12-[2-(methoxymethoxy)phenyl]-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carboxylate (680 mg, 979 umol, 45.95% yield. Retain time: 2.912 min) and, tert-butyl 6-[4-[2-[(3R)-3-ethyl-12-[2-(methoxymethoxy)phenyl]-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carboxylate (720 mg, 1.04 mmol, 48.65% yield, Retain time: 3.383 min) as a yellow solids. LC-MS (ESI⁺) m/z 695.3(M+H)⁺.

### 7-[1-(2,6-Dioxo-3-piperidyl)-3-methyl-2-oxo-benzimidazol-5-yl]hept-6-ynal (Intermediate IY)

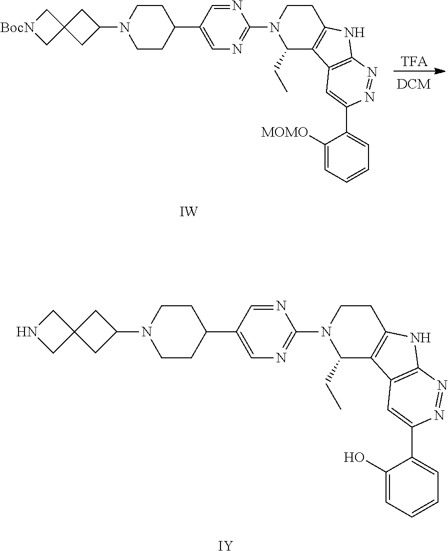

A mixture of tert-butyl 6-[4-[2-[(3S)-3-ethyl-12-[2-(methoxymethoxy)phenyl]-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carboxylate (200 mg, 300 umol, Intermediate IW), TFA (1.54 g, 13.5 mmol) in DCM (2 mL) was degassed and purged with N₂ three times. Then the mixture was stirred at 25° C. for 30 mins under N₂ atmosphere. The residue was purified by reversed phase Flash (FA condition) to give the title compound (100 mg, 62% yield, FA) as a white solid. LC-MS (ESI⁺) m/z 551.3 (M+H)⁺.

### Methyl 2-(2-(aminomethyl)-5-ethynylphenoxy)acetate (Intermediate IZ)

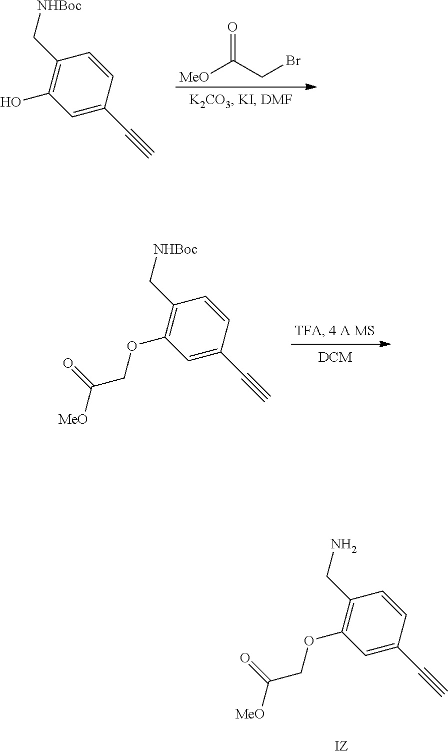 [SMILES: C#Cc1ccc(CNC(=O)OC(C)(C)C)c(O)c1 ; COC(=O)CBr ; C#Cc1ccc(CNC(=O)OC(C)(C)C)c(OCC(=O)OC)c1 ; C#Cc1ccc(CN)c(OCC(=O)OC)c1]

Step 1-Methyl 2-(2-(((tert-butoxycarbonyl)amino)methyl)-5-ethynylphenoxy)acetate. To a solution of tert-butyl N-[(4-ethynyl-2-hydroxy-phenyl)methyl]carbamate (1.3 g, 5.26 mmol, synthesized via Steps 1-3 of Intermediate HY) and methyl 2-bromoacetate (1.21 g, 7.89 mmol, 744.62 uL, CAS #96-32-2) in DMF (20 mL) was added K₂CO₃ (2.18 g, 15.77 mmol) and KI (174.53 mg, 1.05 mmol). The mixture was stirred at 75° C. for 2 hr. On completion, the reaction mixture was quenched with sat. NH₄Cl (10 mL) at 25° C., and then diluted with EA (10 mL) and extracted with EA (10 mL×3). The combined organic layers were washed with NaCl (10 mL×3), dried over Na₂SO₄, filtered and concentrated under reduced pressure to give a residue. The crude product was purified by column chromatography (SiO₂, Petroleum ether/Ethyl acetate=20/1 to 10/1) to give the title compound (1.28 g, 74% yield) as a white solid. ¹H NMR (400 MHz, DMSO-d6) δ ppm 1.40 (s, 9H) 3.70 (s, 3H) 4.13-4.18 (m, 2H) 4.88 (s, 2H) 7.00 (s, 1H) 7.07-7.15 (m, 2H) 7.20-7.23 (m, 1H). LC-MS (ESI+) m/z 342.2 (M+Na)⁺.

Step 2-Methyl 2-(2-(aminomethyl)-5-ethynylphenoxy)acetate. To a solution of methyl 2-[2-[(tert-butoxycarbonylamino)methyl]-5-ethynyl-phenoxy]acetate (1.2 g, 3.8 mmol) in DCM (12 mL) was added 4 Å molecular sieves (50 mg, 4 mmol) and TFA (1.80 g, 15.8 mmol, 1.17 mL). The mixture was then stirred at 25° C. for 1 hr. On completion, the reaction mixture was filtered and concentrated in vacuo to give the title compound (2 g) as a yellow oil. LC-MS (ESI+) m/z 203.1 (M−NH₂)⁺.

### Methyl 2-(2-(((2S,4R)-1-((S)-2-amino-3,3-dimethylbutanoyl)-4-hydroxypyrrolidine-2-carboxamido)methyl)-5-ethynylphenoxy)acetate (Intermediate JA)

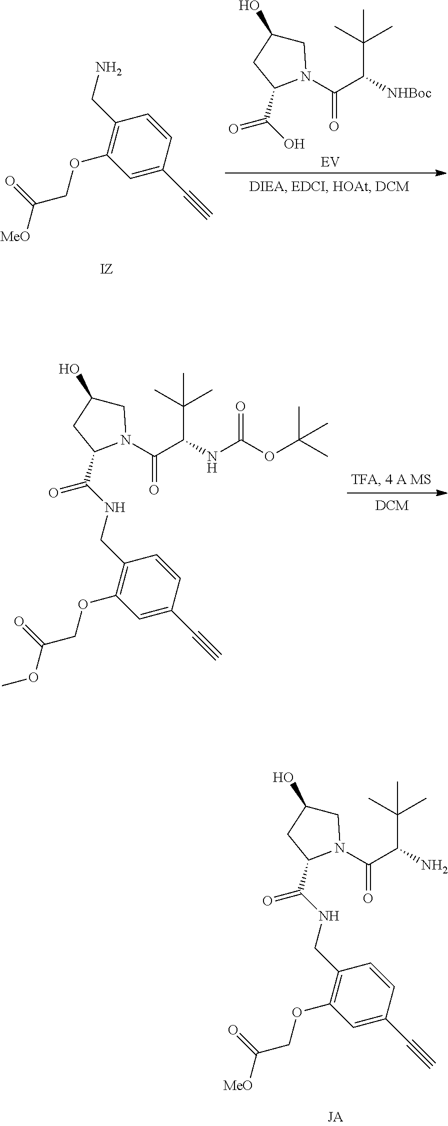 [SMILES: C#Cc1ccc(CN)c(OCC(=O)OC)c1 ; CC(C)(C)OC(=O)N[C@H](C(=O)N1C[C@H](O)C[C@H]1C(=O)O)C(C)(C)C ; C#Cc1ccc(CNC(=O)[C@@H]2C[C@@H](O)CN2C(=O)[C@@H](NC(=O)OC(C)(C)C)C(C)(C)C)c(OCC(=O)OC)c1 ; C#Cc1ccc(CNC(=O)[C@@H]2C[C@@H](O)CN2C(=O)[C@@H](N)C(C)(C)C)c(OCC(=O)OC)c1]

Step 1-Methyl 2-(2-(((2S,4R)-1-((S)-2-((tert-butoxycarbonyl)amino)-3,3-dimethylbutanoyl)-4-hydroxypyrrolidine-2-carboxamido)methyl)-5-ethynylphenoxy)acetate. To a solution of (2S,4R)-1-[(2S)-2-(tert-butoxycarbonylamino)-3,3-dimethyl-butanoyl]-4-hydroxy-pyrrolidine-2-carboxylic acid (1.86 g, 5.40 mmol, CAS #630421-46-4, Intermediate EV) in DCM (35 mL) was added DIEA (3.49 g, 27.0 mmol, 4.70 mL) and EDCI (1.24 g, 6.48 mmol) and HOAt (882.20 mg, 6.48 mmol). Then methyl 2-[2-(aminomethyl)-5-ethynyl-phenoxy]acetate (1.8 g, 5.40 mmol, TFA, Intermediate IZ) was added and the resulting mixture was stirred at 25° C. for 12 h. On completion, the reaction mixture was concentrated in vacuo to give a residue. The residue was purified by column chromatography (SiO₂, Petroleum ether/Ethyl acetate=1/1 to 1/5) to give the title compound (1.5 g, 48% yield) as a white solid. LC-MS (ESI⁺) m/z 546.5 (M+H)⁺.

Step 2-Methyl 2-(2-(((2S,4R)-1-((S)-2-amino-3,3-dimethylbutanoyl)-4-hydroxypyrrolidine-2-carboxamido)methyl)-5-ethynylphenoxy)acetate. To a solution of methyl 2-[2-[[[(2S,4R)-1-[(2S)-2-(tert-butoxycarbonylamino)-3,3-dimethyl-butanoyl]-4-hydroxy-pyrrolidine-2-carbonyl]amino]methyl]-5-ethynyl-phenoxy]acetate (1.3 g, 2.4 mmol) in DCM (13 mL) was added 4 Å molecular sieves (0.3 g) and TFA (2.62 g, 23 mmol, 1.7 mL). The mixture was stirred at 25° C. for 12 hr. On completion, the reaction mixture was filtered and concentrated in vacuo to give a residue. The crude product was purified by reversed-phase HPLC(0.1% NH₃•H₂O). Then, the eluent was evaporated to remove organic solvents. The residual aqueous solution was lyophilized to give the title compound as a pale yellow solid (1 g, 79% yield). LC-MS (ESI⁺) m/z 466.2 (M+H)⁺.

### Methyl 2-(2-(((2S,4R)-1-((S)-3,3-dimethyl-2-((phenoxycarbonyl)amino)butanoyl)-4-hydroxypyrrolidine-2-carboxamido)methyl)-5-ethynylphenoxy)acetate (Intermediate JB)

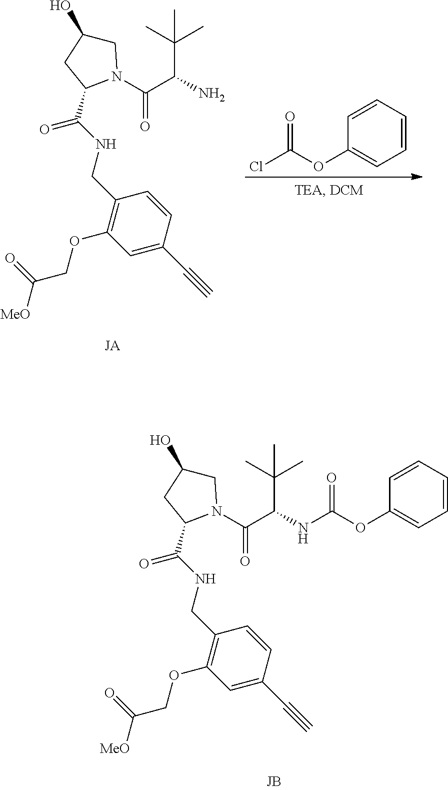 [SMILES: C#Cc1ccc(CNC(=O)[C@@H]2C[C@@H](O)CN2C(=O)[C@@H](N)C(C)(C)C)c(OCC(=O)OC)c1 ; O=C(Cl)Oc1ccccc1 ; C#Cc1ccc(CNC(=O)[C@@H]2C[C@@H](O)CN2C(=O)[C@@H](NC(=O)Oc2ccccc2)C(C)(C)C)c(OCC(=O)OC)c1]

To a solution of methyl 2-[2-[[[(2S,4R)-1-[(2S)-2-amino-3,3-dimethyl-butanoyl]-4-hydroxy-pyrrolidine-2-carbonyl]amino]methyl]-5-ethynyl-phenoxy]acetate (0.8 g, 1.80 mmol, Intermediate JA) in DCM (16 mL) was added TEA (545.12 mg, 5.39 mmol, 749.82 uL) and phenyl carbonochloridate (337.38 mg, 2.15 mmol, CAS #1885-14-9) at 0° C. The mixture was stirred at 0° C. for 0.25 hr. On completion, the reaction mixture was quenched with NH₄Cl (5 mL) at 25° C., and then diluted with DCM (5 mL) and extracted with DCM (10 mL×3). The combined organic layers were washed with sat. NaCL (10 mL×3), dried over Na₂SO₄, filtered and concentrated in vacuo to give a residue. The residue was purified by column chromatography (SiO₂, Petroleum ether/Ethyl acetate=1/1 to 1/5) to give the title compound (0.5 g, 44% yield) as a white solid. LC-MS (ESI⁺) m/z 566.4 (M+H)⁺.

### (S)-1-(4-ethynylphenyl)ethanamine (Intermediate JC)

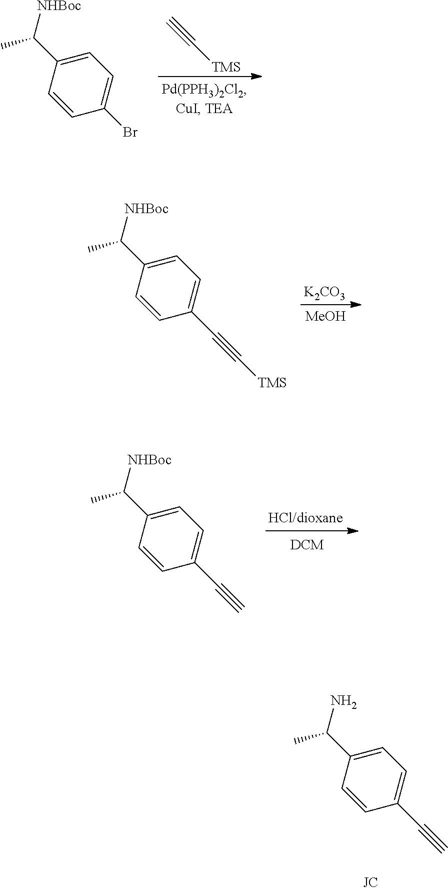

(S)-tert-butyl (1-(4-((trimethylsilyl)ethynyl)phenyl)ethyl)carbamate. A mixture of tert-butyl N-[(1S)-1-(4-bromophenyl)ethyl]carbamate (2 g, 7 mmol), ethynyl(trimethyl)silane (5.23 g, 53.3 mmol, 7.38 mL), Pd(PPh₃)₂Cl₂ (468 mg, 666 umol), and CuI (254 mg, 1.33 mmol) in TEA (20 mL) was degassed and purged with N₂ three times. Then the mixture was stirred at 80° C. for 24 hours under N₂ atmosphere. On completion, the reaction mixture was diluted with H₂O (50 mL) and extracted with EA (60 mL×3). The combined organic layers were dried over Na₂SO₄, filtered and concentrated under reduced pressure to give a residue. The residue was purified by column chromatography (SiO₂, Petroleum ether/Ethyl acetate=I/O to 10/1) to give the title compound (1.80 g, 72% yield) as a yellow oil. LC-MS (ESI⁺) m z 340.1 (M+H)⁺.

Step 2-(S)-tert-butyl (1-(4-ethynylphenyl)ethyl)carbamate. To a solution of tert-butyl N-[(1S)-1-[4-(2-trimethylsilylethynyl)phenyl]ethyl]carbamate (800 mg, 2.52 mmol) in MeOH (8 mL) was added K₂CO₃ (696 mg, 5.04 mmol). The mixture was stirred at 25° C. for 3.5 hr. On completion, the reaction mixture was diluted with H₂O (20 mL) and extracted with EA (25 mL×4). The combined organic layers were dried over Na₂SO₄, filtered and concentrated under reduced pressure to give the title compound (600 mg) as a yellow solid. LC-MS (ESI⁺) m/z 268.2 (M+Na)⁺.

Step 3-(S)-1-(4-ethynylphenyl)ethanamine. To a solution of tert-butyl N-[(1S)-1-(4-ethynylphenyl)ethyl]carbamate (600 mg, 2.45 mmol) in DCM (12 mL) was added HCl/dioxane (4 M, 3 mL). The mixture was then stirred at 25° C. for 1 hr. On completion, the reaction mixture was concentrated under reduced pressure to give the title compound as a yellow solid (492 mg). LC-MS (ESI⁺) m/z 340.1 (M−16)⁺.

### Tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate (Intermediate JD)

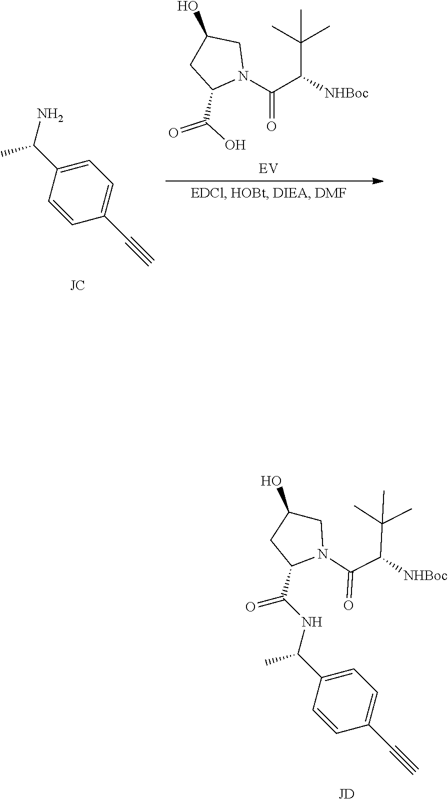 [SMILES: C#Cc1ccc([C@H](C)N)cc1 ; CC(C)(C)OC(=O)N[C@H](C(=O)N1C[C@H](O)C[C@H]1C(=O)O)C(C)(C)C ; C#Cc1ccc([C@H](C)NC(=O)[C@@H]2C[C@@H](O)CN2C(=O)[C@@H](NC(=O)OC(C)(C)C)C(C)(C)C)cc1]

To a solution of (2S,4R)-1-[(2S)-2-(tert-butoxycarbonylamino)-3,3-dimethyl-butanoyl]-4-hydroxy-pyrrolidine-2-carboxylic acid (938 mg, 2.72 mmol, CAS #630421-46-4, Intermediate EV) in DMF (10 mL) was added EDCI (712 mg, 3.72 mmol) and HOBt (502 mg, 3.72 mmol) and the mixture was stirred at 0° C. for 0.5 hr. Then (1S)-1-(4-ethynylphenyl)ethanamine (450 mg, 2.48 mmol, HCl, Intermediate JC) and DIEA (3.20 g, 24.8 mmol) was added and the resulting mixture was stirred at 25° C. for 1 hour. On completion, the reaction mixture was diluted with H₂O (40 mL) and extracted with EA (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=I/O to 1/1) to give the title compound (1.00 g, 80% yield) as a yellow solid. ¹H NMR (400 MHz, DMSO-d₆) δ=8.38 (d, J=7.6 Hz, 1H), 7.41 (d, J=8.3 Hz, 2H), 7.27 (d, J=8.3 Hz, 2H), 6.40 (d, J=9.1 Hz, 1H), 5.11 (d, J=3.3 Hz, 1H), 4.85 (quin, J=7.1 Hz, 1H), 4.42 (t, J=7.9 Hz, 1H), 4.27 (s, 1H), 4.17-4.07 (m, 2H), 3.62-3.51 (m, 2H), 2.00-1.96 (m, 1H), 1.73 (ddd, J=4.6, 8.2, 12.7 Hz, 1H), 1.38 (s, 9H), 1.32 (d, J=7.0 Hz, 3H), 0.92 (s, 9H). LC-MS (ESI⁺) m/z 472.2 (M+H)⁺.

### Phenyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate (Intermediate JE)

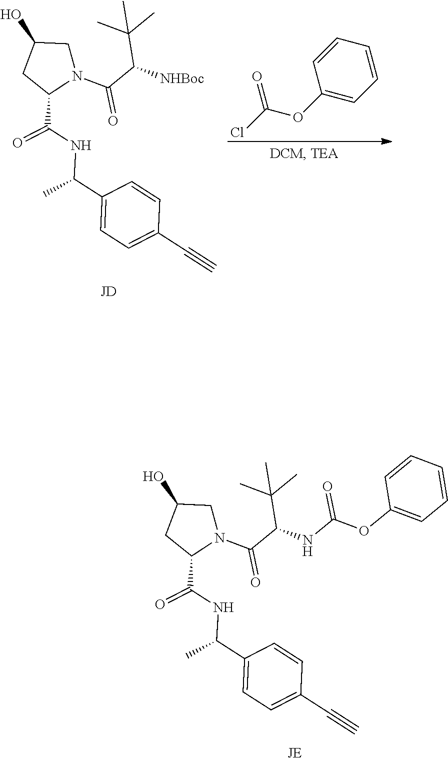 [SMILES: C#Cc1ccc([C@H](C)NC(=O)[C@@H]2C[C@@H](O)CN2C(=O)[C@@H](NC(=O)OC(C)(C)C)C(C)(C)C)cc1 ; O=C(Cl)Oc1ccccc1 ; C#Cc1ccc([C@H](C)NC(=O)[C@@H]2C[C@@H](O)CN2C(=O)[C@@H](NC(=O)Oc2ccccc2)C(C)(C)C)cc1]

To a solution of (2S,4R)-1-[(2S)-2-amino-3,3-dimethyl-butanoyl]-N-[(1S)-1-(4-ethynylphenyl)ethyl]-4-hydroxy-pyrrolidine-2-carboxamide (100 mg, 300 umol Intermediate JD) in DCM (10 mL) was added TEA (136 mg, 1.35 mmol, 187 uL) and phenyl carbonochloridate (63.2 mg, 404 umol, 50.6 uL CAS #1885-14-9). The mixture was stirred at 0° C. for 10 min. On completion, the reaction mixture was partitioned between DCM and brine. The organic phase was separated, washed with brine, dried with anhydrous sodium sulfate, filtered and concentrated under reduced pressure to give the title compound (132 mg) as a white solid. LC-MS (ESI⁺) m/z 492.1(M+H)⁺.

### (2S,4R)-1-[(2S)-3,3-dimethyl-2-[(2-oxospiro[3.5]nonane-7-carbonyl)amino]butanoyl]-N-[(4-ethynylphenyl)methyl]-4-hydroxy-pyrrolidine-2-carboxamide (Intermediate JF)

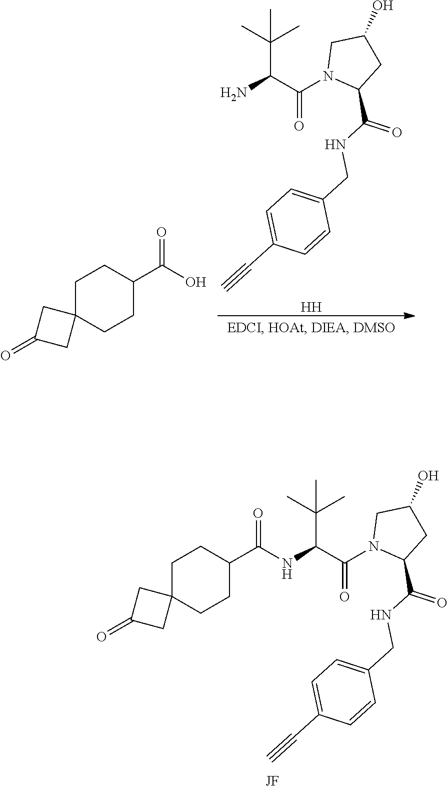

To a solution of 2-oxospiro[3.5]nonane-7-carboxylic acid (1 g, 5.49 mmol, CAS #2167815-01-0) and (2S,4R)-1-[(2S)-2-amino-3,3-dimethyl-butanoyl]-N-[(4-ethynylphenyl)methyl]-4-hydroxy-pyrrolidine-2-carboxamide (1.57 g, 4.39 mmol, Intermediate HH) in DMSO (10 mL) was added EDCI (1.58 g, 8.23 mmol) and HOAt (1.12 g, 8.23 mmol) and DIEA (4.26 g, 32.9 mmol). The mixture was stirred at 25° C. for 3 hours. On completion, the reaction mixture was partitioned between water (100 mL) and ethyl acetate (60 mL). The organic phase was separated, washed with brine (20 mL×3), dried over anhydrous sodium sulfate filtered and concentrated under reduced pressure to give a residue. The residue was purified by column chromatography (SiO₂, DCM/MeOH=20/1 to 10/1) to give title compound (1 g, 26% yield) as a yellow oil. LC-MS (ESI⁺) m/z 522.3 (M+H)⁺.

### (2S,4R)-1-((S)-3,3-dimethyl-2-(methylamino)butanoyl)-N-(4-ethynylbenzyl)-4-hydroxypyrrolidine-2-carboxamide (Intermediate JG)

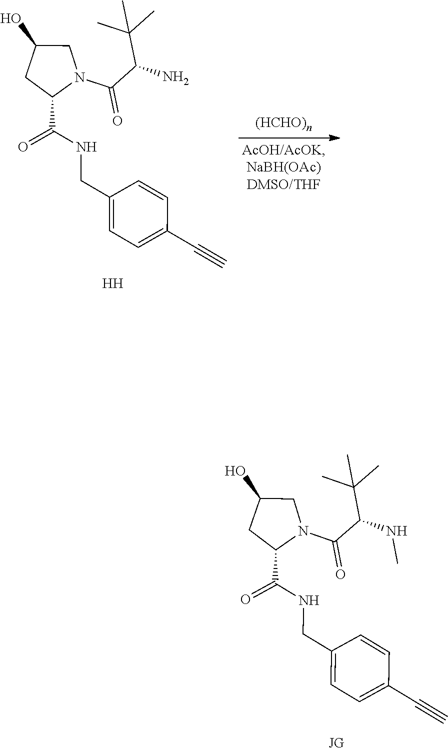

To a solution of (2S,4R)-1-[(2S)-2-amino-3,3-dimethyl-butanoyl]-N-[(4-ethynylphenyl)methyl]-4-hydroxy-pyrrolidine-2-carboxamide (100 mg, 300 umol, Intermediate HH) in THE (4 mL) and DMSO (4 mL) was added AcOK (82.4 mg, 839 umol), AcOH (50.40 mg, 839.29 umol, 48.00 uL), formaldehyde (25.2 mg, 839 umol, 23.1 uL) and stirred at 25° C. for 0.5 hour. Then NaBH(OAc)₃ (178 mg, 839 umol) was added at 0° C. and stirred at 25° C. for another 2 hours. On completion, the reaction was added water (2 ml), and then concentrated under reduced pressure to give the residue. The residue was purified by prep-HPLC (NH₃·H₂O) to give the title compound (100 mg, 25% yield) as a white solid. LC-MS (ESI⁺) m/z 372.0 (M+H)⁺.

### Phenyl ((S)-1-((2S,4R)-2-((4-ethynylbenzyl)carbamoyl)-4-hydroxypyrrolidin-1-yl)-3,3-dimethyl-1-oxobutan-2-yl)(methyl)carbamate (Intermediate JH)

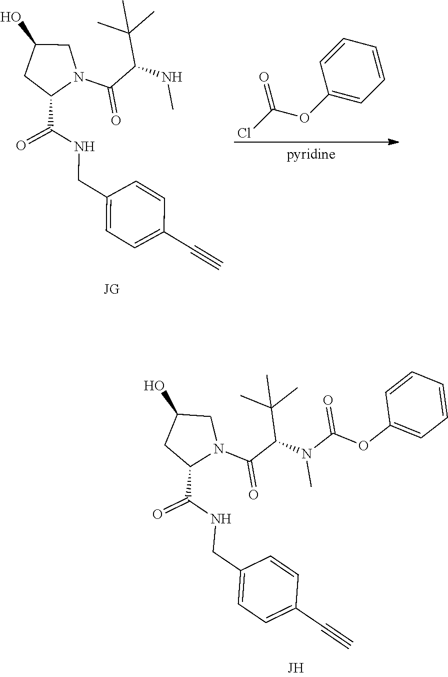 [SMILES: C#Cc1ccc(CNC(=O)[C@@H]2C[C@@H](O)CN2C(=O)[C@@H](NC)C(C)(C)C)cc1 ; O=C(Cl)Oc1ccccc1 ; C#Cc1ccc(CNC(=O)[C@@H]2C[C@@H](O)CN2C(=O)[C@@H](N(C)C(=O)Oc2ccccc2)C(C)(C)C)cc1]

To a solution of (2S,4R)-1-[(2S)-3,3-dimethyl-2-(methylamino)butanoyl]-N-[(4-ethynylphenyl)methyl]-4-hydroxy-pyrrolidine-2-carboxamide (50 mg, 135 umol, Intermediate JG) in pyridine (2 mL) was added phenyl carbonochloridate (21.1 mg, 135 umol, 16.9 uL). The mixture was stirred at 25° C. for 0.5 hour. On completion, the reaction mixture was added MeOH (3 mL), and then concentrated under reduced pressure to give a residue. The product was purified by pre-HPLC (FA) to give the title compound (50 mg, 63% yield) as a white solid. LC-MS (ESI⁺) m/z 492.2 (M+H)⁺.

### (3-(2-(methoxymethoxy)phenyl)-6,7,8,9-tetrahydro-5H-pyrido[3′,4′:4,5]pyrrolo[2,3-c]pyridazin-5-yl)methanol (Intermediate JI)

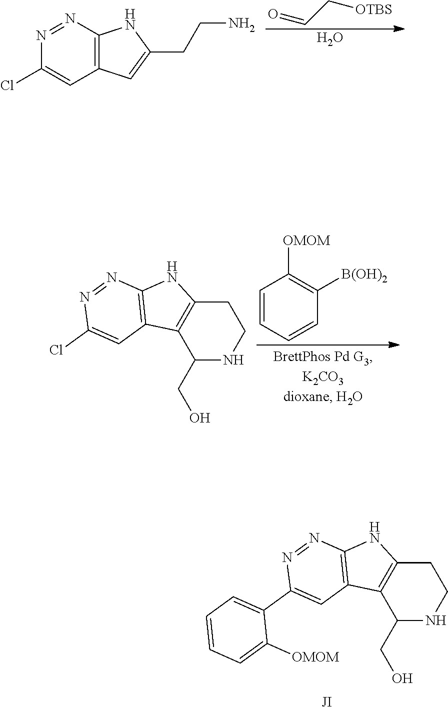

Step 1-3-chloro-5-ethyl-6,7,8,9-tetrahydro-5H-pyrido[3′,4′:4,5]pyrrolo[2,3-c]pyridazine. To a solution of 2-(3-chloro-7H-pyrrolo[2,3-c]pyridazin-6-yl)ethanamine (3.31 g, 16.8 mmol, synthesized via Steps 1-3 of Intermediate Y) in H₂O (30 mL) was added 2-[tert-butyl(dimethyl)silyl]oxyacetaldehyde (5.87 g, 33.7 mmol). The mixture was stirred at 70° C. for 12 hr. The reaction mixture was filtered and concentrated under reduced pressure to give a residue. The crude product was purified by reversed-phase HPLC (0.1% NH₃.H₂O condition) to give the title compound (3 g, 75% yield) as a yellow solid. LC/MS (ESI, m/z): [M+1]+=239.2.

Step 2-(3-(2-(methoxymethoxy)phenyl)-6,7,8,9-tetrahydro-5H-pyrido[3′,4′:4,5]pyrrolo[2,3-c]pyridazin-5-yl)methanol. A mixture of [2-(methoxymethoxy)phenyl]boronic acid (4.73 g, 25.98 mmol) (12-chloro-4,8,10,11-tetrazatricyclo[7.4.0.02,7]trideca-1(9),2(7),10,12-tetraen-3-yl)methanol (3.10 g, 12.99 mmol), BrettPhos Pd G3 (1.18 g, 1.30 mmol), and K₂CO₃ (8.98 g, 64.94 mmol) in dioxane (60 mL) and H₂O (30 mL) was degassed and purged with N₂ three times. Then the mixture was stirred at 80° C. for 12 hr under N₂ atmosphere. The reaction mixture was concentrated under reduced pressure to remove water. The residue was diluted with DCM (10 mL) and extracted with DCM (20 mL×3). The combined organic layers were dried over Na₂SO₄, filtered and concentrated under reduced pressure to give a residue. The residue was purified by column chromatography (SiO₂, Dichloromethane: Methanol=I/O to 10/1) to give the title compound (3.5 g, 78% yield) as a yellow solid. ¹H NMR (400 MHz, DMSO-d6) δ 12.23-11.89 (m, 1H), 8.03 (s, 1H), 7.69 (dd, J=1.6, 7.6 Hz, 1H), 7.42-7.34 (m, 1H), 7.24 (d, J=8.0 Hz, 1H), 7.14 (t, J=7.2 Hz, 1H), 5.23-5.16 (m, 2H), 4.80 (s, 1H), 4.18-3.98 (m, 2H), 3.79-3.71 (m, 1H), 3.64-3.56 (m, 1H), 3.26-3.13 (m, 4H), 2.96 (td, J=6.0, 12.2 Hz, 1H), 2.76 (s, 2H). LC-MS (ESI⁺) m z 341.2(M+H)⁺.

### (R)-tert-butyl 6-(4-(2-(5-(hydroxymethyl)-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate (Intermediate JJ) and (S)-tert-butyl 6-(4-(2-(5-(hydroxymethyl)-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate (Intermediate JK)

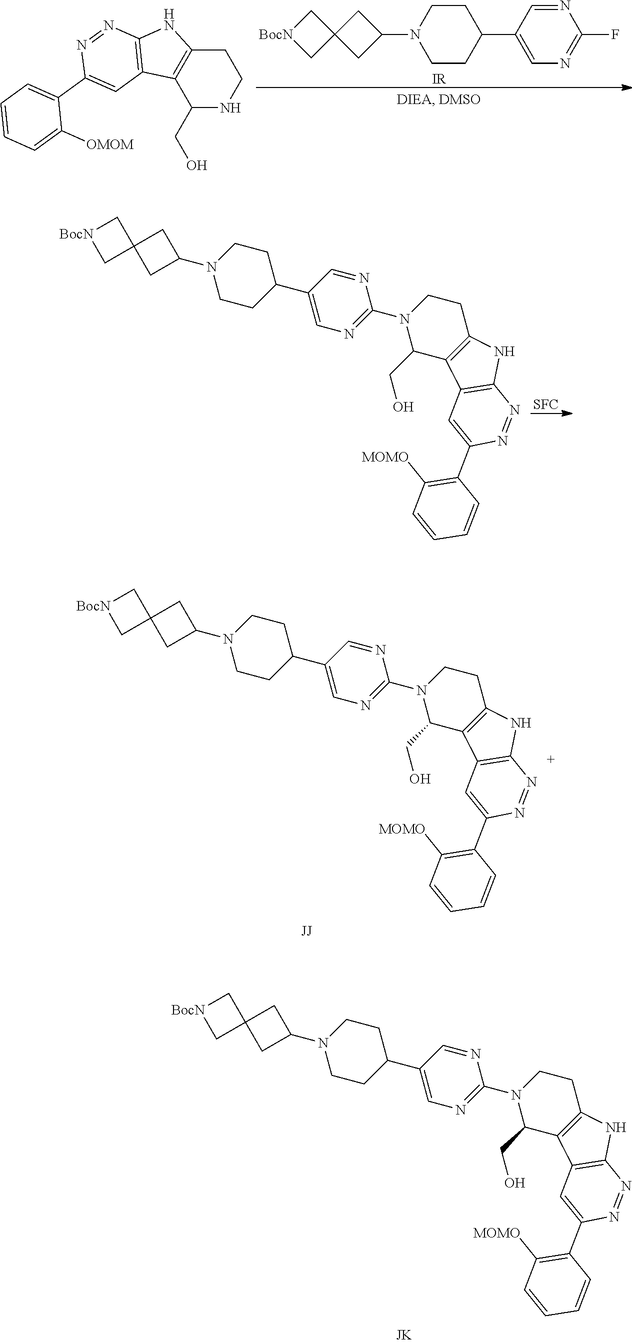

Step 1-Tert-butyl 6-(4-(2-(5-(hydroxymethyl)-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate. A mixture of (3-(2-(methoxymethoxy)phenyl)-6,7,8,9-tetrahydro-5H-pyrido[3′,4′:4,5]pyrrolo[2,3-c]pyridazin-5-yl)methanol (1.20 g, 3.55 mmol, Intermediate JI), tert-butyl 6-[4-(2-fluoropyrimidin-5-yl)-1-piperidyl]-2-azaspiro[3.3]heptane-2-carboxylate (2.00 g, 5.32 mmol, Intermediate IR), DIEA (2.29 g, 17.73 mmol) in DMSO (5 mL) was degassed and purged with N₂ three times. Then the mixture was stirred at 80° C. for 12 hr under N₂ atmosphere. On completion, the reaction mixture was purified directly by reversed phase flash chromatography (FA condition) to give the title compound (1.48 g, 57% yield) as a white solid. LC-MS (ESI⁺) m/z 697.3 (M+H)⁺.

Step 2-(R)-tert-butyl 6-(4-(2-(5-(hydroxymethyl)-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate and (S)-tert-butyl 6-(4-(2-(5-(hydroxymethyl)-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate. Tert-butyl 6-(4-(2-(5-(hydroxymethyl)-3-(2-(methoxymethoxy)phenyl)-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxylate (4.20 g) was purified by SFC (column: DAICEL CHIRALPAK IC (250 mm×30 mm, 10 um); mobile phase: [MeOH-ACN]; B %: 70%-70%, 4.4; 600 min) to give tert-butyl 6-[4-[2-[(3R)-3-(hydroxymethyl)-12-[2-(methoxymethoxy)phenyl]-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carboxylate (1.7 g, 41% yield), and tert-butyl 6-[4-[2-[(3S)-3-(hydroxymethyl)-12-[2-(methoxymethoxy)phenyl]-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carboxylate (2.2 g, 52% yield) were obtained as a yellow solids. LC-MS (ESI⁺) m/z 697.3 (M+H)⁺.

### (R)-2-(6-(5-(1-(2-azaspiro[3.3]heptan-6-yl)piperidin-4-yl)pyrimidin-2-yl)-5-(hydroxymethyl)-6,7,8,9-tetrahydro-5H-pyrido[3′,4′:4,5]pyrrolo[2,3-c]pyridazin-3-yl)phenol (Intermediate JL)

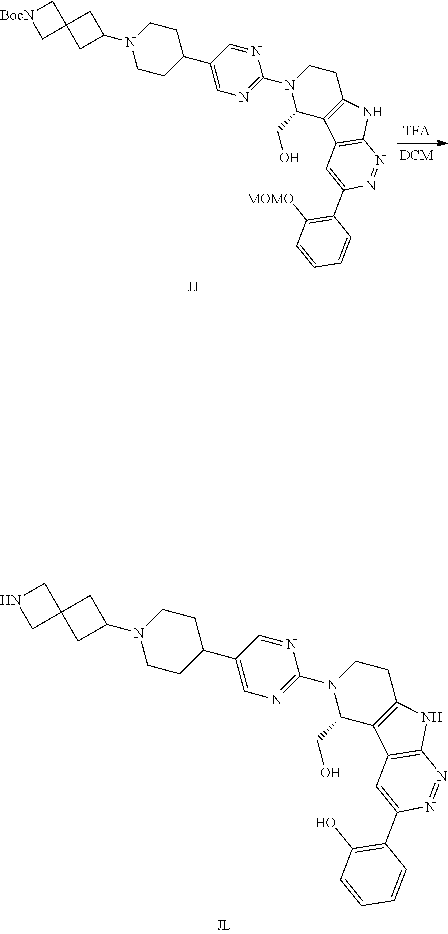

To a solution of tert-butyl 6-[4-[2-[(3R)-3-(hydroxymethyl)-12-[2-(methoxymethoxy)phenyl]-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carboxylate (50 mg, 72 umol, Intermediate JJ) in DCM (0.5 mL) was added TFA (245 mg, 2.15 mmol) and 4 Å molecular sieves (50 mg). The mixture was stirred at 25° C. for 12 hr. On completion, the reaction mixture was purified directly by reversed phase flash chromatography (FA condition) to give the title compound (15 mg, 29% yield, FA) as a white solid. LC-MS (ESI⁺) m/z 553.4 (M+H)⁺.

### (R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-4-yl)piperazine-1-carboxylate (Intermediate JM) and (S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-4-yl)piperazine-1-carboxylate (Intermediate JN)

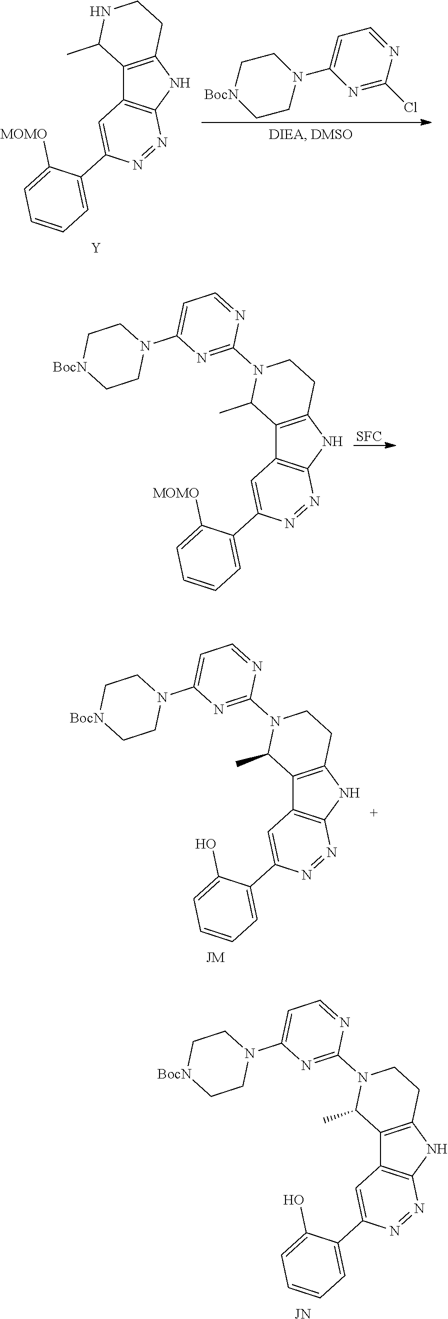

Step 1-Tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-4-yl)piperazine-1-carboxylate. To a solution of 12-[2-(methoxymethoxy)phenyl]-3-methyl-4,8,10,11-tetrazatricyclo[7.4.0.0^{2,7}]trideca-1(9),2(7),10,12-tetraene (3 g, 9.25 mmol, Intermediate Y) and tert-butyl 4-(2-chloropyrimidin-4-yl)piperazine-1-carboxylate (5.53 g, 18.5 mmol, CAS #221050-88-0) in DMSO (15 mL) was added DIEA (3.59 g, 27.8 mmol). The mixture was then stirred at 120° C. for 12 hours. Then the reaction mixture was partitioned between ethyl acetate (100 mL) and water (90 mL). The organic phase was separated, washed with brine (45 mL×2), dried over Na₂SO₄, filtered and concentrated under reduced pressure to give a residue. The residue was purified by column chromatography (SiO₂, DCM/MeOH=100/1 to 10/1) to give the title compound (2.8 g, 49% yield) as a yellow solid. LC-MS (ESI⁺) m/z 587.5 (M+H)⁺.

Step 2-(R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-4-yl)piperazine-1-carboxylate and (S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-4-yl)piperazine-1-carboxylate. Tert-butyl 4-[2-[12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-4-yl]piperazine-1-carboxylate (2.9 g, 4.94 mmol) was separated by SFC (column: DAICEL CHIRALCEL OD(250 mm×50 mm, 10 um); mobile phase: [0.1% NH₃H₂O MeOH]; B %: 45% −45%, 5.8; 260 min) to give tert-butyl 4-[2-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-4-yl]piperazine-1-carboxylate (1.1 g, 36% yield) as a yellow solid (LC-MS (ESI⁺) m/z 587.3 (M+H)⁺) and tert-butyl 4-[2-[(3S)-12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-4-yl]piperazine-1-carboxylate (1 g, 34% yield) as yellow solid (LC-MS (ESI⁺) m/z 587.3 (M+H)⁺).

### (R)-2-(5-methyl-6-(4-(piperazin-1-yl)pyrimidin-2-yl)-6,7,8,9-tetrahydro-5H-pyrido[3′,4′:4,5]pyrrolo[2,3-c]pyridazin-3-yl)phenol (Intermediate JO)

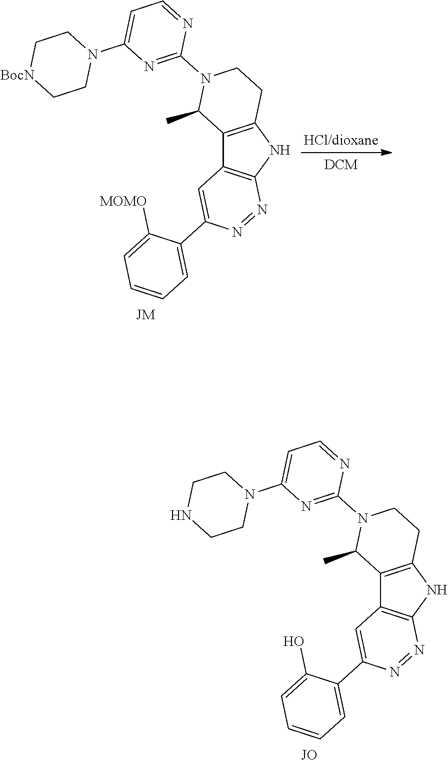

To a solution of tert-butyl 4-[2-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-4-yl]piperazine-1-carboxylate (100 mg, 170 umol, Intermediate JM) was added in DCM (1 mL) and HCl/dioxane (0.5 mL, 4 M). The mixture was then stirred at 25° C. for 12 hours. On completion, the reaction mixture was filtered and concentrated under reduced pressure to give a residue. The residue was purified by prep-HPLC (column: Welch Xtimate C18 150×25 mm×5 um; mobile phase: [wate r(0.05% HCl)-ACN]; B %: 0%-30%, 10 min) to give the title compound (33 mg, 41% yield) as a yellow solid. ¹H NMR (400 MHz, DMSO-d₆) δ=14.09 (s, 1H), 9.96 (s, 2H), 8.80 (s, 1H), 7.97 (d, J=7.6 Hz, 1H), 7.66 (d, J=7.2 Hz, 1H), 7.50-7.42 (m, 1H), 7.24 (d, J=8.1 Hz, 1H), 7.05 (t, J=7.6 Hz, 1H), 6.66 (d, J=7.2 Hz, 1H), 6.14 (s, 1H), 5.00-4.82 (m, 1H), 4.64-3.94 (m, 8H), 3.76 (s, 2H), 3.38 (s, 2H), 1.61 (d, J=6.4 Hz, 3H). LC-MS (ESI⁺) m z 443.0 (M+H)⁺.

### (S)-2-(2-hydroxyphenyl)-6a,7,9,10-tetrahydro-5H-pyrazino[1′,2′:4,5]pyrazino[2,3-c]pyridazin-8(6H)-yl)((R)-piperidin-3-yl)methanone (Intermediate JQ)

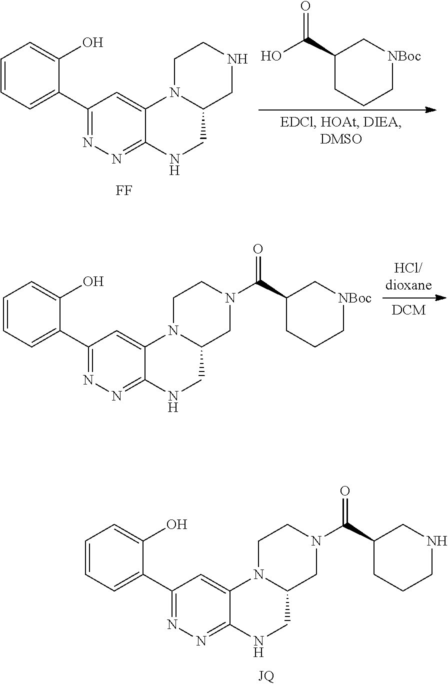

Step 1-(R)-tert-butyl 3-((S)-2-(2-hydroxyphenyl)-6,6a,7,8,9,10-hexahydro-5H-pyrazino[1′,2′:4,5]pyrazino[2,3-c]pyridazine-8-carbonyl)piperidine-1-carboxylate. To a solution of 2-[(1OR)-1,5,6,8,12-pentazatricyclo[8.4.0.0^{2,7}]tetradeca-2,4,6-trien-4-yl]phenol (547 mg, 1.71 mmol, Intermediate FF) and (3S)-1-tert-butoxycarbonylpiperidine-3-carboxylic acid (280 mg, 1.22 mmol, CAS #88495-54-9) in DMSO (4 mL) was added EDCI (351 mg, 1.83 mmol), HOAt (249 mg, 1.83 mmol) and DIEA (789 mg, 6.11 mmol). The mixture was then stirred at 25° C. for 12 hours. The reaction mixture was purified directly by reversed-phase HPLC (0.1% FA condition) to give the title compound (450 mg, 68% yield) as a yellow solid. LC-MS (ESI+) m/z 495.3 (M+H)⁺.

Step 2-[(10S)-4-(2-hydroxyphenyl)-1,5,6,8,12-pentazatricyclo[8.4.0.0^{2,7}]tetradeca-2,4,6-trien-12-yl]-[(3S)-3-piperidyl]methanone. To a solution of tert-butyl (3S)-3-[(10S)-4-(2-hydroxyphenyl)-1,5,6,8,12-pentazatricyclo[8.4.0.0^{2,7}]tetradeca-2,4,6-triene-12-carbonyl]piperidine-1-carboxylate (450 mg, 910 umol) was added in DCM (5 mL) and HCl/dioxane (1 mL). The mixture was then stirred at 25° C. for 1 hour. On completion, the reaction mixture was filtered and concentrated under reduced pressure to give the title compound (450 mg) as a pink solid. LC-MS (ESI+) m/z 395.2 (M+H)⁺.

### (R)-[1,4′-bipiperidin]-3-yl((S)-2-(2-hydroxyphenyl)-6a,7,9,10-tetrahydro-5H-pyrazino[1′,2′:4,5]pyrazino[2,3-c]pyridazin-8(6H)-yl)methanone (Intermediate JR)

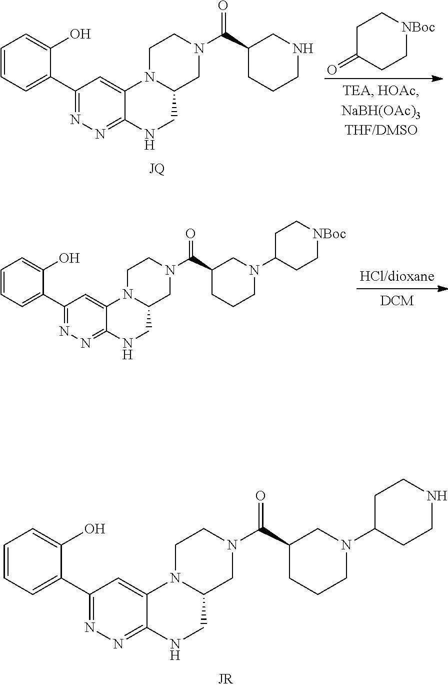

Step 1-(R)-tert-butyl 3-((S)-2-(2-hydroxyphenyl)-6,6a,7,8,9,10-hexahydro-5H-pyrazino[1′,2′:4,5]pyrazino[2,3-c]pyridazine-8-carbonyl)-[1,4′-bipiperidine]-1′-carboxylate. To a solution of [(10S)-4-(2-hydroxyphenyl)-1,5,6,8,12-pentazatricyclo[8.4.0.0^{2,7}]tetradeca-2,4,6-trien-12-yl]-[(3S)-3-piperidyl]methanone (380 mg, 882 umol, Intermediate JQ) in THF (4 mL) and DMSO (0.4 mL) was added TEA (178 mg, 1.76 mmol), HOAc (159 mg, 2.65 mmol) and tert-butyl 4-oxopiperidine-1-carboxylate (264 mg, 1.32 mmol, CAS #79099-07-3) and the reaction was stirred at 25° C. for 0.5 hour. Then NaBH₃CN (166 mg, 2.65 mmol) was added to the mixture and the mixture was stirred at 0-25° C. for 3 hours. On completion, the reaction mixture was filtered and concentrated under reduced pressure to give a residue. The crude product was purified by reversed-phase HPLC (0.1% FA condition) to give the title compound (90 mg, 17% yield) was as a colorless oil. LC-MS (ESI+) m/z 578.5 (M+H)⁺.

Step 2-(R)-[1,4′-bipiperidin]-3-yl((S)-2-(2-hydroxyphenyl)-6a,7,9,10-tetrahydro-5H-pyrazino[1′,2′:4,5]pyrazino[2,3-c]pyridazin-8(6H)-yl)methanone. To a solution of tert-butyl 4-[(3S)-3-[(10S)-4-(2-hydroxyphenyl)-1,5,6,8,12-pentazatricyclo[8.4.0.0^{2,7}]tetradeca-2,4,6-triene-12-carbonyl]-1-piperidyl]piperidine-1-carboxylate (100 mg, 173 umol) was added in DCM (0.5 mL) and HCl/dioxane (0.1 mL). The mixture was stirred at 25° C. for 1 hour. On completion, the reaction mixture was filtered and concentrated under reduced pressure to give the title compound (90 mg) as a yellow solid. LC-MS (ESI+) m/z 478.4 (M+H)⁺.

### (2S,4R)-4-((tert-butyldimethylsilyl)oxy)-N-(4-ethynylbenzyl)-1-((S)-2-hydroxy-3,3-dimethylbutanoyl)pyrrolidine-2-carboxamide (Intermediate JS)

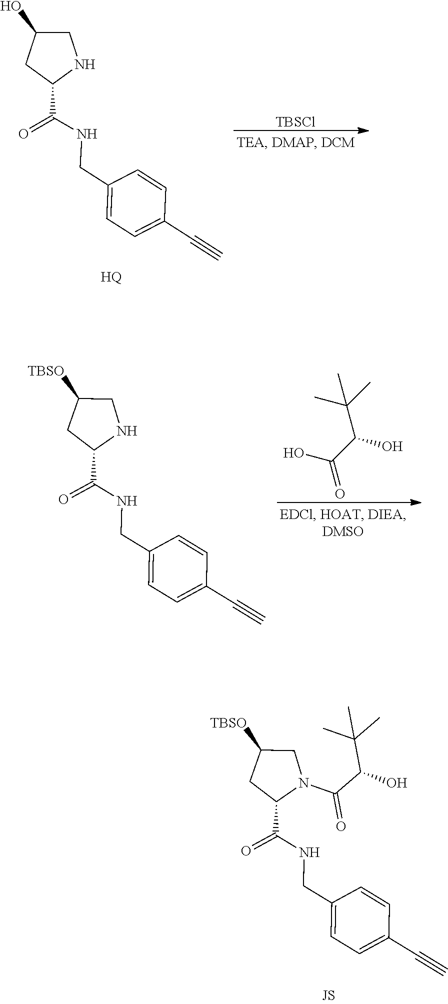

Step 1-(2S,4R)-4-((tert-butyldimethylsilyl)oxy)-N-(4-ethynylbenzyl)pyrrolidine-2-carboxamide. To a solution of (2S,4R)—N-[(4-ethynylphenyl)methyl]-4-hydroxy-pyrrolidine-2-carboxamide (900 mg, 4 mmol, Intermediate HQ) in DCM (10 mL) was added TEA (1.12 g, 11.0 mmol) and DMAP (180 mg, 1.47 mmol). Then TBSCl (1.11 g, 7.37 mmol) was added at 0° C. The mixture was stirred at 25° C. for 12 hours. On completion, the reaction mixture was diluted with H₂O (5 mL) and extracted with DCM (100 mL×3). The combined organic layers were washed with brine (30 mL×2), dried over Na₂SO₄, filtered and concentrated under reduced pressure to give the title compound (1.4 g) as a white solid. LC-MS (ESI⁺) m/z 359.3 (M+H)⁺.

Step 2-(2S,4R)-4-((tert-butyldimethylsilyl)oxy)-N-(4-ethynylbenzyl)-1-((S)-2-hydroxy-3,3-dimethylbutanoyl)pyrrolidine-2-carboxamide. To a solution of(2S,4R)-4-[tert-butyl(dimethyl)silyl]oxy-N-[(4-ethynylphenyl)methyl]pyrrolidine-2-carboxamide (500 mg, 1.4 mmol), (2S)-2-hydroxy-3,3-dimethyl-butanoic acid (202 mg, 1.53 mmol) in DMSO (5 mL) was added DIEA (540 mg, 4.18 mmol), EDCI (668 mg, 3.49 mmol) and HOAt (474 mg, 3.49 mmol). The mixture was then stirred at 25° C. for 5 hours. On completion, the reaction mixture was diluted with H₂O 10 mL and extracted with EA (20 mL×3). The combined organic layers were washed with brine (30 mL×2), dried over Na₂SO₄, filtered and concentrated under reduced pressure to give a residue. The residue was purified by column chromatography (SiO₂, Petroleum ether/Ethyl acetate=10/1 to 5/1) to give the title compound (520 mg, 73% yield) as a colorless oil. LC-MS (ESI⁺) m/z 473.4 (M+H)⁺.

### (S)-1-((2S,4R)-4-((tert-butyldimethylsilyl)oxy)-2-((4-ethynylbenzyl)carbamoyl)pyrrolidin-1-yl)-3,3-dimethyl-1-oxobutan-2-yl phenyl carbonate (Intermediate JT)

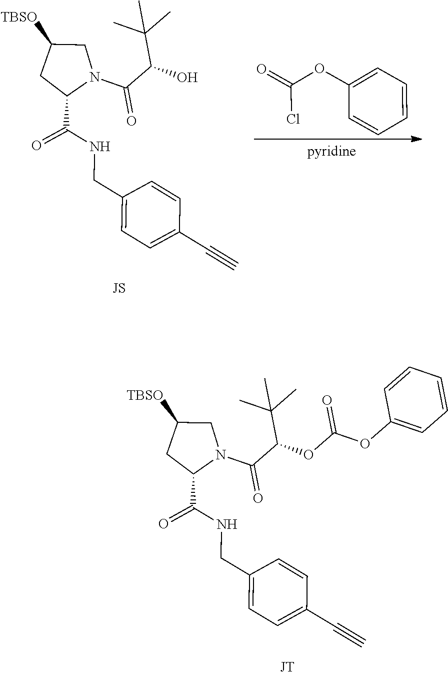 [SMILES: C#Cc1ccc(CNC(=O)[C@@H]2C[C@@H](O[Si](C)(C)C(C)(C)C)CN2C(=O)[C@@H](O)C(C)(C)C)cc1 ; O=C(Cl)Oc1ccccc1 ; C#Cc1ccc(CNC(=O)[C@@H]2C[C@@H](O[Si](C)(C)C(C)(C)C)CN2C(=O)[C@@H](OC(=O)Oc2ccccc2)C(C)(C)C)cc1]

To a solution of (2S,4R)-4-[tert-butyl(dimethyl)silyl]oxy-N-[(4-ethynylphenyl)methyl]-1-[(2S)-2-hydroxy-3,3-dimethyl-butanoyl]pyrrolidine-2-carboxamide (200 mg, 423 umol, Intermediate JS) in pyridine (2 mL) was added phenyl carbonochloridate (132 mg, 846 umol) at 0° C. The mixture was stirred at 25° C. for 3 hrs. On completion, the reaction mixture was quenched with MeOH (1 mL) at 0° C., and then filtered and concentrated under reduced pressure to give a residue. The residue was purified by prep-HPLC (FA condition) to give the title compound (180 mg, 64% yield, FA) as a white solid. LC-MS (ESI⁺) m/z 593.4 (M+H)⁺.

### (2S,4R)—N-(4-ethynylbenzyl)-4-hydroxy-1-((S)-2-((2-methoxyethyl)amino)-3,3-dimethylbutanoyl)pyrrolidine-2-carboxamide (Intermediate JU)

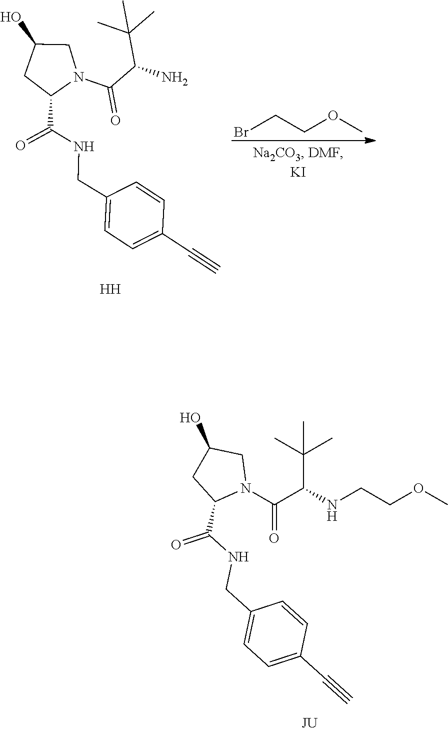

To a solution of (2S,4R)-1-[(2S)-2-amino-3,3-dimethyl-butanoyl]-N-[(4-ethynylphenyl)methyl]-4-hydroxy-pyrrolidine-2-carboxamide (0.5 g, 1.40 mmol, Intermediate HH) in DMF (10 mL) was added Na₂CO₃ (178 mg, 1.68 mmol), KI (232 mg, 1.40 mmol), and 1-bromo-2-methoxy-ethane (292 mg, 2.10 mmol, 197 uL). The mixture was stirred at 100° C. for 12 hrs. On completion, the reaction mixture was diluted with water (1 ml). The product was purified by pre-HPLC (NH₃·H₂O) to give the title compound (357 mg, 57% yield) as a white solid. LC-MS (ESI⁺) m/z 416.3 (M+H)⁺.

### Phenyl ((S)-1-((2S,4R)-2-((4-ethynylbenzyl)carbamoyl)-4-hydroxypyrrolidin-1-yl)-3,3-dimethyl-1-oxobutan-2-yl)(2-methoxyethyl)carbamate (Intermediate JV)

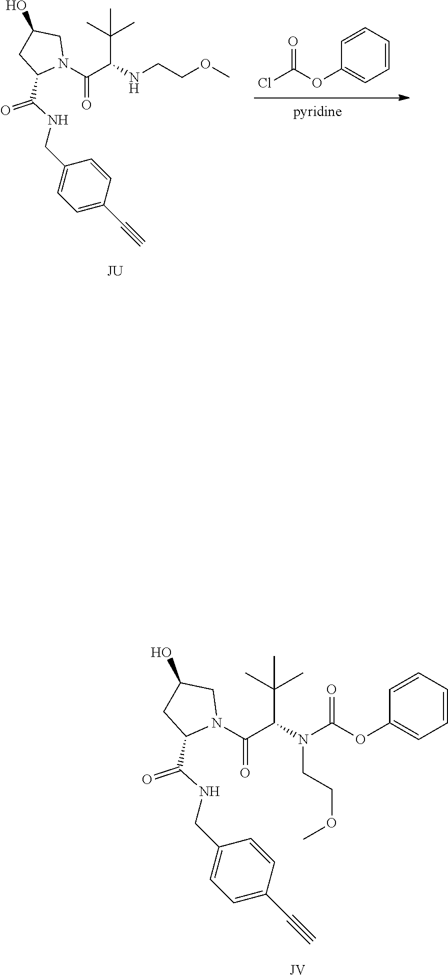

To a solution of (2S,4R)—N-[(4-ethynylphenyl)methyl]-4-hydroxy-1-[(2S)-2-(2-methoxyethylamino)-3,3-dimethyl-butanoyl]pyrrolidine-2-carboxamide (200 mg, 481 umol, Intermediate JU) in pyridine (5.88 g, 74.3 mmol, 6 mL) and phenyl carbonochloridate (90.4 mg, 578 umol, 72.3 uL). The mixture was then stirred at 25° C. for 1 hour. On completion, the reaction mixture was diluted with DMSO (1 mL). The residue was purified by prep-HPLC (FA) to give the title compound (150 mg, 47% yield) as a yellow oil. LC-MS (ESI⁺) m/z 536.2 (M+H)⁺.

### ((S)-2-(2-hydroxyphenyl)-6a,7,9,10-tetrahydro-5H-pyrazino[1′,2′:4,5]pyrazino[2,3-c]pyridazin-8(6H)-yl)((S)-piperidin-3-yl)methanone (Intermediate JW)

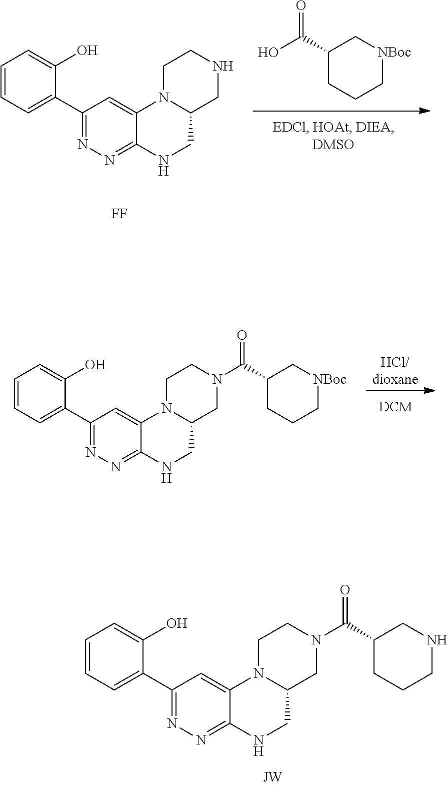 [SMILES: Oc1ccccc1-c1cc2c(nn1)NC[C@H]1CNCCN21 ; CC(C)(C)OC(=O)N1CCC[C@H](C(=O)O)C1 ; "FF" ; CC(C)(C)OC(=O)N1CCC[C@H](C(=O)N2CCN3c4cc(-c5ccccc5O)nnc4NC[C@H]3C2)C1 ; O=C([C@H]1CCCNC1)N1CCN2c3cc(-c4ccccc4O)nnc3NC[C@H]2C1]

Step 1-(S)-tert-butyl 3-((S)-2-(2-hydroxyphenyl)-6,6a,7,8,9,10-hexahydro-5H-pyrazino[1′,2′:4,5]pyrazino[2,3-c]pyridazine-8-carbonyl)piperidine-1-carboxylate. To a solution of 2-[(1OR)-1,5,6,8,12-pentazatricyclo[8.4.0.0^{2,7}]tetradeca-2,4,6-trien-4-yl]phenol (550 mg, 1.72 mmol, Intermediate FF) and (3S)-1-tert-butoxycarbonylpiperidine-3-carboxylic acid (394 mg, 1.72 mmol, CAS #88495-54-9) in DMSO (5 mL) was added EDCI (494 mg, 2.58 mmol) and HOAt (351 mg, 2.58 mmol) and DIEA (1.11 g, 8.60 mmol). The mixture was then stirred at 25° C. for 12 hrs. The reaction mixture was purified directly by reversed-phase HPLC (0.1% FA condition) to give the title compound (596 mg, 61% yield, FA) as a yellow solid. LC-MS (ESI⁺) m/z 495.3(M+H)⁺.

Step 2-((S)-2-(2-hydroxyphenyl)-6a,7,9,10-tetrahydro-5H-pyrazino[1′,2′:4,5]pyrazino[2,3-c]pyridazin-8(6H)-yl)((S)-piperidin-3-yl)methanone. To a solution of tert-butyl (3S)-3-[(10S)-4-(2-hydroxyphenyl)-1,5,6,8,12-pentazatricyclo[8.4.0.0^{2,7}]tetradeca-2,4,6-triene-12-carbonyl]piperidine-1-carboxylate (596 mg, 1.21 mmol) in DCM (6 mL) was added HCl/dioxane (1.2 mL). The mixture was then stirred at 25° C. for 1 hr. On completion, the reaction mixture was filtered and concentrated under reduced pressure to give the title compound (522 mg) as a yellow solid. LC-MS (ESI⁺) m/z 395.2(M+H)⁺.

### (S)-[1,4′-bipiperidin]-3-yl((S)-2-(2-hydroxyphenyl)-6a,7,9,10-tetrahydro-5H-pyrazino[1′,2′:4,5]pyrazino[2,3-c]pyridazin-8(6H)-yl)methanone (Intermediate JX)

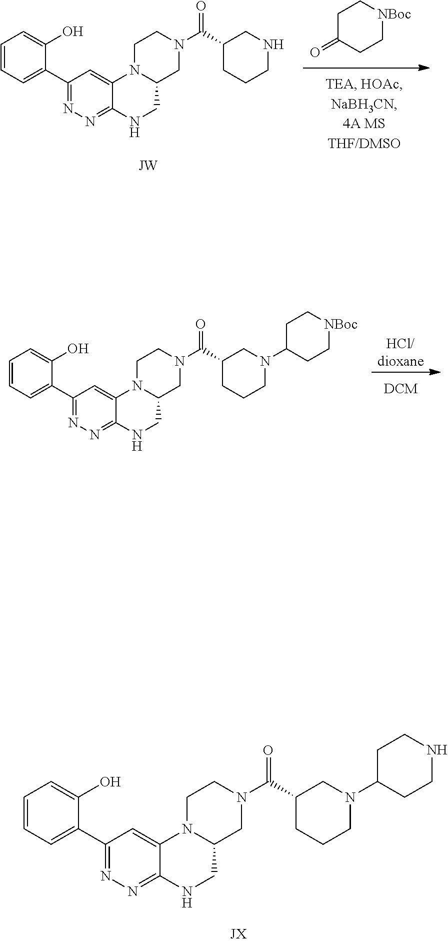 [SMILES: O=C([C@H]1CCCNC1)N1CCN2c3cc(-c4ccccc4O)nnc3NC[C@H]2C1 ; CC(C)(C)OC(=O)N1CCC(=O)CC1 ; "JW" ; CC(C)(C)OC(=O)N1CCC(N2CCC[C@H](C(=O)N3CCN4c5cc(-c6ccccc6O)nnc5NC[C@H]4C3)C2)CC1 ; O=C([C@H]1CCCN(C2CCNCC2)C1)N1CCN2c3cc(-c4ccccc4O)nnc3NC[C@H]2C1]

Step 1-(S)-tert-butyl 3-((S)-2-(2-hydroxyphenyl)-6,6a,7,8,9,10-hexahydro-5H-pyrazino[1′,2′:4,5]pyrazino[2,3-c]pyridazine-8-carbonyl)-[1,4′-bipiperidine]-1′-carboxylate. To a solution of [(10S)-4-(2-hydroxyphenyl)-1,5,6,8,12-pentazatricyclo[8.4.0.0^{2,7}]tetradeca-2,4,6-trien-12-yl]-[(3S)-3-piperidyl]methanone (75 mg, 174 umol, Intermediate JW) in THF (2 mL) and DMSO (0.2 mL) was added TEA (35.2 mg, 348 umol) and the mixture was stirred at 25° C. for 0.5 hour. Then tert-butyl 4-oxopiperidine-1-carboxylate (52 mg, 261 umol), HOAc (31.3 mg, 522 umol) and 4 Å molecular sieves (75 mg, 174.04 umol) were added and the reaction mixture was stirred at 25° C. for 0.5 hour. Finally, NaBH₃CN (32.8 mg, 522 umol) was added and the mixture was stirred at 0-25° C. for 3 hr. The reaction mixture was filtered and concentrated under reduced pressure to give a residue. The residue was purified by reversed-phase HPLC (0.1% FA condition) to give the title compound (72 mg, 64% yield, FA) as a yellow oil. LC-MS (ESI⁺) m/z 578.5 (M+H)⁺.

Step 2-(S)-[1,4′-bipiperidin]-3-yl((S)-2-(2-hydroxyphenyl)-6a,7,9,10-tetrahydro-5H-pyrazino[1′,2′:4,5]pyrazino[2,3-c]pyridazin-8(6H)-yl)methanone. To a solution of tert-butyl 4-[(3S)-3-[(10S)-4-(2-hydroxyphenyl)-1,5,6,8,12-pentazatricyclo[8.4.0.0^{2,7}]tetradeca-2,4,6-triene-12-carbonyl]-1-piperidyl]piperidine-1-carboxylate (91 mg, 157 umol) in DCM (1 mL) and HCl/dioxane (0.2 mL). The mixture was then stirred at 25° C. for 1 hour. On completion, the reaction mixture was filtered and concentrated under reduced pressure to give the title compound (82 mg) as a yellow solid. LC-MS (ESI⁺) m/z 478.4 (M+H)⁺.

### (2S,4R)-1-((S)-2-((2-(dimethylamino)ethyl)amino)-3,3-dimethylbutanoyl)-N-(4-ethynylbenzyl)-4-hydroxypyrrolidine-2-carboxamide (Intermediate JY)

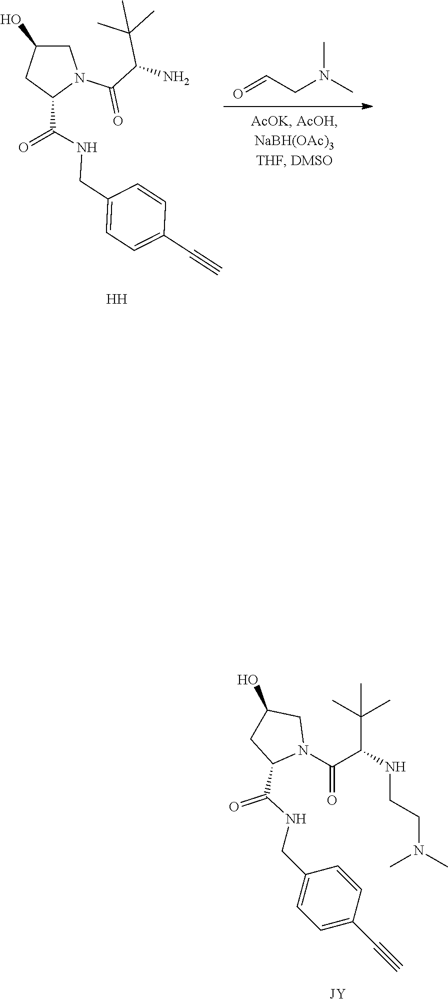

To a solution of (2S,4R)-1-((S)-2-amino-3,3-dimethylbutanoyl)-N-(4-ethynylbenzyl)-4-hydroxypyrrolidine-2-carboxamide (500 mg, 1.40 mmol, Intermediate HH) in THF (0.5 mL) and DMSO (0.5 mL) was added AcOK (411.85 mg, 4.20 mmol), AcOH (252.00 mg, 4.20 mmol) and 2-(dimethylamino)acetaldehyde (365.59 mg, 4.20 mmol) at 25° C. and stirred for 2 hrs. Next, NaBH₃CN (264 mg, 4.20 mmol) was added at 0° C. The mixture was then stirred at 25° C. for 10 hrs. On completion, the reaction mixture was concentrated in vacuo to give a residue. The crude product was purified by reversed-phase (0.1% NH3•H2O condition) to give the title compound (100 mg, 14% yield) as a yellow solid. LC-MS (ESI⁺) m/z 429.2 (M+H)⁺.

### Phenyl (2-(dimethylamino)ethyl)((S)-1-((2S,4R)-2-((4-ethynylbenzyl)carbamoyl)-4-hydroxypyrrolidin-1-yl)-3,3-dimethyl-1-oxobutan-2-yl)carbamate (Intermediate JZ)

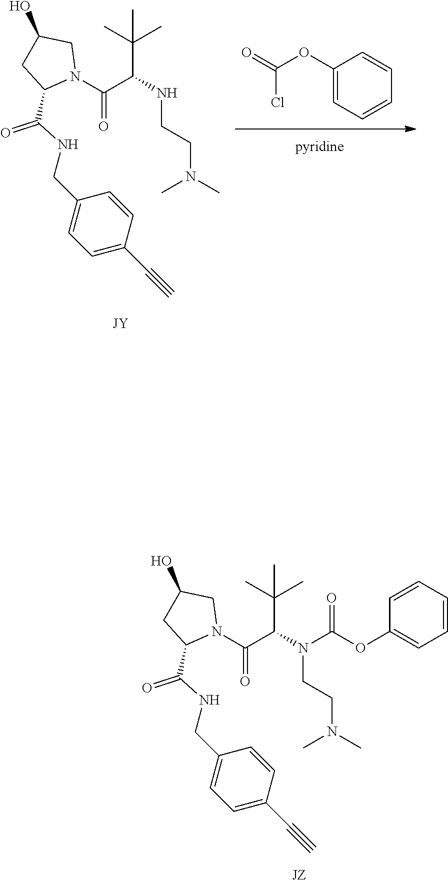 [SMILES: C#Cc1ccc(CNC(=O)[C@@H]2C[C@@H](O)CN2C(=O)[C@@H](NCCN(C)C)C(C)(C)C)cc1 ; O=C(Cl)Oc1ccccc1 ; "JY" ; C#Cc1ccc(CNC(=O)[C@@H]2C[C@@H](O)CN2C(=O)[C@@H](N(CCN(C)C)C(=O)Oc2ccccc2)C(C)(C)C)cc1]

To a mixture of (2S,4R)-1-((S)-2-((2-(dimethylamino)ethyl)amino)-3,3-dimethylbutanoyl)-N-(4-ethynylbenzyl)-4-hydroxypyrrolidine-2-carboxamide (80 mg, 200 umol, Intermediate JY) in pyridine (2.4 mL) was added phenyl carbonochloridate (35.07 mg, 224.0 umol). The mixture was then stirred at 25° C. for 1 hr. On completion, the reaction mixture was concentrated in vacuo to give a residue. The crude product was purified by reversed-phase (0.1% FA condition) to give the title compound (30 mg, 17% yield) as a brown solid. LC-MS (ESI⁺) m/z 549.5 (M+H)⁺.

### (2S,4R)-benzyl 1-((S)-2-amino-3,3-dimethylbutanoyl)-4-hydroxypyrrolidine-2-carboxylate (Intermediate KA)

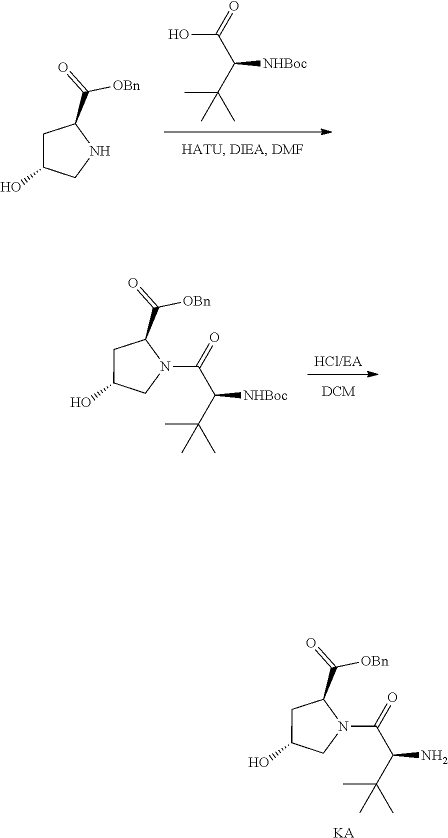

Step 1-(2S,4R)-benzyl 1-((S)-2-((tert-butoxycarbonyl)amino)-3,3-dimethylbutanoyl)-4-hydroxypyrrolidine-2-carboxylate. To a solution of (2S)-2-(tert-butoxycarbonylamino)-3,3-dimethyl-butanoic acid (8.97 g, 38.80 mmol, CAS #62965-35-9) in DMF (200 mL) was added HATU (17.70 g, 46.56 mmol) and DIEA (25.08 g, 194.0 mmol), then benzyl (2S,4R)-4-hydroxypyrrolidine-2-carboxylate (10 g, 40 mmol, HCl) was added. Then the mixture was stirred at 25° C. for 12 hr. On completion, the reaction mixture was quenched with NH₄Cl (200 mL) at 25° C., and then diluted with EA (200 mL) and extracted with EA (100 mL×3). The combined organic layers were washed with NaCl (100 mL×3), dried over Na₂SO₄, filtered and concentrated in vacuo to give a residue. The residue was purified by column chromatography (SiO₂, Petroleum ether/Ethyl acetate/DCM=10/1/1 to 1/1/0.1) to give the title compound (11 g, 63% yield) as a yellow solid. ¹H NMR (400 MHz, CHLOROFORM-d) δ ppm 0.93 (s, 9H) 1.34 (s, 9H) 1.94-1.87 (m, 1H) 1.98 (s, 1H) 2.18-2.01 (m, 2H) 2.29 (m, 1H) 2.82 (s, 3H) 2.89 (s, 3H) 3.62 (m, 1H) 4.16-3.92 (m, 2H) 4.42 (s, 1H) 4.68 (t, J=8.57 Hz, 1H) 5.24-5.02 (m, 3H) 7.20 (s, 1H) 7.33-7.22 (m, 4H) 7.95 (s, 1H). LC-MS (ESI⁺) m/z 435.1 (M+H)⁺.

Step 2-(2S,4R)-benzyl 1-((S)-2-amino-3,3-dimethylbutanoyl)-4-hydroxypyrrolidine-2-carboxylate. To a solution of benzyl (2S,4R)-1-[(2S)-2-(tert-butoxycarbonylamino)-3,3-dimethyl-butanoyl]-4-hydroxy-pyrrolidine-2-carboxylate (10 g, 20 mmol) in DCM (100 mL) was added HCl/EtOAc (4 M, 50 mL). The mixture was stirred at 25° C. for 0.5 hr. On completion, the reaction mixture was concentrated in vacuo to give the title compound (8.5 g, HCl) as a white solid. LC-MS (ESI⁺) m/z 335.0 (M+H)⁺.

### (2S,4R)-benzyl 1-((S)-3,3-dimethyl-2-((phenoxycarbonyl)amino)butanoyl)-4-hydroxypyrrolidine-2-carboxylate (Intermediate KB)

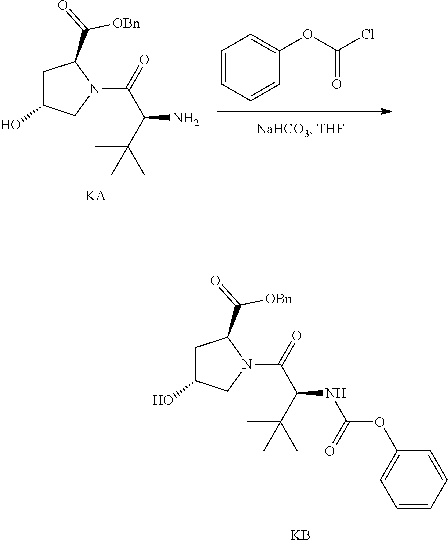

To a solution of benzyl (2S,4R)-1-[(2S)-2-amino-3,3-dimethyl-butanoyl]-4-hydroxy-pyrrolidine-2-carboxylate (3 g, 8.09 mmol, HCl, Intermediate KA) in THF (100 mL) was added NaHCO₃ (2.72 g, 32.36 mmol) in H₂O (10 mL) and phenyl carbonochloridate (1.52 g, 9.71 mmol) in THF (10 mL). The mixture was stirred at −10° C. for 0.5 hr. On completion, the reaction mixture was quenched with H₂O (100 mL) at 25° C., and then diluted with EA (100 mL) and extracted with EA (50 mL×3). The combined organic layers were washed with NaCl (50 mL×3), dried over Na₂SO₄, filtered and concentrated in vacuo to give a residue. The residue was purified by column chromatography (SiO₂, Petroleum ether/Ethyl acetate=10/1 to 1/1) to give the title compound (2 g, 54% yield) as a white oil. ¹H NMR (400 MHz, DMSO-d6) δ ppm 0.96 (s, 9H) 1.96-1.88 (m, 1H) 2.14 (m, 1H) 3.72-3.59 (m, 2H) 4.22 (d, J=9.26 Hz, 1H) 4.32 (s, 1H) 4.44 (t, J=8.38 Hz, 1H) 5.18-5.08 (m, 2H) 5.21 (d, J=3.88 Hz, 1H) 7.10 (d, J=8.8 Hz, 2H) 7.23-7.17 (m, 1H) 7.41-7.30 (m, 7H) 7.86 (d, J=9.13 Hz, 1H). LC-MS (ESI⁺) m/z 455.1 (M+H)⁺.

### (2S,4R)-4-hydroxy-1-((S)-2-(6-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxamido)-3,3-dimethylbutanoyl)pyrrolidine-2-carboxylic acid (Intermediate KC)

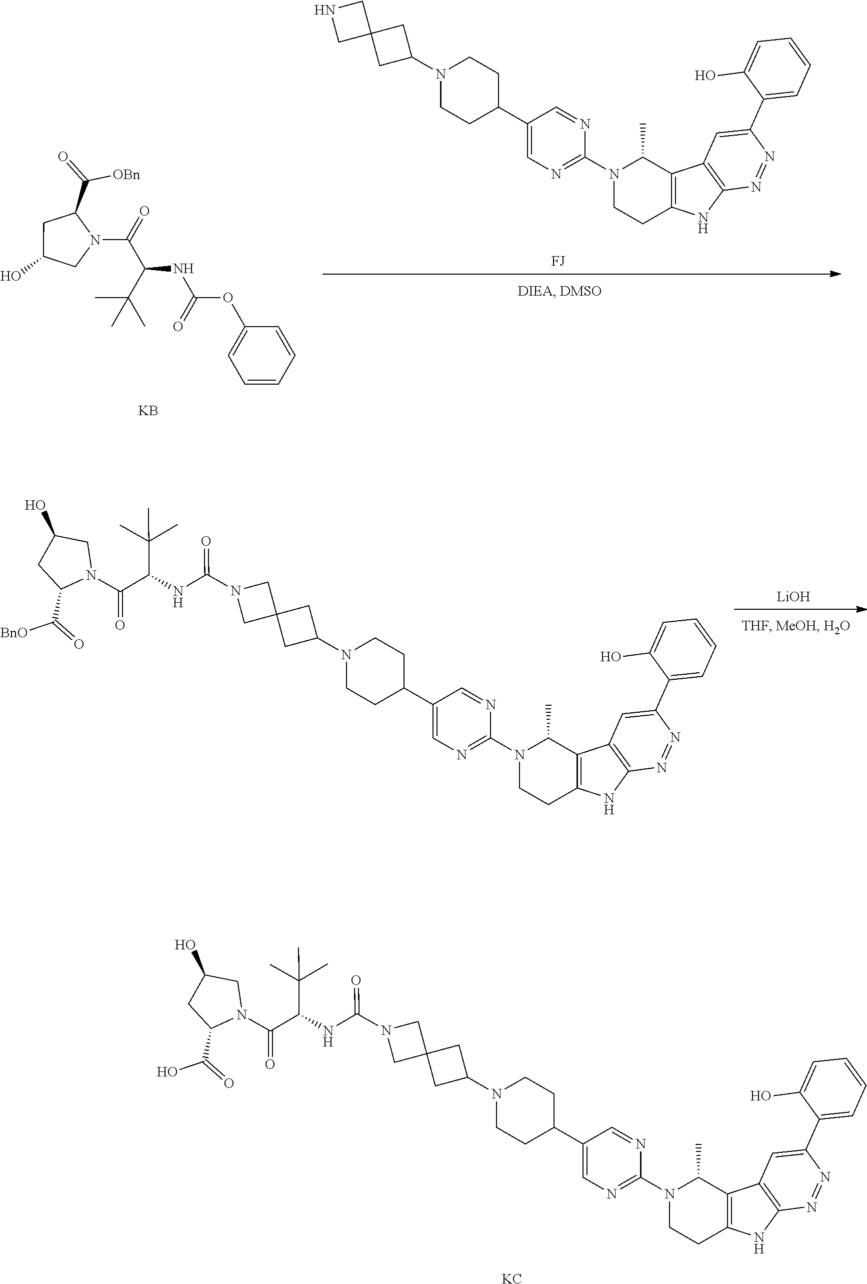

Step 1-(2S,4R)-benzyl 4-hydroxy-1-((S)-2-(6-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxamido)-3,3-dimethylbutanoyl)pyrrolidine-2-carboxylate. To a solution of benzyl (2S,4R)-1-[(2S)-3,3-dimethyl-2-(phenoxycarbonylamino)butanoyl]-4-hydroxy-pyrrolidine-2-carboxylate (500 mg, 1.10 mmol, Intermediate KB) and (R)-2-(6-(5-(1-(2-azaspiro[3.3]heptan-6-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (769 mg, 1.32 mmol, FA, Intermediate FJ) in DMSO (8 mL) was added DIEA (426.53 mg, 3.30 mmol) and the reaction mixture was stirred at 70° C. for 12 hrs. On completion, the reaction mixture was filtered to give a residue. The crude product was purified by reversed-phase HPLC (0.1% FA condition). After prep. HPLC purification, the eluent was concentrated or evaporated to remove the organic solvents. The residual aqueous solution was lyophilized to give the title compound as a yellow solid (0.6 g, 57% yield). LC-MS (ESI⁺) m/z 897.5 (M+H)⁺.

Step 2-(2S,4R)-4-hydroxy-1-((S)-2-(6-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxamido)-3,3-dimethylbutanoyl)pyrrolidine-2-carboxylic acid. To a solution of benzyl (2S,4R)-4-hydroxy-1-[(2S)-2-[[6-[4-[2-[(3R)-12-(2-hydroxyphenyl)-3-methyl-4,8,10,11-tetrazatricyclo[7.4.0.02,7]trideca-1(9),2(7),10,12-tetraen-4-yl]pyrimidin-5-yl]-1-piperidyl]-2-azaspiro[3.3]heptane-2-carbonyl]amino]-3,3-dimethyl-butanoyl]pyrrolidine-2-carboxylate (0.42 g, 445.34 umol, FA) in THF (1.3 mL), MeOH (1.3 mL) and H2O (1.3 mL) was added LiOH·H2O (93.44 mg, 2.23 mmol). The mixture was then stirred at 25° C. for 2 hr. On completion, the pH was adjusted to around 6 by progressively adding FA. The crude product was purified by reversed-phase HPLC (0.1% FA condition). After prep. HPLC purification, the eluent was concentrated or evaporated to remove the organic solvents. The residual aqueous solution was lyophilized to give the title compound as a yellow solid (0.3 g, 78% yield). ¹H NMR (400 MHz, DMSO-d₆) δ ppm 0.93 (s, 9H) 1.56-1.48 (m, 3H) 1.65-1.56 (m, 2H) 1.79-1.67 (m, 4H) 1.96-1.87 (m, 3H) 2.14-2.02 (m, 2H) 2.29-2.20 (m, 2H) 2.39-2.30 (m, 2H) 2.70-2.65 (m, 1H) 2.97-2.82 (m, 4H) 3.62 (s, 2H) 3.78-3.69 (m, 2H) 3.88-3.79 (m, 2H) 4.26-4.20 (m, 1H) 4.33-4.28 (m, 2H) 4.48 (s, 1H) 5.08-5.01 (m, 1H) 5.64-5.58 (m, 1H) 6.07-6.00 (m, 1H) 6.99-6.92 (m, 2H) 7.34-7.27 (m, 2H) 8.20 (br d, J=7.00 Hz, 1H) 8.31 (s, 2H) 8.70 (s, 1H) 12.27 (s, 1H); LC-MS (ESI⁺) m/z 807.8 (M+H)⁺.

### (2-(2-((tert-butyldimethylsilyl)oxy)ethoxy)-4-ethynylphenyl)methanamine (Intermediate KD)

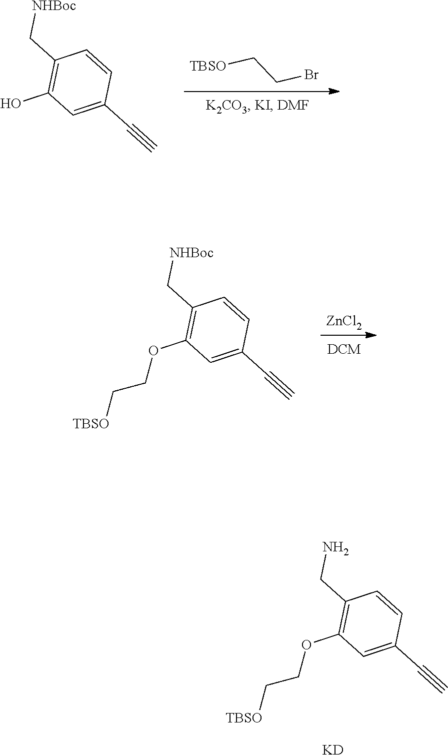 [SMILES: C#Cc1ccc(CNC(=O)OC(C)(C)C)c(O)c1 ; CC(C)(C)[Si](C)(C)OCCBr ; C#Cc1ccc(CNC(=O)OC(C)(C)C)c(OCCO[Si](C)(C)C(C)(C)C)c1 ; "ZnCl2" ; C#Cc1ccc(CN)c(OCCO[Si](C)(C)C(C)(C)C)c1 ; "KD"]

Step 1-Tert-butyl 2-(2-((tert-butyldimethylsilyl)oxy)ethoxy)-4-ethynylbenzylcarbamate. To a solution of tert-butyl N-[(4-ethynyl-2-hydroxy-phenyl)methyl]carbamate (1 g, 4 mmol, synthesized via Steps 1-3 of Intermediate HY) in DMF (25 mL) was added K₂CO₃ (1.68 g, 12.13 mmol), KI (134.26 mg, 808.77 umol) and 2-bromoethoxy-tert-butyl-dimethyl-silane (1.45 g, 6.07 mmol, CAS #86864-60-0). The mixture was stirred at 75° C. for 2 hr. On completion, the reaction mixture was quenched with sat. NH₄Cl (10 mL) at 25° C., and then diluted with EA (10 mL) and extracted with EA (10 mL×3). The combined organic layers were washed with NaCl (10 mL×3), dried over Na₂SO₄, filtered and concentrated under reduced pressure to give a residue. The crude product was purified by column chromatography (SiO₂, Petroleum ether/Ethyl acetate=100/1 to 20/1) to give the title compound (1 g, 60% yield) as a white oil. ¹H NMR (400 MHz, CDCl₃-d) δ ppm 0.11 (s, 6H) 0.92 (s, 9H) 1.44 (s, 9H) 3.06 (s, 1H) 3.99-3.96 (m, 2H) 4.08-4.06 (m, 2H) 4.29 (d, J=1.00 Hz, 2H) 5.04 (s, 1H) 6.99 (s, 1H) 7.08 (dd, J=7.63, 1.13 Hz, 1H) 7.22 (d, J=1.00 Hz, 1H); LC-MS (ESI+) m/z 428.2 (M+Na)⁺.

Step 2-(2-(2-((tert-butyldimethylsilyl)oxy)ethoxy)-4-ethynylphenyl)methanamine. To a solution of tert-butyl N-[[2-[2-[tert-butyl(dimethyl)silyl]oxyethoxy]-4-ethynyl-phenyl]methyl]carbamate (0.9 g, 2 mmol) in DCM (9 mL) was added ZnCl₂ (604.86 mg, 4.44 mmol). The mixture was then stirred at 25° C. for 12 hr. On completion, the reaction mixture was filtered and concentrated under reduced pressure to give a residue. The crude product was purified by column chromatography (SiO₂, DCM/MeOH=40/1 to 10/1) to give the title compound (0.5 g, 71% yield) as a white solid. ¹H NMR (400 MHz, CDCl₃-d) δ ppm 0.12 (s, 6H) 0.90 (s, 9H) 3.11 (s, 1H) 4.01 (t, J=4.88 Hz, 2H) 4.23-4.13 (m, 4H) 7.05 (d, J=0.88 Hz, 1H) 7.12-7.08 (m, 1H) 7.34 (br d, J=7.50 Hz, 1H); LC-MS (ESI+) m/z 306.1 (M+H)⁺.

### Methyl 4-(2-(aminomethyl)-5-ethynylphenoxy)butanoate (Intermediate KE)

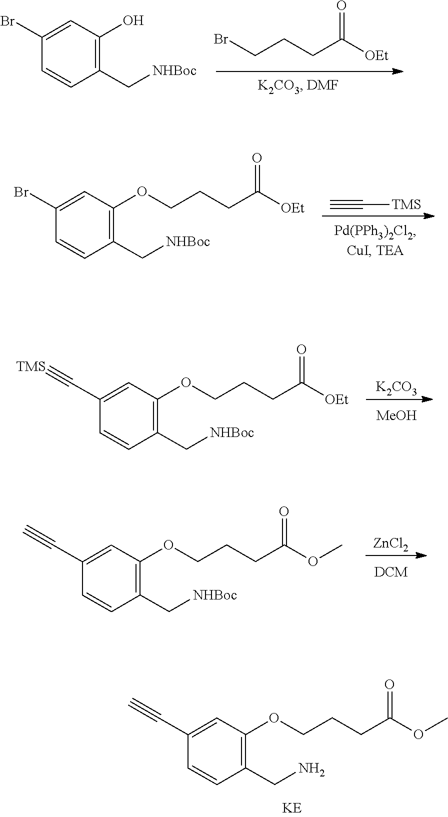

Step 1-Ethyl 4-(5-bromo-2-(((tert-butoxycarbonyl)amino)methyl)phenoxy)butanoate. A mixture of tert-butyl N-[(4-bromo-2-hydroxy-phenyl)methyl]carbamate (3 g, 9.93 mmol, synthesized via Step 1 of Intermediate HY), ethyl 4-bromobutanoate (5.81 g, 29.8 mmol, CAS #2969-81-5), K₂CO₃ (4.12 g, 29.8 mmol), and KI (330 mg, 1.99 mmol) in DMF (30 mL) was degassed and purged with N₂ three times. Then the mixture was stirred at 75° C. for 2 hours under N₂ atmosphere. On completion, the reaction mixture was quenched by addition water (30 mL) at 25° C., and then diluted with ethyl acetate (10 mL) and extracted with ethyl acetate (30 mL×3). The combined organic layers were washed with brine (40 mL×5), dried over Na₂SO₄, filtered and concentrated under reduced pressure to give a residue. The residue was purified by column chromatography (SiO₂, Petroleum ether/Ethyl acetate=I/O to 5/1) to give the title compound (4 g, 88% yield) as a white oil. LC-MS (ESI⁺) m/z 315.9(M−100+H)⁺.

Step 2-Ethyl 4-(2-(((tert-butoxycarbonyl)amino)methyl)-5-((trimethylsilyl)ethynyl)phenoxy)butanoate. A mixture of ethyl 4-[5-bromo-2-[(tert-butoxycarbonylamino)methyl]phenoxy]butanoate (3.6 g, 8.65 mmol), ethynyl(trimethyl)silane (8.49 g, 86.5 mmol, CAS #1066-54-2), and CuI (329 mg, 1.73 mmol) in TEA (18 mL) was degassed and purged with N₂ three times. Then Pd(PPh₃)₂Cl₂ (607 mg, 865 umol) was added and the mixture was stirred at 80° C. for 12 hrs under N₂ atmosphere. On completion, the mixture was filtered. The residue was purified by column chromatography (SiO₂, Petroleum ether/Ethyl acetate=1/0 to 20/1) to give the title compound (3.3 g, 85% yield) as a yellow oil. ¹H NMR (400 MHz, DMSO-d₆) δ 7.72 (t, J=5.6 Hz, 1H), 7.63 (d, J=7.6 Hz, 1H), 7.57-7.52 (m, 1H), 7.48 (s, 1H), 4.62-4.51 (m, 6H), 3.84 (s, 1H), 3.01-2.97 (m, 2H), 1.91 (s, 9H), 1.81 (s, 1H), 1.70 (t, J=7.2 Hz, 3H), 0.74 (s, 9H); LC-MS (ESI⁺) m/z 317.2(M−116+H)⁺.

Step 3-Methyl 4-(2-(((tert-butoxycarbonyl)amino)methyl)-5-ethynylphenoxy)butanoate. To a solution of ethyl 4-[2-[(tert-butoxycarbonylamino)methyl]-5-(2-trimethylsilylethynyl)phenoxy]butanoate (3.7 g, 8.53 mmol) in MeOH (37 mL) was added K₂CO₃ (2.36 g, 17.1 mmol). The mixture was stirred at 25° C. for 30 mins. On completion, the reaction mixture was concentrated under reduced pressure to remove EA. The residue was diluted with water (20 mL) and extracted with EA (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=1/0 to 15/1) to give the title compound (2.8 g, 94% yield) as a white solid. ¹H NMR (400 MHz, CDCl₃-d) δ 7.26 (d, J=7.6 Hz, 1H), 7.12 (d, J=7.6 Hz, 1H), 6.99 (s, 1H), 5.09-4.94 (m, 1H), 4.33 (d, J=5.2 Hz, 2H), 4.08 (t, J=6.0 Hz, 2H), 3.75 (s, 3H), 3.10 (s, 1H), 2.58 (t, J=7.2 Hz, 2H), 2.20 (quin, J=6.8 Hz, 2H), 1.49 (s, 9H); LC-MS (ESI⁺) m z 369.9 (M+23)⁺.

Step 4-Methyl 4-(2-(aminomethyl)-5-ethynylphenoxy)butanoate. To a solution of methyl 4-[2-[(tert-butoxycarbonylamino) methyl]-5-ethynyl-phenoxy] butanoate (500 mg, 1.44 mmol) in DCM (5 mL) was added ZnCl₂ (392 mg, 2.88 mmol). The mixture was stirred at 25° C. for 12 hrs. The reaction mixture was filtered and concentrated under reduced pressure to give a residue. The residue was purified by column chromatography (SiO₂, DCM: MeOH=1/0 to 5/1) to give the title compound (400 mg, 96% yield) as a white solid. LC-MS (ESI⁺) m/z 231 (M-16)⁺.

### (R)-2-((tert-butyldimethylsilyl)oxy)-1-(4-ethynylphenyl)ethanamine (Intermediate KF)

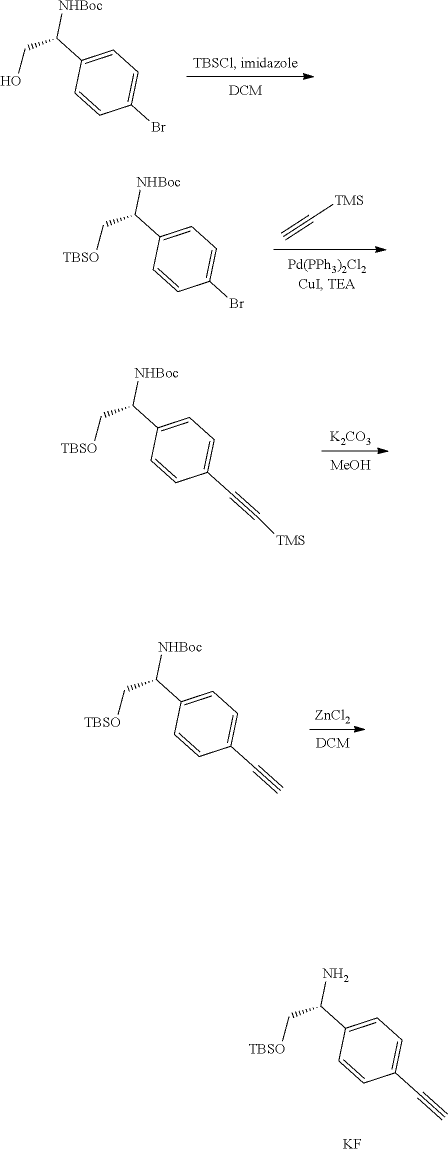

(R)-tert-butyl (1-(4-bromophenyl)-2-hydroxyethyl)carbamate. To a solution of tert-butyl N-[(1R)-1-(4-bromophenyl)-2-hydroxy-ethyl]carbamate (3.9 g, 12.33 mmol, CAS #3601-66-9) in DCM (40 mL) was added TBSCl (2.04 g, 13.57 mmol, 1.66 mL) and imidazole (1.68 g, 24.7 mmol). The mixture was stirred at 25° C. for 12 hr. On completion, the reaction mixture was quenched with water (40 mL), and extracted with EA (80 mL×3). The combined organic layers were washed with brine (80 mL), dried over anhydrous Na₂SO₄, filtered and concentrated under reduced pressure to afford the title compound (6.22 g).

Step 2-(R)-tert-butyl (2-((tert-butyldimethylsilyl)oxy)-1-(4-((trimethylsilyl)ethynyl)phenyl)ethyl)carbamate. To a solution of tert-butyl N-[(1R)-1-(4-bromophenyl)-2-[tert-butyl(dimethyl)silyl]oxy-ethyl]carbamate (6.13 g, 14.2 mmol) in TEA (60 mL) was added ethynyl(trimethyl)silane (3.50 g, 35.6 mmol, 4.93 mL), dichloropalladium;triphenylphosphane (999.56 mg, 1.42 mmol) and CuI (271.22 mg, 1.42 mmol). The mixture was stirred at 65° C. for 12 hr. On completion, the reaction mixture was quenched with sat. aq NH4Cl 40 mL, and then extracted with EA (100 mL×3). The combined organic layers were washed with brine (60 mL), dried over anhydrous Na₂SO₄, filtered and concentrated under reduced pressure to give a residue. The residue was purified by column chromatography (SiO2, Petroleum ether/Ethyl acetate=100/1 to 50/1) to give the title compound (3.1 g, 44% yield as a yellow solid. LC-MS (ESI+) m/z 349.2 (M+H+H2O)⁺.

Step 3-(R)-tert-butyl (2-((tert-butyldimethylsilyl)oxy)-1-(4-ethynylphenyl)ethyl)carbamate. To a solution of tert-butyl N-[(1R)-2-[tert-butyl(dimethyl)silyl]oxy-1-[4-(2-trimethylsilylethynyl)phenyl]ethyl]carbamate (1 g, 2 mmol) in MeOH (10 mL) was added K₂CO₃ (370.40 mg, 2.68 mmol). Then the mixture was stirred at 25° C. for 1.5 hr. On completion, the mixture was filtered. The filtrate was washed with sat.aq NH₄Cl (5 mL), then with brine (10 mL), dried over anhydrous Na₂SO₄, filtered and concentrated under reduced pressure to give the title compound (950 mg, 24% yield) as a yellow solid. LC-MS (ESI+) m/z320.2 (M+H−C4H9)⁺.

Step 4-(R)-2-((tert-butyldimethylsilyl)oxy)-1-(4-ethynylphenyl)ethanamine. To a solution of tert-butyl N-[(1R)-2-[tert-butyl(dimethyl)silyl]oxy-1-(4-ethynylphenyl)ethyl]carbamate (450 mg, 1.20 mmol) in DCM (3 mL) was added ZnCl₂ (326.61 mg, 2.40 mmol, 112.24 uL). The mixture was then stirred at 25° C. for 12 hr. On completion, the mixture was filtered. The filtrate was washed with H₂O (3 mL), then with brine (3 mL), dried over anhydrous Na₂SO₄, filtered and concentrated under reduced pressure to give a residue. The residue was purified by column chromatography (SiO₂, Petroleum ether:Ethyl acetate:Triethylamine=1:1:0.05) to give title compound (300 mg, 79% yield) as a yellow solid. LC-MS (ESI+) m/z276.2 (M+H)⁺.

### (S)-[1,4′-bipiperidin]-4-yl(2-(2-hydroxyphenyl)-6a,7,9,10-tetrahydro-5H-pyrazino[1′,2′:4,5]pyrazino[2,3-c]pyridazin-8(6H)-yl)methanone (Intermediate KG)

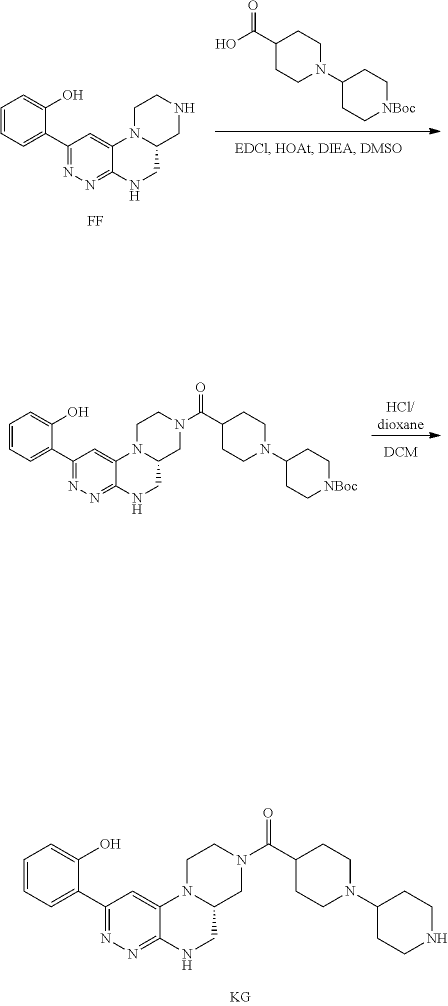 [SMILES: Oc1ccccc1-c1cc2c(nn1)NC[C@H]1CNCCN21 ; CC(C)(C)OC(=O)N1CCC(N2CCC(C(=O)O)CC2)CC1 ; "FF" ; CC(C)(C)OC(=O)N1CCC(N2CCC(C(=O)N3CCN4c5cc(-c6ccccc6O)nnc5NC[C@H]4C3)CC2)CC1 ; O=C(C1CCN(C2CCNCC2)CC1)N1CCN2c3cc(-c4ccccc4O)nnc3NC[C@H]2C1]

Step 1-(S)-tert-butyl 4-(2-(2-hydroxyphenyl)-6,6a,7,8,9,10-hexahydro-5H-pyrazino[1′,2′:4,5]pyrazino[2,3-c]pyridazine-8-carbonyl)-[1,4′-bipiperidine]-1′-carboxylate. To a solution of 2-[(1OR)-1,5,6,8,12-pentazatricyclo[8.4.0.02,7]tetradeca-2,4,6-trien-4-yl]phenol (200 mg, 706 umol, Intermediate FF) in DMSO (6 mL) was added HOAt (144 mg, 1.06 mmol, 148 uL), EDCI (203 mg, 1.06 mmol), DIEA (547 mg, 4.24 mmol, 738 uL) and 1-(1-tert-butoxycarbonyl-4-piperidyl)piperidine-4-carboxylic acid (264 mg, 847 umol, CAS #201810-59-5). The mixture was then stirred at 25° C. for 2 hours. On completion, the reaction mixture was extracted with DCM (30 mL×3). The combined organic layers were washed with aqueous NaCl (30 mL×3), dried over Na₂SO₄ and then concentrated under reduced pressure to give the title compound (100 mg,) as a yellow solid. LC-MS (ESI⁺) m/z 578.4 (M+H)⁺.

Step 2-(S)-[1,4′-bipiperidin]-4-yl(2-(2-hydroxyphenyl)-6a,7,9,10-tetrahydro-5H-pyrazino[1′,2′:4,5]pyrazino[2,3-c]pyridazin-8(6H)-yl)methanone. To a solution of tert-butyl 4-[4-[(10S)-4-(2-hydroxyphenyl)-1,5,6,8,12-pentazatricyclo[8.4.0.02,7]tetradeca-2,4,6-triene-12-carbonyl]-1-piperidyl]piperidine-1-carboxylate (100 mg, 173 umol) in DCM (2 mL) was added HCl/dioxane (5 M, 173 uL). The mixture was then stirred at 25° C. for 2 hrs. On completion, the reaction mixture was concentrated under reduced pressure to give the title compound (144 mg) as a red solid. LC-MS (ESI⁺) m/z 478.4 (M+H)⁺.

### (S)-tert-butyl 3-(2-(2-hydroxyphenyl)-6,6a,7,8,9,10-hexahydro-5H-pyrazino[1′,2′:4,5]pyrazino[2,3-c]pyridazine-8-carbonyl)azetidine-1-carboxylate (Intermediate KH)

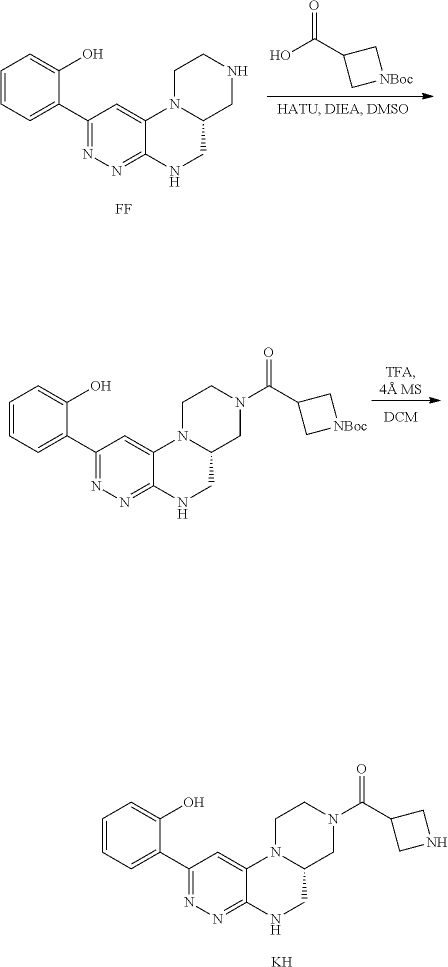

Step 1-tert-butyl (S)-3-(2-(2-hydroxyphenyl)-6,6a,7,8,9,10-hexahydro-5H-pyrazino[1′,2′:4,5]pyrazino[2,3-c]pyridazine-8-carbonyl)azetidine-1-carboxylate. To a solution of 2-[(1OR)-1,5,6,8,12-pentazatricyclo[8.4.0.0^{2,7}]tetradeca-2,4,6-trien-4-yl]phenol (1.39 g, 4.35 mmol, Intermediate FF) and 1-tert-butoxycarbonylazetidine-3-carboxylic acid (673 mg, 3.34 mmol, CAS #142253-55-2) in DMSO (10 mL) was added DIEA (1.73 g, 13.4 mmol) EDCI (1.60 g, 8.36 mmol) and HOAt (1.14 g, 8.36 mmol). The mixture was then stirred at 0-25° C. for 1 hr. On completion, the reaction mixture was concentrated under reduced pressure to remove solvent. The crude product was purified by reversed-phase HPLC (0.1% FA condition) to give the title compound (1.25 g, 61% yield, FA) as a yellow solid.LC-MS (ESI⁺) m/z 467.0 (M+H).

Step 2-(S)-tert-butyl 3-(2-(2-hydroxyphenyl)-6,6a,7,8,9,10-hexahydro-5H-pyrazino[1′,2′:4,5]pyrazino[2,3-c]pyridazine-8-carbonyl)azetidine-1-carboxylate. To a solution of tert-butyl 3-[(10S)-4-(2-hydroxyphenyl)-1,5,6,8,12-pentazatricyclo[8.4.0.0^{2,7}]tetradeca-2,4,6-triene-12-carbonyl]azetidine-1-carboxylate (1.25 g, 2.68 mmol) in DCM (20 mL) was added TFA (7.70 g, 67.5 mmol) and 4 Å molecular sieves (1.25 g, 1.25 mol). The mixture was then stirred at 25° C. for 1 hr. On completion, the reaction mixture was concentrated under reduced pressure to remove DCM. The crude product was purified by reversed-phase HPLC(0.1% FA condition) to give the title compound (700 mg, 54% yield, FA) as a yellow solid.LC-MS (ESI⁺) m/z 367.1 (M+H).

### (S)-tert-butyl 4-(3-(2-(2-hydroxyphenyl)-6,6a,7,8,9,10-hexahydro-5H-pyrazino[1′,2′:4,5]pyrazino[2,3-c]pyridazine-8-carbonyl)azetidin-1-yl)piperidine-1-carboxylate (Intermediate KI)

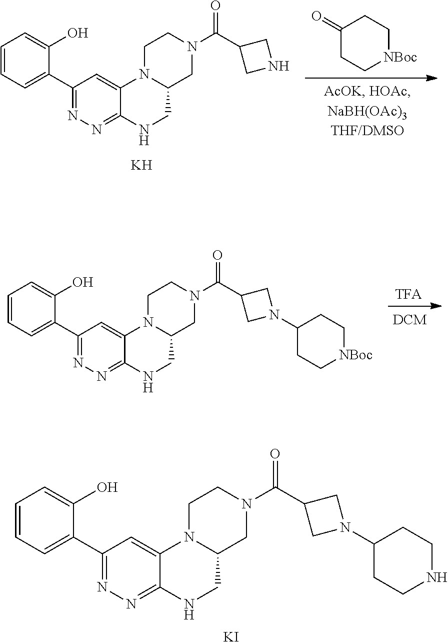

Step 1-(S)-azetidin-3-yl(2-(2-hydroxyphenyl)-6a,7,9,10-tetrahydro-5H-pyrazino[1′,2′:4,5]pyrazino[2,3-c]pyridazin-8(6H)-yl)methanone. To a solution of azetidin-3-yl-[(10S)-4-(2-hydroxyphenyl)-1,5,6,8,12-pentazatricyclo[8.4.0.0^{2,7}]tetradeca-2,4,6-trien-12-yl]methanone (300 mg, 600 umol, Intermediate KH) in DMSO (0.5 mL) and THF (4 mL) added KOAc (184 mg, 1.87 mmol) and 4 Å molecular sieves (300 mg, 624 umol) stirred for 15 minutes. Then tert-butyl 4-oxopiperidine-1-carboxylate (161.74 mg, 811.76 umol, CAS #79099-07-3) and HOAc (112 mg, 1.87 mmol) was added for 15 minutes. Then NaBH(OAc)₃ (330.86 mg, 1.56 mmol) was added at 0° C., then the mixture was stirred at 0-25° C. for 1 hr. On completion, the reaction mixture was concentrated under reduced pressure to remove THF. The crude product was purified by reversed-phase HPLC (0.1% FA condition) to give the title compound (150 mg, 36% yield, FA) as a white solid. LC-MS (ESI⁺) m/z 550.3 (M+H).

Step 2-(S)-tert-butyl 4-(3-(2-(2-hydroxyphenyl)-6,6a,7,8,9,10-hexahydro-5H-pyrazino[1′,2′:4,5]pyrazino[2,3-c]pyridazine-8-carbonyl)azetidin-1-yl)piperidine-1-carboxylate. To a solution of tert-butyl 4-[3-[(10S)-4-(2-hydroxyphenyl)-1,5,6,8,12-pentazatricyclo[8.4.0.0^{2,7}]tetradeca-2,4,6-triene-12-carbonyl]azetidin-1-yl]piperidine-1-carboxylate (300 mg, 546 umol) in DCM (5 mL) was added TFA (1.54 g, 13.5 mmol) and 4 Å molecular sieves (300 mg, 546 umol). The mixture was then stirred at 25° C. for 3 hr. On completion, the reaction mixture was concentrated under reduced pressure to remove DCM. The crude product was purified by reversed-phase HPLC (0.1% FA condition) to give the title compound (244 mg, 88% yield, FA) as a yellow solid. LC-MS (ESI⁺) m/z 450.3 (M+H).

### (R)-2-(2-(methoxymethoxy)phenyl)-6,6a,7,8,9,10-hexahydro-5H-pyrazino[1′,2′:4,5]pyrazino[2,3-c]pyridazine (Intermediate KJ)

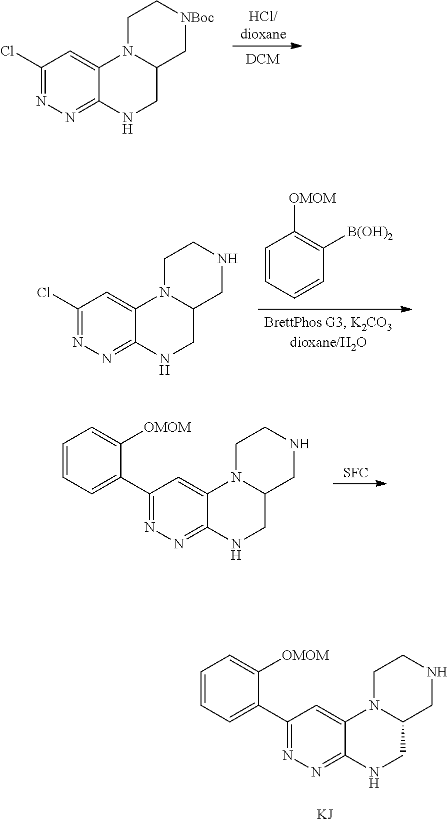

Step 1-2-Chloro-6,6a,7,8,9,10-hexahydro-5H-pyrazino[1′,2′:4,5]pyrazino[2,3-c]pyridazine. To a solution of tert-butyl 4-chloro-1,5,6,8,12-pentazatricyclo[8.4.0.0^{2,7}]tetradeca-2,4,6-triene-12-carboxylate (11 g, 33.8 mmol, synthesized via Steps 1-2 of Intermediate FD) in DCM (50 mL) was added HCl/dioxane (10 M, 33.8 mL). The mixture was then stirred at 25° C. for 1 hour. On completion, the reaction mixture was concentrated under reduced pressure to give the title compound (11 g) as a yellow solid. LC-MS (ESI⁺) m/z 226.2 (M+H)⁺.

Step 2-2-(2-(Methoxymethoxy)phenyl)-6,6a,7,8,9,10-hexahydro-5H-pyrazino[1′,2′:4,5]pyrazino[2,3-c]pyridazine. To a solution of 4-chloro-1,5,6,8,12-pentazatricyclo[8.4.0.0^{2,7}]tetradeca-2,4,6-triene (9 g, 39.8 mmol) in dioxane (200 mL) H₂O (40 mL) was added BrettPhos Pd G3 (3.62 g, 3.99 mmol) and K₂CO₃ (27.6 g, 199 mmol) and [2-(methoxymethoxy)phenyl]boronic acid (21.8 g, 119 mmol). The mixture was stirred at 100° C. for 12 hours under N₂ atmosphere. On completion, the reaction mixture was diluted with water (40 mL) and extracted with DCM (40 mL×3). The combined organic layers were washed with aqueous NaCl (40 mL×3), dried over Na₂SO₄, filtered and concentrated under reduced pressure to give a residue. The residue was purified by pre-HPLC (NH₃H₂O) to give the title compound (5.1 g, 41% yield) as a yellow solid. LC-MS (ESI⁺) m/z 328.0 (M+H)⁺.

Step 3-(R)-2-(2-(methoxymethoxy)phenyl)-6,6a,7,8,9,10-hexahydro-5H-pyrazino[1′,2′:4,5]pyrazino[2,3-c]pyridazine. 2-(2-(Methoxymethoxy)phenyl)-6,6a,7,8,9,10-hexahydro-5H-pyrazino[1′,2′:4,5]pyrazino[2,3-c]pyridazine (3.5 g) was purified by SFC (column: Phenomenex-Cellulose-2 (250 mm*30 mm, 10 um); mobile phase: [0.1% NH₃H₂O MEOH]; B %: 45%-45%, 7; 2000 min) to give (R)-2-(2-(methoxymethoxy)phenyl)-6,6a,7,8,9,10-hexahydro-5H-pyrazino[1′,2′:4,5]pyrazino[2,3-c]pyridazine (1.3 g, 31% yield) as a yellow solid. Retention time: 2.493; LC-MS (ESI⁺) m/z 328.3 (M+H)⁺.

### (S)-tert-butyl 4-(4-(2-(2-(methoxymethoxy)phenyl)-6a,7,9,10-tetrahydro-5H-pyrazino[1′,2′:4,5]pyrazino[2,3-c]pyridazin-8(6H)-yl)phenyl)piperidine-1-carboxylate (Intermediate KK)

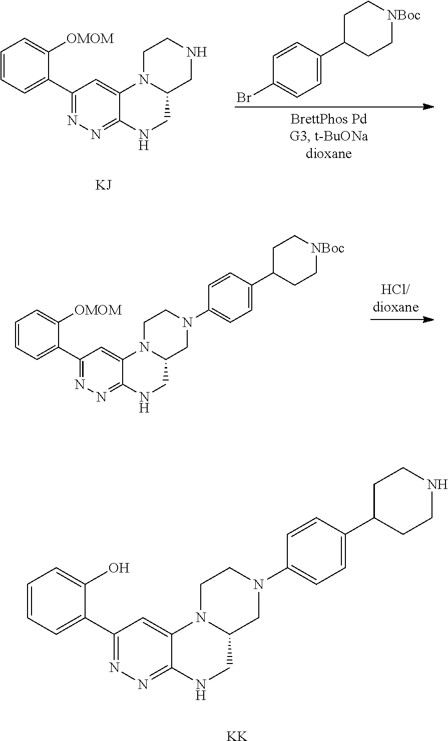

Step 1-(S)-tert-butyl 4-(4-(2-(2-(methoxymethoxy)phenyl)-6a,7,9,10-tetrahydro-5H-pyrazino[1′,2′:4,5]pyrazino[2,3-c]pyridazin-8(6H)-yl)phenyl)piperidine-1-carboxylate. To a solution of (10R)-4-[2-(methoxymethoxy)phenyl]-1,5,6,8,12-pentazatricyclo[8.4.0.0^{2,7}]tetradeca-2,4,6-triene (200 mg, 611 umol, Intermediate KJ) in dioxane (5 mL) was added BrettPhos Pd G3 (55.4 mg, 61 umol) and t-BuONa (2 M, 916 uL), tert-butyl 4-(4-bromophenyl)piperidine-1-carboxylate (249 mg, 733 umol, CAS #769944-78-7). The mixture was stirred at 110° C. for 2 hours under N₂ atmosphere by microwave under 15 psi. On completion, the reaction mixture was diluted with DMSO (1 ml). The residue was purified by prep-HPLC (FA condition) to give the title compound (80 mg, 20% yield) as a white solid. LC-MS (ESI⁺) m/z 587.3 (M+H)⁺.

Step 2-(S)-tert-butyl 4-(4-(2-(2-(methoxymethoxy)phenyl)-6a,7,9,10-tetrahydro-5H-pyrazino[1′,2′:4,5]pyrazino[2,3-c]pyridazin-8(6H)-yl)phenyl)piperidine-1-carboxylate. To a solution of tert-butyl 4-[4-[(10R)-4-[2-(methoxymethoxy)phenyl]-1,5,6,8,12-pentazatricyclo[8.4.0.0^{2,7}]tetradeca-2,4,6-trien-12-yl]phenyl]piperidine-1-carboxylate (30 mg, 51.1 umol) was added HCl/dioxane (10 M, 25.5 uL). The mixture was stirred at 25° C. for 5 rs. On completion, the reaction mixture was concentrated under reduced pressure to give the title compound (30 mg) as a white solid. LC-MS (ESI⁺) m z 443.5(M+H)⁺.

Tert-butyl 4-(piperidin-4-ylmethyl)piperazine-1-carboxylate (Intermediate KL)

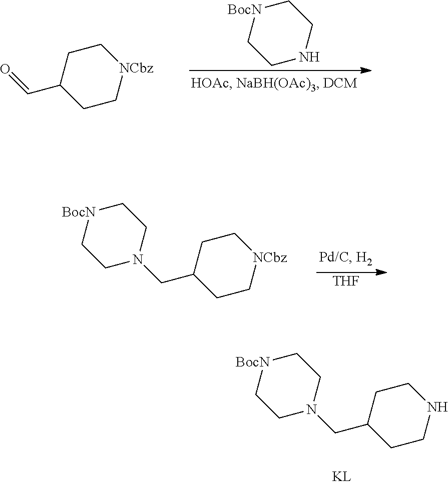 [SMILES: O=CC1CCN(C(=O)OCc2ccccc2)CC1 ; CC(C)(C)OC(=O)N1CCNCC1 ; CC(C)(C)OC(=O)N1CCN(CC2CCN(C(=O)OCc3ccccc3)CC2)CC1 ; CC(C)(C)OC(=O)N1CCN(CC2CCNCC2)CC1 ; "THF"]

Step 1-tert-butyl 4-((1-((benzyloxy)carbonyl)piperidin-4-yl)methyl)piperazine-1-carboxylate. To a solution of benzyl 4-formylpiperidine-1-carboxylate (4 g, 16.18 mmol, CAS #138163-08-3) in DCM (50 mL) was added AcOH (1.17 g, 19.4 mmol), and tert-butyl piperazine-1-carboxylate (3.62 g, 19.4 mmol, CAS #143238-38-4). Then NaBH(OAc)₃ (5.14 g, 24.2 mmol) was added at 0° C. and the mixture was stirred at 25° C. for 2 hours. On completion, the reaction mixture was diluted with water (2 mL), and then concentrated under reduced pressure to give the residue. The residue was purified by prep-HPLC (NH3·H2O condition) to give the title compound (9 g, 86% yield) as white solid. LC-MS (ESI⁺) m/z 418.3 (M+H)⁺.

Step 2-Tert-butyl 4-(piperidin-4-ylmethyl)piperazine-1-carboxylate. To a solution of tert-butyl 4-[(1-benzyloxycarbonyl-4-piperidyl)methyl]piperazine-1-carboxylate (7.4 g, 18 mmol) in THF (90 mL) was added Pd/C (7.4 g, 8.9 mmol, 10 wt %). The mixture was stirred at 25° C. for 12 hours under H₂ atmosphere. On completion, the reaction mixture was filtered and concentrated under reduced pressure to give the title compound (5.8 g) as a white solid. LC-MS (ESI⁺) m/z 284.3 (M+H)⁺.

### 2-(5-Methyl-6-(5-(4-(piperazin-1-ylmethyl)piperidin-1-yl)pyrimidin-2-yl)-6,7,8,9-tetrahydro-5H-pyrido[3′,4′:4,5]pyrrolo[2,3-c]pyridazin-3-yl)phenol (Intermediate KM)

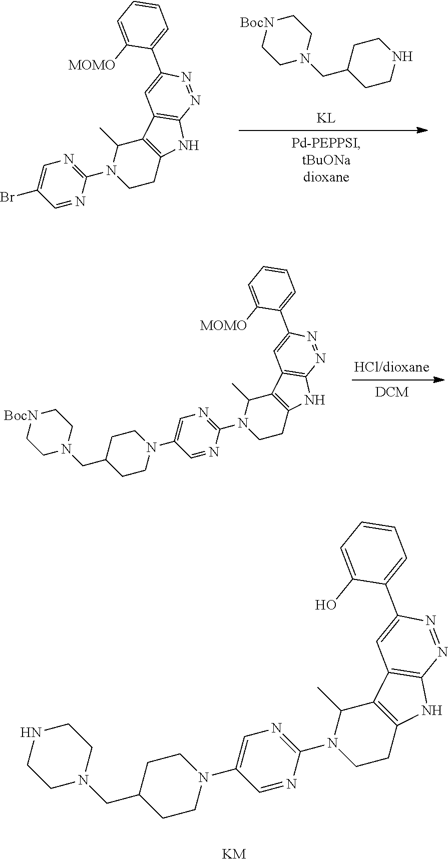

Step 1-Tert-butyl 4-((1-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)piperidin-4-yl)methyl)piperazine-1-carboxylate. To a solution of 4-(5-bromopyrimidin-2-yl)-12-[2-(methoxymethoxy)phenyl]-3-methyl-4,8,10,11-tetrazatricyclo[7.4.0.0^{2,7}]trideca-1(9),2(7),10,12-tetraene (600 mg, 1.25 mmol, synthesized via Step 1 of Intermediate GQ), tert-butyl 4-(4-piperidylmethyl)piperazine-1-carboxylate (423 mg, 1.50 mmol, Intermediate KL) in dioxane (6 mL) was added tBuONa (2 M, 1.87 mL) and PD-PEPPSI™-IPENT CATALYST (98.9 mg, 124 umol). The mixture was degassed and purged with N₂ three times, and then the mixture was stirred at 100° C. for 3 hours under N₂ atmosphere. On completion, the reaction mixture was filtered and concentrated under reduced pressure to give a residue. The residue was purified by prep-HPLC (NH₃.H₂O condition) to give the title compound (420 mg, 42% yield) as a yellow solid. LC-MS (ESI⁺) m/z 684.5 (M+H)⁺.

Step 2-2-(5-methyl-6-(5-(4-(piperazin-1-ylmethyl)piperidin-1-yl)pyrimidin-2-yl)-6,7,8,9-tetrahydro-5H-pyrido[3′,4′:4,5]pyrrolo[2,3-c]pyridazin-3-yl)phenol. To a solution of tert-butyl 4-[[1-[2-[12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-5-yl]-4-piperidyl]methyl]piperazine-1-carboxylate (420 mg, 614 umol) in DCM (1 mL) was added HCl/dioxane (4 M, 10 mL). The mixture was stirred at 25° C. for 0.5 hours. On completion, the reaction mixture was concentrated under reduced pressure to give the title compound (430 mg, HCl) as a yellow solid. LC-MS (ESI⁺) m/z 540.5 (M+H)⁺.

### 1-(2-(3-(2-Hydroxyphenyl)-5-methyl-7,8-dihydro-5H-pyrido[3′,4′:4,5]pyrrolo[2,3-c]pyridazin-6(9H)-yl)pyrimidin-5-yl)piperidine-4-carbaldehyde (Intermediate KN)

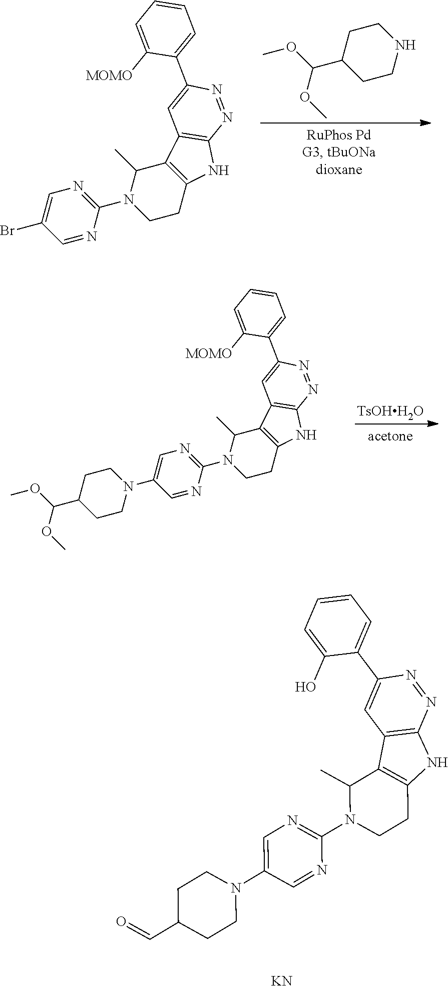

Step 1-6-(5-(4-(Dimethoxymethyl)piperidin-1-yl)pyrimidin-2-yl)-3-(2-(methoxymethoxy)phenyl)-5-methyl-6,7,8,9-tetrahydro-5H-pyrido[3′,4′:4,5]pyrrolo[2,3-c]pyridazine. To a solution of 4-(5-bromopyrimidin-2-yl)-12-[2-(methoxymethoxy)phenyl]-3-methyl-4,8,10,11-tetrazatricyclo[7.4.0.0^{2,7}]trideca-1(9),2(7),10,12-tetraene (1.00 g, 2.08 mmol, synthesized via Step 1 of Intermediate GQ), and 4-(dimethoxymethyl)piperidine (496 mg, 3.12 mmol, CAS #188646-83-5) in dioxane (10 mL) was added tBuONa (2 M, 5.19 mL) and RuPhos Pd G3 (173 mg, 207 umol). The mixture was degassed and purged with N₂ three times, and then the mixture was stirred at 100° C. for 12 hours. On completion, the reaction mixture was filtered and concentrated under reduced pressure to give a residue. The residue was purified by prep-HPLC (NH₃.H₂O condition) to give the title compound (550 mg, 44% yield) as a white solid. LC-MS (ESI⁺) m/z 560.4 (M+H)⁺.

Step 2-1-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidine-4-carbaldehyde. To a solution of 4-[5-[4-(dimethoxymethyl)-1-piperidyl]pyrimidin-2-yl]-12-[2-(methoxymethoxy)phenyl]-3-methyl-4,8,10,11-tetrazatricyclo[7.4.0.0^{2,7}]trideca-1(9),2(7),10,12-tetraene (1 g, 2 mmol) in H₂O (10 mL) and acetone (20 mL) was added TsOH.H₂O (679 mg, 3.57 mmol). The mixture was stirred at 70° C. for 2 hours. On completion, the reaction mixture was filtered and concentrated under reduced pressure to give a residue. The residue was purified by prep-HPLC (NH₃.H₂O condition) to give the title compound (250 mg, 21% yield) as a brown solid. LC-MS (ESI⁺) m/z 514.3 (M+H)+

### 2-(6-(5-(4-(2,6-Diazaspiro[3.3]heptan-2-ylmethyl)piperidin-1-yl)pyrimidin-2-yl)-5-methyl-6,7,8,9-tetrahydro-5H-pyrido[3′,4′:4,5]pyrrolo[2,3-c]pyridazin-3-yl)phenol (Intermediate KO)

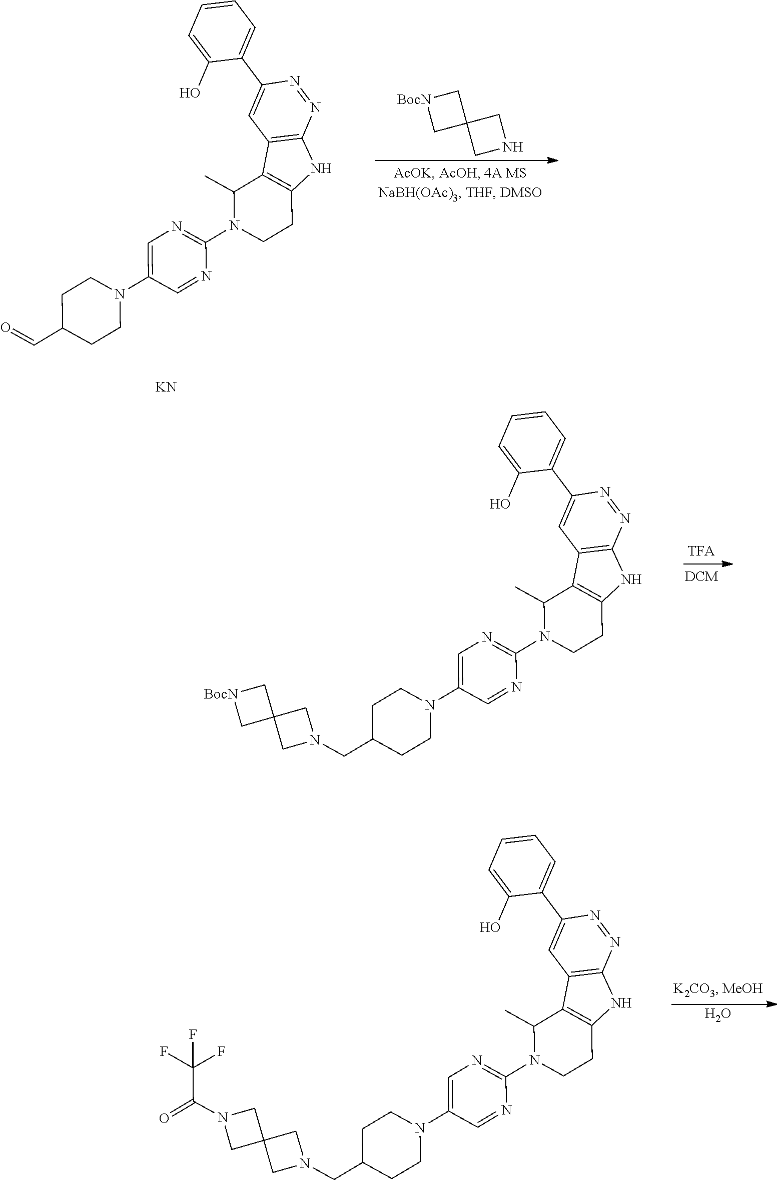 [SMILES: CC1c2c([nH]c3nnc(-c4ccccc4O)cc23)CCN1c1ncc(N2CCC(C=O)CC2)cn1 ; CC(C)(C)OC(=O)N1CC2(CNC2)C1 ; "KN" ; CC1c2c([nH]c3nnc(-c4ccccc4O)cc23)CCN1c1ncc(N2CCC(CN3CC4(C3)CN(C(=O)OC(C)(C)C)C4)CC2)cn1 ; CC1c2c([nH]c3nnc(-c4ccccc4O)cc23)CCN1c1ncc(N2CCC(CN3CC4(C3)CN(C(=O)C(F)(F)F)C4)CC2)cn1 ; O]

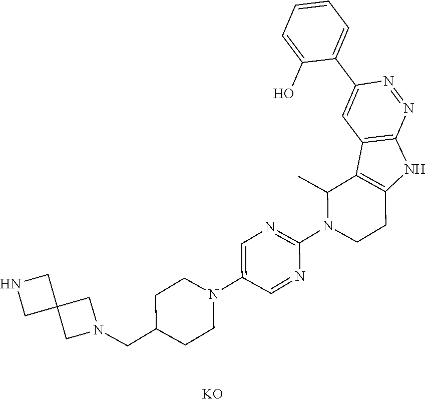 [SMILES: CC1c2c([nH]c3nnc(-c4ccccc4O)cc23)CCN1c1ncc(N2CCC(CN3CC4(CNC4)C3)CC2)cn1 ; "KO"]

Step 1-Tert-butyl 6-((1-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-4-yl)methyl)-2,6-diazaspiro[3.3]heptane-2-carboxylate. To a solution of tert-butyl 2,6-diazaspiro[3.3]heptane-2-carboxylate (150 mg, 584 umol, HOAC, CAS #1041026-70-3) in THF (2 mL) and DMSO (1 mL) was added AcOK (119 mg, 1.22 mmol) and 4 Å molecular sieves (400 mg). Then 1-[2-[12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-5-yl]piperidine-4-carbaldehyde (250 mg, 486 umol, Intermediate KN) and AcOH (73.1 mg, 1.22 mmol) was added. Then NaBH(OAc)₃ (309 mg, 1.46 mmol) was added at 0° C. The mixture was stirred at 25° C. for 2 hours. On completion, the reaction mixture was filtered and concentrated under reduced pressure to give a residue. The residue was purified by prep-HPLC (NH₃.H₂O condition) to give the title compound (230 mg, 65% yield) as a yellow solid. LC-MS (ESI⁺) m/z 696.3 (M+H)⁺.

Step 2-2,2,2-Trifluoro-1-(6-((1-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-4-yl)methyl)-2,6-diazaspiro[3.3]heptan-2-yl)ethanone. To a solution of tert-butyl 6-[[1-[2-[12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-5-yl]-4-piperidyl]methyl]-2,6-diazaspiro[3.3]heptane-2-carboxylate (230 mg, 330 umol) in DCM (2 mL) was added TFA (770 mg, 6.75 mmol). The mixture was stirred at 25° C. for 1 hour. On completion, the reaction mixture was concentrated under reduced pressure to remove DCM. The residue was purified by prep-HPLC (FA condition) to give the title compound (140 mg, 58% yield, FA) as a red solid. LC-MS (ESI⁺) m/z 648.3 (M+H)⁺.

Step 3-2-(6-(5-(4-(2,6-diazaspiro[3.3]heptan-2-ylmethyl)piperidin-1-yl)pyrimidin-2-yl)-5-methyl-6,7,8,9-tetrahydro-5H-pyrido[3′,4′:4,5]pyrrolo[2,3-c]pyridazin-3-yl)phenol. To a solution of 2,2,2-trifluoro-1-[6-[[1-[2-[12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-4-piperidyl]methyl]-2,6-diazaspiro[3.3]heptan-2-yl]ethanone (100 mg, 200 umol) in MeOH (1.4 mL) and H₂O (0.6 mL) was added K₂CO₃ (64.0 mg, 463 umol). The mixture was stirred at 25° C. for 5 hours. On completion, the reaction mixture was filtered and concentrated under reduced pressure to give a residue. The residue was purified by prep-HPLC (FA condition) to give the title compound (60 mg, 60% yield, FA) as an orange solid. LC-MS (ESI⁺) m/z 552.5 (M+H)⁺.

### Tert-butyl 4-ethynyl-2-hydroxybenzylcarbamate (Intermediate KP)

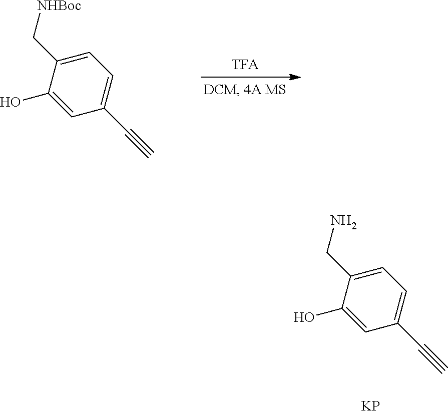 [SMILES: C#Cc1ccc(CNC(=O)OC(C)(C)C)c(O)c1 ; C#Cc1ccc(CN)c(O)c1 ; "KP"]

To a solution of tert-butyl N-[(4-ethynyl-2-hydroxy-phenyl)methyl]carbamate (2 g, 8.09 mmol, synthesized via Steps 1-3 of Intermediate HY) in DCM (20 mL) was added TFA (6.16 g, 54.02 mmol) and 4 Å molecular sieves (2 g, 8.09 mmol). The mixture was then stirred at 25° C. for 1 hr. On completion, the reaction mixture was concentrated under reduced pressure to give the title compound (1.18 g, 95% yield) as a white solid. LC-MS (ESI⁺) m/z 325.9 (M+H)⁺.

### (S)-1-(4-ethynyl-2-methoxyphenyl)ethanamine (Intermediate KQ)

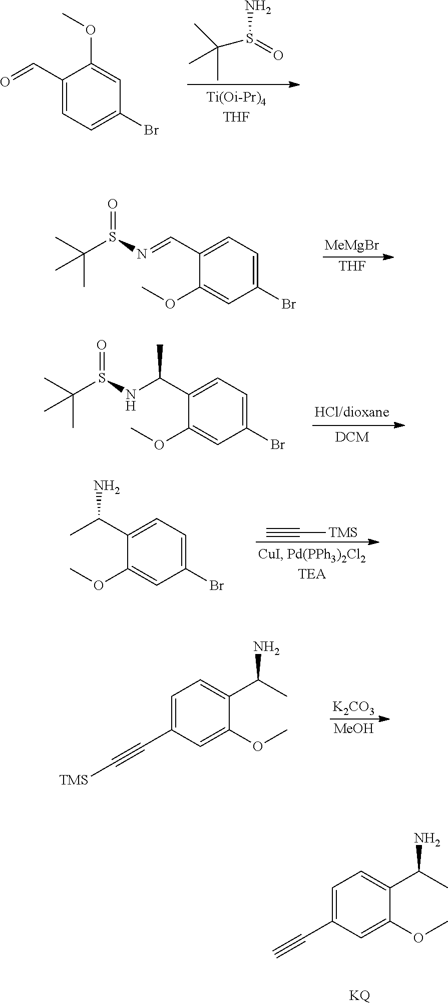

Step 1-(R,E)-N-(4-bromo-2-methoxybenzylidene)-2-methylpropane-2-sulfinamide. To a solution of 4-bromo-2-methoxy-benzaldehyde (25 g, 120 mmol) in THF (400 mL) was added Ti(Oi-Pr)₄ (82.6 g, 291 mmol), and (R)-2-methylpropane-2-sulfinamide (14.1 g, 116 mmol). The mixture was stirred at 65° C. for 12 hrs. On completion, the reaction mixture was concentrated under reduced pressure to remove solvent. The crude product was purified by column chromatography (SiO₂, Petroleum ether/Ethyl acetate=10/1 to 8/1) to give the title compound (32 g, 85% yield) as a yellow oily substance. ¹H NMR (400 MHz, DMSO-d₆) δ=8.78 (s, 1H), 7.79 (d, J=8.4 Hz, 1H), 7.39 (d, J=1.2 Hz, 1H), 7.24 (d, J=8.4 Hz, 1H), 3.92 (s, 3H), 1.26-1.06 (m, 9H); LC-MS (ESI⁺) m/z 319.9 (M+H⁺).

Step 2-(R)—N—((S)-1-(4-bromo-2-methoxyphenyl)ethyl)-2-methylpropane-2-sulfinamide. To a solution of (NE,R)—N-[(4-bromo-2-methoxy-phenyl)methylene]-2-methyl-propane-2-sulfinamide (5 g, 15.7 mmol) in THF (500 mL) was added MeMgBr (3 M, 61.5 mL) at −27° C. Then the mixture was stirred at −27° C. for 4 hours. On completion, the reaction mixture was partitioned between NH₄Cl (300 mL) and EA (400 mL). The organic layer was then concentrated under reduced pressure to give a residue. The residue was purified by column chromatography (SiO₂, Petroleum ether/ethyl acetate=5/1 to 3/1) to give the title compound (4.17 g, 79% yield) as a yellow solid. ¹H NMR (400 MHz, DMSO-d₆) δ=7.30 (d, J=7.6 Hz, 1H), 7.16-7.09 (m, 2H), 5.27 (d, J=6.0 Hz, 1H), 4.68 (quin, J=6.8 Hz, 1H), 3.81 (s, 3H), 1.37 (d, J=6.8 Hz, 3H), 1.09 (s, 9H); LC-MS (ESI⁺) m/z 335.9(M+H)⁺

Step 3-(S)-1-(4-bromo-2-methoxyphenyl)ethanamine. To a solution of (R)—N-[(1S)-1-(4-bromo-2-methoxy-phenyl)ethyl]-2-methyl-propane-2-sulfinamide (2 g, 6 mmol) in DCM (20 mL) was added HCl/dioxane (1 M, 5.98 mL) at 0° C. The mixture was stirred at 0° C.-25° C. for 2 hours. On completion, the reaction mixture was partitioned between solvent EA (30 mL) and H₂O (40 mL). The organic phase was washed with solvent NaCl_{(aq)} (10 mL×3), dried over NaSO₄, filtered and concentrated under reduced pressure to give a residue. The residue was purified by column chromatography (SiO₂, DCM: MeOH=50:1 to 20:1) to give the title compound (1.3 g, 81% yield, HCl) as a white solid. LC-MS (ESI⁺) m/z 214.9(M−NH₂+H⁺).

Step 4-(S)-1-(2-methoxy-4-((trimethylsilyl)ethynyl)phenyl)ethanamine. To a solution of ethynyl(trimethyl)silane (768 mg, 7.82 mmol, 1.08 mL) in TEA (3.5 mL) was added CuI (24.8 mg, 130 umol), (1S)-1-(4-bromo-2-methoxy-phenyl)ethanamine (300 mg, 1.30 mmol) and Pd(PPh₃)₂Cl₂ (45.8 mg, 65.2 umol). The mixture was degassed and purged with N₂ three times, and then the mixture was stirred at 80° C. for 12 h under N₂ atmosphere. On completion, the reaction mixture was partitioned between EA (20 mL) and H₂O (20 mL). The organic phase was separated, washed with NaCl (5 mL×3), dried over Na₂SO₄, filtered and concentrated under reduced pressure to give a residue. The residue was purified by column chromatography (SiO₂, DCM: MeOH=50:1 to 20:1) to give the title compound (270 mg, 76% yield) as a brown oily object. ¹H NMR (400 MHz, DMSO-d₆) δ=8.33 (s, 2H), 7.39 (d, J=8.2 Hz, 1H), 7.29-7.21 (m, 2H), 4.53 (s, 1H), 3.86 (s, 3H), 1.45 (d, J=6.8 Hz, 3H); LC-MS (ESI⁺) m/z231.2 (M−NH₂+H⁺).

Step 5-(S)-1-(4-ethynyl-2-methoxyphenyl)ethanamine. To a solution of (1S)-1-[2-methoxy-4-(2-trimethylsilylethynyl)phenyl]ethanamine (150 mg, 606 umol) in MeOH (3 mL) was added K₂CO₃ (167 mg, 1.21 mmol). The mixture was stirred at 25° C. for 1 hr. On completion, the reaction mixture was partitioned between EA (20 mL) and H₂O (10 mL). The organic phase was separated, washed with NaCl_{(aq)} (5 mL×3), dried over Na₂SO₄, filtered and concentrated under reduced pressure to give a residue. The residue was purified by column chromatography (SiO₂, DCM: MeOH=50:1) to give the title compound (30 mg, 27% yield) as a brown solid. LC-MS (ESI⁺) m/z159.2 (M−NH₂+H⁺).

### 2-(4-(2-((S)-2-(2-Hydroxyphenyl)-5,6,6a,7,9,10-hexahydro-8H-pyrazino[1′,2′:4,5]pyrazino[2,3-c]pyridazin-8-yl)pyrimidin-5-yl)piperidin-1-yl)spiro[3.5]nonane-7-carboxylic acid (Intermediate KR)

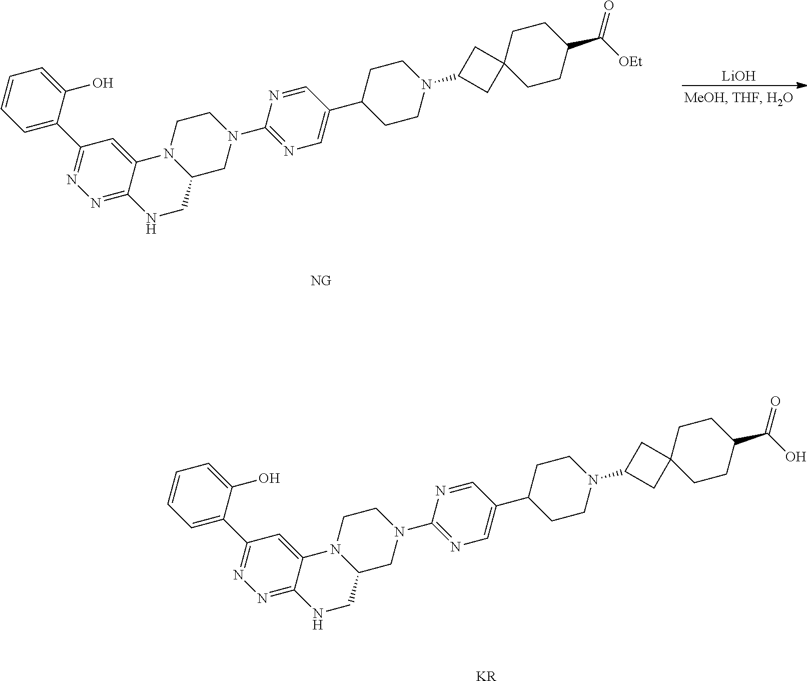

To a solution of ethyl 2-(4-(2-((S)-2-(2-hydroxyphenyl)-5,6,6a,7,9,10-hexahydro-8H-pyrazino[1′,2′:4,5]pyrazino[2,3-c]pyridazin-8-yl)pyrimidin-5-yl)piperidin-1-yl)spiro[3.5]nonane-7-carboxylate (1.00 g, 1.57 mmol, Intermediate NG) in MeOH (5 mL) THF (5 mL) H₂O (5 mL) was added LiOH·H₂O (657 mg, 15.7 mmol). The mixture was stirred at 25° C. for 12 hours. The mixture was filtered to give the solution The mixture was purified by reversed-phase Flash (0.1% HCl) to give the title compound (0.8 g, 98% purity, HCl salt) as a brown solid; LC-MS (ESI⁺) m/z 611.4 (M+H)⁺.

### 2-((tert-butoxycarbonyl)amino)-5-((tert-butyldimethylsilyl)oxy)-3,3-dimethylpentanoic acid (Intermediate KS)

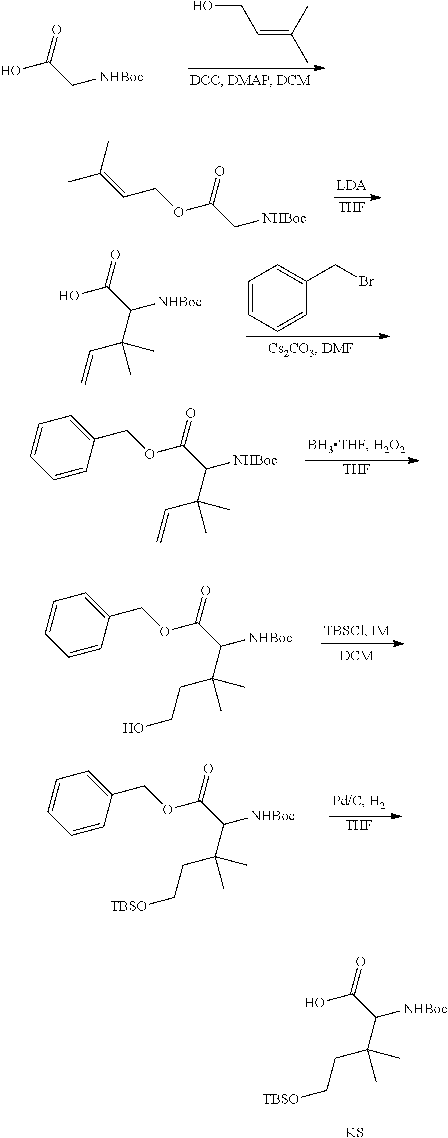

Step 1-3-Methylbut-2-en-1-yl 2-((tert-butoxycarbonyl)amino)acetate. To a solution of 2-(tert-butoxycarbonylamino)acetic acid (20 g, 114 mmol, CAS #4530-20-5) in DCM (300 mL) was added DMAP (2.32 g, 19.0 mmol) and DCC (29.4 g, 143 mmol, 28.9 mL) 3-methylbut-2-en-1-ol (8.19 g, 95.1 mmol, 9.52 mL, CAS #556-82-1) at −5° C. The mixture was stirred at 25° C. for 12 hours. On completion, the reaction mixture was quenched with sat. NH₄Cl (100 mL) at 25° C., and then diluted with EA (100 mL) and extracted with EA (300 mL×3). The combined organic layers were washed with sat. NaCl (300 mL×1), dried over Na₂SO₄, filtered and concentrated under reduced pressure to give a residue. The crude product was purification by MPLC (SiO₂, PE:EA=3/1 to 1:1) to give the title compound (23 g, 99% yield) as a white solid. ¹H NMR (400 MHz, DMSO-d₆) δ 5.35-5.28 (m, 1H), 5.09 (s, 1H), 4.61 (d, J=7.6 Hz, 2H), 3.87 (d, J=5.6 Hz, 2H), 1.71 (d, J=19.0 Hz, 6H), 1.42 (s, 9H).

Step 2-2-((Tert-butoxycarbonyl)amino)-3,3-dimethylpent-4-enoic acid. A mixture of 3-methylbut-2-enyl 2-(tert-butoxycarbonylamino)acetate (23.5 g, 96.6 mmol), and LDA (2 M, 121 mL) in THF (250 mL) at −65° C. was degassed and purged with N₂ three times. Then the mixture was stirred at 25° C. for 3 hours under N₂ atmosphere. On compeletion, to the mixture was add toluene (200 mL) at 25° C. and then add 10% H₂SO₄ (100 mL) until the pH=2-3. The organic layer was extracted by 10% NaOH 100 mL (100 mL×1). Then to the water layer was add 20% H₂SO₄ (100 mL) at −10° C. until the pH=2-3, then extracted by EA (300 mL). The organic layer was washed with sat. NaCl (150 mL), dried over Na₂SO₄, filtered and concentrated under reduced pressure to give the title compound (14 g) as a yellows oil. ¹H NMR (400 MHz, DMSO-d₆) δ 5.85 (dd, J=10.8, 17.2 Hz, 1H), 5.13-5.07 (m, 2H), 5.07-5.00 (m, 1H), 4.16 (d, J=9.2 Hz, 1H), 1.43 (s, 9H), 1.14 (d, J=2.8 Hz, 6H).

Step 3-Benzyl 2-((tert-butoxycarbonyl)amino)-3,3-dimethylpent-4-enoate. To a solution of 2-(tert-butoxycarbonylamino)-3,3-dimethyl-pent-4-enoic acid (7 g, 30 mmol) in DMF (150 mL) was added Cs₂CO₃ (9.37 g, 28.8 mmol) at 0° C. and the mixture was stirred 30 minutes. Then bromomethylbenzene (4.92 g, 28.8 mmol, 3.42 mL) was added at 0° C. The mixture was then stirred at 25° C. for 4 hrs. On completion, the reaction mixture was quenched with sat. NH₄Cl (50 mL) at 25° C., and then diluted with EA (5 mL) and extracted with EA (100 mL×3). The combined organic layers were washed with sat. NaCl (300 mL), dried over Na₂SO₄, filtered and concentrated under reduced pressure to give a residue. The crude product was purified by MPLC (SiO₂,PE:EA=1:0 to 100:1) to give the title compound (9 g, 94% yield) as a colorless oil. ¹H NMR (400 MHz, CHLOROFORM-d) δ 7.34-7.19 (m, 5H), 5.72 (dd, J=10.8, 17.6 Hz, 1H), 5.13-5.01 (m, 2H), 5.00-4.88 (m, 3H), 4.11 (d, J=9.6 Hz, 1H), 1.35 (s, 9H), 0.99 (d, J=7.2 Hz, 6H).

Step 4-Benzyl 2-((tert-butoxycarbonyl)amino)-5-hydroxy-3,3-dimethylpentanoate. To a 0° C. solution of 2,3-dimethylbut-2-ene (1 M, 17.5 mL) was added dropwise over 20 min of BH₃.THF (1 M, 17.5 mL). After the solution was stirred for an additional 60 minutes at 0° C., benzyl 2-(tert-butoxycarbonylamino)-3,3-dimethyl-pent-4-enoate (5 g, 15.0 mmol) in THF (8 mL) was added dropwise and the resulting solution was allowed to warm to ambient temperature over 1 hour. The solution was recooled to 0° C. and excess borane was quenched by the cautious addition of 1 mL of 1:1 THF/ethanol then H₂O₂(17.7 g, 156 mmol, 15.0 mL, 30% solution) was added. After the reaction mixture was stirred at ambient temperature for 14 hrs. The reaction mixture was quenched with sat. NaHSO₃ (20 mL) at 0° C., and then diluted with EA (10 mL) and extracted with EA (30 mL×3). The combined organic layers were washed with sat. NaCl (50 mL), dried over Na₂SO₄, filtered and concentrated under reduced pressure to give a residue. The crude product was purification by reversed-phase flash chromatography (0.1% FA condition) to give the title compound (1.4 g, 26% yield) as white solid. LC-MS (ESI⁺) m/z 252.2 (M−100+H)⁺.

Step 5-Benzyl 2-((tert-butoxycarbonyl)amino)-5-((tert-butyldimethylsilyl)oxy)-3,3-dimethylpentanoate. To a solution of benzyl 2-(tert-butoxycarbonylamino)-5-hydroxy-3,3-dimethyl-pentanoate (5.7 g, 16.2 mmol) in DCM (50 mL) was added imidazole (2.21 g, 32.4 mmol) and TBSCl (2.69 g, 17.8 mmol). The mixture was stirred at 25° C. for 12 hrs. The mixture was stirred at 25° C. for 12 hrs. On completion, the reaction mixture was quenched with H₂O (50 mL) at 25° C., and then diluted with DCM (20 mL) and extracted with DCM (50 mL×3). The combined organic layers were washed with sat. NaCl (50 mL) and dried over Na₂SO₄, filtered and concentrated under reduced pressure to give a residue. The crude product was purified by MPLC (SiO₂, PE:EA=5=30:1 to 10:1) to give the title compound (7 g, 81% yield) as a white solid. LC-MS (ESI⁺) m/z 366.3 (M−Boc+H)⁺.

Step 6-2-((Tert-butoxycarbonyl)amino)-5-((tert-butyldimethylsilyl)oxy)-3,3-dimethylpentanoic acid. To a solution of benzyl 2-(tert-butoxycarbonylamino)-5-[tert-butyl(dimethyl)silyl]oxy-3,3-dimethyl-pentanoate (6.5 g, 14 mmol) in MeOH (70 mL) was added Pd/C (650 mg, 10 wt %) and H₂ (15 psi). The mixture was stirred at 25° C. for 12 hrs. The mixture was filtered to remove the Pd/C and concentrated the solution under reduce pressure to give the title compound (5 g) as white solid. LC-MS (ESI⁺) m/z 376.4 (M+H)⁺.

### Tert-butyl ((S)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl)carbamate (Intermediate KT) & tert-butyl ((R)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl)carbamate (Intermediate KU)

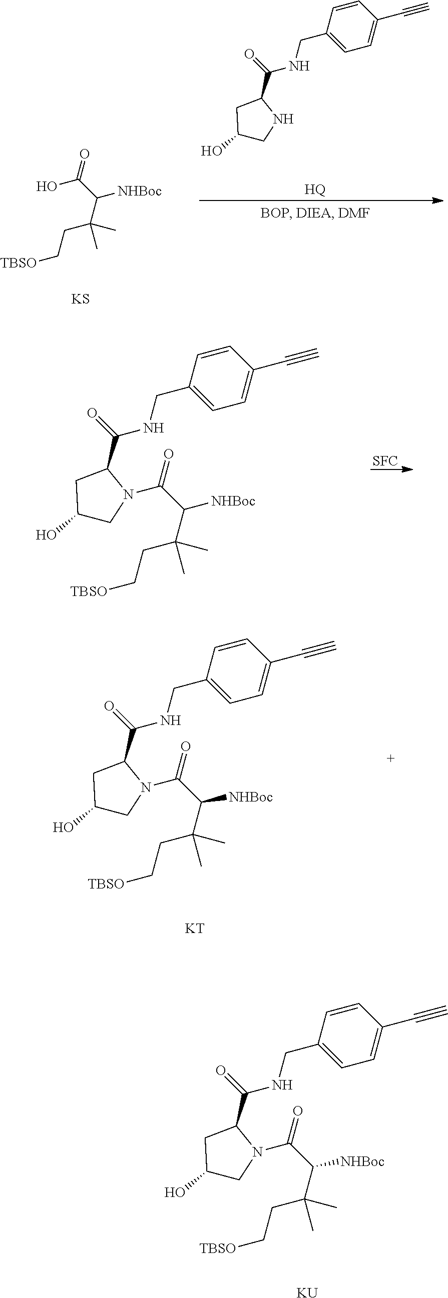

Step 1-Tert-butyl (5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl)carbamate. To a solution of (2S,4R)—N-[(4-ethynylphenyl)methyl]-4-hydroxy-pyrrolidine-2-carboxamide (715 mg, 2.93 mmol, Intermediate HQ) in DMF (15 mL) was added BOP (1.94 g, 4.39 mmol), DIEA (1.89 g, 14.6 mmol, 2.55 mL) and 2-(tert-butoxycarbonylamino)-5-[tert-butyl(dimethyl)silyl]oxy-3,3-dimethyl-pentanoic acid (2.2 g, 5.9 mmol, Intermediate KS). The mixture was stirred at 25° C. for 12 hrs. The mixture was filtered to give the solution. The mixture was purified by reversed-phase flash chromatography (0.1% NH₃·H₂O condition) to give the title compound (300 mg, 15% yield) as white solid. LC-MS (ESI⁺) m/z 602.4 (M+H)⁺.

Step 2-Tert-butyl ((S)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl)carbamate & tert-butyl ((R)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl)carbamate. Compound tert-butyl N-[4-[tert-butyl(dimethyl)silyl]oxy-1-[(2S,4R)-2-[(4-ethynylphenyl)methylcarbamoyl]-4-hydroxy-pyrrolidine-1-carbonyl]-2,2-dimethyl-butyl]carbamate (300 mg, 498 umol) was purified by SFC. The crude product was purified by SFC (column: REGIS(S,S)WHELK-01(250 mm×25 mm, 10 um); mobile phase: [0.1% NH₃H₂O ETOH]; B %: 30%-30%, 2; 20 min) to give tert-butyl ((S)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl)carbamate (140 mg, 43% yield) as white solid (LC-MS (ESI⁺) m/z 602.2 (M+H)⁺) and tert-butyl ((R)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl)carbamate (170 mg, 53% yield) as white solid (LC-MS (ESI⁺) m/z 602.2 (M+H)⁺).

### (2S,4R)-1-((S)-2-amino-5-hydroxy-3,3-dimethylpentanoyl)-N-(4-ethynylbenzyl)-4-hydroxypyrrolidine-2-carboxamide (Intermediate KV)

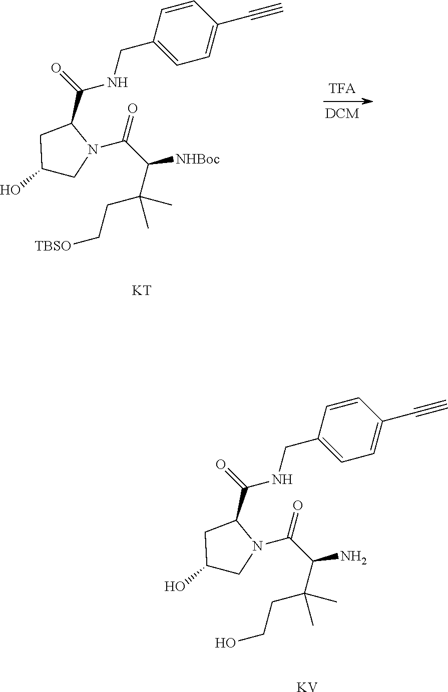 [SMILES: C#Cc1ccc(CNC(=O)[C@@H]2C[C@@H](O)CN2C(=O)[C@@H](NC(=O)OC(C)(C)C)C(C)(C)CCO[Si](C)(C)C(C)(C)C)cc1 ; "KT" ; C#Cc1ccc(CNC(=O)[C@@H]2C[C@@H](O)CN2C(=O)[C@@H](N)C(C)(C)CCO)cc1 ; "KV"]

To a solution of tert-butyl N-[(1S)-4-[tert-butyl(dimethyl)silyl]oxy-1-[(2S,4R)-2-[(4-ethynylphenyl)methylcarbamoyl]-4-hydroxy-pyrrolidine-1-carbonyl]-2,2-dimethyl-butyl]carbamate (100 mg, 200 umol, Intermediate KT) in DCM (2 mL) was added TFA (308 mg, 2.70 mmol, 200 uL) and 4 Å molecular sieves (50 mg, 200 umol). The mixture was stirred at 25° C. for 12 hrs. On completion, the mixture was filtered to remove the 4 Å molecular sieves and concentrated under reduce pressure to give a residue. The crude product was purificated by reversed phase flash chromatography (0.1% NH₃·H₂O) to give the title compound (50 mg, 72% yield) as white solid. LC-MS (ESI⁺) m/z 388.3 (M+H)⁺.

### (S)-tert-butyl (1-(4-ethynylphenyl)-3-hydroxypropyl)carbamate (Intermediate KW)

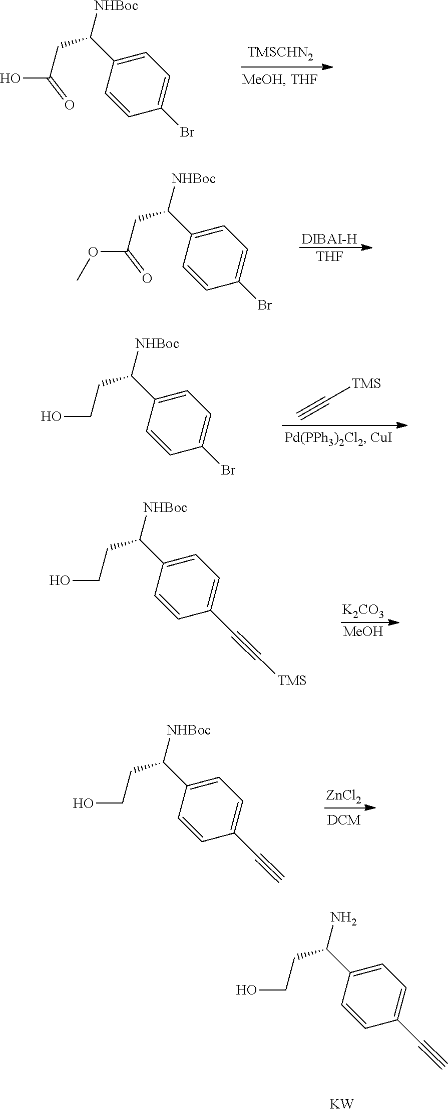

Step 1-(S)-3-(4-bromophenyl)-3-((tert-butoxycarbonyl)amino)propanoic acid. To a solution of (3S)-3-(4-bromophenyl)-3-(tert-butoxycarbonylamino)propanoic acid (10 g, 29.05 mmol, CAS #261165-06-4) in MeOH (30 mL) and THF (30 mL). Then TMSCHN₂ (2 M, 50.30 mL) was added until a persistent yellow solution was obtained. After 15 minutes of stirring, HOAc was added until the yellow color was quenched and gas evolution ceased. The mixture was stirred at 0° C. for 1 hr. On completion, the reaction was quenched with MeOH (50 ml) slowly at 0° C., and then the solution was stirred at 50° C. for another 12 hrs. On completion, glacial acetic acid (20 mL) was added to quench the reaction, then the mixture was filtered and concentrated under reduced pressure to give a residue. The residue was purified by column chromatography (SiO₂, Petroleum ether/Ethyl acetate=20/1 to 8/1) to give the title compound (7.24 g, 58% yield) as a white waxy solid. LC-MS (ESI⁺) m/z 380.1 (M+Na)⁺ and 243.0 (M−NHBoc)⁺.

Step 2-(S)-methyl 3-(4-bromophenyl)-3-((tert-butoxycarbonyl)amino)propanoate. To a solution of methyl (3S)-3-(4-bromophenyl)-3-(tert-butoxycarbonylamino)propanoate (1 g, 3 mmol) in THF (10 mL) was added DIBAL-H (1 M, 6.98 mL). The mixture was degassed and purged with N₂ three times, and then the mixture was stirred −78° C. for 0.5 hr under N₂ atmosphere. On completion, ethyl acetate (50 mL) was added to the mixture followed by saturated sodium potassium tartrate solution (20 mL) to quench the reaction. The reaction mixture was partitioned between water (50 mL) and EA (200 mL). The organic phase was separated, washed with brine (50 mL×3), dried over Na₂SO₄, filtered and concentrated under reduced pressure to give a residue. The residue was purified by column chromatography (SiO₂, Petroleum ether/Ethyl acetate=10/1 to 3/1) to give the title compound (450 mg, 46% yield) as a white waxy solid. LC-MS (ESI⁺) m/z 226.2 (M+H)⁺.

Step 3-(S)-tert-butyl (1-(4-bromophenyl)-3-hydroxypropyl)carbamate. To a solution of tert-butyl N-[(1S)-1-(4-bromophenyl)-3-hydroxy-propyl]carbamate (300 mg, 908.49 umol) and ethynyl(trimethyl)silane (892.30 mg, 9.08 mmol) in TEA (3 mL) was added Pd(PPh₃)₂Cl₂ (63.77 mg, 90.85 umol) and CuI (34.60 mg, 181.70 umol). The mixture was degassed and purged with N₂ three times, and then the mixture was stirred at 85° C. for 4 hr under N₂ atmosphere. On completion, the reaction mixture was partitioned between water (50 mL) and EA (150 mL). The organic phase was separated, washed with brine (50 mL×3), dried over Na₂SO₄, filtered and concentrated under reduced pressure to give a residue. The residue was purified by column chromatography (SiO₂, Petroleum ether/Ethyl acetate=6/1 to 1/1) to give the title compound (257 mg, 33% yield) as a yellow oil. LC-MS (ESI⁺) m z 370.1 (M+Na)⁺.

Step 4-(S)-tert-butyl (3-hydroxy-1-(4-((trimethylsilyl)ethynyl)phenyl)propyl)carbamate. To a solution of tert-butyl N-[(1S)-3-hydroxy-1-[4-(2-trimethylsilylethynyl)phenyl]propyl]carbamate (250 mg, 720 umol) in MeOH (2.5 mL) was added K₂CO₃ (198.85 mg, 1.44 mmol). The mixture was stirred at 25° C. for 2 hr. On completion, the reaction mixture was partitioned between water (40 mL) and EA (150 mL). The organic phase was separated, washed with brine (50 mL×3), dried over Na₂SO₄, filtered and concentrated under reduced pressure to give a residue. The residue was purified by column chromatography (SiO₂, Petroleum ether/Ethyl acetate=5/1 to 4/1) to give the title compound (66.7 mg, 33% yield) as a yellow oil.LC-MS (ESI⁺) m/z 298.1 (M+Na)⁺.

Step 5-(S)-tert-butyl (1-(4-ethynylphenyl)-3-hydroxypropyl)carbamate. To a solution of tert-butyl N-[(1S)-1-(4-ethynylphenyl)-3-hydroxy-propyl]carbamate (50 mg, 181.59 umol) in DCM (3 mL) was added 4 Å molecular sieves (50 mg). Then added TFA (462.00 mg, 4.05 mmol) was added and the mixture was stirred at 25° C. for 2 hr. On completion, the reaction mixture was concentrated under reduced pressure to remove solvent, filtered and concentrated under reduced pressure to give a residue. The crude product was purified by reversed-phase HPLC (0.1% NH3•H2O) to give the title compound (12.5 mg, 37% yield) as a yellow solid. LC-MS (ESI⁺) m/z 176.2 (M+H)⁺.

### 2-(6-(5-((3aR,6aS)-Hexahydropyrrolo[3,4-c]pyrrol-2(1H)-yl)pyrimidin-2-yl)-5-methyl-6,7,8,9-tetrahydro-5H-pyrido[3′,4′:4,5]pyrrolo[2,3-c]pyridazin-3-yl)phenol (Intermediate KX)

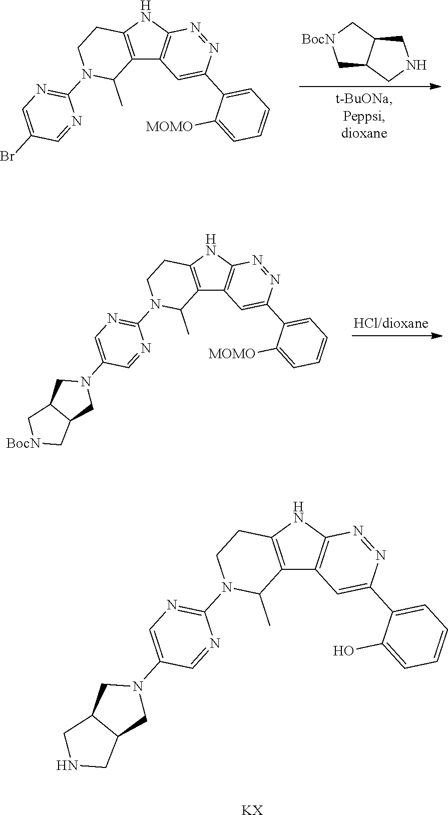

Step 1-(3aR,6aS)-tert-butyl 5-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)hexahydropyrrolo[3,4-c]pyrrole-2(1H)-carboxylate. To a solution of 4-(5-bromopyrimidin-2-yl)-12-[2-(methoxymethoxy)phenyl]-3-methyl-4,8,10,11-tetrazatricyclo[7.4.0.02,7]trideca-1(9),2(7),10,12-tetraene (1.5 g, 3.1 mmol, via Step 1 of Intermediate GQ) in dioxane (30 mL) was added tert-butyl(3aS,6aR)-2,3,3a,4,6,6a-hexahydro-1H-pyrrolo[3,4-c]pyrrole-5-carboxylate (1.32 g, 6.23 mmol, CAS #250275-15-1) and t-BuONa (2 M, 67 mL) and 1,3-bis[2,6-bis(1-propylbutyl)phenyl]-4,5-dichloro-2H-imidazol-1-ium-2-ide;3-chloropyridine;dichloropalladium (303 mg, 312 umol). The mixture was stirred at 100° C. for 12 hrs. On completion, the reaction mixture was partitioned between ethyl acetate (15×3 mL) and H₂O (5×3 mL). The organic phase was separated with brine (10 mL), dried over Na₂SO₄, filtered and concentrated under reduced pressure to give a residue. The residue was purified by column chromatography (SiO₂, Petroleum ether/Ethyl acetate=20/1 to 1/1) to give the title compound (1.09 g, 50% yield) as a yellow solid. LC-MS (ESI⁺) m/z 613.3 (M+H)⁺.

Step 2-2-(6-(5-((3aR,6aS)-hexahydropyrrolo[3,4-c]pyrrol-2(1H)-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl (3aS,6aR)-2-[2-[12-[2-(methoxymethoxy)phenyl]-3-methyl-4,8,10,11-tetrazatricyclo[7.4.0.02,7]trideca-1(9),2(7),10,12-tetraen-4-yl]pyrimidin-5-yl]-1,3,3a,4,6,6a-hexahydropyrrolo[3,4-c]pyrrole-5-carboxylate (1.09 g, 1.78 mmol) was added HCl/dioxane (8 M, 13.3 mL). The mixture was stirred at 25° C. for 2 hrs. On completion, the reaction mixture concentrated under reduced pressure to give a residue. The residue was purified by HPLC (FA) to give the title compound (600 mg, 63% yield) as a red solid. LC-MS (ESI⁺) m/z 469.2 (M+H)⁺.

### 2-(6-(5-((3aR,6aS)-5-(2-Azaspiro[3.3]heptan-6-yl)hexahydropyrrolo[3,4-c]pyrrol-2(1H)-yl)pyrimidin-2-yl)-5-methyl-6,7,8,9-tetrahydro-5H-pyrido[3′,4′:4,5]pyrrolo[2,3-c]pyridazin-3-yl)phenol (Intermediate KY)

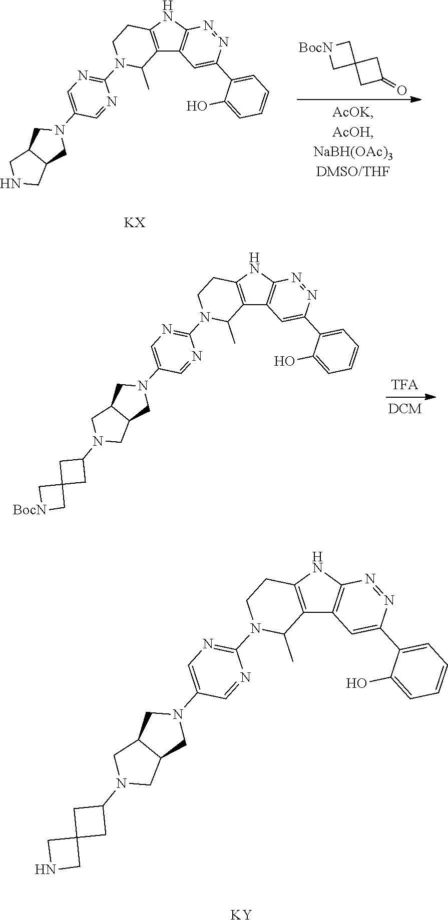

Step 1-Tert-butyl 6-((3aR,6aS)-5-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)hexahydropyrrolo[3,4-c]pyrrol-2(1H)-yl)-2-azaspiro[3.3]heptane-2-carboxylate. To a solution of 2-[4-[5-[(3aR,6aS)-2,3,3a,4,6,6a-hexahydro-1H-pyrrolo[3,4-c]pyrrol-5-yl]pyrimidin-2-yl]-3-methyl-4,8,10,11-tetrazatricyclo[7.4.0.02,7]trideca-1(9),2(7),10,12-tetraen-12-yl]phenol (200 mg, 400 umol, Intermediate KX) in DMSO (2 mL) and THF (10 mL) added AcOK (126 mg, 1.28 mmol). After the addition, the mixture was stirred at 0° C. for 0.5 hour, and then tert-butyl6-oxo-2-azaspiro[3.3]heptane-2-carboxylate (117 mg, 555 umol) and NaBH(OAc)₃ (271 mg, 1.28 mmol) was added and at 0° C. for 1.5 hours. Finally, AcOH (76.9 mg, 1.28 mmol, 73.2 uL) was added at 0° C. and the resulting mixture was stirring at 0° C. for 12 hrs. On completion, the reaction mixture was quenched with water (0.5 mL). The residue was purified by HPLC (FA condition) to give the title compound (150 mg, 48% yield) as a yellow solid. LC-MS (ESI⁺) m/z 664.5 (M+H)⁺.

Step 2-2-(6-(5-((3aR,6aS)-5-(2-azaspiro[3.3]heptan-6-yl)hexahydropyrrolo[3,4-c]pyrrol-2(1H)-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 6-[(3aR,6aS)-2-[2-[12-(2-hydroxyphenyl)-3-methyl-4,8,10,11-tetrazatricyclo[7.4.0.02,7]trideca-1(9),2(7),10,12-tetraen-4-yl]pyrimidin-5-yl]-1,3,3a,4,6,6a-hexahydropyrrolo[3,4-c]pyrrol-5-yl]-2-azaspiro[3.3]heptane-2-carboxylate (120 mg, 180 umol) was added DCM (4 mL) and TFA (1.28 g, 11.2 mmol, 831 uL). The mixture was stirred at 25° C. for 2 hrs. On completion, the reaction mixture was concentrated under reduced pressure to remove DCM and then dry with N₂ to remove TFA. The residue was purified by prep-HPLC (FA condition) to give the title compound (100 mg, 94% yield) as a yellow solid. LC-MS (ESI⁺) m/z 564.2 (M+H)⁺.

### (2S,4R)-4-((tert-butyldimethylsilyl)oxy)-N—((S)-1-(4-ethynylphenyl)ethyl)-1-((S)-2-hydroxy-3,3-dimethylbutanoyl)pyrrolidine-2-carboxamide (Intermediate KZ)

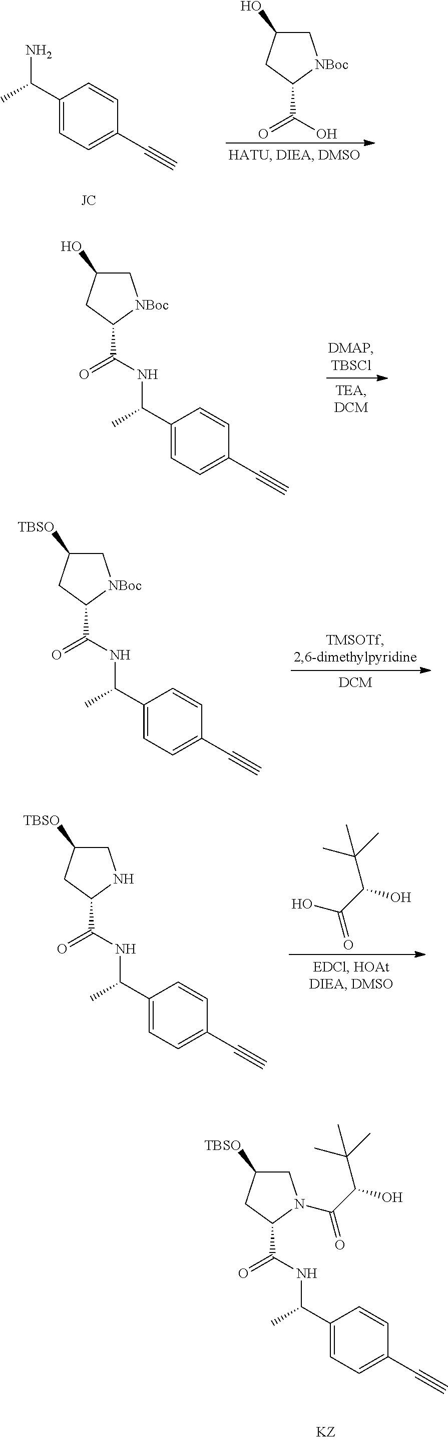

Step 1-(2S,4R)-tert-butyl 2-(((S)-1-(4-ethynylphenyl)ethyl)carbamoyl)-4-hydroxypyrrolidine-1-carboxylate. To a solution of (2S,4R)-1-tert-butoxycarbonyl-4-hydroxy-pyrrolidine-2-carboxylic acid (1.45 g, 6.28 mmol, CAS #13726-69-7) and (1S)-1-(4-ethynylphenyl)ethanamine (800 mg, 4.18 mmol, Intermediate JC) in DMSO (8 mL) was added HATU (2.39 g, 6.28 mmol) and DIEA (1.62 g, 12.5 mmol). The mixture was then stirred at 25° C. for 2 hrs. On completion, the reaction mixture was partitioned between H₂O (20 mL) and ethyl acetate (60 mL). The organic phase was separated, washed with brine (20 mL×5), dried over anhydrous sodium sulfate, filtered and concentrated under reduced pressure to give a residue. The residue was purified by column chromatography (SiO₂, Dichloromethane/Methanol=100/1 to 30/1) to give the title compound (1.68 g, 95% yield) as a yellow oil. LC-MS (ESI⁺) m/z 259.1 (M+H)⁺.

Step 2-(2S,4R)-tert-butyl 4-((tert-butyldimethylsilyl)oxy)-2-(((S)-1-(4-ethynylphenyl)ethyl)carbamoyl)pyrrolidine-1-carboxylate. To a solution of tert-butyl (2S, 4R)-2-[[(1S)-1-(4-ethynylphenyl)ethyl]carbamoyl]-4-hydroxy-pyrrolidine-1-carboxylate (4.68 g, 13.0 mmol) in DCM (50 mL) was added TEA (3.96 g, 39.1 mmol), DMAP (638 mg, 5.22 mmol) and TBSCl (3.94 g, 26.1 mmol) at 0° C. The mixture was stirred at 25° C. for 12 hrs. On completion, the reaction mixture was partitioned between H₂O (50 mL) and dichloromethane (100 mL). The organic phase was separated, dried over anhydrous sodium sulfate, filtered and concentrated under reduced pressure to give a residue. The residue was purified by column chromatography (SiO₂, Petroleum ether/Ethyl acetate=20/1 to 10/1) to give the title compound (3.04 g, 44% yield) as a white solid. LC-MS (ESI⁺) m/z 473.3 (M+H)⁺.

Step 3-(2S,4R)-4-((tert-butyldimethylsilyl)oxy)-N—((S)-1-(4-ethynylphenyl)ethyl)pyrrolidine-2-carboxamide. To a solution of tert-butyl (2S, 4R)-4-[tert-butyl(dimethyl)silyl]oxy-2-[[(1S)-1-(4-ethynylphenyl)ethyl]carbamoyl]pyrrolidine-1-carboxylate (1.5 g, 3.2 mmol) in DCM (15 mL) was added TMSOTf (2.11 g, 9.51 mmol) and 2,6-dimethylpyridine (1.71 g, 15.8 mmol). The mixture was stirred at 25° C. for 1 hr. On completion, the reaction mixture was concentrated under reduced pressure to give a residue. The residue was purified by reversed-phase HPLC (0.1% NH₃•H₂O) to give the title compound (0.945 g, 79% yield) as a yellow oil. LC-MS (ESI⁺) m/z 373.6 (M+H)⁺.

Step 4-(2S,4R)-4-((tert-butyldimethylsilyl)oxy)-N—((S)-1-(4-ethynylphenyl)ethyl)-1-((S)-2-hydroxy-3,3-dimethylbutanoyl)pyrrolidine-2-carboxamide. To a solution of (2S,4R)-4-[tert-butyl(dimethyl)silyl]oxy-N-[(1S)-1-(4-ethynylphenyl)ethyl]pyrrolidine-2-carboxamide (280 mg, 751 umol) in DMSO (3 mL) was added DIEA (291 mg, 2.25 mmol), EDCI (360 mg, 1.88 mmol), HOAt (255 mg, 1.88 mmol) and (2S)-2-hydroxy-3,3-dimethyl-butanoic acid (109 mg, 826 umol, CAS #21641-92-9). The mixture was then stirred at 25° C. for 4 hours. On completion, the reaction mixture was partitioned between H₂O (15 mL) and ethyl acetate (30 mL). The organic phase was separated, washed with brine (10 mL×3), dried over anhydrous sodium sulfate, filtered and concentrated under reduced pressure to give a residue. The residue was purified by column chromatography (SiO₂, Petroleum ether/Ethyl acetate=20/1 to 8/1) to give the title compound (210 mg, 55% yield) as a yellow oil. ¹H NMR (400 MHz, DMSO-d₆) δ=8.42 (d, J=7.6 Hz, 1H), 7.40 (d, J=8.4 Hz, 2H), 7.29-7.25 (m, 2H), 4.85 (quin, J=7.2 Hz, 1H), 4.51-4.45 (m, 2H), 4.42 (s, 1H), 4.20 (d, J=8.4 Hz, 1H), 4.07-3.97 (m, 1H), 3.88 (d, J=8.0 Hz, 1H), 3.63-3.58 (m, 1H), 3.53-3.48 (m, 1H), 2.52 (d, J=2.0 Hz, 1H), 2.00 (s, 1H), 1.99 (s, 1H), 1.74 (ddd, J=4.4, 8.8, 12.8 Hz, 1H), 1.33 (d, J=7.2 Hz, 3H), 1.01-0.96 (m, 1H), 0.90 (s, 9H), 0.85 (s, 9H), 0.07-0.04 (m, 6H). LC-MS (ESI⁺) m/z 487.8 (M+H)⁺.

### (S)-1-((2S,4R)-4-((tert-butyldimethylsilyl)oxy)-2-(((S)-1-(4-ethynylphenyl)ethyl)carbamoyl)pyrrolidin-1-yl)-3,3-dimethyl-1-oxobutan-2-yl phenyl carbonate (Intermediate LA)

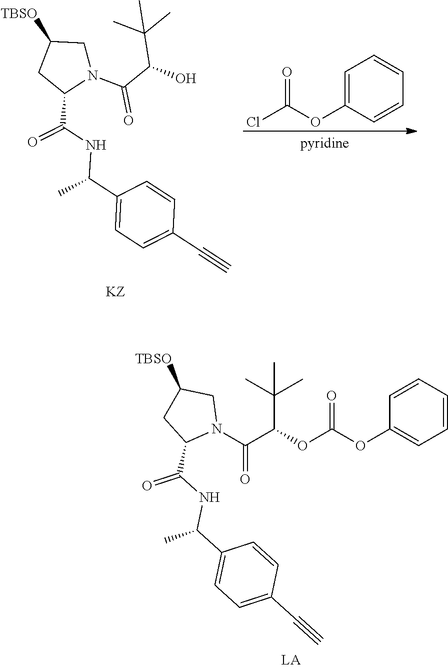 [SMILES: C#Cc1ccc([C@H](C)NC(=O)[C@@H]2C[C@@H](O[Si](C)(C)C(C)(C)C)CN2C(=O)[C@@H](O)C(C)(C)C)cc1 ; O=C(Cl)Oc1ccccc1 ; C#Cc1ccc([C@H](C)NC(=O)[C@@H]2C[C@@H](O[Si](C)(C)C(C)(C)C)CN2C(=O)[C@@H](OC(=O)Oc2ccccc2)C(C)(C)C)cc1]

To a solution of (2S,4R)-4-[tert-butyl(dimethyl)silyl]oxy-N-[(1S)-1-(4-ethynylphenyl)ethyl]-1-[(2S)-2-hydroxy-3,3-dimethyl-butanoyl]pyrrolidine-2-carboxamide (200 mg, 410 umol, Intermediate KZ) in pyridine (2 mL) was added phenyl carbonochloridate (128 mg, 821 umol, CAS #1885-14-9) at 0° C. The mixture was stirred at 0-25° C. for 3 hrs. On completion, the reaction mixture was quenched with MeOH (1 mL) at 0° C., filtered and concentrated under reduced pressure to give a residue. The crude product was purified by reversed-phase HPLC (0.1% FA condition) to give the title compound (130 mg, 47% yield) as a white solid. LC-MS (ESI⁺) m/z 607.7 (M+H)⁺.

### Phenyl (S)-2-(8-(5-(4-(piperazin-1-ylmethyl)piperidin-1-yl)pyrimidin-2-yl)-6,6a,7,8,9,10-hexahydro-5H-pyrazino[1′,2′:4,5]pyrazino[2,3-c]pyridazin-2-yl)phenol (Intermediate LB)

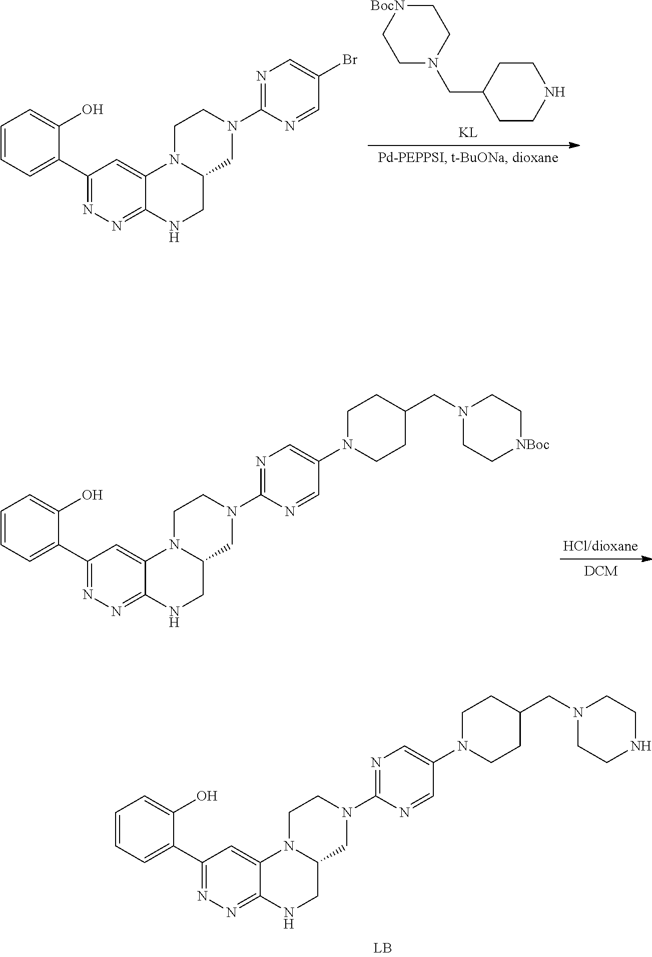

Step 1-(S)-tert-butyl 4-((1-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-4-yl)methyl)piperazine-1-carboxylate. To a mixture of (S)-2-(8-(5-bromopyrimidin-2-yl)-6,6a,7,8,9,10-hexahydro-5H-pyrazino[1′,2′:4,5]pyrazino[2,3-c]pyridazin-2-yl)phenol (500 mg, 1.14 mmol, synthesized via Step 1 of Intermediate EA) and tert-butyl 4-(piperidin-4-ylmethyl)piperazine-1-carboxylate (483 mg, 1.70 mmol, Intermediate KL) in dioxane (5 mL) was added tBuONa (2 M, 1.42 mL) and PD-PEPPSI™-IPENT catalyst (90.1 mg, 114 umol). The mixture was degassed and purged with N₂ three times. Then the mixture was stirred at 100° C. for 2 hrs. On completion, the reaction mixture was diluted with H₂O (50 mL) and extracted with DCM (400 mL×4). The combined organic layers were dried over Na₂SO₄, filtered and concentrated under reduced pressure to give a residue. The crude product was purified by prep-HPLC (0.1% FA condition) to give the title compound (100 mg, 8% yield, FA) as a red solid. LC-MS (ESI⁺) m/z 643.5 (M+H)⁺.

Step 2-Phenyl (S)-2-(8-(5-(4-(piperazin-1-ylmethyl)piperidin-1-yl)pyrimidin-2-yl)-6,6a,7,8,9,10-hexahydro-5H-pyrazino[1′,2′:4,5]pyrazino[2,3-c]pyridazin-2-yl)phenol. To a solution of (S)-tert-butyl 4-((1-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-4-yl)methyl)piperazine-1-carboxylate (100 mg, 155 umol) in DCM (4 mL) was added HCl/dioxane (9 M, 0.4 mL). The mixture was stirred at 25° C. for 30 mins. On completion, the reaction mixture was concentrated in vacuo to give a residue. The residue was purified by prep-HPLC (0.1% HCl) to give the title compound (55 mg, 58% yield) as a rose red solid. LC-MS (ESI⁺) m/z 543.4 (M+H)⁺.

### 2-(6-(5-(2,7-diazaspiro[4.4]nonan-2-yl)pyrimidin-2-yl)-5-methyl-6,7,8,9-tetrahydro-5H-pyrido[3′,4′:4,5]pyrrolo[2,3-c]pyridazin-3-yl)phenol (Intermediate LC)

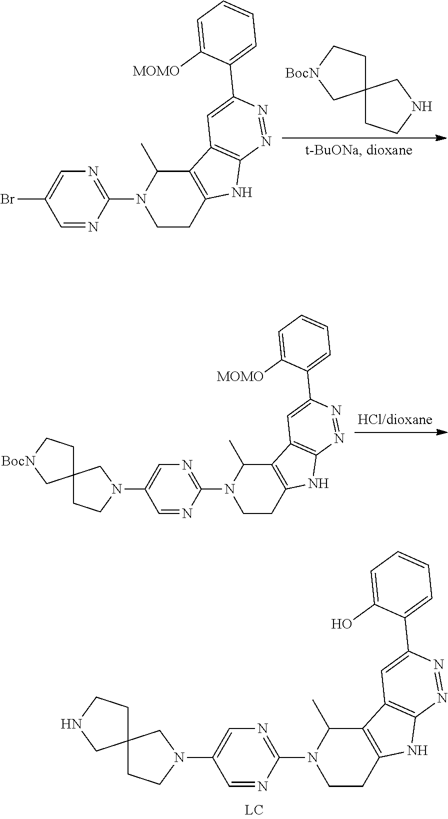

Step 1-Tert-butyl 7-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)-2,7-diazaspiro[4.4]nonane-2-carboxylate. To a solution of 4-(5-bromopyrimidin-2-yl)-12-[2-(methoxymethoxy)phenyl]-3-methyl-4,8,10,11-tetrazatricyclo[7.4.0.02,7]trideca-1(9),2(7),10,12-tetraene (300 mg, 623 umol, synthesized via Step 1 of Intermediate GQ) in dioxane (15 mL) was added tBuONa (179 mg, 1.87 mmol), tert-butyl 2,7-diazaspiro[4.4]nonane-2-carboxylate (211 mg, 934 umol, CAS #236406-49-8) and 1,3-bis[2,6-bis(1-propylbutyl)phenyl]-4,5-dichloro-2H-imidazol-1-ium-2-ide;3-chloropyridine;dichloropalladium (60.6 mg, 62.3 umol). The mixture was stirred at 100° C. for 12 hours. On completion, the reaction mixture was extracted with DCM 90 mL (30 mL×3). The combined organic layers were washed with aqueous NaCl (30 mL×3), dried over Na₂SO₄ and concentrated in vacuo. The residue was purified by prep-HPLC (FA) to give the title compound (701 mg, 41% yield) as a yellow solid. LC-MS (ESI⁺) m/z 627.4 (M+H)⁺.

Step 2-2-(6-(5-(2,7-Diazaspiro[4.4]nonan-2-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 7-[2-[12-[2-(methoxymethoxy)phenyl]-3-methyl-4,8,10,11-tetrazatricyclo[7.4.0.02,7]trideca-1(9),2(7),10,12-tetraen-4-yl]pyrimidin-5-yl]-2,7-diazaspiro[4.4]nonane-2-carboxylate (511 mg, 815 umol) in HCl/dioxane (9 mL). The mixture was stirred at 25° C. for 2 hours. On completion, the reaction mixture was concentrated under reduced pressure to give the title compound (701 mg) as a yellow solid. LC-MS (ESI⁺) m/z 482.9 (M+H)⁺.

### 2-(6-(5-(7-(2-azaspiro[3.3]heptan-6-yl)-2,7-diazaspiro[4.4]nonan-2-yl)pyrimidin-2-yl)-5-methyl-6,7,8,9-tetrahydro-5H-pyrido[3′,4′:4,5]pyrrolo[2,3-c]pyridazin-3-yl)phenol (Intermediate LD)

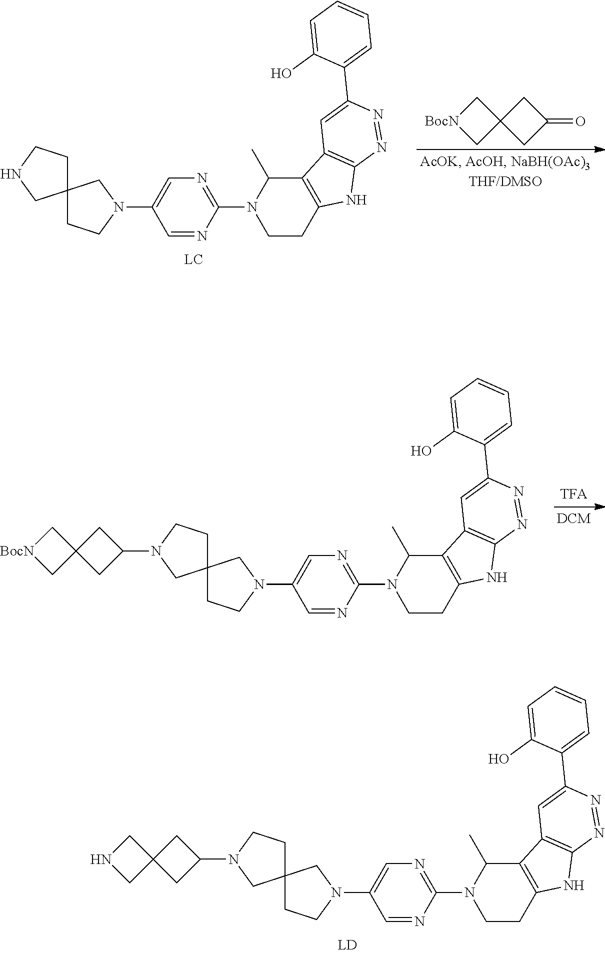 [SMILES: CC1c2c([nH]c3nnc(-c4ccccc4O)cc23)CCN1c1ncc(N2CCC3(CCNC3)C2)cn1 ; CC(C)(C)OC(=O)N1CC2(CC(=O)C2)C1 ; CC1c2c([nH]c3nnc(-c4ccccc4O)cc23)CCN1c1ncc(N2CCC3(CCN(C4CC5(C4)CN(C(=O)OC(C)(C)C)C5)C3)C2)cn1 ; CC1c2c([nH]c3nnc(-c4ccccc4O)cc23)CCN1c1ncc(N2CCC3(CCN(C4CC5(CNC5)C4)C3)C2)cn1]

Step 1-Tert-butyl 6-(7-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)-2,7-diazaspiro[4.4]nonan-2-yl)-2-azaspiro[3.3]heptane-2-carboxylate. To a solution of 2-[4-[5-(2,7-diazaspiro[4.4]nonan-2-yl)pyrimidin-2-yl]-3-methyl-4,8,10,11-tetrazatricyclo[7.4.0.02,7]trideca-1(9),2(7),10,12-tetraen-12-yl]phenol (701 mg, 1.45 mmol, Intermediate LC) in THF (10 mL), DMSO (10 mL) was added AcOK (426 mg, 4.35 mmol) at 25° C. for 0.5 hours. Next, AcOH (261 mg, 4.35 mmol, 248 uL) and tert-butyl 6-oxo-2-azaspiro[3.3]heptane-2-carboxylate (367 mg, 1.74 mmol, CAS #1147557-97-8) were added to the mixture at 25° C. and the mixture was stirred for 1.5 hours. At last, to the solution was added NaBH(OAc)₃ (921 mg, 4.35 mmol) at 0° C. Then the mixture was stirred at 25° C. for 12 hrs. On completion, the reaction was added water (2 mL), and then concentrated under reduced pressure to give the residue. The residue was purified by prep-HPLC (FA) to give the title compound (450 mg, 42% yield) as a yellow solid. LC-MS (ESI⁺) m/z 678.5 (M+H)⁺.

Step 2-2-(6-(5-(7-(2-azaspiro[3.3]heptan-6-yl)-2,7-diazaspiro[4.4]nonan-2-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 6-[7-[2-[12-(2-hydroxyphenyl)-3-methyl-4,8,10,11-tetrazatricyclo[7.4.0.02,7]trideca-1(9),2(7),10,12-tetraen-4-yl]pyrimidin-5-yl]-2,7-diazaspiro[4.4]nonan-2-yl]-2-azaspiro[3.3]heptane-2-carboxylate (410 mg, 604 umol) in DCM (10 mL) was added TFA (3.08 g, 27.0 mmol, 2 mL). The mixture was stirred at 25° C. for 2 hours. On completion, the reaction mixture was concentrated under reduced pressure to give the title compound (650 mg) as a yellow solid. LC-MS (ESI⁺) m/z 578.3 (M+H)⁺.

### (R)-3-(2-(methoxymethoxy)phenyl)-5-methyl-6,7,8,9-tetrahydro-5H-pyrido[3′,4′:4,5]pyrrolo[2,3-c]pyridazine (Intermediate LE)

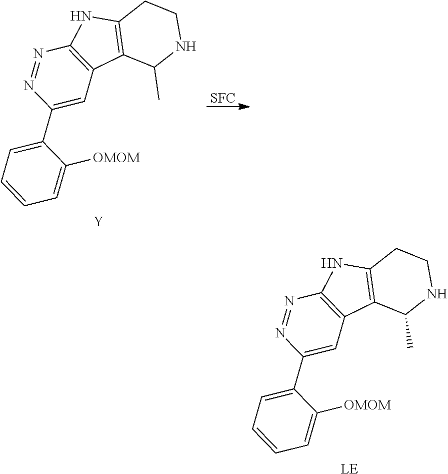

3-(2-(methoxymethoxy)phenyl)-5-methyl-6,7,8,9-tetrahydro-5H-pyrido[3′,4′:4,5]pyrrolo[2,3-c]pyridazine (Intermediate Y) was purified by SFC (column: DAICEL CHIRALPAK AS (250 mm×30 mm, 10 um); mobile phase: [0.1% NH₃H₂O EtOH]; B %: 40%-40%, 4; 95 min). to give (3S)-12-[2-(methoxymethoxy)phenyl]-3-methyl-4,8,10,11-tetrazatricyclo[7.4.0.0^{2,7}]trideca-1(9),2(7),10,12-tetraene (1 g, 49% yield) was obtained as a white solid. LC-MS (ESI⁺) m/z 325.1 (M+H)⁺.

### (R)-2-(5-methyl-6-(piperidin-4-yl)-6,7,8,9-tetrahydro-5H-pyrido[3′,4′:4,5]pyrrolo[2,3-c]pyridazin-3-yl)phenol (Intermediate LF)

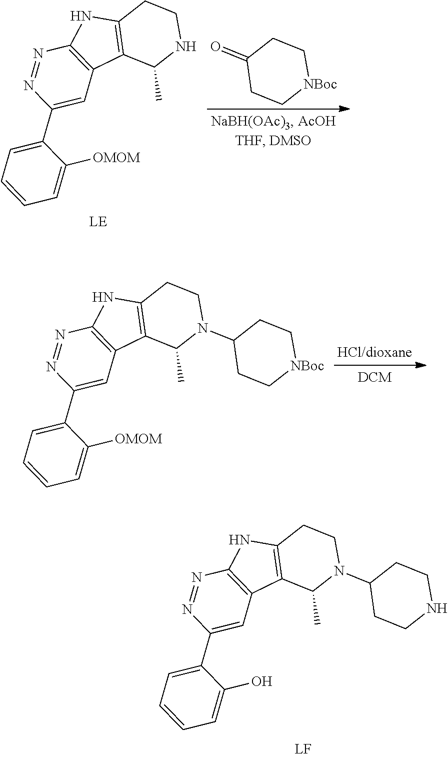

Step 1-(R)-tert-butyl 4-(3-(2-(methoxymethoxy)phenyl)-5-methyl-7,8-dihydro-5H-pyrido[3′,4′:4,5]pyrrolo[2,3-c]pyridazin-6(9H)-yl)piperidine-1-carboxylate. A mixture of (3R)-12-[2-(methoxymethoxy)phenyl]-3-methyl-4,8,10,11-tetrazatricyclo[7.4.0.0^{2,7}]trideca-1(9),2(7),10,12-tetraene (770 mg, 2.37 mmol, Intermediate LE), tert-butyl 4-oxopiperidine-1-carboxylate (1.18 g, 5.93 mmol, CAS #79099-07-3), AcOH (143 mg, 2.37 mmol), 4 Å molecular sieves (2 g, 2.37 mmol) and NaBH(OAc)₃ (2.01 g, 9.50 mmol) in THF (7 mL) and DMSO (7 mL) was degassed and purged with N₂ three times. Then the mixture was stirred at 25° C. for 12 hours under N₂ atmosphere. The crude product was purified by reversed-phase HPLC (0.1% FA condition) to give the title compound (700 mg, 51% yield, FA) as a red solid. LC-MS (ESI⁺) m/z 508.3 (M+H)⁺.

Step 2-(R)-2-(5-methyl-6-(piperidin-4-yl)-6,7,8,9-tetrahydro-5H-pyrido[3′,4′:4,5]pyrrolo[2,3-c]pyridazin-3-yl)phenol. To a solution of tert-butyl 4-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]piperidine-1-carboxylate (650 mg, 1.28 mmol) in DCM (12 mL) was added HCl/dioxane (2 M, 4.55 mL). The mixture was stirred at 25° C. for 2 hrs. On completion, the reaction mixture was concentrated under reduced pressure to give the title compound (900 mg, HCl) as a yellow solid. LC-MS (ESI⁺) m z 364.2 (M+H)⁺.

### (R)-2-(6-(1-(2-azaspiro[3.3]heptan-6-yl)piperidin-4-yl)-5-methyl-6,7,8,9-tetrahydro-5H-pyrido[3′,4′:4,5]pyrrolo[2,3-c]pyridazin-3-yl)phenol (Intermediate LG)

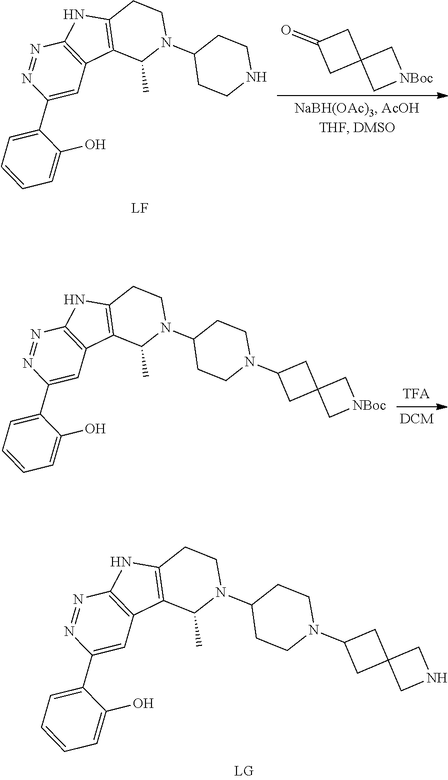 [SMILES: C[C@@H]1c2c([nH]c3nnc(-c4ccccc4O)cc23)CCN1C1CCNCC1 ; CC(C)(C)OC(=O)N1CC2(CC(=O)C2)C1 ; C[C@@H]1c2c([nH]c3nnc(-c4ccccc4O)cc23)CCN1C1CCN(C2CC3(C2)CN(C(=O)OC(C)(C)C)C3)CC1 ; C[C@@H]1c2c([nH]c3nnc(-c4ccccc4O)cc23)CCN1C1CCN(C2CC3(CNC3)C2)CC1]

Step 1-(R)-tert-butyl 6-(4-(3-(2-hydroxyphenyl)-5-methyl-7,8-dihydro-5H-pyrido[3′,4′:4,5]pyrrolo[2,3-c]pyridazin-6(9H)-yl)piperidin-1-yl)-2-azaspiro[3.3]heptane-2-carboxylate. A mixture of 2-[(3R)-3-methyl-4-(4-piperidyl)-4,8,10,11-tetrazatricyclo[7.4.0.0^{2,7}]trideca-1(9),2(7),10,12-tetraen-12-yl]phenol (900 mg, 2 mmol, HCl, Intermediate LF), tert-butyl 6-oxo-2-azaspiro[3.3]heptane-2-carboxylate (571 mg, 2.70 mmol, CAS #1147557-97-8), KOAc (552 mg, 5.63 mmol), HOAc (338 mg, 5.63 mmol) and NaBH(OAc)₃ (1.43 g, 6.75 mmol) in THF (4 mL) and DMSO (9 mL) was degassed and purged with N₂ three times. Then the mixture was stirred at 25° C. for 12 hours under N₂ atmosphere. On completion, the reaction mixture was quenched by addition of sat. aq. Na₂CO₃ (20 mL) at 25° C., and then diluted with DCM (20 mL) and extracted with DCM (20 mL×3). The combined organic layers were dried over Na₂SO₄, filtered and concentrated under reduced pressure to give a residue. The residue was purified by column chromatography (SiO2, DCM: MeOH=50/1 to 10/1) to give the title compound (900 mg, 67% yield) as a yellow solid. LC-MS (ESI⁺) m/z 559.2 (M+H)⁺.

Step 2-(R)-2-(6-(1-(2-azaspiro[3.3]heptan-6-yl)piperidin-4-yl)-5-methyl-6,7,8,9-tetrahydro-5H-pyrido[3′,4′:4,5]pyrrolo[2,3-c]pyridazin-3-yl)phenol. To a solution of tert-butyl 6-[4-[(3R)-12-(2-hydroxyphenyl)-3-methyl-4,8,10,11-tetrazatricyclo[7.4.0.0^{2,7}]trideca-1(9),2(7),10,12-tetraen-4-yl]-1-piperidyl]-2-azaspiro[3.3]heptane-2-carboxylate (800 mg, 1.43 mmol) in DCM (10 mL) was added TFA (1.54 g, 13.5 mmol) and 4 Å molecular sieves (800 mg, 1.43 mmol). The mixture was stirred at 25° C. for 2 hours. On completion, the crude product was purified by reversed-phase HPLC (0.1% FA condition) to give the title compound (650 mg, 89% yield, FA) as a white solid. LC-MS (ESI⁺) m z 459.2 (M+H)⁺.

### 2-(5-Methyl-6-(piperidin-4-yl)-6,7,8,9-tetrahydro-5H-pyrido[3′,4′:4,5]pyrrolo[2,3-c]pyridazin-3-yl)phenol (Intermediate LH)

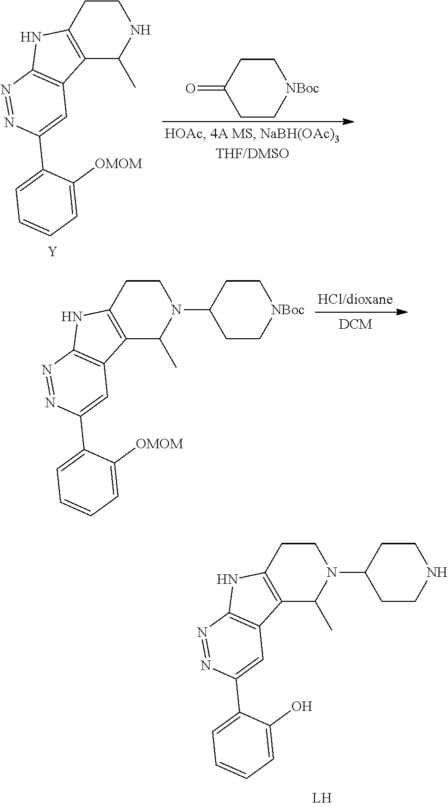

Step 1-Tert-butyl 4-(3-(2-(methoxymethoxy)phenyl)-5-methyl-7,8-dihydro-5H-pyrido[3′,4′:4,5]pyrrolo[2,3-c]pyridazin-6(9H)-yl)piperidine-1-carboxylate. To a solution of (3R)-12-[2-(methoxymethoxy)phenyl]-3-methyl-4,8,10,11-tetrazatricyclo[7.4.0.02,7]trideca-1(9),2(7),10,12-tetraene (4 g, 12 mmol, Intermediate Y) and tert-butyl 4-oxopiperidine-1-carboxylate (7.37 g, 37.0 mmol, CAS #79099-07-3) in DMSO (20 mL) was added HOAc (2.22 g, 37.0 mmol) and the mixture was stirred at 40° C. for 2 hours. Then NaBH(OAc)₃ (6.53 g, 30.8 mmol) was added and the mixture was stirred at 0-25° C. for 12 hrs. The reaction mixture was filtered and concentrated under reduced pressure to give a residue. The crude product was purified by reversed-phase HPLC (0.10% FA condition) to give the title compound (6.8 g, 94% yield, FA) as a yellow oil. ¹H NMR (400 MHz, DMSO-d₆) δ=12.03 (s, 1H), 8.15 (s, 1H), 7.95 (s, 1H), 7.72-7.67 (m, 1H), 7.42-7.36 (m, 1H), 7.24 (d, J=8.0 Hz, 1H), 7.13 (t, J=7.2 Hz, 1H), 5.75 (s, 1H), 5.23-5.16 (m, 2H), 4.22 (d, J=6.4 Hz, 1H), 3.92 (s, 3H), 3.11-3.05 (m, 1H), 3.00-2.95 (m, 1H), 2.88-2.62 (m, 7H), 1.88-1.68 (m, 3H), 1.38 (s, 9H), 1.35 (d, J=6.4 Hz, 3H); LC-MS (ESI+) m/z 508.2 (M+H)⁺.

Step 2-2-(5-methyl-6-(piperidin-4-yl)-6,7,8,9-tetrahydro-5H-pyrido[3′,4′:4,5]pyrrolo[2,3-c]pyridazin-3-yl)phenol. To a solution of tert-butyl 4-[12-[2-(methoxymethoxy)phenyl]-3-methyl-4,8,10,11-tetrazatricyclo[7.4.0.0^{2,7}]trideca-1(9),2(7),10,12-tetraen-4-yl]piperidine-1-carboxylate (1 g, 2 mmol) was added in DCM (10 mL) and HCl/dioxane (2 mL). The mixture was stirred at 25° C. for 1 hour. On completion, the reaction mixture was concentrated under reduced pressure to give the title compound (1 g, HCl) as a yellow solid. LC-MS (ESI+) m/z 364.1 (M+H)⁺.

### 2-(6-([1,4′-Bipiperidin]-4-yl)-5-methyl-6,7,8,9-tetrahydro-5H-pyrido[3′,4′:4,5]pyrrolo[2,3-c]pyridazin-3-yl)phenol (Intermediate LI)

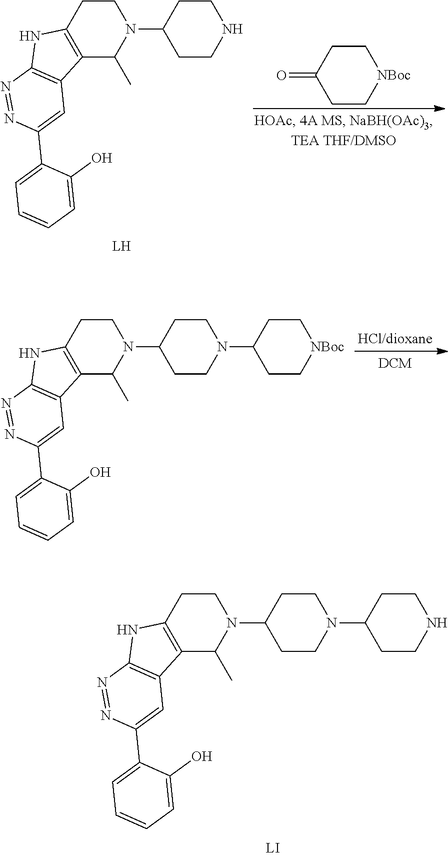 [SMILES: CC1c2c([nH]c3nnc(-c4ccccc4O)cc23)CCN1C1CCNCC1 ; CC(C)(C)OC(=O)N1CCC(=O)CC1 ; CC1c2c([nH]c3nnc(-c4ccccc4O)cc23)CCN1C1CCN(C2CCN(C(=O)OC(C)(C)C)CC2)CC1 ; CC1c2c([nH]c3nnc(-c4ccccc4O)cc23)CCN1C1CCN(C2CCNCC2)CC1]

Step 1-Tert-butyl 4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidine]-1′-carboxylate. To a solution of 2-[3-methyl-4-(4-piperidyl)-4,8,10,11-tetrazatricyclo[7.4.0.0^{2,7}]trideca-1(9),2(7),10,12-tetraen-12-yl]phenol (1 g, 3 mmol, Intermediate LH) in THF (20 mL) and DMSO (2 mL) was added TEA (557 mg, 5.50 mmol) and the mixture was stirred at 25° C. for 0.5 hour. Then tert-butyl 4-oxopiperidine-1-carboxylate (1.10 g, 5.50 mmol, CAS #79099-07-3) and HOAc (495.68 mg, 8.25 mmol) was added and stirred at 25° C. for 0.5 hour. Finally, NaBH(OAc)₃ (1.75 g, 8.25 mmol) was added and stirred at 0-25° C. for 12 hrs. The reaction mixture was filtered and concentrated under reduced pressure to give a residue. The crude product was purified by reversed-phase HPLC (0.1% FA condition) to give the title compound (600 mg, 39% yield, FA) as a yellow solid. LC-MS (ESI+) m/z 547.5 (M+H)⁺.

Step 2-2-(6-([1,4′-Bipiperidin]-4-yl)-5-methyl-6,7,8,9-tetrahydro-5H-pyrido[3′,4′:4,5]pyrrolo[2,3-c]pyridazin-3-yl)phenol. To a solution of tert-butyl 4-[4-[12-(2-hydroxyphenyl)-3-methyl-4,8,10,11-tetrazatricyclo[7.4.0.0^{2,7}]trideca-1(9),2(7),10,12-tetraen-4-yl]-1-piperidyl]piperidine-1-carboxylate (600 mg, 1.10 mmol) was added in HCl/dioxane (0.8 mL) and DCM (4 mL). The mixture was stirred at 25° C. for 1 hr. On completion, the reaction mixture was concentrated under reduced pressure to give the title compound (500 mg, HCl) as a yellow solid. LC-MS (ESI+) m/z 447.2 (M+H)⁺.

### (R)-Tert-butyl 6-(4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidin]-1′-yl)-2-azaspiro[3.3]heptane-2-carboxylate (Intermediate LJ) and (S)-tert-butyl 6-(4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidin]-1′-yl)-2-azaspiro[3.3]heptane-2-carboxylate (Intermediate LK)

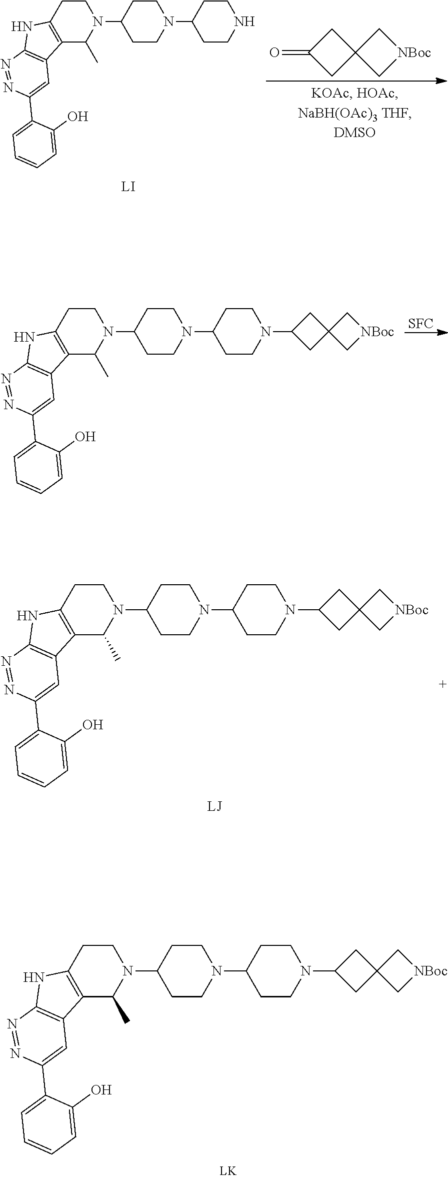

Step 1-Tert-butyl 6-(4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidin]-1′-yl)-2-azaspiro[3.3]heptane-2-carboxylate. To a solution of 2-[3-methyl-4-[1-(4-piperidyl)-4-piperidyl]-4,8,10,11-tetrazatricyclo[7.4.0.0^{2,7}]trideca-1(9),2(7),10,12-tetraen-12-yl]phenol (400 mg, 896 umol, Intermediate LI) in THF (5 mL) and DMSO (1 mL) was added KOAc (264 mg, 2.69 mmol) and the reaction was stirred at 25° C. for 0.5 hr. Then tert-butyl 6-oxo-2-azaspiro[3.3]heptane-2-carboxylate (227 mg, 1.07 mmol, CAS #1147557-97-8) and HOAc (215 mg, 3.58 mmol) was added and the mixture was stirred at 25° C. for 0.5 hr. Finally, NaBH(OAc)₃ (570 mg, 2.69 mmol) was added and the mixture was stirred at 0-25° C. for 12 hrs. On completion, the reaction mixture was filtered and concentrated under reduced pressure to give a residue. The crude product was purified by reversed-phase HPLC (0.1% FA condition) to give the title compound (440 mg, 76% yield, FA) as a yellow solid. LC-MS (ESI⁺) m/z 642.4 (M+H)⁺.

Step 2-(R)-tert-butyl 6-(4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidin]-1′-yl)-2-azaspiro[3.3]heptane-2-carboxylate and (S)-tert-butyl 6-(4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidin]-1′-yl)-2-azaspiro[3.3]heptane-2-carboxylate. Tert-butyl 6-[4-[4-[12-(2-hydroxyphenyl)-3-methyl-4,8,10,11-tetrazatricyclo[7.4.0.0^{2,7}]trideca-1(9),2(7),10,12-tetraen-4-yl]-1-piperidyl]-1-piperidyl]-2-azaspiro[3.3]heptane-2-carboxylate (440 mg, 686 umol) was separated by SFC (column: DAICEL CHIRALPAK AS (250 mm×30 mm, 10 um); mobile phase: [0.1% NH₃H₂O ETOH]; B %: 35%-35%, 7.0; 105 minutes) to give tert-butyl 6-[4-[4-[(3R)-12-(2-hydroxyphenyl)-3-methyl-4,8,10,11-tetrazatricyclo[7.4.0.0^{2,7}]trideca-1(9),2(7),10,12-tetraen-4-yl]-1-piperidyl]-1-piperidyl]-2-azaspiro[3.3]heptane-2-carboxylate (170 mg, 39% yield) as a yellow solid (LC-MS (ESI⁺) m/z 642.5 (M+H)⁺) and tert-butyl 6-[4-[4-[(3S)-12-(2-hydroxyphenyl)-3-methyl-4,8,10,11-tetrazatricyclo[7.4.0.0^{2,7}]trideca-1(9),2(7),10,12-tetraen-4-yl]-1-piperidyl]-1-piperidyl]-2-azaspiro[3.3]heptane-2-carboxylate (180 mg, 41% yield) as a yellow solid. LC-MS (ESI⁺) m/z 642.6 (M+H)⁺.

### (R)-2-(6-(1′-(2-azaspiro[3.3]heptan-6-yl)-[1,4′-bipiperidin]-4-yl)-5-methyl-6,7,8,9-tetrahydro-5H-pyrido[3′,4′:4,5]pyrrolo[2,3-c]pyridazin-3-yl)phenol (Intermediate LL)

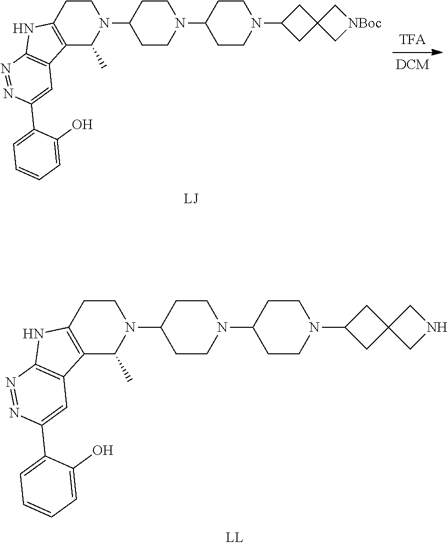 [SMILES: C[C@@H]1c2c([nH]c3nnc(-c4ccccc4O)cc23)CCN1C1CCN(C2CCN(C3CC4(C3)CN(C(=O)OC(C)(C)C)C4)CC2)CC1 ; C[C@@H]1c2c([nH]c3nnc(-c4ccccc4O)cc23)CCN1C1CCN(C2CCN(C3CC4(CNC4)C3)CC2)CC1]

To a solution of tert-butyl 6-[4-[4-[(3R)-12-(2-hydroxyphenyl)-3-methyl-4,8,10,11-tetrazatricyclo[7.4.0.0^{2,7}]trideca-1(9),2(7),10,12-tetraen-4-yl]-1-piperidyl]-1-piperidyl]-2-azaspiro[3.3]heptane-2-carboxylate (170 mg, 265 umol, Intermediate LJ) was added in TFA (0.2 mL) and DCM (2 mL). The mixture was stirred at 25° C. for 1 hour. On completion, the reaction mixture was filtered and concentrated under reduced pressure to give a residue. The crude product was purified by reversed-phase HPLC (0.1% FA condition) to give the title compound (150 mg, 96% yield, FA) as a yellow solid. LC-MS (ESI⁺) m/z 542.5 (M+H)⁺.

### (S)-2-(6-(1′-(2-azaspiro[3.3]heptan-6-yl)-[1,4′-bipiperidin]-4-yl)-5-methyl-6,7,8,9-tetrahydro-5H-pyrido[3′,4′:4,5]pyrrolo[2,3-c]pyridazin-3-yl)phenol (Intermediate LM)

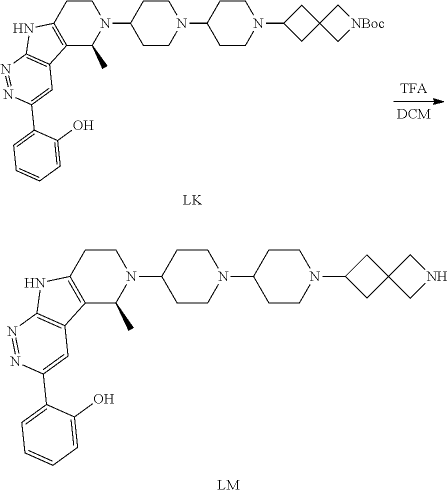 [SMILES: C[C@H]1c2c([nH]c3nnc(-c4ccccc4O)cc23)CCN1C1CCN(C2CCN(C3CC4(C3)CN(C(=O)OC(C)(C)C)C4)CC2)CC1 ; C[C@H]1c2c([nH]c3nnc(-c4ccccc4O)cc23)CCN1C1CCN(C2CCN(C3CC4(CNC4)C3)CC2)CC1]

To a solution of tert-butyl 6-[4-[4-[(3S)-12-(2-hydroxyphenyl)-3-methyl-4,8,10,11-tetrazatricyclo[7.4.0.0^{2,7}]trideca-1(9),2(7),10,12-tetraen-4-yl]-1-piperidyl]-1-piperidyl]-2-azaspiro[3.3]heptane-2-carboxylate (180 mg, 280 umol, Intermediate LK) was added in TFA (0.2 mL) and DCM (2 mL). The mixture was stirred at 25° C. for 1 hr. The reaction mixture was filtered and concentrated under reduced pressure to give a residue. The crude product was purified by reversed-phase HPLC (0.1% FA condition) to give the title compound (160 mg, 97% yield, FA) as a yellow solid. LC-MS (ESI+) m/z 542.5 (M+H)⁺.

### 2-[(3R)-3-methyl-4-[1-(4-piperidyl)-4-piperidyl]-4,8,10,11-tetrazatricyclo[7.4.0.0^{2,7}]trideca-1(9),2(7),10,12-tetraen-12-yl]phenol (Intermediate LN)

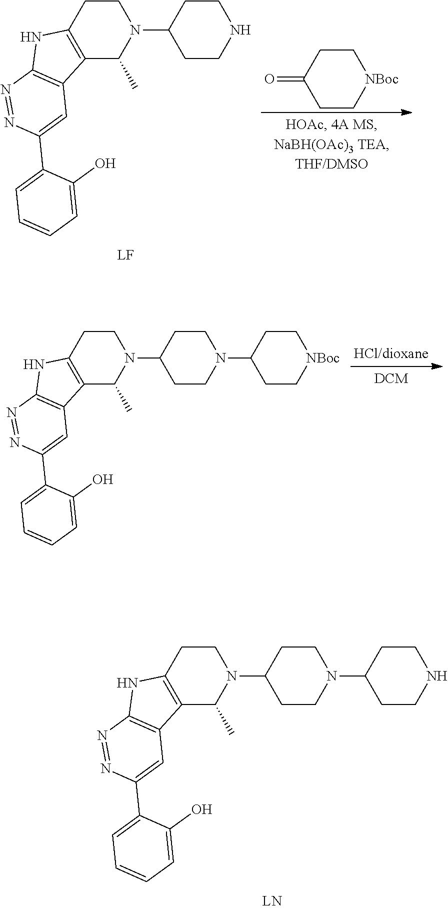 [SMILES: C[C@@H]1c2c([nH]c3nnc(-c4ccccc4O)cc23)CCN1C1CCNCC1 ; CC(C)(C)OC(=O)N1CCC(=O)CC1 ; C[C@@H]1c2c([nH]c3nnc(-c4ccccc4O)cc23)CCN1C1CCN(C2CCN(C(=O)OC(C)(C)C)CC2)CC1 ; C[C@@H]1c2c([nH]c3nnc(-c4ccccc4O)cc23)CCN1C1CCN(C2CCNCC2)CC1]

Step 1-(R)-tert-butyl 4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidine]-1′-carboxylate. To a solution of 2-[3-methyl-4-(4-piperidyl)-4,8,10,11-tetrazatricyclo[7.4.0.0^{2,7}]trideca-1(9),2(7),10,12-tetraen-12-yl]phenol (2 g, 5.00 mmol, Intermediate LF) in THF (20 mL) and DMSO (10 mL) was added TEA (1.01 mg, 10.0 mmol) and the mixture was stirred at 25° C. for 0.5 hr. Then tert-butyl 4-oxopiperidine-1-carboxylate (1.99 g, 10.0 mmol, CAS #7 9099-07-3) and HOAc (901 mg, 15.0 mmol) was added and the mixture was stirred at 25° C. for 0.5 hr. Finally, NaBH(OAc)₃ (2.65 g, 12.5 mmol) was added and the mixture was stirred at 0-25° C. for 12 hrs. The reaction mixture was filtered and concentrated under reduced pressure to give a residue. The crude product was purified by reversed-phase HPLC (0.1% FA condition) to give the title compound (1.8 g, 60% yield, FA) as a yellow solid. LC-MS (ESI+) m/z 547.5 (M+H)⁺.

Step 2-(R)-2-(6-([1,4′-bipiperidin]-4-yl)-5-methyl-6,7,8,9-tetrahydro-5H-pyrido[3′,4′:4,5]pyrrolo[2,3-c]pyridazin-3-yl)phenol. To a solution of tert-butyl 4-[4-[(3R)-12-(2-hydroxyphenyl)-3-methyl-4,8,10,11-tetrazatricyclo[7.4.0.02,7]trideca-1(9),2(7),10,12-tetraen-4-yl]-1-piperidyl]piperidine-1-carboxylate (1.8 g, 3.3 mmol) was added in DCM (20 mL) and HCl/dioxane (4M, 5 mL). The mixture was then stirred at 25° C. for 1 hr. On completion, the reaction mixture was concentrated under reduced pressure to give the title compound (1.6 g, HCl salt) as a yellow solid. LC-MS (ESI+) m/z 447.4 (M+H)⁺.

### 1-(((S)-1-((2S,4R)-2-((4-ethynylbenzyl)carbamoyl)-4-hydroxypyrrolidin-1-yl)-3,3-dimethyl-1-oxobutan-2-yl)carbamoyl)piperidine-4-carboxylic acid (Intermediate LO)

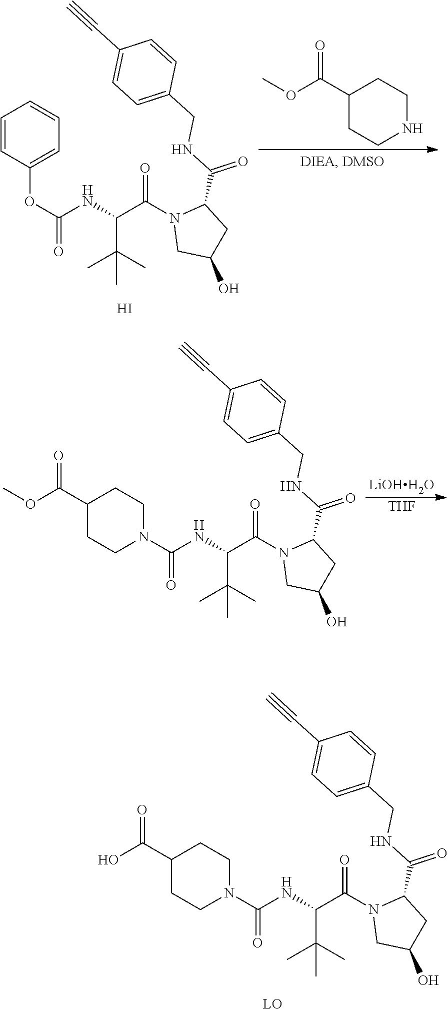

Step 1-Methyl 1-(((S)-1-((2S,4R)-2-((4-ethynylbenzyl)carbamoyl)-4-hydroxypyrrolidin-1-yl)-3,3-dimethyl-1-oxobutan-2-yl)carbamoyl)piperidine-4-carboxylate. To a solution of phenyl N-[(1S)-1-[(2S,4R)-2-[(4-ethynylphenyl)methylcarbamoyl]-4-hydroxy-pyrrolidine-1-carbonyl]-2,2-dimethyl-propyl]carbamate (500 mg, 1.05 mmol, Intermediate HI) and methyl piperidine-4-carboxylate;hydrochloride (263 mg, 1.22 mmol, CAS #2971-79-1) in DMSO (5 mL) was added DIEA (405 mg, 3.14 mmol). The mixture was stirred at 70° C. for 1 hour. The reaction mixture was directly purified. The crude product was purified by reversed-phase HPLC (0.1% FA condition) to give the title compound (400 mg, 67% yield) as a yellow solid. LC-MS (ESI+) m/z 527.3 (M+H)⁺.

Step 2-1-(((S)-1-((2S,4R)-2-((4-ethynylbenzyl)carbamoyl)-4-hydroxypyrrolidin-1-yl)-3,3-dimethyl-1-oxobutan-2-yl)carbamoyl)piperidine-4-carboxylic acid. To a solution of methyl 1-[[(1S)-1-[(2S, 4R)-2-[(4-ethynylphenyl) methylcarbamoyl]-4-hydroxy-pyrrolidine-1-carbonyl]-2,2-dimethyl-propyl]carbamoyl] piperidine-4-carboxylate (350 mg, 611 umol) in THF (4 mL) was added LiOH—H₂O (2 M, 2.21 mL). The mixture was stirred at 25° C. for 1 hour. On completion, the reaction mixture was filtered and concentrated under reduced pressure to give a residue. The residue was purified by reversed-phase HPLC (0.1% FA condition) to give the title compound (165 mg, 46% yield) as a white solid. LC-MS (ESI+) m/z 513.3 (M+H)⁺.

### (R)-2-(5-methyl-6-(1-(piperidin-4-ylmethyl)piperidin-4-yl)-6,7,8,9-tetrahydro-5H-pyrido[3′,4′:4,5]pyrrolo[2,3-c]pyridazin-3-yl)phenol (Intermediate LP)

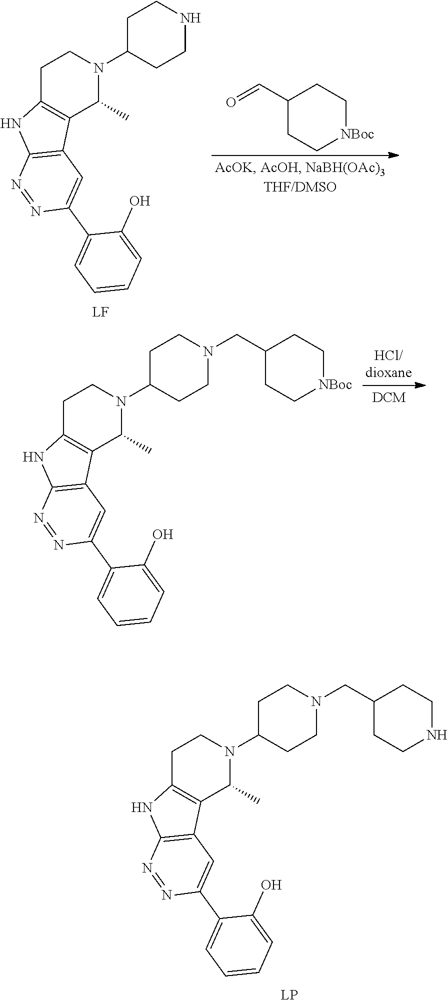 [SMILES: C[C@@H]1c2c([nH]c3nnc(-c4ccccc4O)cc23)CCN1C1CCNCC1 ; CC(C)(C)OC(=O)N1CCC(C=O)CC1 ; C[C@@H]1c2c([nH]c3nnc(-c4ccccc4O)cc23)CCN1C1CCN(CC2CCN(C(=O)OC(C)(C)C)CC2)CC1 ; C[C@@H]1c2c([nH]c3nnc(-c4ccccc4O)cc23)CCN1C1CCN(CC2CCNCC2)CC1]

Step 1-(R)-tert-butyl 4-((4-(3-(2-hydroxyphenyl)-5-methyl-7,8-dihydro-5H-pyrido[3′,4′:4,5]pyrrolo[2,3-c]pyridazin-6(9H)-yl)piperidin-1-yl)methyl)piperidine-1-carboxylate. To a solution of (R)-2-(5-methyl-6-(piperidin-4-yl)-6,7,8,9-tetrahydro-5H-pyrido[3′,4′:4,5]pyrrolo[2,3-c]pyridazin-3-yl)phenol (500 mg, 1.38 mmol, Intermediate LF) in THF (5 mL) and DMSO (1 mL) was added AcOK (405 mg, 4.13 mmol) at 25° C. and the mixture was stirred for 1 hr. Then AcOH (330 mg, 5.50 mmol) and tert-butyl 4-formylpiperidine-1-carboxylate (352 mg, 1.65 mmol) was added at 25° C. and the mixture was stirred for 2 hrs. Last, NaBH(OAc)₃ (875 mg, 4.13 mmol) was added at 0° C. and the mixture was stirred at 25° C. for 12 h. On completion, the reaction mixture was concentrated in vacuo to give a residue. The crude product was purified by reversed-phase HPLC (0.1% FA condition) to give the title compound (400 mg, 44% yield, FA) as a yellow oil. LC-MS (ESI⁺) m/z 561.4 (M+H)⁺.

Step 2-(R)-2-(5-methyl-6-(1-(piperidin-4-ylmethyl)piperidin-4-yl)-6,7,8,9-tetrahydro-5H-pyrido[3′,4′:4,5]pyrrolo[2,3-c]pyridazin-3-yl)phenol. To a mixture of (R)-tert-butyl 4-((4-(3-(2-hydroxyphenyl)-5-methyl-7,8-dihydro-5H-pyrido [3′,4′:4,5]pyrrolo[2,3-c]pyridazin-6(9H)-yl)piperidin-1-yl)methyl)piperidine-1-carboxylate (100 mg, 178 umol) in DCM (1 mL) was added HCl/dioxane (4 M, 0.2 mL). The mixture was then stirred at 25° C. for 30 minutes. On completion, the reaction mixture was concentrated in vacuo to give a residue. The crude product was purified by reversed-phase HPLC (0.1% FA condition) to give the title compound (70 mg, 78% yield, FA) as a yellow solid. LC-MS (ESI⁺) m z 461.3 (M+H)⁺.

### (R)-2-(5-methyl-6-(2-azaspiro[3.3]heptan-6-yl)-6,7,8,9-tetrahydro-5H-pyrido[3′,4′:4,5]pyrrolo[2,3-c]pyridazin-3-yl)phenol (Intermediate LQ)

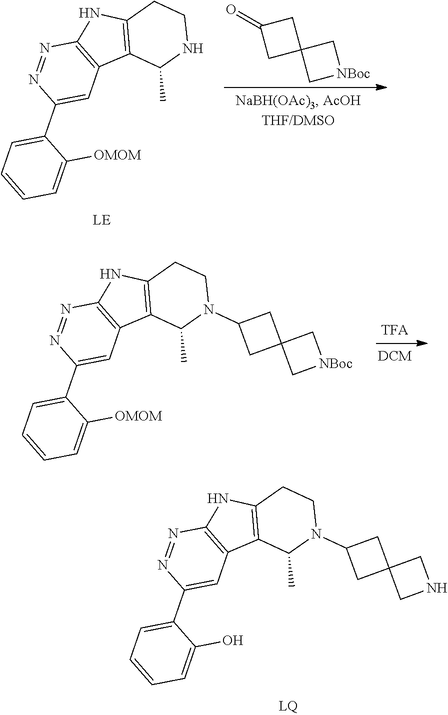

Step 1-(R)-tert-butyl 6-(3-(2-(methoxymethoxy)phenyl)-5-methyl-7,8-dihydro-5H-pyrido[3′,4′:4,5]pyrrolo[2,3-c]pyridazin-6(9H)-yl)-2-azaspiro[3.3]heptane-2-carboxylate. To a solution of (3R)-12-[2-(methoxymethoxy)phenyl]-3-methyl-4,8,10,11-tetrazatricyclo[7.4.0.02,7]trideca-1(9),2(7),10,12-tetraene (800 mg, 2.47 mmol, Intermediate LE) in THF (20 mL), DMSO (4 mL) was added AcOH (444 mg, 7.40 mmol, 423 uL), and tert-butyl 6-oxo-2-azaspiro[3.3]heptane-2-carboxylate (625 mg, 2.96 mmol, CAS #1147557-97-8) at 25° C. for 1.5 hr. Then NaBH(OAc)₃ (1.57 g, 7.40 mmol) was added at 0° C. The mixture was then stirred at 25° C. for 2.5 hr. On completion, the reaction was added water (2 ml), and then concentrated under reduced pressure to give the residue. The residue was purified by prep-HPLC (FA) to give the title compound (2.03 g, 72% yield) as a yellow solid. LC-MS (ESI⁺) m/z 520.5 (M+H)⁺.

Step 2-(R)-2-(5-methyl-6-(2-azaspiro[3.3]heptan-6-yl)-6,7,8,9-tetrahydro-5H-pyrido[3′,4′:4,5]pyrrolo[2,3-c]pyridazin-3-yl)phenol. To a solution of tert-butyl 6-[(3R)-12-[2-(methoxymethoxy)phenyl]-3-methyl-4,8,10,11-tetrazatricyclo[7.4.0.02,7]trideca-1(9),2(7),10,12-tetraen-4-yl]-2-azaspiro[3.3]heptane-2-carboxylate (600 mg, 1 mmol, FA) in DCM (6 mL) was added TFA (3.40 g, 29.7 mmol, 2.20 mL). The mixture was then stirred at 25° C. for 2 hr. On completion, the reaction was concentrated under reduced pressure to give the residue. The residue was purified by prep-HPLC (FA) to give the title compound (304 mg, 68% yield) as a white solid. LC-MS (ESI⁺) m/z 376.1 (M+H)⁺.

### (R)-2-(6-(2-(1-(2-azaspiro[3.3]heptan-6-yl)piperidin-4-yl)-2-azaspiro[3.3]heptan-6-yl)-5-methyl-6,7,8,9-tetrahydro-5H-pyrido[3′,4′:4,5]pyrrolo[2,3-c]pyridazin-3-yl)phenol (Intermediate LR)

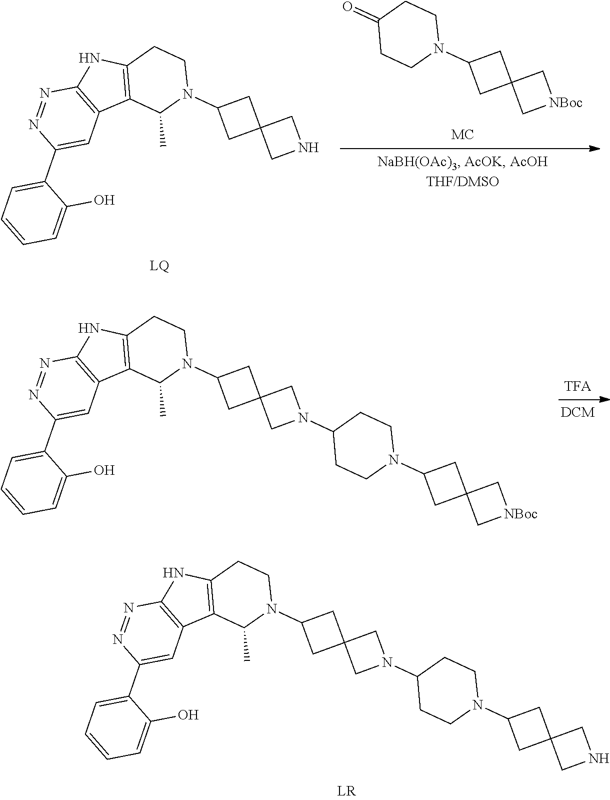 [SMILES: C[C@@H]1c2c([nH]c3nnc(-c4ccccc4O)cc23)CCN1C1CC2(CNC2)C1 ; CC(C)(C)OC(=O)N1CC2(CC(N3CCC(=O)CC3)C2)C1 ; C[C@@H]1c2c([nH]c3nnc(-c4ccccc4O)cc23)CCN1C1CC2(C1)CN(C1CCN(C3CC4(C3)CN(C(=O)OC(C)(C)C)C4)CC1)C2 ; C[C@@H]1c2c([nH]c3nnc(-c4ccccc4O)cc23)CCN1C1CC2(C1)CN(C1CCN(C3CC4(CNC4)C3)CC1)C2]

Step 1-(R)-tert-butyl 6-(4-(6-(3-(2-hydroxyphenyl)-5-methyl-7,8-dihydro-5H-pyrido[3′,4′:4,5]pyrrolo[2,3-c]pyridazin-6(9H)-yl)-2-azaspiro[3.3]heptan-2-yl)piperidin-1-yl)-2-azaspiro[3.3]heptane-2-carboxylate. To a solution of 2-[(3R)-4-(2-azaspiro[3.3]heptan-6-yl)-3-methyl-4,8,10,11-tetrazatricyclo[7.4.0.02,7]trideca-1(9),2(7),10,12-tetraen-12-yl]phenol (1.1 g, 2.93 mmol, Intermediate LQ) in THF (20 mL), DMSO (4 mL) was added AcOK (862 mg, 8.79 mmol) at 25° C. for 0.5 hr. Then, AcOH (527 mg, 8.79 mmol, 502 uL), and tert-butyl 6-(4-oxo-1-piperidyl)-2-azaspiro[3.3]heptane-2-carboxylate (948 mg, 3.22 mmol, Intermediate MC) was added and the mixture was stirred at 25° C. for 1.5 hr. Finally, to the solution was added NaBH(OAc)₃ (1.86 g, 8.79 mmol) at 0° C. The mixture was stirred at 25° C. for 12 hr. On completion, the reaction was added water (2 ml), and then concentrated under reduced pressure to give the residue. The residue was purified by prep-HPLC (FA) to give the title compound (1.34 g, 64% yield) as a white solid. LC-MS (ESI⁺) m/z 654.4 (M+H)⁺.

Step 2-(R)-2-(6-(2-(1-(2-azaspiro[3.3]heptan-6-yl)piperidin-4-yl)-2-azaspiro[3.3]heptan-6-yl)-5-methyl-6,7,8,9-tetrahydro-5H-pyrido[3′,4′:4,5]pyrrolo[2,3-c]pyridazin-3-yl)phenol. To a solution of tert-butyl 6-[4-[6-[(3R)-12-(2-hydroxyphenyl)-3-methyl-4,8,10,11-tetrazatricyclo[7.4.0.02,7]trideca-1(9),2(7),10,12-tetraen-4-yl]-2-azaspiro[3.3]heptan-2-yl]-1-piperidyl]-2-azaspiro[3.3]heptane-2-carboxylate (1.3 g, 2.0 mmol) in DCM (40 mL) was added TFA (12.3 g, 108 mmol, 8 mL). The mixture was stirred at 25° C. for 4 hr. On completion, the reaction was concentrated under reduced pressure to give the residue. The residue was purified by prep-HPLC (FA) to give the title compound (834 mg, 70% yield) as a white solid. LC-MS (ESI⁺) m/z 554.2 (M+H)⁺.

### 2-((R)-5-methyl-6-((1r,4R)-4-(piperazin-1-yl)cyclohexyl)-6,7,8,9-tetrahydro-5H-pyrido[3′,4′:4,5]pyrrolo[2,3-c]pyridazin-3-yl)phenol (Intermediate LS)

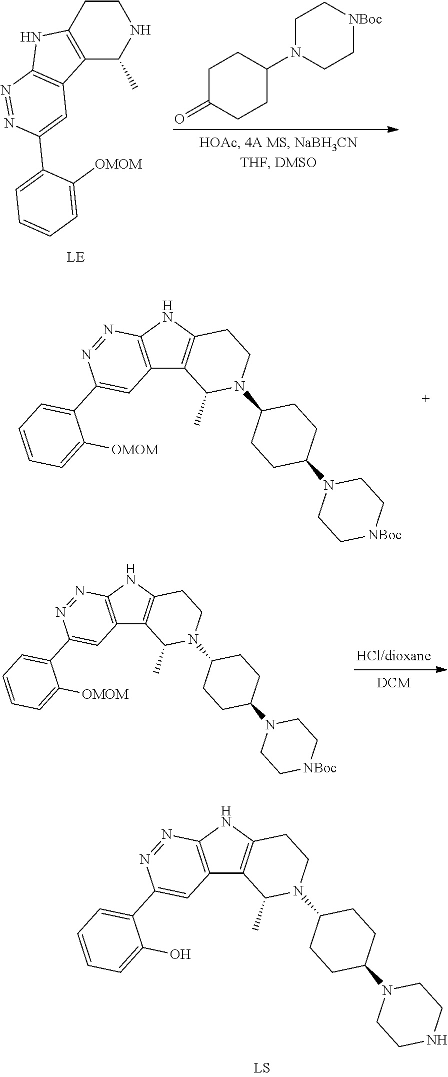

Step 1-Tert-butyl 4-((1R,4r)-4-((R)-3-(2-(methoxymethoxy)phenyl)-5-methyl-7,8-dihydro-5H-pyrido[3′,4′:4,5]pyrrolo[2,3-c]pyridazin-6(9H)-yl)cyclohexyl)piperazine-1-carboxylate and tert-butyl 4-((1S,4s)-4-((R)-3-(2-(methoxymethoxy)phenyl)-5-methyl-5,7,8,9-tetrahydro-6H-pyrido[3′,4′:4,5]pyrrolo[2,3-c]pyridazin-6-yl)cyclohexyl)piperazine-1-carboxylate. To a solution of (3R)-12-[2-(methoxymethoxy)phenyl]-3-methyl-4,8,10,11-tetrazatricyclo[7.4.0.0^{2,7}]trideca-1(9),2(7),10,12-tetraene (884.52 mg, 2.73 mmol, Intermediate LE) in THF (16 mL) and DMSO (4 mL) was added HOAc (164 mg, 2.73 mmol, 156 uL) and 4 Å molecular sieves (900 mg, 2.73 mmol) tert-butyl 4-(4-oxocyclohexyl)piperazine-1-carboxylate (770 mg, 2.73 mmol, CAS #1262409-94-8). After 1 hour NaBH₃CN (514.08 mg, 8.18 mmol) was added and the mixture was stirred at 0-25° C. for 12 hrs. On completion, the reaction mixture was partitioned between EA (100 mL) and H₂O (80 mL). The organic phase was separated, washed with NaCl_{(aq)} (20 mL×3), dried over Na₂SO₄, filtered and concentrated under reduced pressure to give a residue. The crude product was purified by reversed-phase HPLC(0.1% FA condition) to give Tert-butyl 4-((1R,4r)-4-((R)-3-(2-(methoxymethoxy)phenyl)-5-methyl-7,8-dihydro-5H-pyrido[3′,4′:4,5]pyrrolo[2,3-c]pyridazin-6(9H)-yl)cyclohexyl)piperazine-1-carboxylate (180 mg, 10% yield) and tert-butyl 4-((1S,4s)-4-((R)-3-(2-(methoxymethoxy)phenyl)-5-methyl-5,7,8,9-tetrahydro-6H-pyrido[3′,4′:4,5]pyrrolo[2,3-c]pyridazin-6-yl)cyclohexyl)piperazine-1-carboxylate (285 mg, 18% yield, as a yellow solids. LC-MS (ESI⁺) m/z 591.5(M+H)⁺.

Step 2-2-((R)-5-methyl-6-((1r,4R)-4-(piperazin-1-yl)cyclohexyl)-6,7,8,9-tetrahydro-5H-pyrido[3′,4′:4,5]pyrrolo[2,3-c]pyridazin-3-yl)phenol. To a solution of tert-butyl 4-[4-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]cyclohexyl]piperazine-1-carboxylate (130 mg, 220 umol) in DCM (2 mL) was added HCl/dioxane (1 M, 220.06 uL). The mixture was then stirred at 25° C. for 1 hour. On completion, the reaction mixture was concentrated under reduced pressure to give the title compound (100 mg) as a white solid. LC-MS (ESI+) m/z 447.3 (M+H)⁺.

### 2-((R)-6-((1r,4R)-4-(4-(2-azaspiro[3.3]heptan-6-yl)piperazin-1-yl)cyclohexyl)-5-methyl-6,7,8,9-tetrahydro-5H-pyrido[3′,4′:4,5]pyrrolo[2,3-c]pyridazin-3-yl)phenol (Intermediate LT)

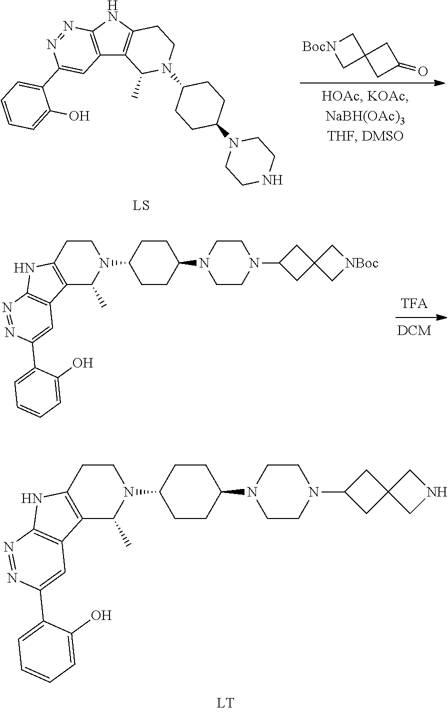 [SMILES: C[C@@H]1c2c([nH]c3nnc(-c4ccccc4O)cc23)CCN1[C@H]1CC[C@H](N2CCNCC2)CC1 ; CC(C)(C)OC(=O)N1CC2(CC(=O)C2)C1 ; C[C@@H]1c2c([nH]c3nnc(-c4ccccc4O)cc23)CCN1[C@H]1CC[C@H](N2CCN(C3CC4(C3)CN(C(=O)OC(C)(C)C)C4)CC2)CC1 ; C[C@@H]1c2c([nH]c3nnc(-c4ccccc4O)cc23)CCN1[C@H]1CC[C@H](N2CCN(C3CC4(CNC4)C3)CC2)CC1]

Step 1-Tert-butyl 6-(4-((1R,4r)-4-((R)-3-(2-hydroxyphenyl)-5-methyl-7,8-dihydro-5H-pyrido[3′,4′:4,5]pyrrolo[2,3-c]pyridazin-6(9H)-yl)cyclohexyl)piperazin-1-yl)-2-azaspiro[3.3]heptane-2-carboxylate. To a solution of tert-butyl 6-oxo-2-azaspiro[3.3]heptane-2-carboxylate (47.30 mg, 224 umol,) in THF (2.5 mL) and DMSO (0.6 mL) was added KOAc (65.9 mg, 672 umol). After 1 hour of stirring at rt, 2-[(3R)-3-methyl-4-(4-piperazin-1-ylcyclohexyl)-4,8,10,11-tetrazatricyclo[7.4.0.02,7]trideca-1(9),2(7),10,12-tetraen-12-yl]phenol (100 mg, 200 umol, Intermediate LS) HOAc (53.8 mg, 896 umol, 51.2 uL), and 4 Å molecular sieves (100 mg, 200 umol). After 1 hr of stirring at rt, NaBH(OAc)₃ (142.37 mg, 671.76 umol) was added at 0° C. The mixture was then stirred at 0-25° C. for 12 hrs. On completion, the reaction mixture was partitioned between EA (10 mL) and H₂O (10 mL). The organic phase was separated, washed with NaCl 9 mL (3 mL×3), dried over Na₂SO₄, filtered and concentrated under reduced pressure to give a residue. The residue was purified by column chromatography (SiO₂, Petroleum ether/Ethyl acetate=50/1 to 15/1) to give the title compound (170 mg, 57% yield) as a yellow solid. LC-MS (ESI⁺) m/z 642.8 (M+H)⁺.

Step 2-2-((R)-6-((1r,4R)-4-(4-(2-azaspiro[3.3]heptan-6-yl)piperazin-1-yl)cyclohexyl)-5-methyl-6,7,8,9-tetrahydro-5H-pyrido[3′,4′:4,5]pyrrolo[2,3-c]pyridazin-3-yl)phenol. To a solution of To a solution of tert-butyl 6-[4-[4-[(3R)-12-(2-hydroxyphenyl)-3-methyl-4,8,10,11-tetrazatricyclo[7.4.0.02,7]trideca-1(9),2(7),10,12-tetraen-4-yl]cyclohexyl]piperazin-1-yl]-2-azaspiro[3.3]heptane-2-carboxylate (170 mg, 264.86 umol) in DCM (5.5 mL) was added TFA (847.00 mg, 7.43 mmol, 550.00 uL). The mixture was stirred at 25° C. for 1 hr. On completion, the reaction mixture was concentrated under reduced pressure to remove solvent. The crude product was purified by reversed-phase HPLC (0.1% FA condition) to give the title compound (145 mg, 51% yield) as a yellow solid. LC-MS (ESI⁺) m/z 542.6 (M+H)⁺.

### 2-((R)-5-methyl-6-((1s,4S)-4-(piperazin-1-yl)cyclohexyl)-6,7,8,9-tetrahydro-5H-pyrido[3′,4′:4,5]pyrrolo[2,3-c]pyridazin-3-yl)phenol (Intermediate LU)

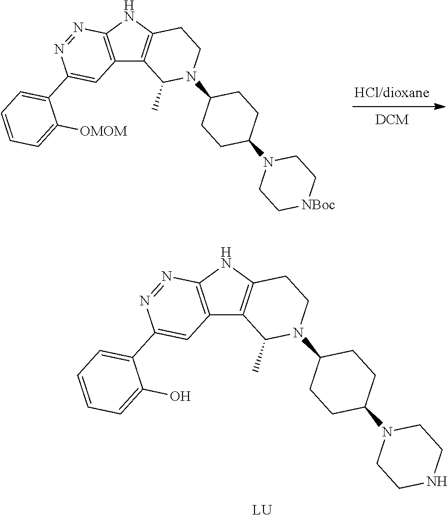

To a solution of tert-butyl 4-[4-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]cyclohexyl]piperazine-1-carboxylate (285 mg, 482 umol, synthesized via Step 1 of Intermediate LS) in DCM (3 mL) was added HCl/dioxane (1 M, 482 uL). The mixture was stirred at 25° C. for 1 hr. On completion, the reaction mixture was concentrated under reduced pressure to give the title compound (215 mg, HCl) as a white solid. LC-MS (ESI⁺) m/z 447.2 (M+H)⁺.

### 2-((R)-6-((1s,4S)-4-(4-(2-Azaspiro[3.3]heptan-6-yl)piperazin-1-yl)cyclohexyl)-5-methyl-6,7,8,9-tetrahydro-5H-pyrido[3′,4′:4,5]pyrrolo[2,3-c]pyridazin-3-yl)phenol (Intermediate LV)

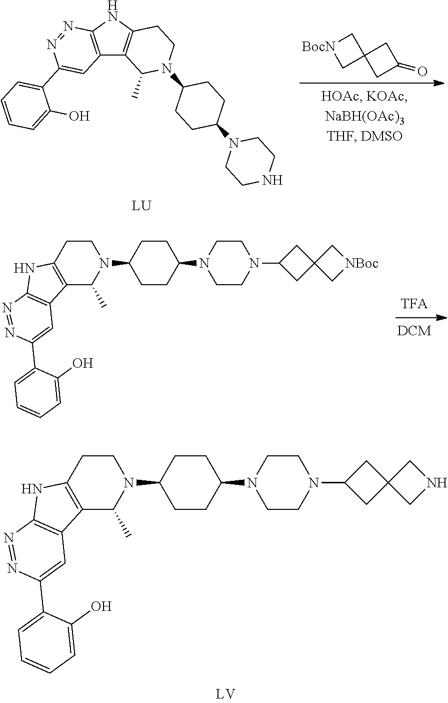 [SMILES: C[C@@H]1c2c([nH]c3nnc(-c4ccccc4O)cc23)CCN1[C@H]1CC[C@@H](N2CCNCC2)CC1 ; CC(C)(C)OC(=O)N1CC2(CC(=O)C2)C1 ; C[C@@H]1c2c([nH]c3nnc(-c4ccccc4O)cc23)CCN1[C@H]1CC[C@@H](N2CCN(C3CC4(C3)CN(C(=O)OC(C)(C)C)C4)CC2)CC1 ; C[C@@H]1c2c([nH]c3nnc(-c4ccccc4O)cc23)CCN1[C@H]1CC[C@@H](N2CCN(C3CC4(CNC4)C3)CC2)CC1]

Step 1-Tert-butyl 6-(4-((1S,4s)-4-((R)-3-(2-hydroxyphenyl)-5-methyl-7,8-dihydro-5H-pyrido[3′,4′:4,5]pyrrolo[2,3-c]pyridazin-6(9H)-yl)cyclohexyl)piperazin-1-yl)-2-azaspiro[3.3]heptane-2-carboxylate. To a solution of 2-[(3R)-3-methyl-4-(4-piperazin-1-ylcyclohexyl)-4,8,10,11-tetrazatricyclo[7.4.0.0^{2,7}]trideca-1(9),2(7),10,12-tetraen-12-yl]phenol (215.00 mg, 481 umol, Intermediate LU) in THF (6 mL) DMSO (1.5 mL) was added KOAc (142 mg, 1.44 mmol). After 1 hr of stirring at rt, tert-butyl 6-oxo-2-azaspiro[3.3]heptane-2-carboxylate (101.71 mg, 481 umol), HOAc (164 mg, 1.93 mmol, 110 uL), 4 Å molecular sieves (215 mg, 481 umol) was added and the mixture was stirred at rt for 1 hr. Next, NaBH(OAc)₃ (306 mg, 1.44 mmol) was added to the reaction mixture at 0° C. The mixture was stirred at 0-25° C. for 12 hrs. On completion, the reaction mixture was partitioned between EA (10 mL) and H₂O (10 mL). The organic phase was separated, washed with NaCl 9 mL (3 mL×3), dried over Na₂SO₄, filtered and concentrated under reduced pressure to give a residue. The residue was purified by column chromatography (SiO₂, Petroleum ether/Ethyl acetate=50/1 to 15/1) to give the title compound (230 mg, 72% yield) as a yellow solid. LC-MS (ESI⁺) m/z 642.6(M+H)⁺.

Step 2-2-((R)-6-((1s,4S)-4-(4-(2-azaspiro[3.3]heptan-6-yl)piperazin-1-yl)cyclohexyl)-5-methyl-6,7,8,9-tetrahydro-5H-pyrido[3′,4′:4,5]pyrrolo[2,3-c]pyridazin-3-yl)phenol. To a solution of tert-butyl 6-[4-[4-[(3R)-12-(2-hydroxyphenyl)-3-methyl-4,8,10,11-tetrazatricyclo[7.4.0.0^{2,7}]trideca-1(9),2(7),10,12-tetraen-4-yl]cyclohexyl]piperazin-1-yl]-2-azaspiro[3.3]heptane-2-carboxylate (204 mg, 318 umol) in DCM (7 mL) was added TFA (1.08 g, 9.45 mmol, 700.00 uL). The mixture was stirred at 25° C. for 1 hr. On completion, the reaction mixture was concentrated under reduced pressure to remove solvent. The crude product was purified by reversed-phase HPLC(0.1% FA condition) to give the title compound (270 mg, 72% yield, FA) as a yellow solid. LC-MS (ESI+) m/z542.6 (M+H)⁺.

### (R)-2-(6-(1′-(2-azaspiro[3.3]heptan-6-ylmethyl)-[1,4′-bipiperidin]-4-yl)-5-methyl-6,7,8,9-tetrahydro-5H-pyrido[3′,4′:4,5]pyrrolo[2,3-c]pyridazin-3-yl)phenol (Intermediate LW)

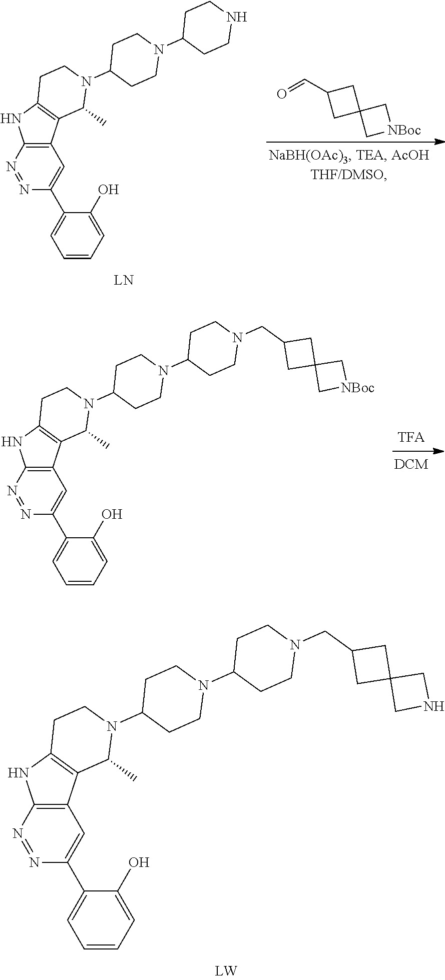 [SMILES: C[C@@H]1c2c([nH]c3nnc(-c4ccccc4O)cc23)CCN1C1CCN(C2CCNCC2)CC1 ; CC(C)(C)OC(=O)N1CC2(CC(C=O)C2)C1 ; C[C@@H]1c2c([nH]c3nnc(-c4ccccc4O)cc23)CCN1C1CCN(C2CCN(CC3CC4(C3)CN(C(=O)OC(C)(C)C)C4)CC2)CC1 ; C[C@@H]1c2c([nH]c3nnc(-c4ccccc4O)cc23)CCN1C1CCN(C2CCN(CC3CC4(CNC4)C3)CC2)CC1]

Step 1-(R)-tert-butyl 6-((4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidin]-1′-yl)methyl)-2-azaspiro[3.3]heptane-2-carboxylate. To a solution of 2-[(3R)-3-methyl-4-[1-(4-piperidyl)-4-piperidyl]-4,8,10,11-tetrazatricyclo[7.4.0.02,7]trideca-1(9),2(7),10,12-tetraen-12-yl]phenol (200 mg, 448 umol, Intermediate LN) in THF (4 mL) and DMSO (2 mL) was added TEA (136 mg, 1.34 mmol, 187 uL) and stirred at 25° C. for 0.5 hrs. Then AcOH (80.7 mg, 1.34 mmol, 76.8 uL) and tert-butyl 6-formyl-2-azaspiro [3.3] heptane-2-carboxylate (141 mg, 627 umol, CAS #1440960-67-7) was added and the mixture was stirred for another 1.5 hrs. Finally, NaBH(OAc)₃ (285 mg, 1.34 mmol) was added at 0° C. and the mixture was stirred at 25° C. for 3.5 hours. On completion, to the reaction was added water (1 ml), and then the mixture was concentrated under reduced pressure to give a residue. The residue was purified by prep-HPLC (FA) to give the title compound (140 mg, 45% yield) as a white solid. LC-MS (ESI⁺) m/z 656.6 (M+H)⁺.

Step 2-(R)-2-(6-(1′-(2-azaspiro[3.3]heptan-6-ylmethyl)-[1,4′-bipiperidin]-4-yl)-5-methyl-6,7,8,9-tetrahydro-5H-pyrido[3′,4′:4,5]pyrrolo[2,3-c]pyridazin-3-yl)phenol. To a solution of tert-butyl 6-[[4-[4-[(3R)-12-(2-hydroxyphenyl)-3-methyl-4, 8, 10, 11-tetrazatricyclo [7.4.0.02,7] trideca-1(9),2(7),10,12-tetraen-4-yl]-1-piperidyl]-1-piperidyl]methyl]-2-azaspiro[3.3]heptane-2-carboxylate (140 mg, 210 umol) in DCM (6 mL) was added TFA (2.16 g, 18.9 mmol, 1.40 mL). The mixture was stirred at 25° C. for 2 hours. On completion, the reaction was concentrated under reduced pressure to give the residue. The residue was purified by prep-HPLC (FA) to give the title compound (120 mg, 93% yield) as a yellow solid. LC-MS (ESI⁺) m/z 556.4 (M+H)⁺.

### (2S,4R)-1-((S)-3,3-dimethyl-2-(2-oxospiro[3.5]nonane-7-carboxamido)butanoyl)-N-(4-ethynyl-2-methoxybenzyl)-4-hydroxypyrrolidine-2-carboxamide (Intermediate LX)

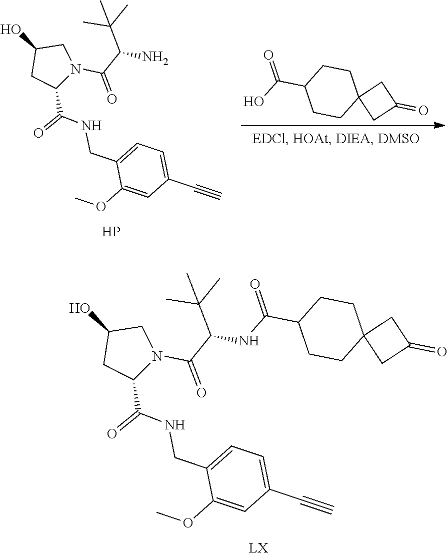

A mixture of (2S,4R)-1-[(2S)-2-amino-3,3-dimethyl-butanoyl]-N-[(4-ethynyl-2-methoxy-phenyl)methyl]-4-hydroxy-pyrrolidine-2-carboxamide (600 mg, 2 mmol, Intermediate HP), 2-oxospiro[3.5]nonane-7-carboxylic acid (282 mg, 1.55 mmol, CAS #2167815-01-0), EDCI (742 mg, 3.87 mmol), HOAt (527 mg, 3.87 mmol) and DIEA (1.00 g, 7.74 mmol) in DMSO (6 mL) was degassed and purged with N₂ three times. Then the mixture was stirred at 25° C. for 12 hrs under N₂ atmosphere. On completion, the reaction mixture was concentrated under reduced pressure to remove EA. The residue was diluted with water (10 mL) and extracted with EA (10 mL×3). The combined organic layers were washed with brine (10 mL×5), dried over Na₂SO₄, filtered and concentrated under reduced pressure to give a residue. The residue was purified by column chromatography (SiO₂, Petroleum ether/Ethyl acetate=1/0 to 1/2) to give the title compound (590 mg, 66% yield) as a white solid. LC-MS (ESI⁺) m/z 552.5 (M+H)⁺.

### (R)-4-(4-(3-(2-hydroxyphenyl)-5-methyl-7,8-dihydro-5H-pyrido[3′,4′:4,5]pyrrolo[2,3-c]pyridazin-6(9H)-yl)piperidin-1-yl)cyclohexanecarbaldehyde (Intermediate LY)

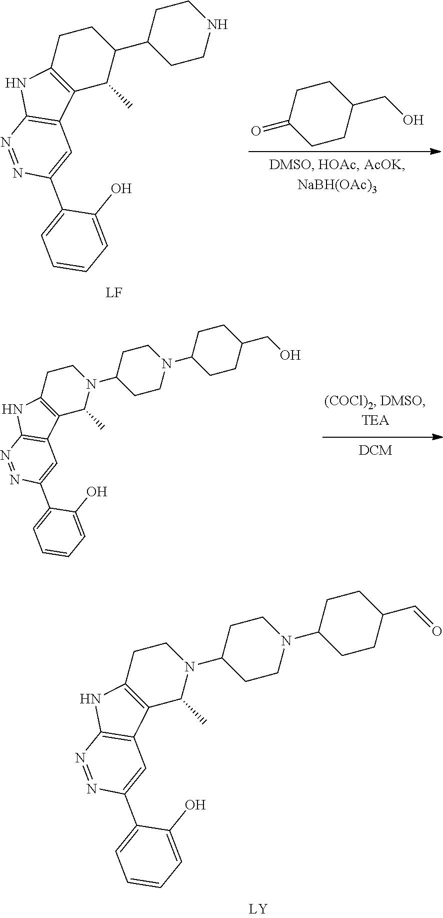 [SMILES: C[C@@H]1c2c([nH]c3nnc(-c4ccccc4O)cc23)CCC1C1CCNCC1 ; O=C1CCC(CO)CC1 ; C[C@@H]1c2c([nH]c3nnc(-c4ccccc4O)cc23)CCN1C1CCN(C2CCC(CO)CC2)CC1 ; C[C@@H]1c2c([nH]c3nnc(-c4ccccc4O)cc23)CCN1C1CCN(C2CCC(C=O)CC2)CC1]

Step 1-(R)-2-(6-(1-(4-(hydroxymethyl)cyclohexyl)piperidin-4-yl)-5-methyl-6,7,8,9-tetrahydro-5H-pyrido[3′,4′:4,5]pyrrolo[2,3-c]pyridazin-3-yl)phenol. To a solution of 2-[(3R)-3-methyl-4-(4-piperidyl)-4,8,10,11-tetrazatricyclo[7.4.0.02,7]trideca-1(9),2(7),10,12-tetraen-12-yl]phenol (700 mg, 2 mmol, Intermediate LF) in DMSO (12 mL) was added KOAc (515 mg, 5.25 mmol) at 25° C. for 30 min. Then the mixture HOAc (315 mg, 5.25 mmol, 300 uL) and 4-(hydroxymethyl)cyclohexanone (673 mg, 5.25 mmol) was added and the mixture was stirred at 40° C. for 2 hrs. Finally, NaBH(OAc)₃ (927 mg, 4.38 mmol) was added and the mixture was stirred at 25° C. for 12 hrs. On completion, the mixture was diluted with DMSO (15 mL). The residue was purified by prep-HPLC (0.1% NH₃.H₂O) to give the title compound (300 mg, 33% yield) as a yellow solid. LC-MS (ESI⁺) m/z 476.3 (M+H)⁺.

Step 2-(R)-4-(4-(3-(2-hydroxyphenyl)-5-methyl-7,8-dihydro-5H-pyrido[3′,4′:4,5]pyrrolo[2,3-c]pyridazin-6(9H)-yl)piperidin-1-yl)cyclohexanecarbaldehyde. To a solution of (COCl)₂ (42.7 mg, 336 umol, 29.4 uL) in DCM (0.5 mL) was added DMSO (39.4 mg, 504 umol, 39.4 uL) at −78° C. and the mixture was stirred for 2 hrs. Then 2-[(3R)-4-[1-[4-(hydroxymethyl)cyclohexyl]-4-piperidyl]-3-methyl-4,8,10,11-tetrazatricyclo[7.4.0.02,7]trideca-1(9),2(7),10,12-tetraen-12-yl]phenol (80 mg, 168 umol) in DCM (0.5 mL) was added at −78° C. and the mixture was stirred for 2 hrs. Finally, TEA (85.1 mg, 841 umol, 117 uL) in DCM (0.5 mL) was added and the mixture was stirred at 25° C. for 2 hrs. On completion, the reaction mixture was concentrated under reduced pressure to give the title compound (80 mg) as a yellow solid. LC-MS (ESI⁺) m/z 474.4 (M+H)⁺

(S)-2-(5-methyl-6-(4-(piperazin-1-ylmethyl)cyclohexyl)-6,7,8,9-tetrahydro-5H-pyrido[3′,4′:4,5]pyrrolo[2,3-c]pyridazin-3-yl)phenol (Intermediate LZ)

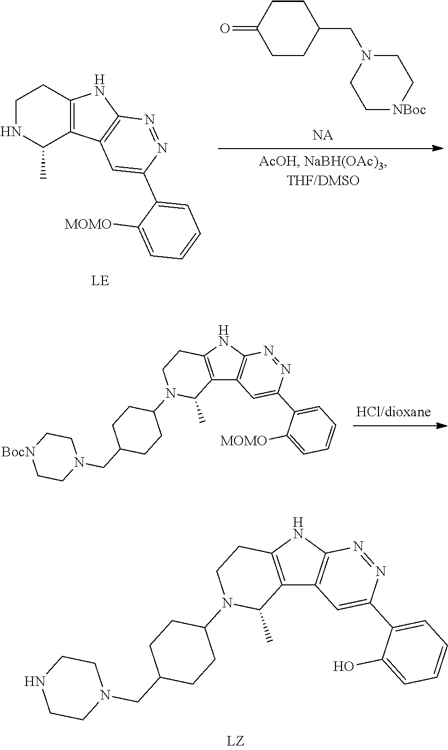

Step 1-(S)-tert-butyl 4-((4-(3-(2-(methoxymethoxy)phenyl)-5-methyl-7,8-dihydro-5H-pyrido[3′,4′:4,5]pyrrolo[2,3-c]pyridazin-6(9H)-yl)cyclohexyl)methyl)piperazine-1-carboxylate. To a solution of (3R)-12-[2-(methoxymethoxy)phenyl]-3-methyl-4,8,10,11-tetrazatricyclo[7.4.0.02,7]trideca-1(9),2(7),10,12-tetraene (500 mg, 1.54 mmol, Intermediate LE) in THF (10 mL), DMSO (2 mL) was added AcOH (278 mg, 4.62 mmol, 264 uL), tert-butyl 4-[(4-oxocyclohexyl)methyl]piperazine-1-carboxylate (1.37 g, 4.62 mmol, Intermediate NA) at 25° C. for 1.5 hrs. Next, NaBH(OAc)₃ (980 mg, 4.62 mmol) was added at 0° C. Then the mixture was stirred at 25° C. for 12.5 hrs. On completion, the reaction mixture was concentrated under reduced pressure to give the residue. The residue was purified by prep-HPLC (FA) to give the title compound (756 mg, 69% yield) as a yellow solid. LC-MS (ESI⁺) m/z 605 (M+H)⁺.

Step 2-(S)-2-(5-methyl-6-(4-(piperazin-1-ylmethyl)cyclohexyl)-6,7,8,9-tetrahydro-5H-pyrido[3′,4′:4,5]pyrrolo[2,3-c]pyridazin-3-yl)phenol. A solution of tert-butyl 4-[[4-[(3R)-12-[2-(methoxymethoxy)phenyl]-3-methyl-4,8,10,11-tetrazatricyclo[7.4.0.02,7]trideca-1(9),2(7),10,12-tetraen-4-yl]cyclohexyl]methyl]piperazine-1-carboxylate (100 mg, 2 umol) in HCl/dioxane (1 ml) was stirred at 25° C. for 2 hours. On completion, the reaction mixture was concentrated under reduced pressure to give the title compound (100 mg) as a yellow solid. LC-MS (ESI⁺) m/z 461.3 (M+H)⁺.

### N—((S)-1-((2S,4R)-2-((4-ethynylbenzyl)carbamoyl)-4-hydroxypyrrolidin-1-yl)-3,3-dimethyl-1-oxobutan-2-yl)piperidine-4-carboxamide (Intermediate MA)

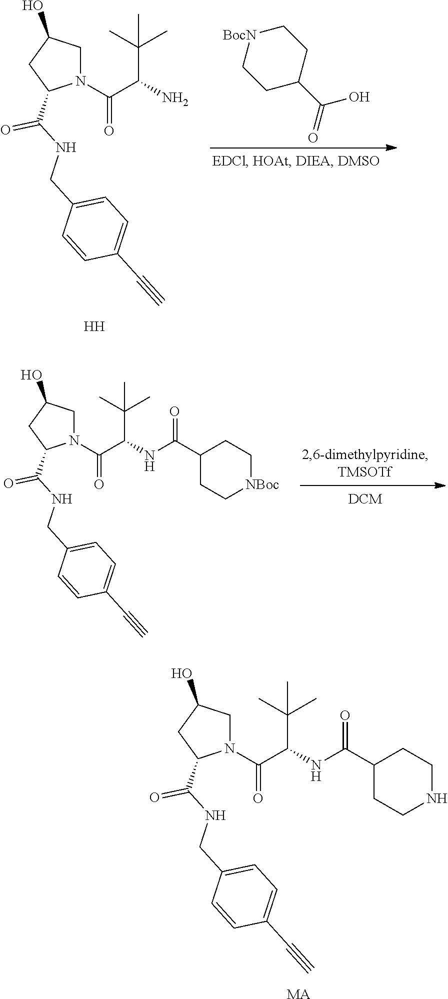

Step 1-Tert-butyl 4-(((S)-1-((2S,4R)-2-((4-ethynylbenzyl)carbamoyl)-4-hydroxypyrrolidin-1-yl)-3,3-dimethyl-1-oxobutan-2-yl)carbamoyl)piperidine-1-carboxylate. To a solution of 1-(tert-butoxycarbonyl)piperidine-4-carboxylic acid (500 mg, 1.40 mmol) in DMSO (5 mL) was added HOAt (286 mg, 2.10 mmol), EDCI (402 mg, 2.10 mmol), DIEA (723 mg, 5.60 mmol) and (2S,4R)-1-((S)-2-amino-3,3-dimethylbutanoyl)-N-(4-ethynylbenzyl)-4-hydroxypyrrolidine-2-carboxamide (353 mg, 1.54 mmol, Intermediate HH). The mixture was then stirred at 25° C. for 2 hrs. On completion, the reaction mixture was concentrated in vacuo to give a residue. The crude product was purified by column chromatography (SiO₂, Dichloromethane: Methanol=10/1) to give the title compound (682 mg, 84% yield) as a yellow solid. LC-MS (ESI⁺) m/z 569.4 (M+H)⁺.

Step 2-N—((S)-1-((2S,4R)-2-((4-ethynylbenzyl)carbamoyl)-4-hydroxypyrrolidin-1-yl)-3,3-dimethyl-1-oxobutan-2-yl)piperidine-4-carboxamide. To a mixture of tert-butyl 4-(((S)-1-((2S,4R)-2-((4-ethynylbenzyl)carbamoyl)-4-hydroxypyrrolidin-1-yl)-3,3-dimethyl-1-oxobutan-2-yl)carbamoyl)piperidine-1-carboxylate (632 mg, 1.11 mmol) in DCM (6 mL) was added TMSOTf (741 mg, 3.33 mmol) and 2,6-dimethylpyridine (179 mg, 1.67 mmol,). The mixture was stirred at 0° C. for 0.5 hour. On completion, the reaction mixture was concentrated in vacuo to give a residue. The crude product was purified by pre-HPLC (0.1% FA condition) to give the title compound (378 mg, 64% yield, FA) as a solid. LC-MS (ESI⁺) m/z 469.5 (M+H)⁺.

### (S)-4-(3-(2-(methoxymethoxy)phenyl)-5-methyl-7,8-dihydro-5H-pyrido[3′,4′:4,5]pyrrolo [2,3-c]pyridazin-6(9H)-yl)cyclohexanecarbaldehyde (Intermediate MB)

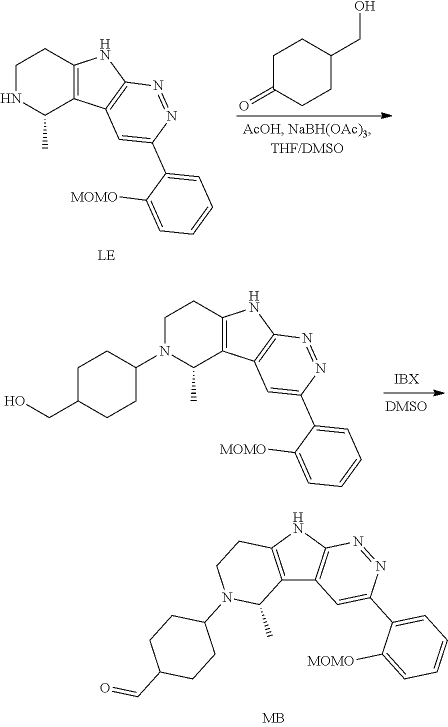

Step 1-(S)-(4-(3-(2-(methoxymethoxy)phenyl)-5-methyl-7,8-dihydro-5H-pyrido [3′,4′:4,5]pyrrolo[2,3-c]pyridazin-6(9H)-yl)cyclohexyl)methanol. To a solution of (S)-3-(2-(methoxymethoxy)phenyl)-5-methyl-6,7,8,9-tetrahydro-5H-pyrido[3′,4′:4,5]pyrrolo[2,3-c]pyridazine (500 mg, 1.54 mmol, Intermediate LE) in THF (5 mL) and DMSO (3 mL) was added AcOH (370 mg, 6.17 mmol) and 4-(hydroxymethyl)cyclohexanone (395 mg, 3.08 mmol) at 25° C. and the mixture was stirred for 2 hours at 40° C. Then NaBH(OAc)₃ (980 mg, 4.62 mmol) was added at 0° C., then the mixture was stirred at 25° C. for 3 hrs. On completion, the reaction mixture was concentrated in vacuo to give a residue. The residue was purified by pre-HPLC (0.1% FA condition) to give the title compound (600 mg, 76% yield, FA) as a yellow solid. LC-MS (ESI⁺) m/z 437.2 (M+H)⁺.

Step 2-(S)-4-(3-(2-(methoxymethoxy)phenyl)-5-methyl-7,8-dihydro-5H-pyrido[3′,4′:4,5]pyrrolo [2,3-c]pyridazin-6(9H)-yl)cyclohexanecarbaldehyde. To a solution of (S)-(4-(3-(2-(methoxymethoxy)phenyl)-5-methyl-7,8-dihydro-5H-pyrido[3′,4′:4,5]pyrrolo[2,3-c]pyridazin-6(9H)-yl)cyclohexyl)methanol (940 mg, 2.2 mmol) in DMSO (25 mL) was added IBX (1.21 g, 4.31 mmol) at 0° C. The reaction was then stirred at 25° C. for 0.5 hr. On completion, the reaction mixture was concentrated in vacuo to give a residue. The crude product was purified by column chromatography (SiO₂, DCM/MEOH=60/1 to 10/1) to give the title compound (359 mg, 19% yield) as a yellow solid. LC-MS (ESI⁺) m/z 435.3 (M+H)⁺.

### Tert-butyl 6-(4-oxopiperidin-1-yl)-2-azaspiro[3.3]heptane-2-carboxylate (Intermediate MC)

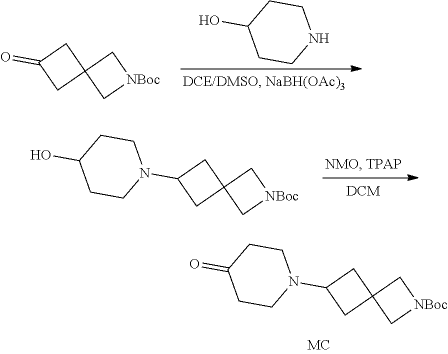

Step 1-(Tert-butyl 6-(4-hydroxypiperidin-1-yl)-2-azaspiro[3.3]heptane-2-carboxylate. To a solution of tert-butyl 6-oxo-2-azaspiro[3.3]heptane-2-carboxylate (10 g, 50 mmol, CAS #1147557-97-8) in DCE (100 mL) and DMSO (15 mL) was added AcOH (5.69 g, 94.6 mmol) and piperidin-4-ol (7.18 g, 71.0 mmol, CAS #5382-16-1) and the mixture was stirred for 1 hr at rt. Then NaBH(OAc)₃ (15.0 g, 71.0 mmol) was added at 0° C. The mixture was then stirred at 25° C. for 2 hours. On completion, the reaction mixture was quenched with H₂O (20 mL), and then extracted with EA (200 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=1/1 to DCM: MeOH=50:1) to give the title compound (11.6 g, 80% yield) as a white solid. LC-MS (ESI⁺) m/z 297.3 (M+H)⁺.

Step 2-Tert-butyl 6-(4-oxopiperidin-1-yl)-2-azaspiro[3.3]heptane-2-carboxylate. To a solution of tert-butyl 6-(4-hydroxy-1-piperidyl)-2-azaspiro[3.3]heptane-2-carboxylate (11.6 g, 39.1 mmol) in DCM (120 mL) was added 4 Å molecular sieves (12 g), then oxido(trioxo)ruthenium;tetrapropylammonium (687 mg, 1.96 mmol) and 4-methyl-4-oxido-morpholin-4-ium (6.88 g, 58.7 mmol) was added at 0° C. The mixture was then stirred at 25° C. for 3 hrs. On completion, the reaction mixture was quenched with H₂O (20 mL) and extracted with EA (200 mL×3). The combined organic layers were washed with brine (100 mL), dried over Na₂SO₄, filtered and concentrated under reduced pressure to give a residue. The residue was purified by column chromatography (SiO₂, DCM: Ethyl acetate=50:1 to 0:1) to give the title compound (10 g, 73% yield) as a white solid. LC-MS (ESI⁺) m/z 313.2 (M+18+H)⁺.

### (R)-2-(6-(1-(2-azaspiro[3.3]heptan-6-yl)piperidin-4-yl)-5-methyl-6,7,8,9-tetrahydro-5H-pyrido[3′,4′:4,5]pyrrolo[2,3-c]pyridazin-3-yl)phenol (Intermediate MD)

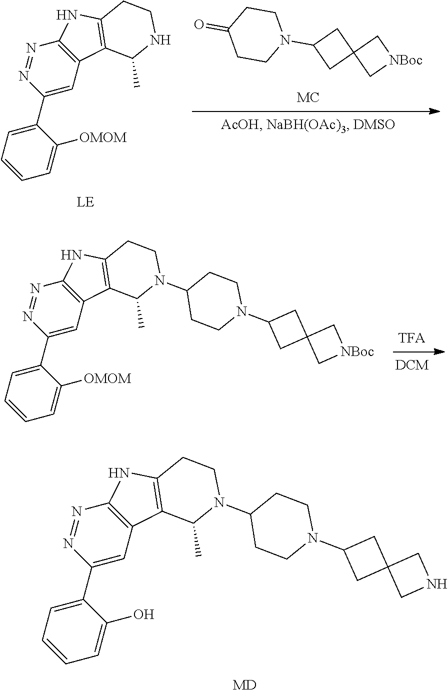

Step 1-(R)-tert-butyl 6-(4-(3-(2-(methoxymethoxy)phenyl)-5-methyl-7,8-dihydro-5H-pyrido[3′,4′:4,5]pyrrolo[2,3-c]pyridazin-6(9H)-yl)piperidin-1-yl)-2-azaspiro[3.3]heptane-2-carboxylate. To a solution of (3R)-12-[2-(methoxymethoxy)phenyl]-3-methyl-4,8,10,11-tetrazatricyclo[7.4.0.0^{2,7}]trideca-1(9),2(7),10,12-tetraene (1 g, 3 mmol, Intermediate LE) in DMSO (10 mL) was added AcOH (370 mg, 6.17 mmol) and tert-butyl 6-(4-oxo-1-piperidyl)-2-azaspiro[3.3]heptane-2-carboxylate (2.72 g, 9.25 mmol, Intermediate MC). The mixture was stirred at 40° C. for 2 hrs. Then NaBH(OAc)₃ (980 mg, 4.62 mmol) was added at 0° C. The mixture was stirred at 25° C. for 13 hrs. On completion, the reaction mixture was diluted with H₂O (10 mL) and extracted with EA (100 mL×3). The combined organic layers were washed with brine (20 mL×2), dried over Na₂SO₄, filtered and concentrated under reduced pressure to give a residue. The residue was purified by column chromatography (SiO₂, DCM: MeOH=50:1 to 10:1) to give the title compound (1.7 g, 77% yield) as a yellow solid. LC-MS (ESI⁺) m/z 603.5 (M+H)⁺.

Step 2-(R)-2-(6-(1-(2-azaspiro[3.3]heptan-6-yl)piperidin-4-yl)-5-methyl-6,7,8,9-tetrahydro-5H-pyrido[3′,4′:4,5]pyrrolo[2,3-c]pyridazin-3-yl)phenol. To a solution of tert-butyl 6-[4-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]-1-piperidyl]-2-azaspiro[3.3]heptane-2-carboxylate (1.6 g, 2.7 mmol) in DCM (12 mL) was added TFA (22.7 g, 199 mmol). The mixture was stirred at 25° C. for 12 hrs. On completion, the reaction mixture was filtered and concentrated under reduced pressure to give a residue. The residue was purified by prep-HPLC (FA condition) to give the title compound (1.3 g, 90% yield, FA) as a yellow solid. LC-MS (ESI⁺) m/z 459.4 (M+H)⁺.

### (R)-2-(6-(1-([2,6′-bi2,2′-diazaspiro[3.3]heptan]-6-yl)piperidin-4-yl)-5-methyl-6,7,8,9-tetrahydro-5H-pyrido[3′,4′:4,5]pyrrolo[2,3-c]pyridazin-3-yl)phenol (Intermediate ME)

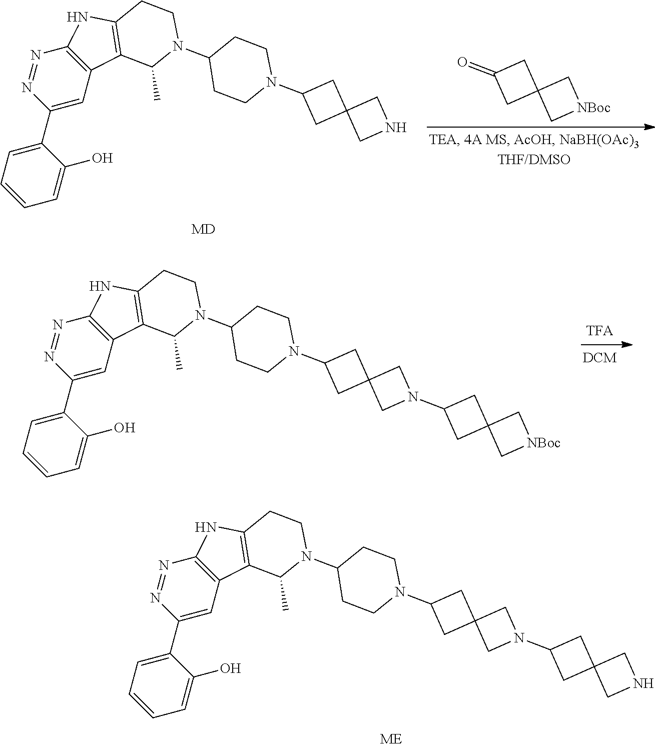

Step 1-(R)-tert-butyl 6-(4-(3-(2-hydroxyphenyl)-5-methyl-7,8-dihydro-5H-pyrido[3′,4′:4,5]pyrrolo[2,3-c]pyridazin-6(9H)-yl)piperidin-1-yl)-2,2′-diaza[2,6′-bispiro[3.3]heptane]-2′-carboxylate. To a solution of 2-[(3R)-4-[1-(2-azaspiro[3.3]heptan-6-yl)-4-piperidyl]-3-methyl-4,8,10,11-tetrazatricyclo[7.4.0.0^{2,7}]trideca-1(9),2(7),10,12-tetraen-12-yl]phenol (500 mg, 1.09 mmol, Intermediate MD) in THF (5 mL) and DMSO (1 mL) was added 4 Å molecular sieves (30 mg) and TEA (220 mg, 2.2 mmol). The mixture was stirred at 0° C. for 1 hr. Then tert-butyl 6-oxo-2-azaspiro[3.3]heptane-2-carboxylate (253 mg, 1.20 mmol) and AcOH (130 mg, 2.18 mmol) was added at 0° C. and the mixture was stirred for another 2 hrs. Then NaBH(OAc)₃ (693 mg, 3.27 mmol) was added at 0° C. and the mixture was stirred for 2 hrs. On completion, the reaction mixture was quenched with H₂O (0.5 mL) and then filtered and concentrated under reduced pressure to give a residue. The residue was purified by prep-HPLC (FA condition) to give the title compound (170 mg, 21% yield, FA) as a yellow solid. LC-MS (ESI⁺) m/z 654.5 (M+H)⁺.

Step 2-(R)-2-(6-(1-([2,6′-bi2,2′-diazaspiro[3.3]heptan]-6-yl)piperidin-4-yl)-5-methyl-6,7,8,9-tetrahydro-5H-pyrido[3′,4′:4,5]pyrrolo[2,3-c]pyridazin-3-yl)phenol. To a solution of tert-butyl 6-[6-[4-[(3R)-12-(2-hydroxyphenyl)-3-methyl-4,8,10,11-tetrazatricyclo[7.4.0.0^{2,7}]trideca-1(9),2(7),10,12-tetraen-4-yl]-1-piperidyl]-2-azaspiro[3.3]heptan-2-yl]-2-azaspiro[3.3]heptane-2-carboxylate (150 mg, 230 umol) in DCM (1.5 mL) was added TFA (1.54 g, 13.5 mmol). The mixture was then stirred at 25° C. for 1 hr. On completion, the reaction mixture was filtered and concentrated under reduced pressure to give a residue. The residue was purified by prep-HPLC (FA condition) to give the title compound (50 mg, 87% yield, FA) as a yellow solid. LC-MS (ESI⁺) m/z 554.3 (M+H)⁺.

### N—((S)-1-((2S,4R)-2-((4-ethynylbenzyl)carbamoyl)-4-hydroxypyrrolidin-1-yl)-3,3-dimethyl-1-oxobutan-2-yl)-4-oxopiperidine-1-carboxamide (Intermediate MF)

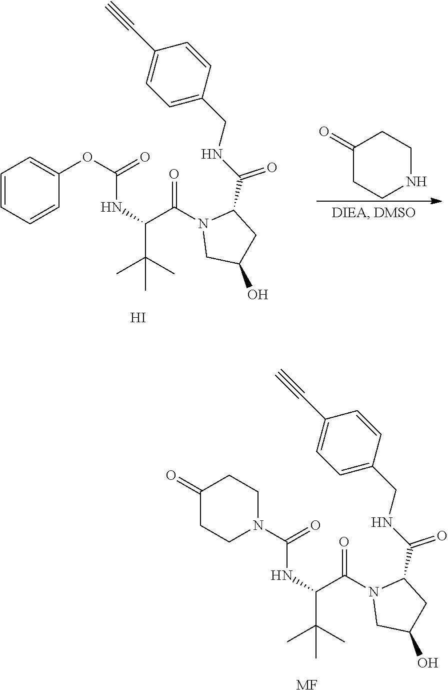

To a solution of phenyl ((S)-1-((2S,4R)-2-((4-ethynylbenzyl)carbamoyl)-4-hydroxypyrrolidi-1-yl)-3,3-dimethyl-1-oxobutan-2-yl)carbamate (1 g, 2 mmol, Intermediate HI) in DMSO (10 mL) was added DIEA (1.08 g, 8.36 mmol) and piperidin-4-one (249 mg, 2.51 mmol) and the mixture was stirred at 80° C. for 12 hrs. On completion, the reaction mixture was concentrated in vacuo to give a residue. The residue was purified by reversed-phase HPLC(0.1% FA condition) to give the title compound (800 mg, 67% yield, FA) as a yellow solid. LC-MS (ESI⁺) m/z 483.5 (M+H)⁺.

### (R)-2-(6-([2,6′-bi2,2′-diazaspiro[3.3]heptan]-6-yl)-5-methyl-6,7,8,9-tetrahydro-5H-pyrido[3′,4′:4,5]pyrrolo[2,3-c]pyridazin-3-yl)phenol (Intermediate MG)

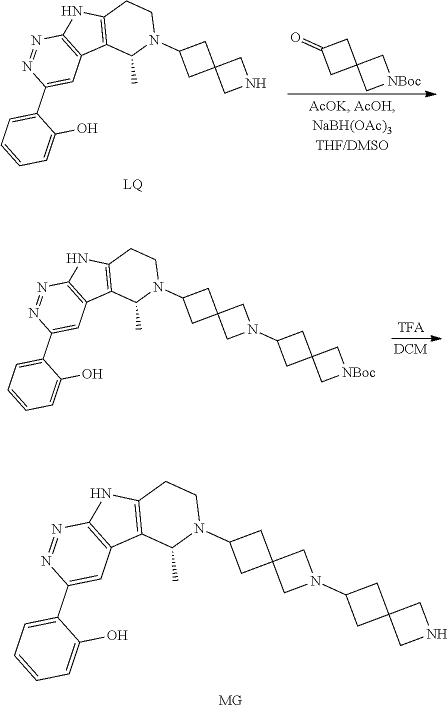 [SMILES: C[C@@H]1c2c([nH]c3nnc(-c4ccccc4O)cc23)CCN1C1CC2(CNC2)C1 ; CC(C)(C)OC(=O)N1CC2(CC(=O)C2)C1 ; C[C@@H]1c2c([nH]c3nnc(-c4ccccc4O)cc23)CCN1C1CC2(C1)CN(C1CC3(C1)CN(C(=O)OC(C)(C)C)C3)C2 ; C[C@@H]1c2c([nH]c3nnc(-c4ccccc4O)cc23)CCN1C1CC2(C1)CN(C1CC3(CNC3)C1)C2]

Step 1-(R)-tert-butyl 6-(3-(2-hydroxyphenyl)-5-methyl-7,8-dihydro-5H-pyrido[3′,4′:4,5] pyrrolo[2,3-c]pyridazin-6(9H)-yl)-2,2′-diaza[2,6′-bispiro[3.3]heptane]-2′-carboxylate. To a solution of (R)-2-(5-methyl-6-(2-azaspiro[3.3]heptan-6-yl)-6,7,8,9-tetrahydro-5H-pyrido[3′,4′:4,5]pyrrolo[2,3-c]pyridazin-3-yl)phenol (190 mg, 510 umol, Intermediate LQ) in DMSO (1 mL) and THF (1 mL) was added AcOK (149 mg, 1.52 mmol) at 0° C. and the mixture was stirred for 1 hr. Then AcOH (122 mg, 2.02 mmol) and tert-butyl 6-oxo-2-azaspiro[3.3]heptane-2-carboxylate (214 mg, 1.01 mmol) was added at 0° C. and the mixture was stirred for 1 hr. Finally, NaBH(OAc)₃ (322 mg, 1.52 mmol) was added at 0° C. and the mixture was stirred for 3 hrs. On completion, the reaction mixture was concentrated in vacuo to give a residue. The crude product was purified by column chromatography (SiO₂, Petroleum ether/Ethyl acetate=20/1) to give the title compound (108 mg, 36% yield) as a yellow solid. LC-MS (ESI⁺) m/z 571.5 (M+H)⁺.

Step 2-(R)-2-(6-([2,6′-bi2,2′-diazaspiro[3.3]heptan]-6-yl)-5-methyl-6,7,8,9-tetrahydro-5H-pyrido[3′,4′:4,5]pyrrolo[2,3-c]pyridazin-3-yl)phenol. To a mixture of (R)-tert-butyl 6-(3-(2-hydroxyphenyl)-5-methyl-7,8-dihydro-5H-pyrido[3′,4′:4,5]pyrrolo[2,3-c]pyridazin-6(9H)-yl)-2,2′-diaza[2,6′-bispiro[3.3]heptane]-2′-carboxylate (108 mg, 189.23 umol) in DCM (1.5 mL) was added TFA (462 mg, 4.05 mmol. The mixture was then stirred at 25° C. for 0.5 hr. On completion, the reaction mixture was concentrated in vacuo to give a residue. The crude product was purified by reversed-phase HPLC(0.1% FA condition) to give the title compound (60 mg, 59% yield, FA) as a yellow solid. LC-MS (ESI⁺) m/z 471.3 (M+H)⁺.

### (2S,4R)-1-((S)-3,3-dimethyl-2-((phenoxycarbonyl)amino)butanoyl)-4-(phosphonooxy)pyrrolidine-2-carboxylic acid (Intermediate MH)

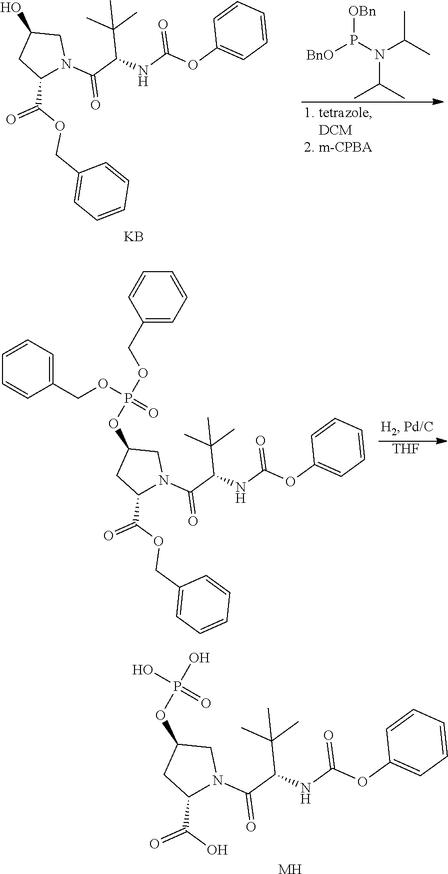

Step 1-(2S,4R)-benzyl 4-((bis(benzyloxy)phosphoryl)oxy)-1-((S)-3,3-dimethyl-2-((phenoxycarbonyl)amino)butanoyl)pyrrolidine-2-carboxylate. To a solution of benzyl (2S,4R)-1-[(2S)-3,3-dimethyl-2-(phenoxycarbonylamino)butanoyl]-4-hydroxy-pyrrolidine-2-carboxylate (2 g, 4 mmol, Intermediate KB) in DCM (50 mL) was added 2H-tetrazole (0.45 M, 29.3 mL). Then N-dibenzyloxyphosphanyl-N-isopropyl-propan-2-amine (3.04 g, 8.80 mmol, CAS #108549-23-1) was added and the mixture was stirred at 25° C. for 2 hrs. Next, m-CPBA (2.85 g, 13.2 mmol) was added at 0° C. and the mixture was stirred at 25° C. for another 2 hrs. On completion, the reaction mixture was quenched by saturated sodium sulfite aqueous solution (100 mL) and saturated sodium bicarbonate aqueous solution (100 mL), then the mixture was stirred for 1 hr. After this, the mixture was extracted with dichloromethane (3×100 mL). The extracts were washed with brine (100 mL) and dried over anhydrous sodium sulfate, filtered and concentrated in vacuo to get the crude residue. The crude was purified by silica gel chromatography (petroleum ether:ethyl acetate=10:1 to 1:1) to give the title compound (3 g, 81% yield) as a colorless oil. LC-MS (ESI+) m/z 715.2 (M+H)⁺.

Step 2-(2S,4R)-1-((S)-3,3-dimethyl-2-((phenoxycarbonyl)amino)butanoyl)-4-(phosphonooxy)pyrrolidine-2-carboxylic acid. To a solution of benzyl (2S,4R)-4-dibenzyloxyphosphoryloxy-1-[(2S)-3,3-dimethyl-2-(phenoxycarbonylamino)butanoyl]pyrrolidine-2-carboxylate (1.5 g, 2.1 mmol) in THF (15 mL) was added Pd/C (10 wt %, 1.5 g) under N₂ atmosphere. The suspension was degassed and purged with H₂ three times. The mixture was then stirred under H₂ (15 Psi) at 25° C. for 12 hrs. On completion, the reaction mixture was filtered and concentrated under reduced pressure to give the title compound (1 g) as a black solid. LC-MS (ESI+) m/z 445.1 (M+H)⁺.

### Phenyl ((S)-1-((2S,4R)-2-((4-ethynyl-2-fluorobenzyl)carbamoyl)-4-(phosphonooxy)pyrrolidin-1-yl)-3,3-dimethyl-1-oxobutan-2-yl)carbamate (Intermediate MI)

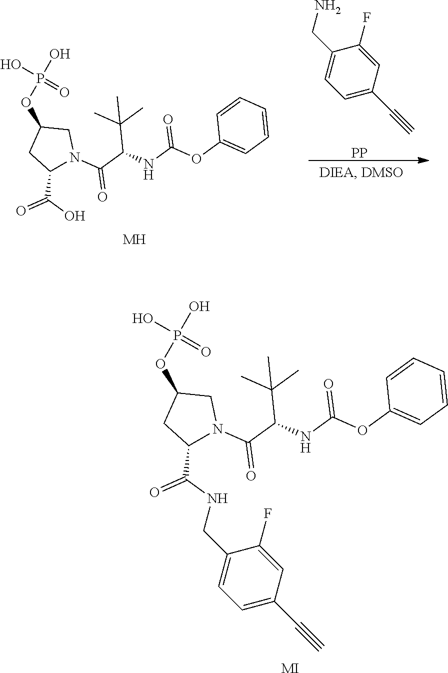

To a solution of (2S,4R)-1-[(2S)-3,3-dimethyl-2-(phenoxycarbonylamino)butanoyl]-4-phosphonooxy-pyrrolidine-2-carboxylic acid (500 mg, 1.13 mmol, Intermediate MH) and (4-ethynyl-2-fluoro-phenyl)methanamine (168 mg, 1.13 mmol, Intermediate PP) in DMSO (5 mL) was added DIEA (436 mg, 3.38 mmol) and HATU (642 mg, 1.69 mmol). The mixture was stirred at 25° C. for 12 hrs. On completion, the reaction mixture was purified directly by reversed-phase HPLC (0.1% FA condition) to give the title compound (300 mg, 37% yield, FA) as a yellow solid. ¹H NMR (400 MHz, DMSO-d₆) δ=8.72 (s, 1H), 7.85 (d, J=8.4 Hz, 1H), 7.50 (t, J=8.0 Hz, 1H), 7.40-7.28 (m, 3H), 7.24-7.17 (m, 2H), 7.11 (d, J=7.6 Hz, 2H), 4.85 (s, 1H), 4.46 (t, J=8.0 Hz, 1H), 4.36 (d, J=5.6 Hz, 1H), 4.28 (s, 2H), 4.25-4.18 (m, 1H), 3.86-3.76 (m, 2H), 2.36-2.30 (m, 1H), 2.03 (d, J=8.0 Hz, 1H), 1.05-0.96 (m, 9H). LC-MS (ESI+) m/z 576.4 (M+H)⁺.

### (2S,4R)-1-[(2S)-2-amino-3,3-dimethyl-butanoyl]-N-[(4-ethynyl-2-fluoro-phenyl)methyl]-4-hydroxy-pyrrolidine-2-carboxamide (Intermediate MJ)

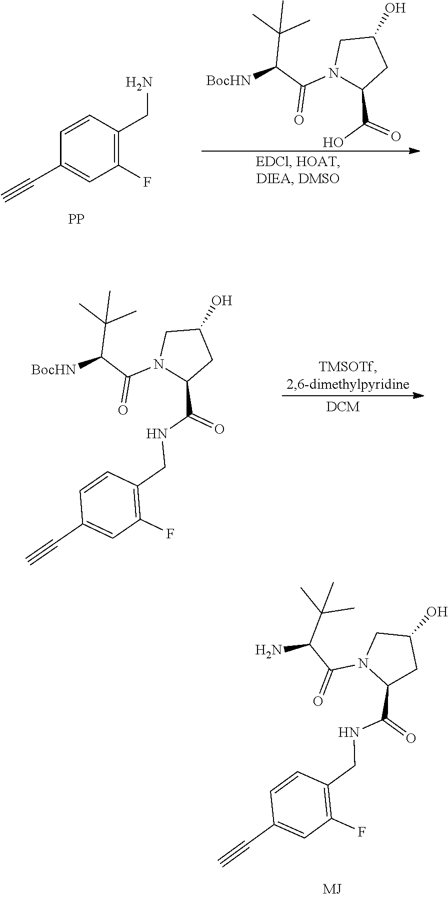

Step 1-Tert-butyl ((S)-1-((2S,4R)-2-((4-ethynyl-2-fluorobenzyl)carbamoyl)-4-hydroxypyrrolidin-1-yl)-3,3-dimethyl-1-oxobutan-2-yl)carbamate. To a solution of (2S,4R)-1-[(2S)-2-(tert-butoxycarbonylamino)-3,3-dimethyl-butanoyl]-4-hydroxy-pyrrolidine-2-carboxylic acid (7 g, 20.3 mmol, CAS #630421-46-4) in DMSO (300 mL) was added EDCI (5.84 g, 30.5 mmol) and HOAt (4.15 g, 30.5 mmol, 4.26 mL), DIEA (15.8 g, 122.0 mmol, 21.2 mL) and (4-ethynyl-2-fluoro-phenyl)methanamine (9.10 g, 61.0 mmol, Intermediate PP). The mixture was then stirred at 25° C. for 12 hrs. On completion, the reaction mixture was diluted with water (70 mL) and extracted with DCM (100 mL×3). The combined organic layers were washed with aqueous NaCl (100 mL×3), dried over Na₂SO₄, filtered and concentrated under reduced pressure to give a residue. The residue was purified by column chromatography (SiO₂, DCM/MeOH=50/1 to 10/1) to give the title compound (6.5 g, 65% yield) as a yellow oil. LC-MS (ESI⁺) m/z 476.1 (M+H)⁺.

Step 3-(2S,4R)-1-((S)-2-amino-3,3-dimethylbutanoyl)-N-(4-ethynyl-2-fluorobenzyl)-4-hydroxypyrrolidine-2-carboxamide. To a solution of tert-butyl N-[(1S)-1-[(2S,4R)-2-[(4-ethynyl-2-fluoro-phenyl)methylcarbamoyl]-4-hydroxy-pyrrolidine-1-carbonyl]-2,2-dimethyl-propyl]carbamate (2 g, 4.21 mmol) in DCM (20 mL) was added TMSOTf (2.80 g, 12.6 mmol, 2.28 mL) and 2,6-dimethyl(115N)pyridine (1.36 g, 12.6 mmol). The mixture was then stirred at 25° C. for 2 hrs. On completion, the reaction concentrated under reduced pressure to give the residue. The residue was purified by prep-HPLC (FA condition) to give the title compound (0.5 g, 23% yield) as a yellow solid. LC-MS (ESI⁺) m/z 376.1 (M+H)⁺.

### 2-[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]spiro[3.5]nonane-7-carboxylic acid (Intermediate MK) and (S)-2-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.5]nonane-7-carboxylic acid (Intermediate IN)

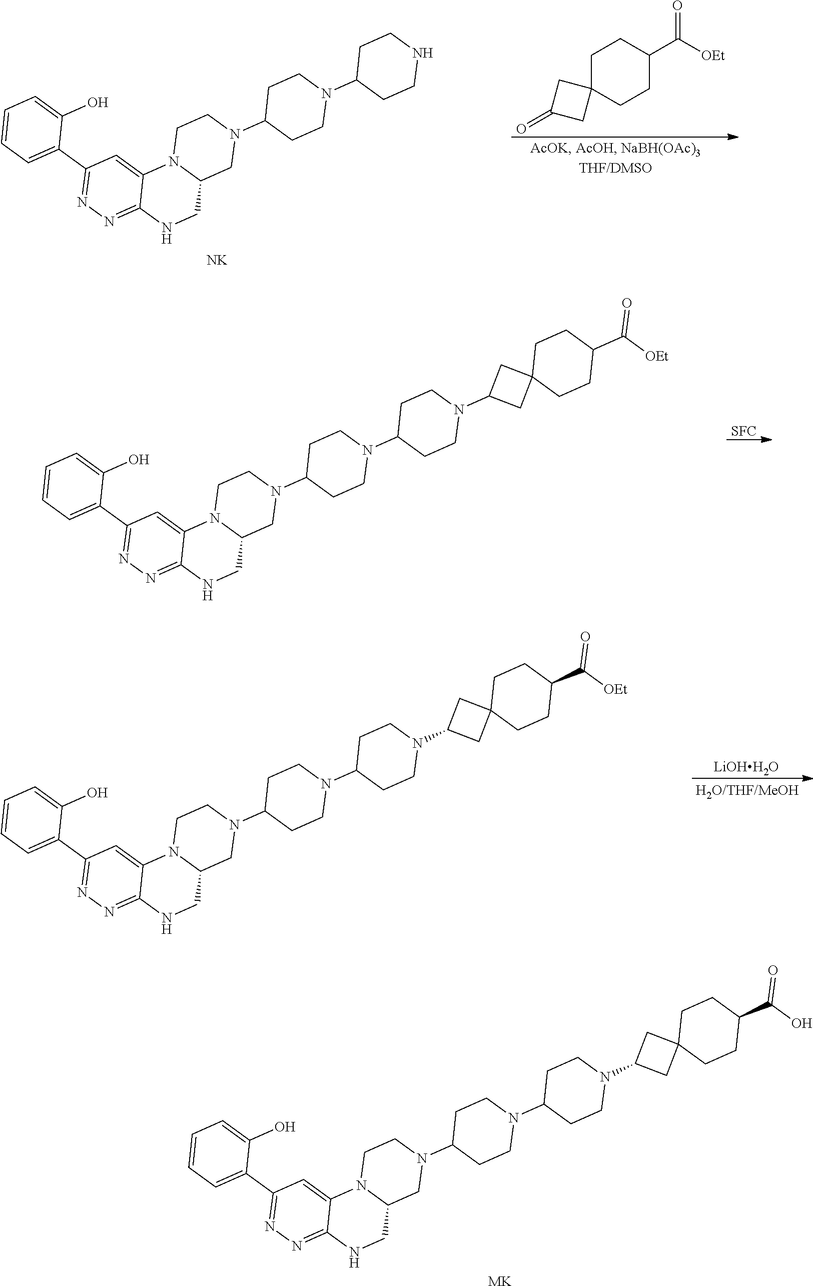

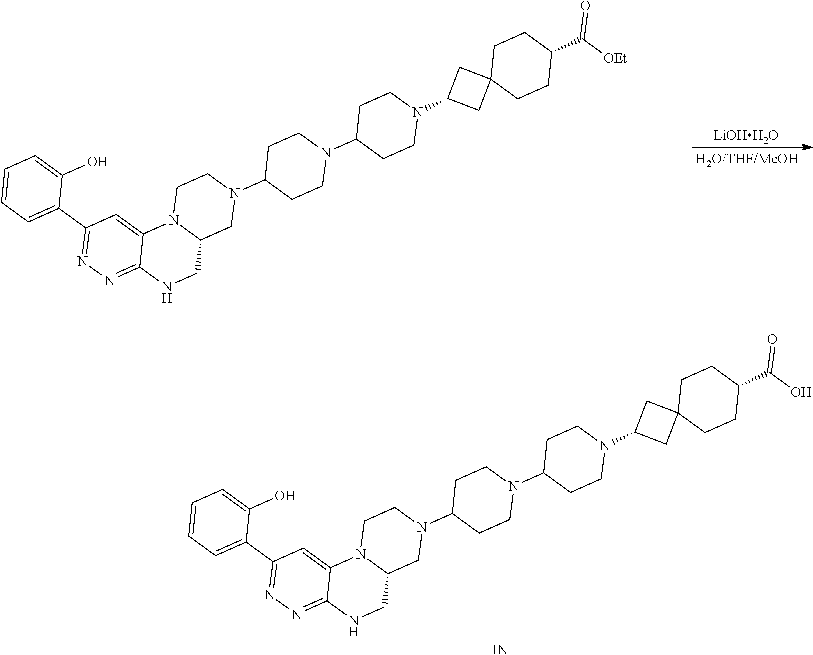 [SMILES: CCOC(=O)[C@H]1CCC2(CC1)C[C@H](N1CCC(N3CCC(N4CCN5c6cc(-c7ccccc7O)nnc6NC[C@H]5C4)CC3)CC1)C2 ; "LiOH·H2O" ; O=C(O)[C@H]1CCC2(CC1)C[C@H](N1CCC(N3CCC(N4CCN5c6cc(-c7ccccc7O)nnc6NC[C@H]5C4)CC3)CC1)C2 ; "IN"]

Step 1-(S)-ethyl 2-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.5]nonane-7-carboxylate. To a solution of 2-[(10S)-12-[1-(4-piperidyl)-4-piperidyl]-1,5,6,8,12-pentazatricyclo[8.4.0.02,7]tetradeca-2,4,6-trien-4-yl]phenol(1.7 g, 3.8 mmol, Intermediate NK) in THF (24 mL) and DMSO (5 mL) was added AcOK (1.11 g, 11.3 mmol) and the mixture was stirred for 0.5 hrs. Then AcOH (681 mg, 11.3 mmol, 648 uL) and ethyl 2-oxospiro[3.5]nonane-7-carboxylate (1.99 g, 9.45 mmol) was added and the mixture was stirred for another 1.5 hrs. At last, NaBH(OAc)₃ (2.40 g, 11.3 mmol) was added at 0° C. and the mixture was stirred at 25° C. for 12 hrs. On completion, the reaction mixture was concentrated under reduced pressure to give the residue. The residue was purified by prep-HPLC (FA condition) to give the title compound (1.55 g, 59% yield) as a white solid. LC-MS (ESI⁺) m z 644.3 (M+H)⁺.

Step 2-Ethyl 2-(4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)spiro[3.5]nonane-7-carboxylate and ethyl 2-(4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)spiro[3.5]nonane-7-carboxylate. (S)-ethyl 2-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.5]nonane-7-carboxylate was separated by SFC (column: DAICEL CHIRALPAK IA (250 mm×30 mm, 10 um); mobile phase: [0.1% NH₃H₂O, EtOH]; B %: 75%-75%, 10; 180 min) to give ethyl 2-(4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)spiro[3.5]nonane-7-carboxylate (720 mg, 41% yield) as a white solid (peak 1, LC-MS (ESI⁺) m/z 644.7 (M+H)⁺) and ethyl 2-(4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)spiro[3.5]nonane-7-carboxylate (530 mg, 34% yield) as a white solid (peak 2, LC-MS (ESI⁺) m/z 644.5 (M+H)⁺).

Step 3-(S)-2-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.5]nonane-7-carboxylic acid. To a solution of ethyl 2-[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]spiro[3.5]nonane-7-carboxylate (700 mg, 1.09 mmol, peak 1) in H₂O (5 mL), THF (10 mL) and MeOH (10 mL) was added LiOH·H₂O (273 mg, 6.52 mmol). The mixture was stirred at 25° C. for 2 hrs. On completion, the reaction mixture was concentrated under reduced pressure to give the residue. The residue was purified by prep-HPLC (HCl condition) to give the title compound (530 g, 79% yield) as a red solid. LC-MS (ESI⁺) m z 616.4 (M+H)⁺.

Step 4-(S)-2-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.5]nonane-7-carboxylic acid. To a solution of ethyl 2-[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]spiro[3.5]nonane-7-carboxylate (480 mg, 0.74 mmol) in H₂O (1 mL) THF (1 mL) MeOH (1 mL) was added LiOH·H₂O (188 mg, 4.47 mmol). The mixture was stirred at 25° C. for 2 hrs. On completion, the reaction mixture was concentrated under reduced pressure to give the residue. The residue was purified by prep-HPLC (HCl condition) to give the title compound (330 g, 68% yield) as a red solid. LC-MS (ESI⁺) m/z 616.4 (M+H)⁺.

### Benzyl 4-((4-oxopiperidin-1-yl)methyl)piperidine-1-carboxylate (Intermediate ML)

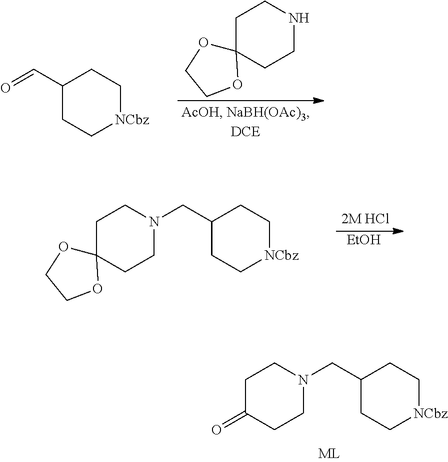 [SMILES: "EtOH" ; O=CC1CCN(C(=O)OCc2ccccc2)CC1 ; C1CC2(CCN1)OCCO2 ; O=C(OCc1ccccc1)N1CCC(CN2CCC3(CC2)OCCO3)CC1 ; O=C1CCN(CC2CCN(C(=O)OCc3ccccc3)CC2)CC1]

Step 1-Benzyl 4-(1,4-dioxa-8-azaspiro[4.5]decan-8-ylmethyl)piperidine-1-carboxylate. To a solution of benzyl 4-formylpiperidine-1-carboxylate (5 g, 20.2 mmol, CAS #138163-08-3) in 1,2-dichloroethane (80 mL) was added 1,4-dioxa-8-azaspiro[4.5]decane (2.90 g, 20.2 mmol, CAS #177-11-7), AcOH (121 mg, 2.02 mmol) and 4 Å molecular sieves (8 g). The mixture was stirred at 25° C. for 2 hrs, and then sodium triacetoxyboranuide (8.57 g, 40.4 mmol) was added dropwise at 0° C. The resulting mixture was then stirred at 25° C. for 6 hrs. The reaction mixture was quenched with sat. NaHCO₃ (60 mL), and then diluted with H₂O (40 mL) extracted with EA (100 mL*3) and filtered. The combined organic layers were washed with brine (100 mL), dried over Na₂SO₄, filtered and concentrated under reduced pressure to give a residue. The residue was purified by column chromatography (SiO₂, DCM: MeOH=10:1) to give title compound (6.20 g, 79% yield) as a yellow solid. LC-MS (ESI⁺) m/z 375.5 (M+H)⁺.

Step 2-Benzyl 4-((4-oxopiperidin-1-yl)methyl)piperidine-1-carboxylate. To a solution of benzyl 4-(1,4-dioxa-8-azaspiro[4.5]decan-8-ylmethyl)piperidine-1-carboxylate (4.2 g, 11.2 mmol) in EtOH (40 mL) was added HCl (2 M, 56 mL). The mixture was stirred at 70° C. for 12 hrs. The reaction mixture was neutralized by addition with 2M NaOH and extracted with EA (60 mL×3). The combined organic layers were washed with brine (80 mL), dried over Na₂SO₄, filtered and concentrated under reduced pressure to give a residue. The residue was purified by column chromatography (SiO₂, DCM: MeOH=10:1) to give the title compound (2.50 g, 62% yield) as a white solid. LC-MS (ESI⁺) m/z 349.2 (M+H+H₂O)⁺

### (R)-2-(5-methyl-6-(1′-(piperidin-4-ylmethyl)-[1,4′-bipiperidin]-4-yl)-6,7,8,9-tetrahydro-5H-pyrido[3′,4′:4,5]pyrrolo[2,3-c]pyridazin-3-yl)phenol (Intermediate MM)

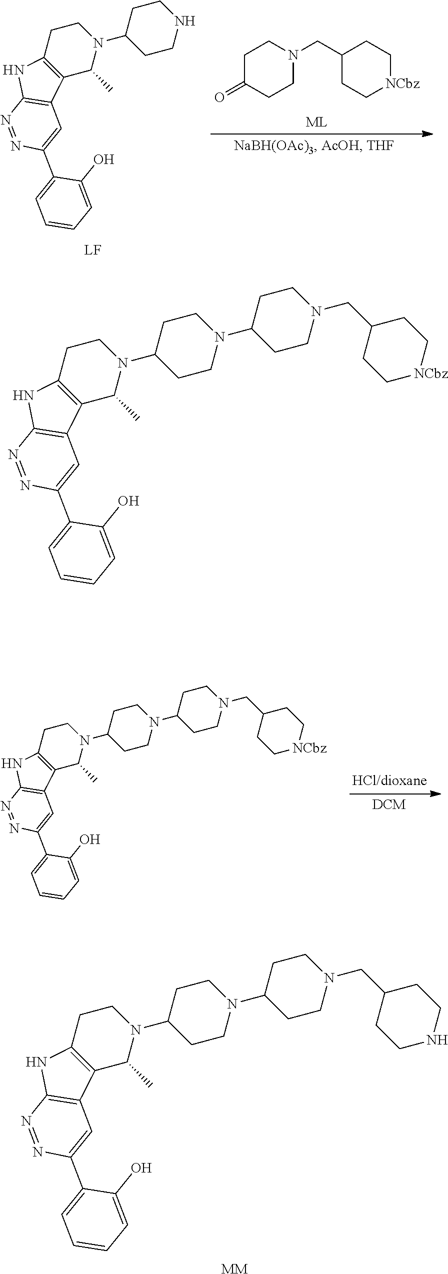

Step 1-(R)-benzyl 4-((4-(3-(2-hydroxyphenyl)-5-methyl-7,8-dihydro-5H-pyrido[3′,4′:4,5]pyrrolo[2,3-c]pyridazin-6(9H)-yl)-[1,4′-bipiperidin]-1′-yl)methyl)piperidine-1-carboxylate. To a solution of benzyl 4-[(4-oxo-1-piperidyl)methyl]piperidine-1-carboxylate (500 mg, 1.51 mmol, Intermediate ML) in THF (10 mL) and DMSO (5 mL) was added dropwise 2-[(3R)-3-methyl-4-(4-piperidyl)-4,8,10,11-tetrazatricyclo[7.4.0.02,7]trideca-1(9),2(7),10,12-tetraen-12-yl]phenol (605.16 mg, 1.51 mmol, HCl salt, Intermediate LF) followed by 4 Å molecular sieves (1 g), AcOK (445 mg, 4.54 mmol) and AcOH (363 mg, 6.05 mmol). The mixture was stirred at 25° C. for 2 hrs, and then sodium triacetoxyboranuide (962 mg, 4.54 mmol) was added dropwise at 0° C. The resulting mixture was stirred at 25° C. for 5 hrs. On completion, the reaction mixture was quenched with sat. NaHCO₃ (20 mL), and then diluted with H₂O (20 mL), extracted with DCM (50 mL×3) and filtered. The combined organic layers were washed with brine (70 mL), dried over Na₂SO₄, filtered and concentrated under reduced pressure to give a residue. The residue was purified by column chromatography (SiO₂, DCM: MeOH=10:1) to give the title compound (950 mg, 24% yield) as a yellow solid. LC-MS (ESI⁺) m/z678.7 (M+H).

Step 2-(R)-2-(5-methyl-6-(1′-(piperidin-4-ylmethyl)-[1,4′-bipiperidin]-4-yl)-6,7,8,9-tetrahydro-5H-pyrido[3′,4′:4,5]pyrrolo[2,3-c]pyridazin-3-yl)phenol. To a solution of benzyl 4-[[4-[4-[(3R)-12-(2-hydroxyphenyl)-3-methyl-4,8,10,11-tetrazatricyclo[7.4.0.02,7]trideca-1(9),2(7),10,12-tetraen-4-yl]-1-piperidyl]-1-piperidyl]methyl]piperidine-1-carboxylate (390 mg, 575 umol) in DCM (4 mL) was added HCl/dioxane (4 M, 12 mL). The mixture was then stirred at 25° C. for 12 hrs. On completion, the reaction mixture was concentrated under reduced pressure to give a residue. The crude product was purified by reversed-phase HPLC(0.1% HCl condition) to give title compound (200 mg, 48% yield, HCl salt) as a yellow solid. LC-MS (ESI⁺) m/z544.2 (M+H)⁺.

### 1-(4-Ethynylphenyl)cyclopropanamine (Intermediate MO)

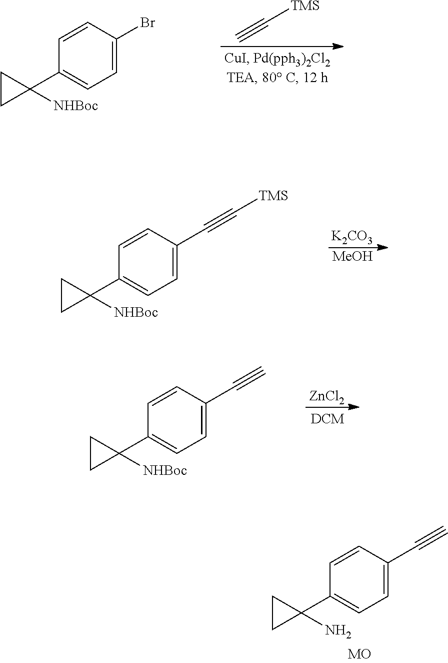

Step 1-Tert-butyl (1-(4-((trimethylsilyl)ethynyl)phenyl)cyclopropyl)carbamate. To a solution of tert-butyl N-[1-(4-bromophenyl)cyclopropyl]carbamate (20 g, 60 mmol, CAS #360773-84-8) in TEA (250 mL) was added ethynyl(trimethyl)silane (37.8 g, 384 mmol, 53.2 mL), Pd(PPh₃)₂Cl₂ (2.25 g, 3.20 mmol) and CuI (1.22 g, 6.41 mmol). The mixture was stirred at 80° C. for 12 hr. On completion, the reaction mixture was partitioned between DCM (300 mL) and H₂O (200 mL). The organic phase was separated, washed with NaCl 210 mL (70 mL×3), dried over Na₂SO₄, filtered and concentrated under reduced pressure to give a residue. The residue was purified by column chromatography (SiO₂, Petroleum ether/Ethyl acetate=100/1 to 20/1) to give the title compound (24.5 g, 93% yield) as a brown solid. ¹H NMR (400 MHz, DMSO-d6) δ=7.72 (s, 1H), 7.35 (br d, J=8.1 Hz, 2H), 7.09 (br d, J=8.1 Hz, 2H), 1.38 (s, 7H), 1.24 (br s, 2H), 1.14 (br d, J=6.1 Hz, 4H), 0.22 (s, 9H); LC-MS (ESI⁺) m/z 274.2 (M+H−56)⁺.

Step 2-Tert-butyl (1-(4-ethynylphenyl)cyclopropyl)carbamate. To a solution of tert-butyl N-[1-[4-(2-trimethylsilylethynyl)phenyl]cyclopropyl]carbamate (24.5 g, 74.3 mmol) in MeOH (350 mL) was added K₂CO₃ (20.6 g, 149 mmol). The mixture was stirred at 25° C. for 0.5 hr. On completion, the reaction mixture was partitioned between EA (400 mL) and H₂O (200 mL). The organic phase was separated, washed with NaCl (100 mL×3), dried over Na₂SO₄, filtered and concentrated under reduced pressure to give a residue. The residue was purified by column chromatography (SiO₂, Petroleum ether/Ethyl acetate=50/1 to 10/1) to give the title compound (19 g, 91% yield) as a white solid. ¹H NMR (400 MHz, DMSO-d6) δ=6.89 (s, 1H), 6.54 (br d, J=8.3 Hz, 1H), 6.37-6.22 (m, 1H), 1.72-1.62 (m, 1H), 0.55 (s, 4H), 0.41 (br s, 1H), 0.31 (br d, J=4.5 Hz, 2H); LC-MS (ESI⁺) m/z 202.1(M-56+H)⁺.

Step 3-1-(4-Ethynylphenyl)cyclopropanamine. To a solution of tert-butyl N-[1-(4-ethynylphenyl)cyclopropyl]carbamate (2 g, 8 mmol) in DCM (20 mL) was added ZnCl₂ (2.12 g, 15.5 mmol, 728 uL). The mixture was stirred at 25° C. for 12 hrs. On completion, the reaction mixture was partitioned between DCM (20 mL) and H₂O (10 mL). The organic phase was separated, washed with NaCl (5 mL×3), dried over Na₂SO₄, filtered and concentrated under reduced pressure to give a residue. The residue was purified by column chromatography (SiO₂, Petroleum ether/Ethyl acetate=1/5 to 1/2) to give the title compound (1.1 g, 73% yield) as a white solid. LC-MS (ESI+) m/z158.2 (M+H)⁺.

### Phenyl ((S)-1-((2S,4R)-2-((1-(4-ethynylphenyl)cyclopropyl)carbamoyl)-4-hydroxypyrrolidin-1-yl)-3,3-dimethyl-1-oxobutan-2-yl)carbamate (Intermediate MP)

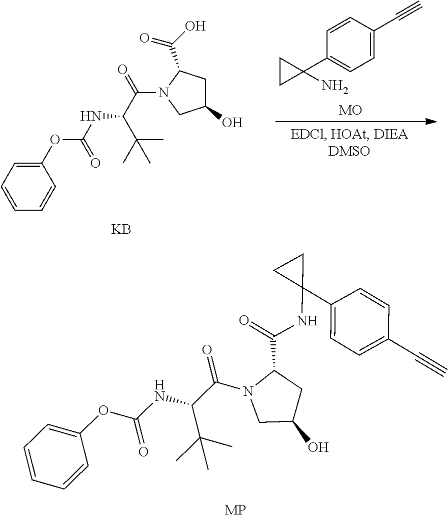 [SMILES: CC(C)(C)[C@H](NC(=O)Oc1ccccc1)C(=O)N1C[C@H](O)C[C@H]1C(=O)O ; C#Cc1ccc(C2(N)CC2)cc1 ; "KB" ; C#Cc1ccc(C2(NC(=O)[C@@H]3C[C@@H](O)CN3C(=O)[C@@H](NC(=O)Oc3ccccc3)C(C)(C)C)CC2)cc1]

To a solution of (2S,4R)-1-[(2S)-3,3-dimethyl-2-(phenoxycarbonylamino)butanoyl]-4-hydroxy-pyrrolidine-2-carboxylic acid (579 mg, 1.59 mmol, Intermediate KB) in DMSO (11 mL) was added DIEA (616 mg, 4.77 mmol, 831 uL), EDCI (365 mg, 1.91 mmol), and HOAt (260 mg, 1.91 mmol, 267 uL) DIEA (617 mg, 4.77 mmol, 831 uL). After 10 min 1-(4-ethynylphenyl)cyclopropanamine (250 mg, 1.59 mmol, Intermediate MO) was added and the mixture was stirred at 25° C. for 12 hr. The crude product was purified by reversed-phase HPLC(0.1% FA condition) to give the title compound (322 mg, 36% yield, FA) as a yellow solid. LC-MS (ESI⁺) m/z504.1 (M+H)⁺.

### Tert-butyl 4-(pyrrolidin-3-ylmethyl)piperazine-1-carboxylate (Intermediate MQ)

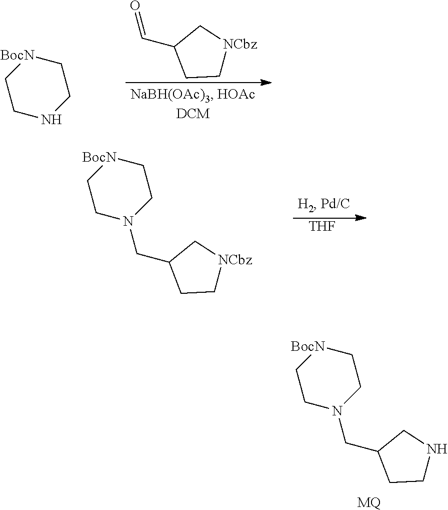

Step 1-Tert-butyl 4-((1-((benzyloxy)carbonyl)pyrrolidin-3-yl)methyl)piperazine-1-carboxylate. To a solution of tert-butyl piperazine-1-carboxylate (6.21 g, 27.9 mmol, CAS #143238-38-4) and benzyl 3-formylpyrrolidine-1-carboxylate (5 g, 21.4 mmol, CAS #276872-86-7) was added HOAc (3.86 g, 64.3 mmol) in DCM (40 mL) was added HOAc (3.86 g, 64.3 mmol) and the reaction mixture was stirred at 25° C. for 30 minutes. Then NaBH(OAc)₃ (13.6 g, 64.3 mmol) was added and stirred at 0-25° C. for 12 hrs. The reaction mixture was partitioned between DCM (100 mL) and water (100 mL). The organic phase was separated, dried over Na₂SO₄, filtered and concentrated under reduced pressure to give a residue. The residue was purified by column chromatography (SiO₂, DCM: MeOH=50:1 to 10:1) to give the title compound (3.7 g, 41% yield) as a yellow oil. ¹H NMR (400 MHz, DMSO-d₆) δ=7.37-7.30 (m, 4H), 5.05 (s, 2H), 3.46-3.34 (m, 2H), 3.32-3.21 (m, 6H), 2.99 (ddd, J=7.2, 10.8, 13.2 Hz, 1H), 2.48-2.19 (m, 7H), 1.96-1.86 (m, 1H), 1.59-1.44 (m, 1H), 1.39 (s, 9H). LC-MS (ESI+) m/z 404.5 (M+H)⁺.

Step 2-Tert-butyl 4-(pyrrolidin-3-ylmethyl)piperazine-1-carboxylate. To a solution of tert-butyl 4-[(1-benzyloxycarbonylpyrrolidin-3-yl)methyl]piperazine-1-carboxylate (2.5 g, 6.20 mmol) in THF (30 mL) was added Pd/C (10 wt %, 3 g) under N₂ atmosphere. The suspension was degassed and purged with H₂ three times. The mixture was then stirred under H₂ (15 Psi) at 25° C. for 12 hrs. The reaction mixture was filtered and concentrated under reduced pressure to give the title compound (1.5 g) as a black oil. LC-MS (ESI+) m/z 270.2 (M+H)⁺.

### 2-((5R)-5-methyl-6-(5-(3-(piperazin-1-ylmethyl)pyrrolidin-1-yl)pyrimidin-2-yl)-6,7,8,9-tetrahydro-5H-pyrido[3′,4′:4,5]pyrrolo[2,3-c]pyridazin-3-yl)phenol (Intermediate MR)

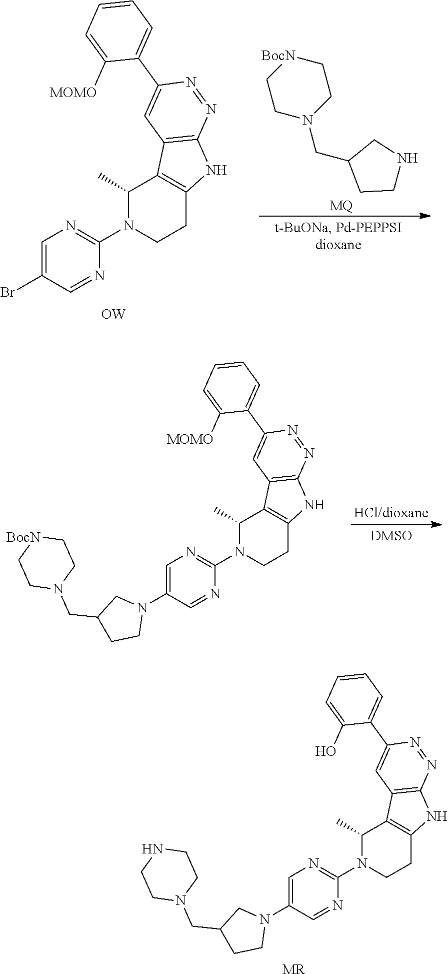

Step 1-Tert-butyl 4-((1-(2-((R)-3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)pyrrolidin-3-yl)methyl)piperazine-1-carboxylate. A mixture of(3R)-4-(5-bromopyrimidin-2-yl)-12-[2-(methoxymethoxy)phenyl]-3-methyl-4,8,10,11-tetrazatricyclo[7.4.0.0^{2,7}]trideca-1(9),2(7),10,12-tetraene (250 mg, 520 umol, Intermediate OW), tert-butyl 4-(pyrrolidin-3-ylmethyl)piperazine-1-carboxylate (210 mg, 779 umol, Intermediate MQ), t-BuONa (2 M, 779 uL), 1,3-bis[2,6-bis(1-propylbutyl)phenyl]-4,5-dichloro-2H-imidazol-1-ium-2-ide;3-chloropyridine dichloropalladium (50.5 mg, 51.9 umol) in dioxane (4 mL) was degassed and purged with N₂ three times. Then the mixture was stirred at 105° C. for 12 hours under N₂ atmosphere. On completion, the reaction mixture was partitioned between ethyl acetate (60 mL) and water (40 mL). The organic phase was separated, washed with brine 40 mL (20 mL×2), dried over Na₂SO₄, filtered and concentrated under reduced pressure to give a residue. The residue was purified by column chromatography (SiO₂, DCM/MeOH=100/1 to 10/1) to give the title compound (1.8 g, 52% yield) as a yellow solid. LC-MS (ESI+) m/z 670.5 (M+H)⁺.

Step 2-2-((5R)-5-methyl-6-(5-(3-(piperazin-1-ylmethyl)pyrrolidin-1-yl)pyrimidin-2-yl)-6,7,8,9-tetrahydro-5H-pyrido[3′,4′:4,5]pyrrolo[2,3-c]pyridazin-3-yl)phenol. To a solution of tert-butyl 4-[[1-[2-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-5-yl]pyrrolidin-3-yl]methyl]piperazine-1-carboxylate (1 g, 2 mmol) was added in DCM (10 mL) and HCl/dioxane (2 mL). The mixture was stirred at 25° C. for 2 hrs. On completion, the reaction mixture was concentrated under reduced pressure to give a residue. The crude product was purified by reversed-phase HPLC (0.1% FA condition) to give the title compound (400 mg, 42% yield, FA) as a yellow solid. LC-MS (ESI+) m/z 526.3 (M+H)⁺.

### (R)-2-(6-(2-([1,4′-bipiperidin]-4-yl)-2-azaspiro[3.3]heptan-6-yl)-5-methyl-6,7,8,9-tetrahydro-5H-pyrido[3′,4′:4,5]pyrrolo[2,3-c]pyridazin-3-yl)phenol (Intermediate MS)

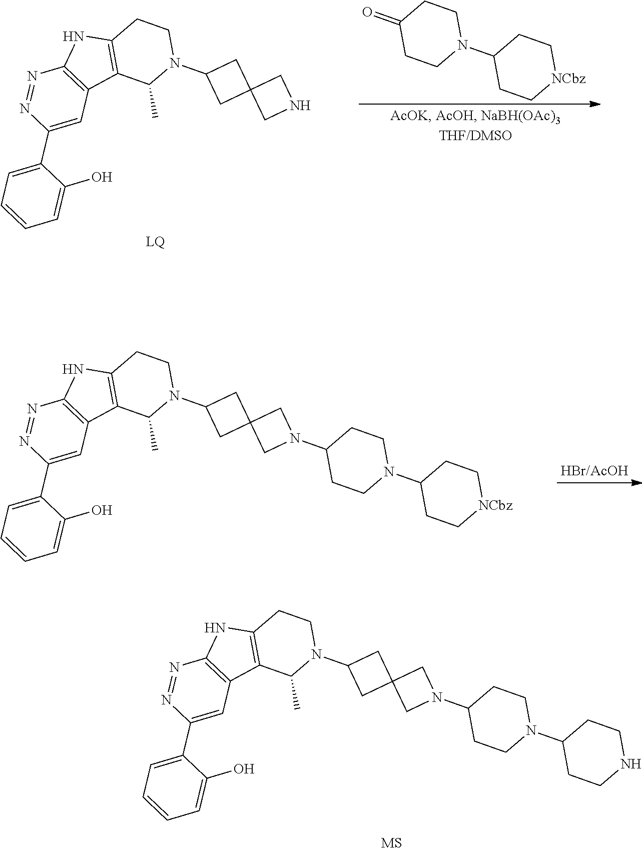 [SMILES: C[C@@H]1c2c([nH]c3nnc(-c4ccccc4O)cc23)CCN1C1CC2(CNC2)C1 ; O=C1CCN(C2CCN(C(=O)OCc3ccccc3)CC2)CC1 ; C[C@@H]1c2c([nH]c3nnc(-c4ccccc4O)cc23)CCN1C1CC2(C1)CN(C1CCN(C3CCN(C(=O)OCc4ccccc4)CC3)CC1)C2 ; C[C@@H]1c2c([nH]c3nnc(-c4ccccc4O)cc23)CCN1C1CC2(C1)CN(C1CCN(C3CCNCC3)CC1)C2]

Step 1-(R)-benzyl 4-(6-(3-(2-hydroxyphenyl)-5-methyl-7,8-dihydro-5H-pyrido[3′,4′:4,5]pyrrolo[2,3-c]pyridazin-6(9H)-yl)-2-azaspiro[3.3]heptan-2-yl)-[1,4′-bipiperidine]-1′-carboxylate. To a solution of 2-[(3R)-4-(2-azaspiro[3.3]heptan-6-yl)-3-methyl-4,8,10,11-tetrazatricyclo[7.4.0.02,7]trideca-1(9),2(7),10,12-tetraen-12-yl]phenol (600 mg, 1.60 mmol, Intermediate LQ) in THF (15 mL) and DMSO (3 mL) was added AcOK (470 mg, 4.79 mmol) at 25° C. and the mixture was stirred for 0.5 hours. Next, AcOH (288 mg, 4.79 mmol, 274 uL), and benzyl 4-(4-oxo-1-piperidyl)piperidine-1-carboxylate (1.01 g, 3.20 mmol, CAS #880462-12-4) was added at 25° C. and the mixture was stirred for 1.5 hours. At last NaBH(OAc)₃ (1.02 g, 4.79 mmol) was added at 0° C. and the mixture was stirred at 25° C. for 12 hours. On completion, water (2 ml) was added to the mixture and then concentrated under reduced pressure to give a residue. The residue was purified by prep-HPLC (FA) to give the title compound (746 mg, 64% yield) as a yellow solid. LC-MS (ESI⁺) m/z 676.6 (M+H)⁺.

Step 2-(R)-2-(6-(2-([1,4′-bipiperidin]-4-yl)-2-azaspiro[3.3]heptan-6-yl)-5-methyl-6,7,8,9-tetrahydro-5H-pyrido[3′,4′:4,5]pyrrolo[2,3-c]pyridazin-3-yl)phenol. To a solution of benzyl 4-[4-[6-[(3R)-12-(2-hydroxyphenyl)-3-methyl-4,8,10,11-tetrazatricyclo[7.4.0.02,7]trideca-1(9),2(7),10,12-tetraen-4-yl]-2-azaspiro[3.3]heptan-2-yl]-1-piperidyl]piperidine-1-carboxylate (254 mg, 376 umol) was added hydrogen bromide (2.98 g, 36.8 mmol, 2 mL). The mixture was stirred at 25° C. for 2 hours. On completion, the reaction was concentrated under reduced pressure to give a residue. The residue was purified by prep-HPLC (FA) to give the title compound (157 mg, 71% yield) as a yellow solid. LC-MS (ESI⁺) m/z 542.2 (M+H)⁺.

### 3-(2-(aminomethyl)-5-ethynylphenoxy)-N,N-dimethylpropan-1-amine (Intermediate MT)

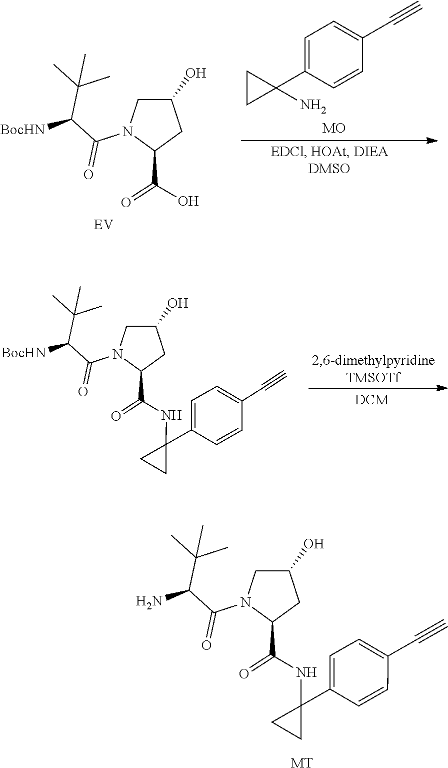

Step 1-Tert-butyl 2-(3-(dimethylamino)propoxy)-4-ethynylbenzylcarbamate. To a solution of (2S,4R)-1-[(2S)-2-(tert-butoxycarbonylamino)-3,3-dimethyl-butanoyl]-4-hydroxy-pyrrolidine-2-carboxylic acid (1.10 g, 3.18 mmol, Intermediate EV) in DMSO (25 mL) was added EDCI (732 mg, 3.82 mmol), DIEA (1.23 g, 9.54 mmol, 1.66 mL) and HOAt (519 mg, 3.82 mmol, 534 uL). After 10 mins was added 1-(4-ethynylphenyl)cyclopropanamine (500 mg, 3.18 mmol, Intermediate MO) and the mixture was stirred at 25° C. for 12 hrs. On completion, the crude product was purified by reversed-phase HPLC(0.1% FA condition) to give the title compound (475 mg, 27% yield, FA) as a white solid. LC-MS (ESI⁺) m/z 484.2 (M+H)⁺.

Step 2-3-(2-(aminomethyl)-5-ethynylphenoxy)-N,N-dimethylpropan-1-amine. To a solution of tert-butyl N-[(1S)-1-[(2S,4R)-2-[[1-(4-ethynylphenyl)cyclopropyl]carbamoyl]-4-hydroxy-pyrrolidine-1-carbonyl]-2,2-dimethyl-propyl]carbamate (150 mg, 310 umol) in DCM (2 mL) was added TMSOTf (207 mg, 931 umol, 168 uL) and 2,6-dimethylpyridine (166 mg, 1.55 mmol, 181 uL). The mixture was stirred at 25° C. for 1 hr. On completion, the crude product was purified by reversed-phase HPLC(0.1% FA condition) to give the title compound (95 mg, 68% yield, FA) as a yellow solid. LC-MS (ESI⁺) m/z 384.1(M+H)⁺.

### Phenyl ((S)-1-((2S,4R)-2-((4-ethynyl-2-methoxybenzyl)carbamoyl)-4-hydroxypyrrolidin-1-yl)-3,3-dimethyl-1-oxobutan-2-yl)carbamate (Intermediate MU)

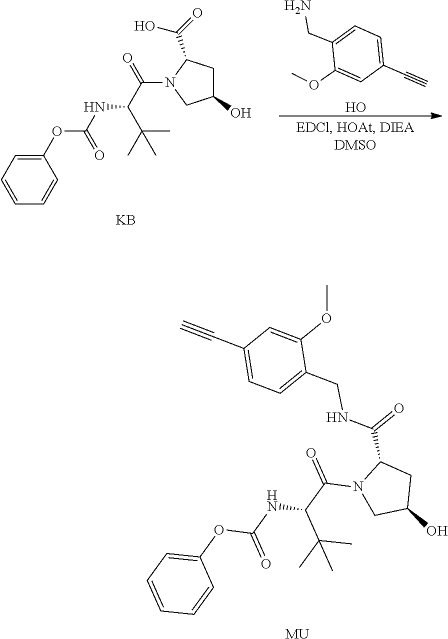

A mixture of (4-ethynyl-2-methoxy-phenyl)methanamine (3 g, 18.6 mmol, Intermediate HO), (2S,4R)-1-[(2S)-3,3-dimethyl-2-(phenoxycarbonylamino)butanoyl]-4-hydroxy-pyrrolidine-2-carboxylic acid (8.14 g, 22.3 mmol, Intermediate KB), DIEA (28.9 g, 223 mmol), and HATU (8.49 g, 22.3 mmol) in DMF (30 mL) was degassed and purged with N₂ three times. Then the mixture was stirred at 25° C. for 12 hrs under N₂ atmosphere. The reaction mixture was concentrated under reduced pressure to remove EA. The residue was diluted with water (50 mL) and extracted with EA (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=1/0 to 1/3) to give the title compound (3.8 g, 32% yield) as a yellow solid. LC-MS (ESI⁺) m/z 388.3 (M+H)⁺.

### 2-((5R)-5-methyl-6-(pyrrolidin-3-yl)-6,7,8,9-tetrahydro-5H-pyrido[3′,4′:4,5]pyrrolo[2,3-c]pyridazin-3-yl)phenol (Intermediate MV)

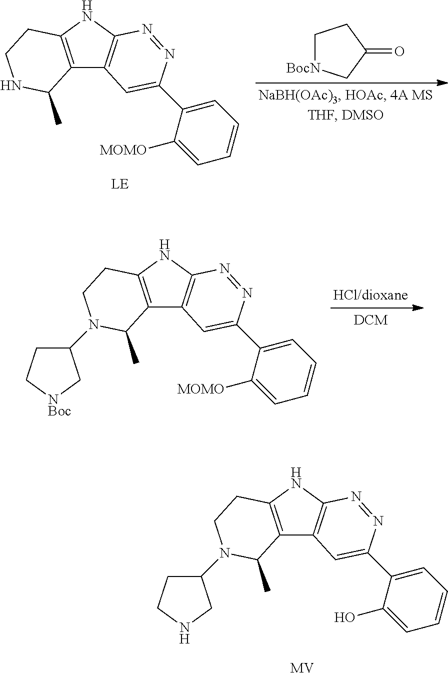

Step 1-Tert-butyl 3-((R)-3-(2-(methoxymethoxy)phenyl)-5-methyl-7,8-dihydro-5H-pyrido[3′,4′:4,5]pyrrolo[2,3-c]pyridazin-6(9H)-yl)pyrrolidine-1-carboxylate. To a solution of (3R)-12-[2-(methoxymethoxy)phenyl]-3-methyl-4,8,10,11-tetrazatricyclo[7.4.0.0^{2,7}]trideca-1(9),2(7),10,12-tetraene (1 g, 3 mmol, Intermediate LE) in THF (40 mL) and DMSO (10 mL) was added HOAc (92.6 mg, 1.54 mmol), 4 Å molecular sieves (3 g, 3 mmol) tert-butyl 3-oxopyrrolidine-1-carboxylate (1.14 g, 6.17 mmol) at 60° C. After 1 hr was added NaBH(OAc)₃ (1.96 g, 9.25 mmol) at 0° C. and the mixture was stirred at 25° C. for 12 hrs. On completion, the reaction mixture was partitioned between EA (50 mL) and H₂O (30 mL). The organic phase was separated, washed with NaCl 30 mL (10 mL×3), dried over Na₂SO₄, filtered and concentrated under reduced pressure to give a residue. The residue was purified by column chromatography (SiO₂, Petroleum ether/Ethyl acetate=10/1 to 1/1) to give the title compound (1.02 g, 62% yield) as a yellow solid. LC-MS (ESI⁺) m/z=494.4 (M+H)⁺.

Step 2-2-((5R)-5-methyl-6-(pyrrolidin-3-yl)-6,7,8,9-tetrahydro-5H-pyrido[3′,4′:4,5]pyrrolo[2,3-c]pyridazin-3-yl)phenol. To a solution of tert-butyl 3-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]pyrrolidine-1-carboxylate (920 mg, 1.86 mmol) in DCM (12 mL) was added HCl/dioxane (1 M, 1.86 mL). The mixture was then stirred at 25° C. for 1 hr. On completion, the reaction mixture was concentrated under reduced pressure to give the title compound (650 mg, HCl) was obtained as a white solid. LC-MS (ESI⁺) m/z=350.1(M+H)⁺.

### 2-((5R)-6-(1-(1-(2-azaspiro[3.3]heptan-6-yl)piperidin-4-yl)pyrrolidin-3-yl)-5-methyl-6,7,8,9-tetrahydro-5H-pyrido[3′,4′:4,5]pyrrolo[2,3-c]pyridazin-3-yl)phenol (Intermediate MW)

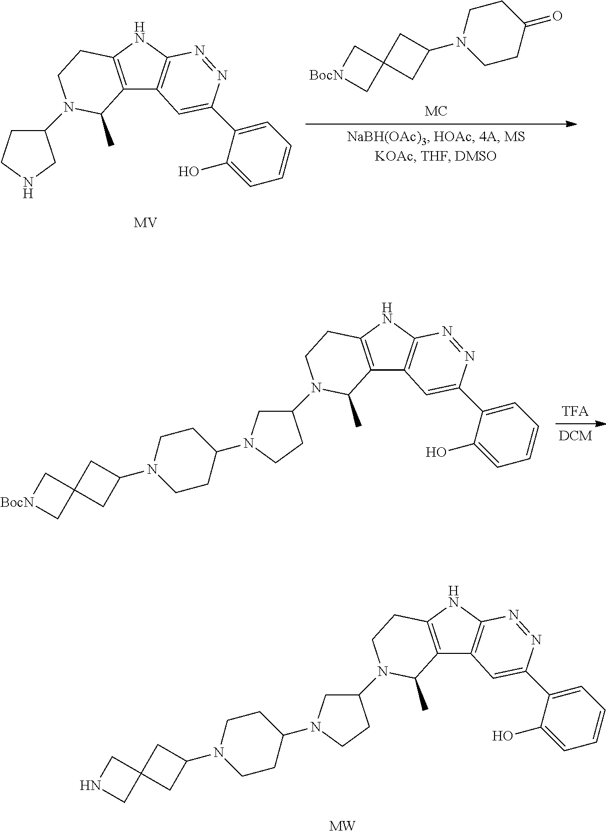

Step 1-Tert-butyl 6-(4-(3-((R)-3-(2-hydroxyphenyl)-5-methyl-7,8-dihydro-5H-pyrido[3′,4′:4,5]pyrrolo[2,3-c]pyridazin-6(9H)-yl)pyrrolidin-1-yl)piperidin-1-yl)-2-azaspiro[3.3]heptane-2-carboxylate. To a solution of 2-[(3R)-3-methyl-4-pyrrolidin-3-yl-4,8,10,11-tetrazatricyclo[7.4.0.0^{2,7}]trideca-1(9),2(7),10,12-tetraen-12-yl]phenol (650 mg, 1.68 mmol, HCl, Intermediate MV) in THF (16 mL) and DMSO (4 mL) was added KOAc (496 mg, 5.05 mmol). After 1 hr, 4 Å molecular sieves (1 g, 1.68 mmol), HOAc (404 mg, 6.74 mmol), and tert-butyl 6-(4-oxo-1-piperidyl)-2-azaspiro[3.3]heptane-2-carboxylate (496 mg, 1.68 mmol, Intermediate MC) were added to the mixture at 40° C. After 1 hr, NaBH(OAc)₃ (1.07 g, 5.05 mmol) was added at 0° C., then the mixture was stirred at 25° C. for 10 hr. On completion, the mixture was filtered and concentrated under reduced pressure to give a residue. The crude product was purified by reversed-phase HPLC(0.1% NH₃•H₂O) to give the title compound (600 mg, 53% yield) as a white solid. LC-MS (ESI+) m/z=628.5 (M+H)+

Step 2-2-((5R)-6-(1-(1-(2-azaspiro[3.3]heptan-6-yl)piperidin-4-yl)pyrrolidin-3-yl)-5-methyl-6,7,8,9-tetrahydro-5H-pyrido[3′,4′:4,5]pyrrolo[2,3-c]pyridazin-3-yl)phenol. To a solution of tert-butyl 6-[4-[3-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrrolidin-1-yl]-1-piperidyl]-2-azaspiro[3.3]heptane-2-carboxylate (500 mg, 800 umol) in DCM (5 mL) was added TFA (770 mg, 6.75 mmol, 0.5 mL).The mixture was then stirred at 25° C. for 0.5 hr. On completion, the reaction mixture was filtered and concentrated under reduced pressure to give the title compound (410 mg, TFA) as a yellow solid. LC-MS (ESI⁺) m/z=528.3 (M+H)⁺.

### (2S,4R)-1-((S)-2-amino-3,3-dimethylbutanoyl)-N—((S)-1-(4-ethynylphenyl)ethyl)-4-hydroxypyrrolidine-2-carboxamide (Intermediate MX)

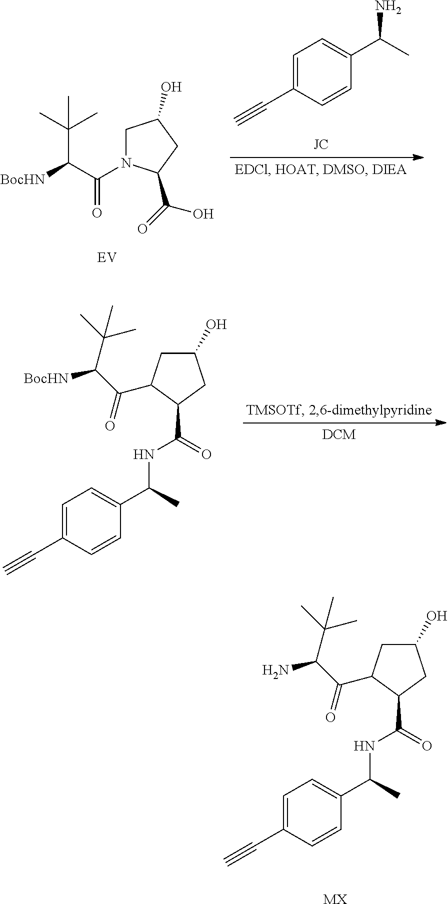

Step 1-Tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate. To a mixture of (S)-1-(4-ethynylphenyl)ethanamine (1.5 g, 7.84 mmol, Intermediate JC), (2S,4R)-1-((S)-2-((tert-butoxycarbonyl)amino)-3,3-dimethylbutanoyl)-4-hydroxypyrrolidine-2-carboxylic acid (2.79 g, 8.63 mmol, Intermediate EV) in DMSO (15 mL) was added EDCI (2.26 g, 11.7 mmol), HOAT (1.6 g, 11.7 mmol) and DIEA (3.04 g, 23.5 mmol). The mixture was stirred at 25° C. for 1 hr. On completion, the reaction mixture was filtered and concentrated under reduced pressure to give a residue. The crude was purified by prep-HPLC (0.1% NH₃·H₂O) to give the title compound (3.39 g) as a white solid. LC-MS (ESI+) m/z 472.2 (M+H)⁺.

Step 2-(2S,4R)-1-((S)-2-amino-3,3-dimethylbutanoyl)-N—((S)-1-(4-ethynylphenyl)ethyl)-4-hydroxypyrrolidine-2-carboxamide. To a mixture of tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate (3.39 g, 7.19 mmol) in DCM (15 mL) and was added TMSOTf (4.79 g, 21.6 mmol) and 2,6-dimethyl(115N)pyridine (3.89 g, 35.9 mmol). The mixture was stirred at 25° C. for 30 mins. On completion, the reaction mixture filtered and concentrated under reduced pressure to give a residue. The crude product was purified by prep-HPLC (0.1% FA). After freeze drying, the salt was then purified by prep-HPLC (0.1% NH₃·H₂O) to give the title compound (650 mg, 23% yield) as a yellow solid. LC-MS (ESI+) m/z 372.3 (M+H)⁺.

### Benzyl 6-(4-(4-oxocyclohexyl)piperidin-1-yl)-2-azaspiro[3.3]heptane-2-carboxylate (Intermediate MY)

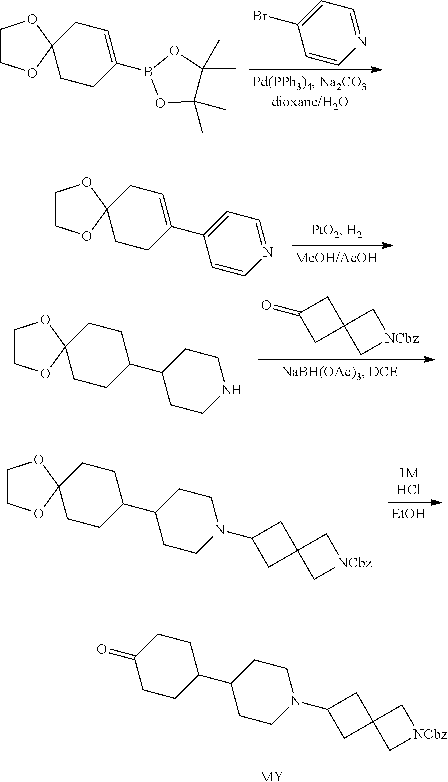

Step 1-4-(1,4-Dioxaspiro[4.5]dec-7-en-8-yl)pyridine. To a solution of 2-(1,4-dioxaspiro[4.5]dec-7-en-8-yl)-4,4,5,5-tetramethyl-1,3,2-dioxaborolane (10 g, 37.6 mmol, CAS #680596-79-6) in 1,4-dioxane (170 mL) and H₂O (19 mL) was added 4-bromopyridine (5.94 g, 37.6 mmol, CAS #1120-87-2), K₂CO₃ (15.58 g, 113 mmol) and cyclopentyl(diphenyl)phosphane dichloromethane dichloropalladium;iron (3.07 g, 3.76 mmol). The mixture was stirred at 85° C. for 12 hrs. On completion, the reaction mixture was quenched with H₂O (100 mL), and then diluted with H₂O (100 mL) and extracted with EA (200 mL×3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=5/1 to 2/1) to give title compound (6.67 g, 79% yield) as a yellow solid. LC-MS (ESI⁺) m/z 218.1 (M+H)⁺.

Step 2-4-(1,4-Dioxaspiro[4.5]decan-8-yl)piperidine. To a solution of 4-(1,4-dioxaspiro[4.5]dec-7-en-8-yl)pyridine (6.67 g, 30.7 mmol) in MeOH (140 mL) and AcOH (70 mL) was added PtO₂ (1.39 g, 6.14 mmol) under nitrogen. Then the mixture was stirred at 25° C. for 12 hrs under H₂ (50 Psi) in a 1000 mL of autoclave. On completion, the reaction mixture was filtered with kieselguhr and the filter cake was washed with MeOH (50 mL). The filtrate was neutralized with Na₂CO₃, filtered and concentrated under reduced pressure to give the title compound (5.2 g). LC-MS (ESI⁺) m/z 226.1 (M+H)⁺.

Step 3-Benzyl 6-(4-(1,4-dioxaspiro[4.5]decan-8-yl)piperidin-1-yl)-2-azaspiro[3.3]heptane-2-carboxylate. To a solution of 4-(1,4-dioxaspiro[4.5]decan-8-yl)piperidine (1 g, 4.44 mmol) in 1,2-dichloroethane (10 mL) was added dropwise benzyl 6-oxo-2-azaspiro[3.3]heptane-2-carboxylate (1.09 g, 4.44 mmol), 4 Å molecular sieves (1 g, 3.72 mmol) and AcOH (223 mg, 3.72 mmol). After addition, the mixture was stirred at 25° C. for 2 hrs, and then sodium triacetoxyboranuide (1.88 g, 8.88 mmol) was added dropwise at 0° C. The resulting mixture was stirred at 25° C. for 4 hrs. On completion, the reaction mixture was quenched with sat. NaHCO₃ (20 mL), and then diluted with H₂O (10 mL), extracted with DCM (30 mL×3) and filtered. The combined organic layers were washed with brine (20 mL), dried over Na₂SO₄, filtered and concentrated under reduced pressure to give a residue. The crude product was purified by reversed-phase HPLC (0.1% FA condition) to give title compound (450 mg, 24% yield, FA salt) as a yellow solid. LC-MS (ESI⁺) m/z 455.5 (M+H)⁺.

Step 4-Benzyl 6-(4-(4-oxocyclohexyl)piperidin-1-yl)-2-azaspiro[3.3]heptane-2-carboxylate. To a solution of benzyl 6-[4-(1,4-dioxaspiro[4.5]decan-8-yl)-1-piperidyl]-2-azaspiro[3.3]heptane-2-carboxylate (450 mg, 900 umol, FA salt) in EtOH (5 mL) was added HCl (1 M, 4.54 mL). The mixture was stirred at 70° C. for 6 hrs. On completion, the reaction mixture was concentrated under reduced pressure to give a residue. The residue was purified by column chromatography (SiO₂, DCM: MeOH=10:1) to give title compound (300 mg, 62% yield) as a yellow oil. LC-MS (ESI⁺) m/z411.4 (M+H)⁺.

### (R)-2-(6-(4-(1-(2-azaspiro[3.3]heptan-6-yl)piperidin-4-yl)cyclohexyl)-5-methyl-6,7,8,9-tetrahydro-5H-pyrido[3′,4′:4,5]pyrrolo[2,3-c]pyridazin-3-yl)phenol (Intermediate MZ)

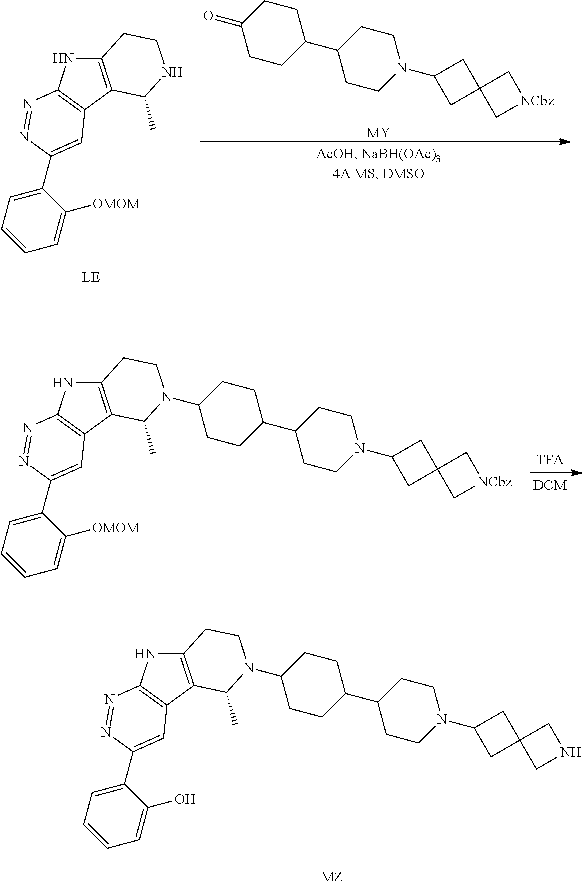

Step 1-(R)-benzyl 6-(4-(4-(3-(2-(methoxymethoxy)phenyl)-5-methyl-7,8-dihydro-5H-pyrido[3′,4′:4,5]pyrrolo[2,3-c]pyridazin-6(9H)-yl)cyclohexyl)piperidin-1-yl)-2-azaspiro[3.3]heptane-2-carboxylate. To a solution of (3R)-12-[2-(methoxymethoxy)phenyl]-3-methyl-4,8,10,11-tetrazatricyclo[7.4.0.02,7]trideca-1(9),2(7),10,12-tetraene (90.1 mg, 278 umol, FA salt, Intermediate LE) in DMSO (4 mL) was added benzyl 6-[4-(4-oxocyclohexyl)-1-piperidyl]-2-azaspiro[3.3]heptane-2-carboxylate (300 mg, 555 umol, Intermediate MY), AcOH (16.7 mg, 278 umol) and 4 Å molecular sieves (420 mg). The mixture was stirred at 60° C. for 2 hrs, and then sodium triacetoxyboranuide (177 mg, 833 umol) was added dropwise at 0° C. The resulting mixture was stirred at 25° C. for 12 hrs. The reaction mixture was quenched with sat. NaHCO₃ (2 mL), and then diluted with H₂O (2 mL), extracted with EA (10 mL×3) and filtered. The combined organic layers were washed with brine (4 mL), dried over Na₂SO₄, filtered and concentrated under reduced pressure to give a residue. The crude product was purified by reversed-phase HPLC (0.1% FA condition) to give title compound (120 mg, 53% yield) as a yellow solid. LC-MS (ESI⁺) m/z719.7 (M+H)⁺.

Step 2-(R)-2-(6-(4-(1-(2-azaspiro[3.3]heptan-6-yl)piperidin-4-yl)cyclohexyl)-5-methyl-6,7,8,9-tetrahydro-5H-pyrido[3′,4′:4,5]pyrrolo[2,3-c]pyridazin-3-yl)phenol. To a solution of benzyl 6-[4-[4-[(3R)-12-[2-(methoxymethoxy)phenyl]-3-methyl-4,8,10,11-tetrazatricyclo[7.4.0.02,7]trideca-1(9),2(7),10,12-tetraen-4-yl]cyclohexyl]-1-piperidyl]-2-azaspiro[3.3]heptane-2-carboxylate (120 mg, 167 umol) in DCM (1 mL) was added TFA (3.27 mL). The mixture was stirred at 40° C. for 12 hrs. On completion, the reaction mixture was concentrated under reduced pressure to give a residue. The crude product was purified by reversed-phase HPLC (0.1% FA condition) to give title compound (65 mg, 67% yield) as a yellow solid. LC-MS (ESI⁺) m/z541.5 (M+H)⁺.

### Tert-butyl 4-((4-oxocyclohexyl)methyl)piperazine-1-carboxylate (Intermediate NA)

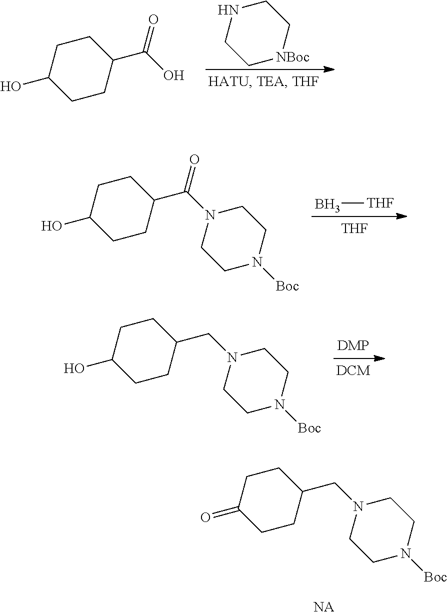

Step 1-Tert-butyl 4-(4-hydroxycyclohexanecarbonyl)piperazine-1-carboxylate. A mixture of 4-hydroxycyclohexanecarboxylic acid (10 g, 69.35 mmol, CAS #17419-81-7) in THF (200 mL) was added HATU (34.28 g, 90.16 mmol) at 0° C., the result solution was stirred for 30 min at 0° C. under N₂ atmosphere. Then tert-butyl piperazine-1-carboxylate (14.21 g, 76.29 mmol, CAS #143238-38-4) and TEA (14.04 g, 138.71 mmol) were added under N₂ atmosphere, and then the mixture was stirred at 25° C. for 16 hr under N₂ atmosphere. On completion, the reaction mixture was quenched with H₂O (200 mL) at 25° C., and then diluted with DCM (200 mL) and extracted with DCM (200 mL×3). The combined organic layers were washed with NaCl (200 mL×3), dried over Na₂SO₄, filtered and concentrated under reduced pressure to give a residue. The crude product was triturated with EA at 25° C. for 30 min. Then the residue was purified by column chromatography (SiO₂, Petroleum ether/Ethyl acetate=10/1 to 0/1) to give the title compound (11 g, 51% yield) as a white solid. ¹H NMR (400 MHz, CHLOROFORM-d) δ ppm 1.48 (s, 9H) 1.51-1.83 (m, 4H) 1.60 (s, 2H) 1.75-1.83 (m, 2H) 2.03-2.12 (m, 2H) 2.37-2.47 (m, 1H) 3.40-3.48 (m, 5H) 3.55-3.70 (m, 3H). LC-MS (ESI⁺) m/z 335.1 (M+Na)⁺.

Step 2-Tert-butyl 4-((4-hydroxycyclohexyl)methyl)piperazine-1-carboxylate. To a solution of tert-butyl 4-(4-hydroxycyclohexanecarbonyl)piperazine-1-carboxylate (2 g, 6.40 mmol) in THF (24 mL) was dropwise added BH₃.THF (1 M, 23.69 mL) at 0° C. After addition, the resulting mixture was stirred at 80° C. for 16 hr. On completion, the reaction mixture was cooled to 0° C., followed by the addition of MeOH (20 mL) at 0° C. slowly. The mixture was stirred at rt for 12 hr and concentrated under reduced pressure. The residue was diluted with H₂O (20 mL) and extracted with DCM (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=3/1 to 1/1) to give the title compound (1 g, 52% yield) as a white solid. ¹H NMR (400 MHz, CHLOROFORM-d) δ ppm 0.88-0.98 (m, 2H) 1.17-1.32 (m, 3H) 1.46 (s, 9H) 1.82-1.89 (m, 2H) 1.96-2.03 (m, 2H) 2.12 (d, J=7.13 Hz, 2H) 2.33-2.31 (m, 4H) 3.38-3.44 (m, 4H) 3.52-3.61 (m, 1H). LC-MS (ESI+) m/z 299.2 (M+H)⁺.

Step 3-tert-butyl 4-((4-oxocyclohexyl)methyl)piperazine-1-carboxylate. To a solution of tert-butyl 4-[(4-hydroxycyclohexyl)methyl]piperazine-1-carboxylate (0.9 g, 3 mmol) in DCM (10 mL) was dropwise added DMP (1.92 g, 4.52 mmol) at 0° C. After addition, the resulting mixture was stirred at 25° C. for 3 hr. On completion, the reaction mixture was quenched by addition of sat. NaHCO₃ (10 mL) at 25° C., and then diluted with DCM (10 mL) and extracted with DCM (10 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=10/1 to 0/1) to give the title compound (0.8 g, 2.70 mmol, 90% yield) as a yellow solid. ¹H NMR (400 MHz, CDCl₃-d) 6 ppm 1.47 (s, 9H) 1.97 (m, 1H) 2.06-2.20 (m, 3H) 2.25-2.44 (m, 11H) 3.40-3.49 (m, 4H). LC-MS (ESI⁺) m/z 297.2 (M+H)⁺.

### Tert-butyl 4-(((1S,4s)-4-(4-((R)-3-(2-hydroxyphenyl)-5-methyl-7,8-dihydro-5H-pyrido[3′,4′:4,5]pyrrolo[2,3-c]pyridazin-6(9H)-yl)piperidin-1-yl)cyclohexyl)methyl)piperazine-1-carboxylate (Intermediate NB) tert-butyl 4-(((1R,4r)-4-(4-((R)-3-(2-hydroxyphenyl)-5-methyl-7,8-dihydro-5H-pyrido[3′,4′:4,5]pyrrolo[2,3-c]pyridazin-6(9H)-yl)piperidin-1-yl)cyclohexyl)methyl)piperazine-1-carboxylate (Intermediate NC)

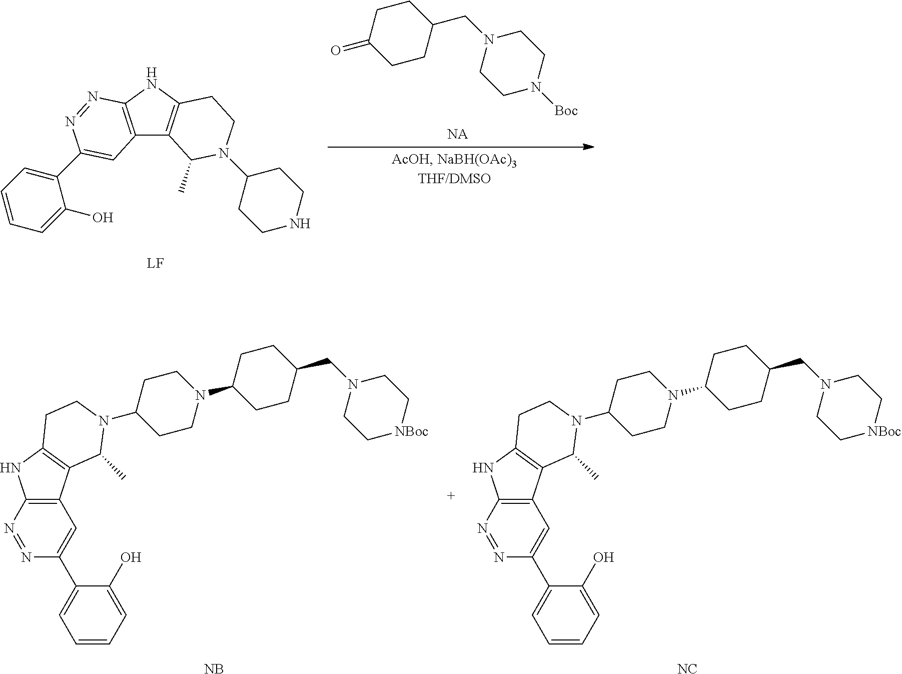 [SMILES: C[C@@H]1c2c([nH]c3nnc(-c4ccccc4O)cc23)CCN1C1CCNCC1 ; CC(C)(C)OC(=O)N1CCN(CC2CCC(=O)CC2)CC1 ; C[C@@H]1c2c([nH]c3nnc(-c4ccccc4O)cc23)CCN1C1CCN([C@H]2CC[C@@H](CN3CCN(C(=O)OC(C)(C)C)CC3)CC2)CC1 ; C[C@@H]1c2c([nH]c3nnc(-c4ccccc4O)cc23)CCN1C1CCN([C@H]2CC[C@H](CN3CCN(C(=O)OC(C)(C)C)CC3)CC2)CC1 ; "NB" ; "NC"]

To a solution of 2-[(3R)-3-methyl-4-(4-piperidyl)-4,8,10,11-tetrazatricyclo[7.4.0.02,7]trideca-1(9),2(7),10,12-tetraen-12-yl]phenol (0.5 g, 1.25 mmol, HCl, Intermediate LF) in THF (8 mL) and DMSO (4 mL) was added TEA (253.03 mg, 2.50 mmol), the pH was adjusted to around 7. Then 4 Å molecular sieves (4 g), AcOH (150.13 mg, 2.50 mmol) and tert-butyl 4-[(4-oxocyclohexyl)methyl]piperazine-1-carboxylate (741.01 mg, 2.50 mmol, Intermediate NA) was added and the mixture was stirred at 40° C. for 2 h. Then NaBH(OAc)₃ (529.96 mg, 2.50 mmol) was added at 0° C. The mixture was stirred at 25° C. for 12 h. On completion, the reaction mixture was filtered and concentrated under reduced pressure to give a residue. The crude product was purified by reversed-phase HPLC (0.1% FA condition). After prep. HPLC purification, the eluent was concentrated to remove organic solvents. The residual aqueous solution was lyophilized to give a yellow foam. The product was then separated by SFC (condition: column: DAICEL CHIRALPAK AD (250 mm*30 mm, 10 um); mobile phase: [0.1% NH₃H₂O IPA]; B %: 70%-70%,5; 40 min) to give Tert-butyl 4-(((1S,4s)-4-(4-((R)-3-(2-hydroxyphenyl)-5-methyl-7,8-dihydro-5H-pyrido[3′,4′:4,5]pyrrolo[2,3-c]pyridazin-6(9H)-yl)piperidin-1-yl)cyclohexyl)methyl)piperazine-1-carboxylate (0.4 g, 47% yield) as a yellow solid(LC-MS (ESI+) m/z 644.6 (M+H)⁺) and tert-butyl 4-(((1R,4r)-4-(4-((R)-3-(2-hydroxyphenyl)-5-methyl-7,8-dihydro-5H-pyrido[3′,4′:4,5]pyrrolo[2,3-c]pyridazin-6(9H)-yl)piperidin-1-yl)cyclohexyl)methyl)piperazine-1-carboxylate (0.3 g, 36% yield) as a yellow solid (LC-MS (ESI+) m/z 644.5 (M+H)⁺)

### 2-((R)-5-methyl-6-(1-((1s,4S)-4-(piperazin-1-ylmethyl)cyclohexyl)piperidin-4-yl)-6,7,8,9-tetrahydro-5H-pyrido[3′,4′:4,5]pyrrolo[2,3-c]pyridazin-3-yl)phenol (Intermediate ND)

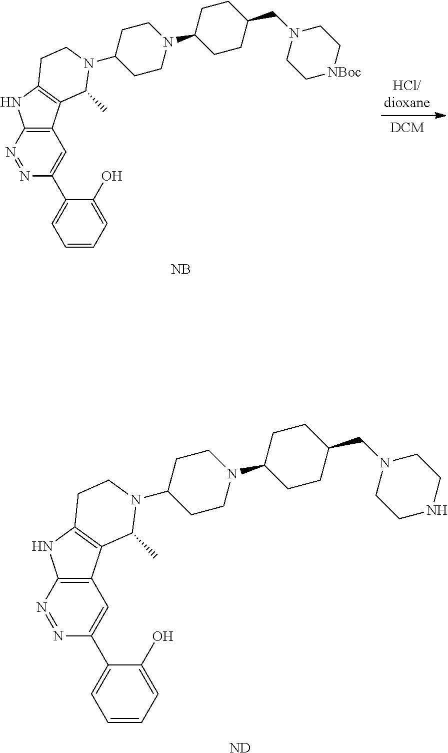 [SMILES: C[C@@H]1c2c([nH]c3nnc(-c4ccccc4O)cc23)CCN1C1CCN([C@H]2CC[C@@H](CN3CCN(C(=O)OC(C)(C)C)CC3)CC2)CC1 ; "NB" ; C[C@@H]1c2c([nH]c3nnc(-c4ccccc4O)cc23)CCN1C1CCN([C@H]2CC[C@@H](CN3CCNCC3)CC2)CC1 ; "ND"]

To a solution of tert-butyl 4-(((1S,4s)-4-(4-((R)-3-(2-hydroxyphenyl)-5-methyl-5,7,8,9-tetrahydro-6H-pyrido[3′,4′:4,5]pyrrolo[2,3-c]pyridazin-6-yl)piperidin-1-yl)cyclohexyl)methyl)piperazine-1-carboxylate (0.4 g, 621.25 umol, Intermediate NB) in DCM (5 mL) was added HCl/dioxane (8 M, 8 mL). The mixture was stirred at 25° C. for 1 hr. On completion, the reaction mixture was concentrated under reduced pressure to give a residue. The crude product was purified by reversed-phase HPLC (0.1% FA condition). After prep. HPLC purification, the eluent was concentrated to remove organic solvents. The residual aqueous solution was lyophilized to give the title compound as a yellow solid (0.25 g, 423.89 umol, 68% yield, FA). LC-MS (ESI+) m/z 544.4 (M+H)⁺.

### 2-((R)-5-methyl-6-(1-((1r,4R)-4-(piperazin-1-ylmethyl)cyclohexyl)piperidin-4-yl)-6,7,8,9-tetrahydro-5H-pyrido[3′,4′:4,5]pyrrolo[2,3-c]pyridazin-3-yl)phenol (Intermediate NE)

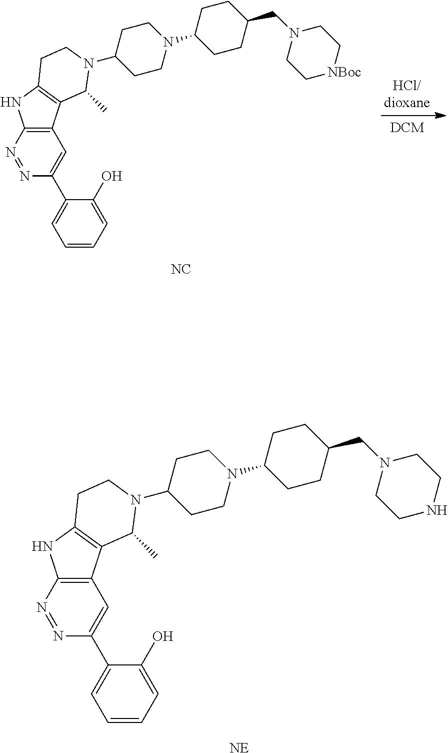 [SMILES: C[C@@H]1c2c([nH]c3nnc(-c4ccccc4O)cc23)CCN1C1CCN([C@H]2CC[C@H](CN3CCN(C(=O)OC(C)(C)C)CC3)CC2)CC1 ; "NC" ; C[C@@H]1c2c([nH]c3nnc(-c4ccccc4O)cc23)CCN1C1CCN([C@H]2CC[C@H](CN3CCNCC3)CC2)CC1 ; "NE"]

To a solution of tert-butyl 4-(((1R,4r)-4-(4-((R)-3-(2-hydroxyphenyl)-5-methyl-5,7,8,9-tetrahydro-6H-pyrido[3′,4′:4,5]pyrrolo[2,3-c]pyridazin-6-yl)piperidin-1-yl)cyclohexyl)methyl)piperazine-1-carboxylate (0.3 g, 500 umol, Intermediate NC) was added HCl/dioxane (8 M, 6 mL) in DCM (5 mL). The mixture was stirred at 25° C. for 1 hr. On completion, the reaction mixture was concentrated under reduced pressure to give a residue. The crude product was purified by reversed-phase HPLC (0.1% FA condition). After prep. HPLC purification, the eluent was concentrated to remove organic solvents. The residual aqueous solution was lyophilized to give the title compound as a white solid (0.15 g, 54% yield, FA). LC-MS (ESI+) m/z 544.5 (M+H)⁺.

### (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate (Intermediate NF) & (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate (Intermediate NG)

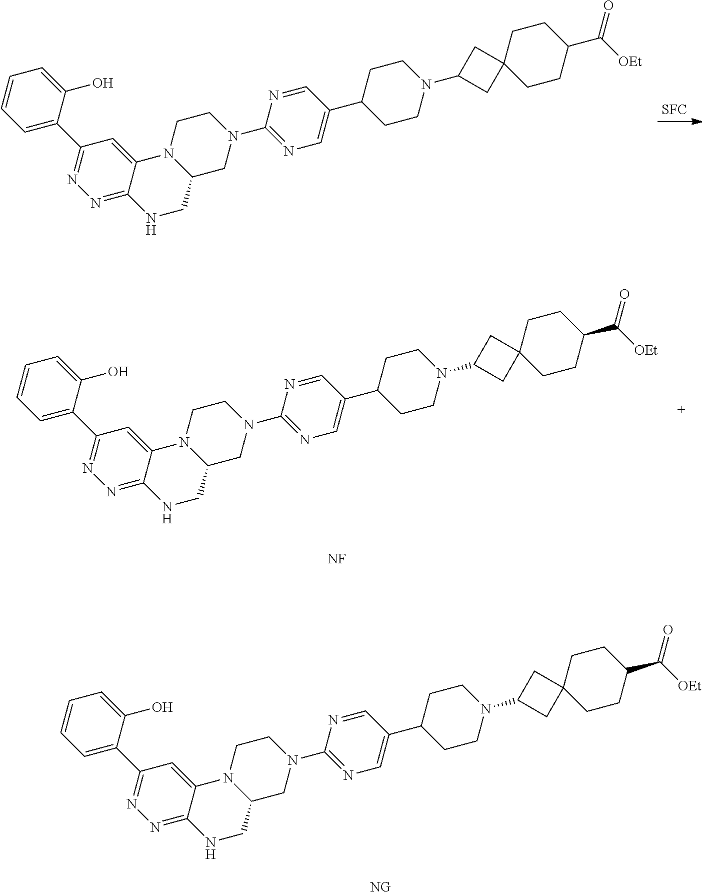

Compound ethyl 2-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]spiro[3.5]nonane-7-carboxylate (7 g, 11 mmol, synthesized via Step 1 of Intermediate M) was purified by SFC. The crude product was purified by SFC (column: DAICEL CHIRALPAK AY-H(250 mm×30 mm, 10 um); mobile phase: [ACN/IPA(0.1% NH₃H2O)]; B %: 60%-60%, A2.6; 350 min) to give (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate (1.8 g) as a white solid (LC-MS (ESI⁺) m z 639.5 (M+H)⁺) and (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylate (1 g) as a white solid (LC-MS (ESI⁺) m/z 639.5 (M+H)⁺).

### (R)-4-(3-(2-hydroxyphenyl)-5-methyl-7,8-dihydro-5H-pyrido[3′,4′:4,5]pyrrolo[2,3-c]pyridazin-6(9H)-yl)cyclohexanone (Intermediate NH)

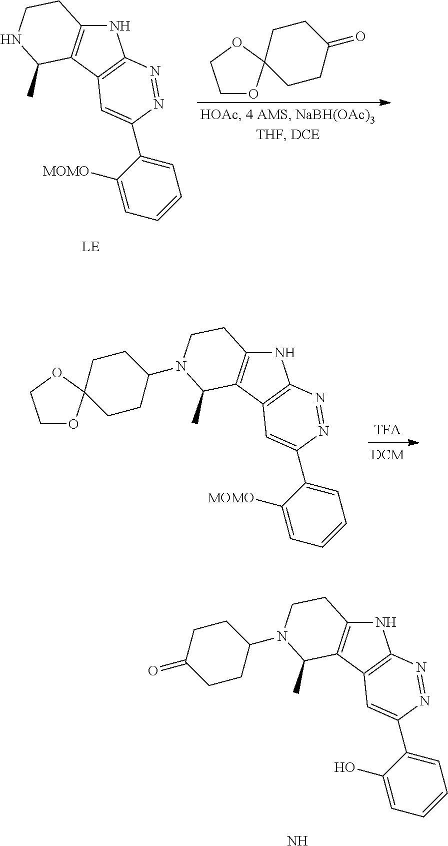

Step 1-(R)-3-(2-(methoxymethoxy)phenyl)-5-methyl-6-(1,4-dioxaspiro[4.5]decan-8-yl)-6,7,8,9-tetrahydro-5H-pyrido[3′,4′:4,5]pyrrolo[2,3-c]pyridazine. To a solution of (3R)-12-[2-(methoxymethoxy)phenyl]-3-methyl-4,8,10,11-tetrazatricyclo[7.4.0.0^{2,7}]trideca-1(9),2(7),10,12-tetraene (2 g, 6.17 mmol, Intermediate LE) and 1,4-dioxaspiro[4.5]decan-8-one (1.93 g, 12.3 mmol, CAS #4746-97-8) was added HOAc (185 mg, 3.08 mmol) in THF (20 mL) and DCE (20 mL) and the mixture was stirred at 80° C. for 2 hrs. Then NaBH(OAc)₃ (2.61 g, 12.3 mmol) was added and the mixture was stirred at 0-25° C. for 12 hrs. The reaction mixture was filtered and concentrated under reduced pressure to give a residue. The crude product was purified by reversed-phase HPLC (0.1% FA condition) to give the title compound (3 g, 90% yield, FA) as a yellow solid. ¹H NMR (400 MHz, DMSO-d₆) δ=12.24-11.76 (m, 1H), 8.17 (s, 1H), 7.95 (s, 1H), 7.70 (dd, J=2.0, 7.6 Hz, 1H), 7.42-7.35 (m, 1H), 7.26-7.21 (m, 1H), 7.14 (dt, J=0.8, 7.6 Hz, 1H), 5.23-5.16 (m, 2H), 4.21 (q, J=6.4 Hz, 1H), 3.94 (s, 1H), 3.84-3.82 (m, 4H), 3.15-3.05 (m, 1H), 2.97 (td, J=4.8, 12.4 Hz, 1H), 2.87-2.75 (m, 2H), 2.74-2.65 (m, 1H), 2.35 (t, J=7.2 Hz, 1H), 1.92 (t, J=7.2 Hz, 1H), 1.84-1.78 (m, 1H), 1.72-1.67 (m, 3H), 1.65-1.63 (m, 1H), 1.54-1.48 (m, 2H), 1.47-1.42 (m, 1H), 1.36 (d, J=6.4 Hz, 3H). LC-MS (ESI+) m/z 465.3 (M+H)⁺.

Step 2-(R)-4-(3-(2-hydroxyphenyl)-5-methyl-7,8-dihydro-5H-pyrido[3′,4′:4,5]pyrrolo[2,3-c]pyridazin-6(9H)-yl)cyclohexanone. To a solution of (3R)-4-(1,4-dioxaspiro[4.5]decan-8-yl)-12-[2-(methoxymethoxy)phenyl]-3-methyl-4,8,10,11-tetrazatricyclo[7.4.0.0^{2,7}]trideca-1(9),2(7),10,12-tetraene (2 g, 4 mmol) in DCM (20 mL) was added TFA (12.3 g, 107 mmol). The mixture was then stirred at 25° C. for 12 hrs. On completion, the reaction mixture was concentrated under reduced pressure to give a residue. The crude product was purified by reversed-phase HPLC (0.1% FA condition) to give the title compound (1.6 g, 70% yield, FA) as a yellow solid. LC-MS (ESI+) m/z 377.1 (M+H)⁺.

### (R)-2-(5-methyl-6-(4-(4-(piperazin-1-ylmethyl)piperidin-1-yl)cyclohexyl)-6,7,8,9-tetrahydro-5H-pyrido[3′,4′:4,5]pyrrolo[2,3-c]pyridazin-3-yl)phenol (Intermediate NI)

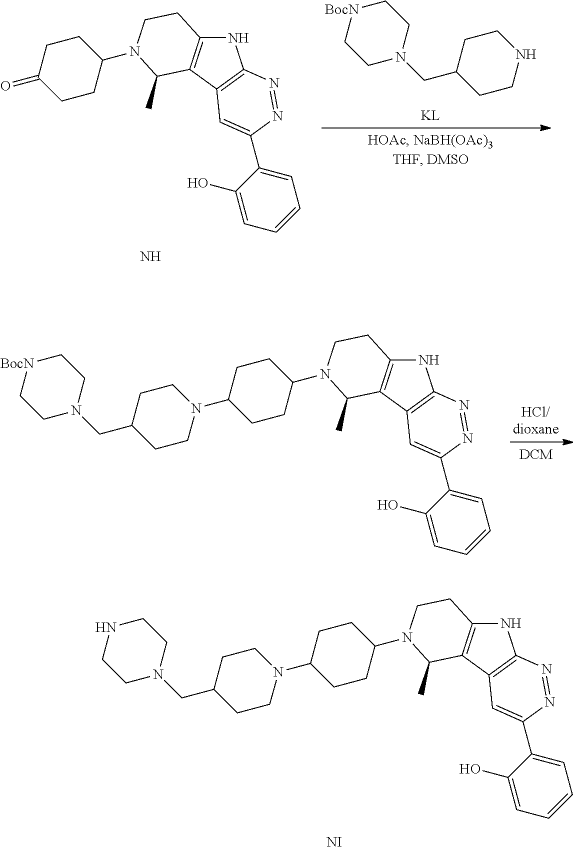

Step 1-(R)-tert-butyl 4-((1-(4-(3-(2-hydroxyphenyl)-5-methyl-7,8-dihydro-5H-pyrido[3′,4′:4,5]pyrrolo[2,3-c]pyridazin-6(9H)-yl)cyclohexyl)piperidin-4-yl)methyl)piperazine-1-carboxylate. To a solution of tert-butyl 4-(4-piperidylmethyl)piperazine-1-carboxylate (565 mg, 1.99 mmol, Intermediate KL) and 4-[(3R)-12-(2-hydroxyphenyl)-3-methyl-4,8,10,11-tetrazatricyclo[7.4.0.0^{2,7}]trideca-1(9),2(7),10,12-tetraen-4-yl]cyclohexanone (500 mg, 1.33 mmol, Intermediate NH) in THF (8 mL) and DMSO (4 mL) was added HOAc (160 mg, 2.66 mmol) and the mixture was stirred at 60° C. for 2 hrs. Then NaBH(OAc)₃ (845 mg, 3.98 mmol) was added and the mixture was stirred at 0-25° C. for 12 hrs. The reaction mixture was filtered and concentrated under reduced pressure to give a residue. The crude product was purified by reversed-phase HPLC (0.1% FA condition) to give the title compound (300 mg, 23% yield, FA) as a yellow solid. LC-MS (ESI+) m/z 644.6 (M+H)⁺.

Step 2-(R)-2-(5-methyl-6-(4-(4-(piperazin-1-ylmethyl)piperidin-1-yl)cyclohexyl)-6,7,8,9-tetrahydro-5H-pyrido[3′,4′:4,5]pyrrolo[2,3-c]pyridazin-3-yl)phenol. To a solution of tert-butyl 4-[[1-[4-[(3R)-12-(2-hydroxyphenyl)-3-methyl-4,8,10,11-tetrazatricyclo[7.4.0.0^{2,7}]trideca-1(9),2(7),10,12-tetraen-4-yl]cyclohexyl]-4-piperidyl]methyl]piperazine-1-carboxylate (300 mg, 466 umol) was added in DCM (3 mL) and HCl/dioxane (1 mL). Then the mixture was stirred at 25° C. for 1 hr. On completion, the reaction mixture was concentrated under reduced pressure to give a residue. The crude product was purified by reversed-phase HPLC (0.1% FA condition) to give the title compound (270 mg, 96% yield, FA) as a yellow solid. LC-MS (ESI+) m/z 544.3 (M+H)⁺.

### (2S,4R)-1-((S)-2-(2-bromoacetamido)-3,3-dimethylbutanoyl)-N-(4-ethynylbenzyl)-4-hydroxypyrrolidine-2-carboxamide (Intermediate NJ)

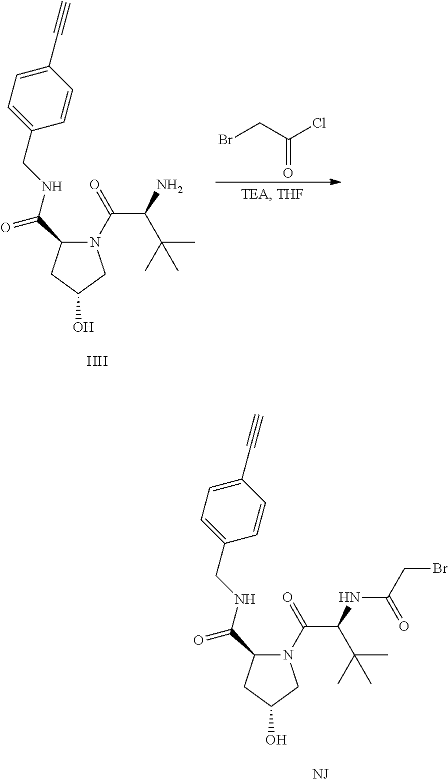

To a solution of (2S,4R)-1-[(2S)-2-amino-3,3-dimethyl-butanoyl]-N-[(4-ethynylphenyl)methyl]-4-hydroxy-pyrrolidine-2-carboxamide (500 mg, 1.40 mmol, Intermediate HH) in THE (5 mL) was added TEA (283 mg, 2.80 mmol) and 2-bromoacetyl chloride (286 mg, 1.82 mmol) in THF (5 mL) at 0° C. The mixture was then stirred at 0-25° C. for 3 hrs. On completion, the reaction mixture was quenched with MeOH (0.1 mL) at 25° C., and then filtered and concentrated under reduced pressure to give a residue. The residue was purified by prep-HPLC (FA condition) to give the title compound (410 mg, 53% yield, FA) as a pink solid. LC-MS (ESI⁺) m/z 480.0 (M+H+2)⁺.

### (S)-2-(8-([1,4′-bipiperidin]-4-yl)-6,6a,7,8,9,10-hexahydro-5H-pyrazino[1′,2′:4,5]pyrazino[2,3-c]pyridazin-2-yl)phenol (Intermediate NK)

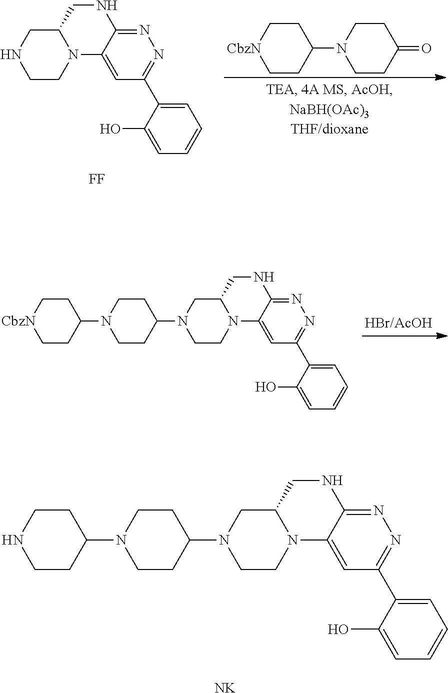 [SMILES: Oc1ccccc1-c1cc2c(nn1)NC[C@H]1CNCCN21 ; O=C1CCN(C2CCN(C(=O)OCc3ccccc3)CC2)CC1 ; "FF" ; O=C(OCc1ccccc1)N1CCC(N2CCC(N3CCN4c5cc(-c6ccccc6O)nnc5NC[C@H]4C3)CC2)CC1 ; Oc1ccccc1-c1cc2c(nn1)NC[C@H]1CN(C3CCN(C4CCNCC4)CC3)CCN21 ; "NK"]

Step 1-(S)-benzyl 4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidine]-1′-carboxylate. To a solution of 2-[(1OR)-1,5,6,8,12-pentazatricyclo[8.4.0.0^{2,7}]tetradeca-2,4,6-trien-4-yl]phenol (1.44 g, 4.05 mmol, 2 HCl, Intermediate FF) in THF (10 mL) and DMSO (5 mL) was added 4 Å molecular sieves (1.5 g) and TEA (1.23 g, 12.1 mmol). The mixture was then stirred at 25° C. for 1 hr. Then benzyl 4-(4-oxo-1-piperidyl)piperidine-1-carboxylate (3.2 g, 10 mmol, CAS #880462-12-4) and AcOH (971 mg, 16.1 mmol) was added and stirred for 2 hrs. Then NaBH(OAc)₃ (2.57 g, 12.1 mmol) was added at 0° C. The mixture was stirred for 2 hours at 25° C. On completion, the reaction mixture was quenched with H₂O (0.5 mL) at 0° C., and then filtered and concentrated under reduced pressure to give a residue. The residue was purified by prep-HPLC (NH₃.H₂O condition) to give the title compound (1.3 g, 45% yield) as a yellow solid. LC-MS (ESI⁺) m/z 584.4 (M+H)⁺.

Step 2-(S)-2-(8-([1,4′-bipiperidin]-4-yl)-6,6a,7,8,9,10-hexahydro-5H-pyrazino[1′,2′:4,5]pyrazino[2,3-c]pyridazin-2-yl)phenol. To a solution of benzyl 4-[4-[(10S)-4-(2-hydroxyphenyl)-1,5,6,8,12-pentazatricyclo[8.4.0.0^{2,7}]tetradeca-2,4,6-trien-12-yl]-1-piperidyl]piperidine-1-carboxylate (1.3 g, 2.2 mmol) was added HBr/AcOH (2.23 mmol, 12 mL). The mixture was then stirred at 25° C. for 2 hrs. On completion, the reaction mixture was filtered and concentrated under reduced pressure to give a residue. The crude product was triturated with petroleum ether (100 mL) at 25° C. for 30 minutes, then filtered to give the title compound (1.4 g, HOAC) as a brown solid. LC-MS (ESI⁺) m/z 450.4 (M+H)⁺.

### (S)-2-(8-(1′-(2-azaspiro[3.3]heptan-6-yl)-[1,4′-bipiperidin]-4-yl)-6,6a,7,8,9,10-hexahydro-5H-pyrazino[1′,2′:4,5]pyrazino[2,3-c]pyridazin-2-yl)phenol (Intermediate NL)

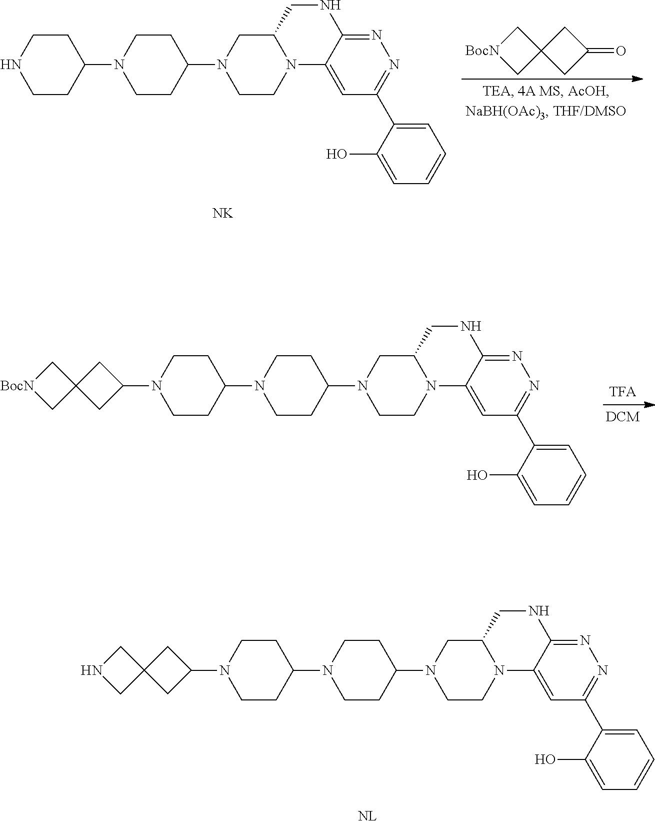 [SMILES: Oc1ccccc1-c1cc2c(nn1)NC[C@H]1CN(C3CCN(C4CCNCC4)CC3)CCN21 ; CC(C)(C)OC(=O)N1CC2(CC(=O)C2)C1 ; "NK" ; CC(C)(C)OC(=O)N1CC2(CC(N3CCC(N4CCC(N5CCN6c7cc(-c8ccccc8O)nnc7NC[C@H]6C5)CC4)CC3)C2)C1 ; Oc1ccccc1-c1cc2c(nn1)NC[C@H]1CN(C3CCN(C4CCN(C5CC6(CNC6)C5)CC4)CC3)CCN21]

Step 1-(S)-tert-butyl 6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)-2-azaspiro[3.3]heptane-2-carboxylate. To a solution of 2-[(10S)-12-[1-(4-piperidyl)-4-piperidyl]-1,5,6,8,12-pentazatricyclo[8.4.0.0^{2,7}]tetradeca-2,4,6-trien-4-yl]phenol (600 mg, 1.18 mmol, HOAC, Intermediate NK) in THF (6 mL) and DMSO (2 mL) was added 4 Å molecular sieves (600 mg) and TEA (357 mg, 3.53 mmol) and the mixture was stirred for 0.5 hr. Then tert-butyl 6-oxo-2-azaspiro[3.3]heptane-2-carboxylate (323 mg, 1.53 mmol, CAS #1147557-97-8) and AcOH (282 mg, 4.71 mmol) was added and the mixture was stirred for 2 hrs. Then NaBH(OAc)₃ (748 mg, 3.53 mmol) was added at 0° C. and the mixture was stirred at 25° C. for 0.5 hr. On completion, the reaction mixture was filtered and concentrated under reduced pressure to give a residue. The residue was purified by prep-HPLC (NH₃·H₂O condition) to give the title compound (200 mg, 20% yield) as a yellow solid. LC-MS (ESI⁺) m/z 645.5 (M+H)⁺.

Step 2-(S)-2-(8-(1′-(2-azaspiro[3.3]heptan-6-yl)-[1,4′-bipiperidin]-4-yl)-6,6a,7,8,9,10-hexahydro-5H-pyrazino[1′,2′:4,5]pyrazino[2,3-c]pyridazin-2-yl)phenol. To a solution of tert-butyl 6-[4-[4-[(10S)-4-(2-hydroxyphenyl)-1,5,6,8,12-pentazatricyclo[8.4.0.0^{2,7}]tetradeca-2,4,6-trien-12-yl]-1-piperidyl]-1-piperidyl]-2-azaspiro[3.3]heptane-2-carboxylate (200 mg, 310 umol) in DCM (2 mL) was added TFA (1.54 g, 13.5 mmol). The mixture was then stirred at 25° C. for 1 hr. On completion, the reaction mixture was filtered and concentrated under reduced pressure to give a residue. The residue was purified by prep-HPLC (FA condition) to give the title compound (180 mg, 85% yield, FA) as a red solid. LC-MS (ESI⁺) m/z 545.5 (M+H)⁺.

### Tert-butyl 3-formylpiperidine-1-carboxylate (Intermediate NM)

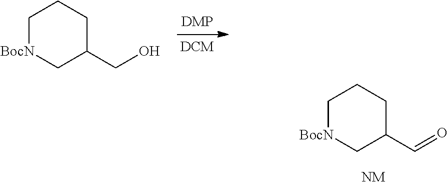 [SMILES: CC(C)(C)OC(=O)N1CCCC(CO)C1 ; CC(C)(C)OC(=O)N1CCCC(C=O)C1]

To a solution of tert-butyl (3R)-3-(hydroxymethyl) piperidine-1-carboxylate (500 mg, 2 mmol, CAS #116574-71-1) in DCM (5 mL) was added DMP (2.46 g, 5.81 mmol). The mixture was stirred at 25° C. for 30 mins. The reaction mixture was quenched with Na₂CO₃ (10) mL and Na₂SO₃ (10 mL) at 25° C., and then diluted with DCM (10 mL) and extracted with DCM (10 mL×3). The combined organic layers were dried over Na₂SO₄, filtered and concentrated under reduced pressure to give a residue. The residue was purified by column chromatography (SiO₂, Petroleum ether/Ethyl acetate=20/1 to 10/1) to give the title compound (400 mg, 80% yield) as a white oil.

### Tert-butyl 3-((4-((S)-2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)methyl)piperidine-1-carboxylate (Intermediate NN)

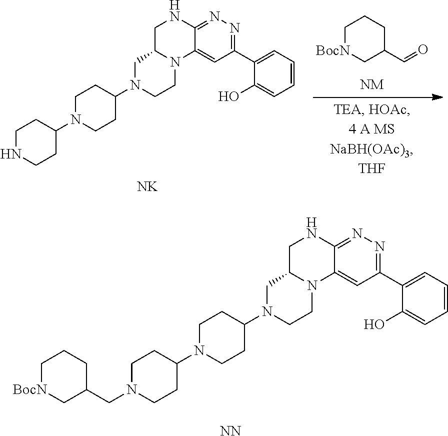 [SMILES: Oc1ccccc1-c1cc2c(nn1)NC[C@H]1CN(C3CCN(C4CCNCC4)CC3)CCN21 ; CC(C)(C)OC(=O)N1CCCC(C=O)C1 ; "NK" ; CC(C)(C)OC(=O)N1CCCC(CN2CCC(N3CCC(N4CCN5c6cc(-c7ccccc7O)nnc6NC[C@H]5C4)CC3)CC2)C1 ; "NN"]

A mixture of tert-butyl (3R)-3-formylpiperidine-1-carboxylate (400 mg, 1.88 mmol, Intermediate NM), 2-[(10S)-12-[1-(4-piperidyl)-4-piperidyl]-1,5,6,8,12-pentazatricyclo[8.4.0.0^{2,7}]tetradeca-2,4,6-trien-4-yl]phenol (380 mg, 781 umol, HCl, Intermediate NK), HOAc (188 mg, 3.13 mmol), KOAc (230 mg, 2.34 mmol) and NaBH(OAc)₃ (414 mg, 1.95 mmol), 4 Å molecular sieves (600 mg) in THF (0.5 mL), DMSO (0.5 mL) was degassed and purged with N₂ three times. Then the mixture was stirred at 25° C. for 12 hours under N₂ atmosphere. The crude product was purified by reversed-phase (FA condition) to give the title compound (500 mg, 91% yield, FA) as a white solid. LC-MS (ESI⁺) m/z 647.4 (M+H)⁺.

### 2-((S)-8-(1′-((R)-piperidin-3-ylmethyl)-[1,4′-bipiperidin]-4-yl)-6,6a,7,8,9,10-hexahydro-5H-pyrazino[1′,2′:4,5]pyrazino[2,3-c]pyridazin-2-yl)phenol (Intermediate NO) and 2-((S)-8-(1′-((S)-piperidin-3-ylmethyl)-[1,4′-bipiperidin]-4-yl)-6,6a,7,8,9,10-hexahydro-5H-pyrazino[1′,2′:4,5]pyrazino[2,3-c]pyridazin-2-yl)phenol (Intermediate NP)

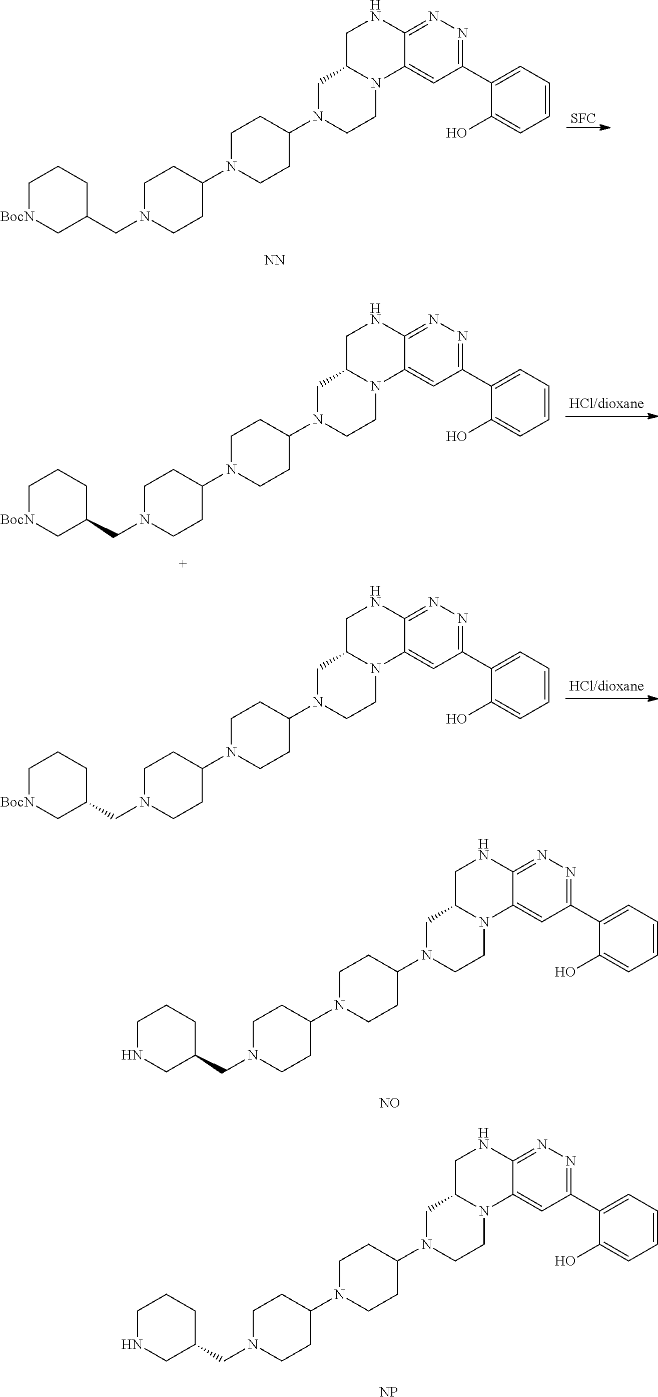

Step 1-tert-butyl (S)-3-((4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)methyl)piperidine-1-carboxylate and tert-butyl (R)-3-((4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)methyl)piperidine-1-carboxylate. Tert-butyl 3-((4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)methyl)piperidine-1-carboxylate (500 mg, 772.96 umol) was separated by SFC (column: DAICEL CHIRALPAK IC(250 mm×3 mm, 10 um); mobile phase: ACN/MeOH (0.1% NH₃H₂O); B %: 58%-58%, A5.2; 103 min) to give tert-butyl (3S)-3-[[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]methyl]piperidine-1-carboxylate (240 mg, 48% yield) as a white solid (LC-MS (ESI⁺) m/z 647.4 (M+H)⁺) and tert-butyl (3R)-3-[[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]methyl]piperidine-1-carboxylate (220 mg, 44% yield) as a white solid (LC-MS (ESI⁺) m/z 647.4 (M+H)⁺).

Step 2-2-((S)-8-(1′-((R)-piperidin-3-ylmethyl)-[1,4′-bipiperidin]-4-yl)-6,6a,7,8,9,10-hexahydro-5H-pyrazino[1′,2′:4,5]pyrazino[2,3-c]pyridazin-2-yl)phenol. To a solution of tert-butyl (3S)-3-[[4-[4-[(10S)-4-(2-hydroxyphenyl)-1,5,6,8,12-pentazatricyclo[8.4.0.0²′7]tetradeca-2,4,6-trien-12-yl]-1-piperidyl]-1-piperidyl]methyl]piperidine-1-carboxylate (100.00 mg, 154.59 umol) in DCM (1 mL) was added HCl/dioxane (8 M, 19.32 uL). The mixture was stirred at 25° C. for 2 hrs. On completion, the reaction mixture filtered and concentrated under reduced pressure to give the title compound (85 mg) as a white solid. LC-MS (ESI⁺) m/z 547.3 (M+H)⁺.

Step 3-2-((S)-8-(1′-((S)-piperidin-3-ylmethyl)-[1,4′-bipiperidin]-4-yl)-6,6a,7,8,9,10-hexahydro-5H-pyrazino[1′,2′:4,5]pyrazino[2,3-c]pyridazin-2-yl)phenol. To a solution of tert-butyl (3R)-3-[[4-[4-[(10S)-4-(2-hydroxyphenyl)-1,5,6,8,12-pentazatricyclo[8.4.0.0^{2,7}]tetradeca-2,4,6-trien-12-yl]-1-piperidyl]-1-piperidyl]methyl]piperidine-1-carboxylate (100.00 mg, 154.59 umol) in DCM (1 mL) was added HCl/dioxane (8 M, 19.32 uL). The mixture was stirred at 25° C. for 2 hrs. The reaction mixture filtered and concentrated under reduced pressure to give the title compound (85 mg, HCl) as a white solid. LC-MS (ESI⁺) m/z 547.3 (M+H)⁺.

### (S)-4-(2-(2-hydroxyphenyl)-6a,7,9,10-tetrahydro-5H-pyrazino[1′,2′:4,5]pyrazino[2,3-c]pyridazin-8(6H)-yl)cyclohexanone (Intermediate NQ)

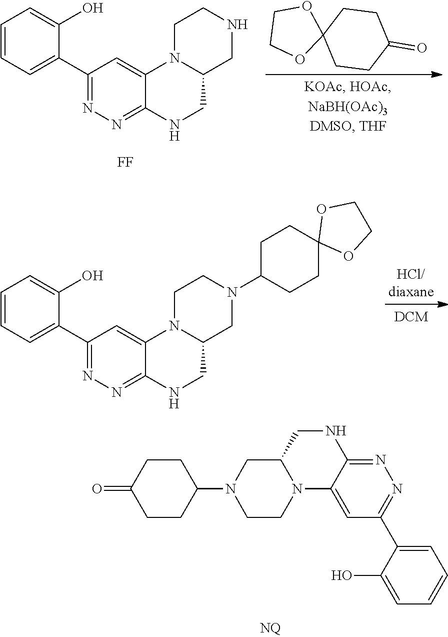

Step 1-(S)-2-(8-(1,4-dioxaspiro[4.5]decan-8-yl)-6,6a,7,8,9,10-hexahydro-5H-pyrazino[1′,2′:4,5]pyrazino[2,3-c]pyridazin-2-yl)phenol. A mixture of 2-[(10R)-1,5,6,8,12-pentazatricyclo[8.4.0.0^{2,7}]tetradeca-2,4,6-trien-4-yl]phenol (4 g, 12.5 mmol, HCl, Intermediate FF), 1,4-dioxaspiro[4.5]decan-8-one (2.93 g, 18.8 mmol, CAS #4746-97-8), KOAc (3.68 g, 37.5 mmol), HOAc (3.00 g, 50.0 mmol), 4 Å molecular sieves (7 g) and NaBH(OAc)₃ (7.95 g, 37.5 mmol) in DMSO (40 mL) was degassed and purged with N₂ three times. Then the mixture was stirred at 25° C. for 12 hrs under N₂ atmosphere. On completion, the reaction mixture was concentrated under reduced pressure to remove water. The residue was diluted with DCM (40 mL) and extracted with DCM (100 mL×3). The combined organic layers were dried over Na₂SO₄, filtered and concentrated under reduced pressure to give a residue. The residue was purified by column chromatography (SiO₂, DCM/Ethyl acetate=1/0 to 1/8) to give the title compound (3.4 g, 59% yield) as a yellow solid. LC-MS (ESI⁺) m/z 424.2 (M+H)⁺.

Step 2-(S)-4-(2-(2-hydroxyphenyl)-6a,7,9,10-tetrahydro-5H-pyrazino[1′,2′:4,5]pyrazino[2,3-c]pyridazin-8(6H)-yl)cyclohexanone. A solution of 2-[(10S)-12-(1,4-dioxaspiro[4.5]decan-8-yl)-1,5,6,8,12-pentazatricyclo[8.4.0.0^{2,7}]tetradeca-2,4,6-trien-4-yl]phenol (1 g, 2 mmol) in HCl (2 M, 20.00 mL) was stirred at 50° C. for 2 hours. On completion, the reaction mixture was quenched with Na₂SO₃ sol. (20 mL) at 25° C., then NaHCO₃ (10 mL) to adjust the pH to 7. Then the mixture was diluted with water (10 mL) and extracted with DCM (20 mL×3). The combined organic layers were dried over Na₂SO₄, filtered and concentrated under reduced pressure to give the title compound (1 g) as a yellow solid. LC-MS (ESI⁺) m/z 380.4 (M+H)⁺.

### (S)-tert-butyl 4-((1-(4-(2-(2-hydroxyphenyl)-6a,7,9,10-tetrahydro-5H-pyrazino[1′,2′:4,5]pyrazino[2,3-c]pyridazin-8(6H)-yl)cyclohexyl)piperidin-4-yl)methyl)piperazine-1-carboxylate (Intermediate NR)

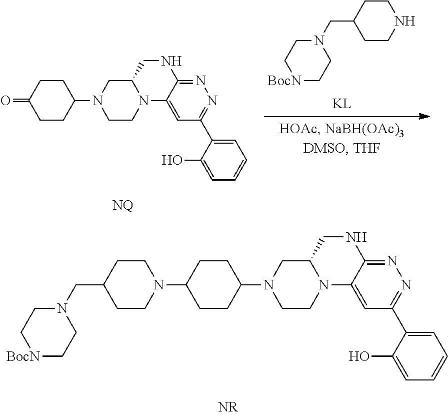 [SMILES: O=C1CCC(N2CCN3c4cc(-c5ccccc5O)nnc4NC[C@H]3C2)CC1 ; CC(C)(C)OC(=O)N1CCN(CC2CCNCC2)CC1 ; CC(C)(C)OC(=O)N1CCN(CC2CCN(C3CCC(N4CCN5c6cc(-c7ccccc7O)nnc6NC[C@H]5C4)CC3)CC2)CC1]

A mixture of 4-[(10S)-4-(2-hydroxyphenyl)-1,5,6,8,12-pentazatricyclo[8.4.0.0^{2,7}]tetradeca-2,4,6-trien-12-yl]cyclohexanone (1 g, 3 mmol, Intermediate NQ), tert-butyl 4-(4-piperidylmethyl)piperazine-1-carboxylate (1.12 g, 3.95 mmol, Intermediate KL), HOAc (475 mg, 7.91 mmol), NaBH(OAc)₃ (1.40 g, 6.59 mmol) and 4 Å molecular sieves (2 g) in THF (5 mL), DMSO (5 mL) was degassed and purged with N₂ three times. Then the mixture was stirred at 25° C. for 12 hrs under N₂ atmosphere. The crude product was purified by reversed-phase (FA condition) to give the title compound (200 mg, 11% yield) as a white solid. LC-MS (ESI⁺) m/z 647.4 (M+H)⁺.

### Tert-butyl 4-((1-((1R,4s)-4-((S)-2-(2-hydroxyphenyl)-6a,7,9,10-tetrahydro-5H-pyrazino[1′,2′:4,5]pyrazino[2,3-c]pyridazin-8(6H)-yl)cyclohexyl)piperidin-4-yl)methyl)piperazine-1-carboxylate (Intermediate NS) and tert-butyl 4-((1-((1S,4r)-4-((S)-2-(2-hydroxyphenyl)-6a,7,9,10-tetrahydro-5H-pyrazino[1′,2′:4,5]pyrazino[2,3-c]pyridazin-8(6H)-yl)cyclohexyl)piperidin-4-yl)methyl)piperazine-1-carboxylate (Intermediate NT)

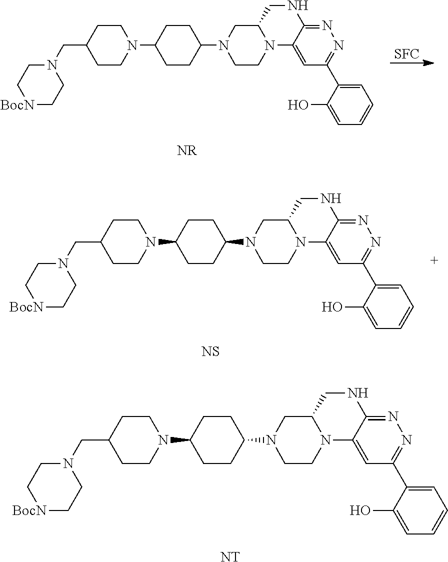 [SMILES: CC(C)(C)OC(=O)N1CCN(CC2CCN(C3CCC(N4CCN5c6cc(-c7ccccc7O)nnc6NC[C@H]5C4)CC3)CC2)CC1 ; "SFC" ; CC(C)(C)OC(=O)N1CCN(CC2CCN([C@H]3CC[C@@H](N4CCN5c6cc(-c7ccccc7O)nnc6NC[C@H]5C4)CC3)CC2)CC1 ; "NS" ; CC(C)(C)OC(=O)N1CCN(CC2CCN([C@H]3CC[C@H](N4CCN5c6cc(-c7ccccc7O)nnc6NC[C@H]5C4)CC3)CC2)CC1 ; "NT"]

(S)-tert-butyl 4-((1-(4-(2-(2-hydroxyphenyl)-6a,7,9,10-tetrahydro-5H-pyrazino[1′,2′:4,5]pyrazino[2,3-c]pyridazin-8(6H)-yl)cyclohexyl)piperidin-4-yl)methyl)piperazine-1-carboxylate (400 mg, FA, Intermediate NR) was purified by SFC (column: DAICEL CHIRALCEL OD (250 mm×50 mm, 10 um); mobile phase: [0.1% NH3H2O ETOH]; B %: 50%-50%, 4.3; 40 min) to give tert-butyl 4-((1-((1R,4s)-4-((S)-2-(2-hydroxyphenyl)-6a,7,9,10-tetrahydro-5H-pyrazino[1′,2′:4,5]pyrazino[2,3-c]pyridazin-8(6H)-yl)cyclohexyl)piperidin-4-yl)methyl)piperazine-1-carboxylate (200 mg, 11% yield) as a white solid (LC-MS (ESI⁺) m/z 647.4 (M+H)⁺) and tert-butyl 4-((1-((1S,4r)-4-((S)-2-(2-hydroxyphenyl)-6a,7,9,10-tetrahydro-5H-pyrazino[1′,2′:4,5]pyrazino[2,3-c]pyridazin-8(6H)-yl)cyclohexyl)piperidin-4-yl)methyl)piperazine-1-carboxylate (200 mg, 10% yield) as a white solid (LC-MS (ESI⁺) m/z 647.4 (M+H)⁺).

### 2-((S)-8-((1s,4R)-4-(4-(piperazin-1-ylmethyl)piperidin-1-yl)cyclohexyl)-6,6a,7,8,9,10-hexahydro-5H-pyrazino[1′,2′:4,5]pyrazino[2,3-c]pyridazin-2-yl)phenol (Intermediate NU)

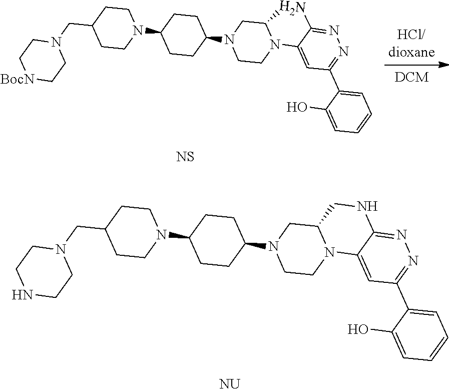 [SMILES: CC(C)(C)OC(=O)N1CCN(CC2CCN([C@H]3CC[C@@H](N4CCN5c6cc(-c7ccccc7O)nnc6N[C@H]5C4)CC3)CC2)CC1 ; "NS" ; Oc1ccccc1-c1cc2c(nn1)NC[C@H]1CN([C@H]3CC[C@@H](N4CCC(CN5CCNCC5)CC4)CC3)CCN21 ; "NU"]

To a solution of tert-butyl 4-[[1-[4-[(10S)-4-(2-hydroxyphenyl)-1,5,6,8,12-pentazatricyclo[8.4.0.0^{2,7}]tetradeca-2,4,6-trien-12-yl]cyclohexyl]-4-piperidyl]methyl]piperazine-1-carboxylate (200 mg, 309 umol, Intermediate NS) in DCM (3 mL) was added HCl/dioxane (4 M, 700 uL). The mixture was stirred at 25° C. for 30 mins. The crude product was purified by reversed-phase (FA condition) to give the title compound (180 mg, 98% yield, FA) as a yellow solid. LC-MS (ESI⁺) m/z 547.4 (M+H)⁺.

### 2-((S)-8-((1r,4S)-4-(4-(piperazin-1-ylmethyl)piperidin-1-yl)cyclohexyl)-6,6a,7,8,9,10-hexahydro-5H-pyrazino[1′,2′:4,5]pyrazino[2,3-c]pyridazin-2-yl)phenol (Intermediate NV)

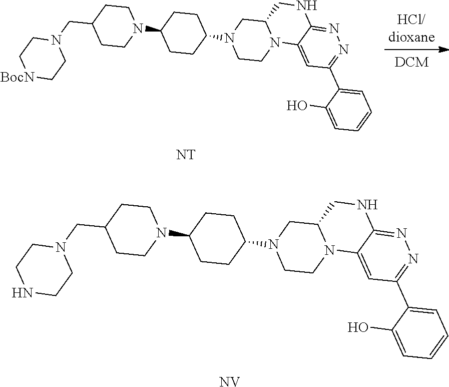 [SMILES: CC(C)(C)OC(=O)N1CCN(CC2CCN([C@H]3CC[C@H](N4CCN5c6cc(-c7ccccc7O)nnc6NC[C@H]5C4)CC3)CC2)CC1 ; "NT" ; Oc1ccccc1-c1cc2c(nn1)NC[C@H]1CN([C@H]3CC[C@H](N4CCC(CN5CCNCC5)CC4)CC3)CCN21 ; "NV"]

To a solution of tert-butyl 4-[[1-[4-[(10S)-4-(2-hydroxyphenyl)-1,5,6,8,12-pentazatricyclo[8.4.0.0^{2,7}]tetradeca-2,4,6-trien-12-yl]cyclohexyl]-4-piperidyl]methyl]piperazine-1-carboxylate (200 mg, 309 umol, Intermediate NT) in DCM (3 mL) was added HCl/dioxane (4 M, 0.7 mL). The mixture was stirred at 25° C. for 30 mins. On completion, the crude product was purified by reversed-phase (FA condition) to give the title compound (180 mg, 98% yield, FA) as a white solid. LC-MS (ESI⁺) m/z 547.4 (M+H)⁺.

### Tert-butyl 2-formylmorpholine-4-carboxylate (Intermediate NW)

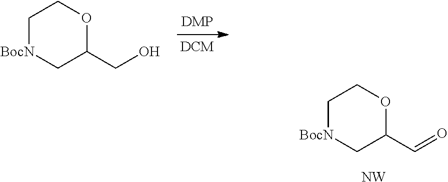

To a solution of tert-butyl 2-(hydroxymethyl) morpholine-4-carboxylate (1 g, 4.60 mmol) in DCM (10 mL) was added DMP (2.34 g, 5.52 mmol, 1.71 mL) at 0° C. The mixture was stirred at 25° C. for 2 hours. On completion, the reaction mixture was concentrated under reduced pressure to give the residue to give the title compound (1 g) as a white oil.

### Tert-butyl (S)-2-((4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)methyl)morpholine-4-carboxylate (Intermediate NX) and tert-butyl (R)-2-((4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)methyl)morpholine-4-carboxylate (Intermediate NY)

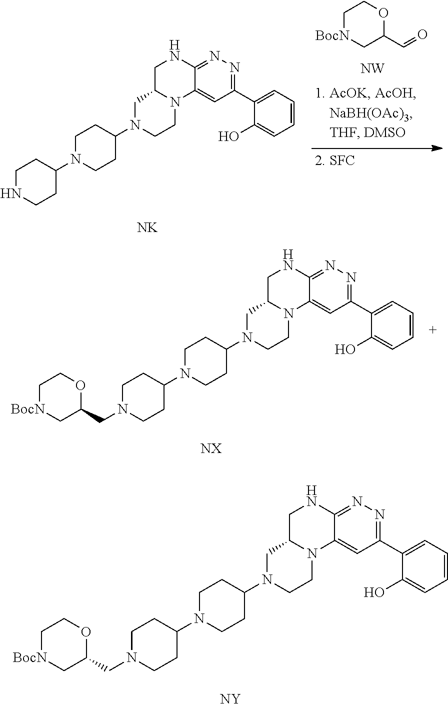

To a solution of 2-[(10S)-12-[1-(4-piperidyl)-4-piperidyl]-1,5,6,8,12-pentazatricyclo[8.4.0.02,7]tetradeca-2,4,6-trien-4-yl]phenol (300 mg, 700 umol, Intermediate NK) in THF (10 mL), DMSO (1 mL) was added AcOK (327 mg, 3.34 mmol) at 25° C. and the mixture was stirred for 0.5 hours. Next, AcOH (120 mg, 2.00 mmol, 114 uL) and tert-butyl 2-formylmorpholine-4-carboxylate (244 mg, 1.13 mmol, Intermediate NW) was added and the mixture was stirred at 25° C. for 1.5 hrs. At last, to the solution was added NaBH(OAc)₃ (424 mg, 2.00 mmol) at 0° C. Then the mixture was stirred at 25° C. for 2 hrs. On completion, the reaction mixture was quenched with H₂O (1 mL) and concentrated under reduced pressure to give the residue. The crude product was purified by reversed-phase HPLC (0.1% FA condition). Then the purified product was purified by reversed-phase HPLC (column: Phenomenex-Cellulose-2 (250 mm×30 mm, 10 um); mobile phase: [ACN/MeOH (0.1% NH3H2O)]; B %: 60%-60%, 10; 150 min) to give tert-butyl (S)-2-((4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)methyl)morpholine-4-carboxylatel (24 mg, 6% yield) as a white solid and tert-butyl (R)-2-((4-((S)-2-(2-hydroxyphenyl)-5,6,6a,7,9,10-hexahydro-8H-pyrazino[1′,2′:4,5]pyrazino[2,3-c]pyridazin-8-yl)-[1,4′-bipiperidin]-1′-yl)methyl)morpholine-4-carboxylate (50 mg, 12% yield) as a white solid. LC-MS (ESI⁺) m/z 649.6 (M+H)⁺.

### 2-((S)-8-(1′-((R)-morpholin-2-ylmethyl)-[1,4′-bipiperidin]-4-yl)-6,6a,7,8,9,10-hexahydro-5H-pyrazino[1′,2′:4,5]pyrazino[2,3-c]pyridazin-2-yl)phenol (Intermediate NZ)

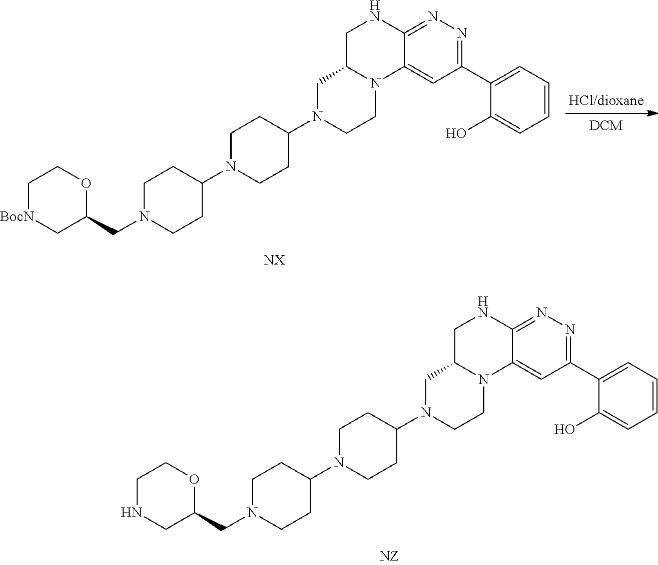 [SMILES: CC(C)(C)OC(=O)N1CCO[C@@H](CN2CCC(N3CCC(N4CCN5c6cc(-c7ccccc7O)nnc6NC[C@H]5C4)CC3)CC2)C1 ; Oc1ccccc1-c1cc2c(nn1)NC[C@H]1CN(C3CCN(C4CCN(C[C@H]5CNCCO5)CC4)CC3)CCN21]

To a solution of tert-butyl (2S)-2-[[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]methyl]morpholine-4-carboxylate (24 mg, 37 umol, Intermediate NX) in HCl/dioxane (1 mL). The mixture was stirred at 25° C. for 2 hrs. On completion, the reaction was concentrated under reduced pressure to give a residue. The residue was purified by prep-HPLC (FA) to give the title compound (15 mg, 53% yield) as a white solid. LC-MS (ESI⁺) m/z 549.3 (M+H)⁺.

### (S)-2-(8-(2-azaspiro[3.3]heptan-6-yl)-6,6a,7,8,9,10-hexahydro-5H-pyrazino[1′,2′:4,5]pyrazino[2,3-c]pyridazin-2-yl)phenol (Intermediate OA)

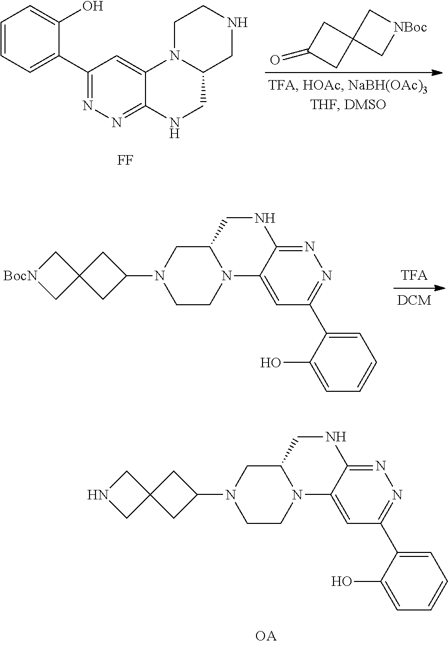

Step 1-(S)-tert-butyl 6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptane-2-carboxylate. To a solution of 2-[(1OR)-1,5,6,8,12-pentazatricyclo[8.4.0.0^{2,7}]tetradeca-2,4,6-trien-4-yl]phenol (4 g, 12.5 mmol, HCl, Intermediate FF) in THF (60 mL) and DMSO (20 mL) was added TEA (2.53 g, 25.0 mmol) and stirred at 25° C. for 30 minutes. Then tert-butyl 6-oxo-2-azaspiro[3.3]heptane-2-carboxylate (5.28 g, 25.0 mmol, CAS #1147557-97-8) and HOAc (2.25 g, 37.5 mmol) were added and the mixture was stirred at 25° C. for 30 minutes. Finally, NaBH(OAc)₃ (7.95 g, 37.5 mmol) was added and the mixture was stirred at 0-25° C. for 12 hrs. The reaction mixture was filtered and concentrated under reduced pressure to give a residue. The crude product was purified by reversed-phase HPLC (0.1% FA condition) to give the title compound (2.5 g, 35% yield FA) as a brown solid. LC-MS (ESI+) m/z 479.5 (M+H)⁺.

Step 2-(S)-2-(8-(2-azaspiro[3.3]heptan-6-yl)-6,6a,7,8,9,10-hexahydro-5H-pyrazino[1′,2′:4,5]pyrazino[2,3-c]pyridazin-2-yl)phenol. To a solution of tert-butyl 6-[(10S)-4-(2-hydroxyphenyl)-1,5,6,8,12-pentazatricyclo[8.4.0.0^{2,7}]tetradeca-2,4,6-trien-12-yl]-2-azaspiro[3.3]heptane-2-carboxylate (500 mg, 1.04 mmol) in DCM (5 mL) was added TFA (1 mL). The mixture was then stirred at 25° C. for 1 hr. On completion, the reaction mixture was concentrated under reduced pressure to give the title compound (1.07 g) as a red oil. LC-MS (ESI⁺) m/z 379.3(M+H)⁺.

### (S)-2-(8-(2-(1-(2-azaspiro[3.3]heptan-6-yl)piperidin-4-yl)-2-azaspiro[3.3]heptan-6-yl)-6,6a,7,8,9,10-hexahydro-5H-pyrazino[1′,2′:4,5]pyrazino[2,3-c]pyridazin-2-yl)phenol (Intermediate OB)

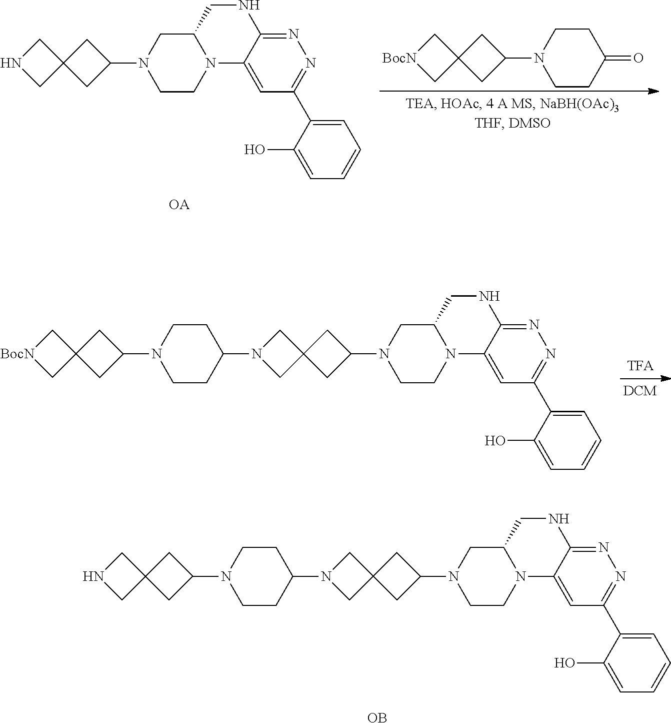 [SMILES: Oc1ccccc1-c1cc2c(nn1)NC[C@H]1CN(C3CC4(CNC4)C3)CCN21 ; CC(C)(C)OC(=O)N1CC2(CC(N3CCC(=O)CC3)C2)C1 ; CC(C)(C)OC(=O)N1CC2(CC(N3CCC(N4CC5(CC(N6CCN7c8cc(-c9ccccc9O)nnc8NC[C@H]7C6)C5)C4)CC3)C2)C1 ; Oc1ccccc1-c1cc2c(nn1)NC[C@H]1CN(C3CC4(C3)CN(C3CCN(C5CC6(CNC6)C5)CC3)C4)CCN21 ; "OB"]

Step 1-(S)-tert-butyl 6-(4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)-2-azaspiro[3.3]heptane-2-carboxylate. To a solution of 2-[(10S)-12-(2-azaspiro[3.3]heptan-6-yl)-1,5,6,8,12-pentazatricyclo[8.4.0.0^{2,7}]tetradeca-2,4,6-trien-4-yl]phenol (400 mg, 812 umol, Intermediate OA) in THF (6 mL) and DMSO (1 mL) was added TEA (109 mg, 1.08 mmol) and stirred the mixture was stirred at 25° C. for 0.5 hr. Then tert-butyl 6-(4-oxo-1-piperidyl)-2-azaspiro[3.3]heptane-2-carboxylate (159 mg, 541 umol, Intermediate MC) and HOAc (97.5 mg, 1.62 mmol) and 4 Å molecular sieves (600 mg, 541 umol) was added and the mixture was stirred at 25° C. for 0.5 hr. Finally, NaBH(OAc)₃ (344 mg, 1.62 mmol) was added and the mixture was stirred at 0-25° C. for 11 hrs. On completion, the pH of reaction mixture was adjusted to greater than 7 with NaHCO₃. The reaction mixture was partitioned between H₂O (20 mL) and ethyl acetate (60 mL). The organic phase was separated, washed with brine (30 mL×2), dried over sodium sulfate, filtered and concentrated under reduced pressure to give a residue. The crude product was purified by reversed-phase HPLC (0.1% FA condition) to give the title compound (140 mg, 35% yield) as a white solid. ¹H NMR (400 MHz, DMSO-d₆) δ=8.22 (s, 1H), 7.90 (d, J=7.2 Hz, 1H), 7.34 (s, 1H), 7.22-7.20 (m, 1H), 6.84 (d, J=8.0 Hz, 1H), 4.04 (d, J=11.6 Hz, 1H), 3.82 (s, 4H), 3.70 (s, 4H), 3.47 (s, 2H), 3.36 (s, 1H), 3.21-3.12 (m, 2H), 2.93-2.85 (m, 2H), 2.85-2.78 (m, 1H), 2.69 (d, J=11.6 Hz, 2H), 2.63-2.57 (m, 4H), 2.52 (d, J=2.0 Hz, 1H), 2.34-2.29 (m, 1H), 2.24-2.20 (m, 4H), 1.95-1.92 (m, 4H), 1.72-1.65 (m, 4H), 1.58-1.51 (m, 1H), 1.35 (s, 9H). LC-MS (ESI⁺) m/z 657.4(M+H)⁺.

Step 2-(S)-2-(8-(2-(1-(2-azaspiro[3.3]heptan-6-yl)piperidin-4-yl)-2-azaspiro[3.3]heptan-6-yl)-6,6a,7,8,9,10-hexahydro-5H-pyrazino[1′,2′:4,5]pyrazino[2,3-c]pyridazin-2-yl)phenol. To a solution of tert-butyl 6-[4-[6-[(10S)-4-(2-hydroxyphenyl)-1,5,6,8,12-pentazatricyclo[8.4.0.0^{2,7}]tetradeca-2,4,6-trien-12-yl]-2-azaspiro[3.3]heptan-2-yl]-1-piperidyl]-2-azaspiro[3.3]heptane-2-carboxylate (90 mg, 128 umol) in DCM (0.7 mL) was added TFA (0.14 mL). The mixture was then stirred at 25° C. for 1 hr. On completion, the reaction mixture was concentrated under reduced pressure to give a residue. The crude product was purified by reversed-phase HPLC (0.1% FA condition) to give the title compound (76 mg, 97% yield) as a pink solid. LC-MS (ESI⁺) m/z 557.4 (M+H)⁺.

### (S)-2-(8-(1-(4-(piperazin-1-ylmethyl)cyclohexyl)piperidin-4-yl)-6,6a,7,8,9,10-hexahydro-5H-pyrazino[1′,2′:4,5]pyrazino[2,3-c]pyridazin-2-yl)phenol (Intermediate OC)

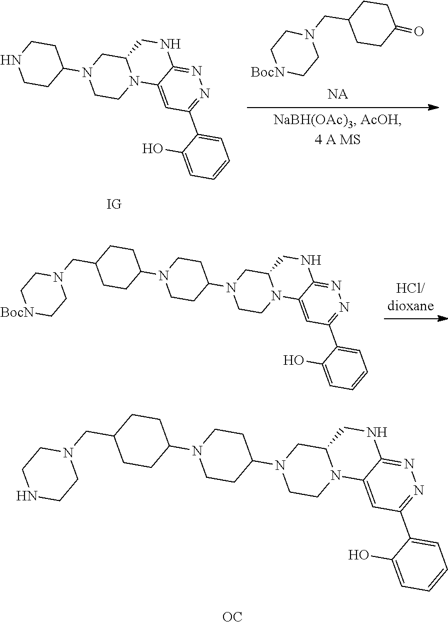 [SMILES: Oc1ccccc1-c1cc2c(nn1)NC[C@H]1CN(C3CCNCC3)CCN21 ; CC(C)(C)OC(=O)N1CCN(CC2CCC(=O)CC2)CC1 ; CC(C)(C)OC(=O)N1CCN(CC2CCC(N3CCC(N4CCN5c6cc(-c7ccccc7O)nnc6NC[C@H]5C4)CC3)CC2)CC1 ; Oc1ccccc1-c1cc2c(nn1)NC[C@H]1CN(C3CCN(C4CCC(CN5CCNCC5)CC4)CC3)CCN21 ; "OC"]

Step 1-(S)-tert-butyl 4-((4-(4-(2-(2-hydroxyphenyl)-6a,7,9,10-tetrahydro-5H-pyrazino[1′,2′:4,5]pyrazino[2,3-c]pyridazin-8(6H)-yl)piperidin-1-yl)cyclohexyl)methyl)piperazine-1-carboxylate. To a solution of 2-[(10S)-12-(4-piperidyl)-1,5,6,8,12-pentazatricyclo[8.4.0.02,7]tetradeca-2,4,6-trien-4-yl]phenol (300 mg, 727.30 umol, FA, Intermediate IG) in THF (4 mL) and DMSO (4 mL) was added TEA (147 mg, 1.45 mmol), AcOH (87.3 mg, 1.45 mmol), 4 Å molecular sieves (0.1 g) and tert-butyl 4-[(4-oxocyclohexyl)methyl]piperazine-1-carboxylate (431 mg, 1.45 mmol, Intermediate NA). The mixture was then stirred at 40° C. for 2 h. Then NaBH(OAc)₃ (462 mg, 2.18 mmol) was added at 0° C., and the mixture was stirred at 25° C. for 12 h. On completion, the reaction mixture was filtered and concentrated under reduced pressure to give a residue. The crude product was purified by reversed-phase HPLC (0.1% FA condition). After prep. HPLC purification, the eluent was concentrated to remove organic solvents. The residual aqueous solution was lyophilized to give the title compound (0.4 g, 78% yield, FA) a yellow solid. LC-MS (ESI⁺) m/z 547.4 (M+H)⁺.

Step 2-(S)-2-(8-(1-(4-(piperazin-1-ylmethyl)cyclohexyl)piperidin-4-yl)-6,6a,7,8,9,10-hexahydro-5H-pyrazino[1′,2′:4,5]pyrazino[2,3-c]pyridazin-2-yl)phenol. To a solution of tert-butyl 4-[[4-[4-[(10S)-4-(2-hydroxyphenyl)-1,5,6,8,12-pentazatricyclo[8.4.0.02,7]tetradeca-2,4,6-trien-12-yl]-1-piperidyl]cyclohexyl]methyl]piperazine-1-carboxylate (0.3 g, 500 umol) was added HCl/dioxane (8 M, 10 mL). The mixture was stirred at 25° C. for 1 hr. On completion, the reaction mixture was concentrated under reduced pressure to give a residue. The crude product was purified by reversed-phase HPLC (0.1% FA condition). After prep. HPLC purification, the eluent was concentrated to remove organic solvents. The residual aqueous solution was lyophilized to give a yellow solid (0.2 g, 72% yield, FA). LC-MS (ESI+) m/z 547.5 (M+H)⁺.

### 2-((S)-8-(1′-((R)-morpholin-2-ylmethyl)-[1,4′-bipiperidin]-4-yl)-6,6a,7,8,9,10-hexahydro-5H-pyrazino[1′,2′:4,5]pyrazino[2,3-c]pyridazin-2-yl)phenol (Intermediate OD)

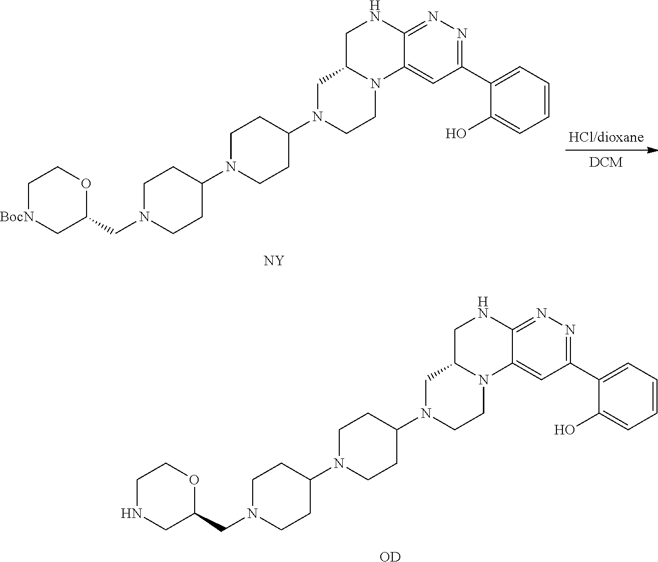 [SMILES: CC(C)(C)OC(=O)N1CCO[C@H](CN2CCC(N3CCC(N4CCN5c6cc(-c7ccccc7O)nnc6NC[C@H]5C4)CC3)CC2)C1 ; "NY" ; Oc1ccccc1-c1cc2c(nn1)NC[C@H]1CN(C3CCN(C4CCN(C[C@H]5CNCCO5)CC4)CC3)CCN21 ; "OD"]

To a solution of tert-butyl (2R)-2-[[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]methyl]morpholine-4-carboxylate (50 mg, 77.1 umol, Intermediate NY) in HCl/dioxane (1 mL). The mixture was stirred at 25° C. for 2 hrs. On completion, the reaction mixture was concentrated under reduced pressure. The crude product was purified by reversed-phase HPLC (FA) to give the residue to give the title compound (35 mg, 76% yield) as a white solid. LC-MS (ESI⁺) m/z 549.4 (M+H)⁺.

### (4-ethynyl-2-(3-methoxypropoxy)phenyl)methanamine (Intermediate OF)

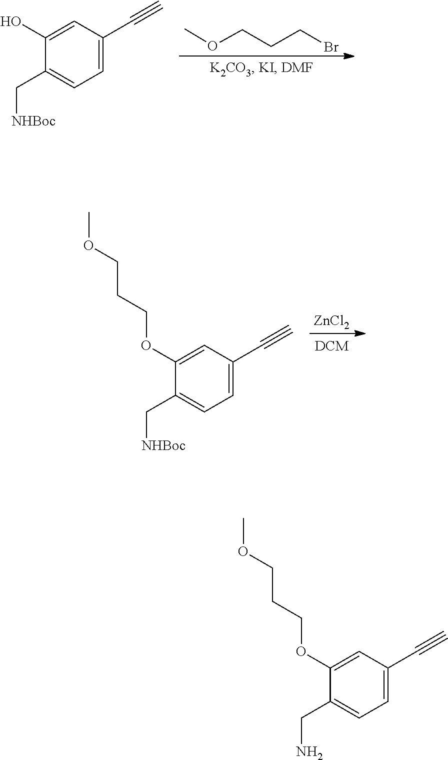

Step 1-Tert-butyl 4-ethynyl-2-(3-methoxypropoxy)benzylcarbamate. To a solution of tert-butyl N-[(4-ethynyl-2-hydroxy-phenyl)methyl]carbamate (1.5 g, 6.1 mmol, synthesized via Steps 1-3 of Intermediate HY) in DMF (35 mL) was added K₂CO₃ (2.51 g, 18.2 mmol), KI (201 mg, 1.21 mmol) and 1-bromo-3-methoxy-propane (1.39 g, 9.10 mmol). The mixture was then stirred at 70° C. for 2 hours. On completion, the reaction mixture was partitioned between EA (100 mL) and H₂O (30 mL). The organic phase was separated, washed with NaCl (20 mL×3), dried over Na₂SO₄, filtered and concentrated under reduced pressure to give a residue. The residue was purified by column chromatography (SiO₂, Petroleum ether/Ethyl acetate=40/1 to Y=10/1) to give the title compound (2.6 g, 61% yield) as a white solid. LC-MS (ESI⁺) m/z 220.1 (M+H)⁺.

Step 2-(4-Ethynyl-2-(3-methoxypropoxy)phenyl)methanamine. To a solution of tert-butyl N-[[4-ethynyl-2-(3-methoxypropoxy)phenyl]methyl]carbamate (2.6 g, 8.1 mmol) in DCM (26 mL) was added ZnCl₂ (2.22 g, 16.3 mmol, 762 uL). The mixture was stirred at 25° C. for 12 hrs. On completion, the reaction mixture was partitioned between H₂O (30 mL) and EA (40 mL). The organic phase was separated, washed with NaCl (10 mL×3), dried over Na₂SO₄, filtered and concentrated under reduced pressure to give a residue. The residue was purified by column chromatography (SiO₂, Petroleum ether/Ethyl acetate=1/1 to 1/5) to give the title compound (3.7 g) as a white solid. LC-MS (ESI⁺) m/z 203.2(M-NH₂+H)⁺.

### Phenyl ((S)-1-((2S,4R)-2-((4-ethynyl-2-(3-methoxypropoxy)benzyl)carbamoyl)-4-hydroxypyrrolidin-1-yl)-3,3-dimethyl-1-oxobutan-2-yl)carbamate (Intermediate OG)

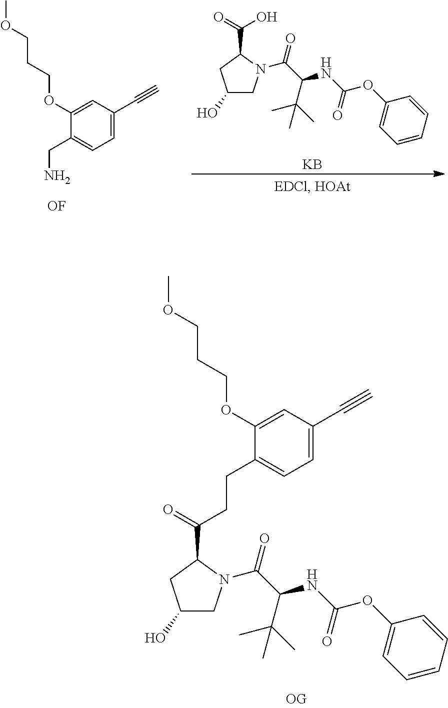

To a solution of (2S,4R)-1-[(2S)-3,3-dimethyl-2-(phenoxycarbonylamino)butanoyl]-4-hydroxy-pyrrolidine-2-carboxylic acid (1 g, 3 mmol, Intermediate KB) in DMSO (25 mL) was added EDCI (1.05 g, 5.49 mmol), HOAt (747 mg, 5.49 mmol, 768 uL) and DIEA (1.06 g, 8.23 mmol, 1.43 mL). After 10 min, [4-ethynyl-2-(3-methoxypropoxy)phenyl]methanamine (1.20 g, 5.49 mmol, Intermediate OF) was added and the mixture was stirred at 25° C. for 12 hrs. On completion, the reaction mixture was partitioned between EA (50 mL) and H₂O (30 mL). The organic phase was separated, washed with NaCL (20 mL×3), dried over Na₂SO₄, filtered and concentrated under reduced pressure to give a residue. The residue was purified by column chromatography (SiO₂, Petroleum ether/Ethyl acetate=10/1 to 1/5) to give the title compound (1.2 g, 66% yield) as a brown solid. LC-MS (ESI+) m/z566.4 (M+H)⁺.

### (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylate (Intermediate MN)

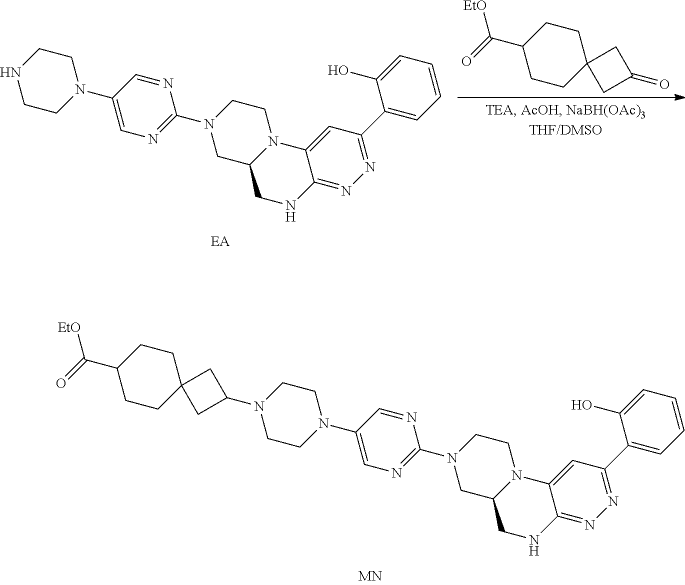

To a solution of 2-[(10S)-12-(5-piperazin-1-ylpyrimidin-2-yl)-1,5,6,8,12-pentazatricyclo[8.4.0.0^{2,7}]tetradeca-2,4,6-trien-4-yl]phenol (21 g, 43.5 mmol, Intermediate EA) in THF (370 mL) and DMSO (180 mL) was added TEA (11.0 g, 108 mmol) and stirred for 0.5 hour. Then ethyl 2-oxospiro[3.5]nonane-7-carboxylate (9.16 g, 43.5 mmol, CAS #1615656-09-1) and AcOH (6.54 g, 108 mmol) was added and stirred for 1.5 hrs. Then NaBH(OAc)₃ (27.7 g, 130 mmol) was added at 0° C. The mixture was stirred at 25° C. for 2 hrs. On completion, the reaction mixture was extracted with DCM (300 mL×3). The combined organic layers were washed with brine (100 mL×2), dried over Na₂SO₄, filtered and concentrated under reduced pressure to give a residue. The crude product was purified by re-crystallization from Petroleum ether: Ethyl acetate 1: 1 (400 mL) at 25° C. Then stirred at 80° C. with 1M FA (200 mL) for 2 hours and give a residue under freezedry. The title compound (13 g, 41% yield) was obtained as av off-pink solid. LC-MS (ESI⁺) m/z 640.3 (M−H)⁺.

### (S)-2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylic acid (Intermediate OH) and (S)-2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylic acid (Intermediate OE)

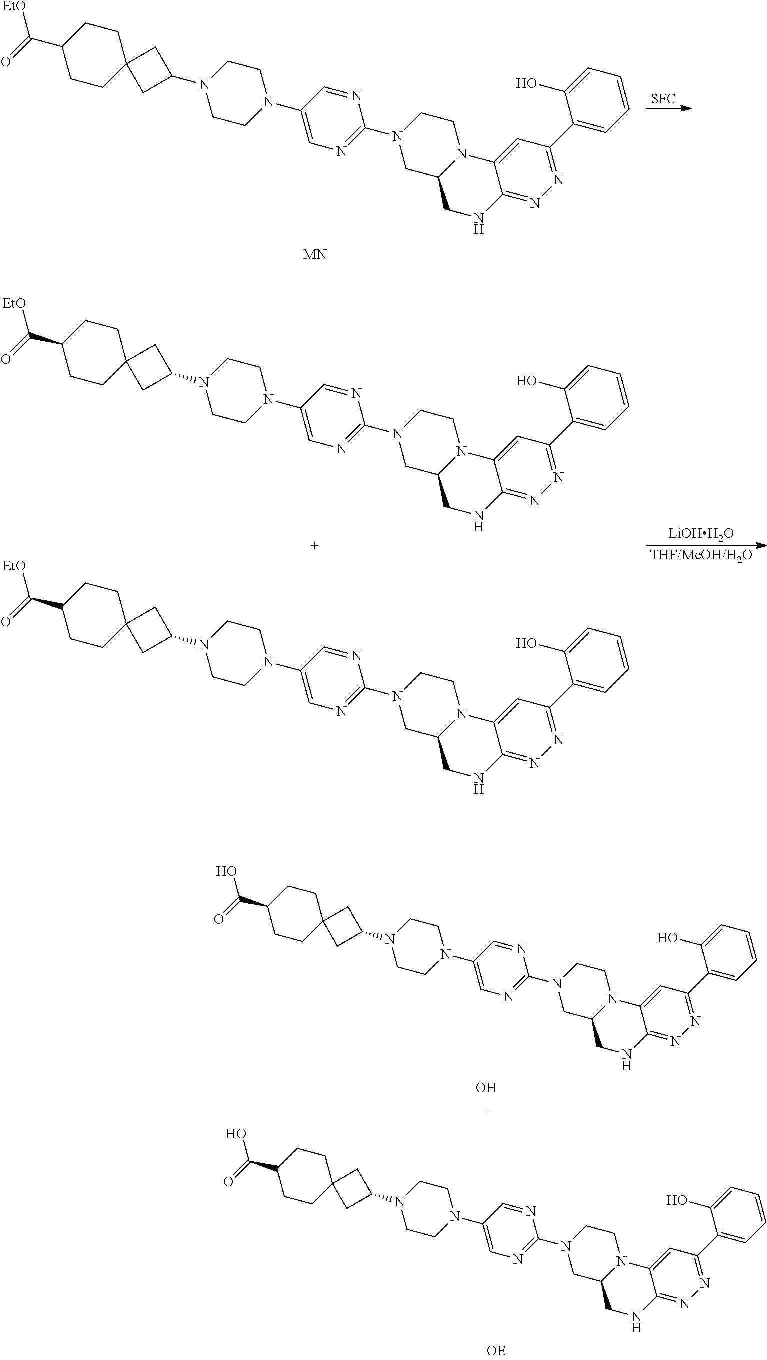

Step 1-(S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylate and (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylate. (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylate (Intermediate M) was separated by SFC (column: DAICEL CHIRALPAK IA(250 mm*30 mm, 10 um); mobile phase: [Hexane-EtOH (0.1% NH₃.H₂O)]; B %: 54%-54%, 20 min) to give (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylate (6 g, 46% yield) as a yellow solid (LC-MS (ESI⁺) m/z 640.3 (M−H)⁺) and (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylate (3.5 g, 27% yield) as a yellow solid. LC-MS (ESI⁺) m/z 640.4 (M−H)⁺.

Step 2-(S)-2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylic acid. To a solution of (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylate (170 mg, 265 umol) in THF (0.5 mL), MeOH (0.5 mL) and H₂O (0.5 mL) was added LiOH·H₂O (55.7 mg, 1.33 mmol). The mixture was stirred at 40° C. for 1 hr. On completion, the reaction mixture was filtered and concentrated under reduced pressure to give a residue. The residue was purified by prep-HPLC (FA condition) to give the title compound (50 mg, 28% yield, FA) as a white solid. LC-MS (ESI⁺) m/z 612.6 (M−H)⁺.

Step 3-(S)-2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylic acid. To a solution of (S)-ethyl 2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperazin-1-yl)spiro[3.5]nonane-7-carboxylate(230 mg, 359 umol) in THF (1 mL), MeOH (1 mL) and H₂O (1 mL) was added LiOH·H₂O (75.4 mg, 1.80 mmol). The mixture was stirred at 25° C. for 1 hour. On completion, the reaction mixture was filtered and concentrated under reduced pressure to give a residue. The residue was purified by prep-HPLC (FA condition) to give the title compound (40 mg, 16% yield, FA) as a white solid. LC-MS (ESI⁺) m/z 612.5 (M−H)⁺.

### Ethyl 2-(4,4-dihydroxypiperidin-1-yl)spiro[3.5]nonane-7-carboxylate (Intermediate OI)

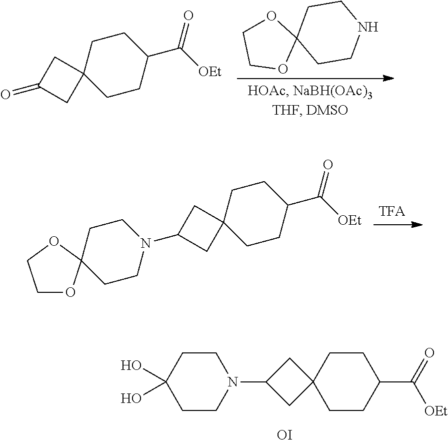

Step 1-Ethyl 2-(1,4-dioxa-8-azaspiro[4.5]decan-8-yl)spiro[3.5]nonane-7-carboxylate. To a solution of ethyl 2-oxospiro[3.5]nonane-7-carboxylate (5 g, 23.8 mmol, CAS #1615656-09-1) in THF (50 mL) and DMSO (10 mL) was added HOAc (4.28 g, 71.3 mmol) and 1,4-dioxa-8-azaspiro[4.5]decane (3.40 g, 23.8 mmol, CAS #177-11-7) and was stirred at 25° C. for 30 minutes. Then NaBH(OAc)₃ (15.1 g, 71.3 mmol) was added and the mixture was stirred at 0-25° C. for 12 hrs. The reaction mixture was quenched with NaHCO₃ (50 mL) at 25° C., and then diluted with ethyl acetate (100 mL) and extracted with water (40 mL×2). The combined organic layers were washed with brine (40 mL×2), dried over Na₂SO₄, filtered and concentrated under reduced pressure to give a residue. The residue was purified by column chromatography (SiO₂, Petroleum ether/Ethyl acetate=10/1 to 1/1) to give the title compound (7 g, 85% yield) as a yellow oil. LC-MS (ESI+) m/z 338.5 (M+H)⁺.

Step 2-ethyl 2-(4,4-dihydroxypiperidin-1-yl)spiro[3.5]nonane-7-carboxylate. To a solution of ethyl 2-(1,4-dioxa-8-azaspiro[4.5]decan-8-yl)spiro[3.5]nonane-7-carboxylate (3 g, 9 mmol) was added in TFA (30 mL). The mixture was then stirred at 60° C. for 12 hrs. On completion, the reaction mixture was filtered and concentrated under reduced pressure to give the title compound (4 g, TFA) as a brown oil. LC-MS (ESI+) m/z 312.3 (M+H)⁺.

### (S)-2-(4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)spiro[3.5]nonane-7-carboxylic acid (Intermediate OJ)

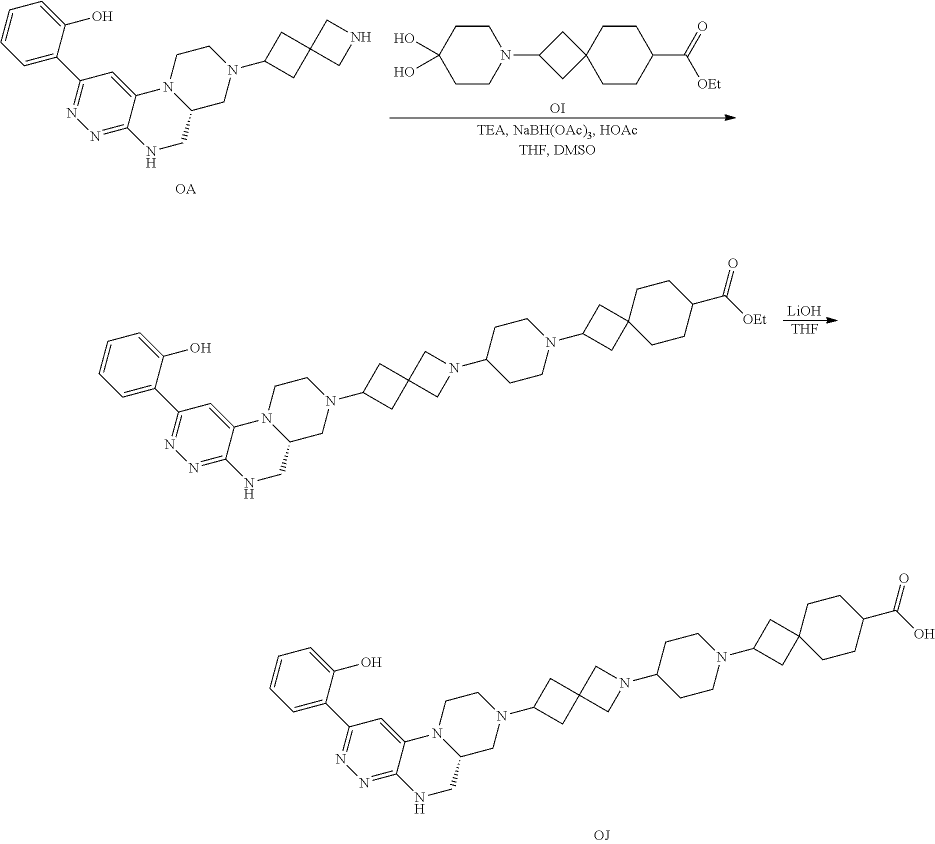

Step 1-(S)-ethyl 2-(4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)spiro[3.5]nonane-7-carboxylate. To a solution of 2-[(10S)-12-(2-azaspiro[3.3]heptan-6-yl)-1,5,6,8,12-pentazatricyclo[8.4.0.0^{2,7}]tetradeca-2,4,6-trien-4-yl]phenol (2 g, 4.06 mmol, TFA, Intermediate OA) in THF (30 mL) and DMSO (6 mL) was added TEA (822 mg, 8.12 mmol) and stirred at 25° C. for 30 minutes. Then ethyl 2-(4,4-dihydroxy-1-piperidyl)spiro[3.5]nonane-7-carboxylate (1.73 g, 4.06 mmol, TFA, Intermediate OI) and HOAc (732 mg, 12.2 mmol) was added and the mixture was stirred at 25° C. for 30 minutes. Finally, NaBH(OAc)₃ (2.58 g, 12.2 mmol) was added and the mixture was stirred at 0-25° C. for 12 hrs. The reaction mixture was filtered and concentrated under reduced pressure to give a residue. The crude product was purified by reversed-phase HPLC (0.1% FA condition) to give the title compound (1 g, 29% yield, FA) as a yellow solid. LC-MS (ESI+) m/z 656.4 (M+H)⁺.

Step 2-(S)-2-(4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)spiro[3.5]nonane-7-carboxylic acid. To a solution of ethyl 2-[4-[6-[(10S)-4-(2-hydroxyphenyl)-1,5,6,8,12-pentazatricyclo[8.4.0.0^{2,7}]tetradeca-2,4,6-trien-12-yl]-2-azaspiro[3.3]heptan-2-yl]-1-piperidyl]spiro[3.5]nonane-7-carboxylate (1 g, 1.52 mmol) in THF (0.5 mL) was added LiOH (2 M, 5.00 mL). The mixture was stirred at 25° C. for 2 hrs. On completion, the reaction mixture was concentrated under reduced pressure to give a residue. The crude product was purified by reversed-phase HPLC (0.1% FA condition) to give a title compound (400 mg, 33% yield, FA) as a yellow solid. LC-MS (ESI+) m/z 628.5 (M+H)⁺.

### (S)-Methyl 6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylate (Intermediate OK)

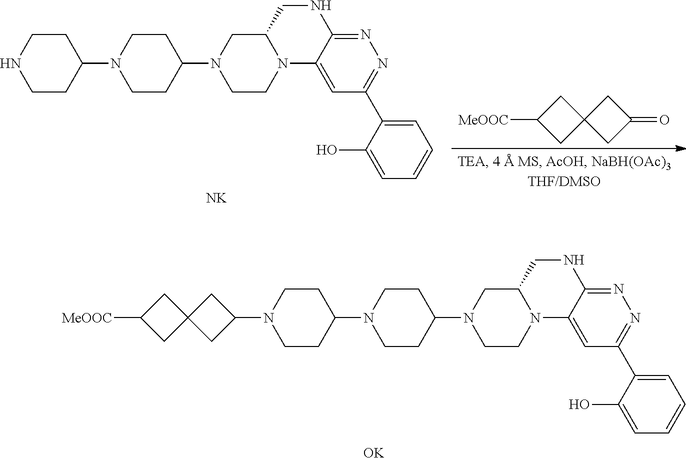 [SMILES: Oc1ccccc1-c1cc2c(nn1)NC[C@H]1CN(C3CCN(C4CCNCC4)CC3)CCN21 ; COC(=O)C1CC2(CC(=O)C2)C1 ; "NK" ; COC(=O)C1CC2(C1)CC(N1CCC(N3CCC(N4CCN5c6cc(-c7ccccc7O)nnc6NC[C@H]5C4)CC3)CC1)C2 ; "OK"]

To a solution of 2-[(10S)-12-[1-(4-piperidyl)-4-piperidyl]-1,5,6,8,12-pentazatricyclo[8.4.0.0^{2,7}]tetradeca-2,4,6-trien-4-yl]phenol (3.3 g, 5.40 mmol, 2HBr, Intermediate NK) in THF (60 mL) and DMSO (30 mL) was added TEA (1.09 g, 10.5 mmol, 1.50 mL). After stirring at rt for 10 min, methyl 2-oxospiro[3.3]heptane-6-carboxylate (907.77 mg, 5.40 mmol), HOAc (972.36 mg, 16.19 mmol, 926.06 uL) and 4 Å MS (6.5 g, 5.40 mmol) were added to the mixture which was stirred for 1 hr. Next, NaBH(OAc)₃ (2.86 g, 13.49 mmol) was added at 0° C. and the mixture was stirred at 0-25° C. for 12 hr. On completion, the reaction mixture was filtered and concentrated under reduced pressure to give a residue. The crude product was purified by reversed-phase HPLC(0.1% FA condition) to give the title compound (900 mg, 1.26 mmol, 23% yield, FA) as a white solid. LC-MS (ESI⁺) m/z 602.3(M+H)⁺.

### (S)-6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylic acid (Intermediate OL) and (Intermediate OM)

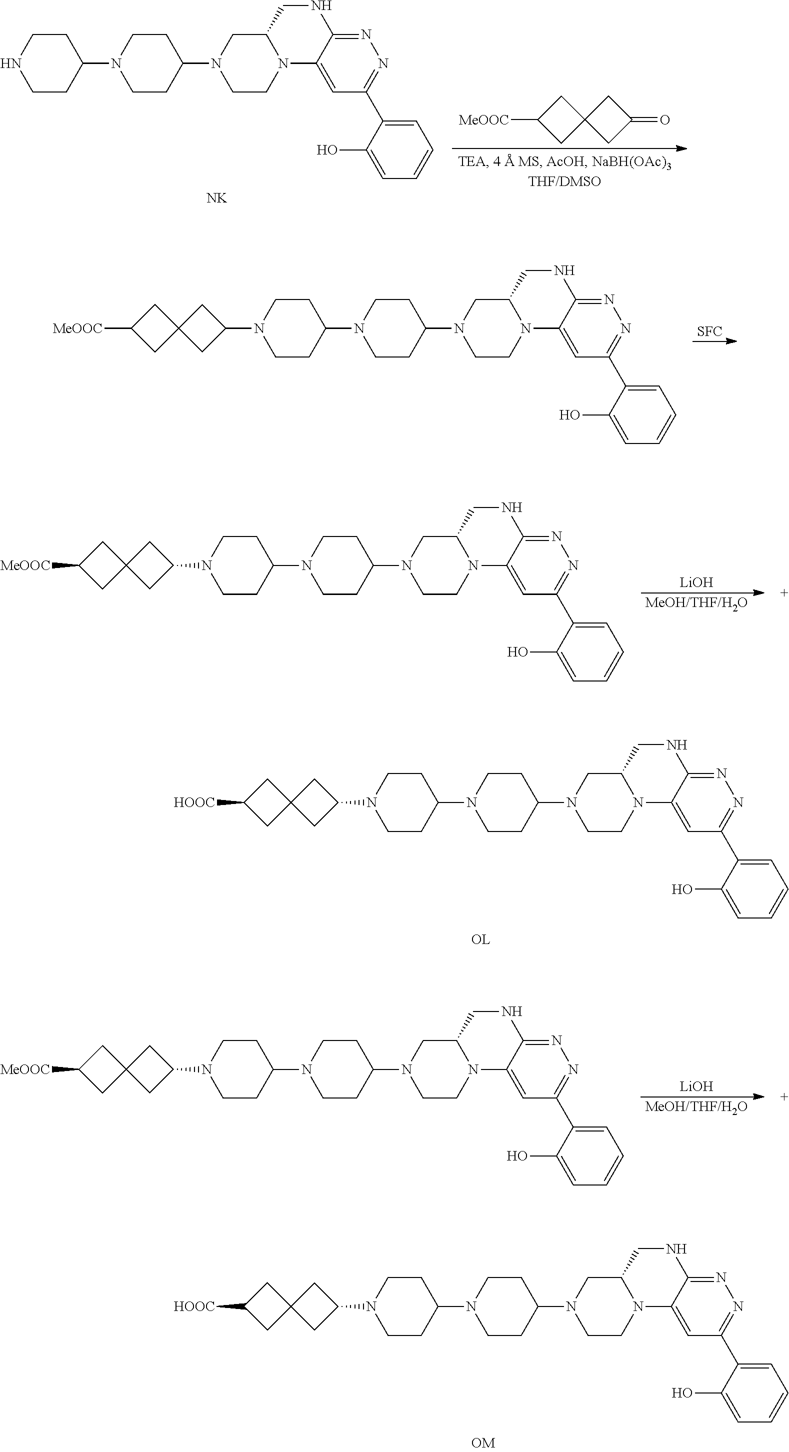

Step 1-(S)-methyl 6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylate. To a solution of 2-[(10S)-12-[1-(4-piperidyl)-4-piperidyl]-1,5,6,8,12-pentazatricyclo[8.4.0.0^{2,7}]tetradeca-2,4,6-trien-4-yl]phenol (3.3 g, 5.40 mmol, 2HBr, Intermediate NK) in THF (60 mL) and DMSO (30 mL) was added TEA (1.09 g, 10.79 mmol, 1.50 mL). After 10 mins of stirring at rt, methyl 2-oxospiro[3.3]heptane-6-carboxylate (907.77 mg, 5.40 mmol, CAS #1138480-98-4), HOAc (972.36 mg, 16.19 mmol, 926.06 uL) and 4 Å molecular sieves (6.5 g, 5.4 mmol) were added and the mixture was stirred for 1 hr at rt. Next, NaBH(OAc)₃ (2.86 g, 13.5 mmol) was added at 0° C., then the mixture was stirred at 0-25° C. for 12 hr. On completion, the reaction mixture was filtered and concentrated under reduced pressure to give a residue. The crude product was purified by reversed-phase HPLC (0.1% FA condition) to give the title compound (900 mg, 23% yield, FA) as a white solid. LC-MS (ESI⁺) m/z 602.3(M+H)⁺

Step 2-(S)-methyl 6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylate and (S)-methyl 6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylate. Compound methyl 2-[4-[4-[(10S)-4-(2-hydroxyphenyl)-1,5,6,8,12-pentazatricyclo[8.4.0.0^{2,7}]tetradeca-2,4,6-trien-12-yl]-1-piperidyl]-1-piperidyl]spiro[3.3]heptane-6-carboxylate (0.9 g, 1.50 mmol) was separated by SFC (column: DAICEL CHIRALPAK AD(250 mm*30 mm,10 um); mobile phase: [ACN/IPA(0.1% NH₃H2O)]; B %: 40%-40%,6.5; 130 min) to give Compound methyl 2-[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]spiro[3.3]heptane-6-carboxylate (400 mg, 41% yield) as a white solid (R^{†}=1.57 min, LC-MS (ESI+) m/z 602.3 (M+H)⁺) and methyl 2-[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]spiro[3.3]heptane-6-carboxylate (410 mg, 45% yield) as a white solid (RT=1.98 min, LC-MS (ESI+) m/z 602.3 (M+H)⁺)

Step 3-(S)-6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylic acid. To a solution of methyl 2-[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]spiro[3.3]heptane-6-carboxylate (400.00 mg, 664.69 umol) in THF (2.5 mL), MeOH (2.5 mL), and H₂O (2.5 mL) was added LiOH·H₂O (139.46 mg, 3.32 mmol). The mixture was stirred at 25° C. for 2 hr. On completion, the crude mixture was purified by reversed-phase HPLC(0.1% FA condition) to give the title compound (320 mg, 74% yield, FA) as a white solid. LC-MS (ESI⁺) m/z 588.3 (M+H)⁺.

Step 4-((S)-6-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)spiro[3.3]heptane-2-carboxylic acid. To a solution of methyl 2-[4-[4-[(10S)-4-(2-hydroxyphenyl)-1,5,6,8,12-pentazatricyclo[8.4.0.02,7]tetradeca-2,4,6-trien-12-yl]-1-piperidyl]-1-piperidyl]spiro[3.3]heptane-6-carboxylate (410.00 mg, 681.31 umol) in THF (2.5 mL), MeOH (2.5 mL), and H₂O (2.5 mL) was added LiOH·H₂O (142.95 mg, 3.41 mmol). The mixture was stirred at 25° C. for 2 hr. The crude mixture was purified by reversed-phase HPLC(0.1% FA condition) to give the title compound (310 mg, 71% yield, FA) as a white solid. LC-MS (ESI⁺) m/z 588.3 (M+H)⁺.

### (S)-7-ethynylchroman-4-amine (Intermediate ON) and (R)-7-ethynylchroman-4-amine (Intermediate OO)

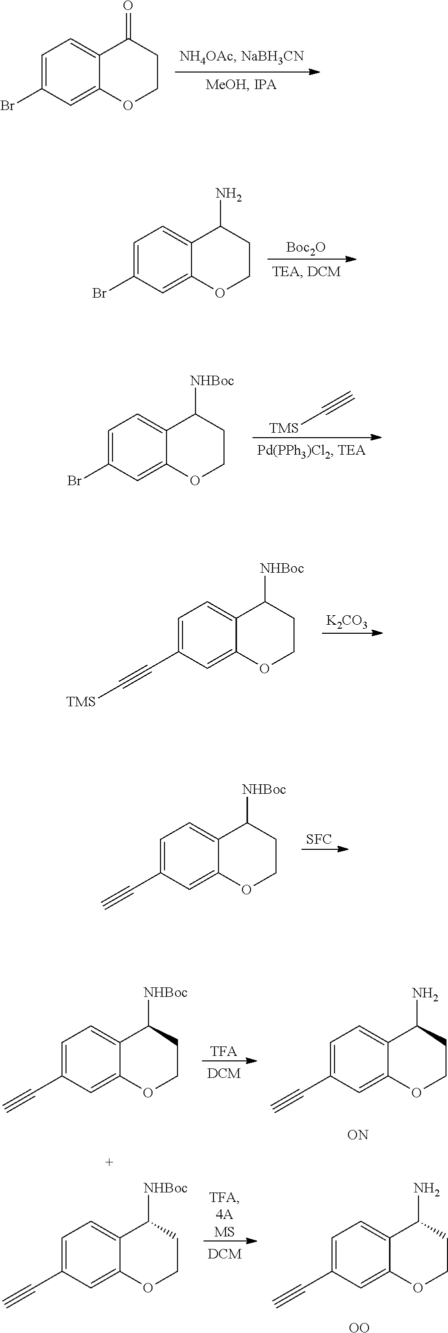

Step 1-7-Bromochroman-4-amine. To a mixture of 7-bromochroman-4-one (2 g, 9 mmol, CAS #18442-22-3) in MeOH (20 mL) and IPA (25 mL) was added NH₄OAc (13.6 g, 176 mmol) and NaBH₃CN (2.77 mg, 44.0 mmol). The mixture was stirred at 25° C. for 4 hrs. Then the temperature was raised to 80° C. and the mixture was stirred 12 hrs. On completion, the reaction mixture was diluted with H₂O (50 mL) and extracted with EA (100 mL×3). The combined organic layers were dried over Na₂SO₄, filtered and concentrated under reduced pressure to give the title compound (3.1 g) as a white solid. LC-MS (ESI+) m/z 212.7 (M+H)⁺.

Step 2-Tert-butyl (7-bromochroman-4-yl)carbamate. To a mixture of 7-bromochroman-4-amine (3.1 g, 14 mmol) in DCM (50 mL) was added Boc₂O (3.26 g, 14.9 mmol) and TEA (4.13 g, 40.8 mmol). The mixture was stirred at 25° C. for 1 hr. On completion, the reaction mixture was diluted with H₂O (50 mL) and extracted with DCM (100 mL×3). The combined organic layers were washed with brine (50 mL×2), dried over Na₂SO₄, filtered and concentrated under reduced pressure to give a residue. The residue was purified by column chromatography (SiO₂,PE: EA=1:0 to 30:1) to give the title compound (2.28 g) as a yellow solid. LC-MS (ESI+) m/z 328.2 (M+H)⁺.

Step 3-Tert-butyl (7-((trimethylsilyl)ethynyl)chroman-4-yl)carbamate. A mixture of tert-butyl (7-bromochroman-4-yl)carbamate (2.28 g, 6.95 umol), ethynyltrimethylsilane (6.82 g, 69.4 mmol), Pd(PPh₃)₂Cl₂ (488 mg, 695 umol) and CuI (265 mg, 1.39 mmol) in TEA (40 mL) was degassed and purged with N₂ three times, and then the mixture was stirred at 85° C. for 7 hrs under N₂ atmosphere. On completion, the reaction mixture was diluted with H₂O (70 mL) and extracted with EA L (70 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=1/0 to 60/1) to give the title compound (2.4 mg, 18% yield) as a brown solid. LC-MS (ESI+) m/z 229.6 (M+H)⁺.

Step 4-Tert-butyl (7-ethynylchroman-4-yl)carbamate. To a mixture of tert-butyl (7-((trimethylsilyl)ethynyl)chroman-4-yl)carbamate (50 mg, 110 umol) in MeOH (1 mL) was added K₃CO₃ (32.9 mg, 329.5 umol). The mixture was stirred at 25° C. for 1 hr. On completion, the reaction mixture was diluted with H₂O (70 mL) and extracted with EA (100 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=1/0 to 60/1) to give the title compound (1.2 g, 59% yield) as a white solid. LC-MS (ESI+) m/z 157.1 (M+H)⁺.

Step 5-(S)-tert-butyl (7-ethynylchroman-4-yl)carbamate and (R)-tert-butyl (7-ethynylchroman-4-yl)carbamate. A mixture of tert-butyl (7-ethynylchroman-4-yl)carbamate (1.2 g, 4.39 mmol) was separated by SFC (column: DAICEL CHIRALPAK IG (250 mm*30 mm,10 um); mobile phase: [0.1% NH₃H₂O MeOH];B %: 35%-35%,5; 70 min) to give (S)-tert-butyl (7-ethynylchroman-4-yl)carbamate (640 mg, 39% yield, LC-MS (ESI+) m/z 157.1 (M+H)⁺) and (R)-tert-butyl (7-ethynylchroman-4-yl)carbamate (450 mg, 36% yield, LC-MS (ESI+) m/z 157.1 (M+H)⁺)

Step 6-(S)-7-ethynylchroman-4-amine. To a mixture of (S)-tert-butyl (7-ethynylchroman-4-yl)carbamate (200 mg, 731.7 mmol) in DCM (4 mL) was added TFA (1.23 g, 10.8 mmol) and 4 Å molecular sieves (200 mg). The mixture was stirred at 25° C. for 2 hr. On completion, filtered and concentrated under reduced pressure to give a residue. The residue was purified by prep-HPLC (FA condition) to give the title compound (95 mg, 54% yield) as a white solid. LC-MS (ESI+) m/z 157.1 (M+H)⁺.

Step 7-(R)-7-ethynylchroman-4-amine. To a solution of tert-butyl N-[(4R)-7-ethynylchroman-4-yl]carbamate (200 mg, 731 umol) in DCM (4 mL) was added 4 Å molecular sieves (0.2 g) and TFA (1.23 g, 10.8 mmol). The mixture was stirred at 25° C. for 0.5 hrs. The reaction mixture was concentrated under reduced pressure to give a residue. The crude product was purified by reversed-phase HPLC (0.10% FA condition). After prep. HPLC purification, the eluent was concentrated to remove organic solvents. The residual aqueous solution was lyophilized to give the title compound as a pink solid (100 mg, 61% yield, FA). LC-MS (ESI⁺) m/z 157.0 (M−NH₂)⁺.

### (2S,4R)-1-((S)-2-(2-chloroacetamido)-3,3-dimethylbutanoyl)-N-(4-ethynylbenzyl)-4-hydroxypyrrolidine-2-carboxamide (Intermediate OP)

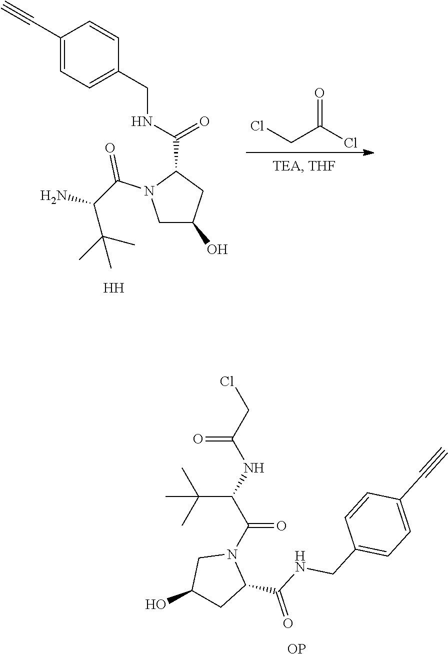

To a mixture of (2S,4R)-1-((S)-2-amino-3,3-dimethylbutanoyl)-N-(4-ethynylbenzyl)-4-hydroxypyrrolidine-2-carboxamide (500 mg, 1.40 mmol, Intermediate HH) in THF (5 mL) was added TEA (283 mg, 2.80 mmol). Then 2-chloroacetyl chloride (205 mg, 1.82 mmol) in THF (5 mL) was added at 0° C. The mixture was stirred at 0-25° C. for 2 hrs. On completion, the reaction mixture was filtered and concentrated under reduced pressure to give a residue. The residue was purified by prep-HPLC (FA condition) to give the title compound (466 mg, 68% yield) as a white solid. LC-MS (ESI⁺) m/z 434.3 (M+H)⁺.

### (2S,4R)-1-((S)-3,3-dimethyl-2-(6-oxohexanamido)butanoyl)-N-(4-ethynylbenzyl)-4-hydroxypyrrolidine-2-carboxamide (Intermediate OQ)

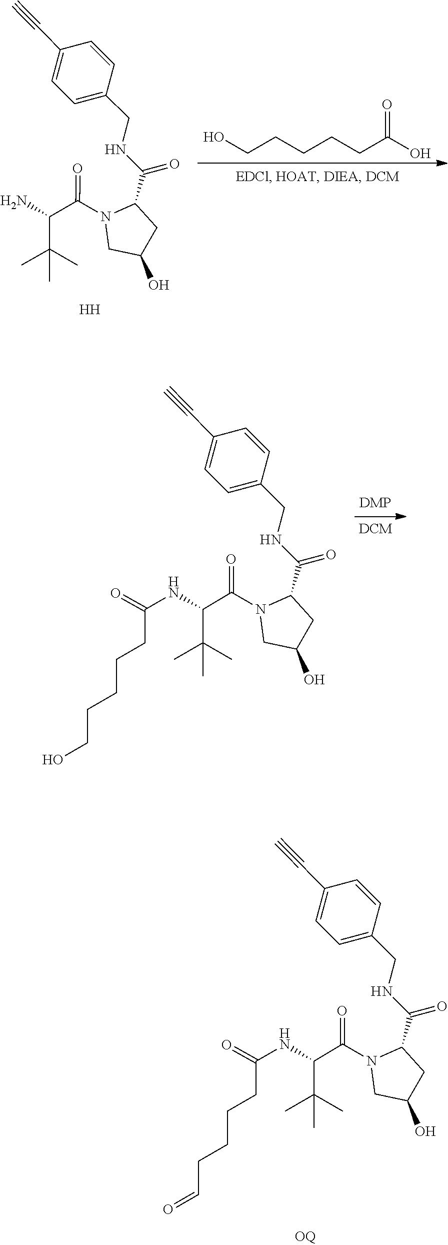

Step 1-(2S,4R)—N-(4-ethynylbenzyl)-4-hydroxy-1-((S)-2-(6-hydroxyhexanamido)-3,3-dimethylbutanoyl)pyrrolidine-2-carboxamide. To a mixture of (2S,4R)-1-((S)-2-amino-3,3-dimethylbutanoyl)-N-(4-ethynylbenzyl)-4-hydroxypyrrolidine-2-carboxamide (400 mg, 1.12 mmol, Intermediate HH), 6-hydroxyhexanoic acid (147 mg, 1.12 mmol, CAS #1191-25-9) in DCM (8 mL) was added DIEA (578 mg, 4.48 mmol), HOAt (183 mg, 1.34 mmol) and EDCI (257 mg, 1.34 mmol). The mixture was stirred at 25° C. for 2 hrs. On completion, the reaction mixture was filtered and concentrated under reduced pressure to give a residue. The residue was purified by prep-HPLC (FA condition) to give the title compound (435 mg, 74% yield) as a white solid. LC-MS (ESI+) m/z 472.1 (M+H)⁺.

Step 2-(2S,4R)-1-((S)-3,3-dimethyl-2-(6-oxohexanamido)butanoyl)-N-(4-ethynylbenzyl)-4-hydroxypyrrolidine-2-carboxamide. To a mixture of (2S,4R)—N-(4-ethynylbenzyl)-4-hydroxy-1-((S)-2-(6-hydroxyhexanamido)-3,3-dimethylbutanoyl)pyrrolidine-2-carboxamide (200 mg, 424 umol) in DCM (4 mL). Then added DMP (270 mg, 636 umol) was added and the mixture was stirred at 25° C. for 3 hrs. On completion, the mixture was diluted with Na₂S₂O₃ and adjusted to pH 7-8 with a solution of Na₂CO₃. The mixture was then extracted with DCM (30 mL×3). The combined organic layers were dried over Na₂SO₄, filtered and concentrated under reduced pressure to give the title compound (90 mg) as a yellow solid. LC-MS (ESI+) m/z 470.3 (M+H)⁺.

### (S)-6-(5-omopyrazin-2-yl)-3-(2-(methoxymethoxy)phenyl)-5-methyl-6,7,8,9-tetrahydro-5H-pyrido[3′,4′:4,5]pyrrolo[2,3-c]pyridazine (Intermediate OR) & (R)-6-(5-omopyrazin-2-yl)-3-(2-(methoxymethoxy)phenyl)-5-methyl-6,7,8,9-tetrahydro-5H-pyrido[3′,4′:4,5]pyrrolo[2,3-c]pyridazine (Intermediate OS)

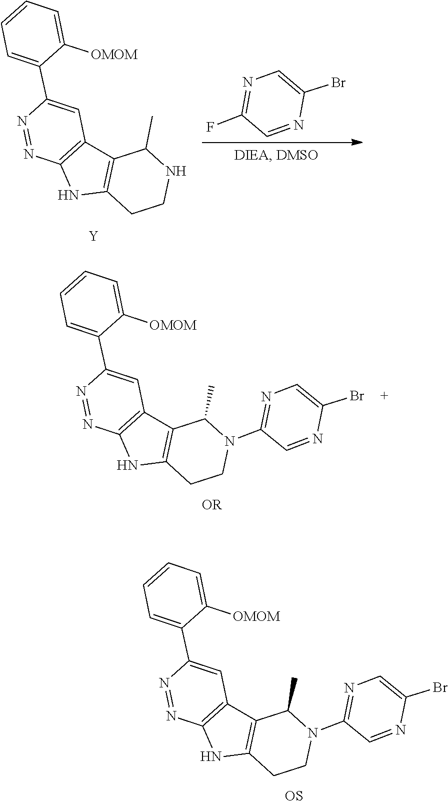

To a solution of 3-(2-(methoxymethoxy)phenyl)-5-methyl-6,7,8,9-tetrahydro-5H-pyrido[3′,4′:4,5] pyrrolo[2,3-c]pyridazine (5 g, 15 mmol, Intermediate Y) in DMSO (50 mL) was added DIEA (5.98 g, 46.24 mmol), and 2-omo-5-fluoropyrazine(4.09 g, 23.1 mmol). The mixture was stirred at 60° C. for 2 hrs. On completion, the reaction mixture was concentrated in vacuo to give a residue. The residue was purified by column chromatography (SiO₂, Petroleum ether/Ethyl acetate=3/1 to 0/1) then separated by SFC (column: DAICEL CHIRALCEL OJ(250 mm*50 mm,10 um);mobile phase: [0.1% NH3H2O ETOH];B %: 60%-60%,4.5; 150 min) to give (S)-6-(5-omopyrazin-2-yl)-3-(2-(methoxymethoxy)phenyl)-5-methyl-6,7,8,9-tetrahydro-5H-pyrido[3′,4′:4,5]pyrrolo[2,3-c]pyridazine (2 g, 27% yield) and (R)-6-(5-omopyrazin-2-yl)-3-(2-(methoxymethoxy)phenyl)-5-methyl-6,7,8,9-tetrahydro-5H-pyrido[3′,4′:4,5]pyrrolo[2,3-c]pyridazine (2.3 g, 30% yield) as a yellow solid. LC-MS (ESI⁺) m/z 480.9 (M+H)⁺ and 481.2 (M+H)⁺.

### (R)-2-(5-methyl-6-(5-(piperidin-4-yl)pyrazin-2-yl)-6,7,8,9-tetrahydro-5H-pyrido[3′,4′:4,5]pyrrolo[2,3-c]pyridazin-3-yl)phenol (Intermediate OT)

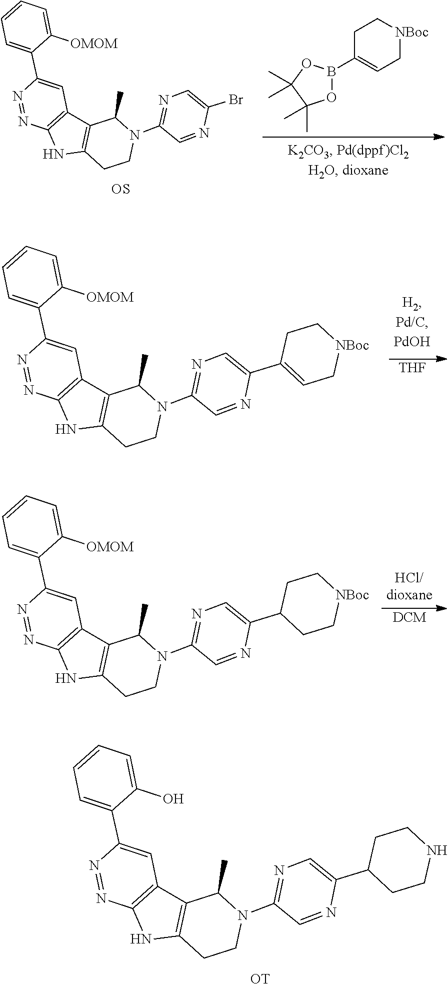

Step 1-(R)-tert-butyl 4-(5-(3-(2-(methoxymethoxy)phenyl)-5-methyl-7,8-dihydro-5H-pyrido [3′,4′:4,5]pyrrolo[2,3-c]pyridazin-6(9H)-yl)pyrazin-2-yl)-5,6-dihydropyridine-1(2H)-carboxylate. To a solution of (R)-6-(5-omopyrazin-2-yl)-3-(2-(methoxymethoxy)phenyl)-5-methyl-6,7,8,9-tetrahydro-5H-pyrido[3′,4′:4,5]pyrrolo[2,3-c]pyridazine (2.00 g, 4.16 mmol, Intermediate OS) in dioxane (20 mL) and H₂O (5 mL) was added K₂CO₃ (1.72 g, 12.5 mmol), Pd(dppf)C12 (304 mg, 416 umol) and tert-butyl 4-(4,4,5,5-tetramethyl-1,3,2-dioxaborolan-2-yl)-5,6-dihydropyridine-1(2H)-carboxylate (1.93 g, 6.23 mmol, CAS #286961-14-6). The reaction was then stirred at 80° C. for 12 hrs. On completion, the reaction mixture was concentrated in vacuo to give a residue. The residue was purified by column chromatography (SiO₂, Petroleum ether/Ethyl acetate=3/1 to 0/1) to give the title compound (1.3 g, 50% yield) as a brown solid. LC-MS (ESI⁺) m/z 584.7 (M+H)⁺.

Step 2-(R)-tert-butyl 4-(5-(3-(2-(methoxymethoxy)phenyl)-5-methyl-7,8-dihydro-5H-pyrido[3′,4′:4,5]pyrrolo[2,3-c]pyridazin-6(9H)-yl)pyrazin-2-yl)piperidine-1-carboxylate. To a solution of (R)-tert-butyl 4-(5-(3-(2-(methoxymethoxy)phenyl)-5-methyl-7,8-dihydro-5H-pyrido[3′,4′:4,5]pyrrolo[2,3-c]pyridazin-6(9H)-yl)pyrazin-2-yl)-5,6-dihydropyridine-1(2H)-carboxylate (1.3 g, 2.23 mmol) in THF (15 mL) was added Pd/C (650 mg, 10 wt %), Pd(OH)₂ (650 mg, 20 wt %) and H₂ (15 PSI). The reaction mixture was stirred at 25° C. for 12 hrs. On completion, the reaction mixture was filtered to give the title compound (1.1 g) as a brown solid. LC-MS (ESI⁺) m/z 586.6 (M+H)⁺.

Step 3-(R)-2-(5-methyl-6-(5-(piperidin-4-yl)pyrazin-2-yl)-6,7,8,9-tetrahydro-5H-pyrido[3′,4′:4,5]pyrrolo[2,3-c]pyridazin-3-yl)phenol. To a solution of (R)-tert-butyl 4-(5-(3-(2-(methoxymethoxy)phenyl)-5-methyl-7,8-dihydro-5H-pyrido[3′,4′:4,5]pyrrolo[2,3-c]pyridazin-6(9H)-yl)pyrazin-2-yl)piperidine-1-carboxylate (1.1 g, 1.9 mmol) in DCM (10 mL) was added HCl/dioxane (8 M, 5 mL). The reaction mixture was then stirred at 25° C. for 1 hour. On completion, the reaction mixture was concentrated in vacuo to give the title compound (1 g, HCl) as a green solid. LC-MS (ESI⁺) m/z 442.2 (M+H)⁺.

### (R)-2-(6-(5-(1-(2-azaspiro[3.3]heptan-6-yl)piperidin-4-yl)pyrazin-2-yl)-5-methyl-6,7,8,9-tetrahydro-5H-pyrido[3′,4′:4,5]pyrrolo[2,3-c]pyridazin-3-yl)phenol (Intermediate OU)

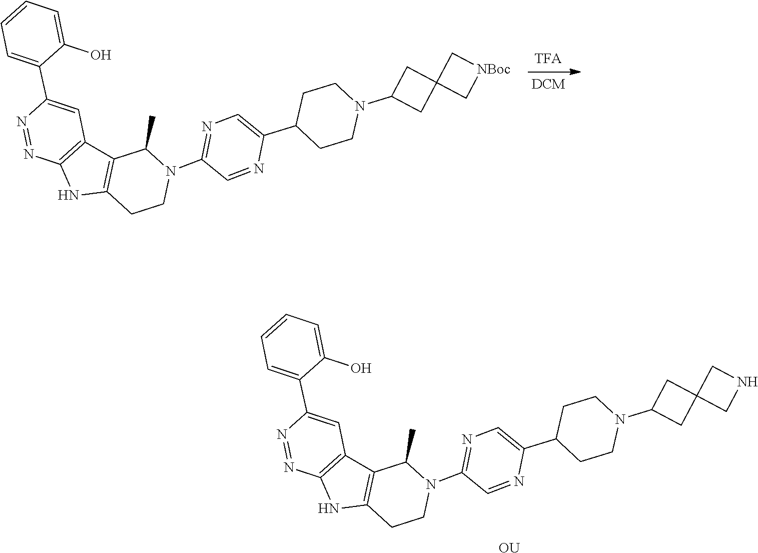 [SMILES: C[C@@H]1c2c([nH]c3nnc(-c4ccccc4O)cc23)CCN1c1cnc(C2CCN(C3CC4(C3)CN(C(=O)OC(C)(C)C)C4)CC2)cn1 ; C[C@@H]1c2c([nH]c3nnc(-c4ccccc4O)cc23)CCN1c1cnc(C2CCN(C3CC4(CNC4)C3)CC2)cn1 ; "OU"]

Step 1-(R)-tert-butyl 6-(4-(5-(3-(2-hydroxyphenyl)-5-methyl-7,8-dihydro-5H-pyrido[3′,4′:4,5] pyrrolo[2,3-c]pyridazin-6(9H)-yl)pyrazin-2-yl)piperidin-1-yl)-2-azaspiro[3.3]heptane-2-carboxylate. To a solution of (R)-2-(5-methyl-6-(5-(piperidin-4-yl)pyrazin-2-yl)-6,7,8,9-tetrahydro-5H-pyrido[3′,4′:4,5]pyrrolo[2,3-c]pyridazin-3-yl)phenol (500 mg, 1.13 mmol, Intermediate OT) in THF (3 mL) and DMSO (3 mL) was added AcOK (333 mg, 3.40 mmol) at 25° C. and the mixture was stirred for 1 hr. Then AcOH (272 mg, 4.53 mmol) and tert-butyl 6-oxo-2-azaspiro[3.3]heptane-2-carboxylate (239 mg, 1.13 mmol) were added at 25° C. and the mixture was stirred for 1 hr. Finally, NaBH(OAc)₃ (720 mg, 3.40 mmol) was added at 0° C. then the reaction was stirred at 25° C. for 3 hrs. On completion, the reaction mixture was diluted with H₂O (30 mL) and extracted with EA (10 mL×3). The combined organic layers were washed with brine (20 mL×2), dried over Na₂SO₄, filtered and concentrated under reduced pressure to give the title compound (320 mg) as a yellow solid. LC-MS (ESI⁺) m/z 637.6 (M+H)⁺.

Step 2-(R)-2-(6-(5-(1-(2-azaspiro[3.3]heptan-6-yl)piperidin-4-yl)pyrazin-2-yl)-5-methyl-6,7,8,9-tetrahydro-5H-pyrido[3′,4′:4,5]pyrrolo[2,3-c]pyridazin-3-yl)phenol. To a solution of(R)-tert-butyl 6-(4-(5-(3-(2-hydroxyphenyl)-5-methyl-7,8-dihydro-5H-pyrido[3′,4′:4,5]pyrrolo[2,3-c]pyridazin-6(9H)-yl)pyrazin-2-yl)piperidin-1-yl)-2-azaspiro[3.3]heptane-2-carboxylate (300 mg, 471 umol) in DCM (0.5 mL) was added TFA (2.31 g, 20.3 mmol). The mixture was stirred at 25° C. for 0.5 hr. On completion, the reaction mixture was concentrated in vacuo to give a residue. The residue was purified by reversed-phase HPLC(0.1% TFA condition) to give the title compound (235 mg, 77% yield, TFA) as a yellow solid. LC-MS (ESI⁺) m/z 537.2 (M+H)⁺.

### 6-(5-Bromopyrimidin-2-yl)-3-(2-(methoxymethoxy)phenyl)-5-methyl-6,7,8,9-tetrahydro-5H-pyrido[3′,4′:4,5]pyrrolo[2,3-c]pyridazine (Intermediate OV)

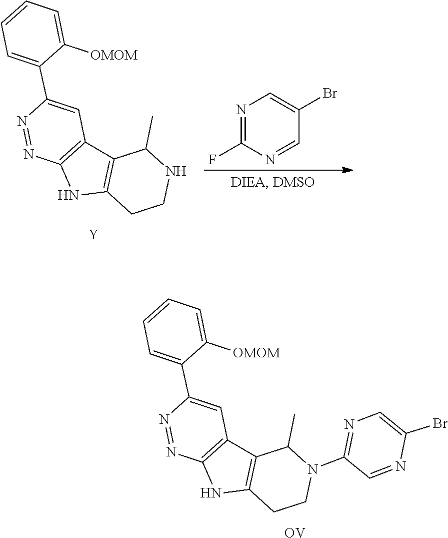

To a solution of 12-[2-(methoxymethoxy)phenyl]-3-methyl-4,8,10,11-tetrazatricyclo[7.4.0.0^{2,7}]trideca-1(9),2(7),10,12-tetraene (5 g, 15 mmol, Intermediate Y) in DMSO (50 mL) was added DIEA (7.97 g, 61.6 mmol). Then to the mixture was added 5-bromo-2-fluoro-pyrimidine (4.09 g, 23.1 mmol, CAS #62802-38-4) and the mixture was stirred at 100° C. for 2 hours. On completion, the reaction mixture was extracted with ethyl acetate (250 mL). The combined organic layers were washed with brine (250 mL), dried with anhydrous Na₂SO₄, filtered and concentrated under reduced pressure to give a residue. The residue was purified by silica gel chromatography (DCM/Ethyl acetate=10/1, 5/1) then re-purified by silica gel chromatography (DCM/methanol=10/1) to afford the title compound (5 g, 65% yield) as black solid. LC-MS (ESI⁺) m/z 481.2 (M+H)⁺. ¹H NMR (400 MHz, DMSO-d₆) δ ppm 1.49 (d, J=6.63 Hz, 4H) 3.35 (br s, 7H) 5.16-5.21 (m, 1H) 5.22-5.27 (m, 1H) 5.86 (q, J=6.46 Hz, 1H) 7.15 (td, J=7.44, 0.88 Hz, 1H) 7.22-7.30 (m, 1H) 7.35-7.44 (m, 1H) 7.71 (dd, J=7.63, 1.75 Hz, 1H) 8.07 (s, 1H) 8.51 (s, 2H) 12.24 (s, 1H).

### (R)-6-(5-bromopyrimidin-2-yl)-3-(2-(methoxymethoxy)phenyl)-5-methyl-6,7,8,9-tetrahydro-5H-pyrido[3′,4′:4,5]pyrrolo[2,3-c]pyridazine (Intermediate OW) and (S)-6-(5-bromopyrimidin-2-yl)-3-(2-(methoxymethoxy)phenyl)-5-methyl-6,7,8,9-tetrahydro-5H-pyrido[3′,4′:4,5]pyrrolo[2,3-c]pyridazine (Intermediate OX)

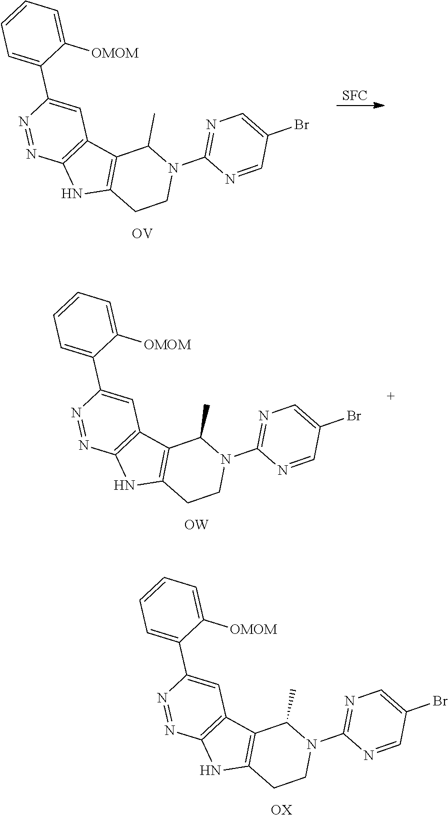

4-(5-bromopyrimidin-2-yl)-12-[2-(methoxymethoxy)phenyl]-3-methyl-4,8,10,11-tetrazatricyclo[7.4.0.0^{2,7}]trideca-1(9),2(7),10,12-tetraene (15 g, Intermediate OV) was separated by SFC (column: DAICEL CHIRALPAK AD (250 mm*30 mm, 10 um); mobile phase: [0.1% NH₃H₂O MEOH]; B %: 47%-47%, 6; 100 min) to give (R)-6-(5-bromopyrimidin-2-yl)-3-(2-(methoxymethoxy)phenyl)-5-methyl-6,7,8,9-tetrahydro-5H-pyrido[3′,4′:4,5]pyrrolo[2,3-c]pyridazine (6.7 g) and (S)-6-(5-bromopyrimidin-2-yl)-3-(2-(methoxymethoxy)phenyl)-5-methyl-6,7,8,9-tetrahydro-5H-pyrido[3′,4′:4,5]pyrrolo[2,3-c]pyridazine (7.2 g) as yellow solids. LC-MS (ESI⁺) m/z 480.8 (M+H)⁺.

### (R)-1-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-4-one (Intermediate OY)

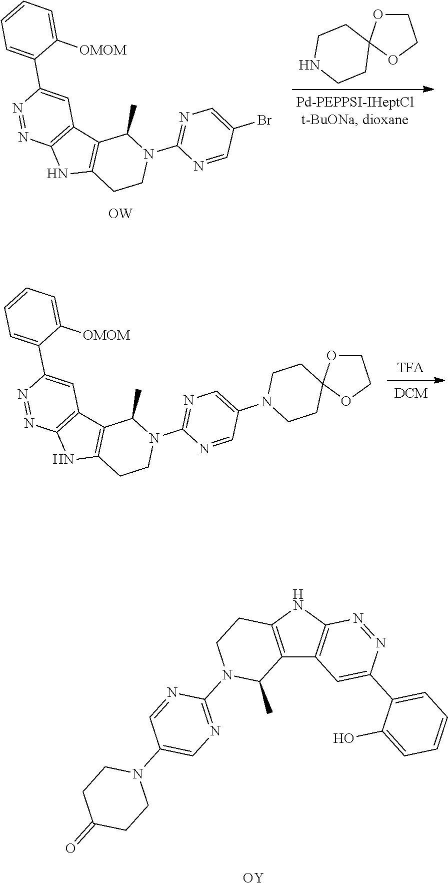

Step 1-(R)-8-(2-(3-(2-(methoxymethoxy)phenyl)-5-methyl-7,8-dihydro-5H-pyrido[3′,4′:4,5]pyrrolo[2,3-c]pyridazin-6(9H)-yl)pyrimidin-5-yl)-1,4-dioxa-8-azaspiro[4.5]decane. A mixture of (3R)-4-(5-bromopyrimidin-2-yl)-12-[2-(methoxymethoxy)phenyl]-3-methyl-4,8,10,11-tetrazatricyclo[7.4.0.0^{2,7}]trideca-1(9),2(7),10,12-tetraene (2.30 g, 4.78 mmol, Intermediate OW), 1,4-dioxa-8-azaspiro[4.5]decane (2.05 g, 14.3 mol, 1.83 mL, CAS #177-11-7), 1,3-bis[2,6-bis(1-propylbutyl)phenyl]-4,5-dichloro-2H-imidazol-1-ium-2-ide;3-chloropyridine dichloropalladium (465 mg, 478 umol), and tBuONa (2 M, 7.17 mL) in dioxane (300 mL) was degassed and purged with N₂ three times. Then the mixture was stirred at 110° C. for 12 hrs under N₂ atmosphere. On completion, the reaction mixture was concentrated under reduced pressure to remove dioxane. The residue was dissolved with DCM (30 mL) and the suspension was added silica gel and dried in vacuo to give a residue. The residue was purified by column chromatography (SiO₂, Petroleum ether/methyl alcohol=40/1) to give the title compound (2.4 g, 66% yield) as a yellow solid. LC-MS (ESI⁺) m/z 544.4 (M+H)⁺. ¹H NMR (400 MHz, DMSO-d₆) δ ppm 1.52 (br d, J=5.38 Hz, 4H) 1.72 (br s, 3H) 2.73 (br s, 2H) 3.08 (br s, 2H) 3.31 (br s, 3H) 3.84 (s, 4H) 3.88-3.92 (m, 1H) 3.89 (s, 2H) 4.91-5.02 (m, 1H) 5.17-5.21 (m, 1H) 5.22-5.27 (m, 1H) 5.75 (s, 1H) 7.15 (br t, J=7.32 Hz, 1H) 7.26 (br d, J=8.13 Hz, 1H) 7.37-7.42 (m, 1H) 7.71 (br d, J=7.25 Hz, 1H) 7.97-8.09 (m, 1H) 8.25 (s, 2H).

Step 2-(R)-1-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-4-one. To a solution of 8-[2-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]pyrimidin-5-yl]-1,4-dioxa-8-azaspiro[4.5]decane (1.2 g, 2.2 mmol) in DCM (12 mL) was added TFA (6 mL). The mixture was stirred at 25° C. for 12 hrs. On completion, the reaction mixture was extracted with NaHCO₃ (30 mL). The combined organic layers were dried over anhydrous Na₂SO₄, filtered and concentrated under reduced pressure to give the title compound (900 mg, 29% yield, TFA) as a yellow solid. LC-MS (ESI⁺) m/z 456.2 (M+H)⁺.

### (R)-2-(6-(5-(4-(2,6-Diazaspiro[3.3]heptan-2-yl)piperidin-1-yl)pyrimidin-2-yl)-5-methyl-6,7,8,9-tetrahydro-5H-pyrido[3′,4′:4,5]pyrrolo[2,3-c]pyridazin-3-yl)phenol (Intermediate OZ)

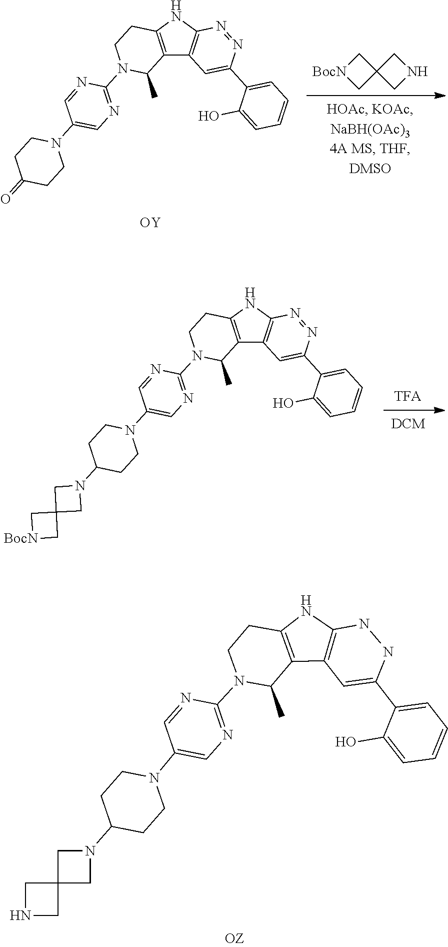

Step 1-(R)-tert-butyl 6-(1-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-4-yl)-2,6-diazaspiro[3.3]heptane-2-carboxylate. To a solution of tert-butyl 2,6-diazaspiro[3.3]heptane-2-carboxylate (384.4 mg, 1.58 mmol) in DMSO (2 mL) and THF (20 mL) was added KOAc (310 mg, 3.16 mmol) and the mixture was stirred at 25° C. for 0.5 hrs. Then 1-[2-[(3R)-12-(2-hydroxyphenyl)-3-methyl-4,8,10,11-tetrazatricyclo[7.4.0.02,7]trideca-1(9),2(7),10,12-tetraen-4-yl]pyrimidin-5-yl]piperidin-4-one (900 mg, 1.58 mmol, TFA), HOAc (285 mg, 4.74 mmol, Intermediate OY) and 4 Å molecular sieves (50 mg, 1.58 mmol) were added and the mixture was stirred at 25° C. for 0.5 hrs. Finally, to the mixture was added NaBH(OAc)₃ (1.00 g, 4.74 mmol) at 0° C. and the mixture was stirred at 0-25° C. for 13 hrs. On completion, the reaction mixture was quenched with H₂O (0.5 mL). Then the crude product was purified by reversed-phase HPLC (0.1% FA condition) and lyophilized to give the title compound as a yellow solid (320 mg, 28 yield). LC-MS (ESI⁺) m/z 638.5 (M+H)⁺.

Step 2-(R)-2-(6-(5-(4-(2,6-diazaspiro[3.3]heptan-2-yl)piperidin-1-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 6-[1-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-4-piperidyl]-2,6-diazaspiro[3.3]heptane-2-carboxylate (270 mg, 420 umol) in DCM (5 mL) was added TFA (1.66 g, 14.5 mmol). The mixture was stirred at 25° C. for 0.5 hrs. On completion, the reaction mixture was concentrated under reduced pressure to remove DCM and give a residue. The crude product was purified by reversed-phase HPLC (0.1% FA condition) and lyophilized to give the title compound as a yellow solid (226 mg, 69% yield, FA). LC-MS (ESI⁺) m/z 538.4 (M+H)⁺.

### (2S,4R)-1-((R)-2-Amino-5-hydroxy-3,3-dimethylpentanoyl)-N-(4-ethynylbenzyl)-4-hydroxypyrrolidine-2-carboxamide (Intermediate PA)

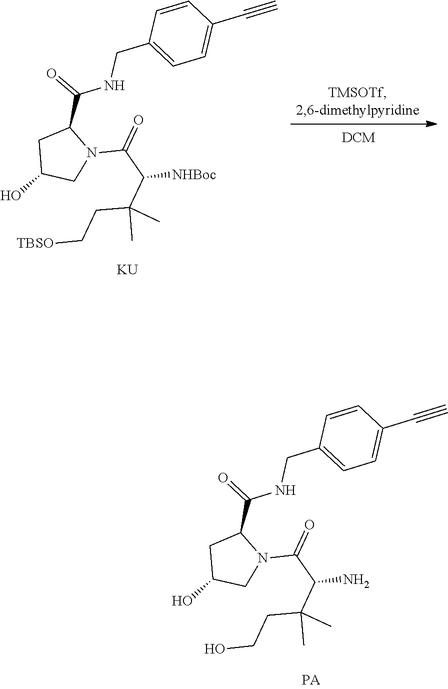

To a solution of tert-butyl ((R)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl)carbamate (100 mg, 166 umol, Intermediate KU) in DCM (0.5 mL) was added TMSOTf (111 mg, 498 umol, 90.0 uL) and 2,6-dimethylpyridine (89.9 mg, 831 umol, 97.7 uL). The mixture was stirred at 25° C. for 2 hrs. The mixture was concentrated under reduce pressure to give a residue. The crude product was purified by reversed phase Flash (0.1% FA) to give the title compound (60 mg, FA Salt) as white solid. LC-MS (ESI⁺) m/z 502.3 (M+114)⁺.

### (S)-2-(8-(2-(Methylamino)ethyl)-6,6a,7,8,9,10-hexahydro-5H-pyrazino[1′,2′:4,5]pyrazino[2,3-c]pyridazin-2-yl)phenol (Intermediate PB)

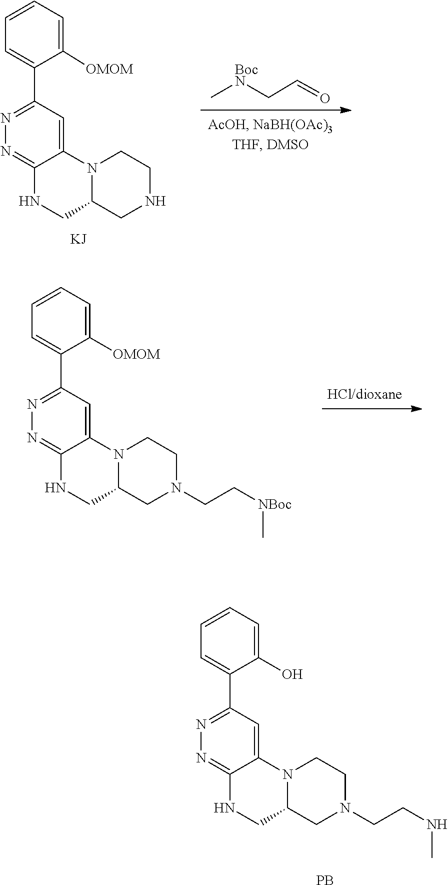

Step 1-(S)-tert-butyl (2-(2-(2-(methoxymethoxy)phenyl)-6a,7,9,10-tetrahydro-5H-pyrazino[1′,2′:4,5]pyrazino[2,3-c]pyridazin-8(6H)-yl)ethyl)(methyl)carbamate. To a solution of (10R)-4-[2-(methoxymethoxy)phenyl]-1,5,6,8,12-pentazatricyclo[8.4.0.02,7]tetradeca-2,4,6-triene (1 g, 3 mmol, Intermediate KJ) in THF (15 mL), and DMSO (3 mL) was added AcOH (550 mg, 9.16 mmol, 524 uL), tert-butyl N-methyl-N-(2-oxoethyl)carbamate (635 mg, 3.67 mmol, CAS #123387-72-4) at 25° C. for 1 hr. Next, NaBH(OAc)₃ (1.94 g, 9.16 mmol) was added at 0° C. and the mixture was stirred at 25° C. for 3 hrs. On completion, the reaction mixture was quenched with H₂O (3 mL) at 25° C., and then diluted with H₂O (45 mL) and extracted with EA (15 mL×3). The combined organic layers were washed with aqueous NaCl (15 mL×3), dried over Na₂SO₄, filtered and concentrated under reduced pressure to give a residue. The residue was purified by column chromatography (SiO₂, Petroleum DCM: MeOH=60/1 to 10/1) to give the title compound (1.10 g, 72% yield) as a yellow solid. LC-MS (ESI⁺) m/z 485.4 (M+H)⁺.

Step 2-(S)-2-(8-(2-(methylamino)ethyl)-6,6a,7,8,9,10-hexahydro-5H-pyrazino[1′,2′:4,5]pyrazino[2,3-c]pyridazin-2-yl)phenol. A solution of tert-butyl N-[2-[(10S)-4-[2-(methoxymethoxy)phenyl]-1,5,6,8,12-pentazatricyclo[8.4.0.02,7]tetradeca-2,4,6-trien-12-yl]ethyl]-N-methyl-carbamate (1 g, 2 mmol) in HCl/dioxane (10 mL) was stirred at 25° C. for 2 hrs. On completion, the reaction was concentrated under reduced pressure to give a residue. The residue was purified by prep-HPLC (HCl) to give the title compound (630 mg, 72% yield) as a yellow solid. LC-MS (ESI⁺) m/z 341.1 (M+H)⁺.

### (S)-2-(8-(2-(Methyl(piperidin-4-yl)amino)ethyl)-6,6a,7,8,9,10-hexahydro-5H-pyrazino[1′,2′:4,5]pyrazino[2,3-c]pyridazin-2-yl)phenol (Intermediate PC)

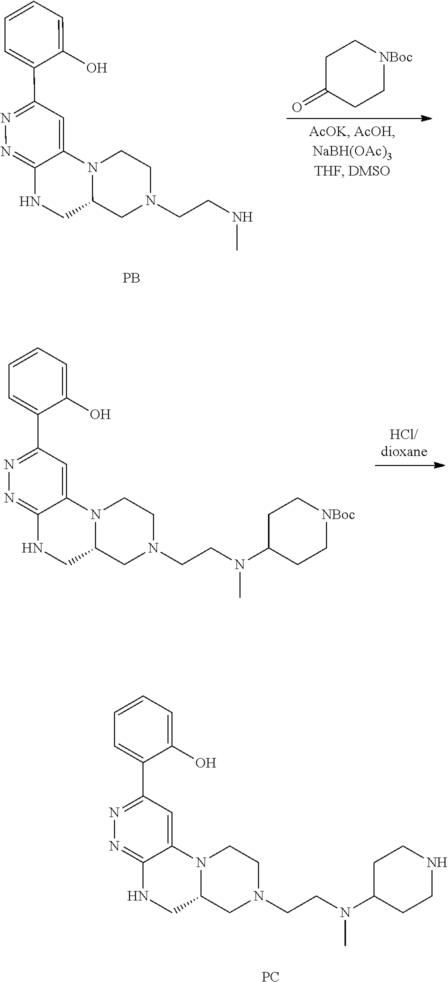 [SMILES: CNCCN1CCN2c3cc(-c4ccccc4O)nnc3NC[C@H]2C1 ; CC(C)(C)OC(=O)N1CCC(=O)CC1 ; "PB" ; CN(CCN1CCN2c3cc(-c4ccccc4O)nnc3NC[C@H]2C1)C1CCN(C(=O)OC(C)(C)C)CC1 ; CN(CCN1CCN2c3cc(-c4ccccc4O)nnc3NC[C@H]2C1)C1CCNCC1 ; "PC"]

Step 1-(S)-tert-butyl 4-((2-(2-(2-hydroxyphenyl)-6a,7,9,10-tetrahydro-5H-pyrazino[1′,2′:4,5]pyrazino[2,3-c]pyridazin-8(6H)-yl)ethyl)(methyl)amino)piperidine-1-carboxylate. To a solution of 2-[(10S)-12-[2-(methylamino)ethyl]-1,5,6,8,12-pentazatricyclo[8.4.0.02,7]tetradeca-2,4,6-trien-4-yl]phenol (580 mg, 1.70 mmol, Intermediate PB) in THF (10 mL), DMSO (2 mL) was added AcOK (836 mg, 8.52 mmol) at 25° C. for 0.5 hrs. Then AcOH (307 mg, 5.11 mmol, 292 uL), and tert-butyl 4-oxopiperidine-1-carboxylate (1.02 g, 5.11 mmol) were added at rt and stirred for 1 hr. At last, to the solution was added NaBH(OAc)₃ (1.08 g, 5.11 mmol) at 0° C. The mixture was then stirred at 25° C. for 2 hrs. On completion, the reaction was added water (2 ml), and then concentrated under reduced pressure to give the residue. The residue was purified by prep-HPLC (HCl) to give the title compound (580 mg, 60% yield) as a yellow solid. LC-MS (ESI⁺) m/z 524.3 (M+H)⁺.

Step 2-(S)-2-(8-(2-(methyl(piperidin-4-yl)amino)ethyl)-6,6a,7,8,9,10-hexahydro-5H-pyrazino[1′,2′:4,5]pyrazino[2,3-c]pyridazin-2-yl)phenol. A solution of tert-butyl 4-[2-[(10S)-4-(2-hydroxyphenyl)-1,5,6,8,12-pentazatricyclo[8.4.0.02,7]tetradeca-2,4,6-trien-12-yl]ethyl-methylamino]piperidine-1-carboxylate (100 mg, 191 umol) in HCl/dioxane (0.5 mL) and DCM (1 mL) was stirred at 25° C. for 2 hrs. On completion, the reaction was concentrated under reduced pressure to give the title compound (100 mg, HCl) as a yellow solid. LC-MS (ESI⁺) m/z 424.1 (M+H)⁺.

### Tert-butyl 4-(2-fluoropyrimidin-5-yl)-[1, 3′-bipiperidine]-1′-carboxylate (Intermediate PD)

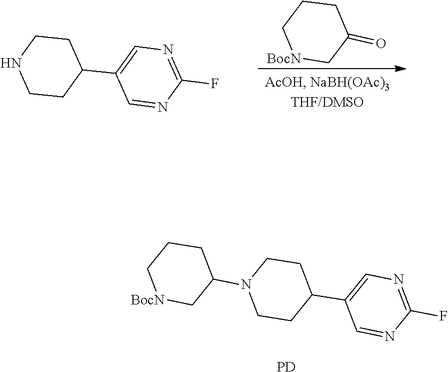 [SMILES: Fc1ncc(C2CCNCC2)cn1 ; CC(C)(C)OC(=O)N1CCCC(=O)C1 ; CC(C)(C)OC(=O)N1CCCC(N2CCC(c3cnc(F)nc3)CC2)C1 ; "PD"]

To a solution of 2-fluoro-5-(4-piperidyl)pyrimidine (1.5 g, 8.3 mmol, synthesized via Step 1 of Intermediate IR) in DMSO (2 mL) and THF (1 mL) was added HOAc (1.49 g, 24.8 mmol, 1.42 mL) and tert-butyl 3-oxopiperidine-1-carboxylate (4.12 g, 20.7 mmol) at 25° C. and the mixture was stirred for 2 hrs. Then NaBH(OAc) 3 (4.39 g, 20.7 mmol) was added at 0° C. and the mixture was stirred at 0-25° C. for 12 hrs. On completion, the residue was diluted with water (60 mL) and extracted with ethyl acetate (40 mL×3). The combined organic layers were washed with brine (50 mL), dried over Na₂SO₄, filtered and concentrated under reduced pressure to give the title compound (900 g, 27% yield) as a yellow solid. LC-MS (ESI⁺) m/z 365.5 (M+H)⁺.

### 2-((6aS)-8-(5-([1,3′-bipiperidin]-4-yl)pyrimidin-2-yl)-6,6a,7,8,9,10-hexahydro-5H-pyrazino[1′,2′:4,5]pyrazino[2,3-c]pyridazin-2-yl)phenol (Intermediate PE)

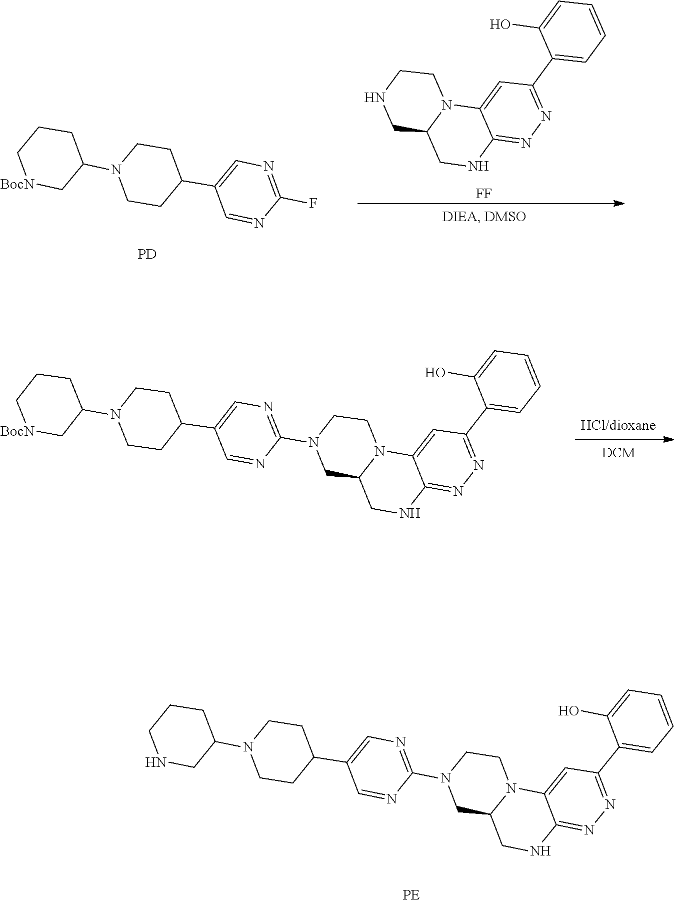

Step 1-Tert-butyl 4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)-[1,3′-bipiperidine]-1′-carboxylate. To a solution of 2-[(1OR)-1,5,6,8,12-pentazatricyclo[8.4.0.02,7]tetradeca-2,4,6-trien-4-yl]phenol (300 mg, 1.06 mmol, Intermediate FF) in DMSO (10 mL) was added tert-butyl 3-[4-(2-fluoropyrimidin-5-yl)-1-piperidyl]piperidine-1-carboxylate (502 mg, 1.38 mmol, Intermediate PD) and DIEA (411 mg, 3.18 mmol, 553 uL). Then the mixture was stirred at 80° C. for 12 hrs. On completion, the mixture was diluted with DMSO (2 ml), and the residue was purified by prep-HPLC (HCl) to give the title compound (350 mg, 46% yield) as a yellow solid. LC-MS (ESI⁺) m/z 628.5 (M+H)⁺.

Step 2-2-((6aS)-8-(5-([1,3′-bipiperidin]-4-yl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl 3-[4-[2-[(10S)-4-(2-hydroxyphenyl)-1, 5, 6, 8, 12-pentazatricyclo [8.4.0.02, 7] tetradeca-2, 4, 6-trien-12-yl] pyrimidin-5-yl]-1-piperidyl] piperidine-1-carboxylate (350 mg, 558 umol) in DCM (5 mL) was added HCl/dioxane (4 M, 1 mL) then the mixture was stirred at 25° C. for 2 hrs. On completion, the reaction mixture was concentrated under reduced pressure to remove DCM and HCl/dioxane to give the title compound (300 mg, HCl) as a brown solid. LC-MS (ESI⁺) m/z 528.4 (M+H)⁺.

### (S)-4-(4-(6-(2-(2-Hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)butanoic acid (Intermediate PF)

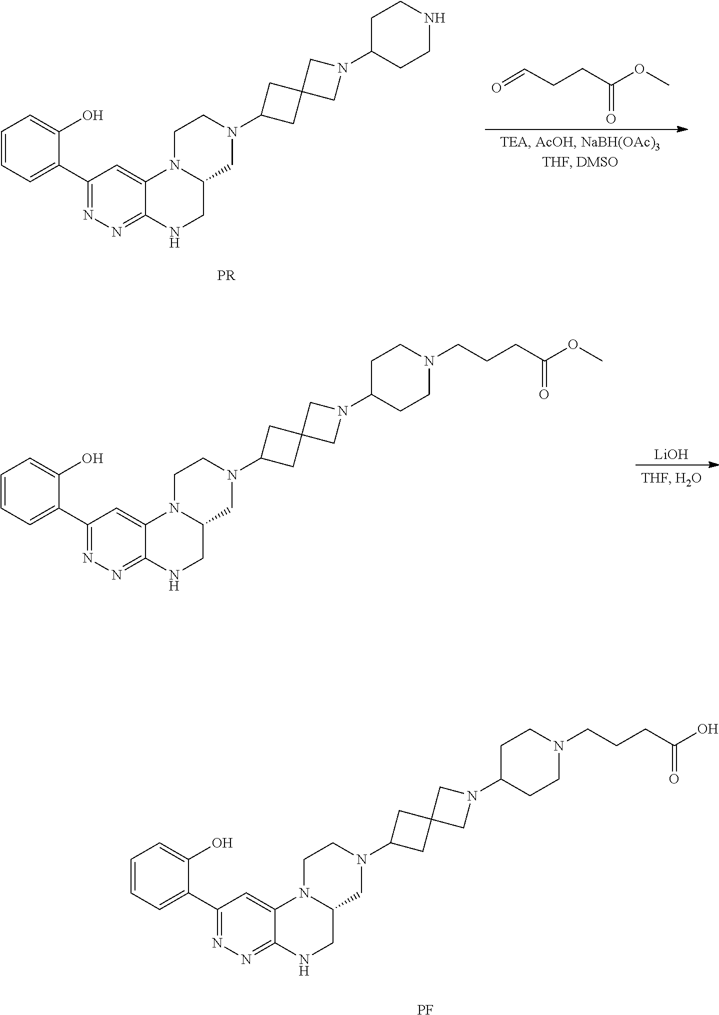

Step 1-(S)-methyl 4-(4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)butanoate. To a mixture of (S)-2-(8-(2-(piperidin-4-yl)-2-azaspiro[3.3]heptan-6-yl)-6,6a,7,8,9,10-hexahydro-5H-pyrazino[1′,2′:4,5]pyrazino[2,3-c]pyridazin-2-yl)phenol (200 mg, 348 umol, Intermediate PR) in THF (4 mL) and DMSO (0.8 mL) was added TEA (105 mg, 1.04 mmol). The mixture was stirred at 25° C. for 1 hr. Then methyl 4-oxobutanoate (40.3 mg, 348 umol, CAS #13865-19-5) and AcOH (62.6 mg, 1.04 mmol) was added and the mixture was stirred at 25° C. for 1 hr. Next. NaBH(OAc)₃ (184 mg, 869 umol) was added at 0° then the mixture was stirred at 25° C. for 1 hr. On completion, the reaction mixture was filtered and concentrated under reduced pressure to give a residue. The residue was purified by prep-HPLC (TFA condition) to give the title compound (203 mg, 72% yield) as a yellow solid. LC-MS (ESI+) m/z 562.2 (M+H)⁺.

Step 2-(S)-4-(4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)butanoic acid. To a mixture of (S)-methyl 4-(4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)butanoate (200 mg, 400 umol) in H₂O (10 mL) and THF (10 mL) was added LiOH—H₂O (59.8 mg, 1.42 mmol) at rt and the mixture was stirred for 1 hr. On completion, the reaction mixture was filtered and concentrated under reduced pressure to give a residue. The residue was purified by prep-HPLC (TFA condition) to give the title compound (93 mg, 38% yield) as a yellow solid. LC-MS (ESI+) m/z 548.4 (M+H)⁺.

### (2S,4R)-1-((S)-3,3-dimethyl-2-(8-oxooctanamido)butanoyl)-N-(4-ethynylbenzyl)-4-hydroxypyrrolidine-2-carboxamide (Intermediate PG)

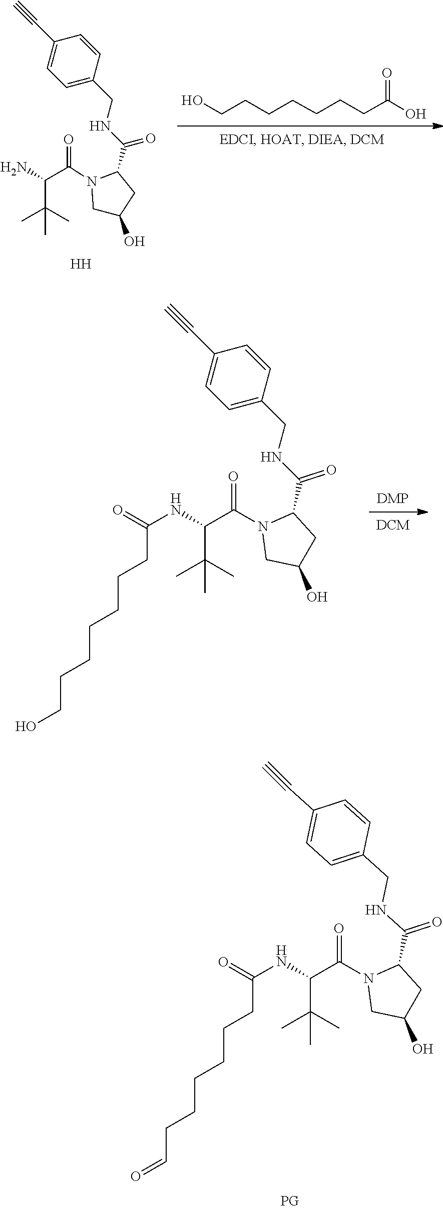

Step 1-(2S,4R)—N-(4-ethynylbenzyl)-4-hydroxy-1-((S)-2-(8-hydroxyoctanamido)-3,3-dimethylbutanoyl)pyrrolidine-2-carboxamide. To a mixture of (2S,4R)-1-((S)-2-amino-3,3-dimethylbutanoyl)-N-(4-ethynylbenzyl)-4-hydroxypyrrolidine-2-carboxamide (500 mg, 1.4 mmol, Intermediate HH), 8-hydroxyoctanoic acid (224 mg, 1.4 mmol, CAS #764-89-6) in DCM (10 mL) was added DIEA (723 mg, 5.6 mmol), HOAt (228 mg, 1.68 mmol) and EDCI (322 mg, 1.68 mmol). The mixture was then stirred at 25° C. for 2 hrs. On completion, the reaction mixture was filtered and concentrated under reduced pressure to give a residue. The residue was purified by prep-HPLC (FA condition) to give the title compound (521 mg, 67% yield) as a white solid. LC-MS (ESI+) m/z 500.4 (M+H)⁺.

Step 2-(2S,4R)-1-((S)-3,3-dimethyl-2-(8-oxooctanamido)butanoyl)-N-(4-ethynylbenzyl)-4-hydroxypyrrolidine-2-carboxamide. To a mixture of (2S,4R)—N-(4-ethynylbenzyl)-4-hydroxy-1-((S)-2-(8-hydroxyoctanamido)-3,3-dimethylbutanoyl)pyrrolidine-2-carboxamide (200 mg, 400 umol) in DCM (4 mL) was added DMP (255 mg, 600 umol). Then the mixture was stirred at 25° C. for 3 hrs. On completion, the mixture was diluted with the Na₂S₂O₃ and adjusted the pH to 7-8 with Na₂CO₃ solution. Then the mixture was extracted with DCM (30 mL×3). The combined organic layers were dried over Na₂SO₄, filtered and concentrated under reduced pressure to give the title compound (96 mg) as a yellow solid. LC-MS (ESI+) m/z 498.2 (M+H)⁺.

### 2-(Aminomethyl)-5-ethynylphenol (Intermediate PH)

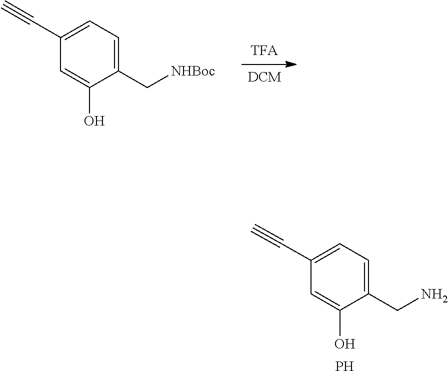

To a solution of tert-butyl N-[(4-ethynyl-2-hydroxy-phenyl)methyl]carbamate (1 g, 4.04 mmol, synthesized via Steps 1-3 of Intermediate HY) in DCM (10 mL) was added TFA (7.70 g, 67.53 mmol, 5 mL) 4 Å molecular sieves (2 g, 4 mmol). The mixture was stirred at 25° C. for 1 hr. On completion, the reaction mixture was filtered and concentrated under reduced pressure to give a residue. The crude product was purified by reversed-phase HPLC(0.1% FA condition) to give the title compound (250 mg, 32% yield, FA) as a white solid. LC-MS (ESI⁺) m/z 131.2 (M−NH₂)⁺

### (2S,4R)-1-((S)-2-amino-3,3-dimethylbutanoyl)-N-(4-ethynyl-2-hydroxybenzyl)-4-hydroxypyrrolidine-2-carboxamide (Intermediate PI)

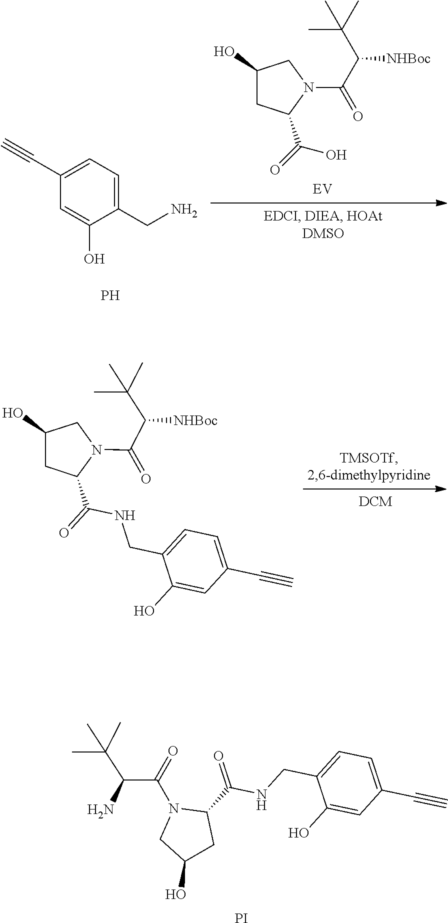

Step 1-Tert-butyl ((S)-1-((2S,4R)-2-((4-ethynyl-2-hydroxybenzyl)carbamoyl)-4-hydroxypyrrolidin-1-yl)-3,3-dimethyl-1-oxobutan-2-yl)carbamate. To a solution of (2S,4R)-1-[(2S)-2-(tert-butoxycarbonylamino)-3,3-dimethyl-butanoyl]-4-hydroxy-pyrrolidine-2-carboxylic acid (468.02 mg, 1.36 mmol, Intermediate EV) in DMSO (7 mL) was added EDCI (573.12 mg, 2.99 mmol), HOAt (406.92 mg, 2.99 mmol, 418.22 uL) and DIEA (878.15 mg, 6.79 mmol, 1.18 mL). After 10 mins of stirring at rt, added 2-(aminomethyl)-5-ethynyl-phenol (200 mg, 1.36 mmol, Intermediate PH) was added then the mixture was stirred at 25° C. for 12 hr. On completion, the reaction mixture was partitioned between EA (40 mL) and H₂O (20 mL). The organic phase was separated, washed with NaCl (10 mL×3), dried over Na₂SO₄, filtered and concentrated under reduced pressure to give a residue. The crude product was purified by reversed-phase HPLC(0.1% FA condition). Then the product was repurified by reversed-phase HPLC(NH3•H2O condition) to give the title compound (290 mg, 45% yield) as a white solid. LC-MS (ESI⁺) m/z 474.2(M+H)⁺.

Step 2-(2S,4R)-1-((S)-2-amino-3,3-dimethylbutanoyl)-N-(4-ethynyl-2-hydroxybenzyl)-4-hydroxypyrrolidine-2-carboxamide. To a solution of tert-butyl N-[(1S)-1-[(2S,4R)-2-[(4-ethynyl-2-hydroxy-phenyl)methylcarbamoyl]-4-hydroxy-pyrrolidine-1-carbonyl]-2,2-dimethyl-propyl]carbamate (150 mg, 320 umol) in DCM (1.5 mL) was added TMSOTf (422.40 mg, 1.90 mmol, 343.42 uL) and 2,6-dimethylpyridine (342.56 mg, 3.17 mmol). The mixture was then stirred at 25° C. for 1 hr. On completion, the crude product was purified by reversed-phase HPLC(0.1% FA condition). Then the product was repurified by reversed-phase HPLC(0.1% NH3•H₂O) to give the title compound (85 mg, 71% yield) as a white solid. LC-MS (ESI+) m/z 374.1 (M+H)⁺.

### Tert-butyl 4-(2-chloroacetyl) piperazine-1-carboxylate (Intermediate PJ)

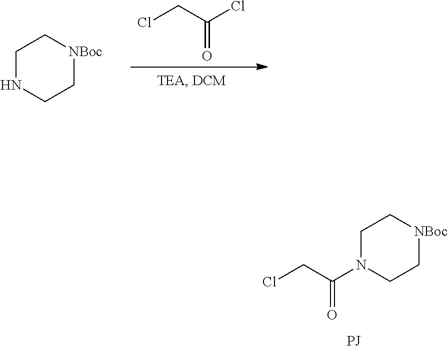 [SMILES: CC(C)(C)OC(=O)N1CCNCC1 ; O=C(Cl)CCl ; CC(C)(C)OC(=O)N1CCN(C(=O)CCl)CC1]

To a solution of tert-butyl piperazine-1-carboxylate (3 g, 16 mmol) in DCM (90 mL) was added TEA (1.63 g, 16.1 mmol, 2.24 mL) and was added 2-chloroacetyl chloride (2.00 g, 17.7 mmol, 1.41 mL) at 0° C. The mixture was stirred at 25° C. for 2 hrs. On completion, the reaction mixture was diluted with H₂O (30 mL) and extracted with DCM (40 mL×3). The combined organic layers were washed with aqueous NaCl (40 mL×3), dried over Na₂SO₄, filtered and concentrated under reduced pressure to give a residue. The residue was purified by column chromatography (SiO₂, Petroleum ether/Ethyl acetate=6/1 to 3/1) to give the title compound (3.47 g, 82% yield) as a yellow solid.

### (S)-2-(2-(2-hydroxyphenyl)-6a,7,9,10-tetrahydro-5H-pyrazino[1′,2′:4,5]pyrazino[2,3-c]pyridazin-8(6H)-yl)-1-(piperazin-1-yl)ethenone (Intermediate PK)

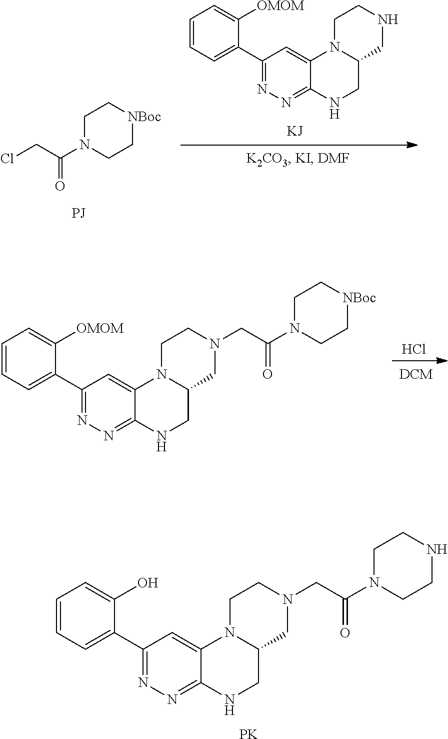

Step 1-(S)-tert-butyl 4-(2-(2-(2-(methoxymethoxy)phenyl)-6a,7,9,10-tetrahydro-5H-pyrazino[1′,2′:4,5]pyrazino[2,3-c]pyridazin-8(6H)-yl)acetyl)piperazine-1-carboxylate. To a solution of tert-butyl 4-(2-chloroacetyl)piperazine-1-carboxylate (1.32 g, 5.04 mmol, Intermediate PJ) in DMF (15 mL) was added K₂CO₃ (1.90 g, 13.8 mmol), KI (152 mg, 916 umol), and (10R)-4-[2-(methoxymethoxy)phenyl]-1,5,6,8,12-pentazatricyclo[8.4.0.02,7]tetradeca-2,4,6-triene (1.5 g, 4.58 mmol, Intermediate KJ). The mixture was then stirred at 25° C. for 4 hrs. On completion, the reaction was concentrated under reduced pressure to give the residue. The residue was purified by prep-HPLC (HCl) to give the title compound (1.9 g, 73% yield) as a yellow solid. LC-MS (ESI⁺) m/z 554.7 (M+H)⁺.

Step 2-(S)-2-(2-(2-hydroxyphenyl)-6a,7,9,10-tetrahydro-5H-pyrazino[1′,2′:4,5]pyrazino[2,3-c]pyridazin-8(6H)-yl)-1-(piperazin-1-yl)ethanone. To a solution of tert-butyl 4-[2-[(10S)-4-[2-(methoxymethoxy)phenyl]-1,5,6,8,12-pentazatricyclo[8.4.0.02,7]tetradeca-2,4,6-trien-12-yl]acetyl]piperazine-1-carboxylate (1.07 g, 1.93 mmol) in DCM (5 mL) was added HCl/dioxane (5 mL). The mixture was stirred at 25° C. for 2 hrs. On completion, to the reaction was added water (2 ml), and then concentrated under reduced pressure to give the residue. The residue was purified by prep-HPLC (HCl) to give the title compound (470 mg, 54% yield) as a yellow solid. LC-MS (ESI⁺) m/z 410.0 (M+H)⁺.

### (S)-Methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylate (Intermediate PL) and (S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylate (Intermediate PM)

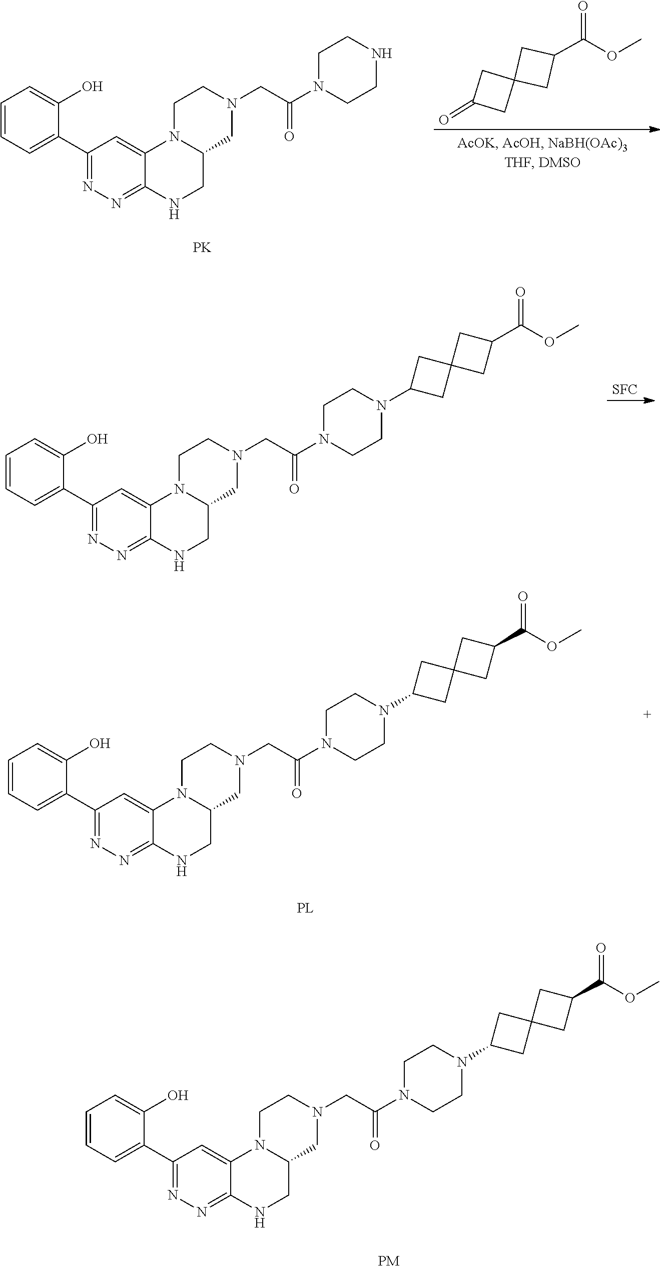

Step 1-(S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylate. To a solution of 2-[(10S)-4-(2-hydroxyphenyl)-1,5,6,8,12-pentazatricyclo[8.4.0.02,7]tetradeca-2,4,6-trien-12-yl]-1-piperazin-1-yl-ethanone (30 mg, 73.3 umol, Intermediate PK) in THF (1 mL), DMSO (0.2 mL) was added AcOK (36.0 mg, 366 umol) at 25° C. for 0.5 hrs. Next, AcOH (13.2 mg, 220 umol, 12.6 uL), and methyl 2-oxospiro [3.3] heptane-6-carboxylate (18.5 mg, 110 umol, CAS #1138480-98-4) was added to the mixture at rt and the mixture was stirred for 1 hr. Finally, to the solution was added NaBH(OAc)₃ (46.6 mg, 220 umol) and the mixture was stirred at 25° C. for 2 hrs. On completion, the reaction was concentrated under reduced pressure to give the residue. The residue was purified by prep-HPLC (HCl) to give the title compound (480 mg, 68% yield) as a white solid. LC-MS (ESI⁺) m/z 562.3 (M+H)⁺.

Step 2-(S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylate and (S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylate. (S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylate was purified by SFC (column: DAICEL CHIRALPAK IG (250 mm*30 mm, 10 um); mobile phase: [ACN/MeOH(0.1% NH3H2O)]; B %: 70%-70%,8.5; 90 min) to give the title compound as (S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylate 1 (210 mg, 43% yield) as a white solid (LC-MS (ESI⁺) m/z 562.4 (M+H)⁺) and (S)-methyl 6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylate (290 mg, 60% yield) as a white solid (LC-MS (ESI⁺) m/z 562.4 (M+H) ⁺).

### (S)-6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylic acid (Intermediate PN)

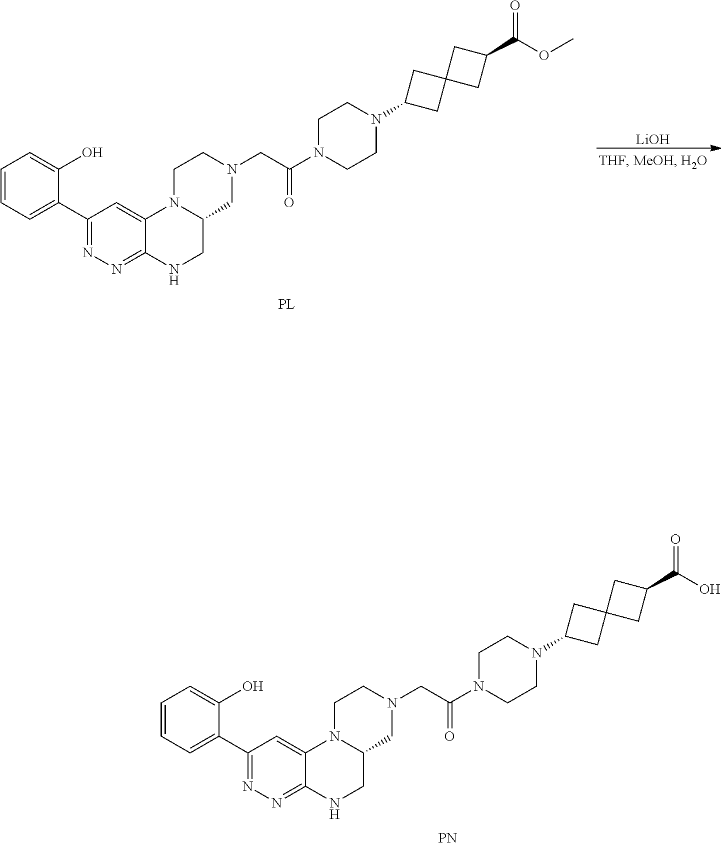 [SMILES: COC(=O)[C@H]1CC2(C1)C[C@H](N1CCN(C(=O)CN3CCN4c5cc(-c6ccccc6O)nnc5NC[C@H]4C3)CC1)C2 ; "LiOH" ; O=C(CN1CCN2c3cc(-c4ccccc4O)nnc3NC[C@H]2C1)N1CCN([C@H]2CC3(C[C@H](C(=O)O)C3)C2)CC1 ; "PN"]

To a solution of methyl 2-[4-[2-[(10S)-4-(2-hydroxyphenyl)-1,5,6,8,12-pentazatricyclo[8.4.0.02,7]tetradeca-2,4,6-trien-12-yl]acetyl]piperazin-1-yl]spiro[3.3]heptane-6-carboxylate (210 mg, 374 umol, Intermediate PL) in H₂O (3 mL), THF (3 mL), and MeOH (3 mL) was added LiOH·H₂O (62.8 mg, 1.50 mmol). The mixture was stirred at 25° C. for 2 hrs. On completion, the reaction was concentrated under reduced pressure to give the residue. The residue was purified by prep-HPLC (HCl) to give the title compound (130 mg, 58% yield) as a white solid. LC-MS (ESI⁺) m/z 548.4 (M+H)⁺.

### (S)-6-(4-(2-(2-(2-hydroxyphenyl)-6a,7,9,10-tetrahydro-5H-pyrazino[1′,2′:4,5]pyrazino[2,3-c]pyridazin-8(6H)-yl)acetyl)piperazin-1-yl)spiro[3.3]heptane-2-carboxylic acid (Intermediate PO)

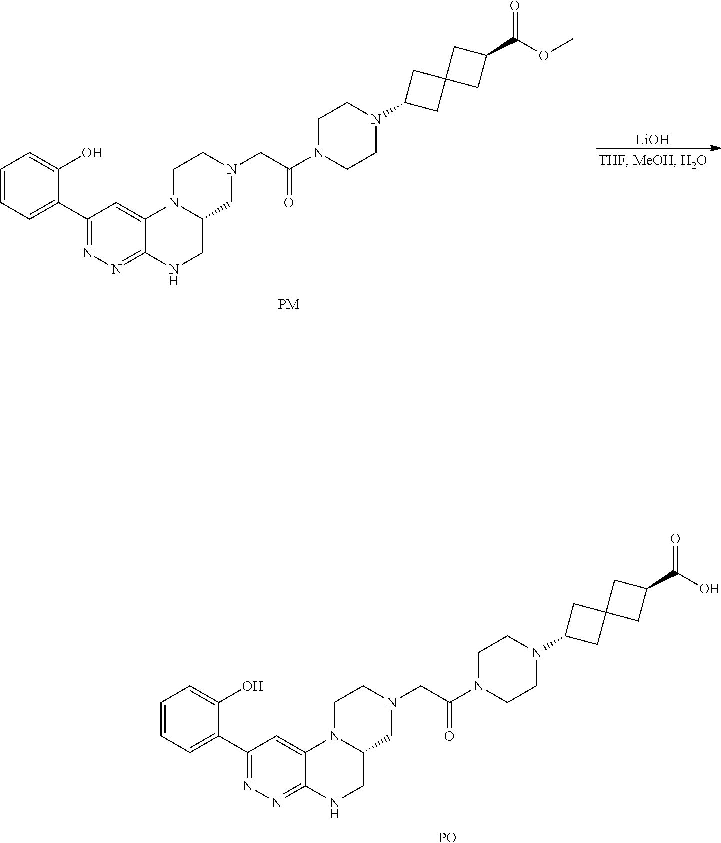 [SMILES: COC(=O)[C@H]1CC2(C1)C[C@H](N1CCN(C(=O)CN3CCN4c5cc(-c6ccccc6O)nnc5NC[C@H]4C3)CC1)C2 ; "LiOH" ; O=C(CN1CCN2c3cc(-c4ccccc4O)nnc3NC[C@H]2C1)N1CCN([C@H]2CC3(C[C@H](C(=O)O)C3)C2)CC1 ; "PO"]

To a solution of methyl 2-[4-[2-[(10S)-4-(2-hydroxyphenyl)-1,5,6,8,12-pentazatricyclo[8.4.0.02,7]tetradeca-2,4,6-trien-12-yl]acetyl]piperazin-1-yl]spiro[3.3]heptane-6-carboxylate (240 mg, 427 umol, Intermediate PM) in H₂O (3 mL), THF (3 mL), and MeOH (3 mL) was added LiOH·H₂O (71.7 mg, 1.71 mmol). The mixture was stirred at 25° C. for 2 hrs. On completion, the reaction mixture was concentrated under reduced pressure to give a residue. The crude product was purified by reversed-phase HPLC (HCl) to give the title compound (160 mg, 64% yield) as a white solid. LC-MS (ESI⁺) m/z 548.3 (M+H)⁺.

### (4-ethynyl-2-fluorophenyl)methanamine (Intermediate PP)

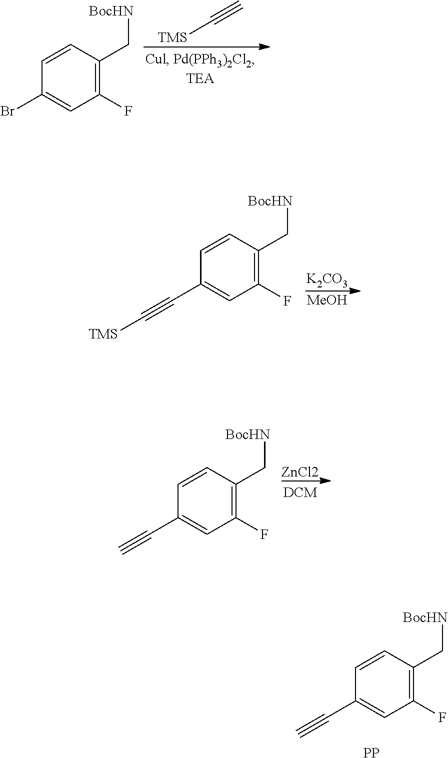

Step 1-tert-butyl 2-fluoro-4-((trimethylsilyl)ethynyl)benzylcarbamate. A mixture of tert-butyl N-[(4-bromo-2-fluoro-phenyl)methyl]carbamate (91 g, 300 mmol, CAS #864262-97-5), ethynyl(trimethyl)silane (176 g, 1.80 mol), CuI (5.70 g, 29.9 mmol), Pd(PPh₃)₂Cl₂ (10.5 g, 15.0 mmol) in TEA (1000 mL) was degassed and purged with N₂ three times. Then the mixture was stirred at 80° C. for 12 hrs under N₂ atmosphere. On completion, the reaction mixture was partitioned between EA (700 mL) and H₂O (700 mL). The organic phase was separated and washed with NaCl (aq) (200 mL×3), dried over Na₂SO₄, filtered and concentrated under reduced pressure to give a residue. The residue was purified by column chromatography (SiO₂, Petroleum ether/Ethyl acetate=20/1 to 5/1) to give the title compound (85 g, 73% yield) as a brown oil. LC-MS (ESI⁺) m/z 266.1 (M−55)⁺.

Step 2-Tert-butyl 4-ethynyl-2-fluorobenzylcarbamate. To a solution of tert-butyl N-[[2-fluoro-4-(2-trimethylsilylethynyl)phenyl]methyl]carbamate (55 g, 171 mmol) in MeOH (600 mL) was added K₂CO₃ (70.9 g, 513 mmol). The mixture was stirred at 25° C. for 2 hrs. On completion, the mixture was filtered to remove the K₂CO₃, saturated NH₄Cl (500 mL) was added and the mixture was extracted with EA (500 mL×2). The combined organic layers was washed with sat. NaCl (300 mL) and dried over Na₂SO₄. The mixture was then filtered and concentrated under reduce pressure to give a residue. The residue was purified by column chromatography (SiO₂, Petroleum ether/Ethyl acetate=10/1 to 1/1) to give the title compound (57 g, 63% yield) as a white solid. LC-MS (ESI⁺) m/z 194.1 (M−55)⁺.

Step 3-(4-Ethynyl-2-fluorophenyl)methanamine. To a solution of tert-butyl N-[(4-ethynyl-2-fluoro-phenyl) methyl] carbamate (10 g, 40.1 mmol) in DCM (100 mL) was added ZnCl₂ (10.9 g, 80.2 mmol, 3.76 mL). The mixture was stirred at 25° C. for 12 hrs. The mixture was then stirred at 25° C. for 12 hrs. On completion, the reaction mixture was filtered to remove the ZnCl₂, and then the solution was concentrated under reduced pressure to give the title compound (21 g) as a yellow solid. LC-MS (ESI⁺) m/z 150.3 (M+H)⁺.

### Phenyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate (Intermediate PQ)

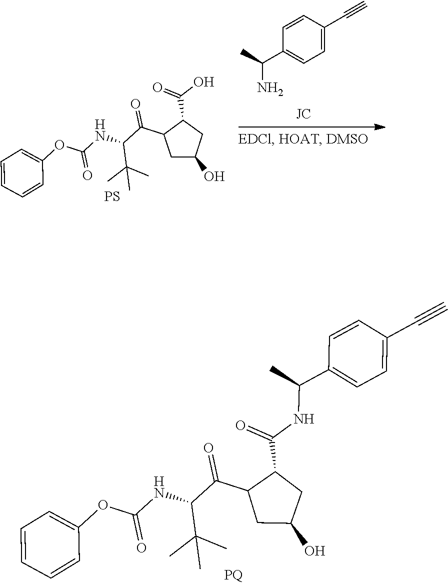

To a solution of (S)-1-(4-ethynylphenyl)ethanamine (1.9 g, 9.9 mmol, Intermediate JC) and (2S,4R)-1-((S)-3,3-dimethyl-2-((phenoxycarbonyl)amino)butanoyl)-4-hydroxypyrrolidine-2-carboxylic acid (3.98 g, 10.9 mmol, Intermediate PS) in DMSO (19 mL) was added EDCI (9 M, 0.4 mL). The mixture was stirred at 25° C. for 30 mins. On completion, the reaction mixture was diluted with H₂O (50 mL) and extracted with EA (50 mL×6). The combined organic layers were dried over Na₂SO₄, filtered and concentrated under reduced pressure to give a residue. The crude product was purified by column chromatography (SiO₂, PE: EA=50/1 to 40/1) to give the title compound (3.47 g, 69% yield) as a rose white solid. LC-MS (ESI+) m/z 492.2 (M+H)⁺.

### (S)-2-(8-(2-(piperidin-4-yl)-2-azaspiro[3.3]heptan-6-yl)-6,6a,7,8,9,10-hexahydro-5H-pyrazino[1′,2′:4,5]pyrazino[2,3-c]pyridazin-2-yl)phenol (Intermediate PR)

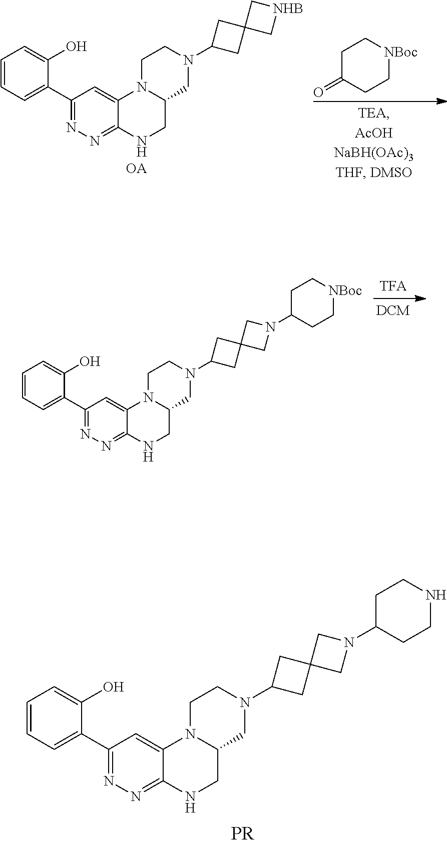

Step 1-(S)-tert-butyl 4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidine-1-carboxylate. To a mixture of (S)-2-(8-(2-azaspiro[3.3]heptan-6-yl)-6,6a,7,8,9,10-hexahydro-5H-pyrazino[1′,2′:4,5]pyrazino[2,3-c]pyridazin-2-yl)phenol (1 g, 2.64 mmol, Intermediate OA) in THF (30 mL) and DMSO (10 mL) was added TEA (802 mg, 7.93 mmol). The mixture was stirred at 25° C. for 1 hr. Then tert-butyl 4-oxopiperidine-1-carboxylate (790 mg, 3.96 mmol, CAS #7909099-07-3) and AcOH (476 mg, 7.93 mmol) was added and the mixture was stirred at 25° C. for 1 hr. Next, NaBH(OAc)₃ (1.40 g, 6.61 mmol) was added at 0° C. and the mixture was stirred at 25° C. for 1 hr. On completion, the reaction mixture was filtered and concentrated under reduced pressure to give a residue. The residue was purified by prep-HPLC (FA condition) to give the title compound (1.2 g, 66% yield, FA) as a yellow solid. LC-MS (ESI+) m/z 562.4 (M−100)⁺.

Step 2-(S)-2-(8-(2-(piperidin-4-yl)-2-azaspiro[3.3]heptan-6-yl)-6,6a,7,8,9,10-hexahydro-5H-pyrazino[1′,2′:4,5]pyrazino[2,3-c]pyridazin-2-yl)phenol. To a mixture of (S)-tert-butyl 4-(6-(2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidine-1-carboxylate (1.1 g, 2.0 mmol) in DCM (12 mL) was added TFA (3.70 g, 32.4 mmol). The mixture was stirred at 25° C. for 1 hr. On completion, the reaction mixture was filtered and concentrated under reduced pressure to give a residue. The residue was purified by prep-HPLC (TFA condition) to give the title compound (834 mg, 71% yield, TFA) as a yellow solid. LC-MS (ESI+) m/z 462.3 (M+H)⁺.

### (2S,4R)-1-((S)-3,3-dimethyl-2-((phenoxycarbonyl)amino)butanoyl)-4-hydroxypyrrolidine-2-carboxylic acid (Intermediate PS)

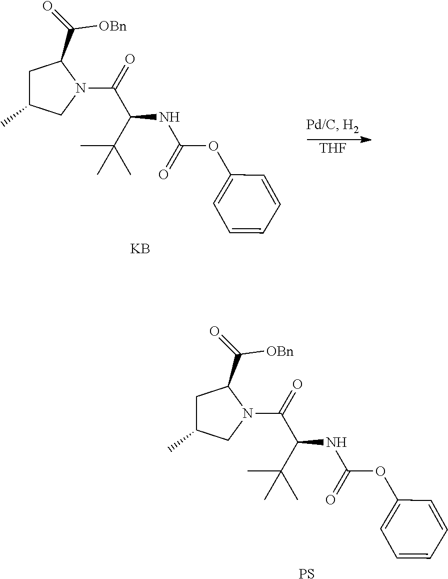 [SMILES: C[C@@H]1C[C@@H](C(=O)OCc2ccccc2)N(C(=O)[C@@H](NC(=O)Oc2ccccc2)C(C)(C)C)C1 ; "KB" ; "THF" ; C[C@@H]1C[C@@H](C(=O)OCc2ccccc2)N(C(=O)[C@@H](NC(=O)Oc2ccccc2)C(C)(C)C)C1 ; "PS"]

To a solution of benzyl (2S,4R)-1-[(2S)-3,3-dimethyl-2-(phenoxycarbonylamino)butanoyl]-4-hydroxy-pyrrolidine-2-carboxylate (600 g, 1.32 mol, Intermediate KB) in THF (7000 mL) was added Pd/C (1.32 mol, 10 wt %). Then the mixture was stirred at 25° C. for 12 hrs under H₂ (45 PSI) atmosphere. On completion, the reaction mixture was filtered carefully and the filtrate was concentrated in vacuo to give the title compound (500 g) as a white solid. LC-MS (ESI⁺) m/z 365.0 (M−NH₂+H)⁺.

### (S)-2-(8-(5-(4,7-diazaspiro[2.5]octan-7-yl)pyrimidin-2-yl)-6,6a,7,8,9,10-hexahydro-5H-pyrazino[1′,2′:4,5]pyrazino[2,3-c]pyridazin-2-yl)phenol (Intermediate PY)

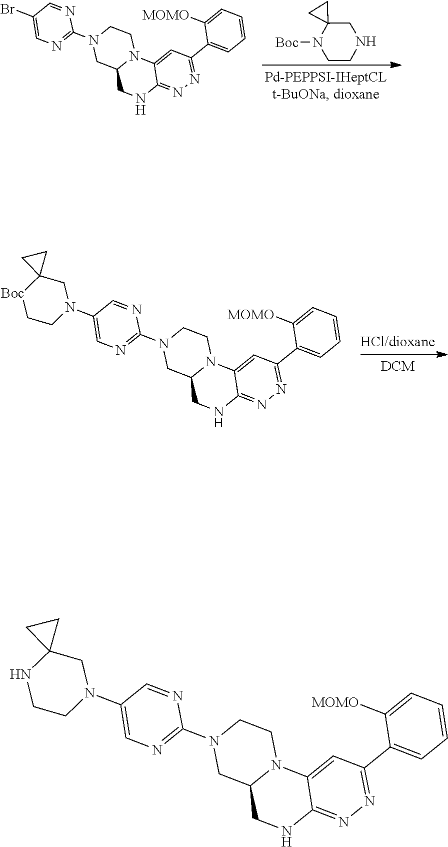

Step 1-(S)-tert-butyl 7-(2-(2-(2-(methoxymethoxy)phenyl)-6a,7,9,10-tetrahydro-5H-pyrazino[1′,2′: 4,5]pyrazino[2,3-c]pyridazin-8(6H)-yl)pyrimidin-5-yl)-4,7-diazaspiro[2.5]octane-4-carboxylate. A mixture of (1OS)-12-(5-bromopyrimidin-2-yl)-4-[2-(methoxymethoxy)phenyl]-1,5,6,8,12-pentazatricyclo[8.4.0.0^{2,7}]tetradeca-2,4,6-triene (1.00 g, 2.06 mmol, synthesized via Step 1 of Intermediate EA), and tert-butyl 4,7-diazaspiro[2.5]octane-4-carboxylate (438 mg, 2.06 mmol, CAS #674792-08-6) in dioxane (25 mL) was degassed and purged with N₂ three times. Then to the mixture was added tBuONa (2 M, 6.19 mL) and 1,3-bis[2,6-bis(1-propylbutyl)phenyl]-4,5-dichloro-2H-imidazol-1-ium-2-ide;3-chloropyridine;dichloropalladium (201 mg, 206 umol) and the mixture was stirred at 120° C. for 12 hrs under N₂ atmosphere. On completion, the aqueous phase was extracted with ethyl acetate (50 mL×3). The combined organic phase was washed with brine (15 mL×2), dried with anhydrous Na₂SO₄, filtered and concentrated in vacuo. The residue was purified by silica gel chromatography (column height: 250 mm, diameter: 100 mm, 100-200 mesh silica gel, DCM/CH₃OH=100/1, 30/1) to afford to afford the title compound (1.2 g, 51% yield) as a white solid. LC-MS (ESI⁺) m/z 616.5 (M+H)⁺.

Step 2-(S)-2-(8-(5-(4,7-diazaspiro[2.5]octan-7-yl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl 7-[2-[(10S)-4-[2-(methoxymethoxy)phenyl]-1,5,6,8,12-pentazatricyclo[8.4.0.0^{2,7}]tetradeca-2,4,6-trien-12-yl]pyrimidin-5-yl]-4,7-diazaspiro[2.5]octane-4-carboxylate (600 mg, 974 umol) in DCM (10 mL) was added HCl/dioxane (4 M, 2.50 mL).The mixture was stirred at 25° C. for 20 min. On completion, the reaction mixture was concentrated under reduced pressure to remove DCM. The crude product was purified by reversed-phase HPLC (0.1% FA condition) to afford the title compound (300 mg, 58% yield, FA) as a yellow solid. LC-MS (ESI⁺) m/z 472.1 (M+H)⁺.

### (S)-ethyl 2-(7-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)-4,7-diazaspiro[2.5]octan-4-yl)spiro[3.5]nonane-7-carboxylate (Intermediate PZ) and (S)-ethyl 2-(7-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)-4,7-diazaspiro[2.5]octan-4-yl)spiro[3.5]nonane-7-carboxylate (Intermediate QA)

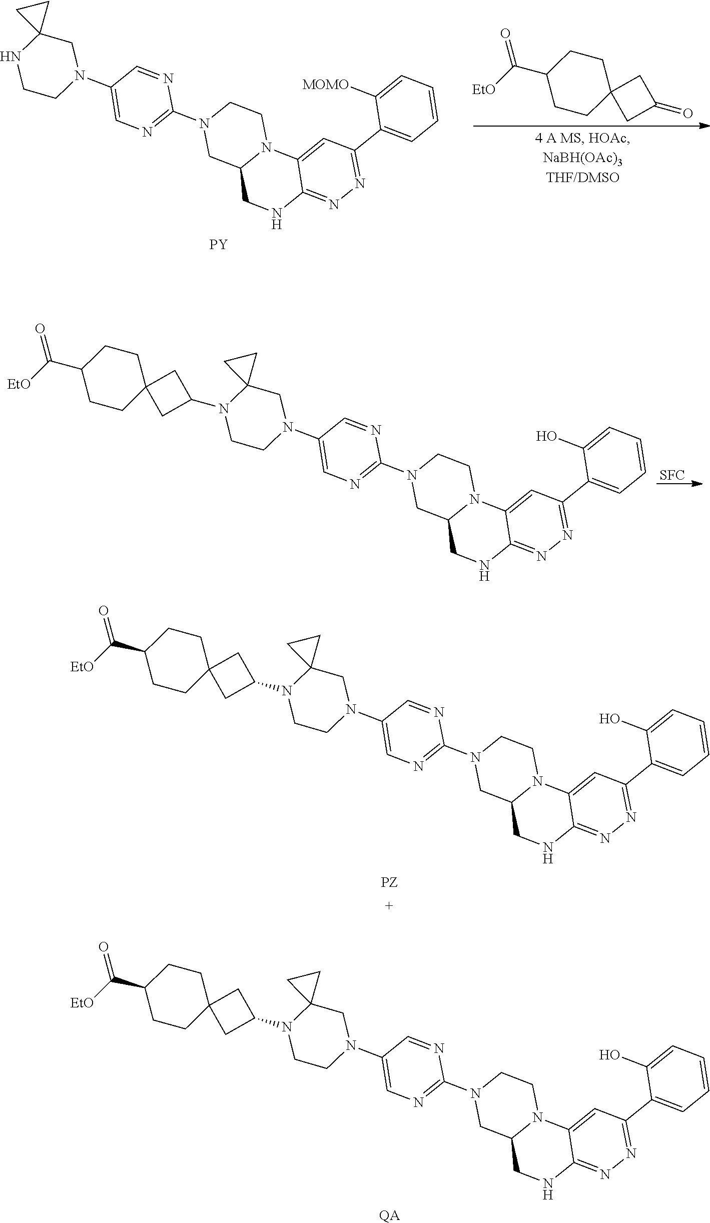

Step 1-(S)-ethyl 2-(7-(2-(2-(2-hydroxyphenyl)-6a,7,9,10-tetrahydro-5H-pyrazino[1′,2′ 4,5]pyrazino[2,3-c]pyridazin-8(6H)-yl)pyrimidin-5-yl)-4,7-diazaspiro[2.5]octan-4-yl)spiro[3.5]nonane-7-carboxylate. To a solution of ethyl 2-oxospiro[3.5]nonane-7-carboxylate (401 mg, 1.91 mmol) in THF (5 mL) and DMSO (5 mL) was added 4 Å molecular sieves (600 mg), HOAc (115 mg, 1.91 mmol) and 2-[(1OS)-12-[5-(4,7-diazaspiro[2.5]octan-7-yl)pyrimidin-2-yl]-1,5,6,8,12-pentazatricyclo[8.4.0.0^{2,7}]tetradeca-2,4,6-trien-4-yl]phenol (300 mg, 636 umol, Intermediate PY) and stirred at 60° C. for 1 hr. Then to the mixture was added NaBH(OAc)₃ (405 mg, 1.91 mmol) and stirred at 0-40° C. for 11 hrs. On completion, the reaction mixture was concentrated under reduced pressure to remove DCM. The crude product was purified by reversed-phase HPLC (0.1% FA condition) to afford the title compound (220 mg, 49% yield) as a white solid. LC-MS (ESI⁺) m/z 666.5 (M+H)⁺.

Step 2-(S)-ethyl 2-(7-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)-4,7-diazaspiro[2.5]octan-4-yl)spiro[3.5]nonane-7-carboxylate and (S)-ethyl 2-(7-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)-4,7-diazaspiro[2.5]octan-4-yl)spiro[3.5]nonane-7-carboxylate. Ethyl 2-[7-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-4,7-diazaspiro[2.5]octan-4-yl]spiro[3.5]nonane-7-carboxylate (220 mg, 330.42 umol) separated by SFC (column: DAICEL CHIRALPAK AD(250 mm*30 mm,10 um);mobile phase: [ACN/IPA(0.1% NH₃H2O)];B %: 50%-50%, A7.7; 100 min) to give the title compounds (100 mg, 44% yield for peak 1 and 130 mg, 54% yield for peak 2) as a white solids. Absolute stereochemistry on the bicycle was assigned arbitrarily. LC-MS (ESI⁺) m/z 666.6 (M+H)⁺ for both isomers.

### (S)-2-(7-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)-4,7-diazaspiro[2.5]octan-4-yl)spiro[3.5]nonane-7-carboxylic acid (Intermediate QB)

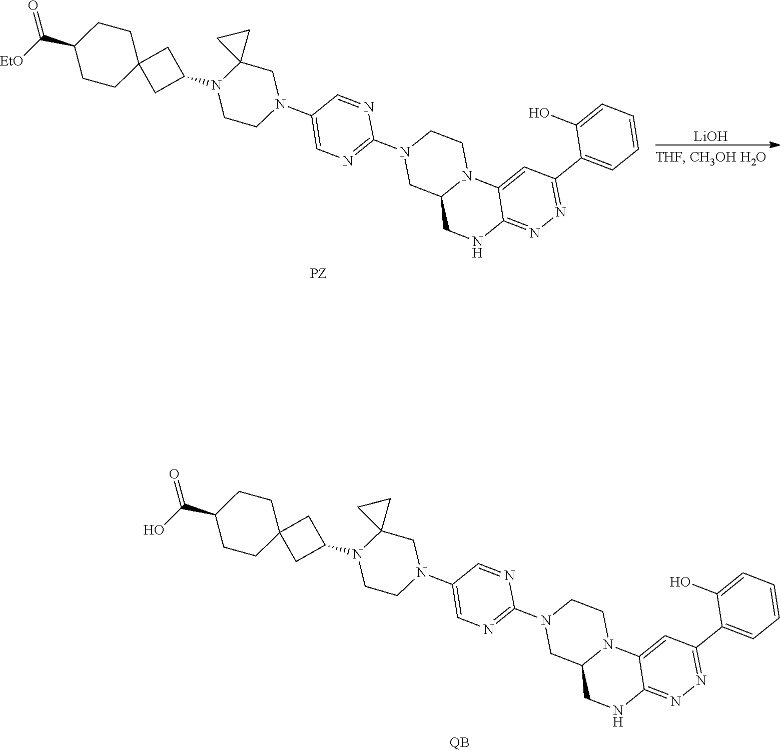

To a solution of ethyl 2-[7-[2-[(1OS)-4-(2-hydroxyphenyl)-1,5,6,8,12-pentazatricyclo[8.4.0.0^{2,7}]tetradeca-2,4,6-trien-12-yl]pyrimidin-5-yl]-4,7-diazaspiro[2.5]octan-4-yl]spiro[3.5]nonane-7-carboxylate (100 mg, 150 umol, Intermediate PZ) in THF (1 mL) and CH₃OH (1 mL) was added LiOH·H₂O (2 M, 1 mL). The mixture was stirred at 25° C. for 1 hrs. On completion, the reaction mixture was adjusted by NaHCO₃ aqueous solution (5 mL) and concentrated under reduced pressure to remove THF. The crude product was purified by reversed-phase HPLC (0.1% FA condition) to give the title compound (60 mg, 61% yield) as a white solid. LC-MS (ESI⁺) m/z 638.5 (M+H)⁺.

### (S)-2-(7-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)-4,7-diazaspiro[2.5]octan-4-yl)spiro[3.5]nonane-7-carboxylic acid (Intermediate QC)

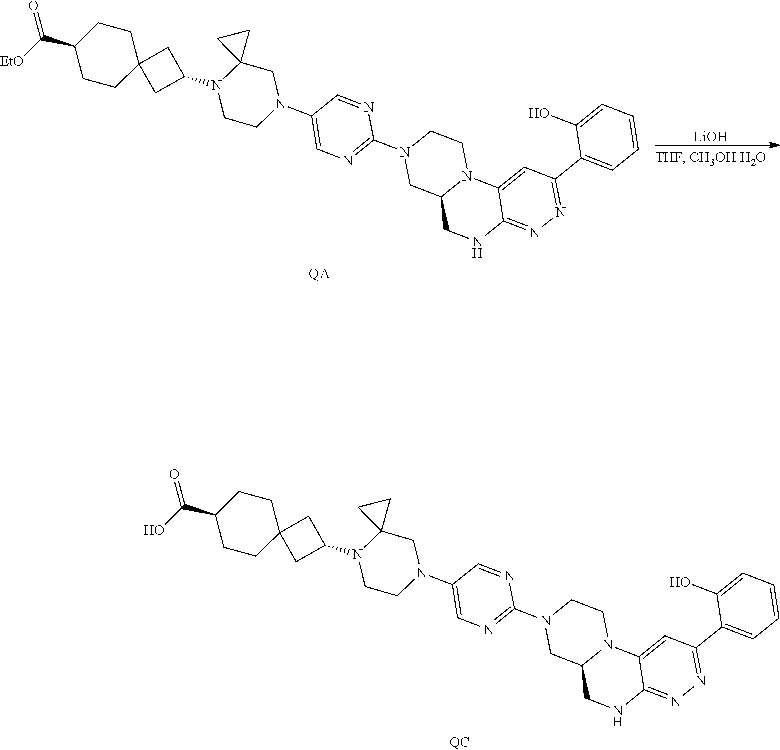

To a solution of ethyl 2-[7-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-4,7-diazaspiro[2.5]octan-4-yl]spiro[3.5]nonane-7-carboxylate (40 mg, 60 umol, Intermediate QA) in THF (1 mL) was added LiOH·H₂O (2 M, 0.5 mL). The mixture was stirred at 25° C. for 1 hr. On completion, the reaction mixture was concentrated under reduced pressure to remove THF. The crude product was purified by reversed-phase HPLC (0.1% FA condition) to give the title compound (20 mg, 50% yield) as a white solid. LC-MS (ESI⁺) m/z 638.5 (M+H)⁺.

### (R)-2-(6-(5-(1-(azetidin-3-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate QD)

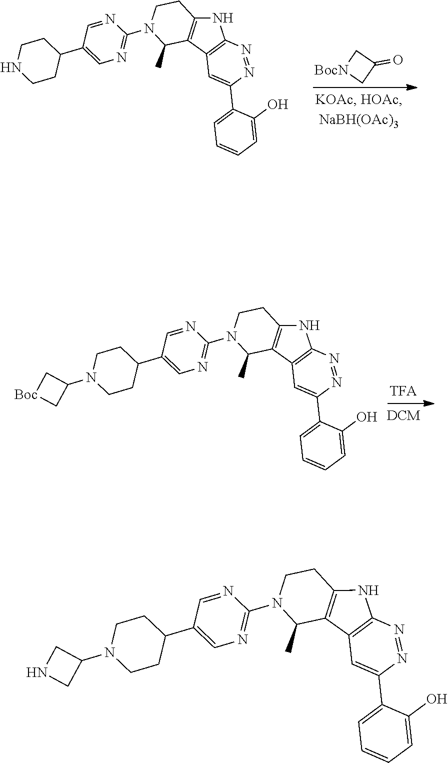

Step 1-(R)-tert-butyl 3-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)azetidine-1-carboxylate. To a solution of 2-[(3R)-3-methyl-4-[5-(4-piperidyl)pyrimidin-2-yl]-4,8,10,11-tetrazatricyclo[7.4.0.0^{2,7}]trideca-1(9),2(7),10,12-tetraen-12-yl]phenol (500 mg, 1.13 mmol, Intermediate B) in THF (8 mL) and DMSO (1.6 mL) was added KOAc (333 mg, 3.40 mmol) and the mixture was stirred at 25° C. for 0.5 hr. Then tert-butyl 3-oxoazetidine-1-carboxylate (252 mg, 1.47 mmol, CAS #398489-26-4) and HOAc (204 mg, 3.40 mmol) was added and the mixture was stirred at 25° C. for 0.5 hr. Next, NaBH(OAc)₃ (600 mg, 2.83 mmol) was added and the mixture was stirred at 25° C. for 3 hrs. The reaction mixture was quenched with MeOH (10 mL) at 25° C. The reaction mixture was concentrated under reduced pressure to remove the THF and MeOH. The crude product was purified by reversed-phase HPLC (0.1% FA condition) to give the title compound (500 mg, 68% yield, FA) as a yellow solid. LC-MS (ESI⁺) m/z 597.3 (M+H)⁺.

Step 2-(R)-2-(6-(5-(1-(azetidin-3-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 3-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]azetidine-1-carboxylate (500 mg, 778 umol) in DCM (5 mL) was added TFA (1.54 g, 13.5 mmol). The mixture was stirred at 25° C. for 1 hr. The reaction mixture was concentrated under reduced pressure give the title compound (480 mg) as a red oil. LC-MS (ESI⁺) m/z 497.2 (M+H)⁺.

### (R)-2-(6-(5-(1-(1-(2-azaspiro[3.3]heptan-6-yl)azetidin-3-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate QE)

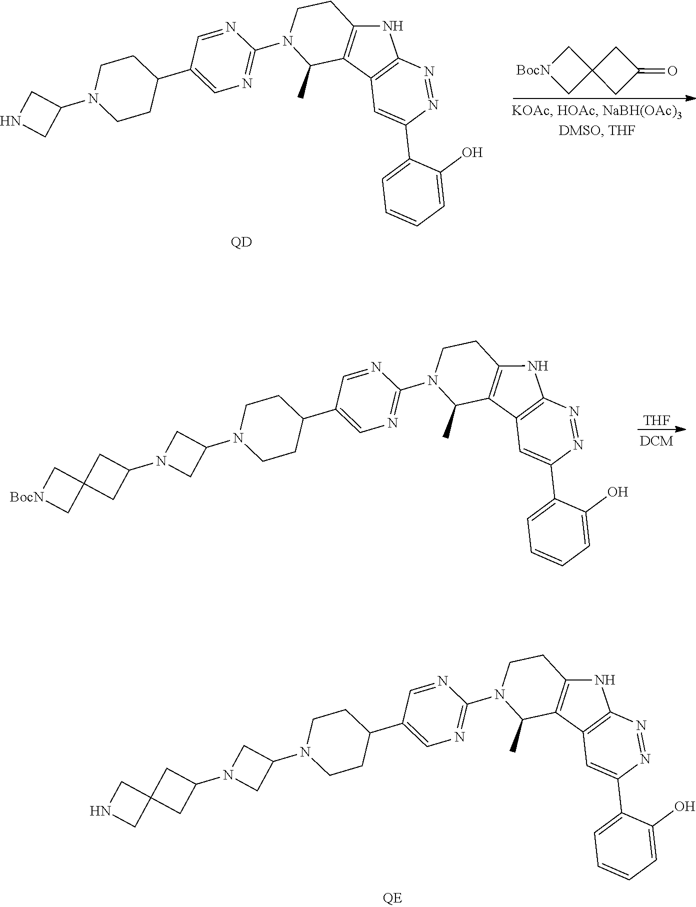

Step 1-(R)-tert-butyl 6-(3-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)azetidin-1-yl)-2-azaspiro[3.3]heptane-2-carboxylate. To a solution of 2-[(3R)-4-[5-[1-(azetidin-3-yl)-4-piperidyl]pyrimidin-2-yl]-3-methyl-4,8,10,11-tetrazatricyclo[7.4.0.0^{2,7}]trideca-1(9),2(7),10,12-tetraen-12-yl]phenol (480 mg, 786 umol, Intermediate QD) in THF (8 mL) was added KOAc (463 mg, 4.72 mmol) and the mixture was stirred at 25° C. for 0.5 hr. Then HOAc (142 mg, 2.36 mmol) and tert-butyl 6-oxo-2-azaspiro[3.3]heptane-2-carboxylate (332 mg, 1.57 mmol) were added and the mixture was stirred at 25° C. for 0.5 hr. Next, NaBH(OAc)₃ (417 mg, 1.97 mmol) was added and the mixture was stirred at 25° C. for 11 hrs. On completion, the reaction mixture was concentrated under reduced pressure to remove THF. The crude product was purified by reversed-phase HPLC (0.1% FA condition) to give the title compound (400 mg, 68% yield) as a yellow solid. LC-MS (ESI⁺) m/z 692.5 (M+H)⁺.

Step 2-(R)-2-(6-(5-(1-(1-(2-azaspiro[3.3]heptan-6-yl)azetidin-3-yl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 6-[3-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]azetidin-1-yl]-2-azaspiro[3.3]heptane-2-carboxylate (100 mg, 135 umol) in DCM (0.5 mL) was added TFA (308 mg, 2.70 mmol). The mixture was stirred at 25° C. for 1 hr. On completion, the reaction mixture was concentrated under reduced pressure to give the title compound (100 mg) as a yellow oil. LC-MS (ESI⁺) m/z 592.4 (M+H)⁺.

### (S)-2-(8-(5-(4-(piperazin-1-ylmethyl)phenyl)pyrimidin-2-yl)-6,6a,7,8,9,10-hexahydro-5H-pyrazino[1′,2′:4,5]pyrazino[2,3-c]pyridazin-2-yl)phenol (Intermediate QF)

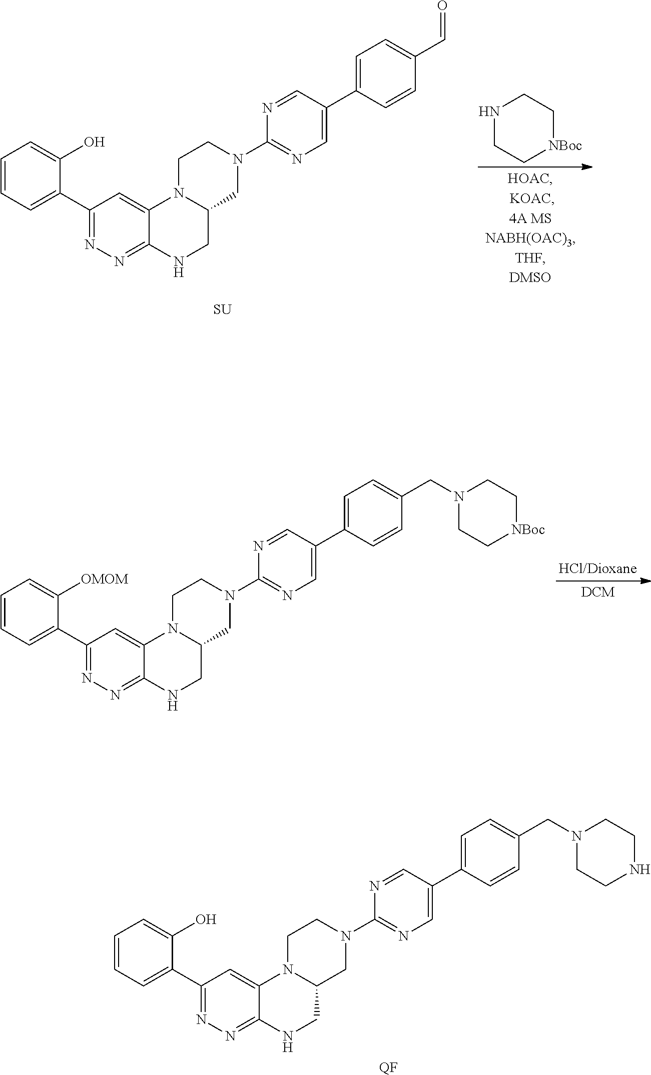

Step 1-(S)-tert-butyl 4-(4-(2-(2-(2-(methoxymethoxy)phenyl)-6a,7,9,10-tetrahydro-5H-pyrazino[1′,2′:4,5]pyrazino[2,3-c]pyridazin-8(6H)-yl)pyrimidin-5-yl)benzyl)piperazine-1-carboxylate. To a solution of 4-[2-[(1OS)-4-[2-(methoxymethoxy)phenyl]-1,5,6,8,12-pentazatricyclo[8.4.0.0^{2,7}]tetradeca-2,4,6-trien-12-yl]pyrimidin-5-yl]benzaldehyde (200 mg, 392 umol, Intermediate SU) in THF (1 mL) and DMSO (2 mL) was added tert-butyl piperazine-1-carboxylate (87.7 mg, 471 umol, CAS #143238-38-4, HOAc (70.7 mg, 1.18 mmol), 4 Å molecular sieves (20 mg, 3.93 mmol) and KOAC (115 mg, 1.18 mmol). The mixture was stirred at 0° C. for 0.5 hour, then NaBH(OAc)₃ (249 mg, 1.18 mmol) was added and the mixture was stirred at 40° C. for 11.5 hours. On completion, the reaction mixture was filtered and concentrated under reduced pressure to give a residue. The residue was purified by reversed-phase HPLC (0.1% FA condition) to give the title compound (120 mg, 44% yield) as a white solid. LC-MS (ESI⁺) m z 680.3 (M+H)⁺.

Step 2-(S)-2-(8-(5-(4-(piperazin-1-ylmethyl)phenyl)pyrimidin-2-yl)-6,6a,7,8,9,10-hexahydro-5H-pyrazino[1′,2′:4,5]pyrazino[2,3-c]pyridazin-2-yl)phenol. To a solution of tert-butyl 4-[[4-[2-[(10S)-4-[2-(methoxymethoxy)phenyl]-1,5,6,8,12-pentazatricyclo[8.4.0.0^{2,7}]tetradeca-2,4,6-trien-12-yl]pyrimidin-5-yl]phenyl]methyl]piperazine-1-carboxylate (120 mg, 176 umol) in DCM (10 mL) was added HCl/dioxane (4 M, 441 uL). The mixture was stirred at 25° C. for 1 hr. On completion, the reaction mixture was filtered and concentrated under reduced pressure to give the title compound (160 mg, HCl salt) as a white solid. LC-MS (ESI⁺) m/z 535.8 (M+H)⁺.

### (4-Ethynyl-2-methylphenyl)methanamine (Intermediate QG)

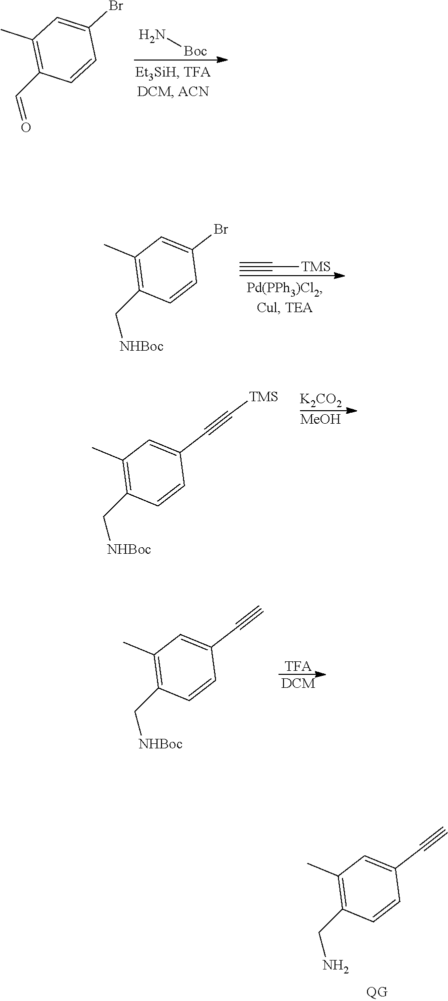

Step 1-Tert-butyl 4-bromo-2-methylbenzylcarbamate. To a solution of 4-bromo-2-methylbenzaldehyde (8.83 g, 75.4 mmol, CAS #4248-19-5) and 4-bromo-2-methyl-benzaldehyde (5 g, 25.1 mmol) in DCM (20 mL) and ACN (60 mL) was added Et₃SiH (8.76 g, 75.4 mmol, 12.0 mL) and TFA (5.73 g, 50.2 mmol, 3.72 mL) at 25° C., then the mixture was stirred at 25° C. for 12 hrs. On completion, the reaction mixture was quenched with water (40 mL) and extracted by ethyl acetate (30×3 mL). The extracts were washed by brine (50 mL) and dried over anhydrous sodium sulfate, filtered and concentrated in vacuo to give the crude residue. The residue was purified by column chromatography (SiO₂, Petroleum ether/Ethyl acetate=10/1 to 5/1) to give the title compound (7.5 g, 93% yield) as yellow oil. LC-MS (ESI⁺) m/z 244.0. (M+H)⁺.

Step 2-Tert-butyl 2-methyl-4-((trimethylsilyl)ethynyl)benzylcarbamate. To a solution of tert-butyl 4-bromo-2-methylbenzylcarbamate (6.54 g, 66.6 mmol, 9.23 mL) and tert-butyl N-[(4-bromo-2-methyl-phenyl)methyl]carbamate (2 g, 6.66 mmol) in TEA (20 mL) was added Pd(PPh₃)₂Cl₂ (468 mg, 666 umol), and CuI (254 mg, 1.33 mmol) at 25° C. Then the mixture was stirred at 80° C. for 12 hrs. On completion, the reaction mixture was filtered and concentrated in vacuo to give the crude residue. The residue was purified by column chromatography (SiO₂, Petroleum ether/Ethyl acetate=10/1) to give the title compound (1.7 g, 79% yield) as a yellow oil. LC-MS (ESI⁺) m/z 262.0. (M+H)⁺.

Step 3-Tert-butyl 4-ethynyl-2-methylbenzylcarbamate. To a solution tert-butyl 2-methyl-4-((trimethylsilyl)ethynyl)benzylcarbamate (200 mg, 630 umol) in MeOH (2 mL) was added K₂CO₃ (87.1 mg, 630 umol) at 25° C., then the mixture was stirred at 25° C. for 1 hr. On completion, the reaction mixture was quenched with water (2 mL) and extracted by ethyl acetate (2×3 mL). The extracts were washed by brine (3 mL) and dried over anhydrous sodium sulfate, filtered and concentrated in vacuo to give the title compound (130 mg) as a yellow solid. LC-MS (ESI⁺) m/z 190.1. (M+H)⁺.

Step 4-(4-Ethynyl-2-methylphenyl)methanamine. To a solution of tert-butyl 4-ethynyl-2-methylbenzylcarbamate (130 mg, 530 umol) in DCM (1 mL) was added TFA (400 mg, 3.51 mmol, 260 uL) at 25° C., then the mixture was stirred at 25° C. for 2 hrs. On completion, the reaction mixture was filtered and concentrated in vacuo to give the title compound (100 mg) as a yellow oil. LC-MS (ESI⁺) m z 129.2. (M+H)⁺.

### (2-methoxy-4-(prop-1-yn-1-yl)phenyl)methanamine (Intermediate QH)

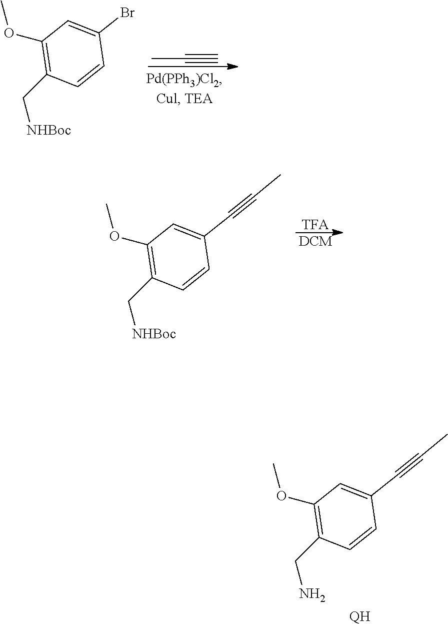

Step 1-Tert-butyl 2-methoxy-4-(prop-1-yn-1-yl)benzylcarbamate. To a solution of tert-butyl 4-bromo-2-methoxybenzylcarbamate (1 g, 3.16 mmol, synthesized via Steps 1 of Intermediate HO) and prop-1-yne (2 M, 12.7 mL) in TEA (10 mL) was added CuI (120 mg, 632 umol) and Pd(PPh₃)₂Cl₂ (222 mg, 316 umol) at 25° C., then the mixture was stirred at 80° C. for 12 hrs. On completion, the reaction mixture was filtered and concentrated in vacuo to give the crude residue. The residue was purified by column chromatography (SiO₂, Petroleum ether/Ethyl acetate=9/1) to give the title compound (600 mg, 67% yield) as colorless oil. LC-MS (ESI⁺) m/z 159.3. (M+H)⁺.

Step 2-(2-methoxy-4-(prop-1-yn-1-yl)phenyl)methanamine. To a solution of tert-butyl 2-methoxy-4-(prop-1-yn-1-yl)benzylcarbamate (50 mg, 181 umol) in DCM (1 mL) was added TFA (462 mg, 4.05 mmol) at 25° C., then the mixture was stirred at 25° C. for 1 hr. On completion, the reaction mixture was filtered and concentrated in vacuo to give the title compound (30 mg) as yellow solid. LC-MS (ESI⁺) m/z 159.1. (M+H)⁺.

### (2S,4R)—N—((S)-1-(4-ethynylphenyl)ethyl)-4-hydroxypyrrolidine-2-carboxamide (Intermediate QI)

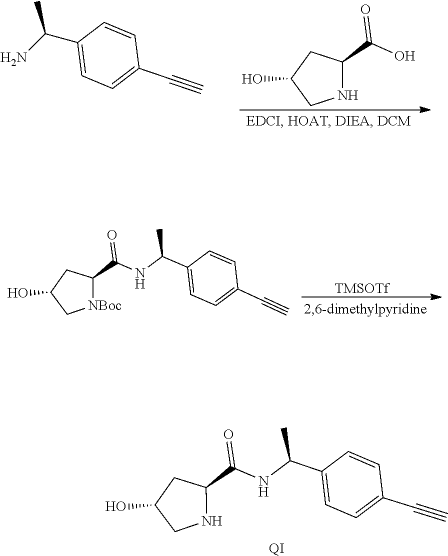

Step 1-(2S,4R)-tert-butyl 2-(((S)-1-(4-ethynylphenyl)ethyl)carbamoyl)-4-hydroxypyrrolidine-1-carboxylate. To a solution of (1S)-1-(4-ethynylphenyl)ethanamine (23.8 g, 163 mmol, CAS #630421-46-4), and (2S,4R)-1-tert-butoxycarbonyl-4-hydroxy-pyrrolidine-2-carboxylic acid (45.5 g, 196 mmol, CAS #51-35-4) in DCM (280 mL) was added DIEA (84.7 g, 655 mmol), EDCI (47.1 g, 245 mmol) and HOAt (33.4 g, 245 mmol). The mixture was stirred at 25° C. for 2 hrs. On completion, the reaction mixture was extracted with DCM (300 mL×3). The combined organic layers were washed with brine (100 mL×2), dried over Na₂SO₄, filtered and concentrated under reduced pressure to give a residue. The residue was purified by prep-HPLC (FA condition) to give the title compound (15.2 g, 22% yield, FA) as a yellow solid. LC-MS (ESI⁺) m/z 303.0 (M−55)⁺.

Step 2-(2S,4R)—N—((S)-1-(4-ethynylphenyl)ethyl)-4-hydroxypyrrolidine-2-carboxamide. To a solution of tert-butyl (2S,4R)-2-[[(1S)-1-(4-ethynylphenyl)ethyl]carbamoyl]-4-hydroxy-pyrrolidine-1-carboxylate (15.2 g, 37.58 mmol) in DCM (70 mL) was added trimethylsilyl trifluoromethanesulfonate (33.4 g, 150 mmol) and 2,6-dimethyl(¹⁵N)pyridine (32.5 g, 300 mmol). The mixture was stirred at 25° C. for 1 hr. On completion, the reaction mixture was filtered and concentrated under reduced pressure to give a residue. The residue was purified by prep-HPLC (FA condition) and (NH₃.H₂O condition) to give the title compound (1 g, 10% yield,) as a white solid. LC-MS (ESI⁺) m/z 259.1 (M+H)⁺.

### Ethyl 2-(3-(2-bromoethoxy)isoxazol-5-yl)-3-methylbutanoate (Intermediate QJ)

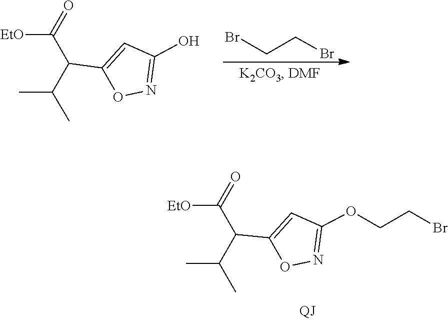

To a solution of ethyl 2-(3-hydroxyisoxazol-5-yl)-3-methylbutanoate (2 g, 9.38 mmol, synthesized via Steps 1-4 of Intermediate IH) in DMF (30 mL) was added K₂CO₃ (5.19 g, 37.5 mmol) and 1,2-dibromoethane (4.41 g, 23.5 mmol). The mixture was stirred at 40° C. for 2 hrs. On completion, the reaction mixture was concentrated in vacuo to give a residue. The crude product was purified by column chromatography (SiO₂, Petroleum ether/Ethyl acetate=15/1) to give the title compound (1.8 g, 58% yield) as a white solid. LC-MS (ESI⁺) m/z 321.9 (M+H)⁺.

### 2-(3-(2-((S)-2-(2-hydroxyphenyl)-6a,7,9,10-tetrahydro-5H-pyrazino[1′,2′:4,5]pyrazino[2,3-c]pyridazin-8(6H)-yl)ethoxy)isoxazol-5-yl)-3-methylbutanoic acid (Intermediate QK)

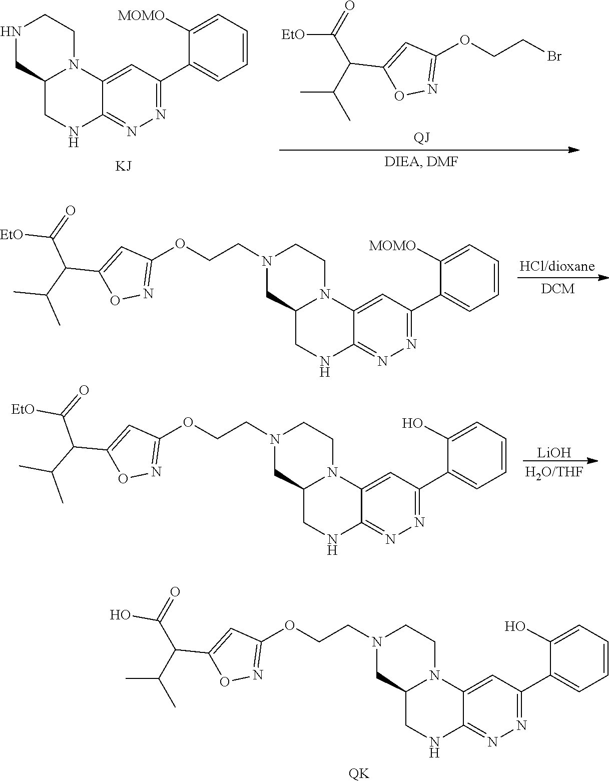

Step 1-Ethyl 2-(3-(2-((S)-2-(2-(methoxymethoxy)phenyl)-6a,7,9,10-tetrahydro-5H-pyrazino[1′,2′:4,5]pyrazino[2,3-c]pyridazin-8(6H)-yl)ethoxy)isoxazol-5-yl)-3-methylbutanoate. To a mixture of ethyl 2-(3-(2-bromoethoxy)isoxazol-5-yl)-3-methylbutanoate (500 mg, 1.56 mmol, Intermediate QJ) in DMF (5 mL) was added DIEA (807 mg, 6.25 mmol) and (R)-2-(2-(methoxymethoxy)phenyl)-6,6a,7,8,9,10-hexahydro-5H-pyrazino[1′,2′:4,5]pyrazino[2,3-c]pyridazine (1.02 g, 3.12 mmol, Intermediate KJ). The mixture was then stirred at 60° C. for 12 hrs. On completion, the reaction mixture was concentrated in vacuo to give a residue. The crude product was purified by column chromatography (SiO₂, Petroleum ether/Ethyl acetate=3/1) to give the title compound (900 mg, 85% yield) as a yellow oil. LC-MS (ESI⁺) m z 567.4 (M+H)⁺.

Step 2-Ethyl 2-(3-(2-((S)-2-(2-hydroxyphenyl)-6a,7,9,10-tetrahydro-5H-pyrazino[1′,2′:4,5] pyrazino[2,3-c]pyridazin-8(6H)-yl)ethoxy)isoxazol-5-yl)-3-methylbutanoate. To a solution of ethyl 2-(3-(2-((S)-2-(2-(methoxymethoxy)phenyl)-6a,7,9,10-tetrahydro-5H-pyrazino[1′,2′:4,5]pyrazino[2,3-c]pyridazin-8(6H)-yl)ethoxy)isoxazol-5-yl)-3-methylbutanoate (400 mg, 705.91 umol) in DCM (4 mL) was added HCl/dioxane (4 M, 2 mL). The mixture was then stirred at 25° C. for 2 hrs. On completion, the reaction mixture was concentrated in vacuo to give the title compound (300 mg, crude) as a white solid. LC-MS (ESI⁺) m/z 529.4 (M+H)⁺.

Step 3-2-(3-(2-((S)-2-(2-hydroxyphenyl)-6a,7,9,10-tetrahydro-5H-pyrazino[1′,2′:4,5]pyrazino[2,3-c]pyridazin-8(6H)-yl)ethoxy)isoxazol-5-yl)-3-methylbutanoic acid. To a solution of ethyl 2-(3-(2-((S)-2-(2-hydroxyphenyl)-6a,7,9,10-tetrahydro- 5H-pyrazino[1′,2′:4,5]pyrazino[2,3-c]pyridazin-8(6H)-yl)ethoxy)isoxazol-5-yl)-3-methylbutanoate (300 mg, 574 umol) in THF (5 mL) and H₂O (5 mL) was added LiOH (41.2 mg, 1.72 mmol). The mixture was then stirred at 25° C. for 2 hrs. On completion, the reaction mixture was concentrated in vacuo to give a residue. The crude product was purified by reversed-phase HPLC(0.1% NH3•H₂O) to give the title compound (70 mg, 23% yield) as a white solid. LC-MS (ESI⁺) m/z 495.1 (M+H)⁺.

### (S)-3-(2-(2-hydroxyphenyl)-6a,7,9,10-tetrahydro-5H-pyrazino[1′,2′:4,5]pyrazino[2,3-c]pyridazin-8(6H)-yl)propanoic acid (Intermediate QL)

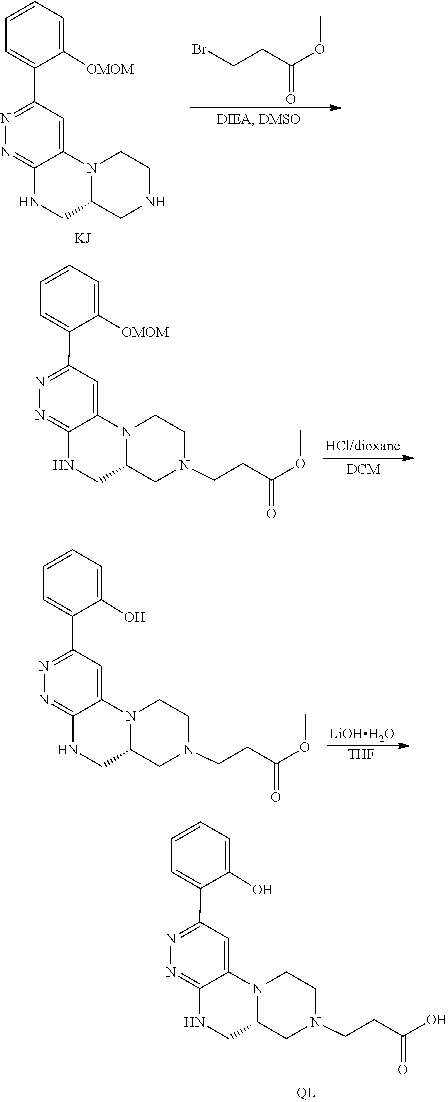

Step 1-(S)-methyl 3-(2-(2-(methoxymethoxy)phenyl)-6a,7,9,10-tetrahydro-5H-pyrazino[1′,2′:4,5]pyrazino[2,3-c]pyridazin-8(6H)-yl)propanoate. To a solution of (1OR)-4-[2-(methoxymethoxy)phenyl]-1,5,6,8,12-pentazatricyclo[8.4.0.0^{2,7}]tetradeca-2,4,6-triene (1 g, 3.05 mmol, Intermediate KJ) in DMSO (15 mL) was added DIEA (1.18 g, 9.16 mmol) and methyl 3-bromopropanoate (663 mg, 3.97 mmol, CAS #23680-40-2). The mixture was stirred at 40° C. for 1 hr. On completion, the reaction mixture was purified directly by reversed-phase HPLC (0.1% FA condition) to give the title compound (800 mg, 47% yield) as a yellow solid. LC-MS (ESI⁺) m/z 414.1 (M+H)⁺.

Step 2-(S)-methyl 3-(2-(2-hydroxyphenyl)-6a,7,9,10-tetrahydro-5H-pyrazino[1′,2′:4,5]pyrazino[2,3-c]pyridazin-8(6H)-yl)propanoate. To a solution of methyl 3-[(10S)-4-[2-(methoxymethoxy)phenyl]-1,5,6,8,12-pentazatricyclo[8.4.0.02,7]tetradeca-2,4,6-trien-12-yl]propanoate (700 mg, 1.69 mmol) in DCM (14 mL) was added HCl/dioxane (4 M, 7.00 mL). The mixture was then stirred at 25° C. for 30 mins. The reaction mixture was concentrated under reduced pressure give the title compound (680 mg) as a white solid. LC-MS (ESI⁺) m/z 370.2 (M+H)⁺.

Step 3-(S)-3-(2-(2-hydroxyphenyl)-6a,7,9,10-tetrahydro-5H-pyrazino[1′,2′:4,5]pyrazino[2,3-c]pyridazin-8(6H)-yl)propanoic acid. To a solution of methyl 3-(10S)-4-(2-hydroxyphenyl)-1,5,6,8,12-pentazatricyclo[8.4.0.02,7]tetradeca-2,4,6-trien-12-yl]propanoate (580 mg, 1.43 mmol) in THF (6 mL) was added LiOH·H₂O (2 M, 2.90 mL). The mixture was then stirred at 25° C. for 1 hr. On completion, the reaction mixture was concentrated under reduced pressure. The crude product was purified by reversed-phase HPLC (0.1% HCl condition) to give the title compound (100 mg, 20% yield) as a white solid. LC-MS (ESI⁺) m/z 356.1 (M+H)⁺.

### Benzyl 4-(6-(methoxycarbonyl)spiro[3.3]heptan-2-yl)piperazine-1-carboxylate (Intermediate QM) and benzyl 4-(6-(methoxycarbonyl)spiro[3.3]heptan-2-yl)piperazine-1-carboxylate (Intermediate QN)

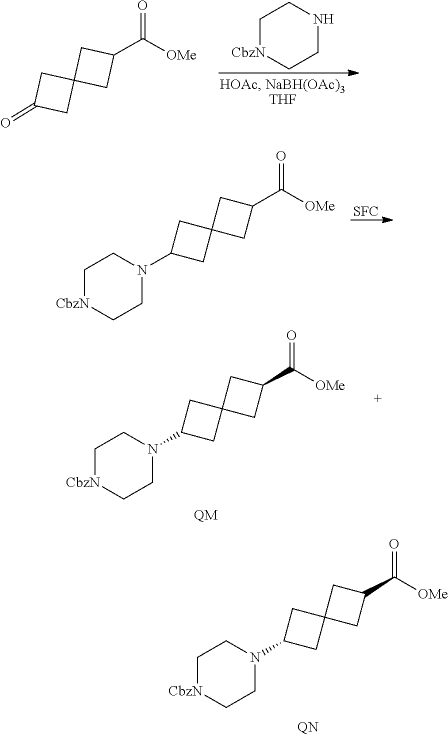

Step 1-Benzyl 4-(6-(methoxycarbonyl)spiro[3.3]heptan-2-yl)piperazine-1-carboxylate. To a solution of benzyl piperazine-1-carboxylate (3 g, 13.6 mmol, CAS #31166-44-6) and methyl 2-oxospiro[3.3]heptane-6-carboxylate (2.29 g, 13.6 mmol, CAS #1138480-98-4) in THF (30 mL) was added HOAc (2.45 g, 40.9 mmol), then the mixture was stirred at 25° C. for 30 mins. Then NaBH(OAc)₃ (8.66 g, 40.9 mmol) was added and the mixture was stirred at 25° C. for 3.5 hrs. On completion, the reaction mixture was quenched with NaHCO₃ 40 mL at 25° C., and then diluted with ethyl acetate (100 mL) and extracted with water (40 mL×2). The combined organic layers were washed with brine (40 mL×2), dried over Na₂SO₄, filtered and concentrated under reduced pressure to give a residue. The residue was purified by column chromatography (SiO₂, DCM/MeOH=50/1 to 20/1) to give the title compound (3.6 g, 67% yield) as a yellow oil. LC-MS (ESI⁺) m/z 373.6 (M+H)⁺.

Step 2-Benzyl 4-(6-(methoxycarbonyl)spiro[3.3]heptan-2-yl)piperazine-1-carboxylate and benzyl 4-(6-(methoxycarbonyl)spiro[3.3]heptan-2-yl)piperazine-1-carboxylate. Benzyl 4-(6-methoxycarbonylspiro[3.3]heptan-2-yl)piperazine-1-carboxylate (3 g, 8.05 mmol) was separated by SFC (column: DAICEL CHIRALPAK AD (250 mm×30 mm, 10 um); mobile phase: [0.1% NH₃H2O MEOH]; B %: 40%-40%, A7.2; 432 min) to give peak 1 benzyl 4-(6-methoxycarbonylspiro[3.3]heptan-2-yl)piperazine-1-carboxylate (1.6 g) as a yellow solid (LC-MS (ESI⁺) m/z 373.3 (M+H)⁺) and peak 2 benzyl 4-(6-methoxycarbonylspiro[3.3]heptan-2-yl)piperazine-1-carboxylate (1.6 g) as a yellow solid (LC-MS (ESI⁺) m/z 373.6 (M+H)⁺). Absolute stereochemistry of the spirocycles was assigned arbitrarily.

### Methyl 6-(piperazin-1-yl)spiro[3.3]heptane-2-carboxylate (Intermediate QO)

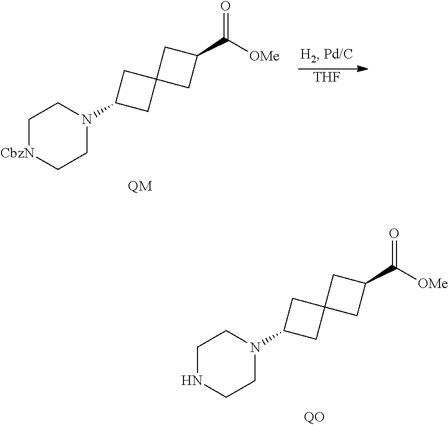

To a solution of benzyl 4-(6-methoxycarbonylspiro[3.3]heptan-2-yl)piperazine-1-carboxylate (500 mg, 1.34 mmol, Intermediate QM) in THF (10 mL) was added Pd/C (10%, 0.5 g) under N₂ atmosphere. The suspension was degassed and purged with H₂ 3 times. Then the mixture was stirred under H₂ (15 Psi) at 25° C. for 12 hrs. On completion, the reaction mixture was filtered and concentrated under reduced pressure to give the title compound (310 mg) as a brown oil. LC-MS (ELSD) m/z 239.3 (M+H)⁺.

### Methyl 6-(piperazin-1-yl)spiro[3.3]heptane-2-carboxylate (Intermediate QP)

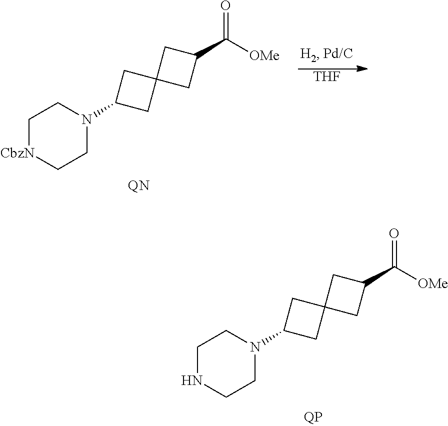

To a solution of benzyl 4-(6-methoxycarbonylspiro[3.3]heptan-2-yl)piperazine-1-carboxylate (500 mg, 1.34 mmol, Intermediate QN) in THF (10 mL) was added Pd/C (10 wt %, 0.5 g) under N₂ atmosphere. The suspension was degassed and purged with H₂ 3 times. Then the mixture was stirred under H₂ (15 Psi) at 25° C. for 12 hrs. On completion, the reaction mixture was filtered and concentrated under reduced pressure to give the title compound (300 mg) as a yellow solid. LC-MS (ELSD) m/z 239.3 (M+H)⁺.

### (S)-6-(4-(3-(2-(2-hydroxyphenyl)-6a,7,9,10-tetrahydro-5H-pyrazino[1′,2′:4,5]pyrazino[2,3-c]pyridazin-8(6H)-yl)propanoyl)piperazin-1-yl)spiro[3.3]heptane-2-carboxylic acid (Intermediate QQ)

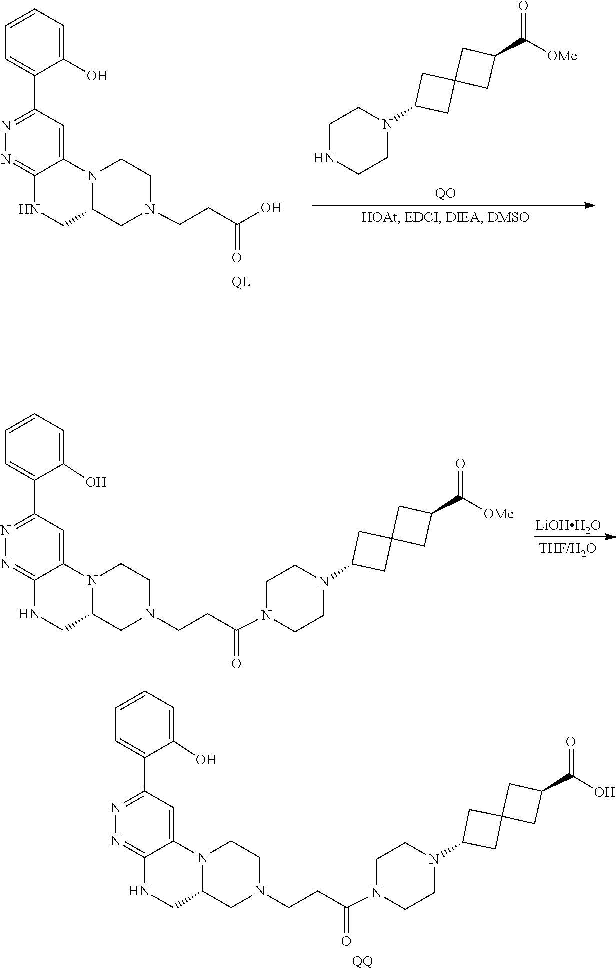

Step 1-(S)-methyl 6-(4-(3-(2-(2-hydroxyphenyl)-6a,7,9,10-tetrahydro-5H-pyrazino[1′,2′:4,5]pyrazino[2,3-c]pyridazin-8(6H)-yl)propanoyl)piperazin-1-yl)spiro[3.3]heptane-2-carboxylate. To a solution of 3-[(10S)-4-(2-hydroxyphenyl)-1,5,6,8,12-pentazatricyclo[8.4.0.0^{2,7}]tetradeca-2,4,6-trien-12-yl]propanoic acid (70 mg, 197 umol, Intermediate QL) and methyl 2-piperazin-1-ylspiro[3.3]heptane-6-carboxylate (70.4 mg, 295 umol, Intermediate QO) in DMSO (2 mL) was added EDCI (56.6 mg, 295 umol), HOAt (40.2 mg, 295 umol) and DIEA (127 mg, 985 umol). The mixture was then stirred at 25° C. for 12 hrs. The reaction mixture was purified directly by reversed-phase HPLC (0.1% FA condition) to give the title compound (100 mg, 82% yield) as a white solid. LC-MS (ESI⁺) m/z 576.4 (M+H)⁺.

Step 2-(S)-6-(4-(3-(2-(2-hydroxyphenyl)-6a,7,9,10-tetrahydro-5H-pyrazino[1′,2′:4,5]pyrazino[2,3-c]pyridazin-8(6H)-yl)propanoyl)piperazin-1-yl)spiro[3.3]heptane-2-carboxylic acid. To a solution of methyl 2-[4-[3-[(10S)-4-(2-hydroxyphenyl)-1,5,6,8,12-pentazatricyclo[8.4.0.0²′7]tetradeca-2,4,6-trien-12-yl]propanoyl]piperazin-1-yl]spiro[3.3]heptane-6-carboxylate (110 mg, 191 umol) in THF (1 mL) was added LiOH·H₂O (2 M, 0.5 mL). The mixture was then stirred at 25° C. for 1 hr. On completion, the reaction mixture was filtered and concentrated under reduced pressure to give a residue. The crude product was purified by reversed-phase HPLC (0.1% HCl condition) to give the title compound (30 mg, 25% yield) as a white solid. LC-MS (ESI⁺) m/z 562.3 (M+H)⁺.

### (S)-6-(4-(3-(2-(2-hydroxyphenyl)-6a,7,9,10-tetrahydro-5H-pyrazino[1′,2′:4,5]pyrazino[2,3-c]pyridazin-8(6H)-yl)propanoyl)piperazin-1-yl)spiro[3.3]heptane-2-carboxylic acid (Intermediate QR)

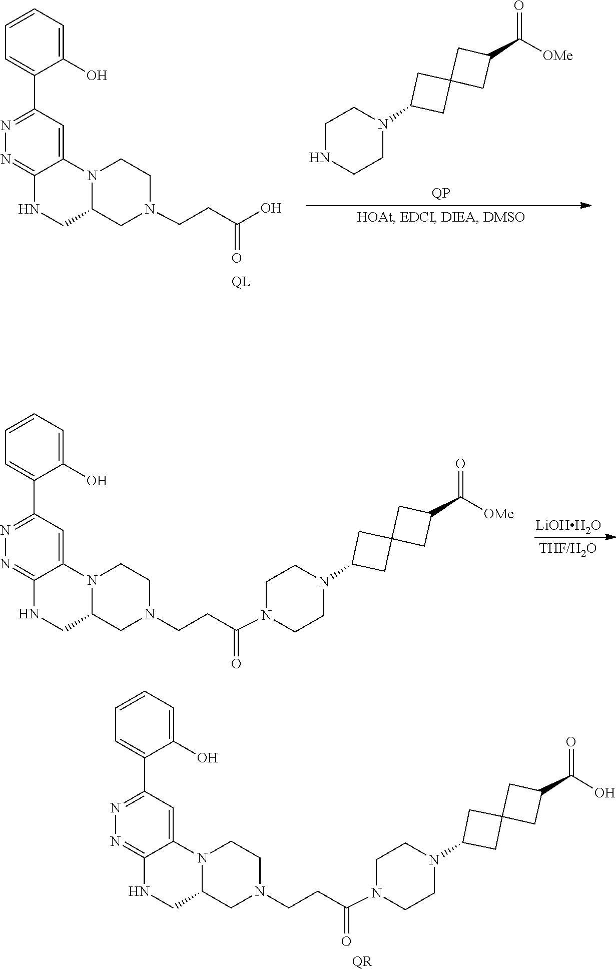

Step 1-(S)-methyl 6-(4-(3-(2-(2-hydroxyphenyl)-6a,7,9,10-tetrahydro-5H-pyrazino[1′,2′: 4,5]pyrazino[2,3-c]pyridazin-8(6H)-yl)propanoyl)piperazin-1-yl)spiro[3.3]heptane-2-carboxylate. To a solution of 3-[(10S)-4-(2-hydroxyphenyl)-1,5,6,8,12-pentazatricyclo[8.4.0.0^{2,7}]tetradeca-2,4,6-trien-12-yl]propanoic acid (70 mg, 197 umol, Intermediate QL) and methyl 2-piperazin-1-ylspiro[3.3]heptane-6-carboxylate (70.4 mg, 295 umol, Intermediate QP) in DMSO (2 mL) was added EDCI (56.6 mg, 295 umol), HOAt (40.2 mg, 295 umol) and DIEA (127 mg, 985 umol). The mixture was then stirred at 25° C. for 12 hrs. On completion, the reaction mixture was purified directly by reversed-phase HPLC (0.1% FA condition) to give the title compound (120 mg, 98% yield) as a white solid. LC-MS (ESI⁺) m/z 576.4 (M+H)⁺.

Step 2-(S)-6-(4-(3-(2-(2-hydroxyphenyl)-6a,7,9,10-tetrahydro-5H-pyrazino[1′,2′:4,5]pyrazino[2,3-c]pyridazin-8(6H)-yl)propanoyl)piperazin-1-yl)spiro[3.3]heptane-2-carboxylic acid. To a solution of methyl 2-[4-[3-[(10S)-4-(2-hydroxyphenyl)-1,5,6,8,12-pentazatricyclo[8.4.0.0^{2,7}]tetradeca-2,4,6-trien-12-yl]propanoyl]piperazin-1-yl]spiro[3.3]heptane-6-carboxylate (120 mg, 208 umol) in THF (1 mL) was added LiOH·H₂O (2 M, 0.5 mL). The mixture was stirred at 25° C. for 1 hr. On completion, the reaction mixture was filtered and concentrated under reduced pressure to give a residue. The crude product was purified by reversed-phase HPLC (0.1% HCl condition) to give the title compound (30 mg, 24% yield) as a white solid. LC-MS (ESI⁺) m/z 562.4 (M+H)⁺.

### Tert-butyl 6-(2-fluoropyrimidin-5-yl)-2-azaspiro[3.3]heptane-2-carboxylate (Intermediate QS)

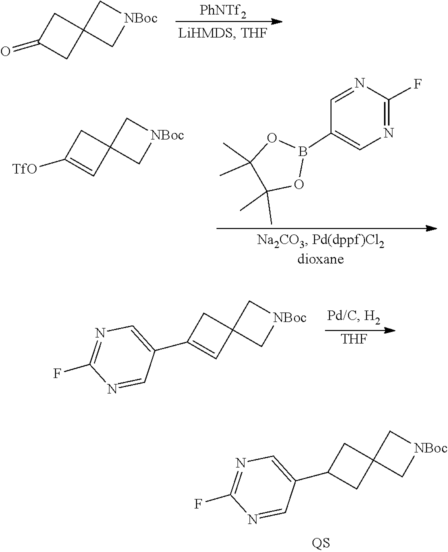

Step 1-Tert-butyl 6-(((trifluoromethyl)sulfonyl)oxy)-2-azaspiro[3.3]hept-5-ene-2-carboxylate. To a solution of tert-butyl 6-oxo-2-azaspiro[3.3]heptane-2-carboxylate (3 g, 14.2 mmol) in THF (30 mL) was added LiHMDS (1 M, 28.4 mL) at −78° C. The mixture was then stirred at −78° C. for 1 h. Then 1,1,1-trifluoro-N-phenyl-N-(trifluoromethylsulfonyl)methanesulfonamide (7.61 g, 21.3 mmol) was added in THF (45 mL) at −78° C. Then the mixture was stirred at 25° C. for 12 h. On completion, the reaction mixture was quenched with NH4C₁ (30 mL) at 0° C., and then diluted with EA 30 mL and extracted with EA (60 mL×3). The combined organic layers were washed with NaCl (60 mL×3), dried over Na₂SO₄, filtered and concentrated under reduced pressure to give a residue. The residue was purified by column chromatography (SiO₂, Petroleum ether/Ethyl acetate=100/1 to 30/1) to give the title compound (3.9 g, 80% yield,) was obtained as a yellow oil. ¹H NMR (400 MHz, CHLOROFORM-d) δ ppm 1.45 (s, 9H) 3.07 (s, 2H) 4.07-4.16 (m, 4H) 5.54 (s, 1H) 7.29-7.36 (m, 5H) 7.38-7.45 (m, 4H).

Step 2-Tert-butyl 6-(2-fluoropyrimidin-5-yl)-2-azaspiro[3.3]hept-5-ene-2-carboxylate. To a solution of tert-butyl 6-(trifluoromethylsulfonyloxy)-2-azaspiro[3.3]hept-6-ene-2-carboxylate (1.5 g, 4.37 mmol) and 2-fluoro-5-(4,4,5,5-tetramethyl-1,3,2-dioxaborolan-2-yl)pyrimidine (1.47 g, 6.55 mmol, CAS #1352796-65-6) in dioxane (25 mL) and H₂O (5 mL) was added Pd(dppf)Cl₂ (319 mg, 436 umol) and Na₂CO₃ (1.39 g, 13.1 mmol). The mixture was then stirred at 80° C. for 4 hrs. On completion, the reaction mixture was filtered and quenched with H₂O (50 mL) at 25° C., and then diluted with EA (50 mL) and extracted with EA (50 mL×3). The combined organic layers were washed with NaCl (50 mL×3), dried over Na₂SO₄, filtered and concentrated under reduced pressure to give a residue. The residue was purified by column chromatography (SiO₂, Petroleum ether/Ethyl acetate=50/1 to 5/1) to give the title compound (0.9 g, 69% yield) as a yellow solid. LC-MS (ESI⁺) m/z 236.2 (M−56+1)⁺.

Step 3-Tert-butyl 6-(2-fluoropyrimidin-5-yl)-2-azaspiro[3.3]heptane-2-carboxylate. To a solution of tert-butyl 6-(2-fluoropyrimidin-5-yl)-2-azaspiro[3.3]hept-6-ene-2-carboxylate (0.9 g, 3.09 mmol) in THF (20 mL) was added Pd/C (10 wt %, 450 mg) and Pd(OH)₂/C (20 wt %, 450 mg) under N₂ atmosphere. The suspension was degassed and purged with H₂ 3 times. The mixture was then stirred under H₂ (45 Psi) at 25° C. for 12 hrs. On completion, the reaction mixture was filtered with a kieselguhr and concentrated under reduced pressure to give the title compound (0.8 g) as a yellow solid. LC-MS (ESI⁺) m z 237.9 (M+1)⁺.

### (S)-2-(8-(1-(5-(2-azaspiro[3.3]heptan-6-yl)pyrimidin-2-yl)piperidin-4-yl)-6,6a,7,8,9,10-hexahydro-5H-pyrazino[1′,2′:4,5]pyrazino[2,3-c]pyridazin-2-yl)phenol (Intermediate QT)

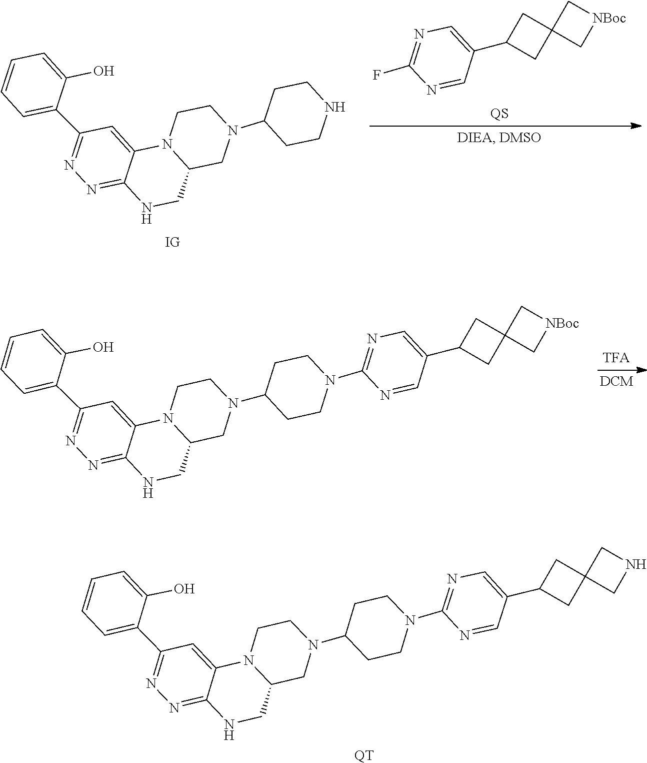

Step 1-(S)-tert-butyl 6-(2-(4-(2-(2-hydroxyphenyl)-6a,7,9,10-tetrahydro-5H-pyrazino[1′,2′:4,5]pyrazino[2,3-c]pyridazin-8(6H)-yl)piperidin-1-yl)pyrimidin-5-yl)-2-azaspiro[3.3]heptane-2-carboxylat. To a solution of 2-[(1OS)-12-(4-piperidyl)-1,5,6,8,12-pentazatricyclo[8.4.0.02,7]tetradeca-2,4,6-trien-4-yl]phenol (200 mg, 327 umol, AcOH, Intermediate IG) in DMSO (3 mL) was added DIEA (211 mg, 1.64 mmol) and tert-butyl 6-(2-fluoropyrimidin-5-yl)-2-azaspiro[3.3]heptane-2-carboxylate (192 mg, 655 umol, Intermediate QS). The mixture was then stirred at 60° C. for 12 hrs. On completion, the crude product was purified by reversed-phase HPLC (0.1% FA condition). After prep. HPLC purification, the eluent was concentrated to remove organic solvents. The residual aqueous solution was lyophilized to give the title compound (150 mg, 65% yield, FA) as a yellow solid. LC-MS (ESI⁺) m/z 640.5 (M+H)⁺.

Step 2-(S)-2-(8-(1-(5-(2-azaspiro[3.3]heptan-6-yl)pyrimidin-2-yl)piperidin-4-yl)-6,6a,7,8,9,10-hexahydro-5H-pyrazino[1′,2′:4,5]pyrazino[2,3-c]pyridazin-2-yl)phenol. To a solution of tert-butyl 6-[2-[4-[(10S)-4-(2-hydroxyphenyl)-1,5,6,8,12-pentazatricyclo[8.4.0.02,7]tetradeca-2,4,6-trien-12-yl]-1-piperidyl]pyrimidin-5-yl]-2-azaspiro[3.3]heptane-2-carboxylate (150 mg, 234 umol) in DCM (1.5 mL) was added TFA (462 mg, 4.05 mmol). The mixture was then stirred at 25° C. for 1 hr. On completion, the reaction mixture was concentrated under reduced pressure to give a residue. The crude product was purified by reversed-phase HPLC (0.1% FA condition). After prep. HPLC purification, the eluent was concentrated to remove organic solvents. The residual aqueous solution was lyophilized to give the title compound (100 mg, 72% yield, FA) as a yellow oil. LC-MS (ESI⁺) m/z 540.4 (M+H)⁺.

### (S)-4-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)benzoic acid (Intermediate QU)

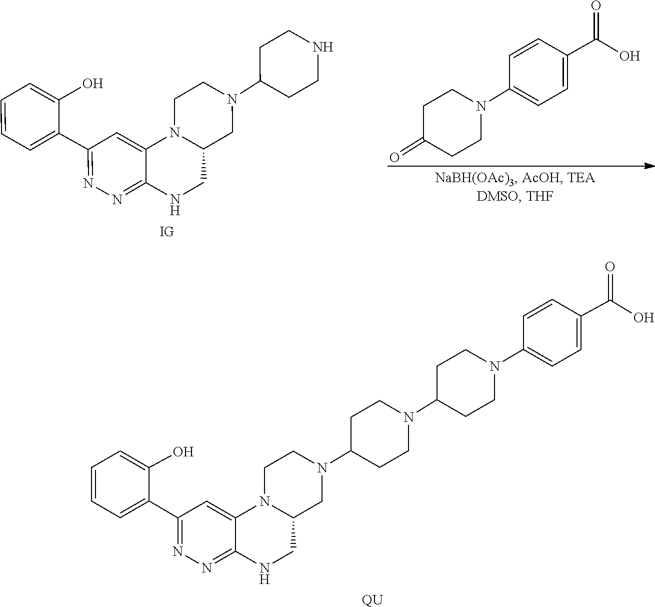

To a solution of 2-[(1OS)-12-(4-piperidyl)-1,5,6,8,12-pentazatricyclo[8.4.0.02,7]tetradeca-2,4,6-trien-4-yl]phenol (50 mg, 124 umol, HCl, Intermediate IG) in THF (1 mL) and DMSO (0.5 mL) was added TEA (25.1 mg, 248 umol, 34.5 uL), 4-(4-oxo-1-piperidyl)benzoic acid (81.6 mg, 372 umol, CAS #197446-34-7) and AcOH (22.4 mg, 372 umol, 21.29 uL). The mixture was stirred at 25° C. for 2 hrs and then NaBH(OAc)₃ (78.9 mg, 372 umol) was added at 0° C. The mixture was stirred at 25° C. for 10 hrs. On completion, the crude product was purified by reversed-phase HPLC(0.1% FA condition) to give the title compound (15 mg, 18% yield, FA salt) as a white solid. LC-MS (ESI⁺) m/z 570.4 (M+H)⁺.

### 2-(Aminomethyl)-3-chloro-5-ethynylphenol (Intermediate RC)

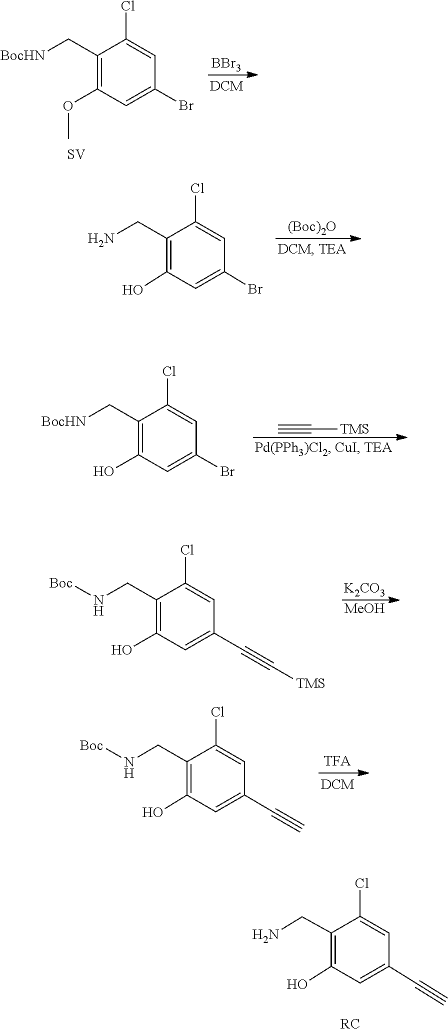

Step 1-2-(Aminomethyl)-5-bromo-3-chlorophenol. To a solution of tert-butyl 4-bromo-2-chloro-6-methoxybenzylcarbamate (500 mg, 1.43 mmol, Intermediate SV) in DCM (8 mL) was added BBr₃ (6.30 g, 4.28 mmol, 17% solution) at 0° C., then the reaction was stirred at 25° C. for 12 hrs. On completion, the reaction mixture was quenched with NaHCO₃ (aq,10 mL) and extracted by dichloromethane (3×5 mL). The extracts were washed by brine (10 mL) and dried over anhydrous sodium sulfate, filtered and concentrated in vacuo to give the title compound (500 mg) as yellow solid. ¹H NMR (400 MHz, CHLOROFORM-d) 6=7.35 (d, J=1.2 Hz, 1H), 7.19-7.14 (m, 1H), 6.95 (s, 1H), 5.44-5.36 (m, 1H), 4.52 (s, 2H).

Step 2-Tert-butyl 4-bromo-2-chloro-6-hydroxybenzylcarbamate. To a solution of 2-(aminomethyl)-5-bromo-3-chlorophenol (50 mg, 211 umol) in DCM (2 mL) was added TEA (42.8 mg, 42 umol) and (Boc)₂O (92.3 mg, 423 umol) at 25° C., then the reaction was stirred at 25° C. for 2 hrs. On completion, the reaction mixture was concentrated in vacuo to give the crude residue. The residue was purified by column chromatography (SiO₂, Petroleum ether/Ethyl acetate=10/1 to 5/1) to give the title compound (60 mg, 85% yield) as yellow oil. ¹H NMR (400 MHz, CHLOROFORM-d) δ=7.38 (br d, J=8.0 Hz, 3H), 4.60 (s, 2H), 1.60-1.56 (m, 9H).

Step 3-Tert-butyl 2-chloro-6-hydroxy-4-((trimethylsilyl)ethynyl)benzylcarbamate. To a solution of tert-butyl 4-bromo-2-chloro-6-hydroxybenzylcarbamate (200 mg, 594 umol) in TEA (10 mL) was added CuI (22.6 mg, 119 umol), Pd(PPh₃)₂Cl₂ (41.7 mg, 59.4 umol) and ethynyl(trimethyl)silane (875 mg, 8.91 mmol) in a 30 mL of sealed tube at 25° C., then the reaction was stirred at 100° C. for 12 hrs. On completion, the reaction mixture was filtered and concentrated in vacuo to give the crude residue. The crude product was purified by reversed-phase HPLC (0.10% FA condition) to give the title compound (160 mg, 67% yield, FA) as colorless oil. LC-MS (ESI⁺) m/z 298.1. (M+H−56)⁺.

Step 4-Tert-butyl 2-chloro-4-ethynyl-6-hydroxybenzylcarbamate. To a solution of tert-butyl 2-chloro-6-hydroxy-4-((trimethylsilyl)ethynyl)benzylcarbamate (160 mg, 452 umol) in MeOH (5 mL) was added K₂CO₃ (62.5 mg, 452 umol) at 25° C., then the reaction was stirred at 25° C. for 2 hrs. On completion, the reaction mixture was concentrated in vacuo to give the title compound (108 mg) as yellow solid. LC-MS (ESI⁺) m/z 282.1 (M+H)⁺.

Step 5-2-(Aminomethyl)-3-chloro-5-ethynylphenol. To a solution of tert-butyl tert-butyl 2-chloro-4-ethynyl-6-hydroxybenzylcarbamate (108 mg, 383 umol) in DCM (1 mL) was added TFA (665 mg, 5.83 mmol) at 25° C., then the reaction was stirred at 25° C. for 1 hr. On completion, the reaction mixture was concentrated in vacuo to give the crude residue. The crude product was purified by reversed-phase HPLC (0.1% FA condition) to give the title compound (40 mg, 58% yield, FA) as white solid. LC-MS (ESI⁺) m/z 165.1. (M+H−17)⁺.

### (S)-2-(1-Aminoethyl)-5-ethynylphenol (Intermediate RD)

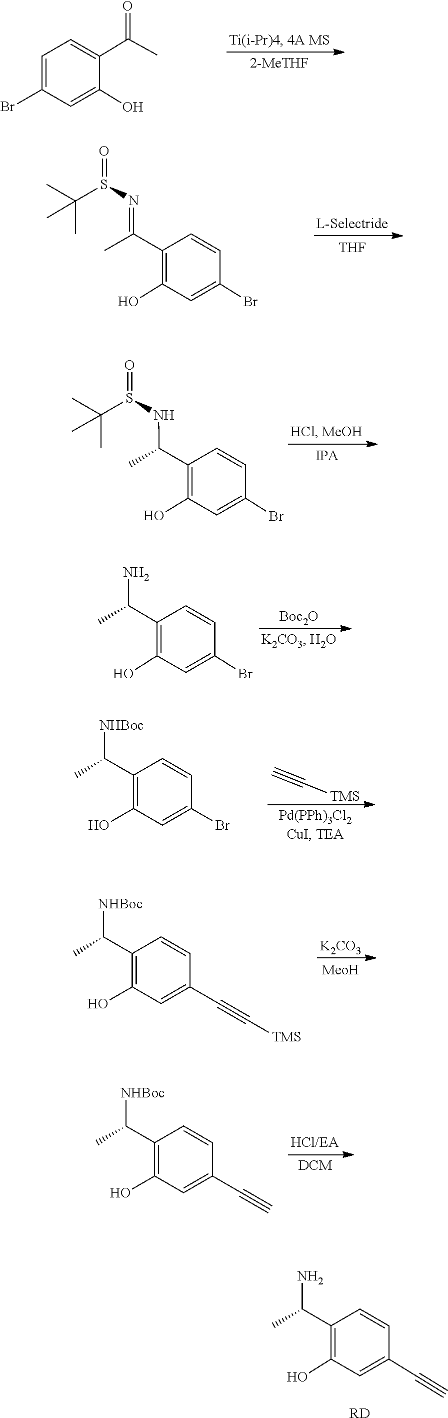

Step 1-(R,E)-N-(1-(4-bromo-2-hydroxyphenyl)ethylidene)-2-methylpropane-2-sulfinamide. To a solution of 1-(4-bromo-2-hydroxy-phenyl)ethanone (10 g, 46.5 mmol) and (R)-2-methylpropane-2-sulfinamide (16.9 g, 139 mmol) in 2,5-dimethylfuran (10 mL) was added Ti(i-PrO)4 (39.6 g, 139 mmol, 41.2 mL) and 4 Å molecular sieves (500 mg) at 0° C. The mixture was then stirred at 80° C. for 2 hr. On completion, the reaction mixture was filtered to give the solution and then diluted with H₂O (10 mL) and extracted with EA (10 mL×3). The combined organic layers were washed with NaCl (10 mL×1), dried over Na₂SO₄, filtered and concentrated under reduced pressure to give a residue. The mixture was purified by MPLC (SiO₂, PE:EA=10:1 to 5:1) to give the title compound (7 g, 40% yield) as a white solid. LC-MS (ESI⁺) m/z 319.9 (M+H)⁺.

Step 2-(R)—N—((S)-1-(4-bromo-2-hydroxyphenyl)ethyl)-2-methylpropane-2-sulfinamide. A mixture of (NE,R)—N-[1-(4-bromo-2-hydroxy-phenyl)ethylidene]-2-methyl-propane-2-sulfinamide (5.7 g, 17.9 mmol) in THF (50 mL) was degassed and purged with N₂ 3 times. Then L-selectride (1 M, 53.7 mL) was added at 0° C., and then the mixture was stirred at 0-25° C. for 3 hr under N₂ atmosphere. On completion, the mixture was quenched with H₂O (50 mL at 0° C., and then extracted by EA (100 mL×3). The combined organic layer was washed with NaCl (100 mL×3) and dried over Na₂SO₄, then the mixture was concentrated under reduce pressure to give a residue. The residue was purified by column chromatography (SiO₂, Petroleum ether/Ethyl acetate=20/1 to 3/1) to give the title compound (1 g, 17% yield) as a yellow solid. LC-MS (ESI⁺) m/z 322.0 (M+H+2)⁺.

Step 3-(S)-2-(1-aminoethyl)-5-bromophenol. To a solution of (R)—N-[(1S)-1-(4-bromo-2-hydroxy-phenyl)ethyl]-2-methyl-propane-2-sulfinamide (0.5 g, 1.56 mmol) in MeOH (4 mL) was added HCl (12 M, 1.03 mL) in IPA (1 mL).The mixture was then stirred at 25° C. for 1 hr. On completion, the mixture was concentrated under reduce pressure to give the title compound (0.35 g). LC-MS (ESI⁺) m/z 200.8 (M−NH₂)⁺

Step 4-(S)-tert-butyl (1-(4-bromo-2-hydroxyphenyl)ethyl)carbamate. To a solution of 2-[(1S)-1-aminoethyl]-5-bromo-phenol (0.35 g, 1.62 mmol) in H₂O (3 mL) was added K₂CO₃ (1.34 g, 9.72 mmol) and Boc₂O (393 mg, 1.80 mmol, 414 uL). The mixture was stirred at 25° C. for 12 hr. On completion, the mixture was quenched with H₂O (50 mL) at 0° C., and then extracted with EA (100 mL×3). The combined organic layer was washed with NaCl (100 mL×3), dried over Na₂SO₄, then the mixture was concentrated under reduce pressure to give the title compound (330 mg) as a white solid. LC-MS (ESI⁺) m/z 199.0(M−Boc−NH₂)⁺.

Step 5-(S)-tert-butyl (1-(2-hydroxy-4-((trimethylsilyl)ethynyl)phenyl)ethyl)carbamate. A mixture of tert-butyl N-[(1S)-1-(4-bromo-2-hydroxy-phenyl)ethyl]carbamate (330 mg, 1.04 mmol) ethynyl(trimethyl)silane (615 mg, 6.26 mmol, 868 uL), CuI (19.9 mg, 104 umol), and Pd(PPh₃)₂Cl₂ (36.6 mg, 52.2 umol) in TEA (8 mL) was degassed and purged with N₂ three times. Then the mixture was stirred at 70° C. for 12 hr under N₂ atmosphere. On completion, the reaction mixture was diluted with H₂O (20 mL) and extracted with EA (20 mL×3). The combined organic layers were washed with NaCl (10 mL×3), dried over Na₂SO₄, filtered and concentrated under reduced pressure to give a residue. The residue was purified by column chromatography (SiO₂, Petroleum ether/Ethyl acetate=40/1 to 5/1) to give the title compound (150 mg, 43% yield) as a white solid. LC-MS (ESI⁺) m/z 356.1(M+23)⁺.

Step 6-(S)-tert-butyl (1-(2-hydroxy-4-((trimethylsilyl)ethynyl)phenyl)ethyl)carbamate. To a solution of tert-butyl N-[(1S)-1-[2-hydroxy-4-(2-trimethylsilylethynyl)phenyl]ethyl]carbamate (150 mg, 450 umol) in MeOH (2 mL) was added K₂CO₃ (124 mg, 899 umol). The mixture was then stirred at 25° C. for 1 hr. On completion, the reaction mixture was diluted with EA (150 mL) and extracted with H₂O (50 mL×3). The combined organic layer was washed with NaCl (40 mL×30), dried over Na₂SO₄, filtered and concentrated under reduced pressure to give a residue. The residue was purified by column chromatography (SiO₂, Petroleum ether/Ethyl acetate=100/1 to 10/1) to give the title compound (100 mg, 83% yield) as a white solid. LC-MS (ESI⁺) m/z 284.2(M+23)⁺.

Step 7-(S)-2-(1-aminoethyl)-5-ethynylphenol. To a solution of tert-butyl N-[(1S)-1-(4-ethynyl-2-hydroxy-phenyl)ethyl]carbamate (30 mg, 100 umol) in DCM (1 mL) was added HCl/dioxane (4 M, 0.2 mL). The mixture was then stirred at 25° C. for 1 hr. On completion, the reaction mixture was concentrated under reduced pressure to give the title compound (30 mg) as a white solid. LC-MS (ESI⁺) m/z 145.3(M−NH₂)⁺.

### (2S, 4R)-4-hydroxy-N—((S)-4-phenylbut-3-yn-2-yl) pyrrolidine-2-carboxamide (Intermediate RE)

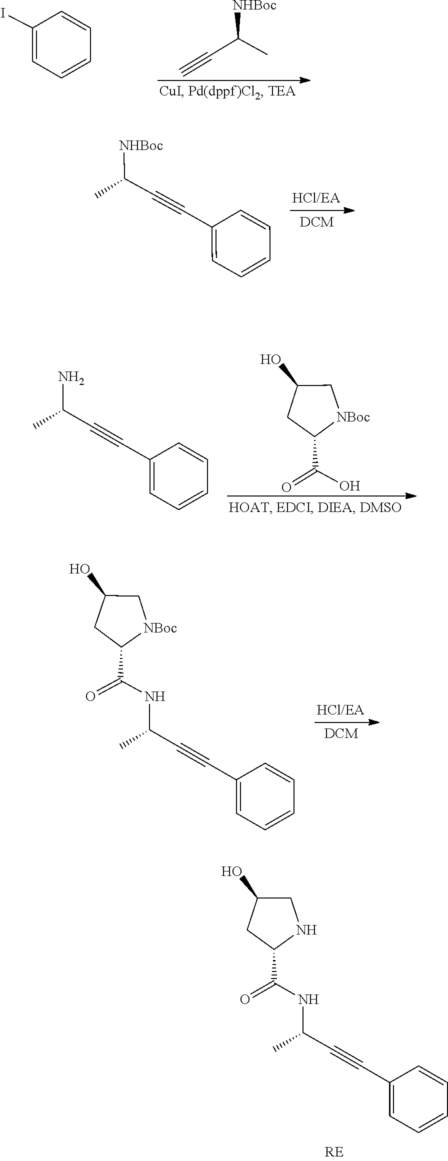

Step 1-(S)-tert-butyl (4-phenylbut-3-yn-2-yl) carbamate. To a solution of iodobenzene (1.65 g, 8.07 mmol, 899 uL, CAS #591-50-4) in TEA (20 mL) was added Pd(PPh₃)₂Cl₂ (377 mg, 538 umol), CuI (51.2 mg, 269 umol) and tert-butyl N-[(1S)-1-methylprop-2-ynyl]carbamate (910 mg, 5.38 mmol, CAS #118080-79-8). The mixture was stirred at 40° C. for 6 hrs. On completion, the reaction mixture was diluted with H₂O (30 mL) and extracted with DCM (30 mL×3). The combined organic layers were washed with aqueous NaCl (30 mL×3), dried over Na₂SO₄, filtered and concentrated under reduced pressure to give a residue. The residue was purified by column chromatography (SiO₂, Petroleum ether/Ethyl acetate=50/1 to 40/1) to give the title compound (1.3 g, 98% yield) as a white oil. LC-MS (ESI⁺) m/z 190.0 (M+H−C₄H₈)⁺.

Step 2-(S)-4-phenylbut-3-yn-2-amine. To a solution of tert-butyl N-[(1S)-1-methyl-3-phenyl-prop-2-ynyl] carbamate (1.2 g, 4.9 mmol) in DCM (10 mL) was added HCl/EtOAc (4 M, 1.22 mL). The mixture was stirred at 25° C. for 4 hrs. On completion, the reaction mixture was concentrated under reduced pressure to give the title compound (704 mg, HCl) as a white solid. LC-MS (ESI⁺) m/z 129.3 (M+H−NH₃)⁺.

Step 3-(2S,4R)-tert-butyl 4-hydroxy-2-(((S)-4-phenylbut-3-yn-2-yl)carbamoyl)pyrrolidine-1-carboxylate. To a solution of (2S)-4-phenylbut-3-yn-2-amine (704 mg, 4.85 mmol) in DMSO (20 mL) was added DIEA (3.13 g, 24.2 mmol, 4.22 mL), HOAt (990 mg, 7.27 mmol, 1.02 mL), EDCI (1.39 g, 7.27 mmol), and (2S,4R)-1-tert-butoxycarbonyl-4-hydroxy-pyrrolidine-2-carboxylic acid (1.35 g, 5.82 mmol, CAS #13726-69-7). The mixture was then stirred at 25° C. for 4 hrs. On completion, the reaction mixture was diluted with H₂O (60 mL) and extracted with EA (30 mL×3). The combined organic layers were washed with aqueous NaCl (30 mL×3), dried over Na₂SO₄, filtered and concentrated under reduced pressure to give a residue. The residue was purified by column chromatography (SiO₂, Petroleum ether/Ethyl acetate=8/1 to 1/1) to give the title compound (640 mg, 36% yield) as a white solid. LC-MS (ESI⁺) m/z 258.8 (M+H−Boc)⁺.

Step 4-(2S, 4R)-4-hydroxy-N—((S)-4-phenylbut-3-yn-2-yl) pyrrolidine-2-carboxamide. A solution of tert-butyl (2S, 4R)-4-hydroxy-2-[[(1S)-1-methyl-3-phenyl-prop-2-ynyl] carbamoyl]pyrrolidine-1-carboxylate (160 mg, 446 umol) in HCl/EtOAc (0.2 mL) and DCM (1 mL) was stirred at 25° C. for 2 hrs. On completion, the reaction mixture was concentrated under reduced pressure to give the title compound (160 mg, HCl) as a red solid. LC-MS (ESI⁺) m/z 259.1 (M+H)⁺.

### Ethyl 3-methyl-2-(3-(2-oxoethoxy) isoxazol-5-yl)butanoate (Intermediate RF)

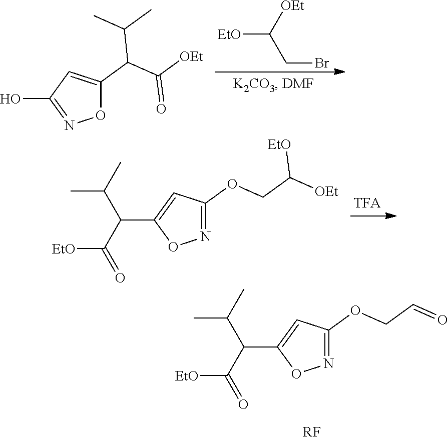 [SMILES: CCOC(=O)C(c1cc(O)no1)C(C)C ; CCOC(CBr)OCC ; CCOC(=O)C(c1cc(OCC(OCC)OCC)no1)C(C)C ; CCOC(=O)C(c1cc(OCC=O)no1)C(C)C ; "RF"]

Step 1-Ethyl 2-(3-(2,2-diethoxyethoxy)isoxazol-5-yl)-3-methylbutanoate. To a solution of ethyl 2-(3-hydroxyisoxazol-5-yl)-3-methyl-butanoate (2 g, 9.38 mmol, synthesized via Steps 1-4 of Intermediate IH) in DMF (50 mL) was added K₂CO₃ (5.19 g, 37.5 mmol), and 2-bromo-1,1-diethoxy-ethane (2.77 g, 14.1 mmol, 2.12 mL, CAS #2032-35-1). The mixture was stirred at 70° C. for 16 hrs. On completion, the reaction mixture was quenched with sat. NH₄Cl (100 mL) at 25° C., and then diluted with ethyl acetate (100 mL) and extracted with ethyl acetate (100 mL×3). The combined organic layers were washed with sat. NaCl (100 mL×1), dried over Na₂SO₄, filtered and concentrated under reduced pressure to give a residue The mixture was purified by MPLC (SiO₂, PE:EA=10:1 to 5:1). to give the title compound (2.1 g, 61% yield) as a white solid. 1H NMR (400 MHz, DMSO-d6) δ 5.86 (s, 1H), 4.77 (t, J=5.2 Hz, 1H), 4.21-4.05 (m, 4H), 3.75-3.60 (m, 2H), 3.59-3.45 (m, 2H), 3.39 (d, J=8.8 Hz, 1H), 2.27 (q, J=6.8 Hz, 1H), 1.26-1.07 (m, 9H), 0.93 (d, J=6.8 Hz, 3H), 0.85 (d, J=6.8 Hz, 3H).

Step 2-Ethyl 3-methyl-2-(3-(2-oxoethoxy) isoxazol-5-yl)butanoate. A solution of ethyl 2-[3-(2,2-diethoxyethoxy)isoxazol-5-yl]-3-methyl-butanoate (1 g, 3.04 mmol) in TFA (20 mL) was stirred at 60° C. for 3 hrs. On completion, the reaction mixture was diluted with extracted with EA (30 mL×3). The combined organic layers were washed with aqueous NaCl (30 mL×3), dried over Na₂SO₄, filtered and concentrated under reduced pressure to give the title compound (700 mg, TFA) as a yellow oil. LC-MS (ESI⁺) m/z 256.1 (M+H)⁺.

### 2-(3-(2-(4-(((S)-2-(2-hydroxyphenyl)-6a,7,9,10-tetrahydro-5H-pyrazino[1′,2′:4,5]pyrazino[2,3-c]pyridazin-8(6H)-yl)methyl)-[1,4′:1′,4″-terpiperidin]-1″-yl)ethoxy)isoxazol-5-yl)-3-methylbutanoic acid (Intermediate RG)

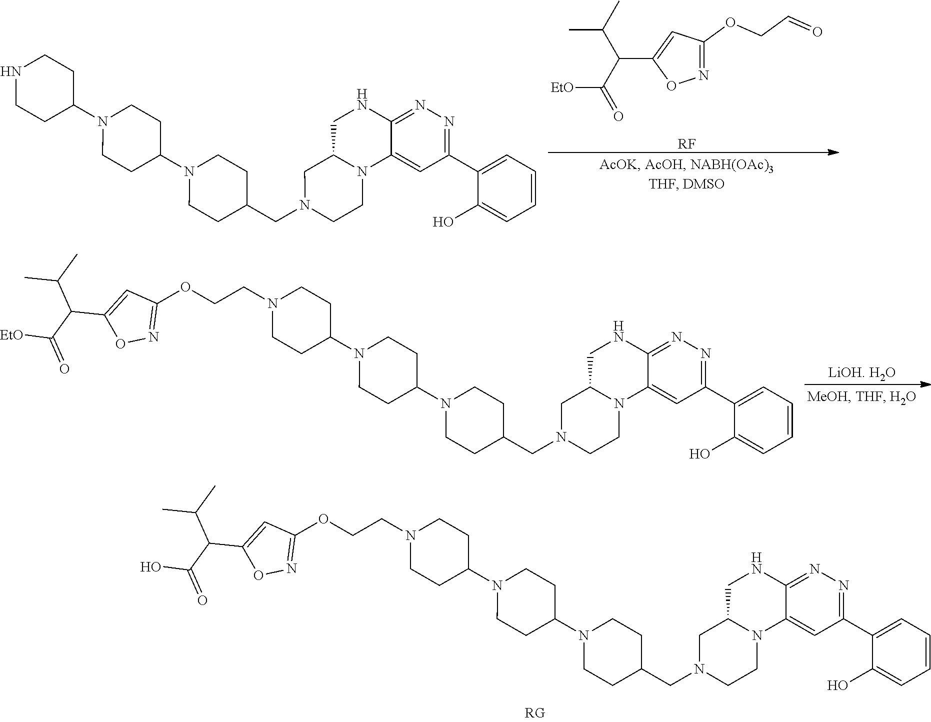

Step 1-Ethyl 2-(3-(2-(4-(((S)-2-(2-hydroxyphenyl)-6a,7,9,10-tetrahydro-5H-pyrazino[1′,2′:4,5]pyrazino[2,3-c]pyridazin-8(6H)-yl)methyl)-[1,4′: 1′,4″-terpiperidin]-1″-yl)ethoxy)isoxazol-5-yl)-3-methylbutanoate. To a solution of 2-[(1OS)-12-[[1-[1-(4-piperidyl)-4-piperidyl]-4-piperidyl]methyl]-1,5,6,8,12-pentazatricyclo[8.4.0.02,7]tetradeca-2,4,6-trien-4-yl]phenol (1.45 g, 2.65 mmol, Intermediate SX) in DMSO (6 mL) and THF (30 mL) was added AcOK (1.30 g, 13.3 mmol) at 25° C. for 0.5 hrs. Then AcOH (478 mg, 7.96 mmol, 455 uL) and ethyl 3-methyl-2-[3-(2-oxoethoxy) isoxazol-5-yl] butanoate (812 mg, 3.18 mmol, Intermediate RF) was added and the mixture was stirred at 25° C. for 1.5 hrs. Finally, to the solution was added NaBH(OAc)₃ (1.69 g, 7.96 mmol) at 0° C. Then the mixture was stirred at 25° C. for 4 hrs. On completion, the reaction mixture was quenched with H₂O (40 mL), and then diluted with H₂O (40 mL) and extracted with EA (40 mL×3). The combined organic layers were washed with aqueous NaCl (40 mL×3), dried over Na₂SO₄, filtered and concentrated under reduced pressure to give a residue. The crude product was purified by reversed-phase HPLC (FA) to give the title compound (970 mg, 45% yield) as a white solid. LC-MS (ESI⁺) m/z 786.4 (M+H)⁺.

Step 2-2-(3-(2-(4-(((S)-2-(2-hydroxyphenyl)-6a,7,9,10-tetrahydro-5H-pyrazino[1′,2′:4,5]pyrazino[2,3-c]pyridazin-8(6H)-yl)methyl)-[1,4′:1′,4″-terpiperidin]-1″-yl)ethoxy)isoxazol-5-yl)-3-methylbutanoic. To a solution of ethyl 2-[3-[2-[4-[4-[4-[[(10S)-4-(2-hydroxyphenyl)-1,5,6,8,12-pentazatricyclo[8.4.0.02,7]tetradeca-2,4,6-trien-12-yl]methyl]-1-piperidyl]-1-piperidyl]-1-piperidyl]ethoxy]isoxazol-5-yl]-3-methyl-butanoate (200 mg, 254 umol) in H₂O (0.5 mL), MeOH (0.5 mL), and THF (0.5 mL) was added LiOH·H₂O (85.4 mg, 2.04 mmol). The mixture was then stirred at 25° C. for 1 hr. On completion, the reaction mixture was concentrated under reduced pressure. The crude product was purified by reversed-phase HPLC (HCl) to give the title compound (97 mg, 47% yield) as a white solid. LC-MS (ESI⁺) m/z 758.5 (M+H)⁺.

### (S)-8-(5-iodopyrimidin-2-yl)-2-(2-(methoxymethoxy)phenyl)-6,6a,7,8,9,10-hexahydro-5H-pyrazino[1′,2′:4,5]pyrazino[2,3-c]pyridazine (Intermediate RH)

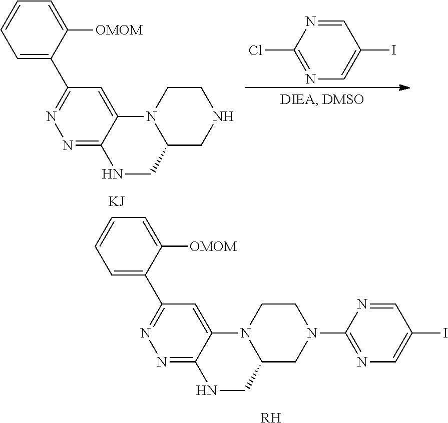

To a solution of (R)-2-(2-(methoxymethoxy)phenyl)-6,6a,7,8,9,10-hexahydro-5H-pyrazino[1′,2′:4,5]pyrazino[2,3-c]pyridazine (3 g, 9.16 mmol, Intermediate KJ) and 2-chloro-5-iodopyrimidine (2.42 mg, 10.1 mmol, CAS #32779-38-7) in DMSO (60 mL) was added DIEA (5.92 g, 45.8 moml). The mixture was stirred at 80° C. for 12 hrs. On completion, the reaction mixture was diluted with H₂O (100 mL) and washed with H₂O (50 mL×4), filtered and concentrated under reduced pressure to give the title compound (4 g) as a yellow solid. LC-MS (ESI⁺) m/z 532.1(M+H)⁺.

### Tert-butyl 4-(7-azaspiro[3.5]nonan-2-yl)piperazine-1-carboxylate (Intermediate RI)

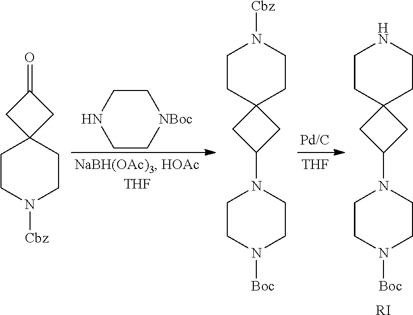

Step 1-Benzyl 2-(4-(tert-butoxycarbonyl)piperazin-1-yl)-7-azaspiro[3.5]nonane-7-carboxylate. To a solution of tert-butyl piperazine-1-carboxylate (500 mg, 2.68 mmol, CAS #143238-38-4) and benzyl 2-oxo-7-azaspiro[3.5]nonane-7-carboxylate (733.76 mg, 2.68 mmol, CAS #147610-98-8) in THF (10 mL) was added HOAc (483.62 mg, 8.05 mmol) and the mixture was stirred at 0° C. for 0.5 hrs. Then NaBH(OAc)₃ (1.71 g, 8.05 mmol) was added and the mixture was stirred at 0-20° C. for 11.5 hrs. On completion, the reaction mixture was quenched with aqueous NaHCO₃ (50 mL) at 0° C., and then diluted with H₂O (50 mL) and extracted with EA (50 mL×3). The combined organic layers were washed with aqueous NaCl (50 mL×2), dried over Na₂SO₄, filtered and concentrated under reduced pressure to give the title compound (1.1 g) as a yellow solid. ¹H NMR (400 MHz, CHLOROFORM-d) δ=7.32-7.20 (m, 5H), 5.04 (s, 2H), 3.41-3.33 (m, 6H), 3.32-3.27 (m, 2H), 2.60 (t, J=7.6 Hz, 1H), 2.17 (s, 4H), 1.93 (t, J=9.6 Hz, 2H), 1.61-1.42 (m, 6H), 1.38 (s, 9H).

Step 2-Tert-butyl 4-(7-azaspiro[3.5]nonan-2-yl)piperazine-1-carboxylate. To a solution of benzyl 2-(4-(tert-butoxycarbonyl)piperazin-1-yl)-7-azaspiro[3.5]nonane-7-carboxylate (500 mg, 1.13 mmol) in THF (10 mL) was added Pd/C (10 wt %, 300 mg) under N₂ atmosphere. The suspension was degassed under vacuum and purged with H₂ several times. The mixture was stirred under H₂ (15 psi) at 20° C. for 5 hrs. On completion, the reaction mixture was filtered and concentrated under reduced pressure to give the title compound (350 mg) as an off-white solid. LC-MS (ESI⁺) m/z 310.0(M+H)⁺.

### (S)-2-(8-(5-(2-(piperazin-1-yl)-7-azaspiro[3.5]nonan-7-yl)pyrimidin-2-yl)-6,6a,7,8,9,10-hexahydro-5H-pyrazino[1′,2′:4,5]pyrazino[2,3-c]pyridazin-2-yl)phenol (Intermediate RJ)

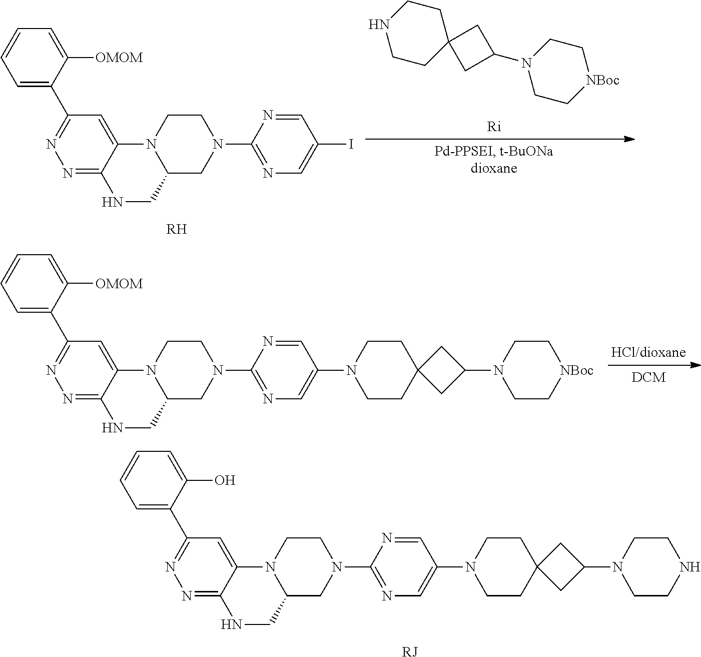

Step 1-(S)-tert-butyl 4-(7-(2-(2-(2-(methoxymethoxy)phenyl)-6a,7,9,10-tetrahydro-5H-pyrazino[1′,2′:4,5]pyrazino[2,3-c]pyridazin-8(6H)-yl)pyrimidin-5-yl)-7-azaspiro[3.5]nonan-2-yl)piperazine-1-carboxylate. To a solution of tert-butyl 4-(7-azaspiro[3.5]nonan-2-yl)piperazine-1-carboxylate (200 mg, 700 umol, Intermediate RI) and (S)-8-(5-iodopyrimidin-2-yl)-2-(2-(methoxymethoxy)phenyl)-6,6a,7,8,9,10-hexahydro-5H-pyrazino[1′,2′:4,5]pyrazino[2,3-c]pyridazine (264 mg, 497 umol, Intermediate RH) in dioxane (2 mL) was added t-BuONa (2 M, 1.49 mL) and Pd-PEPPSI-IHeptCl (48 mg, 49.7 umol). The mixture was stirred at 120° C. for 2 hrs under N₂ and microwave. On completion, the reaction mixture was quenched with aqueous NH₄Cl (10 mL) at 20° C., and then diluted with H₂O (10 mL) and extracted with DCM (20 mL×3). The combined organic layers were dried over Na₂SO₄, filtered and concentrated under reduced pressure to give a residue. The residue was purified by column chromatography (SiO₂, DCM/MeOH=10/0 to 10/1) to give the title compound (100 mg, 11% yield) as a yellow solid. LC-MS (ESI⁺) m/z 713.3(M+H)⁺.

Step 2-(S)-2-(8-(5-(2-(piperazin-1-yl)-7-azaspiro[3.5]nonan-7-yl)pyrimidin-2-yl)-6,6a,7,8,9,10-hexahydro-5H-pyrazino[1′,2′:4,5]pyrazino[2,3-c]pyridazin-2-yl)phenol. To a solution of (S)-tert-butyl 4-(7-(2-(2-(2-(methoxymethoxy)phenyl)-6a,7,9,10-tetrahydro-5H-pyrazino[1′,2′:4,5]pyrazino[2,3-c]pyridazin-8(6H)-yl)pyrimidin-5-yl)-7-azaspiro[3.5]nonan-2-yl)piperazine-1-carboxylate (100 mg, 140 umol) in DCM (2 mL) was added HCl/dioxane (4 M, 700 uL). The mixture was stirred at 20° C. for 10 min. On completion, the reaction mixture was concentrated under reduced pressure to give a residue. The residue was purified by reversed-phase HPLC (0.1% FA condition) to give the title compound (25 mg, 27% yield) as a yellow solid. LC-MS (ESI⁺) m/z 569.1(M+H)⁺.

### (R)-7-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperazin-1-yl)spiro[3.5]nonane-2-carboxylic acid (Intermediate RK)

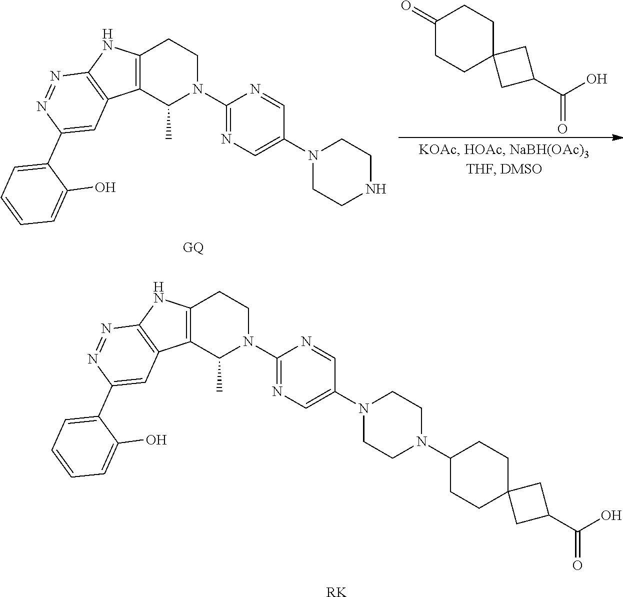 [SMILES: C[C@@H]1c2c([nH]c3nnc(-c4ccccc4O)cc23)CCN1c1ncc(N2CCNCC2)cn1 ; O=C1CCC2(CC1)CC(C(=O)O)C2 ; C[C@@H]1c2c([nH]c3nnc(-c4ccccc4O)cc23)CCN1c1ncc(N2CCN(C3CCC4(CC3)CC(C(=O)O)C4)CC2)cn1 ; "RK"]

To a solution of 2-[(3R)-3-methyl-4-(5-piperazin-1-ylpyrimidin-2-yl)-4,8,10,11-tetrazatricyclo[7.4.0.0^{2,7}]trideca-1(9),2(7),10,12-tetraen-12-yl]phenol (500 mg, 1.04 mmol, Intermediate GQ) in THF (7 mL) and DMSO (1 mL) was added KOAc (307 mg, 3.13 mmol) and the mixture was stirred at 25° C. for 0.5 hour. Next, HOAc (188 mg, 3.13 mmol) and 7-oxospiro[3.5]nonane-2-carboxylic acid (190 mg, 1.04 mmol, CAS #1440962-16-2) was added and the mixture was stirred at 25° C. for 0.5 hour. Finally, NaBH(OAc)₃ (553 mg, 2.61 mmol) was added and the mixture was stirred at 0-25° C. for 12 hrs. On completion, the reaction mixture was concentrated under reduced pressure to remove THF. The crude product was purified by reversed-phase HPLC (0.1% NH₃•H₂O) to give the title compound (200 mg, 31% yield) as a yellow solid. LC-MS (ESI⁺) m/z 609.4 (M+H)⁺.

### (5-Ethynyl-3-methoxy-2-pyridyl)methanamine (Intermediate RL)

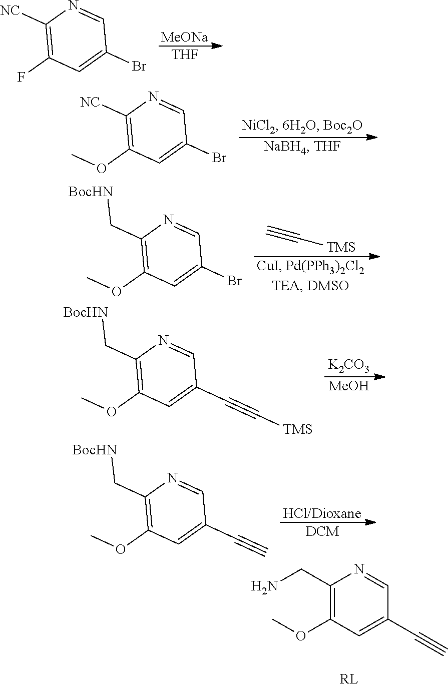

Step 1-5-Bromo-3-methoxypicolinonitrile. To a solution of 5-bromo-3-fluoro-pyridine-2-carbonitrile (20 g, 99.5 mmol, CAS #886373-28-0) in THF (150 mL) was added sodium methanolate (5.4 M, 20 mL). The mixture was then stirred at 70° C. for 16 hrs. On completion, the reaction was poured into HOAc (200 mL), then diluted with water (500 mL), and extracted with EtOAc (150 mL×3). The combined organic layer was washed with brine (150 mL), dried over Na₂SO₄, filtered and concentrated in vacuo. The crude was purified by silica gel column chromatography: (Petroleum ether: EtOAc from 100:1 to 10:1) to afford the title compound (11 g, 47% yield) as a white solid. ¹H NMR (400 MHz, DMSO-d₆) δ=8.46 (d, J=1.6 Hz, 1H), 8.17 (d, J=1.6 Hz, 1H), 4.00 (s, 3H).

Step 2-Tert-butyl ((5-bromo-3-methoxypyridin-2-yl)methyl)carbamate. 5-bromo-3-methoxy-pyridine-2-carbonitrile (10 g, 50 mmol) was added to BH₃.THF (1 M, 235 mL) at 0° C., then the mixture was stirred at 20° C. for 2 hrs. On completion, Boc₂O (20.5 g, 93.9 mmol, 22 mL) was added and the mixture was stirred at 20° C. for 1 hr. On completion, the reaction was diluted with water (500 mL), and extracted with EtOAc (200 mL×3). The combined organic layer was washed with brine (200 mL), dried over Na₂SO₄, filtered and concentrated in vacuo. The crude was purified by silica gel column chromatography: (Petroleum ether: EtOAc from 100:1 to 10:1) to afford the title compound (5.5 g, 49% yield) as a yellow solid. LC-MS (ESI⁺) m/z 317.0 (M+H)⁺.

Step 3-Tert-butyl ((3-methoxy-5-((trimethylsilyl)ethynyl)pyridin-2-yl)methyl)carbamate. To a solution of tert-butyl N-[(5-bromo-3-methoxy-2-pyridyl)methyl]carbamate (5.5 g, 17.3 mmol) and ethynyltrimethylsilane (17 g, 173.4 mmol) in DMSO (100 mL) were added Pd(PPh₃)₂Cl₂ (609 mg, 867 umol), TEA (5.26 g, 52 mmol) and CuI (330 mg, 1.73 mmol). The mixture was degassed and purged with N₂ thre times and then stirred at 60° C. for 2 hrs. On completion, the reaction was diluted with water (200 mL), and extracted with EtOAc (60 mL×3). The combined organic layer was washed with brine (100 mL), dried over Na₂SO₄, filtered and concentrated in vacuo. The crude was purified by silica gel column chromatography: (Petroleum ether: EtOAc from 100:1 to 10:1) to afford the title compound (5 g, 78% yield) as a yellow solid. LC-MS (ESI⁺) m/z 335.1 (M+H)⁺.

Step 4-Tert-butyl ((5-ethynyl-3-methoxypyridin-2-yl)methyl)carbamate. To a solution of tert-butyl N-[[3-methoxy-5-(2-trimethylsilylethynyl)-2-pyridyl]methyl]carbamate (4.5 g, 13.5 mmol) in MeOH (50 mL) was added K₂CO₃ (3.72 g, 26.9 mmol). The mixture was stirred at 20° C. for 1 hr. On completion, the reaction was concentrated in vacuo. The crude was purified by silica gel column chromatography: (Petroleum ether: EtOAc from 50:1 to 3:1) to afford the title compound (2.7 g, 69% yield) as a yellow solid. LC-MS (ESI⁺) m/z 263.1 (M+H)⁺.

Step 5-(5-Ethynyl-3-methoxypyridin-2-yl)methanamine. To a solution of tert-butyl N-[(5-ethynyl-3-methoxy-2-pyridyl)methyl]carbamate (0.2 g, 762.5 umol) in DCM (4 mL) was added HCl/dioxane (4 M, 1.02 mL) at 5° C. The mixture was then stirred at 20° C. for 2 hrs. On completion, the reaction was filtered to afford the title compound (0.13 g, HCl) as a white solid. LC-MS (ESI⁺) m/z 163.1 (M+H)⁺.

### (2S,4R)-methyl 1-((S)-2-amino-3,3-dimethylbutanoyl)-4-hydroxypyrrolidine-2-carboxylate (Intermediate RM)

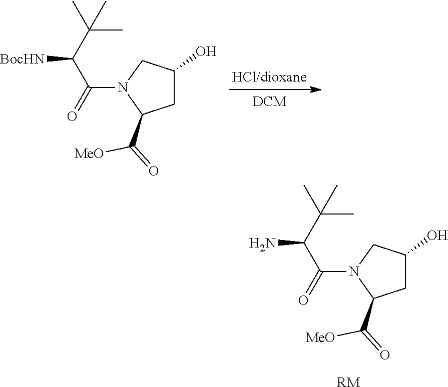

To a solution of methyl (2S,4R)-1-[2-(tert-butoxycarbonylamino)-3,3-dimethyl-butanoyl]-4-hydroxy-pyrrolidine-2-carboxylate (50 g, 139.50 mmol, CAS #630421-45-3) in DCM (400 mL) was added HCl/dioxane (10 M, 16.00 mL), then the mixture was stirred at 25° C. for 1 hr. On completion, the reaction mixture was concentrated under reduced pressure to give the title compound (40 g, HCl) as a yellow solid. LC-MS (ESI+) m/z 851.5 (M+H)⁺.

### (2S,4R)-4-hydroxy-1-((S)-2-(2-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxamido)-3,3-dimethylbutanoyl)pyrrolidine-2-carboxylic acid (Intermediate RN)

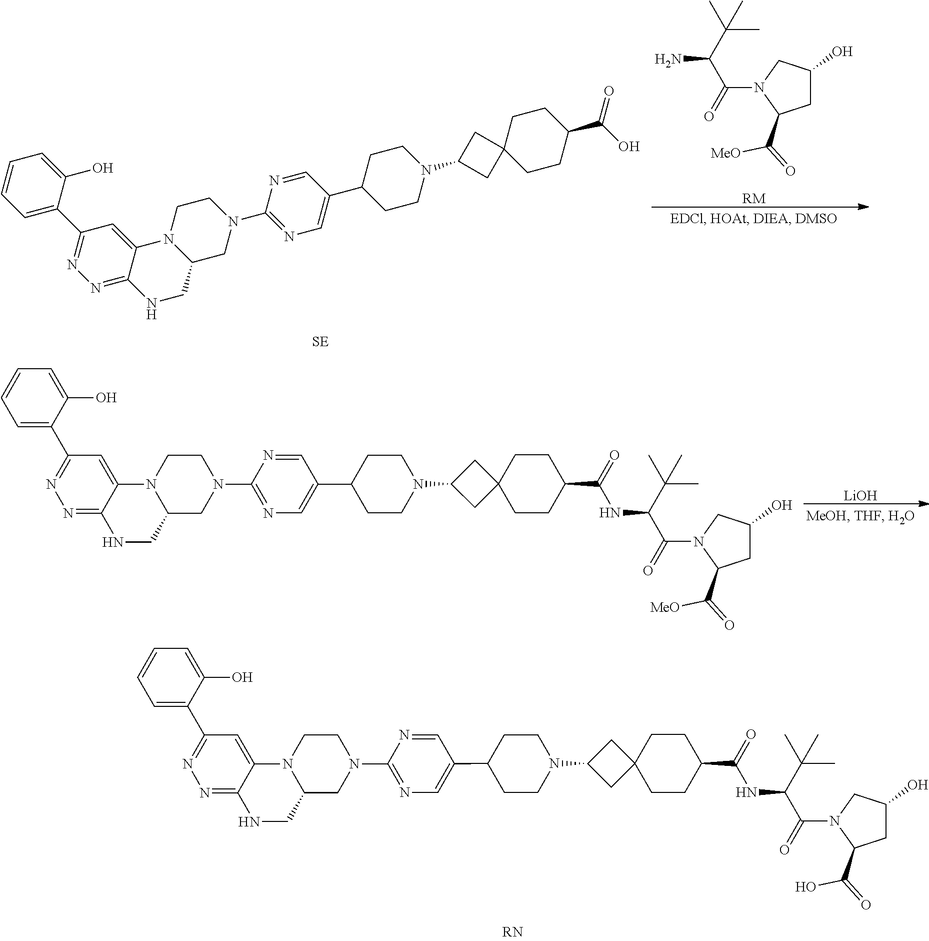

Step 1-(2S,4R)-methyl 4-hydroxy-1-((S)-2-(2-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxamido)-3,3-dimethylbutanoyl)pyrrolidine-2-carboxylate. To a solution of 2-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]spiro[3.5]nonane-7-carboxylic acid (250 mg, 386 umol, Intermediate SE) in DMSO (4 mL) was added EDCI (111 mg, 579 umol), HOAt (78.9 mg, 579 umol, 81.1 uL), DIEA (300 mg, 2.32 mmol, 404 uL) and methyl (2S,4R)-1-[(2S)-2-amino-3,3-dimethyl-butanoyl]-4-hydroxy-pyrrolidine-2-carboxylate (150 mg, 579 umol, Intermediate RM). Then the mixture was stirred at 25° C. for 12 hrs. The mixture was purified by reversed-phase flash (0.1% FA) to give the title compound (300 mg, FA salt) as a white solid. LC-MS (ESI⁺) m/z 851.5 (M+H)⁺.

Step 2-(2S,4R)-4-hydroxy-1-((S)-2-(2-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxamido)-3,3-dimethylbutanoyl)pyrrolidine-2-carboxylic acid. To a solution of methyl (2S,4R)-4-hydroxy-1-[(2S)-2-[[2-[4-[2-[(10S)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]spiro[3.5]nonane-7-carbonyl]amino]-3,3-dimethyl-butanoyl]pyrrolidine-2-carboxylate (300 mg, 353 umol) in MeOH (1.5 mL), H₂O (1.5 mL) and THF (1.5 mL) was added LiOH·H₂O (74.0 mg, 1.76 mmol). Then the mixture was stirred at 25° C. for 12 hrs. On completion, the mixture was purified by reversed-phase Flash (0.1% HCl) to give the title compound (80 mg, 24% yield) as a yellow solid. LC-MS (ESI⁺) m/z 837.9 (M+H)⁺.

### (2S,4R)-4-hydroxy-1-((S)-2-(2-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxamido)-3,3-dimethylbutanoyl)pyrrolidine-2-carboxlic acid (Intermediate RO)

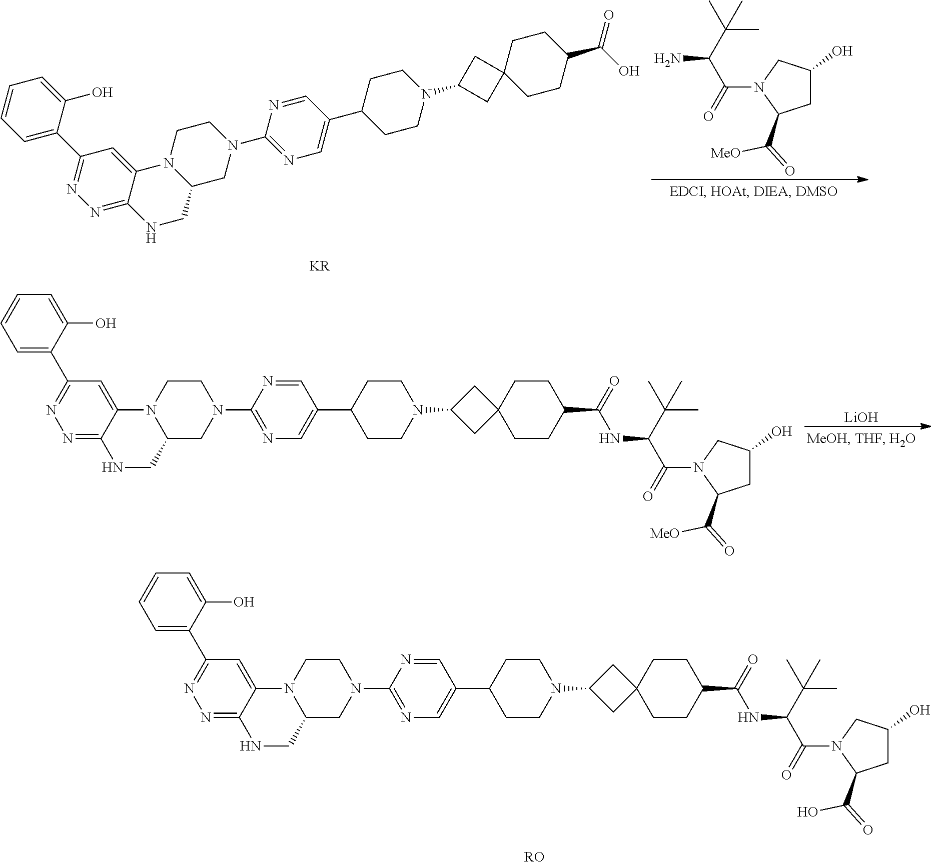

Step 1-(2S,4R)-methyl 4-hydroxy-1-((S)-2-(2-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxamido)-3,3-dimethylbutanoyl)pyrrolidine-2-carboxylate. To a solution of 2-[4-[2-[(1OS)-4-(2-hydroxyphenyl)-1,5,6,8,12-pentazatricyclo[8.4.0.02,7]tetradeca-2,4,6-trien-12-yl]pyrimidin-5-yl]-1-piperidyl]spiro[3.5]nonane-7-carboxylic acid (320 mg, 524 umol, Intermediate KR) in DMSO (7 mL) was added EDCI (151 mg, 786 umol), DIEA (339 mg, 2.62 mmol, 456 uL), and HOAt (107 mg, 786 umol). After 10 mins, methyl (2S,4R)-1-[(2S)-2-amino-3,3-dimethyl-butanoyl]-4-hydroxy-pyrrolidine-2-carboxylate (463 mg, 1.57 mmol, HCl, Intermediate RM) was added and the mixture was stirred at 25° C. for 12 hrs. On completion, the reaction mixture was purified by reversed-phase HPLC(0 0.1% FA condition) to give the title compound (130 mg, 28% yield, FA) as a yellow solid. LC-MS (ESI⁺) m/z 851.5 (M+H)⁺.

Step 2-(2S,4R)-4-hydroxy-1-((S)-2-(2-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxamido)-3,3-dimethylbutanoyl)pyrrolidine-2-carboxylic acid. To a solution of methyl (2S,4R)-4-hydroxy-1-[(2S)-2-[[2-[4-[2-[(1OS)-4-(2-hydroxyphenyl)-1,5,6,8,12-pentazatricyclo[8.4.0.02,7]tetradeca-2,4,6-trien-12-yl]pyrimidin-5-yl]-1-piperidyl]spiro[3.5]nonane-7-carbonyl]amino]-3,3-dimethyl-butanoyl]pyrrolidine-2-carboxylate (100 mg, 118 umol) in THF (0.3 mL), H₂O (0.3 mL) and MeOH (0.3 mL) was added LiOH·H₂O (24.6 mg, 587 umol). The mixture was stirred at 25° C. for 1 hr. On completion, the crude product was purified by reversed-phase HPLC(0.1% FA condition) to give the title compound (95 mg, 92% yield, FA) as a white solid. LC-MS (ESI⁺) m/z 837.2 (M+H)⁺.

### Phenyl ((S)-1-((2S,4R)-2-((4-ethynylbenzyl)carbamoyl)-4-hydroxypyrrolidin-1-yl)-5-hydroxy-3,3-dimethyl-1-oxopentan-2-yl)carbamate (Intermediate RP)

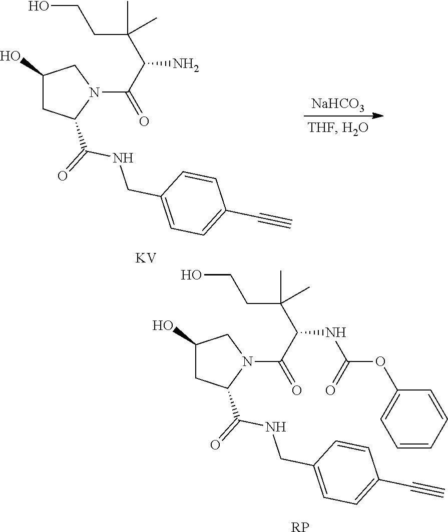

To a solution of (2S,4R)-1-[(2S)-2-amino-5-hydroxy-3,3-dimethyl-pentanoyl]-N-[(4-ethynylphenyl)methyl]-4-hydroxy-pyrrolidine-2-carboxamide (155 mg, 400.03 umol, Intermediate KV) in THF (2 mL) was added NaHCO₃ (134 mg, 1.60 mmol, 62.2 uL) in H₂O(0.1 mL) and phenyl carbonochloridate (75.2 mg, 480. umol, 60.13 ul, CAS #1885-14-9) in THF(0.1 mL) at 0° C. The mixture was then stirred at 0° C. for 1 hr. On completion, the reaction mixture was quenched diluted with H₂O (10 mL) and extracted with EA (20 mL×3). The combined organic layers were washed with brine (5 mL×3), dried over Na₂SO₄, filtered and concentrated under reduced pressure to give a residue. The residue was purified by column chromatography (SiO₂, Petroleum ether/Ethyl acetate=20/1 to 1/1) to give the title compound (70 mg, 30% yield) as a white solid. ¹H NMR (400 MHz, DMSO-d6) δ=8.62-8.53 (m, 1H), 7.91 (br d, J=9.3 Hz, 1H), 7.39-7.32 (m, 4H), 7.20 (br t, J=7.1 Hz, 1H), 7.14-7.05 (m, 2H), 5.76 (s, 1H), 5.13 (d, J=3.4 Hz, 1H), 4.46-4.38 (m, 2H), 4.34 (br d, J=4.3 Hz, 1H), 4.27-4.21 (m, 1H), 4.13 (s, 1H), 3.62 (br s, 1H), 3.49 (br d, J=5.5 Hz, 1H), 2.69 (s, 3H), 2.18 (s, 2H), 1.90 (s, 2H), 1.23 (br s, 2H), 1.07-0.95 (m, 6H); LC-MS (ESI⁺) m/z 352.5.

### Ethyl 3-methyl-2-(3-(4-oxopiperidin-1-yl)isoxazol-5-yl)butanoate (Intermediate RV)

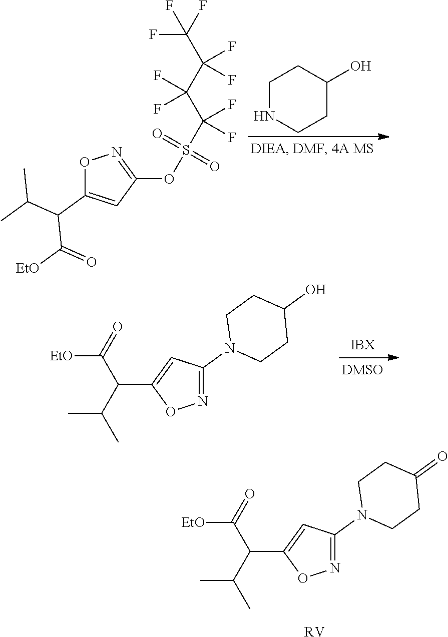

Step 1-Ethyl 2-(3-(4-hydroxypiperidin-1-yl)isoxazol-5-yl)-3-methylbutanoate

To a solution of ethyl 3-methyl-2-(3-(((perfluorobutyl)sulfonyl)oxy)isoxazol-5-yl)butanoate (2 g, 4.04 mmol, Intermediate IH) in DMF (20 mL) was added DIEA (3.13 g, 24.2 mmol), piperidin-4-ol (449 mg, 4.44 mmol, CAS #5382-16-1) and 4 Å molecular sieves (2 g). The mixture was stirred at 130° C. for 1 hr. On completion, the reaction mixture was concentrated in vacuo to give a residue. The crude product was purified by column chromatography (SiO2, Petroleum ether/Ethyl acetate=3/1 to 1/1) to give the title compound (600 mg, 49% yield) as a colorless oil. LC-MS (ESI⁺) m/z 297.5 (M+H)⁺.

Step 2-Ethyl 3-methyl-2-(3-(4-oxopiperidin-1-yl)isoxazol-5-yl)butanoate

To a mixture of ethyl 2-(3-(4-hydroxypiperidin-1-yl)isoxazol-5-yl)-3-methylbutanoate (500 mg, 1.69 mmol) in DMSO (5 mL) was added IBX (945 mg, 3.37 mmol). The mixture was then stirred at 25° C. for 0.5 hr. On completion, the reaction mixture was concentrated in vacuo to give a residue. The crude product was purified by column chromatography (SiO₂, Petroleum ether/Ethyl acetate=3/1 to 1/1) to give the title compound (300 mg, 60% yield) as a colorless oil. LC-MS (ESI⁺) m/z 295.1 (M+H)⁺.

### (S)-2-(8-([1,4′-bipiperidin]-4-ylmethyl)-6,6a,7,8,9,10-hexahydro-5H-pyrazino[1′,2′:4,5]pyrazino[2,3-c]pyridazin-2-yl)phenol (Intermediate RW)

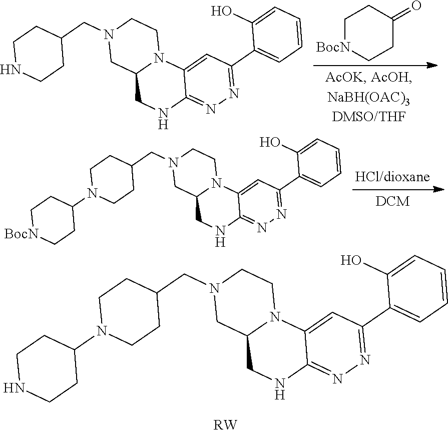 [SMILES: Oc1ccccc1-c1cc2c(nn1)NC[C@H]1CN(CC3CCNCC3)CCN21 ; CC(C)(C)OC(=O)N1CCC(=O)CC1 ; CC(C)(C)OC(=O)N1CCC(N2CCC(CN3CCN4c5cc(-c6ccccc6O)nnc5NC[C@H]4C3)CC2)CC1 ; Oc1ccccc1-c1cc2c(nn1)NC[C@H]1CN(CC3CCN(C4CCNCC4)CC3)CCN21 ; "RW"]

Step 1-(S)-tert-butyl 4-((2-(2-hydroxyphenyl)-6a,7,9,10-tetrahydro-5H-pyrazino[1′,2′:4,5]pyrazino[2,3-c]pyridazin-8(6H)-yl)methyl)-[1,4′-bipiperidine]-1′-carboxylate. To a solution of (S)-2-(8-(piperidin-4-ylmethyl)-6,6a,7,8,9,10-hexahydro-5H-pyrazino[1′,2′:4,5]pyrazino[2,3-c]pyridazin-2-yl)phenol (1.8 g, 4.73 mmol, Intermediate SW) in THF (10 mL) and DMSO (10 mL) was added AcOK (1.39 g, 14.2 mmol) at 25° C. and the mixture was stirred for 1 hr. Then tert-butyl 4-oxopiperidine-1-carboxylate (1.04 g, 5.20 mmol, CAS #79099-07-3) and AcOH (1.14 g, 18.9 mmol) was added and stirred for 2 hours. Last, NaBH(OAc)₃ (3.01 g, 14.2 mmol) was added at 0° C., then the mixture was stirred at 25° C. for 10 hrs. On completion, the reaction mixture was concentrated in vacuo to give a residue. The residue was purified by reversed-phase HPLC(0.1% FA condition) to give the title compound (1 g, 30% yield, FA) as a yellow solid. LC-MS (ESI⁺) m/z 564.4 (M+H)⁺.

Step 2-(S)-2-(8-([1,4′-bipiperidin]-4-ylmethyl)-6,6a,7,8,9,10-hexahydro-5H-pyrazino[1′,2′:4,5]pyrazino[2,3-c]pyridazin-2-yl)phenol. To a solution of (S)-tert-butyl 4-((2-(2-hydroxyphenyl)-6a,7,9,10-tetrahydro-5H-pyrazino[1′,2′:4,5]pyrazino[2,3-c]pyridazin-8(6H)-yl)methyl)-[1,4′-bipiperidine]-1′-carboxylate (1 g, 2 mmol) in DCM (2 mL) was added HCl/dioxane (4 M, 443 uL). The mixture was stirred at 25° C. for 1 hrs. On completion, the reaction mixture was concentrated in vacuo to give the title compound (800 mg, HCl) as a pink solid. LC-MS (ESI⁺) m/z 464.1 (M+H)⁺.

### Ethyl 2-(3-(4-(((S)-2-(2-hydroxyphenyl)-6a,7,9,10-tetrahydro-5H-pyrazino[1′,2′:4,5]pyrazino[2,3-c]pyridazin-8(6H)-yl)methyl)-[1,4′:1′,4″-terpiperidin]-1″-yl)isoxazol-5-yl)-3-methylbutanoate (Intermediate RX)

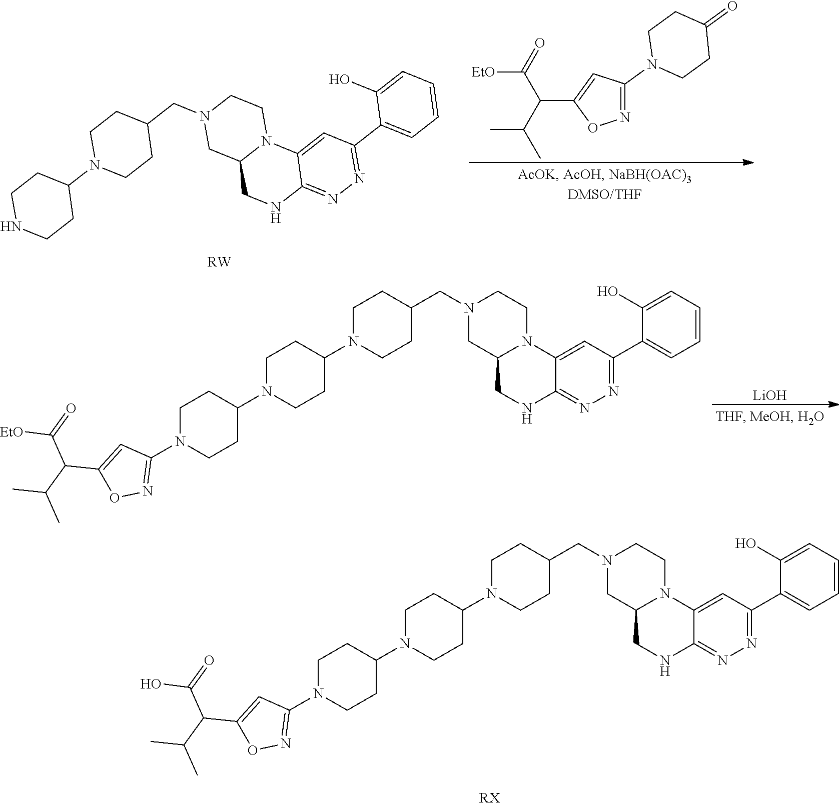

Step 1-Ethyl 2-(3-(4-(((S)-2-(2-hydroxyphenyl)-6a,7,9,10-tetrahydro-5H-pyrazino[1′,2′:4,5]pyrazino[2,3-c]pyridazin-8(6H)-yl)methyl)-[1,4′:1′,4″-terpiperidin]-1″-yl)isoxazol-5-yl)-3-methylbutanoate. To a solution of (S)-2-(8-([1,4′-bipiperidin]-4-ylmethyl)-6,6a,7,8,9,10-hexahydro-5H-pyrazino[1′,2′:4,5]pyrazino[2,3-c]pyridazin-2-yl)phenol (354 mg, 764 umol, Intermediate RW) in THF (0.5 mL) and DMSO (0.5 mL) was added AcOK (150 mg, 1.53 mmol) and the mixture was stirred for 1 hr. Then ethyl 3-methyl-2-(3-(4-oxopiperidin-1-yl)isoxazol-5-yl)butanoate (150 mg, 510 umol, Intermediate RV) and AcOH (122 mg, 2.04 mmol) was added and the mixture was stirred for 2 hours at 40° C. Lastly, NaBH(OAc)₃ (324 mg, 1.53 mmol) was added at 0° C. and then the mixture was stirred at 25° C. for 9 hrs. On completion, the reaction mixture was concentrated in vacuo to give a residue. The crude product was purified by reversed-phase HPLC (0.1% FA condition) to give the title compound (70 mg, 17% yield, FA) as a white solid. LC-MS (ESI⁺) m/z 742.5 (M+H)⁺.

Step 2-Ethyl 2-(3-(4-(((S)-2-(2-hydroxyphenyl)-6a,7,9,10-tetrahydro-5H-pyrazino[1′,2′:4,5]pyrazino[2,3-c]pyridazin-8(6H)-yl)methyl)-[1,4′: 1′,4″-terpiperidin]-1″-yl)isoxazol-5-yl)-3-methylbutanoate. To a solution of ethyl 2-(3-(4-(((S)-2-(2-hydroxyphenyl)-6a,7,9,10-tetrahydro-5H-pyrazino[1′,2′:4,5]pyrazino[2,3-c]pyridazin-8(6H)-yl)methyl)-[1,4′:1′,4″-terpiperidin]-1″-yl)isoxazol-5-yl)-3-methylbutanoate (100 mg, 135 umol) in H₂O (1 mL), THF (1 mL), and MeOH (1 mL) was added LiOH·H₂O (45.3 mg, 1.08 mmol). The mixture was stirred at 25° C. for 8 hrs. On completion, the reaction mixture was concentrated in vacuo to give a residue. The crude product was purified by reversed-phase HPLC (0.1% NH₃•H₂O) to give the title compound (80 mg, 75% yield) as a white solid. LC-MS (ESI⁺) m z 714.5 (M+H)⁺.

### (S)-5-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)picolinic acid (Intermediate RY)

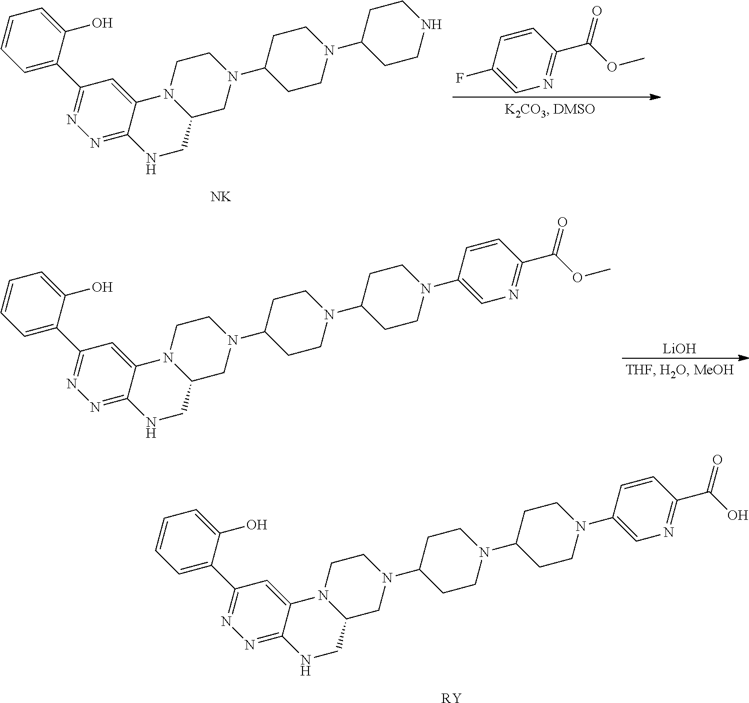

Step 1-(S)-methyl 5-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)picolinate. To a mixture of (S)-2-(8-([1,4′-bipiperidin]-4-yl)-6,6a,7,8,9,10-hexahydro-5H-pyrazino[1′,2′:4,5]pyrazino[2,3-c]pyridazin-2-yl)phenol (300 mg, 589 umol, HOAC, Intermediate NK) and methyl 5-fluoropicolinate (82.2 mg, 530 umol, CAS #107504-07-4) in DMSO (4 mL) was added K₂CO₃ (203 mg, 1.47 mmol). The mixture was then stirred at 60° C. for 12 hrs. On completion, the reaction mixture was filtered and concentrated under reduced pressure to give a residue. The residue was purified by prep-HPLC (NH₄HCO₃ condition) to give the title compound (65 mg, 92% purity, 17% yield) as a yellow solid. LC-MS (ESI+) m/z 585.4 (M+H)⁺.

Step 2-(S)-5-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)picolinic acid. To a mixture of (S)-methyl 5-(4-(2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)picolinate (65 mg, 111 umol) in H₂O (0.5 mL), MeOH (0.5 mL) and THF (0.5 mL) was added LiOH·H₂O (28 mg, 667 umol). The mixture was then stirred at 25° C. for 1 hr. On completion, the mixture was filtered and concentrated under reduced pressure to give a residue. The residue was purified by prep-HPLC (FA condition) to give the title compound (11 mg, 16% yield) as a yellow solid. LC-MS (ESI+) m/z 571.4 (M+H)⁺.

### Methyl 4-formylcyclohexanecarboxylate (Intermediate RZ)

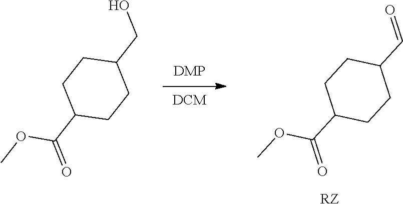

To a solution of methyl 4-(hydroxymethyl)cyclohexanecarboxylate (900 mg, 5.23 mmol CAS #110928-44-4) in DCM (10 mL) was added DMP (3.32 g, 7.84 mmol). The mixture was stirred at 0-20° C. for 2 hrs. On completion, the residue was diluted with H₂O (10 mL) and extracted with EA (10 mL×3). The combined organic layers were washed with aqueous NH4Cl (10 mL×3), dried over Na₂SO₄, filtered and concentrated under reduced pressure to give a residue. The residue was purified by column chromatography (SiO₂, Petroleum ether/Ethyl acetate=10/0 to 3/1) to give the title compound (650 mg, 73% yield) as a white solid.

### (S)-4-((4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)methyl)cyclohexanecarboxylic acid (Intermediate SA)

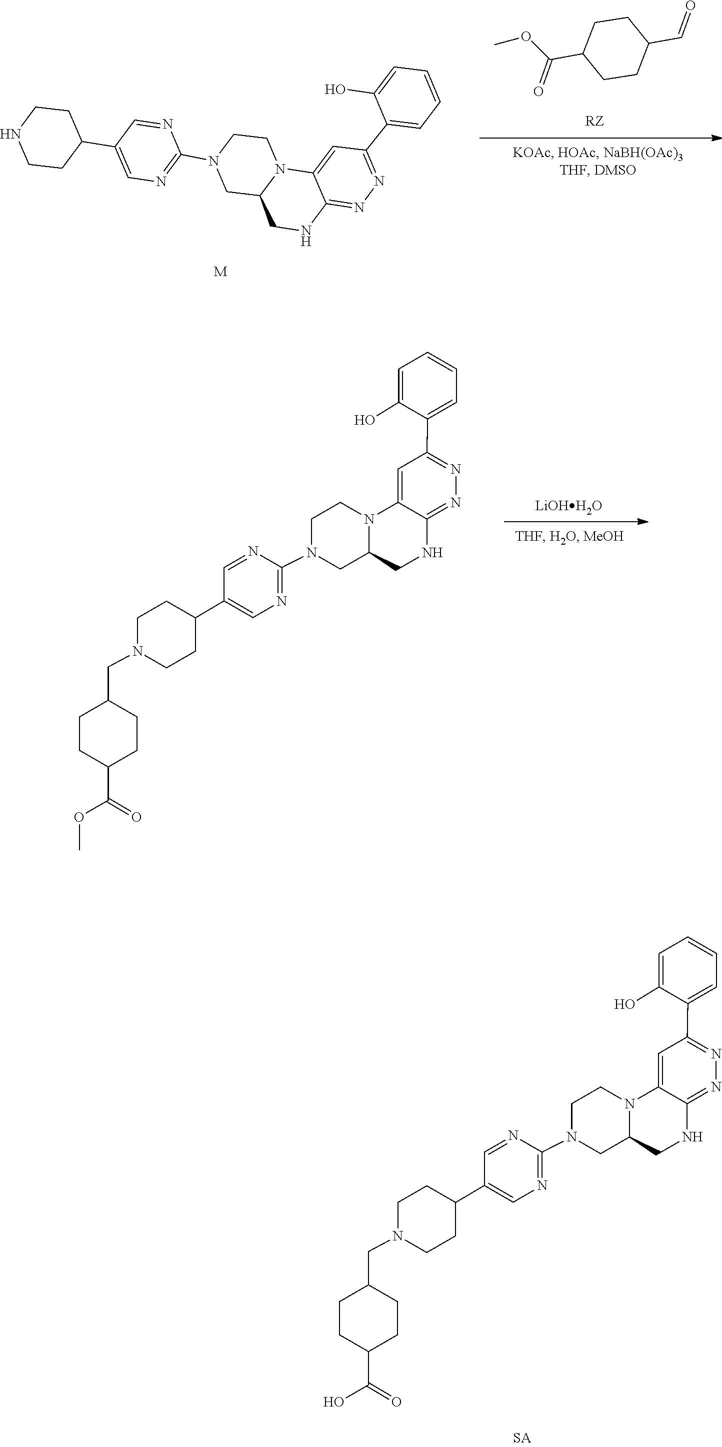

Step 1-(S)-methyl 4-((4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)methyl)cyclohexanecarboxylate. To a solution of methyl 4-formylcyclohexanecarboxylate (600 mg, 3.53 mmol, Intermediate RZ), 2-[(1OS)-12-[5-(4-piperidyl)pyrimidin-2-yl]-1,5,6,8,12-pentazatricyclo[8.4.0.0^{2,7}]tetradeca-2,4,6-trien-4-yl]phenol (1.88 g, 4.23 mmol, Intermediate M) in DMSO (8 mL) and THF (2 mL) was added HOAc (635.08 mg, 10.8 mmol) and KOAc (1.04 g, 10.8 mmol) and stirred at 0° C. for 0.5 hr. Then NaBH(OAc)₃ (2.24 g, 10.8 mmol) was added and the mixture was stirred at 0-20° C. for 1.5 hrs. On completion, the residue was diluted with H₂O (30 mL) and extracted with EA (30 mL×3). The combined organic layers were washed with aqueous NH₄Cl (30 mL×3), dried over Na₂SO₄, filtered and concentrated under reduced pressure to give a residue. The residue was purified by column chromatography (SiO₂, DCM/MeOH=10/0 to 10/1) to give the title compound (2.3 g, 96% yield) as a white solid. LC-MS (ESI⁺) m/z 599.5(M+H)⁺.

Step 2-(S)-4-((4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)methyl)cyclohexanecarboxylic acid. To a solution of methyl 4-[[4-[2-[(1OS)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]methyl]cyclohexanecarboxylate (1.2 g, 2.00 mmol) in THF (10 mL), MeOH (10 mL) and H₂O (10 mL) was added LiOH·H₂O (420 mg, 10.2 mmol). The mixture was then stirred at 25° C. for 2 hrs. On completion, the reaction mixture was quenched with 1M HCl until the pH=5, and then Na₂CO₃ aqueous solution was added until pH=8. The solid precipitation was then filtered and dried to give the title compound (370 mg, 30% yield) as a white solid. LC-MS (ESI⁺) m/z 585.0 (M+H)⁺.

### (2S,4R)—N-(4-ethynyl-2-fluorobenzyl)-4-hydroxypyrrolidine-2-carboxamide (Intermediate SC)

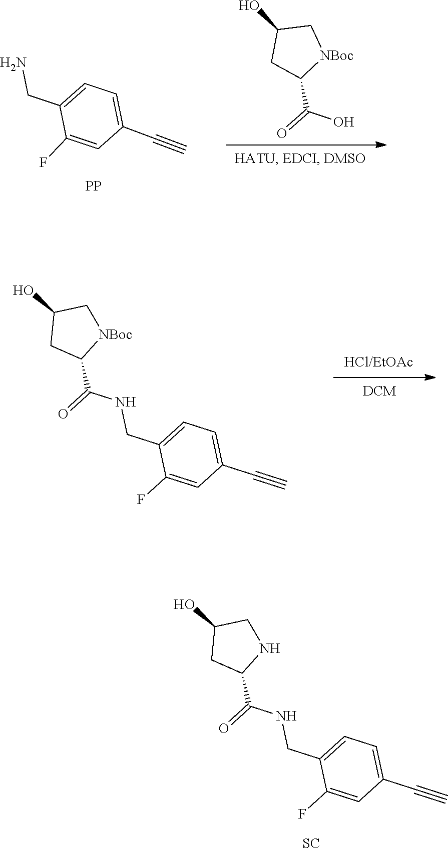

Step 1-(2S, 4R)-tert-butyl 2-((4-ethynyl-2-fluorobenzyl) carbamoyl)-4-hydroxypyrrolidine-1-carboxylate. To a solution of (4-ethynyl-2-fluoro-phenyl)methanamine (3.5 g, 23.5 mmol, Intermediate PP) in DMSO (20 mL) was added DIEA (12.1 g, 93.8 mmol, 16.3 mL), HATU (10.7 g, 28.2 mmol), and (2S,4R)-1-tert-butoxycarbonyl-4-hydroxy-pyrrolidine-2-carboxylic acid (6.5 g, 28.2 mmol, CAS #13726-69-7). The mixture was stirred at 25° C. for 2 hrs. On completion, the reaction mixture was concentrated under reduced pressure to give a residue. The crude product was purified by reversed-phase HPLC (0.1% FA condition) to give the title compound (3.5 g, 36% yield) as a white solid. LC-MS (ESI⁺) m/z 263.2 (M−99)⁺.

Step 2-(2S, 4R)—N-(4-ethynyl-2-fluorobenzyl)-4-hydroxypyrrolidine-2-carboxamide. To a solution of tert-butyl (2S,4R)-2-[(4-ethynyl-2-fluoro-phenyl)methylcarbamoyl]-4-hydroxy-pyrrolidine-1-carboxylate (500 mg, 1.38 mmol) in DCM (1.5 mL) was added HCl/EtOAc (4 M, 0.5 mL). The mixture was stirred at 25° C. for 2 hrs. On completion, the reaction mixture was concentrated under reduced pressure to give the title compound (500 mg, HCl) as a red solid. LC-MS (ESI⁺) m/z 263.0 (M+H)⁺.

### Phenyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate (Intermediate SD)

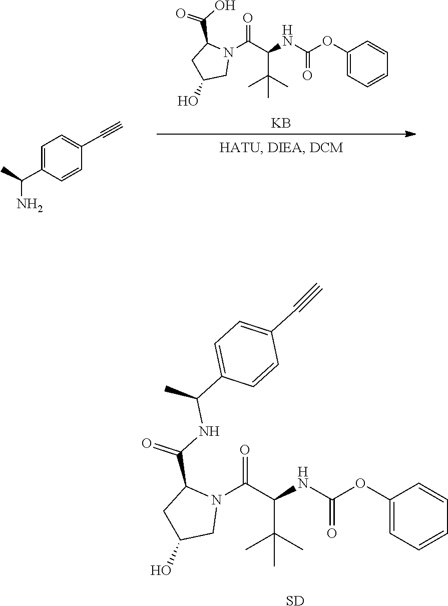

To a solution of (2S,4R)-1-[(2S)-3,3-dimethyl-2-(phenoxycarbonylamino)butanoyl]-4-hydroxy-pyrrolidine-2-carboxylic acid (12.0 g, 33.0 mmol, Intermediate KB) in DCM (100 mL) was added HATU (15.7 g, 41.3 mmol) and DIEA (21.3 g, 165 mmol) and (1S)-1-(4-ethynylphenyl)ethanamine (5 g, 27.5 mmol, Intermediate JC). The mixture was stirred at 25° C. for 12 hrs. On completion, the reaction mixture was concentrated under reduced pressure to remove DCM. The residue was diluted with H₂O (50 mL) and extracted with EA (50 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=10/1 to 1/3) give the title compound (10.6 g, 54% yield) as a yellow solid. LC-MS (ESI+) m/z 492.2 (M+H)⁺.

### (S)-2-(4-(2-(2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)spiro[3.5]nonane-7-carboxylic acid (Intermediate SE)

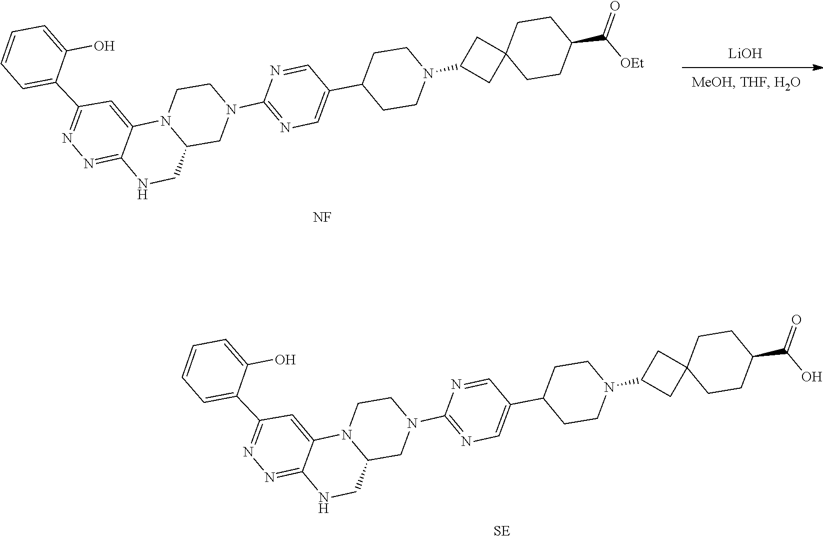

To a solution of ethyl 2-[4-[2-[(1OS)-4-(2-hydroxyphenyl)-1,5,6,8,12-pentazatricyclo[8.4.0.0^{2,7}]tetradeca-2,4,6-trien-12-yl]pyrimidin-5-yl]-1-piperidyl]spiro[3.5]nonane-7-carboxylate (2.5 g, 3.91 mmol, Intermediate NF) in MeOH (8 mL), THF (8 mL) and H₂O (8 mL) was added LiOH·H₂O (1.64 g, 39.1 mmol). The mixture was stirred at 25° C. for 12 hrs. On completion, the mixture was filtered and purified by reversed-phase Flash (0.1% HCl) to give the title compound (2.4 g, 98% purity, HCl salt) as a brown solid. LC-MS (ESI⁺) m/z 611.1 (M+H)⁺.

### (2S,4R)-tert-butyl 2-(((5-ethynyl-3-methoxypyridin-2-yl)methyl)carbamoyl)-4-hydroxypyrrolidine-1-carboxylate (Intermediate SF)

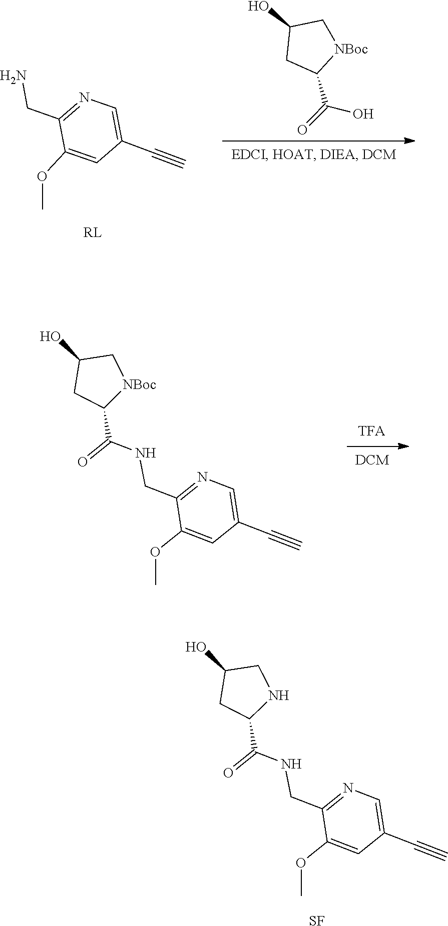

Step 1-(2S,4R)—N-((5-ethynyl-3-methoxypyridin-2-yl)methyl)-4-hydroxypyrrolidine-2-carboxamide. To a solution of (2S,4R)-1-(tert-butoxycarbonyl)-4-hydroxypyrrolidine-2-carboxylic acid (1.47 g, 6.36 mmol) in DCM (10 mL) was added EDCI (1.52 g, 7.95 mmol), HOAt (1.08 g, 7.95 mmol) and DIEA (3.43 g, 26.5 mmol, 4.62 mL) at 25° C. Then (5-ethynyl-3-methoxypyridin-2-yl)methanamine (860 mg, 5.30 mmol, Intermediate RL) was added at 25° C., then the reaction was stirred 25° C. for 1 hr. On completion, the reaction mixture was filtered and concentrated in vacuo to give the crude residue. The crude product was purified by reversed-phase HPLC (0.1% FA condition) to give the title compound (300 mg, 13% yield, FA) as white solid. LC-MS (ESI⁺) m/z 376.4. (M+H)⁺.

Step 2-(2S,4R)-tert-butyl 2-(((5-ethynyl-3-methoxypyridin-2-yl)methyl)carbamoyl)-4-hydroxypyrrolidine-1-carboxylate. To a solution of (2S,4R)—N-((5-ethynyl-3-methoxypyridin-2-yl)methyl)-4-hydroxypyrrolidine-2-carboxamide (100 mg, 266 umol) in DCM (1 mL) was added TFA (308 mg, 2.70 mmol) at 25° C. Then the reaction was stirred 25° C. for 1 hr. On completion, the reaction mixture was filtered and concentrated in vacuo to give the title compound (80 mg) as white solid. LC-MS (ESI⁺) m/z 276.0 (M−99)⁺.

### (S)-2-(2-(methoxymethoxy)phenyl)-8-(piperidin-4-yl)-6,6a,7,8,9,10-hexahydro-5H-pyrazino[1′,2′:4,5]pyrazino[2,3-c]pyridazine (Intermediate SH)

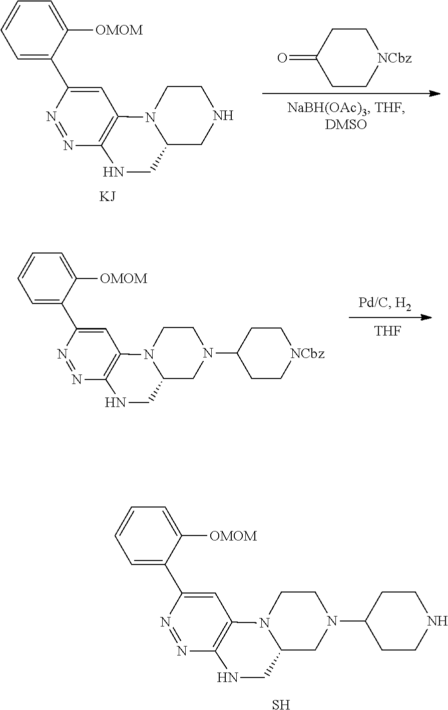

Step 1-(S)-benzyl 4-(2-(2-(methoxymethoxy)phenyl)-6a,7,9,10-tetrahydro-5H-pyrazino[1′,2′:4,5]pyrazino[2,3-c]pyridazin-8(6H)-yl)piperidine-1-carboxylate. To a solution of(10R)-4-[2-(methoxymethoxy)phenyl]-1,5,6,8,12-pentazatricyclo[8.4.0.02,7]tetradeca-2(7),3,5-triene (3 g, 9.16 mmol, Intermediate KJ) and benzyl 4-oxopiperidine-1-carboxylate (3.21 g, 13.75 mmol, 2.74 mL, CAS #19099-93-5) in THF (60 mL) was added 4 Å molecular sieves (6 g) and AcOH (550 mg, 9.16 mmol, 524 uL). The mixture was stirred at 25° C. for 2 hrs and then NaBH(OAc)₃ (3.88 g, 18.3 mmol) was added at 0° C. The mixture was then stirred at 25° C. for 12 hrs. The reaction mixture was quenched with aq. NaHCO₃ (50 mL) and extracted with EA (50 mL×3). The combined organic layers were washed with brine (20 mL), dried over Na₂SO₄, filtered and concentrated under reduced pressure to give a residue. The crude product was purified by reversed-phase HPLC(0.1% FA condition) to give title compound (5.7 g, 91% yield) as a yellow solid. LC-MS (ESI⁺) m/z 545.3 (M+H)⁺.

Step 2-(S)-2-(2-(methoxymethoxy)phenyl)-8-(piperidin-4-yl)-6,6a,7,8,9,10-hexahydro-5H-pyrazino[1′,2′:4,5]pyrazino[2,3-c]pyridazine. To a solution of benzyl 4-[(1OS)-4-[2-(methoxymethoxy)phenyl]-1,5,6,8,12-pentazatricyclo[8.4.0.02,7]tetradeca-2(7),3,5-trien-12-yl]piperidine-1-carboxylate (2.5 g, 3.7 mmol, FA) in THF (50 mL) was added Pd/C (2.5 g, 10 wt %). The mixture was stirred under H₂(45 psi) at 25° C. for 12 hrs. The reaction mixture was filtered with kieselguhr the filter cake was washed with MeOH (10 mL) and concentrated under reduced pressure to give a residue. The crude product was purified by reversed-phase HPLC(0.1% FA condition) to give title compound (1.5 g, 69% yield, FA) as a yellow solid. LC-MS (ESI⁺) m/z413.3 (M+H)⁺.

### Methyl 6-(2-fluoropyrimidin-5-yl)spiro[3.3]heptane-2-carboxylate (Intermediate SI)

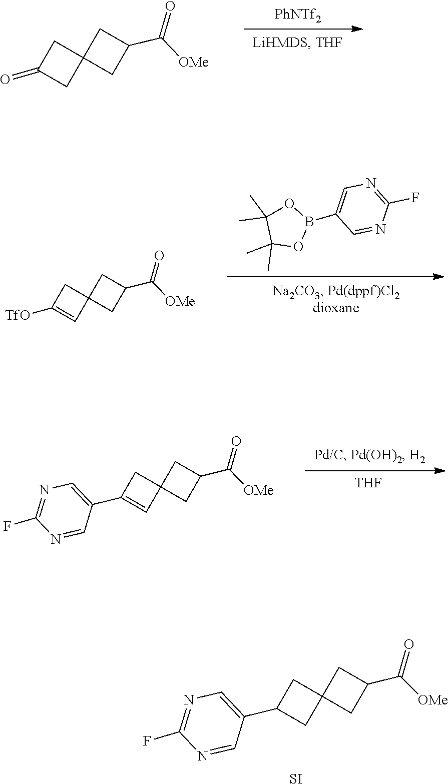

Step 1-Methyl 6-(((trifluoromethyl)sulfonyl)oxy)spiro[3.3]hept-5-ene-2-carboxylate. To a solution of methyl 2-oxospiro[3.3]heptane-6-carboxylate (1 g, 5.95 mmol, CAS #1138480-98-4) in THF (10 mL) was added LiHMDS (1 M, 7.43 mL) at −78° C. The mixture was stirred at −78 for 1 hr and then added 1,1,1-trifluoro-N-phenyl-N-(trifluoromethylsulfonyl)methanesulfonamide (3.19 g, 8.92 mmol, CAS #37595-74-7) in THF (10 mL) was added at −78° C. The mixture was then warmed to 25° C. and stirred at this temperature for 12 hrs. On completion, the reaction mixture was quenched with NH₄Cl (10 mL) at 0° C., and then diluted with EA (10 mL) and H₂O (5 mL), then extracted with EA (10 mL×3). The combined organic layers were washed with brine (30 mL), dried over anhydrous Na₂SO₄, filtered and concentrated under reduced pressure to give the title compound (1.2 g) as a yellow oil.

Step 2-Methyl 6-(2-fluoropyrimidin-5-yl)spiro[3.3]hept-5-ene-2-carboxylate. To a solution of methyl 2-(trifluoromethylsulfonyloxy)spiro[3.3]hept-2-ene-6-carboxylate (1.1 g, 3.66 mmol) and 2-fluoro-5-(4,4,5,5-tetramethyl-1,3,2-dioxaborolan-2-yl)pyrimidine (820 mg, 3.66 mmol, CAS #1352796-65-6) in dioxane (60 mL) and H₂O (10 mL) was added Pd(dppf)Cl₂ (268 mg, 366 umol) and Na₂CO₃ (1.16 g, 10.9 mmol). The mixture was then stirred at 80° C. for 4 hrs. On completion, the reaction mixture was quenched with NH₄Cl (20 mL) and extracted with EA (50 mL×3). The combined organic layers were washed with brine (30 mL), dried over anhydrous Na₂SO₄, filtered and concentrated under reduced pressure to give a residue. The residue was purified by column chromatography (SiO₂, Petroleum ether/Ethyl acetate=10/1 to 2/1) to give title compound (0.8 g, 82% yield) as a yellow oil. LC-MS (ESI⁺) m/z249 (M+H)⁺.

Step 3-Methyl 6-(2-fluoropyrimidin-5-yl)spiro[3.3]heptane-2-carboxylate. To a solution of methyl 2-(2-fluoropyrimidin-5-yl)spiro[3.3]hept-2-ene-6-carboxylate (0.8 g, 3.22 mmol) in THF (20 mL) was added Pd/C (0.4 g, 10 wt %) and Pd(OH)₂ (0.4 g, 20 wt %) under N₂ atmosphere. The suspension was degassed and purged with H₂ three times. The mixture was then stirred under H₂ (45 Psi) at 25° C. for 12 hrs. On completion, the reaction mixture was filtered with kieselguhr, then the filter cake was washed with MeOH (10 mL), then the filtrate was concentrated under reduced pressure to give the title compound (0.7 g) as colorless oil. LC-MS (ESI⁺) m/z251 (M+H)⁺.

### (S)-methyl 6-(2-(4-(2-(2-(methoxymethoxy)phenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylate (Intermediate SJ) and (S)-methyl 6-(2-(4-(2-(2-(methoxymethoxy)phenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylate (Intermediate SK)

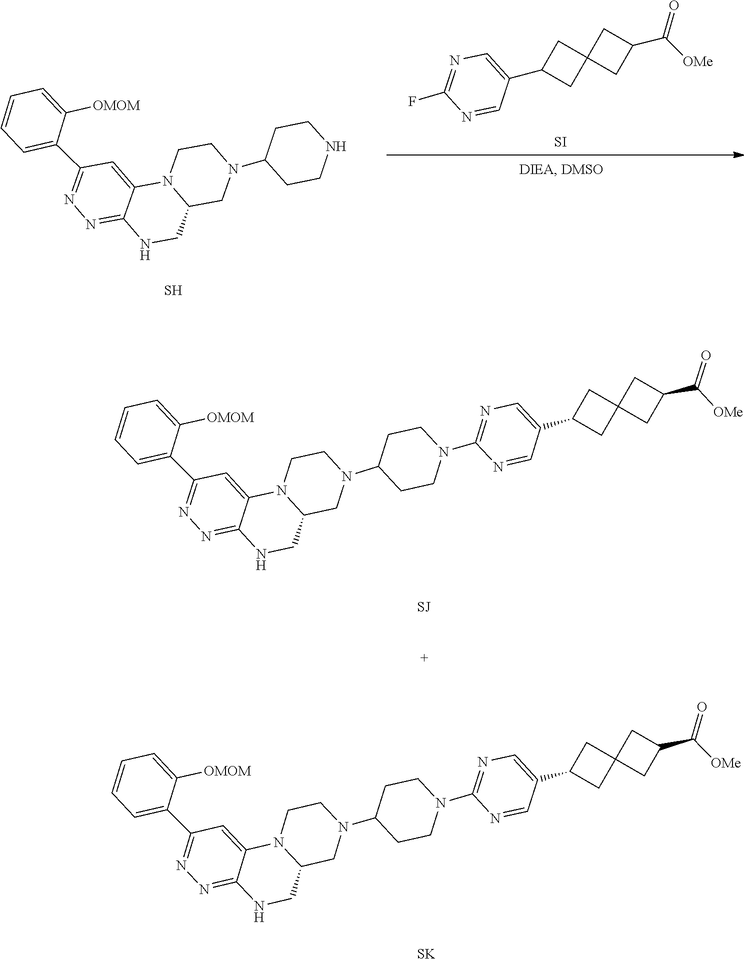

To a solution of (10S)-4-[2-(methoxymethoxy)phenyl]-12-(4-piperidyl)-1,5,6,8,12-pentazatricyclo[8.4.0.02,7]tetradeca-2,4,6-triene (600 mg, 1 mmol, Intermediate SH) and methyl 6-(2-fluoropyrimidin-5-yl)spiro[3.3]heptane-2-carboxylate (598 mg, 2.39 mmol, Intermediate SI) in DMSO (13 mL) was added DIEA (926 mg, 7.16 mmol, 1.25 mL). The mixture was then stirred at 60° C. for 12 hrs. On completion, the reaction mixture was filtered and concentrated under reduced pressure to give a residue. The crude product was purified by reversed-phase HPLC(0.1% FA condition) to give desired compound (600 mg) as a yellow solid. The compound was further separated by SFC (column: DAICEL CHIRALCEL OD(250 mm*30 mm,10 um);mobile phase: [ACN/MeOH(0.1% NH3H2O)];B %: 35%-35%, A10; 500 min) to give (S)-methyl 6-(2-(4-(2-(2-(methoxymethoxy)phenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylate (150 mg, peak 1) as a yellow solid (LC-MS (ESI⁺) m/z641.6 (M+H)⁺) and (S)-methyl 6-(2-(4-(2-(2-(methoxymethoxy)phenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylate (150 mg, peak 2) as a yellow solid (LC-MS (ESI⁺) m/z641.4 (M+H)⁺).

### (S)-6-(2-(4-(2-(2-hydroxyphenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylic acid (Intermediate SL)

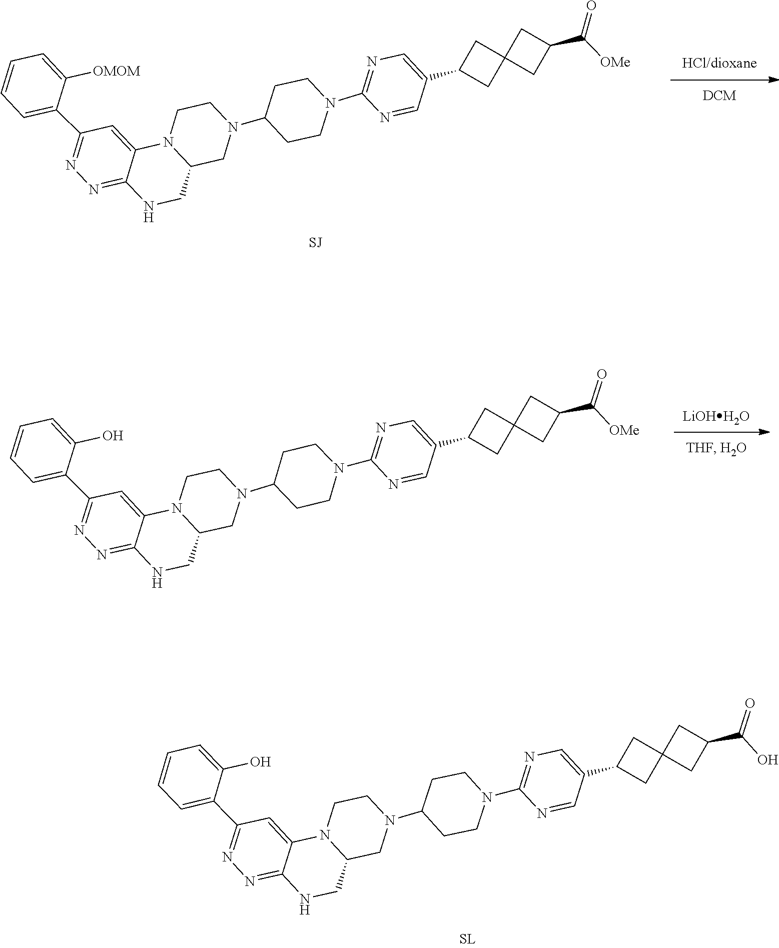

Step 1-(S)-methyl 6-(2-(4-(2-(2-hydroxyphenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylate. To a solution of methyl 6-[2-[4-[(1OS)-4-[2-(methoxymethoxy)phenyl]-1,5,6,8,12-pentazatricyclo[8.4.0.02,7]tetradeca-2,4,6-trien-12-yl]-1-piperidyl]pyrimidin-5-yl]spiro[3.3]heptane-2-carboxylate (150 mg, 234 umol, Intermediate SJ) in DCM (1.5 mL) was added HCl/dioxane (4 M, 0.7 mL). The mixture was then stirred at 25° C. for 2 hrs. On completion, the reaction mixture was concentrated under reduced pressure to give the title compound (170 mg, HCl) as a yellow solid. LC-MS (ESI⁺) m/z 597.5 (M+H)⁺.

Step 2-(S)-6-(2-(4-(2-(2-hydroxyphenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylic acid. To a solution of methyl 6-[2-[4-[(10S)-4-(2-hydroxyphenyl)-1,5,6,8,12-pentazatricyclo[8.4.0.02,7]tetradeca-2,4,6-trien-12-yl]-1-piperidyl]pyrimidin-5-yl]spiro[3.3]heptane-2-carboxylate (160 mg, 215 umol, HCl) in THF (0.5 mL) and H₂O (0.5 mL) was added LiOH·H₂O (90.4 mg, 2.15 mmol). The mixture was then stirred at 25° C. for 4 hrs. On completion, the reaction mixture was filtered and concentrated under reduced pressure to give a residue. The crude product was purified by reversed-phase HPLC 0.1% HCl condition) to give title compound (30 mg, 23% yield, HCl) as a white solid. LC-MS (ESI⁺) m/z 583.4 (M+H)⁺.

### (S)-6-(2-(4-(2-(2-hydroxyphenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylic acid (Intermediate SM)

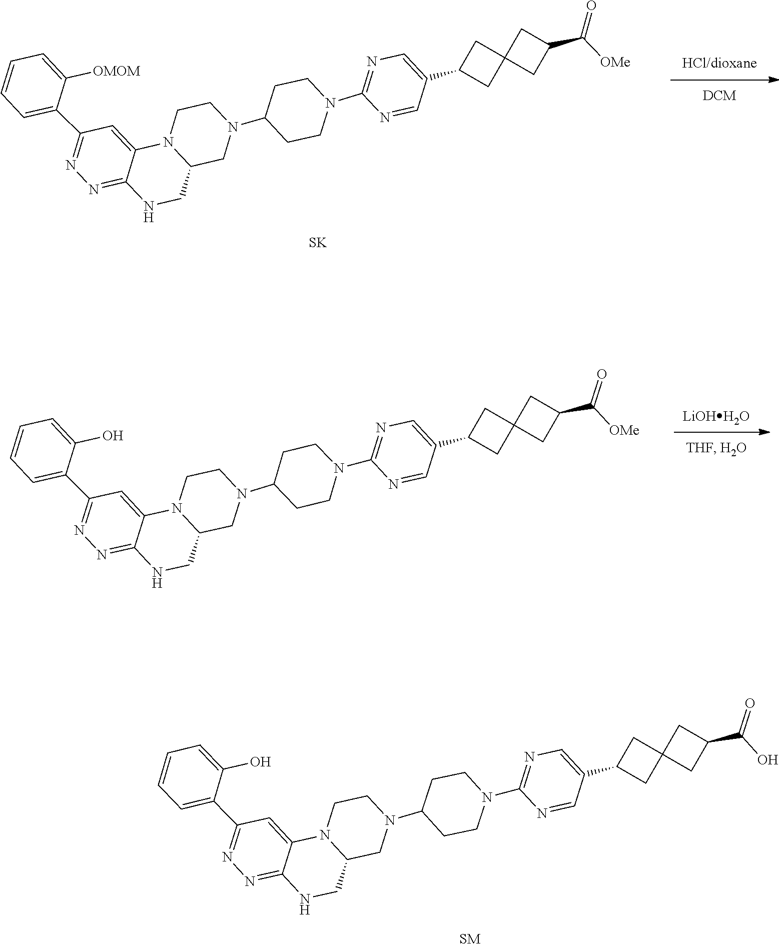

Step 1-(S)-methyl 6-(2-(4-(2-(2-hydroxyphenyl)-6a,7,9,10-tetrahydro-5H-pyrazino[1′,2′: 4,5]pyrazino[2,3-c]pyridazin-8(6H)-yl)piperidin-1-yl)pyrimidin-5-yl)spiro[3.3]heptane-2-carboxylate. To a solution of methyl 6-[2-[4-[(10S)-4-[2-(methoxymethoxy)phenyl]-1,5,6,8,12-pentazatricyclo[8.4.0.0^{2,7}]tetradeca-2,4,6-trien-12-yl]-1-piperidyl]pyrimidin-5-yl]spiro[3.3]heptane-2-carboxylate (150 mg, 234 umol, Intermediate SK) in DCM (1.5 mL) was added HCl/dioxane (4 M, 0.7 mL). The mixture was then stirred at 25° C. for 2 hrs. On completion, the reaction mixture was concentrated under reduced pressure to give the title compound (170 mg, HCl) as a yellow solid. LC-MS (ESI⁺) m/z 597.4 (M+H)⁺.

Step 2-(S)-6-(2-(4-(2-(2-hydroxyphenyl)-6a,7,9,10-tetrahydro-5H-pyrazino[1′,2′:4,5]pyrazino[2,3-c]pyridazin-8(6H)-yl)piperidin-1-yl)pyrimidin-5-yl)spiro[3.3]heptane-2-carboxylic acid. To a solution of methyl 6-[2-[4-[(10S)-4-(2-hydroxyphenyl)-1,5,6,8,12-pentazatricyclo[8.4.0.02,7]tetradeca-2,4,6-trien-12-yl]-1-piperidyl]pyrimidin-5-yl]spiro[3.3]heptane-2-carboxylate (160 mg, 220 umol, HCl) in THF (0.5 mL) and H₂O (0.5 mL) was added LiOH·H₂O (90.4 mg, 2.15 mmol). The mixture was then stirred at 25° C. for 4 hrs. On completion, the reaction mixture was filtered and concentrated under reduced pressure to give a residue. The crude product was purified by reversed-phase HPLC 0.1% HCl condition) to give title compound (100 mg, 63% yield, HCl) as a white solid. LC-MS (ESI⁺) m/z 583.3 (M+H)⁺.

### N—((R)-2-((tert-butyldimethylsilyl)oxy)-1-(5-ethynylpyridin-2-yl)ethyl)-2-methylpropane-2-sulfinamide (Intermediate SN) & N—((S)-2-((tert-butyldimethylsilyl)oxy)-1-(5-ethynylpyridin-2-yl)ethyl)-2-methylpropane-2-sulfinamide (Intermediate SO)

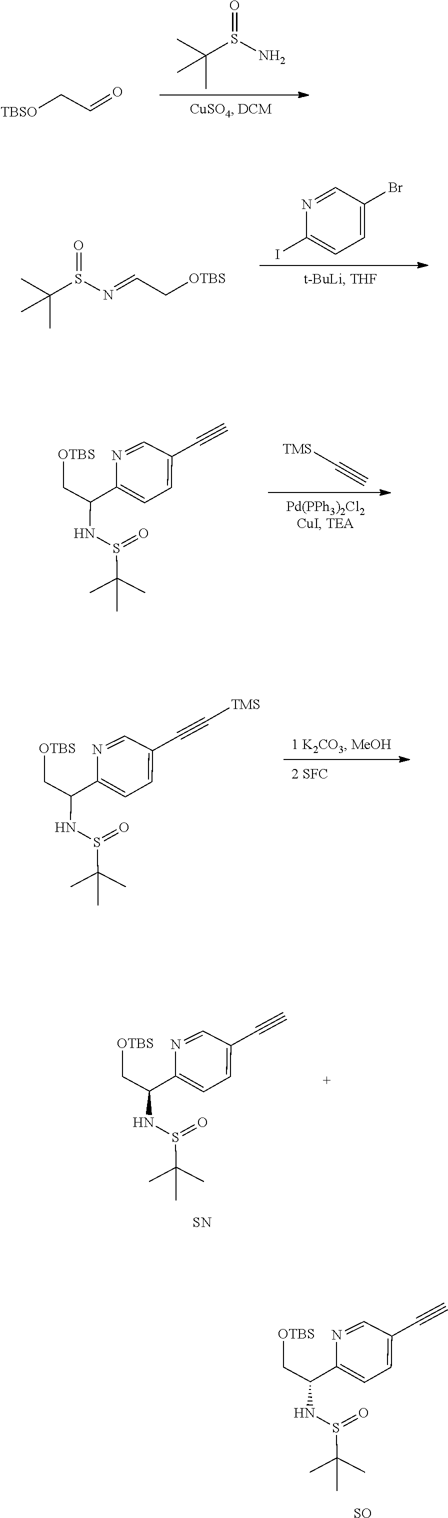

Step 1-(E)-N-(2-((tert-butyldimethylsilyl)oxy)ethylidene)-2-methylpropane-2-sulfinamide. To a solution of 2-((tert-butyldimethylsilyl)oxy)acetaldehyde (10 g, 57.4 mmol, CAS #102191-92-4) in DCM (150 mL) was added 2-methylpropane-2-sulfinamide (7.65 g, 63 mmol, CAS #146374-27-8) and CuSO₄ (27.5 g, 172 mmol). The mixture was then stirred at 20° C. for 12 hrs. On completion, the reaction mixture was filtered and concentrated under reduced pressure to give a residue. The residue was purified by column chromatography (SiO₂, Petroleum ether/Ethyl acetate=10/1 to 5/1) to give the title compound (12 g, 68% yield) as a yellow oil. LC-MS (ESI⁺) m/z 278.6 (M+H)⁺.

Step 2-N-(1-(5-bromopyridin-2-yl)-2-((tert-butyldimethylsilyl)oxy)ethyl)-2-methylpropane-2-sulfinamide. To a solution of 5-bromo-2-iodo-pyridine (5.12 g, 18 mmol, CAS #223463-13-6) in THF (80 mL) was added n-BuLi (2.5 M, 7.93 mL) slowly at −78° C., then (E)-N-(2-((tert-butyldimethylsilyl)oxy)ethylidene)-2-methylpropane-2-sulfinamide (5 g, 18 mmol) was added. The mixture was then stirred at −78° C. for 2 hrs. On completion, the reaction mixture was quenched with NH4C₁ (100 mL) at 0° C., and then diluted with H₂O (50 mL) and extracted with EA (100 mL×2). The combined organic layers were washed with aqueous NaCl (100 mL×2), dried over Na₂SO₄, filtered and concentrated under reduced pressure to give a residue. The residue was purified by column chromatography (SiO₂, Petroleum ether/Ethyl acetate=10/1 to 3/1) to give the title compound (1.8 g, 21% yield) as a yellow solid. LC-MS (ESI⁺) m/z 435.1(M+H)⁺.

Step 3-N-(2-((tert-butyldimethylsilyl)oxy)-1-(5-((trimethylsilyl)ethynyl)pyridin-2-yl)ethyl)-2-methylpropane-2-sulfinamide. A mixture of N-(1-(5-bromopyridin-2-yl)-2-((tert-butyldimethylsilyl)oxy)ethyl)-2-methylpropane-2-sulfinamide (1 g, 2.30 mmol), ethynyl(trimethyl)silane (2.26 g, 23 mmol, CAS #1066-54-2), CuI (87.5 mg, 459 umol) and Pd(PPh₃)₂Cl₂ (161 mg, 229 umol) in TEA (20 mL) was degassed and purged with N₂ three times. Then the mixture was stirred at 80° C. for 3 hrs under N₂ atmosphere. On completion, the reaction mixture was filtered and concentrated under reduced pressure to give a residue. The residue was purified by column chromatography (SiO₂, Petroleum ether/Ethyl acetate=10/1 to 5/1) to give the title compound (800 mg, 63% yield) as a yellow solid. LC-MS (ESI⁺) m/z 453.3(M+H)⁺.

Step 4-N—((R)-2-((tert-butyldimethylsilyl)oxy)-1-(5-ethynylpyridin-2-yl)ethyl)-2-methylpropane-2-sulfinamide & N—((S)-2-((tert-butyldimethylsilyl)oxy)-1-(5-ethynylpyridin-2-yl)ethyl)-2-methylpropane-2-sulfinamide. To a solution of N-(2-((tert-butyldimethylsilyl)oxy)-1-(5-((trimethylsilyl)ethynyl)pyridin-2-yl)ethyl)-2-methylpropane-2-sulfinamide (800 mg, 2 mmol) in MeOH (12 mL) was added K₂CO₃ (269 mg, 1.94 mmol). The mixture was then stirred at 20° C. for 2 hrs. On completion, the reaction mixture was concentrated under reduced pressure to remove solvent. The residue was diluted with H₂O (50 mL) and extracted with EA (50 mL×2). The combined organic layers were dried over Na₂SO₄, filtered and concentrated under reduced pressure to give a residue. The residue was triturated with EA at 20° C. for 10 min, then filtered to give an off-white solid. The solid separated by SFC (column: DAICEL CHIRALPAK IC(250 mm*30 mm, 10 um);mobile phase: [0.1% NH₃H₂O ETOH]; B %: 20%-20%, A2.4; 24 min) to give N—((R)-2-((tert-butyldimethylsilyl)oxy)-1-(5-ethynylpyridin-2-yl)ethyl)-2-methylpropane-2-sulfinamide (80 mg) as a white solid (¹H NMR (400 MHz, CHLOROFORM-d) δ=8.51 (d, J=2.0 Hz, 1H), 7.59-7.51 (m, 1H), 7.39 (d, J=8.0 Hz, 1H), 4.53-4.43 (m, 1H), 4.20 (d, J=2.4 Hz, 1H), 3.84-3.71 (m, 1H), 3.66-3.55 (m, 1H), 3.09 (s, 1H), 1.16 (s, 9H), 0.82 (s, 9H), −0.01 (d, J=11.2 Hz, 6H)) and N—((S)-2-((tert-butyldimethylsilyl)oxy)-1-(5-ethynylpyridin-2-yl)ethyl)-2-methylpropane-2-sulfinamide (80 mg) as an off-white solid (¹H NMR (400 MHz, CHLOROFORM-d) 6=8.51 (d, J=2.0 Hz, 1H), 7.62-7.51 (m, 1H), 7.39 (d, J=8.0 Hz, 1H), 4.54-4.45 (m, 1H), 4.20 (d, J=2.4 Hz, 1H), 3.81-3.72 (m, 1H), 3.64-3.55 (m, 1H), 3.10 (s, 1H), 1.16 (s, 9H), 0.82 (s, 9H), −0.01 (d, J=11.2 Hz, 6H). LC-MS (ESI⁺) m/z 381.3(M+H)⁺ for both isomers. Absolute configuration was assigned arbitrarily.

### (R)-2-amino-2-(5-ethynylpyridin-2-yl)ethanol (Intermediate SP)

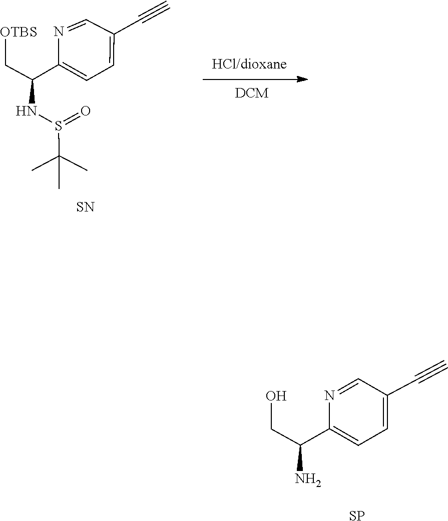

To a solution of N—((R)-2-((tert-butyldimethylsilyl)oxy)-1-(5-ethynylpyridin-2-yl)ethyl)-2-methylpropane-2-sulfinamide (30 mg, 78.8 umol, Intermediate SN) in DCM (1 mL) was added HCl/dioxane (4 M, 98.5 uL). The mixture was then stirred at 20° C. for 10 min. On completion, the reaction mixture was filtered and concentrated under reduced pressure to give the title compound (15 mg, HCl salt) as a white solid. LC-MS (ESI⁺) m/z 281.3(M+H)⁺.

### (S)-2-amino-2-(5-ethynylpyridin-2-yl)ethanol (Intermediate SQ)

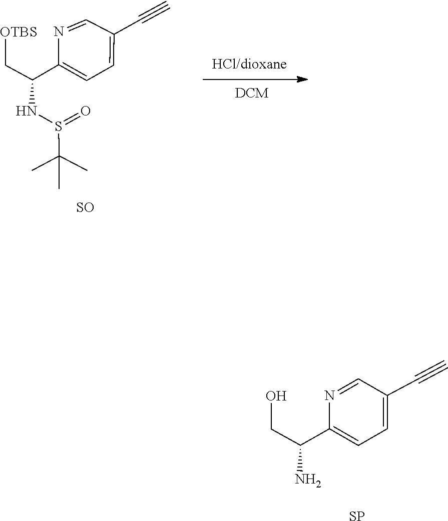

To a solution of N—((S)-2-((tert-butyldimethylsilyl)oxy)-1-(5-ethynylpyridin-2-yl)ethyl)-2-methylpropane-2-sulfinamide (30 mg, 78.8 umol, Intermediate SO) in DCM (1 mL) was added HCl/dioxane (4 M, 98.5 uL). The mixture was stirred at 20° C. for 10 min. On completion, the reaction mixture was filtered and concentrated under reduced pressure to give the title compound (15 mg, HCl salt) as a white solid. LC-MS (ESI⁺) m/z 281.3(M+H)⁺.

### (2S,4R)—N-(2-chloro-4-ethynylbenzyl)-4-hydroxypyrrolidine-2-carboxamide (Intermediate SR)

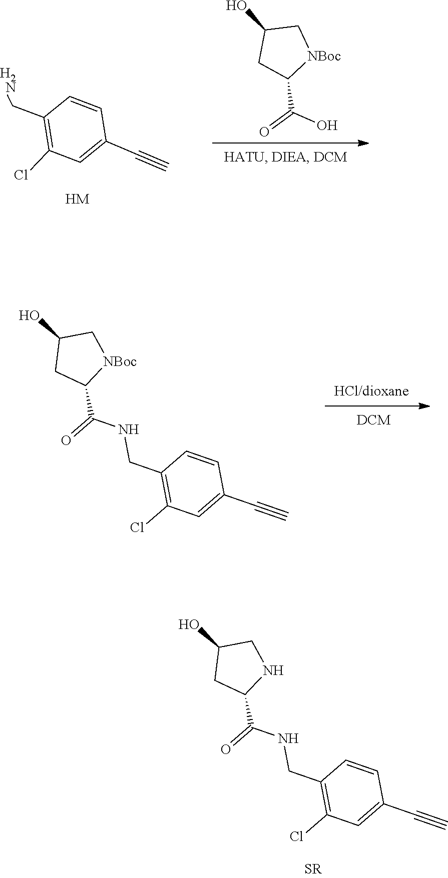

Step 1-(2S,4R)-tert-butyl 2-((2-chloro-4-ethynylbenzyl)carbamoyl)-4-hydroxypyrrolidine-1-carboxylate. To a solution of (2S,4R)-1-(tert-butoxycarbonyl)-4-hydroxypyrrolidine-2-carboxylic acid (2.85 g, 12.3 mmol, CAS #13726-69-7) in DCM (40 mL) was added EDCI (2.95 g, 15.4 mmol), HOAt (2.10 g, 15.4 mmol, 2.15 mL), DIEA (5.31 g, 41.0 mmol, 7.15 mL) and (2-chloro-4-ethynylphenyl)methanamine (1.7 g, 10.3 mmol, Intermediate HM) at 25° C. Then the mixture was stirred at 25° C. for 2 hrs. On completion, the reaction mixture was filtered and concentrated in vacuo to give the crude residue. The residue was purified by column chromatography (SiO₂, Petroleum ether/Ethyl acetate=1/1 to 0/1) to give the title compound (3.7 g, 68% yield) as a yellow solid. LC-MS (ESI⁺) m/z 279.2. (M−99)⁺.

Step 2-(2S,4R)—N-(2-chloro-4-ethynylbenzyl)-4-hydroxypyrrolidine-2-carboxamide. To a solution of (2S,4R)-tert-butyl 2-((2-chloro-4-ethynylbenzyl)carbamoyl)-4-hydroxypyrrolidine-1-carboxylate (3.6 g, 9.50 mmol) in DCM (30 mL) was added HCl/dioxane (4 M, 2.38 mL) at 25° C., then the mixture was stirred at 25° C. for 1 hr. On completion, the reaction mixture was filtered and concentrated in vacuo to give the title compound (2.8 g, HCl) as a yellow solid. LC-MS (ESI⁺) m/z 278.9 (M+H)⁺.

### (2S,4R)-1-((R)-2-amino-5-hydroxy-3,3-dimethylpentanoyl)-N-(2-chloro-4-ethynylbenzyl)-4-hydroxypyrrolidine-2-carboxamide (Intermediate SS) and (2S,4R)-1-((S)-2-amino-5-hydroxy-3,3-dimethylpentanoyl)-N-(2-chloro-4-ethynylbenzyl)-4-hydroxypyrrolidine-2-carboxamide (Intermediate ST)

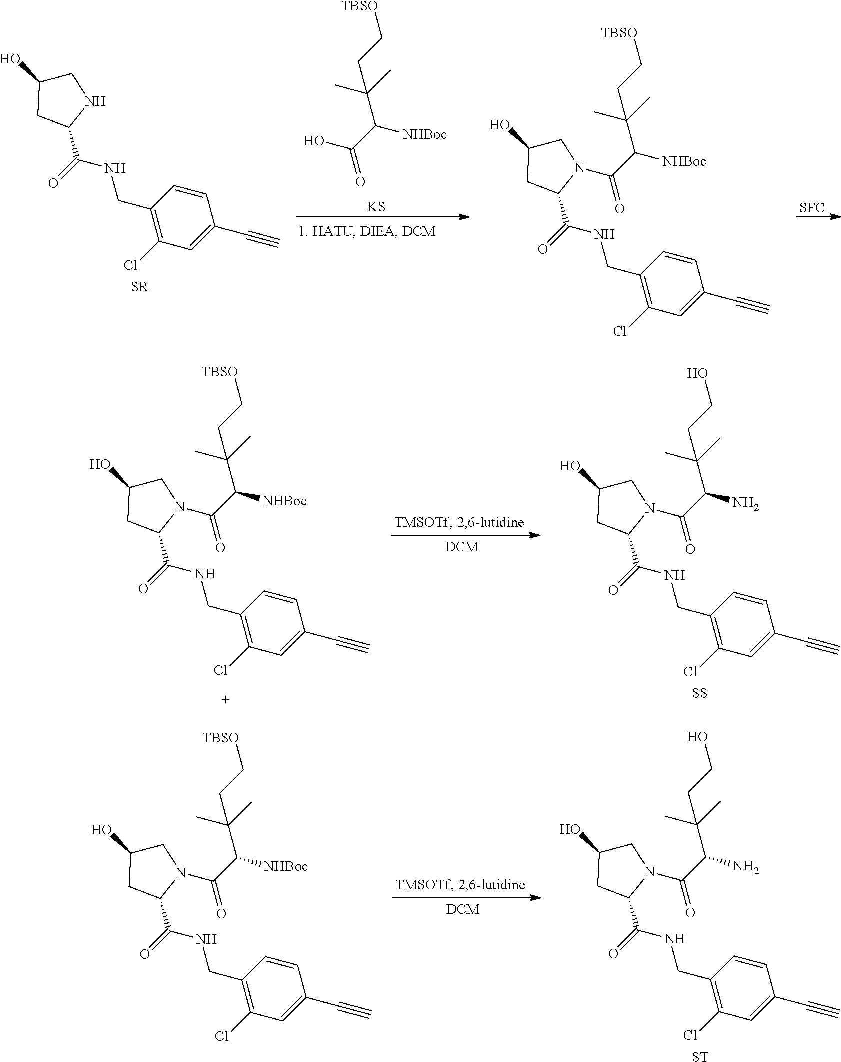

Step 1-Tert-butyl (5-((tert-butyldimethylsilyl)oxy)-1-((2S,4R)-2-((2-chloro-4-ethynylbenzyl)carbamoyl)-4-hydroxypyrrolidin-1-yl)-3,3-dimethyl-1-oxopentan-2-yl)carbamate. To a solution of 2-((tert-butoxycarbonyl)amino)-5-((tert-butyldimethylsilyl)oxy)-3,3-dimethylpentanoic acid (1.2 g, 3.20 mmol, Intermediate KS) in DCM (1 mL) was added HATU (1.82 g, 4.79 mmol), DIEA (1.24 g, 9.59 mmol, 1.67 mL) and (2S,4R)—N-(2-chloro-4-ethynylbenzyl)-4-hydroxypyrrolidine-2-carboxamide (980 mg, 3.11 mmol, HCl, Intermediate SR) at 25° C., then the mixture was stirred at 25° C. for 0.5 hr. On completion, the reaction mixture was filtered and concentrated in vacuo to give the crude residue. The crude product was purified by reversed-phase HPLC (0.1% NH₃•H₂O) to give the coupled product (700 mg, 33% yield) as a yellow solid. LC-MS (ESI⁺) m/z 636.9. (M+H)⁺.

Step 2-Tert-butyl N-[(1R)-4-[tert-butyl(dimethyl)silyl]oxy-1-[(2S,4R)-2-[(2-chloro-4-ethynyl-phenyl)methylcarbamoyl]-4-hydroxy-pyrrolidine-1-carbonyl]-2,2-dimethyl-butyl]carbamate and tert-butyl N-[(1S)-4-[tert-butyl(dimethyl)silyl]oxy-1-[(2S,4R)-2-[(2-chloro-4-ethynyl-phenyl)methylcarbamoyl]-4-hydroxy-pyrrolidine-1-carbonyl]-2,2-dimethyl-butyl]carbamate. Tert-butyl (5-((tert-butyldimethylsilyl)oxy)-1-((2S,4R)-2-((2-chloro-4-ethynylbenzyl)carbamoyl)-4-hydroxypyrrolidin-1-yl)-3,3-dimethyl-1-oxopentan-2-yl)carbamate (700 mg) was separated by SFC (column: DAICEL CHIRALPAK AD (250 mm*30 mm, 10 um); mobile phase: [0.1% NH₃H₂O IPA]; B %: 25%-25%, C6.0; 80 min) to give tert-butyl N-[(1R)-4-[tert-butyl(dimethyl)silyl]oxy-1-[(2S,4R)-2-[(2-chloro-4-ethynyl-phenyl)methylcarbamoyl]-4-hydroxy-pyrrolidine-1-carbonyl]-2,2-dimethyl-butyl]carbamate (350 mg, 50% yield, peak 1) an off white oil and tert-butyl N-[(1S)-4-[tert-butyl(dimethyl)silyl]oxy-1-[(2S,4R)-2-[(2-chloro-4-ethynyl-phenyl)methylcarbamoyl]-4-hydroxy-pyrrolidine-1-carbonyl]-2,2-dimethyl-butyl]carbamate (340 mg, 48% yield, peak 2) as an off white oil. Absolute stereochemistry was assigned arbitrarily.

Step 3-(2S,4R)-1-((R)-2-amino-5-hydroxy-3,3-dimethylpentanoyl)-N-(2-chloro-4-ethynylbenzyl)-4-hydroxypyrrolidine-2-carboxamide. To a solution of tert-butyl ((R)-5-((tert-butyldimethylsilyl)oxy)-1-((2S,4R)-2-((2-chloro-4-ethynylbenzyl)carbamoyl)-4-hydroxypyrrolidin-1-yl)-3,3-dimethyl-1-oxopentan-2-yl)carbamate (350 mg, 550 umol) in DCM (5 mL) was added TMSOTf (1.10 g, 4.95 mmol, 895 uL) and 2,6-dimethylpyridine (892 mg, 8.25 mmol, 970 uL) at 0° C., then the mixture was stirred at 25° C. for 1 hr. On completion, the reaction mixture was filtered and concentrated in vacuo to give the crude residue. The crude product was purified by reversed-phase HPLC (0.1% FA condition) to give the title compound (170 mg, 66% yield, FA) as a yellow solid. LC-MS (ESI⁺) m/z 536.4 (M+H)⁺.

Step 4-(2S,4R)-1-((S)-2-amino-5-hydroxy-3,3-dimethylpentanoyl)-N-(2-chloro-4-ethynylbenzyl)-4-hydroxypyrrolidine-2-carboxamide. To a mixture of tert-butyl ((S)-5-((tert-butyldimethylsilyl)oxy)-1-((2S,4R)-2-((2-chloro-4-ethynylbenzyl)carbamoyl)-4-hydroxypyrrolidin-1-yl)-3,3-dimethyl-1-oxopentan-2-yl)carbamate (350 mg, 550 umol) in DCM (75 mL) was added 2,6-dimethylpyridine (884 mg, 8.25 mmol) and TMSOTf (1.10 g, 4.95 mmol) at 0° C. The mixture was stirred at 25° C. for 1 hr. On completion, the reaction mixture was filtered and concentrated under reduced pressure to give a residue. The residue was purified by prep-HPLC (FA condition) to give the title compound (222 mg, 85% yield, FA) as a white solid. LC-MS (ESI+) m/z 422.3 (M+H)⁺.

### (S)-4-(2-(2-(2-(methoxymethoxy)phenyl)-6a,7,9,10-tetrahydro-5H-pyrazino[1′,2′:4,5]pyrazino[2,3-c]pyridazin-8(6H)-yl)pyrimidin-5-yl)benzaldehyde (Intermediate SU)

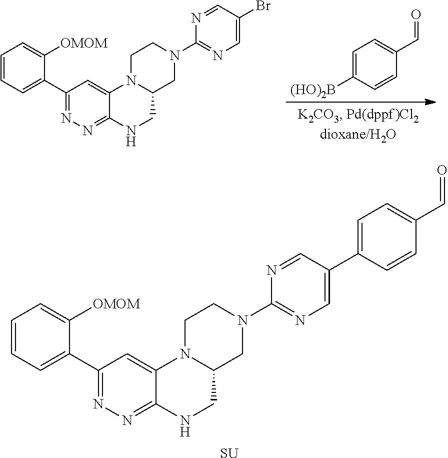

To A mixture of (1OS)-12-(5-bromopyrimidin-2-yl)-4-[2-(methoxymethoxy)phenyl]-1,5,6,8,12-pentazatricyclo[8.4.0.02,7]tetradeca-2,4,6-triene (500 mg, 1.03 mmol, synthesized via Step 1 of Intermediate EA), (4-formylphenyl)boronic acid (186 mg, 1.24 mmol, CAS #87199-17-5), K₂CO₃ (428 mg, 3.10 mmol) and Pd(dppf)C₁₂ (75.5 mg, 103 umol) in dioxane (8 mL) and H₂O (2 mL) was degassed and purged with N₂ three times. Then the mixture was stirred at 90° C. for 4 hrs under N₂ atmosphere. On completion, the reaction mixture was quenched with water (20 mL) and extracted with DCM (3×20 mL). The combined organic layer was washed with brine (20 mL) and dried over anhydrous sodium sulfate, filtered and concentrated in vacuo to give the crude residue. The residue was purified by silica gel chromatography (DCM:MeOH=1:0 to 10:1) to give the title compound (400 mg, 73% yield) as a yellow solid. LC-MS (ESI⁺) m/z 510.2 (M+H)⁺.

### (5-Ethynyl-3-methoxypyridin-2-yl)methanamine (Intermediate SV)

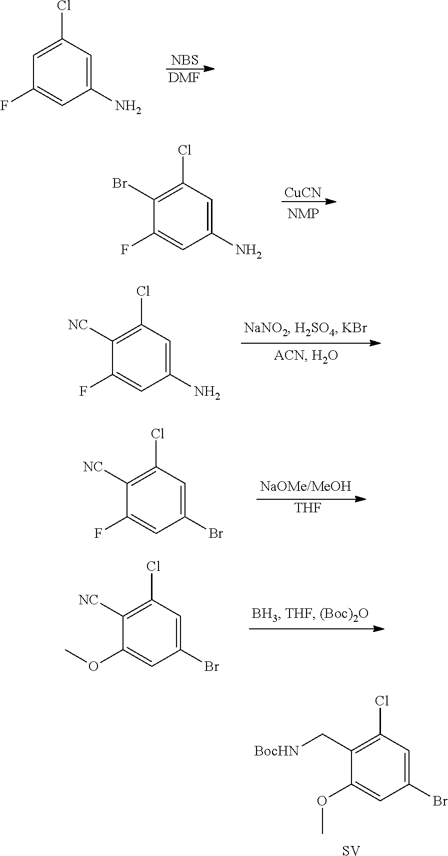

Step 1-4-bromo-3-chloro-5-fluoroaniline. To a solution of 3-chloro-5-fluoro-aniline (15 g, 103 mmol, CAS #4863-91-6) in ACN (450 mL) was added NBS (20.2 g, 113.35 mmol) at 0° C. The mixture was stirred at 20° C. for 3 hrs. On completion, the reaction was diluted with water (500 mL), concentrated in vacuo, then extracted with EtOAc (150 mL×3). The combined organic layer was washed with brine (200 mL), dried over Na₂SO₄, filtered and concentrated in vacuo. Then the crude was purified by silica gel column chromatography (Petroleum ether: EtOAc from 50:1 to 10:1) to afford the title compound (23 g, 89% yield) as a yellow solid. LC-MS (ESI⁺) m/z 225.9 (M+H)⁺.

Step 2-4-amino-2-chloro-6-fluorobenzonitrile. To a solution of 4-bromo-3-chloro-5-fluoro-aniline (23 g, 102.5 mmol) in NMP (200 mL) was added CuCN (9.2 g, 103 mmol). The mixture was stirred at 140° C. for 16 hrs. On completion, the reaction was poured into NH₃.H₂O (12%, 800 mL) and stirred for 4 h. Then the mixture was then filtered to afford a brown solid as the title compound (12.5 g, 61% yield). LC-MS (ESI⁺) m/z 171.1 (M+H)⁺.

Step 3-4-bromo-2-chloro-6-fluorobenzonitrile. To a solution of 4-amino-2-chloro-6-fluoro-benzonitrile (12.5 g, 73.3 mmol) and H₂SO₄ (21.6 g, 219.9 mmol) in H₂O (40 mL) and ACN (120 mL) was added a solution of NaNO₂ (5.56 g, 80.61 mmol) in H₂O (40 mL) and a solution of KBr (17.4 g, 146.6 mmol) in H₂O (40 mL) at 0° C. The mixture was stirred at 20° C. for 2 hrs. On completion, the reaction was diluted with water (500 mL), and extracted with EtOAc (150 mL×3). The combined organic layer was washed with brine (200 mL), dried over Na₂SO₄, filtered and concentrated in vacuo. Then the crude was purified by silica gel column chromatography (Petroleum ether: EtOAc from 100:1 to 5:1) to afford the title compound (8 g, 42% yield) as a yellow solid. LC-MS (ESI⁺) m/z 235.9 (M+H)⁺.

Step 4-4-bromo-2-chloro-6-methoxybenzonitrile. To a solution of 4-bromo-2-chloro-6-fluoro-benzonitrile (6.8 g, 29 mmol) in THF (70 mL) was added sodium methanolate (5.4 M, 5.91 mL, 30% solution) at 0° C. The mixture was then stirred at 20° C. for 16 hrs. On completion, the reaction was concentrated in vacuo. Then the crude was purified by silica gel column chromatography (Petroleum ether: EtOAc from 100:1 to 10:1) to afford the title compound (6.1 g, 77% yield) as a yellow oil. LC-MS (ESI⁺) m/z 248.0 (M+H)⁺. ¹H NMR (400 MHz, DMSO-d₆) δ=7.62 (d, J=1.6 Hz, 1H), 7.54 (d, J=1.6 Hz, 1H), 3.97 (s, 3H).

Step 5-(5-ethynyl-3-methoxypyridin-2-yl)methanamine. BH₃.THF (1 M, 115.6 mL) was cooled to 5° C., then 4-bromo-2-chloro-6-methoxy-benzonitrile (5.7 g, 23.1 mmol) in THF (100 mL) was added. The mixture was then stirred at 60° C. for 16 hrs. Next, the reaction was quenched with H₂O (2 mL), then (Boc₂)O (7.6 g, 34.7 mmol) was added and the mixture was stirred at 20° C. for 2 hours. On completion, the reaction was quenched with water (150 mL), and extracted with EtOAc (60 mL×3). The combined organic layer was washed with brine (100 mL), dried over Na₂SO₄, filtered and concentrated in vacuo. Then the crude was purified by reversed phase HPLC (0.1% FA condition) to afford the title compound (3.9 g, 38% yield,) as a yellow solid. LC-MS (ESI⁺) m/z 295.9 (M-56)⁺.

### (S)-2-(8-(piperidin-4-ylmethyl)-6,6a,7,8,9,10-hexahydro-5H-pyrazino[1′,2′:4,5]pyrazino[2,3-c]pyridazin-2-yl)phenol (Intermediate SW)

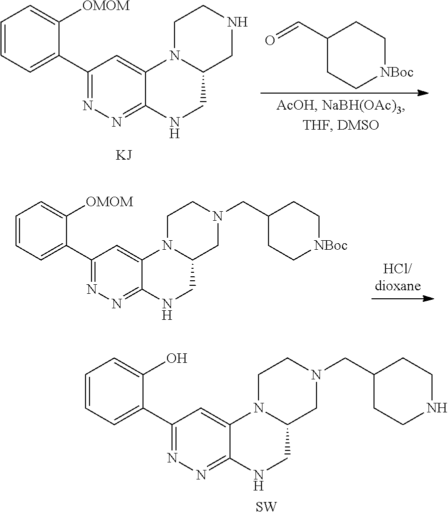

Step 1-(S)-tert-butyl 4-((2-(2-(methoxymethoxy)phenyl)-6a,7,9,10-tetrahydro-5H-pyrazino[1′,2′:4,5]pyrazino[2,3-c]pyridazin-8(6H)-yl)methyl)piperidine-1-carboxylate. To a solution of (10R)-4-[2-(methoxymethoxy)phenyl]-1,5,6,8,12-pentazatricyclo[8.4.0.02,7]tetradeca-2,4,6-triene (1.5 g, 4.58 mmol, Intermediate KJ) in THF (20 mL) and DMSO (4 mL) was added AcOH (825 mg, 13.8 mmol, 786 uL) and tert-butyl 4-formylpiperidine-1-carboxylate (1.17 g, 5.50 mmol, CAS #137076-22-3) at 25° C. and the mixture was stirred for 1 hour. Then NaBH(OAc)₃ (2.91 g, 13.8 mmol) was added at 0° C. and the mixture was stirred at 25° C. for 3 hrs. On completion, the reaction mixture was concentrated under reduced pressure to give a residue. The crude product was purified by reversed-phase HPLC (FA) to give the title compound (2 g, 49% yield) as a yellow solid. LC-MS (ESI⁺) m/z 525.2 (M+H)⁺.

Step 2-(S)-2-(8-(piperidin-4-ylmethyl)-6,6a,7,8,9,10-hexahydro-5H-pyrazino[1′,2′:4,5]pyrazino[2,3-c]pyridazin-2-yl)phenol. A solution of tert-butyl 4-[[(10S)-4-[2-(methoxymethoxy)phenyl]-1,5,6,8,12-pentazatricyclo[8.4.0.02,7]tetradeca-2,4,6-trien-12-yl]methyl]piperidine-1-carboxylate (1.9 g, 3.62 mmol) in HCl/dioxane (20 mL) was stirred at 25° C. for 2 hrs. On completion, the reaction was concentrated under reduced pressure to give the residue. The residue was purified by prep-HPLC (HCl) to give the title compound (1.5 g, 87% yield) as a yellow solid. LC-MS (ESI⁺) m/z 381.3 (M+H)⁺.

### (S)-2-(8-([1,4′:1′,4″-terpiperidin]-4-ylmethyl)-6,6a,7,8,9,10-hexahydro-5H-pyrazino[1′,2′:4,5]pyrazino[2,3-c]pyridazin-2-yl)phenol (Intermediate SX)

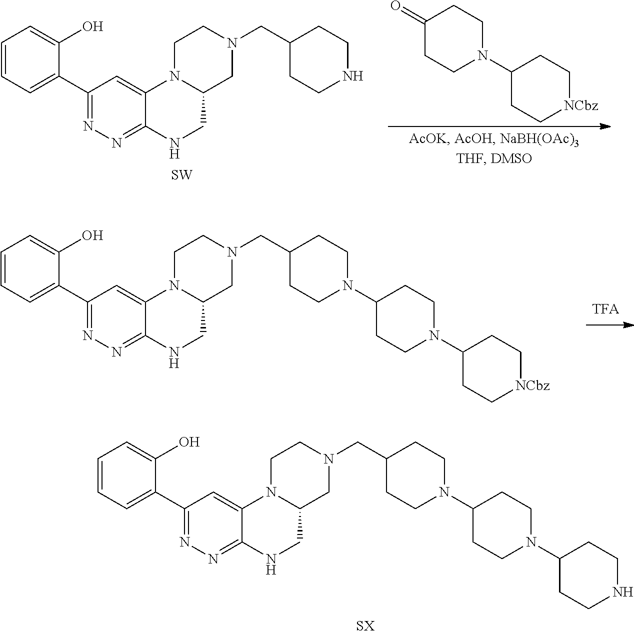

Step 1-(S)-benzyl 4-((2-(2-hydroxyphenyl)-6a,7,9,10-tetrahydro-5H-pyrazino[1′,2′:4,5]pyrazino[2,3-c]pyridazin-8(6H)-yl)methyl)-[1,4′:1′,4″-terpiperidine]-1″-carboxylate. To a solution of 2-[(1OS)-12-(4-piperidylmethyl)-1,5,6,8,12-pentazatricyclo[8.4.0.02,7]tetradeca-2,4,6-trien-4-yl]phenol (640 mg, 1.68 mmol, Intermediate SW) in THF (10 mL) and DMSO (5 mL) was added AcOK (825 mg, 8.41 mmol) at 25° C. for 1.5 hrs. Next, AcOH (303 mg, 5.05 mmol, 289 uL) and benzyl 4-(4-oxo-1-piperidyl) piperidine-1-carboxylate (1.33 g, 4.21 mmol, CAS #880462-12-4) was added at 25° C. and the mixture was stirred for 1.5 hours. At last, NaBH(OAc)₃ (1.07 g, 5.05 mmol) was added at 0° C., then the mixture was stirred at 25° C. for 10 hrs. On completion, the reaction mixture was quenched with H₂O (2 mL) and concentrated under reduced pressure to give a residue. The crude product was purified by reversed-phase HPLC (NH₃•H₂O) then purified further by reversed-phase HPLC (HCl) to give the title compound (200 mg, 99% purity, 16% yield) as a yellow solid. LC-MS (ESI⁺) m/z 681.5 (M+H)⁺.

Step 4-(S)-2-(8-([1,4′:1′,4″-terpiperidin]-4-ylmethyl)-6,6a,7,8,9,10-hexahydro-5H-pyrazino[1′,2′:4,5]pyrazino[2,3-c]pyridazin-2-yl)phenol. A solution of benzyl 4-[4-[4-[[(1OS)-4-(2-hydroxyphenyl)-1,5,6,8,12-pentazatricyclo[8.4.0.02,7]tetradeca-2,4,6-trien-12-yl]methyl]-1-piperidyl]-1-piperidyl]piperidine-1-carboxylate (100 mg, 147 umol) in TFA (3 mL) was stirred at 25° C. for 2 hrs. On completion, the reaction was concentrated under reduced pressure to give the residue. The residue was purified by prep-HPLC (FA) to give the title compound (85 mg, 97% yield) as a white solid. LC-MS (ESI⁺) m/z 547.3 (M+H)⁺.

### 3-methyl-2-(3-(2-oxoethoxy)isoxazol-5-yl)butanoic acid (Intermediate SY)

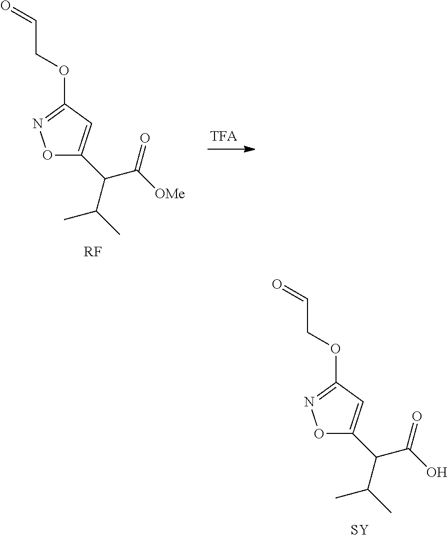

A solution of ethyl 2-[3-(2, 2-diethoxyethoxy) isoxazol-5-yl]-3-methyl-butanoate (300 mg, 911 umol, Intermediate RF) in TFA (6 mL) was stirred at 25° C. for 2 hrs. On completion, the reaction mixture was diluted with H₂O (1 mL) and extracted with EA 15 mL (5 mL×3). The combined organic layers were washed with aqueous NaCl (5 mL×3), dried over Na₂SO₄, filtered and concentrated under reduced pressure to give the title compound (300 mg) as a white oil. LC-MS (ESI⁺) m/z 228.0 (M+H)⁺.

### 2-(3-(2-(4-(((S)-2-(2-hydroxyphenyl)-6a,7,9,10-tetrahydro-5H-pyrazino[1′,2′:4,5]pyrazino[2,3-c]pyridazin-8(6H)-yl)methyl)-[1,4′:1′,4″-terpiperidin]-1″-yl)ethoxy)isoxazol-5-yl)-3-methylbutanoic acid (Intermediate SZ)

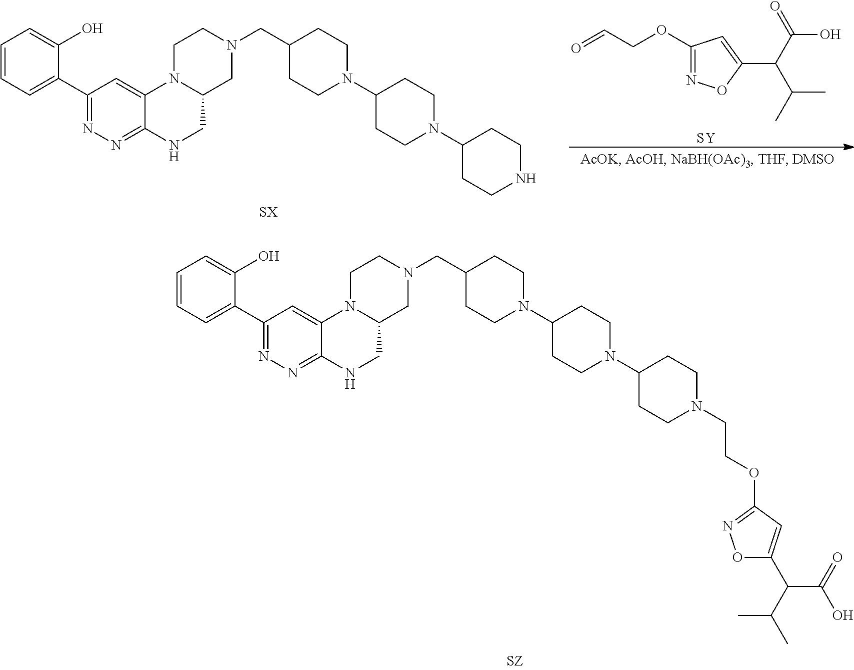 [SMILES: Oc1ccccc1-c1cc2c(nn1)NC[C@H]1CN(CC3CCN(C4CCN(C5CCNCC5)CC4)CC3)CCN21 ; CC(C)C(C(=O)O)c1cc(OCC=O)no1 ; "SY" ; CC(C)C(C(=O)O)c1cc(OCCN2CCC(N3CCC(N4CCC(CN5CCN6c7cc(-c8ccccc8O)nnc7NC[C@H]6C5)CC4)CC3)CC2)no1]

To a solution of 2-[(1OS)-12-[[1-[1-(4-piperidyl)-4-piperidyl]-4-piperidyl]methyl]-1,5,6,8,12-pentazatricyclo[8.4.0.02,7]tetradeca-2,4,6-trien-4-yl]phenol (65 mg, 110 umol, Intermediate SX, FA) in THF (4 mL), DMSO (2 mL) was added AcOK (53.8 mg, 548 umol) at 25° C. for 0.5 hours. Then AcOH (19.8 mg, 329 umol, 18.8 uL), and 3-methyl-2-[3-(2-oxoethoxy) isoxazol-5-yl]butanoic acid (32.4 mg, 143 umol, Intermediate SY) was added at 25° C. and the mixture was stirred for 1.5 hrs. At last, NaBH(OAc)₃ (69.7 mg, 329 umol) was added at 0° C. and the mixture was stirred at 25° C. for 12 hrs. On completion, the reaction was concentrated under reduced pressure to give the residue. The residue was purified by prep-HPLC (NH₃•H₂O) to give the title compound (75 mg, 89% yield) as a white solid. LC-MS (ESI⁺) m/z 758.5 (M+H)⁺.

### Tert-butyl (3-chloro-4-ethynylbenzyl)carbamate (Intermediate TB)

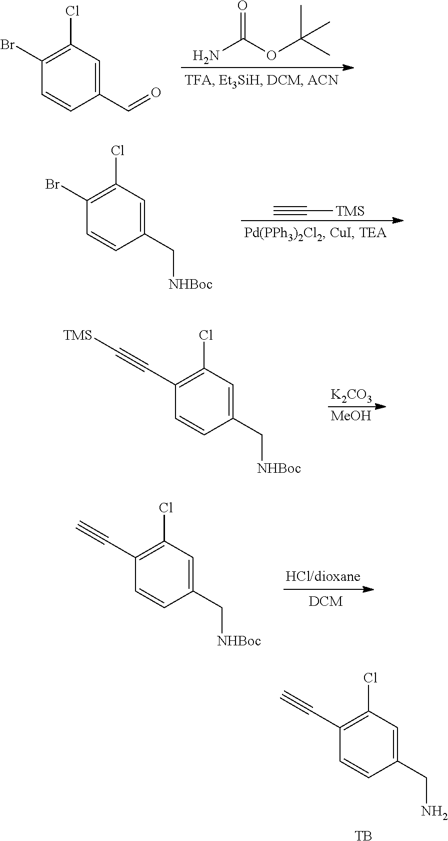

Step 1-Tert-butyl 4-bromo-3-chlorobenzylcarbamate. To a solution of 4-bromo-3-chloro-benzaldehyde (5 g, 22.8 mmol, CAS #120077-69-2) in DCM (30 mL) and ACN (90 mL) was added NH₂Boc (8.01 g, 68.4 mmol, CAS #4248-19-5), Et₃SiH (7.95 g, 68.4 mmol) and dropwise addition of TFA (5.20 g, 45.6 mmol, 3.37 mL). The mixture was then stirred at 25° C. for 12 hrs. On completion, the reaction mixture was quenched with aq. NaHCO₃ (30 mL) and extracted with DCM (50 mL×3). The combined organic layers were washed with brine (30 mL), dried over Na₂SO₄, filtered and concentrated under reduced pressure to give a residue. The residue was purified by column chromatography (SiO₂, Petroleum ether/Ethyl acetate=50/1 to 30/1) to give title compound (2 g, 24% yield) as a white solid. LC-MS (ESI⁺) m/z265.8 (M−55)⁺.

Step 2-Tert-butyl 3-chloro-4-((trimethylsilyl)ethynyl)benzylcarbamate. To a solution of tert-butyl N-[(4-bromo-3-chloro-phenyl)methyl]carbamate (0.7 g, 2.18 mmol) and ethynyl(trimethyl)silane (2.14 g, 21.8 mmol, CAS #1066-54-2) in TEA (14 mL) was added CuI (83.2 mg, 437 umol) and Pd(PPh₃)₂Cl₂ (153 mg, 218 umol). The mixture was stirred at 60° C. for 12 hrs. On completion, the reaction mixture was quenched with H₂O (2 mL) at and extracted with EA (20 mL×3). The combined organic layers were washed with brine (2 mL), dried over Na₂SO₄, filtered and concentrated under reduced pressure to give a residue. The residue was purified by column chromatography (SiO₂, Petroleum ether/Ethyl acetate=50/1 to 15/1) to give title compound (550 mg, 17% yield) as a yellow oil. LC-MS (ESI⁺) m/z282 (M−55)⁺.

Step 3-Tert-butyl 3-chloro-4-ethynylbenzylcarbamate. To a solution of tert-butyl N-[[3-chloro-4-(2-trimethylsilylethynyl)phenyl]methyl]carbamate (500 mg, 340 umol) in MeOH (5 mL) was added K₂CO₃ (102 mg, 739 umol). The mixture was stirred at 25° C. for 1 hr. On completion, the reaction mixture was filtered and concentrated under reduced pressure to give a residue. The crude product was purified by reversed-phase HPLC(0.1% FA condition) to give title compound (40 mg, 18% yield) as a yellow oil. LC-MS (ESI⁺) m/z 265.8 (M+H)⁺.

Step 4-Tert-butyl (3-chloro-4-ethynylbenzyl)carbamate. To a solution of tert-butyl N-[(3-chloro-4-ethynyl-phenyl)methyl]carbamate (40 mg, 151 umol) in DCM (1 mL) was added HCl/dioxane (4 M, 0.2 mL). The mixture was stirred at 25° C. for 3 hrs. On completion, the reaction mixture was concentrated under reduced pressure to give the title compound (30 mg) as a yellow solid. LC-MS (ESI⁺) m/z149.3 (M+H)⁺.

### (4-Ethynyl-3-fluorophenyl)methanamine (Intermediate TC)

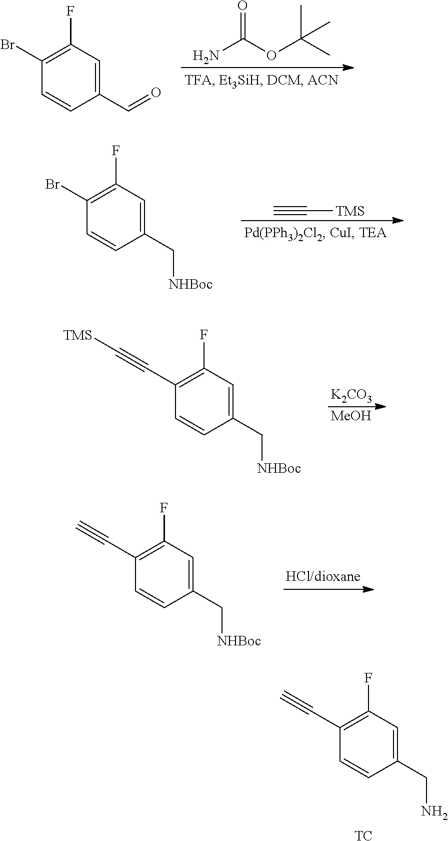

Step 1-Tert-butyl 4-bromo-3-fluorobenzylcarbamate. To a solution of 4-bromo-3-fluoro-benzaldehyde (10 g, 49.2 mmol, CAS #133059-43-5) in DCM (40 mL) and ACN (120 mL) was added Et₃SiH (17.18 g, 147.78 mmol) and tert-butyl carbamate (17.3 g, 147.8 mmol) and TFA (11.2 g, 98.5 mmol). The mixture was then stirred at 25° C. for 12 hrs. On completion, the reaction mixture was quenched with NaHCO₃ until the pH=7, and then diluted with EA (50 mL) and extracted with EA (150 mL×3). The combined organic layers were washed with NaCl (50 mL×3), dried over Na₂SO₄, filtered and concentrated under reduced pressure to give a residue. The residue was purified by column chromatography (SiO2, Petroleum ether/Ethyl acetate=100/1 to 20/1) to give the title compound (1.7 g, 11% yield) as a white solid. LC-MS (ESI⁺) m/z 247.8 (M−56)⁺.

Step 2-Tert-butyl 3-fluoro-4-((trimethylsilyl)ethynyl)benzylcarbamate. To a solution of tert-butyl N-[(4-bromo-3-fluoro-phenyl)methyl]carbamate (1.6 g, 5.26 mmol) in TEA (16 mL) was added CuI (200 mg, 1.05 mmol), Pd(PPh₃)₂Cl₂ (369 mg, 526 umol) and ethynyl(trimethyl)silane (7.75 g, 78.9 mmol, CAS #1066-54-2). The mixture was stirred at 80° C. for 12 hrs. On completion, H₂O (50 mL) was added and the mixture was filtered and extracted with ethyl acetate (50 mL×3). The combined organic layer was dried over anhydrous sodium sulfate, filtered and concentrated in vacuo to give the crude residue. The residue was purified by column chromatography (SiO₂, Petroleum ether/Ethyl acetate=100/1 to 20/1) to give the title compound (1.5 g, 13% yield) as a yellow oil. LC-MS (ESI⁺) m/z 266.2 (M−56+1)⁺.

Step 3-Tert-butyl 4-ethynyl-3-fluorobenzylcarbamate. To a solution of tert-butyl N-[[3-fluoro-4-(2-trimethylsilylethynyl)phenyl]methyl]carbamate (1.4 g, 4.36 mmol) in MeOH (15 mL) was added K₂CO₃ (601 mg, 4.36 mmol). The mixture was stirred at 25° C. for 0.5 hrs. On completion, the reaction mixture was quenched with NH4C₁ (30 mL) at 25° C., and then extracted with EA (30 mL×3). The combined organic layers were washed with NaCl (30 mL×3), dried over Na₂SO₄, filtered and concentrated under reduced pressure to give a residue. The residue was purified by reversed-phase HPLC(0.1% FA condition). After prep. HPLC purification, the eluent was concentrated to remove organic solvents. The residual aqueous solution was lyophilized to give the title compound (300 mg, 27% yield) as a white solid. LC-MS (ESI⁺) m/z 194.3 (M−56+1)⁺.

Step 4-(4-Ethynyl-3-fluorophenyl)methanamine. To a solution of tert-butyl N-[(4-ethynyl-3-fluoro-phenyl)methyl]carbamate (50 mg, 200 umol) in DCM (0.5 mL) was added HCl/dioxane (4 M, 0.1 mL). The mixture was stirred at 25° C. for 2 hrs. The reaction mixture was concentrated under reduced pressure to give the title compound (40 mg, HCl) as a white solid. LC-MS (ESI⁺) m/z 132.9 (M−14)⁺.

### Phenyl ((R)-1-((2S,4R)-2-((2-chloro-4-ethynylbenzyl)carbamoyl)-4-hydroxypyrrolidin-1-yl)-5-hydroxy-3,3-dimethyl-1-oxopentan-2-yl)carbamate (Intermediate TO)

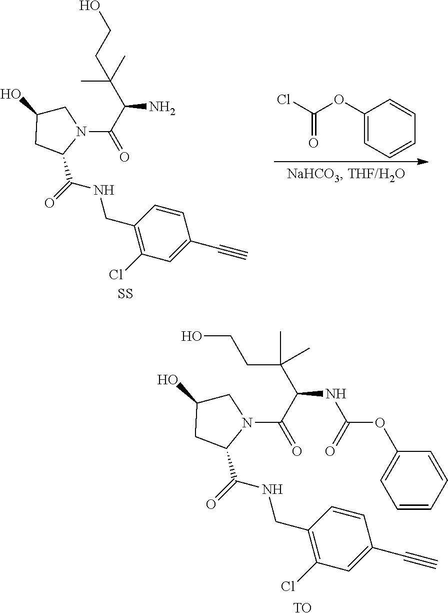

To a solution of (2S,4R)-1-((R)-2-amino-5-hydroxy-3,3-dimethylpentanoyl)-N-(2-chloro-4-ethynylbenzyl)-4-hydroxypyrrolidine-2-carboxamide (100 mg, 237 umol, Intermediate SS) in THF (2 mL) and H₂O (1 mL) was added NaHCO₃ (79.6 mg, 948 umol, 36.9 uL) and phenyl carbonochloridate (37.1 mg, 237 umol, 29.7 uL) at 0° C., then the mixture was stirred at 25° C. for 2 hrs. On completion, the reaction mixture was filtered and concentrated in vacuo to give the crude residue. The residue was purified by prep-TLC (Petroleum ether: Ethyl acetate=0:1) to give the title compound (65 mg, 48% yield) as a yellow solid. LC-MS (ESI⁺) m/z 542.3. (M+H)⁺.

### Phenyl ((S)-1-((2S,4R)-2-((2-chloro-4-ethynylbenzyl)carbamoyl)-4-hydroxypyrrolidin-1-yl)-5-hydroxy-3,3-dimethyl-1-oxopentan-2-yl)carbamate (Intermediate TP)

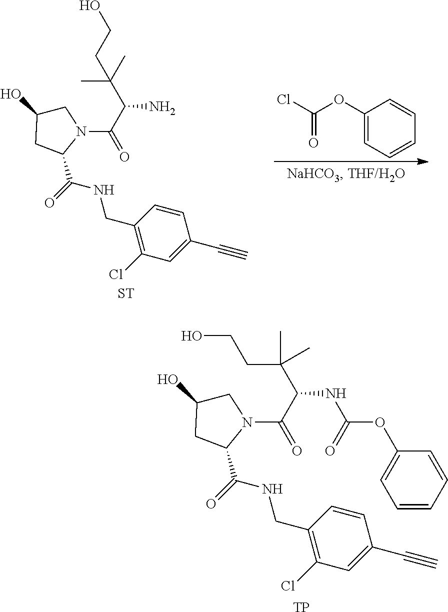

To a solution of (2S,4R)-1-((S)-2-amino-5-hydroxy-3,3-dimethylpentanoyl)-N-(2-chloro-4-ethynylbenzyl)-4-hydroxypyrrolidine-2-carboxamide (100 mg, 237 umol, Intermediate ST) in THF (2 mL) and H₂O (1 mL) was added NaHCO₃ (79.6 mg, 948 umol, 36.9 uL) and phenyl carbonochloridate (37.1 mg, 237 umol, 29.7 uL) at 0° C., then the mixture was stirred at 25° C. for 2 hrs. On completion, the reaction mixture was filtered and concentrated in vacuo to give the crude residue. The residue was purified by prep-TLC (Petroleum ether: Ethyl acetate=0:1) to give the title compound (70 mg, 54% yield) as a yellow solid. LC-MS (ESI⁺) m/z 542.3. (M+H)⁺.

### Phenyl ((S)-1-((2S,4R)-2-((2-chloro-4-ethynylbenzyl)carbamoyl)-4-hydroxypyrrolidin-1-yl)-3,3-dimethyl-1-oxobutan-2-yl)carbamate (Intermediate TQ)

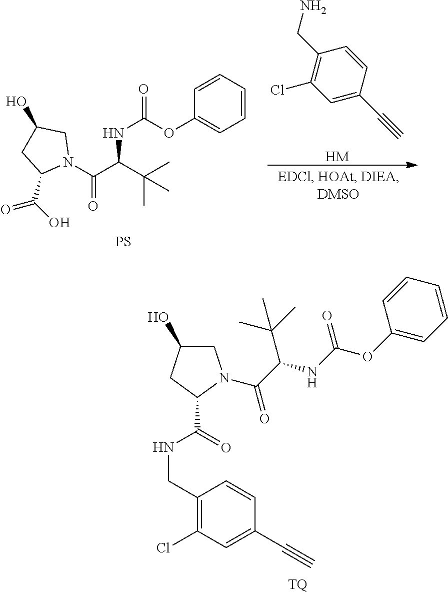

To a mixture of (2-chloro-4-ethynylphenyl)methanamine (500 mg, 2.47 mmol, Intermediate HM) and (2S,4R)-1-((S)-3,3-dimethyl-2-((phenoxycarbonyl)amino)butanoyl)-4-hydroxypyrrolidine-2-carboxylic acid (902 mg, 2.47 mmol, Intermediate PS) in DMSO (20 mL) was added EDCI (711 mg, 3.71 mmol), HOAT (505 mg, 3.71 mmol), and DIEA (959 mg, 7.42 mmol). The mixture was stirred at 25° C. for 2 hrs. On completion, the reaction mixture was diluted with H₂O (100 mL) and extracted with EA 300 mL (100 mL×3). The combined organic layers were dried over Na₂SO₄, filtered and concentrated under reduced pressure to give a residue. The residue was purified by column chromatography (SiO₂, Petroleum ether/Ethyl acetate=4/1 to 1/3) to give the title compound (1.2 g, 90% yield) as a white solid. LC-MS (ESI⁺) m/z 512.4 (M+H)⁺.

### 2-((tert-butoxycarbonyl)amino)-5-(2-ethoxy-2-oxoethoxy)-3,3-dimethylpentanoic acid (Intermediate TR)

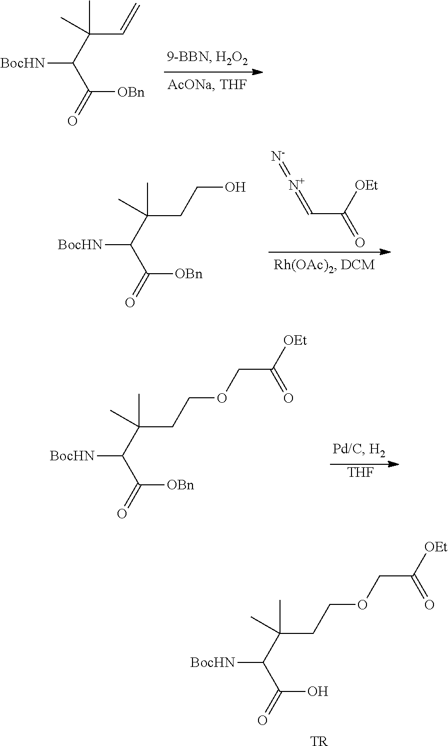

Step 1-Benzyl 2-((tert-butoxycarbonyl)amino)-5-hydroxy-3,3-dimethylpentanoate. To a solution of benzyl 2-(tert-butoxycarbonylamino)-3,3-dimethyl-pent-4-enoate (5 g, 15.0 mmol) in THF (50 mL) was added dropwise to 9-BBN (0.5 M, 75.0 mL) at 0° C. The mixture was then stirred at 25° C. for 1 hr. Then AcONa (4 M, 18.8 mL) and H₂O₂(17.0 g, 14.4 mL, 30% solution) was added dropwise at 0° C. The mixture was then stirred at 25° C. for 12 hrs. On completion, the reaction mixture was quenched with aq. NaHSO₃ mL at 0° C., and then extracted with EA (50 mL×3). The combined organic layers were washed with brine (50 mL), dried over Na₂SO₄, filtered and concentrated under reduced pressure to give a residue. The residue was purified by column chromatography (SiO₂, Petroleum ether/Ethyl acetate=20/1 to 3/1) to give title compound (3.4 g, 57% yield) as a colorless oil. LC-MS (ESI⁺) m/z 252.2 (M−99)⁺.

Step 2-Benzyl 2-((tert-butoxycarbonyl)amino)-5-(2-ethoxy-2-oxoethoxy)-3,3-dimethylpentanoate. To a solution of benzyl 2-(tert-butoxycarbonylamino)-5-hydroxy-3,3-dimethyl-pentanoate (1 g, 2.85 mmol) in DCM (10 mL) was added Rh(OAc)₂ (62.9 mg, 285 umol, CAS #42204-14-8) and se ethyl 2-diazoacetate (974 mg, 8.54 mmol, CAS #623-73-4) was added dropwise in DCM (10 mL) at 0° C. The mixture was stirred at 25° C. for 12 hrs. On completion, the reaction mixture was quenched with AcOH (3 mL) and H₂O (10 mL) at 0° C. and extracted with DCM (10 mL×3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=50/1 to 10/1) to give title compound (600 mg, 46% yield) as a yellow oil. LC-MS (ESI⁺) m/z 338.4 (M−99)⁺.

Step 3-2-((Tert-butoxycarbonyl)amino)-5-(2-ethoxy-2-oxoethoxy)-3,3-dimethylpentanoic acid. To a solution of benzyl 2-(tert-butoxycarbonylamino)-5-(2-ethoxy-2-oxo-ethoxy)-3,3-dimethyl-pentanoate (1.1 g, 2.51 mmol) in THF (20 mL) was added Pd/C (1.1 g, 10 wt %) under N₂ atmosphere. The suspension was degassed and purged with H₂ three times. The mixture was then stirred under H₂ (15 Psi) at 25° C. for 12 hrs. On completion, the reaction mixture was filtered with a kieselguhr then the filter cake was washed with DCM (20 mL). The filtrate was concentrated under reduced pressure to give the title compound (0.85 g) as a yellow oil. LC-MS (ESI⁺) m/z 248.1 (M−99)⁺.

### Ethyl 2-(((S)-4-((tert-butoxycarbonyl)amino)-5-((2S,4R)-2-((4-ethynylbenzyl)carbamoyl)-4-hydroxypyrrolidin-1-yl)-3,3-dimethyl-5-oxopentyl)oxy)acetate (Intermediate TS) and ethyl 2-(((R)-4-((tert-butoxycarbonyl)amino)-5-((2S,4R)-2-((4-ethynylbenzyl)carbamoyl)-4-hydroxypyrrolidin-1-yl)-3,3-dimethyl-5-oxopentyl)oxy)acetate (Intermediate TT)

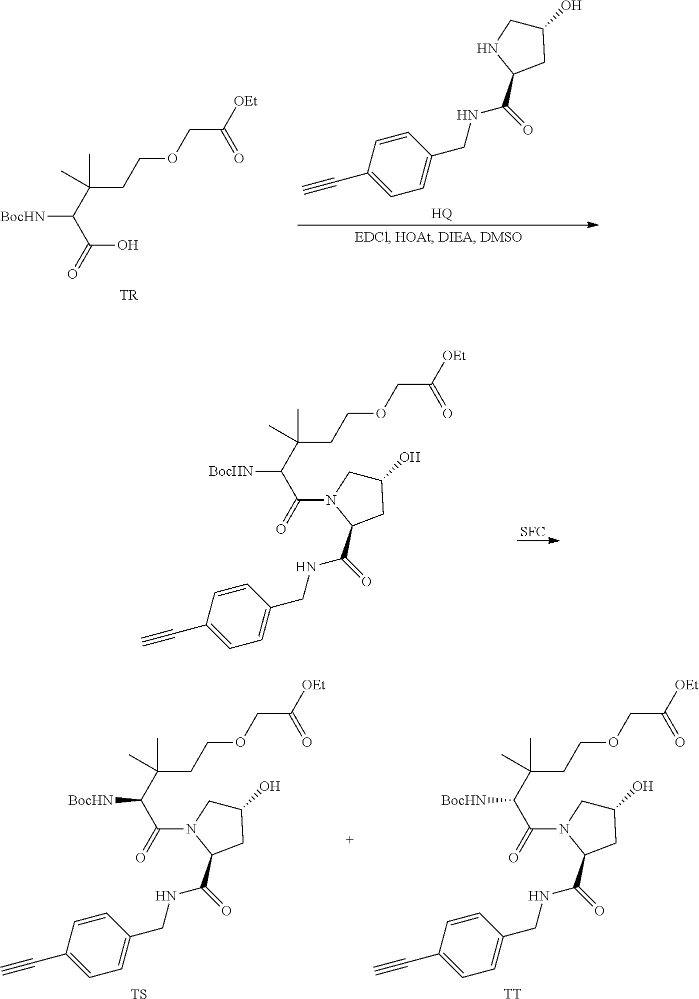

Step 1-Ethyl 2-((4-((tert-butoxycarbonyl)amino)-5-((2S,4R)-2-((4-ethynylbenzyl)carbamoyl)-4-hydroxypyrrolidin-1-yl)-3,3-dimethyl-5-oxopentyl)oxy)acetate. To a solution of 2-(tert-butoxycarbonylamino)-5-(2-ethoxy-2-oxo-ethoxy)-3,3-dimethyl-pentanoic acid (800 mg, 2.30 mmol, Intermediate TR) and (2S,4R)—N-[(4-ethynylphenyl)methyl]-4-hydroxy-pyrrolidine-2-carboxamide (450 mg, 1.84 mmol, Intermediate HQ) in DMSO (10 mL) was added EDCI (441 mg, 2.30 mmol), DIEA (893 mg, 6.91 mmol) and HOAt (313 mg, 2.30 mmol). The mixture was stirred at 25° C. for 12 hrs. On completion, the reaction mixture was quenched with H₂O (10 mL) and extracted with EA (10 mL×3). The combined organic layers were washed with brine (10 mL×3), dried over Na₂SO₄, filtered and concentrated under reduced pressure to give a residue. The crude product was purified by reversed-phase HPLC(0.1% FA condition) to give title compound (600 mg, 43% yield, FA salt) as a yellow oil. LC-MS (ESI⁺) m/z 474.3 (M−99)⁺.

Step 2-Ethyl 2-(((S)-4-((tert-butoxycarbonyl)amino)-5-((2S,4R)-2-((4-ethynylbenzyl)carbamoyl)-4-hydroxypyrrolidin-1-yl)-3,3-dimethyl-5-oxopentyl)oxy)acetate and ethyl 2-(((R)-4-((tert-butoxycarbonyl)amino)-5-((2S,4R)-2-((4-ethynylbenzyl)carbamoyl)-4-hydroxypyrrolidin-1-yl)-3,3-dimethyl-5-oxopentyl)oxy)acetate. To a solution of ethyl 2-[4-(tert-butoxycarbonylamino)-5-[(2S,4R)-2-[(4-ethynylphenyl)methylcarbamoyl]-4-hydroxy-pyrrolidin-1-yl]-3,3-dimethyl-5-oxo-pentoxy]acetate (640 mg, 1.12 mmol, FA salt) was purified by SFC (column: DAICEL CHIRALPAK AD(250 mm*30 mm,10 um);mobile phase: [0.1% NH₃H₂O IPA];B %: 30%-30%, C6.75; 102 min) to give ethyl 2-(((S)-4-((tert-butoxycarbonyl)amino)-5-((2S,4R)-2-((4-ethynylbenzyl)carbamoyl)-4-hydroxypyrrolidin-1-yl)-3,3-dimethyl-5-oxopentyl)oxy)acetate (215 mg, 32% yield) as a white solid (LC-MS (ESI⁺) m/z 474.5 (M−99)⁺) and ethyl 2-(((R)-4-((tert-butoxycarbonyl)amino)-5-((2S,4R)-2-((4-ethynylbenzyl)carbamoyl)-4-hydroxypyrrolidin-1-yl)-3,3-dimethyl-5-oxopentyl)oxy)acetate (235 mg, 37% yield) as a white solid (LC-MS (ESI⁺) m/z 474.5 (M−99)⁺). The absolute stereochemistry was assigned arbitrarily.

### Ethyl 2-(((S)-4-amino-5-((2S,4R)-2-((4-ethynylbenzyl)carbamoyl)-4-hydroxypyrrolidin-1-yl)-3,3-dimethyl-5-oxopentyl)oxy)acetate (Intermediate TU)

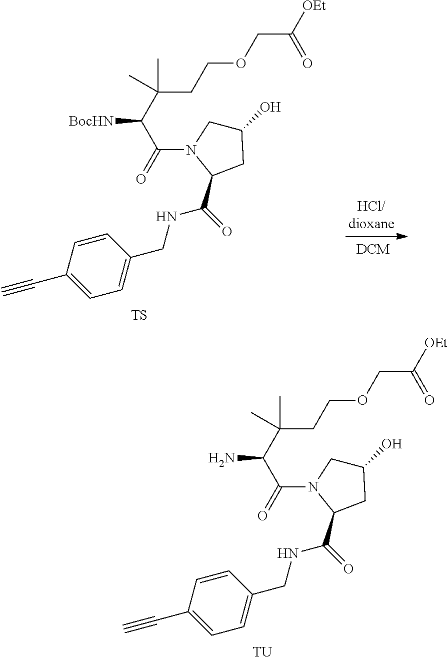

To a solution of ethyl 2-[(4S)-4-(tert-butoxycarbonylamino)-5-[(2S,4R)-2-[(4-ethynylphenyl)methylcarbamoyl]-4-hydroxy-pyrrolidin-1-yl]-3,3-dimethyl-5-oxo-pentoxy]acetate (215 mg, 375 umol, Intermediate TS) in DCM (2 mL) was added HCl/dioxane (4 M, 0.4 mL). The mixture was stirred at 25° C. for 8 hrs. On completion, the reaction mixture was diluted with DCM (20 mL) and concentrated under reduced pressure to give the title compound (210 mg, HCl salt) as a white solid. LC-MS (ESI⁺) m/z 474.3 (M+1)⁺.

### Ethyl 2-(((S)-5-((2S,4R)-2-((4-ethynylbenzyl)carbamoyl)-4-hydroxypyrrolidin-1-yl)-3,3-dimethyl-5-oxo-4-((phenoxycarbonyl)amino)pentyl)oxy)acetate (Intermediate TV)

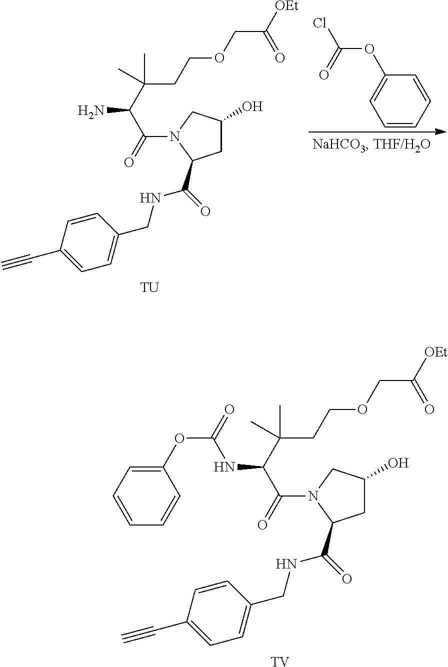

To a solution of ethyl 2-[(4S)-4-amino-5-[(2S,4R)-2-[(4-ethynylphenyl)methylcarbamoyl]-4-hydroxy-pyrrolidin-1-yl]-3,3-dimethyl-5-oxo-pentoxy]acetate (190 mg, 335 umol, HCl salt) in THF (3 mL) and H₂O (0.3 mL) was added NaHCO₃ (113 mg, 1.34 mmol) and phenyl carbonochloridate (63.0 mg, 402 umol). The mixture was stirred at 0° C. for 4 hrs. On completion, the reaction mixture was quenched with H₂O (5 mL) and extracted with DCM (5 mL×3). The combined organic layers were washed with brine (5 mL), dried over Na₂SO₄, filtered and concentrated under reduced pressure to give a residue. The residue was purified by column chromatography (SiO₂, Petroleum ether/Ethyl acetate=10/1 to 1/4) to give title compound (160 mg, 72% yield) as a white solid. LC-MS (ESI⁺) m/z594.3 (M+1)⁺.

### Ethyl 2-(((R)-4-amino-5-((2S,4R)-2-((4-ethynylbenzyl)carbamoyl)-4-hydroxypyrrolidin-1-yl)-3,3-dimethyl-5-oxopentyl)oxy)acetate (Intermediate TW)

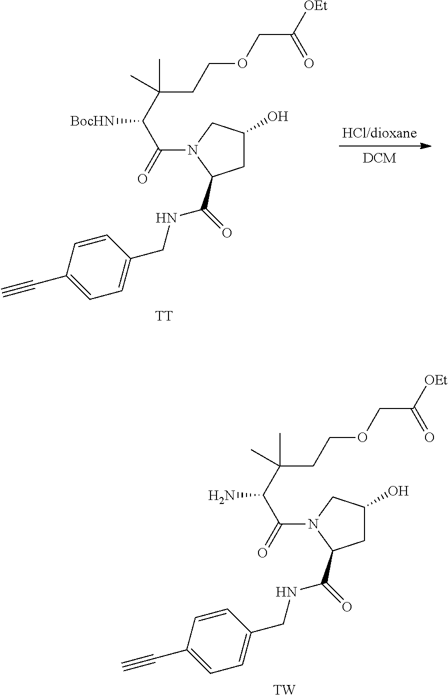

To a solution of ethyl 2-[(4R)-4-(tert-butoxycarbonylamino)-5-[(2S,4R)-2-[(4-ethynylphenyl)methylcarbamoyl]-4-hydroxy-pyrrolidin-1-yl]-3,3-dimethyl-5-oxo-pentoxy]acetate (215 mg, 375 umol, Intermediate TT) in DCM (2 mL) was added HCl/dioxane (4 M, 0.4 mL). The mixture was stirred at 25° C. for 2 hrs. On completion, the reaction mixture was diluted with DCM (20 mL) and concentrated under reduced pressure to give the title compound (211 mg, HCl salt), as a white solid. LC-MS (ESI⁺) m/z474.3 (M+1)⁺.

### Ethyl 2-(((R)-5-((2S,4R)-2-((4-ethynylbenzyl)carbamoyl)-4-hydroxypyrrolidin-1-yl)-3,3-dimethyl-5-oxo-4-((phenoxycarbonyl)amino)pentyl)oxy)acetate (Intermediate TX)

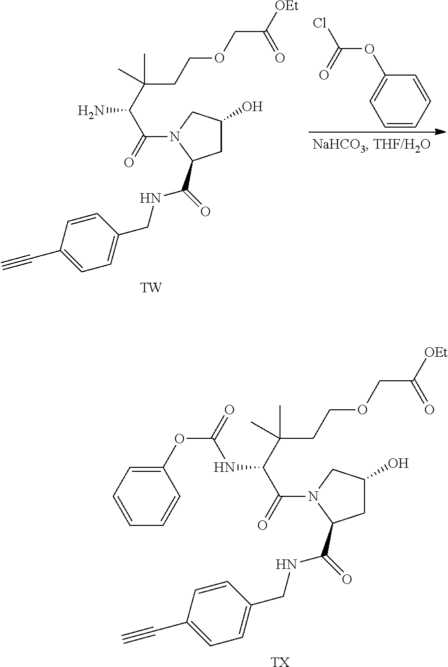

To a solution of ethyl 2-[(4R)-4-amino-5-[(2S,4R)-2-[(4-ethynylphenyl)methylcarbamoyl]-4-hydroxy-pyrrolidin-1-yl]-3,3-dimethyl-5-oxo-pentoxy]acetate (211 mg, 372 umol, HCl salt, Intermediate TW) in THF (3 mL) and H₂O (0.3 mL) was added NaHCO₃ (125 mg, 1.49 mmol) and phenyl carbonochloridate (70.0 mg, 447 umol). The mixture was stirred at 0° C. for 4 hrs. On completion, the reaction mixture was quenched with H₂O (5 mL) and extracted with DCM (5 mL×3). The combined organic layers were washed with brine (5 mL), dried over Na₂SO₄, filtered and concentrated under reduced pressure to give a residue. The residue was purified by column chromatography (SiO₂, Petroleum ether/Ethyl acetate=10/1 to 1/4) to give title compound (180 mg) as a white solid. LC-MS (ESI⁺) m/z 594.3 (M+1)⁺.

### (4-Ethynyl-3-methoxyphenyl)methanamine (Intermediate TY)

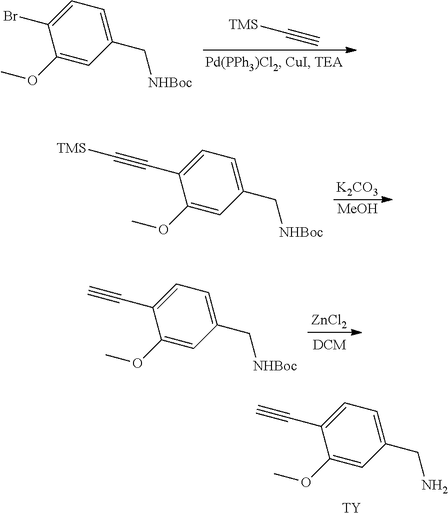

Step 1-Tert-butyl 3-methoxy-4-((trimethylsilyl)ethynyl)benzylcarbamate. To a solution of ethynyltrimethylsilane (1.55 g, 15.8 mmol, 2.19 mL) and tert-butyl 4-bromo-3-methoxybenzylcarbamate (500 mg, 1.58 mmol, synthesized via Steps 1-2 of Intermediate HO) was added Pd(PPh₃)₂Cl₂ (111 mg, 158 umol), CuI (60.2 mg, 316 umol) and TEA (7.27 g, 71.9 mmol, 10 mL) at 25° C., then the mixture was stirred at 80° C. for 12 hrs. On completion, the reaction mixture was filtered and concentrated in vacuo to give the crude residue. The residue was purified by column chromatography (SiO₂, Petroleum ether/Ethyl acetate=10/1 to 5/1) to give the title compound (7.5 g, 93% yield) as yellow oil. LC-MS (ESI⁺) m/z 278.2. (M−55)⁺.

Step 2-Tert-butyl 4-ethynyl-3-methoxybenzylcarbamate. To a solution of tert-butyl 3-methoxy-4-((trimethylsilyl)ethynyl)benzylcarbamate (200 mg, 560 umol) in MeOH (2 mL) was added K₂CO₃ (82.9 mg, 560 umol) at 25° C., then the mixture was stirred at 25° C. for 1 hr. On completion, the reaction mixture was quenched with water (5 mL) and extracted by ethyl acetate (10×3 mL). The extracts were washed by NH₄Cl (10 mL) and dried over anhydrous sodium sulfate, filtered and concentrated in vacuo to give the title compound (190 mg) as a yellow oil. LC-MS (ESI⁺) m/z 206.2. (M−55)⁺.

Step 3-(4-Ethynyl-3-methoxyphenyl)methanamine. To a solution of tert-butyl N-[(4-ethynyl-3-methoxy-phenyl)methyl]carbamate (50 mg, 191 umol) in DCM (1 mL) was added ZnCl₂ (104 mg, 765 umol, 35.8 uL) at 25° C., then the mixture was stirred at 25° C. for 48 hrs. On completion, the reaction mixture was filtered and concentrated in vacuo give the title compound (50 mg) as a yellow solid. LC-MS (ESI⁺) m/z 145.3. (M−16)⁺.

### Methyl 2-(5-bromopyrazin-2-yl)-2-azaspiro[3.3]heptane-6-carboxylate (Intermediate TZ)

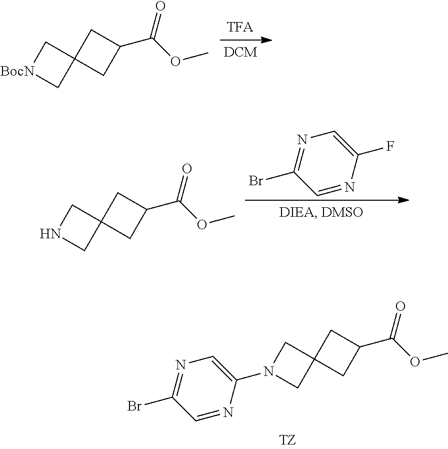

Step 1-Methyl 2-azaspiro[3.3]heptane-6-carboxylate. To a solution of 02-tert-butyl 06-methyl 2-azaspiro[3.3]heptane-2,6-dicarboxylate (1.5 g, 5.88 mmol, CAS #1408074-81-6) in DCM (15 mL) was added TFA (6.16 g, 54.0 mmol). The mixture was stirred at 25° C. for 1 hr. On completion, the reaction mixture was filtered and concentrated under reduced pressure to give the title compound (1.2 g, TFA) as a yellow oil. LC-MS (ESI⁺) m/z 156.1 (M+H)⁺.

Step 2-Methyl 2-(5-bromopyrazin-2-yl)-2-azaspiro[3.3]heptane-6-carboxylate. To a solution of methyl 2-azaspiro[3.3]heptane-6-carboxylate (1.2 g, 4.46 mmol, TFA), 2-bromo-5-fluoropyrazine (709 mg, 4.01 mmol, CAS #1209459-10-8) in DMSO (10 mL) was added DIEA (2.88 g, 22.2 mmol). The mixture was stirred at 60° C. for 1 hr. On completion, the reaction mixture was diluted with H₂O (90 mL) and extracted with EA (20 mL×3). The combined organic layers were washed with aqueous brine (5 mL×2), dried over Na₂SO₄, filtered and concentrated under reduced pressure to give a residue. The residue was purified by column chromatography (SiO₂, Petroleum ether/Ethyl acetate=10/1 to 4/1) to give the title compound (1.3 g, 89% yield) as a yellow oil. LC-MS (ESI⁺) m/z 312.0 (M+H)⁺.

### 2-(5-(4-oxopiperidin-1-yl)pyrazin-2-yl)-2-azaspiro[3.3]heptane-6-carboxylic acid (Intermediate UA)

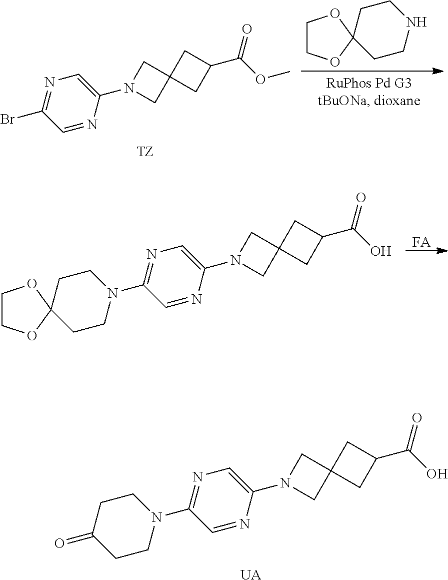

Step 1-2-(5-(1,4-Dioxa-8-azaspiro[4.5]decan-8-yl)pyrazin-2-yl)-2-azaspiro[3.3]heptane-6-carboxylic acid. To a solution of methyl 2-(5-bromopyrazin-2-yl)-2-azaspiro[3.3]heptane-6-carboxylate (1.1 g, 3.52 mmol, Intermediate TZ) in dioxane (20 mL) was added 1,4-dioxa-8-azaspiro[4.5]decane (1.01 g, 7.05 mmol, CAS #177-11-7), tBuONa (2 M, 7.05 mL) and RuPhos Pd G3 (294 mg, 352 umol). The mixture was stirred at 80° C. for 5 hrs under N₂ atmosphere. On completion, the reaction mixture was extracted with DCM (20 mL×3). The combined organic layers were washed with brine (20 mL×2), dried over Na₂SO₄, filtered and concentrated under reduced pressure to give a residue. The residue was purified by prep-HPLC (FA condition) to give the title compound (500 mg, 31% yield, FA) as a yellow solid. LC-MS (ESI⁺) m/z 361.1 (M+H)⁺.

Step 2-2-(5-(4-oxopiperidin-1-yl)pyrazin-2-yl)-2-azaspiro[3.3]heptane-6-carboxylic acid. A solution of 2-[5-(1,4-dioxa-8-azaspiro[4.5]decan-8-yl)pyrazin-2-yl]-2-azaspiro[3.3]heptane-6-carboxylic acid (400 mg, 1.11 mmol) in FA (4 mL) was stirred at 25° C. for 1 hr. On completion, the reaction mixture was filtered and concentrated under reduced pressure to give a residue. The residue was purified by prep-HPLC (FA condition) to give the title compound (300 mg, 64% yield, FA) as a brown solid. LC-MS (ESI⁺) m/z 317.1 (M+H)⁺.

### (S)-2-(5-(4-(2-(2-hydroxyphenyl)-6a,7,9,10-tetrahydro-5H-pyrazino[1′,2′:4,5]pyrazino[2,3-c]pyridazin-8(6H)-yl)piperidin-1-yl)pyrazin-2-yl)-2-azaspiro[3.3]heptane-6-carboxylic acid (Intermediate UB)

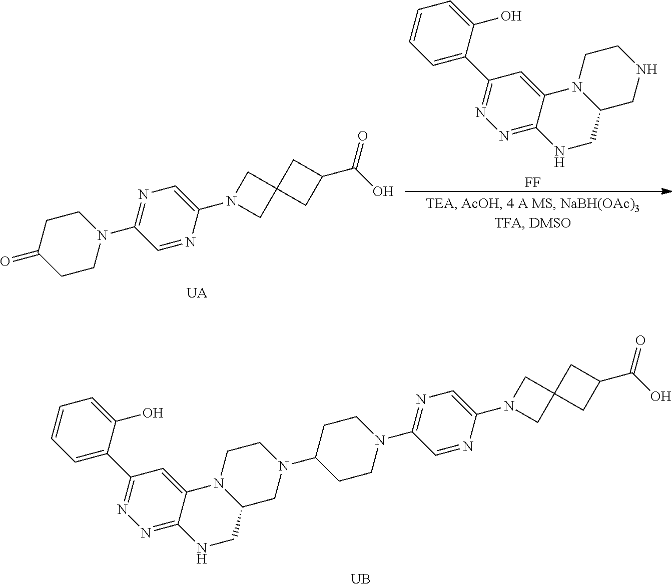

To a solution of 2-[(1OR)-1,5,6,8,12-pentazatricyclo[8.4.0.0^{2,7}]tetradeca-2,4,6-trien-4-yl]phenol (150 mg, 469 umol, HCl, Intermediate FF) in THF (2 mL) and DMSO (2 mL) was added TEA (142 mg, 1.41 mmol) and 4 Å molecular sieves (469 umol) and the mixture was stirred 0.5 hr. Then 2-[5-(4-oxo-1-piperidyl)pyrazin-2-yl]-2-azaspiro[3.3]heptane-6-carboxylic acid (192 mg, 609 umol, Intermediate UA) and AcOH (84.5 mg, 1.41 mmol) was added and the mixture was stirred for 12 hrs at 40° C. Then NaBH(OAc)₃ (298 mg, 1.41 mmol) was added at 0° C. then the mixture was stirred at 25° C. for 1.5 hrs. On completion, the reaction mixture was quenched with MeOH (1 mL) at 25° C., and then filtered and concentrated under reduced pressure to give a residue. The residue was purified by reversed-phase HPLC (FA) to give the title compound (35 mg, 12% yield, FA) as a yellow solid. LC-MS (ESI⁺) m/z 584.2 (M+H)⁺.

### Tert-butyl 4-(1-bromo-2-oxoethyl)piperidine-1-carboxylate (Intermediate UD)

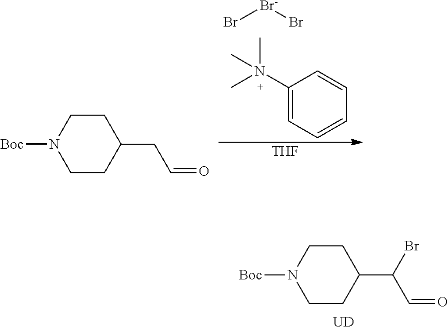

A mixture of tert-butyl 4-(2-oxoethyl)piperidine-1-carboxylate (9 g, 39.60 mmol, CAS #142374-19-4), trimethylphenylammonium tribromide (22.33 g, 59.39 mmol, CAS #4207-56-4) in THF (100 mL) was degassed and purged with N₂ three times. Then the mixture was stirred at 0° C. for 1 hr under N₂ atmosphere. The reaction mixture was partitioned between ethyl acetate (200 mL) and water (180 mL). The organic phase was separated, washed with brine (90 mL×2), dried over Na₂SO₄, filtered and concentrated under reduced pressure to give the title compound (12 g) as a brown oil.

### (R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)thiazol-5-yl)piperidine-1-carboxylate (Intermediate UE) and (S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)thiazol-5-yl)piperidine-1-carboxylate (Intermediate UF)

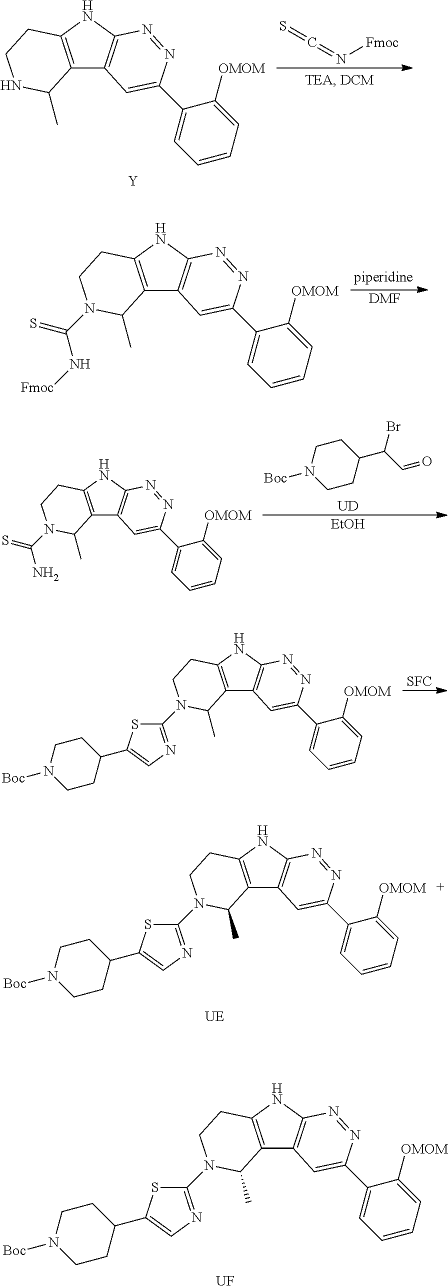

Step 1-(9H-fluoren-9-yl)methyl (3-(2-(methoxymethoxy)phenyl)-5-methyl-6,7,8,9-tetrahydro-5H-pyrido[3′,4′:4,5]pyrrolo[2,3-c]pyridazine-6-carbonothioyl)carbamate. To a solution of 12-[2-(methoxymethoxy)phenyl]-3-methyl-4,8,10,11-tetrazatricyclo[7.4.0.0^{2,7}]trideca-1(9),2(7),10,12-tetraene (4 g, 12.3 mmol, Intermediate Y) in DCM (80 mL) was added TEA (1.25 g, 12.3 mmol) and 9H-fluoren-9-ylmethyl N-(thioxomethylene)carbamate (3.82 g, 13.6 mmol, CAS #199915-38-3). The mixture was stirred at 0-25° C. for 1 hr. On completion, the reaction mixture was filtered and concentrated under reduced pressure to give a residue. The crude product was triturated with Petroleum ether/Ethyl acetate=1/1 at 25° C. for 30 min, then filterd and dried to give the title compound (6.2 g, 58% yield) as a white solid. LC-MS (ESI⁺) m/z 606.4 (M+H)⁺.

Step 2-3-(2-(Methoxymethoxy)phenyl)-5-methyl-7,8-dihydro-5H-pyrido[3′,4′:4,5]pyrrolo[2,3-c]pyridazine-6(9H)-carbothioamide. To a solution of 9H-fluoren-9-ylmethyl N-[12-[2-(methoxymethoxy)phenyl]-3-methyl-4,8,10,11-tetrazatricyclo[7.4.0.0^{2,7}]trideca-1(9),2(7),10,12-tetraene-4-carbothioyl]carbamate (5 g, 8.25 mmol) was added in DMF (25 mL) and piperidine (25 mL). The mixture was stirred at 25° C. for 1 hr. On completion, the reaction mixture was filtered and concentrated under reduced pressure to give a residue. The crude product was purified by reversed-phase HPLC (0.1% FA condition) to give the title compound (3.7 g, 89% yield) as a yellow solid. LC-MS (ESI+) m/z 384.1 (M+H)⁺.

Step 3-Tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)thiazol-5-yl)piperidine-1-carboxylate. A mixture of tert-butyl 4-(1-bromo-2-oxo-ethyl)piperidine-1-carboxylate (11.4 g, 37.3 mmol, Intermediate UD), 12-[2-(methoxymethoxy)phenyl]-3-methyl-4,8,10,11-tetrazatricyclo[7.4.0.0^{2,7}]trideca-1(9),2(7),10,12-tetraene-4-carbothioamide (2 g, 4.66 mmol) in EtOH (100 mL) was degassed and purged with N₂ three times. Then the mixture was stirred at 80° C. for 5 hrs under N₂ atmosphere. On completion, the reaction mixture was filtered and concentrated under reduced pressure to give a residue. The crude product was purified by reversed-phase HPLC (0.1% FA condition) to give the title compound (600 mg, 18% yield) as a yellow solid. LC-MS (ESI⁺) m/z 547.4 (M−43)⁺.

Step 4-(R)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)thiazol-5-yl)piperidine-1-carboxylate and (S)-tert-butyl 4-(2-(3-(2-(methoxymethoxy)phenyl)-5-methyl-7,8-dihydro-5H-pyrido[3′,4′:4,5]pyrrolo[2,3-c]pyridazin-6(9H)-yl)thiazol-5-yl)piperidine-1-carboxylate. Tert-butyl 4-[2-[12-[2-(methoxymethoxy)phenyl]-3-methyl-4,8,10,11-tetrazatricyclo[7.4.0.0^{2,7}]trideca-1(9),2(7),10,12-tetraen-4-yl]thiazol-5-yl]piperidine-1-carboxylate (500 mg, 846 umol) was further separated by SFC (column: DAICEL CHIRALPAK AS(250 mm×30 mm, 10 um); mobile phase: [0.1% NH₃H₂O MEOH]; B %: 50%-50%, A5.9; 61 min) to give tert-butyl 4-[2-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]thiazol-5-yl]piperidine-1-carboxylate (120 mg, 23% yield) as a yellow solid (LC-MS (ESI+) m/z 547.2 (M−43)⁺) and tert-butyl 4-[2-[(3S)-12-[2-(methoxymethoxy)phenyl]-3-methyl-4,8,10,11-tetrazatricyclo[7.4.0.0^{2,7}]trideca-1(9),2(7),10,12-tetraen-4-yl]thiazol-5-yl]piperidine-1-carboxylate (110 mg, 22% yield) as a white solid (LC-MS (ESI+) m/z 547.4 (M−43)⁺). Absolute stereochemistry of the enantiomers was assigned arbitrarily.

### (R)-2-(5-methyl-6-(5-(piperidin-4-yl)thiazol-2-yl)-6,7,8,9-tetrahydro-5H-pyrido[3′,4′:4,5]pyrrolo[2,3-c]pyridazin-3-yl)phenol (Intermediate UG)

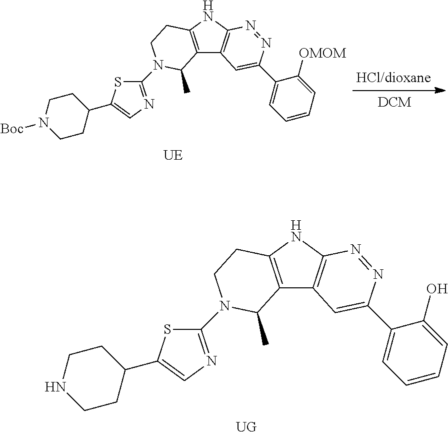

To a solution of tert-butyl 4-[2-[(3R)-12-[2-(methoxymethoxy)phenyl]-3-methyl-4,8,10,11-tetrazatricyclo[7.4.0.0^{2,7}]trideca-1(9),2(7),10,12-tetraen-4-yl]thiazol-5-yl]piperidine-1-carboxylate (120 mg, 203 umol, Intermediate UE) was added in DCM (2 mL) and HCl/dioxane (0.5 mL). The mixture was stirred at 25° C. for 1 hr. On completion, the reaction mixture was filtered and concentrated under reduced pressure to give the title compound (120 mg) as a yellow solid. LC-MS (ESI+) m/z 447.4 (M+H)⁺.

### (R)-2-(6-(5-(1-(2-azaspiro[3.3]heptan-6-yl)piperidin-4-yl)thiazol-2-yl)-5-methyl-6,7,8,9-tetrahydro-5H-pyrido[3′,4′:4,5]pyrrolo[2,3-c]pyridazin-3-yl)phenol (Intermediate UH)

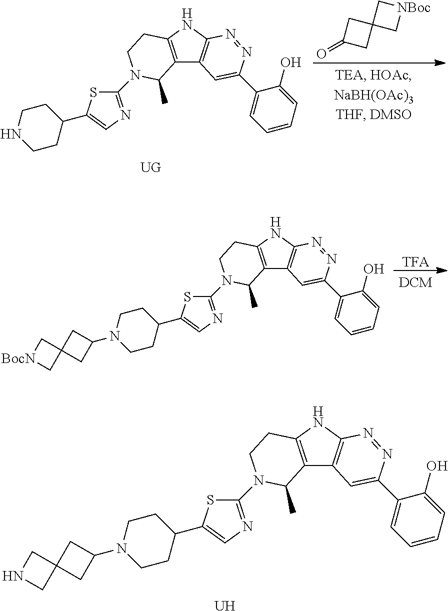

Step 1-(R)-tert-butyl 6-(4-(2-(3-(2-hydroxyphenyl)-5-methyl-7,8-dihydro-5H-pyrido[3′,4′:4,5]pyrrolo[2,3-c]pyridazin-6(9H)-yl)thiazol-5-yl)piperidin-1-yl)-2-azaspiro[3.3]heptane-2-carboxylate. To a solution of 2-[(3R)-3-methyl-4-[5-(4-piperidyl)thiazol-2-yl]-4,8,10,11-tetrazatricyclo[7.4.0.0^{2,7}]trideca-1(9),2(7),10,12-tetraen-12-yl]phenol (120 mg, 248 umol, Intermediate UG) in THF (1 mL) and DMSO (0.2 mL) was added TEA (50.3 mg, 497 umol) and the mixture was stirred at 25° C. for 30 min. Then, tert-butyl 6-oxo-2-azaspiro[3.3]heptane-2-carboxylate (105 mg, 497 umol, CAS #1181816-12-5) and HOAc (37.3 mg, 621 umol) was added and the mixture was stirred at 25° C. for 30 mi. Finally, NaBH(OAc)₃ (132 mg, 621 umol) was added and the mixture was stirred at 0-25° C. for 3 hrs. On completion, the reaction mixture was filtered and concentrated under reduced pressure to give a residue. The crude product was purified by reversed-phase HPLC (0.1% FA condition) to give the title compound (130 mg, 75% yield) as a yellow solid. ¹H NMR (400 MHz, DMSO-d₆) δ=12.49 (s, 1H), 8.71 (s, 1H), 8.18-8.14 (m, 1H), 7.35-7.25 (m, 1H), 7.03-6.89 (m, 2H), 6.87 (s, 1H), 5.36 (q, J=6.4 Hz, 1H), 4.18 (s, 1H), 4.00-3.86 (m, 1H), 3.84-3.77 (m, 2H), 3.72 (s, 2H), 3.66-3.60 (m, 1H), 3.16-3.03 (m, 2H), 2.89 (dd, J=3.6, 17.2 Hz, 1H), 2.80 (d, J=11.2 Hz, 1H), 2.60 (dd, J=8.0, 16.0 Hz, 1H), 2.37 (ddd, J=3.2, 6.8, 9.6 Hz, 2H), 2.27-2.21 (m, 2H), 1.96-1.80 (m, 7H), 1.56 (d, J=6.4 Hz, 3H), 1.52-1.42 (m, 2H), 1.35 (d, J=2.0 Hz, 9H). LC-MS (ESI+) m/z 642.3 (M+H)⁺.

Step 2-(R)-2-(6-(5-(1-(2-azaspiro[3.3]heptan-6-yl)piperidin-4-yl)thiazol-2-yl)-5-methyl-6,7,8,9-tetrahydro-5H-pyrido[3′,4′:4,5]pyrrolo[2,3-c]pyridazin-3-yl)phenol. To a solution of tert-butyl 6-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]thiazol-5-yl]-1-piperidyl]-2-azaspiro[3.3]heptane-2-carboxylate (130 mg, 203 umol) was added in TFA (0.1 mL) and DCM (0.5 mL). The mixture was stirred at 25° C. for 1 hr. On completion, the reaction mixture was filtered and concentrated under reduced pressure to give the title compound (130 mg) as a yellow solid. LC-MS (ESI+) m/z 542.5 (M+H)⁺.

### Ethyl 2-(3-(4-formylpiperidin-1-yl)isoxazol-5-yl)-3-methylbutanoate (Intermediate UI)

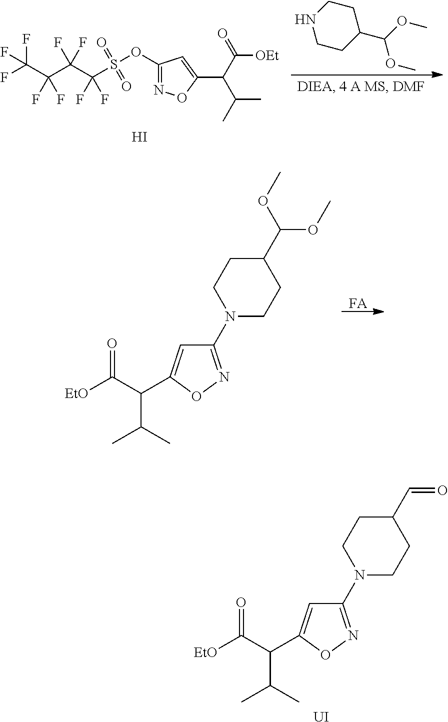

Step 1-Ethyl 2-(3-(4-(dimethoxymethyl)piperidin-1-yl)isoxazol-5-yl)-3-methylbutanoate. To a solution of ethyl 3-methyl-2-[3-(1,1,2,2,3,3,4,4,4-nonafluorobutylsulfonyloxy)isoxazol-5-yl]butanoate (6 g, 12.1 mmol, Intermediate IH) and 4-(dimethoxymethyl)piperidine (2.89 g, 18.2 mmol, CAS #188646-83-5) in DMF (50 mL) was added DIEA (4.70 g, 36.3 mmol) and 4 Å molecular sieves (5 g, 12 mmol). The mixture was stirred at 130° C. for 1 hr. On completion, the reaction mixture was partitioned between ethyl acetate (200 mL) and water (160 mL). The organic phase was separated, washed with brine (80 mL×2), dried over Na₂SO₄, filtered and concentrated under reduced pressure to give a residue. The residue was purified by column chromatography (SiO₂, Petroleum ether/Ethyl acetate=20/1 to 1/1) to give the title compound (3.8 g, 83% yield) as a yellow oil. LC-MS (ESI+) m/z 355.9 (M+H)⁺.

Step 2-Ethyl 2-(3-(4-formylpiperidin-1-yl)isoxazol-5-yl)-3-methylbutanoate. To a solution of ethyl 2-[3-[4-(dimethoxymethyl)-1-piperidyl]isoxazol-5-yl]-3-methyl-butanoate (2 g, 5.64 mmol) was added in methanoic acid (1 mL). The mixture was stirred at 25° C. for 1 hr. On completion, the reaction mixture was filtered and concentrated under reduced pressure to give the title compound (2 g) as a yellow oil. LC-MS (ESI+) m/z 327.2 (M+19)⁺.

### 2-(3-(4-((4-((S)-2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)methyl)piperidin-1-yl)isoxazol-5-yl)-3-methylbutanoic acid (Intermediate UJ)

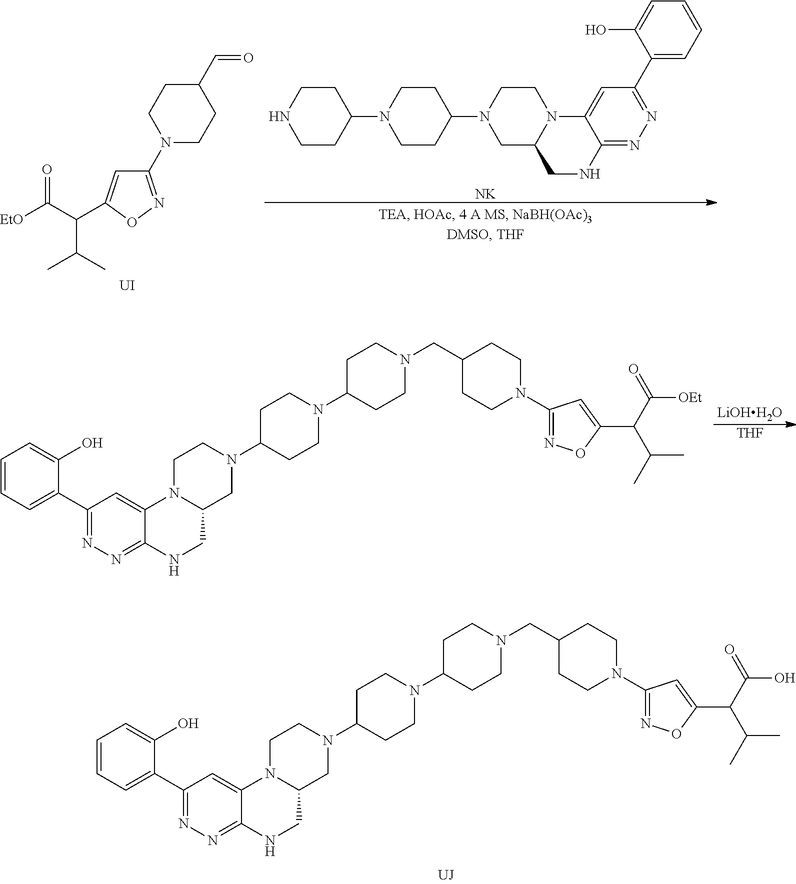

Step 1-Ethyl 2-(3-(4-((4-((S)-2-(2-hydroxyphenyl)-6a,7,9,10-tetrahydro-5H-pyrazino[1′,2′: 4,5]pyrazino[2,3-c]pyridazin-8(6H)-yl)-[1,4′-bipiperidin]-1′-yl)methyl)piperidin-1-yl)isoxazol-5-yl)-3-methylbutanoate. To a solution of 2-[(1OS)-12-[1-(4-piperidyl)-4-piperidyl]-1,5,6,8,12-pentazatricyclo[8.4.0.0^{2,7}]tetradeca-2,4,6-trien-4-yl]phenol (2.5 g, 5.56 mmol, Intermediate NK) in THF (20 mL) and DMSO (5 mL) was added TEA (1.13 g, 11.1 mmol) and was stirred at 25° C. for 30 min. Then, ethyl 2-[3-(4-formyl-1-piperidyl)isoxazol-5-yl]-3-methyl-butanoate (1.97 g, 5.56 mmol, Intermediate UI) and HOAc (668 mg, 11.1 mmol) was added and the mixture was stirred at 25° C. for 30 min. Finally, NaBH(OAc)₃ (2.95 g, 13.9 mmol) was added and the mixture was stirred at 0-25° C. for 12 hrs. On completion, the reaction mixture was filtered and concentrated under reduced pressure to give a residue. The crude product was purified by reversed-phase HPLC (0.1% FA condition) to give the title compound (1.2 g, 18% yield) as a yellow solid. LC-MS (ESI+) m/z 742.4 (M+H)⁺.

Step 2-2-(3-(4-((4-((S)-2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)methyl)piperidin-1-yl)isoxazol-5-yl)-3-methylbutanoic acid. To a solution of ethyl 2-[3-[4-[[4-[4-[(10S)-4-(2-hydroxyphenyl)-1,5,6,8,12-pentazatricyclo[8.4.0.0^{2,7}]tetradeca-2,4,6-trien-12-yl]-1-piperidyl]-1-piperidyl]methyl]-1-piperidyl]isoxazol-5-yl]-3-methyl-butanoate (1 g, 1.35 mmol) in THF (5 mL) was added LiOH·H₂O (2 M, 5.00 mL). The mixture was stirred at 25° C. for 1 hr. On completion, the reaction mixture was filtered and concentrated under reduced pressure to give a residue. The crude product was purified by reversed-phase HPLC (0.1% HCl condition) to give the title compound (700 mg, 53% yield) as a yellow solid. LC-MS (ESI+) m/z 714.5 (M+H)⁺.

### 1-(2-Chloro-4-ethynylphenyl)ethanamine (Intermediate UK)

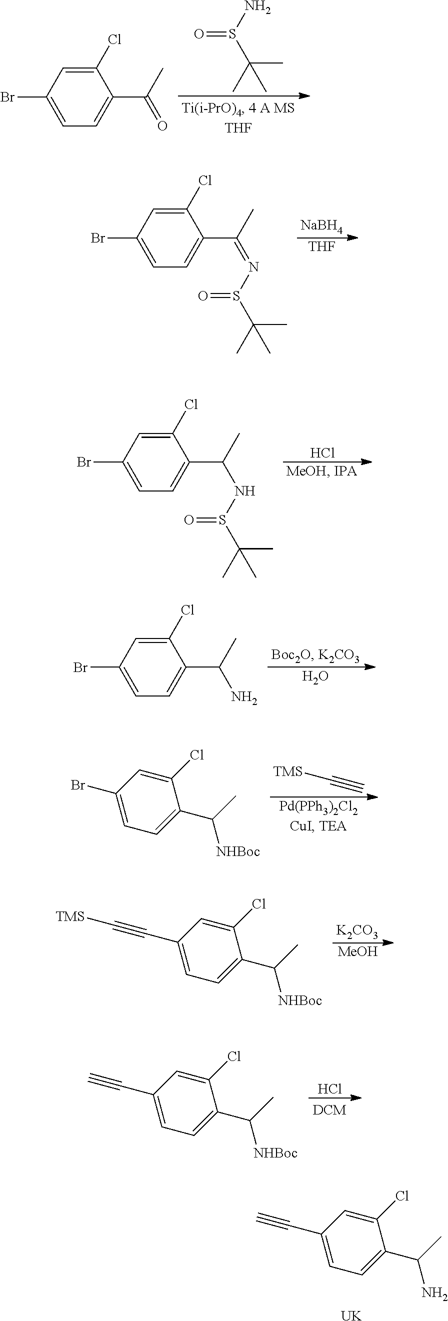

Step 1-(Z)—N-(1-(4-bromo-2-chlorophenyl)ethylidene)-2-methylpropane-2-sulfinamide. To a solution of 1-(4-bromo-2-chloro-phenyl)ethanone (20 g, 85.7 mmol, CAS #252561-81-2) 2-methylpropane-2-sulfinamide (51.9 g, 428 mmol) in THF (500 mL) was added Ti(i-PrO)₄ (122 g, 428 mmol) at 0° C. The mixture was stirred at 80° C. for 12 hrs. On completion, the reaction mixture was filtered to give the solution and then diluted with H₂O (100 mL) and extracted with EA (200 mL×3). The combined organic layers were washed with sat. NaCl (100 mL×1), dried over Na₂SO₄, filtered and concentrated under reduced pressure to give a residue. The mixture was purified by MPLC(SiO₂, PE:EA=10:1 to 5:1) to give the title compound (12 g, 40% yield) as a white solid. LC-MS (ESI⁺) m/z 337.8 (M+H)⁺.

Step 2-N-(1-(4-bromo-2-chlorophenyl)ethyl)-2-methylpropane-2-sulfinamide. A mixture of (NZ)—N-[1-(4-bromo-2-chloro-phenyl)ethylidene]-2-methyl-propane-2-sulfinamide (5 g, 14.8 mmol), NaBH₄ (1.69 g, 44.5 mmol) in THF (50 mL) at 0° C. was degassed and purged with N₂ three times. Then the mixture was stirred at 25° C. for 3 hrs under N₂ atmosphere. On completion, the mixture was quenched by H₂O (10 mL) at 0° C., and then extracted by EA (20 mL×3). The combined organic layer was washed with sat. NaCl (20 mL×1) and dried over Na₂SO₄, the mixture was concentrated under reduce pressure to give the title compound (5 g) as a white solid. LC-MS (ESI⁺) m/z 234.9(M+H)⁺.

Step 3-1-(4-Bromo-2-chlorophenyl)ethanamine. To a solution of tert-butyl N-[1-(4-bromo-2-chloro-phenyl)ethyl]-2-methyl-propane-2-sulfinamide (2 g, 5.91 mmol) in MeOH (16 mL) was added HCl (12 M, 4 mL) solution in IPA (4 mL). The mixture was stirred at 25° C. for 1 hr. On completion, the mixture was concentrated under reduce pressure to give the title compound (1.5 g) as a white solid. LC-MS (ESI+) m/z218.9 (M+H)⁺.

Step 4-Tert-butyl (1-(4-bromo-2-chlorophenyl)ethyl)carbamate. To a solution of 1-(4-bromo-2-chloro-phenyl)ethanamine (1.4 g, 5.97 mmol) in H₂O (12 mL) was added K₂CO₃ (4.95 g, 35.8 mmol) and Boc₂O (1.45 g, 6.63 mmol, 1.52 mL). The mixture was stirred at 25° C. for 12 hrs. On completion, the reaction mixture was quenched with sat. NH₄Cl (10 mL) at 25° C., and then extracted with EA (20 mL×3). The combined organic layers were washed with sat NaCl (30 mL), dried over Na₂SO₄, filtered and concentrated under reduced pressure to give a residue. The crude product was purified by MPLC (SiO₂, PE:EA=10:1 to 5:1) to give the title compound (1 g, 45% yield) as a yellow oil. LC-MS (ESI⁺) m/z279.8 (M+H)⁺.

Step 5-Tert-butyl (1-(2-chloro-4-((trimethylsilyl)ethynyl)phenyl)ethyl)carbamate. A mixture of tert-butyl N-[1-(4-bromo-2-chloro-phenyl)ethyl]carbamate (2 g, 6 mmol), ethynyl(trimethyl)silane (5.87 g, 59.8 mmol), Pd(PPh₃)₂Cl₂ (210 mg, 299 umol), CuI (114 mg, 598 umol) in TEA (25 mL) was degassed and purged with N₂ three times. Then the mixture was stirred at 80° C. for 12 hrs under N₂ atmosphere. The reaction mixture was diluted with EA (90 mL) and extracted with H₂O (20 mL×3). The combined organic layers were washed with NaCl (20 mL×3), dried over Na₂SO₄, and concentrated under reduced pressure to give a residue. The residue was purified by column chromatography (SiO₂, Petroleum ether/Ethyl acetate=50/1 to 10/1) to give the title compound (2.1 g, 78% yield) as a white solid.

Step 6-Tert-butyl (1-(2-chloro-4-ethynylphenyl)ethyl)carbamate. To a solution of tert-butyl N-[1-[2-chloro-4-(2-trimethylsilylethynyl)phenyl]ethyl]carbamate (1.9 g, 5.40 mmol) in MeOH (15 mL) was added K₂CO₃ (1.49 g, 10.8 mmol). The mixture was then stirred at 25° C. for 1 hr. On completion, the reaction mixture was diluted with H₂O (30 mL) and extracted with EA (20 mL×3). The combined organic layers were washed with NaCl (10 mL×3), dried over Na₂SO₄, filtered and concentrated under reduced pressure to give a residue. The residue was purified by column chromatography (SiO₂, Petroleum ether/Ethyl acetate=50/1 to 10/1) to give the title compound (1.4 g, 74% yield) as a white solid. LC-MS (ESI⁺) m/z163.4 (M−116)⁺.

Step 7-1-(2-chloro-4-ethynylphenyl)ethanamine. To a solution of tert-butyl N-[1-(2-chloro-4-ethynyl-phenyl)ethyl]carbamate (1.3 g, 4.65 mmol,) in DCM (13 mL) was added HCl/dioxane (4 M, 5 mL). The mixture was stirred at 25° C. for 1 hr. On completion, the reaction mixture was concentrated under reduced pressure to give the title compound (800 mg, HCl) as a white solid. LC-MS (ESI⁺) m/z163.1 (M+H)⁺.

### Tert-butyl (2S,4R)-2-[[(1R)-1-(2-chloro-4-ethynyl-phenyl)ethyl]carbamoyl]-4-hydroxy-pyrrolidine-1-carboxylate (Intermediate UL) and tert-butyl (2S,4R)-2-[[(1S)-1-(2-chloro-4-ethynyl-phenyl)ethyl]carbamoyl]-4-hydroxy-pyrrolidine-1-carboxylate (Intermediate UM)

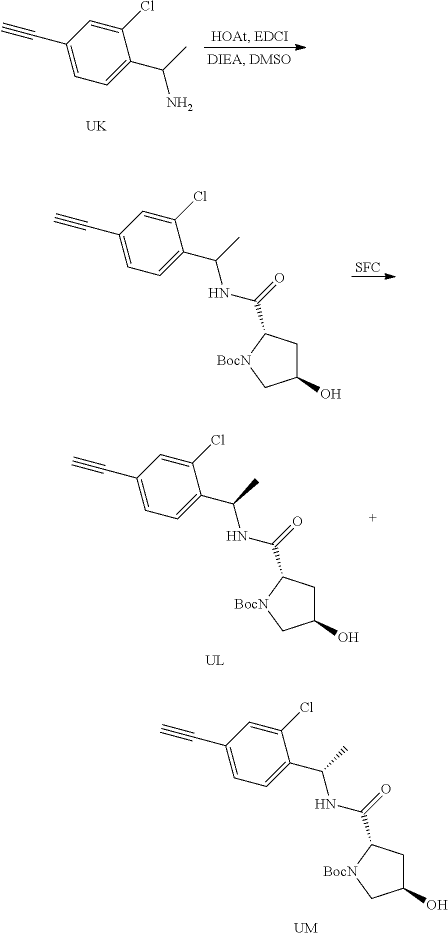

Step 1-(2S,4R)—N-(1-(2-chloro-4-ethynylphenyl)ethyl)-4-hydroxypyrrolidine-2-carboxamide. To a solution of (2S,4R)-1-tert-butoxycarbonyl-4-hydroxy-pyrrolidine-2-carboxylic acid (837 mg, 3.62 mmol) in DMSO was added EDCI (832 mg, 4.34 mmol), DIEA (1.40 g, 10.9 mmol, 1.89 mL) and HOAt (591 mg, 4.34 mmol, 6078 uL). After 10 mins, 1-(2-chloro-4-ethynyl-phenyl)ethanamine (650 mg, 3.62 mmol, Intermediate UK) was added and the mixture was stirred at 25° C. for 12 hrs. The crude product was purified by reversed-phase HPLC (0.1% FA condition) to give the title compound (550 mg, 33% yield, FA) as a white solid. LC-MS (ESI⁺) m/z293.1 (M+H−Boc)⁺.

Step 2-(2S,4R)—N—((S)-1-(2-chloro-4-ethynylphenyl)ethyl)-4-hydroxypyrrolidine-2-carboxamide and (2S,4R)—N—((R)-1-(2-chloro-4-ethynylphenyl)ethyl)-4-hydroxypyrrolidine-2-carboxamide. Compound tert-butyl (2S,4R)-2-[1-(2-chloro-4-ethynyl-phenyl)ethylcarbamoyl]-4-hydroxy-pyrrolidine-1-carboxylate (550 mg, 1.40 mmol) was purified by SFC (column: REGIS (R,R)WHELK-01 (250 mm×25 mm, 10 um);mobile phase: 0.1% NH₃H₂O MEOH;B %: 30%-30%) to give tert-butyl (2S,4R)-2-[[(1R)-1-(2-chloro-4-ethynyl-phenyl)ethyl]carbamoyl]-4-hydroxy-pyrrolidine-1-carboxylate (200 mg, 34% yield) and tert-butyl (2S,4R)-2-[[(1S)-1-(2-chloro-4-ethynyl-phenyl)ethyl]carbamoyl]-4-hydroxy-pyrrolidine-1-carboxylate (200 mg, 34% yield) as a white solids. LC-MS (ESI⁺) m/z 293.1 (M+H)⁺ for both isomers.

### (2S,4R)—N—((R)-1-(2-chloro-4-ethynylphenyl)ethyl)-4-hydroxypyrrolidine-2-carboxamide (Intermediate UN)

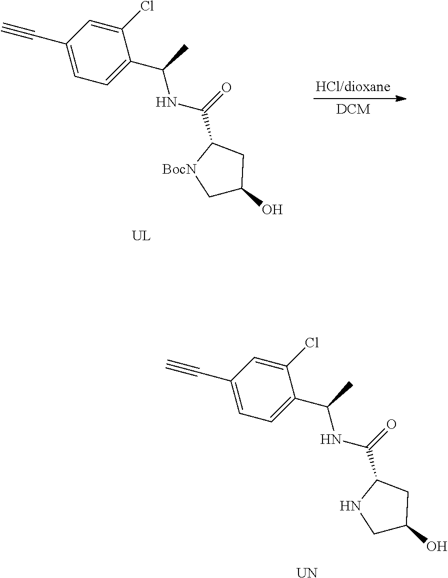 [SMILES: C#Cc1ccc([C@@H](C)NC(=O)[C@@H]2C[C@@H](O)CN2C(=O)OC(C)(C)C)c(Cl)c1 ; C#Cc1ccc([C@@H](C)NC(=O)[C@@H]2C[C@@H](O)CN2)c(Cl)c1 ; "UN"]

To a solution of tert-butyl (2S,4R)-2-[[(1R)-1-(2-chloro-4-ethynyl-phenyl)ethyl]carbamoyl]-4-hydroxy-pyrrolidine-1-carboxylate (50.00 mg, 127 umol, Intermediate UL) in DCM (1 mL) was added HCl/dioxane (4 M, 0.25 mL). The mixture was stirred at 25° C. for 1 hr. On completion, the reaction mixture was concentrated under reduced pressure to give the title compound (50 mg, HCl) as a white solid. LC-MS (ESI⁺) m/z 293.0 (M+H)⁺.

### (2S,4R)—N—((S)-1-(2-chloro-4-ethynylphenyl)ethyl)-4-hydroxypyrrolidine-2-carboxamide (Intermediate UO)

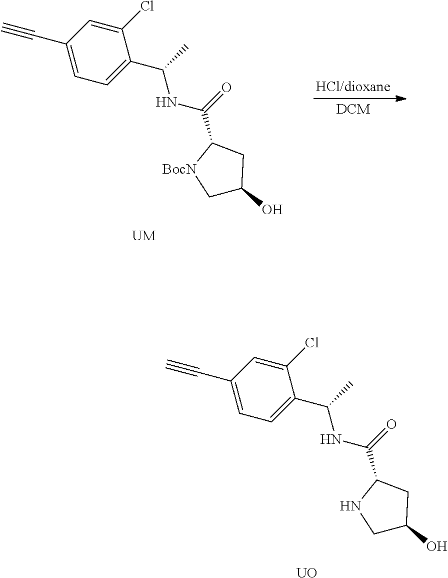

To a solution of tert-butyl (2S,4R)-2-[[(1S)-1-(2-chloro-4-ethynyl-phenyl)ethyl]carbamoyl]-4-hydroxy-pyrrolidine-1-carboxylate (50.00 mg, 127 umol, Intermediate UM) in DCM (1 mL) was added HCl/dioxane (4 M, 0.25 mL). The mixture was stirred at 25° C. for 1 hr. On completion, the reaction mixture was concentrated under reduced pressure to give the title compound (50 mg, HCl) as a white solid. LC-MS (ESI⁺) m/z293.1 (M+H)⁺.

### (2-Chloro-4-ethynyl-6-methoxyphenyl)methanamine (Intermediate UR)

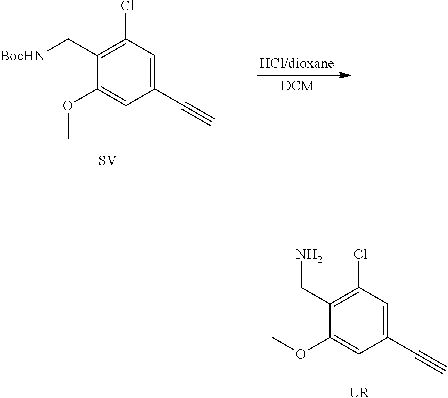

To a solution of tert-butyl N-[(2-chloro-4-ethynyl-6-methoxy-phenyl)methyl]carbamate (0.15 g, 507.2 umol, Intermediate SV) in DCM (5 mL) was added HCl/dioxane (4 M, 1 mL). The mixture was stirred at 20° C. for 3 hrs. On completion, the reaction was filtered to afford the title compound (0.11 g, 84% yield, HCl) as a white solid. LC-MS (ESI⁺) m/z 196.2 (M+H)⁺.

### (2S,4R)-1-((S)-2-amino-3,3-dimethylbutanoyl)-N-(2-chloro-4-ethynyl-6-methoxybenzyl)-4-hydroxypyrrolidine-2-carboxamide (Intermediate US)

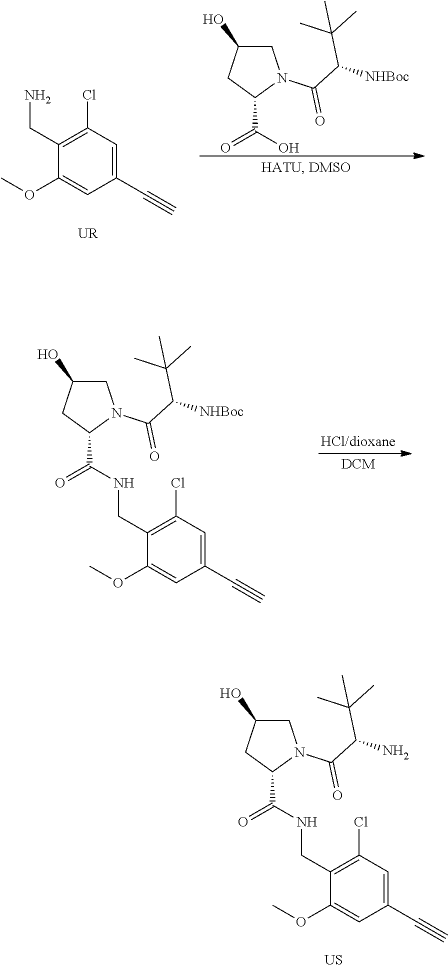

Step 1-Tert-butyl ((S)-1-((2S,4R)-2-((2-chloro-4-ethynyl-6-methoxybenzyl)carbamoyl)-4-hydroxypyrrolidin-1-yl)-3,3-dimethyl-1-oxobutan-2-yl)carbamate. To a solution of (2S,4R)-1-[(2S)-2-(tert-butoxycarbonylamino)-3,3-dimethyl-butanoyl]-4-hydroxy-pyrrolidine-2-carboxylic acid (100 mg, 290 umol, CAS #630421-46-4) in DMSO (5 mL) was added HATU (117 mg, 307 umol). After 10 min of stirring, DIEA (83.5 mg, 646.3 umol) and (2-chloro-4-ethynyl-6-methoxy-phenyl)methanamine (50 mg, 215.4 umol, HCl, Intermediate UR) were added and the mixture was stirred at 20° C. for 2 hrs. On completion, the reaction was filtered and the filtrate was the crude. The crude was purified by reversed phase HPLC (0.1% FA condition) to afford the title compound (0.1 g, 89% yield) as a yellow solid. LC-MS (ESI⁺) m/z 522.3 (M+H)⁺.

Step 2-(2S,4R)-1-((S)-2-amino-3,3-dimethylbutanoyl)-N-(2-chloro-4-ethynyl-6-methoxybenzyl)-4-hydroxypyrrolidine-2-carboxamide. To a solution of tert-butyl N-[(1S)-1-[(2S,4R)-2-[(2-chloro-4-ethynyl-6-methoxy-phenyl)methylcarbamoyl]-4-hydroxy-pyrrolidine-1-carbonyl]-2,2-dimethyl-propyl]carbamate (0.1 g, 191.6 umol) in DCM (1 mL) was added HCl/dioxane (4 M, 0.2 mL). The mixture was stirred at 20° C. for 3 hrs. On completion, the reaction was filtered and the filter cake was washed with DCM (2 mL×3) the dried to afford the title compound (80 mg, 82% yield, HCl) as a white solid. LC-MS (ESI⁺) m/z 422.2 (M+H)⁺.

### Ethyl 3-methyl-2-(3-(6-oxo-2-azaspiro[3.3]heptan-2-yl)isoxazol-5-yl)butanoate (Intermediate FH)

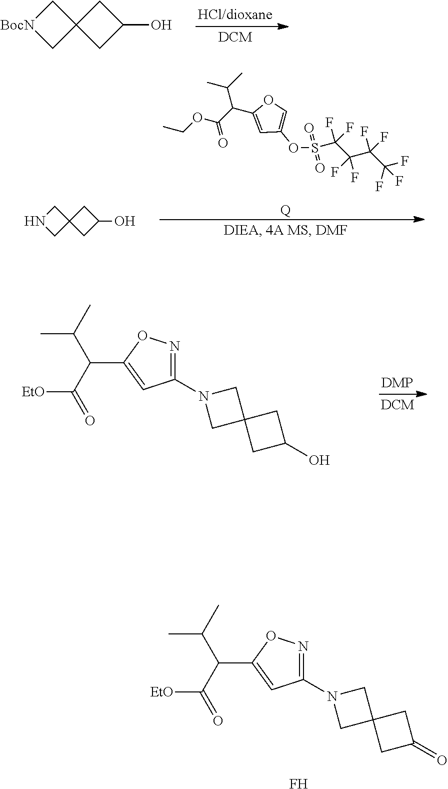

Step 1-2-Azaspiro[3.3]heptan-6-ol. To a solution of tert-butyl 6-hydroxy-2-azaspiro[3.3]heptane-2-carboxylate (995 mg, 4.67 mmol, CAS #1147557-97-8) in DCM (20 mL) was added HCl/dioxane (4 M, 10 mL). The mixture was stirred at 20° C. for 2 hours. On completion, the mixture was concentrated under reduced pressure to give the title compound (430 mg, HCl) as a light yellow gum. LC-MS (ESI⁺) m/z 114.1 (M+H)⁺.

Step 2-Ethyl 2-(3-(6-hydroxy-2-azaspiro[3.3]heptan-2-yl)isoxazol-5-yl)-3-methylbutanoate. To a solution of ethyl 3-methyl-2-(3-(((perfluorobutyl)sulfonyl)oxy)isoxazol-5-yl)butanoate (2.00 g, 4.04 mmol, Intermediate Q) in DMF (10 mL) was added DIEA (1.57 g, 12.1 mmol, 2.11 mL), 2-azaspiro[3.3]heptan-6-ol (906 mg, 6.06 mmol, HCl) and 4 Å molecular sieves (4 g). The mixture was stirred at 130° C. for 1 hour. On completion, the reaction mixture was filtered and quenched with water (40 mL) at 20° C., then extracted with EtOAc (20 mL×3). The combined organic layers were washed with brine (20 mL×3), dried over anhydrous sodium sulfate, filtered and concentrated under reduced pressure to give a residue, The residue was purified by column chromatography (SiO₂, Petroleum ether/Ethyl acetate=5/1 to 2/1) to give the title compound (1.8 g, 72% yield) as a yellow oil. LC-MS (ESI⁺) m/z 309.0 (M+H)⁺.

Step 3-Ethyl 3-methyl-2-(3-(6-oxo-2-azaspiro[3.3]heptan-2-yl)isoxazol-5-yl)butanoate. To a solution of DMP (2.81 g, 6.62 mmol, 2.05 mL) in DCM (40 mL) was added ethyl 2-[3-(6-hydroxy-2-azaspiro[3.3]heptan-2-yl)isoxazol-5-yl]-3-methyl-butanoate (1.7 g, 5.51 mmol) at 0° C. The mixture was then stirred at 20° C. for 3 hours. On completion, the reaction mixture was quenched with NaS₂O₃ aqueous solution (20 mL) and saturated NaHCO₃ aqueous solution (20 mL) at 20° C., and then extracted with DCM (30 mL×3). The combined organic layer was washed with saturated NaHCO₃ aqueous solution (50 mL) four times, dried over anhydrous sodium sulfate, filtered and concentrated under reduced pressure to give a residue. The residue was purified by column chromatography (SiO₂, Petroleum ether/Ethyl acetate=5/1 to 2/1) to give the title compound (1.3 g, 2.72 mmol, 49% yield) as a white solid. LC-MS (ESI⁺) m/z 307.2 (M+H)⁺.

### 2-(3-(6-(4-((S)-2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)-2-azaspiro[3.3]heptan-2-yl)isoxazol-5-yl)-3-methylbutanoic acid (Intermediate UU)

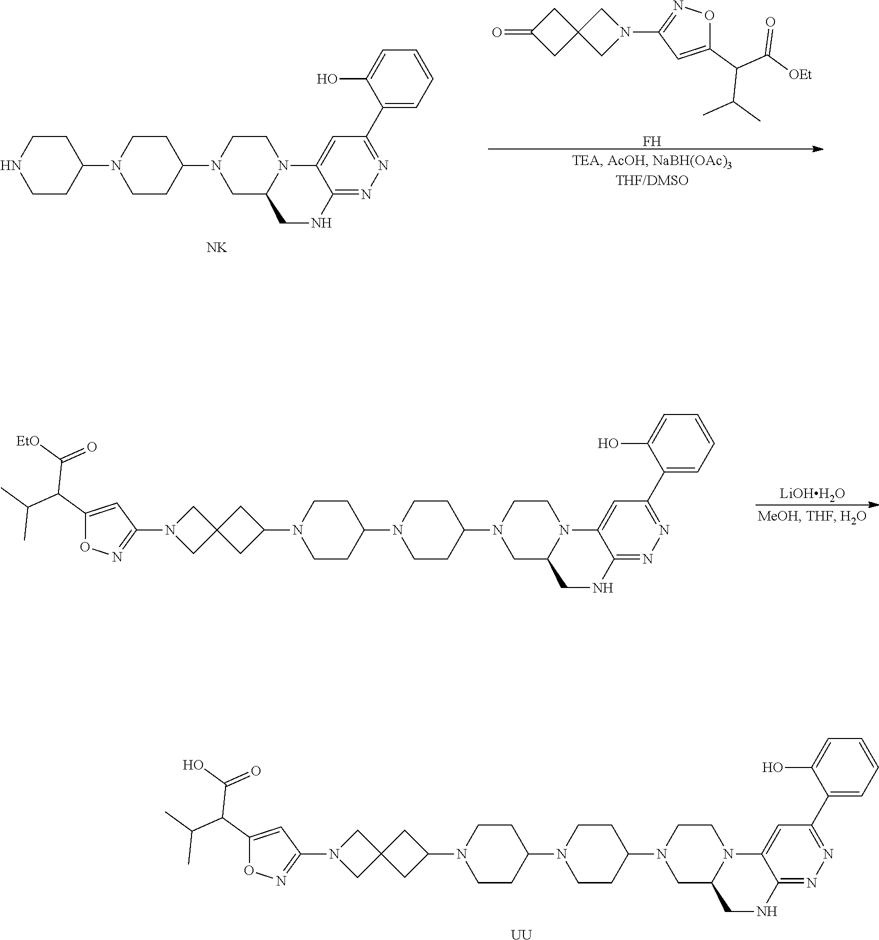

Step 1-Ethyl 2-(3-(6-(4-((S)-2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)-2-azaspiro[3.3]heptan-2-yl)isoxazol-5-yl)-3-methylbutanoate. To a solution of 2-[(1OS)-12-[1-(4-piperidyl)-4-piperidyl]-1,5,6,8,12-pentazatricyclo[8.4.0.0^{2,7}]tetradeca-2,4,6-trien-4-yl]phenol (528 mg, 1.18 mmol, Intermediate NK) in THF (5 mL) and DMSO (1 mL) was added TEA (247 mg, 2.45 mmol) and stirred for 0.5 hr. Then ethyl 3-methyl-2-[3-(6-oxo-2-azaspiro[3.3]heptan-2-yl)isoxazol-5-yl]butanoate (300 mg, 979 umol, Intermediate FH) and AcOH (147 mg, 2.45 mmol) was added and the mixture was stirred for 1 hr. Then NaBH(OAc)₃ (622 mg, 2.94 mmol) was added at 0° C., and the mixture was stirred at 25° C. for 1.5 hrs. On completion, the reaction mixture was quenched with MeOH (1 mL) and filtered and concentrated under reduced pressure to give a residue. The residue was purified by prep-HPLC (NH₃.H₂O condition) to give the title compound (280 mg, 34% yield) as a white solid. LC-MS (ESI⁺) m/z 740.4 (M+H)⁺.

Step 2-2-(3-(6-(4-((S)-2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)-2-azaspiro[3.3]heptan-2-yl)isoxazol-5-yl)-3-methylbutanoic acid. To a solution of ethyl 2-[3-[6-[4-[4-[(1OS)-4-(2-hydroxyphenyl)-1,5,6,8,12-pentazatricyclo[8.4.0.0^{2,7}]tetradeca-2,4,6-trien-12-yl]-1-piperidyl]-1-piperidyl]-2-azaspiro[3.3]heptan-2-yl]isoxazol-5-yl]-3-methyl-butanoate (280 mg, 336 umol) in THF (1 mL), H₂O (1 mL) and MeOH (1 mL) was added LiOH·H₂O (70.6 mg, 1.68 mmol). The mixture was stirred at 25° C. for 1 hr. On completion, the reaction mixture was adjusted the pH to 6 and filtered and concentrated under reduced pressure to give a residue. The residue was purified by prep-HPLC (FA condition) to give the title compound (80 mg, 31% yield, FA) as a white solid. LC-MS (ESI⁺) m/z 712.4 (M+H)⁺.

### (S)-2-(6-(5-(1-(2-azaspiro[3.3]heptan-6-yl)piperidin-4-yl)pyrimidin-2-yl)-5-(hydroxymethyl)-6,7,8,9-tetrahydro-5H-pyrido[3′,4′:4,5]pyrrolo[2,3-c]pyridazin-3-yl)phenol (Intermediate UV)

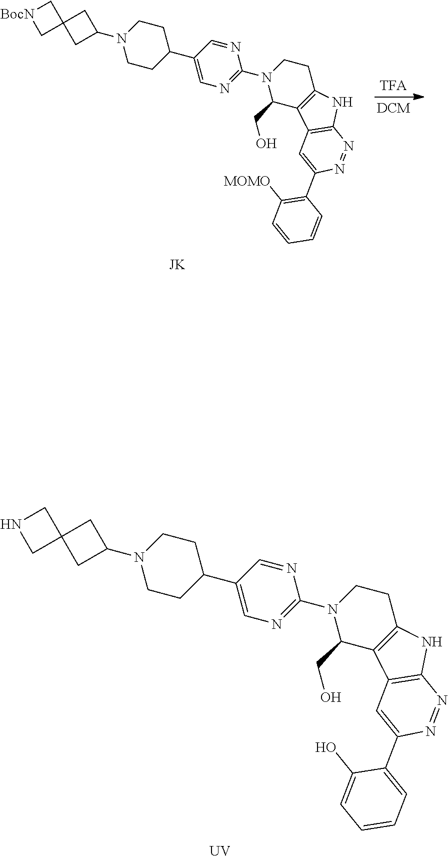

To a solution of tert-butyl 6-[4-[2-[(3S)-3-(hydroxymethyl)-12-[2-(methoxymethoxy)phenyl]-4,8,10,11-tetrazatricyclo[7.4.0.0^{2,7}]trideca-1(9),2(7),10,12-tetraen-4-yl]pyrimidin-5-yl]-1-piperidyl]-2-azaspiro[3.3]heptane-2-carboxylate (100 mg, 144 umol, Intermediate JK) in DCM (1 mL) was added TFA (462 mg, 4.05 mmol) and 4 Å molecular sieves (100 mg, 144 umol). The mixture was stirred at 25° C. for 30 mins. On completion, the residue was purified by reversed phase flash chromatography (FA condition) to give the title compound (50 mg, 38% yield, FA) as a yellow solid. LC-MS (ESI⁺) m/z 553.3 (M+H)⁺.

### (S)-2-(5-methyl-6-(5-(piperidin-4-yl)thiazol-2-yl)-6,7,8,9-tetrahydro-5H-pyrido[3′,4′:4,5]pyrrolo[2,3-c]pyridazin-3-yl)phenol (Intermediate UW)

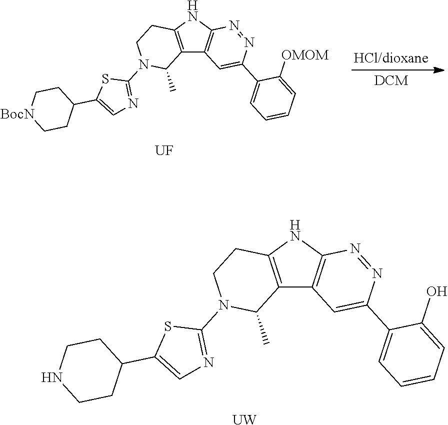

To a solution of tert-butyl 4-[2-[(3S)-12-[2-(methoxymethoxy)phenyl]-3-methyl-4,8,10,11-tetrazatricyclo[7.4.0.0^{2,7}]trideca-1(9),2(7),10,12-tetraen-4-yl]thiazol-5-yl]piperidine-1-carboxylate (110 mg, 186 umol, Intermediate UF) was added in DCM (2 mL) and HCl/dioxane (0.5 mL). The mixture was stirred at 25° C. for 1 hr. On completion, the reaction mixture was filtered and concentrated under reduced pressure to give the title compound (100 mg) as a yellow solid. LC-MS (ESI+) m/z 447.3 (M+H)⁺.

### (S)-2-(6-(5-(1-(2-azaspiro[3.3]heptan-6-yl)piperidin-4-yl)thiazol-2-yl)-5-methyl-6,7,8,9-tetrahydro-5H-pyrido[3′,4′:4,5]pyrrolo[2,3-c]pyridazin-3-yl)phenol (Intermediate UX)

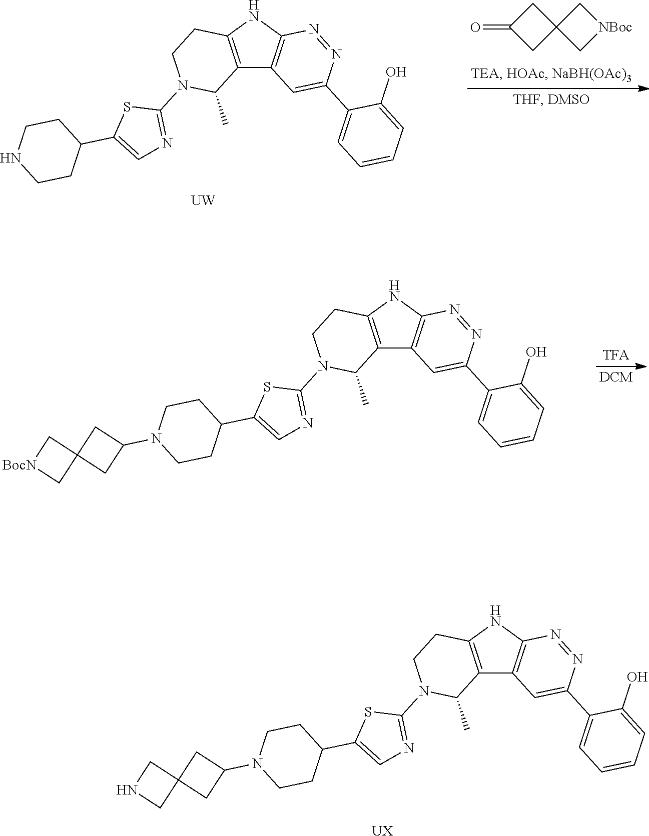

Step 1-(S)-tert-butyl 6-(4-(2-(3-(2-hydroxyphenyl)-5-methyl-7,8-dihydro-5H-pyrido[3′,4′:4,5]pyrrolo[2,3-c]pyridazin-6(9H)-yl)thiazol-5-yl)piperidin-1-yl)-2-azaspiro[3.3]heptane-2-carboxylate. To a solution of 2-[(3S)-3-methyl-4-[5-(4-piperidyl)thiazol-2-yl]-4,8,10,11-tetrazatricyclo[7.4.0.0¹′7]trideca-1(9),2(7),10,12-tetraen-12-yl]phenol (100 mg, 207 umol, Intermediate UW) in THF (2 mL) and DMSO (0.5 mL) was added TEA (41.9 mg, 414 umol) and stirred at 25° C. for 30 minutes. Then, tert-butyl 6-oxo-2-azaspiro[3.3]heptane-2-carboxylate (87.5 mg, 414 umol, CAS #1181816-12-5) and HOAc (31.1 mg, 518 umol) were added and stirred at 25° C. for 30 minutes. Finally, NaBH(OAc)₃ (110 mg, 518 umol) was added and the mixture was stirred at 0-25° C. for 3 hrs. On completion, the reaction mixture was filtered and concentrated under reduced pressure to give a residue. The crude product was purified by reversed-phase HPLC (0.1% FA condition) to give the title compound (110 mg, 77% yield) as a yellow solid. LC-MS (ESI+) m/z 642.6 (M+H)⁺.

Step 2-(S)-2-(6-(5-(1-(2-azaspiro[3.3]heptan-6-yl)piperidin-4-yl)thiazol-2-yl)-5-methyl-6,7,8,9-tetrahydro-5H-pyrido[3′,4′:4,5]pyrrolo[2,3-c]pyridazin-3-yl)phenol. To a solution of tert-butyl 6-[4-[2-[(3S)-12-(2-hydroxyphenyl)-3-methyl-4,8,10,11-tetrazatricyclo[7.4.0.0^{2,7}]trideca-1(9),2(7),10,12-tetraen-4-yl]thiazol-5-yl]-1-piperidyl]-2-azaspiro[3.3]heptane-2-carboxylate (110 mg, 171 umol) was added in TFA (0.3 mL) and DCM (1.5 mL). The mixture was then stirred at 25° C. for 1 hour. On completion, the reaction mixture was filtered and concentrated under reduced pressure to give the title compound (110 mg) as a yellow solid. LC-MS (ESI+) m/z 542.4 (M+H)⁺.

### Benzyl 2-(1,3-dioxoisoindolin-2-yl)-3,3-dimethyl-5-oxopentanoate (Intermediate UY) and 5-(benzyloxy)-4-(1,3-dioxoisoindolin-2-yl)-3,3-dimethyl-5-oxopentanoic acid (Intermediate VO)

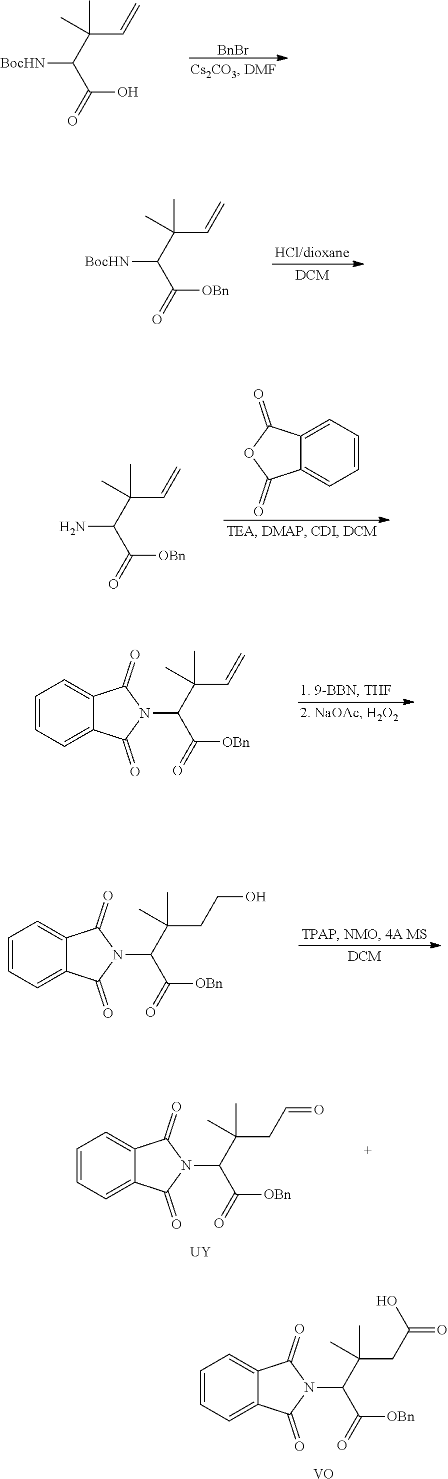

Step 1-Benzyl 2-((tert-butoxycarbonyl)amino)-3,3-dimethylpent-4-enoate. To a solution of 2-((tert-butoxycarbonyl)amino)-3,3-dimethylpent-4-enoic acid (30 g, 123 mmol) in DMF (400 mL) was added Cs₂CO₃ (40.1 g, 123 mmol) and bromomethylbenzene (23.2 g, 135 mmol) at 0° C., then the reaction was stirred at 25° C. for 12 hrs. On completion, the reaction mixture was quenched with water (1 L) and extracted by ethyl acetate (3×500 mL). The extracts were washed by brine (500 mL) and dried over anhydrous sodium sulfate, filtered and concentrated in vacuo to get the crude residue. The residue was purified by column chromatography (SiO₂, Petroleum ether/Ethyl acetate=20/1 to 15/1) to give the title compound (870 mg, 72% yield) as a yellow oil. LC-MS (ESI+) m/z 234.1 (M−99)⁺.

Step 2-Benzyl 2-amino-3,3-dimethylpent-4-enoate. To a solution of benzyl 2-((tert-butoxycarbonyl)amino)-3,3-dimethylpent-4-enoate (25 g, 74.9 mmol) in DCM (250 mL) was added HCl/dioxane (4 M, 60.00 mL) at 25° C., then the mixture was stirred at 25° C. for 2 hrs. On completion, the reaction mixture was filtered and concentrated in vacuo to give the title compound (49 g) as white solid. LC-MS (ESI⁺) m/z 234.0. (M+H)⁺.

Step 3-Benzyl 2-(1,3-dioxoisoindolin-2-yl)-3,3-dimethylpent-4-enoate. To a solution of isobenzofuran-1,3-dione (29.6 g, 200 mmol) and benzyl 2-amino-3,3-dimethylpent-4-enoate (49 g, 181 mmol) in DCM (160 mL) was added TEA (36.7 g, 363 mmol, 50.5 mL) and DMAP (2.22 g, 18.2 mmol) at 25° C., then the mixture was stirred at 25° C. for 12 hrs. Next, CDI (58.9 g, 363 mmol) was added at 25° C., and the mixture was stirred at 25° C. for 1 hr. On completion, the reaction mixture was filtered and concentrated in vacuo to get the crude residue. The residue was purified by column chromatography (SiO₂, Petroleum ether/Ethyl acetate=5/1) to give the title compound (64 g, 95% yield) as colorless oil. ¹H NMR (400 MHz, DMSO-d₆) δ=7.94-7.87 (m, 4H), 7.27-7.18 (m, 5H), 6.12 (dd, J=10.8, 17.5 Hz, 1H), 5.75 (s, 1H), 5.00 (d, J=0.8 Hz, 1H), 4.97-4.90 (m, 2H), 4.81 (s, 1H), 1.21 (s, 6H). LC-MS (ESI+) m/z 364.2. (M+H)⁺.

Step 4-Benzyl 2-(1,3-dioxoisoindolin-2-yl)-5-hydroxy-3,3-dimethylpentanoate. To a solution of benzyl 2-(1,3-dioxoisoindolin-2-yl)-3,3-dimethylpent-4-enoate (64 g, 176 mmol,) in THF (363 mL) was added 9-borabicyclo[3.3.1]nonane (0.5 M, 880 mL) dropwise at 0° C. After stirring at 25° C. for 1 hour, the mixture was recooled to 0° C. and H₂O₂(199 g, 1.76 mol, 169 mL, 30% solution) and sodium;acetate (4 M, 220 mL) at 0° C. was added slowly. Then the mixture was stirred at 25° C. for 12 hrs. On completion, the reaction mixture was quenched with saturated NaHSO₃ (800 mL) at 0° C., and then diluted with ethyl acetate (200 mL) and extracted with ethyl acetate (600 mL×3). The combined organic layers were washed with sat. NaCl (800 mL), dried over Na₂SO₄, filtered and concentrated under reduced pressure to give a residue. The residue was purified by column chromatography (SiO₂, Petroleum ether/Ethyl acetate=5/1 to 2/1) to give the title compound (41 g, 40% yield) as colorless oil.LC-MS (ESI⁺) m/z 382.3 (M+H)⁺.

Step 5-Benzyl 2-(1,3-dioxoisoindolin-2-yl)-3,3-dimethyl-5-oxopentanoate and 5-(benzyloxy)-4-(1,3-dioxoisoindolin-2-yl)-3,3-dimethyl-5-oxopentanoic acid. To a solution of benzyl 2-(1,3-dioxoisoindolin-2-yl)-5-hydroxy-3,3-dimethylpentanoate (10 g, 26.2 mmol) in DCM (100 mL) was added 4 Å molecular sieves (26.2 mmol), NMO (7.68 g, 65.5 mmol) and oxido(trioxo)ruthenium;tetrapropylammonium (921 mg, 2.62 mmol) at 25° C., then the mixture was stirred at 25° C. for 1 hr. On completion, the reaction mixture was filtered and concentrated in vacuo to get the crude residue. The residue was purified by column chromatography (SiO₂, Petroleum ether/Ethyl acetate=5/1) to give Benzyl 2-(1,3-dioxoisoindolin-2-yl)-3,3-dimethyl-5-oxopentanoate (9 g, 40% yield) as colorless oil (LC-MS (ESI⁺) m/z 380.3 (M+H)⁺) and 5-(benzyloxy)-4-(1,3-dioxoisoindolin-2-yl)-3,3-dimethyl-5-oxopentanoic acid (6 g, 40% yield) as a black oil (LC-MS (ESI+) m/z 396.2 (M+H)⁺).

### 3,3-dimethyl-5-morpholino-2-((phenoxycarbonyl)amino)pentanoic acid (Intermediate UZ)

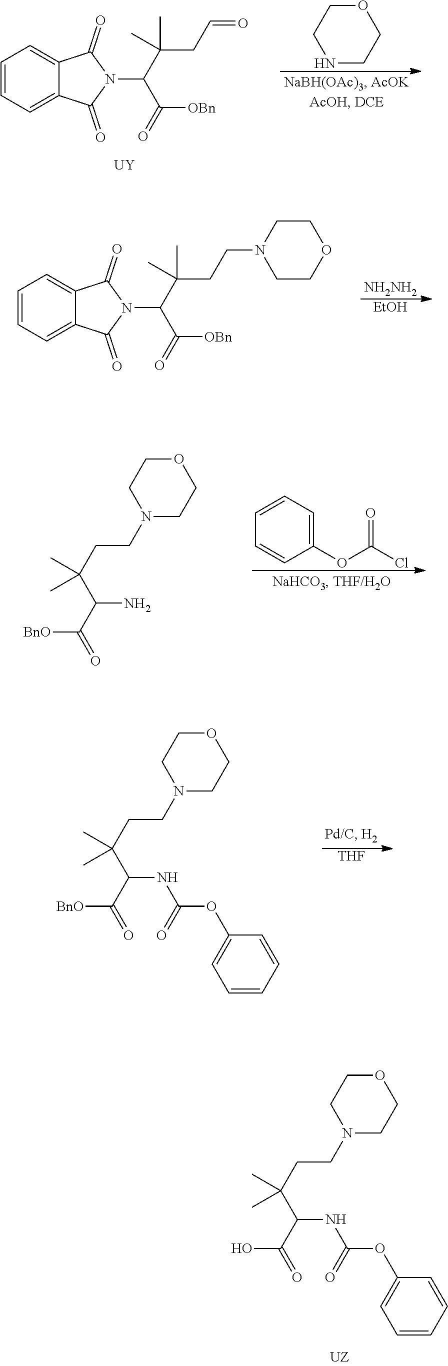

Step 1-Benzyl 2-(1,3-dioxoisoindolin-2-yl)-3,3-dimethyl-5-morpholinopentanoate. To a solution of benzyl 2-(1,3-dioxoisoindolin-2-yl)-3,3-dimethyl-5-oxopentanoate (2.5 g, 6.59 mmol, Intermediate UY) and morpholine (5.74 g, 65.9 mmol) in DCE (80 mL) was added AcOH (1.98 g, 33.0 mmol) at 25° C., then the reaction was stirred at 25° C. for 1 hr. Next, NaBH(OAc)₃ (2.79 g, 13.2 mmol) was added at 0° C., and the reaction was stirred at 25° C. for 1 hr. On completion, the reaction mixture was filtered and concentrated in vacuo to give the title compound (3 g, 89% yield) as colorless oil. LC-MS (ESI⁺) m/z 451.59. (M+H)⁺.

Step 2-Benzyl 2-amino-3,3-dimethyl-5-morpholinopentanoate. To a solution of benzyl benzyl 2-amino-3,3-dimethyl-5-morpholinopentanoate (1.40 g, 3.11 mmol) in EtOH (10 mL) was added NH₂NH₂·H₂O (1.24 g, 24.7 mmol, 85% solution) at 25° C., then the reaction was stirred at 60° C. for 12 hrs. On completion, the reaction mixture was diluted by water (10 mL) and extracted by ethyl acetate (3×10 mL). The extracts were washed with brine (10 mL) and dried over anhydrous sodium sulfate, filtered and concentrated in vacuo to give the title compound (280 mg, 85% yield) as colorless oil. LC-MS (ESI+) m/z 321.1. (M+H)⁺.

Step 3-Benzyl 3,3-dimethyl-5-morpholino-2-((phenoxycarbonyl)amino)pentanoate. To a solution of benzyl benzyl 2-amino-3,3-dimethyl-5-morpholinopentanoate (280 mg, 874 umol) in THF (3 mL) and H₂O (1.5 mL) was added NaHCO₃ (367 mg, 4.37 mmol) and phenyl carbonochloridate (205 mg, 1.31 mmol) at 0° C., then the reaction was stirred at 25° C. for 0.5 hr. On completion, the reaction mixture was filtered and concentrated in vacuo to get the crude residue. The residue was purified by column chromatography (SiO₂, DCM: MeOH=20:1/10:1) to give the title compound (260 mg, 67% yield) as colorless oil. LC-MS (ESI⁺) m/z 441.2. (M+H)⁺.

Step 4-3,3-Dimethyl-5-morpholino-2-((phenoxycarbonyl)amino)pentanoic acid. To a solution of benzyl 3,3-dimethyl-5-morpholino-2-((phenoxycarbonyl)amino)pentanoate (260 mg, 590 umol) in THF (5 mL) was added Pd/C (300 mg, 10 wt %) at 25° C. Then the reaction was stirred at 25° C. under H₂ (15 Psi) at 25° C. for 18 hrs. On completion, the reaction was filtered through celite carefully, then the filtrate was concentrated under reduced pressure to give the title compound (310 mg) as colorless oil. LC-MS (ESI⁺) m/z 351.3. (M+H)⁺.

### Phenyl (1-((2S,4R)-2-((4-ethynylbenzyl)carbamoyl)-4-hydroxypyrrolidin-1-yl)-3,3-dimethyl-5-morpholino-1-oxopentan-2-yl)carbamate (Intermediate VA)

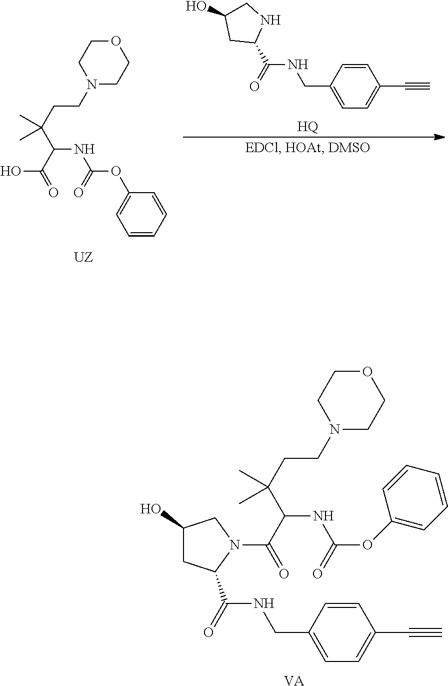

To a solution of 3,3-dimethyl-5-morpholino-2-((phenoxycarbonyl)amino)pentanoic acid (150 mg, 428 umol, Intermediate UZ) in DMSO (3 mL) was added EDCI (123 mg, 642 umol), HOAt (87.4 mg, 642 umol), and DIEA (277 mg, 2.14 mmol) at 25° C., then the mixture was stirred at 25° C. for 10 minutes. Then (2S,4R)—N-(4-ethynylbenzyl)-4-hydroxypyrrolidine-2-carboxamide (105 mg, 428 umol, Intermediate HQ) was added at 25° C., and the reaction was stirred at 25° C. for 1 hr. On completion, the reaction mixture was concentrated in vacuo to get the crude residue. The crude product was purified by reversed-phase HPLC (0.8 g/L NH₄HCO₃) to give the title compound (90 mg, 30% yield) as yellow solid. LC-MS (ESI+) m/z 577.2. (M+H)⁺.

### (R)-2-(((1-(benzyloxy)-5-(4-(tert-butoxycarbonyl)piperazin-1-yl)-3,3-dimethyl-1-oxopentan-2-yl)-12-azaneyl)carbonyl)benzoic acid (Intermediate VB) and (S)-2-((1-(benzyloxy)-5-(4-(tert-butoxycarbonyl)piperazin-1-yl)-3,3-dimethyl-1-oxopentan-2-yl)carbamoyl)benzoic acid (Intermediate VC)

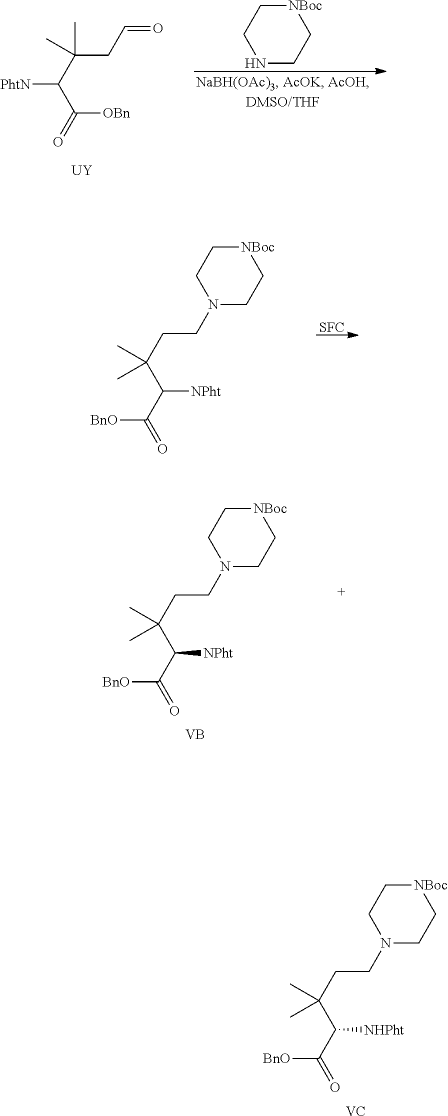

To a solution of benzyl 2-(1,3-dioxoisoindolin-2-yl)-3,3-dimethyl-5-oxo-pentanoate (2 g, 5.27 mmol, Intermediate UY), tert-butyl piperazine-1-carboxylate (1.18 g, 6.33 mmol) in THF (30 mL) was added HOAc (949 mg, 15.8 mmol) stirred at 0° C. for 0.5 hr, then NaBH(OAc)₃ (3.35 g, 15.8 mmol,) was added. The mixture was stirred at 0-25° C. for 1.5 hrs. On completion, the residue was diluted with H₂O (30 mL) and extracted with EA (30 mL×3). The combined organic layers were washed with aqueous NH4C₁ (30 mL×3), dried over Na₂SO₄, filtered and concentrated under reduced pressure to give a residue. The residue was purified by column chromatography (SiO₂, DCM: MeOH=10/0 to 10/1) to give an off-white oil. This oil was then further separated by SFC (column: DAICEL CHIRALCEL OX (250 mm*30 mm,10 um);mobile phase: [0.1% NH₃H2O IPA];B %: 15%-15%, A3.6; 86 min) to give the title compound (0.7 g, 99% yield) as a white solid (R)-2-(((1-(benzyloxy)-5-(4-(tert-butoxycarbonyl)piperazin-1-yl)-3,3-dimethyl-1-oxopentan-2-yl)-12-azaneyl)carbonyl)benzoic acid and (S)-2-((1-(benzyloxy)-5-(4-(tert-butoxycarbonyl)piperazin-1-yl)-3,3-dimethyl-1-oxopentan-2-yl)carbamoyl)benzoic acid (0.7 g, 97% yield) as a white solid. LC-MS (ESI⁺) m/z 550.5(M+H)⁺ for both isomers. Absolute stereochemistry assigned arbitrarily.

### (R)-5-(4-(tert-butoxycarbonyl)piperazin-1-yl)-3,3-dimethyl-2-((phenoxycarbonyl)amino)pentanoic acid (Intermediate VD)

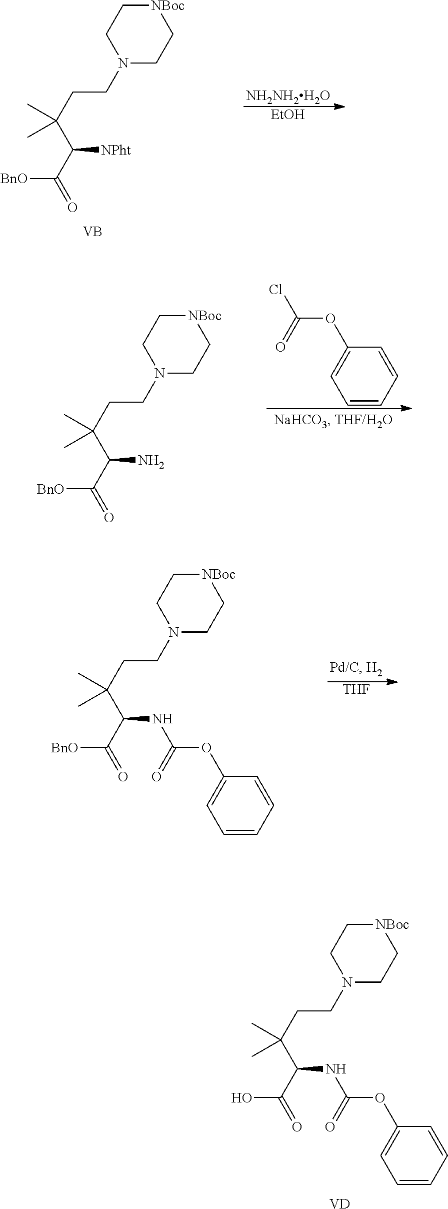

Step 1-(R)-tert-butyl 4-(4-amino-5-(benzyloxy)-3,3-dimethyl-5-oxopentyl)piperazine-1-carboxylate. To a solution of tert-butyl 4-[(4R)-5-benzyloxy-4-(1,3-dioxoisoindolin-2-yl)-3,3-dimethyl-5-oxo-pentyl]piperazine-1-carboxylate (600 mg, 1.09 mmol, Intermediate VB) in EtOH (10 mL) was added N₂H₄·H₂O (551 mg, 10.9 mmol, 85% solution). The mixture was stirred at 25° C. for 12 hrs. On completion, the mixture was diluted with H₂O (30 mL) and extracted with EA (30 mL×3). The combined organic layers were washed with aqueous NaCl (30 mL×3), dried over Na₂SO₄, filtered and concentrated under reduced pressure to give the title compound (450 mg) as a gray solid. LC-MS (ESI⁺) m/z 420.4(M+H)⁺.

Step 2-(R)-tert-butyl 4-(5-(benzyloxy)-3,3-dimethyl-5-oxo-4-((phenoxycarbonyl)amino)pentyl)piperazine-1-carboxylate. To a solution of tert-butyl 4-[(4R)-4-amino-5-benzyloxy-3,3-dimethyl-5-oxo-pentyl]piperazine-1-carboxylate (450 mg, 1.07 mmol) in THF (6 mL) and H₂O (2 mL) was added NaHCO₃ (360 mg, 4.29 mmol) and phenyl carbonochloridate (251 mg, 1.61 mmol). The mixture was stirred at 0-25° C. for 1 hr. On completion, the residue was diluted with H₂O (30 mL) and extracted with EA (30 mL×3). The combined organic layers were washed with aqueous NH₄Cl (30 mL×3), dried over Na₂SO₄, filtered and concentrated under reduced pressure to give a residue. The residue was purified by column chromatography (SiO₂, Petroleum ether/Ethyl acetate=10/0 to 3/1) to give the title compound (450 mg, 88% yield) as a white solid. LC-MS (ESI⁺) m/z 540.1(M+H)⁺.

Step 3-(R)-5-(4-(tert-butoxycarbonyl)piperazin-1-yl)-3,3-dimethyl-2-((phenoxycarbonyl)amino)pentanoic acid. A mixture of tert-butyl 4-[(4R)-5-benzyloxy-3,3-dimethyl-5-oxo-4-(phenoxycarbonylamino)pentyl]piperazine-1-carboxylate (450 mg, 833 umol) in THF (8 mL) was added Pd/C (300 mg, 8.34 mmol, 10 wt %). Then the mixture was purged with N₂ for 3 times, and then the mixture was stirred at 25° C. for 12 hrs under H₂(15 psi) atmosphere. The reaction mixture was filtered and concentrated under reduced pressure to give the title compound (450 mg) as a gray solid. LC-MS (ESI⁺) m/z 450.3 (M+H)⁺.

### Tert-butyl 4-((R)-5-((2S,4R)-2-((4-ethynylbenzyl)carbamoyl)-4-hydroxypyrrolidin-1-yl)-3,3-dimethyl-5-oxo-4-((phenoxycarbonyl)amino)pentyl)piperazine-1-carboxylate (Intermediate VE)

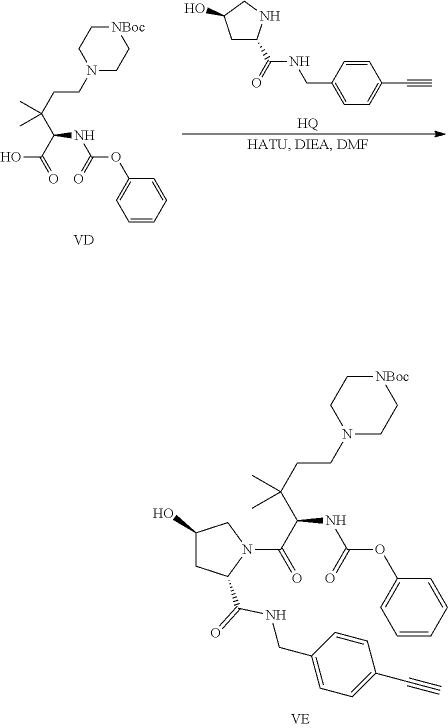

To a solution of (2R)-5-(4-tert-butoxycarbonylpiperazin-1-yl)-3,3-dimethyl-2-(phenoxycarbonylamino)pentanoic acid (400 mg, 889 umol, Intermediate VD) in DMSO (2 mL) and DMF (6 mL) was added DIEA (345 mg, 2.67 mmol), (2S,4R)—N-[(4-ethynylphenyl)methyl]-4-hydroxy-pyrrolidine-2-carboxamide (217 mg, 889 umol, Intermediate HQ) and HATU (372 mg, 978 umol). The mixture was then stirred at 0-25° C. for 1 hr. On completion, the residue was diluted with H₂O (30 mL) and extracted with EA (30 mL×3). The combined organic layers were washed with aqueous NaCl (30 mL×3), dried over Na₂SO₄, filtered and concentrated under reduced pressure to give the title compound (600 mg) as a yellowish-brown oil. LC-MS (ESI⁺) m/z 676.6 (M+H)⁺.

### (S)-5-(4-(tert-butoxycarbonyl)piperazin-1-yl)-3,3-dimethyl-2-((phenoxycarbonyl)amino)pentanoic acid (Intermediate VF)

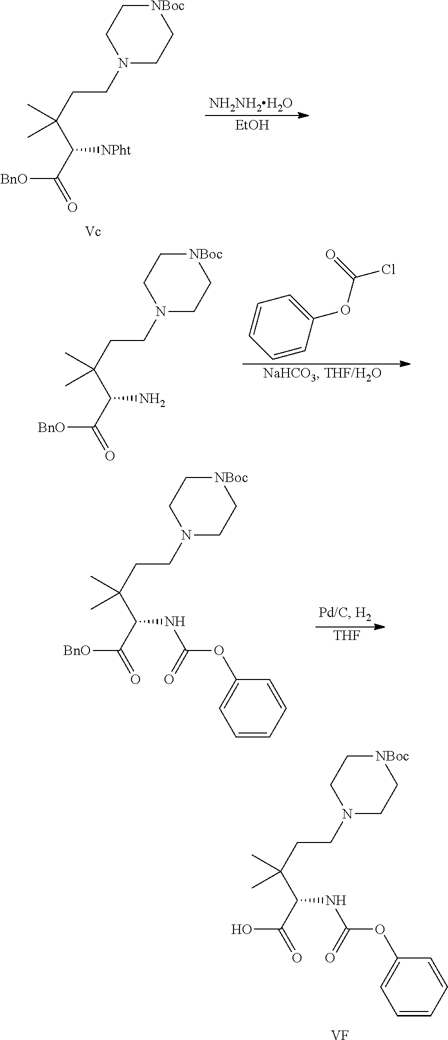

Step 1-(S)-tert-butyl 4-(4-amino-5-(benzyloxy)-3,3-dimethyl-5-oxopentyl)piperazine-1-carboxylate. To a solution of tert-butyl 4-[(4S)-5-benzyloxy-4-(1,3-dioxoisoindolin-2-yl)-3,3-dimethyl-5-oxo-pentyl]piperazine-1-carboxylate (650 mg, 1.18 mmol, Intermediate VC) in EtOH (10 mL) was added N₂H4.H₂O (598 mg, 11.9 mmol, 85% solution). The mixture was stirred at 25° C. for 12 hrs. On completion, the residue was diluted with H₂O (30 mL) and extracted with EA (30 mL×3). The combined organic layers were washed with aqueous NaCl (30 mL×3), dried over Na₂SO₄, filtered and concentrated under reduced pressure to give the title compound (460 mg) as a gray solid. LC-MS (ESI⁺) m/z 420.4(M+H)⁺.

Step 2-(S)-tert-butyl 4-(5-(benzyloxy)-3,3-dimethyl-5-oxo-4-((phenoxycarbonyl)amino)pentyl)piperazine-1-carboxylate. To a solution of tert-butyl 4-[(4S)-4-amino-5-benzyloxy-3,3-dimethyl-5-oxo-pentyl]piperazine-1-carboxylate (460 mg, 1.10 mmol) in THF (6 mL) and H₂O (2 mL) was added NaHCO₃ (368 mg, 4.39 mmol) and phenyl carbonochloridate (257 mg, 1.64 mmol). The mixture was stirred at 0-25° C. for 1 hr. The residue was diluted with H₂O (30 mL) and extracted with EA (30 mL×3). The combined organic layers were washed with aqueous NH4C₁ (30 mL×3), dried over Na₂SO₄, filtered and concentrated under reduced pressure to give a residue. The residue was purified by column chromatography (SiO₂, Petroleum ether/Ethyl acetate=10/0 to 3/1) to give the title compound (460 mg, 99% yield) as a white solid. LC-MS (ESI⁺) m/z 540.9(M+H)⁺.

Step 3-(S)-5-(4-(tert-butoxycarbonyl)piperazin-1-yl)-3,3-dimethyl-2-((phenoxycarbonyl)amino)pentanoic acid. A mixture of tert-butyl 4-[(4S)-5-benzyloxy-3,3-dimethyl-5-oxo-4-(phenoxycarbonylamino)pentyl]piperazine-1-carboxylate (450 mg, 833 umol) in THF (8 mL) was added Pd/C (300 mg, 8.34 mmol, 10 wt %) purged with N₂ three times, and then the mixture was stirred at 25° C. for 12 hrs under H₂ (15 psi) atmosphere. The reaction mixture was filtered and concentrated under reduced pressure to give the title compound (450 mg) as a gray solid. LC-MS (ESI⁺) m/z 450.4(M+H)⁺.

### Tert-butyl 4-((S)-5-((2S,4R)-2-((4-ethynylbenzyl)carbamoyl)-4-hydroxypyrrolidin-1-yl)-3,3-dimethyl-5-oxo-4-((phenoxycarbonyl)amino)pentyl)piperazine-1-carboxylate (Intermediate VG)

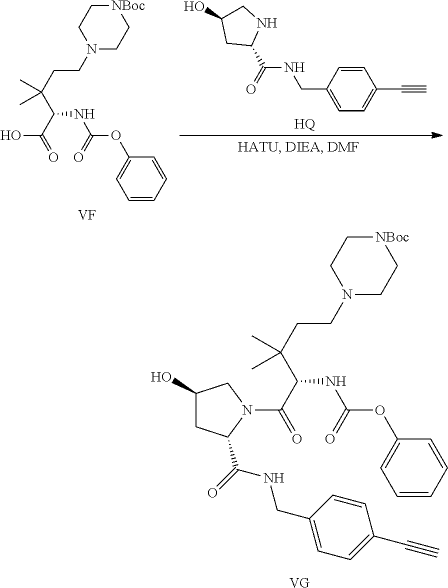

To a solution of (2S)-5-(4-tert-butoxycarbonylpiperazin-1-yl)-3,3-dimethyl-2-(phenoxycarbonylamino)pentanoic acid (400 mg, 889 umol, Intermediate VF) in DMSO (2 mL) and DMF (6 mL) was added DIEA (345 mg, 2.67 mmol), (2S,4R)—N-[(4-ethynylphenyl)methyl]-4-hydroxy-pyrrolidine-2-carboxamide (217 mg, 889 umol, Intermediate HQ) and HATU (372 mg, 978 umol). The mixture was stirred at 0-25° C. for 1 hr. On completion, the residue was diluted with H₂O (30 mL) and extracted with EA (30 mL×3). The combined organic layers were washed with aqueous NaCl (30 mL×3), dried over Na₂SO₄, filtered and concentrated under reduced pressure to give the title compound (650 mg) as a yellowish-brown oil. LC-MS (ESI⁺) m/z 676.6(M+H)⁺.

### (R)-1-tert-butyl 2-methyl 4-((S)-5-(benzyloxy)-4-(1,3-dioxoisoindolin-2-yl)-3,3-dimethyl-5-oxopentyl)piperazine-1,2-dicarboxylate (Intermediate VH) and (R)-1-tert-butyl 2-methyl 4-((R)-5-(benzyloxy)-4-(1,3-dioxoisoindolin-2-yl)-3,3-dimethyl-5-oxopentyl)piperazine-1,2-dicarboxylate (Intermediate VI)

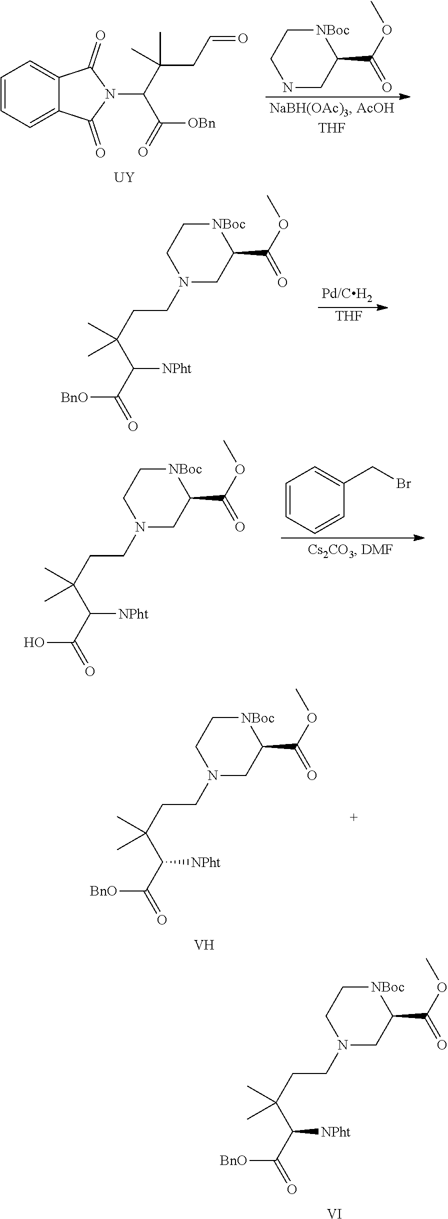

Step 1-(2R)-1-tert-butyl 2-methyl 4-(5-(benzyloxy)-4-(1,3-dioxoisoindolin-2-yl)-3,3-dimethyl-5-oxopentyl)piperazine-1,2-dicarboxylate. To a solution of 01-tert-butyl 02-methyl (2R)-piperazine-1,2-dicarboxylate (2.30 g, 9.39 mmol, CAS #252990-05-9) and benzyl 2-(1,3-dioxoisoindolin-2-yl)-3,3-dimethyl-5-oxopentanoate (1.7 g, 4.48 mmol, Intermediate UY) in THF (10 mL) was added AcOH (1.08 g, 17.9 mmol) at 25° C., then the mixture was stirred at 25° C. for 0.5 h. Next, NaBH(OAc)₃ (4.75 g, 22.4 mmol) was added at 0° C., and the mixture was stirred at 25° C. for 2 hrs. On completion, the reaction mixture was quenched/diluted by water (15 mL) and extracted with ethyl acetate (15 mL×3). The extracts were washed with brine (30 mL) and dried over anhydrous sodium sulfate, filtered and concentrated in vacuo to get the crude residue. The residue was purified by column chromatography (SiO₂, Petroleum ether/Ethyl acetate=5/1 to 4/1) to give the title compound (2.5 g, 87% yield) as colorless oil. LC-MS (ESI⁺) m/z 608.0 (M+H)⁺.

Step 2-5-((R)-4-(tert-butoxycarbonyl)-3-(methoxycarbonyl)piperazin-1-yl)-2-(1,3-dioxoisoindolin-2-yl)-3,3-dimethylpentanoic acid. To a solution of (2R)-1-tert-butyl 2-methyl 4-(5-(benzyloxy)-4-(1,3-dioxoisoindolin-2-yl)-3,3-dimethyl-5-oxopentyl)piperazine-1,2-dicarboxylate (2.5 g, 4.11 mmol) in THF (25 mL) was added Pd/C (2.5 g, 4.11 mmol, 10% purity) at 25° C., the mixture was stirred at 25° C. for 12 hours under H₂ atmosphere (15 Psi). On completion, the reaction mixture was filtered carefully and the catalyst was recycled, then concentrated in vacuo to get the crude residue. On completion, the reaction mixture was filtered and concentrated in vacuo to get the crude residue. Give the title compound (2 g, crude) as colorless solid. LC-MS (ESI⁺) m/z 518.9 (M+H)⁺.

Step 3-(R)-1-tert-butyl 2-methyl 4-((S)-5-(benzyloxy)-4-(1,3-dioxoisoindolin-2-yl)-3,3-dimethyl-5-oxopentyl)piperazine-1,2-dicarboxylate and (R)-1-tert-butyl 2-methyl 4-((R)-5-(benzyloxy)-4-(1,3-dioxoisoindolin-2-yl)-3,3-dimethyl-5-oxopentyl)piperazine-1,2-dicarboxylate. To a solution of 5—((R)-4-(tert-butoxycarbonyl)-3-(methoxycarbonyl)piperazin-1-yl)-2-(1,3-dioxoisoindolin-2-yl)-3,3-dimethylpentanoic acid (1.8 g, 3.48 mmol) in DMF (15 mL) was added (bromomethyl)benzene (654 mg, 3.83 mmol) and Cs₂CO₃ (1.13 g, 3.48 mmol) at 0° C., then the mixture was stirred at 25° C. for 12 hrs. On completion, the reaction mixture was quenched with water (20 mL) and extracted by ethyl acetate (3×15 mL). The extracts were washed with brine (30 mL) and dried over anhydrous sodium sulfate, filtered and concentrated in vacuo to get the crude residue. The residue was purified by column chromatography (SiO₂, Petroleum ether/Ethyl acetate=5/1) to give (R)-1-tert-butyl 2-methyl 4-((S)-5-(benzyloxy)-4-(1,3-dioxoisoindolin-2-yl)-3,3-dimethyl-5-oxopentyl)piperazine-1,2-dicarboxylate (900 mg, 37% yield) as colorless solid (LC-MS (ESI⁺) m/z 608.8 (M+H)⁺) and (R)-1-tert-butyl 2-methyl 4-((R)-5-(benzyloxy)-4-(1,3-dioxoisoindolin-2-yl)-3,3-dimethyl-5-oxopentyl)piperazine-1,2-dicarboxylate (640 mg, 32% yield) as colorless solid. LC-MS (ESI⁺) m/z 608.8 (M+H)⁺. The absolute stereochemistry was assigned arbitrarily.

### (S)-5-((R)-4-(tert-butoxycarbonyl)-3-(methoxycarbonyl)piperazin-1-yl)-3,3-dimethyl-2-((phenoxycarbonyl)amino)pentanoic acid (Intermediate VJ)

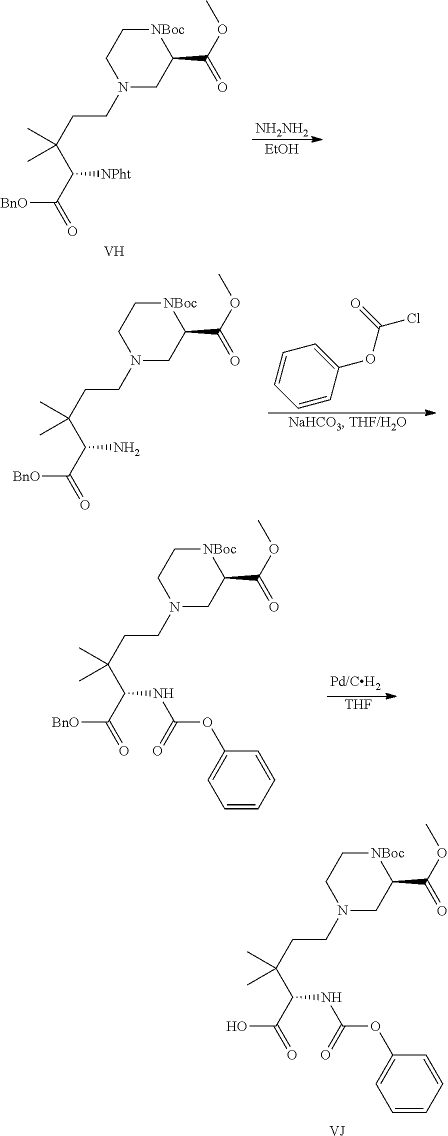

Step 1-(R)-1-tert-butyl 2-methyl 4-((S)-4-amino-5-(benzyloxy)-3,3-dimethyl-5-oxopentyl)piperazine-1,2-dicarboxylate. To a solution of 01-tert-butyl 02-methyl (2R)-4-[(4S)-5-benzyloxy-4-(1,3-dioxoisoindolin-2-yl)-3,3-dimethyl-5-oxo-pentyl]piperazine-1,2-dicarboxylate (600 mg, 987 umol, Intermediate VH) in EtOH (10 mL) was added NH₂NH₂.H₂O (494 mg, 9.87 mmol, 85% solution) at 25° C., then the mixture was then stirred at 25° C. for 12 hrs. On completion, the reaction mixture was concentrated in vacuo to get the crude residue. The residue was then diluted with water (30 mL) and extracted with ethyl acetate (3×20 mL). The extracts were washed with brine (20 mL) and dried over anhydrous sodium sulfate, filtered and concentrated in vacuo to give the title compound (700 g) as colorless oil. LC-MS (ESI⁺) m/z 478.3 (M+H)⁺.

Step 2-(R)-1-tert-butyl 2-methyl 4-((S)-5-(benzyloxy)-3,3-dimethyl-5-oxo-4-((phenoxycarbonyl)amino)pentyl)piperazine-1,2-dicarboxylate. To a solution of (R)-1-tert-butyl 2-methyl 4-((S)-4-amino-5-(benzyloxy)-3,3-dimethyl-5-oxopentyl)piperazine-1,2-dicarboxylate (700 mg, 1.47 mmol) in THF (8 mL) and H₂O (4 mL) was added NaHCO₃ (615 mg, 7.33 mmol) and phenyl carbonochloridate (344 mg, 2.20 mmol) at 0° C., then the reaction was stirred at 25° C. for 1 hr. On completion, the reaction mixture was diluted with water (20 mL) and extracted with ethyl acetate/dichloromethane (3×20 mL). The extracts were then washed with brine (20 mL) and dried over anhydrous sodium sulfate, filtered and concentrated in vacuo to give the crude residue. The residue was purified by column chromatography (SiO₂, Petroleum ether/Ethyl acetate=5/1 to 3/1) to give the title compound (550 mg) as obtained as a white solid. LC-MS (ESI⁺) m/z 598.1 (M+H)⁺.

Step 3-(S)-5-((R)-4-(tert-butoxycarbonyl)-3-(methoxycarbonyl)piperazin-1-yl)-3,3-dimethyl-2-((phenoxycarbonyl)amino)pentanoic acid. To a solution of (R)-1-tert-butyl 2-methyl 4-((S)-5-(benzyloxy)-3,3-dimethyl-5-oxo-4-((phenoxycarbonyl)amino)pentyl)piperazine-1,2-dicarboxylate (450 mg, 752 umol) in THF (5 mL) was added Pd/C (450 mg, 752 umol, 10 wt % p) at 25° C. The mixture was then stirred at 25° C. for 12 hrs under H₂ atmosphere (15 Psi). On completion, the reaction mixture was filtered carefully then concentrated in vacuo to give the title compound (425 mg) as colorless solid. LC-MS (ESI⁺) m/z 508.8 (M+H)⁺.

### (R)-1-tert-butyl 2-methyl 4-((S)-5-((2S,4R)-2-((4-ethynylbenzyl)carbamoyl)-4-hydroxypyrrolidin-1-yl)-3,3-dimethyl-5-oxo-4-((phenoxycarbonyl)amino)pentyl)piperazine-1,2-dicarboxylate (Intermediate VK)

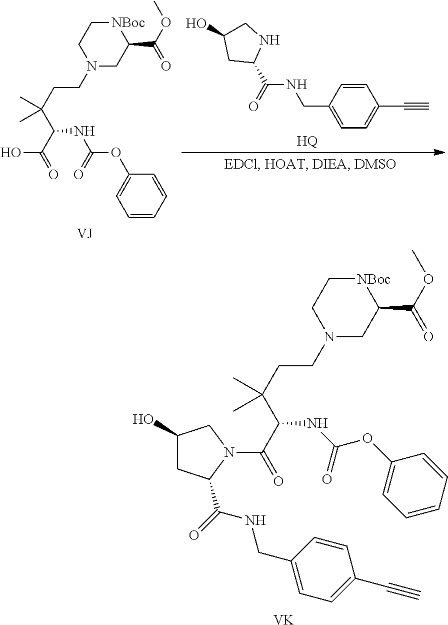

To a solution of (2S,4R)—N-(4-ethynylbenzyl)-4-hydroxypyrrolidine-2-carboxamide (204 mg, 837 umol, Intermediate HQ) and (S)-5-((R)-4-(tert-butoxycarbonyl)-3-(methoxycarbonyl)piperazin-1-yl)-3,3-dimethyl-2-((phenoxycarbonyl)amino)pentanoic acid (425 mg, 837 umol, Intermediate VJ) in DMSO (6 mL) was added HOAt (170 mg, 1.26 mmol), EDCI (240 mg, 1.26 mmol) and DIEA (432 mg, 3.35 mmol) at 25° C., then the mixture was stirred at 25° C. for 2 hrs. On completion, the reaction mixture was quenched with water (10 mL) and extracted by ethyl acetate (3×15 mL). The extracts were washed by brine (20 mL) and dried over anhydrous sodium sulfate, filtered and concentrated in vacuo to give the title compound (700 mg) as colorless oil. LC-MS (ESI⁺) m/z 734.6 (M+H)⁺.

### (R)-5-((R)-4-(tert-butoxycarbonyl)-3-(methoxycarbonyl)piperazin-1-yl)-3,3-dimethyl-2-((phenoxycarbonyl)amino)pentanoic acid (Intermediate VL)

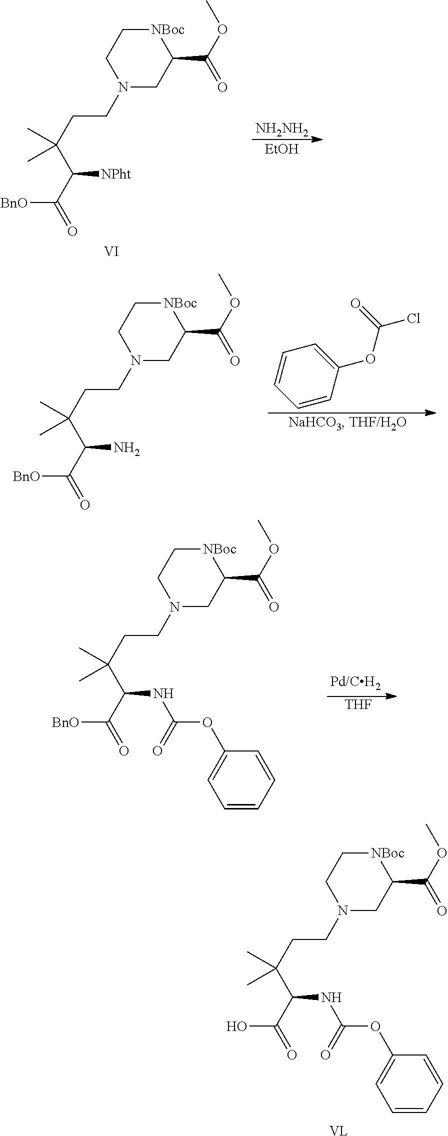

Step 1-(R)-1-tert-butyl 2-methyl 4-((R)-4-amino-5-(benzyloxy)-3,3-dimethyl-5-oxopentyl)piperazine-1,2-dicarboxylate. To a solution of 01-tert-butyl 02-methyl (2R)-4-[(4S)-5-benzyloxy-4-(1,3-dioxoisoindolin-2-yl)-3,3-dimethyl-5-oxopentyl]piperazine-1,2-dicarboxylate (850 mg, 1.40 mmol, Intermediate VI) in EtOH (10 mL) was added NH₂NH₂.H₂O (560 mg, 11.1 mmol) at 25° C., then the mixture was stirred at 25° C. for 12 hrs. On completion, the reaction mixture was concentrated in vacuo to get the crude residue. The residue was diluted with water (30 mL) and extracted by ethyl acetate (20 mL×3). The extracts were washed by brine (20 mL) and dried over anhydrous sodium sulfate, filtered and concentrated in vacuo to give the title compound (1 g) as colorless oil. LC-MS (ESI⁺) m/z 478.2 (M+H)⁺.

Step 2-(R)-1-tert-butyl 2-methyl 4-((R)-5-(benzyloxy)-3,3-dimethyl-5-oxo-4-((phenoxycarbonyl)amino)pentyl)piperazine-1,2-dicarboxylate. To a solution of (R)-1-tert-butyl 2-methyl 4-((R)-4-amino-5-(benzyloxy)-3,3-dimethyl-5-oxopentyl)piperazine-1,2-dicarboxylate (940 mg, 1.97 mmol) in THF (9 mL) and H₂O (4.5 mL) was added NaHCO₃ (826 mg, 9.84 mmol) and phenyl carbonochloridate (462 mg, 2.95 mmol) at 0° C., then the reaction was stirred at 25° C. for 1 hr. On completion, the reaction mixture was diluted with water (20 mL) and extracted with ethyl acetate/dichloromethane (20 mL×3). The extracts were washed with brine (20 mL) and dried over anhydrous sodium sulfate, filtered and concentrated in vacuo to get the crude residue. The residue was purified by column chromatography (SiO₂, Petroleum ether/Ethyl acetate=5/1 to 2/1) to give the title compound (570 mg) as a white solid. LC-MS (ESI⁺) m/z 598.1 (M+H)⁺.

Step 3-(R)-5-((R)-4-(tert-butoxycarbonyl)-3-(methoxycarbonyl)piperazin-1-yl)-3,3-dimethyl-2-((phenoxycarbonyl)amino)pentanoic acid. To a solution of (R)-1-tert-butyl 2-methyl 4-((R)-5-(benzyloxy)-3,3-dimethyl-5-oxo-4-((phenoxycarbonyl)amino)pentyl)piperazine-1,2-dicarboxylate (570 mg, 954 umol) in THF (10 mL) was added Pd/C (570 mg, 752 umol, 10 wt %) at 25° C., then the mixture was stirred at 25° C. for 12 hrs under H₂ atmosphere (15 Psi). On completion, the reaction mixture was filtered carefully, then the filtrate was concentrated in vacuo to give the title compound (530 mg, crude) as colorless solid. LC-MS (ESI⁺) m/z 508.2 (M+H)⁺.

### (R)-1-tert-butyl 2-methyl 4-((R)-5-((2S,4R)-2-((4-ethynylbenzyl)carbamoyl)-4-hydroxypyrrolidin-1-yl)-3,3-dimethyl-5-oxo-4-((phenoxycarbonyl)amino)pentyl)piperazine-1,2-dicarboxylate (Intermediate VM)

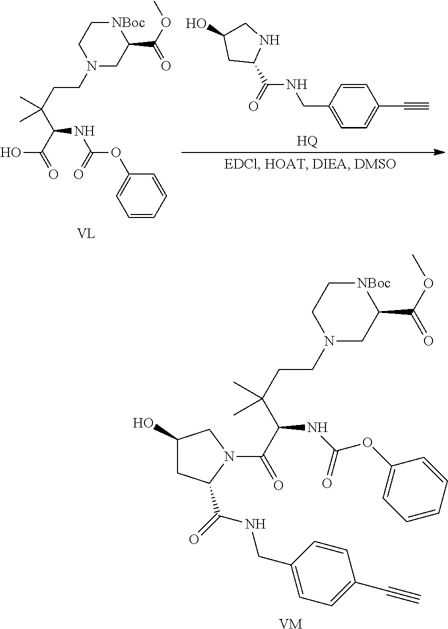

To a solution of (2S,4R)—N-(4-ethynylbenzyl)-4-hydroxypyrrolidine-2-carboxamide (204 mg, 837 umol, Intermediate HQ) and (R)-5-((R)-4-(tert-butoxycarbonyl)-3-(methoxycarbonyl)piperazin-1-yl)-3,3-dimethyl-2-((phenoxycarbonyl)amino)pentanoic acid (480 mg, 946 umol, Intermediate VL) in DMSO (6 mL) was added HOAt (193 mg, 1.42 mmol), EDCI (272 mg, 1.42 mmol) and DIEA (367 mg, 2.84 mmol) at 25° C., then the mixture was stirred at 25° C. for 2 hrs. On completion, the reaction mixture was quenched with water (10 mL) and extracted with ethyl acetate (15 mL×3). The extracts were washed with brine (20 mL) and dried over anhydrous sodium sulfate, filtered and concentrated in vacuo to give the title compound (736 mg) as colorless oil. LC-MS (ESI⁺) m/z 734.5 (M+H)⁺.

### Ethyl 3-methyl-2-(3-(3-oxopropyl)isoxazol-5-yl)butanoate (Intermediate VT)

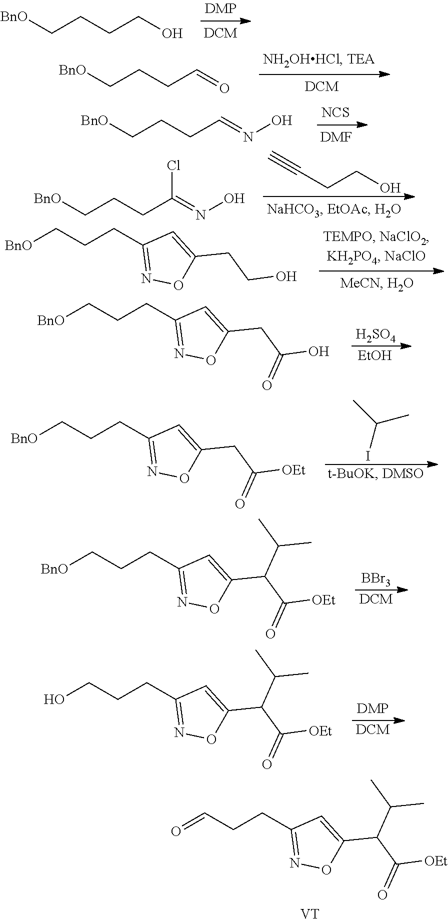

Step 1-4-(Benzyloxy)butanal. To a mixture of 4-(benzyloxy)butan-1-ol (5 g, 27.7 mmol, CAS #4541-14-4) in DCM (100 mL) was added DMP (17.7 g, 41.6 mmol). The mixture was stirred at 25° C. for 3 hrs. On completion, the reaction mixture was diluted with Na₂S₂O₃ (100 mL) and NaHCO₃ (100 mL), then extracted with DCM 300 mL (100 mL×3). The combined organic layers were dried over Na₂SO₄, filtered and concentrated under reduced pressure to give a residue. The residue was purified by column chromatography (SiO₂, Petroleum ether/Ethyl acetate=1/0 to 50/1) to give the title compound (3.78 g, 66% yield) as a white oil. ¹H NMR (400 MHz, DMSO-d6) δ=9.68 (t, J=1.6 Hz, 1H), 7.37-7.29 (m, 5H), 4.45 (s, 2H), 3.44 (t, J=6.4 Hz, 2H), 2.51-2.46 (m, 2H), 1.86-1.81 (m, 2H).

Step 2-(E)-4-(benzyloxy)butanal oxime. To a mixture of 4-(benzyloxy)butanal (30.8 g, 188 mmol) in DCM (350 mL) was added NH₂OH—HCl (15.6 g, 225 mmol) and TEA (56.9 g, 563 mmol). The mixture was stirred at 25° C. for 1 hr. On completion, the reaction mixture was diluted with H₂O (500 mL) and extracted with DCM (300 mL×3). The combined organic layers were dried over Na₂SO₄, filtered and concentrated under reduced pressure to give a residue. The residue was purified by column chromatography (SiO₂, Petroleum ether/Ethyl acetate=10/1 to 5/1) to give the title compound (35.4 g, 91% yield) as a yellow oil. LC-MS (ESI⁺) m/z 194.1 (M+H)⁺.

Step 3-(Z)-4-(benzyloxy)-N-hydroxybutanimidoyl chloride. To a mixture of (E)-4-(benzyloxy)butanal oxime (35 g, 181 mmol) in DMF (300 mL) was added NCS (29.0 g, 217 mmol). The mixture was stirred at 25° C. for 2 hrs. On completion, the reaction mixture was diluted with H₂O (300 mL) and extracted with EA (300 mL×3). The combined organic layers were dried over Na₂SO₄, filtered and concentrated under reduced pressure to give the title compound (40.6 g) as a yellow oil. LC-MS (ESI⁺) m/z 192.0 (M−34)⁺.

Step 4-2-(3-(3-(Benzyloxy)propyl)isoxazol-5-yl)ethanol. To a mixture of (Z)-4-(benzyloxy)-N-hydroxybutanimidoyl chloride (5 g, 22.0 mmol) in EtOAc (25 mL) and H₂O (25 mL) was added NaHCO₃ (2.31 g, 27.4 mmol) and but-3-yn-1-ol (1.54 g, 22.0 mmol). The mixture was stirred at 25° C. for 12 hrs. On completion, the reaction mixture was diluted with H₂O (200 mL) and extracted with EtOAc (200 mL×3). The combined organic layers were wash with brine (50 mL×2) and dried overNa₂SO₄, filtered and concentrated under reduced pressure to give a residue. The residue was purified by column chromatography (SiO₂, Petroleum ether/Ethyl acetate=10/1 to 5/1) to give the title compound (2.1 g, 33% yield) as a white oil. LC-MS (ESI⁺) m/z 262.2 (M+H)⁺.

Step 5-2-(3-(3-(Benzyloxy)propyl)isoxazol-5-yl)acetic acid. To a mixture of 2-(3-(3-(benzyloxy)propyl)isoxazol-5-yl)ethanol (6 g, 23.0 mmol) in MeCN (35 mL) was added TEMPO (361 mg, 2.30 mmol), and KH₂PO₄ (3.12 g, 23.0 mmol), then NaClO₂ (5.19 g, 57.4 mmol) in H₂O (15 mL) was added followed by NaClO (171 mg, 2.30 mmol). The mixture was then stirred at 60° C. for 12 hrs. On completion, the reaction mixture was diluted with H₂O (100 mL) and extracted with EtOAc (100 mL×3). The combined organic layers were wash with brine (50 mL×2) and dried over Na₂SO₄, filtered and concentrated under reduced pressure to give a residue. The residue was purified by column chromatography (SiO₂, Petroleum ether/Ethyl acetate=5/1 to 1/1) to give the title compound (3.1 g, 47% yield) as a white oil. LC-MS (ESI⁺) m/z 276.1 (M+H)⁺.

Step 6-Ethyl 2-(3-(3-(benzyloxy)propyl)isoxazol-5-yl)acetate. To a mixture of 2-(3-(3-(benzyloxy)propyl)isoxazol-5-yl)acetic acid (3.1 g, 11 mmol) in EtOH (31 mL) was added H₂SO₄ (71.3 mg, 727 umol). The mixture was stirred at 70° C. for 1.5 hrs. On completion, the reaction mixture was diluted with H₂O (100 mL) and extracted with EtOAc (100 mL×3). The combined organic layers were wash with brine (50 mL×2) and dried over Na₂SO₄, filtered and concentrated under reduced pressure to give a residue. The residue was purified by column chromatography (SiO₂, Petroleum ether/Ethyl acetate=10/1 to 6/1) to give the title compound (3.1 g, 90% yield) as a white oil. LC-MS (ESI⁺) m/z 304.4 (M+H)⁺.

Step 7-Ethyl 2-(3-(3-(benzyloxy)propyl)isoxazol-5-yl)-3-methylbutanoate. To a mixture of ethyl 2-(3-(3-(benzyloxy)propyl)isoxazol-5-yl)acetate (2.1 g, 6.92 mmol) in DMSO (20 mL) was added t-BuOK (932 mg, 8.31 mmol). The mixture was stirred at 25° C. for 30 mins. Then 2-iodopropane (1.29 g, 7.61 mmol) was added and he mixture was stirred at 25° C. for 2 hrs. On completion, the reaction mixture was diluted with NH₄Cl (100 mL) and extracted with DCM (100 mL×3). The combined organic layers were wash with brine (50 mL×2) and dried over Na₂SO₄, filtered and concentrated under reduced pressure to give a residue. The residue was purified by column chromatography (SiO₂, Petroleum ether/Ethyl acetate=1/0 to 20/1) to give the title compound (1.7 g, 64% yield) as a white oil. LC-MS (ESI⁺) m/z 346.4 (M+H)⁺.

Step 8-Ethyl 2-(3-(3-hydroxypropyl)isoxazol-5-yl)-3-methylbutanoate. To a mixture of ethyl 2-(3-(3-(benzyloxy)propyl)isoxazol-5-yl)-3-methylbutanoate (1.5 g, 4.34 mmol) in DCM (15 mL) was added BBr₃ (1.85 g, 7.38 mmol). The mixture was stirred at −78° C. for 2 hrs. On completion, the reaction mixture was diluted with H₂O (100 mL) and extracted with DCM (100 mL×3). The combined organic layers were wash with brine (50 mL×2) and dried over Na₂SO₄, filtered and concentrated under reduced pressure to give a residue. The residue was purified by column chromatography (SiO₂, Petroleum ether/Ethyl acetate=1/0 to 5/1) to give the title compound (961 mg, 83% yield) as a white oil. LC-MS (ESI⁺) m/z 256.2 (M+H)⁺.

Step 9-Ethyl 3-methyl-2-(3-(3-oxopropyl)isoxazol-5-yl)butanoate. To a mixture of ethyl 2-(3-(3-hydroxypropyl)isoxazol-5-yl)-3-methylbutanoate (350 mg, 1.37 mmol) in DCM (5 mL) was added DMP (872 mg, 2.06 mmol) at 0° C. The mixture was stirred at 25° C. for hrs. On completion, the reaction mixture was diluted with NaS₂O₃(100 mL) and adjust pH to 7 used NaHCO₃, then extracted with DCM (100 mL×3). The combined organic layers were wash with brine 100 mL (50 mL×2) and dried over Na₂SO₄, filtered and concentrated under reduced pressure to give the title compound (318 mg) as a white oil. LC-MS (ESI⁺) m/z 254.2 (M+H)⁺.

### 2-((((R)-1-(benzyloxy)-5-((S)-4-(tert-butoxycarbonyl)-3-(methoxycarbonyl)piperazin-1-yl)-3,3-dimethyl-1-oxopentan-2-yl)-12-azaneyl)carbonyl)benzoic acid (Intermediate VV) and 2-((((S)-1-(benzyloxy)-5-((S)-4-(tert-butoxycarbonyl)-3-(methoxycarbonyl)piperazin-1-yl)-3,3-dimethyl-1-oxopentan-2-yl)-12-azaneyl)carbonyl)benzoic acid (Intermediate VW)

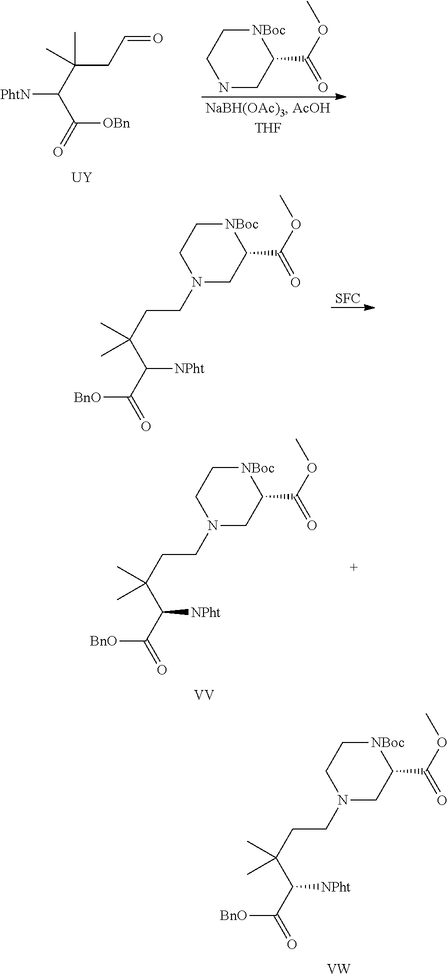

Step 1-(2S)-1-tert-butyl 2-methyl 4-(5-(benzyloxy)-4-(1,3-dioxoisoindolin-2-yl)-3,3-dimethyl-5-oxopentyl)piperazine-1,2-dicarboxylate. To a solution of (S)-1-tert-butyl 2-methyl piperazine-1,2-dicarboxylate (2.41 g, 9.88 mmol, CAS #796096-64-5) and phenyl 2-(1,3-dioxoisoindolin-2-yl)-3,3-dimethyl-5-oxopentanoate (2.5 g, 6.59 mmol, Intermediate UY) in THF (30 mL) was added AcOH (1.58 g, 26.4 mmol, 1.51 mL) at 25° C. and the mixture was stirred for 0.5 hour. Next, NaBH(OAc)₃ (6.98 g, 33.0 mmol) was added at 25° C., then the mixture was stirred at 25° C. for 1 hr. On completion, the reaction mixture was quenched with water (40 mL) and extracted by ethyl acetate (30×4 mL). The extracts were washed by brine (60 mL) and dried over anhydrous sodium sulfate, filtered and concentrated in vacuo to get the crude residue. The residue was purified by column chromatography (SiO₂, Petroleum ether/Ethyl acetate=5/1 to 3:1) to give the title compound (3.5 g, 76% yield) as an off-white oil. LC-MS (ESI⁺) m z 608.3. (M+H)⁺.

Step 2-2-((((R)-1-(benzyloxy)-5-((S)-4-(tert-butoxycarbonyl)-3-(methoxycarbonyl)piperazin-1-yl)-3,3-dimethyl-1-oxopentan-2-yl)-12-azaneyl)carbonyl)benzoic acid and 2-((((S)-1-(benzyloxy)-5-((S)-4-(tert-butoxycarbonyl)-3-(methoxycarbonyl)piperazin-1-yl)-3,3-dimethyl-1-oxopentan-2-yl)-12-azaneyl)carbonyl)benzoic acid. (2S)-1-tert-butyl 2-methyl 4-(3,3-dimethyl-5-oxo-5-phenoxy-4-((phenoxycarbonyl)amino)pentyl)piperazine-1,2-dicarboxylate was purified by SFC (column: REGIS(S,S)WHELK-01(250 mm*25 mm, 10 um); mobile phase: [Neu-MeOH]; B %: 30%-30%, B2.2; 60 min) was separated to give 2-((((R)-1-(benzyloxy)-5-((S)-4-(tert-butoxycarbonyl)-3-(methoxycarbonyl)piperazin-1-yl)-3,3-dimethyl-1-oxopentan-2-yl)-12-azaneyl)carbonyl)benzoic acid (1.5 g, 42% yield) as an off-white oil and 2-((((S)-1-(benzyloxy)-5-((S)-4-(tert-butoxycarbonyl)-3-(methoxycarbonyl)piperazin-1-yl)-3,3-dimethyl-1-oxopentan-2-yl)-12-azaneyl)carbonyl)benzoic acid (1.6 g, 44% yield) as an off-white oil.

### (S)-1-tert-butyl 2-methyl 4-((R)-4-amino-5-(benzyloxy)-3,3-dimethyl-5-oxopentyl)piperazine-1,2-dicarboxylate (Intermediate VX)

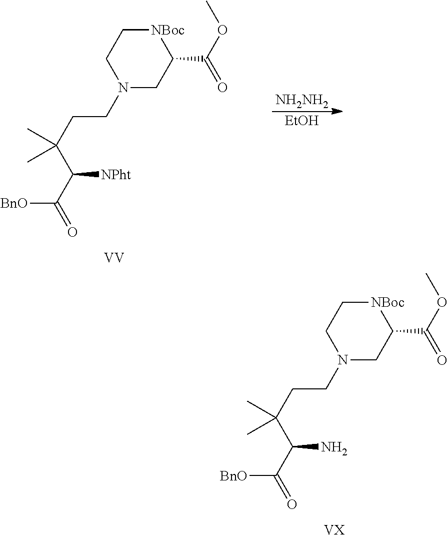

To a solution of (S)-1-tert-butyl 2-methyl 4-((R)-5-(benzyloxy)-4-(1,3-dioxoisoindolin-2-yl)-3,3-dimethyl-5-oxopentyl)piperazine-1,2-dicarboxylate (1.00 g, 1.65 mmol, Intermediate VV) in EtOH (10 mL) was added NH₂NH₂.H₂O (824 mg, 16.5 mmol, 780 uL) at 25° C., then the mixture was stirred at 60° C. for 12 hrs. On completion, the reaction mixture was quenched withy water (20 mL) and extracted with ethyl acetate (30×3 mL). The extracts were washed with brine (50 mL) and dried over anhydrous sodium sulfate, filtered and concentrated in vacuo to give the title compound (900 mg) as an off-white oil. LC-MS (ESI⁺) m/z 478.2. (M+H)⁺.

### (R)-5-((S)-4-(tert-butoxycarbonyl)-3-(methoxycarbonyl)piperazin-1-yl)-3,3-dimethyl-2-((phenoxycarbonyl)amino)pentanoic acid (Intermediate VY)

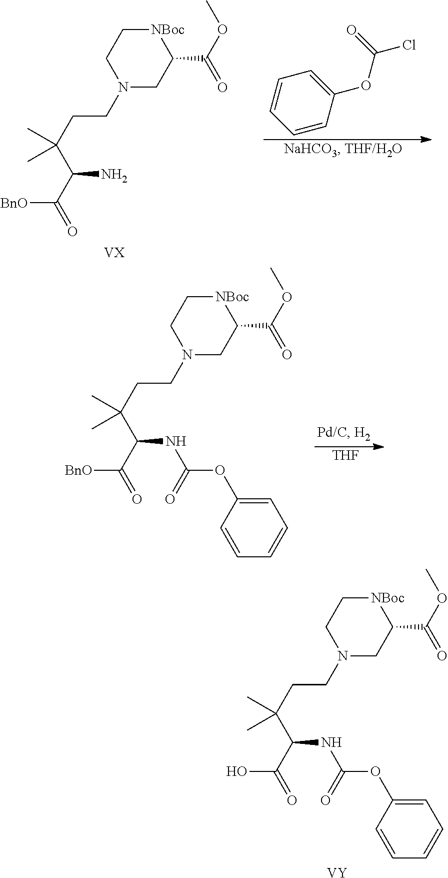

Step 1-(S)-1-tert-butyl 2-methyl 4-((R)-5-(benzyloxy)-3,3-dimethyl-5-oxo-4-((phenoxycarbonyl)amino)pentyl)piperazine-1,2-dicarboxylate. To a solution of (S)-1-tert-butyl 2-methyl 4-((R)-4-amino-5-(benzyloxy)-3,3-dimethyl-5-oxopentyl)piperazine-1,2-dicarboxylate (900 mg, 1.88 mmol, Intermediate VX) in THF (8 mL) and H₂O (4 mL) was added NaHCO₃ (633 mg, 7.54 mmol, 293 uL) at 25° C., then phenyl carbonochloridate (443 mg, 2.83 mmol, 354 uL) was added at 0° C., then the mixture was stirred at 25° C. for 0.5 hour. On completion, the reaction mixture was quenched by water (20 mL) and extracted by ethyl acetate (20×4 mL). The extracts were washed by brine (30 mL) and dried over anhydrous sodium sulfate, filtered and concentrated in vacuo to get the crude residue. The residue was purified by column chromatography (SiO₂, Petroleum ether/Ethyl acetate=5/1 to 3/1) to give the title compound (950 mg, 73% yield) as an off-white oil. LC-MS (ESI⁺) m/z 598.9. (M+H)⁺.

Step 2-(R)-5-((S)-4-(tert-butoxycarbonyl)-3-(methoxycarbonyl)piperazin-1-yl)-3,3-dimethyl-2-((phenoxycarbonyl)amino)pentanoic acid. To a solution of (S)-1-tert-butyl 2-methyl 4-((R)-5-(benzyloxy)-3,3-dimethyl-5-oxo-4-((phenoxycarbonyl)amino)pentyl)piperazine-1,2-dicarboxylate (900 mg, 1.51 mmol) in THF (7 mL) was added Pd/C (700 mg, 1.51 mmol, 60 wt %) at 25° C., then the mixture was stirred at 25° C. for 12 hrs. On completion, the reaction was dissolved in THF and filtered to remove the insolubles. The filter liquor was concentrated in vacuo to give the title compound (700 mg) as an off-white oil. LC-MS (ESI⁺) m/z 508.3. (M+H)⁺.

### (S)-1-tert-butyl 2-methyl 4-((R)-5-((2S,4R)-2-((4-ethynylbenzyl)carbamoyl)-4-hydroxypyrrolidin-1-yl)-3,3-dimethyl-5-oxo-4-((phenoxycarbonyl)amino)pentyl)piperazine-1,2-dicarboxylate (Intermediate VZ)

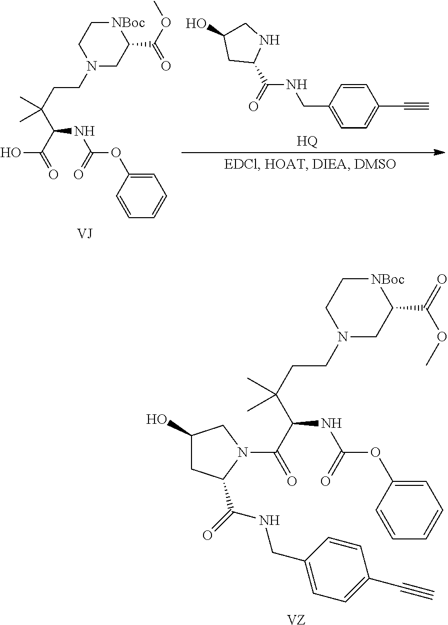

To a solution of (R)-5-((S)-4-(tert-butoxycarbonyl)-3-(methoxycarbonyl)piperazin-1-yl)-3,3-dimethyl-2-((phenoxycarbonyl)amino)pentanoic acid (400 mg, 788 umol, Intermediate VY) and (2S,4R)—N-(4-ethynylbenzyl)-4-hydroxypyrrolidine-2-carboxamide (192 mg, 788 umol, Intermediate HQ) in DMSO (1 mL) was added EDCI (227 mg, 1.18 mmol), HOAt (161 mg, 1.18 mmol, 165 uL) and DIEA (509 mg, 3.94 mmol, 686 uL) at 25° C., then the mixture was stirred at 25° C. for 1 hr. On completion, the reaction mixture was quenched with water (20 mL) and extracted by ethyl acetate/dichloromethane (30×3 mL). The extracts were washed with brine (50 mL) and dried over anhydrous sodium sulfate, filtered and concentrated in vacuo to get the crude residue. The residue was purified by column chromatography (SiO₂, Petroleum ether/Ethyl acetate=0/1) to give the title compound (400 mg, 67% yield) as an off-white oil. LC-MS (ESI⁺) m/z 734.4. (M+H)⁺.

### (S)-1-tert-butyl 2-methyl 4-((S)-5-(benzyloxy)-4-(1,3-dioxoisoindolin-2-yl)-3,3-dimethyl-5-oxopentyl)piperazine-1,2-dicarboxylate (Intermediate WA)

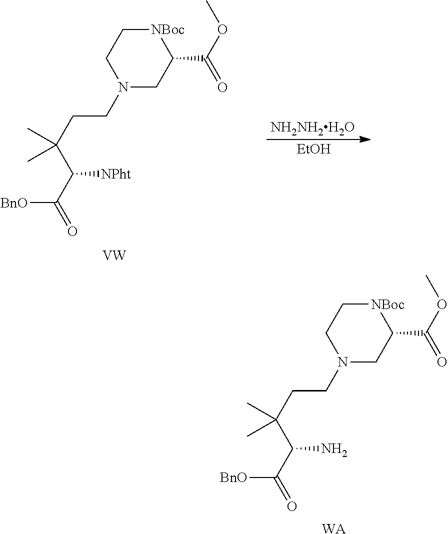

To a solution of 2-((((S)-1-(benzyloxy)-5-((S)-4-(tert-butoxycarbonyl)-3-(methoxycarbonyl)piperazin-1-yl)-3,3-dimethyl-1-oxopentan-2-yl)-12-azaneyl)carbonyl)benzoic acid (1.00 g, 1.65 mmol, Intermediate VW) in EtOH (10 mL) was added NH₂NH₂.H₂O (824 mg, 16.5 mmol, 780 uL, 85% solution) at 25° C., then the mixture was stirred at 25° C. for 12 hrs. On completion, the reaction mixture was quenched with water (20 mL) and extracted with ethyl acetate (30×3 mL). The extracts were washed with brine (50 mL) and dried over anhydrous sodium sulfate, filtered and concentrated in vacuo to give the title compound (780 mg) as an off-white oil. LC-MS (ESI⁺) m/z 478.4. (M+H)⁺.

### (S)-5-((S)-4-(tert-butoxycarbonyl)-3-(methoxycarbonyl)piperazin-1-yl)-3,3-dimethyl-2-((phenoxycarbonyl)amino)pentanoic acid (Intermediate WB)

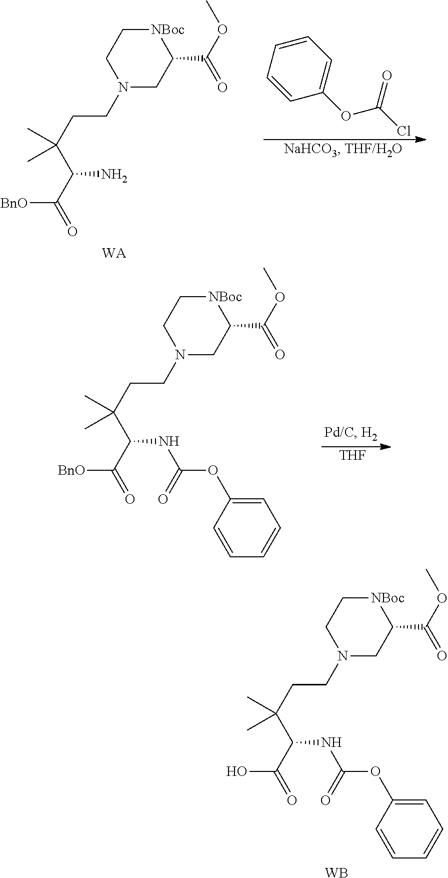

Step 1-(S)-1-tert-butyl 2-methyl 4-((S)-5-(benzyloxy)-3,3-dimethyl-5-oxo-4-((phenoxycarbonyl)amino)pentyl)piperazine-1,2-dicarboxylate. To a solution of (S)-1-tert-butyl 2-methyl 4-((S)-5-(benzyloxy)-4-(1,3-dioxoisoindolin-2-yl)-3,3-dimethyl-5-oxopentyl)piperazine-1,2-dicarboxylate (780 mg, 1.63 mmol, Intermediate WA) in THF (6 mL) and H₂O (3 mL) was added NaHCO₃ (137 mg, 1.63 mmol, 63.5 uL) at 25° C., then phenyl carbonochloridate (384 mg, 2.45 mmol) was added at 0° C., then the mixture was stirred at 25° C. for 0.5 hour. On completion, the reaction mixture was quenched with water (20 mL) and extracted by ethyl acetate/dichloromethane (20×4 mL). The extracts were washed with brine (30 mL) and dried over anhydrous sodium sulfate, filtered and concentrated in vacuo to get the crude residue. The residue was purified by column chromatography (SiO₂, Petroleum ether/Ethyl acetate=5/1 to 3/1) to give the title compound (780 mg) as an off-white oil. LC-MS (ESI⁺) m/z 599.1. (M+H)⁺.

Step 2-(S)-5-((S)-4-(tert-butoxycarbonyl)-3-(methoxycarbonyl)piperazin-1-yl)-3,3-dimethyl-2-((phenoxycarbonyl)amino)pentanoic acid. To a solution of (S)-1-tert-butyl 2-methyl 4-((S)-5-(benzyloxy)-3,3-dimethyl-5-oxo-4-((phenoxycarbonyl)amino)pentyl)piperazine-1,2-dicarboxylate (700 mg, 1.17 mmol) in THF (8 mL) was added Pd/C (900 mg, 1.17 mmol, 60 et %) under N₂ atmosphere. The suspension was degassed and purged with H₂ three times. The mixture was then stirred under H₂ (15 Psi) at 25° C. for 12 hrs. On completion, the resulting product was dissolved in THF and filtered to remove the insolubles. The filter liquor was concentrated in vacuo to give the title compound (600 mg) as an off-white oil. LC-MS (ESI⁺) m/z 508.3. (M+H)⁺.

### (S)-1-tert-butyl 2-methyl 4-((S)-5-((2S,4R)-2-((4-ethynylbenzyl)carbamoyl)-4-hydroxypyrrolidin-1-yl)-3,3-dimethyl-5-oxo-4-((phenoxycarbonyl)amino)pentyl)piperazine-1,2-dicarboxylate (Intermediate WC)

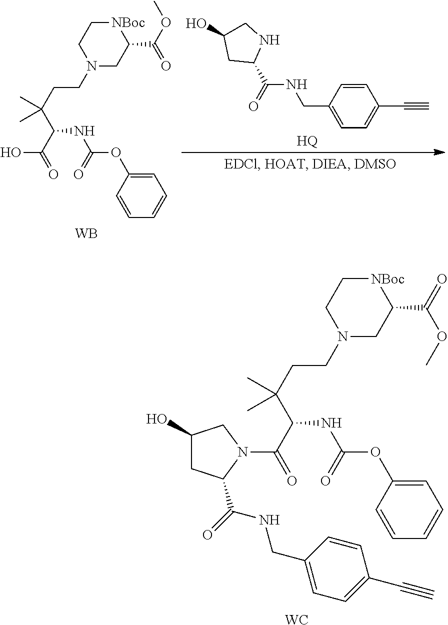

To a solution of (S)-5-((S)-4-(tert-butoxycarbonyl)-3-(methoxycarbonyl)piperazin-1-yl)-3,3-dimethyl-2-((phenoxycarbonyl)amino)pentanoic acid (210 mg, 414 umol, Intermediate WB) in DMSO (5 mL) was added EDCI (119 mg, 621 umol), HOAt (84.5 mg, 621 umol, 86.8 uL), DIEA (267 mg, 2.07 mmol, 360 uL) and (2S,4R)—N-(4-ethynylbenzyl)-4-hydroxypyrrolidine-2-carboxamide (80.9 mg, 331 umol, Intermediate HQ) at 25° C., then the mixture was stirred at 25° C. for 1 hr. On completion, the reaction mixture was quenched with water (20 mL) and extracted by ethyl acetate (20×3 mL). The extracts were washed with brine (50 mL) and dried over anhydrous sodium sulfate, filtered and concentrated in vacuo to get the crude residue. The residue was purified by column chromatography (SiO₂, Petroleum ether/Ethyl acetate=0/1) to give the title compound (250 mg, 75% yield) as an off-white oil. LC-MS (ESI⁺) m/z 734.8. (M+H)⁺.

### (4-Ethynyl-3,5-difluorophenyl)methanamine (Intermediate YG)

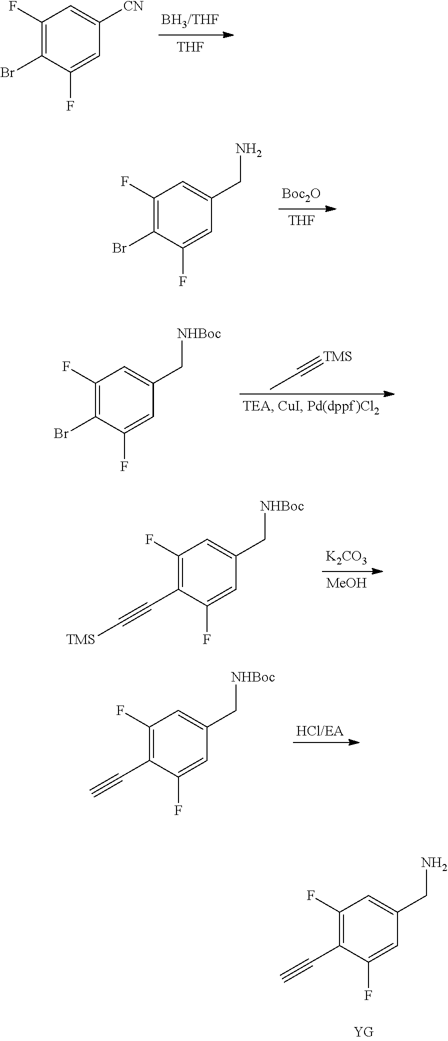

Step 1-(4-Bromo-3,5-difluorophenyl)methanamine. To a solution of 4-bromo-3,5-difluoro-benzonitrile (5 g, 22.9 mmol, CAS #123688-59-5) in THF (50 mL) was added BH₃THF (1 M, 57.3 mL) at 0° C. under N₂ atmosphere. The mixture was stirred at 50° C. for 2 hrs. On completion, the reaction mixture was quenched with 1 M HCl until the pH=4. Then NaOH aqueous solution was added until the pH=10 and the mixture was extracted with DCM (40 mL×3). The combined organic layers were washed with brine (60 mL×2), dried over Na₂SO₄, filtered and concentrated under reduced pressure to give the title compound (5 g) as a yellow oil. LC-MS (ESI⁺) m/z 204.9 (M-NH₂)⁺.

Step 2-Tert-butyl 4-bromo-3,5-difluorobenzylcarbamate. To a solution of (4-bromo-3,5-difluoro-phenyl)methanamine (5 g, 22.5 mmol) in THF (50 mL) was added Boc₂O (9.83 g, 45.0 mmol). The mixture was stirred at 30° C. for 12 hrs. On completion, the reaction mixture was quenched with NH₄Cl (50 mL) at 25° C., and then extracted with EA (50 mL×3). The combined organic layers were washed with NaCl (50 mL×3), dried over Na₂SO₄, filtered and concentrated under reduced pressure to give a residue. The residue was purified by column chromatography (SiO₂, Petroleum ether/Ethyl acetate=50/1 to 10/1) to give the title compound (2.5 g, 29% yield) as a white solid. LC-MS (ESI⁺) m/z 265.8 (M−56)⁺.

Step 3-Tert-butyl 3,5-difluoro-4-((trimethylsilyl)ethynyl)benzylcarbamate. To a solution of tert-butyl N-[(4-bromo-3,5-difluoro-phenyl)methyl]carbamate (0.5 g, 1.55 mmol) in DMF (5 mL) was added TEA (2.36 g, 23.2 mmol), CuI (59.1 mg, 310 umol), Pd(PPh₃)₂Cl₂ (109 mg, 155 umol) and ethynyl(trimethyl)silane (1.52 g, 15.5 mmol). The mixture was stirred at 110° C. for 2 hrs under microwave. On completion, the reaction mixture was quenched with NH4C1 (10 mL) at 25° C., and then extracted with EA (10 mL×3). The combined organic layers were washed with NaCl (10 mL×3), dried over Na₂SO₄, filtered and concentrated under reduced pressure to give a residue. The crude product was purified by reversed-phase HPLC (0.1% FA condition) to give the title compound (0.18 g, 31% yield, FA) as a white solid. LC-MS (ESI⁺) m/z 283.8 (M−56)⁺.

Step 4-Tert-butyl 4-ethynyl-3,5-difluorobenzylcarbamate. To a solution of tert-butyl N-[[3,5-difluoro-4-(2-trimethylsilylethynyl)phenyl]methyl]carbamate (0.16 g, 471 umol, FA) in MeOH (2 mL) was added K₂CO₃ (65.1 mg, 471 umol). The mixture was stirred at 25° C. for 0.5 hrs. On completion, the reaction mixture was quenched with NH₄Cl 5 mL at 25° C., and then extracted with EA (5 mL×3). The combined organic layers were washed with NaCl (5 mL×3), dried over Na₂SO₄, filtered and concentrated under reduced pressure to give a residue. The crude product was purified by reversed-phase HPLC (0.1% FA condition) to give the title compound (90 mg, 71% yield, FA) as a yellow solid. LC-MS (ESI⁺) m/z 212.1 (M−56)⁺.

Step 5-(4-Ethynyl-3,5-difluorophenyl)methanamine. To a solution of tert-butyl N-[(4-ethynyl-3,5-difluoro-phenyl)methyl]carbamate (50 mg, 187 umol, FA) in DCM (0.5 mL) was added HCl/dioxane (4 M, 0.1 mL). The mixture was stirred at 25° C. for 1 hrs. On completion, the reaction mixture was concentrated under reduced pressure to give the title compound (40 mg, HCl) as a yellow solid. LC-MS (ESI+) m/z 150.8 (M−NH₂)⁺.

### (R)-2-(6-(5-(1-(2-azaspiro[3.3]heptan-6-ylmethyl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate YH)

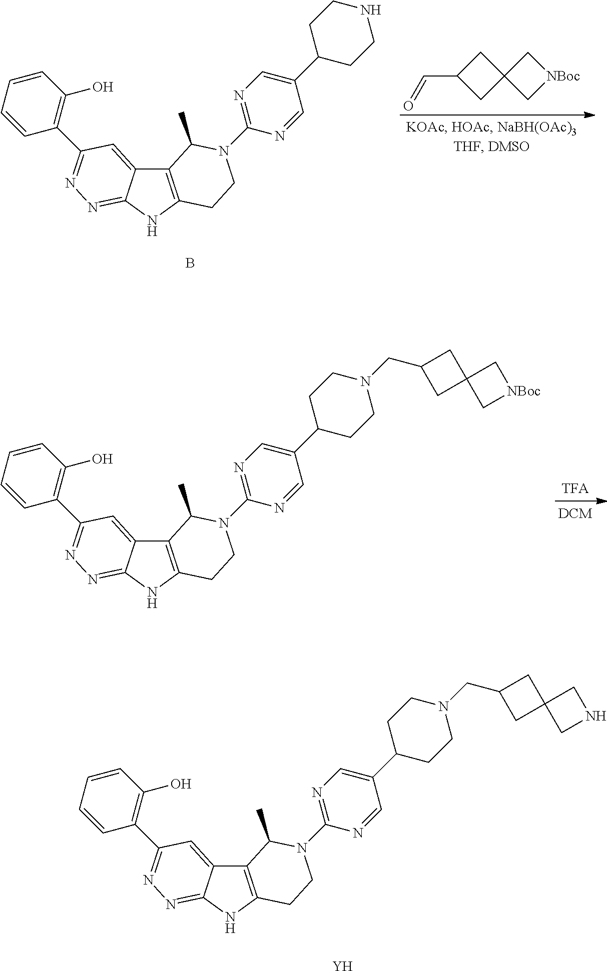

Step 1-(R)-tert-butyl 6-((4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)methyl)-2-azaspiro[3.3]heptane-2-carboxylate. To a solution of 2-[(3R)-3-methyl-4-[5-(4-piperidyl)pyrimidin-2-yl]-4,8,10,11-tetrazatricyclo[7.4.0.0^{2,7}]trideca-1(13),2(7),9,11-tetraen-12-yl]phenol (500 mg, 1.13 mmol, Intermediate B) in THF (4 mL) and DMSO (4 mL) was added KOAc (333 mg, 3.40 mmol) and the mixture was stirred at 25° C. for 0.5 hr. Then HOAc (204 mg, 3.40 mmol) and tert-butyl 6-formyl-2-azaspiro[3.3]heptane-2-carboxylate (255 mg, 1.13 mmol, CAS #1440960-67-7) were added and the mixture was stirred at 25° C. for 0.5 hr. Next, NaBH(OAc)₃ (600 mg, 2.83 mmol) was added at 0° C. and the mixture was stirred at 25° C. for 3 hrs. On completion, the reaction mixture was concentrated under reduced pressure to remove THF. The crude product was purified by reversed-phase HPLC (0.1% FA condition) to give the title compound (140 mg, 17% yield, FA) as a yellow solid. LC-MS (ESI+) m/z 651.7 (M+H)⁺.

Step 2-(R)-2-(6-(5-(1-(2-azaspiro[3.3]heptan-6-ylmethyl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 6-[[4-[2-[(3R)-12-(2-hydroxyphenyl)-3-methyl-4,8,10,11-tetrazatricyclo[7.4.0.0^{2,7}]trideca-1(13),2(7),9,11-tetraen-4-yl]pyrimidin-5-yl]-1-piperidyl]methyl]-2-azaspiro[3.3]heptane-2-carboxylate (140 mg, 200 umol) in DCM (3 mL) was added TFA (0.6 mL). The mixture was stirred at 25° C. for 1 hr. On completion, the reaction mixture was concentrated under reduced pressure to remove dichloromethane. The crude product was purified by reversed-phase HPLC (0.1% FA condition) to give the title compound (120 mg, 92% yield, FA) as a yellow solid. LC-MS (ESI+) m/z 551.1 (M+H)⁺.

### Tert-butyl 6-(2-oxoethyl)-2-azaspiro[3.3]heptane-2-carboxylate (Intermediate YS)

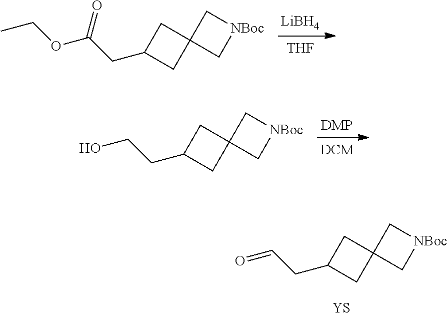

Step 1-Tert-butyl 6-(2-hydroxyethyl)-2-azaspiro[3.3]heptane-2-carboxylate. To a solution of tert-butyl 6-(2-ethoxy-2-oxo-ethyl)-2-azaspiro[3.3]heptane-2-carboxylate (200 mg, 705 umol, CAS #2173992-27-1) in THF (2 mL) was added LiBH₄ (33.8 mg, 1.55 mmol) at 0° C. under N₂ atmosphere. The mixture was stirred at 25° C. for 2 hrs. On completion, the reaction mixture was quenched with NH₄Cl (10 mL) at 0° C., then extracted with EA (20 mL×2). The combined organic layers were washed with brine (10 mL×2), dried over Na₂SO₄, filtered and concentrated under reduced pressure to give the title compound (150 mg) as a yellow oil. ¹H NMR (400 MHz, CHLOROFORM-d) 6=4.11 (q, J=7.2 Hz, 2H), 3.94-3.92 (m, 2H), 3.82-3.79 (m, 2H), 3.62 (s, 1H), 3.58 (t, J=6.4 Hz, 1H), 2.53 (m, 1H), 2.59 (s, 1H), 2.39-2.29 (m, 3H), 2.28-2.19 (m, 1H), 2.05 (s, 1H), 1.87 (d, J=2.0 Hz, 2H), 1.68-1.58 (m, 1H), 1.43 (s, 9H), 1.24 (t, J=7.2 Hz, 3H).

Step 2-Tert-butyl 6-(2-oxoethyl)-2-azaspiro[3.3]heptane-2-carboxylate. To a solution of tert-butyl 6-(2-hydroxyethyl)-2-azaspiro[3.3]heptane-2-carboxylate (800 mg, 3.32 mmol) in DCM (25 mL) was added DMP (1.69 g, 3.98 mmol) at 0° C. The mixture was stirred at 0-25° C. for 1 hr. On completion, the reaction mixture was quenched with Na₂S₂O₃ (5 mL) at 0° C., and then diluted with NaHCO₃ to adjust the pH to 8, then extracted with DCM (10 mL×5). The combined organic layers were dried over Na₂SO₄, filtered and concentrated under reduced pressure to give a residue. The residue was purified by column chromatography (SiO₂, Petroleum ether/Ethyl acetate=10/1 to 4/1) to give the title compound (762 mg, 87% yield) as a colorless oil. ¹H NMR (400 MHz, CHLOROFORM-d) 6=9.63 (t, J=1.6 Hz, 1H), 3.88-3.85 (m, 2H), 3.74 (s, 2H), 2.58-2.49 (m, 1H), 2.49-2.43 (m, 2H), 2.34-2.24 (m, 2H), 1.98 (s, 2H), 1.36 (s, 9H).

### (R)-2-(6-(5-(1-(2-(2-azaspiro[3.3]heptan-6-yl)ethyl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol (Intermediate YT)

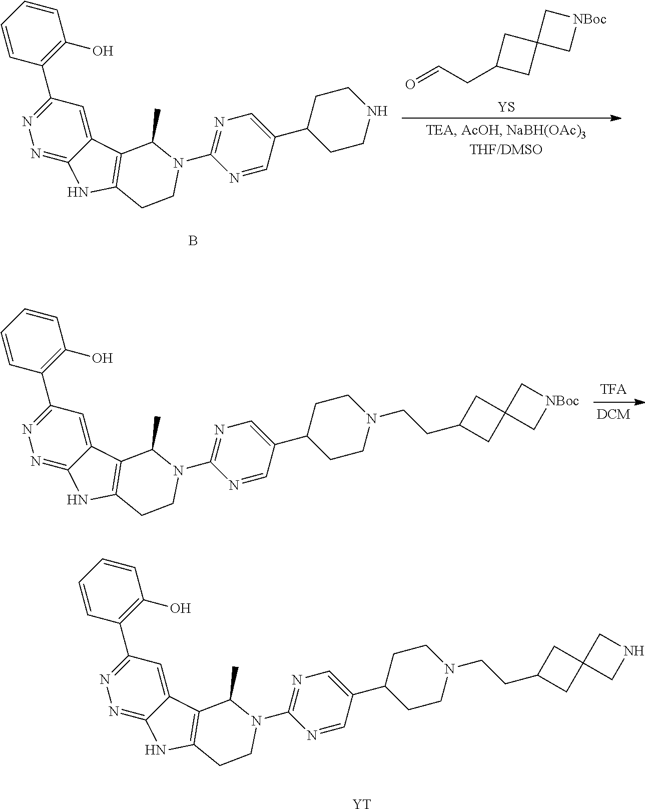

Step 1-(R)-tert-butyl 6-(2-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)ethyl)-2-azaspiro[3.3]heptane-2-carboxylate. To a solution of 2-[(3R)-3-methyl-4-[5-(4-piperidyl)pyrimidin-2-yl]-4,8,10,11-tetrazatricyclo[7.4.0.02,7]trideca-1(13),2(7),9,11-tetraen-12-yl]phenol (300 mg, 628 umol, HCl, Intermediate B) in THF (3 mL) and DMSO (2 mL) was added TEA (159 mg, 1.57 mmol) and the mixture was stirred for 30 mins. Then tert-butyl 6-(2-oxoethyl)-2-azaspiro[3.3]heptane-2-carboxylate (300 mg, 1.26 mmol, Intermediate YS) and AcOH (94.2 mg, 1.57 mmol) and stirred for 1.5 hrs. Next, NaBH(OAc)₃ (399 mg, 1.88 mmol) was added at 0° C. The mixture was stirred at 0-25° C. for 1 hr. On completion, the reaction mixture was filtered and concentrated under reduced pressure to give a residue. The residue was purified by prep-HPLC (FA condition) to give the title compound (210 mg, 46% yield, FA) as a yellow solid. LC-MS (ESI+) m/z 665.6 (M+H)⁺.

Step 2-(R)-2-(6-(5-(1-(2-(2-azaspiro[3.3]heptan-6-yl)ethyl)piperidin-4-yl)pyrimidin-2-yl)-5-methyl-6,7,8,9-tetrahydro-5H-pyrido[3′,4′:4,5]pyrrolo[2,3-c]pyridazin-3-yl)phenol. To a solution of tert-butyl 6-[2-[4-[2-[(3R)-12-(2-hydroxyphenyl)-3-methyl-4,8,10,11-tetrazatricyclo[7.4.0.02,7]trideca-1(13),2(7),9,11-tetraen-4-yl]pyrimidin-5-yl]-1-piperidyl]ethyl]-2-azaspiro[3.3]heptane-2-carboxylate (210 mg, 316 umol) in DCM (2 mL) was added TFA (616 mg, 5.40 mmol). The mixture was stirred at 25° C. for 1 hr. On completion, the reaction mixture was filtered and concentrated under reduced pressure to give a residue. The residue was purified by prep-HPLC (FA condition) to give the title compound (160 mg, 81% yield, FA) as a yellow solid. LC-MS (ESI+) m/z 565.6 (M+H)⁺.

### (2S,4R)-1-((S)-2-amino-5-hydroxy-3,3-dimethylpentanoyl)-N—((S)-1-(4-ethynylphenyl)ethyl)-4-hydroxypyrrolidine-2-carboxamide (Intermediate YY)

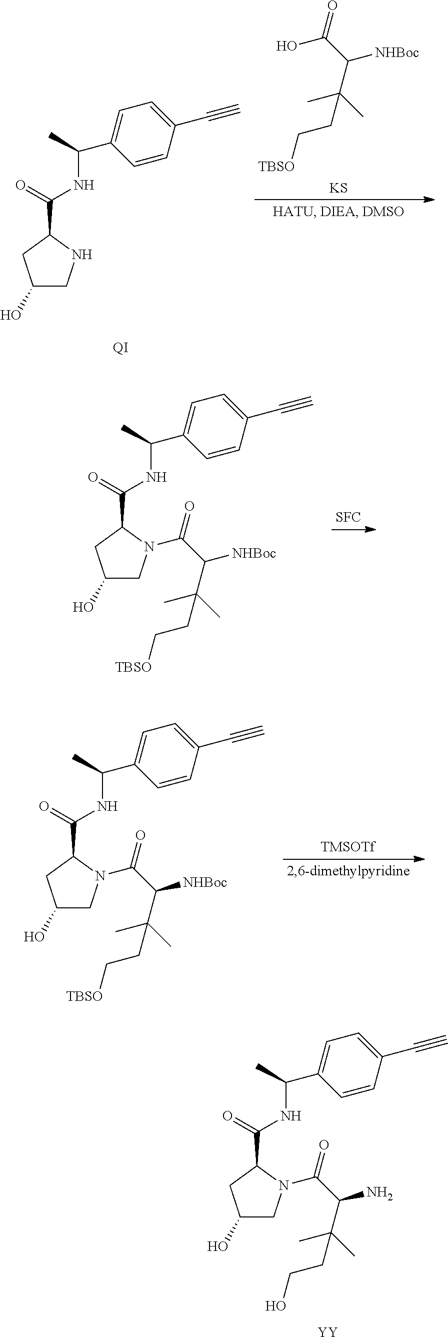

Step 1-Tert-butyl (5-((tert-butyldimethylsilyl)oxy)-1-((2S,4R)-2-(((S)-1-(4-ethynylphenyl)ethyl)carbamoyl)-4-hydroxypyrrolidin-1-yl)-3,3-dimethyl-1-oxopentan-2-yl)carbamate. To a solution of 2-(tert-butoxycarbonylamino)-5-[tert-butyl(dimethyl)silyl]oxy-3,3-dimethyl-pentanoic acid (2 g, 5.33 mmol, Intermediate KS), (2S,4R)—N-[(1S)-1-(4-ethynylphenyl)ethyl]-4-hydroxy-pyrrolidine-2-carboxamide (2.04 g, 6.92 mmol, HCl, Intermediate QI) in DMSO (20 mL) was added HATU (3.04 g, 7.99 mmol) and DIEA (2.06 g, 15.9 mmol). The mixture was stirred at 25° C. for 1 hr. On completion, the reaction mixture was extracted with EA (30 mL×3). The combined organic layers were washed with brine 40 (20 mL×2), dried over Na₂SO₄, filtered and concentrated under reduced pressure to give a residue. The residue was purified by prep-HPLC (NH₃.H₂O condition) to give the title compound (1 g, 28% yield) as a white solid. LC-MS (ESI⁺) m/z 616.2 (M−H)⁺.

Step 2-Tert-butyl ((S)-5-((tert-butyldimethylsilyl)oxy)-1-((2S,4R)-2-(((S)-1-(4-ethynylphenyl)ethyl)carbamoyl)-4-hydroxypyrrolidin-1-yl)-3,3-dimethyl-1-oxopentan-2-yl)carbamate. The residue was separated by prep-HPLC (column: DAICEL CHIRALPAK IC (250 mm*30 mm, 10 um); mobile phase: [0.1% NH₃H₂O IPA]; B %: 20%-20%, A3.4; 32 min) to give the title compound (750 mg, 40% yield) as a white solid. LC-MS (ESI⁺) m/z 616.3 (M−H)⁺.

Step 3-(2S,4R)-1-((S)-2-amino-5-hydroxy-3,3-dimethylpentanoyl)-N—((S)-1-(4-ethynylphenyl)ethyl)-4-hydroxypyrrolidine-2-carboxamide. To a solution of tert-butyl N-[(1S)-4-[tert-butyl(dimethyl)silyl]oxy-1-[(2S,4R)-2-[[(1S)-1-(4-ethynylphenyl)ethyl]carbamoyl]-4-hydroxy-pyrrolidine-1-carbonyl]-2,2-dimethyl-butyl]carbamate (730 mg, 1.19 mmol) in DCM (7 mL) was added TMSOTf (790 mg, 3.56 mmol) and 2,6-dimethyl(1¹⁵N)pyridine (640 mg, 5.93 mmol). The mixture was stirred at 25° C. for 1 hr. On completion, the reaction mixture was filtered and concentrated under reduced pressure to give a residue. The residue was purified by prep-HPLC (FA condition) to give the title compound (520 mg, 97% yield, FA) as a white solid. LC-MS (ESI⁺) m/z 402.2 (M−H)⁺.

### Phenyl ((S)-1-((2S,4R)-2-(((S)-1-(4-ethynylphenyl)ethyl)carbamoyl)-4-hydroxypyrrolidin-1-yl)-5-hydroxy-3,3-dimethyl-1-oxopentan-2-yl)carbamate (Intermediate YZ)

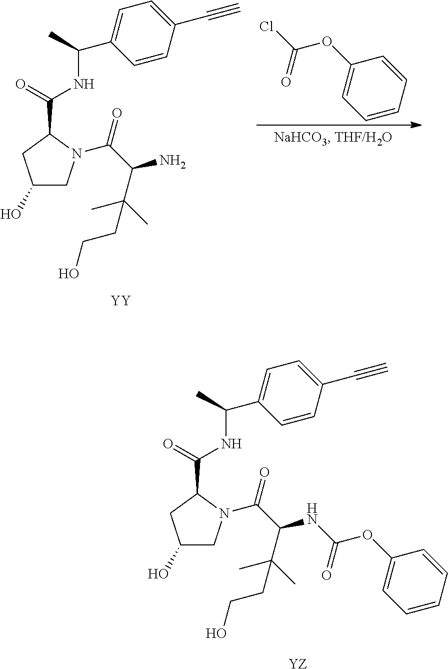

To a solution of (2S,4R)-1-[(2S)-2-amino-5-hydroxy-3,3-dimethyl-pentanoyl]-N-[(1S)-1-(4-ethynylphenyl)ethyl]-4-hydroxy-pyrrolidine-2-carboxamide (450 mg, 1.12 mmol, Intermediate YY) in THF (10 mL) and H₂O (2 mL) was added NaHCO₃ (282 mg, 3.36 mmol) and phenyl carbonochloridate (210 mg, 1.34 mmol). The mixture was stirred at 0-25° C. for 1 hr. On completion, the reaction mixture was adjusted the pH to 7 with NH₄Cl (20 ml) and extracted with DCM (50 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=1/5 to DCM: MeOH=15:1) to give the title compound (500 mg, 84% yield) as a white solid. LC-MS (ESI⁺) m/z 522.5 (M−H)⁺.

### Examples 1 (Method 1): Syntheses of (2S,4R)-4-hydroxy-1-(2-(3-(((1R,4S)-4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)methoxy)isoxazol-5-yl)-3-methylbutanoyl)-N-(4-(4-methylthiazol-5-yl)benzyl)pyrrolidine-2-carboxamide (I-1) and (2S,4R)-4-hydroxy-1-(2-(3-(((1S,4R)-4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)methoxy)isoxazol-5-yl)-3-methylbutanoyl)-N-(4-(4-methylthiazol-5-yl)benzyl)pyrrolidine-2-carboxamide (I-2)

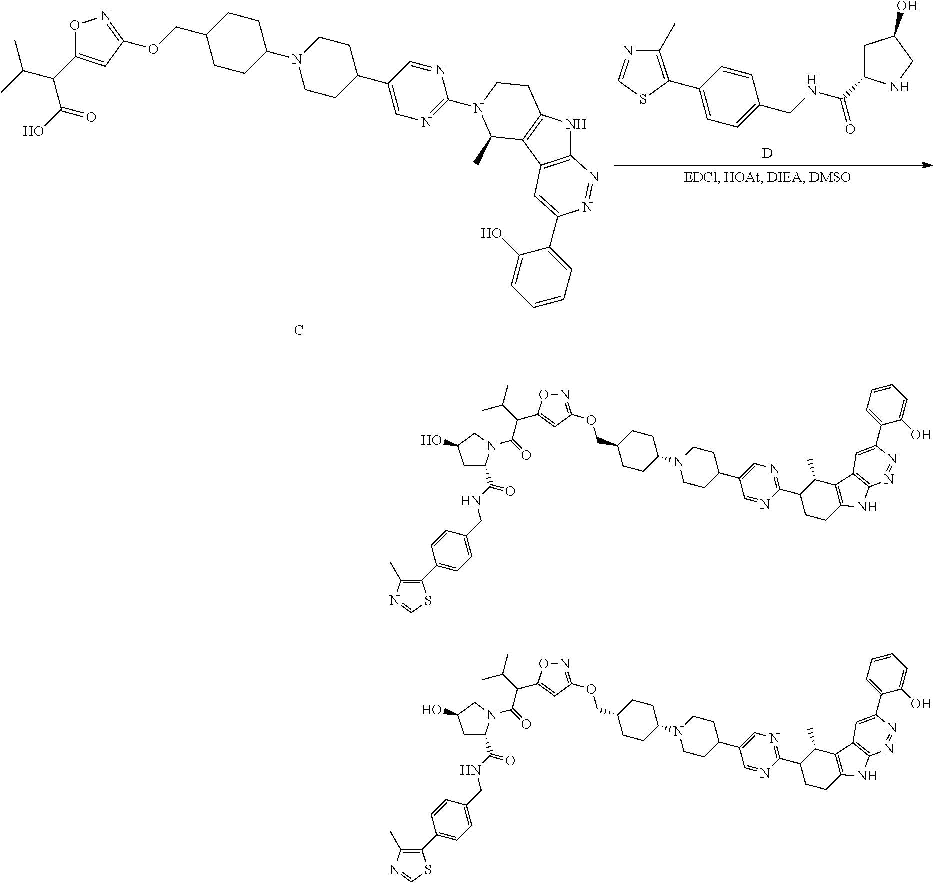

To a solution of 2-[3-[[4-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-cyclohexyl]methoxy]isoxazol-5-yl]-3-methyl-butanoic acid (40.0 mg, 55.4 umol, Intermediate C) in DMSO (1 mL) was added HOAt (11.3 mg, 83.2 umol), DIEA (21.5 mg, 166 umol) and EDCI (15.9 mg, 83.2 umol) and the mixture was stirred for 30 minutes at 40° C. Then (2S,4R)-4-hydroxy-N-[[4-(4-methylthiazol-5-yl)phenyl]methyl]pyrrolidine-2-carboxamide (39.3 mg, 111 umol, HCl, Intermediate D) was added and the mixture was stirred at 40° C. for 3.5 hours. On completion, the reaction mixture was filtered and concentrated under reduced pressure to give a residue. The residue was purified by prep-HPLC (neutral condition column: Waters xbridge 150*25 mm 10 um; mobile phase: [water(10 mM NH₄HCO₃)-ACN]; B %: 42%-72%, 11 min) to give (2S,4R)-4-hydroxy-1-(2-(3-(((1S,4R)-4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)methoxy)isoxazol-5-yl)-3-methylbutanoyl)-N-(4-(4-methylthiazol-5-yl)benzyl)pyrrolidine-2-carboxamide (21.7 mg, 39% yield) as a yellow solid (¹H NMR (400 MHz, DMSO-d₆) δ 12.43 (br s, 1H) 8.96-9.02 (m, 1H) 8.72 (s, 1H) 8.45-8.56 (m, 1H) 8.33 (d, J=4.0 Hz, 2H) 8.21 (dd, J=8.0 Hz, 4.0 Hz, 1H) 7.37-7.45 (m, 3H) 7.27-7.34 (m, 2H) 6.94-6.98 (m, 2H) 6.02-6.12 (m, 2H) 5.13 (m, 1H) 5.05 (br dd, J=16.0, 4.0 Hz, 1H) 4.37-4.48 (m, 1H) 4.27-4.37 (m, 3H) 4.01-4.11 (dd, J=28.0 Hz, 4.0 Hz, 2H) 3.71-3.81 (m, 1H) 3.57-3.68 (m, 1H) 3.38-3.49 (m, 2H) 2.90-3.02 (m, 4H) 2.45 (d, J=4.0 Hz, 2H) 2.43 (d, J=8.0 Hz, 1H) 2.30-2.36 (m, 1H) 2.21-2.28 (m, 2H) 2.02-2.12 (m, 3H) 1.89-1.93 (m, 1H) 1.71 (m, 2H) 1.38-1.66 (m, 15H) 0.96 (t, J=8.0 Hz, 3H) 0.80-0.85 (dd, J=12.0 Hz, 8.0 Hz, 3H). LC-MS (ESI⁺) m/z 1020.8 (M+H)⁺. (2S,4R)-4-hydroxy-1-(2-(3-(((1R,4S)-4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)methoxy)isoxazol-5-yl)-3-methylbutanoyl)-N-(4-(4-methylthiazol-5-yl)benzyl)pyrrolidine-2-carboxamide was further purified by prep-HPLC (FA condition column: Phenomenex luna C18 150*25 mm*10 um; mobile phase: [water(0.225% FA)-ACN]; B %: 15%-45%, 10 min). to give (2S,4R)-4-hydroxy-1-(2-(3-(((1R,4S)-4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)methoxy)isoxazol-5-yl)-3-methylbutanoyl)-N-(4-(4-methylthiazol-5-yl)benzyl)pyrrolidine-2-carboxamide (21.1 mg, 36% yield, FA) as a yellow solid. ¹H NMR (400 MHz, DMSO-d₆) δ 12.45 (s, 1H) 8.97-8.99 (m, 1H) 8.72 (s, 1H) 8.45-8.56 (m, 1H) 8.33 (s, 2H) 8.20-8.24 (m, 2H) 7.36-7.48 (m, 3H) 7.28-7.35 (m, 2H) 6.94-7.00 (m, 2H) 6.02-6.11 (m, 2H) 5.03-5.08 (dd, J=12.0 Hz, 4.0 Hz, 1H) 4.25-4.46 (m, 4H) 3.88-3.96 (m, 3H) 3.72-3.81 (m, 2H) 3.66 (d, J=12.0 Hz, 1H) 3.37-3.46 (m, 4H) 2.88-2.97 (m, 4H) 2.46 (s, 2H) 2.42 (d, J=4.0 Hz, 1H) 2.22-2.38 (m, 5H) 1.99-2.08 (m, 1H) 1.87-1.95 (m, 1H) 1.81-1.86 (m, 2H) 1.69-1.80 (m, 4H) 1.59-1.67 (m, 2H) 1.54 (d, J=12.0 Hz, 3H) 1.21-1.33 (m, 2H) 1.02-1.10 (m, 1H) 0.96 (t, J=8.0 Hz, 3H) 0.75-0.88 (m, 3H). LC-MS (ESI⁺) m/z 1020.8 (M+H)⁺. Absolute stereochemistry was assigned arbitrarily.

TABLE 3
Compounds synthesized via Method 1 using the corresponding amines and acids for the coupling.

| | | | LCMS (ESI+) | |
|---|---|---|---|---|
| I-#^{a} | Amine | Acid | m/z (M + H)⁺ | ¹H NMR (400 MHz, DMSO-d6) |
| I-3 | H | AW | 995.8 | 14.76-14.67 (m, 1H), 8.98 (s, 1H), 8.56 (t, J = 5.6 Hz, 1H), 8.30 |
| | | | | (d, J = 5.2 Hz, 1H), 7.93 (d, J = 8.0 Hz, 1H), 7.65 (d, J = 9.2 Hz, |
| | | | | 1H), 7.45-7.35 (m, 5H), 7.30 (s, 1H), 7.21 (t, J = 7.6 Hz, 1H), 6.91- |
| | | | | 6.82 (m, 2H), 6.62 (d, J = 52 Hz, 1H), 5.13 (d, J = 3.2 Hz, 1H), |
| | | | | 4.76 (d, J = 12.4 Hz, 2H), 4.51 (d, J = 9.2 Hz, 1H), 4.46-4.38 (m, |
| | | | | 2H), 4.35 (s, 1H), 4.26-4.14 (m, 2H), 3.70-3.56 (m, 3H), 3.27- |
| | | | | 3.23 (m, 1H), 3.14 (t, J = 8.4 Hz, 2H), 3.01-2.92 (m, 1H), 2.88- |
| | | | | 2.78 (m, 2H), 2.78-2.72 (m, 1H), 2.44 (s, 3H), 2.33 (d, J = 1.6 Hz, |
| | | | | 1H), 2.23-2.07 (m, 4H), 2.07-1.94 (m, 3H), 1.94-1.83 (m, 2H), |
| | | | | 1.82-1.59 (m, 9H), 0.92 (s, 9H) |
| I-4 | H | AW | 995.8 | 14.72 (s, 1H), 8.98 (s, 1H), 8.56 (t, J = 6.0 Hz, 1H), 8.30 (d, J = 5.2 |
| | | | | Hz, 1H), 7.93 (d, J = 8.4 Hz, 1H), 7.66 (d, J = 9.2 Hz, 1H), 7.45- |
| | | | | 7.35 (m, 5H), 7.30 (s, 1H), 7.21 (t, J = 7.2 Hz, 1H), 6.90-6.82 (m, |
| | | | | 2H), 6.62 (d, J = 4.8 Hz, 1H), 5.13 (d, J = 2.4 Hz, 1H), 4.76 (d, J = |
| | | | | 13.2 Hz, 2H), 4.51 (d, J = 9.2 Hz, 1H), 4.47-4.38 (m, 2H), 4.35 (s, |
| | | | | 1H), 4.26-4.14 (m, 2H), 3.67-3.60 (m, 2H), 3.28-3.23 (m, 2H), |
| | | | | 3.18-3.10 (m, 2H), 3.02-2.91 (m, 1H), 2.88-2.79 (m, 2H), 2.75 |
| | | | | (d, J = 11.2 Hz, 1H), 2.67 (d, J = 1.6 Hz, 1H), 2.44 (s, 3H), 2.33 (s, |
| | | | | 1H), 2.17 (t, J = 8.6 Hz, 2H), 2.08-1.95 (m, 5H), 1.94-1.86 (m, |
| | | | | 1H), 1.81-1.72 (m, 4H), 1.72-1.60 (m, 4H), 0.95-0.85 (m, 9H) |
| I-5 | H | AS | 995.9 | 14.72 (s, 1H), 8.98 (s, 1H), 8.56 (t, J = 6.0 Hz, 1H), 8.30 (d, J = 5.2 |
| | | | | Hz, 1H), 7.93 (d, J = 7.6 Hz, 1H), 7.66 (d, J = 9.2 Hz, 1H), 7.44- |
| | | | | 7.36 (m, 5H), 7.30 (s, 1H), 7.25-7.18 (m, 1H), 6.90-6.82 (m, 2H), |
| | | | | 6.62 (d, J = 5.2 Hz, 1H), 5.13 (d, J = 3.6 Hz, 1H), 4.76 (d, J = 12.0 |
| | | | | Hz, 2H), 4.51 (d, J = 9.6 Hz, 1H), 4.42 (t, J = 8.0 Hz, 2H), 4.35 (s, |
| | | | | 1H), 4.25-4.14 (m, 2H), 3.69-3.59 (m, 3H), 3.29 (s, 1H), 3.27- |
| | | | | 3.23 (m, 1H), 3.18-3.09 (m, 2H), 3.02-2.92 (m, 1H), 2.88-2.78 |
| | | | | (m, 2H), 2.78-2.72 (m, 1H), 2.53 (d, J = 6.9 Hz, 2H), 2.47-2.42 |
| | | | | (m, 4H), 2.20-2.11 (m, 2H), 2.07-1.95 (m, 5H), 1.94-1.86 (m, |
| | | | | 1H), 1.82-1.71 (m, 4H), 1.70-1.60 (m, 3H), 0.97-0.86 (m, 9H) |
| I-7 | D | J | 510.3^{b} | 12.44 (s, 1H), 8.99 (d, J = 1.6 Hz, 1H), 8.72 (s, 1H), 8.35 (s, 2H), |
| | | | | 8.23-8.19 (m, 1H), 7.48-7.26 (m, 6H), 6.99-6.94 (m, 2H), 6.13- |
| | | | | 6.03 (m, 2H), 5.15-5.02 (m, 2H), 4.47-4.40 (m, 1H), 4.39-4.22 |
| | | | | (m, 4H), 3.64-3.54 (m, 3H), 3.52-3.41 (m, 4H), 3.00-2.90 (m, |
| | | | | 5H), 2.77-2.60 (m, 4H), 2.46 (d, J = 2.4 Hz, 4H), 2.36-2.31 (m, |
| | | | | 3H), 1.99-1.87 (m, 4H), 1.73-1.67 (m, 4H), 1.55 (d, J = 6.4 Hz, |
| | | | | 4H), 1.26-1.12 (m, 2H), 0.99-0.92 (m, 3H), 0.85-0.78 (m, 3H) |
| I-8 | D | P | 510.3^{b} | 12.44 (s, 1H), 8.99 (d, J = 1.6 Hz, 1H), 8.72 (s, 1H), 8.35 (s, 2H), |
| | | | | 8.23-8.19 (m, 1H), 7.48-7.26 (m, 6H), 6.99-6.94 (m, 2H), 6.13- |
| | | | | 6.03 (m, 2H), 5.15-5.02 (m, 2H), 4.47-4.40 (m, 1H), 4.39-4.22 |
| | | | | (m, 4H), 3.64-3.54 (m, 3H), 3.52-3.41 (m, 4H), 3.00-2.90 (m, |
| | | | | 5H), 2.77-2.60 (m, 4H), 2.46 (d, J = 2.4 Hz, 4H), 2.36-2.31 (m, |
| | | | | 3H), 1.99-1.87 (m, 4H), 1.73-1.67 (m, 4H), 1.55 (d, J = 6.4 Hz, |
| | | | | 4H), 1.26-1.12 (m, 2H), 0.99-0.92 (m, 3H), 0.85-0.78 (m, 3H) |
| I-9 | D | S | 1031.4 | 14.41-14.25 (m, 1H), 12.55-12.37 (m, 1H), 9.02-8.97 (m, 1H), |
| | | | | 8.74-8.70 (m, 1H), 8.55-8.38 (m, 1H), 8.36-8.31 (m, 2H), 8.25- |
| | | | | 8.19 (m, 1H), 7.46-7.28 (m, 5H), 7.00-6.93 (m, 2H), 6.08-6.00 |
| | | | | (m, 1H), 5.82 (d, J = 7.6 Hz, 1H), 5.14-5.00 (m, 2H), 4.48-4.28 |
| | | | | (m, 1H), 3.80-3.67 (m, 1H), 3.58 (s, 2H), 3.52-3.49 (m, 1H), 3.49- |
| | | | | 3.40 (m, 3H), 2.96-2.85 (m, 4H), 2.46 (s, 3H), 2.44 (d, J = 3.2 |
| | | | | Hz, 1H), 2.17 (s, 1H), 2.09-2.03 (m, 1H), 2.01-1.82 (m, 4H), 1.73- |
| | | | | 1.66 (m, 3H), 1.62 (dd, J = 2.8, 11.9 Hz, 2H), 1.56-1.52 (m, 3H), |
| | | | | 1.50-1.38 (m, 2H), 1.34-1.20 (m, 2H), 0.95 (dd, J = 6.8, 8.6 Hz, |
| | | | | 3H), 0.81 (dd, J = 6.8, 14.7 Hz, 3H), 0.70-0.54 (m, 1H) |
| I-10 | D | AC | 502.3^{b} | 14.33 (s, 1H), 12.44 (s, 1H), 9.00 (d, J = 4.0 Hz, 1H), 8.72 (s, 1H), |
| | | | | 8.40-8.55 (m, 1H), 8.34 (s, 2H), 8.21 (d, J = 8.00 Hz, 1H), 7.27- |
| | | | | 7.46 (m, 5H), 6.93-7.01 (m, 2H), 6.04 (q, J = 6.4 Hz, 1H), 5.82 |
| | | | | (d, J = 2.8 Hz, 1H), 5.12 (t, J = 3.6 Hz, 1H), 5.06 (dd, J = 13.6, 5.2 |
| | | | | Hz, 1H), 4.27-4.47 (m, 4H), 3.88 (s, 1H), 3.73-3.80 (m, 2H), 3.55- |
| | | | | 3.68 (m, 2H), 3.37-3.46 (m, 2H), 3.30 (s, 4H), 2.80-2.97 (m, |
| | | | | 4H), 2.46 (d, J = 4.8 Hz, 3H), 2.27-2.37 (m, 2H), 2.17-2.26 (m, |
| | | | | 2H), 1.86-2.01 (m, 3H), 1.71 (d, J = 12.0 Hz, 3H), 1.57-1.65 (m, |
| | | | | 2H), 1.54 (d, J = 6.8 Hz, 3H), 0.90-1.00 (m, 3H), 0.76-0.86 (m, |
| | | | | 3H), 0.54-0.69 (m, 1H) |
| I-11 | D | AE | 502.3^{b} | 14.33 (s, 1H), 12.44 (s, 1H), 9.00 (d, J = 4.0 Hz, 1H), 8.72 (s, 1H), |
| | | | | 8.40-8.55 (m, 1H), 8.34 (s, 2H), 8.21 (d, J = 8.00 Hz, 1H), 7.27- |
| | | | | 7.46 (m, 5H), 6.93-7.01 (m, 2H), 6.04 (q, J = 6.4 Hz, 1H), 5.82 |
| | | | | (d, J = 2.8 Hz, 1H), 5.12 (t, J = 3.6 Hz, 1H), 5.06 (dd, J = 13.6, 5.2 |
| | | | | Hz, 1H), 4.27-4.47 (m, 4H), 3.88 (s, 1H), 3.73-3.80 (m, 2H), 3.55- |
| | | | | 3.68 (m, 2H), 3.37-3.46 (m, 2H), 3.30 (s, 4H), 2.80-2.97 (m, |
| | | | | 4H), 2.46 (d, J = 4.8 Hz, 3H), 2.27-2.37 (m, 2H), 2.17-2.26 (m, |
| | | | | 2H), 1.86-2.01 (m, 3H), 1.71 (d, J = 12.0 Hz, 3H), 1.57-1.65 (m, |
| | | | | 2H), 1.54 (d, J = 6.8 Hz, 3H), 0.90-1.00 (m, 3H), 0.76-0.86 (m, |
| | | | | 3H), 0.54-0.69 (m, 1H) |
| I-12 | D | V | 1031.4 | 14.42-14.27 (m, 1H), 12.52-12.36 (m, 1H), 9.02-8.97 (m, 1H), |
| | | | | 8.75-8.69 (m, 1H), 8.55 (s, 1H), 8.38-8.31 (m, 2H), 8.23-8.18 |
| | | | | (m, 1H), 7.46-7.30 (m, 5H), 7.00-6.93 (m, 2H), 6.12 (d, J = 9.6 |
| | | | | Hz, 1H), 6.08-6.01 (m, 1H), 5.15-5.11 (m, 1H), 5.10-5.01 (m, |
| | | | | 1H), 5.14-5.01 (m, 1H), 4.48-4.27 (m, 4H), 3.58 (dd, J = 2.8, 6.6 |
| | | | | Hz, 1H), 3.45-3.37 (m, 2H), 3.17-3.14 (m, 1H), 3.10-3.03 (m, |
| | | | | 2H), 3.00-2.85 (m, 6H), 2.52 (s, 1H), 2.46-2.45 (m, 3H), 2.43 (d, |
| | | | | J = 5.2 Hz, 1H), 2.30-2.19 (m, 2H), 2.11-1.97 (m, 3H), 1.95- |
| | | | | 1.86 (m, 2H), 1.78-1.70 (m, 3H), 1.68-1.58 (m, 4H), 1.55 (d, J = |
| | | | | 6.4 Hz, 5H), 1.45-1.37 (m, 1H), 1.28-1.22 (m, 1H), 0.95 (dd, J = |
| | | | | 6.4, 9.9 Hz, 3H), 0.86-0.76 (m, 3H) |
| I-13 | D | W | 1031.4 | 14.32 (s, 1H), 12.45 (s, 1H), 9.02-8.98 (m, 1H), 8.71 (s, 1H), 8.54- |
| | | | | 8.41 (m, 1H), 8.36 (s, 2H), 8.21 (d, J = 7.2 Hz, 1H), 7.44-7.30 |
| | | | | (m, 5H), 6.99-6.94 (m, 2H), 6.12 (d, J = 9.2 Hz, 1H), 6.05 (d, J = |
| | | | | 6.8 Hz, 1H), 5.14-5.02 (m, 2H), 4.48-4.29 (m, 4H), 3.61-3.54 |
| | | | | (m, 1H), 3.43 (d, J = 10.0 Hz, 2H), 3.33 (s, 29H), 3.16 (s, 1H), 3.07 |
| | | | | (s, 2H), 3.02-2.89 (m, 6H), 2.55 (s, 1H), 2.46-2.45 (m, 3H), 2.44 |
| | | | | (d, J = 4.8 Hz, 1H), 2.28-2.17 (m, 2H), 2.06-1.97 (m, 3H), 1.96- |
| | | | | 1.88 (m, 2H), 1.75 (s, 3H), 1.61 (s, 4H), 1.55 (d, J = 6.8 Hz, 5H), |
| | | | | 1.46-1.41 (m, 1H), 1.24 (s, 1H), 0.95 (dd, J = 6.4, 9.8 Hz, 3H), |
| | | | | 0.81 (dd, J = 6.8, 14.3 Hz, 3H) |
| I-14 | D | T | 1031.4 | 14.34-14.29 (m, 1H), 12.45 (s, 1H), 9.00 (d, J = 2.0 Hz, 1H), 8.72 |
| | | | | (s, 1H), 8.54-8.39 (m, 1H), 8.34 (s, 2H), 8.21 (dd, J = 1.2, 8.4 Hz, |
| | | | | 1H), 7.50-7.26 (m, 5H), 7.00-6.93 (m, 2H), 6.09-6.01 (m, 1H), |
| | | | | 5.85-5.77 (m, 1H), 5.17-4.98 (m, 2H), 4.47-4.26 (m, 4H), 3.76 |
| | | | | (s, 1H), 3.62-3.57 (m, 2H), 3.52 (s, 1H), 3.47-3.40 (m, 3H), 3.17- |
| | | | | 3.09 (m, 1H), 3.04-2.85 (m, 4H), 2.46 (s, 3H), 2.31-2.09 (m, |
| | | | | 3H), 2.06-1.88 (m, 4H), 1.82-1.73 (m, 3H), 1.70-1.61 (m, 2H), |
| | | | | 1.55 (d, J = 6.8 Hz, 3H), 1.49-1.38 (m, 2H), 1.36-1.20 (m, 3H), |
| | | | | 1.06 (t, J = 6.8 Hz, 1H), 0.99-0.91 (m, 3H), 0.86-0.76 (m, 3H), |
| | | | | 0.70-0.58 (m, 1H) |
| I-15 | D | AN | 1020.9 | 12.44 (s, 1H) 8.99 (s, 1H) 8.72 (s, 1H) 8.43-8.57 (m, 1H) 8.33 (s, |
| | | | | 2H) 8.21 (d, J = 8.0 Hz, 1H) 7.37-7.45 (m, 3H) 7.27-7.34 (m, |
| | | | | 2H) 6.97 (d, J = 8.0 Hz, 2H) 6.03-6.09 (m, 2H) 5.01-5.14 (m, |
| | | | | 2H) 4.38-4.48 (m, 1H) 4.32-4.37 (m, 2H) 4.28 (m, 1H) 3.96 (d, |
| | | | | J = 4.0 Hz, 1H) 3.89 (d, J = 4.0 Hz, 1H) 3.76 (d, J = 12.0 Hz, 1H) |
| | | | | 3.58-3.69 (m, 1H) 3.39-3.49 (m, 2H) 2.92 (s, 3H) 2.42-2.47 (m, |
| | | | | 4H) 2.23-2.38 (m, 6H) 2.00-2.09 (m, 1H) 1.88-1.94 (m, 1H) |
| | | | | 1.70-1.86 (m, 7H) 1.58-1.67 (m, 3H) 1.54 (d, J = 4.0 Hz, 3H) |
| | | | | 1.22-1.31 (m, 2H) 1.06 (m, 1H) 0.96 (t, J = 8.0 Hz, 3H) 0.82 (dd, |
| | | | | J = 46.0 Hz, 8.0 Hz, 3H) |
| I-16 | D | AN | 511.2^{b} | 12.44 (s, 1H) 8.97-8.99 (m, 1H) 8.72 (s, 1H) 8.45-8.58 (m, 1H) |
| | | | | 8.33 (d, J = 4.0 Hz, 2H) 8.22 (d, J = 8.0 Hz, 1H) 7.39-7.44 (m, |
| | | | | 2H) 7.27-7.37 (m, 2H) 6.93-6.99 (m, 2H) 6.00-6.12 (m, 2H) |
| | | | | 4.97-5.19 (m, 2H) 4.22-4.47 (m, 4H) 4.01-4.12 (dd, J = 24.0 |
| | | | | Hz, 8.0 Hz, 2H) 3.77 (d, J = 8.0 Hz, 1H) 3.58-3.66 (m, 1 H) 3.37- |
| | | | | 3.48 (m, 2H) 2.90-3.06 (m, 4H) 2.45 (s, 2H) 2.44 (d, J = 4.0 Hz, |
| | | | | 1H) 2.23-2.37 (m, 4H) 1.98-2.14 (m, 4H) 1.88-1.94 (m, 1H) |
| | | | | 1.72 (m, 2H) 1.58-1.61 (m, 5H) 1.42-1.55 (m, 8H) 1.24 (s, 1H) 0.96 |
| | | | | (t, J = 8.0 Hz, 3H) 0.82 (dd, J = 12.0 Hz, 8.0 Hz, 3H) |
| I-17 | EQ | EP | 943.5 | (CD₃SOCD₃, 298K) δ (ppm) = 8.34 (s, 2H), 7.94 (br d, J = 7.9 Hz, |
| | | | | 1H), 7.43-7.18 (m, 7H), 6.92-6.78 (m, 2H), 5.83-5.76 (m, 1H), |
| | | | | 5.11 (t, J = 3.6 Hz, 1H), 2.08-1.85 (m, 5H), 1.81-1.67 (m, 4H), |
| | | | | 1.64-1.56 (m, 2H), 0.98-0.88 (m, 3H), 0.84-0.74 (m, 3H) |
| I-18^{c} | H | EB | 996.6 | 8.98 (s, 1H), 8.56 (t, J = 6.0 Hz, 1H), 8.23 (s, 2H), 7.94 (d, J = 8.0 |
| | | | | Hz, 1H), 7.67 (br d, J = 9.6 Hz, 1H), 7.43-7.36 (m, 5H), 7.31 (s, |
| | | | | 1H), 7.24-7.18 (m, 1H), 6.89-6.83 (m, 2H), 5.17-5.10 (m, 1H), |
| | | | | 4.62 (br d, J = 12. Hz, 2H), 4.51 (d, J = 9.2 Hz, 1H), 4.46-4.38 (m, |
| | | | | 2H), 4.35 (br s, 1H), 4.25-4.16 (m, 2H), 3.67-3.58 (m, 3H), 3.18- |
| | | | | 3.12 (m, 2H), 3.06 (br s, 1H), 2.99 (br d, J = 4.8 Hz, 5H), 2.96- |
| | | | | 2.91 (m, 1H), 2.73-2.65 (m, 2H), 2.44 (s, 3H), 2.37-2.31 (m, 5H), |
| | | | | 2.21-2.14 (m, 2H), 2.05-1.97 (m, 5H), 1.89 (ddd, J = 4.8, 8.4, |
| | | | | 12.8 Hz, 1H), 1.83-1.76 (m, 1H), 1.72-1.65 (m, 1H), 0.91 (s, 9H) |
| I-19^{c} | H | EB | 996.6 | 8.98 (s, 1H), 8.55 (t, J = 6.0 Hz, 1H), 8.23 (s, 2H), 7.94 (d, J = 8.0 |
| | | | | Hz, 1H), 7.66 (br d, J = 9.6 Hz, 1H), 7.43-7.36 (m, 5H), 7.31 (s, |
| | | | | 1H), 7.23-7.18 (m, 1H), 6.89-6.83 (m, 2H), 5.12 (d, J = 3.6 Hz, |
| | | | | 1H), 4.62 (br d, J = 12.4 Hz, 2H), 4.51 (d, J = 9.2 Hz, 1H), 4.46- |
| | | | | 4.38 (m, 2H), 4.35 (br s, 1H), 4.25-4.16 (m, 2H), 3.67-3.58 (m, |
| | | | | 3H), 3.44 (br dd, J = 4.4, 6.8 Hz, 1H), 3.25-3.22 (m, 1H), 3.17- |
| | | | | 3.11 (m, 1H), 2.99 (br d, J = 4.6 Hz, 5H), 2.69-2.65 (m, 1H), 2.61- |
| | | | | 2.55 (m, 1H), 2.44 (s, 3H), 2.37-2.31 (m, 5H), 2.22-2.06 (m, |
| | | | | 5H), 2.04-1.96 (m, 2H), 1.93-1.85 (m, 2H), 1.83-1.77 (m, 1H), |
| | | | | 1.70 (br dd, J = 8.8, 10.8 Hz, 1H), 0.94-0.89 (m, 9H) |
| I-20 | EO | EP | 935.4 | 8.38-8.30 (m, 2H), 7.94 (d, J = 7.6 Hz, 1H), 7.85-7.72 (m, 2H), |
| | | | | 7.52-7.37 (m, 3H), 7.31 (s, 1H), 7.24-7.18 (m, 1H), 6.89-6.83 |
| | | | | (m, 2H), 5.81 (d, J = 7.6 Hz, 1H), 5.17-5.08 (m, 1H), 4.76-4.66 |
| | | | | (m, 2H), 4.45-4.38 (m, 1H), 4.37-4.32 (m, 3H), 4.24-4.16 (m, |
| | | | | 1H), 3.87 (s, 1H), 3.78-3.71 (m, 2H), 3.71-3.66 (m, 1H), 3.66- |
| | | | | 3.61 (m, 1H), 3.61-3.55 (m, 2H), 3.48-3.42 (m, 2H), 3.16 (d, J = |
| | | | | 2.4 Hz, 1H), 3.13 (d, J = 4.0 Hz, 1H), 3.10 (d, J = 2.4 Hz, 1H), 3.02- |
| | | | | 2.91 (m, 2H), 2.89-2.81 (m, 2H), 2.75 (dd, J = 10.4, 12.8 Hz, |
| | | | | 1H), 2.02-1.91 (m, 3H), 1.80-1.66 (m, 5H), 1.64-1.56 (m, 2H), |
| | | | | 1.25-1.13 (m, 1H), 1.05 (t, J = 7.2 Hz, 2H), 0.98-0.76 (m, 6H), |
| | | | | 0.69-0.55 (m, 1H) |
| I-21 | ET | EM | 1020.3 | 8.38 (d, J = 8.0 Hz, 1H) 8.35 (s, 2H) 7.95 (d, J = 8.0 Hz, 1H) 7.64 |
| | | | | (br d, J = 8.0 Hz, 1H) 7.44-7.50 (m, 3H) 7.40 (br d, J = 8.0 Hz, |
| | | | | 3H) 7.32 (s, 1H) 7.19-7.25 (m, 1H) 6.85-6.89 (m, 2H) 6.37 (d, J = |
| | | | | 4.0 Hz, 1H)5.11 (d, J = 4.0 Hz, 1H) 4.94 (t, J = 8.0 Hz, 1H)4.71- |
| | | | | 4.74 (m, 2H) 4.40-4.51 (m, 2H) 4.28 (br s, 1H) 4.20 (br d, J = 12 |
| | | | | Hz, 1H) 3.85 (s, 3H) 3.55-3.63 (m, 3H) 3.31 (s, 3H) 3.23-3.28 (m, |
| | | | | 1H) 3.11-3.16 (m, 1H) 2.93-3.01 (m, 1H) 2.89-2.91 (m, 2H)2.72- |
| | | | | 2.80 (m, 1H) 2.57-2.64 (m, 1H) 2.35-2.41 (m, 1H) 2.25-2.32 |
| | | | | (m, 1H) 1.96-2.05 (m, 2H) 1.8-1.830 (m, 2H) 1.67-1.76 (m, 5H) |
| | | | | 1.58-1.66 (m, 2H) 1.48-1.57 (m, 4H) 1.43-1.47 (m, 1H) 1.39 (d, |
| | | | | J = 4.0 Hz, 3H) 1.24-1.32 (m, 3H) 0.94 (s, 9 H) |
| I-22 | ET | EM | 1020.4 | 8.38 (d, J = 8.0 Hz, 1H) 8.35 (s, 2H) 7.95 (d, J = 8.0 Hz, 1H) 7.64 |
| | | | | (br d, J = 8.0 Hz, 1H) 7.44-7.50 (m, 3H) 7.40 (br d, J = 8.0 Hz, |
| | | | | 3H) 7.32 (s, 1H) 7.19-7.25 (m, 1H) 6.85-6.90 (m, 2H) 6.37 (d, J = |
| | | | | 4.0 Hz, 1H)5.11 (d, J = 4.0 Hz, 1H) 4.94 (t, J = 8.0 Hz, 1H)4.71- |
| | | | | 4.74 (m, 2H) 4.47-4.49 (m, 1H) 4.41-4.45 (m, 1H) 4.28 (br s, 1H) |
| | | | | 4.20 (br d, J = 12 Hz, 1H) 3.85 (s, 3H) 3.55-3.66 (m, 3H) 3.31 (s, |
| | | | | 3H) 3.23-3.26 (m, 1H) 3.11-3.16 (m, 1H) 2.93-2.99 (m, 1H)2.87- |
| | | | | 2.91 (m, 2H) 2.72-2.80 (m, 1H) 2.57-2.64 (m, 1H) 2.35-2.41 |
| | | | | (m, 1H) 2.26-2.33 (m, 1H) 1.98-2.05 (m, 2H) 1.77-1.83 (m, 2H) |
| | | | | 1.67-1.76 (m, 5H) 1.58-1.66 (m, 2H) 1.48-1.57 (m, 4H) 1.43- |
| | | | | 1.47 (m, 1H) 1.39 (d, J = 4.0 Hz, 3H) 1.24-1.30 (m, 3H) 0.94 (s, 9 H) |
| I-23 | EW | FA | 903.5 | 8.74 (s, 2H), 8.56 (t, J = 6.0 Hz, 1H), 7.98-7.95 (m, 1H), 7.67 (d, |
| | | | | J = 9.6 Hz, 1H), 7.58 (d, J = 8.4 Hz, 2H), 7.46-7.38 (m, 2H), 7.35 |
| | | | | (d, J = 10.4 Hz, 4H), 7.33-7.27 (m, 3H), 7.25-7.19 (m, 1H), 6.91- |
| | | | | 6.83 (m, 2H), 4.85-4.78 (m, 2H), 4.60 (d, J = 9.2 Hz, 1H), 4.45- |
| | | | | 4.35 (m, 3H), 4.32-4.14 (m, 3H), 4.13 (s, 1H), 3.69-3.63 (m, |
| | | | | 3H), 3.25-3.19 (m, 2H), 3.05-2.98 (m, 1H), 2.90-2.82 (m, 1H), |
| | | | | 2.71-2.62 (m, 2H), 2.55 (s, 1H), 2.11-1.95 (m, 3H), 1.94-1.85 |
| | | | | (m, 3H), 1.83-1.76 (m, 1H), 1.70-1.59 (m, 3H), 1.57-1.48 (m, |
| | | | | 1H), 0.96 (s, 9H) |
| I-24 | EW | FA | 903.5 | 8.78-8.73 (m, 2H), 8.59-8.53 (m, 1H), 7.99-7.92 (m, 1H), 7.81- |
| | | | | 7.73 (m, 1H), 7.61-7.54 (m, 2H), 7.39 (s, 2H), 7.38-7.29 (m, |
| | | | | 6H), 7.25-7.19 (m, 1H), 6.92-6.82 (m, 2H), 4.86-4.75 (m, 2H), |
| | | | | 4.58-4.51 (m, 1H), 4.46-4.33 (m, 3H), 4.14 (s, 3H), 3.69-3.62 |
| | | | | (m, 3H), 3.32-3.28 (m, 2H), 3.26 (s, 3H), 3.06-2.97 (m, 1H), 2.90- |
| | | | | 2.82 (m, 1H), 2.56-2.55 (m, 1H), 2.08-1.99 (m, 1H), 1.94-1.73 |
| | | | | (m, 5H), 1.69-1.34 (m, 5H), 1.02-0.90 (m, 9H) |
| I-25 | EW | EM | 950.6 | 8.57-8.50 (m, 1H), 8.38-8.34 (m, 2H), 7.98-7.92 (m, 1H), 7.73- |
| | | | | 7.66 (m, 1H), 7.40 (d, J = 2.4 Hz, 1H), 7.36 (s, 1H), 7.33 (d, J = |
| | | | | 6.8 Hz, 3H), 7.25-7.19 (m, 1H), 6.90-6.83 (m, 2H), 5.15-5.11 |
| | | | | (m, 1H), 4.78-4.66 (m, 2H), 4.50 (d, J = 9.2 Hz, 1H), 4.44-4.37 |
| | | | | (m, 2H), 4.36-4.32 (m, 1H), 4.23-4.15 (m, 2H), 4.13 (s, 1H), 3.70 |
| | | | | (s, 3H), 3.29-3.23 (m, 3H), 3.15-3.10 (m, 1H), 3.01-2.95 (m, |
| | | | | 1H), 2.94-2.84 (m, 2H), 2.81-2.70 (m, 1H), 2.64-2.58 (m, 1H), |
| | | | | 2.05-1.95 (m, 3H), 1.92-1.84 (m, 1H), 1.83-1.77 (m, 1H), 1.76- |
| | | | | 1.65 (m, 6H), 1.62 (d, J = 4.0 Hz, 1H), 1.61-1.58 (m, 1H), 1.56 |
| | | | | (d, J = 2.4 Hz, 1H), 1.54-1.47 (m, 3H), 1.46-1.38 (m, 2H), 1.35- |
| | | | | 1.20 (m, 4H), 0.93 (s, 9H) |
| I-26 | EW | EM | 950.6 | 8.51 (d, J = 2.8 Hz, 1H), 8.37-8.31 (m, 2H), 7.99-7.93 (m, 1H), |
| | | | | 7.74-7.65 (m, 1H), 7.42-7.38 (m, 1H), 7.36 (s, 1H), 7.33 (d, J = |
| | | | | 6.4 Hz, 3H), 7.25-7.19 (m, 1H), 6.91-6.82 (m, 2H), 5.15-5.09 |
| | | | | (m, 1H), 4.77-4.67 (m, 2H), 4.55-4.48 (m, 1H), 4.44-4.37 (m, |
| | | | | 2H), 4.33 (br d, J = 5.6 Hz, 1H), 4.25-4.17 (m, 2H), 4.17-4.13 |
| | | | | (m, 1H), 3.68-3.58 (m, 3H), 3.29-3.22 (m, 3H), 3.17-3.10 (m, |
| | | | | 1H), 3.01-2.95 (m, 1H), 2.94-2.85 (m, 2H), 2.80-2.72 (m, 1H), |
| | | | | 2.66-2.58 (m, 1H), 2.06-1.94 (m, 3H), 1.91-1.86 (m, 1H), 1.85- |
| | | | | 1.80 (m, 1H), 1.79-1.67 (m, 5H), 1.67-1.64 (m, 1H), 1.63 (s, |
| | | | | 1H), 1.60 (s, 1H), 1.56-1.46 (m, 3H), 1.41 (d, J = 13.8 Hz, 2H), |
| | | | | 1.36-1.23 (m, 4H), 0.93 (s, 9H) |
| I-27 | H | AW | 995.8 | 14.77-14.71 (m, 1H), 9.00 (s, 1H), 8.58 (t, J = 6.2 Hz, 1H), 8.32 |
| | | | | (d, J = 5.2 Hz, 1H), 7.95 (d, J = 7.6 Hz, 1H), 7.67 (d, J = 9.2 Hz, |
| | | | | 1H), 7.47-7.38 (m, 5H), 7.32 (s, 1H), 7.26-7.20 (m, 1H), 6.91- |
| | | | | 6.85 (m, 2H), 6.64 (d, J = 5.2 Hz, 1H), 5.15 (d, J = 3.6 Hz, 1H), |
| | | | | 4.82-4.75 (m, 2H), 4.53 (d, J = 9.2 Hz, 1H), 4.49-4.41 (m, 2H), |
| | | | | 4.37 (s, 1H), 4.28-4.15 (m, 2H), 3.71-3.61 (m, 3H), 3.31 (s, 1H), |
| | | | | 3.29-3.24 (m, 1H), 3.22-3.10 (m, 2H), 2.98 (dt, J = 2.4 Hz, J = |
| | | | | 12.0 Hz, 1H), 2.90-2.80 (m, 2H), 2.77 (d, J = 10.8 Hz, 1H), 2.56 |
| | | | | (s, 1H), 2.46 (s, 3H), 2.24-2.15 (m, 2H), 2.15-2.10 (m, 2H), 2.09 |
| | | | | (d, J = 5.6 Hz, 1H), 2.06-1.98 (m, 2H), 1.97-1.86 (m, 2H), 1.84- |
| | | | | 1.76 (m, 4H), 1.74 (d, J = 5.6 Hz, 1H), 1.73-1.62 (m, 3H), 0.94 |
| | | | | (s, 9H) |
| I-28 | H | G | 489.9^{b} | 9.33 (s, 1H) 8.99 (s, 1H) 8.57 (t, J = 8.0 Hz, 1H) 8.37 (s, 1H) 8.13- |
| | | | | 8.15 (dd, J = 8.0 Hz, 4.0 Hz, 1H) 7.66 (d, J = 12.0 Hz, 1H) 7.58- |
| | | | | 7.62 (dd, J = 8.0 Hz, 4.0 Hz, 1H) 7.53 (d, J = 12.0 Hz, 1H) 7.37- |
| | | | | 7.44 (m, 4H) 7.31-7.37 (m, 1H) 7.00-7.06 (m, 2H) 5.14 (s, 1H) |
| | | | | 4.52 (d, J = 8.0 Hz, 1H) 4.40-4.47 (m, 2H) 4.36 (br , s, 1H) 4.19- |
| | | | | 4.25 (dd, J = 12.0 Hz, 8.0 Hz, 1H) 3.69 (br s, 2H) 3.11-3.18 (m, |
| | | | | 1H) 2.96-3.02 (m, 2H) 2.78 (t, J = 12.0 Hz, 2H) 2.60-2.67 (m, |
| | | | | 1H) 2.47 (s, 1H) 2.45 (m, 3H) 2.11-2.33 (m, 6H) 1.95-2.06 (m, |
| | | | | 5H) 1.84-1.93 (m, 3H) 1.60-1.78 (m, 9H) 1.40-1.45 (m, 2H) |
| | | | | 0.92 (s, 9H) |
| I-29 | H | G | 489.8^{b} | 9.33 (s, 1H) 8.99 (s, 1H) 8.57 (t, J = 8.0 Hz, 1H) 8.37 (s, 1H) 8.13- |
| | | | | 8.15 (dd, J = 8.0 Hz, 4.0 Hz, 1H) 7.57-7.69 (m, 2H) 7.53 (d, J = |
| | | | | 8.0 Hz, 1H) 7.37-7.46 (m, 4H) 7.32-7.36 (m, 1H) 7.00-7.06 (m, |
| | | | | 2H) 5.14 (d, J = 4.0 Hz, 1H) 4.52 (d, J = 8.0 Hz, 1H) 4.38-4.48 |
| | | | | (m, 2H) 4.36 (br s, 1H) 4.18-4.26 (m, 1H) 3.66 (br s, 2H) 3.14 (m, |
| | | | | 1H) 2.99 (d, J = 8.0 Hz, 2H) 2.78 (t, J = 8.0 Hz, 2H) 2.62-2.64 (m, |
| | | | | 1H) 2.46 (s, 1H) 2.45 (s, 3H) 2.21-2.31 (m, 3H) 1.98-2.19 (m, |
| | | | | 6H) 1.84-1.97 (m, 5H) 1.67-1.80 (m, 6H) 1.59-1.67 (m, 3H) |
| | | | | 1.39-1.42 (m, 2H) 0.92 (s, 9H) |

^{a}The coupling was ran anywhere from 25-40° C. for 4-16 hr. The absolute stereochemistry of the final compounds was assigned arbitrarily for those that had separate diastereomers.
^{b}LCMS data reported as (M/2 + H)⁺.
^{c}The coupled product was further separated by SFC: column: DAICEL CHIRALPAK IA (250 mm × 30 mm, 10 um); mobile phase: [0.1% NH3H2O IPA]; B %: 75%-75%, 10; 100 min.

TABLE 3A
Compounds synthesized via Method 1 using the corresponding amines and acids for the coupling.

| | | | LCMS (ESI+) | |
|---|---|---|---|---|
| I-#^{a} | Amine | Acid | m/z (M + H)⁺ | ¹H NMR (400 MHz, DMSO-d6) |
| I-74^{b} | GP | GN | 940.4 | 8.45 (t, J = 8.0 Hz, 1H) 8.35-8.33 (m, 2H) 7.95 (d, J = 8.00 Hz, |
| | | | | 1H) 7.39 (d, J = 4.0 Hz, 1H) 7.36-7.29 (m, 4H) 7.24-7.20 (m, |
| | | | | 1H) 7.05 (d, J = 8.0 Hz, 1H) 6.93-6.89 (m, 1H) 6.87-6.84 (m, |
| | | | | 2H) 6.06 (s, 1H) 5.11 (m, 1H) 4.76-4.70 (m, 2H) 4.32 (br t, J = |
| | | | | 8.0 Hz, 2H) 4.25-4.13 (m, 1H) 4.24-4.11 (m, 2H) 4.10-4.04 (m, |
| | | | | 3H) 3.87-3.85 (m, 3H) 3.78-3.71 (m, 1H) 3.67-3.60 (m, 2H) |
| | | | | 3.44 (br d, J = 8.0 Hz, 1H) 3.27-3.22 (m, 2H) 3.17-3.10 (m, 1H) |
| | | | | 2.98 (br d, J = 9.88 Hz, 3H) 2.79-2.73 (m, 1H) 2.40-2.36 (m, 3H) |
| | | | | 2.01-1.93 (m, 3H) 1.78-1.69 (m, 5H) 1.68-1.60 (m, 5H) 0.97- |
| | | | | 0.94 (m, 3H) 0.81-0.77 (m, 3 H) |
| I-75^{c} | H | HL | 1000.5 | 8.99 (s, 1H), 8.60-8.51 (m, 1H), 8.22 (s, 1H), 7.92 (d, J = 7.6 Hz, |
| | | | | 1H), 7.89-7.88 (m, 1H), 7.66 (d, J = 7.6 Hz, 1H), 7.44-7.32 (m, |
| | | | | 5H), 7.25-7.17 (m, 2H), 6.89-6.78 (m, 2H), 4.51 (d, J = 9.2 Hz, |
| | | | | 1H), 4.44-4.39 (m, 2H), 4.38-4.31 (m, 1H), 4.26-4.16 (m, 2H), |
| | | | | 4.08-3.98 (m, 1H), 3.21-3.14 (m, 4H), 3.04 (s, 3H), 2.92 (dt, J = |
| | | | | 1.6, 3.0 Hz, 2H), 2.68 (s, 1H), 2.15 (d, J = 7.2 Hz, 4H), 1.77 (s, |
| | | | | 3H), 1.67 (d, J = 0.8 Hz, 4H), 1.39 (d, J = 7.2 Hz, 4H), 0.92 (s, 9H) |
| I-76^{d} | H | GR | 993.7 | 12.43 (br s, 1H) 8.99 (s, 1H) 8.70 (s, 1H) 8.56 (t, J = 8.0 Hz, 1H) |
| | | | | 8.24-8.22 (m, 2H) 8.20 (d, J = 4.0 Hz, 1H) 7.67 (d, J = 8.0 Hz, |
| | | | | 1H) 7.46-7.41 (m, 2H) 7.40-7.37 (m, 2H) 7.32-7.28 (m, 1H) 6.98- |
| | | | | 6.95 (m, 2H) 6.00-5.95 (m, 1H) 5.14-5.12 (m, 1H) 4.98-4.93 (m, |
| | | | | 1H) 4.52 (d, J = 8.0 Hz, 1H) 4.47-4.43 (m, 1H) 4.42-4.40 (m, |
| | | | | 1H) 4.36 (br s, 1H) 4.25-4.20 (m, 1H) 3.66 (br s, 2H) 3.20-3.11 |
| | | | | (m, 2H) 2.99-2.97 (m, 4H) 2.94-2.85 (m, 2H) 2.60-2.55 (m, 1H) |
| | | | | 2.45 (s, 3H) 2.35 (br s, 4H) 2.21-2.15 (m, 2H) 2.07-2.05 (m, 3H) |
| | | | | 2.02-1.98 (m, 3H) 1.94-1.87 (m, 1H) 1.82-1.78 (m, 1H) 1.71-1.66 |
| | | | | (m, 1H) 1.53 (d, J = 4.0 Hz, 3H) 0.92 (s, 9 H) |
| I-77^{d} | H | GR | 993.7 | 12.44 (s, 1H) 8.99 (s, 1H) 8.70 (s, 1H) 8.56 (t, J = 8.0 Hz, 1H) 8.24- |
| | | | | 8.20 (m, 2H) 8.20 (d, J = 4.0 Hz, 1H) 7.66 (d, J = 8.0 Hz, 1H) |
| | | | | 7.47-7.40 (m, 2H) 7.40-7.37 (m, 2H) 7.32-7.28 (m, 1H) 6.98-6.94 |
| | | | | (m, 2H) 6.00-5.95 (m, 1H) 5.16-5.10 (m, 1H) 4.98-4.93 (m, 1H) |
| | | | | 4.52 (d, J = 8.0 Hz, 1H) 4.47-4.43 (m, 1H) 4.42-4.40 (m, 1H) |
| | | | | 4.36 (br s, 1H) 4.24-4.19 (m, 1H) 3.66 (br s, 2H) 3.20-3.11 (m, |
| | | | | 2H) 2.99-2.97 (m, 4H) 2.94-2.85 (m, 2H) 2.60-2.55 (m, 1H) 2.45 |
| | | | | (s, 3H) 2.35 (br s, 4H) 2.21-2.16 (m, 2H) 2.02-2.07 (m, 3H) 2.02- |
| | | | | 1.98 (m, 3H) 1.94-1.87 (m, 1H) 1.82-1.78 (m, 1H) 1.71-1.66 (m, |
| | | | | 1H) 1.53 (d, J = 4.0 Hz, 3H) 0.92 (s, 9H). |
| I-78 | H | GS | 1021.5 | 14.05-14.50 (m, 1 H), 12.43 (s, 1 H), 8.99 (s, 1 H), 8.70 (s, 1 H), |
| | | | | 8.56 (t, J = 6.00 Hz, 1 H), 8.19-8.26 (m, 3 H), 7.65 (d, J = 9.20 Hz, |
| | | | | 1 H), 7.36-7.46 (m, 4 H), 7.27-7.33 (m, 1 H), 6.93-7.00 (m, 2 |
| | | | | H), 5.87-6.06 (m, 1 H), 5.07-5.22 (m, 1 H), 4.96 (dd, J = 13.60, |
| | | | | 5.20 Hz, 1 H), 4.50-4.57 (m, 1 H), 4.32-4.48 (m, 3 H), 4.22 (dd, |
| | | | | J = 16.0, 5.60 Hz, 1 H), 3.63-3.70 (m, 2 H), 3.08-3.24 (m, 2 H), |
| | | | | 2.82-3.03 (m, 6 H), 2.60 (s, 4 H), 2.45 (s, 3 H), 2.11-2.22 (m, 1 |
| | | | | H), 2.00-2.08 (m, 1 H), 1.85-1.99 (m, 3 H), 1.56-1.84 (m, 6 H), |
| | | | | 1.53 (d, J = 6.40 Hz, 3 H), 1.07-1.35 (m, 4 H), 0.90-0.97 (m, 9 H) |
| I-79 | H | GS | 1021.5 | 13.83-14.66 (m, 1 H), 12.13-12.76 (m, 1 H), 8.99 (s, 1 H), 8.70 |
| | | | | (s, 1 H), 8.56 (t, J = 6.00 Hz, 1 H), 8.13-8.29 (m, 3 H), 7.64 (d, |
| | | | | J = 9.20 Hz, 1 H), 7.35-7.50 (m, 4 H), 7.23-7.33 (m, 1 H), 6.89- |
| | | | | 7.04 (m, 2 H), 5.85-6.05 (m, 1 H), 5.05-5.24 (m, 1 H), 4.96 (dd, |
| | | | | J = 13.20, 5.20 Hz, 1 H), 4.53 (d, J = 9.20 Hz, 1 H), 4.31-4.48 (m, 3 |
| | | | | H), 4.22 (dd, J = 15.60, 5.60 Hz, 1 H), 3.62-3.74 (m, 2 H), 3.09- |
| | | | | 3.24 (m, 2 H), 2.86-3.03 (m, 6 H), 2.55-2.65 (m, 4 H), 2.41-2.47 |
| | | | | (m, 3 H), 2.10-2.25 (m, 1 H), 1.99-2.08 (m, 1 H), 1.73-1.96 (m, |
| | | | | 6H), 1.57-1.72 (m, 3 H), 1.52 (d, J = 6.80 Hz, 3 H), 1.08-1.34 (m, |
| | | | | 4H), 0.86-0.97 (m, 9 H) |
| I-80^{d, e} | H | GM | 511.5 | 14.41-14.24 (m, 1H), 12.58-12.23 (m, 1H), 8.98 (s, 1H), 8.70- |
| | | | | 8.63 (m, 1H), 8.60-8.51 (m, 1H), 8.23-8.18 (m, 1H), 7.94-7.88 |
| | | | | (m, 1H), 7.70-7.60 (m, 1H), 7.43-7.37 (m, 4H), 7.30 (t, J = 7.6 |
| | | | | Hz, 1H), 7.00-6.93 (m, 2H), 6.09 (d, J = 5.6 Hz, 1H), 6.03-5.92 |
| | | | | (m, 1H), 5.15-5.11 (m, 1H), 5.10-5.00 (m, 1H), 4.57-4.49 (m, |
| | | | | 1H), 4.47-4.38 (m, 2H), 4.35 (d, J = 4.4 Hz, 1H), 4.27-4.16 (m, |
| | | | | 1H), 3.69-3.62 (m, 2H), 3.55 (s, 3H), 3.30 (s, 2H), 3.20-3.11 (m, |
| | | | | 2H), 2.95-2.82 (m, 2H), 2.44 (s, 3H), 2.07-1.98 (m, 2H), 1.97- |
| | | | | 1.84 (m, 4H), 1.83-1.73 (m, 2H), 1.72-1.62 (m, 4H), 1.51 (d, J = |
| | | | | 6.4 Hz, 3H), 1.31-1.21 (m, 4H), 1.20-1.13 (m, 2H), 0.92 (s, 9H) |
| I-81^{d, e} | H | GM | 511.5 | 14.40-14.25 (m, 1H), 12.50-12.20 (m, 1H), 9.05-8.92 (m, 1H), |
| | | | | 8.71-8.62 (m, 1H), 8.56 (t, J = 6.1 Hz, 1H), 8.30-8.11 (m, 1H), |
| | | | | 7.91 (d, J = 6.0 Hz, 1H), 7.68-7.59 (m, 1H), 7.44-7.36 (m, 4H), |
| | | | | 7.32-7.26 (m, 1H), 7.01-6.89 (m, 2H), 6.13-5.92 (m, 2H), 5.17- |
| | | | | 5.00 (m, 2H), 4.57-4.49 (m, 1H), 4.47-4.39 (m, 2H), 4.35 (d, J = |
| | | | | 1.5 Hz, 1H), 4.21 (m J = 5.2 15.6 Hz, 1H), 3.66 (s, 2H), 3.55 (s, |
| | | | | 3H), 3.19-3.10 (m, 2H), 2.94-2.83 (m, 2H), 2.44 (s, 3H), 2.24- |
| | | | | 2.10 (m, 2H), 2.06-1.96 (m, 2H), 1.94-1.86 (m, 2H), 1.84-1.76 |
| | | | | (m, 4H), 1.65 (s, 2H), 1.61-1.57 (m, 1H), 1.51 (d, J = 6.4 Hz, 3H), |
| | | | | 1.30-1.21 (m, 4H), 1.21-1.09 (m, 3H), 0.95-0.89 (m, 9H) |
| I-83^{d} | FF | HB | 914.3 | 8.99 (s, 1H), 8.42-8.36 (m, 1H), 8.02-7.86 (m, 2H), 7.46-7.41 |
| | | | | (m, 2H), 7.38 (br d, J = 7.6 Hz, 3H), 7.26 (s, 1H), 7.25-7.17 (m, |
| | | | | 3H), 7.15-7.09 (m, 2H), 6.86 (d, J = 7.6 Hz, 2H), 5.10 (d, J = 3.2 |
| | | | | Hz, 1H), 4.97-4.87 (m, 1H), 4.51-4.47 (m, 1H), 4.43 (t, J = 8.4 |
| | | | | Hz, 1H), 4.32-4.22 (m, 1H), 3.62-3.54 (m, 3H), 3.45-3.41 (m, |
| | | | | 1H), 3.39-3.38 (m, 1H), 2.83 (br dd, J = 2.5, 8.6 Hz, 1H), 2.61- |
| | | | | 2.56 (m, 2H), 2.46 (br s, 7H), 2.35-2.31 (m, 3H), 2.06-1.94 (m, |
| | | | | 2H), 1.86-1.72 (m, 4H), 1.41-1.34 (m, 3H), 1.28-1.20 (m, 1H), |
| | | | | 0.95-0.86 (m, 9H) |
| I-85 | H | FA | 976.4 | 8.95-9.01 (m, 1 H), 8.74 (s, 2 H), 8.53-8.64 (m, 1 H), 7.96 (d, |
| | | | | J = 8.00 Hz, 1 H), 7.67 (d, J = 9.20 Hz, 1 H), 7.58 (d, J = 8.00 Hz, 2 |
| | | | | H), 7.38-7.44 (m, 5 H), 7.28-7.35 (m, 3 H), 7.20-7.25 (m, 1 H), |
| | | | | 6.80-6.92 (m, 2 H), 5.08-5.21 (m, 1 H), 4.81 (dd, J = 9.20, 3.20 |
| | | | | Hz, 2 H), 4.60 (d, J = 9.20 Hz, 1 H), 4.33-4.49 (m, 3 H), 4.18-4.28 |
| | | | | (m, 2 H), 3.60-3.74 (m, 3 H), 3.20-3.25 (m, 1 H), 2.93-3.12 (m, |
| | | | | 2 H), 2.83-2.90 (m, 1 H), 2.62-2.70 (m, 2 H), 2.45 (s, 3 H), 2.01- |
| | | | | 2.09 (m, 2 H), 1.75-1.99 (m, 5 H), 1.43-1.73 (m, 5 H), 0.97 (s, 9 H) |
| I-86 | H | FA | 976.4 | 8.99 (s, 1 H), 8.75 (s, 2 H), 8.57 (t, J = 6.00 Hz, 1 H), 7.92-8.00 (m, |
| | | | | 1 H), 7.78 (d, J = 9.60 Hz, 1 H), 7.58 (d, J = 8.20 Hz, 2 H), 7.37-7.46 |
| | | | | (m, 5 H), 7.28-7.36 (m, 3 H), 7.18-7.26 (m, 1 H), 6.80-6.92 (m, |
| | | | | 2 H), 5.15 (d, J = 3.20 Hz, 1 H), 4.76-4.86 (m, 2 H), 4.55 (d, J = 9.20 |
| | | | | Hz, 1 H), 4.40-4.49 (m, 2 H), 4.37 (s, 1 H), 4.19-4.29 (m, 2 H), |
| | | | | 3.60-3.75 (m, 3 H), 3.36-3.49 (m, 2 H), 3.19-3.27 (m, 2 H), 3.02 |
| | | | | (td, J = 12.00, 2.80 Hz, 1 H), 2.80-2.91 (m, 1 H), 2.56-2.71 (m, 1 |
| | | | | H), 2.46 (s, 3 H), 2.30-2.38 (m, 1 H), 2.00-2.10 (m, 1 H), 1.75- |
| | | | | 1.95 (m, 5 H), 1.41-1.59 (m, 4 H), 0.96 (s, 9 H) |
| I-97^{d} | GX | EM | 968.3 | 14.72 (s, 1H), 8.57 (t, J = 6.4 Hz, 1H), 8.36 (s, 2H), 7.95 (d, J = |
| | | | | 15.2 Hz, 1H), 7.70 (d, J = 9.6 Hz, 1H), 7.50 (t, J = 7.6 Hz, 1H), |
| | | | | 7.40 (br d, J = 2.8 Hz, 1H), 7.28-7.34 (m, 2H), 7.16-7.25 (m, |
| | | | | 2H), 6.87 (t, J = 8.0 Hz, 2H), 5.13 (d, J = 3.6 Hz, 1H), 4.66-4.79 |
| | | | | (m, 2H), 4.50 (d, J = 9.2 Hz, 1H), 4.32-4.44 (m, 3H), 4.27 (s, 1H), |
| | | | | 4.16-4.26 (m, 2H), 3.60-3.66 (m, 2H), 3.31 (br s, 3H), 3.08-3.16 |
| | | | | (m, 1H), 2.86-3.00 (m, 3H), 2.72-2.80 (m, 1H), 1.94-2.04 (m, |
| | | | | 2H), 1.77-1.92 (m, 3H), 1.67-1.76 (m, 6H), 1.47-1.64 (m, 1H), |
| | | | | 1.39-1.46 (m, 2H), 1.24-1.34 (m, 3H), 0.91 (s, 9H) |
| I-98^{d} | HN | EM | 984.7 | 8.65 (t, J = 4.8 Hz, 1H) 8.35 (s, 2H) 7.95 (d, J = 8.0 Hz, 1H) 7.71 |
| | | | | (d, J = 9.2 Hz, 1H)7.58 (d, J = 8.0 Hz, 1H) 7.55 (d, J = 1.2 Hz, 1H) |
| | | | | 7.40 (br d, J = 2.8 Hz, 1H) 7.33-7.29 (m, 2H) 7.24-7.19 (m, 1H) |
| | | | | 6.89-6.84 (m, 2H) 4.96-5.33 (m, 1H) 4.74-4.72 (m, 2H) 4.50 (d, |
| | | | | J = 8.0 Hz, 1H) 4.43-4.40 (m, 1H) 4.39-4.32 (m, 2H) 4.30 (s, 1H) |
| | | | | 4.25-4.18 (m, 2H) 3.65-3.60 (m, 3H) 3.28-3.23 (m, 2H) 3.19- |
| | | | | 3.09 (m, 2H) 2.99-2.87 (m, 3H) 2.76-2.73 (m, 1H) 2.64-2.58 |
| | | | | (m, 1H) 2.53 (d, J = 1.6 Hz, 1H) 2.40-2.35 (m, 1H) 2.34-2.29 (m, |
| | | | | 1H) 2.06-1.96 (m, 2H) 1.93-1.86 (m, 1H) 1.82-1.77 (m, 1H) |
| | | | | 1.72 (br d, J = 12.0 Hz, 4H) 1.66-1.59 (m, 3H) 1.54-1.49 (m, 2H) |
| | | | | 1.46-1.38 (m, 3H) 1.33-1.24 (m, 3H) 0.92 (s, 9H). |
| I-99^{d} | HN | EM | 985.0 | 8.65 (t, J = 4.8 Hz, 1H) 8.35 (s, 2H) 7.95 (d, J = 8.0 Hz, 1H) 7.71 |
| | | | | (d, J = 9.2 Hz, 1H)7.58 (d, J = 8.0 Hz, 1H) 7.55 (d, J = 1.2 Hz, 1H) |
| | | | | 7.40 (br d, J = 2.8 Hz, 1H) 7.33-7.30 (m, 2H) 7.24-7.20 (m, 1H) |
| | | | | 6.89-6.84 (m, 2H) 4.96-5.34 (m, 1H) 4.74-4.72 (m, 2H) 4.50 (d, |
| | | | | J = 8.0 Hz, 1H) 4.43-4.40 (m, 1H) 4.39-4.32 (m, 2H) 4.30 (s, 1H) |
| | | | | 4.25-4.18 (m, 2H) 3.65-3.60 (m, 3H) 3.28-3.22 (m, 2H) 3.19- |
| | | | | 3.09 (m, 2H) 2.99-2.87 (m, 3H) 2.76-2.73 (m, 1H) 2.64-2.58 |
| | | | | (m, 1H) 2.53 (d, J = 1.6 Hz, 1H) 2.40-2.34 (m, 1H) 2.34-2.29 (m, |
| | | | | 1H) 2.06-1.96 (m, 2H) 1.93-1.86 (m, 1H) 1.82-1.77 (m, 1H) |
| | | | | 1.72 (br d, J = 12.0 Hz, 4H) 1.66-1.59 (m, 3H) 1.54-1.49 (m, 2H) |
| | | | | 1.46-1.38 (m, 3H) 1.33-1.24 (m, 3H) 0.91 (s, 9H) |
| I-100^{d} | HP | EM | 980.5 | 14.78-14.62 (m, 1H), 8.45-8.33 (m, 3H), 7.94 (br d, J = 7.2 Hz, |
| | | | | 1H), 7.78-7.67 (m, 1H), 7.44-7.30 (m, 3H), 7.22 (br t, J = 6.8 |
| | | | | Hz, 1H), 7.03 (s, 1H), 6.94 (br d, J = 7.2 Hz, 1H), 6.90-6.84 (m, |
| | | | | 2H), 5.16-5.08 (m, 1H), 4.77-4.66 (m, 2H), 4.54-4.48 (m, 1H), |
| | | | | 4.47.4.40 (ₘ, 1H), 4.38-4.31 (m, 1H), 4.31-4.18 (m, 2H), 4.17- |
| | | | | 4.07 (m, 2H), 3.82 (s, 3H), 3.69-3.55 (m, 4H), 3.49-3.40 (m, |
| | | | | 1H), 3.38-3.35 (m, 1H), 3.27-3.23 (m, 1H), 3.22-3.11 (m, 2H), |
| | | | | 3.04-2.88 (m, 2H), 2.83-2.74 (m, 2H), 2.21-2.12 (m, 1H), 2.05- |
| | | | | 1.97 (m, 3H), 1.96-1.78 (m, 5H), 1.65-1.58 (m, 2H), 1.56-1.51 |
| | | | | (m, 1H), 1.65 (br s, 1H), 1.47-1.22 (m, 7H), 0.92 (br s, 9H) |
| I-101^{d} | GU | EM | 984.6 | 14.92-14.53 (m, 1H), 8.44 (t, J = 5.6 Hz, 1H), 8.35 (s, 2H), 7.95 |
| | | | | (d, J = 8.0 Hz, 1H), 7.66-7.59 (m, 1H), 7.48-7.39 (m, 5H), 7.32 |
| | | | | (s, 1H), 7.25-7.18 (m, 1H), 6.92-6.82 (m, 2H), 5.12 (d, J = 3.6 |
| | | | | Hz, 1H), 4.76-4.67 (m, 2H), 4.48 (d, J = 9.2 Hz, 1H), 4.38-4.29 |
| | | | | (m, 2H), 4.20 (br d, J = 12 Hz, 1H), 4.12-4.09 (m, 1H), 3.68-3.53 |
| | | | | (m, 4H), 3.27-3.22 (m, 2H), 3.20-3.08 (m, 2H), 3.00-2.83 (m, |
| | | | | 4H), 2.80-2.71 (m, 2H), 2.68 (td, J = 1.6, 3.6 Hz, 2H), 2.30-2.23 |
| | | | | (m, 1H), 1.95 (br s, 2H), 1.74-1.66 (m, 5H), 1.39 (br s, 7H), 1.33- |
| | | | | 1.23 (m, 3H), 0.95-0.88 (m, 9H) |
| I-103^{d} | HE | EM | 964.6 | 8.56-8.47 (m, 1H), 8.35 (s, 2H), 7.98-7.93 (m, 1H), 7.72-7.66 |
| | | | | (m, 1H), 7.40 (d, J = 2.4 Hz, 1H), 7.32 (s, 1H), 7.27 (s, 3H), 7.24- |
| | | | | 7.19 (m, 1H), 6.90-6.83 (m, 2H), 5.12 (d, J = 3.2 Hz, 1H), 4.77- |
| | | | | 4.65 (m, 2H), 4.50 (d, J = 9.6 Hz, 1H), 4.42-4.31 (m, 3H), 4.24- |
| | | | | 4.12 (m, 2H), 3.71-3.56 (m, 3H), 3.27-3.22 (m, 2H), 3.19-3.08 |
| | | | | (m, 2H), 3.02-2.95 (m, 1H), 2.93-2.85 (m, 2H), 2.81-2.71 (m, |
| | | | | 1H), 2.60 (t, J = 7.6 Hz, 1H), 2.41-2.37 (m, 1H), 2.31-2.25 (m, |
| | | | | 1H), 2.03 (s, 3H), 1.99 (d, J = 1.2 Hz, 1H), 1.87 (ddd, J = 4.4, 8.8, |
| | | | | 12.8 Hz, 1H), 1.83-1.77 (m, 1H), 1.75-1.65 (m, 6H), 1.64-1.54 |
| | | | | (m, 3H), 1.54-1.47 (m, 3H), 1.46-1.36 (m, 2H), 1.33-1.21 (m, |
| | | | | 4H), 0.93 (s, 9H) |
| I-104^{d} | HF | EM | 998.3 | 14.81-14.57 (m, 1H), 8.44 (t, J = 5.2 Hz, 1H), 8.34 (s, 2H), 7.97- |
| | | | | 7.91 (m, 1H), 7.61 (d, J = 9.2 Hz, 1H), 7.41-7.37 (m, 2H), 7.37- |
| | | | | 7.30 (m, 2H), 7.26-7.19 (m, 2H), 6.90-6.82 (m, 2H), 5.11 (d, J = |
| | | | | 3.6 Hz, 1H), 4.75-4.68 (m, 2H), 4.47 (d, J = 9.2 Hz, 1H), 4.38- |
| | | | | 4.29 (m, 2H), 4.23-4.17 (m, 1H), 4.16-4.05 (m, 2H), 3.69-3.56 |
| | | | | (m, 3H), 3.28-3.23 (m, 2H), 3.13 (dd, J = 10.4, 12.4 Hz, 1H), 3.03- |
| | | | | 2.95 (m, 1H), 2.93-2.85 (m, 2H), 2.75 (dd, J = 10.8, 12.8 Hz, |
| | | | | 1H), 2.64-2.56 (m, 1H), 2.35 (s, 3H), 2.30-2.24 (m, 1H), 2.04- |
| | | | | 1.95 (m, 2H), 1.88-1.78 (m, 2H), 1.72-1.57 (m, 7H), 1.54-1.37 |
| | | | | (m, 7H), 1.30-1.21 (m, 3H), 0.92 (s, 9H) |
| I-105^{d} | HH | IK | 984.5 | 8.55 (t, J = 6.0 Hz, 1H), 8.36 (s, 2H), 7.97 (dd, J = 1.2, 8.2 Hz, 1H), |
| | | | | 7.70 (d, J = 9.2 Hz, 1H), 7.56 (d, J = 2.4 Hz, 1H), 7.40-7.27 (m, |
| | | | | 6H), 6.85 (t, J = 8.0 Hz, 1H), 5.13 (d, J = 3.2 Hz, 1H), 4.79-4.64 |
| | | | | (m, 2H), 4.50 (d, J = 9.2 Hz, 1H), 4.45-4.31 (m, 3H), 4.26-4.09 |
| | | | | (m, 3H), 3.73-3.57 (m, 3H), 3.30-3.21 (m, 2H), 3.19-3.09 (m, |
| | | | | 1H), 3.05-2.82 (m, 3H), 2.81-2.71 (m, 1H), 2.53 (s, 2H), 2.43- |
| | | | | 2.36 (m, 1H), 2.35-2.28 (m, 1H), 2.06-1.95 (m, 2H), 1.92-1.77 |
| | | | | (m, 2H), 1.75-1.58 (m, 7H), 1.57-1.46 (m, 4H), 1.46-1.38 (m, |
| | | | | 2H), 1.33-1.23 (m, 3H), 0.93 (s, 9H) |
| I-106^{d} | GY | EM | 982.5 | 8.43 (t, J = 4.4 Hz, 1H), 8.34 (d, J = 1.3 Hz, 2H), 7.94 (d, J = 8.3 |
| | | | | Hz, 1H), 7.61 (d, J = 8.3 Hz, 1H), 7.44-7.35 (m, 2H), 7.31 (s, 1H), |
| | | | | 7.21 (t, J = 7.3 Hz, 1H), 7.15 (d, J = 9.9 Hz, 1H), 7.06-6.97 (m, |
| | | | | 1H), 6.91-6.81 (m, 2H), 5.11 (s, 1H), 4.75-4.68 (m, 2H), 4.49- |
| | | | | 4.44 (m, 1H), 4.35 (s, 1H), 4.32 (d, J = 7.3 Hz, 1H), 4.19 (d, J = |
| | | | | 12.4 Hz, 1H), 4.11 (d, J = 3.1 Hz, 1H), 3.59 (s, 3H), 3.12 (s, 1H), |
| | | | | 2.95 (s, 2H), 2.87 (s, 1H), 2.75 (s, 2H), 2.66 (s, 2H), 2.36 (s, 3H), |
| | | | | 2.30-2.21 (m, 2H), 2.03-1.92 (m, 3H), 1.84-1.77 (m, 2H), 1.73- |
| | | | | 1.64 (m, 6H), 1.55-1.45 (m, 5H), 1.44-1.37 (m, 2H), 1.31-1.21 |
| | | | | (m, 4H), 0.95-0.87 (m, 9H) |
| I-112^{d} | H | GZ | 511.5^{e} | 14.34 (s, 1H), 12.43 (s, 1H), 8.99 (s, 1H), 8.70 (s, 1H), 8.56 (t, J = |
| | | | | 6.0 Hz, 1H), 8.26-8.19 (m, 3H), 7.71 (d, J = 9.2 Hz, 1H), 7.44- |
| | | | | 7.37 (m, 4H), 7.33-7.27 (m, 1H), 6.99-6.93 (m, 2H), 6.02-5.94 |
| | | | | (m, 1H), 5.13 (d, J = 3.6 Hz, 1H), 5.00-4.93 (m, 1H), 4.51 (d, J = |
| | | | | 9.6 Hz, 1H), 4.47-4.40 (m, 2H), 4.35 (s, 1H), 4.26-4.18 (m, 1H), |
| | | | | 3.70-3.59 (m, 2H), 3.01 (s, 4H), 2.97-2.87 (m, 2H), 2.70-2.61 |
| | | | | (m, 1H), 2.55-2.53 (m, 1H), 2.45 (s, 3H), 2.42-2.24 (m, 6H), 2.09- |
| | | | | 1.96 (m, 2H), 1.94-1.86 (m, 1H), 1.84-1.77 (m, 1H), 1.72 (d, J = |
| | | | | 12 Hz, 1H), 1.60-1.47 (m, 8H), 1.46-1.39 (m, 2H), 1.36-1.05 |
| | | | | (m, 4H), 0.93 (s, 8H) |
| I-113^{d} | HH | HL | 927.5 | 8.54 (t, J = 6.0 Hz, 1H), 7.94-7.88 (m, 1H), 7.64 (d, J = 9.2 Hz, |
| | | | | 1H), 7.45-7.39 (m, 1H), 7.37-7.29 (m, 5H), 7.22-7.17 (m, 2H), |
| | | | | 6.87-6.80 (m, 2H), 5.12 (d, J = 3.6 Hz, 1H), 5.21-4.93 (m, 1H), |
| | | | | 4.50 (d, J = 9.2 Hz, 1H), 4.43-4.32 (m, 3H), 4.20-4.10 (m, 2H), |
| | | | | 4.06-4.00 (m, 1H), 3.69-3.60 (m, 2H), 3.46 (d, J = 4.4 Hz, 1H), |
| | | | | 3.16 (d, J = 9.2 Hz, 2H), 3.13-3.08 (m, 1H), 3.04 (d, J = 8.4 Hz, |
| | | | | 2H), 2.88 (d, J = 10.0 Hz, 2H), 2.83 (s, 1H), 2.80-2.72 (m, 2H), |
| | | | | 2.52 (s, 2H), 2.30-2.19 (m, 2H), 2.17-2.04 (m, 6H), 2.02-1.86 |
| | | | | (m, 7H), 1.78-1.71 (m, 3H), 1.66-1.59 (m, 4H), 1.40-1.29 (m, |
| | | | | 4H), 0.90 (s, 9H) |
| I-114^{d} | GU | EM | 984.6 | 14.86-14.54 (m, 1H), 8.44 (t, J = 5.4 Hz, 1H), 8.35 (s, 2H), 7.98- |
| | | | | 7.92 (m, 1H), 7.63 (br d, J = 9.1 Hz, 1H), 7.48-7.38 (m, 5H), 7.32 |
| | | | | (s, 1H), 7.25-7.19 (m, 1H), 6.91-6.83 (m, 2H), 5.11 (d, J = 3.5 |
| | | | | Hz, 1H), 4.76-4.68 (m, 2H), 4.47 (d, J = 9.3 Hz, 1H), 4.38-4.28 |
| | | | | (m, 2H), 4.20 (br d, J = 12.5 Hz, 1H), 4.10 (t, J = 5.4 Hz, 1H), 3.69- |
| | | | | 3.55 (m, 3H), 3.27 (br d, J = 11.1 Hz, 2H), 3.17-3.09 (m, 1H), |
| | | | | 3.00-2.85 (m, 3H), 2.80-2.72 (m, 1H), 2.63-2.54 (m, 3H), 2.44- |
| | | | | 2.37 (m, 1H), 2.27 (br t, J = 10.9 Hz, 1H), 2.04-1.92 (m, 2H), |
| | | | | 1.90-1.77 (m, 2H), 1.70 (br d, J = 9.0 Hz, 5H), 1.60 (br d, J = 12.8 |
| | | | | Hz, 2H), 1.53-1.32 (m, 6H), 1.30-1.23 (m, 2H), 0.97-0.88 (m, 9H) |
| I-115^{d} | GY | EM | 982.6 | 8.43 (t, J = 5.4 Hz, 1H), 8.34 (s, 2H), 7.97-7.91 (m, 1H), 7.61 (d, |
| | | | | J = 9.5 Hz, 1H), 7.41-7.36 (m, 2H), 7.31 (s, 1H), 7.24-7.18 (m, |
| | | | | 1H), 7.15 (dd, J = 2.9, 10.0 Hz, 1H), 7.02 (dt, J = 2.4, 8.5 Hz, 1H), |
| | | | | 6.90-6.82 (m, 2H), 5.10 (d, J = 3.5 Hz, 1H), 4.75-4.68 (m, 2H), |
| | | | | 4.46 (d, J = 9.4 Hz, 1H), 4.38-4.32 (m, 1H), 4.32-4.29 (m, 1H), |
| | | | | 4.22-4.17 (m, 1H), 4.11 (d, J = 4.5 Hz, 1H), 3.66-3.55 (m, 3H), |
| | | | | 3.23 (s, 1H), 3.17-3.09 (m, 2H), 2.97 (dd, J = 3.0, 12.1 Hz, 1H), |
| | | | | 2.87 (d, J = 8.6 Hz, 2H), 2.79-2.71 (m, 2H), 2.36 (s, 3H), 2.00- |
| | | | | 1.94 (m, 2H), 1.88-1.76 (m, 3H), 1.74-1.64 (m, 6H), 1.63-1.55 |
| | | | | (m, 3H), 1.48 (dd, J = 3.1, 8.1 Hz, 3H), 1.44-1.39 (m, 2H), 1.38 |
| | | | | (s, 1H), 1.31-1.22 (m, 4H), 0.92 (s, 9H) |
| I-116^{d} | GX | EM | 968.5 | 14.73 (s, 1H), 8.57 (br t, J = 5.82 Hz, 1H), 8.35 (s, 2H), 7.95 (d, J = |
| | | | | 7.25 Hz, 1H), 7.70 (d, J = 7.2 Hz, 1H), 7.50 (t, J = 8.0 Hz, 1H), |
| | | | | 7.40 (d, J = 2.8 Hz, 1H), 7.27-7.34 (m, 2H), 7.15-7.25 (m, 2H), |
| | | | | 6.87 (t, J = 8.0 Hz, 2H), 5.13 (d, J = 3.2 Hz, 1H), 4.72-4.77 (m, |
| | | | | 1H), 4.50 (d, J = 9.6 Hz, 1H), 4.37-4.44 (m, 1H), 4.34 (br s, 1H), |
| | | | | 4.28 (s, 1H), 4.17-4.26 (m, 2H), 3.60-3.65 (m, 2H), 3.31 (br s, |
| | | | | 2H), 3.22-3.28 (m, 1H), 3.09-3.18 (m, 1H), 2.95-3.01 (m, 1H), |
| | | | | 2.85-2.93 (m, 2H), 2.72-2.81 (m, 1H), 2.58-2.64 (m, 1H), 2.25- |
| | | | | 2.31 (m, 1H), 1.94-2.07 (m, 2H), 1.77-1.92 (m, 2H), 1.58-1.77 |
| | | | | (m, 8H), 1.34-1.55 (m, 6H), 1.22-1.34 (m, 3H), 0.86-0.96 (m, 9H) |
| I-117^{d} | HE | EM | 964.7 | 8.51 (t, J = 5.6 Hz, 1H), 8.35 (s, 2H), 7.95 (d, J = 8.0 Hz, 1H), 7.69 |
| | | | | (d, J = 9.2 Hz, 1H), 7.40 (d, J = 3.2 Hz, 1H), 7.32 (s, 1H), 7.27 (s, |
| | | | | 3H), 7.24-7.19 (m, 1H), 6.91-6.82 (m, 2H), 5.12 (d, J = 3.2 Hz, |
| | | | | 1H), 4.76-4.67 (m, 2H), 4.50 (d, J = 9.2 Hz, 1H), 4.43-4.31 (m, |
| | | | | 3H), 4.24-4.12 (m, 2H), 3.69-3.57 (m, 3H), 3.27-3.23 (m, 2H), |
| | | | | 3.18-3.09 (m, 2H), 2.98 (dd, J = 3.2, 12.0 Hz, 1H), 2.94-2.85 (m, |
| | | | | 2H), 2.80-2.72 (m, 1H), 2.63-2.57 (m, 1H), 2.40-2.38 (m, 1H), |
| | | | | 2.30 (d, J = 4.0 Hz, 1H), 2.03 (s, 3H), 1.99 (d, J = 1.6 Hz, 1H), 1.88 |
| | | | | (dt, J = 4.4, 8.4 Hz, 1H), 1.84-1.77 (m, 1H), 1.76-1.65 (m, 6H), |
| | | | | 1.61 (d, J = 10.4 Hz, 3H), 1.55-1.46 (m, 3H), 1.43 (d, J = 4.8 Hz, |
| | | | | 2H), 1.36-1.22 (m, 4H), 0.93 (s, 9H) |
| I-118^{d} | H | GZ | 511.5^{e} | 14.43-14.28 (m, 1H), 12.43 (s, 1H), 8.99 (s, 1H), 8.70 (s, 1H), 8.56 |
| | | | | (t, J = 6.0 Hz, 1H), 8.25-8.19 (m, 3H), 7.70 (d, J = 9.2 Hz, 1H), |
| | | | | 7.45-7.37 (m, 4H), 7.33-7.26 (m, 1H), 7.00-6.92 (m, 2H), 6.01- |
| | | | | 5.94 (m, 1H), 5.13 (d, J = 3.6 Hz, 1H), 5.00-4.93 (m, 1H), 4.50 |
| | | | | (d, J = 9.6 Hz, 1H), 4.47-4.40 (m, 2H), 4.35 (s, 1H), 4.27-4.18 |
| | | | | (m, 1H), 3.71-3.58 (m, 2H), 3.43-3.38 (m, 1H), 3.01 (s, 4H), 2.97- |
| | | | | 2.84 (m, 2H), 2.70-2.61 (m, 1H), 2.55 (s, 1H), 2.45 (s, 3H), 2.43- |
| | | | | 2.21 (m, 6H), 2.08-1.96 (m, 2H), 1.94-1.86 (m, 1H), 1.84-1.76 |
| | | | | (m, 1H), 1.72 (d, J = 12.1 Hz, 1H), 1.61 (d, J = 12 Hz, 1H), 1.53 (d, |
| | | | | J = 6.4 Hz, 5H), 1.49-1.33 (m, 4H), 1.33-1.22 (m, 3H), 0.93 (s, 8H) |
| I-125^{d} | HP | EM | 980.8 | 8.42 (br t, J = 6.0 Hz, 1H), 8.35 (s, 2H), 7.94 (br d, J = 8.0 Hz, 1H), |
| | | | | 7.74 (br d, J = 9.6 Hz, 1H), 7.45-7.39 (m, 1H), 7.38-7.28 (m, |
| | | | | 2H), 7.26-7.19 (m, 1H), 7.03 (s, 1H), 6.96-6.92 (m, 1H), 6.90- |
| | | | | 6.84 (m, 2H), 5.13 (d, J = 3.6 Hz, 1H), 4.73 (br d, J = 12.4 Hz, 2H), |
| | | | | 4.51 (d, J = 9.6 Hz, 1H), 4.43 (t, J = 8.0 Hz, 1H), 4.35 (br s, 1H), |
| | | | | 4.29 (br d, J = 6.0 Hz, 1H), 4.26-4.18 (m, 1H), 4.30-4.18 (m, |
| | | | | 1H), 4.17-4.07 (m, 2H), 3.82 (s, 3H), 3.65-3.59 (m, 3H), 3.27 (br |
| | | | | s, 2H), 3.20-3.09 (m, 2H), 3.05-2.91 (m, 3H), 2.85-2.74 (m, |
| | | | | 3H), 2.09-1.96 (m, 5H), 1.89 (ddd, J = 4.8, 8.4, 12.4 Hz, 3H), 1.78- |
| | | | | 1.60 (m, 4H), 1.49 (br d, J = 3.2 Hz, 1H), 1.42-1.21 (m, 7H), |
| | | | | 0.92 (s, 9H) |
| I-126^{d} | HF | EM | 998.3 | 14.72 (s, 1H), 8.44 (t, J = 5.2 Hz, 1H), 8.34 (s, 2H), 7.94 (d, J = 8.0 |
| | | | | Hz, 1H), 7.61 (d, J = 8.8 Hz, 1H), 7.41-7.37 (m, 2H), 7.37-7.31 |
| | | | | (m, 2H), 7.26-7.19 (m, 2H), 6.90-6.83 (m, 2H), 5.10 (d, J = 3.6 |
| | | | | Hz, 1H), 4.75-4.68 (m, 2H), 4.46 (d, J = 9.2 Hz, 1H), 4.38-4.30 |
| | | | | (m, 2H), 4.22-4.17 (m, 1H), 4.15-4.05 (m, 2H), 3.68-3.56 (m, |
| | | | | 3H), 3.28-3.22 (m, 2H), 3.16-3.09 (m, 1H), 3.00-2.94 (m, 1H), |
| | | | | 2.93-2.85 (m, 2H), 2.75 (dd, J = 10.8, 12.8 Hz, 1H), 2.63-2.56 |
| | | | | (m, 1H), 2.35 (s, 3H), 2.30-2.22 (m, 1H), 2.03-1.95 (m, 2H), 1.88- |
| | | | | 1.78 (m, 2H), 1.71-1.58 (m, 7H), 1.53-1.36 (m, 6H), 1.35-1.22 |
| | | | | (m, 4H), 0.92 (s, 9H) |
| I-129^{d} | IT | EM | 526.6^{e} | 8.41 (t, J = 6.0 Hz, 1H), 8.35 (s, 2H), 7.94 (d, J = 8.0 Hz, 1H), 7.72- |
| | | | | 7.65 (m, 1H), 7.41-7.29 (m, 3H), 7.24-7.18 (m, 1H), 7.00 (d, J = |
| | | | | 1.1 Hz, 1H), 6.95-6.90 (m, 1H), 6.89-6.82 (m, 2H), 5.12 (d, J = |
| | | | | 2.5 Hz, 1H), 4.71 (td, J = 4.2, 8.7 Hz, 2H), 4.50 (d, J = 9.6 Hz, |
| | | | | 1H), 4.43 (t, J = 8.0 Hz, 1H), 4.34 (s, 1H), 4.31-4.23 (m, 1H), 4.20 |
| | | | | (d, J = 12.0 Hz, 1H), 4.16-4.09 (m, 2H), 4.03 (t, J = 6.0 Hz, 2H), |
| | | | | 3.68-3.57 (m, 3H), 3.20-3.06 (m, 3H), 2.99-2.85 (m, 3H), 2.80- |
| | | | | 2.70 (m, 1H), 2.65-2.58 (m, 1H), 2.42 (d, J = 6.8 Hz, 2H), 2.39- |
| | | | | 2.35 (m, 1H), 2.30 (s, 1H), 2.19 (s, 6H), 2.05-1.95 (m, 2H), 1.93- |
| | | | | 1.84 (m, 3H), 1.83-1.64 (m, 7H), 1.61 (d, J = 12.0 Hz, 2H), 1.57- |
| | | | | 1.47 (m, 4H), 1.43 (d, J = 10.0 Hz, 2H), 1.32-1.22 (m, 3H), 0.91 |
| | | | | (s, 9H) |
| I-130^{d} | JP | EM | 1038.7 | 14.72 (m, 1H), 8.47-8.39 (m, 1H), 8.35 (s, 2H), 7.95 (d, J = 8.1 |
| | | | | Hz, 1H), 7.71 (d, J = 9.2 Hz, 1H), 7.43-7.30 (m, 3H), 7.26-7.16 |
| | | | | (m, 1H), 7.01 (s, 1H), 6.93 (d, J = 8.4 Hz, 1H), 6.90-6.83 (m, 2H), |
| | | | | 5.14 (d, J = 2.0 Hz, 1H), 4.78-4.65 (m, 2H), 4.54-4.47 (m, 1H), |
| | | | | 4.50 (d, J = 9.6 Hz, 1H), 4.44 (t, J = 8.0 Hz, 1H), 4.35 (s, 1H), 4.32- |
| | | | | 4.24 (m, 1H), 4.20 (d, J = 12.4 Hz, 1H), 4.14 (s, 2H), 4.05 (t, J = |
| | | | | 6.4 Hz, 2H), 3.69-3.57 (m, 3H), 3.50 (t, J = 6.4 Hz, 2H), 3.26 (s, |
| | | | | 4H), 3.18-3.08 (m, 1H), 3.02-2.84 (m, 3H), 2.76 (dd, J = 10.8, |
| | | | | 12.8 Hz, 1H), 2.53 (d, J = 2.0 Hz, 2H), 2.19-2.08 (m, 1H), 2.07- |
| | | | | 1.85 (m, 6H), 1.80 (d, J = 6.0 Hz, 1H), 1.77-1.55 (m, 8H), 1.50 (d, |
| | | | | J = 7.2 Hz, 3H), 1.42 (t, J = 9.6 Hz, 2H), 1.34-1.19 (m, 3H), 0.91 |
| | | | | (s, 9H) |
| I-137^{c} | HH | HL | 927.5 | 8.53 (t, J = 6.0 Hz, 1H), 7.91 (dd, J = 1.2, 8.4 Hz, 1H), 7.62 (d, J = |
| | | | | 9.2 Hz, 1H), 7.37-7.30 (m, 5H), 7.22-7.17 (m, 2H), 6.87-6.81 |
| | | | | (m, 2H), 5.11 (d, J = 3.6 Hz, 1H), 4.50 (d, J = 9.2 Hz, 1H), 4.44- |
| | | | | 4.32 (m, 3H), 4.20-4.11 (m, 2H), 4.03 (d, J = 11, 6 Hz, 1H), 3.67- |
| | | | | 3.60 (m, 2H), 3.49-3.44 (m, 1H), 3.18-3.14 (m, 2H), 3.11 (d, J = |
| | | | | 8.4 Hz, 1H), 3.04 (d, J = 8.4 Hz, 2H), 2.90-2.72 (m, 5H), 2.52 (s, |
| | | | | 2H), 2.21-2.04 (m, 9H), 1.99 (d, J = 9.6 Hz, 1H), 1.95-1.83 (m, |
| | | | | 4H), 1.77-1.70 (m, 3H), 1.67-1.56 (m, 5H), 1.41-1.30 (m, 4H), |
| | | | | 0.91 (s, 9H) |
| I-143^{f} | KD | KC | 980.6 | 14.40-14.26 (m, 1H), 12.47-12.44 (m, 1H), 8.72 (s, 1H), 8.44-8.39 |
| | | | | (m, 1H), 8.34 (s, 2H), 8.27-8.20 (m, 1H), 7.33-7.28 (m, 2H), 7.03 |
| | | | | (s, 1H), 6.98-6.94 (m, 3H), 6.07-6.02 (m, 1H), 5.66-5.64 (d, J = 9.6 |
| | | | | Hz, 1H), 5.10-5.03 (m, 2H), 4.89-4.86 (m, 1H), 4.45-4.41 (m, 1H), |
| | | | | 4.37-4.21 (m, 4H), 4.13 (s, 1H), 4.04-4.02 (m, 2H), 3.90-3.83 (m, |
| | | | | 2H), 3.79-3.72 (m, 4H), 3.64 (m, 2H), 2.97-2.85 (m, 4H), 2.28-2.24 |
| | | | | (m, 2H), 2.08-1.89 (m, 6H), 1.78-1.69 (m, 4H), 1.64-1.54 (m, 5H), |
| | | | | 0.91 (s, 9H) |
| I-145^{g} | KE | KC | 1022.5 | 14.59-13.96 (m, 1H), 12.50-12.34 (m, 1H), 8.77-8.63 (m, 1H), |
| | | | | 8.41 (s, 1H), 8.33 (s, 2H), 8.24-8.17 (m, 1H), 7.39-7.22 (m, 2H), |
| | | | | 7.05-6.86 (m, 4H), 6.09-5.97 (m, 1H), 5.72-5.59 (m, 1H), 5.20- |
| | | | | 4.94 (m, 2H), 4.49-4.39 (m, 1H), 4.36-4.21 (m, 3H), 4.19-4.08 |
| | | | | (m, 2H), 4.06-3.95 (m, 2H), 3.91-3.80 (m, 2H), 3.79-3.69 (m, |
| | | | | 2H), 3.67-3.57 (m, 2H), 3.46-3.40 (m, 2H), 3.02-2.79 (m, 5H), |
| | | | | 2.43-2.36 (m, 3H), 2.24 (s, 2H), 2.06-1.87 (m, 6H), 1.80-1.67 |
| | | | | (m, 4H), 1.64-1.49 (m, 5H), 0.90 (s, 9H) |
| I-149^{d} | JP | EM | 1038.7 | 14.72 (s, 1H), 8.41 (t, J = 6.0 Hz, 1H), 8.35 (s, 2H), 7.97-7.91 (m, |
| | | | | 1H), 7.70 (d, J = 9.2 Hz, 1H), 7.39 (d, J = 3.2 Hz, 1H), 7.37-7.29 |
| | | | | (m, 2H), 7.24-7.18 (m, 1H), 7.00 (d, J = 1.2 Hz, 1H), 6.95-6.90 |
| | | | | (m, 1H), 6.89-6.83 (m, 2H), 5.12 (d, J = 3.6 Hz, 1H), 4.81-4.64 |
| | | | | (m, 2H), 4.49 (d, J = 9.2 Hz, 1H), 4.43 (t, J = 8.0 Hz, 1H), 4.34 (d, |
| | | | | J = 2.0 Hz, 1H), 4.31-4.23 (m, 1H), 4.20 (d, J = 12.4 Hz, 1H), 4.16- |
| | | | | 4.12 (m, 1H), 4.12-4.07 (m, 1H), 4.07-4.00 (m, 2H), 3.68-3.58 |
| | | | | (m, 3H), 3.49 (t, J = 6.4 Hz, 2H), 3.28 (s, 1H), 3.25 (s, 3H), 3.17- |
| | | | | 3.07 (m, 1H), 3.01-2.94 (m, 1H), 2.93-2.83 (m, 2H), 2.75 (dd, J = |
| | | | | 10.8, 12.8 Hz, 1H), 2.58 (d, J = 7.6 Hz, 2H), 2.06-1.93 (m, 5H), |
| | | | | 1.93-1.85 (m, 1H), 1.84-1.66 (m, 6H), 1.66-1.57 (m, 3H), 1.55- |
| | | | | 1.47 (m, 2H), 1.46-1.37 (m, 3H), 1.35-1.22 (m, 3H), 1.17 (t, J = |
| | | | | 7.2 Hz, 1H), 0.91 (s, 9H) |
| I-152^{d} | HH | IK | 984.6 | 8.55 (t, J = 6.0 Hz, 1H), 8.35 (s, 2H), 8.00-7.94 (m, 1H), 7.70 (d, |
| | | | | J = 9.2 Hz, 1H), 7.57 (d, J = 2.8 Hz, 1H), 7.41-7.30 (m, 6H), 6.85 |
| | | | | (t, J = 8.0 Hz, 1H), 5.12 (d, J = 3.2 Hz, 1H), 4.72 (t, J = 10.8 Hz, |
| | | | | 2H), 4.50 (d, J = 9.2 Hz, 1H), 4.45-4.30 (m, 3H), 4.25-4.11 (m, |
| | | | | 3H), 3.68-3.59 (m, 3H), 3.31-3.26 (m, 2H), 3.21-3.08 (m, 1H), |
| | | | | 3.05-2.84 (m, 3H), 2.81-2.72 (m, 1H), 2.59 (d, J = 7.6 Hz, 2H), |
| | | | | 2.38 (s, 1H), 2.31-2.25 (m, 1H), 2.05-1.93 (m, 2H), 1.91-1.78 |
| | | | | (m, 2H), 1.74-1.58 (m, 8H), 1.53-1.37 (m, 5H), 1.32-1.23 (m, |
| | | | | 3H), 0.93 (s, 9H) |
| I-153^{d} | HH | IQ | 984.5 | 8.54 (t, J = 6.0 Hz, 1H) 8.36 (s, 2H) 7.96 (d, J = 8.4 Hz 1H) 7.69 |
| | | | | (d, J = 9.2 Hz 1H) 7.56 (d, J = 2.8 Hz 1H) 7.38-7.32 (m, 6H) 6.87- |
| | | | | 5.83 (m, 1H) 5.12 (br s, 1H) 4.72 (t, J = 9.2, 2.8 Hz, 2H) 4.50 (d, |
| | | | | J = 5.6, 1H) 4.38 (d, J = 6.0, 3H) 4.34-4.20 (m, 3H) 4.19-4.13 |
| | | | | (m, 3H) 3.65-3.62 (m, 3H) 3.14 (s, 2H) 2.89-2.68 (m, 7H) 2.01- |
| | | | | 1.71 (m, 5H) 1.69-1.49 (m, 8H) 1.31-1.28 (m, 6H) 0.92 (s, 9H) |
| I-154^{d} | HH | IQ | 985.0 | 8.54 (t, J = 6.0 Hz, 1H) 8.36 (s, 2H) 7.97 (d, J = 8.0 Hz 1H) 7.69 |
| | | | | (d, J = 9.2 Hz 1H) 7.56 (d, J = 2.8 Hz 1H) 7.38-7.32 (m, 6H) 6.87- |
| | | | | 5.83 (m, 1H) 5.12 (br s, 1H) 4.72 (t, J = 9.2, 2.8 Hz, 2H) 4.50 (d, |
| | | | | J = 5.6, 1H) 4.38 (d, J = 6.0, 3H) 4.34-4.20 (m, 3H) 4.19-4.13 |
| | | | | (m, 3H) 3.65-3.62 (m, 3H) 3.14 (s, 2H) 2.89-2.68 (m, 7H) 2.01- |
| | | | | 1.71 (m, 5H) 1.69-1.49 (m, 8H) 1.43-1.30 (m, 6H) 1.31-1.28 |
| | | | | (m, 3H) 0.93 (s, 9H) |
| I-157^{d} | HH | ID | 968.5 | 8.54 (t, J = 6.19 Hz, 1 H), 8.35 (s, 2 H), 7.80 (d, J = 8.20 Hz, 1 H), |
| | | | | 7.69 (d, J = 9.20 Hz, 1 H), 7.52 (d, J = 3, 20 Hz, 1 H), 7.25-7.41 (m, |
| | | | | 5 H), 7.13-7.21 (m, 1 H), 6.82 (td, J = 8.00, 5.20 Hz, 1 H), 5.12 (d, |
| | | | | J = 2.80 Hz, 1 H), 4.72 (t, J = 10.80 Hz, 2 H), 4.50 (d, J = 9.20 Hz, 1 |
| | | | | H), 4.30-4.45 (m, 3 H), 4.07-4.28 (m, 3 H), 3.53-3.70 (m, 3 H), |
| | | | | 3.23-3.30 (m, 2 H), 3.08-3.20 (m, 1 H), 2.84-3.04 (m, 3 H), 2.76 |
| | | | | (dd, J = 12.80, 10.80 Hz, 1 H), 2.57-2.69 (m, 1 H), 2.23-2.43 (m, |
| | | | | 3 H), 1.93-2.08 (m, 2 H), 1.59-1.90 (m, 10 H), 1.37-1.54 (m, 5 |
| | | | | H), 1.18-1.34 (m, 3 H), 0.93 (s, 8 H) |
| I-158^{d} | IT | EM | 526.5^{e} | 8.41 (t, J = 6.0 Hz, 1H), 8.35 (s, 2H), 7.94 (d, J = 8.0 Hz, 1H), 7.69 |
| | | | | (d, J = 9.6 Hz, 1H), 7.39 (d, J = 3.2 Hz, 1H), 7.34 (d, J = 7.6 Hz, |
| | | | | 1H), 7.31 (s, 1H), 7.24-7.18 (m, 1H), 7.00 (d, J = 1.2 Hz, 1H), 6.92 |
| | | | | (dd, J = 1.1, 8.0 Hz, 1H), 6.89-6.83 (m, 2H), 5.11 (d, J = 3.6 Hz, |
| | | | | 1H), 4.75-4.67 (m, 2H), 4.49 (d, J = 9.6 Hz, 1H), 4.43 (t, J = 8.0 |
| | | | | Hz, 1H), 4.34 (s, 1H), 4.30-4.23 (m, 1H), 4.20 (d, J = 12.4 Hz, |
| | | | | 1H), 4.16-4.08 (m, 2H), 4.03 (t, J = 6.4 Hz, 2H), 3.68-3.57 (m, |
| | | | | 3H), 3.28 (s, 1H), 3.27-3.20 (m, 2H), 3.13 (dt, J = 2.8, 12.4 Hz, |
| | | | | 1H), 3.00-2.94 (m, 1H), 2.93-2.84 (m, 2H), 2.75 (dd, J = 10.8, |
| | | | | 12.8 Hz, 1H), 2.60 (t, J = 7.6 Hz, 1H), 2.37 (t, J = 7.2 Hz, 3H), 2.31- |
| | | | | 2.25 (m, 1H), 2.15 (s, 6H), 2.05-1.94 (m, 2H), 1.93-1.88 (m, |
| | | | | 1H), 1.88-1.83 (m, 2H), 1.79 (d, J = 7.6 Hz, 1H), 1.75-1.66 (m, |
| | | | | 5H), 1.66-1.57 (m, 3H), 1.54-1.47 (m, 2H), 1.45-1.36 (m, 3H), |
| | | | | 1.33-1.22 (m, 3H), 0.91 (s, 9H) |
| I-166 | KP | KC | 936.4 | 0.91 (s, 9 H) 1.54 (d, 7 = 6.4 Hz, 3 H) 1.67-1.82 (m, 4 H) 1.87- |
| | | | | 1.97 (m, 3 H) 1.98-2.05 (m, 1 H) 2.22-2.30 (m, 2 H) 2.32-2.35 |
| | | | | (m, 2 H) 2.84-2.89 (m, 3 H) 2.90-2.98 (m, 3 H) 3.63 (s, 2 H) 3.72 |
| | | | | (d, 7 = 8.0 Hz, 1 H) 3.75-3.79 (m, 1H)3.83 (d, 7 = 8.0 Hz, 1H)3.87- |
| | | | | 3.90 (m, 1 H) 4.04-4.07 (m, 1 H) 4.10-4.15 (m, 1 H) 4.18-4.24 |
| | | | | (m, 1 H) 4.29-4.36 (m, 2 H) 4.42 (t, 7 = 8.00 Hz, 1 H) 5.02-5.09 |
| | | | | (m, 1 H) 5.09-5.13 (m, 1 H) 5.62-5.68 (m, 1 H) 6.01-6.09 (m, 1 |
| | | | | H) 6.78-6.82 (m, 1 H) 6.83-6.87 (m, 1 H) 6.93-7.00 (m, 2 H) |
| | | | | 7.24 (d, 7 = 8 Hz, 1 H) 7.27-7.33 (m, 1 H) 8.18-8.24 (m, 1 H) 8.34 |
| | | | | (s, 2 H) 8.41-8.46 (m, 1 H) 8.66-8.79 (m, 1 H) 9.76-9.84 (m, 1 |
| | | | | H) 12.36-12.48 (m, 1 H) 14.18-14.44 (m, 1 H) |
| I-167 | KQ | KC | 964.4 | 14.32 (s, 1H), 12.56-12.28 (m, 1H), 8.71 (s, 1H), 8.33 (s, 2H), 8.31 |
| | | | | (d, J = 8.0 Hz, 1H), 8.21 (J = 1.3, 8.4 Hz, 1H), 7.33-7.26 (m, 1H), |
| | | | | 7.20 (d, J = 7.6 Hz, 1H), 7.06-7.00 (m, 2H), 6.99-6.92 (m, 2H), |
| | | | | 6.04 (q, J = 6.4 Hz, 1H), 5.55 (d, J = 9.2 Hz, 1H), 5.18-5.01 (m, |
| | | | | 3H), 4.42 (t, J = 8.0 Hz, 1H), 4.32-4.23 (m, 2H), 4.13 (s, 1H), 3.91- |
| | | | | 3.78 (m, 5H), 3.77-3.69 (m, 2H), 3.57 (s, 2H), 3.44-3.37 (m, |
| | | | | 2H), 3.00-2.89 (m, 2H), 2.85 (d, J = 10.0 Hz, 2H), 2.37 (s, 1H), |
| | | | | 2.25 (t, J = 7.2 Hz, 2H), 2.04-1.96 (m, 1H), 1.91 (t, J = 9.6 Hz, |
| | | | | 2H), 1.81-1.66 (m, 5H), 1.65-1.56 (m, 2H), 1.54 (d, J = 6.8 Hz, |
| | | | | 3H), 1.24 (d, J = 6.9 Hz, 3H), 0.91 (s, 9H) |
| I-168^{f, h} | KF | KC | 950.6 | 0.91 (s, 9 H) 1.54 (d, 7 = 6.63 Hz, 3 H) 1.56-1.64 (m, 2 H) 1.66- |
| | | | | 1.80 (m, 5 H) 1.87-2.04 (m, 4 H) 2.25 (br t, 7 = 8.57 Hz, 2 H) 2.31- |
| | | | | 2.41 (m, 2 H) 2.65-2.68 (m, 1 H) 2.81-2.98 (m, 5 H) 3.53-3.61 |
| | | | | (m, 5 H) 3.70-3.77 (m, 2 H) 3.81-3.89 (m, 2 H) 4.12-4.14 (m, 1 |
| | | | | H) 4.24-4.32 (m, 2 H) 4.44 (br t, 7 = 8.00 Hz, 1 H) 5.05 (br dd, |
| | | | | 7 = 13.32, 4.44 Hz, 1 H) 5.59 (d, 7 = 9.38 Hz, 1 H) 6.04 (q, 7 = 6.38 |
| | | | | Hz, 1 H) 6.94-6.98 (m, 2 H) 7.30 (d, 7 = 8.13 Hz, 3 H) 7.41 (d, |
| | | | | 7 = 8.13 Hz, 2 H) 8.19-8.23 (m, 1 H) 8.28-8.39 (m, 4 H) 8.70- |
| | | | | 8.72 (m, 1 H) 8.71 (s, 1 H) |
| I-169 | KW | KC | 964.5 | 8.72 (s, 1H), 8.38 (d, 7 = 8.0 Hz, 1H), 8.34 (s, 2H), 8.26-8.18 (m, |
| | | | | 2H), 7.45-7.39 (m, 2H), 7.34-7.26 (m, 4H), 7.00-6.93 (m, 2H), |
| | | | | 6.08-6.01 (m, 1H), 5.59 (d, 7 = 9.2 Hz, 1H), 5.13-5.02 (m, 2H), |
| | | | | 4.92-4.83 (m, 1H), 4.45-4.36 (m, 2H), 4.33-4.24 (m, 2H), 4.17- |
| | | | | 4.10 (m, 1H), 3.89-3.82 (m, 2H), 3.74 (d, J = 7.6 Hz, 2H), 3.44 |
| | | | | (s, 2H), 2.96-2.85 (m, 4H), 2.30-2.22 (m, 2H), 2.00-1.89 (m, |
| | | | | 4H), 1.86-1.66 (m, 9H), 1.64-1.50 (m, 6H), 0.92 (s, 9H) |
| I-174^{d} | HH | KR | 955.5 | 14.92-14.56 (m, 1H), 8.53 (t, J = 6.0 Hz, 1H), 7.92 (dd, J = 1.6, |
| | | | | 8.4 Hz, 1H), 7.67 (d, J = 9.2 Hz, 1H), 7.43-7.27 (m, 5H), 7.25- |
| | | | | 7.16 (m, 2H), 6.91-6.80 (m, 2H), 5.11 (d, J = 3.6 Hz, 1H), 4.49 (d, |
| | | | | J = 9.6 Hz, 1H), 4.45-4.31 (m, 3H), 4.23-4.11 (m, 2H), 4.04 (d, J = |
| | | | | 12.0 Hz, 1H), 3.70-3.57 (m, 2H), 3.52-3.43 (m, 1H), 3.17 (d, J = |
| | | | | 9.2 Hz, 2H), 3.05 (d, J = 7.2 Hz, 2H), 2.89 (d, J = 10.4 Hz, 2H), |
| | | | | 2.82 (d, J = 11.2 Hz, 3H), 2.36-2.26 (m, 2H), 2.22-1.99 (m, 4H), |
| | | | | 1.97-1.84 (m, 3H), 1.76 (d, J = 7.6 Hz, 3H), 1.71-1.55 (m, 6H), |
| | | | | 1.53-1.32 (m, 10H), 1.31-1.19 (m, 3H), 0.94-0.94 (m, 1H), 0.93 |
| | | | | (s, 8H) |
| I-175^{d} | HH | KR | 956.1 | 14.79-14.66 (m, 1H), 14.85-14.63 (m, 1H), 8.57-8.49 (m, 1H), |
| | | | | 7.95-7.87 (m, 1H), 7.73-7.65 (m, 1H), 7.38-7.35 (m, 2H), 7.33- |
| | | | | 7.30 (m, 2H), 7.24-7.16 (m, 2H), 6.90-6.79 (m, 2H), 5.11 (d, J = |
| | | | | 3.6 Hz, 1H), 4.53-4.46 (m, 1H), 4.45-4.30 (m, 3H), 4.25-4.11 |
| | | | | (m, 2H), 4.09-4.00 (m, 1H), 3.71-3.56 (m, 2H), 3.53-3.42 (m, |
| | | | | 1H), 3.13 (s, 4H), 3.09-3.00 (m, 3H), 2.97-2.77 (m, 4H), 2.31- |
| | | | | 2.14 (m, 5H), 2.09-1.96 (m, 3H), 1.95-1.75 (m, 7H), 1.74-1.63 |
| | | | | (m, 3H), 1.59-1.37 (m, 10H), 1.32-1.17 (m, 4H), 1.16-1.13 (m, |
| | | | | 1H), 0.92 (s, 8H) |
| I-176 | MJ | IN | 973.6 | 14.81-14.70 (m, 1H), 8.65-8.48 (m, 1H), 7.86 (s, 1H), 7.74 (s, |
| | | | | 1H), 7.54-7.45 (m, 1H), 7.36-7.26 (m, 2H), 7.23-7.19 (m, 2H), |
| | | | | 7.24-7.12 (m, 3H), 6.84-6.84 (m, 1H), 6.85 (d, J = 1.2 Hz, 1H), |
| | | | | 6.85-6.84 (m, 1H), 6.85-6.83 (m, 1H), 6.85-6.83 (m, 1H), 6.83- |
| | | | | 6.83 (m, 1H), 6.85-6.83 (m, 1H), 5.12 (d, J = 3.6 Hz, 1H), 4.48 |
| | | | | (d, J = 9.6 Hz, 1H), 4.43-4.37 (m, 1H), 4.35-4.32 (m, 1H), 4.27- |
| | | | | 4.26 (m, 1H), 4.25-4.22 (m, 1H), 4.07-4.00 (m, 1H), 3.68-3.56 |
| | | | | (m, 2H), 3.46-3.46 (m, 1H), 3.44 (d, J = 2.0 Hz, 1H), 3.20-3.13 |
| | | | | (m, 2H), 3.09-3.00 (m, 2H), 2.93-2.76 (m, 5H), 2.68-2.66 (m, |
| | | | | 1H), 2.66-2.65 (m, 1H), 2.66-2.65 (m, 1H), 2.56-2.53 (m, 1H), |
| | | | | 2.34-2.27 (m, 2H), 2.22-2.15 (m, 2H), 2.12 (s, 2H), 2.04-1.97 |
| | | | | (m, 1H), 1.96-1.90 (m, 1H), 1.91-1.87 (m, 1H), 1.90-1.85 (m, |
| | | | | 1H), 1.87-1.82 (m, 1H), 1.93 (d, J = 9.6 Hz, 1H), 1.87-1.81 (m, |
| | | | | 1H), 1.79-1.71 (m, 3H), 1.69-1.64 (m, 2H), 1.60-1.54 (m, 2H), |
| | | | | 1.50-1.45 (m, 2H), 1.44-1.32 (m, 6H), 1.30-1.25 (m, 2H), 1.24- |
| | | | | 1.21 (m, 1H), 1.08-1.03 (m, 1H), 0.90 (s, 9H) |
| I-177^{d} | HP | MK | 985.5 | 8.46-8.35 (m, 1H), 8.21 (s, 1H), 7.91 (br d, J = 7.3 Hz, 1H), 7.69 |
| | | | | (br d, J = 9.1 Hz, 1H), 7.39-7.29 (m, 2H), 7.26-7.15 (m, 2H), 7.02 |
| | | | | (s, 1H), 6.93 (d, J = 7.6 Hz, 1H), 6.88-6.77 (m, 2H), 4.49 (br d, J = |
| | | | | 9.4 Hz, 1H), 4.42 (br t, J = 7.9 Hz, 1H), 4.33 (br s, 1H), 4.26 (br |
| | | | | dd, J = 6.2, 16.7 Hz, 1H), 4.15-4.11 (m, 2H), 4.11-4.00 (m, 2H), |
| | | | | 3.81 (s, 3H), 3.62 (br d, J = 6.0 Hz, 2H), 3.19-3.12 (m, 3H), 3.04 |
| | | | | (br d, J = 8.1 Hz, 3H), 2.93 (br d, J = 9.6 Hz, 2H), 2.84 (br d, J = |
| | | | | 8.8 Hz, 3H), 2.63 (br d, J = 7.9 Hz, 1H), 2.30-2.23 (m, 3H), 2.18- |
| | | | | 2.12 (m, 2H), 2.04-1.98 (m, 1H), 1.94 (br s, 1H), 1.91 (br d, J = |
| | | | | 4.9 Hz, 1H), 1.90-1.85 (m, 1H), 1.76 (br d, J = 7.4 Hz, 3H), 1.69 |
| | | | | (br d, J = 10.5 Hz, 4H), 1.54 (br s, 1H), 1.52 (br d, J = 1.3 Hz, 1H), |
| | | | | 1.50 (br s, 1H), 1.48 (br s, 1H), 1.45 (br s, 1H), 1.42 (br s, 2H), 1.40 |
| | | | | (br s, 2H), 1.27 (br d, J = 4.6 Hz, 2H), 1.25-1.19 (m, 1H), 1.16- |
| | | | | 1.05 (m, 1H), 0.97 (s, 1H), 0.90 (s, 9H) |
| I-178^{d, i} | HP | IN | 493.4 | 8.45-8.37 (m, 1H), 8.25 (s, 1H), 7.91 (dd, J = 1.4, 8.4 Hz, 1H), |
| | | | | 7.69 (br d, J = 9.3 Hz, 1H), 7.38-7.30 (m, 2H), 7.24-7.15 (m, 2H), |
| | | | | 7.02 (d, J = 1.1 Hz, 1H), 6.96-6.90 (m, 1H), 6.87-6.80 (m, 2H), |
| | | | | 4.48 (br d, J = 9.3 Hz, 1H), 4.42 (br t, J = 8.0 Hz, 1H), 4.33 (br s, |
| | | | | 1H), 4.29-4.21 (m, 1H), 4.14-4.12 (m, 1H), 4.09 (br d, J = 5.3 |
| | | | | Hz, 1H), 4.04 (br d, J = 12.0 Hz, 1H), 3.81 (s, 3H), 3.63 (br d, J = |
| | | | | 4.0 Hz, 2H), 3.20-3.14 (m, 3H), 3.06-3.00 (m, 2H), 2.91 (br d, J = |
| | | | | 9.8 Hz, 2H), 2.86-2.78 (m, 3H), 2.62-2.56 (m, 1H), 2.31-2.24 |
| | | | | (m, 2H), 2.22 (br s, 2H), 2.17-2.09 (m, 2H), 2.00 (br d, J = 8.3 Hz, |
| | | | | 1H), 1.95-1.88 (m, 2H), 1.88-1.83 (m, 1H), 1.75 (br s, 3H), 1.68 |
| | | | | (br d, J = 11.0 Hz, 5H), 1.62 (br s, 1H), 1.59 (br d, J = 5.3 Hz, 1H), |
| | | | | 1.53-1.46 (m, 2H), 1.41 (br s, 4H), 1.39-1.32 (m, 3H), 1.30-1.21 |
| | | | | (m, 3H), 0.91 (s, 9H) |
| I-211^{d} | MT | HL | 953.6 | 14.77 (br s, 1H), 8.76 (s, 1H), 7.91 (br d, J = 7.3 Hz, 1H), 7.70 (br |
| | | | | d, J = 9.5 Hz, 1H), 7.34 (br d, J = 2.4 Hz, 1H), 7.29-7.24 (m, 2H), |
| | | | | 7.23-7.17 (m, 4H), 6.87-6.81 (m, 2H), 5.13 (d, J = 3.1 Hz, 1H), |
| | | | | 4.50 (br d, J = 9.3 Hz, 1H), 4.37-4.31 (m, 2H), 4.09 (s, 1H), 4.03 |
| | | | | (br d, J = 11.3 Hz, 1H), 3.62 (br s, 2H), 3.18-3.12 (m, 3H), 3.06- |
| | | | | 3.00 (m, 2H), 2.88 (br d, J = 10.0 Hz, 2H), 2.84-2.70 (m, 3H), 2.44 |
| | | | | (br d, J = 7.4 Hz, 1H), 2.31-2.25 (m, 1H), 2.22-2.09 (m, 5H), 2.07 |
| | | | | (br s, 1H), 2.04 (br d, J = 7.8 Hz, 1H), 2.00 (br s, 1H), 1.99-1.94 |
| | | | | (m, 3H), 1.93-1.88 (m, 1H), 1.87-1.80 (m, 1H), 1.75 (br d, J = |
| | | | | 9.3 Hz, 3H), 1.63 (br d, J = 6.6 Hz, 3H), 1.61-1.54 (m, 2H), 1.42- |
| | | | | 1.30 (m, 4H), 1.26-1.21 (m, 1H), 1.21-1.13 (m, 3H), 1.11 (br d, |
| | | | | J = 7.5 Hz, 1H), 0.90 (s, 9H) |
| I-220^{d} | HN | OJ | 1002.2 | 8.64 (t, J = 6.0 Hz, 1H), 7.91 (dd, J = 1.6, 8.4 Hz, 1H), 7.70 (d, J = |
| | | | | 9.2 Hz, 1H), 7.59-7.53 (m, 2H), 7.35 (d, J = 3.2 Hz, 1H), 7.30 (dd, |
| | | | | J = 1.2, 8.0 Hz, 1H), 7.22-7.17 (m, 2H), 6.87-6.82 (m, 2H), 5.16- |
| | | | | 5.11 (m, 1H), 4.50-4.34 (m, 4H), 4.29 (s, 1H), 4.24-4.16 (m, |
| | | | | 1H), 4.04 (d, J = 12.0 Hz, 1H), 3.67-3.59 (m, 2H), 3.50-3.45 (m, |
| | | | | 1H), 3.21-3.11 (m, 3H), 3.07 (s, 2H), 2.97 (s, 2H), 2.92-2.86 (m, |
| | | | | 2H), 2.84-2.79 (m, 1H), 2.62-2.58 (m, 2H), 2.31-2.25 (m, 1H), |
| | | | | 2.20-2.13 (m, 2H), 2.06-2.00 (m, 1H), 1.93-1.83 (m, 6H), 1.76- |
| | | | | 1.61 (m, 5H), 1.56-1.43 (m, 8H), 1.40-1.32 (m, 2H), 1.29-1.22 |
| | | | | (m, 3H), 1.09-1.01 (m, 2H), 0.90 (s, 9H) |
| I-221^{d, e} | HN | MK | 495.6 | 8.64 (t, J = 5.7 Hz, 1H), 8.30 (s, 2H), 7.94-7.90 (m, 1H), 7.69 (d, |
| | | | | J = 9.4 Hz, 1H), 7.57 (d, J = 8.3 Hz, 1H), 7.55 (d, J = 1.3 Hz, 1H), |
| | | | | 7.34 (s, 1H), 7.30-7.27 (m, 1H), 7.22-7.18 (m, 2H), 6.87-6.83 |
| | | | | (m, 2H), 4.49 (d, J = 9.4 Hz, 1H), 4.46-4.40 (m, 2H), 4.38-4.34 |
| | | | | (m, 2H), 4.29 (s, 1H), 4.23 (d, J = 4.3 Hz, 1H), 4.21-4.17 (m, 1H), |
| | | | | 4.07-4.02 (m, 2H), 3.19-3.16 (m, 3H), 3.06-3.02 (m, 3H), 2.95- |
| | | | | 2.90 (m, 3H), 2.82 (d, J = 10.4 Hz, 4H), 2.55 (d, J = 1.1 Hz, 1H), |
| | | | | 2.53 (s, 1H), 2.32-2.28 (m, 2H), 2.25-2.21 (m, 2H), 2.18-2.10 |
| | | | | (m, 3H), 2.07-2.00 (m, 2H), 1.96-1.89 (m, 3H), 1.76 (s, 2H), 1.71- |
| | | | | 1.67 (m, 4H), 1.50 (d, J = 9.5 Hz, 2H), 1.42 (s, 4H), 1.27 (d, J = |
| | | | | 9.4 Hz, 2H), 0.91 (s, 9H) |
| I-222^{d, e} | HN | IN | 495.6 | 8.64 (t, J = 6 Hz, 1H), 8.19 (s, 1H), 7.92 (d, J = 7.6 Hz, 1H), 7.70 |
| | | | | (d, J = 9.6 Hz, 1H), 7.57 (d, J = 8.0 Hz, 1H), 7.55 (d, J = 1.6 Hz, |
| | | | | 1H), 7.34 (d, J = 3.6 Hz, 1H), 7.32-7.29 (m, 1H), 7.23-7.19 (m, |
| | | | | 2H), 6.88-6.81 (m, 2H), 5.14 (d, J = 2.8 Hz, 1H), 4.50 (d, J = 9.6 |
| | | | | Hz, 1H), 4.48-4.39 (m, 2H), 4.38-4.33 (m, 2H), 4.29 (s, 1H), 4.24- |
| | | | | 4.18 (m, 1H), 4.05 (d, J = 11.6 Hz, 1H), 3.63 (d, J = 5.5 Hz, 2H), |
| | | | | 3.18 (d, J = 3.8 Hz, 3H), 3.08-3.02 (m, 3H), 3.00-2.93 (m, 3H), |
| | | | | 2.91-2.81 (m, 4H), 2.58-2.57 (m, 1H), 2.53 (s, 1H), 2.29 (d, J = |
| | | | | 10.4 Hz, 2H), 2.24-2.18 (m, 2H), 2.06-1.98 (m, 2H), 1.94-1.87 |
| | | | | (m, 2H), 1.79 (d, J = 6.4 Hz, 3H), 1.72 (s, 3H), 1.55 (d, J = 9.6 Hz, |
| | | | | 2H), 1.51-1.46 (m, 3H), 1.46-1.41 (m, 4H), 1.31-1.26 (m, 2H), |
| | | | | 1.24 (s, 1H), 1.19-1.13 (m, 1H), 0.91 (s, 9H) |
| I-223^{d} | HH | OJ | 967.7 | 8.58-8.51 (m, 1H), 7.94-7.88 (m, 1H), 7.71 (br d, J = 10.0 Hz, |
| | | | | 1H), 7.39-7.30 (m, 5H), 7.24-7.18 (m, 2H), 6.89-6.82 (m, 2H), |
| | | | | 5.12 (d, J = 3.6 Hz, 1H), 4.50 (d, J = 9.2 Hz, 1H), 4.44-4.32 (m, |
| | | | | 3H), 4.22-4.15 (m, 1H), 4.15-4.12 (m, 1H), 4.10-4.02 (m, 1H), |
| | | | | 3.63 (d, J = 15.6 Hz, 2H), 3.48 (m, 1H), 3.19-3.15 (m, 2H), 2.94- |
| | | | | 2.83 (m, 6H), 2.30-2.22 (m, 4H), 2.03-1.96 (m, 5H), 1.91-1.80 |
| | | | | (m, 4H), 1.78-1.66 (m, 4H), 1.63-1.46 (m, 7H), 1.32-1.19 (m, |
| | | | | 7H), 0.93 (s, 10H) |
| I-225^{d} | MT | HL | 953.7 | 14.75 (br s, 1H), 8.74 (s, 1H), 7.91 (d, J = 7.3 Hz, 1H), 7.67 (br d, |
| | | | | J = 9.4 Hz, 1H), 7.32 (d, J = 3.4 Hz, 1H), 7.29-7.24 (m, 2H), 7.23- |
| | | | | 7.17 (m, 4H), 6.87-6.81 (m, 2H), 5.11 (d, J = 3.4 Hz, 1H), 4.50 |
| | | | | (d, J = 9.3 Hz, 1H), 4.37-4.31 (m, 2H), 4.08 (s, 1H), 4.06-4.00 |
| | | | | (m, 1H), 3.62 (br s, 2H), 3.50-3.43 (m, 1H), 3.20-3.08 (m, 4H), |
| | | | | 3.04 (br d, J = 8.0 Hz, 3H), 2.91-2.82 (m, 3H), 2.79-2.72 (m, 2H), |
| | | | | 2.45 (br d, J = 7.8 Hz, 1H), 2.31-2.24 (m, 1H), 2.18-2.12 (m, 3H), |
| | | | | 2.11-2.03 (m, 5H), 1.99-1.90 (m, 3H), 1.87-1.79 (m, 2H), 1.78- |
| | | | | 1.71 (m, 3H), 1.68-1.60 (m, 4H), 1.59-1.55 (m, 1H), 1.41-1.30 |
| | | | | (m, 4H), 1.23 (s, 1H), 1.13-1.08 (m, 1H), 0.90 (s, 9H) |
| I-229^{d} | HN | OL | 961.6 | 8.65 (br t, J = 5.8 Hz, 1H), 8.18 (s, 1H), 7.91 (br d, J = 7.4 Hz, 1H), |
| | | | | 7.66 (br d, J = 9.1 Hz, 1H), 7.58-7.52 (m, 2H), 7.34 (br d, J = 2.5 |
| | | | | Hz, 1H), 7.31-7.27 (m, 1H), 7.23-7.17 (m, 2H), 6.87-6.81 (m, |
| | | | | 2H), 5.29-4.94 (m, 1H), 4.50 (br d, J = 9.4 Hz, 1H), 4.45-4.36 |
| | | | | (m, 2H), 4.35 (br s, 1H), 4.30 (s, 1H), 4.23-4.16 (m, 1H), 4.06- |
| | | | | 4.01 (m, 1H), 3.64 (br s, 2H), 3.19-3.14 (m, 3H), 3.12 (br d, J = |
| | | | | 8.4 Hz, 1H), 3.04 (br d, J = 7.3 Hz, 3H), 2.94 (br d, J = 9.0 Hz, 2H), |
| | | | | 2.88-2.75 (m, 4H), 2.31-2.22 (m, 3H), 2.19 (br d, J = 3.9 Hz, 1H), |
| | | | | 2.17 (br s, 1H), 2.14 (br d, J = 2.0 Hz, 1H), 2.10 (br d, J = 7.3 Hz, |
| | | | | 1H), 2.07 (br d, J = 2.3 Hz, 1H), 2.06-2.02 (m, 1H), 2.01 (br s, |
| | | | | 1H), 1.95 (br d, J = 8.8 Hz, 1H), 1.91 (br d, J = 4.1 Hz, 1H), 1.88 |
| | | | | (br s, 1H), 1.86 (br d, J = 3.5 Hz, 1H), 1.77 (br d, J = 9.6 Hz, 3H), |
| | | | | 1.71 (br s, 2H), 1.69 (br s, 2H), 1.40 (br s, 4H), 0.89 (s, 9H) |
| I-230^{d} | HN | OM | 961.6 | 8.65 (br t, J = 6.0 Hz, 1H), 8.18 (s, 1H), 7.91 (br d, J = 7.4 Hz, 1H), |
| | | | | 7.68 (br d, J = 9.3 Hz, 1H), 7.58-7.53 (m, 2H), 7.35 (br d, J = 2.8 |
| | | | | Hz, 1H), 7.31-7.27 (m, 1H), 7.21 (s, 1H), 7.20 (br s, 1H), 6.87- |
| | | | | 6.82 (m, 2H), 5.27-5.00 (m, 1H), 4.50 (d, J = 9.3 Hz, 1H), 4.46- |
| | | | | 4.36 (m, 2H), 4.35 (br s, 1H), 4.30 (s, 1H), 4.20 (br dd, J = 5.4, 16.6 |
| | | | | Hz, 1H), 4.04 (br d, J = 11.8 Hz, 1H), 3.64 (br s, 2H), 3.17 (br d, J = |
| | | | | 6.6 Hz, 3H), 3.13 (br d, J = 8.3 Hz, 1H), 3.05 (br s, 1H), 3.03 (br |
| | | | | s, 2H), 2.95 (br s, 1H), 2.93 (br s, 1H), 2.85-2.78 (m, 3H), 2.54 (br |
| | | | | s, 1H), 2.29 (br s, 1H), 2.27 (br d, J = 1.9 Hz, 1H), 2.24 (br s, 1H), |
| | | | | 2.20 (br s, 1H), 2.17 (br s, 1H), 2.16 (br s, 2H), 2.07-2.02 (m, 2H), |
| | | | | 2.00 (br s, 1H), 1.98 (br s, 1H), 1.96 (br d, J = 2.8 Hz, 1H), 1.91 (br |
| | | | | s, 1H), 1.89-1.85 (m, 1H), 1.77 (br d, J = 10.0 Hz, 3H), 1.70 (br d, |
| | | | | J = 6.5 Hz, 2H), 1.64 (br s, 2H), 1.45-1.36 (m, 4H), 0.89 (s, 9H) |
| I-231^{d} | HP | OL | 957.3 | 8.45-8.39 (m, 1H), 8.18 (br d, J = 8.8 Hz, 1H), 7.91 (br d, J = 7.5 |
| | | | | Hz, 1H), 7.66 (br d, J = 9.1 Hz, 1H), 7.38-7.32 (m, 2H), 7.21 (s, |
| | | | | 1H), 7.21-7.16 (m, 1H), 7.02 (s, 1H), 6.92 (d, J = 7.6 Hz, 1H), 6.86- |
| | | | | 6.84 (m, 1H), 6.83 (s, 1H), 5.23-4.99 (m, 1H), 4.50 (br d, J = 9.4 |
| | | | | Hz, 1H), 4.41 (br t, J = 8.3 Hz, 1H), 4.33 (br s, 1H), 4.25 (br dd, J = |
| | | | | 6.3, 16.8 Hz, 1H), 4.14 (s, 1H), 4.12-4.07 (m, 1H), 4.04 (br d, J = |
| | | | | 11.0 Hz, 1H), 3.81 (s, 3H), 3.63 (br s, 2H), 3.46 (br d, J = 4.5 Hz, |
| | | | | 2H), 3.17 (br d, J = 3.3 Hz, 2H), 3.15 (br s, 1H), 3.12 (br d, J = 8.0 |
| | | | | Hz, 1H), 3.06-3.03 (m, 1H), 3.01 (br d, J = 2.5 Hz, 2H), 2.96 (br |
| | | | | s, 1H), 2.88-2.80 (m, 3H), 2.54 (s, 1H), 2.29 (br d, J = 1.3 Hz, 1H), |
| | | | | 2.26 (br s, 3H), 2.20-2.12 (m, 2H), 2.09 (br d, J = 4.0 Hz, 1H), |
| | | | | 2.08-2.03 (m, 2H), 2.02-1.96 (m, 2H), 1.91 (br d, J = 3.9 Hz, 1H), |
| | | | | 1.89-1.86 (m, 1H), 1.85 (br s, 1H), 1.80 (br s, 2H), 1.77 (br s, 2H), |
| | | | | 1.73 (br s, 2H), 1.44 (br d, J = 3.3 Hz, 4H), 0.89 (s, 9H) |
| I-232^{d} | HP | OM | 957.5 | 8.44-8.39 (m, 1H), 8.18 (s, 1H), 7.91 (br d, J = 7.1 Hz, 1H), 7.66 |
| | | | | (br d, J = 9.3 Hz, 1H), 7.37-7.32 (m, 2H), 7.21 (s, 1H), 7.20-7.17 |
| | | | | (m, 1H), 7.02 (s, 1H), 6.92 (d, J = 7.5 Hz, 1H), 6.86-6.84 (m, 1H), |
| | | | | 6.83 (s, 1H), 5.24-4.98 (m, 1H), 4.50 (d, J = 9.4 Hz, 1H), 4.41 (t, |
| | | | | J = 8.0 Hz, 1H), 4.33 (br s, 1H), 4.29-4.22 (m, 1H), 4.14 (s, 1H), |
| | | | | 4.12-4.07 (m, 1H), 4.04 (br d, J = 11.4 Hz, 1H), 3.81 (s, 3H), 3.63 |
| | | | | (br s, 2H), 3.46 (br s, 1H), 3.16 (br d, J = 7.5 Hz, 2H), 3.13 (br s, |
| | | | | 1H), 3.12-3.08 (m, 1H), 3.03 (br d, J = 7.3 Hz, 2H), 2.96 (br d, J = |
| | | | | 10.9 Hz, 2H), 2.83 (br t, J = 10.6 Hz, 3H), 2.54 (br s, 1H), 2.29 |
| | | | | (br s, 1H), 2.27-2.24 (m, 1H), 2.22 (br s, 1H), 2.19 (br d, J = 8.0 |
| | | | | Hz, 2H), 2.17-2.12 (m, 2H), 2.03 (br s, 2H), 1.99 (br d, J = 2.3 Hz, |
| | | | | 1H), 1.97 (br s, 1H), 1.95 (br d, J = 2.9 Hz, 1H), 1.92-1.84 (m, |
| | | | | 2H), 1.79 (br s, 2H), 1.77 (br s, 2H), 1.73 (br d, J = 0.9 Hz, 1H), |
| | | | | 1.70 (br s, 1H), 1.66 (br s, 1H), 1.41 (br d, J = 10.5 Hz, 4H), 0.89 |
| | | | | (s, 9H) |
| I-235 | ON | KC | 962.5 | 8.8 (s, 1H), 8.6 (d, J = 8.6 Hz, 1H), 8.34 (s, 2H), 8.23-8.20 (m, |
| | | | | 1H), 7.34-7.27 (m, 2H), 6.99-6.94 (m, 2H), 6.88 (d, J = 1.5, 7.9 |
| | | | | Hz, 1H), 6.86-6.82 (m, 1H), 6.04 (d, J = 6.3 Hz, 1H), 5.71 (d, J = |
| | | | | 9.4 Hz, 1H), 5.08-4.99 (m, 2H), 4.38-4.30 (m, 3H), 4.27-4.17 |
| | | | | (m, 2H), 4.15-4.12 (m, 1H), 3.90-3.80 (m, 2H), 3.78-3.69 (m, |
| | | | | 3H), 3.67-3.63 (m, 2H), 3.46-3.39 (m, 3H), 3.00-2.90 (m, 2H), |
| | | | | 2.85 (d, J = 10.8 Hz, 2H), 2.29-2.21 (m, 2H), 2.05-1.97 (m, 2H), |
| | | | | 1.95-1.84 (m, 4H), 1.79-1.67 (m, 5H), 1.66-1.59 (m, 2H), 1.58- |
| | | | | 1.56 (m, 1H), 1.56-1.52 (m, 3H), 0.97 (s, 9H) |
| I-236 | OO | KC | 962.5 | 0.92 (s, 9 H) 1.53 (br d, J = 6.50 Hz, 3 H) 1.56-1.62 (m, 2 H) 1.66- |
| | | | | 1.79 (m, 5 H) 1.87-2.02 (m, 7 H) 2.24 (br s, 3 H) 2.84 (br d, |
| | | | | J = 10.13 Hz, 3 H) 2.93 (br s, 2 H) 3.70-3.75 (m, 3 H) 3.84 (br d, |
| | | | | J = 11.63 Hz, 3 H) 4.13 (s, 1 H) 4.22 (br t, J = 4.94 Hz, 2 H) 4.27- |
| | | | | 4.37 (m, 4 H) 4.91-4.96 (m, 1 H) 5.02-5.07 (m, 1 H) 5.61 (br d, |
| | | | | J = 9.26 Hz, 1 H) 6.01-6.07 (m, 1 H) 6.86 (s, 1 H) 6.96 (br d, J = 7.88 |
| | | | | Hz, 3 H) 7.09 (d, J = 8.00 Hz, 1 H) 7.29 (br t, J = 7.57 Hz, 1 H) 8.18- |
| | | | | 8.26 (m, 2 H) 8.33 (s, 2 H) 8.44 (br d, J = 8.00 Hz, 1 H) 8.71 (s, 1 H) |
| I-237^{d} | HH | OJ | 967.8 | 8.57-8.52 (m, 1H), 7.91 (d, J = 7.6 Hz, 1H), 7.68 (d, J = 9.0 Hz, |
| | | | | 1H), 7.39-7.31 (m, 5H), 7.23-7.18 (m, 2H), 6.88-6.83 (m, 2H), |
| | | | | 5.11 (d, J = 3.1 Hz, 1H), 4.49 (d, J = 9.2 Hz, 1H), 4.39 (t, J = 8.0 |
| | | | | Hz, 2H), 4.34 (d, J = 2.0 Hz, 1H), 4.22-4.12 (m, 2H), 4.09-4.02 |
| | | | | (m, 1H), 3.69-3.60 (m, 2H), 3.49-3.46 (m, 1H), 3.19-3.15 (m, |
| | | | | 2H), 3.08 (m, 2H), 2.97 (m, 2H), 2.89 (dd, J = 4.0, 6.0 Hz, 2H), |
| | | | | 2.20-2.14 (m, 3H), 1.93-1.85 (m, 5H), 1.72-1.63 (m, 4H), 1.57- |
| | | | | 1.37 (m, 10H), 1.31-1.21 (m, 5H), 1.09-1.02 (m, 2H), 0.93 (s, 9H) |
| I-238^{d} | HN | OJ | 1002.2 | 8.64 (t, J = 5.6 Hz, 1H), 7.91 (d, J = 7.2 Hz, 1H), 7.70 (d, J = 9.2 |
| | | | | Hz, 1H), 7.60-7.51 (m, 2H), 7.35 (d, J = 3.2 Hz, 1H), 7.30 (dd, J = |
| | | | | 1.6, 8.0 Hz, 1H), 7.23-7.17 (m, 2H), 6.88-6.81 (m, 2H), 5.13 |
| | | | | (d, J = 3.2 Hz, 1H), 4.50-4.32 (m, 4H), 4.29 (s, 1H), 4.24-4.16 |
| | | | | (m, 1H), 4.04 (d, J = 11.2 Hz, 1H), 3.67-3.58 (m, 2H), 3.49-3.45 |
| | | | | (m, 1H), 3.16 (td, J = 9.2, 18.4 Hz, 3H), 3.08 (s, 2H), 2.97 (s, 2H), |
| | | | | 2.92-2.86 (m, 2H), 2.84-2.78 (m, 1H), 2.63-2.57 (m, 2H), 2.30- |
| | | | | 2.24 (m, 1H), 2.16 (d, J = 7.6 Hz, 2H), 2.01 (d, J = 8.4 Hz, 1H), |
| | | | | 1.94-1.84 (m, 6H), 1.64 (dd, J = 10.4, 11.2 Hz, 5H), 1.57-1.32 |
| | | | | (m, 10H), 1.29-1.20 (m, 3H), 1.09-1.00 (m, 2H), 0.90 (s, 9H) |
| I-239^{d} | HH | PN | 887.7 | 14.73 (s, 1H), 8.53 (t, J = 6.0 Hz, 1H), 7.92 (d, J = 7.6 Hz, 1H), 7.63 |
| | | | | (d, J = 9.6 Hz, 1H), 7.35 (s, 2H), 7.33-7.29 (m, 2H), 7.24 (s, 1H), |
| | | | | 7.20 (t, J = 7.6 Hz, 1H), 6.88-6.83 (m, 2H), 5.11 (d, J = 3.2 Hz, |
| | | | | 1H), 4.52-4.47 (m, 1H), 4.43-4.35 (m, 2H), 4.35-4.30 (m, 1H), |
| | | | | 4.12 (s, 2H), 4.08-4.02 (m, 1H), 3.66-3.59 (m, 2H), 3.55-3.42 |
| | | | | (m, 5H), 3.23 (d, J = 10.0 Hz, 3H), 3.18 (s, 2H), 3.00-2.93 (m, |
| | | | | 2H), 2.92-2.84 (m, 1H), 2.54 (s, 1H), 2.31-2.26 (m, 1H), 2.24- |
| | | | | 2.14 (m, 5H), 2.12-2.03 (m, 4H), 2.01-1.92 (m, 3H), 1.90-1.85 |
| | | | | (m, 2H), 1.81-1.75 (m, 1H), 1.71-1.64 (m, 1H), 0.90 (s, 9H) |
| I-240^{d} | HH | PO | 887.3 | 14.73 (s, 1H), 8.53 (s, 1H), 7.92 (d, J = 7.2 Hz, 1H), 7.68-7.61 (m, |
| | | | | 1H), 7.35 (s, 2H), 7.33-7.29 (m, 2H), 7.24 (s, 1H), 7.22-7.18 (m, |
| | | | | 1H), 6.87-6.82 (m, 2H), 5.11 (d, J = 3.6 Hz, 1H), 4.49 (d, J = 9.6 |
| | | | | Hz, 1H), 4.43-4.35 (m, 2H), 4.35-4.30 (m, 1H), 4.20-4.11 (m, |
| | | | | 2H), 4.09-4.02 (m, 1H), 3.67-3.59 (m, 2H), 3.53-3.40 (m, 5H), |
| | | | | 3.28-3.21 (m, 3H), 3.14 (d, J = 8.4 Hz, 2H), 2.96 (d, J = 7.2 Hz, |
| | | | | 2H), 2.88 (s, 1H), 2.54 (s, 1H), 2.31-2.25 (m, 1H), 2.25-2.10 (m, |
| | | | | 7H), 2.09-2.02 (m, 1H), 2.01-1.95 (m, 4H), 1.91 (d, J = 11.2 Hz, |
| | | | | 1H), 1.88-1.82 (m, 1H), 1.78 (t, J = 9.2 Hz, 1H), 1.70-1.62 (m, |
| | | | | 1H), 0.90 (s, 9H) |
| I-241^{d} | PI | OM | 943.5 | 8.46-8.40 (m, 1H), 8.23 (s, 1H), 7.90 (br d, J = 7.6 Hz, 1H), 7.65 |
| | | | | (br d, J = 9.1 Hz, 1H), 7.33 (br s, 1H), 7.25 (br d, J = 7.6 Hz, 1H), |
| | | | | 7.23-7.16 (m, 2H), 6.88-6.81 (m, 3H), 6.78 (br d, J = 7.8 Hz, 1H), |
| | | | | 4.49 (br d, J = 9.3 Hz, 1H), 4.40 (br t, J = 8.1 Hz, 1H), 4.33 (br s, |
| | | | | 1H), 4.23 (br dd, J = 6.3, 16.3 Hz, 2H), 4.11 (br d, J = 5.1 Hz, 1H), |
| | | | | 4.07 (br d, J = 5.3 Hz, 1H), 4.04-4.00 (m, 2H), 3.15 (br d, J = 6.1 |
| | | | | Hz, 3H), 3.11 (br d, J = 8.5 Hz, 2H), 3.01 (br d, J = 8.6 Hz, 4H), |
| | | | | 2.87 (br s, 2H), 2.84-2.79 (m, 1H), 2.67 (br t, J = 7.6 Hz, 1H), 2.54 |
| | | | | (br s, 1H), 2.41 (br d, J = 9.5 Hz, 1H), 2.27 (br s, 4H), 2.20-2.16 |
| | | | | (m, 1H), 2.14 (br d, J = 9.4 Hz, 1H), 2.07-2.03 (m, 1H), 2.01 (br |
| | | | | d, J = 1.3 Hz, 1H), 2.00-1.94 (m, 3H), 1.91-1.89 (m, 1H), 1.88 |
| | | | | (br s, 1H), 1.85 (br d, J = 8.1 Hz, 2H), 1.81 (br s, 2H), 1.79-1.70 |
| | | | | (m, 4H), 1.46 (br d, J = 9.9 Hz, 4H), 0.89 (s, 9H) |
| I-242^{d} | PI | OL | 943.4 | 8.48-8.40 (m, 1H), 8.25 (s, 1H), 7.91 (br d, J = 7.8 Hz, 1H), 7.65 |
| | | | | (br d, J = 9.1 Hz, 1H), 7.35 (br s, 1H), 7.25 (br d, J = 7.8 Hz, 1H), |
| | | | | 7.23-7.17 (m, 2H), 6.85 (br s, 2H), 6.83 (br s, 1H), 6.78 (br d, J = |
| | | | | 7.8 Hz, 1H), 4.50 (br d, J = 9.3 Hz, 1H), 4.41 (br t, J = 7.9 Hz, 1H), |
| | | | | 4.34 (br s, 1H), 4.24 (br dd, J = 6.3, 16.3 Hz, 1H), 4.09 (br dd, J = |
| | | | | 5.3, 16.5 Hz, 1H), 4.06-3.98 (m, 2H), 3.63 (br s, 2H), 3.47 (br d, J = |
| | | | | 4.6 Hz, 1H), 3.15 (br d, J = 5.9 Hz, 2H), 3.13-3.07 (m, 1H), 3.00 |
| | | | | (br s, 4H), 2.84 (br d, J = 10.9 Hz, 3H), 2.70-2.60 (m, 1H), 2.55- |
| | | | | 2.46 (m, 3H), 2.40 (br s, 1H), 2.26 (br d, J = 9.9 Hz, 4H), 2.14 (br |
| | | | | d, J = 8.6 Hz, 2H), 2.10 (br s, 1H), 2.08-2.04 (m, 1H), 2.00 (br d, |
| | | | | J = 7.6 Hz, 2H), 1.89 (br d, J = 8.0 Hz, 2H), 1.84 (br d, J = 7.0 Hz, |
| | | | | 2H), 1.81 (br s, 2H), 1.77 (br s, 4H), 1.46 (br s, 4H), 0.90 (s, 9H) |
| I-258 | MJ | MK | 973.5 | 14.76 (s, 1H), 8.56 (t, J = 6.0 Hz, 1H), 7.92 (dd, J = 1.6, 8.3 Hz, |
| | | | | 1H), 7.68 (d, J = 9.2 Hz, 1H), 7.50 (t, J = 8.0 Hz, 1H), 7.35-7.28 |
| | | | | (m, 2H), 7.25-7.15 (m, 3H), 6.88-6.81 (m, 2H), 5.12 (d, J = 3.6 |
| | | | | Hz, 1H), 4.49 (d, J = 9.2 Hz, 1H), 4.43-4.38 (m, 1H), 4.35 (d, J = |
| | | | | 6.4 Hz, 2H), 4.28-4.18 (m, 2H), 4.04 (d, J = 12.0 Hz, 1H), 3.67- |
| | | | | 3.58 (m, 2H), 3.51-3.43 (m, 1H), 3.17 (d, J = 9.2 Hz, 2H), 3.05 (d, |
| | | | | J = 8.0 Hz, 2H), 2.89 (d, J = 9.6 Hz, 2H), 2.82 (d, J = 11.2 Hz, 3H), |
| | | | | 2.58 (d, J = 1.2 Hz, 1H), 2.35-2.27 (m, 2H), 2.23-2.15 (m, 2H), |
| | | | | 2.10 (t, J = 11.2 Hz, 2H), 2.03-1.97 (m, 1H), 1.96-1.83 (m, 3H), |
| | | | | 1.81-1.70 (m, 4H), 1.70-1.54 (m, 6H), 1.48 (d, J = 8.4 Hz, 2H), |
| | | | | 1.45-1.32 (m, 8H), 1.31-1.20 (m, 3H), 0.91 (s, 9H) |
| I-266 | HH | QB | 977.2 | 0.87 (br s, 2 H) 0.92 (s, 9 H) 1.27 (br s, 3 H) 1.28-1.37 (m, 3 H) |
| | | | | 1.38-1.44 (m, 2 H) 1.47-1.63 (m, 3 H) 1.67-1.72 (m, 1 H) 1.83- |
| | | | | 1.91 (m, 2H) 1.97-2.04 (m, 2 H) 2.12-2.21 (m, 2 H) 2.78-2.89 |
| | | | | (m, 2 H) 3.13-3.24 (m, 4H) 3.56-3.72 (m, 3 H) 3.58 (br d, J = 3.75 |
| | | | | Hz, 1 H) 4.12-4.25 (m, 3 H) 4.33-4.43 (m, 3 H) 4.50 (br d, J = 9.26 |
| | | | | Hz, 1H) 4.56 (br d, J = 10.88 Hz, 1H) 4.64 (br d, J = 11.76 Hz, 1 H) |
| | | | | 5.13 (br d, J = 1.25 Hz, 1 H) 6.97 (br t, J = 7.44 Hz, 1 H) 7.05 (br d, |
| | | | | J = 8.13 Hz, 1 H) 7.09-7.12 (m, 2 H) 7.20-7.25 (m, 3 H) 7.30- |
| | | | | 7.33 (m, 2 H) 7.34-7.37 (m, 4 H) 7.50-7.60 (m, 1 H) 7.70 (br d, |
| | | | | J = 9.13 Hz, 1 H) 8.28 (s, 2 H) 8.54 (t, J = 6.19 Hz, 1 H) |
| I-267 | HH | QC | 977.7 | 0.44 (br s, 2 H) 0.64 (br s, 2 H) 0.92 (s, 8 H) 1.16-1.33 (m, 3 H) |
| | | | | 1.34-1.54 (m, 5 H) 1.59 (br d, J = 10.01 Hz, 1 H) 1.76 (br d, J = 12.01 |
| | | | | Hz, 1H) 1.80-1.92 (m, 2 H) 1.95-2.09 (m, 2 H) 2.24-2.34 (m, 1 |
| | | | | H) 2.59-2.74 (m, 2 H) 2.82 (br s, 2 H) 2.85-3.00 (m, 5 H) 3.03 |
| | | | | (br d, J = 10.13 Hz, 1 H) 3.19-3.28 (m, 2 H) 3.50-3.68 (m, 4 H) |
| | | | | 4.11-4.14 (m, 1 H) 4.14-4.23 (m, 2 H) 4.28-4.45 (m, 3 H) 4.49 |
| | | | | (d, J = 9.38 Hz, 1 H) 4.60 (br d, J = 12.63 Hz, 2 H) 5.06-5.16 (m, 1 |
| | | | | H) 6.80-6.91 (m, 2 H) 7.18-7.24 (m, 1 H) 7.32 (d, J = 7.63 Hz, 3 |
| | | | | H) 7.34-7.45 (m, 3 H) 7.68 (br d, J = 9.38 Hz, 1 H) 7.94 (d, J = 7.88 |
| | | | | Hz, 1 H) 8.19 (s, 2 H) 8.54 (br t, J = 6.07 Hz, 1 H) |
| I-270 | QG | KC | 934.7 | 14.35-14.29 (m, 1H), 12.42 (td, J = 2.4, 5.7 Hz, 1H), 8.71 (s, 1H), |
| | | | | 8.40 (br t, J = 4.4 Hz, 1H), 8.33 (s, 2H), 8.20 (br d, J = 8.0 Hz, 1H), |
| | | | | 7.35-7.24 (m, 3H), 7.22-7.16 (m, 1H), 6.96 (br d, J = 6.8 Hz, 2H), |
| | | | | 6.08-6.00 (m, 1H), 5.63 (br d, J = 9.6 Hz, 1H), 5.11-5.01 (m, 2H), |
| | | | | 4.41 (br t, J = 7.6 Hz, 1H), 4.31 (br d, J = 9.2 Hz, 3H), 4.18-4.06 |
| | | | | (m, 2H), 3.90-3.80 (m, 2H), 3.79-3.69 (m, 2H), 3.62 (br s, 2H), |
| | | | | 3.48-3.35 (m, 2H), 3.01-2.89 (m, 2H), 2.85 (br d, J = 10.8 Hz, |
| | | | | 2H), 2.36-2.36 (m, 1H), 2.36-2.31 (m, 1H), 2.25 (br s, 2H), 2.23 |
| | | | | (s, 3H), 2.04-1.97 (m, 1H), 1.95-1.85 (m, 3H), 1.78-1.67 (m, |
| | | | | 4H), 1.65-1.56 (m, 2H), 1.54 (br d, J = 6.4 Hz, 3H), 0.91 (s, 9H) |
| I-274 | HH | QQ | 901.8 | 8.54 (t, J = 6.0 Hz, 1H), 8.16 (s, 1H), 7.92 (dd, J = 1.2, 8.4 Hz, 1H), |
| | | | | 7.64 (d, J = 9.2 Hz, 1H), 7.39-7.29 (m, 5H), 7.24-7.17 (m, 2H), |
| | | | | 6.88-6.82 (m, 2H), 5.23-5.02 (m, 1H), 4.50 (d, J = 9.6 Hz, 1H), |
| | | | | 4.43-4.36 (m, 2H), 4.33 (s, 1H), 4.20-4.12 (m, 2H), 4.04 (d, J = |
| | | | | 12.0 Hz, 1H), 3.68-3.60 (m, 4H), 3.23-3.10 (m, 6H), 3.03 (d, J = |
| | | | | 10.4 Hz, 2H), 2.85 (dd, J = 9.6, 12.4 Hz, 1H), 2.65-2.56 (m, 3H), |
| | | | | 2.54 (s, 1H), 2.22-2.06 (m, 10H), 2.02-1.93 (m, 2H), 1.87 (dt, J = |
| | | | | 4.4, 8.4 Hz, 2H), 1.79 (t, J = 9.6 Hz, 2H), 1.72-1.64 (m, 1H), |
| | | | | 0.91 (s, 9H) |
| I-275 | HH | QR | 901.7 | 8.53 (t, J = 6.0 Hz, 1H), 7.92 (dd, J = 1.2, 8.4 Hz, 1H), 7.65 (d, J = |
| | | | | 9.6 Hz, 1H), 7.38-7.30 (m, 5H), 7.24-7.18 (m, 2H), 6.88-6.81 |
| | | | | (m, 2H), 5.11 (d, J = 3.6 Hz, 1H), 4.50 (d, J = 9.2 Hz, 1H), 4.43- |
| | | | | 4.33 (m, 3H), 4.21-4.11 (m, 2H), 4.05 (d, J = 11.6 Hz, 1H), 3.68- |
| | | | | 3.60 (m, 2H), 3.43 (s, 6H), 3.25-3.11 (m, 4H), 3.03 (d, J = 10.0 |
| | | | | Hz, 2H), 2.85 (dt, J = 3.2, 12.4 Hz, 1H), 2.58 (d, J = 6.4 Hz, 3H), |
| | | | | 2.54 (s, 2H), 2.20-2.12 (m, 6H), 2.05-1.96 (m, 5H), 1.89-1.83 |
| | | | | (m, 1H), 1.82-1.75 (m, 2H), 1.67 (dd, J = 8.4, 10.4 Hz, 1H), 0.91 |
| | | | | (s, 9H) |
| I-277 | HH | QU | 909.3 | 8.56 (t, J = 6.0 Hz, 1H), 8.25 (s, 1H), 7.92 (dd, J = 8.4 Hz, 1H), 7.75 |
| | | | | (d, J = 8.8 Hz, 2H), 7.55 (br d, J = 9.2 Hz, 1H), 7.40-7.37 (m, 2H), |
| | | | | 7.35-7.31 (m, 3H), 7.22-7.18 (m, 2H), 6.93 (br d, J = 9.2 Hz, 2H), |
| | | | | 6.87-6.82 (m, 2H), 4.74 (d, J = 9.2 Hz, 1H), 4.44-4.35 (m, 3H), |
| | | | | 4.23-4.17 (m, 1H), 4.13 (s, 1H), 4.04 (br d, J = 12.0 Hz, 1H), 3.89 |
| | | | | (br d, J = 12.0 Hz, 2H), 3.71 (br s, 2H), 3.46 (br s, 1H), 3.16 (br d, |
| | | | | J = 9.2 Hz, 2H), 3.08-3.00 (m, 3H), 2.93 (br d, J = 10.0 Hz, 2H), |
| | | | | 2.87-2.82 (m, 1H), 2.76 (br t, J = 12.0 Hz, 3H), 2.29 (br d, J = 13.2 |
| | | | | Hz, 1H), 2.25-2.18 (m, 1H), 2.13 (br t, J = 11.2 Hz, 2H), 2.06- |
| | | | | 2.00 (m, 1H), 1.95-1.86 (m, 2H), 1.77 (br d, J = 7.6 Hz, 4H), 1.53- |
| | | | | 1.39 (m, 4H), 1.01 (s, 9H) |
| I-282 | RD | KC | 950.4 | 10.45 (s, 1H), 9.80 (s, 1H), 8.71 (s, 1H), 8.34 (s, 2H), 8.25 (d, J = |
| | | | | 8.0 Hz, 1H), 8.09-7.94 (m, 1H), 7.36 (br t, J = 7.5 Hz, 1H), 7.14- |
| | | | | 7.11 (m, 1H), 7.04-6.98 (m, 2H), 6.88-6.85 (m, 2H), 6.07 (q, J = |
| | | | | 6.5 Hz, 1H), 5.75-5.70 (m, 1H), 5.10-5.05 (m, 2H), 4.42 (t, J = |
| | | | | 7.8 Hz, 1H), 4.30 (br d, J = 9.4 Hz, 1H), 4.27 (br s, 1H), 4.04 (s, |
| | | | | 1H), 3.93-3.90 (m, 1H), 3.89-3.83 (m, 2H), 3.82-3.78 (m, 1H), |
| | | | | 3.57 (br s, 3H), 3.48-3.44 (m, 2H), 3.42-3.40 (m, 2H), 2.99 (br s, |
| | | | | 2H), 2.82 (br d, J = 11.0 Hz, 2H), 2.78-2.72 (m, 1H), 2.54 (s, 1H), |
| | | | | 2.05-1.96 (m, 4H), 1.95-1.87 (m, 2H), 1.87-1.80 (m, 1H), 1.79- |
| | | | | 1.74 (m, 1H), 1.55 (d, J = 6.5 Hz, 4H), 1.26 (d, J = 6.9 Hz, 4H), |
| | | | | 0.92 (s, 9H) |
| I-283 | RE | RG | 998.9 | 8.53-8.10 (m, 1H), 7.94-7.83 (m, 1H), 7.46-7.34 (m, 6H), 7.25- |
| | | | | 7.19 (m, 2H), 6.90-6.83 (m, 2H), 6.11 (s, 1H), 5.12 (d, J = 2.8 |
| | | | | Hz, 1H), 4.86-4.73 (m, 1H), 4.40-4.22 (m, 4H), 4.12-4.03 (m, |
| | | | | 1H), 3.77 (s, 1H), 3.66 (d, J = 10.0 Hz, 1H), 3.47 (d, J = 4.4 Hz, |
| | | | | 3H), 3.18 (d, J = 11.2 Hz, 5H), 3.03-2.94 (m, 4H), 2.92-2.81 (m, |
| | | | | 3H), 2.28-2.19 (m, 4H), 2.17-2.10 (m, 2H), 2.07-1.97 (m, 4H), |
| | | | | 1.97-1.73 (m, 10H), 1.71-1.52 (m, 5H), 1.49-1.43 (m, 1H), 1.38 |
| | | | | (d, J = 6.8 Hz, 4H), 1.25 (d, J = 7.2 Hz, 2H), 0.96 (t, J = 6.4 Hz, |
| | | | | 3H), 0.84-0.78 (m, 3H) |
| I-285 | HH | RK | 948.7 | 14.33 (s, 1H), 12.52-12.31 (m, 1H), 8.69 (s, 1H), 8.54 (t, J = 6.0 |
| | | | | Hz, 1H), 8.24-8.19 (m, 3H), 7.63 (d, J = 9.2 Hz, 1H), 7.38-7.26 |
| | | | | (m, 5H), 6.99-6.93 (m, 2H), 5.97 (q, J = 6.4 Hz, 1H), 5.12 (d, J = |
| | | | | 3.6 Hz, 1H), 4.96 (dd, J = 5.6, 13.6 Hz, 1H), 4.52 (d, J = 9.2 Hz, |
| | | | | 1H), 4.43-4.32 (m, 3H), 4.26-4.11 (m, 2H), 3.65 (s, 2H), 3.37 (d, |
| | | | | J = 10.0 Hz, 1H), 3.19-3.12 (m, 1H), 2.98 (s, 4H), 2.95-2.83 (m, |
| | | | | 2H), 2.60 (s, 4H), 2.16 (d, J = 6.4 Hz, 1H), 2.05-1.93 (m, 2H), 1.93- |
| | | | | 1.75 (m, 4H), 1.74-1.63 (m, 4H), 1.59 (d, J = 6.4 Hz, 1H), 1.52 |
| | | | | (d, J = 6.4 Hz, 3H), 1.29-1.13 (m, 4H), 0.92 (s, 8H) |
| I-286 | RL | RN | 981.8 | 14.73 (br s, 1H), 8.35 (s, 2H), 8.18 (d, J = 1.2 Hz, 1H), 8.12 (t, J = |
| | | | | 5.2 Hz, 1H), 7.95 (d, J = 8.0 Hz, 1H), 7.64 (br d, J = 9.2 Hz, 1H), |
| | | | | 7.50 (d, J = 1.6 Hz, 1H), 7.41 (d, J = 3.2 Hz, 1H), 7.32 (s, 1H), 7.26- |
| | | | | 7.18 (m, 1H), 6.92-6.79 (m, 2H), 5.08 (d, J = 3.6 Hz, 1H), 4.79- |
| | | | | 4.65 (m, 2H), 4.56-4.39 (m, 4H), 4.35-4.15 (m, 3H), 3.86 (s, 3H), |
| | | | | 3.69-3.60 (m, 2H), 3.58-3.52 (m, 1H), 3.43 (br d, J = 3.2 Hz, 1H), |
| | | | | 3.27 (br d, J = 11.2 Hz, 1H), 3.18-3.09 (m, 1H), 2.96 (br d, J = 3.2 |
| | | | | Hz, 1H), 2.89 (br t, J = 10.0 Hz, 2H), 2.82-2.70 (m, 1H), 2.59 (br |
| | | | | d, J = 7.6 Hz, 1H), 2.55 (s, 1H), 2.44-2.35 (m, 1H), 2.31-2.22 (m, |
| | | | | 1H), 1.97 (br dd, J = 4.4, 7.6 Hz, 3H), 1.80 (br s, 1H), 1.69 (br s, |
| | | | | 5H), 1.61 (br d, J = 12.0 Hz, 2H), 1.50 (br d, J = 10.4 Hz, 2H), 1.46- |
| | | | | 1.36 (m, 3H), 1.31-1.23 (m, 3H), 0.92 (s, 9H) |
| I-292 | HH | RY | 910.5 | 8.60 (t, J = 5.6 Hz, 1H), 8.35 (d, J = 2.4 Hz, 1H), 8.28 (d, J = 9.6 |
| | | | | Hz, 1H), 8.19 (s, 1H), 7.92 (d, J = 7.2 Hz, 1H), 7.83 (d, J = 8.8 Hz, |
| | | | | 1H), 7.43-7.38 (m, 3H), 7.33 (d, J = 8 Hz, 3H), 7.22-7.18 (m, |
| | | | | 2H), 6.88-6.82 (m, 2H), 5.15 (s, 1H), 4.69 (d, J = 8 Hz, 1H), 4.45- |
| | | | | 4.40 (m, 1H), 4.37 (d, J = 5.6 Hz, 2H), 4.26-4.19 (m, 1H), 4.14 |
| | | | | (s, 1H), 4.04 (d, J = 12.4 Hz, 1H), 3.97 (d, J = 11.6 Hz, 2H), 3.72- |
| | | | | 3.65 (m, 2H), 3.47 (d, J = 4.4 Hz, 2H), 3.20-3.15 (m, 4H), 3.05 (t, |
| | | | | J = 7.2 Hz, 2H), 2.94 (d, J = 9.6 Hz, 2H), 2.91-2.80 (m, 4H), 2.35- |
| | | | | 2.30 (m, 1H), 2.29-2.26 (m, 1H), 2.26-2.20 (m, 1H), 2.14 (t, J = |
| | | | | 10.8 Hz, 2H), 2.09-2.02 (m, 1H), 1.97-1.88 (m, 2H), 1.84-1.75 |
| | | | | (m, 4H), 1.56-1.47 (m, 2H), 1.47-1.37 (m, 2H), 0.99 (s, 9H) |
| I-293 | HH | SA | 924.5 | 8.59-8.52 (m, 1H), 8.37-8.32 (m, 2H), 7.95 (d, J = 8.0 Hz, 1H), |
| | | | | 7.67-7.61 (m, 1H), 7.41 (d, J = 3.2 Hz, 1H), 7.39-7.35 (m, 2H), |
| | | | | 7.33 (d, J = 6.8 Hz, 3H), 7.25-7.19 (m, 1H), 6.90-6.83 (m, 2H), |
| | | | | 5.13 (d, J = 3.6 Hz, 1H), 4.77-4.67 (m, 2H), 4.52 (d, J = 9.2 Hz, |
| | | | | 1H), 4.45-4.36 (m, 2H), 4.35 (d, J = 5.2 Hz, 1H), 4.24-4.18 (m, |
| | | | | 2H), 4.17-4.13 (m, 1H), 3.67-3.59 (m, 3H), 3.18-3.09 (m, 1H), |
| | | | | 3.01-2.95 (m, 1H), 2.92 (d, J = 10.4 Hz, 2H), 2.80-2.71 (m, 1H), |
| | | | | 2.22-2.14 (m, 2H), 2.06-1.98 (m, 1H), 1.97-1.87 (m, 3H), 1.78- |
| | | | | 1.66 (m, 4H), 1.66-1.55 (m, 5H), 1.51-1.42 (m, 4H), 1.41-1.33 |
| | | | | (m, 2H), 1.24 (s, 2H), 1.20-1.14 (m, 1H), 0.95-0.91 (m, 9H) |
| I-294 | HH | SA | 924.4 | 8.56 (t, J = 6.0 Hz, 1H), 8.35 (s, 2H), 7.95 (d, J = 7.6 Hz, 1H), 7.70 |
| | | | | (d, J = 9.6 Hz, 1H), 7.41 (d, J = 3.2 Hz, 1H), 7.39-7.35 (m, 2H), |
| | | | | 7.33 (d, J = 7.2 Hz, 3H), 7.25-7.19 (m, 1H), 6.90-6.83 (m, 2H), |
| | | | | 5.13 (d, J = 3.2 Hz, 1H), 4.77-4.68 (m, 2H), 4.51 (d, J = 9.2 Hz, |
| | | | | 1H), 4.40 (t, J = 8.0 Hz, 2H), 4.34 (s, 1H), 4.24-4.16 (m, 2H), 4.14 |
| | | | | (s, 1H), 3.68-3.59 (m, 3H), 3.17-3.09 (m, 1H), 3.01-2.95 (m, |
| | | | | 1H), 2.91 (d, J = 11.2 Hz, 2H), 2.80-2.71 (m, 1H), 2.10 (d, J = 6.8 |
| | | | | Hz, 2H), 2.05-1.98 (m, 1H), 1.95-1.86 (m, 3H), 1.83-1.75 (m, |
| | | | | 3H), 1.72-1.61 (m, 5H), 1.51-1.43 (m, 1H), 1.40 (d, J = 3.2 Hz, |
| | | | | 1H), 1.38-1.30 (m, 3H), 1.29-1.26 (m, 1H), 1.26-1.21 (m, 3H), |
| | | | | 1.18 (d, J = 5.6 Hz, 1H), 0.93 (s, 9H) |
| I-296 | SC | RG | 1002.4 | 14.75 (s, 1H), 8.60-8.37 (m, 1H), 7.95-7.87 (m, 1H), 7.45-7.16 |
| | | | | (m, 6H), 6.89-6.79 (m, 2H), 6.15-5.99 (m, 1H), 5.12 (d, J = 4.0 |
| | | | | Hz, 1H), 4.45-4.16 (m, 7H), 4.03 (d, J = 11.2 Hz, 1H), 3.79-3.37 |
| | | | | (m, 4H), 3.23-3.11 (m, 2H), 3.00-2.79 (m, 9H), 2.62 (s, 2H), 2.52- |
| | | | | 2.52 (m, 2H), 2.29-2.23 (m, 1H), 2.17-2.06 (m, 7H), 2.18-2.01 |
| | | | | (m, 3H), 1.97 (s, 2H), 1.88-1.81 (m, 1H), 1.67 (d, J = 10.0 Hz, |
| | | | | 7H), 1.49 (s, 1H), 1.41-1.30 (m, 4H), 1.11-1.01 (m, 2H), 0.94 (d, |
| | | | | J = 6.4, 16.5 Hz, 3H), 0.86-0.76 (m, 3H), 0.70-0.56 (m, 1H) |
| I-298 | RL | RO | 981.5 | 8.35 (s, 2H), 8.22 (s, 1H), 8.17 (d, J = 1.6 Hz, 1H), 8.10 (br t, J = |
| | | | | 5.2 Hz, 1H), 7.94 (br d, J = 7.6 Hz, 1H), 7.62 (br d, J = 8.9 Hz, 1H), |
| | | | | 7.49 (d, J = 1.6 Hz, 1H), 7.39 (br d, J = 2.4 Hz, 1H), 7.31 (s, 1H), |
| | | | | 7.23-7.19 (m, 1H), 6.88-6.84 (m, 2H), 5.22-4.94 (m, 1H), 4.77- |
| | | | | 4.65 (m, 3H), 4.51-4.48 (m, 1H), 4.48-4.44 (m, 2H), 4.41 (s, |
| | | | | 1H), 4.31-4.27 (m, 1H), 4.24 (br d, J = 4.4 Hz, 1H), 4.20 (br dd, J = |
| | | | | 3.2, 8.4 Hz, 2H), 3.85 (s, 3H), 3.63 (br d, J = 3.6 Hz, 1H), 3.56 |
| | | | | (br s, 1H), 3.13 (br d, J = 1.2 Hz, 1H), 2.97 (br dd, J = 2.8, 12.8 Hz, |
| | | | | 1H), 2.89 (br t, J = 10.8 Hz, 3H), 2.75 (br d, J = 1.6 Hz, 1H), 2.64- |
| | | | | 2.59 (m, 1H), 2.41-2.35 (m, 1H), 2.30-2.24 (m, 1H), 1.96 (br dd, |
| | | | | J = 4.6, 7.2 Hz, 3H), 1.72 (br d, J = 4.4 Hz, 2H), 1.69 (br s, 3H), |
| | | | | 1.61 (br d, J = 12.0 Hz, 2H), 1.52 (br d, J = 8.8 Hz, 2H), 1.49 (br s, |
| | | | | 2H), 1.45-1.39 (m, 2H), 1.32-1.20 (m, 4H), 0.91 (s, 9H) |
| I-300 | HH | RK | 948.7 | 14.34 (s, 1H), 12.65-12.17 (m, 1H), 8.69 (s, 1H), 8.54 (t, J = 6.0 |
| | | | | Hz, 1H), 8.24-8.18 (m, 3H), 7.62 (d, J = 9.2 Hz, 1H), 7.39-7.26 |
| | | | | (m, 5H), 6.99-6.91 (m, 2H), 5.97 (q, J = 6.4 Hz, 1H), 5.12 (d, J = |
| | | | | 3.6 Hz, 1H), 4.96 (dd, J = 4.8, 13.6 Hz, 1H), 4.52 (d, J = 9.2 Hz, |
| | | | | 1H), 4.44-4.32 (m, 3H), 4.23-4.09 (m, 2H), 3.65 (s, 2H), 3.42- |
| | | | | 3.35 (m, 1H), 3.15 (t, J = 8.8 Hz, 1H), 2.98 (s, 5H), 2.90-2.84 (m, |
| | | | | 1H), 2.60 (s, 3H), 2.54 (s, 1H), 2.19-2.12 (m, 1H), 2.05-1.99 (m, |
| | | | | 1H), 1.92-1.75 (m, 6H), 1.70-1.57 (m, 3H), 1.52 (d, J = 6.4 Hz, |
| | | | | 3H), 1.29-1.14 (m, 4H), 0.92 (s, 8H) |
| I-303 | SF | RG | 1015.6. | 8.19-8.13 (m, 1H), 7.91 (dd, J = 1.2, 8.4 Hz, 1H), 7.52-7.47 (m, |
| | | | | 1H), 7.33 (br d, J = 3.2 Hz, 1H), 7.23-7.17 (m, 2H), 6.87-6.82 |
| | | | | (m, 2H), 6.07 (d, J = 1.6 Hz, 1H), 5.12-5.06 (m, 1H), 4.55-4.44 |
| | | | | (m, 2H), 4.43-4.40 (m, 1H), 4.35-4.26 (m, 2H), 4.25-4.16 (m, |
| | | | | 3H), 4.03 (br d, J = 11.2 Hz, 1H), 3.89-3.83 (m, 3H), 3.78-3.72 |
| | | | | (m, 1H), 3.65 (br d, J = 9.6 Hz, 1H), 3.25-3.11 (m, 3H), 3.00-2.78 |
| | | | | (m, 12H), 2.17-2.04 (m, 10H), 2.00-1.91 (m, 4H), 1.72-1.62 (m, |
| | | | | 8H), 1.42-1.33 (m, 4H), 1.09-1.03 (m, 2H), 0.97-0.91 (m, 3H), |
| | | | | 0.84-0.77 (m, 3H), 0.71-0.62 (m, 1H) |
| I-304 | SP | KC | 951.5 | 8.70 (s, 1H), 8.48 (d, J = 2.0 Hz, 1H), 8.44 (d, J = 8.0 Hz, 1H), 8.34 |
| | | | | (s, 2H), 8.23-8.19 (m, 1H), 7.74-7.69 (m, 1H), 7.52 (d, J = 8.4 |
| | | | | Hz, 1H), 7.32-7.27 (m, 1H), 6.99-6.93 (m, 2H), 6.09-6.00 (m, |
| | | | | 1H), 5.60 (d, J = 9.2 Hz, 1H), 5.11 (d, J = 3.6 Hz, 1H), 5.09-5.01 |
| | | | | (m, 1H), 4.91-4.82 (m, 2H), 4.43 (t, J = 8.0 Hz, 1H), 4.32-4.23 |
| | | | | (m, 3H), 3.89-3.81 (m, 2H), 3.78-3.70 (m, 2H), 3.67-3.53 (m, |
| | | | | 4H), 3.44-3.38 (m, 2H), 2.98-2.80 (m, 4H), 2.60-2.59 (m, 2H), |
| | | | | 2.26 (t, J = 8.0 Hz, 2H), 2.04-1.98 (m, 1H), 1.95-1.89 (m, 2H), |
| | | | | 1.78-1.68 (m, 5H), 1.65-1.57 (m, 2H), 1.54 (d, J = 6.4 Hz, 3H), |
| | | | | 0.98 (d, J = 7.2 Hz, 1H), 0.92 (s, 9H) |
| I-305 | SQ | KC | 951.5 | 8.72 (s, 1H), 8.57 (d, J = 2.0 Hz, 1H), 8.46-8.42 (m, 1H), 8.34 (s, |
| | | | | 2H), 8.24-8.19 (m, 1H), 7.85-7.81 (m, 1H), 7.44 (d, J = 8.4 Hz, |
| | | | | 1H), 7.33-7.27 (m, 1H), 7.00-6.94 (m, 2H), 6.07-6.01 (m, 1H), |
| | | | | 5.62 (d, J = 9.6 Hz, 1H), 5.12 (d, J = 3.6 Hz, 1H), 5.09-5.02 (m, |
| | | | | 1H), 4.96-4.92 (m, 1H), 4.84-4.78 (m, 1H), 4.48 (t, J = 8.0 Hz, |
| | | | | 1H), 4.36-4.32 (m, 1H), 4.29 (d, J = 9.6 Hz, 1H), 4.26 (s, 1H), 3.90- |
| | | | | 3.81 (m, 2H), 3.78-3.70 (m, 2H), 3.65-3.58 (m, 4H), 3.46-3.38 |
| | | | | (m, 2H), 2.95-2.82 (m, 4H), 2.56 (s, 2H), 2.28-2.24 (m, 2H), 2.05- |
| | | | | 2.01 (m, 1H), 1.94-1.90 (m, 2H), 1.78-1.67 (m, 5H), 1.62-1.57 |
| | | | | (m, 2H), 1.54 (d, J = 6.6 Hz, 3H), 0.93-0.89 (m, 1H), 0.84 (s, 9H) |
| I-306 | SS | SE | 1015.7 | 8.25 (s, 2H), 8.15 (s, 1H), 7.99-7.87 (m, 2H), 7.52 (d, J = 1.2 Hz, |
| | | | | 1H), 7.46-7.38 (m, 2H), 7.37-7.29 (m, 2H), 7.25-7.18 (m, 1H), |
| | | | | 6.89-6.82 (m, 2H), 5.16 (br d, J = 2.0 Hz, 1H), 4.69-4.57 (m, 2H), |
| | | | | 4.43-4.37 (m, 2H), 4.36-4.28 (m, 4H), 4.25-4.16 (m, 2H), 3.81 |
| | | | | (br d, J = 4.4 Hz, 1H), 3.66-3.56 (m, 1H), 3.50-3.44 (m, 3H), 3.28- |
| | | | | 3.27 (m, 1H), 3.02 (br d, J = 6.4 Hz, 6H), 2.35 (br d, J = 1.6 Hz, |
| | | | | 3H), 2.23-2.16 (m, 2H), 1.95 (br s, 4H), 1.84-1.71 (m, 3H), 1.68- |
| | | | | 1.60 (m, 2H), 1.58-1.41 (m, 6H), 1.31 (ddd, J = 2.4, 8.1, 13.4 Hz, |
| | | | | 2H), 1.25-1.19 (m, 3H), 0.99 (s, 6H) |
| I-307 | ST | SE | 1015.2 | 8.24 (s, 2H), 8.14-8.09 (m, 1H), 7.97-7.89 (m, 2H), 7.52 (d, J = |
| | | | | 1.6 Hz, 1H), 7.44-7.38 (m, 2H), 7.36-7.30 (m, 2H), 7.24-7.18 |
| | | | | (m, 1H), 6.89-6.83 (m, 2H), 5.16 (d, J = 4.4 Hz, 1H), 4.62 (br d, J = |
| | | | | 12.0 Hz, 2H), 4.42-4.38 (m, 2H), 4.36-4.29 (m, 4H), 4.24-4.17 |
| | | | | (m, 2H), 3.86-3.80 (m, 1H), 3.60 (br dd, J = 3.2, 11.8 Hz, 1H), |
| | | | | 3.51-3.44 (m, 3H), 3.29 (br s, 1H), 3.07-2.95 (m, 6H), 2.35 (br s, |
| | | | | 3H), 2.24 (br s, 2H), 2.10-1.94 (m, 4H), 1.78-1.66 (m, 3H), 1.62- |
| | | | | 1.54 (m, 2H), 1.53-1.42 (m, 6H), 1.33-1.26 (m, 2H), 1.25-1.19 |
| | | | | (m, 3H), 0.98 (s, 6H) |
| I-309 | TB | KC | 954.4 | 0.92 (s, 9H) 1.54 (d, J = 6.4 Hz, 3H) 1.57-1.66 (m, 2H) 1.68-2.09 |
| | | | | (m, 9 H) 2.23-2.30 (m, 2 H) 2.33-2.41 (m, 1 H) 2.83-3.00 (m, 4 |
| | | | | H) 3.40-3.45 (m, 1 H) 3.64 (br s, 2 H) 3.70-3.79 (m, 2 H) 3.81- |
| | | | | 3.90 (m, 2 H) 4.13-4.21 (m, 1 H) 4.28-4.45 (m, 4 H) 4.49-4.54 |
| | | | | (m, 1 H) 5.02-5.09 (m, 1 H) 5.12 (d, J = 3.6 Hz, 1 H) 5.61 (br d, |
| | | | | J = 9.2 Hz, 1 H) 6.01-6.01 (m, 1 H) 6.01-6.08 (m, 1 H) 6.93-6.99 |
| | | | | (m, 2 H) 7.27-7.33 (m, 2 H) 7.46-7.51 (m, 2 H) 8.21 (dd, J = 8.2, |
| | | | | 1.44 Hz, 1 H) 8.31-8.37 (m, 2 H) 8.59 (br t, J = 6.00 Hz, 1 H) 8.71 |
| | | | | (s, 1 H) 12.44 (s, 1 H) 14.25-14.39 (m, 1 H) |
| I-310 | TC | KC | 938.8 | 0.91 (br s, 9H) 1.48-1.56 (m, 3 H) 1.49-1.65 (m, 4H) 1.66-1.82 |
| | | | | (m, 5 H) 1.83-2.08 (m, 7 H) 2.21-2.28 (m, 3 H) 2.82-2.96 (m, 3 |
| | | | | H) 3.40-3.47 (m, 4 H) 3.64 (br s, 1 H) 3.66-3.92 (m, 4 H) 4.10- |
| | | | | 4.20 (m, 1 H) 4.28-4.45 (m, 4 H) 5.03-5.13 (m, 1 H) 5.57-5.66 |
| | | | | (m, 1 H) 5.97-6.13 (m, 1 H) 6.88-7.00 (m, 2 H) 7.14 (br d, J = 8.63 |
| | | | | Hz, 1 H) 7.19-7.31 (m, 2 H) 7.38-7.47 (m, 1 H) 8.20 (br d, J = 5.63 |
| | | | | Hz, 1 H) 8.33 (d, J = 2.88 Hz, 1 H) 8.57-8.64 (m, 1 H) 8.69 (br s, 1 |
| | | | | H) |
| I-319 | TY | KC | 950.8 | 14.32 (s, 1H), 12.43 (br s, 1H), 8.71 (s, 1H), 8.58-8.47 (m, 1H), |
| | | | | 8.33 (s, 2H), 8.21 (br d, J = 7.6 Hz, 1H), 7.28 (d, J = 7.6 Hz, 1H), |
| | | | | 7.02-6.93 (m, 3H), 6.89-6.83 (m, 1H), 6.04 (br d, J = 6.4 Hz, 1H), |
| | | | | 5.52 (br d, J = 10.1 Hz, 1H), 5.14-5.09 (m, 1H), 5.08-5.00 (m, |
| | | | | 1H), 4.45-4.36 (m, 2H), 4.36-4.27 (m, 2H), 4.17-4.13 (m, 1H), |
| | | | | 3.91 (s, 1H), 3.86-3.81 (m, 4H), 3.74 (br d, J = 1.2 Hz, 2H), 3.62 |
| | | | | (br d, J = 2.0 Hz, 2H), 3.44-3.38 (m, 1H), 3.29 (br s, 3H), 2.97- |
| | | | | 2.89 (m, 2H), 2.88-2.82 (m, 2H), 2.39-2.37 (m, 2H), 2.28-2.22 |
| | | | | (m, 2H), 2.06-1.98 (m, 1H), 1.97-1.80 (m, 4H), 1.78-1.68 (m, |
| | | | | 4H), 1.54 (d, J = 6.4 Hz, 3H), 0.91 (s, 9H) |
| I-320 | HH | UB | 923.7 | 8.58-8.52 (m, 1H), 7.92 (d, J = 8.0 Hz, 1H), 7.84 (s, 1H), 7.78 ( |
| | | | | d, J = 10.0 Hz, 1H), 7.50 (s, 1H), 7.37-7.30 (m, 5H), 7.25-7.17 |
| | | | | (m, 2H), 6.87-6.80 (m, 2H), 5.13 (d, J = 2.0 Hz, 1H), 4.53 (d, J = |
| | | | | 9.2 Hz, 1H), 4.44-4.32 (m, 4H), 4.21-4.15 (m, 1H), 4.14-4.11 |
| | | | | (m, 1H), 4.08-4.03 (m, 2H), 3.88 (s, 1H), 3.77 (s, 1H), 3.68-3.62 |
| | | | | (m, 2H), 3.19-3.14 (m, 3H), 3.10-3.04 (m, 2H), 2.84 (s, 1H), 2.74- |
| | | | | 2.69 (m, 1H), 2.24 (dd, J = 7.6, 12.0 Hz, 3H), 2.05-1.93 (m, 3H), |
| | | | | 1.90-1.82 (m, 3H), 1.52-1.42 (m, 2H), 0.93 (s, 9H) |
| I-321 | UV | SD | 950.1 | 14.42-14.13 (m, 1H), 12.46 (s, 1H), 8.69 (s, 1H), 8.37 (d, J = 7.6 |
| | | | | Hz, 1H), 8.32 (s, 2H), 8.09 (dd, J = 1.2, 8.4 Hz, 1H), 7.41 (d, J = |
| | | | | 8.0 Hz, 2H), 7.32-7.23 (m, 3H), 7.00-6.94 (m, 2H), 6.02 (t, J = |
| | | | | 5.6 Hz, 1H), 5.58 (br d, J = 9.6 Hz, 1H), 5.11-5.02 (m, 2H), 4.99- |
| | | | | 4.94 (m, 1H), 4.86 (quin, J = 7.2 Hz, 1H), 4.39 (t, J = 8.0 Hz, 1H), |
| | | | | 4.32-4.23 (m, 2H), 4.12 (s, 1H), 3.95 (td, J = 5.2, 10.4 Hz, 1H), |
| | | | | 3.85 (q, J = 8.0 Hz, 3H), 3.78-3.70 (m, 2H), 3.63-3.53 (m, 3H), |
| | | | | 2.99-2.84 (m, 4H), 2.54 (br s, 1H), 2.52 (br s, 2H), 2.45-2.36 (m, |
| | | | | 1H), 2.28 (br s, 2H), 2.03-1.93 (m, 3H), 1.74 (dt, J = 4.0, 8.4 Hz, |
| | | | | 3H), 1.66-1.55 (m, 2H), 1.32 (d, J = 7.2 Hz, 3H), 0.91 (s, 9H) |
| I-324 | UN | UJ | 988.4 | 8.57 (br t, J = 7.2 Hz, 1H), 8.34-7.87 (m, 1H), 7.67-7.53 (m, 1H), |
| | | | | 7.52-7.31 (m, 3H), 7.26-7.15 (m, 2H), 6.90-6.80 (m, 2H), 6.17- |
| | | | | 5.79 (m, 1H), 5.42-5.09 (m, 1H), 5.07-4.90 (m, 1H), 4.66-4.32 |
| | | | | (m, 2H), 4.31-4.22 (m, 1H), 4.06 (br d, J = 11.6 Hz, 1H), 3.77- |
| | | | | 3.59 (m, 2H), 3.54 (br d, J = 9.6 Hz, 1H), 3.48 (br dd, J = 5.2, 7.3 |
| | | | | Hz, 2H), 3.21-3.16 (m, 2H), 3.09-3.01 (m, 3H), 2.97-2.90 (m, |
| | | | | 2H), 2.90-2.82 (m, 1H), 2.74 (br s, 2H), 2.55 (s, 3H), 2.31 (br s, |
| | | | | 1H), 2.19 (br dd, J = 6.0, 8.6 Hz, 3H), 2.01-1.90 (m, 4H), 1.89- |
| | | | | 1.79 (m, 4H), 1.76-1.62 (m, 4H), 1.60-1.45 (m, 4H), 1.40-1.20 |
| | | | | (m, 5H), 1.17-1.05 (m, 2H), 1.00-0.92 (m, 2H), 0.89-0.42 (m, 6H) |
| I-325 | SR | UJ | 974.7 | 14.96-14.61 (m, 1H), 8.65-8.45 (m, 1H), 7.92 (dd, J = 1.6, 8.4 |
| | | | | Hz, 1H), 7.59-7.47 (m, 1H), 7.43-7.37 (m, 1H), 7.34 (d, J = 3.2 |
| | | | | Hz, 1H), 7.24-7.16 (m, 2H), 6.89-6.78 (m, 2H), 6.15-6.03 (m, |
| | | | | 1H), 5.14 (d, J = 2.8 Hz, 1H), 4.48-4.24 (m, 5H), 4.03 (d, J = 11.6 |
| | | | | Hz, 1H), 3.80-3.66 (m, 1H), 3.62-3.56 (m, 2H), 3.46 (dd, J = 4.0, |
| | | | | 8.0 Hz, 2H), 3.41 (d, J = 5.2 Hz, 1H), 3.16 (d, J = 8.8 Hz, 2H), 3.04 |
| | | | | (d, J = 8.4 Hz, 2H), 2.93-2.80 (m, 5H), 2.76-2.60 (m, 3H), 2.37- |
| | | | | 2.15 (m, 5H), 2.13-2.05 (m, 4H), 1.94-1.87 (m, 2H), 1.84-1.62 |
| | | | | (m, 5H), 1.39 (d, J = 10.8 Hz, 4H), 1.13-1.02 (m, 2H), 0.98-0.76 |
| | | | | (m, 6H) |
| I-327 | UO | UJ | 988.4 | 9.04-8.52 (m, 1H), 8.42-8.26 (m, 1H), 7.91 (br d, J = 7.5 Hz, 1H), |
| | | | | 7.56-7.48 (m, 1H), 7.46-7.40 (m, 1H), 7.38-7.32 (m, 2H), 7.29- |
| | | | | 7.21 (m, 2H), 7.20-7.18 (m, 1H), 6.86-6.83 (m, 2H), 6.14-6.07 |
| | | | | (m, 1H), 5.13-5.07 (m, 1H), 4.40-4.33 (m, 1H), 4.31-4.27 (m, |
| | | | | 1H), 4.25 (br d, J = 2.4 Hz, 1H), 4.03 (br d, J = 12.1 Hz, 1H), 3.68- |
| | | | | 3.65 (m, 1H), 3.56 (br s, 1H), 3.17 (br s, 1H), 2.92 (br d, J = 10.0 |
| | | | | Hz, 2H), 2.85 (br d, J = 9.9 Hz, 2H), 2.72 (br s, 2H), 2.54 (s, 3H), |
| | | | | 2.28 (br dd, J = 1.6, 10.8 Hz, 1H), 2.24-2.18 (m, 3H), 2.13 (br d, J = |
| | | | | 8.9 Hz, 2H), 2.10-2.08 (m, 2H), 2.03-1.99 (m, 1H), 1.92 (br s, |
| | | | | 1H), 1.85 (br s, 1H), 1.75 (br s, 2H), 1.72-1.66 (m, 5H), 1.45-1.36 |
| | | | | (m, 6H), 1.34-1.27 (m, 4H), 1.23 (br s, 1H), 1.12-1.07 (m, 2H), |
| | | | | 0.96-0.92 (m, 3H), 0.90-0.73 (m, 6H) |
| I-332 | QI | UU | 952.6 | 8.40 (d, J = 8.0 Hz, 1H), 8.21 (s, 1H), 7.94-7.91 (m, 1H), 7.45- |
| | | | | 7.41 (m, 2H), 7.36 (br d, J = 3.2 Hz, 1H), 7.28 (d, J = 8.0 Hz, 2H), |
| | | | | 7.23-7.20 (m, 2H), 6.88-6.83 (m, 2H), 5.83-5.80 (m, 1H), 4.87 |
| | | | | (br t, J = 7.2 Hz, 1H), 4.33 (t, J = 8.0 Hz, 1H), 4.26 (br s, 1H), 4.16- |
| | | | | 4.13 (m, 1H), 4.05 (br d, J = 11.6 Hz, 1H), 3.85 (s, 2H), 3.73 (s, |
| | | | | 2H), 3.71-3.67 (m, 1H), 3.56 (br d, J = 9.6 Hz, 1H), 3.21-3.14 |
| | | | | (m, 4H), 3.04 (br t, J = 8.0 Hz, 3H), 2.93-2.89 (m, 2H), 2.84-2.75 |
| | | | | (m, 4H), 2.31-2.24 (m, 4H), 2.20 (br dd, J = 7.1, 9.6 Hz, 3H), 2.14- |
| | | | | 2.09 (m, 2H), 1.95 (br d, J = 10.0 Hz, 4H), 1.79-1.74 (m, 3H), |
| | | | | 1.70-1.64 (m, 4H), 1.41 (br d, J = 6.4 Hz, 4H), 1.34 (br d, J = 6.8 |
| | | | | Hz, 3H), 0.95-0.92 (m, 3H), 0.79 (br s, 1H), 0.77 (d, J = 6.8 Hz, |
| | | | | 1H), 0.79-0.75 (m, 1H) |
| I-333^{c} | QI | SZ | 500.1 | 10.46-10.27 (m, 1H), 8.41 (d, J = 7.6 Hz, 1H), 8.30 (s, 1H), 7.55- |
| | | | | 7.39 (m, 4H), 7.28 (t, J = 8.4 Hz, 4H), 7.08 (d, J = 8.0 Hz, 1H), 6.99 |
| | | | | (t, J = 7.6 Hz, 1H), 6.14-5.99 (m, 1H), 4.89-4.82 (m, 1H), 4.68 |
| | | | | (s, 1H), 4.52 (s, 3H), 4.46-4.37 (m, 2H), 4.35-4.24 (m, 4H), 4.12 |
| | | | | (s, 4H), 3.70-3.63 (m, 6H), 3.57-3.43 (m, 9H), 3.34-3.24 (m, |
| | | | | 2H), 3.13-2.91 (m, 8H), 2.28 (d, J = 10.0 Hz, 5H), 2.07 (s, 1H), |
| | | | | 2.00 (d, J = 8.8 Hz, 6H), 1.74 (s, 1H), 1.49 (d, J = 10.8 Hz, 1H), |
| | | | | 1.46-1.40 (m, 1H), 1.33 (d, J = 6.8 Hz, 2H), 0.96 (d, J = 6.0 Hz, |
| | | | | 3H), 0.83 (s, 3H) |
| I-334^{c} | QI | SZ | 499.7 | 10.37-10.19 (m, 1H), 8.97 (d, J = 7.6 Hz, 1H), 8.44 (d, J = 7.2 Hz, |
| | | | | 1H), 8.27 (s, 1H), 7.51-7.38 (m, 4H), 7.38-7.25 (m, 2H), 7.23 (d, |
| | | | | J = 8.0 Hz, 2H), 7.07 (d, J = 8.4 Hz, 1H), 6.99 (t, J = 7.6 Hz, 1H), |
| | | | | 6.20-6.11 (m, 1H), 4.84-4.77 (m, 1H), 4.51 (s, 2H), 4.41 (t, J = |
| | | | | 7.6 Hz, 2H), 4.24 (s, 2H), 4.13 (s, 1H), 3.63 (s, 3H), 3.56 (d, J = |
| | | | | 10.4 Hz, 10H), 3.48-3.35 (m, 6H), 3.28 (dd, J = 6.8, 11.3 Hz, 2H), |
| | | | | 3.12-2.85 (m, 8H), 2.54 (s, 1H), 2.37-2.19 (m, 6H), 2.07 (s, 2H), |
| | | | | 2.04-1.92 (m, 5H), 1.73-1.66 (m, 1H), 1.50-1.45 (m, 1H), 1.41 |
| | | | | (d, J = 6.8 Hz, 1H), 1.32 (d, J = 6.8 Hz, 2H), 0.96 (d, J = 6.4 Hz, |
| | | | | 3H), 0.84-0.75 (m, 3H) |
| I-337 | FJ | VA | 1019.7 | 12.4 (br s, 1H), 8.58-8.50 (m, 1H), 8.33 (s, 2H), 8.26-8.14 (m, |
| | | | | 2H), 7.41-7.35 (m, 2H), 7.34-7.26 (m, 3H), 7.00-6.91 (m, 2H), |
| | | | | 6.04 (q, J = 6.4 Hz, 1H), 5.79 (br d, J = 10.0 Hz, 1H), 5.20-4.97 |
| | | | | (m, 2H), 4.46-4.30 (m, 4H), 4.19-4.10 (m, 2H), 3.89 (br d, J = |
| | | | | 8.0 Hz, 1H), 3.84-3.75 (m, 2H), 3.72-3.64 (m, 3H), 3.53 (br t, J = |
| | | | | 4.4 Hz, 4H), 2.97-2.81 (m, 4H), 2.59-2.53 (m, 2H), 2.44-2.15 |
| | | | | (m, 10H), 2.05-1.83 (m, 4H), 1.81-1.67 (m, 4H), 1.65-1.50 (m, |
| | | | | 6H), 1.43-1.32 (m, 1H), 0.92 (br d, J = 11.2 Hz, 6H) |
| I-338 | FJ | VA | 1019.2 | 12.4 (s, 1H), 8.33 (s, 2H), 8.23-8.15 (m, 3H), 7.40 (d, J = 8.4 Hz, |
| | | | | 2H), 7.33-7.27 (m, 1H), 7.23 (d, J = 8.4 Hz, 2H), 6.99-6.92 (m, |
| | | | | 2H), 6.04 (q, J = 6.4 Hz, 1H), 5.88 (br d, J = 8.0 Hz, 1H), 5.05 (br |
| | | | | dd, J = 4.4, 12.8 Hz, 1H), 4.40-4.24 (m, 5H), 4.18-4.11 (m, 2H), |
| | | | | 3.83 (br dd, J = 5.6, 10.8 Hz, 1H), 3.76 (br d, J = 8.0 Hz, 1H), 3.68- |
| | | | | 3.58 (m, 3H), 3.55 (br t, J = 4.4 Hz, 4H), 3.49-3.39 (m, 5H), 3.02- |
| | | | | 2.78 (m, 5H), 2.36-2.25 (m, 7H), 2.16-1.99 (m, 4H), 1.98-1.91 |
| | | | | (m, 1H), 1.71 (br d, J = 18.4 Hz, 2H), 1.87-1.65 (m, 1H), 1.62- |
| | | | | 1.52 (m, 6H), 1.47-1.37 (m, 1H), 0.97 (d, J = 5.6 Hz, 6H) |
| I-362 | JD | SL | 936.5 | 0.92 (s, 9 H) 1.34 (br d, J = 7.2 Hz, 4 H) 1.75 (ddd, J = 12.8, 8.4, 4.4 |
| | | | | Hz, 1 H) 1.81-1.87 (m, 2 H) 1.91-2.11 (m, 7 H) 2.16-2.42 (m, 6 |
| | | | | H) 2.85 (br t, J = 12.0 Hz, 3 H) 3.01-3.23 (m, 8 H) 3.60 (br s, 2 H) |
| | | | | 4.05 (br d, J = 12.0 Hz, 1 H) 4.13 (s, 1 H) 4.27 (br s, 1 H) 4.40 (t, |
| | | | | J = 8.0 Hz, 1 H) 4.50 (d, J = 9.2 Hz, 1 H) 4.66 (br d, J = 12.4 Hz, 2 H) |
| | | | | 4.88 (quin, J = 7.2 Hz, 1 H) 5.11 (br s, 1 H) 6.82-6.87 (m, 2 H) 7.19- |
| | | | | 7.23 (m, 2 H) 7.27-7.35 (m, 3 H) 7.42 (d, J = 8.4 Hz, 2 H) 7.62 |
| | | | | (br d, J = 9.2 Hz, 1 H) 7.92 (dd, J = 8.4, 1.31 Hz, 1 H) 8.24 (s, 2 H) |
| | | | | 8.36 (d, J = 7.6 Hz, 1 H) |
| I-363 | JD | SM | 936.5 | 0.93 (s, 9 H) 1.33 (br d, J = 7.2 Hz, 4 H) 1.75 (ddd, J = 12.8, 8.4, 4.8 |
| | | | | Hz, 1H) 1.81-1.87 (m, 2 H) 1.92-2.01 (m, 4 H) 2.05-2.22 (m, 5 |
| | | | | H) 2.42 (br s, 4 H) 2.85 (br t, J = 12.0 Hz, 3 H) 3.03-3.21 (m, 8 H) |
| | | | | 3.60 (br s, 2 H) 4.05 (br d, J = 12.0 Hz, 1 H) 4.12 (s, 1 H) 4.27 (br s, |
| | | | | 1 H) 4.40 (br t, J = 8.0 Hz, 1 H) 4.50 (d, J = 9.2 Hz, 1 H) 4.66 (br d, |
| | | | | J = 12.8 Hz, 2 H) 4.83-4.91 (m, 1 H) 5.11 (br s, 1 H) 6.82-6.87 (m, |
| | | | | 2 H) 7.19-7.23 (m, 2 H) 7.27-7.35 (m, 3 H) 7.42 (d, J = 8.4 Hz, 2 |
| | | | | H) 7.62 (br d, J = 9.2 Hz, 1 H) 7.92 (br d, J = 7.2 Hz, 1 H) 8.22-8.25 |
| | | | | (m, 2 H) 8.36 (d, J = 7.6 Hz, 1 H) |
| I-393 | XO | KC | 996.8 | 12.62-12.36 (m, 1H), 8.71 (s, 1H), 8.63 (t, J = 6.0 Hz, 1H), 8.44 |
| | | | | (s, 1H), 8.36 (s, 1H), 8.33 (s, 2H), 8.24-8.18 (m, 1H), 7.84 (d, J = |
| | | | | 12.4 Hz, 1H), 7.58-7.48 (m, 2H), 7.33-7.27 (m, 1H), 6.99-6.93 |
| | | | | (m, 2H), 6.09-5.99 (m, 1H), 5.61 (d, J = 9.2 Hz, 1H), 5.11-5.01 |
| | | | | (m, 1H), 4.69 (s, 1H), 4.40 (t, J = 8.0 Hz, 1H), 4.38-4.28 (m, 4H), |
| | | | | 3.90-3.81 (m, 2H), 3.77-3.70 (m, 2H), 3.63 (s, 2H), 3.31-3.19 |
| | | | | (m, 2H), 2.98-2.89 (m, 2H), 2.89-2.76 (m, 2H), 2.57-2.52 (m, |
| | | | | 1H), 2.41-2.32 (m, 1H), 2.27-2.23 (m, 1H), 2.09-2.02 (m, 2H), |
| | | | | 1.99-1.84 (m, 4H), 1.81 (d, J = 2.4 Hz, 3H), 1.78 (d, J = 2.4 Hz, |
| | | | | 3H), 1.76-1.66 (m, 3H), 1.64-1.56 (m, 2H), 1.54 (d, J = 6.8 Hz, |
| | | | | 3H), 0.94-0.89 (m, 9H) |
| I-394 | XP | KC | 995.9 | 12.46 (s, 1H), 8.72 (s, 1H), 8.54 (t, J = 6.0 Hz, 1H), 8.44 (s, 1H), |
| | | | | 8.34 (s, 2H), 8.27 (s, 1H), 8.24-8.19 (m, 1H), 7.72-7.65 (m, 1H), |
| | | | | 7.62-7.56 (m, 1H), 7.53-7.45 (m, 1H), 7.33-7.27 (m, 1H), 7.02- |
| | | | | 6.91 (m, 2H), 6.09-5.99 (m, 1H), 5.66 (d, J = 9.2 Hz, 1H), 5.10- |
| | | | | 5.01 (m, 1H), 4.75-4.64 (m, 2H), 4.40 (t, J = 8.0 Hz, 1H), 4.36- |
| | | | | 4.29 (m, 2H), 4.29-4.27 (m, 1H), 3.91-3.81 (m, 2H), 3.78-3.71 |
| | | | | (m, 2H), 3.64 (s, 2H), 3.48 (s, 1H), 3.33-3.23 (m, 2H), 2.98-2.90 |
| | | | | (m, 2H), 2.85 (d, J = 10.8 Hz, 2H), 2.58-2.55 (m, 1H), 2.43-2.35 |
| | | | | (m, 1H), 2.26 (d, J = 8.8 Hz, 2H), 2.08-1.98 (m, 2H), 1.95-1.86 |
| | | | | (m, 3H), 1.79 (d, J = 7.2 Hz, 3H), 1.75 (d, J = 7.2 Hz, 3H), 1.71 (d, |
| | | | | J = 11.2 Hz, 2H), 1.64-1.57 (m, 2H), 1.54 (d, J = 6.8 Hz, 3H), 0.91 |
| | | | | (s, 9H) |
| I-402 | YG | KC | 956.1 | 0.90 (s, 9 H) 1.50-1.62 (m, 5 H) 1.67-1.77 (m, 4 H) 1.85-1.94 |
| | | | | (m, 3 H) 1.98-2.05 (m, 1 H) 2.26 (d, J = 9.26 Hz, 2 H) 2.37 (d, |
| | | | | J = 2.88 Hz, 1 H) 2.85 (d, J = 11.13 Hz, 2 H) 2.89-2.98 (m, 2 H) 3.56 |
| | | | | (s, 1 H) 3.65 (s, 2 H) 3.69-3.79 (m, 3 H) 3.82-3.90 (m, 2 H) 4.12 |
| | | | | (dd, J = 16.70, 4.94 Hz, 1 H) 4.28-4.39 (m, 3 H) 4.47 (dd, J = 16.70, |
| | | | | 6.69 Hz, 1 H) 4.67-4.78 (m, 1 H) 4.99-5.22 (m, 1 H) 5.05 (dd, |
| | | | | J = 13.45, 5.07 Hz, 1 H) 5.64 (d, J = 9.51 Hz, 1 H) 6.04 (q, J = 6.71 Hz, |
| | | | | 1 H) 6.90-7.03 (m, 2 H) 7.17 (d, J = 8.63 Hz, 2 H) 7.29 (t, J = 7.25 |
| | | | | Hz, 1 H) 8.18-8.23 (m, 1 H) 8.33 (s, 2 H) 8.64-8.74 (m, 2 H) |
| | | | | 12.40-12.48 (m, 1 H) 14.19-14.44 (m, 1 H) |
| I-417 | ZC | KC | 980.3 | 0.89-0.97 (m, 9 H) 1.50-1.62 (m, 5 H) 1.66-1.76 (m, 4 H) 1.80- |
| | | | | 2.04 (m, 4 H) 1.83-1.95 (m, 1 H) 2.20-2.29 (m, 2 H) 2.32-2.43 |
| | | | | (m, 2 H) 2.79-2.87 (m, 2 H) 2.89-2.97 (m, 2 H) 3.58-3.66 (m, 2 |
| | | | | H) 3.69-3.77 (m, 2 H) 3.79-3.89 (m, 5 H) 3.97 (s, 3 H) 4.24- |
| | | | | 4.42 (m, 5 H) 4.98-5.14 (m, 2 H) 5.56 (d, J = 9.38 Hz, 1 H) 6.03 |
| | | | | (m, J = 6.75 Hz, 1 H) 6.91-7.01 (m, 2 H) 7.24-7.34 (m, 2 H) 7.35- |
| | | | | 7.40 (m, 1 H) 7.47 (s, 1 H) 8.20 (dd, J = 8.38, 1.50 Hz, 1 H) 8.33 |
| | | | | (s, 2 H) 8.41-8.47 (m, 1 H) 8.71 (s, 1 H) 12.44 (s, 1 H) 14.32 (s, 1 H) |
| I-420 | ZB | KC | 965.5 | 0.93 (s, 9 H) 1.54 (d, J = 6.4 Hz, 3 H) 1.57-1.65 (m, 2 H) 1.66-2.04 |
| | | | | (m, 9 H) 2.20-2.42 (m, 4 H) 2.82-2.96 (m, 4 H) 3.10 (s, 3 H) 3.41 |
| | | | | (br s, 1 H) 3.48 (s, 2 H) 3.63 (br s, 2 H) 3.74 (q, J = 8.4 Hz, 2 H) 3.81- |
| | | | | 3.89 (m, 2 H) 4.12-4.18 (m, 1 H) 4.27-4.35 (m, 3 H) 4.38 (t, |
| | | | | J = 8.0 Hz, 1 H) 5.05 (br dd, J = 13.2 Hz, 1H)5.11 (d, J = 3.6 Hz, 1 H) |
| | | | | 5.60 (d, J = 9.2 Hz, 1 H) 6.04 (q, J = 6.4 Hz, 1 H) 6.87 (d, J = 7.6 Hz, |
| | | | | 1 H) 6.94-6.99 (m, 2 H) 7.16-7.22 (m, 2 H) 7.27-7.32 (m, 1 H) |
| | | | | 8.19-8.23 (m, 1 H) 8.34 (s, 2 H) 8.44 (t, J = 6.0 Hz, 1 H) 8.71 (s, 1 |
| | | | | H) 12.44 (s, 1 H) 14.33 (br s, 1 H) |
| I-421^{c} | ZA | KC | 483.4 | 14.62-13.95 (m, 1H), 12.84-12.08 (m, 1H), 8.71 (s, 1H), 8.54- |
| | | | | 8.30 (m, 4H), 8.21 (d, J = 7.6 Hz, 1H), 7.36-7.23 (m, 1H), 7.14 (d, |
| | | | | J = 7.6 Hz, 1H), 7.02-6.88 (m, 4H), 6.08-5.99 (m, 1H), 5.48 (d, J = |
| | | | | 9.2 Hz, 1H), 5.10-5.00 (m, 1H), 4.48-4.27 (m, 4H), 4.14 (dd, J = |
| | | | | 5.6, 15.6 Hz, 1H), 3.85 (s, 1H), 3.73 (s, 2H), 3.62 (s, 3H), 3.50 (s, |
| | | | | 3H), 3.14-3.11 (m, 3H), 2.97-2.81 (m, 4H), 2.58-2.52 (m, 1H), |
| | | | | 2.38 (dd, J = 1.6, 8.8 Hz, 1H), 2.30-2.19 (m, 2H), 2.11-1.81 (m, |
| | | | | 6H), 1.78-1.68 (m, 3H), 1.58 (d, J = 12.4 Hz, 2H), 1.54 (d, J = 6.4 |
| | | | | Hz, 3H), 0.91 (s, 9H) |

^{a}The reaction was run at rt anywhere from 2-16 hr. Separation of the stereoisomers by prep-HPLC or SFC under a variety of solvent conditions. The absolute stereochemistry of the diastereomers was assigned arbitrarily. Racemic compounds were purified by prep-HPLC or reverse phase flash chromatography under a variety of conditions.
^{b}Diastereomer mixture at CH—iPr.
^{c}Cis/trans mixture.
^{d}Chiral center on spirocycle arbitrarily assigned.
^{e}LC-MS (ESI⁺) m/z = (M/2 + H)⁺.
^{f}The product of the coupling was further deprotected with CsF in DMSO at 50-55° C. for 1 hr. The final product was purified by HPLC.
^{g}The product of the coupling was further deprotected with LiOH—H₂O. in THF, H₂O, and MeOH at 25° C. for 0.5 hr. The final product was purified by reverse phase flash chromatography.
^{h}The ¹H NMR reported used CDCl₃ as the solvent.
ⁱLC-MS (ESI⁺) m/z = (M/2 − 2)⁺.

TABLE 3B
Compounds synthesized via Method 1 using the corresponding amines and acids for the coupling.

| | | | LCMS (ESI+) | |
|---|---|---|---|---|
| I-#^{a} | Amine | Acid | m/z (M + H)⁺ | ¹H NMR (400 MHz, DMSO-d6) |
| I-87 | GL | FA | 959.6 | .74 (s, 2H), 8.56 (br t, J = 6.0 Hz, 1H), 7.95 (d, J = 8.0 Hz, 1H), 7.65 |
| | | | | (br d, J = 9.2 Hz, 1H), 7.57 (d, J = 8.4 Hz, 2H), 7.45 (d, J = 2.0 Hz, |
| | | | | 1H), 7.42 (s, 4H), 7.33 (s, 1H), 7.29 (d, J = 8.4 Hz, 2H), 7.24-7.19 |
| | | | | (m, 1H), 6.90-6.83 (m, 2H), 6.35 (d, J = 2.0 Hz, 1H), 5.14 (d, J = |
| | | | | 3.6 Hz, 1H), 4.80 (br dd, J = 4.4, 8.4 Hz, 2H), 4.59 (d, J = 9.6 Hz, |
| | | | | 1H), 4.47-4.39 (m, 2H), 4.36 (br d, J = 1.2 Hz, 1H), 4.28-4.21 (m, |
| | | | | 2H), 3.83 (s, 3H), 3.68 (br s, 1H), 3.30 (br s, 3H), 3.05-3.00 (m, |
| | | | | 1H), 2.90-2.81 (m, 1H), 2.08-2.02 (m, 2H), 1.94-1.89 (m, 2H), |
| | | | | 1.82-1.77 (m, 1H), 1.69-1.59 (m, 4H), 1.57-1.49 (m, 2H), 1.23 |
| | | | | (s, 2H), 1.18-1.13 (m, 1H), 0.97-0.93 (m, 9H), 0.87-0.83 (m, |
| | | | | 1H), 0.81-0.77 (m, 1H) |
| I-88 | GL | FA | 959.3 | 14.72 (s, 1H), 8.75 (s, 2H), 8.57 (t, J = 6.2 Hz, 1H), 7.96 (d, J = 8.0 |
| | | | | Hz, 1H), 7.77 (br d, J = 9.6 Hz, 1H), 7.58 (d, J = 8.0 Hz, 2H), 7.46 |
| | | | | (d, J = 2.0 Hz, 1H), 7.43 (s, 3H), 7.43 (br s, 1H), 7.33 (d, J = 5.2 |
| | | | | Hz, 2H), 7.31 (s, 1H), 7.24-7.20 (m, 1H), 6.90-6.85 (m, 2H), 6.37 |
| | | | | (d, J = 2.0 Hz, 1H), 5.14 (d, J = 3.6 Hz, 1H), 4.83-4.78 (m, 2H), |
| | | | | 4.55 (d, J = 9.2 Hz, 1H), 4.47-4.42 (m, 2H), 4.36 (br d, J = 2.0 Hz, |
| | | | | 1H), 4.28-4.23 (m, 2H), 3.84 (s, 2H), 3.83 (s, 1H), 3.69-3.63 (m, |
| | | | | 3H), 3.30 (s, 3H), 3.24 (br d, J = 2.4 Hz, 1H), 3.05-2.98 (m, 1H), |
| | | | | 2.86 (t, J = 12.4 Hz, 1H), 2.07-2.03 (m, 1H), 1.94-1.83 (m, 5H), |
| | | | | 1.78 (br d, J = 12.4 Hz, 1H), 1.58-1.46 (m, 4H), 1.24 (s, 1H), 0.96 |
| | | | | (s, 9H) |
| I-131^{c} | KV | KR | 980.6 | 8.52 (t, J = 6.0 Hz, 1H), 8.35 (s, 2H), 7.94 (d, J = 8.0 Hz, 1H), 7.76 |
| | | | | (d, J = 9.6 Hz, 1H), 7.38 (d, J = 8.0 Hz, 3H), 7.33-7.28 (m, 3H), |
| | | | | 7.24-7.17 (m, 1H), 6.90-6.82 (m, 2H), 5.10 (s, 1H), 4.72 (dd, J = |
| | | | | 4.8, 8.0 Hz, 2H), 4.54 (d, J = 9.6 Hz, 1H), 4.43-4.29 (m, 4H), 4.25- |
| | | | | 4.14 (m, 2H), 4.12 (s, 1H), 3.61 (dt, J = 3.6, 7.6 Hz, 4H), 3.18- |
| | | | | 3.07 (m, 2H), 3.02-2.84 (m, 4H), 2.75 (dd, J = 10.8, 12.8 Hz, 1H), |
| | | | | 2.63 (d, J = 8.0 Hz, 1H), 2.56-2.53 (m, 1H), 2.42-2.35 (m, 1H), |
| | | | | 2.30-2.23 (m, 1H), 1.99 (d, J = 7.6 Hz, 2H), 1.91-1.82 (m, 1H), |
| | | | | 1.71 (d, J = 10.4 Hz, 6H), 1.66-1.58 (m, 2H), 1.57-1.47 (m, 5H), |
| | | | | 1.46-1.38 (m, 3H), 1.29 (d, J = 8.8 Hz, 2H), 1.26-1.19 (m, 1H), |
| | | | | 1.11-1.03 (m, 1H), 1.00-0.94 (m, 3H), 0.92 (s, 3H) |
| I-132^{b, d} | IP | EM | 977.8 | 14.44.13.96 (m, 1H), 9.79-9.41 (m, 1H), 8.54-8.44 (m, 2H), 8.35 |
| | | | | (s, 2H), 8.22-8.10 (m, 2H), 8.05 (d, J = 8.8 Hz, 1H), 7.52-7.46 |
| | | | | (m, 1H), 7.41 (d, J = 8.0 Hz, 3H), 7.27 (d, J = 8.0 Hz, 2H), 7.19 (s, |
| | | | | 1H), 7.07-6.98 (m, 2H), 4.73 (d, J = 12.0 Hz, 1H), 4.66-4.55 (m, |
| | | | | 1H), 4.47-4.41 (m, 1H), 4.39-4.33 (m, 2H), 4.31-4.23 (m, 3H), |
| | | | | 4.16 (s, 1H), 3.71 (d, J = 6.0 Hz, 2H), 3.30 (s, 2H), 3.07-2.90 (m, |
| | | | | 3H), 2.87-2.72 (m, 6H), 2.70-2.63 (m, 2H), 2.25-2.17 (m, 2H), |
| | | | | 2.13-1.89 (m, 8H), 1.88-1.80 (m, 4H), 1.72 (d, J = 10.8 Hz, 2H), |
| | | | | 1.61 (dd, J = 3.2, 7.6 Hz, 2H), 1.53-1.17 (m, 8H) |
| I-192 | LN | LO | 941.9 | 12.42-12.15 (m, 1H), 8.55-8.50 (m, 2H), 8.18 (s, 1H), 8.11 (dd, J = |
| | | | | 1.2, 8.4 Hz, 1H), 7.39-7.35 (m, 2H), 7.32-7.26 (m, 3H), 6.98- |
| | | | | 6.92 (m, 2H), 5.83 (d, J = 8.8 Hz, 1H), 4.44-4.31 (m, 7H), 4.19 |
| | | | | (dd, J = 5.6, 16.0 Hz, 1H), 4.12 (s, 1H), 4.02-3.90 (m, 4H), 3.71- |
| | | | | 3.62 (m, 4H), 3.45 (d, J = 5.6 Hz, 1H), 3.11-3.05 (m, 2H), 2.98- |
| | | | | 2.87 (m, 4H), 2.84-2.73 (m, 4H), 2.70 (s, 1H), 2.54 (s, 1H), 2.52 |
| | | | | (d, J = 2.0 Hz, 2H), 2.24 (t, J = 10.4 Hz, 2H), 2.04-1.98 (m, 1H), |
| | | | | 1.88 (dt, J = 4.4, 8.4 Hz, 2H), 1.81 (s, 2H), 1.56-1.46 (m, 4H), 1.38 |
| | | | | (d, J = 6.4 Hz, 3H), 1.26-1.18 (m, 1H), 0.94 (s, 8H) |
| I-233^{c} | JD | OH | 965.4 | 15.21-14.18 (m, 1H), 8.36 (br d, J = 8.0 Hz, 1H), 8.24 (s, 2H), 7.95 |
| | | | | (br d, J = 7.6 Hz, 1H), 7.64 (br d, J = 9.6 Hz, 1H), 7.44-7.37 (m, |
| | | | | 3H), 7.34-7.27 (m, 3H), 7.25-7.19 (m, 1H), 6.90-6.83 (m, 2H), |
| | | | | 5.10 (br s, 1H), 4.94-4.82 (m, 1H), 4.63 (br d, J = 12.0 Hz, 2H), |
| | | | | 4.48 (br d, J = 8.0 Hz, 1H), 4.40 (br t, J = 7.6 Hz, 1H), 4.30-4.25 |
| | | | | (m, 1H), 4.23-4.17 (m, lH), 4.13 (s, 1H), 3.64-3.56 (m, 3H), 3.06- |
| | | | | 2.95 (m, 6H), 2.68 (br s, 3H), 2.38-2.34 (m, 4H), 2.31-2.28 (m, |
| | | | | 1H), 2.02-1.96 (m, 2H), 1.83-1.71 (m, 3H), 1.58-1.42 (m, 7H), |
| | | | | 1.36-1.25 (m, 7H), 0.92 (s, 9H) |
| I-234^{c} | JD | OE | 965.4 | 14.17 (br dd, J = 3.2, 4.4 Hz, 1H), 10.53 (br dd, J = 1.8, 6.0 Hz, 1H), |
| | | | | 9.62-9.36 (m, 1H), 8.36-8.30 (m, 3H), 8.17 (br s, 1H), 7.68 (br d, |
| | | | | J = 8.4 Hz, 1H), 7.47 (br d, J = 8.0 Hz, 1H), 7.44-7.39 (m, 3H), |
| | | | | 7.28 (br d, J = 8.0 Hz, 2H), 7.20-7.17 (m, 1H), 7.08-6.99 (m, 2H), |
| | | | | 4.87 (br t, J = 7.2 Hz, 1H), 4.65 (br d, J = 12.4 Hz, 1H), 4.58-4.53 |
| | | | | (m, 1H), 4.50-4.45 (m, 1H), 4.38 (br t, J = 8.0 Hz, 1H), 4.30-4.21 |
| | | | | (m, 2H), 4.13 (s, 1H), 3.70 (br s, 4H), 3.62-3.53 (m, 3H), 3.34- |
| | | | | 3.27 (m, 3H), 3.23 (br d, J = 0.8 Hz, 1H), 3.01-2.92 (m, 3H), 2.91- |
| | | | | 2.87 (m, 2H), 2.70-2.65 (m, 1H), 2.54 (br s, 2H), 2.33 (br s, 1H), |
| | | | | 2.23-2.17 (m, 1H), 2.03-1.96 (m, 2H), 1.89-1.82 (m, 1H), 1.77- |
| | | | | 1.61 (m, 4H), 1.53-1.47 (m, 1H), 1.33 (br d, J = 6.8 Hz, 6H), 0.92 |
| | | | | (s, 9H) |
| I-244 | HH | PF | 887.5 | 8.55 (t, J = 6 Hz, 1H), 7.95-7.87 (m, 1H), 7.82 (d, J = 9.2 Hz, 1H), |
| | | | | 7.38-7.32 (m, 5H), 7.23-7.18 (m, 2H), 6.88-6.81 (m, 2H), 5.23- |
| | | | | 5.04 (m, 1H), 4.53 (d, J = 9.6 Hz, 1H), 4.45-4.37 (m, 2H), 4.35 |
| | | | | (s, 1H), 4.19 (d, J = 5.6 Hz, 1H), 4.16-4.13 (m, 1H), 4.05 (d, J = |
| | | | | 12 Hz, 1H), 3.69-3.61 (m, 2H), 3.52-3.45 (m, 2H), 3.21-3.16 |
| | | | | (m, 2H), 3.13 (s, 2H), 3.03 (s, 2H), 2.89 (d, J = 9.6 Hz, 2H), 2.81 (s, |
| | | | | 1H), 2.72 (s, 1H), 2.24-2.12 (m, 6H), 2.01 (d, J = 9.2 Hz, 1H), 1.94- |
| | | | | 1.85 (m, 6H), 1.63-1.55 (m, 4H), 1.16-1.06 (m, 3H), 1.01 (d, J = |
| | | | | 6.4 Hz, 3H), 0.95-0.90 (m, 9H) |
| I-248^{c} | PA | KR | 980.4 | 8.35 (s, 2H), 8.17-8.07 (m, 1H), 7.94 (br d, J = 8.0 Hz, 1H), 7.88 |
| | | | | (br d, J = 7.6 Hz, 1H), 7.44-7.36 (m, 3H), 7.31 (s, 1H), 7.27-7.18 |
| | | | | (m, 3H), 6.90-6.81 (m, 2H), 5.14 (br d, J = 0.8 Hz, 1H), 4.72 (br |
| | | | | d, J = 13.2 Hz, 2H), 4.42-4.29 (m, 4H), 4.29-4.16 (m, 3H), 4.13 |
| | | | | (s, 1H), 3.79 (br dd, J = 5.2, 10.4 Hz, 1H), 3.66-3.57 (m, 2H), 3.46 |
| | | | | (br t, J = 6.8 Hz, 3H), 3.19-3.11 (m, 2H), 3.08-2.97 (m, 3H), 2.96 |
| | | | | (br d, J = 2.8 Hz, 1H), 2.80-2.73 (m, 1H), 2.54 (s, 1H), 2.22 (br s, |
| | | | | 1H), 2.14-1.98 (m, 3H), 1.98-1.92 (m, 1H), 1.87 (br d, J = 10.0 |
| | | | | Hz, 1H), 1.85-1.75 (m, 3H), 1.67 (br d, J = 13.6 Hz, 3H), 1.63- |
| | | | | 1.57 (m, 2H), 1.55 (br d, J = 6.8 Hz, 1H), 1.50 (br d, J = 7.2 Hz, |
| | | | | 4H), 1.32 (br s, 1H), 1.24 (br s, 2H), 1.22-1.14 (m, 2H), 0.97 (s, 6H) |

^{a}The reaction was run anywhere from 0-25° C. for 10 min to 16 hrs. The coupled products were purified by prep-HPLC or SFC under variety of conditions. Absolute stereochemistry of the separated diastereomers was assigned arbitrarily for all compounds.
^{b}Cis-trans mixture at spirocycle.
^{c}Chiral center on spirocycle arbitrarily assigned.
^{d}No SFC separation, after coupling, HCl/dioxane at rt in DCM at rt was used to deprotect the amine. The final compound was purified by prep-HPLC.

### Example 2 (Method 2): Syntheses of 2S,4R)-1-((S)-2-(1-fluorocyclopropane-1-carboxamido)-3,3-dimethylbutanoyl)-4-hydroxy-N-(2-(((1R,4S)-4-(4-(2-((R)-3-(2-hydroxyphenyl)-5-methyl-5,7,8,9-tetrahydro-6H-pyrido[3′,4′:4,5]pyrrolo[2,3-c]pyridazin-6-yl)pyrimidin-5-yl)piperidin-1-yl)cyclohexyl)oxy)-4-(4-methylthiazol-5-yl)benzyl)pyrrolidine-2-carboxamide (I-30) and (2S,4R)-1-((S)-2-(1-fluorocyclopropane-1-carboxamido)-3,3-dimethylbutanoyl)-4-hydroxy-N-(2-(((1S,4R)-4-(4-(2-((R)-3-(2-hydroxyphenyl)-5-methyl-5,7,8,9-tetrahydro-6H-pyrido[3′,4′:4,5]pyrrolo[2,3-c]pyridazin-6-yl)pyrimidin-5-yl)piperidin-1-yl)cyclohexyl)oxy)-4-(4-methylthiazol-5-yl)benzyl)pyrrolidine-2-carboxamide (I-31)

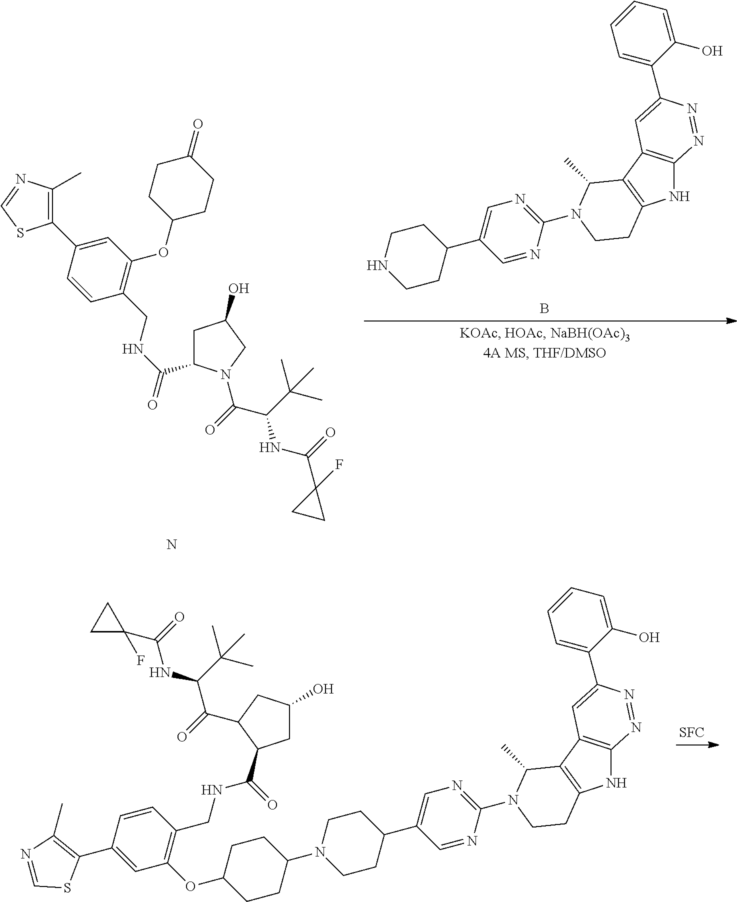

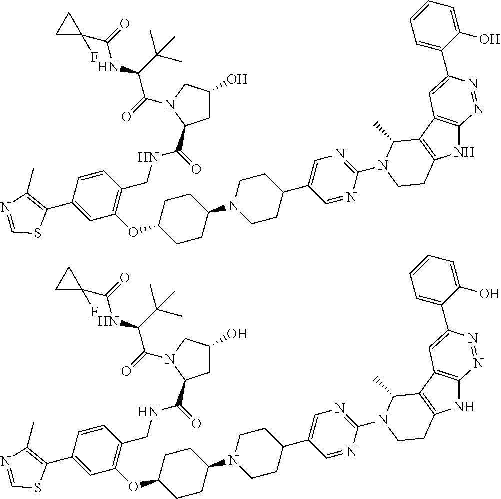 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@H](N3CCC(c4cnc(N5CCc6[nH]c7nnc(-c8ccccc8O)cc7c6[C@H]5C)nc4)CC3)CC2)c1 ; Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@@H](N3CCC(c4cnc(N5CCc6[nH]c7nnc(-c8ccccc8O)cc7c6[C@H]5C)nc4)CC3)CC2)c1]

Step 1-(2S,4R)-1-((S)-2-(1-fluorocyclopropanecarboxamido)-3,3-dimethylbutanoyl)-4-hydroxy-N-(2-((4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)oxy)-4-(4-methylthiazol-5-yl)benzyl)pyrrolidine-2-carboxamide. To a solution of (R)-2-(5-methyl-6-(5-(piperidin-4-yl)pyrimidin-2-yl)-6,7,8,9-tetrahydro-5H-pyrido[3′,4′:4,5]pyrrolo[2,3-c]pyridazin-3-yl)phenol (38.0 mg, 79.5 umol, Intermediate B) in DMSO (1 mL) and THF (1 mL) was added KOAc (23.4 mg, 238 umol), 4 Å molecular sieves (30 mg), HOAc (14.0 mg 238 umol) and (2S,4R)-1-((S)-2-(1-fluorocyclopropanecarboxamido)-3,3-dimethylbutanoyl)-4-hydroxy-N-(4-(4-methylthiazol-5-yl)-2-((4-oxocyclohexyl)oxy)benzyl)pyrrolidine-2-carboxamide (50 mg, 79.5 umol Intermediate N). The mixture was stirred at 0° C. for 0.5 hours, and then NaBH(OAc)₃ (50.6 mg, 238 umol) was added. The mixture was stirred at 30° C. for 11.5 hours. On completion, the reaction mixture was quenched with H₂O 1 mL at 0° C., and then filtered and concentrated under reduced pressure to give a residue. The residue was purified by prep-HPLC (FA condition) to give the title compound (50 mg, 34.9 umol, 59% yield) as a white solid. LC-MS (ESI⁺) m/z 1054.3 (M+H)⁺.

Step 2-(2S,4R)-1-((S)-2-(1-fluorocyclopropane-1-carboxamido)-3,3-dimethylbutanoyl)-4-hydroxy-N-(2-(((1R,4S)-4-(4-(2-((R)-3-(2-hydroxyphenyl)-5-methyl-5,7,8,9-tetrahydro-6H-pyrido[3′,4′:4,5]pyrrolo[2,3-c]pyridazin-6-yl)pyrimidin-5-yl)piperidin-1-yl)cyclohexyl)oxy)-4-(4-methylthiazol-5-yl)benzyl)pyrrolidine-2-carboxamide and (2S,4R)-1-((S)-2-(1-fluorocyclopropane-1-carboxamido)-3,3-dimethylbutanoyl)-4-hydroxy-N-(2-(((1S,4R)-4-(4-(2-((R)-3-(2-hydroxyphenyl)-5-methyl-5,7,8,9-tetrahydro-6H-pyrido[3′,4′:4,5]pyrrolo[2,3-c]pyridazin-6-yl)pyrimidin-5-yl)piperidin-1-yl)cyclohexyl)oxy)-4-(4-methylthiazol-5-yl)benzyl)pyrrolidine-2-carboxamide. The mixture of (2S,4R)-1-((S)-2-(1-fluorocyclopropanecarboxamido)-3,3-dimethylbutanoyl)-4-hydroxy-N-(2-((4-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)cyclohexyl)oxy)-4-(4-methylthiazol-5-yl)benzyl)pyrrolidine-2-carboxamide was separated by SFC (column: DAICEL CHIRALCEL OD (250 mm*50 mm, 10 um); mobile phase: [0.1% NH₃H2O MEOH]; B %: 55%-55%, 5; 40 min) to give (2S,4R)-1-((S)-2-(1-fluorocyclopropane-1-carboxamido)-3,3-dimethylbutanoyl)-4-hydroxy-N-(2-(((1R,4S)-4-(4-(2-((R)-3-(2-hydroxyphenyl)-5-methyl-5,7,8,9-tetrahydro-6H-pyrido[3′,4′:4,5]pyrrolo[2,3-c]pyridazin-6-yl)pyrimidin-5-yl)piperidin-1-yl)cyclohexyl)oxy)-4-(4-methylthiazol-5-yl)benzyl)pyrrolidine-2-carboxamide (20 mg, 18.8 umol) (¹H NMR (400 MHz, DMSO-d₆) δ=14.40 (s, 1H), 12.48 (s, 1H), 9.04 (s, 1H), 8.78 (s, 1H), 8.56 (t, J=5.6 Hz, 1H), 8.40 (s, 2H), 8.29-8.25 (m, 1H), 7.47 (d, J=7.6 Hz, 1H), 7.39-7.32 (m, 2H), 7.08 (s, 1H), 7.05-6.97 (m, 3H), 6.14-6.07 (m, 1H), 5.26-5.19 (m, 1H), 5.15-5.08 (m, 1H), 4.78 (s, 1H), 4.68-4.62 (m, 1H), 4.58 (t, J=8.4 Hz, 1H), 4.46-4.28 (m, 3H), 3.74-3.62 (m, 2H), 3.54-3.46 (m, 2H), 3.07-2.95 (m, 4H), 2.59 (s, 1H), 2.51 (s, 3H), 2.48-2.41 (m, 2H), 2.37-2.28 (m, 2H), 2.19-2.14 (m, 1H), 2.12-2.04 (m, 2H), 2.04-1.96 (m, 1H), 1.82-1.76 (m, 2H), 1.73-1.64 (m, 7H), 1.60 (d, J=6.6 Hz, 3H), 1.47-1.37 (m, 2H), 1.30-1.25 (m, 2H), 0.98 (s, 9H); LC-MS (ESI⁺) m/z 1054.3 (M+H)⁺) as white solid and 2S,4R)-1-((S)-2-(1-fluorocyclopropane-1-carboxamido)-3,3-dimethylbutanoyl)-4-hydroxy-N-(2-(((1S,4R)-4-(4-(2-((R)-3-(2-hydroxyphenyl)-5-methyl-5,7,8,9-tetrahydro-6H-pyrido[3′,4′:4,5]pyrrolo[2,3-c]pyridazin-6-yl)pyrimidin-5-yl)piperidin-1-yl)cyclohexyl)oxy)-4-(4-methylthiazol-5-yl)benzyl)pyrrolidine-2-carboxamide (13 mg, 12.1 umol) (¹H NMR (400 MHz, DMSO-d₆) δ=14.34 (s, 1H), 12.52-12.35 (m, 1H), 8.99 (s, 1H), 8.72 (s, 1H), 8.46 (t, J=6.0 Hz, 1H), 8.34 (s, 2H), 8.22 (d, J=7.2 Hz, 1H), 7.40 (d, J=7.6 Hz, 1H), 7.33-7.26 (m, 2H), 7.07 (s, 1H), 6.99-6.92 (m, 3H), 6.08-6.01 (m, 1H), 5.23-5.11 (m, 1H), 5.10-5.01 (m, 1H), 4.62-4.48 (m, 2H), 4.43-4.33 (m, 2H), 4.30-4.15 (m, 2H), 3.68-3.57 (m, 2H), 3.45-3.41 (m, 2H), 3.08-2.94 (m, 4H), 2.53-2.53 (m, 1H), 2.46 (s, 3H), 2.42-2.36 (m, 2H), 2.30-2.22 (m, 2H), 2.16-2.05 (m, 3H), 2.00-1.90 (m, 1H), 1.82 (d, J=7.6 Hz, 2H), 1.76-1.69 (m, 2H), 1.67-1.58 (m, 2H), 1.55 (d, J=6.6 Hz, 3H), 1.47 (d, J=8.4 Hz, 3H), 1.41-1.34 (m, 2H), 1.24-1.21 (m, 2H), 1.02-0.94 (m, 9H); LC-MS (ESI⁺) m/z 1054.3 (M+H)⁺). Absolute stereochemistry of the diastereomers was assigned arbitrarily.

TABLE 4
Compounds synthesized via Method 2 using the corresponding amines
and aldehydes or ketones for the reductive amination.

| | | Aldehyde/ | LCMS (ESI+) | |
|---|---|---|---|---|
| I-#^{a} | Amine | Ketone | m/z (M + H)⁺ | ¹H NMR (400 MHz, DMSO-d6) |
| I-32 | M | AZ | 1003.2 | 8.99 (s, 1H), 8.50 (t, J = 6.0 Hz, 1H), 8.34 (s, 2H), 7.94 (d, J = |
| | | | | 8.0 Hz, 1H), 7.43-7.37 (m, 2H), 7.31 (s, 1H), 7.28 (dd, J = |
| | | | | 2.8, 9.6 Hz, 1H), 7.24-7.19 (m, 1H), 7.06 (d, J = 1.6 Hz, |
| | | | | 1H), 6.96 (dd, J = 1.2, 8.0 Hz, 1H), 6.89-6.83 (m, 2H), 5.17 |
| | | | | (d, J = 1.6 Hz, 1H), 4.76-4.68 (m, 2H), 4.59 (d, J = 9.2 Hz, |
| | | | | 1H), 4.52 (t, J = 8.4 Hz, 1H), 4.37-4.31 (m, 1H), 4.29 (d, J = |
| | | | | 6.0 Hz, 1H), 4.25-4.21 (m, 1H), 4.19 (t, J = 5.2 Hz, 3H), |
| | | | | 3.67-3.57 (m, 3H), 3.28-3.22 (m, 4H), 3.17-3.12 (m, 1H), |
| | | | | 3.10-3.05 (m, 2H), 2.95 (dt, J = 3.2, 12.4 Hz, 1H), 2.81- |
| | | | | 2.71 (m, 3H), 2.47 (s, 3H), 2.41-2.38 (m, 1H), 2.21-2.14 |
| | | | | (m, 2H), 2.12-2.06 (m, 1H), 1.92 (ddd, J = 4.4, 8.8, 12.8 Hz, |
| | | | | 1H), 1.74-1.62 (m, 4H), 1.41-1.32 (m, 2H), 1.21 (dd, J = |
| | | | | 3.2, 8.8 Hz, 2H), 0.95 (s, 9H) |
| I-33 | O | N | 1054.3 | 14.42 (s, 1H), 8.98 (s, 1H), 8.70 (s, 1H), 8.51 (t, J = 5.6 Hz, |
| | | | | 1H), 8.35 (s, 2H), 8.23-8.19 (m, 1H), 7.41 (d, J = 8.0 Hz, |
| | | | | 1H), 7.32-7.26 (m, 2H), 7.03 (s, 1H), 6.99-6.91 (m, 3H), |
| | | | | 6.09-6.02 (m, 1H), 5.18 (d, J = 2.4 Hz, 1H), 5.10-5.02 (m, |
| | | | | 1H), 4.73 (s, 1H), 4.63-4.49 (m, 2H), 4.40-4.22 (m, 3H), |
| | | | | 3.71-3.57 (m, 2H), 3.41 (s, 2H), 3.03-2.89 (m, 4H), 2.53- |
| | | | | 2.53 (m, 1H), 2.46 (s, 3H), 2.42-2.35 (m, 2H), 2.31-2.22 |
| | | | | (m, 2H), 2.14-2.07 (m, 1H), 2.06-2.00 (m, 2H), 1.99-1.90 |
| | | | | (m, 1H), 1.77-1.70 (m, 2H), 1.68-1.59 (m, 7H), 1.54 (d, J = |
| | | | | 6.4 Hz, 3H), 1.40-1.31 (m, 2H), 1.24-1.20 (m, 2H), 1.01- |
| | | | | 0.94 (m, 9H) |
| I-34 | O | N | 1054.3 | 14.39 (s, 1H), 9.04 (s, 1H), 8.77 (s, 1H), 8.51 (t, J = 5.6 Hz, |
| | | | | 1H), 8.39 (s, 2H), 8.29-8.24 (m, 1H), 7.45 (d, J = 7.8 Hz, |
| | | | | 1H), 7.38-7.30 (m, 2H), 7.12 (d, J = 1.3 Hz, 1H), 7.04-6.97 |
| | | | | (m, 3H), 6.13-6.06 (m, 1H), 5.25-5.18 (m, 1H), 5.15-5.07 |
| | | | | (m, 1H), 4.69-4.49 (m, 2H), 4.41 (s, 2H), 4.36-4.19 (m, |
| | | | | 2H), 3.73-3.61 (m, 2H), 3.54-3.45 (m, 2H), 3.07-2.90 (m, |
| | | | | 4H), 2.58 (d, J = 2.0 Hz, 1H), 2.51 (s, 3H), 2.48-2.40 (m, |
| | | | | 2H), 2.31 (t, J = 10.8 Hz, 2H), 2.22-2.11 (m, 3H), 2.04- |
| | | | | 1.95 (m, 1H), 1.87 (s, 2H), 1.78 (d, J = 12.4 Hz, 2H), 1.71- |
| | | | | 1.64 (m, 2H), 1.60 (d, J = 6.5 Hz, 3H), 1.57-1.49 (m, 3H), |
| | | | | 1.47-1.38 (m, 2H), 1.30-1.26 (m, 2H), 1.07-0.98 (m, 9H) |
| I-35^{c} | B | AD | 980.4 | 14.33 (s, 1H), 12.44 (s, 1H), 8.98 (d, J = 2.4 Hz, 1H), 8.71 (s, |
| | | | | 1H), 8.49 (td, J = 6.0, 19.2 Hz, 1H), 8.33 (d, J = 4.8 Hz, 2H), |
| | | | | 8.21 (dd, J = 1.2, 8.4 Hz, 1H), 7.45-7.35 (m, 3H), 7.34-7.26 |
| | | | | (m, 2H), 6.98-6.92 (m, 2H), 6.09-6.00 (m, 2H), 5.05 (dd, J = |
| | | | | 4.4, 13.6 Hz, 1H), 4.46-4.24 (m, 4H), 4.18-4.08 (m, 2H), |
| | | | | 3.79-3.74 (m, 1H), 3.60-3.53 (m, 1H), 3.48-3.39 (m, 3H), |
| | | | | 2.98-2.86 (m, 4H), 2.45 (s, 3H), 2.38-2.31 (m, 2H), 2.30- |
| | | | | 2.24 (m, 2H), 2.04 (dd, J = 3.6, 8.0 Hz, 1H), 1.97-1.86 (m, |
| | | | | 3H), 1.75-1.58 (m, 6H), 1.54 (d, J = 6.4 Hz, 3H), 1.51-1.44 |
| | | | | (m, 1H), 0.95 (t, J = 7.2 Hz, 3H), 0.81 (dd, J = 6.8, 13.6 Hz, |
| | | | | 3H), 0.70-0.52 (m, 1H) |
| I-36^{c} | FN | AL | 1035.4 | 14.33 (s, 1H), 8.98 (s, 1H), 8.71 (s, 1H), 8.54-8.44 (m, 1H), |
| | | | | 8.33 (d, J = 2.0 Hz, 2H), 8.21 (d, J = 7.2 Hz, 1H), 7.45-7.36 |
| | | | | (m, 3H), 7.30 (dd, J = 8.4, 16.8 Hz, 2H), 6.99-6.92 (m, 2H), |
| | | | | 6.11-6.00 (m, 2H), 5.12 (d, J = 3.6 Hz, 1H), 5.05 (d, J = 8.4 |
| | | | | Hz, 1H), 4.46-4.26 (m, 4H), 4.23-4.13 (m, 2H), 3.80-3.72 |
| | | | | (m, 1H), 3.61-3.53 (m, 1H), 3.50-3.38 (m, 2H), 2.97-2.85 |
| | | | | (m, 6H), 2.67-2.62 (m, 1H), 2.57 (s, 1H), 2.45 (s, 3H), 2.43 |
| | | | | (s, 1H), 2.37-2.29 (m, 2H), 2.26-2.11 (m, 4H), 2.07 (s, 1H), |
| | | | | 2.05-1.99 (m, 1H), 1.99-1.86 (m, 3H), 1.69 (d, J = 6.4 Hz, |
| | | | | 3H), 1.63-1.56 (m, 2H), 1.53 (d, J = 6.4 Hz, 3H), 1.45-1.39 |
| | | | | (m, 1H), 0.98-0.92 (m, 3H), 0.85-0.77 (m, 3H) |
| I-37 | B | AM | 1060.9 | 8.96-9.00 (m, 1 H), 8.72 (s, 1 H), 8.31-8.44 (m, 3 H), 8.20- |
| | | | | 8.24 (m, 1 H), 7.23-7.37 (m, 2 H), 6.91-7.09 (m, 4 H), |
| | | | | 6.13-6.19 (m, 1H), 6.05 (q, J = 6.40 Hz, 1 H), 5.01-5.10 (m, |
| | | | | 1 H), 4.88 (d, J = 2.40 Hz, 1 H), 4.80-4.85 (m, 1 H), 4.69- |
| | | | | 4.76 (m, 1 H), 4.21-4.49 (m, 4 H), 3.57 (s, 5 H), 2.84-3.05 |
| | | | | (m, 5 H), 2.41-2.49 (m, 5 H), 2.20-2.36 (m, 4 H), 1.93 (s, |
| | | | | 4 H), 1.50-1.80 (m, 14 H), 0.91-1.00 (m, 3 H), 0.76-0.86 |
| | | | | (m, 3 H) |
| I-38 | B | AM | 1060.9 | 8.96-9.02 (m, 1 H), 8.75 (s, 1 H), 8.27-8.39 (m, 3 H), 8.22 |
| | | | | (dd, J = 8.40, 1.20 Hz, 1 H), 7.23-7.37 (m, 2 H), 7.04-7.12 |
| | | | | (m, 1 H), 6.92-7.00 (m, 3 H), 6.12-6.21 (m, 1 H), 6.00- |
| | | | | 6.09 (m, 1H), 5.03-5.12 (m, 1H), 4.87-4.92 (m, 1H), 4.81- |
| | | | | 4.86 (m, 1 H), 4.12-4.48 (m, 5 H), 3.57 (s, 4 H), 2.88-3.01 |
| | | | | (m, 4 H), 2.45 (d, J = 2.40 Hz, 5 H), 2.23-2.30 (m, 3 H), 1.88- |
| | | | | 2.14 (m, 5 H), 1.71-1.86 (m, 4 H), 1.44-1.65 (m, 9 H), |
| | | | | 1.19-1.33 (m, 3 H), 0.91-1.02 (m, 3 H), 0.79-0.86 (m, 3 H). |
| I-39 | O | AM | 1060.3 | 12.46 (s, 1H), 8.98 (d, J = 1.60 Hz, 1 H), 8.67-8.74 (m, 1 H), |
| | | | | 8.30-8.44 (m, 3 H), 8.18-8.22 (m, 1 H), 7.27-7.39 (m, 2 |
| | | | | H), 6.94-7.06 (m, 4 H), 6.10-6.20 (m, 1 H), 5.99-6.09 (m, |
| | | | | 1 H), 5.00-5.19 (m, 2 H), 4.85-4.90 (m, 1 H), 4.76-4.81 |
| | | | | (m, 1 H), 4.26-4.50 (m, 4 H), 3.52-3.83 (m, 4 H), 3.39- |
| | | | | 3.49 (m, 4 H), 2.87-2.97 (m, 3 H), 2.41-2.47 (m, 4H), 1.51- |
| | | | | 2.25 (m, 22 H), 0.95 (dd, J = 8.40, 6.80 Hz, 3 H), 0.74-0.86 |
| | | | | (m, 3 H) |
| I-40 | O | AM | 1060.3 | 12.46 (s, 1 H), 8.93-9.12 (m, 1 H), 8.71 (s, 1 H), 8.29-8.41 |
| | | | | (m, 3 H), 8.19-8.24 (m, 1 H), 7.24-7.38 (m, 2 H), 7.06- |
| | | | | 7.16 (m, 1 H), 6.92-7.02 (m, 3 H), 6.12-6.20 (m, 1 H), 6.00- |
| | | | | 6.10 (m, 1 H), 5.01-5.19 (m, 2 H), 4.80-4.93 (m, 2 H), |
| | | | | 4.33-4.52 (m, 3 H), 4.15-4.32 (m, 2 H), 3.54-3.86 (m, 3 |
| | | | | H), 3.47 (d, J = 12.0 Hz, 5 H), 2.86-3.04 (m, 3 H), 2.43-2.48 |
| | | | | (m, 4 H), 2.15-2.34 (m, 4 H), 2.00-2.12 (m, 3 H), 1.83- |
| | | | | 1.96 (m, 5 H), 1.40-1.72 (m, 9 H), 0.97 (dd, J = 11.2, 6.63 |
| | | | | Hz, 3 H), 0.75-0.87 (m, 3 H) |
| I-41 | AQ | AL | 1035.4 | 12.45 (s, 1H), 8.99 (s, 1H), 8.72 (s, 1H), 8.55-8.44 (m, 1H), |
| | | | | 8.34-8.33 (m, 2H), 8.25 (s, 1H), 8.21 (dd, J = 1.2, 8.4 Hz, |
| | | | | 1H), 7.46-7.42 (m, 2H), 7.41-7.36 (m, 2H), 7.36-7.27 (m, |
| | | | | 2H), 6.98-6.94 (m, 2H), 6.12-6.06 (m, 1H), 6.06-6.01 (m, |
| | | | | 1H), 5.08-5.03 (m, 1H), 4.37-4.29 (m, 3H), 4.24-4.21 (m, |
| | | | | 1H), 4.17 (t, J = 5.6 Hz, 1H), 3.79-3.75 (m, 1H), 3.67 (d, J = |
| | | | | 9.6 Hz, 1H), 2.97-2.91 (m, 6H), 2.46-2.45 (m, 3H), 2.44 |
| | | | | (d, J = 1.2 Hz, 1H), 2.28-2.15 (m, 6H), 2.06-1.90 (m, 5H), |
| | | | | 1.74-1.67 (m, 4H), 1.66-1.57 (m, 3H), 1.54 (d, J = 6.4 Hz, |
| | | | | 3H), 1.50-1.35 (m, 3H), 0.98-0.94 (m, 3H), 0.85-0.79 (m, 3H) |
| I-42^{d} | EH | EG | 986.5 | 13.92 (s, 1H) 11.54 (s, 1H) 9.18 (d, J = 2.4 Hz, 1H) 8.76 (s, |
| | | | | 1H) 8.73 (s, 1H) 8.69 (s, 1H) 8.65-8.52 (m, 1H) 7.64 (d, J = |
| | | | | 6.8 Hz, 1H) 7.51-7.37 (m, 2H) 7.31-7.19 (m, 2H) 7.08- |
| | | | | 7.04 (m, 2H) 7.02-6.91 (m, 1H) 6.19 (d, J = 6 Hz, 1H) 5.29 |
| | | | | (d, J = 14 Hz, 2H) 4.94 (s, 1H) 4.63-4.49 (m, 2H) 4.37-4.32 |
| | | | | (m, 2H) 4.26 (d, J = 4.4 Hz, 2H) 4.21-4.12 (m, 1H) 3.70- |
| | | | | 3.58 (m, 2H) 3.51-3.40 (m, 2H) 3.34-2.98 (m, 6H) 2.45 (d, |
| | | | | J = 3.2 Hz, 3H) 2.36-2.20 (m, 2H) 2.10 (d, J = 10 Hz, 3H) |
| | | | | 1.96-1.88 (m, 1H) 1.57 (d, J = 6.4 Hz, 3H) 1.43-1.27 (m, |
| | | | | 2H) 1.26-1.16 (m, 2H) 0.95 (s, 9H) |
| I-43^{d} | EH | EG | 986.4 | 13.93 (s, 1H) 11.54 (s, 1H) 9.18 (d, J = 2.4 Hz, 1H) 8.76 (s, |
| | | | | 1H) 8.73 (s, 1H) 8.69 (s, 1H) 8.64-8.52 (m, 1H) 7.65 (d, J = |
| | | | | 7.8 Hz, 1H) 7.51-7.41 (m, 2H) 7.31-7.19 (m, 2H) 7.14- |
| | | | | 7.04 (m, 2H) 7.02-6.95 (m, 1H) 6.19 (d, J = 6.6 Hz, 1H) |
| | | | | 5.16 (d, J = 11.8 Hz, 2H) 4.94 (s, 1H) 4.62-4.57 (m, 2H) 4.57- |
| | | | | 48 (m, 2H) 4.26 (d, J = 4.4 Hz, 2H) 4.21-4.12 (m, 1H) 3.70- |
| | | | | 3.58 (m, 2H) 3.51 (m, 2H) 3.34-2.98 (m, 6H) 2.45 (d, J = |
| | | | | 3.2 Hz, 3H) 2.36-2.20 (m, 2H) 2.10 (d, J = 10 Hz, 3H) 1.96- |
| | | | | 1.88 (m, 1H) 1.57 (d, J = 6.4 Hz, 3H) 1.43-1.27 (m, 2H) |
| | | | | 1.26-1.16 (m, 2H) 0.95 (s, 9H) |
| I-44^{c, e} | EJ | EI | 1055.6 | 14.19-14.51 (m, 1H), 12.31-12.68 (m, 1H), 8.99 (s, 1H), |
| | | | | 8.71-8.71 (m, 1H), 8.68-8.76 (m, 1H), 8.47-8.56 (m, 1H), |
| | | | | 8.36-8.46 (m, 1H), 8.34 (s, 2H), 8.17-8.26 (m, 1H), 7.42 |
| | | | | (d, J = 40 Hz, 1H), 7.24-7.35 (m, 2H), 6.89-7.01 (m, 3H), |
| | | | | 6.76 (s, 1H), 5.99-6.10 (m, 1H), 5.01-5.11 (m, 1H), 4.85- |
| | | | | 4.96 (m, 1H), 4.56-4.64 (m, 1H), 4.48-4.55 (m, 1H), 4.34- |
| | | | | 4.39 (m, 1H), 4.18-4.32 (m, 2H), 3.74-3.85 (m, 2H), 3.56- |
| | | | | 3.69 (m, 3H), 2.86-3.13 (m, 7H), 2.55-2.64 (m, 2H), 2.45- |
| | | | | 2.47 (m, 3H), 2.27-2.40 (m, 4H), 2.06-2.15 (m, 1H), 1.88- |
| | | | | 2.04 (m, 3H), 1.59-1.75 (m, 4H), 1.51-1.57 (m, 3H), 1.31- |
| | | | | 1.43 (m, 2H), 1.17-1.27 (m, 2H), 0.96 (s, 9H) |
| I-45^{c} | EA | AY | 1032.6 | 8.97 (s, 1H), 8.50 (t, J = 5.6 Hz, 1H), 8.24 (s, 2H), 7.95 (d, J = |
| | | | | 8.0 Hz, 1H), 7.44-7.39 (m, 2H), 7.34-7.27 (m, 2H), 7.25- |
| | | | | 7.19 (m, 1H), 7.03-6.99 (m, 1H), 6.95 (dd, J = 1.2, 7.8 Hz, |
| | | | | 1H), 6.90-6.83 (m, 2H), 5.18 (d, J = 3.2 Hz, 1H), 4.66-4.57 |
| | | | | (m, 3H), 4.52 (t, J = 8.4 Hz, 1H), 4.38-4.33 (m, 1H), 4.29 |
| | | | | (d, J = 5.6 Hz, 1H), 4.26-4.17 (m, 2H), 4.10 (br t, J = 6.0 |
| | | | | Hz, 2H), 3.70-3.57 (m, 3H), 3.27-3.23 (m, 1H), 3.11-2.92 |
| | | | | (m, 6H), 2.71-2.65 (m, 1H), 2.55-2.52 (m, 4H), 2.47-2.45 |
| | | | | (m, 4H), 2.40 (t, J = 6.8 Hz, 2H), 2.09 (dd, J = 8.4, 11.6 Hz, |
| | | | | 1H), 1.99-1.88 (m, 1H), 1.86-1.75 (m, 2H), 1.72-1.60 (m, |
| | | | | 2H), 1.42-1.37 (m, 1H), 1.37-1.33 (m, 1H), 1.22 (dd, J = |
| | | | | 3.2, 8.3 Hz, 2H), 0.96 (s, 9H) |
| I-46^{c} | AV | AY | 516.5^{b} | 8.98 (s, 1H), 8.50 (t, J = 5.8 Hz, 1H), 8.31 (d, J = 5.2 Hz, |
| | | | | 1H), 7.94 (d, J = 7.2 Hz, 1H), 7.47-7.39 (m, 2H), 7.33-7.27 |
| | | | | (m, 2H), 7.24-7.19 (m, 1H), 7.03-7.00 (m, 1H), 6.98-6.94 |
| | | | | (m, 1H), 6.90-6.83 (m, 2H), 6.61 (d, J = 5.2 Hz, 1H), 4.77 |
| | | | | (d, J = 13.2 Hz, 2H), 4.60 (d, J = 9.2 Hz, 1H), 4.53 (t, J = 8.0 |
| | | | | Hz, 1H), 4.43-4.31 (m, 2H), 4.30-4.22 (m, 2H), 4.18 (d, J = |
| | | | | 13.6 Hz, lH), 4.10 (t, J = 5.6 Hz, 2H), 3.66-3.60 (m, 3H), |
| | | | | 3.19-3.13 (m, 2H), 2.98 (d, J = 11.6 Hz, 2H), 2.96-2.96 (m, |
| | | | | 1H), 2.69-2.66 (m, 1H), 2.39 (t, J = 6.8 Hz, 3H), 2.17-2.04 |
| | | | | (m, 2H), 2.02-1.93 (m, 3H), 1.81 (d, J = 9.2 Hz, 4H), 1.74- |
| | | | | 1.59 (m, 5H), 1.44-1.27 (m, 3H), 1.25-1.19 (m, 2H), 0.95 |
| | | | | (s, 9H) |
| I-47 | AQ | N | 1138.0 | 14.33 (s, 1H), 12.44 (s, 1H), 8.97 (s, 1H), 8.71 (s, 1H), 8.50 |
| | | | | (t, J = 6.0 Hz, 1H), 8.33 (s, 2H), 8.21 (dd, J = 1.2, 8.4 Hz, |
| | | | | 1H), 7.41 (d, J = 7.6 Hz, 1H), 7.32-7.26 (m, 2H), 7.01 (s, |
| | | | | 1H), 6.98-6.90 (m, 3H), 6.07-6.01 (m, 1H), 5.18 (d, J = 3.6 |
| | | | | Hz, 1H), 5.08-5.01 (m, 1H), 4.71 (s, 1H), 4.60 (d, J = 9.6 |
| | | | | Hz, 1H), 4.52 (t, J = 8.0 Hz, 1H), 4.35 (d, J = 1.0 Hz, 1H), |
| | | | | 4.31-4.27 (m, 1H), 3.68-3.58 (m, 2H), 3.41 (s, 1H), 3.00- |
| | | | | 2.93 (m, 4H), 2.92 (s, 1H), 2.44 (s, 3H), 2.41-2.35 (m, 2H), |
| | | | | 2.26-2.17 (m, 4H), 2.10 (d, J = 5.6 Hz, 1H), 2.03-1.93 (m, |
| | | | | 3H), 1.73 (d, J = 8.4 Hz, 4H), 1.59 (s, 7H), 1.54 (d, J = 6.4 |
| | | | | Hz, 4H), 1.45 (d, J = 2.0 Hz, 1H), 1.42-1.33 (m, 3H), 1.23 |
| | | | | (s, 4H), 1.21 (s, 1H), 0.97 (s, 9H) |
| I-48 | AQ | N | 1138.0 | 14.37 (s, 1H), 12.45 (s, 1H), 8.98 (s, 1H), 8.71 (s, 1H), 8.45 |
| | | | | (t, J = 5.6 Hz, 1H), 8.33-8.32 (m, 2H), 8.23-8.18 (m, 1H), |
| | | | | 7.38 (d, J = 7.6 Hz, 1H), 7.32-7.25 (m, 2H), 7.05 (s, 1H), |
| | | | | 6.98-6.91 (m, 3H), 6.04 (q, J = 6.4 Hz, 1H), 5.05 (dd, J = |
| | | | | 4.0, 13.6 Hz, 1H), 4.59 (d, J = 9.2 Hz, 1H), 4.50 (t, J = 8.4 |
| | | | | Hz, 1H), 4.35 (s, 2H), 4.27-4.15 (m, 2H), 3.67-3.57 (m, |
| | | | | 3H), 2.98-2.84 (m, 7H), 2.45 (s, 3H), 2.35 (s, 1H), 2.23- |
| | | | | 2.14 (m, 4H), 2.14-2.06 (m, 5H), 1.94-1.88 (m, 1H), 1.77 |
| | | | | (s, 2H), 1.71 (d, J = 10.4 Hz, 4H), 1.64-1.57 (m, 2H), 1.54 |
| | | | | (d, J = 6.4 Hz, 3H), 1.44-1.37 (m, 6H), 1.37-1.31 (m, 2H), |
| | | | | 1.22 (dd, J = 2.7, 8.3 Hz, 2H), 0.98-0.94 (m, 9H) |
| I-49^{c} | M | AY | 1032.0 | 8.98 (s, 1H), 8.50 (t, J = 6.0 Hz, 1H), 8.33 (s, 2H), 7.95 (d, J = |
| | | | | 8.0 Hz, 1H), 7.43-7.38 (m, 2H), 7.33-7.26 (m, 2H), 7.24- |
| | | | | 7.19 (m, 1H), 7.03-6.99 (m, 1H), 6.98-6.94 (m, 1H), 6.90- |
| | | | | 6.84 (m, 2H), 4.72 (d, J = 13.2 Hz, 2H), 4.60 (d, J = 9.2 Hz, |
| | | | | 1H), 4.52 (t, J = 8.4 Hz, 1H), 4.38-4.33 (m, 1H), 4.29 (d, J = |
| | | | | 6.0 Hz, 1H), 4.26-4.17 (m, 2H), 4.09 (t, J = 6.0 Hz, 2H), |
| | | | | 3.69-3.58 (m, 4H), 3.27-3.23 (m, 2H), 3.18-3.09 (m, 2H), |
| | | | | 3.04-2.94 (m, 3H), 2.76 (dd, J = 10.8, 12.8 Hz, 1H), 2.47 (s, |
| | | | | 3H), 2.40 (t, J = 6.8 Hz, 3H), 2.13-2.05 (m, 1H), 2.02-1.89 |
| | | | | (m, 3H), 1.84-1.75 (m, 2H), 1.74-1.59 (m, 6H), 1.42-1.37 |
| | | | | (m, 1H), 1.37-1.32 (m, 1H), 1.22 (dd, J = 3.2, 8.3 Hz, 2H), |
| | | | | 0.96 (s, 9H) |

^{a}Step 1 was run anywhere starting from 0° C. to raising the temp to 25-50° C. The Step 2 SFC separation was performed with a variety of solvent conditions to separate the chiral compounds. Absolute stereochemistry of the compounds was assigned arbitrarily.
^{b}LCMS data reported as (M/2 + H)⁺.
^{c}No Step 2 SFC separation of the coupled product.
^{d}After SFC separation the isomer was deprotected with HCl/dioxane in DCM at 25° C. for 1 hour. Then the final compound was purified via reverse phase HPLC (HCl condition: column: Phenomenex Luna C18 150* 25 mm* 10 um; mobile phase: [water(0.05% HCl)-ACN]; B %: 17%-47%, 10 min).
^{e}NaBH(OAc)₃ in THF at 40° C. for 12 hrs was used for the reductive amination in Step 1.

TABLE 4A
Compounds synthesized via Method 2 using the corresponding amines
and aldehydes or ketones for the reductive amination.

| | | Aldehyde/ | LCMS (ESI+) | |
|---|---|---|---|---|
| I-#^{a} | Amine | Ketone | m/z (M + H)⁺ | ¹H NMR (400 MHz, DMSO-d6) |
| I-89^{b} | KG | JF | 983.4 | 14.33 (s, 1 H), 12.37-12.54 (m, 1 H), 8.71 (s, 1 H), 8.59 (t, |
| | | | | J = 6.0 Hz, 1 H), 8.33 (s, 2 H), 8.21 (dd, J = 8.4, 1.20 Hz, 1 H), |
| | | | | 7.36-7.47 (m, 2 H), 7.25-7.35 (m, 3 H), 6.91-7.02 (m, 2 |
| | | | | H), 6.04 (q, J = 6.0 Hz, 1 H), 4.99-5.16 (m, 2 H), 4.54 (t, J = 8.4 |
| | | | | Hz, 1 H), 4.35-4.43 (m, 1 H), 4.19-4.27 (m, 1 H), 4.11- |
| | | | | 4.17 (m, 1 H), 3.87-3.95 (m, 2 H), 3.70-3.81 (m, 2 H), 3.63 |
| | | | | dd, J = 11.6, 2.8 Hz, 1 H), 2.88-3.05 (m, 3 H), 2.83 (d, J = 9.6 |
| | | | | Hz, 2 H), 2.65-2.76 (m, 1 H), 2.31-2.43 (m, 2 H), 2.23 (dd, |
| | | | | J = 12.8, 7.2 Hz, 3 H), 2.16 (s, 3 H), 2.05 (ddd, J = 13.6, 9.2, 4.4 |
| | | | | Hz, 1 H), 1.87-1.95 (m, 2 H), 1.66-1.80 (m, 5 H), 1.47- |
| | | | | 1.63 (m, 6H), 0.91 (s, 9 H) |
| I-90^{b} | JX | JF | 983.6 | 8.53 (t, J = 5.6 Hz, 1H), 8.21 (s, 2H), 7.93 (d, J = 7.6 Hz, 1H), |
| | | | | 7.68 (d, J = 8.4 Hz, 1H), 7.38-7.27 (m, 6H), 7.21 (t, J = 7.6 |
| | | | | Hz, 1H), 6.89-6.83 (m, 2H), 4.48 (d, J = 8.8 Hz, 2H), 4.42- |
| | | | | 4.37 (m, 2H), 4.33 (s, 1H), 4.19 (d, J = 4.8 Hz, 1H), 4.16- |
| | | | | 4.11 (m, 3H), 3.64 (d, J = 6.8 Hz, 3H), 3.59 (d, J = 10.4 Hz, |
| | | | | 2H), 3.22-3.15 (m, 2H), 2.98 (d, J = 12.0 Hz, 1H), 2.89-2.79 |
| | | | | (m, 6H), 2.54 (s, 2H), 2.36-2.28 (m, 2H), 2.11 (d, J = 3.2 |
| | | | | Hz, 1H), 2.03-1.94 (m, 2H), 1.88 (dd, J = 4.4, 8.7 Hz, 1H), |
| | | | | 1.80-1.68 (m, 7H), 1.55-1.34 (m, 10H), 1.27 (d, J = 2.8 Hz, |
| | | | | 3H), 0.92 (s, 9H) |
| I-91^{b} | JR | JF | 983.7 | 8.52 (t, J = 6.0 Hz, 1H), 8.21 (s, 2H), 7.92 (d, J = 7.6 Hz, 1H), |
| | | | | 7.67 (d, J = 9.2 Hz, 1H), 7.39-7.26 (m, 6H), 7.24-7.18 (m, |
| | | | | 1H), 6.90-6.82 (m, 2H), 4.48 (d, J = 8.8 Hz, 2H), 4.42-4.36 |
| | | | | (m, 2H), 4.33 (s, 1H), 4.19 (d, J = 5.6 Hz, 1H), 4.16-4.11 (m, |
| | | | | 3H), 3.65 (dd, J = 3.6, 10.4 Hz, 3H), 3.59 (d, J = 10.0 Hz, 2H), |
| | | | | 3.34-3.31 (m, 1H), 3.22-3.15 (m, 2H), 2.90-2.80 (m, 6H), |
| | | | | 2.54-2.52 (m, 2H), 2.29 (dd, J = 2.8, 7.2 Hz, 2H), 2.11 (t, J = |
| | | | | 9.2 Hz, 1H), 2.04-1.92 (m, 2H), 1.87 (ddd, J = 4.8, 8.4, |
| | | | | 12.8 Hz, 1H), 1.78-1.64 (m, 7H), 1.58-1.22 (m, 13H), 0.92 |
| | | | | (s, 9H) |
| I-92^{b} | KI | JF | 955.6 | 0.92 (s, 9 H) 1.03-1.18 (m, 3 H) 1.20-1.33 (m, 4 H) 1.40 (d, |
| | | | | J = 7, 2 Hz, 2 H) 1.45-1.53 (m, 3 H) 1.60 (d, J = 10.0 Hz, 3 H) |
| | | | | 1.67 (s, 1 H) 1.76 (s, 3 H) 1.86-1.97 (m, 3 H) 1.98-2.03 (m, |
| | | | | 1 H) 2.24-2.37 (m, 2 H) 2.68 (dd, J = 3.60, 1.63 Hz, 1 H) 2.79- |
| | | | | 2.94 (m, 3 H) 3.02-3.07 (m, 1 H) 3.09-3.16 (m, 3 H) 3.19 |
| | | | | (d, J = 7.2 Hz, 2 H) 3.21-3.21 (m, 1 H) 3.23 (s, 1 H) 3.64 (d, |
| | | | | J = 4.00 Hz, 2 H) 3.73-3.83 (m, 2 H) 4.11-4.17 (m, 2 H) 4.19 |
| | | | | (d, J = 5.6 Hz, 1 H) 4.34 (s, 1 H) 4.37-4.40 (m, 1 H) 4.43 (s, 1 |
| | | | | H) 4.49 (d, J = 9.6 Hz, 1 H) 6.83-6.90 (m, 2 H) 7.19-7.25 (m, |
| | | | | 1 H) 7.29 (d, J = 4.8 Hz, 1 H) 7.31-7.34 (m, 2 H) 7.35-7.41 |
| | | | | (m, 3 H) 7.69 (d, J = 9.2 Hz, 1 H) 7.93 (d, J = 8.00 Hz, 1 H) 8.26 |
| | | | | (s, 1 H) 8.55 (t, J = 6.00 Hz, 1 H) |
| I-93 | HC | HD | 1045.2 | 8.99 (s, 1H) 8.56 (t, J = 8.0 Hz, 1H) 7.91 (dd, J = 8.0, 4.0 Hz, |
| | | | | 1H) 7.74 (br d, J = 8.0 Hz, 1H) 7.43-7.38 (m, 5H) 7.27 (s, |
| | | | | 1H) 7.24-7.20 (m, 1H) 6.89-6.85 (m, 2H) 5.14 (d, J = 4.0 |
| | | | | Hz, 1H) 4.51 (d, J = 12.0 Hz, 1H) 4.47-4.40 (m, 2H) 4.36 (br |
| | | | | s, 1H) 4.25-4.19 (m, 1H) 4.09 (br d, J = 12.0 Hz, 1H) 3.71- |
| | | | | 3.65 (m, 5H) 3.62-3.77 (m, 2H) 3.24-3.16 (m, 3H) 3.12 (br |
| | | | | s, 2H) 3.02-2.95 (m, 2H) 2.70-2.64 (m, 1H) 2.56-2.50 (m, |
| | | | | 1H) 2.45 (s, 3H) 2.35-2.30 (m, 1H) 2.10-2.00 (m, 3H) 1.92- |
| | | | | 1.87(m, 2H) 1.69 (br d, J = 12.0 Hz, 2H) 1.59-1.50 (m, 2H) |
| | | | | 1.42-1.39 (m, 1H) 1.35-1.29 (m, 4H) 1.24-1.16 (m, 1H) |
| | | | | 0.93 (s, 9H) |
| I-94^{c, d} | HX | HD | 511.9 | 8.99 (s, 1H) 8.56 (t, J = 4.0 Hz, 1H) 8.05 (d, J = 2.0 Hz, 1H) |
| | | | | 7.95 (d, J = 7.6 Hz, 1H) 7.71 (br d, J = 9.2 Hz, 1H) 7.52 (dd, |
| | | | | J = 8.8, 2.0 Hz, 1H) 7.45-7.36 (m, 6H) 7.32 (s, 1H) 7.24- |
| | | | | 7.20 (m, 1H) 6.93 (d, J = 8.8 Hz, 1H) 6.89-6.85 (m, 2H) 5.14 |
| | | | | (d, J = 3.6 Hz, 1H) 4.51 (d, J = 9.2 Hz, 1H) 4.47-4.45 (m, |
| | | | | 1H) 4.41-4.36 (m, 2H) 4.37 (br s, 2H) 4.25-4.20 (m, 1H) |
| | | | | 4.19-4.17 (m, 1H) 3.68-3.59 (m, 3H) 3.27-3.23 (m, 2H) |
| | | | | 2.99 (d, J = 8.0 Hz, 2H) 2.93-2.86 (m, 2H) 2.65-2.58 (m, |
| | | | | 2H) 2.53 (br d, J = 1.6 Hz, 2H) 2.45 (s, 3H) 2.35-2.32 (m, |
| | | | | 1H) 2.05-1.97 (m, 2H) 1.90 (br s, 1H) 1.80 (br s, 1H) 1.75- |
| | | | | 1.66 (m, 5H) 1.61-1.49 (m, 5H) 1.45-1.40 (m, 2H) 1.32- |
| | | | | 1.24 (m, 3H) 0.93 (s, 9H) |
| I-95^{b} | KK | JF | 948.9 | 8.55 (t, J = 5.60 Hz, 1 H), 8.32 (s, 1 H), 7.98 (d, J = 8.00 Hz, 1 |
| | | | | H), 7.70 (d, J = 9.20 Hz, 1 H), 7.15-7.45 (m, 10 H), 6.76-6.94 |
| | | | | (m, 2 H), 4.47-4.55 (m, 1 H), 4.30-4.45 (m, 3 H), 4.11-4.23 |
| | | | | (m, 2 H), 4.06 (d, J = 12.80 Hz, 1 H), 3.73-3.88 (m, 2 H), 3.62- |
| | | | | 3.68 (m, 2 H), 3.02-3.12 (m, 2 H), 2.85-2.97 (m, 3 H), 2.60- |
| | | | | 2.79 (m, 2H), 2.21-2.42 (m, 4H), 1.93-2.10 (m, 3 H), 1.88 |
| | | | | (ddd, J = 12.80, 8.80, 4.40 Hz, 2 H), 1.63-1.81 (m, 7 H), 1.39- |
| | | | | 1.59 (m, 6 H), 1.20-1.37 (m, 3 H), 0.86-1.01 (m, 9 H) |
| I-102^{c} | HH | ID | 968.6 | 8.49-8.64 (m, 1 H), 8.35 (s, 2 H), 7.80 (d, J = 8.20 Hz, 1 H), |
| | | | | 7.70 (d, J = 9.20 Hz, 1 H), 7.52 (d, J = 3.20 Hz, 1 H), 7.35-7.35 |
| | | | | (m, 1 H), 7.26-7.44 (m, 4 H), 7.17 (dd, J = 10.40, 8.40 Hz, 1 |
| | | | | H), 6.82 (td, J = 8.00, 5.20 Hz, 1 H), 5.13 (d, J = 2.80 Hz, 1 H), |
| | | | | 4.72 (t, J = 10.40 Hz, 2 H), 4.50 (d, J = 9.20 Hz, 1 H), 4.30-4.46 |
| | | | | (m, 3 H), 4.10-4.25 (m, 3 H), 3.57-3.70 (m, 3 H), 3.29 (s, 2 |
| | | | | H), 3.09-3.20 (m, 1 H), 2.83-3.04 (m, 3 H), 2.72-2.81 (m, |
| | | | | 1 H), 2.61 (t, J = 7, 60 Hz. 1 H), 2.25-2.45 (m, 3 H), 1.94-2.08 |
| | | | | (m, 2 H), 1.78-1.92 (m, 2 H), 1.61-1.75 (m, 6 H), 1.38-1.75 |
| | | | | (m, 9 H), 1.21-1.36 (m, 3 H), 0.93 (s, 8 H) |
| I-122^{c} | EA | JF | 951.6 | 14.84-14.60 (m, 1H), 8.54 (t, J = 6.0 Hz, 1H), 8.24 (s, 2H), |
| | | | | 7.94 (d, J = 8.0 Hz, 1H), 7.69 (d, J = 9.6 Hz, 1H), 7.44-7.28 |
| | | | | (m, 7H), 7.21 (t, J = 7.6 Hz, 1H), 6.90-6.83 (m, 2H), 5.12 (d, |
| | | | | J = 3.1 Hz, 1H), 4.62 (d, J = 12.4 Hz, 2H), 4.50 (d, J = 9.2 Hz, |
| | | | | 1H), 4.43-4.33 (m, 3H), 4.21-4.11 (m, 3H), 3.67-3.59 (m, |
| | | | | 3H), 3.29-3.22 (m, 2H), 3.09-2.92 (m, 7H), 2.69-2.65 (m, |
| | | | | 2H), 2.37 (s, 2H), 2.05-1.97 (m, 2H), 1.90-1.78 (m, 2H), |
| | | | | 1.72 (d, J = 12.4 Hz, 1H), 1.57-1.39 (m, 7H), 1.34-1.21 (m, |
| | | | | 4H), 0.92 (s, 9H) |
| I-133 | HC | HD | 1044.7 | 8.99 (s, 1H) 8.56 (t, J = 8.0 Hz, 1H) 7.92 (dd, J = 8.0, 4.0 Hz, |
| | | | | 1H) 7.72 (br d, J = 8.0 Hz, 1H) 7.43-7.38 (m, 5H) 7.27 (s, |
| | | | | 1H) 7.24-7.20 (m, 1H) 6.89-6.85 (m, 2H) 5.14 (d, J = 4.0 |
| | | | | Hz, 1H) 4.51 (d, J = 8.0 Hz, 1H) 4.47-4.40 (m, 2H) 4.36 (br |
| | | | | s, 1H) 4.25-4.19 (m, 1H) 4.09 (br d, J = 8.0 Hz, 1H) 3.71- |
| | | | | 3.65 (m, 5H) 3.62-3.77 (m, 2H) 3.24-3.16 (m, 3H) 3.12 (br |
| | | | | s, 2H) 3.02-2.95 (m, 2H) 2.70-2.64 (m, 1H) 2.56-2.50 (m, |
| | | | | 1H) 2.45 (s, 3H) 2.35-2.30 (m, 1H) 2.10-2.00 (m, 3H) 1.92- |
| | | | | 1.87(m, 2H) 1.69 (br d, J = 12.0 Hz, 2H) 1.59-1.50 (m, 2H) |
| | | | | 1.42-1.39 (m, 1H) 1.31-1.21 (m, 4H) 1.06-1.04 (m, 1H) |
| | | | | 0.93 (s, 9H) |
| I-148^{c, d, e} | HX | HD | 511.9 | 8.99 (s, 1H) 8.56 (t, J = 4.0 Hz, 1H) 8.05 (d, J = 2.0 Hz, 1H) |
| | | | | 7.95 (d, J = 7.6 Hz, 1H) 7.71 (br d, J = 9.2 Hz, 1H) 7.51 (dd, |
| | | | | J = 8.8, 2.0 Hz, 1H) 7.46-7.37 (m, 6H) 7.32 (s, 1H) 7.25- |
| | | | | 7.20 (m, 1H) 6.93 (d, J = 8.8 Hz, 1H) 6.88-6.85 (m, 2H) 5.14 |
| | | | | (d, J = 3.6 Hz, 1H) 4.51 (d, J = 9.2 Hz, 1H) 4.48-4.45 (m, |
| | | | | 1H) 4.41-4.35 (m, 2H) 4.37 (br s, 2H) 4.25-4.20 (m, 1H) |
| | | | | 4.19-4.17 (m, 1H) 3.68-3.59 (m, 3H) 3.28-3.23 (m, 2H) |
| | | | | 2.99 (d, J = 8.0 Hz, 2H) 2.93-2.86 (m, 2H) 2.65-2.58 (m, |
| | | | | 2H) 2.53 (br d, J = 1.6 Hz, 2H) 2.45 (s, 3H) 2.35-2.32 (m, |
| | | | | 1H) 2.05-1.97 (m, 2H) 1.90 (br s, 1H) 1.80 (br s, 1H) 1.75- |
| | | | | 1.66 (m, 5H) 1.61-1.49 (m, 5H) 1.45-1.41 (m, 2H) 1.32- |
| | | | | 1.24 (m, 3H) 0.93 (s, 9H) |
| I-155^{c} | HX | JF | 949.6 | 8.57-8.52 (m, 1H) 8.05 (d, J = 1.6 Hz, 1H) 7.95 (br d, J = 7.6 |
| | | | | Hz, 1H) 7.70 (br d, J = 9.2 Hz, 1 H) 7.52 (br dd, J = 8.8, 1.6 |
| | | | | Hz, 1H) 7.40-7.37 (m, 2H) 7.33 (br d, J = 7.6 Hz, 4H) 7.24- |
| | | | | 7.19 (m, 1H) 6.93 (br d, J = 8.0 Hz, 1H) 6.89-6.84 (m, 2H) |
| | | | | 5.12 (d, J = 3.2 Hz, 1H) 4.50 (br d, J = 9.6 Hz, 1H) 4.42-4.37 |
| | | | | (m, 4H) 4.34 (br d, J = 1.2 Hz, 1H) 4.21-4.15 (m, 2H) 4.14- |
| | | | | 4.12 (m, 1H) 3.68-3.57 (m, 4H) 3.25 (br d, J = 1.6 Hz, 1H) |
| | | | | 2.99 (br d, J = 9.2 Hz, 2H) 2.94-2.84 (m, 3H) 2.68 (br s, 2H) |
| | | | | 2.61 (br s, 2H) 2.39-2.30 (m, 3H) 2.04-1.96 (m, 2H) 1.73- |
| | | | | 1.69 (m, 5H) 1.60 (br t, J = 12.0 Hz, 3H) 1.54-1.49 (m, 2H) |
| | | | | 1.41 (br d, J = 11.6 Hz, 3H) 1.33-1.24 (m, 3H) 0.93 (s, 9H) |
| I-156^{c} | HX | JF | 949.6 | 8.55 (br t, J = 5.6 Hz, 1H) 8.05 (d, J = 1.6 Hz, 1H) 7.96 (br d, |
| | | | | J = 7.6 Hz, 1H) 7.70 (br d, J = 9.2 Hz, 1H) 7.52 (dd, J = 8.8, |
| | | | | 1.6 Hz, 1H) 7.41-7.37 (m, 2H) 7.36-7.30 (m, 4H) 7.22 (t, J = |
| | | | | 7.6 Hz, 1H) 6.93 (br d, J = 8.8 Hz, 1H) 6.90-6.82 (m, 2H) |
| | | | | 5.13 (d, J = 3.6 Hz, 1H) 4.50 (d, J = 9.6 Hz, 1H) 4.44-4.33 |
| | | | | (m, 5H) 4.22-4.15 (m, 2H) 4.14 (s, 1H) 3.69-3.58 (m, 3H) |
| | | | | 3.31-3.22 (m, 2H) 3.03-2.96 (m, 2H) 2.89 (br t, J = 10.0 |
| | | | | Hz, 2H) 2.65-2.57 (m, 3H) 2.39 (br t, J = 11.2 Hz, 1H) 2.30 |
| | | | | (br d, J = 2.4 Hz, 1H) 2.05-1.95 (m, 2H) 1.88-1.84 (m, 1H) |
| | | | | 1.79 (br d, J = 5.6 Hz, 1H) 1.69 (br d, J = 12.0 Hz, 5H) 1.64- |
| | | | | 1.55 (m, 3H) 1.51 (br dd, J = 10.0, 8.0 Hz, 3H) 1.46-1.39 (m, |
| | | | | 2H) 1.33-1.23 (m, 3H) 0.93 (s, 9H). |
| I-170^{c} | EA | JF | 951.6 | 14.72 (s, 1H), 8.54 (t, J = 6.0 Hz, 1H), 8.24 (s, 2H), 7.94 (d, J = |
| | | | | 7.6 Hz, 1H), 7.69 (d, J = 9.2 Hz, 1H), 7.48-7.27 (m, 7H), |
| | | | | 7.25-7.18 (m, 1H), 6.90-6.81 (m, 2H), 5.11 (d, J = 3.1 Hz, |
| | | | | 1H), 4.62 (d, J = 12.0 Hz, 2H), 4.49 (d, J = 9.2 Hz, 1H), 4.44- |
| | | | | 4.33 (m, 3H), 4.23-4.12 (m, 3H), 3.67-3.57 (m, 3H), 3.30- |
| | | | | 3.22 (m, 2H), 3.11-2.91 (m, 7H), 2.69-2.65 (m, 2H), 2.36 |
| | | | | (s, 2H), 1.99 (d, J = 8.0 Hz, 2H), 1.90-1.78 (m, 2H), 1.72 (d, |
| | | | | J = 12.0 Hz, 1H), 1.64-1.25 (m, 11H), 0.93 (s, 9H) |
| I-195^{c} | EA | LX | 981.6 | 14.86-14.59 (m, 1H), 8.40 (t, J = 5.6 Hz, 1H), 8.24 (s, 2H), |
| | | | | 7.99-7.89 (m, 1H), 7.74-7.64 (m, 1H), 7.42-7.37 (m, 1H), |
| | | | | 7.35 (d, J = 7.6 Hz, 1H), 7.31 (s, 1H), 7.25-7.17 (m, 1H), |
| | | | | 7.02 (d, J = 1.2 Hz, 1H), 6.96-6.90 (m, 1H), 6.90-6.82 (m, |
| | | | | 2H), 5.16-5.05 (m, 1H), 4.67-4.57 (m, 2H), 4.53-4.46 (m, |
| | | | | 1H), 4.43 (t, J = 8.0 Hz, 1H), 4.37-4.30 (m, 1H), 4.29-4.15 |
| | | | | (m, 2H), 4.15-4.06 (m, 2H), 3.81 (s, 3H), 3.70-3.56 (m, 3H), |
| | | | | 3.29 (s, 1H), 3.12-2.89 (m, 7H), 2.37 (s, 4H), 2.09-1.93 (m, |
| | | | | 3H), 1.92-1.85 (m, 1H), 1.83-1.78 (m, 1H), 1.76-1.69 (m, |
| | | | | 1H), 1.60-1.38 (m, 7H), 1.34-1.21 (m, 4H), 0.91 (s, 9H) |
| I-196^{c} | EA | LX | 981.6 | 14.89-14.57 (m, 1H), 8.45-8.35 (m, 1H), 8.24 (s, 2H), 7.97- |
| | | | | 7.91 (m, 1H), 7.63 (s, 1H), 7.39 (d, J = 3.2 Hz, 1H), 7.35 (d, |
| | | | | J = 7.6 Hz, 1H), 7.31 (s, 1H), 7.24-7.17 (m, 1H), 7.02 (s, 1H), |
| | | | | 6.96-6.90 (m, 1H), 6.89-6.81 (m, 2H), 5.14-5.07 (m, 1H), |
| | | | | 4.62 (d, J = 11.6 Hz, 2H), 4.49 (d, J = 9.6 Hz, 1H), 4.42 (t, J = |
| | | | | 8.0 Hz, 1H), 4.36-4.30 (m, 1H), 4.29-4.16 (m, 2H), 4.15- |
| | | | | 4.07 (m, 2H), 3.81 (s, 3H), 3.68-3.56 (m, 3H), 3.29 (s, 1H), |
| | | | | 3.12-2.90 (m, 7H), 2.37 (s, 4H), 2.06-1.93 (m, 3H), 1.92- |
| | | | | 1.85 (m, 1H), 1.83-1.77 (m, 1H), 1.75-1.69 (m, 1H), 1.65- |
| | | | | 1.58 (m, 1H), 1.56-1.49 (m, 2H), 1.49-1.40 (m, 3H), 1.39- |
| | | | | 1.34 (m, 1H), 1.33-1.21 (m, 4H), 0.91 (s, 9H) |
| I-197^{b} | HH | LY | 926.4 | 14.36 (s, 1 H), 12.25 (s, 1 H), 8.41-8.65 (m, 2 H), 8.11 (d, |
| | | | | J = 7.60 Hz, 1 H), 7.73 (d, J = 9.20 Hz, 1 H), 7.21-7.43 (m, 5 |
| | | | | H), 6.87-7.04 (m, 2 H), 5.12 (d, J = 3.20 Hz, 1 H), 4.51 (d, |
| | | | | J = 9.60 Hz, 1 H), 4.36-4.43 (m, 2 H), 4.28-4.35 (m, 2 H), |
| | | | | 4.09-4.21 (m, 2 H), 3.57-3.68 (m, 2 H), 3.02-3.12 (m, 2 |
| | | | | H), 2.72-2.92 (m, 5 H), 2.62-2.72 (m, 1 H), 2.52-2.58 (m, |
| | | | | 3 H), 2.30-2.38 (m, 1 H), 2.09-2.23 (m, 3 H), 1.96-2.06 |
| | | | | (m, 3 H), 1.87 (ddd, J = 12.80, 8.40, 4.40 Hz, 2 H), 1.69-1.82 |
| | | | | (m, 6H), 1.65 (d, J = 13.20 Hz, 1 H), 1.47-1.59 (m, 4 H), 1.41- |
| | | | | 1.46 (m, 2H), 1.37 (d, J = 6.40 Hz, 4 H), 1.09-1.26 (m, 2 H), |
| | | | | 0.92 (s, 8 H) |
| I-201^{f} | MA | MB | 843.8 | 14.36 (s, 1H), 12.27 (s, 1H), 8.57-8.51 (m, 2H), 8.16-8.09 |
| | | | | (m, 1H), 7.75 (d, J = 9.2 Hz, 1H), 7.39-7.35 (m, 2H), 7.34- |
| | | | | 7.30 (m, 2H), 7.30-7.26 (m, 1H), 6.99-6.92 (m, 2H), 5.12 |
| | | | | (d, J = 3.2 Hz, 1H), 4.52 (d, J = 9.2 Hz, 1H), 4.44-4.37 (m, |
| | | | | 2H), 4.36-4.32 (m, 1H), 4.30 (d, J = 6.0 Hz, 1H), 4.21-4.15 |
| | | | | (m, 1H), 4.13 (s, 1H), 3.70-3.57 (m, 2H), 3.31-3.28 (m, 2H), |
| | | | | 3.16-3.07 (m, 1H), 3.04-2.97 (m, 1H), 2.86-2.77 (m, 3H), |
| | | | | 2.73-2.67 (m, 1H), 2.66-2.57 (m, 1H), 2.53 (d, J = 2.0 Hz, |
| | | | | 1H), 2.38-2.30 (m, 1H), 2.09-2.00 (m, 3H), 1.98-1.92 (m, |
| | | | | 1H), 1.89-1.76 (m, 6H), 1.66 (d, J = 10.4 Hz, 1H), 1.59-1.50 |
| | | | | (m, 3H), 1.40 (d, J = 6.4 Hz, 3H), 1.33 (d, J = 12.4 Hz, 1H), |
| | | | | 1.28-1.20 (m, 1H), 0.93 (s, 9H), 0.91-0.85 (m, 2H) |
| I-202^{f} | MA | MB | 843.8 | 14.45 . 14.32 (m, 1H), 12.32-12.22 (m, 1H), 8.55 (s, 2H), |
| | | | | 8.12 (d, J = 6.4 Hz, 1H), 7.75 (d, J = 9.2 Hz, 1H), 7.40-7.25 |
| | | | | (m, 5H), 6.99-6.91 (m, 2H), 5.12 (d, J = 2.4 Hz, 1H), 4.55- |
| | | | | 4.48 (m, 1H), 4.44-4.32 (m, 4H), 4.23-4.10 (m, 2H), 3.68- |
| | | | | 3.58 (m, 2H), 3.17-3.09 (m, 1H), 3.05-2.97 (m, 1H), 2.83 |
| | | | | (d, J = 4.4 Hz, 3H), 2.70-2.62 (m, 3H), 2.33 (s, 2H), 2.13 (d, |
| | | | | J = 1.2 Hz, 2H), 2.07-1.96 (m, 2H), 1.88-1.74 (m, 5H), 1.71- |
| | | | | 1.63 (m, 2H), 1.56-1.45 (m, 6H), 1.36 (d, J = 5.2 Hz, 4H), |
| | | | | 1.24 (d, J = 1.2 Hz, 1H), 0.93 (s, 9H) |
| I-206 | MD | MF | 925.6 | 12.32 (s, 1H), 8.54-8.50 (m, 2H), 8.14 (s, 1H), 8.10 (d, J = |
| | | | | 7.2 Hz, 1H), 7.38-7.36 (m, 2H), 7.32 (s, 2H), 7.29-7.26 (m, |
| | | | | 1H), 6.96 (d, J = 8.0 Hz, 2H), 5.99 (br d, J = 8.8 Hz, 1H), 4.42- |
| | | | | 4.38 (m, 2H), 4.36 (br d, J = 7.2 Hz, 3H), 4.18 (br dd, J = |
| | | | | 5.6, 15.6 Hz, 1H), 4.12 (s, 1H), 4.00-3.94 (m, 2H), 3.83 (br |
| | | | | s, 1H), 3.73 (br s, 2H), 3.67 (br s, 2H), 3.10 (br dd, J = 4.0, 4.0 |
| | | | | Hz, 2H), 2.98-2.85 (m, 6H), 2.74-2.66 (m, 4H), 2.54 (s, 1H), |
| | | | | 2.37-2.32 (m, 2H), 2.17-2.08 (m, 4H), 2.04-1.95 (m, 2H), |
| | | | | 1.90-1.85 (m, 2H), 1.74 (br s, 2H), 1.60-1.52 (m, 2H), 1.40 |
| | | | | (br d, J = 6.4 Hz, 3H), 1.12 (br s, 1H), 1.08 (br d, J = 5.6 Hz, |
| | | | | 1H), 0.93 (s, 9H) |
| I-209 | MG | MF | 937.4 | 8.54 (t, J = 6.0 Hz, 1H), 8.48 (s, 1H), 8.13-8.06 (m, 1H), 7.39- |
| | | | | 7.36 (m, 2H), 7.33-7.30 (m, 2H), 7.26 (t, J = 7.6 Hz, 1H), |
| | | | | 6.95-6.90 (m, 2H), 5.78 (d, J = 9.2 Hz, 1H), 4.45-4.30 (m, |
| | | | | 6H), 4.22-4.16 (m, 1H), 4.14-4.08 (m, 2H), 3.79-3.61 (m, |
| | | | | 6H), 3.14-3.08 (m, 2H), 3.06-2.99 (m, 5H), 2.95 (d, J = 13.2 |
| | | | | Hz, 4H), 2.89-2.83 (m, 4H), 2.28-2.17 (m, 3H), 2.03-1.94 |
| | | | | (m, 5H), 1.80-1.70 (m, 3H), 1.52 (d, J = 4.0 Hz, 3H), 1.33- |
| | | | | 1.24 (m, 4H), 0.93 (s, 9H) |
| I-213^{c} | MX | HL | 941.5 | 8.35 (d, J = 7.6 Hz, 1H), 7.92 (d, J = 1.2 Hz, 1H), 7.57 (d, J = |
| | | | | 9.2 Hz, 1H), 7.42 (d, J = 8 Hz, 2H), 7.33 (d, J = 3.2 Hz, 1H), |
| | | | | 7.29 (d, J = 8.4 Hz, 2H), 7.23-7.19 (m, 2H), 6.87-6.83 (m, |
| | | | | 2H), 5.09 (d, J = 3.6 Hz, 1H), 4.87 (t, J = 7.2 Hz, 1H), 4.48 (d, |
| | | | | J = 9.2 Hz, 1H), 4.39 (t, J = 8.0 Hz, 1H), 4.27 (d, J = 2.4 Hz, |
| | | | | 1H), 4.12 (s, 1H), 4.04 (d, J = 12.4 Hz, 1H), 3.62-3.56 (m, |
| | | | | 2H), 3.49-3.44 (m, 1H), 3.23-3.13 (m, 3H), 3.11 (d, J = 8.4 |
| | | | | Hz, 1H), 3.05 (d, J = 8.4 Hz, 2H), 2.89 (d, J = 7.2 Hz, 2H), |
| | | | | 2.82 (d, J = 12.2 Hz, 1H), 2.79-2.72 (m, 2H), 2.32-2.26 (m, |
| | | | | 1H), 2.19-2.07 (m, 6H), 2.05-1.85 (m, 8H), 1.79-1.72 (m, |
| | | | | 4H), 1.68-1.51 (m, 6H), 1.38 (s, 2H), 1.33 (d, J = 7.2 Hz, |
| | | | | 3H), 1.24 (s, 1H), 0.91 (s, 9H) |
| I-226^{c} | MX | HL | 941.6 | 8.36 (d, J = 8 Hz, 1H), 7.92 (d, J = 7.1 Hz, 1H), 7.62-7.54 |
| | | | | (m, 1H), 7.41 (d, J = 8.2 Hz, 2H), 7.35 (s, 1H), 7.29 (d, J = 8.0 |
| | | | | Hz, 2H), 7.23-7.18 (m, 2H), 6.89-6.82 (m, 2H), 5.14-5.06 |
| | | | | (m, 1H), 4.89-4.82 (m, 1H), 4.51-4.44 (m, 1H), 4.39 (t, J = |
| | | | | 8.0 Hz, 1H), 4.29-4.24 (m, 1H), 4.13 (s, 1H), 4.04 (d, J = |
| | | | | 12.0 Hz, 1H), 3.58 (s, 2H), 3.49-3.42 (m, 2H), 3.18-3.14 |
| | | | | (m, 2H), 3.06-3.01 (m, 2H), 2.89 (d, J = 8.1 Hz, 2H), 2.81- |
| | | | | 2.72 (m, 4H), 2.32-2.23 (m, 2H), 2.14 (d, J = 3.8 Hz, 4H), |
| | | | | 2.12-2.03 (m, 6H), 2.01-1.80 (m, 6H), 1.78-1.71 (m, 4H), |
| | | | | 1.70-1.55 (m, 6H), 1.33 (d, J = 6.9 Hz, 3H), 0.91 (s, 9H) |
| I-243 | PR | PG | 943.7 | 8.56 (t, J = 6.0 Hz, 1H), 8.18 (d, J = 3.2 Hz, 1H), 7.91 (d, J = |
| | | | | 8.4 Hz, 1H), 7.85 (d, J = 9.6 Hz, 1H), 7.38-7.31 (m, 5H), |
| | | | | 7.24-7.18 (m, 2H), 6.89-6.84 (m, 2H), 4.54 (d, J = 9.6 Hz, |
| | | | | 1H), 4.43 (d, J = 6.4 Hz, 1H), 4.42-4.37 (m, 2H), 4.34 (s, |
| | | | | 1H), 4.19 (d, J = 5.6 Hz, 1H), 4.16-4.14 (m, 1H), 4.11-4.01 |
| | | | | (m, 2H), 3.69-3.63 (m, 3H), 3.18-3.14 (m, 3H), 2.98-2.85 |
| | | | | (m, 6H), 2.82 (d, J = 11.2 Hz, 1H), 2.65-2.60 (m, 2H), 2.30- |
| | | | | 2.22 (m, 4H), 2.14-2.08 (m, 2H), 2.04-1.93 (m, 4H), 1.92- |
| | | | | 1.84 (m, 2H), 1.74-1.66 (m, 2H), 1.60-1.56 (m, 1H), 1.40- |
| | | | | 1.40 (m, 1H), 1.55-1.39 (m, 5H), 1.25 (s, 8H), 0.94 (s, 9H) |
| I-246^{c} | PC | JF | 929.7 | 8.53 (t, J = 6.0 Hz, 1H), 7.92 (d, J = 7.2 Hz, 1H), 7.67 (d, J = |
| | | | | 9.2 Hz, 1H), 7.38-7.29 (m, 5H), 7.24-7.17 (m, 2H), 6.88- |
| | | | | 6.80 (m, 2H), 5.11 (d, J = 3.6 Hz, 1H), 4.48 (d, J = 9.2 Hz, |
| | | | | 1H), 4.43-4.36 (m, 2H), 4.33 (s, 1H), 4.21-4.10 (m, 2H), |
| | | | | 4.02 (d, J = 11.6 Hz, 1H), 3.68-3.57 (m, 2H), 3.50-3.39 (m, |
| | | | | 2H), 3.16 (d, J = 10.8 Hz, 2H), 3.04 (d, J = 10.8 Hz, 2H), 2.82 |
| | | | | (d, J = 11.2 Hz, 3H), 2.59-2.55 (m, 2H), 2.43 (d, J = 9.2 Hz, |
| | | | | 1H), 2.41-2.36 (m, 1H), 2.31-2.24 (m, 2H), 2.19 (s, 3H), |
| | | | | 2.16-2.10 (m, 1H), 2.04-1.98 (m, 1H), 1.95-1.85 (m, 2H), |
| | | | | 1.82-1.74 (m, 2H), 1.68-1.55 (m, 6H), 1.51-1.43 (m, 3H), |
| | | | | 1.38 (d, J = 11.2 Hz, 5H), 1.30-1.19 (m, 3H), 0.92 (s, 9H) |
| I-247^{c} | PC | JF | 929.6 | 8.53 (t, J = 6.0 Hz, 1H), 7.92 (d, J = 7.2 Hz, 1H), 7.68 (d, J = |
| | | | | 8.8 Hz, 1H), 7.38-7.29 (m, 5H), 7.24-7.17 (m, 2H), 6.89- |
| | | | | 6.81 (m, 2H), 5.11 (d, J = 3.2 Hz, 1H), 4.49 (d, J = 9.6 Hz, |
| | | | | 1H), 4.39 (t, J = 8.4 Hz, 2H), 4.33 (s, 1H), 4.17 (dd, J = 5.6, |
| | | | | 16.4 Hz, 1H), 4.12 (s, 1H), 4.03 (d, J = 11.6 Hz, 1H), 3.67- |
| | | | | 3.57 (m, 2H), 3.50-3.41 (m, 2H), 3.17 (d, J = 10.4 Hz, 2H), |
| | | | | 3.05 (d, J = 10.0 Hz, 2H), 2.92-2.81 (m, 3H), 2.64-2.57 (m, |
| | | | | 2H), 2.26 (d, J = 1.2 Hz, 4H), 2.16 (t, J = 10.4 Hz, 2H), 2.08- |
| | | | | 1.92 (m, 3H), 1.91-1.82 (m, 2H), 1.78 (d, J = 10.4 Hz, 2H), |
| | | | | 1.73-1.64 (m, 4H), 1.58-1.36 (m, 9H), 1.34-1.18 (m, 4H), |
| | | | | 1.16-1.11 (m, 1H), 0.92 (s, 9H) |
| I-251 | PR | OQ | 915.5 | 8.55 (t, J = 6.0 Hz, 1H), 8.22 (s, 2H), 7.91 (d, J = 13.6 Hz, |
| | | | | 1H), 7.84 (d, J = 9.4 Hz, 1H), 7.40-7.32 (m, 5H), 7.24-7.18 |
| | | | | (m, 2H), 6.90-6.82 (m, 2H), 4.53 (d, J = 12 Hz, 2H), 4.44- |
| | | | | 4.32 (m, 4H), 4.20 (d, J = 4.8 Hz, 1H), 4.17-4.12 (m, 2H), |
| | | | | 4.05 (d, J = 3.2 Hz, 2H), 3.68-3.65 (m, 3H), 3.15-3.11 (m, |
| | | | | 3H), 2.91 (d, J = 8.4 Hz, 3H), 2.84-2.79 (m, 3H), 2.29-2.19 |
| | | | | (m, 5H), 2.15-2.05 (m, 3H), 2.02-1.87 (m, 8H), 1.63-1.55 |
| | | | | (m, 3H), 1.51-1.35 (m, 5H), 1.25-1.15 (m, 4H), 0.94 (s, 9H) |

^{a}The reductive coupling was run under standard conditions from 0° C.-25° C. over 10 min-4 hrs. SFC or HPLC separation was performed with a variety of solvent conditions to separate the chiral compounds. Absolute stereochemistry of the compounds was assigned arbitrarily.
^{b}Cis-trans mixture at spirocycle.
^{c}Chiral center on spirocycle arbitrarily assigned.
^{d}LC-MS (ESI⁺) m/z = (M/2 + H)⁺.
^{e}The product of the coupling was further separated by prep-HPLC (neutral condition; column: Waters xbridge 150* 25 mm 10 um; mobile phase: [water (NH4HCO3)-ACN]; B %: 33%-63%, 11 min) then further purified by prep-HPLC (neutral condition; column: Waters xbr idge 150* 25 mm 10 um; mobile phase: [water (NH₄HCO₃)-ACN]; B %: 30%-60%, 11 min).
^{f}The product of the coupling was then deprotected with TsOH in H₂O and MeOH at 50° C. for 3 hrs. The final product was then separated by prep-HPLC. Absolute stereochemistry of the diastereomers was arbitrarily assigned.

### Example 3 (Method 3): Synthesis of (2S,4R)-4-hydroxy-1-(2-(3-(3-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoyl)-N-(4-(4-methylthiazol-5-yl)benzyl)pyrrolidine-2-carboxamide (I-52)

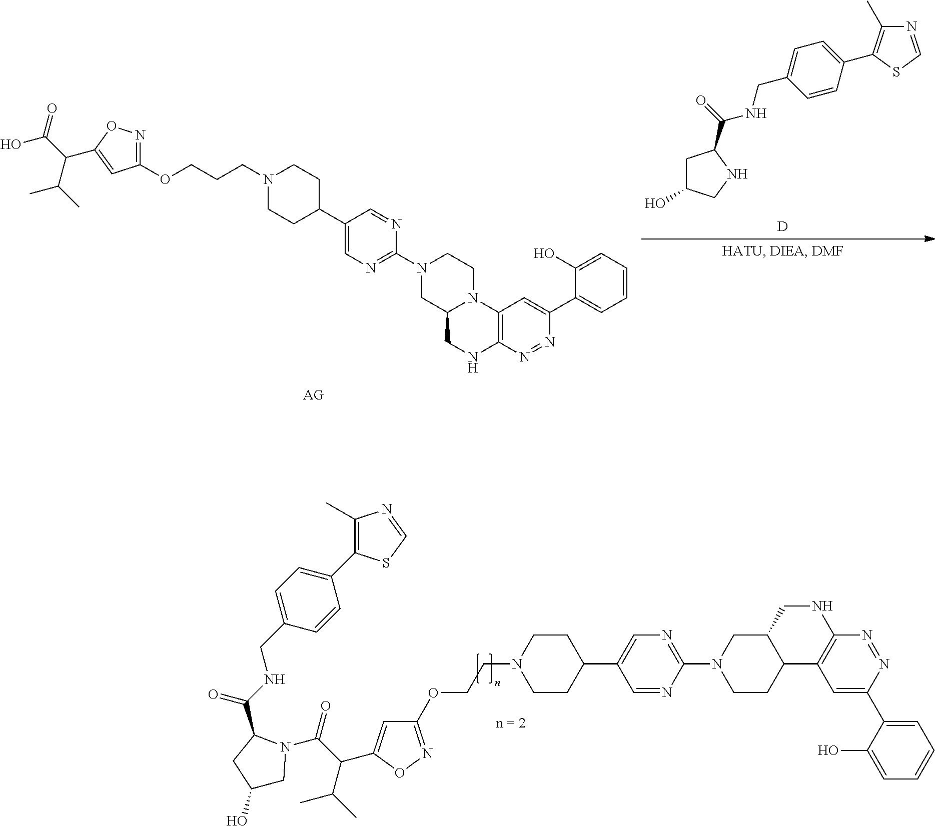

To a solution of 2-(3-(3-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)propoxy)isoxazol-5-yl)-3-methylbutanoic acid (60.0 mg, 89.6 umol, Intermediate AG) in DMF (1 mL) was added HATU (37.5 mg, 98.5 umol), (2S,4R)-4-hydroxy-N-[[4-(4-methylthiazol-5-yl)phenyl]methyl]pyrrolidine-2-carboxamide (31.7 mg, 89.6 umol, Intermediate D) and DIEA (57.9 mg, 448 umol). The mixture was stirred at 25° C. for 5 minutes. On completion, the reaction mixture was filtered and concentrated in vacuo to get the crude residue. The crude product was purified by reversed-phase HPLC (column: Welch Xtimate C18 150×25 mm×5 um; mobile phase: [water(0.05% HCl)-ACN]; B %: 12%-42%, 10 min). The residue was then further purified by reversed-phase HPLC (column: 3 Phenomenex Luna C18 75×30 mm×3 um; mobile phase: [water (10 mM NH₄HCO₃)-ACN]; B %: 28%-58%, 10 min) to give the title compound (15.7 mg, 18% yield) as yellow solid. ¹H NMR (400 MHz, DMSO-d6) δ=8.99 (d, J=3.6 Hz, 1H), 8.54 (q, J=5.6 Hz, 1H), 8.34 (br d, J=3.2 Hz, 2H), 7.89 (br d, J=8.0 Hz, 1H), 7.49 (br s, 1H), 7.44-7.41 (m, 2H), 7.39-7.33 (m, 2H), 7.30 (s, 1H), 7.23 (br t, J=7.6 Hz, 1H), 6.90-6.86 (m, 2H), 6.11 (d, J=10.4 Hz, 1H), 5.17 (br s, 1H), 4.71 (br t, J=9.6 Hz, 3H), 4.51-4.41 (m, 1H), 4.35-4.19 (m, 8H), 3.81-3.75 (m, 1H), 3.65-3.58 (m, 2H), 3.53-3.47 (m, 2H), 3.15 (br d, J=11.6 Hz, 3H), 3.00 (br d, J=12.0 Hz, 2H), 2.83-2.75 (m, 2H), 2.45 (s, 3H), 2.29-2.12 (m, 5H), 1.96 (br s, 5H), 1.23 (br s, 1H), 0.96 (br t, J=7.2 Hz, 3H), 0.82 (br dd, J=6.8, 13.2 Hz, 3H). LC-MS (ESI⁺) m/z 969.3 (M+H)⁺.

TABLE 5
Compounds synthesized via Method 3 using the corresponding amines and acids for the coupling

| | | | LCMS (ESI+) | |
|---|---|---|---|---|
| I-#^{a} | Amine | Acid | m/z (M + H)⁺ | ¹H NMR (400 MHz, DMSO-d6) |
| I-53 | D | AI | 969.4 | 14.77-14.67 (m, 1H), 8.98 (s, 1H), 8.35-8.33 (m, 2H), 7.94 (d, J = |
| | | | | 8.0 Hz, 1H), 7.43-7.36 (m, 5H), 7.32 (d, J = 10.0 Hz, 2H), 7.21 |
| | | | | (t, J = 7.2 Hz, 1H), 6.88-6.84 (m, 2H), 6.07-6.05 (m, 1H), 5.14- |
| | | | | 5.10 (m, 1H), 4.71 (br dd, J = 4.8, 8.0 Hz, 2H), 4.43 (t, J = 8.0 |
| | | | | Hz, 1H), 4.36-4.31 (m, 2H), 4.30-4.27 (m, 1H), 4.21-4.17 (m, |
| | | | | 2H), 4.13 (br t, J = 6.4 Hz, 2H), 3.64-3.55 (m, 3H), 3.16-3.08 |
| | | | | (m, 2H), 2.94 (dt, J = 8.8, 12.0 Hz, 4H), 2.78-2.72 (m, 1H), 2.68- |
| | | | | 2.66 (m, 1H), 2.45 (s, 3H), 2.36 (br d, J = 7.6 Hz, 2H), 2.33-2.32 |
| | | | | (m, 1H), 2.30-2.21 (m, 2H), 2.07-2.01 (m, 1H), 1.93-1.90 (m, |
| | | | | 2H), 1.89-1.87 (m, 1H), 1.84-1.81 (m, 1H), 1.70-1.65 (m, 3H), |
| | | | | 1.62-1.58 (m, 1H), 0.98-0.94 (m, 3H), 0.84-0.79 (m, 3H) |
| I-54^{b} | D | EN | 1006.5 | 9.02-8.99 (m, 1H), 8.44 (t, J = 6.0 Hz, 1H), 8.35 (s, 2H), 7.95 (d, |
| | | | | J = 7.6 Hz, 1H), 7.48-7.39 (m, 4H), 7.36-7.30 (m, 3H), 7.25- |
| | | | | 7.19 (m, 1H), 6.91-6.83 (m, 2H), 5.84 (s, 1H), 5.80 (s, 1H), 4.76- |
| | | | | 4.68 (m, 2H), 4.44 (t, J = 7.6 Hz, 1H), 4.38-4.26 (m, 3H), 4.25- |
| | | | | 4.18 (m, 1H), 3.89 (s, 1H), 3.84-3.75 (m, 2H), 3.73-3.68 (m, |
| | | | | 2H), 3.65-3.60 (m, 1H), 3.57 (d, J = 2. Hz, 1H), 3.46 (s, 1H), |
| | | | | 3.27-3.21 (m, 2H), 3.20-3.10 (m, 2H), 3.01-2.92 (m, 2H), 2.77 |
| | | | | (t, J = 11.2 Hz, 1H), 2.47 (s, 4H), 2.45 (s, 2H), 2.10-1.98 (m, 4H), |
| | | | | 1.95-1.87 (m, 2H), 1.83-1.75 (m, 2H), 1.71-1.56 (m, 3H), 0.96 |
| | | | | (d, J = 6.4 Hz, 3H), 0.82 (d, J = 6.8 Hz, 3H), 0.67 (d, J = 7.2 Hz, |
| | | | | 1H), 0.59 (d, J = 6.4 Hz, 1H) |
| I-55^{b} | D | EN | 1006.5 | 9.01-8.98 (m, 1H), 8.51 (t, J = 5.6 Hz, 1H), 8.35 (s, 2H), 8.16 (s, |
| | | | | 1H), 7.95 (d, J = 8.0 Hz, 1H), 7.47-7.34 (m, 5H), 7.32 (s, 1H), |
| | | | | 7.25-7.19 (m, 1H), 6.90-6.84 (m, 2H), 5.83 (s, 1H), 4.76-4.68 |
| | | | | (m, 2H), 4.37-4.31 (m, 3H), 4.21 (d, J = 12.8 Hz, 1H), 3.88 (s, |
| | | | | 1H), 3.82-3.77 (m, 1H), 3.76 (s, 2H), 3.64-3.58 (m, 2H), 3.43- |
| | | | | 3.40 (m, 2H), 3.27-3.24 (m, 2H), 3.18-3.10 (m, 2H), 2.99-2.94 |
| | | | | (m, 1H), 2.88 (d, J = 9.6 Hz, 2H), 2.78 (d, J = 12.8 Hz, 1H), 2.60 |
| | | | | (d, J = 7.6 Hz, 1H), 2.46 (s, 3H), 2.45 (s, 1H), 2.40 (s, 1H), 2.34- |
| | | | | 2.30 (m, 2H), 2.25-2.21 (m, 1H), 2.05-1.89 (m, 4H), 1.82-1.69 |
| | | | | (m, 4H), 1.65-1.55 (m, 2H), 0.94 (d, J = 6.4 Hz, 3H), 0.79 (d, J = |
| | | | | 6.4 Hz, 3H) |
| I-56^{c} | EZ | EM | 964.6 | 8.34 (d, J = 2.8 Hz, 3H), 7.94 (d, J = 6.9 Hz, 1H), 7.63 (d, J = 7.6 |
| | | | | Hz, 1H), 7.45-7.36 (m, 3H), 7.29 (dd, J = 4.6, 7.3 Hz, 3H), 7.21 |
| | | | | (t, J = 8.1 Hz, 1H), 6.91-6.81 (m, 2H), 5.09 (s, 1H), 4.87 (dt, J = |
| | | | | 2.3, 6.9 Hz, 1H), 4.76-4.67 (m, 2H), 4.50-4.44 (m, 1H), 4.39 (dt, |
| | | | | J = 2.6, 7.4 Hz, 1H), 4.26 (d, J = 1.1 Hz, 1H), 4.20 (d, J = 11.0 Hz, |
| | | | | 1H), 4.12 (d, J = 3.8 Hz, 1H), 3.61-3.56 (m, 2H), 3.25 (s, 1H), |
| | | | | 3.17-3.08 (m, 2H), 3.00-2.92 (m, 2H), 2.92-2.83 (m, 3H), 2.74 |
| | | | | (d, J = 10.9 Hz, 2H), 2.67 (d, J = 1.5 Hz, 1H), 2.40-2.37 (m, 1H), |
| | | | | 2.33 (d, J = 2.1 Hz, 1H), 2.27 (dd, J = 2.0, 4.6 Hz, 1H), 2.00-1.94 |
| | | | | (m, 2H), 1.77 (dd, J = 1.3, 3.2 Hz, 1H), 1.74-1.64 (m, 6H), 1.60 |
| | | | | (d, J = 11.0 Hz, 1H), 1.52-1.46 (m, 3H), 1.54 (s, 1H), 1.42 (d, J = |
| | | | | 9.4 Hz, 2H), 1.35-1.31 (m, 3H), 1.29 (s, 2H), 0.92 (s, 9H) |
| I-57^{c} | EZ | EM | 964.6 | 8.38-8.33 (m, 3H), 7.94 (d, J = 7.9 Hz, 1H), 7.63 (d, J = 9.0 Hz, |
| | | | | 1H), 7.43-7.38 (m, 3H), 7.32-7.26 (m, 3H), 7.21 (t, J = 7.7 Hz, |
| | | | | 1H), 6.91-6.83 (m, 2H), 5.09 (d, J = 3.4 Hz, 1H), 4.87 (t, J = 7.3 |
| | | | | Hz, 1H), 4.74-4.68 (m, 2H), 4.46 (d, J = 9.3 Hz, 1H), 4.39 (t, J = |
| | | | | 8.1 Hz, 1H), 4.26 (s, 1H), 4.20 (d, J = 11.9 Hz, 1H), 4.12 (s, 1H), |
| | | | | 3.58 (d, J = 7.0 Hz, 2H), 3.30 (s, 2H), 3.25 (s, 1H), 3.13 (d, J = 1.6 |
| | | | | Hz, 1H), 2.95 (dt, J = 2.9, 12.6 Hz, 2H), 2.90-2.84 (m, 2H), 2.79- |
| | | | | 2.72 (m, 2H), 2.38 (s, 1H), 2.30-2.24 (m, 1H), 2.07 (s, 1H), 2.00- |
| | | | | 1.94 (m, 2H), 1.78 (d, J = 10.1 Hz, 1H), 1.75-1.67 (m, 6H), 1.60 |
| | | | | (d, J = 11.3 Hz, 3H), 1.49 (d, J = 10.3 Hz, 2H), 1.44-1.37 (m, |
| | | | | 3H), 1.33 (d, J = 7.0 Hz, 3H), 1.27 (s, 2H), 0.92 (s, 9H) |

^{a}The reaction was run anywhere from 0-25 C. for 5 min to 16 hrs. Absolute stereochemistry of the separated diastereomers was assigned arbitrarily.
^{b}The product of the coupling was then separated and purified by SFC (column: DAICEL CHIRALPAK AD(250 mm × 30 mm 10 um); mobile phase: [0.1% NH₃H₂O IPA]; B %: 82%-82%, 12; 130 min). Then each isomer was further purified by prep-HPLC (column: Phenomenex luna C18 150 × 25 mm × 10 um; mobile phase: [water(0.225% FA)-ACN]; B %: 10%-40%, 11.5 min).
^{c}The coupled product was further separated by prep-HPLC(column: Phenomenex C18 150*25 mm*10 um; mobile phase: [water (NH₃H₂O)-ACN]; B %: 55%-75%, 10 min) to give the two diastereomers.

### Example 4 (Method 4): Synthesis of N—((S)-1-((2S,4R)-4-hydroxy-2-((4-(4-methylthiazol-5-yl)benzyl)carbamoyl)pyrrolidin-1-yl)-3,3-dimethyl-1l-oxobutan-2-yl)-6-(4-(2-((R)-3-(2-hydroxyphenyl)-5-methyl-7,8-dihydro-5H-pyrido[3′,4′: 4,5]pyrrolo[2,3-c]pyridazin-6(9H)-yl)pyrimidin-5-yl)piperidin-1-yl)-2-azaspiro[3.3]heptane-2-carboxamide (I-58)

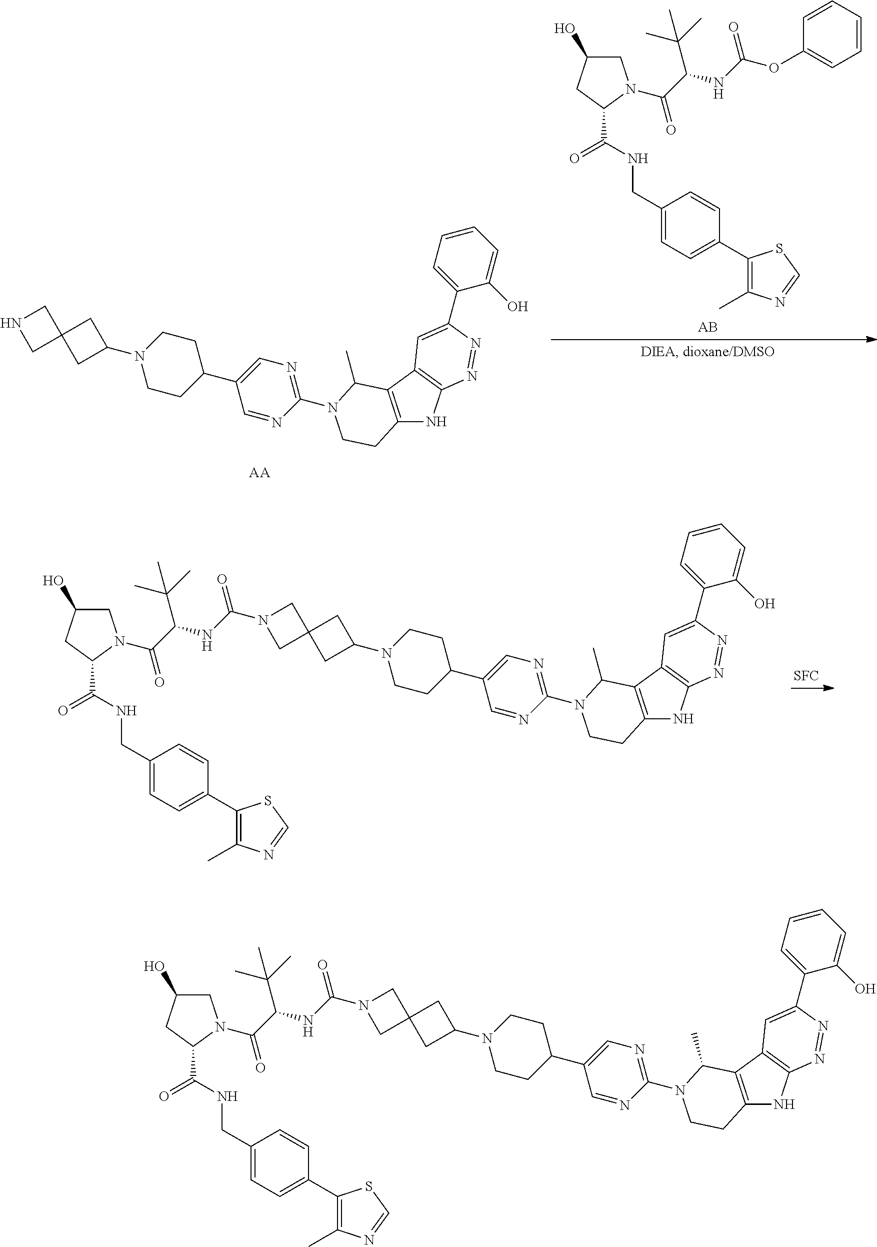 [SMILES: CC1c2c([nH]c3nnc(-c4ccccc4O)cc23)CCN1c1ncc(C2CCN(C3CC4(CNC4)C3)CC2)cn1 ; Cc1ncsc1-c1ccc(CNC(=O)[C@@H]2C[C@@H](O)CN2C(=O)[C@@H](NC(=O)Oc2ccccc2)C(C)(C)C)cc1 ; "AA" ; Cc1ncsc1-c1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7C6C)nc5)CC4)C3)C2)C(C)(C)C)cc1 ; "SFC" ; Cc1ncsc1-c1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)cc1]

Step 1-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-6-(4-(2-(3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxamide. To a solution of 2-[4-[5-[1-(2-azaspiro[3.3]heptan-6-yl)-4-piperidyl]pyrimidin-2-yl]-3-methyl-4,8,10,11-tetrazatricyclo[7.4.0.0^{2,7}]trideca-1(9),2(7),10,12-tetraen-12-yl]phenol (2.2 g, 4.10 mmol, Intermediate AA) and phenyl N-[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]carbamate (2.03 g, 3.69 mmol, Intermediate AB) in dioxane (20 mL) and DMSO (2 mL) was added DIEA (1.59 g, 12.3 mmol). The mixture was then stirred at 60° C. for 12 hours. On completion, the reaction mixture was filtered and concentrated under reduced pressure to give a residue. The residue was purified by prep-HPLC (column: Phenomenex luna C18 250×50 mm×10 um; mobile phase: [water (0.225% FA)-ACN]; B %: 25%-40%, 12 min) to give the title compound (2.1 g, 47% yield) as a yellow solid. LC-MS (ESI⁺) m/z 993.3 (M+H)⁺.

Step 2-N—((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-6-(4-(2-((R)-3-(2-hydroxyphenyl)-5-methyl-7,8-dihydro-5H-pyrido[3′,4′:4,5]pyrrolo[2,3-c]pyridazin-6(9H)-yl)pyrimidin-5-yl)piperidin-1-yl)-2-azaspiro[3.3]heptane-2-carboxamide. N-[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]-6-[4-[2-[12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-2-azaspiro[3.3]heptane-2-carboxamide (2.2 g, 2.22 mmol) was separated by SFC (column: DAICEL CHIRALPAK AS (250 mm×50 mm, 10 um); mobile phase: [0.1% NH₃H2O MEOH]; B %: 70%-70%, 12.2; 370 min). Then N-[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]-6-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-2-azaspiro[3.3]heptane-2-carboxamide (545 mg, 24% yield, FA) was further purified by prep-HPLC (column: Phenomenex Synergi Polar-RP 100×25 mm×4 um; mobile phase: [water (0.225% FA)-ACN]; B %: 18%-48%, 8 min) to give N-[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]-6-[4-[2-[(3R)-12-(2-hydroxyphenyl)-3-methyl-4,8,10,11-tetrazatricyclo[7.4.0.0^{2,7}]trideca-1(9),2(7),10,12-tetraen-4-yl]pyrimidin-5-yl]-1-piperidyl]-2-azaspiro[3.3]heptane-2-carboxamide (545 mg, 24% yield, FA) as a yellow solid. ¹H NMR (400 MHz, DMSO-d₆) δ=14.33 (s, 1H), 12.44 (s, 1H), 8.98 (s, 1H), 8.71 (s, 1H), 8.55 (t, J=6.0 Hz, 1H), 8.33 (s, 2H), 8.21 (d, J=8.0 Hz, 1H), 7.39 (s, 4H), 7.29 (t, J=7.6 Hz, 1H), 7.02-6.91 (m, 2H), 6.04 (q, J=6.4 Hz, 1H), 5.65 (d, J=9.2 Hz, 1H), 5.12 (d, J=3.2 Hz, 1H), 5.05 (dd, J=4.8, 12.8 Hz, 1H), 4.45-4.30 (m, 4H), 4.28-4.19 (m, 1H), 3.89-3.80 (m, 2H), 3.78-3.69 (m, 2H), 3.64 (s, 2H), 3.44-3.36 (m, 1H), 3.00-2.90 (m, 2H), 2.84 (d, J=9.6 Hz, 2H), 2.46-2.43 (m, 3H), 2.38-2.33 (m, 1H), 2.25 (d, J=8.8 Hz, 2H), 2.07-2.00 (m, 1H), 1.94-1.85 (m, 3H), 1.76-1.67 (m, 4H), 1.62-1.51 (m, 5H), 0.92 (s, 9H). LC-MS (ESI⁺) m/z 993.3 (M+H)⁺.

TABLE 6
Compounds synthesized via Method 4 using the corresponding
amines and carbamates for the coupling

| | | | LCMS (ESI+) | |
|---|---|---|---|---|
| I-#^{a} | Amine | Carbamate | m/z (M + H)⁺ | ¹H NMR (400 MHz, DMSO-d6) |
| I-59^{c} | AJ | AB | 511.7^{b} | 8.99 (s, 1 H), 8.68-8.76 (m, 1 H), 8.55 (t, J = 6.00 Hz, 1 |
| | | | | H), 8.30-8.39 (m, 2 H), 8.21 (d, J = 8.20 Hz, 1 H), 7.36- |
| | | | | 7.46 (m, 4 H), 7.30 (t, J = 7.60 Hz, 1 H), 6.88-7.01 (m, 2 |
| | | | | H), 5.99-6.10 (m, 1 H), 5.74-5.82 (m, 1 H), 5.11 (d, |
| | | | | J = 3.60 Hz, 1 H), 5.06 (dd, J = 13.20, 4.25 Hz, 1 H), 4.33- |
| | | | | 4.49 (m, 4 H), 4.18-4.30 (m, 1 H), 3.60-3.76 (m, 2 |
| | | | | H), 3.36-3.49 (m, 1 H), 3.16-3.32 (m, 5 H), 2.83-3.03 |
| | | | | (m, 4 H), 2.58-2.70 (m, 1 H), 2.45 (s, 3 H), 2.33-2.41 |
| | | | | (m, 1 H), 1.87-2.07 (m, 4 H), 1.59-1.78 (m, 6 H), 1.29- |
| | | | | 1.57 (m, 9H), 0.94 (s, 9 H) |
| I-62 | AO | AB | 1022.1 | 14.41-14.29 (m, 1H), 8.98 (s, 1H), 8.70 (s, 1H), 8.60 (t, |
| | | | | J = 6.0 Hz, 1H), 8.53 (s, 1H), 8.43 (s, 2H), 8.20 (d, J = |
| | | | | 7.6 Hz, 1H), 7.45-7.35 (m, 4H), 7.32-7.26 (m, 1H), |
| | | | | 6.99-6.92 (m, 2H), 6.50-6.44 (m, 1H), 6.28 (d, J = 9.6 |
| | | | | Hz, 1H), 6.03 (d, J = 6.4 Hz, 1H), 5.10-5.02 (m, 1H), |
| | | | | 4.42 (t, J = 8.0 Hz, 2H), 4.38-4.30 (m, 2H), 4.27-4.18 |
| | | | | (m, 2H), 3.91-3.79 (m, 2H), 3.65 (s, 2H), 3.43 (s, 3H), |
| | | | | 3.09 (s, 2H), 2.94 (d, J = 4.8 Hz, 3H), 2.89-2.78 (m, 2H), |
| | | | | 2.44 (s, 3H), 2.14 (d, J = 10.0 Hz, 2H), 2.10-1.98 (m, |
| | | | | 3H), 1.95-1.82 (m, 3H), 1.81-1.70 (m, 2H), 1.54 (d, J = |
| | | | | 6.4 Hz, 5H), 1.51-1.41 (m, 2H), 0.95-0.90 (m, 9H) |
| I-63 | AO | AB | 511.7^{b} | 14.40-14.24 (m, 1H), 12.52-12.36 (m, 1H), 8.99 (s, |
| | | | | 1H), 8.72 (s, 1H), 8.56 (t, J = 6.0 Hz, 1H), 8.34 (s, 2H), |
| | | | | 8.22 (dd, J = 1.6 Hz, J = 8.4 Hz, 1H), 7.40 (s, 4H), 7.33- |
| | | | | 7.27 (m, 1H), 7.00-6.93 (m, 2H), 6.04 (q, J = 6.0 Hz, |
| | | | | 1H), 5.59 (d, J = 9.6 Hz, 1H), 5.13 (d, J = 1.6 Hz, 1H), |
| | | | | 5.09-5.02 (m, 1H), 4.46-4.38 (m, 2H), 4.37-4.31 (m, |
| | | | | 2H), 4.27-4.20 (m, 1H), 3.65 (s, 2H), 3.59 (d, J = 7.6 |
| | | | | Hz, 1H), 3.54-3.49 (m, 2H), 3.45 (d, J = 7.6 Hz, 2H), |
| | | | | 2.98-2.86 (m, 4H), 2.47 (dd, J = 1.6 Hz, J = 3.6 Hz, 2H), |
| | | | | 2.45 (s, 3H), 2.26-2.20 (m, 3H), 2.07-2.01 (m, 1H), |
| | | | | 1.95-1.82 (m, 3H), 1.74-1.64 (m, 4H), 1.61 (s, 2H), |
| | | | | 1.54 (d, J = 6.4 Hz, 3H), 1.45-1.37 (m, 2H), 1.31-1.22 |
| | | | | (m, 2H), 0.93 (s, 9H) |
| I-64 | EK | AB | 499.2^{b} | 14.71 (s, 1H), 8.98 (s, 1H), 8.55 (t, J = 6.0 Hz, 1H), 8.33 |
| | | | | (s, 2H), 7.94 (d, J = 8.0 Hz, 1H), 7.40 (s, 5H), 7.31 (s, |
| | | | | 1H), 7.21 (t, J = 7.6 Hz, 1H), 6.85 (d, J = 8.0 Hz, 2H), |
| | | | | 5.68-5.61 (m, 1H), 5.11 (d, J = 3.6 Hz, 1H), 4.76-4.66 |
| | | | | (m, 2H), 4.45-4.36 (m, 2H), 4.36-4.30 (m, 2H), 4.27- |
| | | | | 4.17 (m, 2H), 3.89-3.81 (m, 2H), 3.79-3.69 (m, 2H), |
| | | | | 3.66-3.58 (m, 3H), 3.30-3.26 (m, 2H), 3.26-3.21 (m, |
| | | | | 1H), 3.18-3.08 (m, 1H), 2.95 (d, J = 3.2 Hz, 1H), 2.85 |
| | | | | (d, J = 8.0 Hz, 2H), 2.79-2.71 (m, 1H), 2.44 (s, 3H), |
| | | | | 2.38 (s, 1H), 2.25 (d, J = 6.0 Hz, 2H), 2.07-2.00 (m, 1H), |
| | | | | 1.95-1.86 (m, 3H), 1.77-1.66 (m, 4H), 1.58 (d, J = 10.8 |
| | | | | Hz, 2H), 0.92 (s, 9H) |
| I-65^{d} | AB | EL | 1023.3 | 14.99-14.35 (m, 1H), 8.98 (s, 1H), 8.56 (t, J = 6.0 Hz, |
| | | | | 1H), 8.33 (s, 2H), 7.94 (d, J = 8.0 Hz, 1H), 7.64 (d, J = |
| | | | | 9.2 Hz, 1H), 7.44-7.35 (m, 5H), 7.31 (s, 1H), 7.24-7.19 |
| | | | | (m, 1H), 6.89-6.82 (m, 2H), 5.13 (d, J = 3.6 Hz, 1H), |
| | | | | 4.74-4.68 (m, 2H), 4.52 (d, J = 9.2 Hz, 1H), 4.41 (s, 2H), |
| | | | | 4.35 (s, 1H), 4.24-4.15 (m, 2H), 3.66 (s, 2H), 3.63-3.57 |
| | | | | (m, 1H), 3.31-3.27 (m, 2H), 3.27-3.21 (m, 1H), 3.19- |
| | | | | 3.09 (m, 2H), 2.95 (d, J = 2.8 Hz, 1H), 2.93-2.86 (m, |
| | | | | 2H), 2.75 (dd, J = 10.8, 12.9 Hz, 1H), 2.44 (s, 3H), 2.39- |
| | | | | 2.34 (m, 1H), 2.20 (d, J = 7.2 Hz, 3H), 2.05-1.99 (m, |
| | | | | 1H), 1.96-1.85 (m, 3H), 1.78 (d, J = 7.6 Hz, 1H), 1.72- |
| | | | | 1.60 (m, 6H), 1.57 (d, J = 9.6 Hz, 3H), 1.30-1.14 (m, |
| | | | | 4H), 0.92 (s, 9H) |
| I-66^{d} | AB | EL | 512.4^{b} | 14.72 (s, 1H), 8.99 (s, 1H), 8.56 (t, J = 6.0 Hz, 1H), 8.37- |
| | | | | 8.30 (m, 2H), 7.95 (d, J = 7.2 Hz, 1H), 7.63 (d, J = 9.2 |
| | | | | Hz, 1H), 7.47-7.36 (m, 5H), 7.32 (s, 1H), 7.26-7.18 (m, |
| | | | | 1H), 6.91-6.81 (m, 2H), 5.14 (d, J = 3.6 Hz, 1H), 4.78- |
| | | | | 4.66 (m, 2H), 4.53 (d, J = 9.2 Hz, 1H), 4.47-4.39 (m, |
| | | | | 2H), 4.36 (d, J = 1.6 Hz, 1H), 4.26-4.16 (m, 2H), 3.74- |
| | | | | 3.64 (m, 2H), 3.62 (dd, J = 2.8, 10.8 Hz, 1H), 3.31-3.27 |
| | | | | (m, 2H), 3.25 (s, 1H), 3.15 (d, J = 8.4 Hz, 2H), 3.00- |
| | | | | 2.94 (m, 1H), 2.91 (d, J = 10.4 Hz, 2H), 2.77 (d, J = 13.2 |
| | | | | Hz, 1H), 2.45 (s, 3H), 2.40-2.35 (m, 1H), 2.25-2.15 (m, |
| | | | | 3H), 2.08-1.99 (m, 1H), 1.91 (dt, J = 4.4, 8.4 Hz, 1H), |
| | | | | 1.81 (dd, J = 9.2, 12.0 Hz, 4H), 1.74-1.61 (m, 5H), 1.58 |
| | | | | (d, J = 10.4 Hz, 3H), 1.31-1.15 (m, 4H), 0.93 (s, 9H). |
| I-67^{c} | ED | AB | 1024.3 | 8.99 (s, 1H), 8.55 (s, 1H), 8.35 (s, 2H), 8.18 (s, 1H), 7.94 |
| | | | | (d, J = 8.0 Hz, 1H), 7.40 (s, 5H), 7.31 (s, 1H), 7.24-7.17 |
| | | | | (m, 1H), 6.89-6.82 (m, 2H), 5.79 (d, J = 8.8 Hz, 1H), |
| | | | | 5.16-5.04 (m, 1H), 4.76-4.66 (m, 2H), 4.47-4.31 (m, |
| | | | | 4H), 4.25 (d, J = 5.6 Hz, 1H), 4.22-4.17 (m, 1H), 3.71- |
| | | | | 3.59 (m, 3H), 3.25 (s, 2H), 3.20 (d, J = 5.2 Hz, 2H), 3.14- |
| | | | | 3.09 (m, 1H), 2.95 (d, J = 3.2 Hz, 1H), 2.89 (d, J = 10.4 |
| | | | | Hz, 2H), 2.75 (dd, J = 10.8 Hz, J = 12.8 Hz, 1H), 2.71- |
| | | | | 2.64 (m, 1H), 2.54 (s, 1H), 2.46-2.44 (m, 3H), 2.39 (s, |
| | | | | 1H), 2.35-2.31 (m, 1H), 2.06-1.87 (m, 4H), 1.79-1.67 |
| | | | | (m, 4H), 1.61 (d, J = 10.4 Hz, 2H), 1.57-1.45 (m, 4H), |
| | | | | 1.38 (s, 2H), 0.93 (s, 9H) |
| I-68 | EE | AB | 1052.3 | 8.98 (s, 1H), 8.55 (t, J = 6.0 Hz, 1H), 8.34 (s, 2H), 8.21 |
| | | | | (s, 1H), 7.94 (d, J = 8.0 Hz, 1H), 7.40 (s, 5H), 7.31 (s, |
| | | | | 1H), 7.25-7.16 (m, 1H), 6.91-6.80 (m, 2H), 5.73 (d, J = |
| | | | | 9.2 Hz, 1H), 4.76-4.64 (m, 2H), 4.48-4.30 (m, 4H), |
| | | | | 4.29-4.15 (m, 2H), 3.74-3.57 (m, 4H), 3.32 (s, 1H), |
| | | | | 3.25 (s, 1H), 3.23 (s, 1H), 3.17-3.07 (m, 2H), 2.97 (d, J = |
| | | | | 9.6 Hz, 3H), 2.75 (dd, J = 10.8 Hz, J = 12.8 Hz, 1H), |
| | | | | 2.54 (s, 1H), 2.46-2.41 (m, 4H), 2.36-2.23 (m, 4H), |
| | | | | 2.12-1.99 (m, 1H), 1.94-1.86 (m, 1H), 1.71 (s, 4H), |
| | | | | 1.65-1.53 (m, 4H), 1.46-1.32 (m, 4H), 1.22 (s, 2H), |
| | | | | 1.07 (t, J = 11.6 Hz, 2H), 0.94 (s, 9H) |
| I-69 | EF | AB | 1024.6 | 8.98 (s, 1H), 8.55 (t, J = 5.8 Hz, 1H), 8.33 (s, 2H), 8.17 |
| | | | | (s, 1H), 7.94 (d, J = 7.8 Hz, 1H), 7.39 (s, 5H), 7.31 (s, |
| | | | | 1H), 7.24-7.18 (m, 1H), 6.89-6.82 (m, 2H), 5.59 (br d, |
| | | | | J = 9.4 Hz, 1H), 5.12 (br d, J = 6.0 Hz, 1H), 4.76-4.67 |
| | | | | (m, 2H), 4.45-4.31 (m, 4H), 4.27-4.17 (m, 2H), 3.65- |
| | | | | 3.62 (m, 2H), 3.61-3.57 (m, 2H), 3.52 (br dd, J = 3.8, |
| | | | | 7.9 Hz, 3H), 3.17-3.09 (m, 4H), 3.00-2.90 (m, 4H), |
| | | | | 2.79-2.71 (m, 1H), 2.44 (s, 3H), 2.30-2.25 (m, 2H), |
| | | | | 2.08-2.00 (m, 1H), 1.94-1.82 (m, 3H), 1.77-1.66 (m, |
| | | | | 4H), 1.63-1.54 (m, 2H), 1.47-1.36 (m, 2H), 1.31-1.23 |
| | | | | (m, 2H), 0.93 (s, 9H) |
| I-70^{d} | AB | EM | 1023.4 | 8.98 (s, 1H), 8.55 (t, J = 5.6 Hz, 1H), 8.35 (s, 2H), 8.14 |
| | | | | (s, 1H), 7.94 (d, J = 8.0 Hz, 1H), 7.70 (d, J = 9.6 Hz, 1H), |
| | | | | 7.43-7.37 (m, 5H), 7.31 (s, 1H), 7.25-7.17 (m, 1H), |
| | | | | 6.91-6.81 (m, 2H), 5.12 (s, 1H), 4.75-4.67 (m, 2H), |
| | | | | 4.50 (d, J = 9.2 Hz, 1H), 4.46-4.39 (m, 2H), 4.35 (s, 1H), |
| | | | | 4.25-4.18 (m, 2H), 3.65-3.59 (m, 3H), 3.28 (s, 1H), |
| | | | | 3.25 (s, 1H), 3.17-3.09 (m, 2H), 2.98-2.91 (m, 3H), |
| | | | | 2.79-2.72 (m, 2H), 2.44 (s, 3H), 2.02-1.99 (m, 1H), |
| | | | | 1.93-1.81 (m, 4H), 1.73-1.24 (m, 16H), 0.95-0.90 (m, 9H) |
| I-71^{d} | AB | EM | 1023.4 | 8.98 (s, 1H), 8.56 (t, J = 5.6 Hz, 1H), 8.35 (s, 2H), 8.18 |
| | | | | (d, J = 5.6 Hz, 1H), 7.94 (d, J = 8.0 Hz, 1H), 7.70 (d, J = |
| | | | | 9.2 Hz, 1H), 7.44-7.36 (m, 5H), 7.31 (s, 1H), 7.24-7.18 |
| | | | | (m, 1H), 6.89-6.83 (m, 2H), 5.33-4.99 (m, 1H), 4.76- |
| | | | | 4.67 (m, 2H), 4.50 (d, J = 9.2 Hz, 1H), 4.46-4.39 (m, |
| | | | | 2H), 4.35 (s, 1H), 4.26-4.16 (m, 2H), 3.69-3.57 (m, |
| | | | | 4H), 3.28-3.22 (m, 2H), 3.16-3.09 (m, 1H), 3.00-2.88 |
| | | | | (m, 3H), 2.80-2.71 (m, 1H), 2.46-2.43 (m, 3H), 2.41- |
| | | | | 2.35 (m, 1H), 2.29 (d, J = 4.4 Hz, 1H), 2.07-1.96 (m, |
| | | | | 2H), 1.90 (ddd, J = 4.8, 8.4, 12.8 Hz, 1H), 1.81-1.59 (m, |
| | | | | 8H), 1.57-1.40 (m, 6H), 1.33-1.21 (m, 3H), 0.92 (s, 8H) |
| I-72 | ES | AB | 997.3 | 8.98 (s, 1H), 8.55 (t, J = 6.0 Hz, 1H), 7.96-7.87 (m, 2H), |
| | | | | 7.43-7.35 (m, 5H), 7.29 (s, 1H), 7.26-7.17 (m, 1H), |
| | | | | 6.89-6.83 (m, 2H), 6.14 (d, J = 6.0 Hz, 1H), 5.67 (d, J = |
| | | | | 9.2 Hz, 1H), 5.12 (d, J = 3.6 Hz, 1H), 4.70 (d, J = 13.2 |
| | | | | Hz, 2H), 4.45-4.37 (m, 2H), 4.36-4.30 (m, 2H), 4.26- |
| | | | | 4.20 (m, 1H), 4.14 (d, J = 12.4 Hz, 1H), 3.91-3.81 (m, |
| | | | | 2H), 3.80-3.71 (m, 2H), 3.67-3.51 (m, 7H), 3.27-3.21 |
| | | | | (m, 2H), 3.03 (d, J = 9.6 Hz, 1H), 2.97-2.88 (m, 1H), |
| | | | | 2.69-2.59 (m, 2H), 2.52 (d, J = 2.0 Hz, 1H), 2.44 (s, 3H), |
| | | | | 2.28 (s, 5H), 2.06-1.86 (m, 4H), 0.92 (s, 9H) |
| I-73 | FJ | EX | 920.4 | 14.41-14.21 (m, 1H), 12.60-12.29 (m, 1H), 8.71 (s, |
| | | | | 1H), 8.55 (t, J = 6.0 Hz, 1H), 8.33 (s, 2H), 8.23-8.19 (m, |
| | | | | 1H), 7.40-7.35 (m, 2H), 7.33-7.27 (m, 3H), 6.99-6.94 |
| | | | | (m, 2H), 6.08-6.01 (m, 1H), 5.65 (d, J = 9.2 Hz, 1H), |
| | | | | 5.12 (d, J = 3.6 Hz, 1H), 5.09-5.01 (m, 1H), 4.43-4.36 |
| | | | | (m, 2H), 4.35-4.30 (m, 2H), 4.23-4.15 (m, 1H), 4.13 |
| | | | | (s, 1H), 3.90-3.86 (m, 1H), 3.85-3.81 (m, 1H), 3.78- |
| | | | | 3.74 (m, 1H), 3.74-3.70 (m, 1H), 3.64 (s, 2H), 3.46- |
| | | | | 3.37 (m, 2H), 3.01-2.89 (m, 2H), 2.85 (d, J = 10.4 Hz, |
| | | | | 2H), 2.59-2.52 (m, 2H), 2.38-2.32 (m, 1H), 2.29-2.22 |
| | | | | (m, 2H), 2.06-1.99 (m, 1H), 1.96-1.90 (m, 2H), 1.89- |
| | | | | 1.84 (m, 1H), 1.84-1.65 (m, 5H), 1.64-1.57 (m, 2H), |
| | | | | 1.54 (d, J = 6.4 Hz, 3H), 0.92 (s, 8H) |

^{a}Step 1 was run anywhere from 60-100° C. for 12 hr. The Step 2 SFC separation was performed with a variety of solvent conditions to separate the chiral compounds. The absolute stereochemistry of the final compounds was assigned arbitrarily for those that had separate diastereomers.
^{b}LCMS data reported as (M/2 + H)⁺.
^{c}No Step 2 SFC separation of the final compound.
^{d}For Step 1, carbamate Intermediate AB was used for the coupling with the indicated acid. The coupling was performed with EDCI, HOAt, DIEA, DMSO at rt for 2 hrs.

TABLE 6A
Compounds synthesized via Method 4, using the corresponding amines and carbamates for the coupling.

| | | | LCMS (ESI+) | |
|---|---|---|---|---|
| I-#^{a} | Amine | Carbamate | m/z (M + H)⁺ | ¹H NMR (400 MHz, DMSO-d6) |
| I-82 | GT | AB | 994.7 | 14.35 (s, 1H) 12.38 (s, 1H) 8.99 (s, 1H) 8.70 (s, 1H) 8.56 (t, |
| | | | | J = 8.0 Hz, 1H) 8.23 (s, 2H) 8.21 (br d, J = 4.0 Hz, 1H) 7.40 |
| | | | | (s, 4H) 7.32-7.28 (m, 1H) 6.98-6.94 (m, 2H) 5.98-5.97 (m, |
| | | | | 1H) 5.67 (d, J = 8.0 Hz, 1H) 5.12 (d, J = 4.0 Hz, 1H) 4.97 (br |
| | | | | dd, J = 12.0, 4.0 Hz, 1H) 4.44-4.38 (m, 2H) 4.35-4.31 (m, |
| | | | | 2H) 4.26-4.21 (m, 1H) 3.90-3.82 (m, 2H) 3.79-3.72 (m, |
| | | | | 2H) 3.64 (br s, 2H) 3.39-3.36 (m, 1H) 3.00 (br s, 4H) 2.97- |
| | | | | 2.87 (m, 3 H) 2.63-2.59 (m, 1H) 2.45 (s, 3H) 2.40-2.33 (m, |
| | | | | 5H) 2.27-2.23 (m, 2H) 2.03-2.01 (m, 1H) 1.98-1.90 (m, 3H) |
| | | | | 1.53 (d, J = 4.0 Hz, 3H) 0.93 (s, 9H) |
| I-96^{b, c} | GV | GW | 500.0 | 8.99 (s, 1H), 8.54 (t, J = 6.0 Hz, 1H), 8.27 (s, 2H), 7.89 (d, J = |
| | | | | 7.6 Hz, 1H), 7.50 (s, 1H), 7.40 (s, 4H), 7.30 (s, 1H), 7.27- |
| | | | | 7.22 (m, 1H), 6.92-6.85 (m, 2H), 5.06 (d, J = 3.6 Hz, 1H), |
| | | | | 4.67-4.58 (m, 3H), 4.47-4.41 (m, 1H), 4.40-4.33 (m, 2H), |
| | | | | 4.28 (d, J = 5.6 Hz, 1H), 4.25-4.20 (m, 1H), 4.10-3.75 (m, |
| | | | | 5H), 3.68-3.59 (m, 3H), 3.30-3.24 (m, 3H), 3.15-3.04 (m, |
| | | | | 4H), 3.04-2.95 (m, 2H), 2.77-2.67 (m, 2H), 2.45 (s, 4H), |
| | | | | 2.42-2.33 (m, 4H), 2.25-2.10 (m, 2H), 2.03-1.96 (m, 1H), |
| | | | | 1.92-1.86 (m, 1H), 0.98 (s, 9H) |
| I-111 | EK | HI | 923.6 | 8.53 (t, J = 6.0 Hz, 1H), 8.35 (s, 2H), 7.91 (d, J = 8.0 Hz, |
| | | | | 1H), 7.45 (s, 1H), 7.39-7.35 (m, 2H), 7.33-7.29 (m, 3H), |
| | | | | 7.23 (t, J = 7.6 Hz, 1H), 6.90-6.85 (m, 2H), 5.78 (d, J = 9.2 |
| | | | | Hz, 1H), 5.12 (d, J = 3.4 Hz, 1H), 4.72 (dd, J = 4.8, 8.0 Hz, |
| | | | | 2H), 4.41-4.31 (m, 4H), 4.23-4.15 (m, 2H), 4.13 (s, 1H), |
| | | | | 3.97-3.72 (m, 5H), 3.69-3.54 (m, 4H), 3.30-3.23 (m, 5H), |
| | | | | 3.20-3.11 (m, 2H), 3.02-2.94 (m, 1H), 2.84-2.71 (m, 2H), |
| | | | | 2.54 (s, 1H), 2.46-2.44 (m, 1H), 2.29 (d, J = 2.0 Hz, 1H), |
| | | | | 2.07-1.68 (m, 7H), 0.92 (s, 9H) |
| I-119 | HT | HU | 950.0 | 14.53-14.22 (m, 1H), 8.71 (s, 1H), 8.46-8.39 (m, 2H), 8.34 |
| | | | | (s, 1H), 8.21 (d, J = 7.3 Hz, 1H), 7.38-7.27 (m, 2H), 7.03 |
| | | | | (s, 1H), 6.99-6.90 (m, 3H), 6.08-6.01 (m, 1H), 5.66 (d, J = |
| | | | | 9.2 Hz, 1H), 5.10-5.02 (m, 1H), 4.43 (t, J = 8.0 Hz, 1H), |
| | | | | 4.36-4.21 (m, 3H), 4.17-4.08 (m, 2H), 3.91-3.87 (m, 1H), |
| | | | | 3.86-3.74 (m, 6H), 3.72 (d, J = 8.6 Hz, 1H), 3.63 (s, 2H), |
| | | | | 3.49 (s, 2H), 2.99-2.80 (m, 4H), 2.59-2.53 (m, 2H), 2.41- |
| | | | | 2.31 (m, 1H), 2.28-2.22 (m, 1H), 2.11-1.97 (m, 3H), 1.96- |
| | | | | 1.85 (m, 3H), 1.78-1.66 (m, 3H), 1.65-1.59 (m, 1H), 1.54 |
| | | | | (d, J = 6.4 Hz, 3H), 0.91 (s, 9H) |
| I-120 | HT | HV | 938.3 | 12.45 (s, 1H), 8.71 (s, 1H), 8.73-8.67 (m, 1H), 8.57 (t, J = |
| | | | | 6.0 Hz, 1H), 8.34 (s, 2H), 8.24-8.19 (m, 1H), 7.48 (t, J = 8.0 |
| | | | | Hz, 1H), 7.34-7.27 (m, 2H), 7.21-7.13 (m, 1H), 7.00-6.93 |
| | | | | (m, 2H), 6.08-6.01 (m, 1H), 5.71 (d, J = 9.2 Hz, 1H), 5.20- |
| | | | | 4.98 (m, 2H), 4.43-4.30 (m, 4H), 4.29-4.19 (m, 2H), 3.93- |
| | | | | 3.88 (m, 1H), 3.87-3.78 (m, 3H), 3.75 (d, J = 8.4 Hz, 1H), |
| | | | | 3.64 (s, 4H), 3.04 (d, J = 9.6 Hz, 2H), 2.99-2.89 (m, 3H), |
| | | | | 2.38-2.29 (m, 2H), 2.13-2.03 (m, 3H), 1.91-1.77 (m, 3H), |
| | | | | 1.72-1.61 (m, 2H), 1.55 (d, J = 6.4 Hz, 3H), 0.91 (s, 9H) |
| I-121 | HT | HJ | 934.5 | 14.43-14.22 (m, 1H), 8.71 (s, 1H), 8.51 (t, J = 6.0 Hz, 1H), |
| | | | | 8.34 (s, 2H), 8.23-8.19 (m, 1H), 7.33-7.24 (m, 5H), 6.99- |
| | | | | 6.94 (m, 2H), 6.07-6.01 (m, 1H), 5.64 (d, J = 9.6 Hz, 1H), |
| | | | | 5.13-5.01 (m, 2H), 4.43-4.29 (m, 4H), 4.20-4.12 (m, 1H), |
| | | | | 3.91-3.80 (m, 2H), 3.79-3.70 (m, 2H), 3.63 (s, 2H), 3.45- |
| | | | | 3.37 (m, 1H), 3.01-2.90 (m, 2H), 2.85 (d, J = 10.4 Hz, 2H), |
| | | | | 2.53 (s, 1H), 2.42-2.32 (m, 3H), 2.29-2.22 (m, 2H), 2.03 |
| | | | | (s, 3H), 1.95-1.83 (m, 4H), 1.77-1.68 (m, 4H), 1.65-1.57 |
| | | | | (m, 2H), 1.54 (d, J = 6.6 Hz, 3H), 0.94-0.89 (m, 9H) |
| I-127 | FJ | IU | 1021.7 | 12.44 (s, 1H), 8.72-8.69 (m, 1H), 8.45-8.40 (m, 1H), 8.33 |
| | | | | (s, 2H), 8.20 (d, J = 7.6 Hz, 1H), 8.15 (s, 1H), 7.36-7.26 (m, |
| | | | | 2H), 7.03-6.99 (m, 1H), 6.99-6.91 (m, 3H), 6.11-5.98 (m, |
| | | | | 1H), 5.73-5.60 (m, 1H), 5.19-4.99 (m, 2H), 4.50-4.39 (m, |
| | | | | 1H), 4.37-4.27 (m, 2H), 4.24 (d, J = 6.4 Hz, 1H), 4.17 (d, J = |
| | | | | 5.0 Hz, 1H), 4.14 (s, 1H), 4.05 (s, 2H), 3.89 (d, J = 8.0 Hz, |
| | | | | 1H), 3.83 (d, J = 8.0 Hz, 1H), 3.80-3.75 (m, 1H), 3.72 (d, J = |
| | | | | 8.8 Hz, 1H), 3.63 (s, 2H), 3.10 (s, 1H), 2.96-2.82 (m, 5H), |
| | | | | 2.77-2.69 (m, 2H), 2.42 (s, 6H), 2.30-2.22 (m, 2H), 1.95 |
| | | | | (d, J = 5.6 Hz, 7H), 1.81 (s, 2H), 1.75-1.67 (m, 2H), 1.59 |
| | | | | (d, J = 12.0 Hz, 2H), 1.54 (d, J = 6.4 Hz, 3H), 0.90 (s, 9H) |
| I-128 | HT | HZ | 1008.8 | 14.33 (s, 1H), 12.59-12.29 (m, 1H), 8.71 (s, 1H), 8.44-8.37 |
| | | | | (m, 1H), 8.33 (s, 2H), 8.21 (dd, J = 1.2, 8.4 Hz, 1H), 7.35- |
| | | | | 7.26 (m, 2H), 7.04-6.90 (m, 4H), 6.04 (d, J = 6.4 Hz, 1H), |
| | | | | 5.66 (d, J = 9.6 Hz, 1H), 5.12 (d, J = 3.5 Hz, 1H), 5.08-4.99 |
| | | | | (m, 1H), 4.43 (t, J = 8.0 Hz, 1H), 4.37-4.26 (m, 2H), 4.23 |
| | | | | (d, J = 6.4 Hz, 1H), 4.18-4.09 (m, 2H), 4.05 (t, J = 6.0 Hz, |
| | | | | 2H), 3.91-3.80 (m, 2H), 3.79-3.69 (m, 2H), 3.63 (s, 2H), |
| | | | | 3.49 (t, J = 6.4 Hz, 2H), 3.45-3.38 (m, 2H), 3.27-3.24 (m, |
| | | | | 3H), 3.01-2.89 (m, 2H), 2.85 (d, J = 10.4 Hz, 2H), 2.29- |
| | | | | 2.20 (m, 2H), 2.09-1.99 (m, 2H), 1.98-1.85 (m, 5H), 1.79- |
| | | | | 1.66 (m, 4H), 1.65-1.56 (m, 2H), 1.54 (d, J = 6.4 Hz, 3H), |
| | | | | 0.98-0.84 (m, 9H) |
| I-136 | IY | HI | 934.5 | 12.42 (s, 1H), 8.71 (s, 1H), 8.54 (t, J = 5.6 Hz, 1H), 8.30 (s, |
| | | | | 2H), 8.21 (d, J = 7.6 Hz, 1H), 7.43-7.26 (m, 5H), 7.07-6.87 |
| | | | | (m, 2H), 6.07 (dd, J = 3.6, 9.4 Hz, 1H), 5.66 (d, J = 9.1 Hz, |
| | | | | 1H), 5.11 (s, 1H), 5.01-4.93 (m, 1H), 4.47-4.27 (m, 5H), |
| | | | | 4.24-4.09 (m, 3H), 3.92-3.81 (m, 3H), 3.79-3.69 (m, 3H), |
| | | | | 3.63 (s, 3H), 2.41 (d, J = 8.0 Hz, 2H), 2.21-2.09 (m, 2H), |
| | | | | 2.06-1.94 (m, 4H), 1.89 (dd, J = 4.4, 7.6 Hz, 4H), 1.79- |
| | | | | 1.53 (m, 5H), 1.02 (t, J = 7.2 Hz, 3H), 0.92 (s, 9H) |
| I-138^{d} | FJ | JT | 921.7 | 12.45 (s, 1H) 8.71 (s, 1H) 8.53 (br t, J = 5.6 Hz, 1H) 8.34 (s, |
| | | | | 2H) 8.24-8.19 (m, 1H) 7.41-7.37 (m, 2H) 7.33-7.28 (m, |
| | | | | 3H) 6.99-6.93 (m, 2H) 6.05 (br d, J = 6.4 Hz, 1H) 5.11- |
| | | | | 5.03 (m, 2H) 4.63 (s, 1H) 4.42 (t, J = 8.0 Hz, 1H) 4.38-4.31 |
| | | | | (m, 2H) 4.26-4.17 (m, 1H) 4.13 (s, 1H) 4.08-3.74 (m, 5H) |
| | | | | 3.72-3.58 (m, 3H) 3.12-3.01 (m, 2H) 2.97-2.85 (m, 3H) |
| | | | | 2.55 (br s, 2H) 2.39 (br d, J = 2.4 Hz, 2H) 2.14-2.08 (m, 1H) |
| | | | | 2.02-1.96 (m, 1H) 1.90-1.79 (m, 3H) 1.71-1.61 (m, 2H) |
| | | | | 1.55 (d, J = 6.4 Hz, 3H) 0.97 (s, 9H) |
| I-139 | FJ | JH | 934.5 | 14.33 (s, 1 H), 12.37-12.54 (m, 1 H), 8.71 (s, 1 H), 8.59 (t, |
| | | | | J = 6.00 Hz, 1 H), 8.33 (s, 2 H), 8.21 (dd, J = 8.40, 1.20 Hz, 1 |
| | | | | H), 7.36-7.47 (m, 2 H), 7.25-7.35 (m, 3 H), 6.91-7.02 (m, |
| | | | | 2 H), 6.04 (q, J = 6.00 Hz, 1 H), 4.99-5.16 (m, 2 H), 4.54 (t, |
| | | | | J = 8.40 Hz, 1 H), 4.35-4.43 (m, 1 H), 4.19-4.27 (m, 1 H), |
| | | | | 4.11-4.17 (m, 1 H), 3.87-3.95 (m, 2 H), 3.70-3.81 (m, 2 |
| | | | | H), 3.63 dd, J = 11.60, 2.80 Hz, 1 H), 2.88-3.05 (m, 3 H), |
| | | | | 2.83 (d, J = 9.60 Hz, 2 H), 2.65-2.76 (m, 1 H), 2.31-2.43 |
| | | | | (m, 2 H), 2.23 (dd, J = 12.80, 7.20 Hz, 3 H), 2.16 (s, 3 H), 2.05 |
| | | | | (ddd, J = 13.60, 9.20, 4.40 Hz, 1H), 1.87-1.95 (m, 2 H), 1.66- |
| | | | | 1.80 (m, 5 H), 1.47-1.63 (m, 6 H), 0.91 (s, 9 H) |
| I-140 | FJ | JV | 978.7 | 14.33 (s, 1 H), 12.31-12.60 (m, 1 H), 8.71 (s, 1 H), 8.60 (t, |
| | | | | J = 6.20 Hz, 1 H), 8.33 (s, 2 H), 8.21 (dd, J = 8.40, 1.60 Hz, 1 |
| | | | | H), 7.36-7.48 (m, 2 H), 7.24-7.35 (m, 3 H), 6.91-7.02 (m, |
| | | | | 2 H), 5.89-6.14 (m, 1 H), 4.99-5.18 (m, 2 H), 4.48-4.58 |
| | | | | (m, 1 H), 4.34-4.44 (m, 1 H), 4.19-4.31 (m, 1 H), 4.05- |
| | | | | 4.18 (m, 1 H), 3.83-3.99 (m, 3 H), 3.68-3.80 (m, 2 H), 3.61 |
| | | | | (dd, J = 11.60, 3.20 Hz, 1 H), 3.37-3.44 (m, 2 H), 3.24 (s, 3 |
| | | | | H), 3.08-3.20 (m, 2 H), 2.89-3.01 (m, 2 H), 2.84 (d, J = 9.60 |
| | | | | Hz, 2 H), 2.58-2.72 (m, 2 H), 2.31-2.47 (m, 3 H), 2.17- |
| | | | | 2.30 (m, 3 H), 2.05 (ddd, J = 13.60, 9.20, 4.40 Hz, 1 H), 1.93 |
| | | | | (dd, J = 10.40, 8.40 Hz, 2 H), 1.67-1.77 (m, 4 H), 1.52-1.63 |
| | | | | (m, 5 H), 0.91 (s, 9 H) |
| I-141 | FJ | JZ | 992.2 | 8.40 (s, 1H), 7.48-7.23 (m, 5H), 7.18-7.13 (m, 2H), 6.77- |
| | | | | 6.73 (m, 2H), 4.50-4.20 (m, 5H), 4.17-3.92 (m, 4H), 3.78- |
| | | | | 3.69 (m, 2H), 3.41-3.35 (m, 5H), 3.26-3.19 (m, 3H), 2.34 |
| | | | | (d, J = 7.0 Hz, 2H), 2.16-2.09 (m, 3H), 2.04 (s, 1H), 1.96- |
| | | | | 1.87 (m, 1H), 1.03 (d, J = 3.6 Hz, 3H), 0.96 (s, 1H) |
| I-142 | FJ | MI | 1019 | 14.55-14.03 (m, 1H), 12.45 (s, 1H), 8.71 (s, 1H), 8.63 (d, J = |
| | | | | 4.4 Hz, 1H), 8.34 (s, 2H), 8.20 (d, J = 7.2 Hz, 1H), 7.39- |
| | | | | 7.27 (m, 3H), 7.22 (d, J = 7.6 Hz, 1H), 7.02-6.89 (m, 2H), |
| | | | | 6.04 (q, J = 6.8 Hz, 1H), 5.05 (dd, J = 5.6, 8.0 Hz, 1H), 4.77 |
| | | | | (dd, J = 4.0, 7.2 Hz, 1H), 4.48-4.31 (m, 3H), 4.29-4.15 (m, |
| | | | | 2H), 4.03-3.93 (m, 1H), 3.89-3.60 (m, 6H), 3.19-3.11 (m, |
| | | | | 4H), 3.00-2.86 (m, 3H), 2.54 (s, 2H), 2.52 (d, J = 2.0 Hz, |
| | | | | 3H), 2.40-2.34 (m, 2H), 2.08-1.81 (m, 6H), 1.54 (d, J = |
| | | | | 6.4 Hz, 3H), 1.26-1.21 (m, 1H), 0.94 (s, 9H) |
| I-144^{e} | FJ | JB | 994.7 | 0.90 (s, 9 H) 1.53 (d, J = 6.50 Hz, 3 H) 1.60-1.66 (m, 2 H) |
| | | | | 1.73 (d, J = 10.51 Hz, 2 H) 1.85-1.90 (m, 3 H) 1.95-2.03 |
| | | | | (m, 3 H) 2.23-2.30 (m, 2 H) 2.31-2.35 (m, 1 H) 2.36-2.40 |
| | | | | (m, 1 H) 2.64-2.70 (m, 1 H) 2.86-2.94 (m, 4 H) 3.62 (br s, |
| | | | | 3 H) 3.71-3.89 (m, 5 H) 4.15 (s, 1 H) 4.19 (br d, J = 5.63 Hz, |
| | | | | 1 H) 4.26-4.36 (m, 3 H) 4.42 (br t, J = 8.00 Hz, 1 H) 4.68 (s, |
| | | | | 2 H) 5.01-5.13 (m, 2 H) 5.63 (br d, J = 9.51 Hz, 1 H) 6.03 (q, |
| | | | | J = 6.30 Hz, 1 H) 6.91-6.98 (m, 4 H) 7.25-7.36 (m, 2 H) |
| | | | | 8.17-8.23 (m, 1 H) 8.33 (s, 2 H) 8.55-8.58 (m, 1 H) 8.70 |
| | | | | (s, 1 H) 12.43 (br s, 1 H) |
| I-146 | JL | HI | 936.4 | 14.33-14.26 (m, 1H), 12.41 (m, 1H), 8.72 (s, 1H), 8.61- |
| | | | | 8.46 (m, 1H), 8.38-8.25 (m, 2H), 8.14-8.03 (m, 1H), 7.42- |
| | | | | 7.27 (m, 5H), 7.01-6.91 (m, 2H), 6.08-5.96 (m, 1H), 5.70- |
| | | | | 5.56 (m, 1H), 5.16-5.01 (m, 2H), 4.99-4.95 (m, 1H), 4.45- |
| | | | | 4.29 (m, 4H), 4.24-4.09 (m, 2H), 3.96 (td, J = 5.0, 10.4 |
| | | | | Hz, 1H), 3.90-3.81 (m, 3H), 3.79-3.70 (m, 2H), 2.97-2.82 |
| | | | | (m, 5H), 2.29-2.21 (m, 4H), 2.07-1.98 (m, 2H), 1.95-1.86 |
| | | | | (m, 3H), 1.80-1.67 (m, 5H), 1.64-1.54 (m, 2H), 1.29-1.11 |
| | | | | (m, 2H), 0.97-0.86 (m, 9H) |
| I-147 | JL | HI | 936.6 | 14.41 . 14.18 (m, 1H), 12.55-12.35 (m, 1H), 8.69 (s, 1H), |
| | | | | 8.56-8.50 (m, 1H), 8.29 (s, 2H), 8.20 (s, 1H), 8.11 (d, J = |
| | | | | 0.8 Hz, 1H), 7.34 (s, 2H), 7.30 (d, J = 8.4 Hz, 2H), 7.00- |
| | | | | 6.93 (m, 2H), 5.99 (s, 1H), 5.67-5.60 (m, 1H), 5.12-5.09 |
| | | | | (m, 1H), 4.99-4.93 (m, 1H), 4.42-4.28 (m, 4H), 4.20 (d, J = |
| | | | | 6.0 Hz, 1H), 4.12 (s, 1H), 3.99-3.91 (m, 1H), 3.90-3.79 |
| | | | | (m, 3H), 3.78-3.69 (m, 2H), 3.63 (s, 2H), 2.94-2.81 (m, |
| | | | | 3H), 2.29-2.20 (m, 4H), 2.05-1.96 (m, 2H), 1.90 (td, J = |
| | | | | 8.0, 11.6 Hz, 4H), 1.77-1.67 (m, 4H), 1.63-1.54 (m, 2H), |
| | | | | 1.13 (s, 1H), 0.95-0.87 (m, 9H) |
| I-150^{d} | FJ | LA | 935.7 | 12.43 (s, 1H), 8.70 (s, 1H), 8.37-8.32 (m, 3H), 8.20 (d, J = |
| | | | | 8.4 Hz, 1H), 7.41 (d, J = 8.0 Hz, 2H), 7.31-7.24 (m, 3H), |
| | | | | 6.99-6.92 (m, 2H), 6.04 (q, J = 6.4 Hz, 1H), 5.10-4.96 (m, |
| | | | | 2H), 4.92-4.92 (m, 1H), 4.85 (t, J = 7.2 Hz, 1H), 4.61 (s, |
| | | | | 1H), 4.42 (t, J = 9.2 Hz, 1H), 4.26 (s, 1H), 4.11 (s, 1H), 4.05- |
| | | | | 3.67 (m, 5H), 3.64-3.60 (m, 1H), 3.57 (d, J = 4.4 Hz, 1H), |
| | | | | 3.43 (s, 1H), 2.99-2.99 (m, 1H), 2.99-2.89 (m, 2H), 2.85 |
| | | | | (d, J = 10.8 Hz, 2H), 2.54 (s, 1H), 2.51-2.51 (m, 1H), 2.33 |
| | | | | (dd, J = 1.6, 3.6 Hz, 1H), 2.31-2.25 (m, 2H), 1.94 (t, J = 9.6 |
| | | | | Hz, 3H), 1.80-1.68 (m, 5H), 1.59 (d, J = 12.0 Hz, 2H), 1.54 |
| | | | | (d, J = 6.4 Hz, 3H), 1.32 (d, J = 7.2 Hz, 3H), 0.96-0.96 (m, |
| | | | | 1H), 0.96-0.96 (m, 1H), 0.96 (s, 8H) |
| I-151 | FJ | JE | 934.5 | 13.96-14.72 (m, 1 H), 12.32-12.57 (m, 1 H), 8.71 (s, 1 H), |
| | | | | 8.29-8.43 (m, 3 H), 8.21 d, J = 7.20 Hz, 1 H), 7.42 (d, J = 8.20 |
| | | | | Hz, 2 H), 7.22-7.35 (m, 3 H), 6.87-7.01 (m, 2 H), 6.04 (q, |
| | | | | J = 6.40 Hz, 1H), 5.56 (d, J = 9.20 Hz, 1 H), 5.02-5.11 (m, 1 |
| | | | | H), 4.87 (t, J = 7.20 Hz, 1 H), 4.40 (t, J = 8.00 Hz, 1 H), 4.22- |
| | | | | 4.33 (m, 2 H), 4.12 (s, 1 H), 3.79-3.91 (m, 2 H), 3.71-3.79 |
| | | | | (m, 2 H), 3.50-3.62 (m, 4 H), 2.82-2.98 (m, 4 H), 2.31- |
| | | | | 2.45 (m, 2 H), 2.25 (t, J = 8.20 Hz, 2 H), 1.88-2.05 (m, 3 H), |
| | | | | 1.66-1.81 (m, 5 H), 1.50-1.64 (m, 5 H), 1.33 (d, J = 7.20 |
| | | | | Hz, 3 H), 0.92 (s, 9 H) |
| I-159 | KM | HI | 924 | 12.43 (br s, 1H) 8.70 (s, 1H) 8.55 (t, J = 6.0 Hz, 1H) 8.23- |
| | | | | 8.20 (m, 3H) 7.40-7.36 (m, 2H) 7.34-7.28 (m, 3H) 6.99- |
| | | | | 6.94 (m, 2H) 6.00-5.95 (m, 1H) 5.86 (br d, J = 8.8 Hz, 1H) |
| | | | | 5.12 (br s, 1H) 4.96 (br dd, J = 13.6, 5.2 Hz, 1H) 4.44-4.31 |
| | | | | (m, 5H) 4.19 (dd, J = 16.0, 5.4 Hz, 1H) 4.13 (s, 1H) 3.72- |
| | | | | 3.63 (m, 2H) 3.47-3.42 (m, 4H) 3.05-2.79 (m, 3H) 2.63- |
| | | | | 2.54 (m, 4H) 2.34-2.28 (m, 4H) 2.17 (br d, J = 8.0 Hz, 2H) |
| | | | | 2.06-1.99 (m, 1H) 1.88 (dd, J = 12.8, 8.4 Hz, 1H) 1.79 (br |
| | | | | d, J = 11.2 Hz, 2H) 1.66-1.57 (m, 1H) 1.53 (d, J = 6.4 Hz, |
| | | | | 3H) 1.29-1.20 (m, 2H) 0.94 (s, 9H) |
| I-160 | KM | HI | 924.6 | 12.42 (s, 1H) 8.70 (s, 1H) 8.55 (t, J = 6.4 Hz, 1H) 8.24-8.20 |
| | | | | (m, 3H) 7.40-7.35 (m, 2H) 7.33-7.28 (m, 3H) 6.99-6.94 |
| | | | | (m, 2H) 6.00-5.95 (m, 1H) 5.86 (br d, J = 8.8 Hz, 1H) 5.12 |
| | | | | (br d, J = 2.8 Hz, 1H) 4.96 (br dd, J = 13.6, 5.2 Hz, 1H) 4.44- |
| | | | | 4.31 (m, 5H) 4.19 (dd, J = 16.0, 5.4 Hz, 1H) 4.13 (s, 1H) |
| | | | | 3.72-3.63 (m, 2H) 3.47-3.42 (m, 4H) 3.05-2.79 (m, 3H) |
| | | | | 2.63-2.54 (m, 4H) 2.34-2.28 (m, 4H) 2.17 (br d, J = 8.0 |
| | | | | Hz, 2H) 2.06-1.99 (m, 1H) 1.88 (dd, J = 12.8, 8.4 Hz, 1H) |
| | | | | 1.79 (br d, J = 11.2 Hz, 2H) 1.66-1.57 (m, 1H) 1.53 (d, J = |
| | | | | 6.4 Hz, 3H) 1.29-1.20 (m, 2H) 0.94 (s, 9H) |
| I-161^{f} | MR | HI | 909.8 | 14.51-14.21 (m, 1H), 12.39 (s, 1H), 8.69 (s, 1H), 8.53 (t, J = |
| | | | | 6.0 Hz, 1H), 8.25-8.19 (m, 1H), 7.94 (s, 2H), 7.43-7.34 |
| | | | | (m, 2H), 7.33-7.26 (m, 3H), 7.00-6.93 (m, 2H), 5.93 (q, J = |
| | | | | 6.4 Hz, 1H), 5.86 (d, J = 9.2 Hz, 1H), 5.10 (s, 1H), 4.90 |
| | | | | (dd, J = 5.2, 13.2 Hz, 1H), 4.45-4.32 (m, 4H), 4.25-4.08 |
| | | | | (m, 2H), 3.73-3.61 (m, 2H), 3.25 (d, J = 4.4 Hz, 2H), 3.21- |
| | | | | 3.12 (m, 2H), 2.98-2.82 (m, 3H), 2.54 (s, 1H), 2.52 (s, 4H), |
| | | | | 2.34 (dd, J = 1.6, 7.2 Hz, 6H), 2.06-1.96 (m, 2H), 1.88 (ddd, |
| | | | | J = 4.8, 8.4, 12.8 Hz, 1H), 1.66 (dd, J = 7.2, 12.0 Hz, 1H), |
| | | | | 1.50 (d, J = 6.4 Hz, 3H), 0.94 (s, 9H) |
| I-162^{f} | MR | HI | 909.8 | 14.41-14.28 (m, 1H), 12.39 (s, 1H), 8.72-8.65 (m, 1H), |
| | | | | 8.53 (t, J = 6.0 Hz, 1H), 8.24-8.18 (m, 1H), 7.94 (s, 2H), |
| | | | | 7.39-7.35 (m, 2H), 7.33-7.27 (m, 3H), 6.99-6.93 (m, 2H), |
| | | | | 5.99-5.79 (m, 2H), 5.11 (d, J = 3.6 Hz, 1H), 4.94-4.85 (m, |
| | | | | 1H), 4.44-4.33 (m, 4H), 4.22-4.11 (m, 2H), 3.71-3.61 (m, |
| | | | | 2H), 3.27-3.11 (m, 4H), 2.97-2.82 (m, 3H), 2.54 (s, 1H), |
| | | | | 2.52 (d, J = 2.0 Hz, 4H), 2.40-2.30 (m, 6H), 2.08-1.98 (m, |
| | | | | 2H), 1.91-1.84 (m, 1H), 1.72-1.62 (m, 1H), 1.50 (d, J = |
| | | | | 6.4 Hz, 3H), 0.94 (s, 9H) |
| I-163 | LB | HI | 926.8 | 8.54 (t, J = 6 Hz, 1H), 8.24 (s, 2H), 7.95 (d, J = 8 Hz, 1H), |
| | | | | 7.41-7.35 (m, 3H), 7.33-7.30 (m, 3H), 7.24-7.19 (m, 1H), |
| | | | | 6.89-6.83 (t, J = 15.6 Hz, 2H), 5.85 (d, J = 9.2 Hz, 1H), 5.11 |
| | | | | (d, J = 3.6 Hz, 1H), 4.62 (d, J = 12.4 Hz, 2H), 4.44-4.39 (m, 2 |
| | | | | H), 4.37 (d, J = 9.2 Hz, 2H), 4.22-4.16 (m, 2H), 4.13 (s, 1H), |
| | | | | 3.74-3.64 (m, 2H), 3.63-3.73 (m, 1H), 3.47 (d, J = 11.2 Hz, |
| | | | | 2H), 3.12-2.87 (m, 3H), 2.75-2.64 (m, 2H), 2.59 (t, J = 11.6 |
| | | | | Hz, 2H), 2.37-2.26 (m, 6H), 2.17 (d, J = 6.8 Hz, 2H), 2.08- |
| | | | | 1.97 (m, 2H), 1.92-1.86 (m, 1H), 1.79 (d, J = 11.8 Hz, 2H), |
| | | | | 1.66-1.58 (m, 1H), 1.31-1.17 (m, 3H), 0.95 (s, 9H) |
| I-164 | KO | HI | 935.8 | 12.43 (br s, 1H) 8.70 (s, 1H) 8.55 (t, J = 6.0 Hz, 1H) 8.23- |
| | | | | 8.19 (m, 3H) 7.40-7.35 (m, 2H) 7.33-7.27 (m, 3H) 6.99- |
| | | | | 6.92 (m, 2H) 5.97 (br d, J = 6.4 Hz, 1H) 5.77 (br d, J = 9.2 |
| | | | | Hz, 1H) 5.25-5.04 (m, 1H) 4.96 (br d, J = 8.4 Hz, 1H) 4.42- |
| | | | | 4.36 (m, 2H) 4.33 (br d, J = 9.6 Hz, 2H) 4.19 (br dd, J = |
| | | | | 16.0, 5.6 Hz, 1H) 4.14 (s, 1H) 3.93-3.85 (m, 4H) 3.64 (br s, |
| | | | | 2H) 3.42-3.40 (m, 3H) 3.23 (s, 4H) 2.97-2.84 (m, 2H) 2.53 |
| | | | | (d, J = 1.6 Hz, 2H) 2.25 (br d, J = 6.4 Hz, 2H) 2.05-1.98 |
| | | | | (m, 1H) 1.87 (s, 1H) 1.72 (br d, J = 12.0 Hz, 2H) 1.52 (d, J = |
| | | | | 6.4 Hz, 3H) 1.44-1.10 (m, 4H) 0.92 (s, 8H) |
| I-165 | KO | HI | 935.5 | 12.43 (br s, 1H) 8.70 (s, 1H) 8.58-8.53 (m, 1H) 8.35-8.13 |
| | | | | (m, 4H) 7.39-7.36 (m, 2H) 7.33-7.28 (m, 3H) 6.98-6.94 |
| | | | | (m, 2H) 5.99-5.95 (m, 1H) 5.76 (br d, J = 9.2 Hz, 1H) 4.96 |
| | | | | (br dd, J = 12.0, 4.0 Hz, 1H) 4.41-4.35 (m, 2H) 4.33 (br d, |
| | | | | J = 9.6 Hz, 2H) 4.22-4.16 (m, 1H) 4.13 (s, 1H) 3.93-3.58 |
| | | | | (m, 4H) 3.64 (br s, 2H) 3.39-3.38 (m, 3H) 3.21 (s, 4H) 2.97- |
| | | | | 2.85 (m, 2H) 2.53 (br d, J = 2.0 Hz, 2H) 2.24 (br d, J = 6.4 |
| | | | | Hz, 2H) 2.06-1.99 (m, 1H) 1.88 (t, J = 8.4 Hz, 1H) 1.72 (br |
| | | | | d, J = 12.0 Hz, 2H) 1.52 (d, J = 6.4 Hz, 3H) 1.41-1.13 (m, |
| | | | | 4H) 0.92 (s, 9H) |
| I-171 | KY | HI | 947.5 | 14.16-14.49 (m, 1 H), 12.07-12.70 (m, 1 H), 8.65-8.73 |
| | | | | (m, 1 H), 8.45-8.57 (m, 1 H), 8.16-8.25 (m, 1 H), 8.04 (s, |
| | | | | 1 H), 7.26-7.42 (m, 5 H), 6.88-7.05 (m, 2 H), 5.87-6.02 |
| | | | | (m, 1 H), 5.63 (d, J = 9.20 Hz, 1 H), 5.03-5.20 (m, 1 H), 4.87- |
| | | | | 5.01 (m, 1 H), 4.27-4.46 (m, 4 H), 4.09-4.24 (m, 2 H), |
| | | | | 3.56-3.91 (m, 6 H), 3.51 (d, J = 11.6 Hz, 1 H), 3.27 (d, J = 3.20 |
| | | | | Hz, 3 H), 2.77-3.04 (m, 6 H), 2.60-2.76 (m, 2 H), 2.12- |
| | | | | 2.43 (m, 4 H), 1.79-2.10 (m, 5 H), 1.48-1.59 (m, 3 H), 0.87- |
| | | | | 0.96 (m, 9 H) |
| I-172 | KY | HI | 947.5 | 14.06-14.56 (m, 1 H), 12.03-12.76 (m, 1 H), 8.69 (s, 1 H), |
| | | | | 8.51-8.60 (m, 1 H), 8.21 (d, J = 8.00 Hz, 1 H), 8.04 (s, 1 H), |
| | | | | 7.27-7.46 (m, 5 H), 6.91-7.07 (m, 2 H), 5.88-6.03 (m, 1 |
| | | | | H), 4.90-5.07 (m, 1 H), 4.28-4.44 (m, 4 H), 4.14-4.24 (m, |
| | | | | 1 H), 3.65-3.89 (m, 5 H), 3.54-3.63 (m, 4 H), 2.88-3.05 |
| | | | | (m, 5 H), 2.63-2.75 (m, 6 H), 2.11-2.34 (m, 5 H), 1.79- |
| | | | | 2.07 (m, 5 H), 1.43-1.59 (m, 3 H), 0.85-0.95 (m, 9 H) |
| I-173 | LD | HI | 961.7 | 14.56-14.28 (m, 1H), 14.36 (s, 1H), 12.22-12.21 (m, 1H), |
| | | | | 12.40 (s, 1H), 8.69 (s, 1H), 8.54 (t, J = 6.0 Hz, 1H), 8.21 (dd, |
| | | | | J = 1.2, 8.4 Hz, 1H), 7.91 (s, 2H), 7.43-7.34 (m, 2H), 7.33- |
| | | | | 7.21 (m, 3H), 7.02-6.90 (m, 2H), 5.93 (q, J = 6.8 Hz, 1H), |
| | | | | 5.64 (d, J = 9.6 Hz, 1H), 5.11 (d, J = 2.8 Hz, 1H), 4.88 (s, |
| | | | | 1H), 4.46-4.27 (m, 4H), 4.13 (s, 2H), 3.81 (s, 4H), 3.62 (s, |
| | | | | 2H), 3.23 (t, J = 7.2 Hz, 2H), 3.17 (dd, J = 6.0, 8.8 Hz, 1H), |
| | | | | 3.12-3.05 (m, 1H), 3.00-2.90 (m, 1H), 2.89-2.80 (m, 1H), |
| | | | | 2.79-2.72 (m, 1H), 2.69-2.64 (m, 1H), 2.46-2.37 (m, 3H), |
| | | | | 2.36-2.26 (m, 2H), 2.19 (dd, J = 6.4, 10.0 Hz, 2H), 2.03- |
| | | | | 1.94 (m, 3H), 1.82-1.82 (m, 1H), 1.93 (s, 2H), 1.80-1.69 |
| | | | | (m, 2H), 1.50 (d, J = 6.4 Hz, 3H), 0.98-0.85 (m, 9H) |
| I-179 | LL | HI | 925.6 | 12.29 (s, 1H), 8.57-8.48 (m, 2H), 8.19 (d, J = 0.8 Hz, 1H), |
| | | | | 8.10 (d, J = 7.2 Hz, 1H), 7.40-7.33 (m, 2H), 7.33-7.25 (m, |
| | | | | 3H), 6.98-6.91 (m, 2H), 5.63 (d, J = 9.6 Hz, 1H), 4.40-4.29 |
| | | | | (m, 5H), 4.21-4.15 (m, 1H), 4.12 (s, 1H), 3.86-3.84 (m, |
| | | | | 1H), 3.81-3.79 (m, 1H), 3.73 (s, 2H), 3.69 (d, J = 8.8 Hz, |
| | | | | 3H), 3.62 (s, 2H), 3.07 (s, 2H), 2.97 (d, J = 6.4 Hz, 2H), 2.90- |
| | | | | 2.84 (m, 1H), 2.83-2.78 (m, 2H), 2.70 (s, 1H), 2.60 (d, J = |
| | | | | 11.6 Hz, 1H), 2.54 (s, 1H), 2.52 (s, 1H), 2.24-2.18 (m, 2H), |
| | | | | 2.05-1.98 (m, 1H), 1.94-1.81 (m, 5H), 1.76-1.64 (m, 4H), |
| | | | | 1.53 (s, 2H), 1.38 (d, J = 6.4 Hz, 4H), 1.15 (d, J = 6.4 Hz, |
| | | | | 2H), 0.94-0.88 (m, 9H) |
| I-180 | LM | HI | 926.7 | 12.28 (s, 1H), 8.57-8.46 (m, 2H), 8.18 (s, 1H), 8.10 (dd, J = |
| | | | | 1.2, 8.4 Hz, 1H), 7.40-7.33 (m, 2H), 7.32-7.25 (m, 3H), |
| | | | | 7.01-6.91 (m, 2H), 5.63 (d, J = 9.2 Hz, 1H), 4.42-4.38 (m, |
| | | | | 1H), 4.37-4.29 (m, 4H), 4.18 (dd, J = 5.6, 15.6 Hz, 1H), |
| | | | | 4.13-4.11 (m, 1H), 3.85 (d, J = 8.0 Hz, 1H), 3.80 (d, J = 8.0 |
| | | | | Hz, 1H), 3.75-3.68 (m, 4H), 3.62 (s, 2H), 3.08 (d, J = 11.6 |
| | | | | Hz, 2H), 2.97-2.91 (m, 2H), 2.85-2.77 (m, 3H), 2.69 (d, J = |
| | | | | 3.6 Hz, 1H), 2.54 (s, 2H), 2.52 (s, 1H), 2.25 (s, 1H), 2.23- |
| | | | | 2.18 (m, 2H), 2.04-1.98 (m, 1H), 1.93-1.85 (m, 4H), 1.85- |
| | | | | 1.80 (m, 1H), 1.74-1.62 (m, 4H), 1.49 (s, 2H), 1.38 (d, J = |
| | | | | 6.4 Hz, 4H), 1.12 (d, J = 6.4 Hz, 2H), 0.91 (s, 8H) |
| I-181 | LL | HV | 943.5 | 12.34 (s, 1 H), 8.57-8.52 (m, 2H), 8.17-8.06 (m, 2H), 7.46 |
| | | | | (t, J = 8.4 Hz, 1H), 7.34-7.26 (m, 2H), 7.19 (d, J = 7.6 Hz, |
| | | | | 1H), 6.97 (t, J = 5.6 Hz, 2H), 5.67 (d, J = 9.2 Hz, 1H), 5.12 |
| | | | | (d, J = 3.6 Hz, 1H), 4.4 (d, J = 8.4 Hz, 2H), 4.38 (d, J = 2 Hz, |
| | | | | 1H), 4.37 (d, J = 5.6 Hz, 2H), 4.30 (s, 1H), 4.27-4.24 (m, |
| | | | | 2H), 3.88 (d, J = 8 Hz, 2H), 3.82 (d, J = 8.4 Hz, 1H), 3.78 (d, |
| | | | | J = 8.8 Hz, 1H), 3.73 (d, J = 8 Hz, 1H), 3.19-3.07 (m, 3H), |
| | | | | 3.06-2.79 (m, 8H), 2.77-2.69 (m, 2H), 2.29 (br d, J = 10.01 |
| | | | | Hz, 2H), 2.08-2.03 (m, 2 H), 2.00 (br d, J = 7.13 Hz, 4H), |
| | | | | 1.97-1.82 (m, 6H), 1.78-1.50 (m, 6H), 1.42 (d, J = 8 Hz, |
| | | | | 3H), 0.91 (s, 9H) |
| I-182 | LM | HV | 943.5 | 12.34 (s, 1H), 8.57-8.52 (m, 2H), 8.17-8.06 (m, 2H), 7.46 |
| | | | | (t, J = 8.4 Hz, 1H), 7.34-7.26 (m, 2H), 7.19 (d, J = 7.6 Hz, |
| | | | | 1H), 6.97 (t, J = 5.6 Hz, 2H), 5.67 (d, J = 9.2 Hz, 1H), 5.12 |
| | | | | (d, J = 3.6 Hz, 1H), 4.4 (d, J = 8.4 Hz, 2H), 4.38 (d, J = 2 Hz, |
| | | | | 1H), 4.37 (d, J = 5.6 Hz, 2H), 4.30 (s, 1H), 4.27-4.24 (m, |
| | | | | 2H), 3.88 (d, J = 8 Hz, 2H), 3.82 (d, J = 8.4 Hz, 1H), 3.78 |
| | | | | (d, J = 8.8 Hz, 1H), 3.73 (d, J = 8 Hz, 1H), 3.19-3.07 (m, 3 |
| | | | | H), 3.06-2.79 (m, 8 H), 2.77-2.69 (m, 2H), 2.29 (br d, J = |
| | | | | 10.01 Hz, 2H), 2.08-2.03 (m, 2H), 2.00 (br d, J = 7.13 Hz, |
| | | | | 4H), 1.97-1.82 (m, 6H), 1.78-1.50 (m, 6H), 1.42 (d, J = 8 |
| | | | | Hz, 3H), 0.91 (s, 9H) |
| I-183 | MO | MU | 955.6 | 14.50-14.14 (m, 1H), 12.29 (s, 1H), 8.52 (s, 1H), 8.42-8.35 |
| | | | | (m, 1H), 8.14 (s, 1H), 8.10 (d, J = 7.2 Hz, 1H), 7.34-7.25 |
| | | | | (m, 2H), 7.02 (d, J = 1.2 Hz, 1H), 6.98-6.91 (m, 3H), 5.69- |
| | | | | 5.60 (m, 1H), 5.13 (s, 1H), 4.42 (t, J = 8.0 Hz, 1H), 4.31 (t, |
| | | | | J = 8.8 Hz, 3H), 4.27-4.20 (m, 1H), 4.15-4.09 (m, 2H), |
| | | | | 3.90-3.84 (m, 1H), 3.81 (s, 3H), 3.76 (d, J = 9.2 Hz, 1H), |
| | | | | 3.70 (d, J = 8.4 Hz, 1H), 3.62 (s, 2H), 3.11-3.03 (m, 5H), |
| | | | | 2.87-2.80 (m, 3H), 2.73-2.69 (m, 1H), 2.54 (s, 1H), 2.52 |
| | | | | (d, J = 1.6 Hz, 2H), 2.26-2.20 (m, 2H), 2.04-1.98 (m, 2H), |
| | | | | 1.96-1.83 (m, 6H), 1.81-1.69 (m, 4H), 1.62-1.51 (m, 2H), |
| | | | | 1.39 (d, J = 6.4 Hz, 3H), 1.27-1.21 (m, 1H), 0.90 (s, 9H) |
| I-184 | MZ | HI | 924.6 | 0.92 (s, 9 H) 1.00 (br s, 4 H) 1.08-1.16 (m, 2 H) 1.39 (d, |
| | | | | J = 6.4 Hz, 3 H) 1.58 (br d, J = 10.4 Hz, 5 H) 1.71-1.80 (m, 2 |
| | | | | H) 1.84-1.93 (m, 5 H) 1.96-2.04 (m, 2 H) 2.17-2.23 (m, |
| | | | | 2 H) 2.32-2.35 (m, 1 H) 2.66-2.69 (m, 1 H) 2.70-2.84 (m, |
| | | | | 4 H) 2.98-3.05 (m, 1 H) 3.07-3.14 (m, 1 H) 3.63 (br s, 2 |
| | | | | H) 3.68-3.76 (m, 2 H) 3.79-3.87 (m, 2 H) 4.13 (s, 1 H) |
| | | | | 4.15-4.22 (m, 1 H) 4.28-4.41 (m, 5 H) 5.10-5.13 (m, 1 H) |
| | | | | 5.63 (br d, J = 9, 2 Hz, 1 H) 6.93-6.98 (m, 2 H) 7.28-7.34 |
| | | | | (m, 3 H) 7.35-7.39 (m, 2 H) 8.12 (d, J = 8.0 Hz, 1 H) 8.48- |
| | | | | 8.57 (m, 2 H) 12.24-12.30 (m, 1 H) 14.36 (s, 1 H) |
| I-185 | MZ | HI | 924.6 | 0.92 (s, 9 H) 1.00-1.09 (m, 4 H) 1.10-1.17 (m, 2 H) 1.35 |
| | | | | (br d, J = 6.4 Hz, 3 H) 1.37-1.48 (m, 5 H) 1.56-1.61 (m, 2 |
| | | | | H) 1.67 (br d, J = 9.6 Hz, 2H) 1.83-1.91 (m, 5 H) 1.97-2.05 |
| | | | | (m, 2 H) 2.21 (br dd, J = 8.4, 6.50 Hz, 2 H) 2.32-2.35 (m, 1 |
| | | | | H) 2.66-2.70 (m, 3 H) 2.74-2.78 (m, 2 H) 2.82-2.91 (m, |
| | | | | 2H) 2.97-3.01 (m, 1 H) 3.11-3.15 (m, 1 H) 3.63 (br s, 2 |
| | | | | H) 3.78-3.87 (m, 2 H) 4.13 (s, 1 H) 4.19 (br dd, J = 15.6, 5.2 |
| | | | | Hz, 1 H) 4.30-4.41 (m, 5 H) 5.11 (d, J = 3.2 Hz, 1 H) 5.63 |
| | | | | (br d, J = 9, 2 Hz, 1 H) 6.92-6.97 (m, 2 H) 7.26-7.33 (m, 3 |
| | | | | H) 7.35-7.39 (m, 2 H) 8.12 (br d, J = 8.0 Hz, 1 H) 8.51-8.57 |
| | | | | (m, 2 H) 12.23-12.28 (m, 1 H) 14.41 (br s, 1 H) |
| I-186 | LT | HI | 925.5 | 12.28 (br s, 1H), 8.57-8.46 (m, 2H), 8.21-8.07 (m, 2H), |
| | | | | 7.41-7.33 (m, 2H), 7.33-7.24 (m, 3H), 7.01-6.89 (m, 2H), |
| | | | | 5.63 (d, J = 9.4 Hz, 1H), 5.26-4.93 (m, 1H), 4.43-4.35 (m, |
| | | | | 2H), 4.34-4.27 (m, 3H), 4.18 (br dd, J = 5.5, 16.0 Hz, 1H), |
| | | | | 4.12 (s, 1H), 3.88-3.84 (m, 1H), 3.80 (br d, J = 8.0 Hz, 2H), |
| | | | | 3.77-3.72 (m, 2H), 3.70 (br d, J = 8.6 Hz, 2H), 3.62 (br s, |
| | | | | 1H), 3.15-3.06 (m, 2H), 3.04-2.98 (m, 1H), 2.88-2.79 (m, |
| | | | | 1H), 2.75-2.64 (m, 2H), 2.54 (br s, 1H), 2.52 (br d, J = 1.9 |
| | | | | Hz, 2H), 2.36-2.31 (m, 1H), 2.31-2.14 (m, 6H), 2.04-1.96 |
| | | | | (m, 2H), 1.94-1.80 (m, 6H), 1.39 (d, J = 6.4 Hz, 3H), 1.33- |
| | | | | 1.21 (m, 4H), 0.91 (s, 8H) |
| I-187 | LV | HI | 925.8 | 12.31 (br s, 1H), 8.61-8.47 (m, 2H), 8.14-8.08 (m, 1H), |
| | | | | 7.40-7.34 (m, 2H), 7.33-7.25 (m, 3H), 6.99-6.92 (m, 2H), |
| | | | | 5.66 (br d, J = 9.3 Hz, 1H), 5.11 (d, J = 3.3 Hz, 1H), 4.51- |
| | | | | 4.42 (m, 1H), 4.39 (br d, J = 7.8 Hz, 1H), 4.36-4.27 (m, 3H), |
| | | | | 4.18 (br dd, J = 5.6, 15.9 Hz, 1H), 4.12 (s, 1H), 3.90-3.85 |
| | | | | (m, 1H), 3.81 (br d, J = 7.9 Hz, 1H), 3.79-3.75 (m, 1H), 3.74- |
| | | | | 3.69 (m, 1H), 3.62 (br s, 3H), 3.22-3.13 (m, 3H), 3.09- |
| | | | | 2.99 (m, 2H), 2.98-2.84 (m, 3H), 2.74 (br d, J = 8.9 Hz, 4H), |
| | | | | 2.54 (br s, 1H), 2.52 (d, J = 1.9 Hz, 2H), 2.26 (br s, 2H), 2.12- |
| | | | | 2.03 (m, 2H), 2.03-1.93 (m, 3H), 1.91-1.82 (m, 2H), 1.71 |
| | | | | (br d, J = 4.0 Hz, 3H), 1.59-1.44 (m, 2H), 1.38 (br d, J = 5.6 |
| | | | | Hz, 3H), 0.91 (s, 9H) |
| I-188^{g} | MW | HI | 911.6 | 12.29 (s, 1H), 8.55-8.50 (m, 2H), 8.20 (s, 2H), 8.11 (d, J = |
| | | | | 8.0 Hz, 1H), 7.39-7.34 (m, 2H), 7.31 (s, 1H), 7.30-7.25 |
| | | | | (m, 2H), 6.97-6.92 (m, 2H), 5.65-5.59 (m, 1H), 4.41-4.35 |
| | | | | (m, 2H), 4.32 (t, J = 9.2 Hz, 3H), 4.23 (d, J = 6.4 Hz, 1H), |
| | | | | 4.20 (d, J = 5.2 Hz, 1H), 4.16 (d, J = 5.6 Hz, 1H), 4.11 (s, |
| | | | | 1H), 3.74 (s, 2H), 3.37-3.29 (m, 3H), 3.06 (d, J = 8.4 Hz, |
| | | | | 2H), 3.02-2.94 (m, 2H), 2.94-2.85 (m, 2H), 2.77-2.66 (m, |
| | | | | 4H), 2.61 (s, 1H), 2.54 (s, 1H), 2.45-2.39 (m, 1H), 2.24- |
| | | | | 2.15 (m, 2H), 2.09-1.98 (m, 3H), 1.94-1.85 (m, 3H), 1.80- |
| | | | | 1.68 (m, 4H), 1.38-1.33 (m, 4H), 0.91 (s, 9H) |
| I-189 | MM | HI | 928 | 0.94 (s, 8 H) 0.95-0.96 (m, 1 H) 1.36-1.47 (m, 6 H) 1.61- |
| | | | | 1.71 (m, 5 H) 1.81-1.92 (m, 5 H) 2.01-2.10 (m, 3 H) 2.20- |
| | | | | 2.27 (m, 3 H) 2.33-2.34 (m, 1 H) 2.65-2.71 (m, 4 H) 2.83- |
| | | | | 2.97 (m, 7 H) 3.04-3.12 (m, 5 H) 3.62-3.71 (m, 5 H) 3.88- |
| | | | | 3.99 (m, 3H) 4.12 (s, 1 H) 4.16-4.22 (m, 1H) 4.32-4.42 |
| | | | | (m, 5 H) 5.73 (br d, J = 9.2 Hz, 1 H) 6.92-6.98 (m, 2 H) 7.26- |
| | | | | 7.40 (m, 5 H) 8.11 (dd, J = 8.4, 1.6 Hz, 1 H) 8.18 (s, 1 H) |
| | | | | 8.51-8.56 (m, 2 H) |
| I-190 | LW | HI | 940 | 12.28 (s, 1H), 8.68-8.36 (m, 2H), 8.20 (s, 1H), 8.10 (dd, J = |
| | | | | 1.2, 8.4 Hz, 1H), 7.40-7.34 (m, 2H), 7.33-7.24 (m, 3H), |
| | | | | 6.98-6.91 (m, 2H), 5.60 (d, J = 9.6 Hz, 1H), 4.46-4.25 (m, |
| | | | | 6H), 4.21-4.14 (m, 1H), 4.12 (s, 1H), 3.89-3.81 (m, 3H), |
| | | | | 3.73 (d, J = 8.4 Hz, 2H), 3.14-3.01 (m, 3H), 3.00-2.92 (m, |
| | | | | 2H), 2.90-2.81 (m, 3H), 2.73-2.59 (m, 2H), 2.39-2.24 (m, |
| | | | | 6H), 2.20 (d, J = 12.0 Hz, 2H), 2.08-1.94 (m, 2H), 1.93- |
| | | | | 1.83 (m, 4H), 1.81-1.65 (m, 4H), 1.58-1.40 (m, 4H), 1.38 |
| | | | | (d, J = 6.4 Hz, 3H), 0.91 (s, 8H) |
| I-191 | LG | HI | 842.8 | 14.56-14.05 (m, 1H), 12.44-12.20 (m, 1H), 8.58 (d, J = 2.8 |
| | | | | Hz, 2H), 8.14 (s, 1H), 8.10 (dd, J = 1.6, 8.4 Hz, 1H), 7.41- |
| | | | | 7.33 (m, 2H), 7.32-7.26 (m, 3H), 7.03-6.88 (m, 2H), 5.72 |
| | | | | (d, J = 9.2 Hz, 1H), 5.11 (d, J = 3.2 Hz, 1H), 4.42-4.28 (m, |
| | | | | 5H), 4.22-4.14 (m, 1H), 4.12 (s, 1H), 3.92-3.86 (m, 1H), |
| | | | | 3.85-3.78 (m, 2H), 3.77-3.72 (m, 1H), 3.62 (s, 2H), 3.17- |
| | | | | 3.03 (m, 5H), 2.97-2.83 (m, 2H), 2.82-2.68 (m, 2H), 2.36 |
| | | | | (s, 2H), 2.15 (s, 2H), 2.06-1.93 (m, 3H), 1.92-1.80 (m, 2H), |
| | | | | 1.69-1.51 (m, 2H), 1.41 (d, J = 6.4 Hz, 3H), 0.91 (s, 8H) |
| I-193 | ND | HI | 927.6 | 0.93 (s, 9 H) 1.38 (br d, J = 6.25 Hz, 5 H) 1.51 (br d, J = 0.75 |
| | | | | Hz, 8 H) 1.80-1.93 (m, 4 H) 1.97-2.05 (m, 2 H) 2.17 (br d, |
| | | | | J = 7.38 Hz, 3 H) 2.26-2.34 (m, 6 H) 2.65-2.73 (m, 2 H) |
| | | | | 2.82-2.88 (m, 1 H) 2.98-3.10 (m, 5 H) 3.61-3.69 (m, 4 H) |
| | | | | 4.12 (s, 1 H) 4.16-4.22 (m, 1 H) 4.31-4.43 (m, 6 H) 5.82 |
| | | | | (br d, J = 9.01 Hz, 1 H) 6.95 (d, J = 8.00 Hz, 2 H) 7.25-7.33 |
| | | | | (m, 3 H) 7.36 (s, 2 H) 8.11 (br d, J = 7.88 Hz, 1 H) 8.22 (s, 2 |
| | | | | H) 8.51-8.56 (m, 2 H) |
| I-194 | NE | HI | 927.6 | 0.93 (s, 9 H) 1.38 (br d, J = 6.25 Hz, 5 H) 1.51 (br d, J = 0.75 |
| | | | | Hz, 8 H) 1.80-1.93 (m, 4 H) 1.97-2.05 (m, 2 H) 2.17 (br d, |
| | | | | J = 7.38 Hz, 3 H) 2.26-2.34 (m, 6 H) 2.65-2.73 (m, 2 H) |
| | | | | 2.82-2.88 (m, 1 H) 2.98-3.10 (m, 5 H) 3.61-3.69 (m, 4 H) |
| | | | | 4.12 (s, 1 H) 4.16-4.22 (m, 1 H) 4.31-4.43 (m, 6 H) 5.82 |
| | | | | (br d, J = 9.01 Hz, 1 H) 6.95 (d, J = 8.00 Hz, 2 H) 7.25-7.33 |
| | | | | (m, 3 H) 7.36 (s, 2 H) 8.11 (br d, J = 7.88 Hz, 1 H) 8.22 (s, 2 |
| | | | | H) 8.51-8.56 (m, 2 H) |
| I-198 | LP | HI | 844.5 | 14.41-14.27 (m, 1H), 12.30 (s, 1H), 8.55-8.51 (m, 2H), |
| | | | | 8.13-8.10 (m, 1H), 7.40-7.36 (m, 2H), 7.32-7.27 (m, 3H), |
| | | | | 6.98-6.93 (m, 2H), 5.76 (d, J = 8.8 Hz, 1H), 5.16-5.10 (m, |
| | | | | 1H), 4.49-4.31 (m, 6H), 4.23-4.16 (m, 1H), 4.13 (s, 1H), |
| | | | | 3.99-3.90 (m, 2H), 3.69-3.63 (m, 2H), 3.14-3.06 (m, 2H), |
| | | | | 2.94-2.82 (m, 2H), 2.73-2.63 (m, 4H), 2.55 (s, 1H), 2.53 |
| | | | | (d, J = 2.0 Hz, 2H), 2.42-2.42 (m, 1H), 2.06-1.98 (m, 2H), |
| | | | | 1.93-1.84 (m, 3H), 1.83-1.77 (m, 1H), 1.66 (t, J = 12.0 Hz, |
| | | | | 3H), 1.61-1.48 (m, 2H), 1.40 (d, J = 6.4 Hz, 3H), 1.20-1.16 |
| | | | | (m, 2H), 0.94 (s, 9H) |
| I-199 | LZ | HI | 844.5 | 14.36 (s, 1H), 12.38-12.07 (m, 1H), 8.59-8.46 (m, 2H), |
| | | | | 8.11 (d, J = 8.4 Hz, 1H), 7.39-7.36 (m, 1H), 7.31 (d, J = 8.0 |
| | | | | Hz, 2H), 7.28-7.25 (m, 1H), 6.98-6.91 (m, 2H), 5.83 (d, J = |
| | | | | 8.8 Hz, 1H), 5.10 (d, J = 3.6 Hz, 1H), 4.49-4.24 (m, 5H), |
| | | | | 4.23-4.08 (m, 2H), 3.72-3.59 (m, 2H), 3.30-3.25 (m, 5H), |
| | | | | 3.19-3.06 (m, 1H), 3.04-2.93 (m, 1H), 2.91-2.76 (m, 1H), |
| | | | | 2.75-2.60 (m, 2H), 2.37-2.19 (m, 5H), 2.09-1.76 (m, 8H), |
| | | | | 1.48-1.20 (m, 6H), 0.96-0.89 (m, 9H) |
| I-200 | LZ | HI | 844.7 | 14.45.14.36 (m, 1H), 12.50-11.89 (m, 1H), 9.37 (d, J = 2.4 |
| | | | | Hz, 1H), 8.79 (dd, J = 1.2, 4.4 Hz, 1H), 8.63 (s, 1H), 8.58 (s, |
| | | | | 2H), 8.19 (s, 1H), 8.16-8.06 (m, 1H), 7.40-7.35 (m, 2H), |
| | | | | 7.31 (d, J = 8.0 Hz, 2H), 7.28-7.25 (m, 1H), 6.98-6.91 (m, |
| | | | | 2H), 5.82 (d, J = 8.8 Hz, 1H), 5.10 (d, J = 3.6 Hz, 1H), 4.45- |
| | | | | 4.30 (m, 5H), 4.25-4.07 (m, 2H), 3.74-3.58 (m, 2H), 3.19- |
| | | | | 3.09 (m, 1H), 3.05-2.96 (m, 1H), 2.93-2.78 (m, 1H), 2.70- |
| | | | | 2.62 (m, 2H), 2.37-2.23 (m, 5H), 2.21-2.11 (m, 2H), 2.05- |
| | | | | 1.96 (m, 1H), 1.87 (ddd, J = 4.4, 8.4, 12.9 Hz, 1H), 1.82- |
| | | | | 1.66 (m, 3H), 1.58-1.40 (m, 6H), 1.35 (d, J = 6.4 Hz, 3H), |
| | | | | 0.93 (s, 9H) |
| I-203 | NI | HI | 927.9 | 8.58-8.43 (m, 2H), 8.16-8.04 (m, 1H), 7.40-7.34 (m, 2H), |
| | | | | 7.33-7.23 (m, 3H), 6.94 (d, J = 7.6 Hz, 2H), 5.82 (d, J = 9.2 |
| | | | | Hz, 1H), 5.19-5.05 (m, 1H), 4.37 (dd, J = 6.8, 15.6 Hz, 4H), |
| | | | | 4.27 (q, J = 6.0 Hz, 1H), 4.18 (dd, J = 5.6, 16.0 Hz, 1H), 4.12 |
| | | | | (s, 1H), 3.71-3.61 (m, 2H), 3.17 (d, J = 8.0 Hz, 1H), 3.12- |
| | | | | 3.06 (m, 1H), 3.03-2.94 (m, 1H), 2.90-2.68 (m, 4H), 2.59- |
| | | | | 2.53 (m, 1H), 2.52 (d, J = 2.0 Hz, 2H), 2.25 (s, 5H), 2.15- |
| | | | | 2.06 (m, 4H), 2.04-1.97 (m, 2H), 1.87 (ddd, J = 4.8, 8.4, |
| | | | | 12.8 Hz, 2H), 1.77 (d, J = 11.2 Hz, 2H), 1.69-1.48 (m, 4H), |
| | | | | 1.38 (d, J = 6.4 Hz, 3H), 1.34-1.20 (m, 5H), 1.02 (d, J = 9.2 |
| | | | | Hz, 2H), 0.93 (s, 9H) |
| I-204 | NI | HI | 928.7 | 8.60-8.47 (m, 2H), 8.29-8.20 (m, 2H), 8.11 (d, J = 8.0 Hz, |
| | | | | 1H), 7.43-7.34 (m, 2H), 7.33-7.25 (m, 3H), 6.99-6.91 (m, |
| | | | | 2H), 5.84 (d, J = 8.8 Hz, 1H), 4.40-4.34 (m, 4H), 4.18 (dd, |
| | | | | J = 5.2, 16.0 Hz, 2H), 4.12 (s, 1H), 3.72-3.61 (m, 3H), 3.29 |
| | | | | (d, J = 10.4 Hz, 5H), 3.17-2.86 (m, 6H), 2.68 (s, 2H), 2.54 |
| | | | | (d, J = 1.6 Hz, 1H), 2.26 (s, 4H), 2.13-1.98 (m, 5H), 1.89- |
| | | | | 1.80 (m, 2H), 1.73 (d, J = 10.0 Hz, 2H), 1.62-1.40 (m, 5H), |
| | | | | 1.35 (d, J = 5.6 Hz, 3H), 1.21-1.07 (m, 2H), 0.93 (s, 9H) |
| I-205 | ME | HI | 937.9 | 12.35 (br s, 1H), 8.56-8.51 (m, 2H), 8.17 (s, 1H), 8.10 (dd, |
| | | | | J = 1.4, 8.4 Hz, 1H), 7.36 (br d, J = 6.0 Hz, 1H), 7.34 (d, J = |
| | | | | 7.2 Hz, 2H), 7.30 (d, J = 7.6 Hz, 1H), 6.99-6.94 (m, 2H), |
| | | | | 6.21-6.15 (m, 2H), 4.44-4.40 (m, 1H), 4.38 (br d, J = 7.6 |
| | | | | Hz, 2H), 4.33 (br d, J = 9.6 Hz, 2H), 4.19 (br d, J = 5.6 Hz, |
| | | | | 1H), 4.16-4.13 (m, 1H), 4.12 (s, 1H), 3.75 (br d, J = 14.4 |
| | | | | Hz, 2H), 3.63 (br s, 2H), 3.51 (br s, 2H), 3.37 (br s, 2H), 3.34- |
| | | | | 3.26 (m, 3H), 3.17-3.10 (m, 3H), 2.92 (br d, J = 10.0 Hz, |
| | | | | 2H), 2.83 (br s, 2H), 2.73-2.67 (m, 1H), 2.56 (br s, 1H), 2.54 |
| | | | | (s, 1H), 2.52 (br d, J = 2.0 Hz, 1H), 2.10 (br d, J = 6.8 Hz, |
| | | | | 3H), 2.06-1.99 (m, 4H), 1.92-1.80 (m, 3H), 1.50 (br d, J = |
| | | | | 5.2 Hz, 2H), 1.44 (br d, J = 6.4 Hz, 3H), 1.40 (br d, J = 2.4 |
| | | | | Hz, 1H), 0.91 (s, 9H) |
| I-207 | LR | HI | 937.4 | 12.29 (s, 1H), 8.57-8.48 (m, 2H), 8.23 (s, 1H), 8.12 (d, J = |
| | | | | 8.4 Hz, 1H), 7.42-7.34 (m, 2H), 7.33-7.24 (m, 3H), 7.00- |
| | | | | 6.90 (m, 2H), 5.62 (d, J = 9.2 Hz, 1H), 4.44-4.27 (m, 5H), |
| | | | | 4.25-4.14 (m, 2H), 4.13-4.08 (m, 2H), 3.37-3.23 (m, 4H), |
| | | | | 3.22-3.08 (m, 4H), 2.93-2.81 (m, 3H), 2.68-2.57 (m, 3H), |
| | | | | 2.26 (d, J = 6.4 Hz, 2H), 2.23-2.14 (m, 2H), 2.14-2.07 (m, |
| | | | | 1H), 2.06-1.95 (m, 2H), 1.93-1.81 (m, 4H), 1.75-1.64 (m, |
| | | | | 2H), 1.63-1.52 (m, 2H), 1.28 (d, J = 6.4 Hz, 3H), 1.17-1.00 |
| | | | | (m, 2H), 0.91 (s, 8H) |
| I-208 | MS | HI | 926.1 | 12.37-12.24 (m, 1H), 12.33-12.23 (m, 1H), 8.54 (s, 1H), |
| | | | | 8.24 (s, 2H), 8.13 (d, J = 7.2 Hz, 1H), 7.40-7.35 (m, 2H), |
| | | | | 7.33-7.24 (m, 3H), 6.97-6.95 (m, 1H), 6.95-6.95 (m, 1H), |
| | | | | 6.96-6.94 (m, 1H), 6.95 (d, J = 7.6 Hz, 1H), 5.82 (d, J = 8.8 |
| | | | | Hz, 1H), 4.42-4.33 (m, 5H), 4.23-4.19 (m, 1H), 4.22-4.19 |
| | | | | (m, 1H), 4.23-4.19 (m, 1H), 4.17-4.15 (m, 1H), 4.18-4.15 |
| | | | | (m, 1H), 4.25-4.15 (m, 1H), 4.22-4.15 (m, 1H), 4.13-4.10 |
| | | | | (m, 2H), 3.99 (s, 2H), 3.71-3.61 (m, 3H), 3.44-3.38 (m, |
| | | | | 2H), 3.30 (s, 2H), 3.16-3.10 (m, 1H), 2.91-2.79 (m, 5H), |
| | | | | 2.70-2.57 (m, 4H), 2.32-2.25 (m, 2H), 2.14 (t, J = 10.4 Hz, |
| | | | | 2H), 2.05-1.96 (m, 2H), 1.94-1.83 (m, 2H), 1.72-1.57 (m, |
| | | | | 4H), 1.32-1.32 (m, 1H), 1.31-1.27 (m, 3H), 1.26-1.11 (m, |
| | | | | 3H), 0.93 (s, 8H) |
| I-210 | LL | MP | 951.6 | 12.27 (br s, 1H), 8.74 (s, 1H), 8.52 (s, 1H), 8.26 (s, 1H), 8.11 |
| | | | | (dd, J = 1.4, 8.6 Hz, 1H), 7.32-7.24 (m, 3H), 7.24-7.17 (m, |
| | | | | 2H), 7.00-6.89 (m, 2H), 5.68 (br d, J = 9.3 Hz, 1H), 4.37- |
| | | | | 4.34 (m, 1H), 4.34-4.29 (m, 3H), 4.10-4.07 (m, 1H), 3.86 |
| | | | | (br d, J = 8.0 Hz, 1H), 3.81 (br d, J = 8.0 Hz, 1H), 3.75 (br d, |
| | | | | J = 8.5 Hz, 1H), 3.70 (br d, J = 9.0 Hz, 2H), 3.61 (br s, 3H), |
| | | | | 3.14-3.01 (m, 3H), 2.93-2.82 (m, 3H), 2.77 (br d, J = 10.5 |
| | | | | Hz, 2H), 2.69 (br d, J = 3.6 Hz, 1H), 2.54 (br s, 1H), 2.19 (br |
| | | | | d, J = 5.1 Hz, 3H), 2.17-2.09 (m, 2H), 2.04-1.94 (m, 2H), |
| | | | | 1.93-1.83 (m, 4H), 1.82-1.75 (m, 1H), 1.70-1.64 (m, 2H), |
| | | | | 1.64-1.59 (m, 1H), 1.52-1.41 (m, 2H), 1.41-1.34 (m, 4H), |
| | | | | 1.33 (br s, 1H), 1.21-1.15 (m, 3H), 1.13-1.08 (m, 1H), 0.91 |
| | | | | (s, 8H) |
| I-212 | PQ | LL | 939.7 | 12.37 (s, 1H), 8.54 (s, 1H), 8.37 (d, J = 7.6 Hz, 1H), 8.14 (s, |
| | | | | 1H), 8.10 (d, J = 7.6 Hz, 1H), 7.42 (d, J = 8 Hz, 2H), 7.28 (d, |
| | | | | J = 8 Hz, 3H), 6.98-6.94 (m, 2H), 5.60 (d, J = 8.8 Hz, 1H), |
| | | | | 5.10 (d, J = 3.2 Hz, 1H), 4.90-4.83 (m, 1H), 4.40-4.36 (t, |
| | | | | J = 16.0 Hz, 2H), 4.29 (m, 2H), 4.13 (s, 1H), 3.88-3.81 (m, |
| | | | | 2H), 3.74 (d, J = 8.4 Hz, 2H), 3.57 (s, 2H), 3.14 (s, 2H), 3.01- |
| | | | | 2.95 (m, 3H), 2.88-2.84 (m, 2H), 2.76-2.74 (m, 1H), 2.67 |
| | | | | (d, J = 2 Hz, 1H), 2.55 (s, 2H), 2.33-2.29 (m, 3H), 2.05- |
| | | | | 1.92 (m, 8H), 1.82-1.65 (m, 4H), 1.63-1.49 (m, 3H), 1.43 |
| | | | | (d, J = 6.4 Hz, 3H), 1.33 (br d, J = 8.0 Hz, 3H), 0.91 (s, 9H) |
| I-215 | NL | HI | 928.8 | 8.53 (t, J = 5.6 Hz, 1H), 8.13 (s, 1H), 8.12-8.12 (m, 1H), |
| | | | | 8.12-8.12 (m, 1H), 8.15-8.11 (m, 1H), 8.19-8.09 (m, 1H), |
| | | | | 7.39-7.35 (m, 2H), 7.33-7.26 (m, 4H), 7.24-7.19 (m, 1H), |
| | | | | 7.14 (s, 1H), 7.02 (s, 1H), 6.96-6.86 (m, 2H), 5.89-5.66 |
| | | | | (m, 1H), 5.15-5.08 (m, 1H), 4.43-4.27 (m, 5H), 4.22-4.11 |
| | | | | (m, 3H), 3.90 (d, J = 8.4 Hz, 1H), 3.87-3.80 (m, 2H), 3.78- |
| | | | | 3.74 (m, 1H), 3.63 (s, 2H), 3.53 (dd, J = 9.2, 11.1 Hz, 2H), |
| | | | | 3.46-3.37 (m, 3H), 3.24-3.14 (m, 4H), 3.12-2.73 (m, 7H), |
| | | | | 2.28-2.11 (m, 4H), 2.08-1.95 (m, 6H), 1.93-1.81 (m, 4H), |
| | | | | 1.27-1.20 (m, 1H), 1.20-1.12 (m, 1H), 0.92 (s, 9H) |
| I-216 | NU | HI | 930.4 | 8.53 (br t, J = 5.9 Hz, 1H), 8.24 (s, 2H), 7.91 (br d, J = 7.3 |
| | | | | Hz, 1H), 7.39-7.33 (m, 3H), 7.32-7.29 (m, 2H), 7.23-7.19 |
| | | | | (m, 2H), 6.87-6.83 (m, 2H), 5.84 (br d, J = 9.0 Hz, 1H), 4.42- |
| | | | | 4.38 (m, 2H), 4.35 (br d, J = 7.6 Hz, 3H), 4.18 (br d, J = |
| | | | | 10.4 Hz, 1H), 4.12 (s, 1H), 4.04 (br s, 1H), 3.67 (br s, 1H), |
| | | | | 3.50 (br d, J = 5.1 Hz, 1H), 3.30 (br d, J = 5.4 Hz, 1H), 3.17 |
| | | | | (br d, J = 6.1 Hz, 3H), 3.12 (br d, J = 10.9 Hz, 2H), 3.07 (br |
| | | | | d, J = 9.4 Hz, 2H), 2.85 (br t, J = 10.9 Hz, 2H), 2.27 (br s, |
| | | | | 5H), 2.23-2.17 (m, 2H), 2.11 (br d, J = 6.5 Hz, 3H), 2.05- |
| | | | | 1.96 (m, 2H), 1.92-1.84 (m, 3H), 1.79 (br s, 1H), 1.74 (br s, |
| | | | | 2H), 1.72 (br s, 2H), 1.51 (br s, 3H), 1.41 (br t, J = 10.3 Hz, |
| | | | | 2H), 1.24-1.07 (m, 3H), 0.93 (s, 9H) |
| I-217 | NV | HI | 930.5 | 8.53 (br t, J = 6.0 Hz, 1H), 8.22 (s, 2H), 7.91 (br d, J = 7.1 |
| | | | | Hz, 1H), 7.39-7.35 (m, 2H), 7.35-7.31 (m, 2H), 7.30 (s, |
| | | | | 1H), 7.22-7.17 (m, 2H), 6.87-6.82 (m, 2H), 5.84 (br d, J = |
| | | | | 9.0 Hz, 1H), 4.42-4.38 (m, 2H), 4.36 (br d, J = 8.1 Hz, 3H), |
| | | | | 4.18 (br d, J = 10.5 Hz, 1H), 4.12 (s, 1H), 4.01 (br s, 1H), |
| | | | | 3.67 (br s, 1H), 3.65 (br s, 1H), 3.47 (br s, 1H), 3.32 (br s, |
| | | | | 1H), 3.29 (br s, 1H), 3.16 (br d, J = 7.3 Hz, 2H), 3.03-2.89 |
| | | | | (m, 6H), 2.82 (br t, J = 10.6 Hz, 2H), 2.36 (br d, J = 10.9 Hz, |
| | | | | 3H), 2.27 (br s, 5H), 2.10 (br d, J = 6.6 Hz, 2H), 1.99 (br dd, |
| | | | | J = 4.8, 7.1 Hz, 2H), 1.89 (br s, 1H), 1.87 (br d, J = 3.8 Hz, |
| | | | | 2H), 1.71 (br d, J = 10.9 Hz, 2H), 1.58-1.49 (m, 1H), 1.37- |
| | | | | 1.28 (m, 2H), 1.28-1.22 (m, 2H), 1.13 (br d, J = 13.3 Hz, |
| | | | | 2H), 1.09 (br d, J = 6.8 Hz, 1H), 0.93 (s, 9H) |
| I-218 | NZ | HI | 932.7 | 14.76 (s, 1H), 8.54 (t, J = 6.2 Hz, 1H), 7.91 (d, J = 7.2 Hz, |
| | | | | 1H), 7.47-7.26 (m, 5H), 7.24-7.14 (m, 2H), 6.91-6.74 (m, |
| | | | | 2H), 5.94 (d, J = 8.8 Hz, 1H), 5.11 (d, J = 3.6 Hz, 1H), 4.47- |
| | | | | 4.30 (m, 4H), 4.23-4.15 (m, 1H), 4.12 (s, 1H), 4.03 (d, J = |
| | | | | 12.0 Hz, 1H), 3.89-3.73 (m, 3H), 3.73-3.60 (m, 2H), 3.51- |
| | | | | 3.35 (m, 3H), 3.22-3.10 (m, 3H), 3.06-2.99 (m, 2H), 2.91- |
| | | | | 2.82 (m, 4H), 2.36-2.25 (m, 5H), 2.24-2.16 (m, 2H), 2.15- |
| | | | | 2.05 (m, 3H), 2.03-1.96 (m, 2H), 1.95-1.87 (m, 3H), 1.79- |
| | | | | 1.71 (m, 2H), 1.68-1.60 (m, 2H), 1.39 (d, J = 10.0 Hz, 4H), |
| | | | | 1.16-1.12 (m, 1H), 0.99-0.89 (m, 9H) |
| I-219 | OD | HI | 932.7 | 8.55 (t, J = 6.0 Hz, 1H), 8.29 (d, J = 2.0 Hz, 1H), 7.91 (d, J = |
| | | | | 8.0 Hz, 1H), 7.39-7.29 (m, 5H), 7.23-7.17 (m, 2H), 6.89- |
| | | | | 6.79 (m, 2H), 5.93 (d, J = 8.8 Hz, 1H), 5.12 (dt, J = 2.4, 8.1 |
| | | | | Hz, 1H), 4.43-4.33 (m, 4H), 4.18 (dd, J = 5.6, 15.9 Hz, 1H), |
| | | | | 4.12 (s, 1H), 4.03 (d, J = 11.6 Hz, 1H), 3.90 (d, J = 12.8 Hz, |
| | | | | 1H), 3.80-3.64 (m, 5H), 3.20-3.12 (m, 5H), 3.08-2.98 (m, |
| | | | | 4H), 2.93-2.85 (m, 4H), 2.82-2.73 (m, 2H), 2.30 (d, J = 5.2 |
| | | | | Hz, 3H), 2.23-2.16 (m, 2H), 2.13-2.03 (m, 3H), 2.03-1.97 |
| | | | | (m, 1H), 1.92-1.92 (m, 1H), 1.93-1.85 (m, 2H), 1.75 (d, J = |
| | | | | 12.4 Hz, 2H), 1.68-1.62 (m, 2H), 1.51-1.28 (m, 5H), 0.97- |
| | | | | 0.91 (m, 9H) |
| I-224 | OB | HI | 940.7 | 8.55 (t, J = 6.0 Hz, 1H), 8.25 (s, 1H), 7.95-7.87 (m, 1H), |
| | | | | 7.38-7.35 (m, 2H), 7.33-7.27 (m, 2H), 7.24-7.17 (m, 2H), |
| | | | | 6.89-6.81 (m, 2H), 5.67-5.63 (m, 1H), 4.40-4.29 (m, 4H), |
| | | | | 4.20 (d, J = 5.6 Hz, 1H), 4.17-4.13 (m, 1H), 4.07-4.02 (m, |
| | | | | 1H), 3.87-3.78 (m, 3H), 3.71 (dd, J = 8.8, 18.4 Hz, 3H), |
| | | | | 3.62 (s, 4H), 3.17 (d, J = 9.6 Hz, 3H), 3.14 (s, 2H), 3.07- |
| | | | | 2.99 (m, 3H), 2.90 (d, J = 7.6 Hz, 3H), 2.85-2.78 (m, 2H), |
| | | | | 2.23-2.15 (m, 4H), 2.04-1.96 (m, 2H), 1.95-1.84 (m, 7H), |
| | | | | 1.71-1.63 (m, 2H), 1.60-1.51 (m, 3H), 0.91 (s, 8H) |
| I-227 | HZ | OG | 1009.4 | 12.44 (s, 1H), 8.71 (s, 1H), 8.40 (t, J = 5.9 Hz, 1H), 8.4 (s, |
| | | | | 2H), 8.22-8.16 (m, 1H), 7.35-7.26 (m, 2H), 7.02-6.99 (m, |
| | | | | 1H), 6.99-6.90 (m, 3H), 6.04 (q, J = 6.3 Hz, 1H), 5.6 (d, J = |
| | | | | 9.3 Hz, 1H), 5.17-4.98 (m, 2H), 4.44 (t, J = 8.1 Hz, 1H), |
| | | | | 4.32 (d, J = 9.5 Hz, 2H), 4.24 (dd, J = 6.5, 16.9 Hz, 1H), 4.17- |
| | | | | 4.09 (m, 2H), 4.00 (t, J = 6.2 Hz, 2H), 3.90-3.86 (m, 1H), |
| | | | | 3.83 (br d, J = 8.3 Hz, 1H), 3.78-3.75 (m, 1H), 3.74-3.70 |
| | | | | (m, 1H), 3.64 (s, 2H), 3.48 (t, J = 6.3 Hz, 3H), 3.24 (s, 3H), |
| | | | | 3.01-2.89 (m, 2H), 2.84 (d, J = 9.6 Hz, 2H), 2.62-2.55 (m, |
| | | | | 1H), 2.56 (s, 1H), 2.41-2.36 (m, 1H), 2.24 (t, J = 8.2 Hz, |
| | | | | 2H), 2.05-1.98 (m, 1H), 1.97-1.94 (m, 2H), 1.93-1.84 (m, |
| | | | | 3H), 1.80-1.67 (m, 4H), 1.65-1.56 (m, 2H), 1.52 (d, J = |
| | | | | 6.5 Hz, 3H), 0.90 (s, 9H) |
| I-228 | HT | HI | 920.6 | 14.46-14.16 (m, 1H), 12.49-12.40 (m, 1H), 8.71 (s, 1H), |
| | | | | 8.54 (t, J = 6.0 Hz, 1H), 8.34 (s, 2H), 8.21 (J = 1.2, 8.4 Hz, |
| | | | | 1H), 7.40-7.35 (m, 2H), 7.31 (d, J = 8.4 Hz, 2H), 7.28 (d, J = |
| | | | | 1.2 Hz, 1H), 6.99-6.94 (m, 2H), 6.04 (q, J = 6.4 Hz, 1H), |
| | | | | 5.67 (d, J = 9.6 Hz, 1H), 5.12 (d, J = 3.2 Hz, 1H), 5.05 (J = |
| | | | | 4.0, 12.8 Hz, 1H), 4.43-4.39 (m, 1H), 4.40 (s, 1H), 4.37 (d, |
| | | | | J = 2.0 Hz, 1H), 4.38-4.30 (m, 1H), 4.31 (s, 1H), 4.21 (d, J = |
| | | | | 5.6 Hz, 1H), 4.23-4.16 (m, 1H), 4.17 (d, J = 5.6 Hz, 1H), |
| | | | | 4.13 (s, 1H), 3.92-3.87 (m, 1H), 3.86-3.82 (m, 1H), 3.81- |
| | | | | 3.77 (m, 1H), 3.76-3.71 (m, 1H), 3.64 (s, 2H), 3.04-2.82 |
| | | | | (m, 5H), 2.78-2.65 (m, 1H), 2.45-2.39 (m, 1H), 2.34-2.26 |
| | | | | (m, 2H), 2.06-1.95 (m, 4H), 1.93-1.83 (m, 2H), 1.76 (d, J = |
| | | | | 11.6 Hz, 2H), 1.68-1.59 (m, 2H), 1.55 (d, J = 6.8 Hz, 3H), |
| | | | | 0.93 (s, 9H) |
| I-245^{h} | PE | HI | 911.6 | 14.72 (s, 1H), 8.54 (t, J = 5.6 Hz, 1H), 8.34 (s, 2H), 7.94 (d, |
| | | | | J = 8.0 Hz, 1H), 7.43-7.35 (m, 3H), 7.34-7.28 (m, 3H), |
| | | | | 7.21 (t, J = 7.6 Hz, 1H), 6.91-6.81 (m, 2H), 5.85 (s, 1H), |
| | | | | 5.11 (d, J = 3.2 Hz, 1H), 4.77-4.67 (m, 2H), 4.44-4.32 (m, |
| | | | | 4H), 4.19 (d, J = 10.6 Hz, 2H), 4.14-4.09 (m, 1H), 4.07- |
| | | | | 4.00 (m, 1H), 3.87 (t, J = 13.6 Hz, 1H), 3.75-3.56 (m, 3H), |
| | | | | 3.29-3.19 (m, 2H), 3.17-3.01 (m, 2H), 3.00-2.94 (m, 2H), |
| | | | | 2.80-2.61 (m, 3H), 2.39 (s, 1H), 2.31-2.18 (m, 3H), 2.07- |
| | | | | 1.97 (m, 1H), 1.91-1.84 (m, 2H), 1.74-1.69 (m, 2H), 1.68- |
| | | | | 1.56 (m, 3H), 1.38 (d, J = 1.6 Hz, 2H), 1.25-1.22 (m, 1H), |
| | | | | 0.94 (s, 9H) |
| I-249 | OZ | JE | 468.3 | 0.92 (s, 9 H) 1.32 (br d, J = 7.00 Hz, 3 H) 1.52 (br d, J = 6.50 |
| | | | (M/2 + H)⁺ | Hz, 3 H) 1.70-1.83 (m, 3 H) 1.96-2.02 (m, 1 H) 2.56-2.69 |
| | | | | (m, 3 H) 2.84-3.00 (m, 3 H) 3.39 (br s, 3 H) 3.57 (br s, 4 H) |
| | | | | 3.63 (br d, J = 10.26 Hz, 3 H) 3.83-4.01 (m, 5 H) 4.10-4.13 |
| | | | | (m, 1 H) 4.11-4.12 (m, 1 H) 4.12 (s, 1 H) 4.26 (br s, 1 H) |
| | | | | 4.31 (br d, J = 9.26 Hz, 1 H) 4.38 (br t, J = 8.00 Hz, 1 H) 4.87 |
| | | | | (br t, J = 7.19 Hz, 1 H) 4.96 (br dd, J = 13.07, 4.32 Hz, 1 H) |
| | | | | 5.09 (br s, 1 H) 5.78 (br d, J = 9.01 Hz, 1 H) 5.97 (q, J = 6.13 |
| | | | | Hz, 1 H) 6.93-6.98 (m, 2 H) 7.25-7.31 (m, 3 H) 7.41 (d, |
| | | | | J = 8.13 Hz, 2 H) 8.17-8.26 (m, 3 H) 8.35 (br d, J = 7.75 Hz, |
| | | | | 1 H) 8.33-8.37 (m, 1 H) 8.69 (s, 1 H) 12.42 (br s, 1 H) |
| I-250 | OU | JE | 934.6 | 12.46 (s, 1H), 8.66 (s, 1H), 8.43-8.33 (m, 2H), 8.18-8.13 |
| | | | | (m, 1H), 8.04 (s, 1H), 7.42 (d, J = 8.0 Hz, 2H), 7.33-7.25 |
| | | | | (m, 3H), 7.02-6.93 (m, 2H), 5.88-5.78 (m, 1H), 5.67-5.57 |
| | | | | (m, 1H), 5.10 (s, 1H), 4.92-4.83 (m, 1H), 4.72-4.64 (m, |
| | | | | 1H), 4.40 (t, J = 8.0 Hz, 1H), 4.34-4.24 (m, 2H), 4.12 (s, |
| | | | | 1H), 3.91-3.83 (m, 2H), 3.82-3.73 (m, 2H), 3.58 (s, 2H), |
| | | | | 3.55-3.45 (m, 2H), 3.42-3.38 (m, 2H), 3.11-3.00 (m, 3H), |
| | | | | 2.94-2.87 (m, 1H), 2.72-2.65 (m, 1H), 2.55 (s, 3H), 2.35 ( |
| | | | | d, J = 7.6 Hz, 2H), 2.11 (s, 2H), 2.03-1.97 (m, 1H), 1.87 ( |
| | | | | d, J = 9.6 Hz, 2H), 1.79-1.72 (m, 2H), 1.56 (d, J = 6.4 Hz, |
| | | | | 3H), 1.33 (d, J = 6.8 Hz, 3H), 0.92 (s, 9H) |
| I-253 | NO | HI | 930.7 | 8.56 (br t, J = 5.8 Hz, 1H), 8.17 (s, 1H), 7.92 (br d, J = 7.8 |
| | | | | Hz, 1H), 7.41-7.36 (m, 2H), 7.34 (br d, J = 2.9 Hz, 1H), |
| | | | | 7.33-7.29 (m, 2H), 7.23-7.17 (m, 2H), 6.87-6.81 (m, 2H), |
| | | | | 5.67 (br d, J = 9.3 Hz, 1H), 4.44-4.38 (m, 2H), 4.36 (br d, J = |
| | | | | 9.0 Hz, 2H), 4.18 (br dd, J = 5.4, 16.3 Hz, 1H), 4.13 (s, |
| | | | | 1H), 4.03 (br d, J = 11.8 Hz, 1H), 3.73 (br s, 1H), 3.69 (br s, |
| | | | | 2H), 3.67-3.63 (m, 2H), 3.15 (br d, J = 9.1 Hz, 2H), 3.02 (br |
| | | | | d, J = 8.3 Hz, 2H), 3.00 (br d, J = 2.0 Hz, 1H), 2.97-2.96 |
| | | | | (m, 1H), 2.94 (br s, 2H), 2.89 (br s, 1H), 2.85 (br s, 1H), 2.83- |
| | | | | 2.79 (m, 1H), 2.63 (br s, 1H), 2.54 (br s, 1H), 2.29 (br s, |
| | | | | 1H), 2.26 (br s, 1H), 2.24-2.20 (m, 2H), 2.20-2.16 (m, 1H), |
| | | | | 2.01 (br s, 1H), 1.99-1.95 (m, 1H), 1.94-1.85 (m, 3H), 1.76 |
| | | | | (br d, J = 11.8 Hz, 3H), 1.67 (br s, 3H), 1.60 (br d, J = 2.1 |
| | | | | Hz, 1H), 1.58-1.49 (m, 2H), 1.47-1.41 (m, 2H), 1.38 (br d, |
| | | | | J = 15.8 Hz, 2H), 1.16-1.09 (m, 1H), 0.94 (s, 9H) |
| I-254 | OC | HI | 930.7 | 0.93 (br s, 9 H) 1.09-1.50 (m, 8 H) 1.71-1.94 (m, 9 H) 2.04 |
| | | | | (br s, 4 H) 2.14-2.33 (m, 10 H) 2.86 (br s, 4 H) 3.02 (br d, |
| | | | | J = 5.13 Hz, 2 H) 3.16 (br s, 2 H) 3.63-3.69 (m, 2 H) 4.01- |
| | | | | 4.05 (m, 1 H) 4.12-4.21 (m, 2 H) 4.36 (br dd, J = 14.01, 5.38 |
| | | | | Hz, 5 H) 5.10 (br d, J = 2.13 Hz, 1H) 5.84 (br d, J = 8.50 Hz, 1 |
| | | | | H) 6.79-6.90 (m, 2 H) 7.20 (br s, 2 H) 7.27-7.40 (m, 5 H) |
| | | | | 7.92 (br d, J = 6.50 Hz, 1 H) 8.49-8.58 (m, 1 H) |
| I-255 | OC | HI | 930.7 | 0.93 (s, 9 H) 1.23 (br s, 1 H) 1.33-1.56 (m, 11 H) 1.75 (br |
| | | | | s, 3H) 1.84-2.03 (m, 6 H) 2.16-2.33 (m, 10 H) 2.67 (br s, |
| | | | | 1 H) 2.80-2.87 (m, 1 H) 2.94-3.06 (m, 4 H) 3.16 (br d, |
| | | | | J = 8.38 Hz, 2 H) 3.47 (br d, J = 3.63 Hz, 1 H) 3.61-3.73 (m, |
| | | | | 2 H) 4.04 (br d, J = 11.38 Hz, 1 H) 4.10-4.23 (m, 2 H) 4.32- |
| | | | | 4.45 (m, 4 H) 5.11 (br d, J = 3.00 Hz, 1 H) 5.83 (br d, J = 9.13 |
| | | | | Hz, 1 H) 6.80-6.88 (m, 2 H) 7.17-7.22 (m, 2 H) 7.29-7.39 |
| | | | | (m, 5 H) 7.92 (br d, J = 7.50 Hz, 1 H) 8.54 (br t, J = 6.00 Hz, 1 H) |
| I-256 | NP | HI | 930.4 | 8.53 (t, J = 5.60 Hz, 1 H), 8.31 (s, 1 H), 7.91 (d, J = 7.20 Hz, 1 |
| | | | | H), 7.27-7.42 (m, 5 H), 7.15-7.23 (m, 2 H), 6.79-6.91 (m, |
| | | | | 2 H), 5.59 (d, J = 8.80 Hz, 1 H), 4.96-5.19 (m, 1 H), 4.30- |
| | | | | 4.48 (m, 4 H), 4.09-4.23 (m, 2 H), 3.97-4.07 (m, 1 H), 3.83 |
| | | | | (d, J = 12.40 Hz, 1 H), 3.64-3.74 (m, 3 H), 3.03 (d, J = 8.20 |
| | | | | Hz, 3 H), 2.86-2.95 (m, 3 H), 2.74-2.84 (m, 3 H), 2.67 (s, |
| | | | | 1 H), 2.24-2.35 (m, 3 H), 2.08-2.22 (m, 5 H), 2.01 (dd, |
| | | | | J = 12.00, 7.20 Hz, 2 H), 1.83-1.95 (m, 3 H), 1.53-1.77 (m, |
| | | | | 8H), 1.27-1.46 (m, 5 H), 1.03-1.14 (m, 1 H), 0.94 (s, 9 H) |
| I-259 | FJ | YZ | 964.8 | 12.45 (br s, 1H), 8.72 (s, 1H), 8.37 (br d, J = 8.0 Hz, 1H), |
| | | | | 8.34 (s, 2H), 8.23-8.20 (m, 1H), 8.16 (s, 1H), 7.42 (d, J = |
| | | | | 8.4 Hz, 2H), 7.32-7.27 (m, 3H), 6.99-6.94 (m, 2H), 6.04 |
| | | | | (br d, J = 6.4 Hz, 1H), 5.85 (br d, J = 9.6 Hz, 1H), 5.12-5.02 |
| | | | | (m, 2H), 4.87 (br t, J = 7.2 Hz, 1H), 4.47 (br s, 1H), 4.41- |
| | | | | 4.33 (m, 2H), 4.26 (br s, 1H), 4.13 (s, 1H), 3.88-3.82 (m, |
| | | | | 2H), 3.77-3.71 (m, 2H), 3.59 (br s, 2H), 3.48-3.44 (m, 3H), |
| | | | | 2.96-2.85 (m, 4H), 2.59 (br d, J = 6.8 Hz, 2H), 2.30-2.23 |
| | | | | (m, 2H), 2.04-1.90 (m, 4H), 1.82-1.68 (m, 6H), 1.65-1.57 |
| | | | | (m, 3H), 1.54 (d, J = 6.4 Hz, 3H), 1.33 (d, J = 7.2 Hz, 4H), |
| | | | | 0.97-0.89 (m, 6H) |
| I-262^{e} | FJ | TX | 1008.6 | 0.92 (s, 3 H) 0.96 (s, 3 H) 1.49-1.58 (m, 5 H) 1.63 (br dd, |
| | | | | J = 13.2, 7.6 Hz, 3 H) 1.72 (br d, J = 11.2 Hz, 2 H) 1.78-1.92 |
| | | | | (m, 4 H) 1.93-2.05 (m, 4 H) 2.27 (br s, 2 H) 2.35-2.42 (m, |
| | | | | 1 H) 2.89 (br d, J = 12.01 Hz, 4 H) 3.37-3.45 (m, 2 H) 3.49 |
| | | | | (br t, J = 7.2 Hz, 3 H) 3.65 (br s, 2 H) 3.72 (br d, J = 8.4 Hz, 1 |
| | | | | H) 3.78 (br d, J = 8.4 Hz, 1H) 3.83 (br d, J = 8.0 Hz, 1 H) 3.88 |
| | | | | (br s, 1 H) 3.95 (s, 2H) 4.12 (s, 1 H) 4.20 (br dd, J = 15.6, 5.25 |
| | | | | Hz, 2 H) 4.34-4.40 (m, 4 H) 5.02-5.07 (m, 1 H) 5.77 (br d, |
| | | | | J = 9.2 Hz, 1 H) 6.01-6.06 (m, 1 H) 6.97 (d, J = 7.6 Hz, 2 H) |
| | | | | 7.31 (d, J = 8.0 Hz, 3 H) 7.37-7.41 (m, 2 H) 8.19 (br d, J = 9.6 |
| | | | | Hz, 1H) 8.33 (s, 2H) 8.53 (br t, J = 5.6 Hz, 1 H) 8.71 (s, 1 H) |
| | | | | 12.45 (br s, 1 H) |
| I-263^{e} | FJ | TV | 1008.8 | 1.00 (s, 6 H) 1.49-1.65 (m, 7 H) 1.66-1.89 (m, 8 H) 1.92 |
| | | | | (br s, 5 H) 2.31-2.40 (m, 1 H) 2.83 (br d, J = 9.2 Hz, 2 H) |
| | | | | 2.90-2.99 (m, 2 H) 3.40 (br t, J = 9.2 Hz, 1 H) 3.47-3.54 (m, |
| | | | | 4 H) 3.60 (br d, J = 7.6 Hz, 1 H) 3.65 (br d, J = 8.4 Hz, 1 H) |
| | | | | 3.76 (br d, J = 8.0 Hz, 1 H) 3.84 (br dd, J = 10.0, 5.2 Hz, 1 H) |
| | | | | 3.93 (s, 2 H) 4.12 (s, 1 H) 4.16-4.23 (m, 2 H) 4.28-4.42 |
| | | | | (m, 4 H) 5.02-5.08 (m, 1 H) 5.93 (br d, J = 7.6 Hz, 1 H) 6.04 |
| | | | | (q, J = 6.4 Hz, 1 H) 6.94-6.99 (m, 2 H) 7.22-7.32 (m, 3 H) |
| | | | | 7.41 (d, J = 8.0 Hz, 2 H) 8.19-8.24 (m, 2 H) 8.33 (s, 2 H) 8.71 |
| | | | | (s, 1 H) 12.47 (br s, 1 H) |
| I-268 | QE | HI | 975.7 | 14.56-14.05 (m, 1H), 12.81-12.30 (m, 1H), 8.73-8.67 (m, |
| | | | | 1H), 8.60-8.52 (m, 1H), 8.42 (s, 1H), 8.36-8.32 (m, 2H), |
| | | | | 8.22-8.18 (m, 1H), 7.38-7.26 (m, 5H), 6.99-6.93 (m, 2H), |
| | | | | 6.52-6.42 (m, 1H), 6.25 (d, J = 9.6 Hz, 1H), 6.04 (q, J = 6.4 |
| | | | | Hz, 1H), 5.08-5.01 (m, 1H), 4.58 (dd, J = 7.2, 10.4 Hz, 1H), |
| | | | | 4.44 (s, 1H), 4.42-4.29 (m, 5H), 4.28-4.15 (m, 3H), 4.14- |
| | | | | 4.11 (m, 1H), 3.84-3.71 (m, 2H), 3.47-3.37 (m, 2H), 3.33- |
| | | | | 3.24 (m, 2H), 3.23-3.16 (m, 1H), 2.92 (d, J = 3.2 Hz, 3H), |
| | | | | 2.78-2.69 (m, 1H), 2.46-2.38 (m, 1H), 2.15-2.04 (m, 1H), |
| | | | | 2.04-1.83 (m, 6H), 1.82-1.75 (m, 2H), 1.67-1.58 (m, 3H), |
| | | | | 1.54 (d, J = 6.4 Hz, 3H), 0.94-0.90 (m, 9H) |
| I-269 | QF | HI | 919.4 | 8.78 (s, 2H), 8.54 (t, J = 6.0 Hz, 1H), 7.96 (d, J = 8.0 Hz, |
| | | | | 1H), 7.63 (d, J = 8.0 Hz, 2H), 7.44-7.29 (m, 8H), 7.25-7.20 |
| | | | | (m, 1H), 6.91-6.84 (m, 2H), 5.86 (br d, J = 9.2 Hz, 1H), 5.11 |
| | | | | (d, J = 3.6 Hz, 1H), 4.85-4.78 (m, 2H), 4.45-4.31 (m, 4H), |
| | | | | 4.25 (br d, J = 13.2 Hz, 1H), 4.19 (dd, J = 6.0, 16.2 Hz, 1H), |
| | | | | 4.13 (s, 1H), 3.73-3.60 (m, 3H), 3.55-3.47 (m, 2H), 3.43- |
| | | | | 3.34 (m, 3H), 3.31-3.20 (m, 6H), 3.06-2.98 (m, 1H), 2.91- |
| | | | | 2.82 (m, 1H), 2.34 (br d, J = 4.4 Hz, 4H), 2.05-1.98 (m, |
| | | | | 1H), 1.92-1.84 (m, 1H), 0.98-0.90 (m, 8H) |
| I-271 | QH | KC | 964.9 | 14.34.14.31 (ₘ, 1H), 12.43 (s, 1H), 8.71 (s, 1H), 8.39-8.35 |
| | | | | (m, 1H), 8.33 (s, 2H), 8.21 (br d, J = 7.2 Hz, 1H), 7.32-7.25 |
| | | | | (m, 2H), 6.98-6.94 (m, 2H), 6.94 (s, 1H), 6.84 (d, J = 7.6 |
| | | | | Hz, 1H), 6.04 (q, J = 5.6 Hz, 1H), 5.67-5.62 (m, 1H), 5.10 |
| | | | | (d, J = 3.6 Hz, 1H), 5.08-5.02 (m, 1H), 4.42 (t, J = 8.1 Hz, |
| | | | | 1H), 4.35-4.29 (m, 2H), 4.26-4.18 (m, 1H), 4.15-4.08 (m, |
| | | | | 1H), 3.83 (br d, J = 8.0 Hz, 2H), 3.79 (s, 3H), 3.76 (s, 2H), |
| | | | | 3.62 (br s, 2H), 3.44-3.39 (m, 1H), 2.97-2.89 (m, 2H), 2.89- |
| | | | | 2.82 (m, 2H), 2.41-2.32 (m, 2H), 2.29-2.21 (m, 2H), 2.03 |
| | | | | (s, 3H), 2.01-1.98 (m, 1H), 1.94-1.86 (m, 3H), 1.77-1.68 |
| | | | | (m, 4H), 1.64-1.57 (m, 2H), 1.54 (d, J = 6.4 Hz, 3H), 0.90 |
| | | | | (s, 9H) |
| I-276 | QT | HI | 923.6 | 0.93 (s, 9H) 1.35 (br d, J = 3.00 Hz, 2H) 1.78-2.06 (m, 6 H) |
| | | | | 2.23 (br t, J = 10.19 Hz, 2 H) 2.30-2.42 (m, 2 H) 2.79-2.89 |
| | | | | (m, 3 H) 3.01-3.08 (m, 2 H) 3.13-3.24 (m, 4 H) 3.46 (br |
| | | | | dd, J = 8.19, 3.81 Hz, 2 H) 3.64 (br s, 2 H) 3.73-3.83 (m, 2 |
| | | | | H) 3.91-4.07 (m, 3 H) 4.12-4.23 (m, 2 H) 4.30-4.44 (m, |
| | | | | 4 H) 4.66 (br d, J = 13.51 Hz, 2 H) 5.12 (br d, J = 2.50 Hz, 1 H) |
| | | | | 5.71 (br d, J = 9.26 Hz, 1 H) 6.81-6.88 (m, 2 H) 7.17-7.23 |
| | | | | (m, 2H) 7.28-7.40 (m, 5H) 7.91 (br d, J = 7.13 Hz, 1 H) 8.26 |
| | | | | (s, 2 H) 8.54 (br t, J = 6.07 Hz, 1 H) |
| I-284 | RJ | HI | 952.7 | 8.54 (t, J = 6.0 Hz, 1H), 8.24 (s, 2H), 7.95 (d, J = 8.0 Hz, |
| | | | | 1H), 7.40 (d, J = 3.2 Hz, 1H), 7.39-7.36 (m, 2H), 7.33-7.29 |
| | | | | (m, 3H), 7.24-7.19 (m, 1H), 6.90-6.84 (m, 2H), 5.85 (d, J = |
| | | | | 9.2 Hz, 1H), 5.11 (d, J = 3.2 Hz, 1H), 4.62 (d, J = 12.4 Hz, |
| | | | | 2H), 4.43-4.36 (m, 3H), 4.35-4.32 (m, 1H), 4.22-4.15 (m, |
| | | | | 2H), 4.13 (s, 1H), 3.73-3.58 (m, 4H), 3.27-3.23 (m, 3H), |
| | | | | 3.11-3.02 (m, 2H), 3.01-2.95 (m, 3H), 2.91 (s, 2H), 2.70- |
| | | | | 2.64 (m, 3H), 2.19 (s, 4H), 2.06-1.93 (m, 4H), 1.91-1.85 |
| | | | | (m, 1H), 1.68 (d, J = 4.8 Hz, 2H), 1.62-1.53 (m, 4H), 0.94 |
| | | | | (s, 9H) |
| I-288 | FJ | RP | 950.4 | 14.32 (s, 1H), 12.62-12.25 (m, 1H), 8.71 (s, 1H), 8.53 (t, J = |
| | | | | 6.0 Hz, 1H), 8.33 (s, 2H), 8.22-8.19 (m, 1H), 7.40-7.36 |
| | | | | (m, 2H), 7.32-7.27 (m, 3H), 6.97 (d, J = 1.2 Hz, 1H), 6.96- |
| | | | | 6.94 (m, 1H), 6.06-6.01 (m, 1H), 5.96 (d, J = 6.4 Hz, 1H), |
| | | | | 5.11 (d, J = 3.6 Hz, 1H), 5.08-5.02 (m, 1H), 4.47 (t, J = 4.8 |
| | | | | Hz, 1H), 4.40-4.28 (m, 4H), 4.23-4.16 (m, 1H), 4.13 (s, |
| | | | | 1H), 3.90-3.81 (m, 2H), 3.79-3.72 (m, 2H), 3.69-3.62 (m, |
| | | | | 2H), 3.45 (d, J = 4.8 Hz, 2H), 3.41 (s, 1H), 3.11-3.03 (m, |
| | | | | 1H), 2.97-2.89 (m, 3H), 2.54 (s, 1H), 2.31 (s, 1H), 2.24 (dd, |
| | | | | J = 2.0, 4.0 Hz, 1H), 2.17 (dd, J = 0.8, 9.5 Hz, 1H), 2.13- |
| | | | | 2.07 (m, 1H), 2.06-1.97 (m, 2H), 1.87 (ddd, J = 3.8, 8.3, |
| | | | | 16.9 Hz, 2H), 1.80-1.71 (m, 2H), 1.68-1.58 (m, 2H), 1.57- |
| | | | | 1.52 (m, 4H), 1.41-1.34 (m, 1H), 1.27-1.15 (m, 1H), 0.95 |
| | | | | (s, 3H), 0.93-0.88 (m, 3H) |
| I-316 | FJ | TO | 984.4 | 14.33 (s, 1H), 8.71 (s, 1H), 8.33 (s, 2H), 8.22-8.18 (m, 2H), |
| | | | | 7.54 (d, J = 1.6 Hz, 1H), 7.42-7.27 (m, 3H), 6.98-6.93 (m, |
| | | | | 2H), 6.31 (br d, J = 7.6 Hz, 1H), 6.04 (br d, J = 6.4 Hz, 1H), |
| | | | | 5.17 (d, J = 4.0 Hz, 1H), 5.05 (br dd, J = 4.4, 12.8 Hz, 1H), |
| | | | | 4.69 (t, J = 4.8 Hz, 1H), 4.44-4.32 (m, 3H), 4.29 (s, 1H), |
| | | | | 4.17-4.08 (m, 2H), 3.88 (br dd, J = 5.2, 10.6 Hz, 1H), 3.68 |
| | | | | (br d, J = 8.0 Hz, 1H), 3.58-3.47 (m, 5H), 3.41-3.35 (m, |
| | | | | 3H), 2.98-2.89 (m, 2H), 2.85-2.76 (m, 2H), 2.40-2.34 (m, |
| | | | | 2H), 2.11-1.97 (m, 5H), 1.80-1.66 (m, 8H), 1.63-1.57 (m, |
| | | | | 2H), 1.54 (br d, J = 6.8 Hz, 3H), 0.99 (s, 6H) |
| I-317 | FJ | TP | 984.4 | 14.34 (s, 1H), 12.43 (d, J = 3.2 Hz, 1H), 8.72 (s, 1H), 8.64 (t, |
| | | | | J = 6.0 Hz, 1H), 8.34 (s, 2H), 8.24-8.18 (m, 1H), 7.58-7.50 |
| | | | | (m, 2H), 7.38-7.26 (m, 2H), 7.01-6.92 (m, 2H), 6.09-6.01 |
| | | | | (m, 1H), 5.95 (d, J = 9.6 Hz, 1H), 5.13 (d, J = 3.6 Hz, 1H), |
| | | | | 5.09-5.01 (m, 1H), 4.49-4.45 (m, 1H), 4.43-4.38 (m, 1H), |
| | | | | 4.38-4.32 (m, 3H), 4.30 (s, 1H), 4.27-4.20 (m, 1H), 3.91- |
| | | | | 3.81 (m, 2H), 3.79-3.70 (m, 2H), 3.70-3.61 (m, 2H), 3.49- |
| | | | | 3.37 (m, 4H), 2.98-2.90 (m, 2H), 2.86 (d, J = 10.4 Hz, 2H), |
| | | | | 2.55 (s, 1H), 2.41-2.37 (m, 1H), 2.26 (t, J = 8.8 Hz, 2H), |
| | | | | 2.07-2.01 (m, 1H), 1.95-1.87 (m, 3H), 1.78-1.68 (m, 4H), |
| | | | | 1.62-1.57 (m, 2H), 1.54 (d, J = 6.8 Hz, 3H), 1.41-1.32 (m, |
| | | | | 1H), 0.92 (d, J = 12.4 Hz, 6H) |
| I-318 | JL | TQ | 970.4 | 8.72-8.63 (m, 2H), 8.31 (d, J = 9.6 Hz, 3H), 8.14-8.07 (m, |
| | | | | 1H), 7.57-7.53 (m, 2H), 7.34-7.27 (m, 2H), 7.00-6.95 (m, |
| | | | | 2H), 6.03 (t, J = 5.8 Hz, 1H), 5.71-5.66 (m, 1H), 5.10-5.02 |
| | | | | (m, 1H), 4.39 (d, J = 6.4 Hz, 2H), 4.37-4.32 (m, 2H), 4.31- |
| | | | | 4.29 (m, 1H), 4.26-4.18 (m, 1H), 3.96 (d, J = 5.6 Hz, 1H), |
| | | | | 3.90-3.86 (m, 1H), 3.86-3.81 (m, 2H), 3.78 (d, J = 8.4 Hz, |
| | | | | 1H), 3.72 (d, J = 8.8 Hz, 1H), 3.64 (s, 2H), 3.61-3.54 (m, |
| | | | | 2H), 2.95-2.82 (m, 4H), 2.30-2.23 (m, 2H), 2.09-2.00 (m, |
| | | | | 2H), 1.96-1.88 (m, 3H), 1.78-1.67 (m, 4H), 1.64-1.53 (m, |
| | | | | 2H), 1.04-0.98 (m, 3H), 0.91 (s, 9H) |
| I-323 | UH | HI | 925.5 | 12.70-12.33 (m, 1H), 8.71 (s, 1H), 8.55 (t, J = 6.0 Hz, 1H), |
| | | | | 8.26-8.13 (m, 1H), 7.54-7.26 (m, 6H), 7.01-6.85 (m, 2H), |
| | | | | 5.64 (dd, J = 5.6, 9.2 Hz, 1H), 5.44-5.30 (m, 1H), 4.42- |
| | | | | 4.29 (m, 5H), 4.22-4.12 (m, 3H), 3.97-3.90 (m, 1H), 3.86 |
| | | | | (d, J = 8.0 Hz, 1H), 3.81 (d, J = 8.0 Hz, 1H), 3.79-3.76 (m, |
| | | | | 1H), 3.75-3.68 (m, 3H), 3.66-3.60 (m, 4H), 3.09 (ddd, J = |
| | | | | 6.4, 11.2, 17.2 Hz, 1H), 2.89 (dd, J = 3.6, 17.2 Hz, 1H), 2.78 |
| | | | | (d, J = 10.4 Hz, 1H), 2.62 (dd, J = 3.2, 8.0 Hz, 1H), 2.37 (ddd, |
| | | | | J = 3.2, 6.8, 9.2 Hz, 1H), 2.26-2.20 (m, 1H), 2.09-1.96 (m, |
| | | | | 2H), 1.95-1.83 (m, 5H), 1.77 (t, J = 10.8 Hz, 1H), 1.56 (d, J = |
| | | | | 6.4 Hz, 3H), 1.48 (d, J = 10.0 Hz, 1H), 0.91 (s, 9H) |
| I-335 | UX | HI | 925.6 | 12.49 (d, J = 1.2 Hz, 1H), 8.71 (s, 1H), 8.54 (t, J = 6.0 Hz, |
| | | | | 1H), 8.18-8.15 (m, 1H), 7.42-7.28 (m, 5H), 7.05-6.92 (m, |
| | | | | 2H), 6.87 (s, 1H), 5.65 (d, J = 9.6 Hz, 1H), 5.37 (q, J = 6.4 |
| | | | | Hz, 1H), 5.25-5.00 (m, 1H), 4.42-4.30 (m, 4H), 4.22-4.11 |
| | | | | (m, 3H), 3.89-3.80 (m, 2H), 3.79-3.69 (m, 3H), 3.63 (s, |
| | | | | 2H), 3.15-3.01 (m, 2H), 2.93-2.79 (m, 3H), 2.71-2.59 (m, |
| | | | | 2H), 2.25 (d, J = 9.2 Hz, 1H), 2.04-1.83 (m, 8H), 1.56 (d, J = |
| | | | | 6.4 Hz, 3H), 1.50 (d, J = 11.6 Hz, 1H), 1.27-1.17 (m, 2H), |
| | | | | 0.92 (s, 9H) |
| I-339ⁱ | FJ | VE | 1018 | 8.71 (s, 1H), 8.34 (s, 2H), 8.28 (br d, J = 3.2 Hz, 2H), 8.23- |
| | | | | 8.16 (m, 2H), 7.41 (d, J = 8.4 Hz, 2H), 7.32-7.28 (m, 1H), |
| | | | | 7.23 (d, J = 8.0 Hz, 2H), 6.99-6.94 (m, 2H), 6.07-6.01 (m, |
| | | | | 1H), 5.87 (br d, J = 8.0 Hz, 1H), 5.08-5.02 (m, 1H), 4.41- |
| | | | | 4.26 (m, 5H), 4.17 (br d, J = 4.8 Hz, 1H), 4.13 (s, 1H), 3.83 |
| | | | | (br dd, J = 5.2, 10.6 Hz, 2H), 3.76 (br d, J = 7.6 Hz, 2H), 3.66 |
| | | | | (br d, J = 8.8 Hz, 2H), 3.60 (br d, J = 8.0 Hz, 2H), 3.52-3.50 |
| | | | | (m, 2H), 3.39 (br s, 2H), 2.96-2.86 (m, 6H), 2.83 (br d, J = |
| | | | | 9.6 Hz, 2H), 2.45 (br d, J = 7.6 Hz, 3H), 2.36 (br s, 1H), 2.32 |
| | | | | (br s, 2H), 2.14-2.07 (m, 2H), 2.03 (br dd, J = 5.2, 7.8 Hz, |
| | | | | 1H), 1.98-1.92 (m, 1H), 1.86-1.78 (m, 2H), 1.76 (br d, J = |
| | | | | 6.8 Hz, 1H), 1.71 (br d, J = 12.4 Hz, 3H), 1.62-1.57 (m, 2H), |
| | | | | 1.54 (d, J = 6.4 Hz, 3H), 1.46-1.38 (m, 1H), 0.97 (br d, J = |
| | | | | 6.4 Hz, 6H) |
| I-340ⁱ | FJ | VG | 1018 | 8.71 (s, 1H), 8.56 (br t, J = 5.6 Hz, 1H), 8.34 (s, 2H), 8.27 (s, |
| | | | | 2H), 8.23-8.19 (m, 1H), 7.41-7.36 (m, 2H), 7.33-7.28 (m, |
| | | | | 3H), 6.99-6.94 (m, 2H), 6.04 (q, J = 6.4 Hz, 1H), 5.79 (br |
| | | | | d, J = 9.6 Hz, 1H), 5.08-5.02 (m, 1H), 4.44-4.32 (m, 5H), |
| | | | | 4.18 (br d, J = 5.2 Hz, 1H), 4.13 (s, 1H), 3.89 (br d, J = 8.0 |
| | | | | Hz, 1H), 3.82 (br d, J = 8.0 Hz, 1H), 3.78 (br d, J = 8.4 Hz, |
| | | | | 2H), 3.71 (br d, J = 8.8 Hz, 2H), 3.65 (br s, 3H), 3.41 (br s, |
| | | | | 2H), 3.30 (br s, 2H), 2.98-2.90 (m, 3H), 2.90-2.83 (m, 6H), |
| | | | | 2.43-2.37 (m, 3H), 2.26 (br d, J = 7.6 Hz, 4H), 2.05-1.98 |
| | | | | (m, 1H), 1.95-1.89 (m, 2H), 1.78 (br s, 1H), 1.75-1.69 (m, |
| | | | | 3H), 1.62-1.56 (m, 2H), 1.54 (d, J = 6.4 Hz, 3H), 1.40-1.32 |
| | | | | (m, 1H), 0.93 (br d, J = 13.2 Hz, 6H) |
| I-341^{j} | FJ | VM | 1062.7 | 12.54-12.40 (m, 1H), 8.74-8.66 (m, 1H), 8.59-8.49 (m, |
| | | | | 1H), 8.37-8.30 (m, 2H), 8.24-8.17 (m, 2H), 7.45-7.37 (m, |
| | | | | 2H), 7.34-7.23 (m, 3H), 6.99-6.93 (m, 2H), 6.08-6.00 (m, |
| | | | | 1H), 5.90-5.82 (m, 1H), 5.10-5.01 (m, 1H), 4.44-4.32 (m, |
| | | | | 4H), 4.25-4.12 (m, 2H), 4.11 (s, 1H), 3.91-3.88 (m, 1H), |
| | | | | 3.83-3.76 (m, 3H), 3.70 (s, 1H), 3.66 (s, 2H), 3.19-3.13 |
| | | | | (m, 3H), 3.04-3.00 (m, 1H), 2.96 (s, 5H), 2.82-2.74 (m, |
| | | | | 2H), 2.62-2.55 (m, 1H), 2.42-2.25 (m, 5H), 2.15-2.08 (m, |
| | | | | 1H), 2.01 (br t, J = 10.8 Hz, 2H), 1.97-1.88 (m, 2H), 1.90- |
| | | | | 1.83 (m, 1H), 1.82-1.69 (m, 4H), 1.65-1.58 (m, 2H), 1.54 |
| | | | | (d, J = 6.4 Hz, 4H), 1.42-1.31 (m, 1H), 1.02-0.85 (m, 6H) |
| I-342^{j} | FJ | VK | 1062.7 | 8.71 (s, 1H), 8.33 (s, 2H), 8.22-8.17 (m, 2H), 7.40 (d, J = |
| | | | | 8.0 Hz, 2H), 7.29 (t, J = 7.6 Hz, 1H), 7.22 (d, J = 8.0 Hz, 2H), |
| | | | | 6.98-6.93 (m, 2H), 6.03 (q, J = 6.8 Hz, 1H), 5.82 (br d, J = |
| | | | | 7.6 Hz, 1H), 5.05 (br dd, J = 4.4, 13.2 Hz, 1H), 4.39-4.28 |
| | | | | (m, 4H), 4.17-4.10 (m, 2H), 3.85-3.73 (m, 3H), 3.66-3.52 |
| | | | | (m, 5H), 3.09-2.90 (m, 6H), 2.84-2.77 (m, 3H), 2.68-2.62 |
| | | | | (m, 1H), 2.40-1.90 (m, 14H), 1.83-1.66 (m, 7H), 1.62- |
| | | | | 1.56 (m, 2H), 1.53 (br d, J = 6.4 Hz, 3H), 0.96 (br d, J = 10.0 |
| | | | | Hz, 6H) |
| I-343^{j} | FJ | VZ | 1064 | 14.49-14.16 (m, 1H), 12.45 (br s, 1H), 8.71 (s, 1H), 8.33 (s, |
| | | | | 2H), 8.23-8.20 (m, 1H), 8.19 (s, 1H), 7.40 (d, J = 8.0 Hz, |
| | | | | 2H), 7.32-7.27 (m, 1H), 7.23 (d, J = 8.0 Hz, 2H), 6.99-6.93 |
| | | | | (m, 2H), 6.04 (q, J = 6.4 Hz, 1H), 5.89 (br d, J = 8.0 Hz, 1H), |
| | | | | 5.08-5.01 (m, 1H), 4.40-4.23 (m, 5H), 4.17 (br d, J = 5.2 |
| | | | | Hz, 1H), 4.13 (s, 1H), 3.84-3.74 (m, 3H), 3.68-3.58 (m, |
| | | | | 4H), 3.12-3.02 (m, 5H), 2.97-2.88 (m, 3H), 2.82 (br s, 3H), |
| | | | | 2.76-2.70 (m, 1H), 2.23-2.01 (m, 8H), 1.99-1.89 (m, 2H), |
| | | | | 1.80 (br d, J = 9.2 Hz, 2H), 1.74-1.67 (m, 4H), 1.62-1.58 |
| | | | | (m, 2H), 1.54 (d, J = 6.4 Hz, 3H), 1.43-1.37 (m, 1H), 0.96 |
| | | | | (br d, J = 5.6 Hz, 6H) |
| I-344^{j} | FJ | WC | 1062.8 | 14.55-14.12 (m, 1H), 12.45 (s, 1H), 8.71 (s, 1H), 8.53 (br t, |
| | | | | J = 6.0 Hz, 1H), 8.33 (s, 2H), 8.20 (dd, J = 1.6, 8.5 Hz, 1H), |
| | | | | 8.16-8.14 (m, 1H), 7.40-7.36 (m, 2H), 7.32-7.27 (m, 3H), |
| | | | | 6.99-6.93 (m, 2H), 6.04 (q, J = 6.0 Hz, 1H), 5.74 (br d, J = |
| | | | | 9.2 Hz, 1H), 5.09-5.00 (m, 1H), 4.40-4.32 (m, 4H), 4.22- |
| | | | | 4.11 (m, 2H), 3.89 (br d, J = 7.6 Hz, 1H), 3.83-3.77 (m, 2H), |
| | | | | 3.71 (br d, J = 8.4 Hz, 1H), 3.64 (br s, 2H), 3.10-3.00 (m, |
| | | | | 3H), 2.98-2.85 (m, 5H), 2.83-2.72 (m, 3H), 2.69-2.65 (m, |
| | | | | 1H), 2.34-2.22 (m, 5H), 2.13-2.01 (m, 3H), 2.01-1.92 (m, |
| | | | | 3H), 1.92-1.77 (m, 4H), 1.72 (br d, J = 9.6 Hz, 2H), 1.66- |
| | | | | 1.58 (m, 2H), 1.54 (br d, J = 6.4 Hz, 3H), 1.39-1.31 (m, 1H), |
| | | | | 0.92 (br d, J = 13.6 Hz, 6H) |
| I-369 | GT | YZ | 965.5 | 14.53-14.09 (m, 1H), 12.65-12.28 (m, 1H), 8.64 (s, 1H), |
| | | | | 8.36 (d, J = 7.6 Hz, 1H), 8.20 (s, 3H), 7.46-7.38 (m, 2H), |
| | | | | 7.33-7.25 (m, 3H), 7.01-6.89 (m, 2H), 6.03-5.94 (m, 1H), |
| | | | | 5.89-5.82 (m, 1H), 5.17-5.04 (m, 1H), 5.01-4.93 (m, 1H), |
| | | | | 4.91-4.81 (m, 1H), 4.53-4.43 (m, 1H), 4.42-4.31 (m, 2H), |
| | | | | 4.29-4.21 (m, 1H), 4.14-4.09 (m, 1H), 3.90-3.80 (m, 2H), |
| | | | | 3.78-3.69 (m, 2H), 3.64-3.55 (m, 2H), 3.50-3.43 (m, 3H), |
| | | | | 3.06-2.91 (m, 5H), 2.65-2.59 (m, 1H), 2.4 (s, 1H), 2.43- |
| | | | | 2.34 (m, 3H), 2.30-2.21 (m, 2H), 2.03-1.90 (m, 3H), 1.78- |
| | | | | 1.70 (m, 1H), 1.64 (s, 1H), 1.6 (s, 3H), 1.41-1.36 (m, 1H), |
| | | | | 1.36 (s, 3H), 1.08-0.98 (m, 1H), 0.98-0.93 (m, 1H), 0.94- |
| | | | | 0.88 (m, 3H) |
| I-410 | YH | HI | 934.6 | 12.44 (s, 1H), 8.71 (s, 1H), 8.60-8.48 (m, 1H), 8.33 (s, 2H), |
| | | | | 8.26-8.12 (m, 2H), 7.40-7.33 (m, 2H), 7.32-7.23 (m, 3H), |
| | | | | 7.04-6.84 (m, 2H), 6.03 (q, J = 6.4 Hz, 1H), 5.62 (d, J = 9.6 |
| | | | | Hz, 1H), 5.04 (dd, J = 5.2, 13.6 Hz, 1H), 4.42-4.28 (m, 4H), |
| | | | | 4.18 (dd, J = 5.6, 16.0 Hz, 1H), 4.12 (s, 1H), 3.91-3.67 (m, |
| | | | | 6H), 3.63 (d, J = 2.0 Hz, 3H), 2.96-2.87 (m, 4H), 2.33 (s, |
| | | | | 3H), 2.26-2.18 (m, 2H), 2.04-1.94 (m, 3H), 1.91-1.86 (m, |
| | | | | 1H), 1.83-1.75 (m, 2H), 1.72-1.59 (m, 4H), 1.53 (d, J = 6.4 |
| | | | | Hz, 3H), 0.95-0.88 (m, 9H) |
| I-416 | YT | HI | 948.9 | 14.12 (s, 1H), 12.47-12.41 (m, 1H), 8.71 (s, 1H), 8.57-8.50 |
| | | | | (m, 1H), 8.34 (s, 2H), 8.24 (s, 1H), 8.17-8.13 (m, 1H), 7.36 |
| | | | | (s, 2H), 7.30 (d, J = 8.0 Hz, 3H), 7.00-6.91 (m, 2H), 6.08- |
| | | | | 5.99 (m, 1H), 5.66-5.59 (m, 1H), 5.14-4.98 (m, 2H), 4.43- |
| | | | | 4.28 (m, 4H), 4.13 (s, 1H), 3.89-3.84 (m, 1H), 3.83-3.67 |
| | | | | (m, 4H), 3.63 (s, 2H), 3.02-2.91 (m, 4H), 2.70-2.62 (m, |
| | | | | 1H), 2.54 (s, 1H), 2.35-2.29 (m, 1H), 2.25-2.19 (m, 4H), |
| | | | | 2.12-2.06 (m, 1H), 1.99 (d, J = 5.6 Hz, 2H), 1.91-1.85 (m, |
| | | | | 1H), 1.79-1.72 (m, 3H), 1.70-1.59 (m, 3H), 1.54 (d, J = 6.4 |
| | | | | Hz, 5H), 0.91 (s, 9H) |

^{a}The coupling was run under standard conditions from 60-75° C. for 4-16 hrs. SFC or HPLC separation was performed with a variety of solvent conditions to separate the chiral compounds. Absolute stereochemistry of the compounds was assigned arbitrarily.
^{b}LC-MS (ESI⁺) m/z = (M/2 + H)⁺.
^{c}Dioxane was also added as a cosolvent. Then TFA was added to deprotect the coupled product by stirring at 60° C. for 9 hr.
^{d}The product of the coupling was further deprotected with CsF in DMSO at 50° C. for 1 hr. The final product was purified by HPLC.
^{e}The product of the coupling was further hydrolyzed with LiOH in THF, MeOH, and H₂O at rt for 12 hr. The final compound was purified by prep-HPLC.
^{f}The product of the coupling was further separated by SFC (column: Phenomenex-Cellulose-2 (250 mm × 30 mm, 10 um); mobile phase: [0.1% NH₃H₂O MEOH]; B %: 80%-80%, 13; 70 min) to give the diastereomers. Absolute stereochemistry was assigned arbitrarily.
^{g}Diastereomeric mixture at 3-prolidine.
^{h}Diastereomeric mixture at 3-piperidine.
ⁱThe product of the coupling was further the deprotected with TMSOTf and 2,6 lutidine in DCM at 0-25° C. for 2 hrs.
^{j}The product of the coupling was the deprotected with TMSOTf and 2,6 lutidine in DCM at 0 C. for 0.5 hrs. This material was then further deprotected with LiOH•H₂O in THF/H₂O at rt for 2 hrs.
^{k}Product of the coupling was further deprotected with FA in DMSO at rt for 1 hr.

TABLE 6B
Compounds synthesized via Method 4, using the corresponding amines and carbamates for the coupling.

| | | | LCMS (ESI+) | |
|---|---|---|---|---|
| I-#^{a} | Amine | Carbamate | m/z (M + H)⁺ | ¹H NMR (400 MHz, DMSO-d6) |
| I-272 | QI | QK | 735.2 | 8.95-8.88 (m, 1H), 8.42 (s, 1H), 8.32 (d, J = 8.0 Hz, 1H), |
| | | | | 7.94-7.91 (m, 1H), 7.50-7.31 (m, 4H), 7.25-7.19 (m, |
| | | | | 4H), 6.89-6.82 (m, 2H), 6.19-6.09 (m, 1H), 4.84 (t, J = |
| | | | | 7.2 Hz, 1H), 4.41 (t, J = 8.0 Hz, 1H), 4.35-4.21 (m, 4H), |
| | | | | 4.16-4.11 (m, 1H), 4.05 (d, J = 11.1 Hz, 1H), 3.75 (d, J = |
| | | | | 8.4 Hz, 1H), 3.57-3.53 (m, 1H), 3.50-3.44 (m, 3H), 3.19- |
| | | | | 3.15 (m, 1H), 3.10 (d, J = 10.8 Hz, 2H), 2.94-2.86 (m, |
| | | | | 1H), 2.81-2.73 (m, 2H), 2.28-2.22 (m, 2H), 2.06-2.00 |
| | | | | (m, 1H), 1.92-1.85 (m, 2H), 1.76-1.70 (m, 1H), 1.42 (d, |
| | | | | J = 6.8 Hz, 1H), 1.31 (d, J = 7.2 Hz, 3H), 0.96 (d, J = 6.4 Hz, |
| | | | | 3H), 0.85-0.82 (m, 3H), 0.77 (s, 1H), 0.77-0.75 (m, 1H) |
| I-273 | QI | QK | 735.2 | 8.80 (d, J = 7.6 Hz, 1H), 8.41-8.38 (m, 1H), 7.93 (d, J = |
| | | | | 8.0 Hz, 1H), 7.45-7.40 (m, 2H), 7.35 (d, J = 3.2 Hz, 1H), |
| | | | | 7.28 (d, J = 8.0 Hz, 2H), 7.25-7.19 (m, 2H), 6.88-6.83 |
| | | | | (m, 2H), 6.13-5.95 (m, 1H), 4.92-4.62 (m, 1H), 4.38- |
| | | | | 4.24 (m, 4H), 4.16-4.11 (m, 1H), 4.06 (d, J = 12.0 Hz, 1H), |
| | | | | 3.73-3.61 (m, 2H), 3.51-3.41 (m, 3H), 3.18 (d, J = 11.2 |
| | | | | Hz, 1H), 3.10 (d, J = 10.8 Hz, 2H), 2.95-2.86 (m, 1H), 2.81- |
| | | | | 2.73 (m, 2H), 2.28-2.20 (m, 2H), 2.05-1.97 (m, 1H), |
| | | | | 1.88 (t, J = 10.4 Hz, 1H), 1.78-1.70 (m, 1H), 1.43-1.31 |
| | | | | (m, 3H), 0.99-0.92 (m, 3H), 0.83-0.77 (m, 3H) |
| I-281 | RC | KC | 970.1 | 12.55-12.31 (m, 1H), 8.71 (s, 1H), 8.33 (s, 2H), 8.22-8.19 |
| | | | | (m, 2H), 7.29 (t, J = 8.0 Hz, 1H), 7.00 (s, 1H), 6.98-6.93 |
| | | | | (m, 2H), 6.86 (s, 1H), 6.04 (q, J = 6.4 Hz, 1H), 5.53 (br d, J = |
| | | | | 9.2 Hz, 1H), 5.13-4.98 (m, 2H), 4.42-4.33 (m, 2H), |
| | | | | 4.31-4.21 (m, 4H), 3.87-3.79 (m, 2H), 3.76-3.67 (m, |
| | | | | 2H), 3.64-3.54 (m, 2H), 3.41-3.36 (m, 2H), 3.00-2.89 |
| | | | | (m, 2H), 2.84 (br d, J = 10.4 Hz, 2H), 2.59-2.54 (m, 2H), |
| | | | | 2.40-2.31 (m, 1H), 2.25 (br d, J = 8.8 Hz, 2H), 1.96-1.85 |
| | | | | (m, 4H), 1.78-1.66 (m, 4H), 1.64-1.56 (m, 2H), 1.53 (d, |
| | | | | J = 6.4 Hz, 3H), 0.89 (s, 9H) |
| I-287 | KV | SE | 981.7 | 8.52 (t, J = 6.0 Hz, 1H), 8.29-8.16 (m, 3H), 7.94 (d, J = 8.0 |
| | | | | Hz, 1H), 7.80-7.70 (m, 1H), 7.38 (d, J = 8.4 Hz, 3H), 7.34- |
| | | | | 7.28 (m, 3H), 7.21 (s, 1H), 6.85 (d, J = 8.0 Hz, 2H), 5.10 |
| | | | | (d, J = 4.4 Hz, 1H), 4.62 (d, J = 12.4 Hz, 2H), 4.54 (d, J = |
| | | | | 9.2 Hz, 1H), 4.42-4.29 (m, 4H), 4.23-4.15 (m, 2H), 4.12 |
| | | | | (s, 1H), 3.67-3.58 (m, 3H), 3.43 (s, 3H), 3.25 (s, 1H), 3.02 |
| | | | | (s, 7H), 2.67 (d, J = 10.8 Hz, 2H), 2.41-2.32 (m, 4H), 1.99 |
| | | | | (d, J = 8.4 Hz, 2H), 1.87 (d, J = 8.4 Hz, 1H), 1.84-1.77 (m, |
| | | | | 1H), 1.75-1.68 (m, 1H), 1.57-1.39 (m, 8H), 1.34-1.22 |
| | | | | (m, 3H), 0.94 (d, J = 18.0 Hz, 6H) |
| I-291^{d} | QI | RX | 954.6 | 10.26-9.53 (m, 2H), 9.24-8.56 (m, 1H), 8.39-8.16 (m, |
| | | | | 1H), 7.64-7.55 (m, 1H), 7.50 (d, J = 7.2 Hz, 1H), 7.64- |
| | | | | 7.48 (m, 1H), 7.46 (s, 1H), 7.44 (s, 1H), 7.43 (s, 1H), 7.41 |
| | | | | (s, 1H), 7.39 (s, 1H), 7.35 (s, 1H), 7.34-7.19 (m, 4H), 7.08- |
| | | | | 6.96 (m, 1H), 6.35-6.00 (m, 1H), 5.09-4.76 (m, 2H), |
| | | | | 4.46-4.25 (m, 4H), 4.21-4.05 (m, 2H), 3.88-3.75 (m, |
| | | | | 6H), 3.28 (s, 4H), 3.12-2.91 (m, 5H), 2.31-2.22 (m, 2H), |
| | | | | 2.14-1.92 (m, 8H), 1.83-1.59 (m, 4H), 1.55-1.38 (m, |
| | | | | 4H), 1.38-1.29 (m, 3H), 0.99-0.93 (m, 3H), 0.87-0.75 |
| | | | | (m, 3H) |
| I-299 | KV | KR | 981.7 | 8.52 (t, J = 6.0 Hz, 1H), 8.24 (s, 2H), 7.97-7.90 (m, 1H), |
| | | | | 7.80-7.74 (m, 1H), 7.38 (d, J = 8.4 Hz, 3H), 7.33-7.29 |
| | | | | (m, 3H), 7.21 (s, 1H), 6.90-6.82 (m, 2H), 5.20-5.02 (m, |
| | | | | 1H), 4.62 (d, J = 12.4 Hz, 2H), 4.56-4.51 (m, 1H), 4.42- |
| | | | | 4.30 (m, 4H), 4.12 (s, 3H), 3.61 (t, J = 3.6, 7.1 Hz, 3H), 3.45- |
| | | | | 3.40 (m, 3H), 3.02 (s, 7H), 2.76-2.60 (m, 3H), 2.39-2.27 |
| | | | | (m, 5H), 2.02-1.93 (m, 2H), 1.90-1.78 (m, 2H), 1.72 (d, |
| | | | | J = 11.2 Hz, 1H), 1.64-1.57 (m, 1H), 1.55-1.49 (m, 3H), |
| | | | | 1.47-1.39 (m, 4H), 1.34-1.25 (m, 3H), 0.96 (s, 3H), 0.92 |
| | | | | (s, 3H) |
| I-301 | HH | SL | 922.5 | 0.88-0.96 (m, 9 H) 1.30-1.40 (m, 2H) 1.85 (br dd, J = 12.4, |
| | | | | 8.8 Hz, 3 H) 1.94-2.11 (m, 6 H) 2.16-2.29 (m, 4 H) 2.31- |
| | | | | 2.41 (m, 3 H) 2.85 (br t, J = 12.0 Hz, 3 H) 3.05 (br d, J = 6.4 |
| | | | | Hz, 2 H) 3.16 (br d, J = 7.2 Hz, 3 H) 3.47 (br d, J = 4.0 Hz, 1 |
| | | | | H) 3.66 (br s, 2 H) 4.05 (br d, J = 11.6 Hz, 1 H) 4.13-4.21 |
| | | | | (m, 2 H) 4.35 (br s, 1 H) 4.37-4.44 (m, 2 H) 4.52 (br d, |
| | | | | J = 9.2 Hz, 1 H) 4.66 (br d, J = 12.4 Hz, 2 H) 5.14 (br s, 1 H) |
| | | | | 6.81-6.88 (m, 2 H) 7.18-7.24 (m, 2 H) 7.30-7.39 (m, 5 |
| | | | | H) 7.69 (br d, J = 9.2 Hz, 1 H) 7.92 (br d, J = 7.6 Hz, 1 H) 8.24 |
| | | | | (s, 2 H) 8.55 (br t, J = 5.6 Hz, 1 H) |
| I-302 | HH | SM | 922.5 | 0.93 (br s, 9 H) 1.32-1.39 (m, 2 H) 1.80-2.02 (m, 10 H) |
| | | | | 2.07-2.23 (m, 7 H) 2.68 (br s, 2 H) 2.83 (br d, J = 10.4 Hz, |
| | | | | 3 H) 3.05 (br s, 2 H) 3.17 (br s, 3 H) 3.45-3.47 (m, 1 H) |
| | | | | 3.65 (br s, 2 H) 4.03-4.08 (m, 1 H) 4.13-4.21 (m, 2 H) |
| | | | | 4.34 (br s, 1 H) 4.37-4.43 (m, 2 H) 4.53 (br d, J = 9.2 Hz, 1 |
| | | | | H)4.61-4.68 (m, 2 H) 5.10-5.15 (m, 1 H) 6.80-6.90 (m, |
| | | | | 2 H) 7.22 (br d, J = 0.8 Hz, 2 H) 7.31-7.42 (m, 5 H) 7.65- |
| | | | | 7.72 (m, 1 H) 7.87-7.95 (m, 1 H) 8.24 (br s, 2 H) 8.51- |
| | | | | 8.59 (m, 1 H) |
| I-311 | ST | OH | 1014.8 | 8.64 (t, J = 5.6 Hz, 1H), 8.24 (s, 3H), 7.95 (d, J = 7.2 Hz, |
| | | | | 1H), 7.83-7.75 (m, 1H), 7.58-7.54 (m, 1H), 7.44-7.38 |
| | | | | (m, 1H), 7.36-7.30 (m, 2H), 7.25-7.19 (m, 1H), 6.90- |
| | | | | 6.83 (m, 2H), 4.63 (d, J = 12.4 Hz, 2H), 4.55 (d, J = 9.6 Hz, |
| | | | | 1H), 4.47-4.38 (m, 2H), 4.36 (d, J = 5.6 Hz, 2H), 4.29 (s, |
| | | | | 1H), 4.25 (d, J = 5.6 Hz, 1H), 4.22-4.16 (m, 2H), 3.70- |
| | | | | 3.57 (m, 4H), 3.42 (t, J = 7.2 Hz, 2H), 3.35-3.23 (m, 2H), |
| | | | | 3.13-2.91 (m, 6H), 2.75-2.64 (m, 2H), 2.56-2.54 (m, |
| | | | | 1H), 2.43-2.26 (m, 4H), 2.09-1.94 (m, 2H), 1.90 (d, J = |
| | | | | 4.4 Hz, 1H), 1.80 (d, J = 6.8 Hz, 1H), 1.72 (d, J = 11.6 Hz, |
| | | | | 1H), 1.58-1.35 (m, 8H), 1.35-1.21 (m, 3H), 0.93 (d, J = |
| | | | | 16 Hz, 6H) |
| I-312 | ST | OE | 1014.8 | 8.63 (t, J = 5.6 Hz, 1H), 8.29-8.18 (m, 2H), 7.99-7.91 (m, |
| | | | | 1H), 7.79 (d, J = 9.2 Hz, 1H), 7.60-7.51 (m, 2H), 7.40 (s, |
| | | | | 1H), 7.37-7.29 (m, 2H), 7.26-7.18 (m, 1H), 6.91-6.83 |
| | | | | (m, 2H), 4.63 (d, J = 12.4 Hz, 2H), 4.54 (d, J = 9.6 Hz, 1H), |
| | | | | 4.47-4.38 (m, 2H), 4.35 (d, J = 5.6 Hz, 2H), 4.29 (s, 1H), |
| | | | | 4.25 (d, J = 5.6 Hz, 1H), 4.21 (d, J = 4.4 Hz, 1H), 4.18 (s, |
| | | | | 1H), 3.68-3.58 (m, 5H), 3.43 (t, J = 7.2 Hz, 3H), 3.35- |
| | | | | 3.27 (m, 2H), 3.27-3.22 (m, 1H), 3.08-2.94 (m, 5H), 2.73- |
| | | | | 2.64 (m, 2H), 2.55 (s, 1H), 2.38 (s, 3H), 2.08-1.95 (m, |
| | | | | 2H), 1.93-1.85 (m, 1H), 1.81 (s, 1H), 1.73 (d, J = 11.6 Hz, |
| | | | | 1H), 1.63 (d, J = 10.8 Hz, 1H), 1.54-1.41 (m, 6H), 1.33- |
| | | | | 1.23 (m, 3H), 1.18 (d, J = 6.4 Hz, 1H), 0.93 (d, J = 16 Hz, 6H) |
| I-328 | US | OM | 991.6 | 8.21-8.17 (m, 2H), 7.92 (d, J = 7.2 Hz, 1H), 7.83 (t, J = 4.8 |
| | | | | Hz, 1H), 7.57 (br d, J = 9.2 Hz, 1H), 7.38-7.33 (m, 1H), |
| | | | | 7.23-7.15 (m, 3H), 7.09 (s, 1H), 6.87-6.81 (m, 2H), 5.21- |
| | | | | 4.91 (m, 1H), 4.47-4.39 (m, 2H), 4.37-4.32 (m, 2H), |
| | | | | 4.29 (br dd, J = 4.4, 8.8 Hz, 2H), 4.06-4.01 (m, 1H), 3.83 |
| | | | | (s, 3H), 3.61 (br d, J = 4.4 Hz, 2H), 3.54 (br s, 2H), 3.17- |
| | | | | 3.15 (m, 2H), 3.04 (br d, J = 9.2 Hz, 2H), 2.93 (br d, J = |
| | | | | 12.4 Hz, 3H), 2.79 (br s, 2H), 2.31-2.17 (m, 4H), 2.13 (br |
| | | | | d, J = 8.8 Hz, 4H), 2.00-1.92 (m, 4H), 1.91 (br s, 3H), 1.80- |
| | | | | 1.62 (m, 9H), 1.44-1.33 (m, 4H), 0.88 (s, 9H) |
| I-329 | US | OE | 991.6 | 14.96-14.50 (m, 1H), 7.95-7.90 (m, 1H), 7.83 (t, J = 4.8 |
| | | | | Hz, 1H), 7.56 (br d, J = 8.8 Hz, 1H), 7.35 (br d, J = 3.6 Hz, |
| | | | | 1H), 7.24-7.15 (m, 3H), 7.10 (s, 1H), 6.88-6.82 (m, 2H), |
| | | | | 5.07 (d, J = 3.6 Hz, 1H), 4.50-4.24 (m, 6H), 4.04 (br d, J = |
| | | | | 11.2 Hz, 1H), 3.83 (s, 3H), 3.68-3.53 (m, 2H), 3.49- |
| | | | | 3.44 (m, 1H), 3.20-3.14 (m, 2H), 3.07-3.00 (m, 2H), 2.93- |
| | | | | 2.82 (m, 3H), 2.75 (br t, J = 10.0 Hz, 2H), 2.32-2.19 (m, |
| | | | | 2H), 2.18-2.01 (m, 8H), 1.95-1.82 (m, 5H), 1.79-1.53 |
| | | | | (m, 9H), 1.42-1.29 (m, 4H), 0.93-0.86 (m, 9H) |
| I-330 | UT | OM | 958.8 | 8.36-8.30 (m, 2H), 8.17 (d, J = 1.6 Hz, 1H), 8.10 (br t, J = |
| | | | | 5.2 Hz, 1H), 7.94-7.89 (m, 1H), 7.60-7.54 (m, 1H), 7.49 |
| | | | | (d, J = 1.6 Hz, 1H), 7.34 (br d, J = 3.2 Hz, 1H), 7.23-7.19 |
| | | | | (m, 2H), 6.87-6.81 (m, 2H), 4.49-4.43 (m, 3H), 4.42 (s, |
| | | | | 1H), 4.31-4.27 (m, 1H), 4.23 (br d, J = 4.8 Hz, 1H), 4.02 |
| | | | | (br s, 1H), 3.85 (s, 3H), 3.58 (br d, J = 0.8 Hz, 3H), 3.18- |
| | | | | 3.14 (m, 4H), 3.07-3.02 (m, 3H), 2.91-2.86 (m, 3H), 2.80- |
| | | | | 2.74 (m, 3H), 2.16-2.08 (m, 6H), 2.00-1.92 (m, 7H), |
| | | | | 1.78-1.73 (m, 3H), 1.67-1.59 (m, 5H), 1.41-1.32 (m, |
| | | | | 4H), 0.89 (s, 9H) |
| I-331 | UT | OL | 958.8 | 8.27 (s, 2H), 8.17 (d, J = 1.2 Hz, 1H), 8.11 (br t, J = 5.2 Hz, |
| | | | | 1H), 7.92 (br d, J = 7.2 Hz, 1H), 7.60-7.54 (m, 1H), 7.49 |
| | | | | (d, J = 1.2 Hz, 1H), 7.37-7.32 (m, 1H), 7.23-7.16 (m, 2H), |
| | | | | 6.88-6.80 (m, 2H), 4.51-4.43 (m, 3H), 4.42 (s, 1H), 4.31- |
| | | | | 4.27 (m, 1H), 4.24-4.18 (m, 1H), 4.03 (br d, J = 10.8 Hz, |
| | | | | 1H), 3.85 (s, 3H), 3.63 (br d, J = 4.4 Hz, 3H), 3.18-3.15 |
| | | | | (m, 2H), 3.13-3.07 (m, 2H), 3.04 (br d, J = 9.2 Hz, 3H), |
| | | | | 2.93-2.85 (m, 3H), 2.84-2.73 (m, 3H), 2.31-2.25 (m, |
| | | | | 1H), 2.22-2.15 (m, 2H), 2.15-2.05 (m, 6H), 1.99-1.90 |
| | | | | (m, 4H), 1.76 (br d, J = 9.6 Hz, 3H), 1.69-1.60 (m, 5H), |
| | | | | 1.42-1.30 (m, 4H), 0.89 (s, 9H) |
| I-364 | KV | OL | 957.6 | 8.54 (t, J = 6.0 Hz, 1H), 8.24 (s, 2H), 7.98-7.86 (m, 1H), |
| | | | | 7.72 (d, J = 9.6 Hz, 1H), 7.43-7.29 (m, 5H), 7.25-7.14 |
| | | | | (m, 2H), 6.91-6.77 (m, 2H), 4.55 (d, J = 9.6 Hz, 1H), 4.42- |
| | | | | 4.30 (m, 3H), 4.21 (d, J = 5.6 Hz, 1H), 4.17 (d, J = 5.6 Hz, |
| | | | | 1H), 4.14-4.12 (m, 1H), 4.04 (d, J = 10.4 Hz, 1H), 3.64 (s, |
| | | | | 2H), 3.18-3.15 (m, 2H), 3.14-3.09 (m, 2H), 3.07-2.99 |
| | | | | (m, 3H), 2.93 (d, J = 10.0 Hz, 2H), 2.88-2.73 (m, 4H), 2.30 |
| | | | | (s, 1H), 2.28-2.19 (m, 3H), 2.18-2.07 (m, 6H), 2.06 (s, |
| | | | | 1H), 2.02-1.91 (m, 3H), 1.90-1.83 (m, 2H), 1.78 (d, J = |
| | | | | 9.6 Hz, 3H), 1.69 (d, J = 9.2 Hz, 5H), 1.53-1.46 (m, 1H), |
| | | | | 1.44-1.35 (m, 4H), 0.93 (d, J = 18 Hz, 6H) |
| I-365 | KV | OM | 957.1 | 8.54 (t, J = 6.0 Hz, 1H), 8.41-8.12 (m, 1H), 7.92 (d, J = 7.6 |
| | | | | Hz, 1H), 7.76-7.68 (m, 1H), 7.42-7.28 (m, 5H), 7.25- |
| | | | | 7.16 (m, 2H), 6.84 (d, J = 8.0 Hz, 2H), 5.21-5.02 (m, 1H), |
| | | | | 4.55 (d, J = 9.6 Hz, 1H), 4.41-4.31 (m, 3H), 4.23-4.11 |
| | | | | (m, 2H), 4.08-4.00 (m, 1H), 3.64 (s, 2H), 3.18-3.15 (m, |
| | | | | 2H), 3.14-3.08 (m, 3H), 3.08-3.00 (m, 3H), 2.90 (d, J = |
| | | | | 10.8 Hz, 3H), 2.85-2.73 (m, 3H), 2.32-2.26 (m, 1H), 2.21- |
| | | | | 2.07 (m, 7H), 2.05-1.92 (m, 6H), 1.91-1.82 (m, 2H), |
| | | | | 1.80-1.71 (m, 3H), 1.69-1.56 (m, 5H), 1.43-1.32 (m, |
| | | | | 4H), 0.93 (br d, J = 17.6 Hz, 6H) |

^{a}The reaction was run anywhere from 0-40° C. for 10 min to 16 hrs. The coupled products were purified by prep-HPLC or SFC under variety of conditions. Absolute stereochemistry of the separated diastereomers and chirality on the spirocycles was assigned arbitrarily for all compounds. No SFC separation required for chiral coupling partners.
^{b}Cis-trans mixture at spirocycle.
^{c}No SFC separation. After coupling, HCl/dioxane at rt in DCM at rt was used to deprotect the amine. The final compound was purified by prep-HPLC. No SFC separation. Instead of HATU, T3P was used with DEA in DCM to couple at rt for 12 hrs.

### Example 5: Synthesis of (2S,4R)-4-hydroxy-1-(2-(3-(4-(4-(2-((S)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)butoxy)isoxazol-5-yl)-3-methylbutanoyl)-N-(4-(4-methylthiazol-5-yl)benzyl)pyrrolidine-2-carboxamide (I-50)

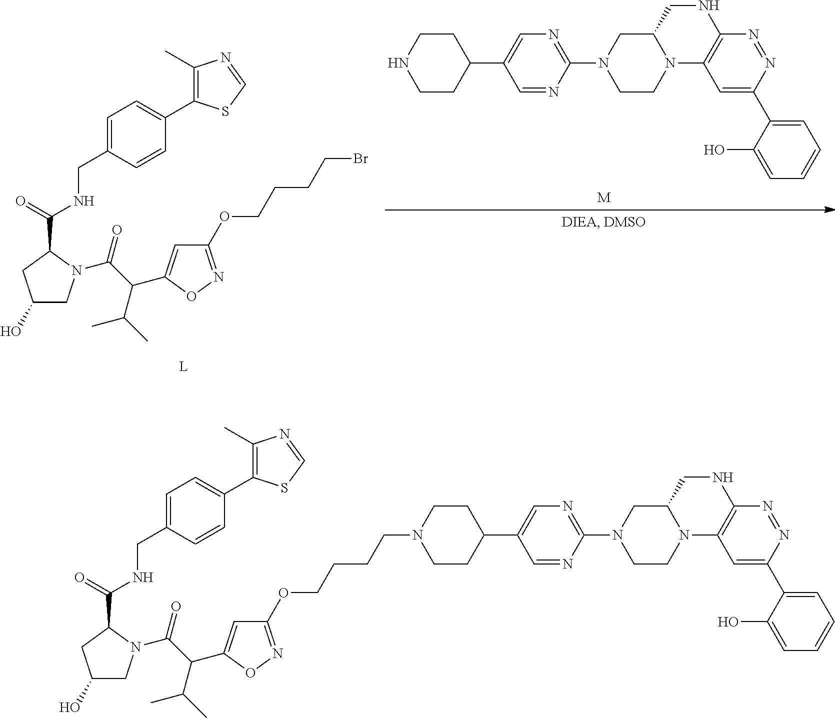

To a solution of (2S,4R)-1-[2-[3-(4-bromobutoxy)isoxazol-5-yl]-3-methyl-butanoyl]-4-hydroxy-N-[[4-(4-methylthiazol-5-yl)phenyl]methyl]pyrrolidine-2-carboxamide (30 mg, 48.4 umol, Intermediate L) in DMSO (1 mL) was added DIEA (18.7 mg, 145 umol) and KI (24.1 mg, 145 umol) and 2-[(1OS)-12-[5-(4-piperidyl)pyrimidin-2-yl]-1,5,6,8,12-pentazatricyclo[8.4.0.0^{2,7}]tetradeca-2(7),3,5-trien-4-yl]phenol (24.5 mg, 55.2 umol, Intermediate M). The mixture was stirred at 60° C. for 5 hours. On completion, the reaction was filtered to give a filtrate. The filtrate was purified by prep-HPLC (column: Phenomenex luna C18 150×25 mm×10 um; mobile phase: [water (0.225% FA)-ACN]; B %: 9%-39%,11.5 min) to give the title compound (11.6 mg, 21% yield) as a yellow gum. ¹H NMR (400 MHz, DMSO-d₆) δ=8.98 (d, J=0.8 Hz, 1H), 8.54-8.44 (m, 1H), 8.33 (d, J=5.2 Hz, 2H), 8.20 (s, 1H), 7.94 (d, J=7.6 Hz, 1H), 7.45-7.36 (m, 4H), 7.34-7.30 (m, 2H), 7.24-7.18 (m, 1H), 6.89-6.83 (m, 2H), 6.09-6.04 (m, 1H), 4.75-4.67 (m, 2H), 4.37-4.26 (m, 3H), 4.22-4.08 (m, 3H), 3.79-3.73 (m, 1H), 3.66 (d, J=9.6 Hz, 1H), 3.64-3.54 (m, 2H), 3.45 (d, J=11.2 Hz, 2H), 3.25-3.22 (m, 2H), 3.16-3.08 (m, 2H), 2.99-2.91 (m, 3H), 2.80-2.70 (m, 1H), 2.45 (s, 3H), 2.43 (d, J=3.2 Hz, 1H), 2.30-2.21 (m, 2H), 2.07-2.00 (m, 1H), 1.99-1.88 (m, 3H), 1.74-1.60 (m, 6H), 1.57-1.48 (m, 2H), 0.98-0.93 (m, 3H), 0.84-0.78 (m, 3H), LC-MS (ESI⁺) m/z 983.4 (M+H)⁺.

### Example 6: Synthesis of (2S,4R)-4-hydroxy-1-(2-(3-(4-(4-(2-((R)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)butoxy)isoxazol-5-yl)-3-methylbutanoyl)-N-(4-(4-methylthiazol-5-yl)benzyl)pyrrolidine-2-carboxamide (I-51)

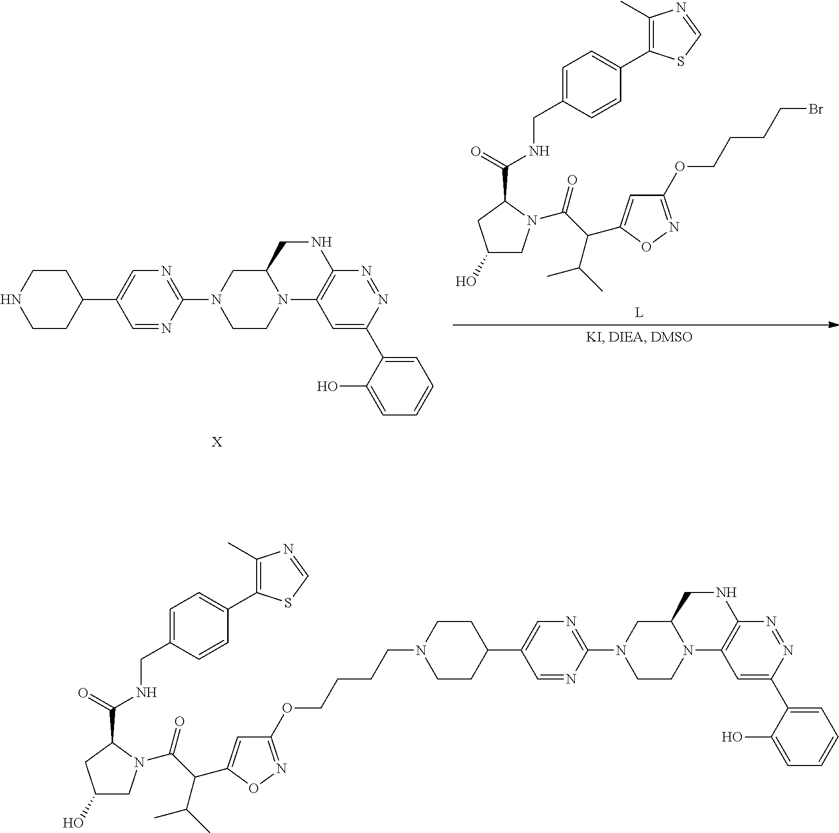

(2S,4R)-4-hydroxy-1-(2-(3-(4-(4-(2-((R)-2-(2-hydroxyphenyl)-6a,7,9,10-tetrahydro-5H-pyrazino[1′,2′:4,5]pyrazino[2,3-c]pyridazin-8(6H)-yl)pyrimidin-5-yl)piperidin-1-yl)butoxy)isoxazol-5-yl)-3-methylbutanoyl)-N-(4-(4-methylthiazol-5-yl)benzyl)pyrrolidine-2-carboxamide was synthesized via Method 3, coupling 2-[(1OR)-12-[5-(4-piperidyl)pyrimidin-2-yl]-1,5,6,8,12-pentazatricyclo[8.4.0.0^{2,7}]tetradeca-2(7),3,5-trien-4-yl]phenol (Intermediate X) with (2S,4R)-1-[2-[3-(4-bromobutoxy)isoxazol-5-yl]-3-methyl-butanoyl]-4-hydroxy-N-[[4-(4-methylthiazol-5-yl)phenyl]methyl]pyrrolidine-2-carboxamide (Intermediate L). The product was purified by prep-HPLC (column: Phenomenex Luna C18 150×25 mm×10 um; mobile phase: [water(0.225% FA)-ACN]; B %: 12%-42%, 11.5 min) to give the title compound (19.4 mg, 37% yield) as a yellow solid. ¹H NMR (400 MHz, DMSO-d₆) δ=8.98 (s, 1H), 8.49 (d, J=15.2 Hz, 1H), 8.33 (d, J=5.2 Hz, 2H), 7.94 (d, J=8.0 Hz, 1H), 7.47-7.35 (m, 4H), 7.34-7.29 (m, 2H), 7.24-7.18 (m, 1H), 6.90-6.82 (m, 2H), 6.07 (d, J=12.8 Hz, 1H), 5.12 (s, 1H), 4.72 (d, J=13.2 Hz, 2H), 4.43 (t, J=7.6 Hz, 1H), 4.38-4.27 (m, 3H), 4.23-4.08 (m, 3H), 3.79-3.73 (m, 1H), 3.68-3.54 (m, 2H), 3.48-3.40 (m, 1H), 3.28-3.23 (m, 1H), 3.17-3.10 (m, 1H), 3.03-2.91 (m, 3H), 2.80-2.71 (m, 1H), 2.52 (d, J=2.0 Hz, 1H), 2.45 (s, 3H), 2.43 (d, J=2.8 Hz, 1H), 2.40-2.36 (m, 2H), 2.30-2.18 (m, 2H), 2.09-1.97 (m, 3H), 1.94-1.86 (m, 1H), 1.75-1.63 (m, 5H), 1.62-1.49 (m, 3H), 0.95 (t, J=7.2 Hz, 3H), 0.81 (dd, J=6.8, 13.6 Hz, 2H), 0.68-0.56 (m, 1H), LC-MS (ESI⁺) m/z 983.4 (M+H)⁺.

### Example 7: Synthesis of (2S,4R)—N-(4-ethynylbenzyl)-4-hydroxy-1-((S)-2-(2-(6-(4-((S)-2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)-2-azaspiro[3.3]heptan-2-yl)acetamido)-3,3-dimethylbutanoyl)pyrrolidine-2-carboxamide (I-214)

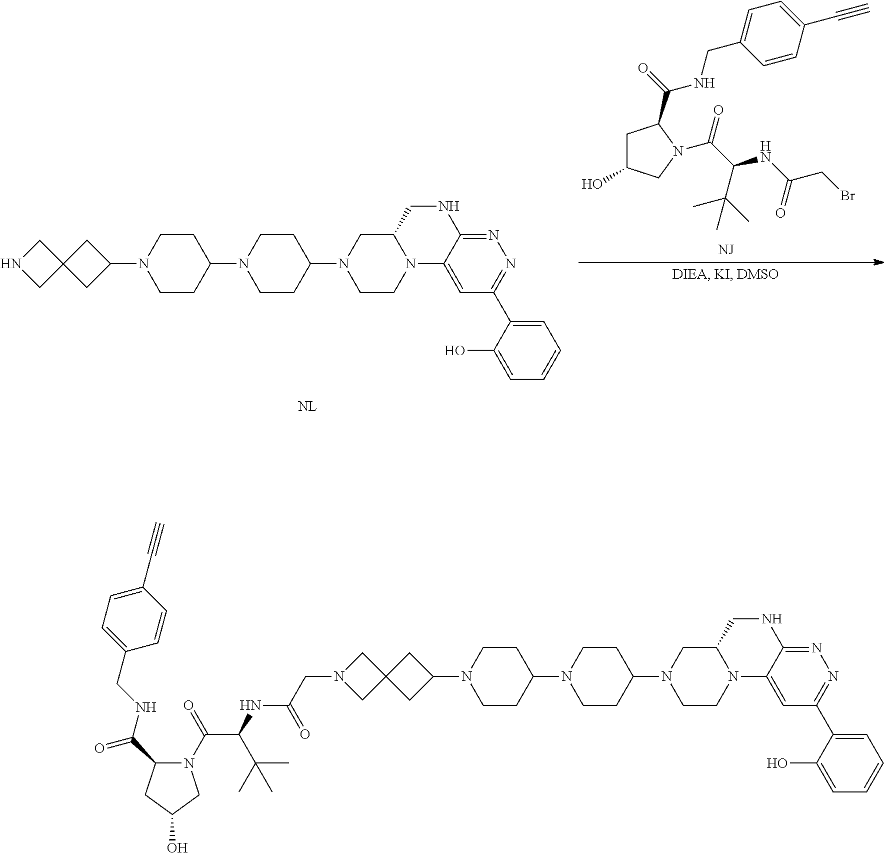 [SMILES: Oc1ccccc1-c1cc2c(nn1)NC[C@H]1CN(C3CCN(C4CCN(C5CC6(CNC6)C5)CC4)CC3)CCN21 ; C#Cc1ccc(CNC(=O)[C@@H]2C[C@@H](O)CN2C(=O)[C@@H](NC(=O)CBr)C(C)(C)C)cc1 ; "NL" ; C#Cc1ccc(CNC(=O)[C@@H]2C[C@@H](O)CN2C(=O)[C@@H](NC(=O)CN2CC3(CC(N4CCC(N5CCC(N6CCN7c8cc(-c9ccccc9O)nnc8NC[C@H]7C6)CC5)CC4)C3)C2)C(C)(C)C)cc1]

To a solution of 2-[(10S)-12-[1-[1-(2-azaspiro[3.3]heptan-6-yl)-4-piperidyl]-4-piperidyl]-1,5,6,8,12-pentazatricyclo[8.4.0.0^{2,7}]tetradeca-2,4,6-trien-4-yl]phenol (50 mg, 91.7 umol, Intermediate NL), (2S,4R)-1-[(2S)-2-[(2-bromoacetyl)amino]-3,3-dimethyl-butanoyl]-N-[(4-ethynylphenyl)methyl]-4-hydroxy-pyrrolidine-2-carboxamide (43.9 mg, 91.7 umol, Intermediate NJ) in DMSO (0.5 mL) was added DIEA (35.5 mg, 275 umol) and KI (3.05 mg, 18.3 umol). The mixture was then stirred at 50° C. for 12 hrs. On completion, the reaction mixture was filtered and concentrated under reduced pressure to give a residue. The residue was purified by prep-HPLC (neutral condition; column: Waters xbridge 150*25 mm 10 um; mobile phase: [water(NH₄HCO₃)-ACN]; B %: 26%-56%, 11 min) to give the title compound (3.42 mg, 4% yield) as an off-white solid. LC-MS (ESI+) m/z 942.6 (M+H)+. ¹H NMR (400 MHz, DMSO-d₆) δ=8.60-8.55 (m, 1H), 7.92 (br d, J=8.0 Hz, 1H), 7.55 (br d, J=10.0 Hz, 1H), 7.41-7.37 (m, 2H), 7.35-7.32 (m, 3H), 7.23-7.18 (m, 2H), 6.88-6.83 (m, 2H), 5.14 (d, J=3.6 Hz, 1H), 4.49 (d, J=9.6 Hz, 1H), 4.44-4.39 (m, 1H), 4.37-4.32 (m, 2H), 4.26-4.20 (m, 1H), 4.14-4.11 (m, 1H), 4.04 (br d, J=12.0 Hz, 1H), 3.67-3.62 (m, 1H), 3.60-3.55 (m, 1H), 3.49-3.44 (m, 1H), 3.26-3.23 (m, 2H), 3.19-3.13 (m, 5H), 3.03 (br d, J=8.4 Hz, 4H), 2.91-2.84 (m, 3H), 2.81-2.76 (m, 2H), 2.53 (br s, 2H), 2.32-2.28 (m, 1H), 2.21-2.15 (m, 3H), 2.08 (br s, 2H), 2.05-2.01 (m, 1H), 1.91 (br dd, J=3.6, 8.0 Hz, 2H), 1.87-1.82 (m, 2H), 1.78-1.73 (m, 2H), 1.68-1.60 (m, 4H), 1.40-1.31 (m, 4H), 0.93 (s, 9H).

### Example 8: Synthesis of (2S,4R)—N-(4-ethynylbenzyl)-4-hydroxy-1-((S)-2-(2-(6-(4-((S)-2-(2-hydroxyphenyl)-6a,7,9,10-tetrahydro-5H-pyrazino[1′,2′:4,5]pyrazino[2,3-c]pyridazin-8(6H)-yl)-[1,4′-bipiperidin]-1′-yl)-2-azaspiro[3.3]heptan-2-yl)acetamido)-3,3-dimethylbutanoyl)pyrrolidine-2-carboxamide (I-214)

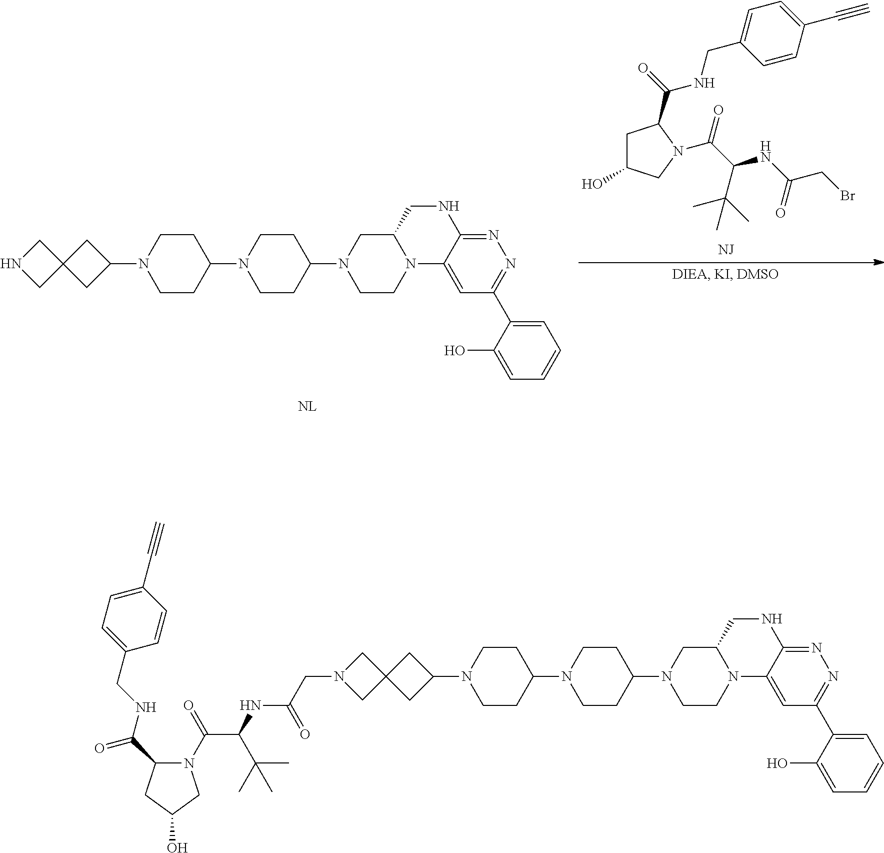 [SMILES: Oc1ccccc1-c1cc2c(nn1)NC[C@H]1CN(C3CCN(C4CCN(C5CC6(CNC6)C5)CC4)CC3)CCN21 ; C#Cc1ccc(CNC(=O)[C@@H]2C[C@@H](O)CN2C(=O)[C@@H](NC(=O)CBr)C(C)(C)C)cc1 ; "NL" ; C#Cc1ccc(CNC(=O)[C@@H]2C[C@@H](O)CN2C(=O)[C@@H](NC(=O)CN2CC3(CC(N4CCC(N5CCC(N6CCN7c8cc(-c9ccccc9O)nnc8NC[C@H]7C6)CC5)CC4)C3)C2)C(C)(C)C)cc1]

To a solution of 2-[(1OS)-12-[1-[1-(2-azaspiro[3.3]heptan-6-yl)-4-piperidyl]-4-piperidyl]-1,5,6,8,12-pentazatricyclo[8.4.0.0^{2,7}]tetradeca-2,4,6-trien-4-yl]phenol (50 mg, 91.7 umol, Intermediate NL), (2S,4R)-1-[(2S)-2-[(2-bromoacetyl)amino]-3,3-dimethyl-butanoyl]-N-[(4-ethynylphenyl)methyl]-4-hydroxy-pyrrolidine-2-carboxamide (43.9 mg, 91.7 umol, Intermediate NJ) in DMSO (0.5 mL) was added DIEA (35.5 mg, 275 umol) and KI (3.05 mg, 18.3 umol). The mixture was then stirred at 50° C. for 12 hrs. On completion, the reaction mixture was filtered and concentrated under reduced pressure to give a residue. The residue was purified by prep-HPLC (neutral condition; column: Waters xbridge 150*25 mm 10 um; mobile phase: [water(NH₄HCO₃)-ACN]; B %: 26%-56%, 11 min) to give the title compound (3.42 mg, 4% yield) as an off-white solid. LC-MS (ESI+) m/z 942.6 (M+H)+. ¹H NMR (400 MHz, DMSO-d₆) δ=8.60-8.55 (m, 1H), 7.92 (br d, J=8.0 Hz, 1H), 7.55 (br d, J=10.0 Hz, 1H), 7.41-7.37 (m, 2H), 7.35-7.32 (m, 3H), 7.23-7.18 (m, 2H), 6.88-6.83 (m, 2H), 5.14 (d, J=3.6 Hz, 1H), 4.49 (d, J=9.6 Hz, 1H), 4.44-4.39 (m, 1H), 4.37-4.32 (m, 2H), 4.26-4.20 (m, 1H), 4.14-4.11 (m, 1H), 4.04 (br d, J=12.0 Hz, 1H), 3.67-3.62 (m, 1H), 3.60-3.55 (m, 1H), 3.49-3.44 (m, 1H), 3.26-3.23 (m, 2H), 3.19-3.13 (m, 5H), 3.03 (br d, J=8.4 Hz, 4H), 2.91-2.84 (m, 3H), 2.81-2.76 (m, 2H), 2.53 (br s, 2H), 2.32-2.28 (m, 1H), 2.21-2.15 (m, 3H), 2.08 (br s, 2H), 2.05-2.01 (m, 1H), 1.91 (br dd, J=3.6, 8.0 Hz, 2H), 1.87-1.82 (m, 2H), 1.78-1.73 (m, 2H), 1.68-1.60 (m, 4H), 1.40-1.31 (m, 4H), 0.93 (s, 9H).

### Example 9: Synthesis of (2S,4R)—N-(4-ethynylbenzyl)-4-hydroxy-1-((S)-2-(2-(4-(6-((S)-2-(2-hydroxyphenyl)-6a,7,9,10-tetrahydro-5H-pyrazino[1′,2′:4,5]pyrazino[2,3-c]pyridazin-8(6H)-yl)-2-azaspiro[3.3]heptan-2-yl)piperidin-1-yl)acetamido)-3,3-dimethylbutanoyl)pyrrolidine-2-carboxamide (I-252)

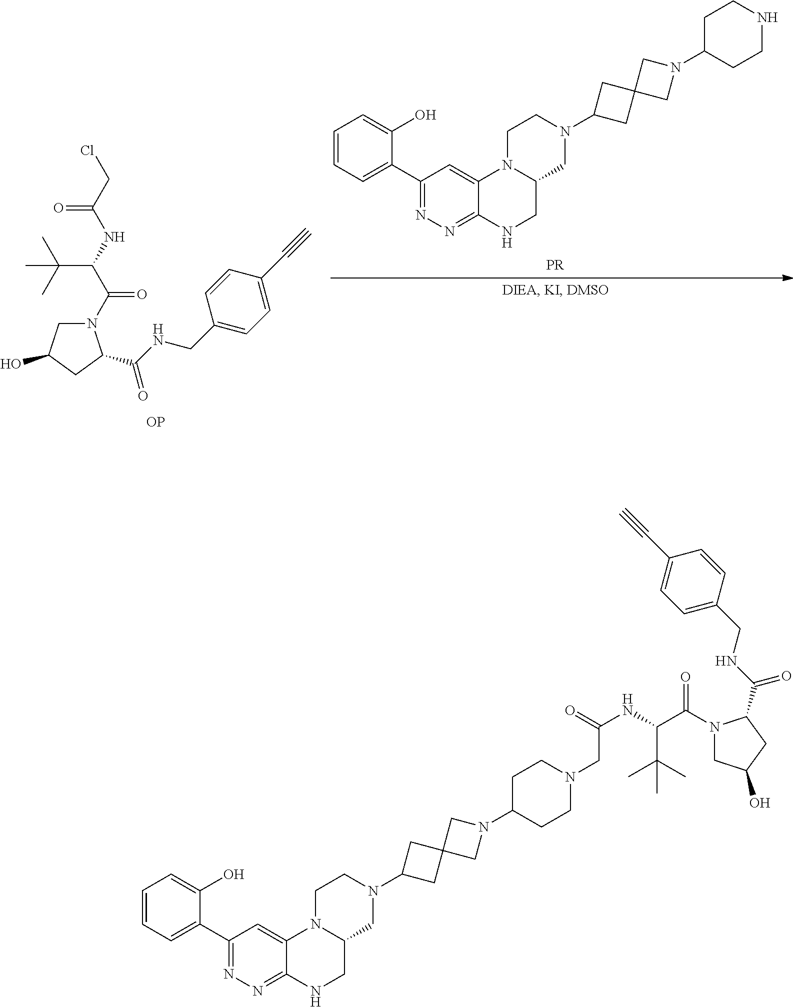

To a mixture of (2S,4R)-1-((S)-2-(2-chloroacetamido)-3,3-dimethylbutanoyl)-N-(4-ethynylbenzyl)-4-hydroxypyrrolidine-2-carboxamide (41.5 mg, 95.6 umol, Intermediate OP) and (S)-2-(8-(2-(piperidin-4-yl)-2-azaspiro[3.3]heptan-6-yl)-6,6a,7,8,9,10-hexahydro-5H-pyrazino[1′,2′:4,5]pyrazino[2,3-c]pyridazin-2-yl)phenol (50 mg, 86.9 umol, Intermediate PR) in DMSO (1 mL) was added DIEA (33.7 mg, 261 umol) and KI (2.88 mg, 17.4 umol). The mixture was stirred at 80° C. for 1 hr. On completion, the reaction mixture was filtered and concentrated under reduced pressure to give a residue. The residue was purified by prep-HPLC (FA condition column: Waters xbridge 150*25 mm 10 um;mobile phase: [water(NH4HCO3)-ACN];B %: 25%-55%, min) to give the title compound as a white solid (21.1 mg, 27% yield). LC-MS (ESI+) m/z 859.6 (M+H)⁺. ¹H NMR (400 MHz, DMSO-d₆) δ=8.61-8.54 (m, 1H), 7.91 (d, J=7.2 Hz, 1H), 7.83-7.73 (m, 1H), 7.42-7.37 (m, 2H), 7.35-7.30 (m, 3H), 7.23-7.17 (m, 2H), 6.88-6.83 (m, 2H), 5.13 (s, 1H), 4.47 (d, J=9.6 Hz, 1H), 4.41 (t, J=8.4 Hz, 1H), 4.37-4.30 (m, 2H), 4.27 (d, J=5.2 Hz, 1H), 4.23 (d, J=5.6 Hz, 1H), 4.13 (s, 1H), 4.05 (d, J=12 Hz, 1H), 3.68-3.63 (m, 1H), 3.62-3.56 (m, 1H), 3.51-3.46 (m, 1H), 3.17 (t, J=9.6 Hz, 2H), 3.13-3.09 (m, 2H), 3.03-2.96 (m, 3H), 2.93-2.86 (m, 3H), 2.75-2.67 (m, 2H), 2.64-2.59 (m, 1H), 2.21-2.08 (m, 4H), 2.07-2.00 (m, 1H), 1.95-1.84 (m, 5H), 1.64-1.53 (m, 3H), 1.25-1.13 (m, 2H), 0.93 (s, 9H).

### Example 10. MSD SMARCA2 Degradation in A549 Cell Line

Cells were seeded into 96-well plates (A549 cells: 2×104 cells/well/100 ul media) and incubated overnight. The next day, 200 nL compound were added into the intermediate plate with Echo (Labcyte 550) from source plate containing a 3-fold serial dilution from top concentration of 1 mM. The culture medium was changed with 80 ul of fresh medium and 80 uL of 2×compound solution was added into the well to make a final concentration of 1000 nM, 333.3 nM, 111.1 nM, 37.04 nM, 12.35 nM, 4.115 nM, 1.372 nM, 0.457 nM, 0.152 nM and 0 nM (DMSO). The wells were mixed and then incubated for 24 hours. The media was aspirated from the cultures and 60 ul pre-chilled PIPA lysis buffer (Boston BioProducts BP-115D) with protease/phosphatase inhibitor (Roche 05892791001/Roche 04906837001) was added into the well to lyze the cells for 20 minutes at 4° C. The MSD plate (L15XA) was coated with 40 uL cell lysate and incubated at 4° C. overnight. The next day, the plate was washed three times with TBST (CST #9997S), 150 ul/well. The MSD plates was blocked with 150 ul blocking buffer per well and shaked for 1 hr at RT, 600 rpm. The blocking buffer was 3% Blocker A (MSD, R93BA-4) in TBST. The MSD plate was washed three times with 150 uL/well of TBST and 25 ul/well of detection antibody (Rabbit anti-SMARCA2/BRM antibody, 100 μg/mL, ab223735) was added at final concentration of 1 ug/mL diluted in 1% Blocking buffer and shaken for 1 hour at RT, 600 rpm. The MSD plate was washed three times with 150 ul/well of TBST and 25 ul/well of SULFO-TAG anti-rabbit antibody (MSD, R32AB-1) was added at final concentration of 1 ug/ml diluted in 1% Blocking buffer and shaken for 1 hour at RT, 600 rpm. The MSD plate was washed three times with 150 uL/well of TBST and 150 ul/well of 2×MSD reading buffer diluted from 4×(MSD, R92TC-2) with water was added. Lastly, the MSD instrument was read.

SMARCA2 protein degradation in A549 cells for compounds of the invention are presented in Table 7. The letter codes for SMARCA2 degradation potency (DC₅₀) include: A (<10 nM), B (10-50 nM), C (>50-100 nM), and D (>100 nM). The letter codes for the percentage of SMARCA degradation after 24 hours (Dmax %) include: A (>90% degradation), B (>70-90% degradation), C (50-70% degradation), and D (<50% degradation).

TABLE 7
SMARCA2 Degradation Results

| | SMARCA2 | |
|---|---|---|
| | MSD A549 | SMARCA2 |
| | degradation | MSD A549 |
| | 24 h: Average | degradation |
| | external-Abs | 24 h: Average |
| I-# | DC50 (nM) | Dmax % |
| I-1 | A | A |
| I-2 | B | B |
| I-3 | D | B |
| I-4 | D | C |
| I-5 | B | A |
| I-6 | D | A |
| I-7 | B | A |
| I-8 | B | A |
| I-9 | B | B |
| I-10 | B | A |
| I-11 | B | A |
| I-12 | C | B |
| I-13 | B | A |
| I-14 | A | B |
| I-15 | B | A |
| I-16 | C | B |
| I-18 | — | D |
| I-19 | B | A |
| I-21 | B | A |
| I-22 | B | A |
| I-27 | B | B |
| I-28 | C | B |
| I-29 | D | C |
| I-30 | A | A |
| I-31 | C | A |
| I-32 | A | A |
| I-33 | D | C |
| I-34 | D | B |
| I-35 | B | B |
| I-36 | B | A |
| I-37 | A | A |
| I-38 | B | A |
| I-39 | D | C |
| I-40 | D | B |
| I-41 | C | B |
| I-47 | D | A |
| I-48 | D | B |
| I-49 | B | A |
| I-50 | A | A |
| I-51 | B | B |
| I-52 | B | A |
| I-53 | B | A |
| I-58 | B | A |
| I-59 | — | D |
| I-60 | D | B |
| I-61 | D | C |
| I-62 | D | B |
| I-63 | B | A |
| I-64 | B | A |
| I-73 | A | A |
| I-74 | B | A |
| I-75 | D | A |
| I-76 | B | A |
| I-77 | B | A |
| I-78 | B | A |
| I-79 | D | A |
| I-80 | A | A |
| I-81 | B | A |
| I-82 | A | A |
| I-83 | C | B |
| I-85 | C | A |
| I-86 | A | A |
| I-87 | B | A |
| I-88 | A | A |
| I-89 | D | A |
| I-90 | D | B |
| I-91 | D | A |
| I-92 | D | A |
| I-93 | D | A |
| I-94 | B | A |
| I-95 | D | D |
| I-96 | A | A |
| I-97 | B | A |
| I-98 | B | A |
| I-99 | A | A |
| I-100 | A | A |
| I-101 | D | C |
| I-102 | B | A |
| I-103 | C | A |
| I-104 | D | A |
| I-105 | D | A |
| I-106 | D | D |
| I-111 | A | A |
| I-112 | C | A |
| I-113 | B | A |
| I-114 | D | D |
| I-115 | D | D |
| I-116 | B | A |
| I-117 | D | A |
| I-118 | D | B |
| I-119 | A | A |
| I-120 | B | A |
| I-121 | C | A |
| I-122 | B | A |
| I-125 | A | A |
| I-126 | D | D |
| I-127 | D | A |
| I-128 | A | A |
| I-129 | B | A |
| I-130 | A | A |
| I-131 | B | A |
| I-132 | D | D |
| I-133 | D | B |
| I-136 | B | A |
| I-137 | C | A |
| I-138 | A | A |
| I-139 | D | D |
| I-140 | D | D |
| I-141 | D | D |
| I-143 | A | A |
| I-144 | B | A |
| I-145 | B | A |
| I-146 | A | A |
| I-147 | C | A |
| I-148 | C | A |
| I-149 | B | A |
| I-150 | B | A |
| I-151 | B | A |
| I-152 | D | B |
| I-153 | D | A |
| I-154 | D | A |
| I-155 | B | A |
| I-156 | B | A |
| I-157 | C | A |
| I-158 | C | A |
| I-159 | D | A |
| I-160 | D | A |
| I-161 | D | A |
| I-162 | D | A |
| I-163 | B | A |
| I-164 | D | A |
| I-165 | D | A |
| I-166 | A | A |
| I-167 | B | A |
| I-168 | A | A |
| I-169 | B | A |
| I-170 | B | A |
| I-171 | D | A |
| I-172 | D | A |
| I-173 | D | A |
| I-174 | D | A |
| I-175 | C | A |
| I-176 | D | A |
| I-177 | D | A |
| I-178 | D | A |
| I-179 | D | A |
| I-180 | D | A |
| I-181 | D | A |
| I-182 | D | A |
| I-183 | D | A |
| I-184 | D | A |
| I-185 | D | A |
| I-186 | C | A |
| I-187 | C | A |
| I-188 | D | A |
| I-189 | D | C |
| I-190 | D | A |
| I-191 | D | A |
| I-192 | D | B |
| I-193 | D | B |
| I-194 | D | B |
| I-195 | B | A |
| I-196 | B | A |
| I-197 | D | B |
| I-198 | D | B |
| I-199 | D | A |
| I-200 | D | A |
| I-201 | D | A |
| I-202 | D | A |
| I-203 | D | B |
| I-204 | D | B |
| I-205 | D | A |
| I-206 | D | B |
| I-207 | D | A |
| I-208 | D | B |
| I-209 | D | B |
| I-210 | D | A |
| I-211 | C | A |
| I-212 | D | A |
| I-213 | D | A |
| I-214 | D | A |
| I-215 | C | A |
| I-216 | D | B |
| I-217 | D | B |
| I-218 | D | C |
| I-219 | D | C |
| I-220 | D | A |
| I-221 | D | A |
| I-222 | D | A |
| I-223 | C | A |
| I-224 | C | A |
| I-225 | D | A |
| I-226 | D | A |
| I-227 | C | A |
| I-228 | D | A |
| I-229 | D | A |
| I-230 | B | A |
| I-231 | C | A |
| I-232 | B | A |
| I-233 | B | A |
| I-234 | C | A |
| I-235 | D | C |
| I-236 | D | B |
| I-237 | C | A |
| I-238 | D | A |
| I-239 | D | A |
| I-240 | D | A |
| I-241 | C | A |
| I-242 | C | A |
| I-243 | C | A |
| I-244 | D | A |
| I-245 | D | B |
| I-246 | D | A |
| I-247 | C | A |
| I-248 | D | D |
| I-249 | B | A |
| I-250 | B | A |
| I-251 | D | A |
| I-252 | C | A |
| I-253 | D | D |
| I-254 | C | B |
| I-255 | D | B |
| I-256 | D | D |
| I-258 | D | B |
| I-259 | A | A |
| I-262 | D | C |
| I-263 | D | D |
| I-266 | C | A |
| I-267 | C | B |
| I-268 | D | A |
| I-269 | B | A |
| I-270 | A | A |
| I-271 | C | A |
| I-272 | D | C |
| I-273 | D | A |
| I-274 | D | A |
| I-275 | D | A |
| I-276 | A | A |
| I-277 | A | A |
| I-281 | D | D |
| I-282 | A | A |
| I-283 | D | B |
| I-284 | B | A |
| I-285 | B | A |
| I-286 | D | C |
| I-287 | A | A |
| I-288 | A | A |
| I-289 | B | A |
| I-290 | B | A |
| I-291 | B | A |
| I-292 | A | A |
| I-293 | B | A |
| I-294 | A | A |
| I-296 | C | A |
| I-298 | B | B |
| I-299 | A | A |
| I-300 | D | A |
| I-301 | B | A |
| I-302 | B | A |
| I-303 | D | A |
| I-304 | C | A |
| I-305 | D | C |
| I-306 | D | D |
| I-307 | D | D |
| I-309 | B | A |
| I-310 | B | A |
| I-311 | B | A |
| I-312 | B | A |
| I-316 | D | D |
| I-317 | B | A |
| I-318 | B | A |
| I-319 | B | A |
| I-320 | A | A |
| I-321 | B | A |
| I-323 | B | A |
| I-324 | D | D |
| I-325 | B | A |
| I-327 | C | A |
| I-328 | D | D |
| I-329 | D | D |
| I-330 | D | C |
| I-331 | D | C |
| I-332 | A | A |
| I-333 | B | A |
| I-334 | D | C |
| I-335 | D | B |
| I-337 | D | A |
| I-338 | D | D |
| I-339 | D | D |
| I-340 | D | C |
| I-341 | D | D |
| I-342 | D | D |
| I-343 | D | D |
| I-344 | D | D |
| I-362 | B | A |
| I-363 | B | A |
| I-364 | D | B |
| I-365 | D | A |
| I-368 | D | D |
| I-369 | A | A |
| I-402 | B | A |
| I-403 | B | A |
| I-410 | B | A |
| I-416 | B | A |

### Example 11. SMARCA2 and SMARCA4 Western Blot MV4-11 Degradation

Cells were seeded into 6-well plates (MV4-11: 4×106 cells/well/1 ml) and 1 ml of 2×compound solution was added into the well to make the final concentration and the plates were mixed well and incubated for 24 hours (No cytotoxicity was observed). The cell were collected with media and spun at 3000 rpm for 5 minutes. The supernatant was aspirated and the well and the cells were washed with cold PBS once and combined for centrifugation again; the supernatant aspirated again. 200 uL pre-chilled RIPA lysis buffer (Boston BioProducts BP-115D) with protease/phosphatase inhibitor (Roche 05892791001/Roche 04906837001) was directly added into the tube to lyze the cells for 20 minutes on ice. The cell lysate were collected into EP tubes and spun at 13000 rpm for 20 minutes and 72 uL supernatant was transferred to a fresh EP tube containing 18 uL of 5×loading buffer (Beyotime Bio P0015) to make the loading samples. The samples were heated to 100° C. for 10 minutes and cooled to RT and microcentrifuged. 20 uL of samples were loaded onto SDS-PAGE gel (Novex, WG1402BOX) and the gel was run at 80 V for 20 minutes and 120 V for 1.5 hours. The samples were electrotransfer to a NC membrane using wet-transfer method with 250 mA for 2.5 hours. The membrane was blocked with LICOR blocking buffer (LI COR,927-50000) for 1 hour. The membrane was washed three times with TBST(CST #9997S), 5 minutes each. Incubation was performed with primary antibody prepared in blocking buffer with 0.1% Tween-20 (Solarbio, P8220) at 4° C. overnight (Anti-SMARCA2/BRM antibody (abl5597) 1:500; Anti-BRG1 antibody [EPR3912] (ab108318) 1:1000; Rabbit anti-Baf180 antibody [EPR15860] (Abcam, ab196022) 1:1000; mouse anti-beta-Actin (8H10D10) (CST #3700) 1:10000). The membrane was washed three times with TBST, 5 minutes each. Incubation with secondary antibody was performed for 1 hour at RT (anti-rabbit IgG (Licor,926-32211) 1:5000; anti-mouse IgG (LI-COR, 926-68070) 1:5000). The membrane was washed three times with 5 minutes each and lastly the LiCOR was read.

SMARCA2/SMARCA4 protein degradation selectivity in MV4-11 cells for compounds of the invention are presented in Table 8. The letter codes for SMARCA2/4 degradation potency (DC₅₀) include: A (<1 nM), B (1-10 nM), C (>10-100 nM), and D (>100 nM). The letter codes for the percentage of SMARCA2/4 degradation after 24 hours (Dmax %) include: A (>90% degradation), B (>70-90% degradation), C (50-70% degradation), and D (<50% degradation). The letter codes of SMARCA2/4 selectivity include: A (>500 fold), B (>100-500 fold), C (10-100 fold), and D (<10 fold). Table 8. SMARCA2/4 Selectivity Results

TABLE 8
SMARCA2/4 Selectivity Results

| | SMARCA2 | | SMARCA4 | | |
|---|---|---|---|---|---|
| | MSD MV411 | SMARCA2 | MSD MV411 | SMARCA4 | |
| | degradation | MSD MV411 | degradation | MSD MV411 | |
| | 24 h: Mean | degradation | 24 h: Mean | degradation | |
| | external-Abs | 24 h: average | external-Abs | 24 h: average | Selectivity |
| I-# | DC50 (nM) | Dmax % | DC50 (nM) | Dmax % | in MV411 |
| I-1 | B | A | B | A | D |
| I-2 | B | A | C | A | D |
| I-3 | B | B | D | C | A |
| I-4 | B | B | D | D | B |
| I-5 | B | A | D | D | A |
| I-7 | B | A | C | A | C |
| I-8 | B | B | C | B | D |
| I-9 | B | A | C | B | C |
| I-10 | A | A | B | A | D |
| I-11 | B | A | C | A | D |
| I-12 | C | B | C | A | D |
| I-13 | B | A | C | A | C |
| I-14 | A | A | B | A | D |
| I-15 | B | A | C | A | C |
| I-16 | B | A | C | B | D |
| I-17 | D | D | D | D | C |
| I-18 | C | D | C | D | C |
| I-19 | B | A | C | B | C |
| I-20 | D | D | D | D | C |
| I-21 | A | A | B | A | C |
| I-22 | A | A | C | B | C |
| I-23 | C | B | D | D | C |
| I-24 | A | A | B | A | D |
| I-25 | A | B | C | B | C |
| I-26 | B | B | D | C | B |
| I-27 | B | A | D | D | A |
| I-28 | C | C | D | D | C |
| I-29 | C | C | D | D | B |
| I-30 | A | A | A | A | D |
| I-31 | C | B | D | C | B |
| I-32 | A | A | B | A | C |
| I-33 | C | C | D | D | C |
| I-34 | C | D | D | C | B |
| I-35 | B | A | B | B | D |
| I-36 | B | A | B | A | D |
| I-37 | A | A | A | A | D |
| I-38 | A | A | C | A | C |
| I-40 | C | B | D | B | D |
| I-41 | A | A | C | A | D |
| I-42 | C | C | D | B | C |
| I-43 | D | D | D | D | C |
| I-44 | C | B | D | B | D |
| I-45 | A | A | B | A | D |
| I-46 | A | A | B | A | D |
| I-47 | B | A | B | A | D |
| I-48 | C | C | D | A | C |
| I-49 | A | A | B | A | C |
| I-50 | A | A | B | A | D |
| I-51 | A | A | B | A | D |
| I-52 | A | A | B | A | D |
| I-53 | A | A | B | A | D |
| I-54 | B | A | C | B | C |
| I-55 | A | A | A | A | D |
| I-56 | B | B | D | C | A |
| I-57 | B | B | D | D | B |
| I-58 | B | B | D | C | B |
| I-62 | D | D | D | D | C |
| I-63 | C | B | D | C | C |
| I-64 | B | A | C | C | C |
| I-65 | B | A | C | B | C |
| I-66 | C | B | D | D | B |
| I-67 | D | D | D | D | C |
| I-68 | C | C | D | D | C |
| I-69 | A | A | B | A | D |
| I-70 | B | B | D | D | A |
| I-71 | B | B | D | D | A |
| I-72 | A | B | B | B | D |
| I-73 | B | A | C | B | C |
| I-74 | A | A | B | A | C |
| I-75 | B | B | D | D | A |
| I-76 | B | B | D | C | C |
| I-77 | B | B | D | D | C |
| I-78 | C | B | D | D | B |
| I-79 | C | B | D | D | C |
| I-80 | A | A | B | A | C |
| I-81 | B | A | C | B | C |
| I-82 | B | A | C | B | C |
| I-83 | B | A | C | A | C |
| I-85 | C | B | D | D | C |
| I-86 | A | A | B | A | C |
| I-87 | C | B | C | A | C |
| I-88 | A | A | B | A | C |
| I-89 | B | B | D | D | A |
| I-90 | C | B | D | D | C |
| I-91 | B | B | D | D | A |
| I-92 | B | B | D | C | C |
| I-93 | C | B | D | C | C |
| I-94 | B | B | C | C | C |
| I-95 | D | D | D | D | C |
| I-96 | A | A | B | A | C |
| I-97 | B | B | D | D | A |
| I-98 | B | B | D | D | A |
| I-99 | B | B | D | C | B |
| I-100 | A | A | C | B | C |
| I-101 | D | D | D | D | C |
| I-102 | B | B | D | C | C |
| I-103 | B | C | D | B | C |
| I-104 | D | D | D | B | C |
| I-105 | C | C | — | — | — |
| I-106 | D | D | — | — | — |
| I-111 | A | A | C | A | C |
| I-112 | C | C | — | — | — |
| I-113 | B | B | D | C | B |
| I-114 | D | D | D | D | C |
| I-115 | D | D | — | — | — |
| I-116 | B | B | D | D | B |
| I-117 | C | C | D | D | C |
| I-118 | C | C | — | — | — |
| I-119 | B | A | C | A | C |
| I-120 | C | B | D | C | B |
| I-121 | C | C | D | D | B |
| I-122 | B | B | C | C | C |
| I-125 | B | B | D | C | C |
| I-126 | D | D | D | D | C |
| I-127 | C | B | D | B | D |
| I-128 | B | A | C | A | D |
| I-129 | B | A | C | A | C |
| I-130 | B | A | C | A | D |
| I-131 | A | A | C | C | C |
| I-132 | D | D | — | — | — |
| I-133 | C | B | D | C | C |
| I-136 | B | B | D | B | C |
| I-137 | B | B | D | D | A |
| I-138 | B | A | C | A | C |
| I-139 | D | D | D | C | C |
| I-140 | D | D | D | D | C |
| I-141 | D | D | D | D | C |
| I-143 | B | A | C | A | C |
| I-144 | B | B | D | B | C |
| I-145 | A | A | C | A | C |
| I-146 | B | A | C | B | C |
| I-147 | B | A | C | B | D |
| I-148 | C | B | D | D | B |
| I-149 | B | A | C | A | D |
| I-150 | B | B | D | B | C |
| I-151 | B | B | D | C | C |
| I-152 | C | C | — | — | — |
| I-153 | B | B | — | — | — |
| I-154 | C | C | — | — | — |
| I-155 | B | B | D | D | A |
| I-156 | B | B | C | B | C |
| I-157 | B | B | — | — | — |
| I-158 | B | B | C | B | C |
| I-159 | C | C | D | C | B |
| I-160 | C | B | D | A | D |
| I-161 | C | C | D | C | C |
| I-162 | C | C | D | C | C |
| I-163 | A | B | D | C | B |
| I-164 | C | B | D | D | B |
| I-165 | C | B | D | B | D |
| I-166 | B | A | C | A | C |
| I-167 | B | B | D | B | C |
| I-168 | A | A | A | A | C |
| I-169 | B | B | D | D | B |
| I-170 | B | B | D | C | C |
| I-171 | C | C | D | D | C |
| I-172 | C | C | D | B | C |
| I-173 | C | C | D | C | C |
| I-174 | C | B | D | D | B |
| I-175 | B | B | D | D | B |
| I-176 | C | C | D | D | C |
| I-177 | C | B | D | C | C |
| I-178 | C | B | D | C | B |
| I-179 | C | B | D | C | B |
| I-180 | C | B | D | A | D |
| I-181 | C | C | D | D | B |
| I-182 | C | C | D | B | D |
| I-183 | C | B | D | B | C |
| I-184 | D | D | D | D | C |
| I-185 | C | B | D | B | C |
| I-186 | C | B | C | A | D |
| I-187 | B | B | D | B | C |
| I-188 | C | C | D | D | C |
| I-189 | D | D | D | D | C |
| I-190 | C | C | D | C | C |
| I-191 | B | B | D | B | C |
| I-192 | D | D | D | D | C |
| I-193 | D | D | D | D | C |
| I-194 | D | D | D | D | C |
| I-195 | B | A | C | B | C |
| I-196 | B | B | D | C | C |
| I-197 | D | D | D | D | C |
| I-198 | C | C | D | D | C |
| I-199 | C | C | D | D | C |
| I-200 | C | C | D | D | C |
| I-201 | B | B | D | C | C |
| I-202 | B | B | D | C | C |
| I-203 | D | D | D | D | C |
| I-204 | D | D | D | D | C |
| I-205 | D | D | D | D | C |
| I-206 | D | D | D | D | C |
| I-207 | B | B | D | D | B |
| I-208 | D | D | D | D | C |
| I-209 | D | D | D | D | C |
| I-210 | C | C | D | D | C |
| I-211 | B | B | D | D | A |
| I-212 | C | B | D | D | C |
| I-213 | B | B | D | D | B |
| I-214 | C | B | D | D | B |
| I-215 | B | A | C | B | C |
| I-216 | D | D | D | D | C |
| I-217 | C | C | D | D | C |
| I-218 | D | D | D | D | C |
| I-219 | D | D | D | D | C |
| I-220 | C | B | D | D | B |
| I-221 | C | C | D | D | C |
| I-222 | C | B | D | D | B |
| I-223 | B | B | D | C | C |
| I-224 | B | B | C | B | C |
| I-225 | B | B | D | D | B |
| I-226 | C | B | D | D | B |
| I-227 | C | B | D | A | D |
| I-228 | C | B | D | A | D |
| I-229 | C | B | D | C | B |
| I-230 | B | B | D | C | B |
| I-231 | B | A | D | C | C |
| I-232 | B | A | C | B | C |
| I-233 | B | B | D | C | C |
| I-234 | C | B | D | C | C |
| I-235 | D | D | D | D | D |
| I-236 | D | C | D | D | D |
| I-237 | B | B | D | D | B |
| I-238 | C | B | D | D | C |
| I-239 | B | A | C | A | D |
| I-240 | B | A | C | A | D |
| I-241 | B | A | C | B | C |
| I-242 | B | A | C | B | C |
| I-243 | B | B | D | D | B |
| I-244 | C | A | D | C | C |
| I-245 | C | C | D | D | B |
| I-246 | C | B | D | C | C |
| I-247 | B | B | D | C | C |
| I-248 | D | D | D | D | D |
| I-249 | C | A | D | B | C |
| I-250 | C | A | D | B | C |
| I-251 | C | B | D | D | B |
| I-252 | A | A | B | A | D |
| I-253 | D | D | D | D | D |
| I-254 | C | C | D | D | B |
| I-255 | C | B | D | D | B |
| I-256 | D | D | D | D | D |
| I-258 | C | C | D | D | C |
| I-259 | B | B | D | D | A |
| I-262 | C | B | D | C | C |
| I-263 | D | D | D | D | C |
| I-266 | C | A | D | C | C |
| I-267 | C | B | D | D | B |
| I-268 | C | B | D | C | C |
| I-269 | C | B | D | C | C |
| I-270 | B | A | D | A | C |
| I-271 | C | B | D | D | C |
| I-272 | D | C | D | D | C |
| I-273 | B | A | D | C | C |
| I-274 | B | A | C | A | C |
| I-275 | B | A | C | A | C |
| I-276 | B | A | C | A | C |
| I-277 | B | A | C | B | C |
| I-281 | D | D | D | D | D |
| I-282 | B | B | D | B | C |
| I-283 | D | D | D | D | D |
| I-284 | B | A | D | C | C |
| I-285 | C | B | D | B | C |
| I-286 | D | D | D | D | D |
| I-287 | B | B | D | C | A |
| I-288 | B | A | C | B | C |
| I-289 | C | B | D | D | B |
| I-290 | C | B | D | C | B |
| I-291 | B | A | C | A | C |
| I-292 | B | A | C | A | C |
| I-293 | B | B | D | C | B |
| I-294 | B | A | C | B | C |
| I-296 | B | A | C | B | C |
| I-298 | D | D | D | D | D |
| I-299 | B | B | D | C | C |
| I-300 | C | B | D | D | C |
| I-301 | B | A | C | A | C |
| I-302 | B | A | C | A | C |
| I-303 | C | B | D | D | B |
| I-304 | B | B | D | D | B |
| I-305 | D | C | D | D | D |
| I-306 | D | D | D | D | D |
| I-307 | D | D | D | D | D |
| I-309 | C | A | D | B | C |
| I-310 | B | A | D | A | C |
| I-311 | B | B | D | B | C |
| I-312 | C | B | D | D | B |
| I-316 | D | D | D | D | C |
| I-317 | B | B | D | B | C |
| I-318 | B | B | D | B | C |
| I-319 | B | B | D | C | B |
| I-320 | B | A | C | A | C |
| I-321 | B | B | D | D | A |
| I-323 | B | B | D | C | C |
| I-324 | D | D | D | D | C |
| I-325 | B | A | C | A | C |
| I-327 | C | B | D | B | C |
| I-328 | D | D | D | D | D |
| I-329 | D | D | D | D | D |
| I-330 | D | C | D | D | C |
| I-331 | D | C | D | D | C |
| I-332 | A | A | B | A | C |
| I-333 | A | A | C | B | C |
| I-334 | D | D | D | D | D |
| I-335 | C | C | D | B | D |
| I-337 | C | B | D | D | B |
| I-338 | D | D | D | D | C |
| I-339 | D | D | D | D | C |
| I-340 | D | D | D | D | C |
| I-341 | D | D | D | D | C |
| I-342 | D | D | D | D | C |
| I-343 | D | D | D | D | C |
| I-344 | D | D | D | D | C |
| I-362 | B | B | D | B | C |
| I-363 | B | B | D | B | C |
| I-364 | C | C | D | D | B |
| I-365 | B | B | D | D | A |
| I-368 | D | D | D | D | C |
| I-369 | B | A | D | B | C |
| I-402 | B | B | D | A | C |
| I-403 | C | B | D | B | C |
| I-410 | B | B | D | B | C |
| I-416 | B | A | C | A | C |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I-a: 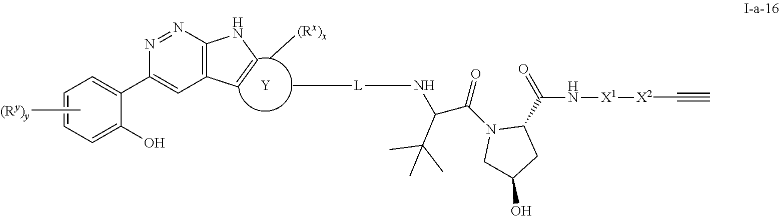
or a pharmaceutically acceptable salt thereof, wherein:
Ring Y is a fused ring selected from 6-membered aryl, 5-6 membered heteroaryl containing 1-4 heteroatoms independently selected from nitrogen, oxygen, and sulfur, and a 4-9 membered saturated or partially unsaturated monocyclic, bicyclic, or bridged bicyclic carbocyclyl or heterocyclyl with 1-4 heteroatoms independently selected from, nitrogen, oxygen, and sulfur;
each of R^{x} and R^{y} is independently hydrogen, R^{z}, halogen, —CN, —NO₂, —OR, —SR, —N(R)₂, —Si(R)₃, —S(O)₂R, —S(O)₂N(R)₂, —S(O)R, —CF(R)₂, —CF₂R, —CF₃, —C(O)R, —C(O)OR, —C(O)N(R)₂, —C(O)NROR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)R₂, —NRP(O)(OR)₂, —NRP(O)(OR)N(R)₂, —NRP(O)(N(R)₂)₂, or —NRS(O)₂R; or
two RX groups or two R^{y} groups are optionally taken together to form an optionally substituted 5-7 membered partially unsaturated or aryl fused ring having 0-2 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form an optionally substituted 3-7 membered saturated or partially unsaturated carbocyclic ring or an optionally substituted 3-7 membered saturated, partially unsaturated, or heteroaryl ring having 0-3 heteroatoms, in addition to the atom to which they are attached, independently selected from nitrogen, oxygen, and sulfur;
each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
x is 0, 1, 2, 3, 4, or 5;
y is 0, 1, 2, 3, 4, or 5;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 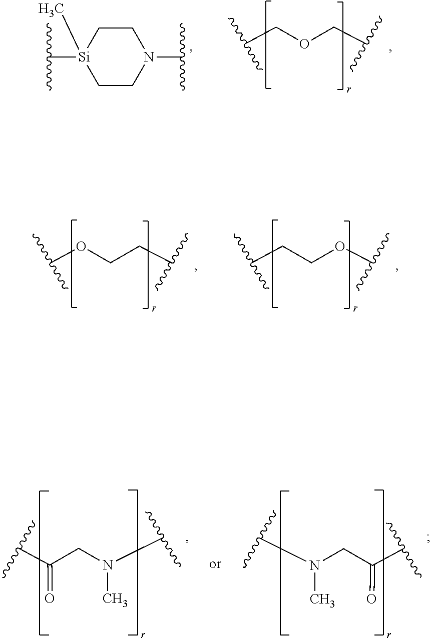
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X¹ is a covalent bond or bivalent group selected from —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
n is 0, 1, 2, 4, or 5.

2-4. (canceled)

5. The compound of claim 1, wherein Ring Y is a 4-9 membered saturated or partially unsaturated monocyclic, bicyclic, or bridged bicyclic heterocyclyl with 1-4 heteroatoms independently selected from, nitrogen, oxygen, and sulfur.

6. The compound of claim 1, wherein X is —CH₂—,

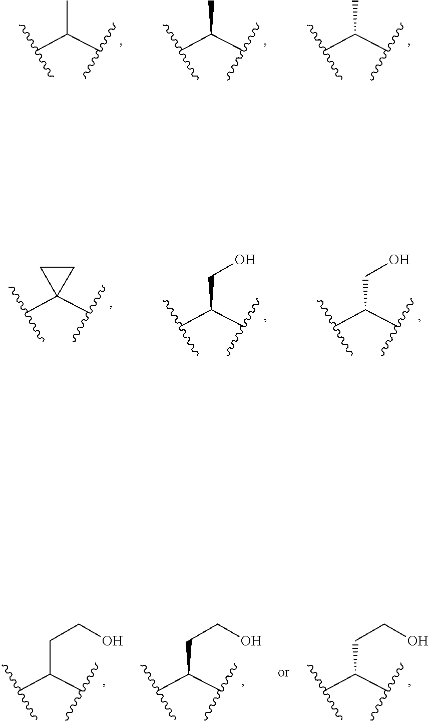

7. The compound of claim 1, wherein X² is

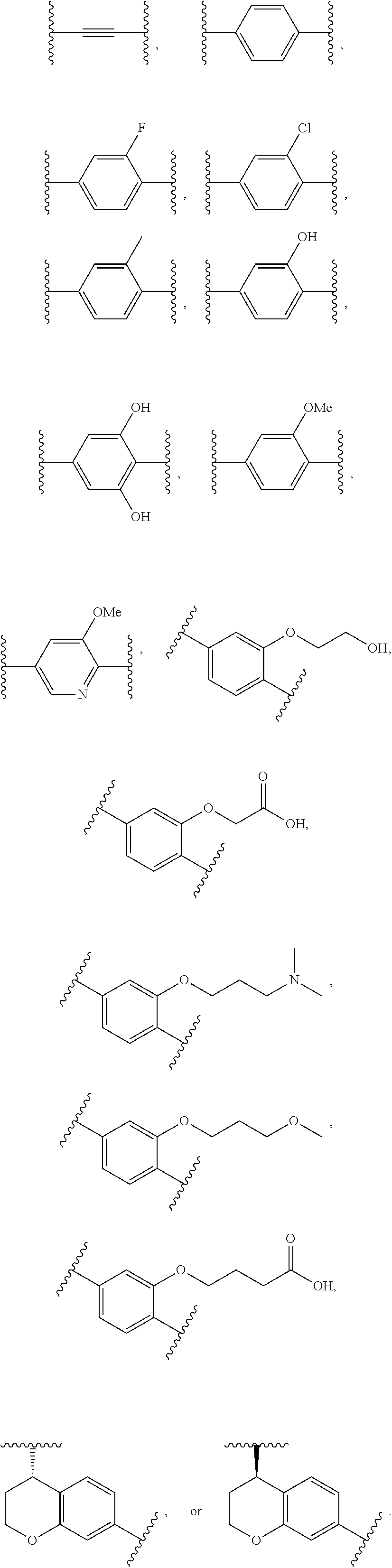

8-10. (canceled)

11. The compound of claim 1, wherein L a bivalent, saturated or unsaturated, straight or branched C₁₋₂₀ hydrocarbon chain, wherein 0-6 methylene units of L are independently replaced by -Cy-, —O—, —N(R)—, and —C(O)—.

12. The compound of claim 1, wherein L is -Cy-Cy-, —(CH₂)₁₋₁₀-Cy-Cy-, -Cy-(CH₂)₁₋₁₀-Cy-, -Cy-Cy-O—, —(CH₂)₁₋₁₀-Cy-Cy-O—, -Cy-(CH₂)₁₋₁₀-Cy-O—, -Cy-Cy-(CH₂)₁₋₁₀—O—, -Cy-Cy-CO—, —(CH₂)₁₋₁₀-Cy-Cy-CO—, -Cy-(CH₂)₁₋₁₀-Cy-CO—, -Cy-Cy-(CH₂)₁₋₁₀—CO—, -Cy-Cy-Cy-O—, -Cy-(CH₂)₁₋₁₀-Cy-Cy-O—, -Cy-Cy-(CH₂)₁₋₁₀-Cy-O—, -Cy-Cy-Cy-(CH₂)₁₋₁₀—O—, -Cy-Cy-Cy-CO—, -Cy-(CH₂)₁₋₁₀-Cy-Cy-CO—, -Cy-Cy-(CH₂)₁₋₁₀-Cy-CO—, or -Cy-Cy-Cy-(CH₂)₁₋₁₀—CO—.

13. The compound of claim 1, wherein L is selected from:

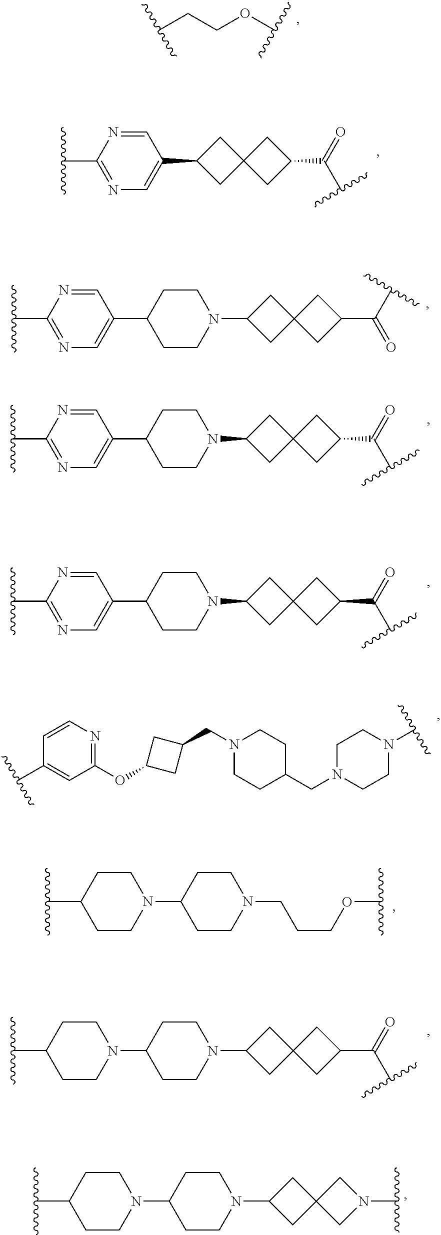

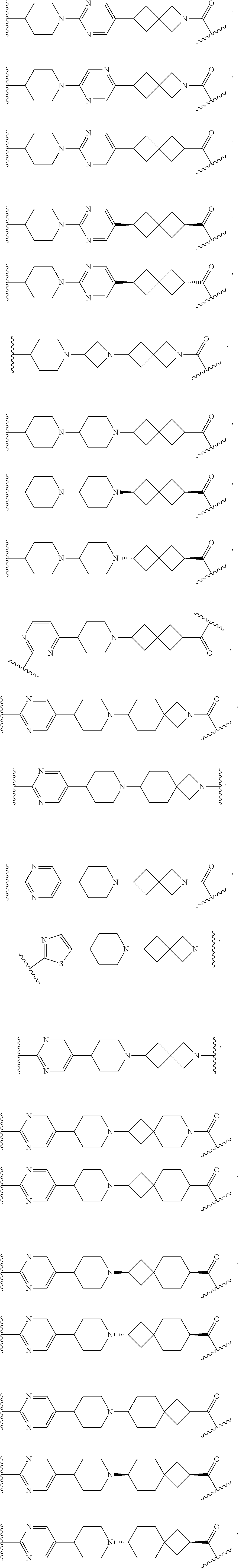

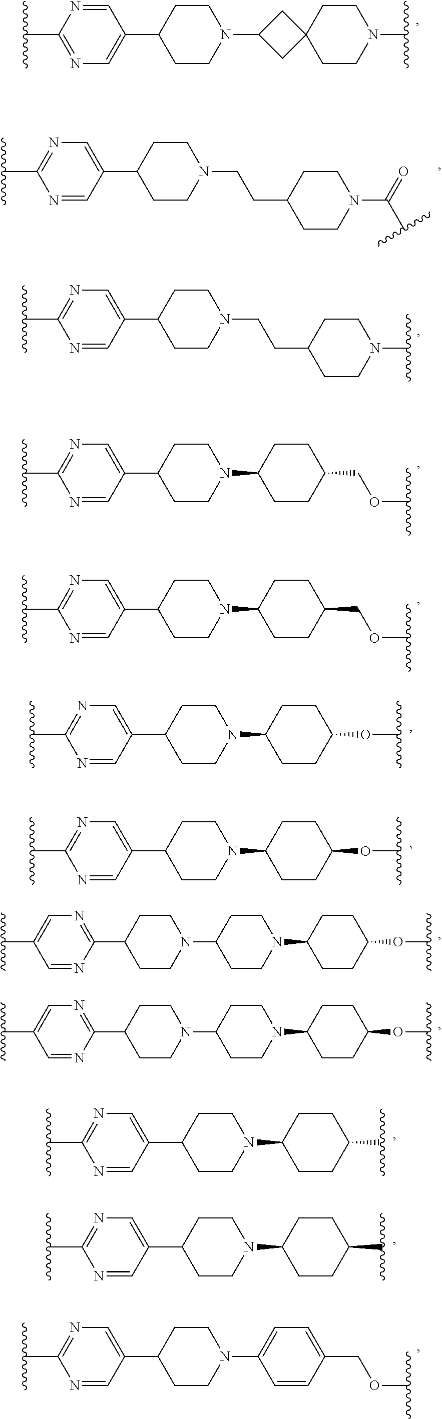

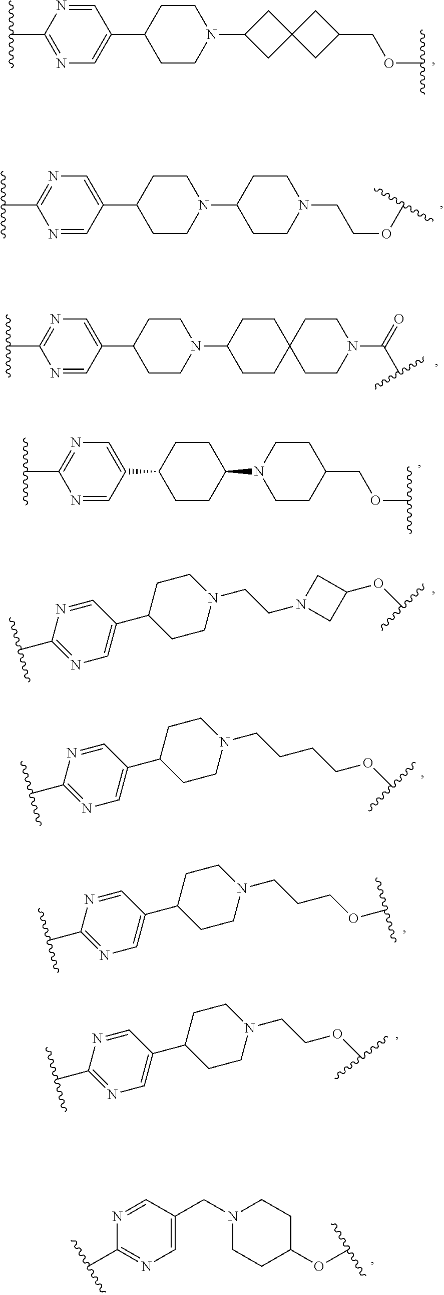

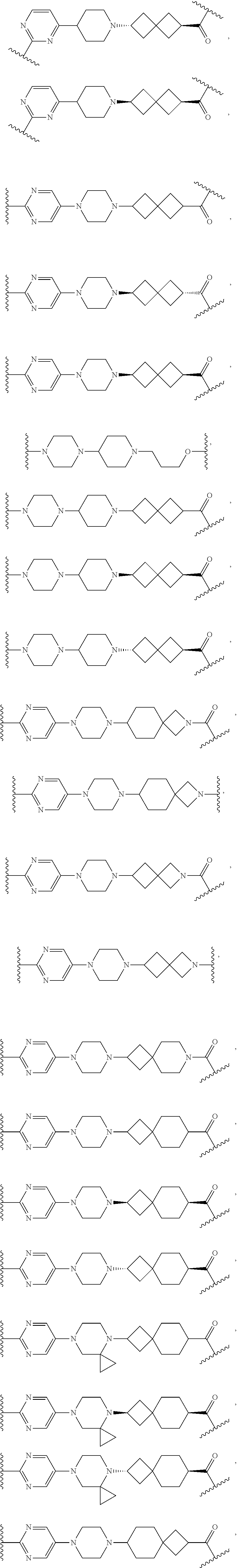

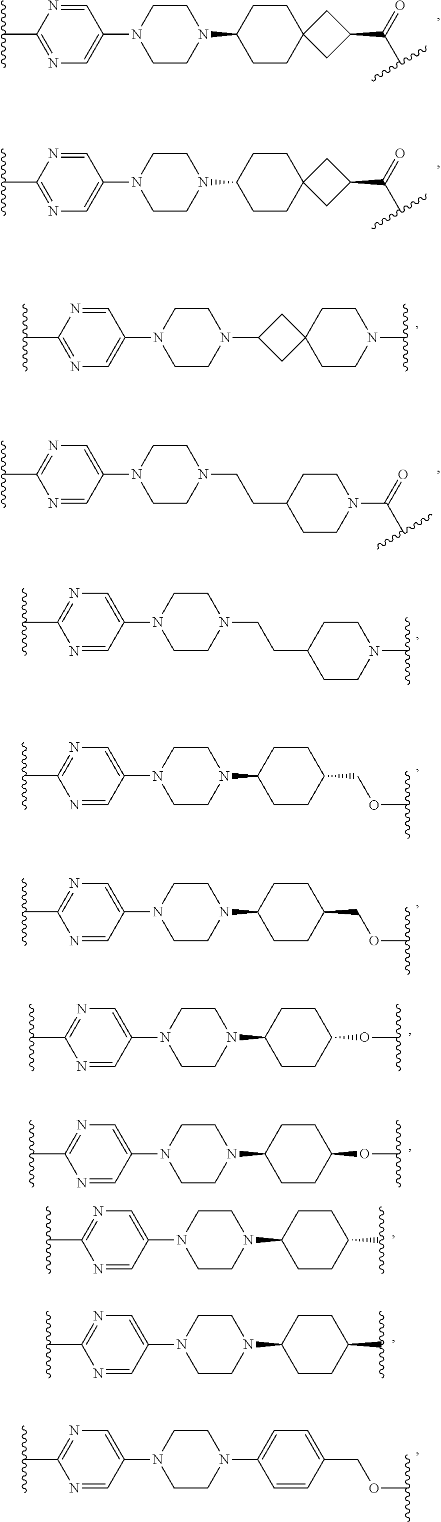

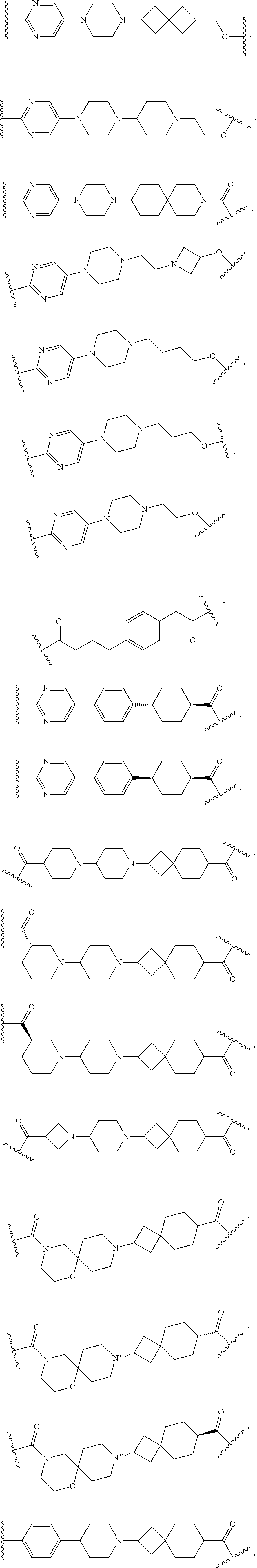

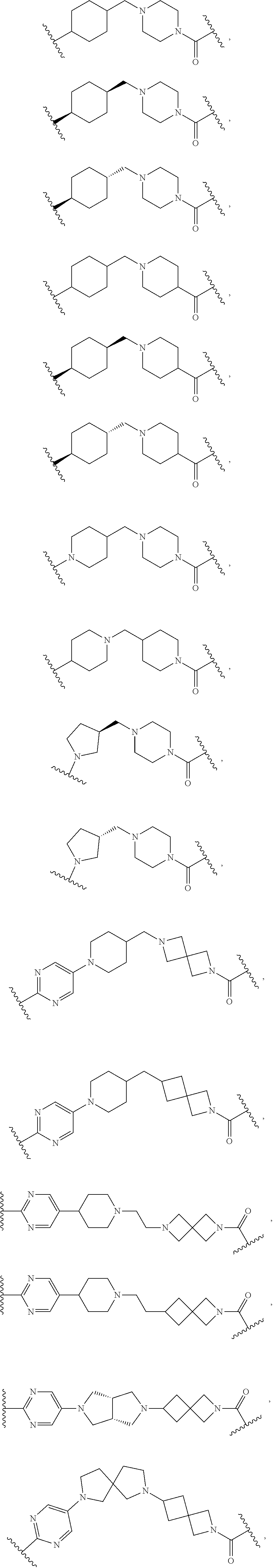

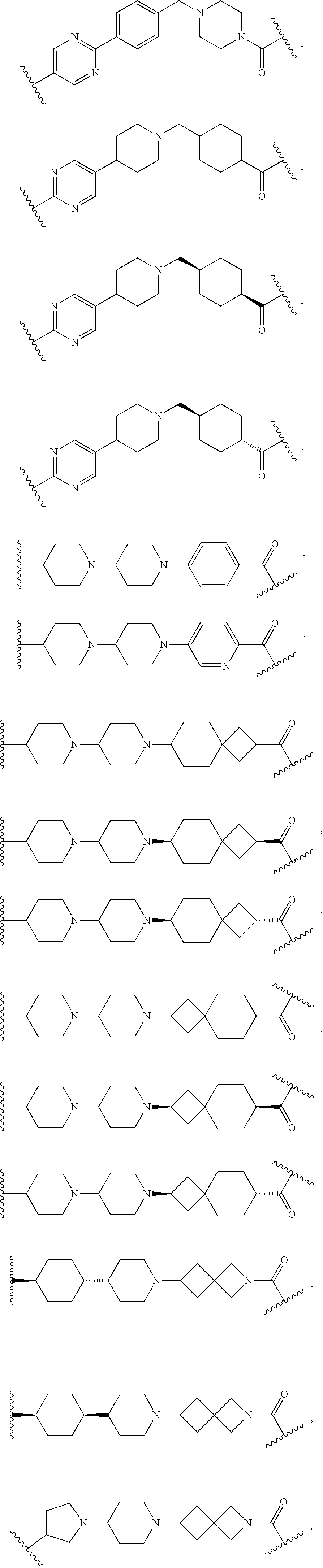

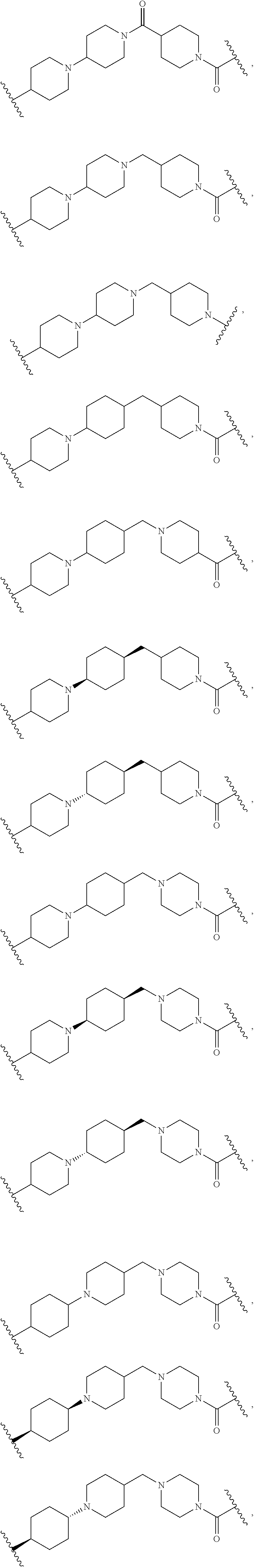

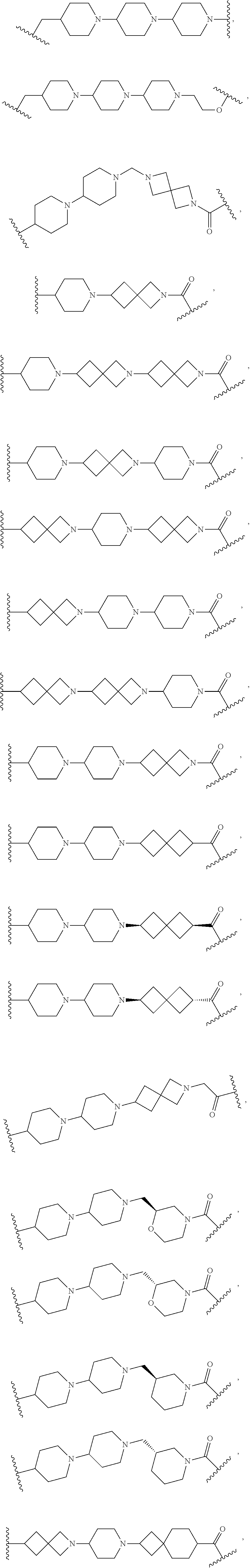

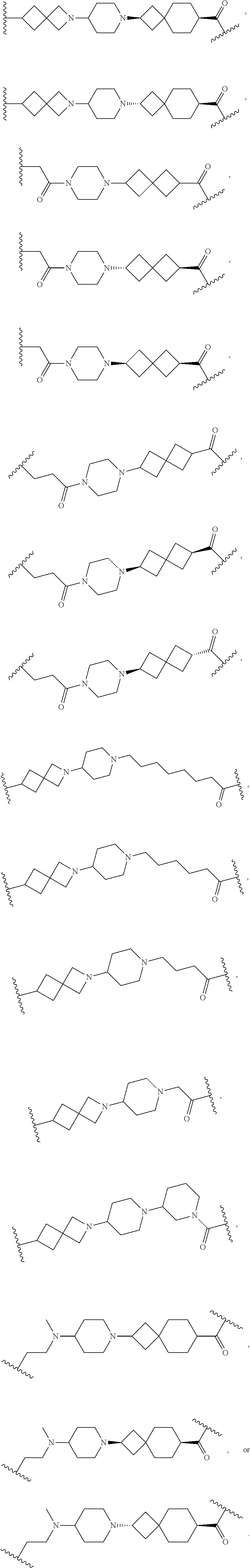

14. The compound of claim 1, wherein the compound is any one of the following formulae: 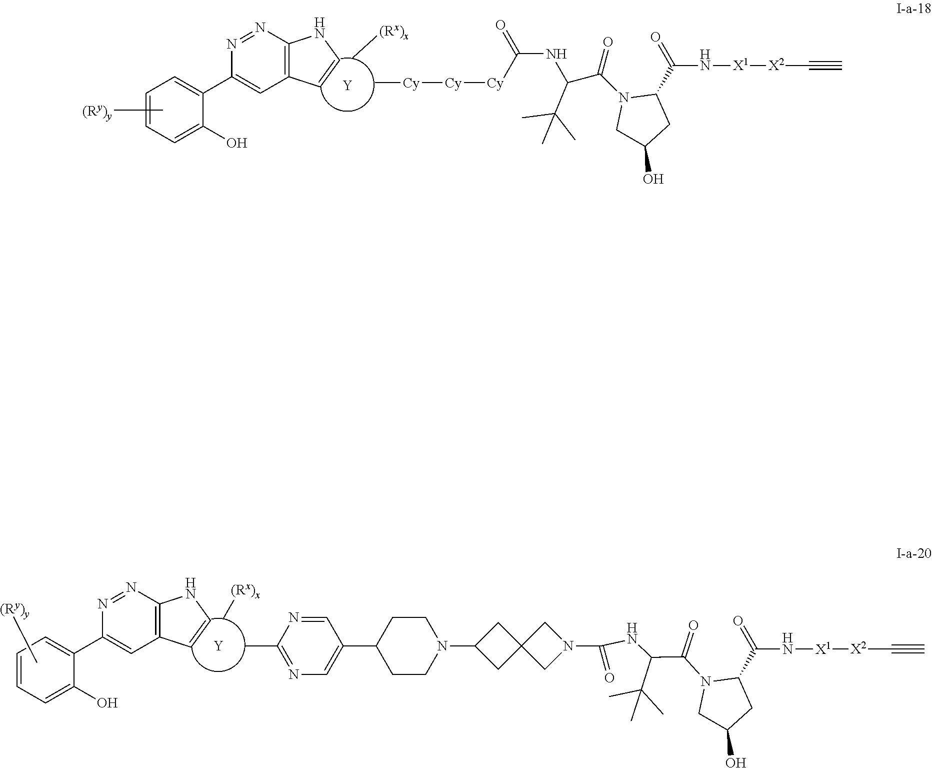
or a pharmaceutically acceptable salt thereof.

15. The compound of claim 1, wherein said compound is selected from: 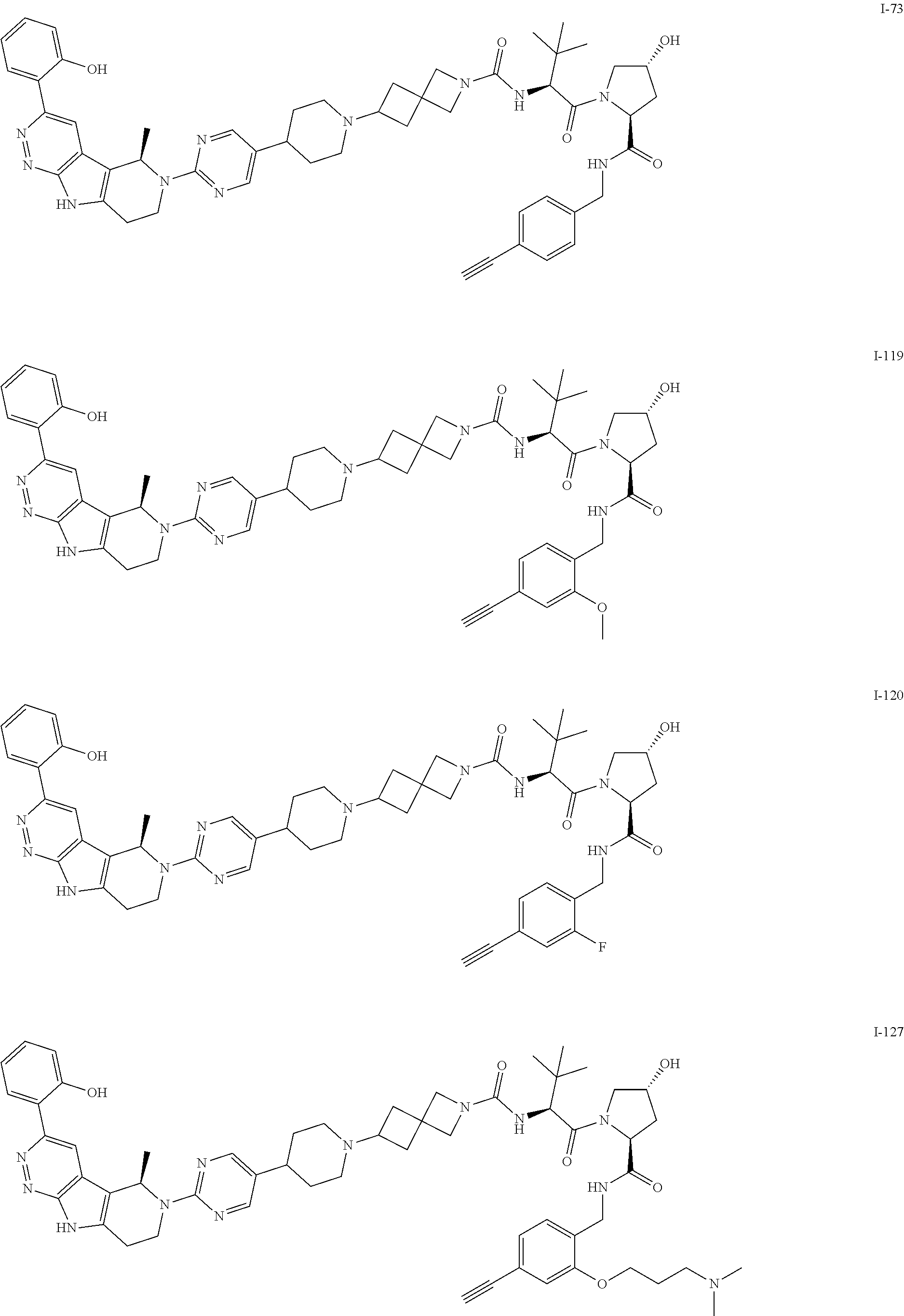 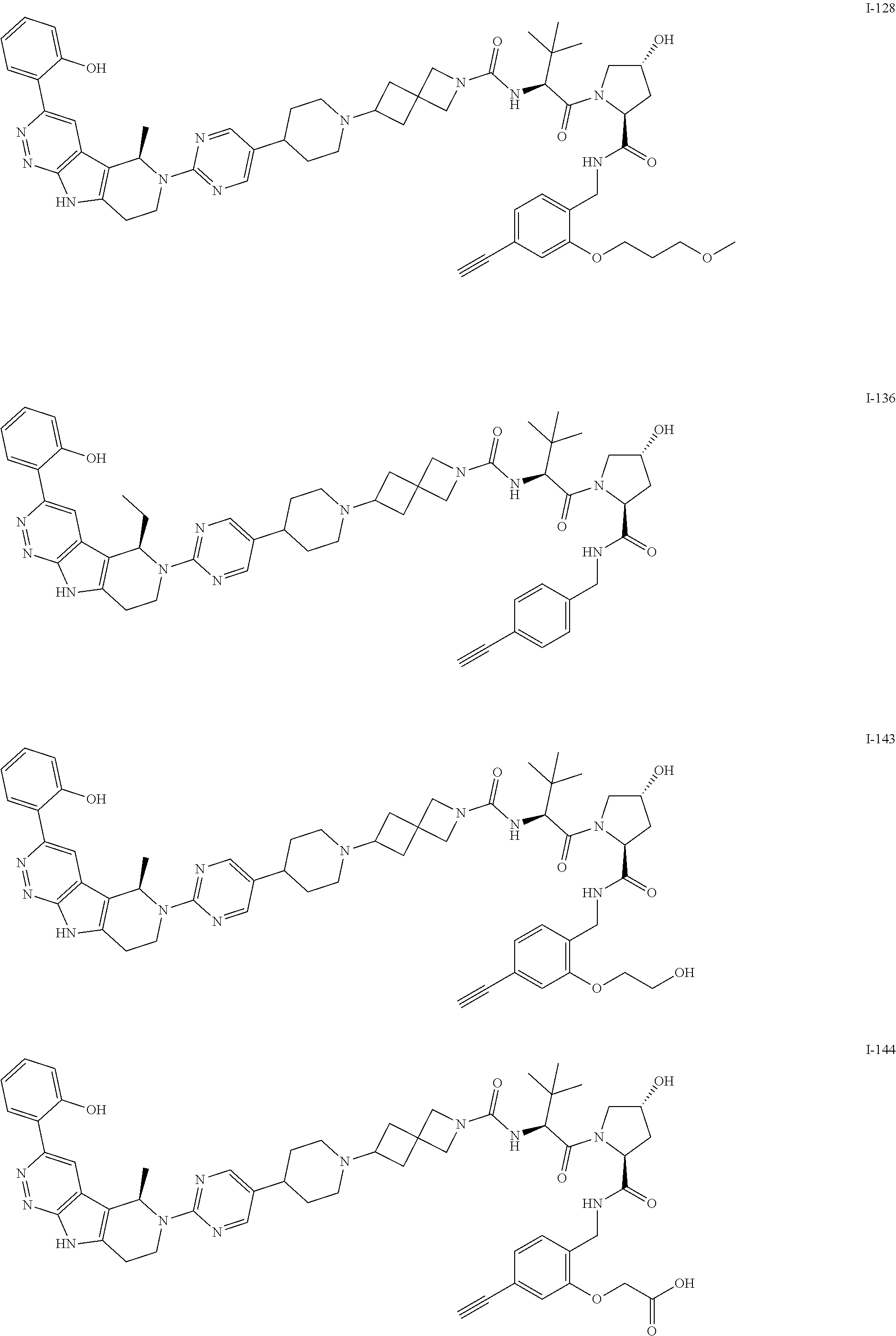 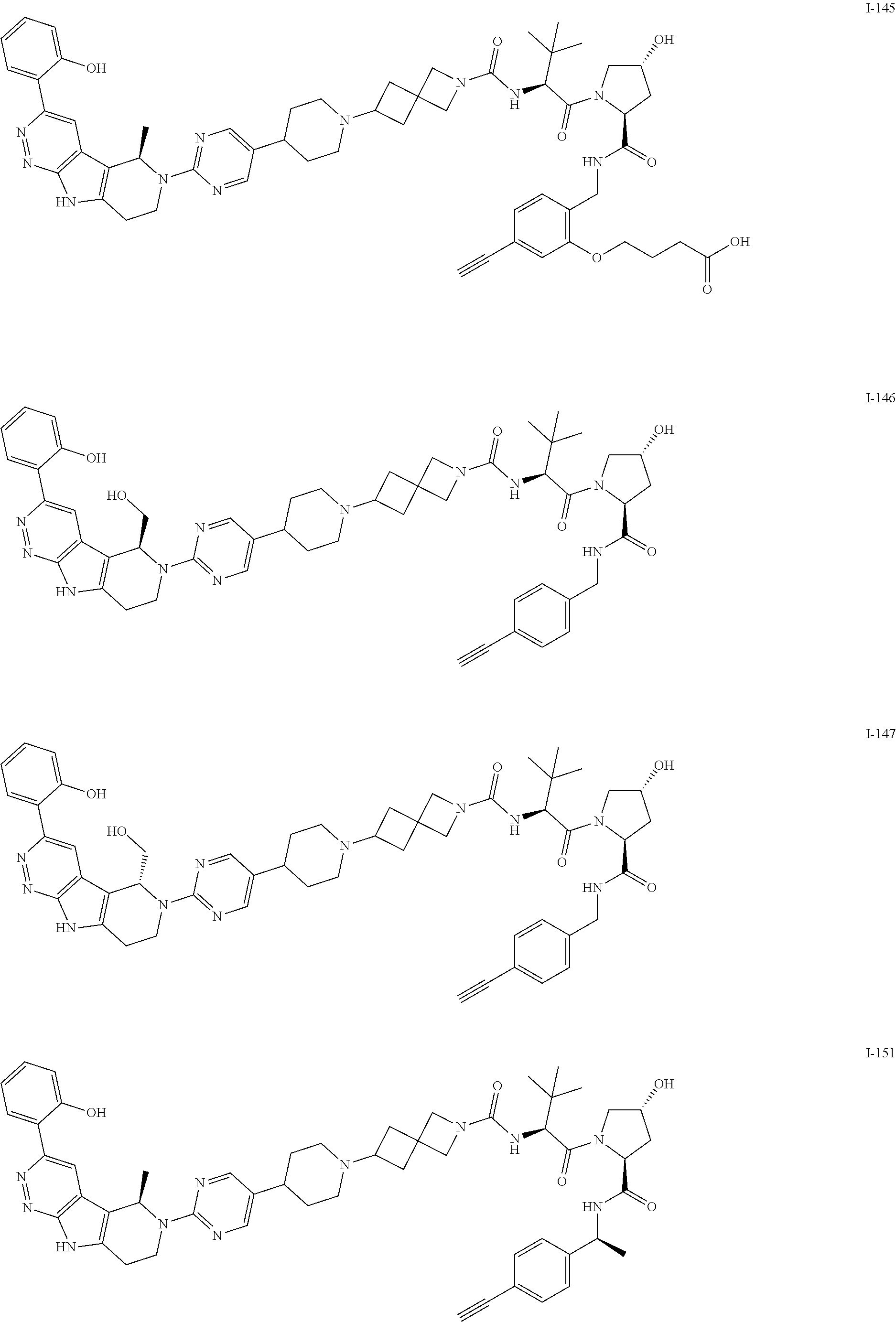 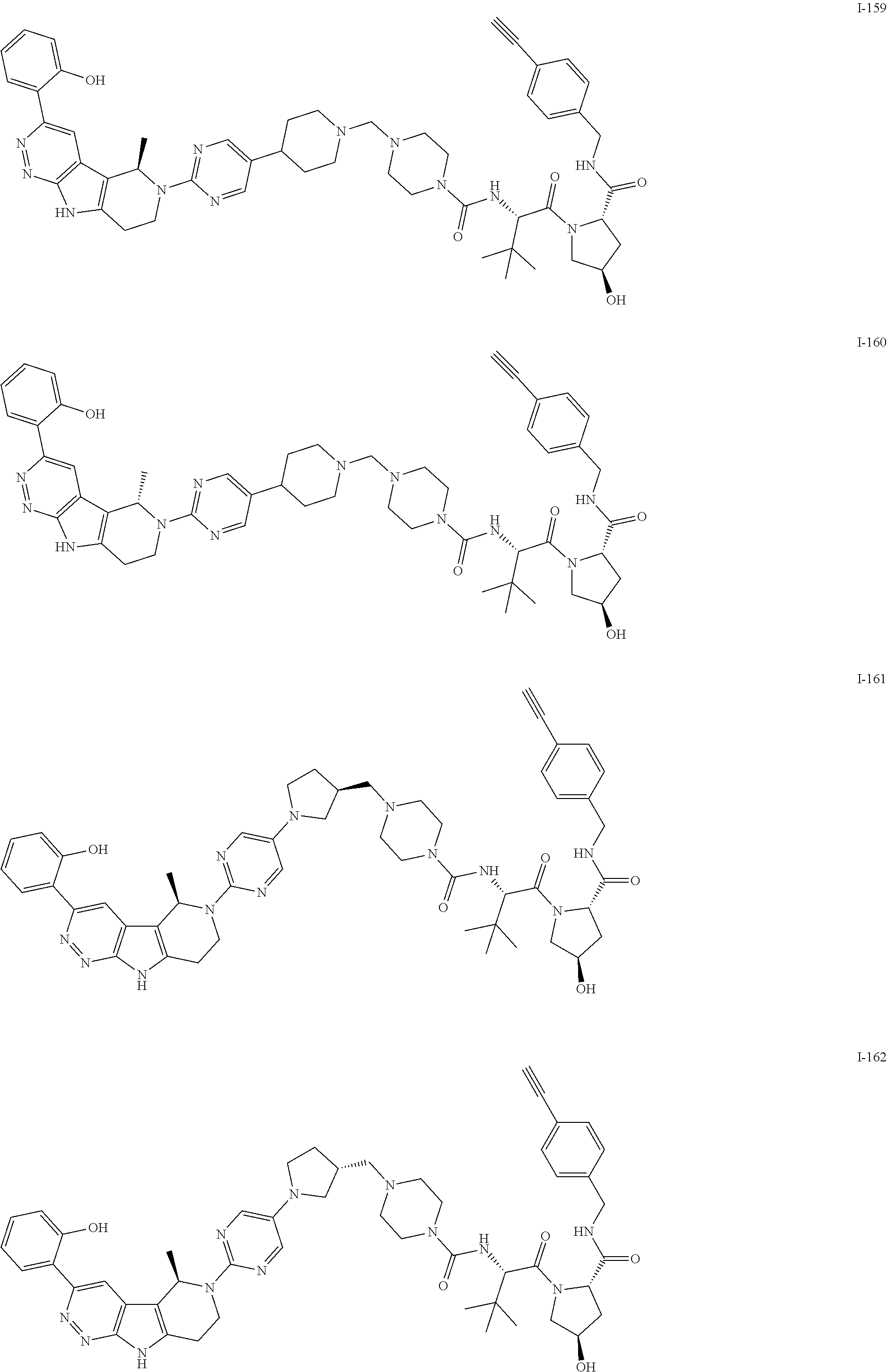 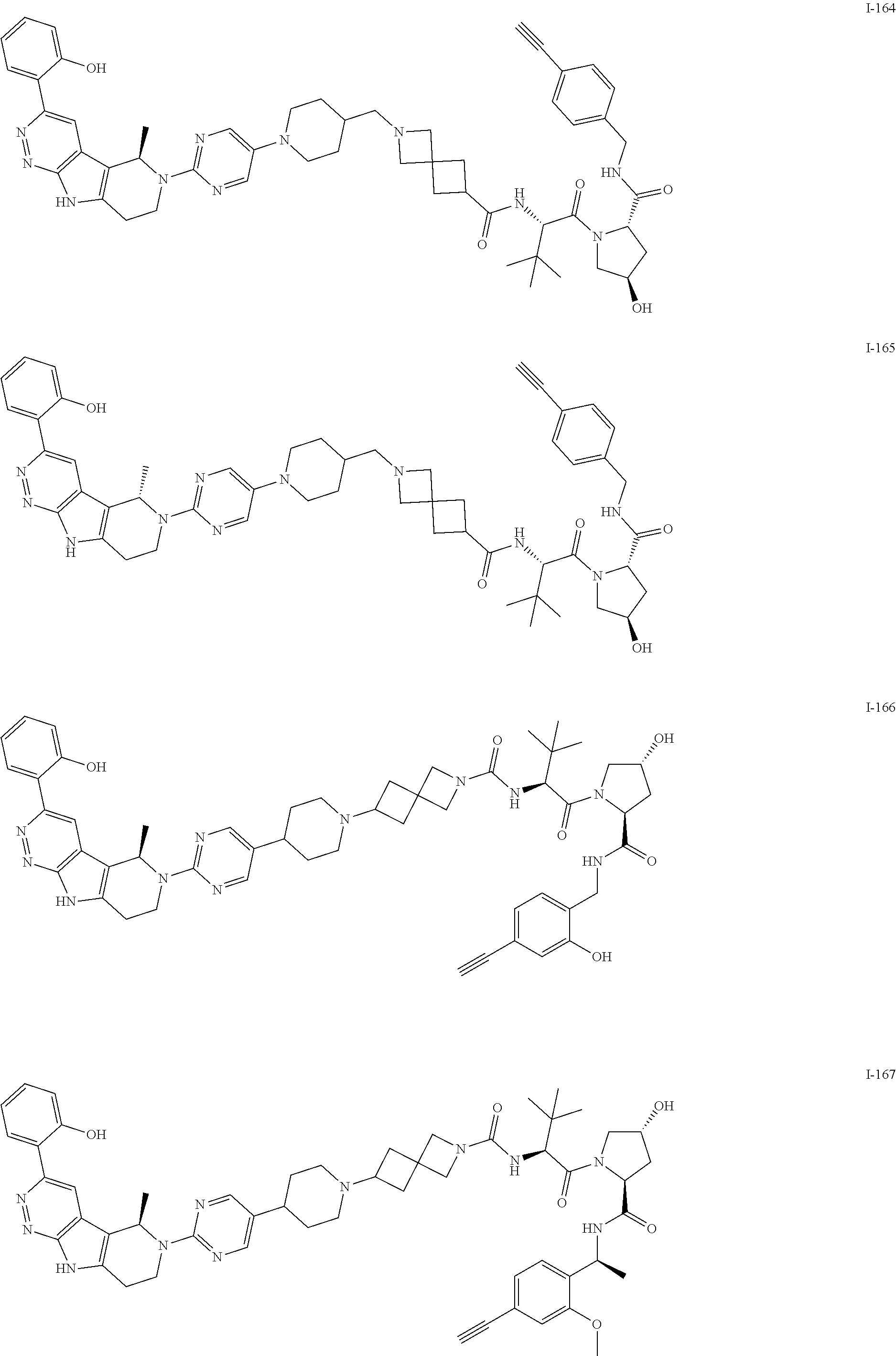 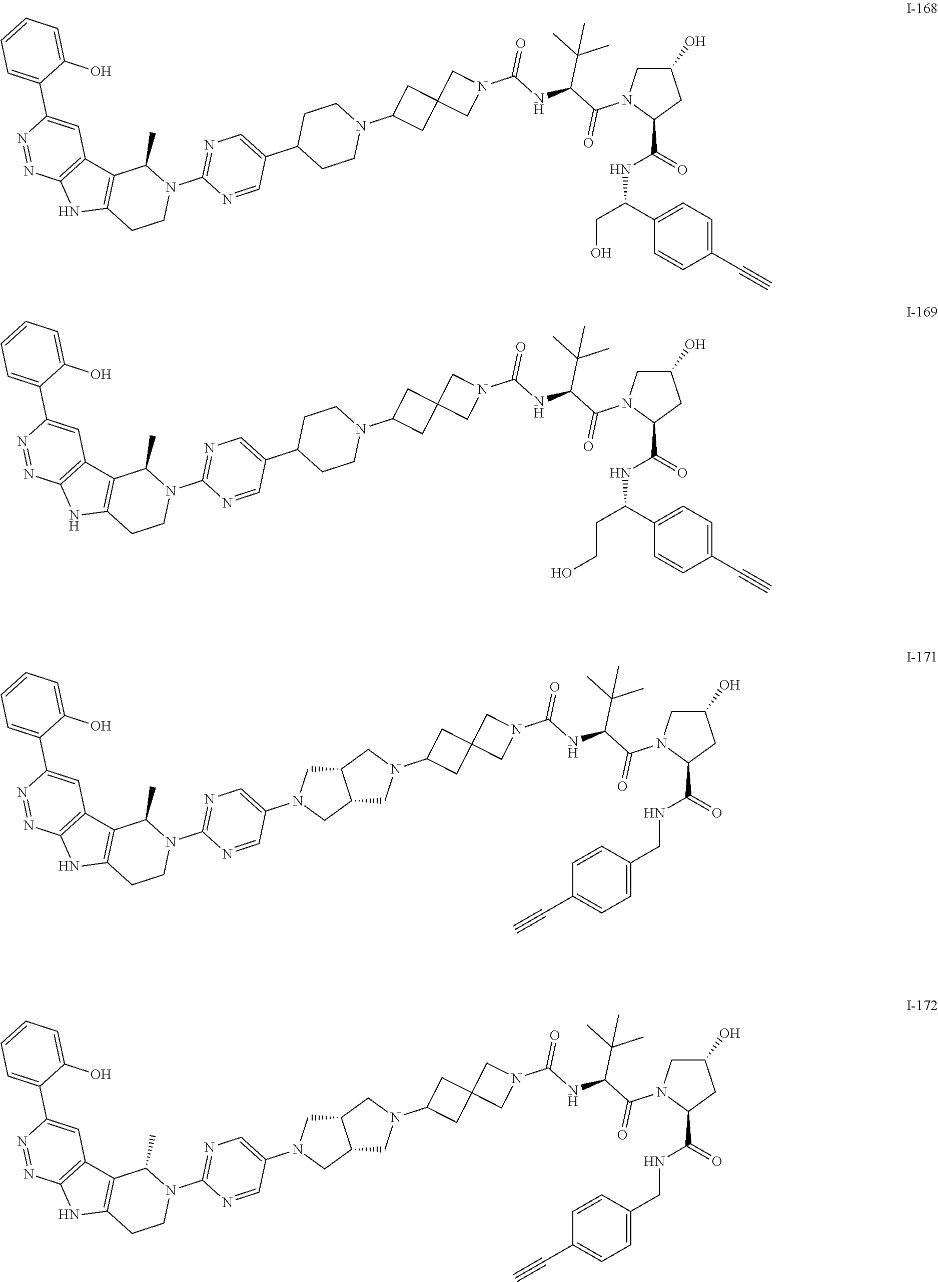 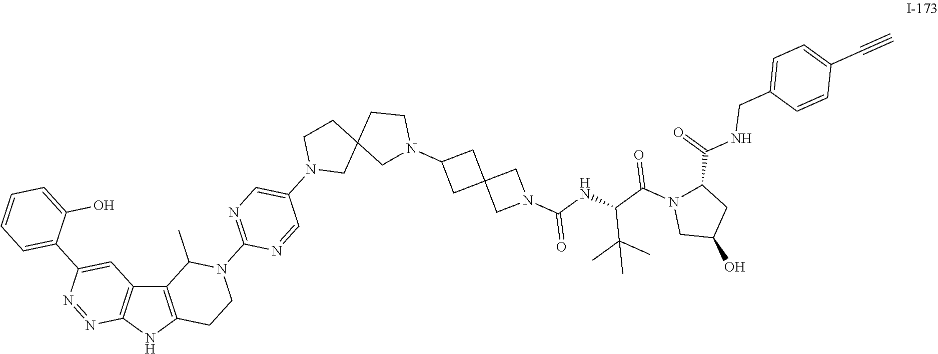 [SMILES: C#Cc1ccc(CNC(=O)[C@@H]2C[C@@H](O)CN2C(=O)[C@@H](NC(=O)N2CC3(CC(N4CCC5(CCN(c6cnc(N7CCc8[nH]c9nnc(-c%10ccccc%10O)cc9c8C7C)nc6)C5)C4)C3)C2)C(C)(C)C)cc1] 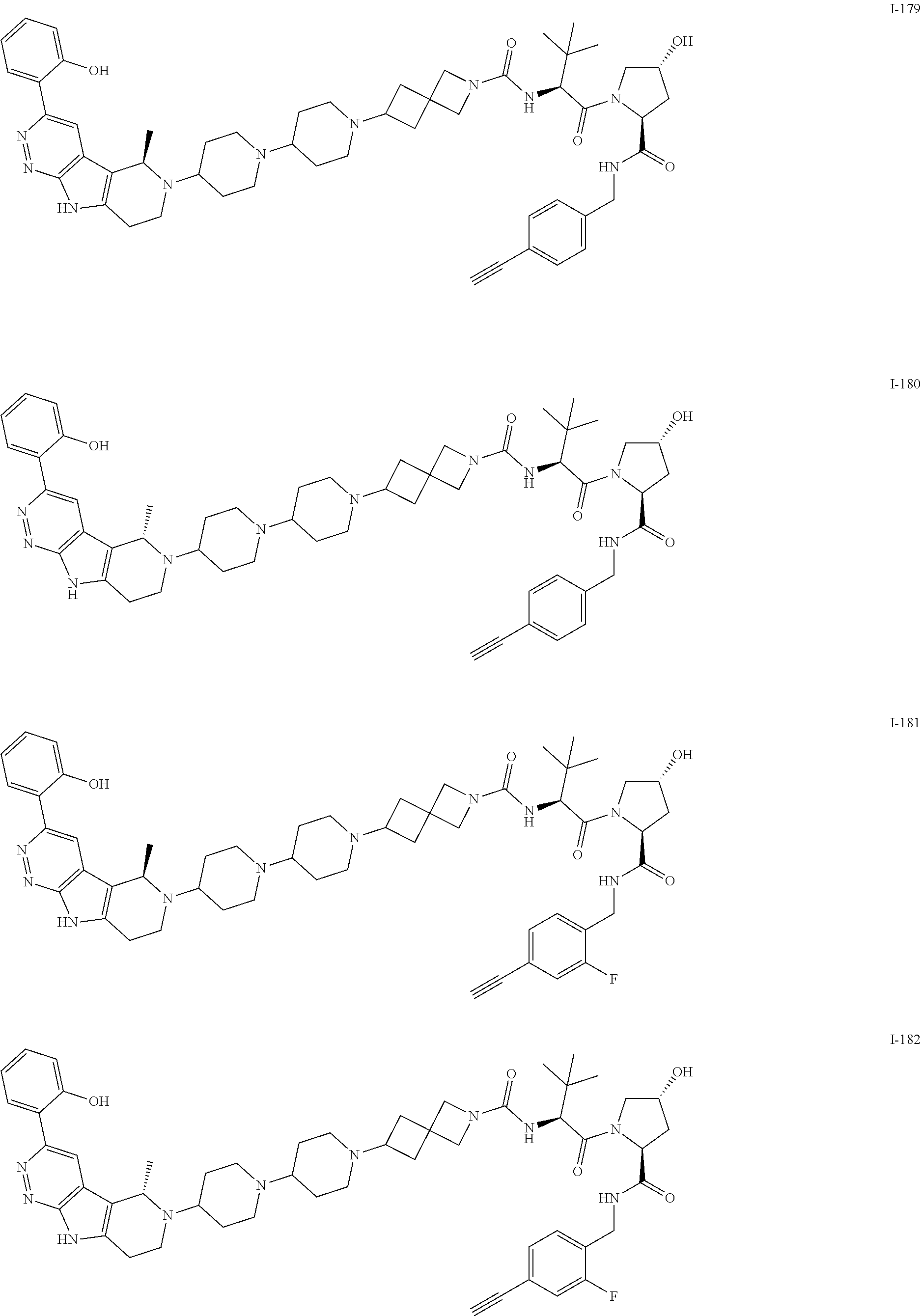 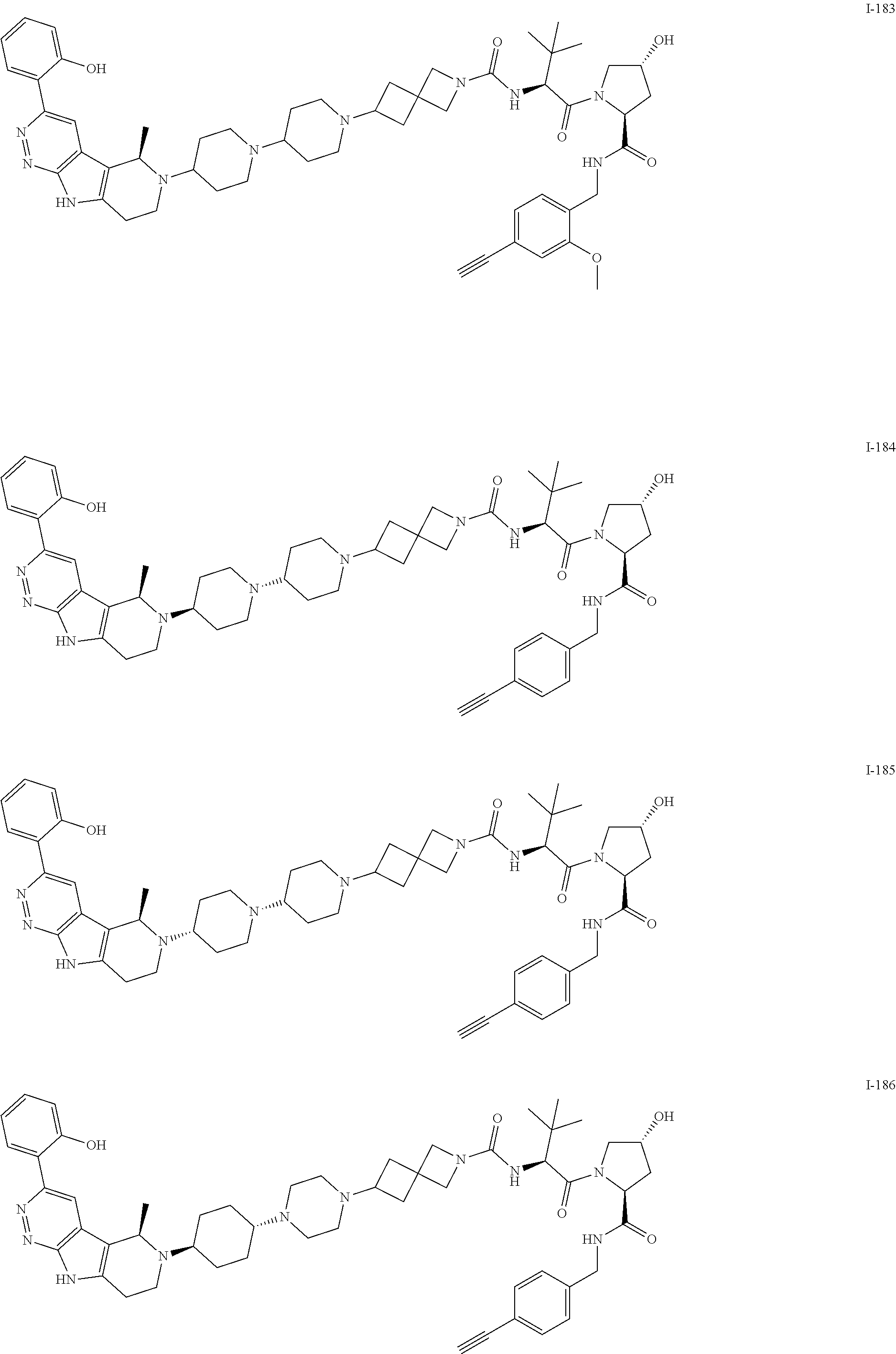 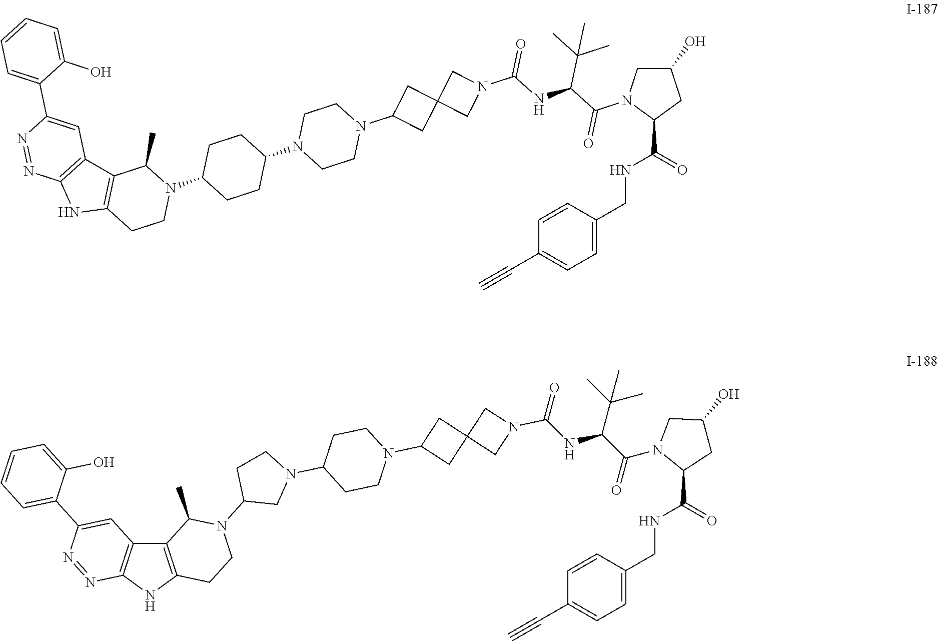 [SMILES: C#Cc1ccc(CNC(=O)[C@@H]2C[C@@H](O)CN2C(=O)[C@@H](NC(=O)N2CC3(CC(N4CCN([C@H]5CC[C@@H](N6CCc7[nH]c8nnc(-c9ccccc9O)cc8c7[C@H]6C)CC5)CC4)C3)C2)C(C)(C)C)cc1 ; C#Cc1ccc(CNC(=O)[C@@H]2C[C@@H](O)CN2C(=O)[C@@H](NC(=O)N2CC3(CC(N4CCC(N5CCC(N6CCc7[nH]c8nnc(-c9ccccc9O)cc8c7[C@H]6C)C5)CC4)C3)C2)C(C)(C)C)cc1] 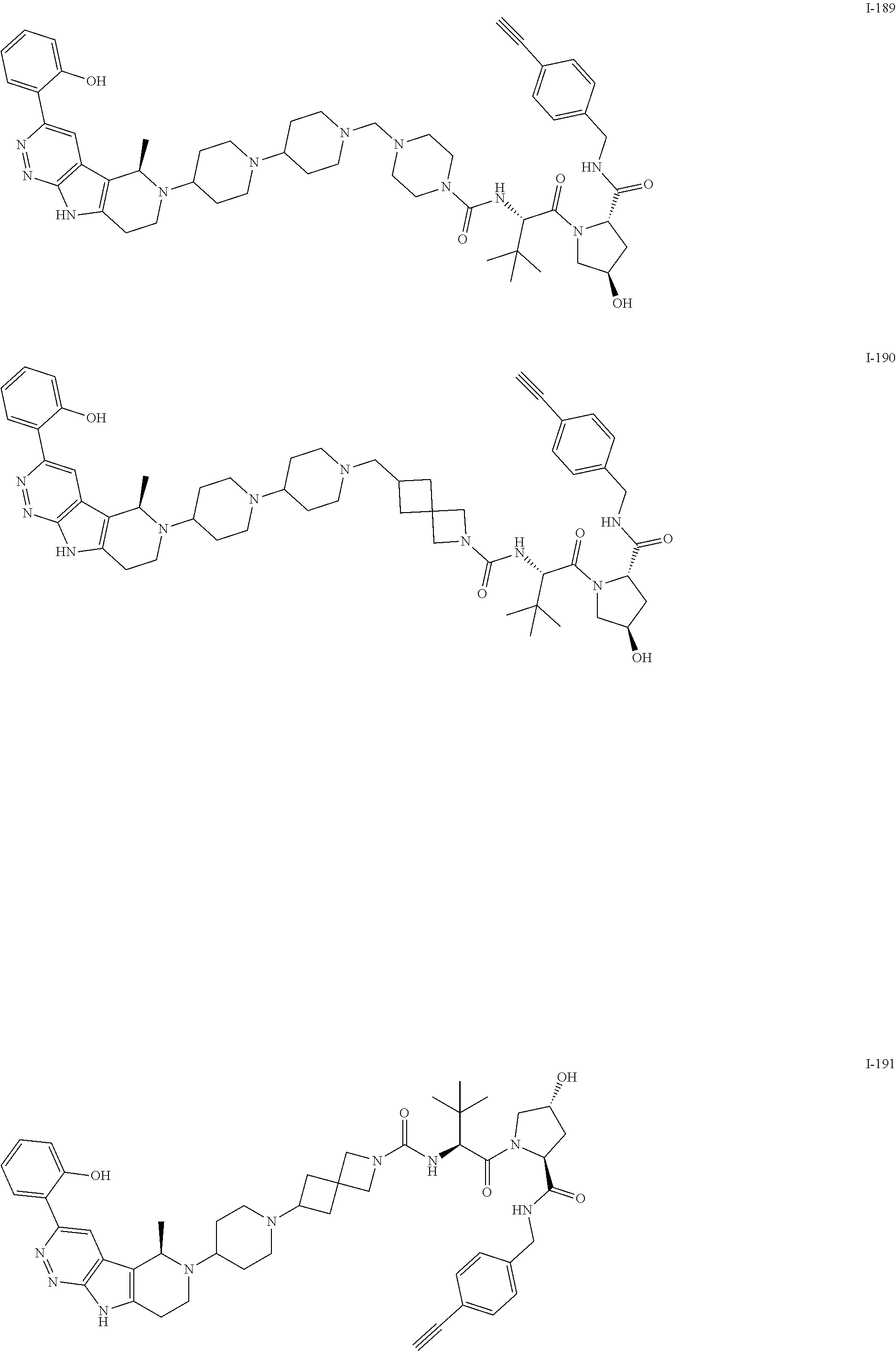 [SMILES: C#Cc1ccc(CNC(=O)[C@@H]2C[C@@H](O)CN2C(=O)[C@@H](NC(=O)N2CCN(CN3CCC(N4CCC(N5CCc6[nH]c7nnc(-c8ccccc8O)cc7c6[C@H]5C)CC4)CC3)CC2)C(C)(C)C)cc1 ; C#Cc1ccc(CNC(=O)[C@@H]2C[C@@H](O)CN2C(=O)[C@@H](NC(=O)N2CC3(CC(CN4CCC(N5CCC(N6CCc7[nH]c8nnc(-c9ccccc9O)cc8c7[C@H]6C)CC5)CC4)C3)C2)C(C)(C)C)cc1 ; C#Cc1ccc(CNC(=O)[C@@H]2C[C@@H](O)CN2C(=O)[C@@H](NC(=O)N2CC3(CC(N4CCC(N5CCc6[nH]c7nnc(-c8ccccc8O)cc7c6[C@H]5C)CC4)C3)C2)C(C)(C)C)cc1] 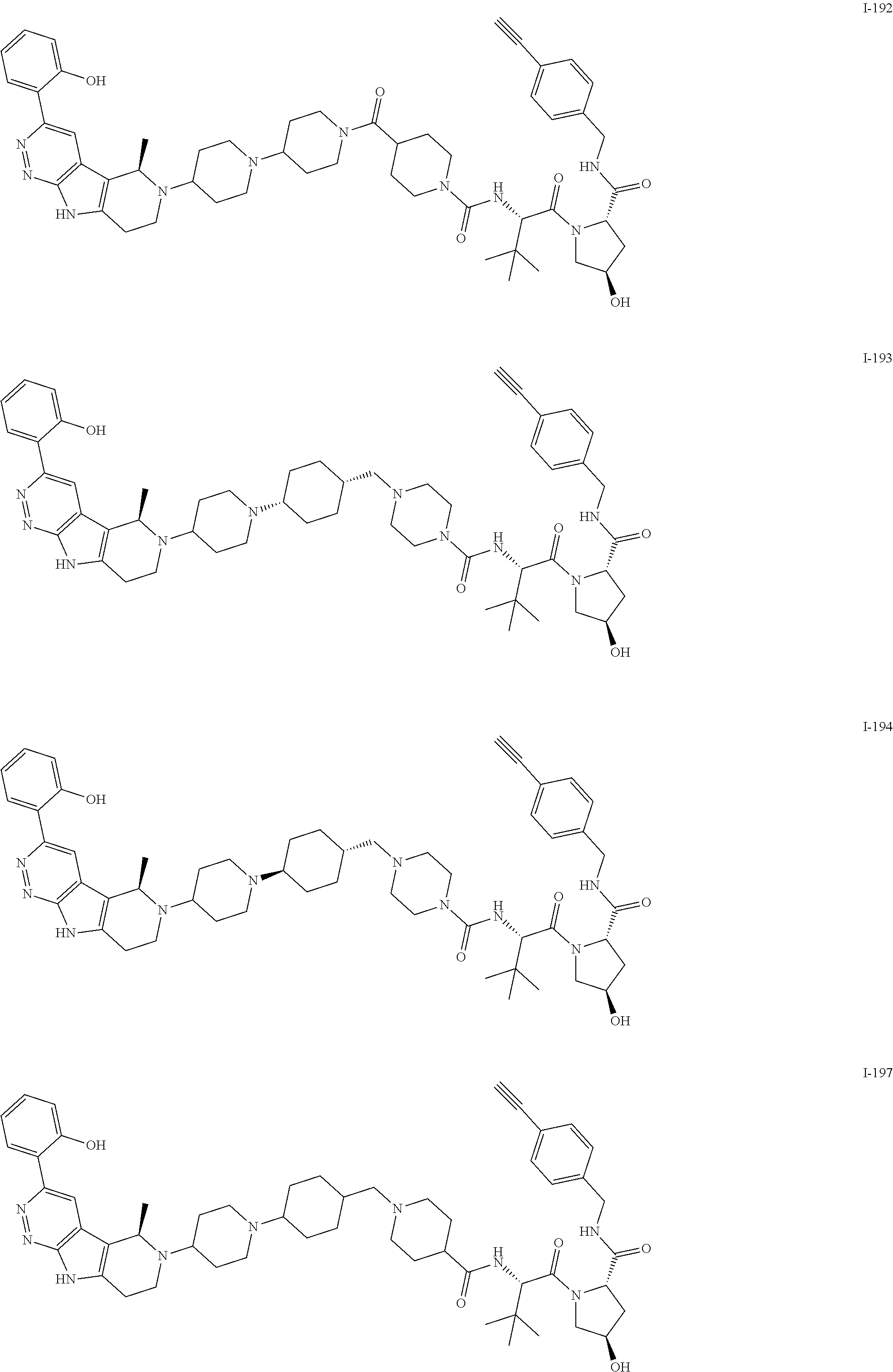 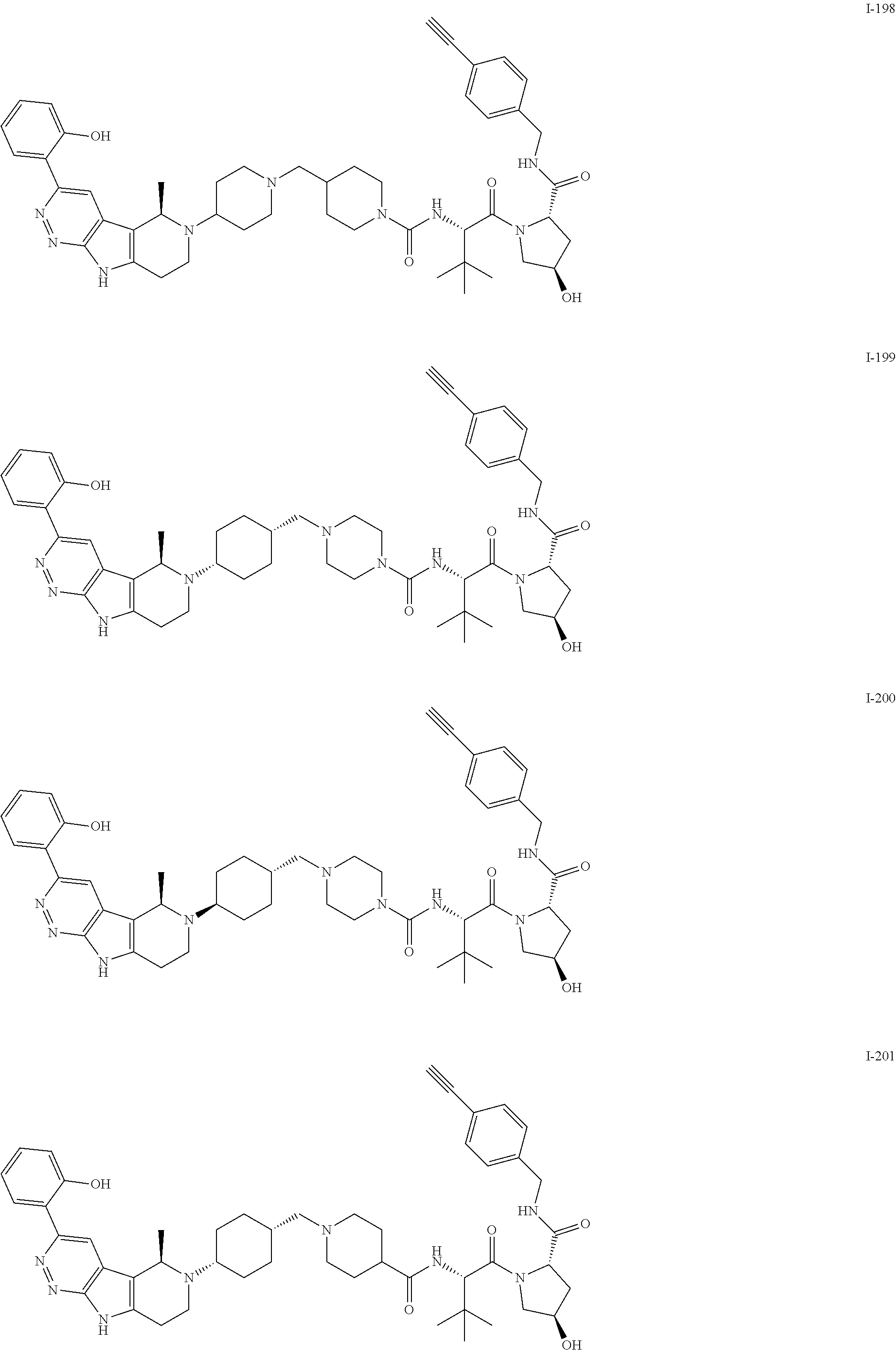 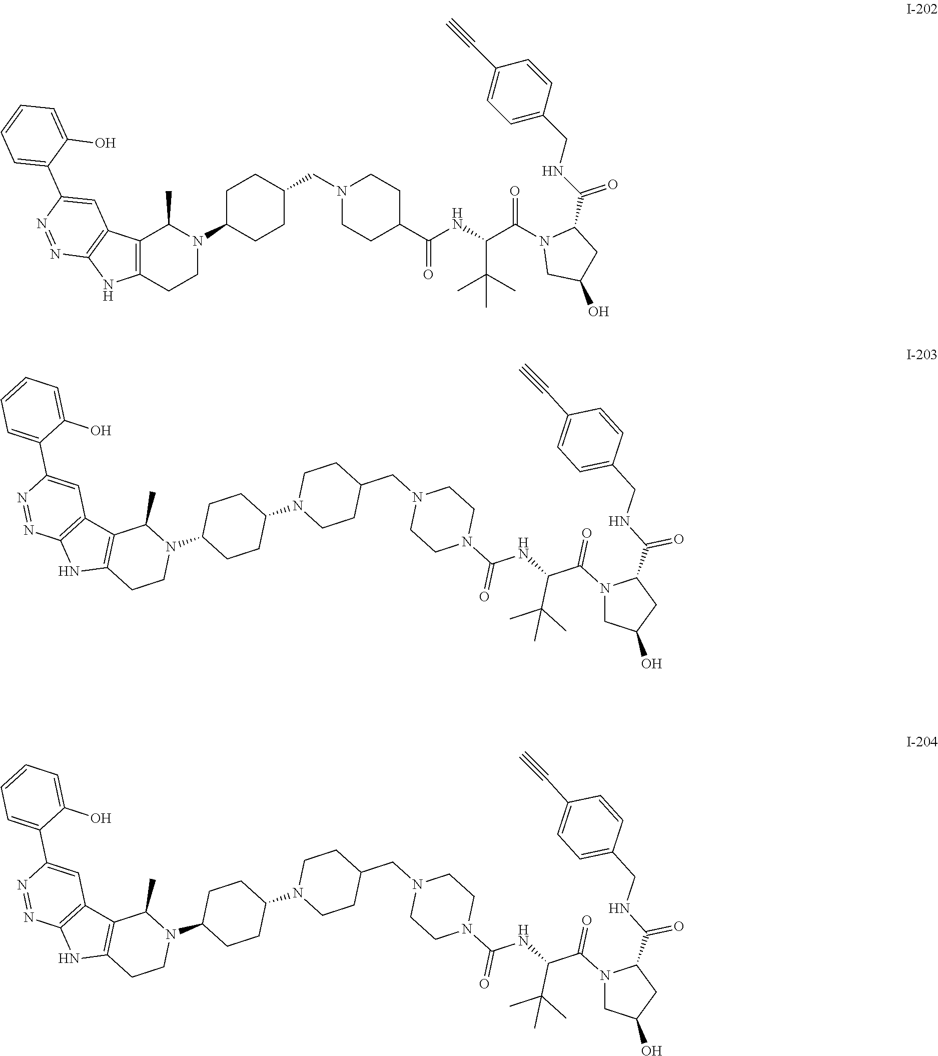 [SMILES: C#Cc1ccc(CNC(=O)[C@@H]2C[C@@H](O)CN2C(=O)[C@@H](NC(=O)C2CCN(C[C@H]3CC[C@H](N4CCc5[nH]c6nnc(-c7ccccc7O)cc6c5[C@H]4C)CC3)CC2)C(C)(C)C)cc1 ; C#Cc1ccc(CNC(=O)[C@@H]2C[C@@H](O)CN2C(=O)[C@@H](NC(=O)N2CCN(CC3CCN([C@H]4CC[C@@H](N5CCc6[nH]c7nnc(-c8ccccc8O)cc7c6[C@H]5C)CC4)CC3)CC2)C(C)(C)C)cc1 ; C#Cc1ccc(CNC(=O)[C@@H]2C[C@@H](O)CN2C(=O)[C@@H](NC(=O)N2CCN(CC3CCN([C@H]4CC[C@H](N5CCc6[nH]c7nnc(-c8ccccc8O)cc7c6[C@H]5C)CC4)CC3)CC2)C(C)(C)C)cc1] 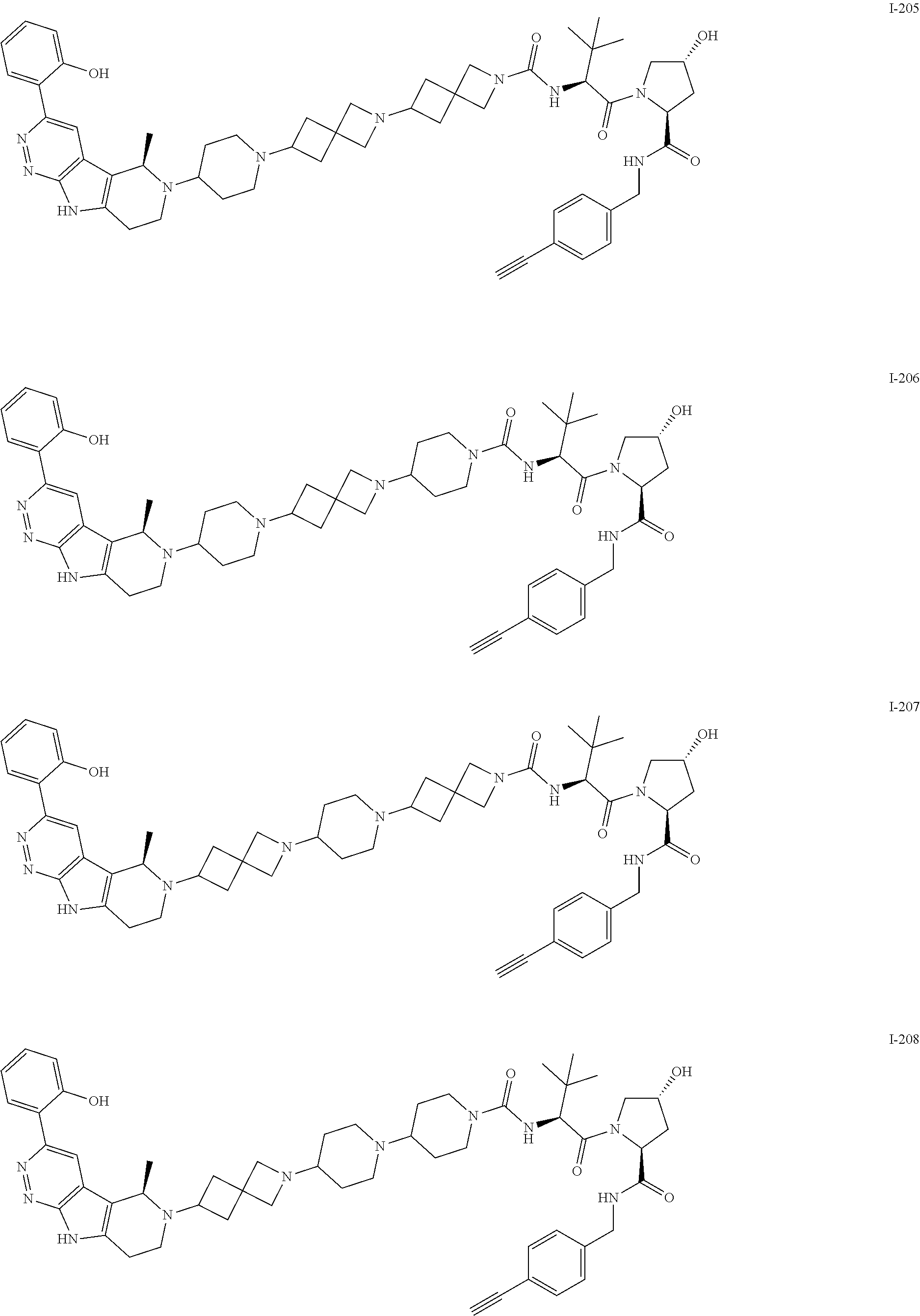 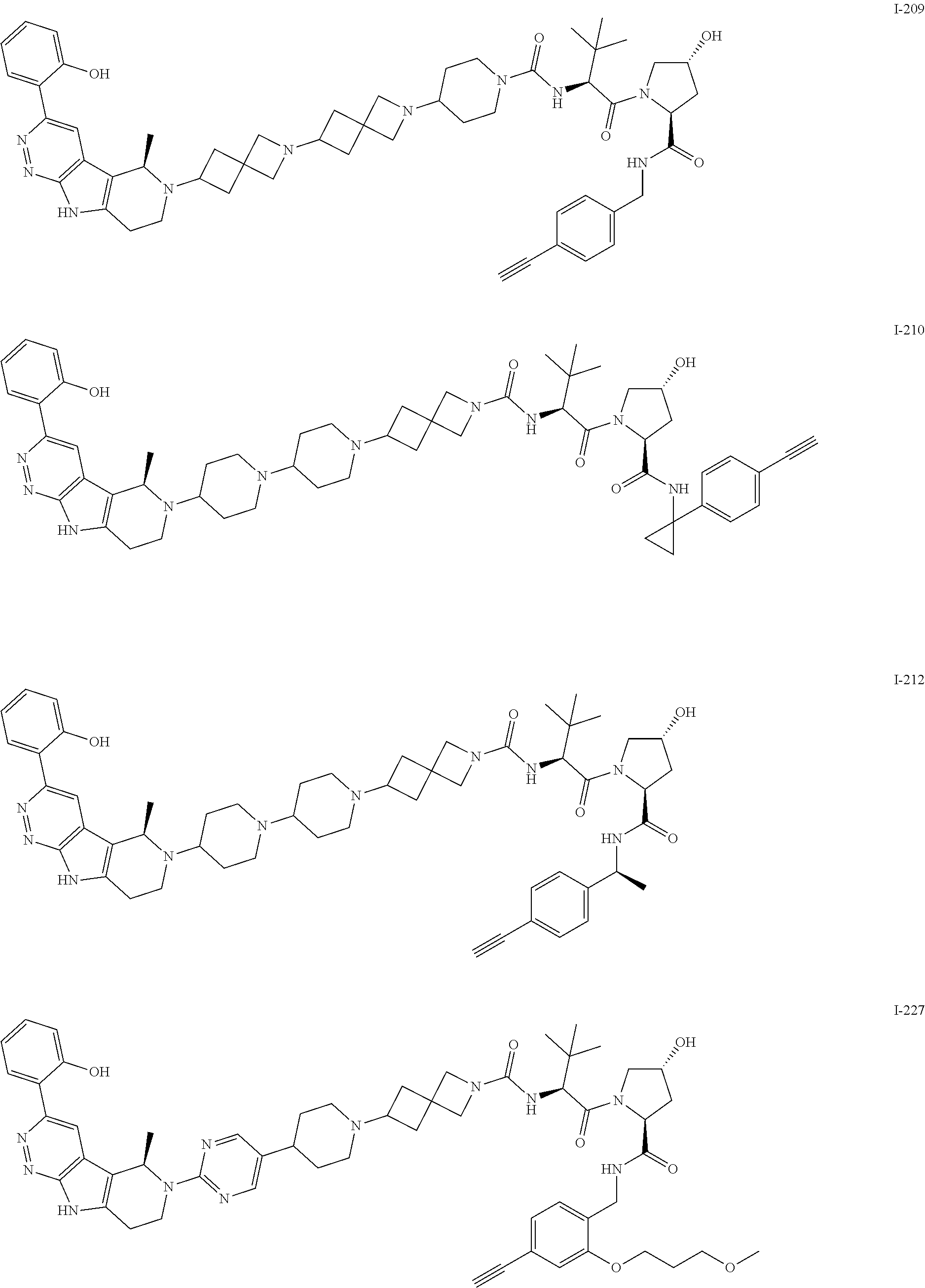 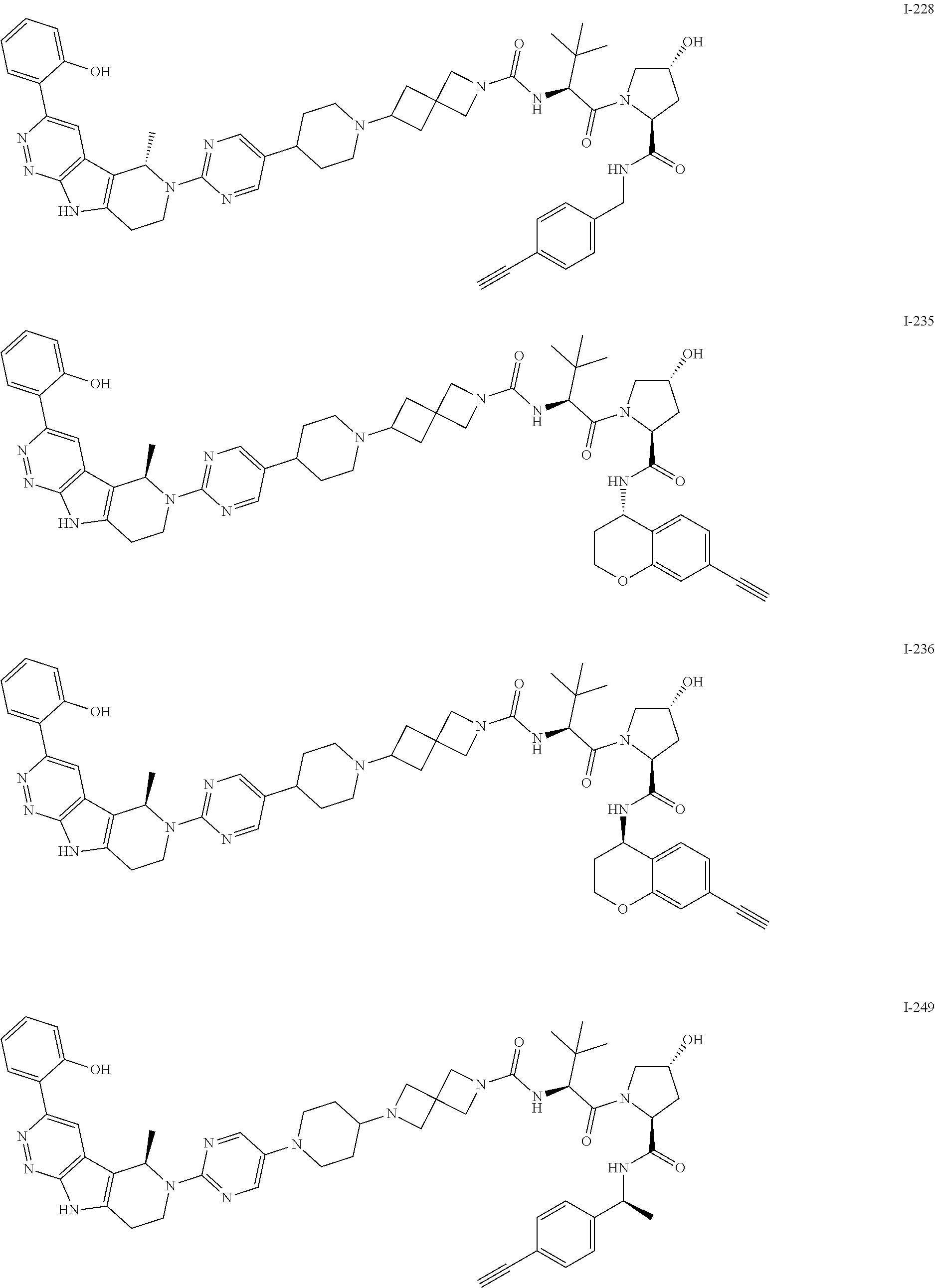 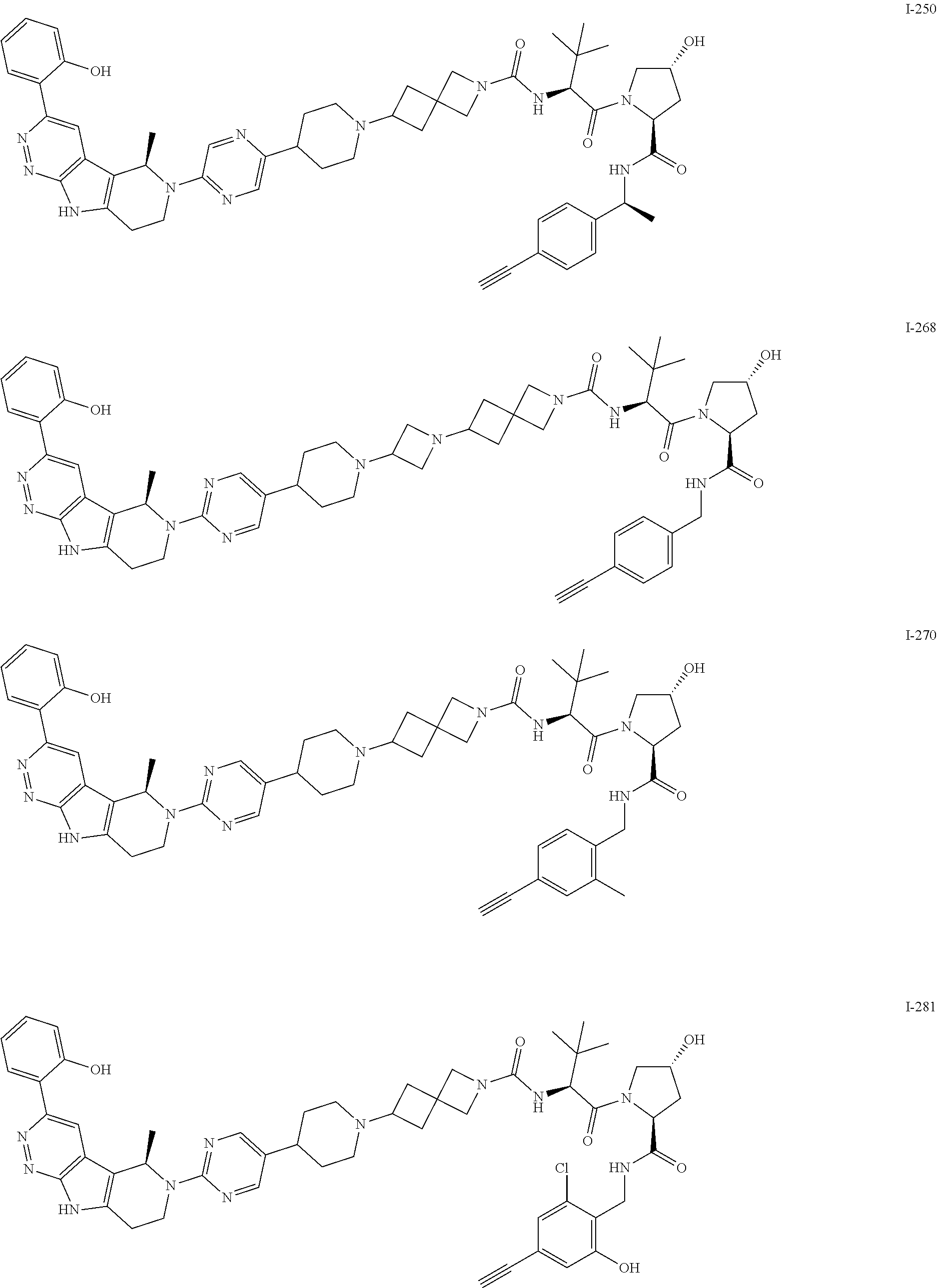 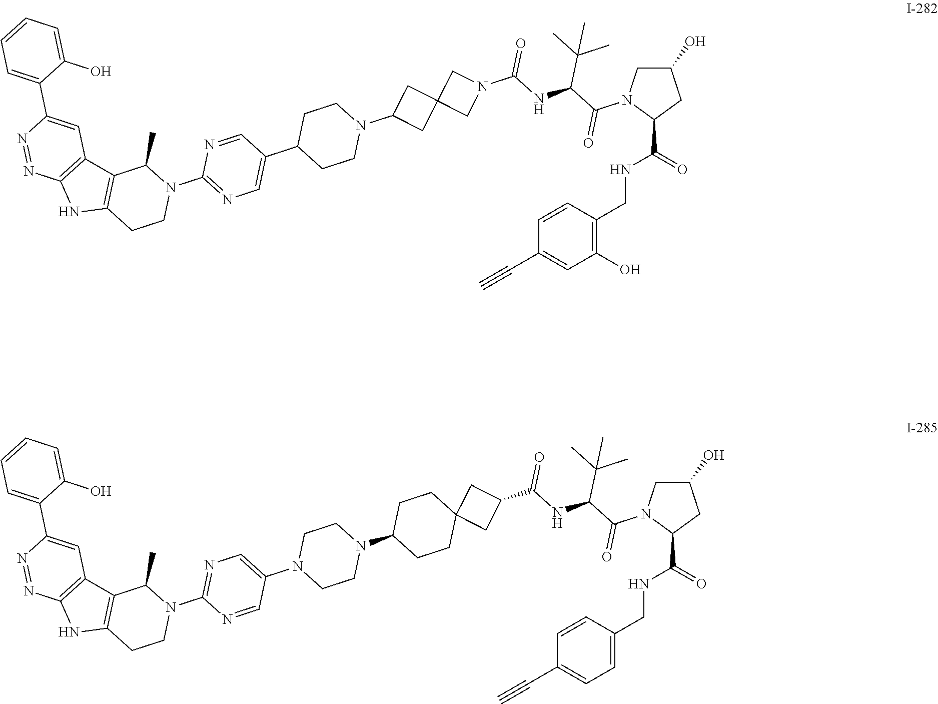 [SMILES: C#Cc1ccc(CNC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C)nc5)CC4)C3)C2)C(C)(C)C)c(O)c1 ; C#Cc1ccc(CNC(=O)[C@@H]2C[C@@H](O)CN2C(=O)[C@@H](NC(=O)[C@H]2CC3(CC[C@H](N4CCN(c5cnc(N6CCc7[nH]c8nnc(-c9ccccc9O)cc8c7[C@H]6C)nc5)CC4)CC3)C2)C(C)(C)C)cc1] 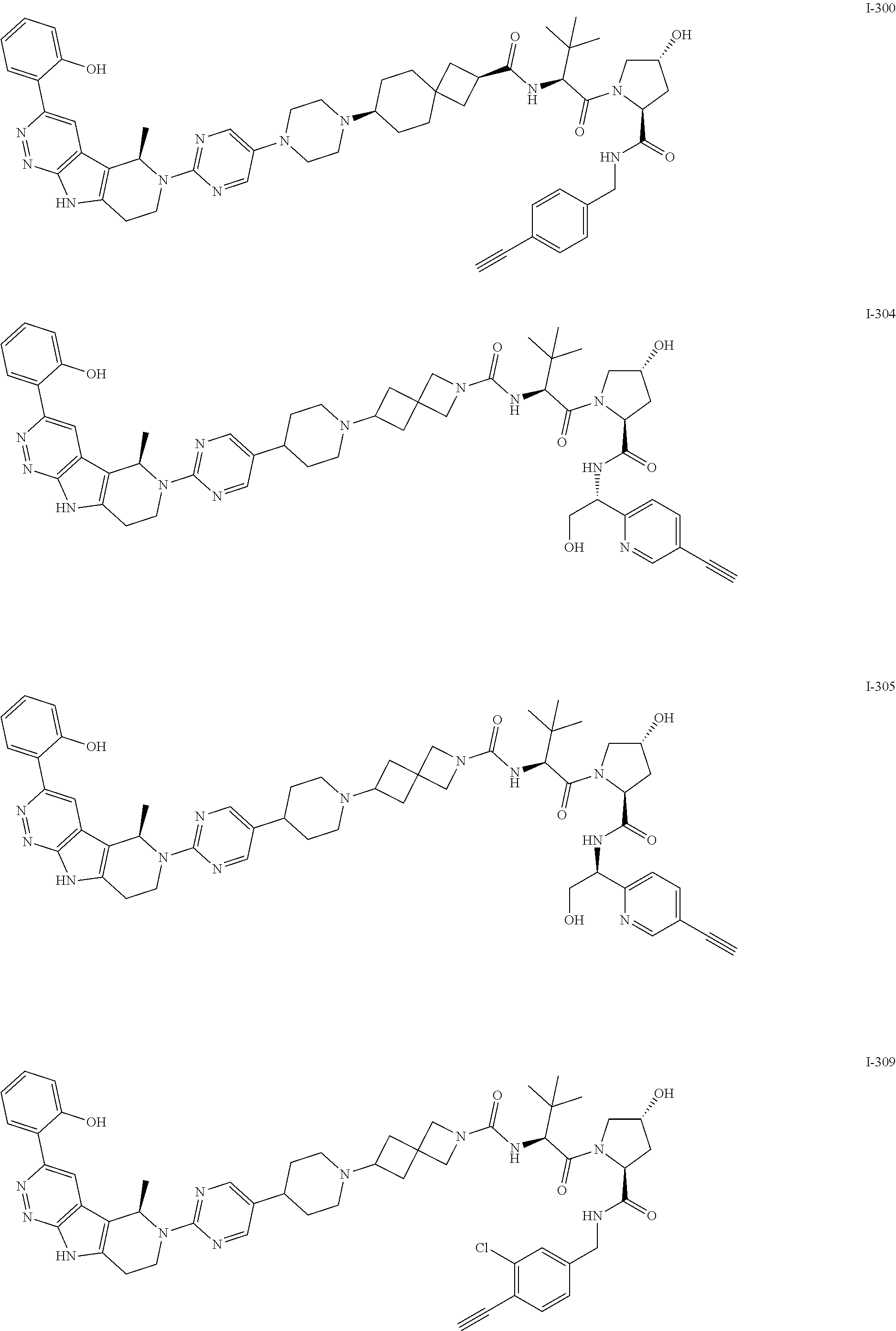 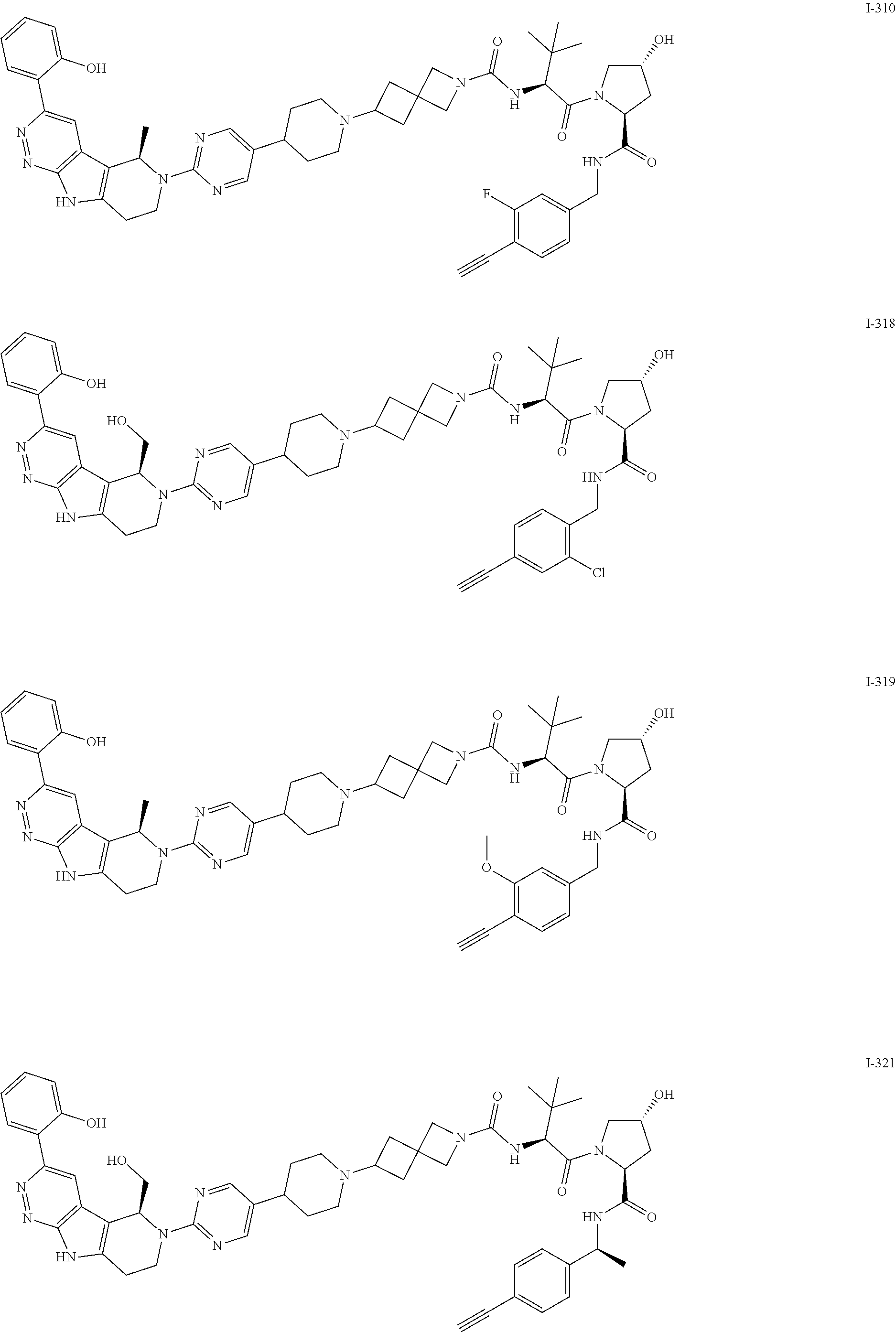 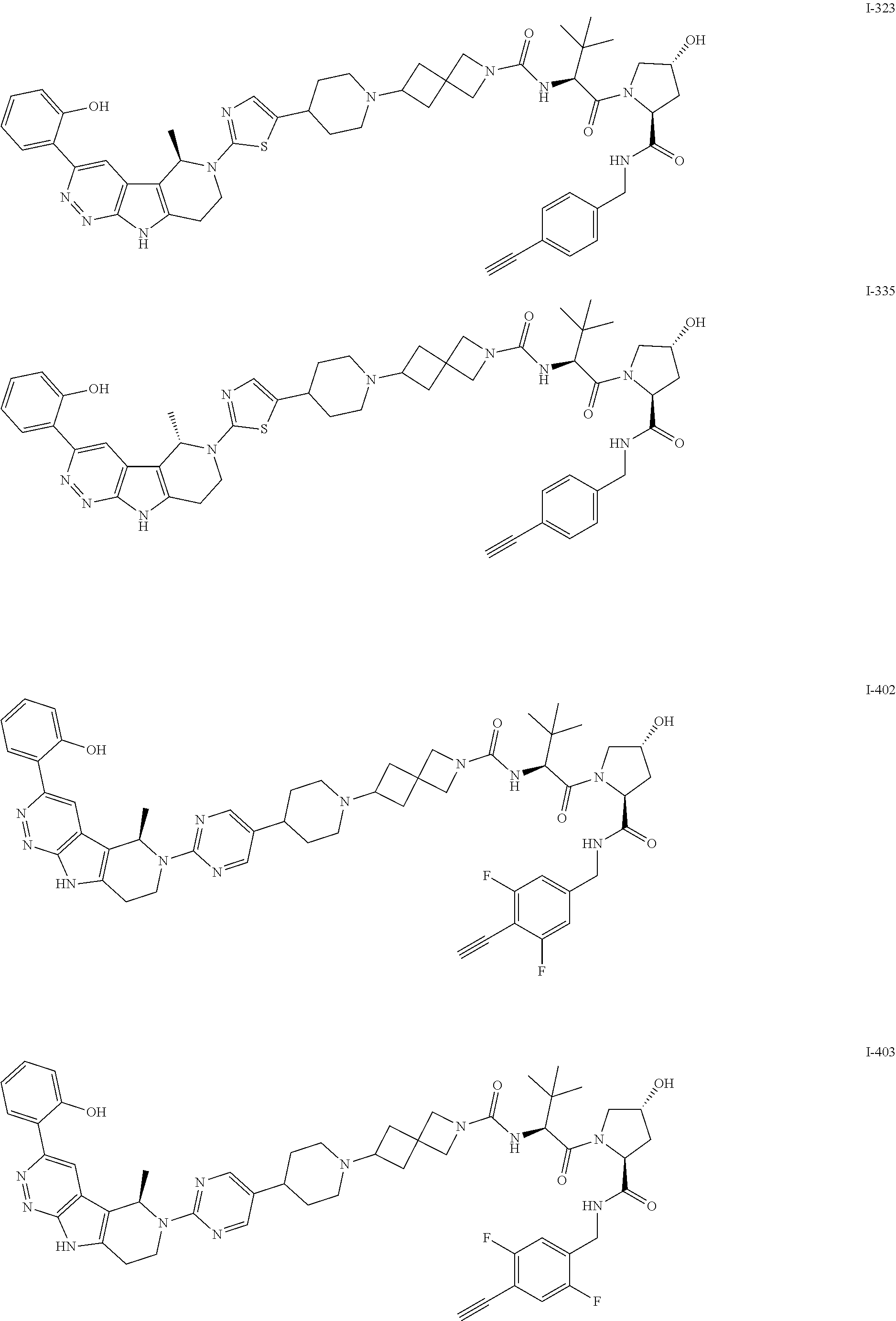 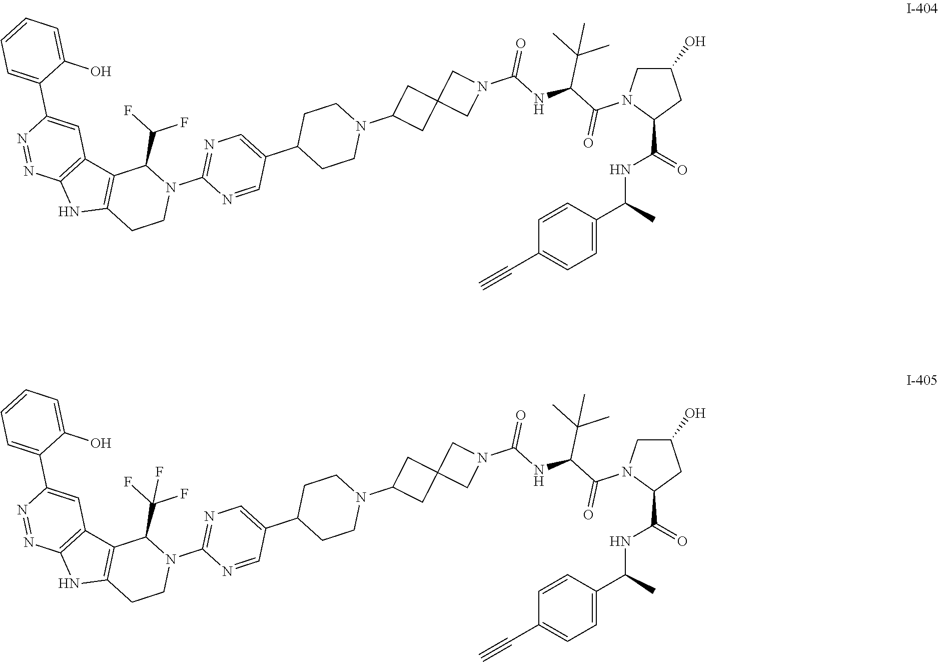 [SMILES: C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C(F)F)nc5)CC4)C3)C2)C(C)(C)C)cc1 ; C#Cc1ccc([C@H](C)NC(=O)[C@@H]2C[C@@H](O)CN2C(=O)[C@@H](NC(=O)N2CC3(CC(N4CCC(c5cnc(N6CCc7[nH]c8nnc(-c9ccccc9O)cc8c7[C@H]6C(F)(F)F)nc5)CC4)C3)C2)C(C)(C)C)cc1] 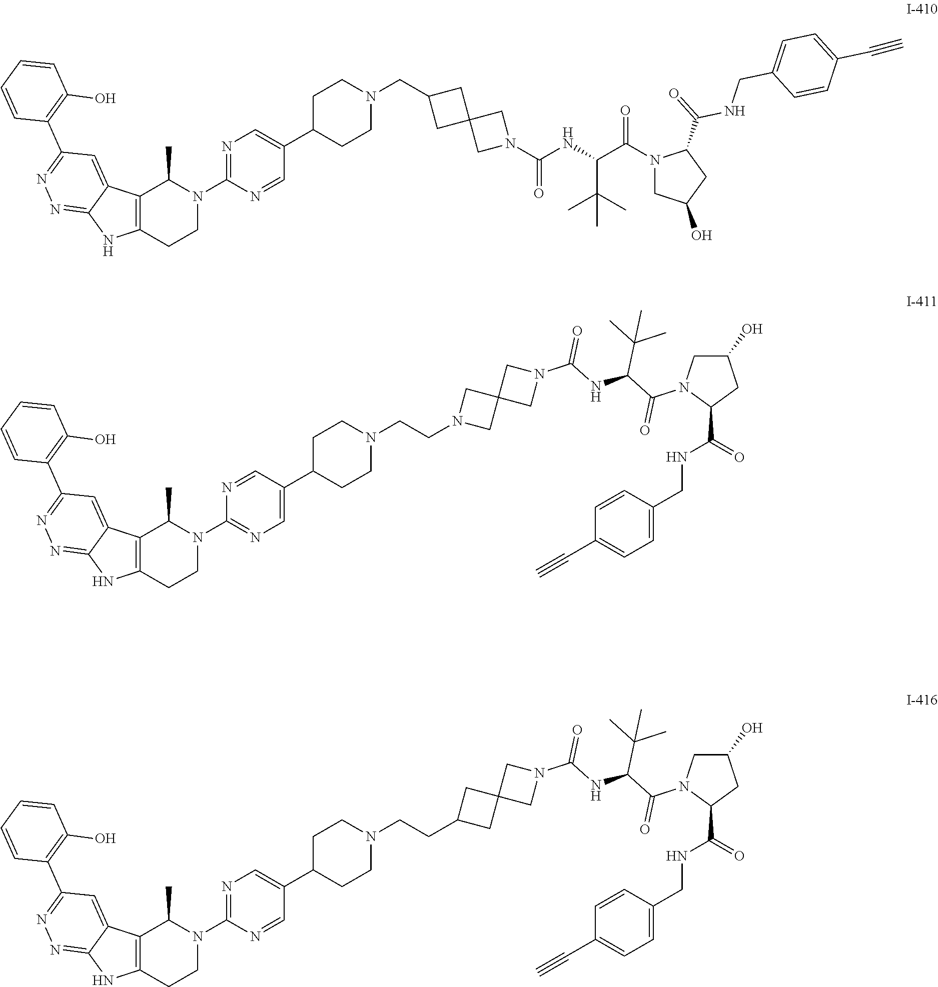 [SMILES: C#Cc1ccc(CNC(=O)[C@@H]2C[C@@H](O)CN2C(=O)[C@@H](NC(=O)N2CC3(CC(CN4CCC(c5cnc(N6CCc7[nH]c8nnc(-c9ccccc9O)cc8c7[C@H]6C)nc5)CC4)C3)C2)C(C)(C)C)cc1 ; C#Cc1ccc(CNC(=O)[C@@H]2C[C@@H](O)CN2C(=O)[C@@H](NC(=O)N2CC3(CN(CCN4CCC(c5cnc(N6CCc7[nH]c8nnc(-c9ccccc9O)cc8c7[C@H]6C)nc5)CC4)C3)C2)C(C)(C)C)cc1 ; C#Cc1ccc(CNC(=O)[C@@H]2C[C@@H](O)CN2C(=O)[C@@H](NC(=O)N2CC3(CC(CCN4CCC(c5cnc(N6CCc7[nH]c8nnc(-c9ccccc9O)cc8c7[C@H]6C)nc5)CC4)C3)C2)C(C)(C)C)cc1]
or a pharmaceutically acceptable salt thereof.

16. A pharmaceutical composition comprising a compound of claim 1, and a pharmaceutically acceptable carrier, adjuvant, or vehicle.

17. A method of degrading one or more of SMARCA2, SMARCA4, and PB1 protein in a patient or biological sample comprising administering to said patient, or contacting said biological sample with a compound of claim 1, or a pharmaceutical composition thereof.

18. A method of treating one or more SMARCA2-mediated, SMARCA4-mediated, or PB1-mediated disorder, disease, or condition in a patient comprising administering to said patient a compound of claim 1, or a pharmaceutical composition thereof.

19. The method of claim 18, wherein the one or more SMARCA2-mediated, SMARCA4-mediated, or PB1-mediated disorder, disease or condition is selected from a cancer, a neurodegenerative disease, a viral disease, an autoimmune disease, an inflammatory disorder, a hereditary disorder, a hormone-related disease, a metabolic disorder, a condition associated with organ transplantation, an immunodeficiency disorder, a destructive bone disorder, a proliferative disorder, an infectious disease, a condition associated with cell death, thrombin-induced platelet aggregation, liver disease, a pathologic immune condition involving T cell activation, a cardiovascular disorder, and a CNS disorder.

20. The method of claim 19, wherein the cancer is selected from lung cancer, non-small cell lung cancer (NSCLC), small-cell lung cancer, glioma, breast cancer, pancreatic cancer, colorectal cancer, bladder cancer, endometrial cancer, penile cancer, esophagogastric cancer, hepatobiliary cancer soft tissue sarcoma, ovarian cancer, head and neck cancer, renal cell carsinoma, bone cancer, non-Hodgkin lymphoma, prostate cancer, embryonal tumors, germ cell tumors, cervical cancer, thyroid cancer, salivary gland cancer, gastrointestinal neuroendocrine tumor, uterine sarcoma, gastrointestinal stromal tumor, CNS cancer, thymic tumor, adrenocortical carcinoma, appendiceal cancer, small bowel cancer, non-melanoma skin cancer, melanoma, leukemia, and malignant rhabdoid tumors (MRT).
